(19) United States
(12) Patent Application Publication
(10) Pub. No.: US 2026/0184691 A1
(43) Pub. Date: Jul. 2, 2026

(54) INHIBITORS AND DEGRADERS OF PIP4K PROTEIN

(71) Applicant: Larkspur Biosciences, Inc., Cambridge, MA (US)

(72) Inventors: Krista B. GOODMAN, Belmont, MA (US); Guosen YE, Waltham, MA (US); Morgan O'SHEA, Waltham, MA (US)

(21) Appl. No.: 19/129,925

(22) PCT Filed: Nov. 14, 2023
(86) PCT No.: PCT/US2023/079669

§ 371 Date: May 14, 2025

Related U.S. Application Data
(60) Provisional application No. 63/425,648, filed on Nov. 15, 2022.
(60) Provisional application No. 63/427,029, filed on Nov. 21, 2022.
(60) Provisional application No. 63/591,331, filed on Oct. 18, 2023.

(51) Int. Cl. C07D 401/14; A61K 31/506; A61K 31/5377; C07D 405/14; C07D 413/14; C07D 471/10; C07D 487/10

(52) U.S. Cl. CPC C07D 401/14; A61K 31/506; A61K 31/5377; C07D 405/14; C07D 413/14; C07D 471/10; C07D 487/10

(57) ABSTRACT

Provided are compounds and compositions which modulate the level or activity of PIP4K2C. Also provided are methods for treating diseases or conditions by modulating (e.g., reducing) the level or activity of a PIP4K2C, comprising administering the compounds and compositions.

### CROSS-REFERENCE TO RELATED APPLICATIONS

This application claims priority to U.S. Provisional Application No. 63/425,648, filed Nov. 15, 2022; U.S. Provisional Application No. 63/427,029, filed Nov. 21, 2022; and U.S. Provisional Application No. 63/591,331, filed Oct. 18, 2023; the contents of which are hereby incorporated by reference in their entirety for all purposes.

### BACKGROUND

The phosphatidylinositol 5-phosphate 4-kinases (PI5P4Ks), consisting of the three isoforms, PISP4Kα, β, and γ, are lipid kinases that catalyze phosphorylation of phosphatidylinositol 5-phosphate (PI5P) on its 4-position to form phosphatidylinositol-4,5-bisphosphate (PI-4,5-P2) (Rameh et al., Nature 390(6656):192-196 (1997). In the cellular membrane, PI-4,5-P2 is also produced by another signaling pathway in which phosphatidylinositol 4-phosphate (PI4P) is phosphorylated by phosphatidylinositol 4-phosphate 5-kinases (PI4P5Ks).

Although the majority of PI-4,5-P2 is produced via the PI4P5K pathway, PI5P4Ks have been recognized as key regulators of many cell functions including metabolism, stress response, autophagy, and immunological processes (Hu et al., J. Lipid Res. 59:507-514 (2018); Lamia et al., Mol. Cell. Biol. 24(11):5080-5087 (2004); Shim et al., Proc. Natl. Acad. Sci. U.S.A 113(27):7596-7601 (2016); Al-Ramahi et al., eLife 6:e29123 (2017); Lundquist et al., Mol. Cell 70(3):531-543 (2018); Bulley et al., Proc. Natl. Acad. Sci. U.S.A 113(38):10571-10576 (2016); Keune et al., Adv. Biol. Regul. 53(2):179-189 (2013)). PIP4Ks have distinct catalytic and non-catalytic functions in controlling cellular metabolism and suppress PIP5K activity and insulin-stimulated production of PI(3,4,5)P3 (Wang et al., Cell Rep. 27:1991-2001 (2019)).

Dysregulation of the PI5P4K signaling pathway has been further linked to diseases such as diabetes, neurodegenerative disorders, and cancers (Lamia et al., Mol. Cell. Biol. 24(11): 5080-5087 (2004); Al-Ramahi et al., eLife 6:e29123 (2017); Jude et al., Oncogene 34(10):1253-1262 (2015); Luoh et al., Oncogene 23:1354-1363 (2004); Emerling et al., Cell 155(4):844-857 (2013). Analysis of PI(4,5)P2 levels in cells with single or double knockdown of PIP4K isoforms revealed an additive effect among all three isoforms that does not correlate with their relative catalytic activities. Double knockdown of the most active isoforms, PIP4KA/B, failed to phenocopy the triple knockdown, suggesting that catalytic activity is not the most important factor in regulating PI(4,5)P2 levels (Wang et al, Cell Rep. 27:1991-2001 (2019), and suggesting a unique role for PIP4KC.

These findings indicate that the inhibition of PI5P4K kinase activity and/or degradation of PI4P4K might have therapeutic potential across various diseases.

### SUMMARY OF THE INVENTION

In one aspect, provided is a compound of Formula (I′):

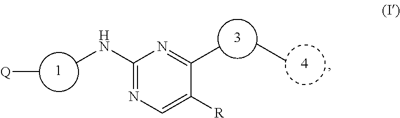

or a tautomer or stereoisomer thereof, or a pharmaceutically acceptable salt of any of the foregoing, wherein Q, R, Ring 1, Ring 3, and Ring 4 are as detailed herein.

In one aspect, provided is a compound of Formula (I):

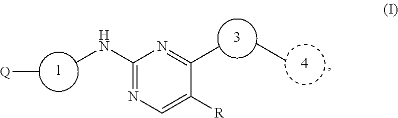

or a tautomer or stereoisomer thereof, or a pharmaceutically acceptable salt of any of the foregoing, wherein Q, R, Ring 1, Ring 3, and Ring 4 are as detailed herein. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Q is Ring 5, which is as detailed herein. In some embodiments, provided is a compound of Formula (II):

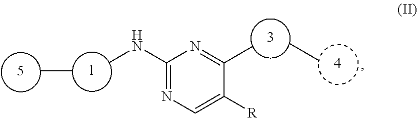

or a tautomer or stereoisomer thereof, or a pharmaceutically acceptable salt of any of the foregoing, wherein R, Ring 1, Ring 3, Ring 4, and Ring 5 are as detailed herein. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Q is of formula (i)

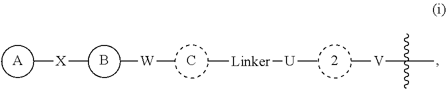

wherein Ring A, X, Ring B, W, Ring C, Linker, U, Ring 2, and V are as detailed herein. In some embodiments, provided is a compound of Formula (III):

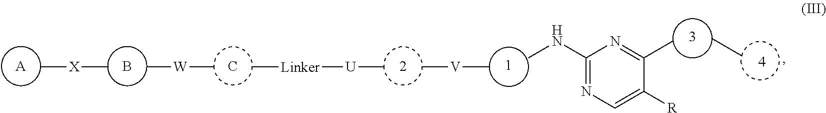

or a tautomer or stereoisomer thereof, or a pharmaceutically acceptable salt of any of the foregoing, wherein Ring A, X, Ring B, W, Ring C, Linker, U, Ring 2, V, Ring 1, Ring 3, and Ring 4 are as detailed herein. In some variations, the embodiments provided herein also apply to a compound of Formula (I′), or a stereoisomer or tautomer thereof, or a pharmaceutically acceptable salt of any of the foregoing, or any variation or embodiment thereof.

In another aspect, provided is a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In some embodiments, provided is a pharmaceutical composition comprising a therapeutically effective amount of a compound of Formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In another variation, provided is a pharmaceutical composition comprising a compound of Formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In another aspect, provided is a pharmaceutical composition comprising a compound of Formula (I′), or any related formula such as (T), (I-a), (I-b), (I-c), (T-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier.

In another aspect, provided is a method of making a compound of formula (T), or any related formula such as (I-a), (I-b), (I-c), (T-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing. In another variation, provided is a method of making a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing.

In another aspect, provided is a method of treating a disease or disorder comprising modulating (e.g., reducing) the activity or level of at least one of PIP4K2A, PIP4K2B, and PIP4K2C, comprising administering (a) a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In some embodiments, provided is a method of treating a disease or disorder comprising modulating (e.g., reducing) the activity or level of at least one of PIP4K2A, PIP4K2B, and PIP4K2C, comprising administering (a) a therapeutically effective amount of the compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In another variation, provided is a method of treating a disease or disorder comprising modulating (e.g., reducing) the activity or level of at least one of PIP4K2A, PIP4K2B, and PIP4K2C, comprising administering (a) a compound of Formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In some embodiments, provided is a method of treating a disease or disorder comprising modulating (e.g., reducing) the activity or level of at least one of PIP4K2A, PIP4K2B, and PIP4K2C, comprising administering (a) a therapeutically effective amount of the compound of Formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier.

In another aspect, provided is a method of treating a disease or disorder comprising modulating (e.g., reducing) the activity of at least one of PIP4K2A, PIP4K2B, and PIP4K2C, comprising administering (a) a compound of formula (I), or any related formula such as (I-a), (I-b), (I-c), (I-d), (I-e), (I-f), (IT), (II-a), (II-b), (II-c), (II-d), (II-e), (II-f), (II-g), or (II-h), or a tautomer or stereoisomer thereof, or a pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some embodiments, provided is a method of treating a disease or disorder comprising modulating (e.g., reducing) the activity of at least one of PIP4K2A, PIP4K2B, and PIP4K2C, comprising administering (a) a therapeutically effective amount of the compound of formula (I), or any related formula such as (I-a), (I-b), (I-c), (I-d), (I-e), (I-f), (II), (II-a), (II-b), (II-c), (II-d), (II-e), (II-f), (II-g), or (II-h), or a tautomer or stereoisomer thereof, or a pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some embodiments, the method comprises reducing the activity of PIP4K2C. In another variation, provided is a method of treating a disease or disorder comprising modulating (e.g., reducing) the activity of at least one of PIP4K2A, PIP4K2B, and PIP4K2C, comprising administering (a) a compound of formula (I′), or any related formula such as (I), (I-a), (I-b), (I-c), (I-d), (I-e), (I-f), (II), (II-a), (II-b), (II-c), (II-d), (II-e), (II-f), (II-g), or (II-h), or a tautomer or stereoisomer thereof, or a pharmaceutically acceptable salt of any of the foregoing, or (b) a pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some embodiments, provided is a method of treating a disease or disorder comprising modulating (e.g., reducing) the activity of at least one of PIP4K2A, PIP4K2B, and PIP4K2C, comprising administering (a) a therapeutically effective amount of the compound of formula (I′), or any related formula such as (I), (I-a), (I-b), (I-c), (I-d), (I-e), (I-f), (II), (II-a), (II-b), (II-c), (II-d), (II-e), (II-f), (II-g), or (II-h), or a tautomer or stereoisomer thereof, or a pharmaceutically acceptable salt of any of the foregoing, or (b) a pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some embodiments, the method comprises reducing the activity of PIP4K2C.

In another aspect, provided is a method of treating a disease or disorder comprising modulating (e.g., reducing) the level of at least one of PIP4K2A, PIP4K2B, and PIP4K2C, comprising administering (a) a compound of formula (I), or any related formula such as (I-a), (I-b), (I-c), (I-d), (I-e), (I-f), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In another aspect, provided is a method of treating a disease or disorder comprising modulating (e.g., reducing) the level of at least one of PIP4K2A, PIP4K2B, and PIP4K2C, comprising administering (a) a therapeutically effective amount of the compound of formula (I), or any related formula such as (I-a), (I-b), (I-c), (I-d), (I-e), (I-f), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some embodiments, the method comprises reducing the level of PIP4K2C. In some embodiments, the method comprises degrading PIP4K2C. In some embodiments, the method comprises selectively reducing the level of PIP4K2C. In some embodiments, the method comprises selectively degrading PIP4K2C. In another aspect, provided is a method of treating a disease or disorder comprising modulating (e.g., reducing) the level of at least one of PIP4K2A, PIP4K2B, and PIP4K2C, comprising administering (a) a compound of formula (I′), or any related formula such as (I), (I-a), (I-b), (I-c), (I-d), (I-e), (I-f), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In another aspect, provided is a method of treating a disease or disorder comprising modulating (e.g., reducing) the level of at least one of PIP4K2A, PIP4K2B, and PIP4K2C, comprising administering (a) a therapeutically effective amount of the compound of formula (I′), or any related formula such as (I), (I-a), (I-b), (I-c), (I-d), (I-e), (I-f), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some embodiments, the method comprises reducing the level of PIP4K2C. In some embodiments, the method comprises degrading PIP4K2C. In some embodiments, the method comprises selectively reducing the level of PIP4K2C. In some embodiments, the method comprises selectively degrading PIP4K2C.

In another aspect, provided is a method of enhancing immune function in a subject in need thereof, comprising administering to a subject (a) a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In some embodiments, provided is a method of enhancing immune function in a subject in need thereof, comprising administering to a subject (a) a therapeutically effective amount of a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or pharmaceutically acceptable salt of any of the foregoing, or a (b) pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In another aspect, provided is a method of enhancing immune function in a subject in need thereof, comprising administering to a subject (a) a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or pharmaceutically acceptable salt of any of the foregoing, or (b) a pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In some embodiments, provided is a method of enhancing immune function in a subject in need thereof, comprising administering to a subject (a) a therapeutically effective amount of a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or pharmaceutically acceptable salt of any of the foregoing, or a (b) pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier.

In some aspects, the present invention is directed to methods of stimulating/activating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject (a) a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or pharmaceutically acceptable salt of any of the foregoing, or (b) a pharmaceutical composition comprising a compound of formula (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In some aspects, the present invention is directed to methods of stimulating/activating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject (a) a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or pharmaceutically acceptable salt of any of the foregoing, or (b) a pharmaceutical composition comprising a compound of formula (I′), or any related formula such as (I), (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier.

Any aspects or embodiments provided herein of a compound of formula (I), or a stereoisomer or tautomer thereof, or a pharmaceutically acceptable salt of any of the foregoing, or any variation or embodiment thereof, are also aspects or embodiments of a compound of formula (I′), or a stereoisomer or tautomer thereof, or a pharmaceutically acceptable salt of any of the foregoing, or any variation or embodiment thereof. Any embodiments provided herein of a compound of formula (I) or (I′), or a stereoisomer or tautomer thereof, or a pharmaceutically acceptable salt of any of the foregoing, or any variation or embodiment thereof, apply where applicable to any other formula detailed herein, the same as if each and every embodiment were specifically and individually listed. Thus, it is understood and described that each embodiment provided herein of a compound of formula (I) or (I′), or a stereoisomer or tautomer thereof, or a pharmaceutically acceptable salt of any of the foregoing, or any variation or embodiment thereof, such as embodiments related to Ring 1, Ring 2, Ring 3, Ring 4, Ring 5, Ring A, Ring B, Ring C, R, R¹, R^{2a}, R^{2b}, R^{3a}, R^{3b}, R^{4a}, R^{4b}, R⁵, R^{C}, R^{D}, R^{L}, R^{V}, R^{w}, R^{x}, R^{y}, R^{z}, Q, U, V, X, W, Linker, L¹, L², L³, and n apply to any one of formulae (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e) the same as if each and every embodiment were specifically and individually listed. It is also understood and described that all such embodiments may be used in any of the pharmaceutical compositions, methods, kits, uses, etc. comprising such compounds or other aspects detailed herein.

As shown in working examples, compounds embraced by formula (I′) and formula (I) inhibit the activity or promote the degradation of at least one of PIP4K2A, PIP4K2B, and PIP4K2C.

Without intending to be bound by any particular theory, the compounds of formula (III) and any related formula such as, e.g., (III-a), (III-b), (III-c), (III-d), and (III-e), are believed to cause degradation of at least one of PIP4K2A, PIP4K2B, and PIP4K2C by recruitment of cells' Ubiquitin/Proteasome System, whose function is to routinely identify and remove damaged proteins, into close proximity with PIP4K2A, PIP4K2B, or PIP4K2C as a result of binding between PIP4K2A, PIP4K2B, or PIP4K2C, and the targeting ligand. After destruction of a PIP4K2A, PIP4K2B, or PIP4K2C protein, the degrader is released and continues to be active. Thus, by engaging and exploiting the body's own natural protein disposal system, the compounds of the present invention may represent a potential improvement over current small molecule inhibitors of PIP4K2A, PIP4K2B, and PIP4K2C and may overcome one or more limitations regarding their use. Therefore, effective intracellular concentrations of the degraders may be significantly lower than for small molecule PIP4K2A, PIP4K2B, or PIP4K2C inhibitors. Also, although PIP4K2A, PIP4K2B, and/or PIP4K2C genetic knockdown or knockout can be used to reduce the cellular concentration or amount of these proteins, it may be preferable to post-translationally disrupt, degrade, or destabilize PIP4K2A, PIP4K2B, and/or PIP4K2C proteins. Targeting proteins directly, rather than via the DNA or mRNA molecules that encode them, is a more direct and rapid method for reducing the scaffolding function of PIP4K proteins. Hence, degradation can allow some PIP4K2A, PIP4K2B, and/or PIP4K2C catalytic function to proceed while reducing the non-catalytic functions such as scaffolding between PIP4K2A, PIP4K2B, and/or PIP4K2C proteins and other cellular proteins and structures. Collectively, the present compounds may represent a set of new chemical tools for at least one of PIP4K2A, PIP4K2B, and PIP4K2C knockdown and may provide a potential treatment modality for PIP4K2A, PIP4K2B, and/or PIP4K2C-associated cancers and autophagy-dependent diseases (e.g., neurodegenerative disorders, insulin). The compounds may be used to enhance immune functions as described in, e.g., WO 2020/210686 and Wang et al., Cell Rep. 27:1991-2001 (2019), each of which is incorporated herein by reference in its entirety. Inhibitors and degraders of PIP4K protein and methods for treating diseases or conditions by modulating the level or activity of at least one of PIP4K2A, PIP4K2B, and PIP4K2C are also disclosed in WO 2022/246025, herein incorporated by reference in its entirety.

### DETAILED DESCRIPTION

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, “about X” includes and describes “X” per se.

“Alkyl” as used herein refers to and includes, unless otherwise stated, a saturated linear (i.e., unbranched) or branched univalent hydrocarbon chain or combination thereof, having the number of carbon atoms designated (i.e., C₁₋₁₀ means one to ten carbon atoms). Particular alkyl groups are those having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Particular C₁-C₄ alkyl groups include C₁-C₃ alkyl groups. Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkylene” as used herein refers to the same residues as alkyl, but having bivalency. Particular alkylene groups are those having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Cycloalkyl” as used herein refers to and includes, unless otherwise stated, saturated cyclic univalent hydrocarbon structures, having the number of carbon atoms designated (i.e., C₃-C₁₀ means three to ten carbon atoms). Cycloalkyl can consist of one ring (e.g., cyclohexyl), or multiple rings (e.g., adamantyl). In polycyclic ring systems, one or more of the fused rings can be cycloalkyl or aryl (e.g., spiro[4.5]decanyl, or 1,2,3,4-tetrahydronaphthalenyl), but excludes heterocyclyl or heteroaryl groups. A cycloalkyl comprising more than one ring may be fused, spiro or bridged, or combinations thereof. Particular cycloalkyl groups are those having from 3 to 12 annular carbon atoms (a “C₃₋₁₂ cycloalkyl”), having 3 to 10 annular carbon atoms (a “C₃₋₁₀ cycloalkyl”), having 3 to 8 annular carbon atoms (a “C₃-C₈ cycloalkyl”), having 3 to 6 annular carbon atoms (a “C₃-C₆ cycloalkyl”), or having from 3 to 4 annular carbon atoms (a “C₃-C₄ cycloalkyl”). Examples of cycloalkyl include, but are not limited to, cyclopropyl, cyclobutyl, cyclopentyl, cyclohexyl, cycloheptyl, norbornyl, and the like. A cycloalkyl group having more than one ring where at least one ring is aromatic may be connected to the parent structure at either an aromatic ring position or at a non-aromatic ring position. In one variation, a cycloalkyl group having more than one ring where at least one ring is aromatic is connected to the parent structure at an aromatic ring position. In another variation, a cycloalkyl group having more than one ring where at least one ring is aromatic is connected to the parent structure at a non-aromatic ring position.

“Aryl” or “Ar” as used herein refers to an unsaturated aromatic carbocyclic group having a single ring (e.g., phenyl) or multiple condensed rings (e.g., naphthyl or anthryl) which condensed rings must be aromatic. Particular aryl groups are those having from 6 to 14 annular carbon atoms (a “C₆-C₁₄ aryl”).

“Heteroaryl” as used herein refers to an unsaturated aromatic cyclic group having from 1 to 14 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings must be aromatic. Particular heteroaryl groups are 5 to 14-membered rings having 1 to 12 annular carbon atoms and 1 to 6 annular heteroatoms independently selected from nitrogen, oxygen and sulfur, 5 to 10-membered rings having 1 to 8 annular carbon atoms and 1 to 4 annular heteroatoms independently selected from nitrogen, oxygen and sulfur, or 5, 6 or 7-membered rings having 1 to 5 annular carbon atoms and 1 to 4 annular heteroatoms independently selected from nitrogen, oxygen and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen and sulfur. A heteroaryl group may be connected to the parent structure at a ring carbon atom or a ring heteroatom.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof. In fused ring systems, one or more of the fused rings can be cycloalkyl, aryl, or heteroaryl groups. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur. A heterocyclyl group having more than one ring where at least one ring is aromatic may be connected to the parent structure at either an aromatic ring position or at a non-aromatic ring position. In one variation, a heterocyclyl group having more than one ring where at least one ring is aromatic is connected to the parent structure at an aromatic ring position. In another variation, a heterocyclyl group having more than one ring where at least one ring is aromatic is connected to the parent structure at a non-aromatic ring position.

“Halo” or “halogen” refers to elements of the Group 7 of the periodic table. Preferred halo groups include fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Oxo” refers to the moiety ═O.

“Optionally substituted” unless otherwise specified means that a group may be unsubstituted or substituted with one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography (HPLC), and chiral supercritical fluid chromatography (SFC). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof (e.g., racemic mixtures), and includes “enantiomers,” which refers to two stereoisomers whose structures are non-superimposable mirror images of one another. “Diastereomers” are stereoisomers that have at least two asymmetric atoms, but which are not mirror images of each other.

### Compounds of Formula (I′) or (I)

In one aspect, provided is a compound of Formula (I′):

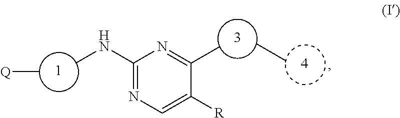

or a tautomer or stereoisomer thereof, or a pharmaceutically acceptable salt of any of the foregoing, wherein:
R is halo;
Ring 1 is pyridyl optionally substituted with one or more R¹;
Ring 3 is a ring selected from the group consisting of 3- to 10-membered cycloalkyl optionally substituted with one or more R^{3a}, 4- to 10-membered heterocyclyl optionally substituted with one or more R^{3a}, 6- to 10-membered aryl optionally substituted with one or more R^{3b}, or 5- to 10-membered heteroaryl optionally substituted with one or more R^{3b};
Ring 4 is optional, and when present is a ring selected from the group consisting of 3- to 10-membered cycloalkyl optionally substituted with one or more R^{4a}, 4- to 10-membered heterocyclyl optionally substituted with one or more R^{4a}, 6- to 10-membered aryl optionally substituted with one or more R^{4b}, and 5- to 10-membered heteroaryl optionally substituted with one or more R^{4b};
Q is Ring 5, wherein Ring 5 is a pyrrolidinone or imidazolidinone, each of which is optionally substituted with one or more R⁵, or
Q is of formula (i)

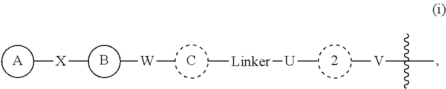

wherein:
Ring A is selected from the group consisting of

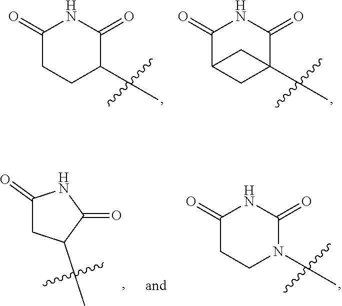 [SMILES: *C1CCC(=O)NC1=O ; *C12CC(C1)C(=O)NC2=O ; *C1CC(=O)NC1=O ; *N1CCC(=O)NC1=O]

and, each of which is optionally substituted with C₁₋₄alkyl or halo;
X is a bond, —O—, —NH—, or —NHC(O)—;
Ring B is selected from the group consisting of

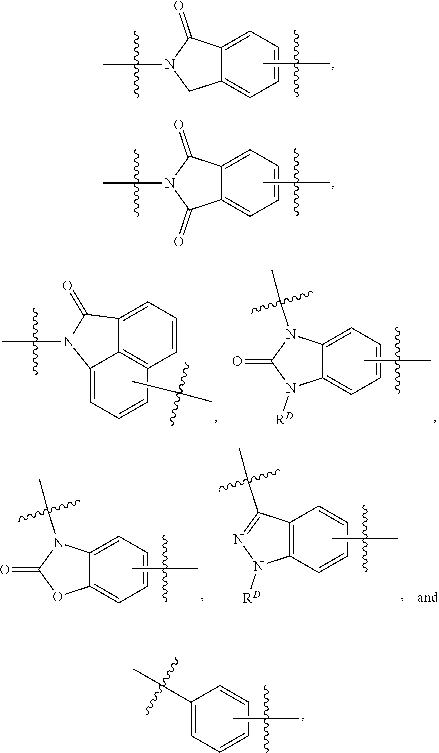

each of which is optionally substituted with one or more halo, C₁₋₄alkyl, C₁₋₄haloalkyl, or OH, wherein R^{D} is H or C₁₋₄alkyl;
W is a bond, —O—, —NH—, or —NHC(O)—;
Ring C is optional, and when present is 4- to 12-membered heterocyclyl optionally substituted with one or more R^{C};
Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
   * indicates attachment to Ring C when Ring C is present, and * indicates attachment to W when Ring C is absent;
   ** indicates attachment to U when Ring 2 is present, and ** indicates attachment to V when Ring 2 is absent;
   L¹ is a bond or C₁₋₆ alkylene;
   L² is a 3- to 10-membered cycloalkyl or 4- to 12-membered heterocyclyl optionally substituted with one or more R^{L}; and
   L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
U is absent when Ring 2 is absent, and is a bond or C(O) when Ring 2 is present;
Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
V is a bond, —NR^{V}—, —C(O)—, —C(O)NH—, or —NHC(O)—;
each R¹ is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-(3- to 6-membered cycloalkyl), C₁₋₄alkylene-N(R^{y})—C₁₋₄alkyl, C₁₋₄alkylene-S(O)ₙR^{x}, or oxo;
each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, O-phenyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R⁵ is independently C₁₋₄alkyl, or two R⁵ are taken together with the atom(s) to which they are attached to form a four- to six-membered ring which is optionally substituted with C₁₋₄alkyl or C(O)C₁₋₄alkyl;
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{V} is independently halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with oxo;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
   provided that, when Ring 3 and Ring 4 are each unsubstituted phenyl and Q is Ring 5, Ring 5 is substituted with one or more R⁵.

In one aspect, provided is a compound of Formula (I):

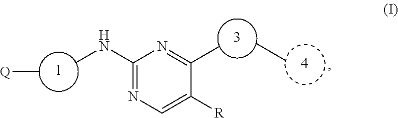

or a tautomer or stereoisomer thereof, or a pharmaceutically acceptable salt of any of the foregoing, wherein:
R is halo;
Ring 1 is pyridyl optionally substituted with one or more R¹;
Ring 3 is a monocyclic ring selected from the group consisting of 3- to 6-membered cycloalkyl optionally substituted with one or more R^{3a}, 4- to 6-membered heterocyclyl optionally substituted with one or more R^{3a}, phenyl optionally substituted with one or more R^{3b}, or 5- to 6-membered heteroaryl optionally substituted with one or more R^{3b};
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Q is Ring 5, wherein Ring 5 is a pyrrolidinone or imidazolidinone, each of which is optionally substituted with one or more R⁵, or
Q is of formula (i)

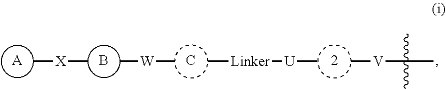

wherein:
Ring A is selected from the group consisting of

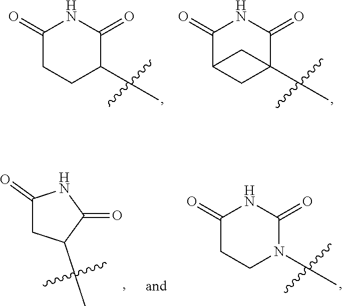 [SMILES: *C1CCC(=O)NC1=O ; *C12CC(C1)C(=O)NC2=O ; *C1CC(=O)NC1=O ; *N1CCC(=O)NC1=O]

and, each of which is optionally substituted with C₁₋₄alkyl or halo;
X is a bond, —O—, —NH—, or —NHC(O)—;
Ring B is selected from the group consisting of

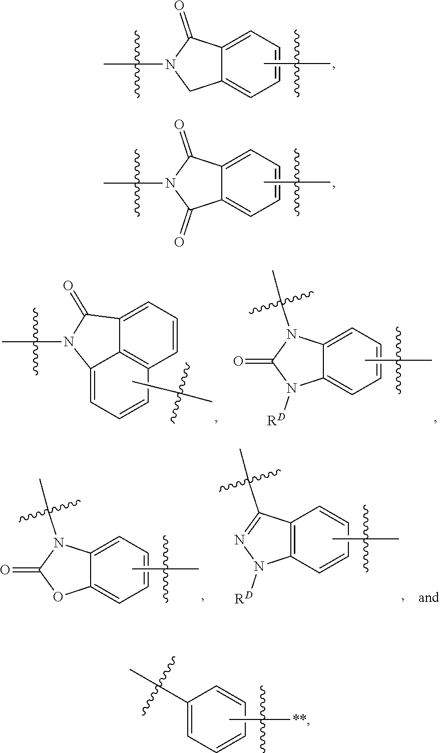

each of which is optionally substituted with one or more halo, C₁₋₄alkyl, C₁₋₄haloalkyl, or OH, wherein R^{D} is H or C₁₋₄alkyl;
W is a bond, —O—, —NH—, or —NHC(O)—;
Ring C is optional, and when present is 4- to 12-membered heterocyclyl optionally substituted with one or more R^{C};
Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
   * indicates attachment to Ring C when Ring C is present, and * indicates attachment to W when Ring C is absent;
   ** indicates attachment to U when Ring 2 is present, and ** indicates attachment to V when Ring 2 is absent;
   L¹ is a bond or C₁₋₆ alkylene;
   L² is a 3- to 10-membered cycloalkyl or 4- to 12-membered heterocyclyl optionally substituted with one or more R^{L}; and
   L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
U is absent when Ring 2 is absent, and is a bond or C(O) when Ring 2 is present;
Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
V is a bond, —NR^{V}—, —C(O)—, —C(O)NH—, or —NHC(O)—;
each R¹ is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₐN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R⁵ is independently C₁₋₄alkyl, or two R⁵ are taken together with the atom(s) to which they are attached to form a four- to six-membered ring which is optionally substituted with C₁₋₄alkyl or C(O)C₁₋₄alkyl;
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{V} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
provided that, when Ring 3 and Ring 4 are each unsubstituted phenyl and Q is Ring 5, Ring 5 is substituted with one or more R⁵.
   In some variations, the embodiments provided herein also apply to a compound of formula (I′), or a stereoisomer or tautomer thereof, or a pharmaceutically acceptable salt of any of the foregoing, or any variation or embodiment thereof.

In one aspect, provided is a compound of Formula (I):

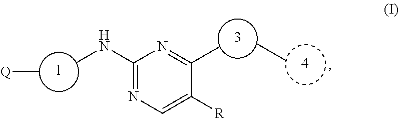

or a tautomer or stereoisomer thereof, or a pharmaceutically acceptable salt of any of the foregoing, wherein:
R is halo;
Ring 1 is pyridyl optionally substituted with one or more R¹;
Ring 3 is a monocyclic ring selected from the group consisting of 3- to 6-membered cycloalkyl optionally substituted with one or more R^{3a}, 4- to 6-membered heterocyclyl optionally substituted with one or more R^{3a}, phenyl optionally substituted with one or more R^{3b}, or 5- to 6-membered heteroaryl optionally substituted with one or more R^{3b};
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Q is Ring 5, wherein Ring 5 is a pyrrolidinone or imidazolidinone, each of which is optionally substituted with one or more R⁵, or
Q is of formula (i)

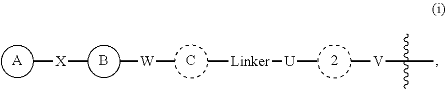

wherein:
Ring A is selected from the group consisting of

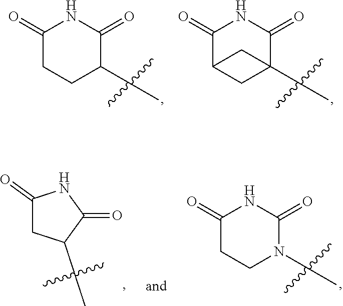 [SMILES: *C1CCC(=O)NC1=O ; *C12CC(C1)C(=O)NC2=O ; *C1CC(=O)NC1=O ; *N1CCC(=O)NC1=O]

each of which is optionally substituted with C₁₋₄alkyl or halo;
X is a bond, —O—, —NH—, or —NHC(O)—;
Ring B is selected from the group consisting of

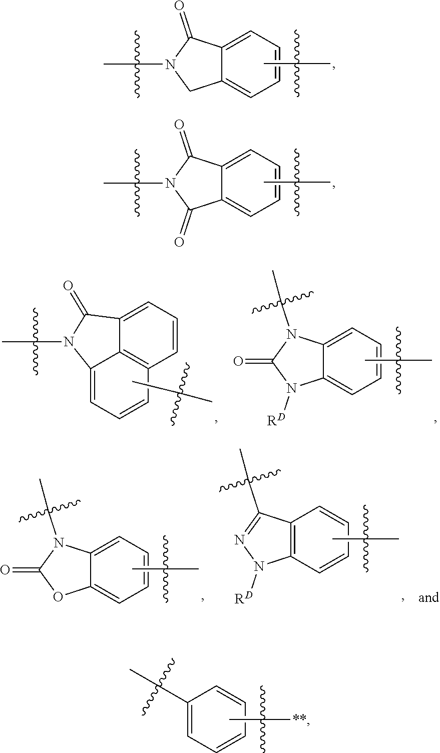

each of which is optionally substituted with one or more halo or OH, wherein R^{D} is H or C₁₋₄alkyl;
W is a bond, —O—, —NH—, or —NHC(O)—;
Ring C is optional, and when present is 4- to 12-membered heterocyclyl optionally substituted with one or more R^{C};
Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
   * indicates attachment to Ring C when Ring C is present, and * indicates attachment to W when Ring C is absent;
   ** indicates attachment to U when Ring 2 is present, and ** indicates attachment to V when Ring 2 is absent;
   L¹ is a bond or C₁₋₆ alkylene;
   L² is a 3- to 10-membered cycloalkyl or 4- to 12-membered heterocyclyl optionally substituted with one or more R^{L}; and
   L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
U is absent when Ring 2 is absent, and is a bond or C(O) when Ring 2 is present;
Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
V is a bond, —C(O)—, —C(O)NH—, or —NHC(O)—;
each R¹ is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R⁵ is independently C₁₋₄alkyl, or two R⁵ are taken together with the atom(s) to which they are attached to form a four- to six-membered ring which is optionally substituted with C₁₋₄alkyl or C(O)C₁₋₄alkyl;
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
   provided that, when Ring 3 and Ring 4 are each unsubstituted phenyl and Q is Ring 5, Ring 5 is substituted with one or more R⁵.
   In some variations, the embodiments provided herein also apply to a compound of formula (I′), or a stereoisomer or tautomer thereof, or a pharmaceutically acceptable salt of any of the foregoing, or any variation or embodiment thereof.

In some embodiments of the compound of Formula (I), R is chloro, fluoro, or bromo. In some embodiments of the compound of Formula (I), R is chloro or fluoro. In some embodiments of the compound of Formula (I), R is chloro. In some embodiments of the compound of Formula (I), R is fluoro. In some variations, the embodiments provided herein also apply to a compound of formula (I′), or a stereoisomer or tautomer thereof, or a pharmaceutically acceptable salt of any of the foregoing, or any variation or embodiment thereof.

In some embodiments of the compound of Formula (I), Ring 1 is

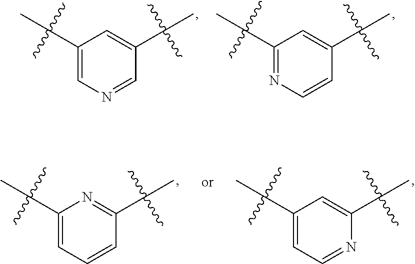 [SMILES: *c1cncc(*)c1 ; *c1ccnc(*)c1 ; *c1cccc(*)n1 ; *c1ccnc(*)c1]

each of which is optionally substituted with one or more R¹, wherein each R¹ is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl. In some embodiments, Ring 1 is

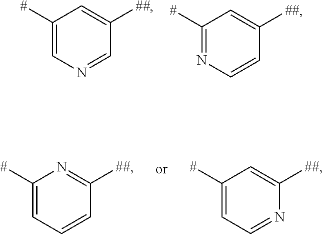

each of which is optionally substituted with one or more R¹, wherein # indicates attachment to Q and ## indicates attachment to the aminopyrimidine of the compound of Formula (I). In some embodiments, Ring 1 is

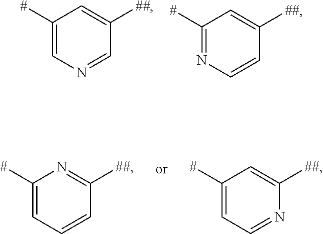

wherein # indicates attachment to Q and ## indicates attachment to the aminopyrimidine of the compound of Formula (I). In some embodiments, Ring 1 is

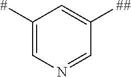

optionally substituted with one or more R¹. In some embodiments, Ring 1 is

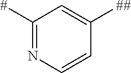

optionally substituted with one or more R¹. In some embodiments, Ring 1 is

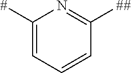

optionally substituted with one or more R¹. In some embodiments, Ring 1 is

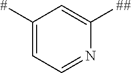

optionally substituted with one or more R¹. In some embodiments, Ring 1 is

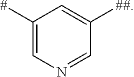

In some embodiments, Ring 1 is

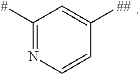

In some embodiments, Ring 1 is

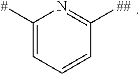

In some embodiments, Ring 1 is

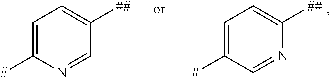

In some embodiments, Ring 1 is

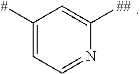

each of which is optionally substituted with one or more R¹, wherein # indicates attachment to Q and ## indicates attachment to the aminopyrimidine of the compound of Formula (I). In some embodiments, Ring 1 is

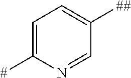

optionally substituted with one or more R¹. In some embodiments, Ring 1 is

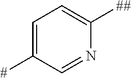

optionally substituted with one or more R¹. In some embodiments, Ring 1 is

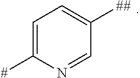

In some embodiments, Ring 1 is

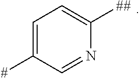

In some variations, the embodiments provided herein also apply to a compound of formula (I′), or a stereoisomer or tautomer thereof, or a pharmaceutically acceptable salt of any of the foregoing, or any variation or embodiment thereof.

In some embodiments of the compound of Formula (I), Ring 3 is 3- to 6-membered cycloalkyl optionally substituted with one or more R^{3a}, 4- to 6-membered heterocyclyl optionally substituted with one or more R^{3a}, phenyl optionally substituted with one or more R^{3b}, or 5- to 6-membered heteroaryl optionally substituted with one or more R^{3b}, wherein each R^{3a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo, and each R^{3b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some embodiments, each R^{3a} is independently halo, methyl, trifluoromethyl, OH, OCH₃, acetyl, CN, or oxo. In some embodiments, each R^{3a} is oxo. In some embodiments, each R^{3b} is independently halo, methyl, trifluoromethyl, OH, OCH₃, acetyl, or CN. In some embodiments of the compound of Formula (I), Ring 3 is 4- to 6-membered heterocyclyl optionally substituted with one or more R^{3a}, phenyl optionally substituted with one or more R^{3b}, or 5- to 6-membered heteroaryl optionally substituted with one or more R^{3b}. In some embodiments of the compound of Formula (I), Ring 3 is 5- to 6-membered heterocyclyl optionally substituted with one or more R^{3a}, phenyl optionally substituted with one or more R^{3b}, or 5- to 6-membered heteroaryl optionally substituted with one or more R^{3b}. In some embodiments of the compound of Formula (I), Ring 3 is 5- to 6-membered heterocyclyl optionally substituted with one or more R^{3a}, phenyl optionally substituted with one or more R^{3b}, or 5-membered heteroaryl optionally substituted with one or more R^{3b}. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments, Ring 3 is 3- to 10-membered cycloalkyl optionally substituted with one or more R^{3a}, 4- to 10-membered heterocyclyl optionally substituted with one or more R^{3a}, 6- to 10-membered aryl optionally substituted with one or more R^{3b}, or 5- to 10-membered heteroaryl optionally substituted with one or more R^{3b}, wherein each R^{3a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-(3- to 6-membered cycloalkyl), C₁₋₄alkylene-N(R^{y})—C₁₋₄alkyl, C₁₋₄alkylene-S(O)ₙR^{x}, or oxo, and each R^{3b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, O-phenyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some embodiments, each R^{3a} is independently halo, methyl, trifluoromethyl, OH, OCH₃, acetyl, CN, or oxo. In some embodiments, each R^{3a} is oxo. In some embodiments, each R^{3a} is C(O)R^{w}. In some embodiments, each R^{3a} is C₁₋₄alkylene-(3- to 6-membered cycloalkyl). In some embodiments, each R^{3a} is methylene-(3- to 6-membered cycloalkyl). In some embodiments, each R^{3a} is methylene-(cyclopropyl). In some embodiments, each R^{3b} is independently halo, methyl, trifluoromethyl, OH, OCH₃, acetyl, or CN. In some embodiments, each R^{3b} is O-phenyl. In some embodiments, Ring 3 is 4- to 6-membered heterocyclyl optionally substituted with one or more R^{3a}, phenyl optionally substituted with one or more R^{3b}, or 5- to 6-membered heteroaryl optionally substituted with one or more R^{3b}. In some embodiments, Ring 3 is 5- to 6-membered heterocyclyl optionally substituted with one or more R^{3a}, phenyl optionally substituted with one or more R^{3b}, or 5- to 6-membered heteroaryl optionally substituted with one or more R^{3b}. In some embodiments, Ring 3 is 5- to 6-membered heterocyclyl optionally substituted with one or more R^{3a}, phenyl optionally substituted with one or more R^{3b}, or 5-membered heteroaryl optionally substituted with one or more R^{3b}. In some embodiments, Ring 3 is 6- to 10-membered heterocyclyl optionally substituted with one or more R^{3a}, naphthyl optionally substituted with one or more R^{3b}, or 6- to 10-membered heteroaryl optionally substituted with one or more R^{3b}. In some embodiments, Ring 3 is 9- to 10-membered heterocyclyl optionally substituted with one or more R^{3a}, naphthyl optionally substituted with one or more R^{3b}, or 9- to 10-membered heteroaryl optionally substituted with one or more R^{3b}. In some embodiments, Ring 3 is 9-membered heterocyclyl optionally substituted with one or more R^{3a}, naphthyl optionally substituted with one or more R^{3b}, or 9-membered heteroaryl optionally substituted with one or more R^{3b}. In some embodiments, Ring 3 is 10-membered heterocyclyl optionally substituted with one or more R^{3a}, naphthyl optionally substituted with one or more R^{3b}, or 10-membered heteroaryl optionally substituted with one or more R^{3b}.

In some embodiments of the compound of Formula (I), Ring 3 is 3- to 6-membered cycloalkyl optionally substituted with one or more R^{3a}. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments, Ring 3 is 3- to 10-membered cycloalkyl optionally substituted with one or more R^{3a}. In some embodiments, Ring 3 is 4- to 10-membered heterocyclyl optionally substituted with one or more R^{3a}. In some embodiments, Ring 3 is 6- to 10-membered aryl optionally substituted with one or more R^{3b}. In some embodiments, Ring 3 is 5- to 10-membered heteroaryl optionally substituted with one or more R^{3b}.

In some embodiments of the compound of Formula (I), Ring 3 is 4- to 6-membered heterocyclyl optionally substituted with one or more R^{3a}. In some embodiments of the compound of Formula (I), Ring 3 is 4- to 6-membered nitrogen-containing heterocyclyl optionally substituted with one or more R^{3a}. In some embodiments, Ring 3 is pyrrolidinyl, piperidinyl, piperazinyl, or morpholinyl. In some embodiments, Ring 3 is

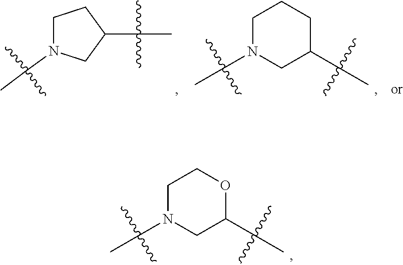 [SMILES: *C1CCN(*)C1 ; *C1CCCN(*)C1 ; *C1CN(*)CCO1]

each of which is optionally substituted with one or more R^{3a}. In some embodiments, Ring 3 is

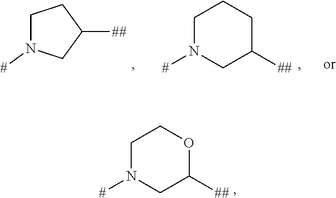

each of which is optionally substituted with one or more R^{3a}, wherein # indicates attachment to the pyrimidine ring of Formula (I), and ## indicates attachment to Ring 4, when present. In some embodiments, R^{3a} is oxo. In some embodiments, Ring 3 is

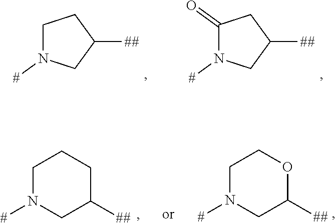

wherein # indicates attachment to the pyrimidine ring of Formula (I), and ## indicates attachment to Ring 4, when present. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments, Ring 3 is 4- to 10-membered heterocyclyl optionally substituted with one or more R^{3a}. In some embodiments, Ring 3 is

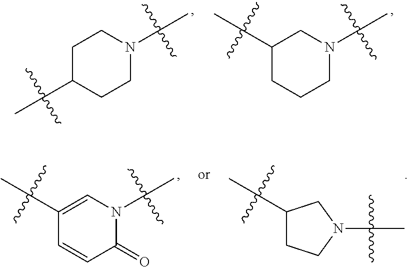 [SMILES: *C1CCN(*)CC1 ; *C1CCCN(*)C1 ; *c1ccc(=O)n(*)c1 ; *C1CCN(*)C1]

In some embodiments, Ring 3 is

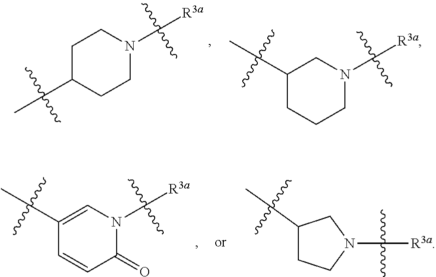

In some embodiments, Ring 3 is

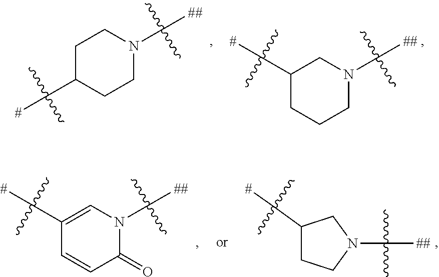

wherein # indicates attachment to the pyrimidine ring of Formula (I′), or any variation thereof, and ## indicates attachment to Ring 4, when present. In some embodiments, Ring 3 is 4- to 10-membered nitrogen-containing heterocyclyl optionally substituted with one or more R^{3a}. In some embodiments, Ring 3 is

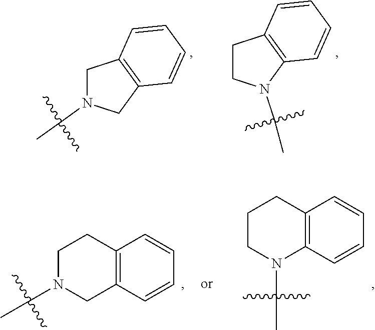 [SMILES: *N1Cc2ccccc2C1 ; *N1CCc2ccccc21 ; *N1CCc2ccccc2C1 ; *N1CCCc2ccccc21]

each of which is optionally substituted with one or more R^{3a}. In some embodiments, Ring 3 is

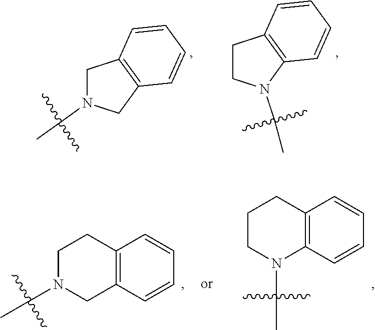 [SMILES: *N1Cc2ccccc2C1 ; *N1CCc2ccccc21 ; *N1CCc2ccccc2C1 ; *N1CCCc2ccccc21]

each of which unsubstituted.

In some embodiments of the compound of Formula (I), Ring 3 is phenyl optionally substituted with one or more R^{3b}. In some embodiments, Ring 3 is

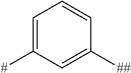

optionally substituted with one or more R^{3b}. In some embodiments, Ring 3 is

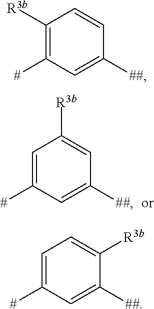

In some embodiments, each R^{3b} is independently halo, methyl, trifluoromethyl, OH, OCH₃, acetyl, or CN. In some embodiments, each R^{3b} is independently halo, methyl, or OCH₃. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Ring 3 is 5- to 6-membered heteroaryl optionally substituted with one or more R^{3b}. In some embodiments of the compound of Formula (I), Ring 3 is 5- to 6-membered nitrogen-containing heteroaryl optionally substituted with one or more R^{3b}. In some embodiments, Ring 3 is 5-membered heteroaryl optionally substituted with one or more R^{3b}. In some embodiments, Ring 3 is pyrazolyl optionally substituted with one or more R^{3b}. In some embodiments, Ring 3 is

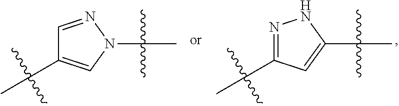 [SMILES: *c1cnn(*)c1 ; *c1cc(*)[nH]n1]

each of which is optionally substituted with one or more R^{3b}. In some embodiments, Ring 3 is

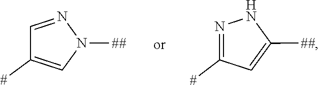

each of which is optionally substituted with one or more R^{3b}, wherein # indicates attachment to the pyrimidine ring of Formula (I), and ## indicates attachment to Ring 4, when present. In some embodiments, Ring 3 is

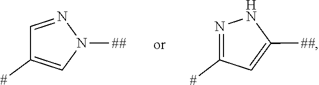

wherein # indicates attachment to the pyrimidine ring of Formula (I), and ## indicates attachment to Ring 4, when present. In some embodiments, Ring 3 is 6-membered heteroaryl optionally substituted with one or more R^{3b}. In some embodiments, Ring 3 is pyridinyl, pyrimidinyl, or pyrazinyl optionally substituted with one or more R^{3b}. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments, Ring 3 is

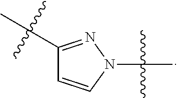

In some embodiments, Ring 3 is

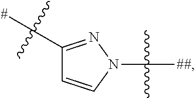

wherein # indicates attachment to the pyrimidine ring of Formula (I′) or any variation thereof, and ## indicates attachment to Ring 4, when present.

In some embodiments of the compound of Formula (I), Ring 4 is absent. In some embodiments of the compound of Formula (I), Ring 4 is present and is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b}, wherein each R^{4a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo, and each R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙRx. In some embodiments, each R^{4a} is independently halo, methyl, trifluoromethyl, OH, OCH₃, acetyl, CN, or oxo. In some embodiments, each R^{4a} is methyl or acetyl. In some embodiments, each R^{4b} is independently halo, methyl, trifluoromethyl, OH, OCH₃, acetyl, or CN. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments, each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-(3- to 6-membered cycloalkyl), C₁₋₄alkylene-N(R^{y})—C₁₋₄alkyl, C₁₋₄alkylene-S(O)ₙR^{x}, or oxo.

In some embodiments, Ring 4 is absent. In some embodiments, Ring 4 is present and is a ring selected from the group consisting of 3- to 10-membered cycloalkyl optionally substituted with one or more R^{4a}, 4- to 10-membered heterocyclyl optionally substituted with one or more R^{4a}, 6- to 10-membered aryl optionally substituted with one or more R^{4b}, and 5- to 10-membered heteroaryl optionally substituted with one or more R^{4b}, wherein each R^{4a} is independently halo, C₁₋₄alkyl, C₁₋₄haloalkyl, OH, C₁₋₄alkoxy, C₁₋₄haloalkoxy, C(O)C₁₋₄alkyl, C(O)R*, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-(3- to 6-membered cycloalkyl), C₁₋₄alkylene-N(R^{y})—C₁₋₄alkyl, C₁₋₄alkylene-S(O)ₙR^{x}, or oxo, and each R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, O-phenyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some embodiments, each R^{4a} is independently halo, methyl, trifluoromethyl, OH, OCH₃, acetyl, CN, or oxo. In some embodiments, each R^{4a} is methyl or acetyl. In some embodiments, each R^{4a} is C(O)R^{w}. In some embodiments, each R^{4a} is C₁₋₄alkylene-(3- to 6-membered cycloalkyl). In some embodiments, each R^{4a} is methylene-(3- to 6-membered cycloalkyl). In some embodiments, each R^{4a} is methylene-(cyclopropyl). In some embodiments, each R^{4b} is independently halo, methyl, trifluoromethyl, OH, OCH₃, acetyl, or CN. In some embodiments, each R^{3b} is O-phenyl.

In some embodiments, Ring 4 is 3- to 6-membered cycloalkyl optionally substituted with one or more R^{4a}. In some embodiments, Ring 4 is cyclopropyl, cyclobutyl, cyclopentyl, or cyclohexyl optionally substituted with one or more R^{4a}. In some embodiments, Ring 4 is cyclopropyl, cyclobutyl, cyclopentyl, or cyclohexyl.

In some embodiments, Ring 4 is 4- to 6-membered heterocyclyl optionally substituted with one or more R^{4a}. In some embodiments, Ring 4 is 4- to 6-membered nitrogen-containing heterocyclyl optionally substituted with one or more R^{4a}. In some embodiments, Ring 4 is pyrrolidinyl, piperidinyl, or piperazinyl, each of which is optionally substituted with one or more R^{4a}. In some embodiments, Ring 4 is pyrrolidinyl or piperidinyl, each of which is optionally substituted with one or more R^{4a}. In some embodiments, Ring 4 is

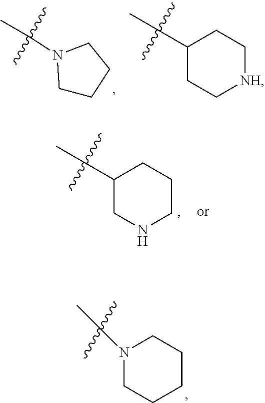 [SMILES: *N1CCCC1 ; *C1CCNCC1 ; *C1CCCNC1 ; *N1CCCCC1]

each of which is optionally substituted with one or more R^{4a}. In some embodiments, Ring 4 is

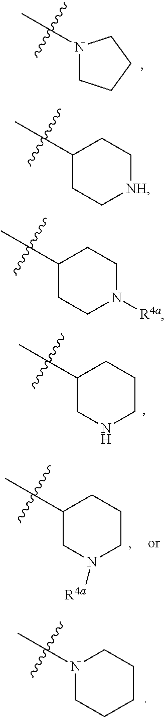

In some embodiments, each R^{4a} is independently halo, methyl, trifluoromethyl, OH, OCH₃, acetyl, CN, or oxo. In some embodiments, each R^{4a} is methyl or acetyl.

In some embodiments, Ring 4 is azetidinyl, oxetanyl, pyridonyl, each of which is optionally substituted with one or more R^{4a}. In some embodiments, Ring 4 is

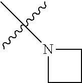 [SMILES: *N1CCC1]

optionally substituted with one or more R^{4a}. In some embodiments, Ring 4 is unsubstituted

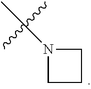 [SMILES: *N1CCC1]

In some embodiments, Ring 4 is

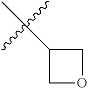 [SMILES: *C1COC1]

optionally substituted with one or more R^{4a}. In some embodiments, Ring 4 is unsubstituted

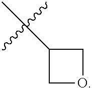 [SMILES: *C1COC1]

In some embodiments, Ring 4 is

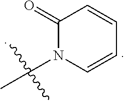 [SMILES: *n1ccccc1=O]

In some embodiments, Ring 4 is phenyl optionally substituted with one or more R^{4b}. In some embodiments, Ring 4 is

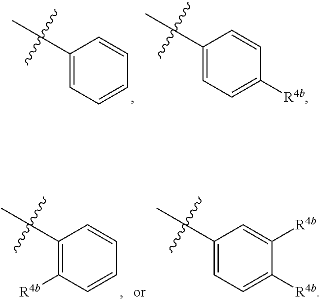

In some embodiments, each R^{4b} is independently halo, methyl, trifluoromethyl, OH, OCH₃, acetyl, or CN. In some embodiments, each R^{4b} is halo, methyl, or OCH₃.

In some embodiments, Ring 4 is phenyl optionally substituted with one or more R^{4b}. In some embodiments, Ring 4 is

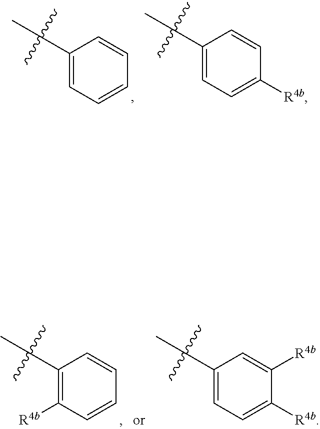

In some embodiments, each R^{4b} is independently halo, methyl, trifluoromethyl, OH, OCH₃, acetyl, O-phenyl, or CN. In some embodiments, each R^{4b} is halo, methyl, or OCH₃. In some embodiments, each R^{4b} is O-phenyl.

In some embodiments, Ring 4 is 5- to 6-membered heteroaryl optionally substituted with one or more R^{4b}. In some embodiments, Ring 4 is 5- to 6-membered nitrogen-containing heteroaryl optionally substituted with one or more R^{4b}. In some embodiments, Ring 4 is 6-membered heteroaryl optionally substituted with one or more R^{4b}. In some embodiments, Ring 4 is pyridinyl or pyrazinyl, each of which is optionally substituted with one or more R^{4b}. In some embodiments, Ring 4 is

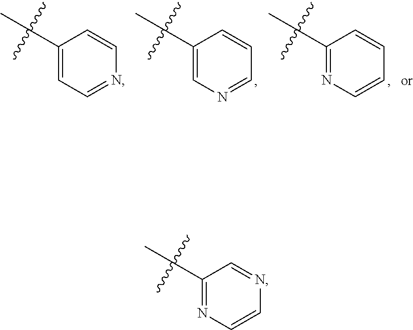 [SMILES: *c1ccncc1 ; *c1cccnc1 ; *c1ccccn1 ; *c1cnccn1]

each of which is optionally substituted with one or more R^{4b}. In some embodiments, Ring 4 is

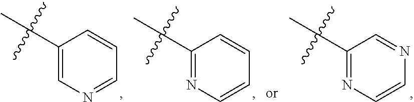 [SMILES: *c1cccnc1 ; *c1ccccn1 ; *c1cnccn1]

each of which is optionally substituted with one or more R^{4a}. In some embodiments, Ring 4 is

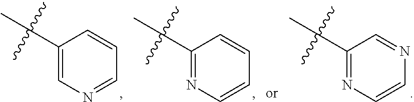 [SMILES: *c1cccnc1 ; *c1ccccn1 ; *c1cnccn1]

In some embodiments, Ring 4 is 3- to 10-membered cycloalkyl optionally substituted with one or more R^{4a}. In some embodiments, Ring 4 is 4- to 10-membered heterocyclyl optionally substituted with one or more R^{4a}. In some embodiments, Ring 4 is 6- to 10-membered aryl optionally substituted with one or more R^{4b}. In some embodiments, Ring 4 is 5- to 10-membered heteroaryl optionally substituted with one or more R^{4b}.

In some embodiments, each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, O-phenyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}.

In some embodiments, each R^{w} is independently 3- to 10-membered cycloalkyl, phenyl optionally substituted with halo, or 4- to 12-membered heterocyclyl optionally substituted with oxo. In some embodiments, each R^{w} is independently cyclopropyl, cyclobutyl, or cyclopentyl. In some embodiments, each R^{w} is phenyl optionally substituted with halo. In some embodiments, each R^{w} is phenyl optionally substituted with fluoro or chloro. In some embodiments, each R^{w} is phenyl optionally substituted with fluoro. In some embodiments, each R^{w} is independently 4- to 12-membered heterocyclyl optionally substituted with oxo. In some embodiments, each R^{w} is oxetanyl. In some embodiments, each R^{w} is

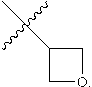 [SMILES: *C1COC1]

In some embodiments, each R^{w} is pyridonyl. In some embodiments, each R^{w} is

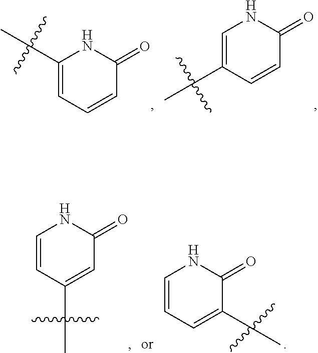 [SMILES: *c1cccc(=O)[nH]1 ; *c1ccc(=O)[nH]c1 ; *c1cc[nH]c(=O)c1 ; *c1ccc[nH]c1=O]

In some embodiments, Ring 4 is absent and Q is of Formula (i). In some embodiments, Ring 4 is present and Q is of Formula (i).

In some embodiments, Ring 4 is absent and Q is Ring 5. In some embodiments, Ring 4 is present and Q is Ring 5. In some embodiments, Ring 4 is present and Q is Ring 5, provided that when Ring 3 and Ring 4 are each unsubstituted phenyl, Ring 5 is substituted with one or more R⁵.

In some embodiments of the compound of Formula (I), Q is Ring 5, and the compound is of Formula (II):

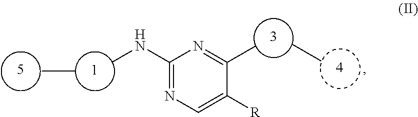

or a tautomer or stereoisomer thereof, or a pharmaceutically acceptable salt of any of the foregoing, wherein R, Ring 1, Ring 3, Ring 4, and Ring 5 are as detailed herein for Formula (I). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (II), Ring 5 is a pyrrolidinone or imidazolidinone, each of which is optionally substituted with one or more R⁵, wherein each R⁵ is independently C₁₋₄alkyl, or two R⁵ are taken together with the atom(s) to which they are attached to form a four- to six-membered ring which is optionally substituted with C₁₋₄alkyl or C(O)C₁₋₄alkyl. In some embodiments, Ring 5 is pyrrolidinone optionally substituted with one or more R⁵. In some embodiments, Ring 5 is

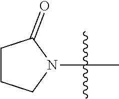 [SMILES: *N1CCCC1=O]

optionally substituted with one or more R⁵. In some embodiments, two R⁵ are taken together with the atom(s) to which they are attached to form a four- to six-membered ring which is optionally substituted with C₁₋₄alkyl or C(O)C₁₋₄alkyl. In some embodiments, two R⁵ on the same carbon atom are taken together with the atom to which they are attached to form a four- to six-membered ring which is optionally substituted with C₁₋₄alkyl or C(O)C₁₋₄alkyl. In some embodiments, Ring 5 is

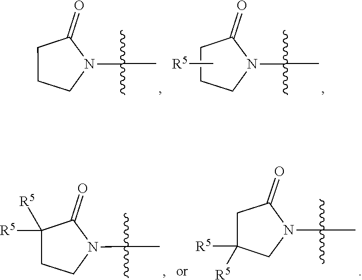

In some embodiments, Ring 5 is

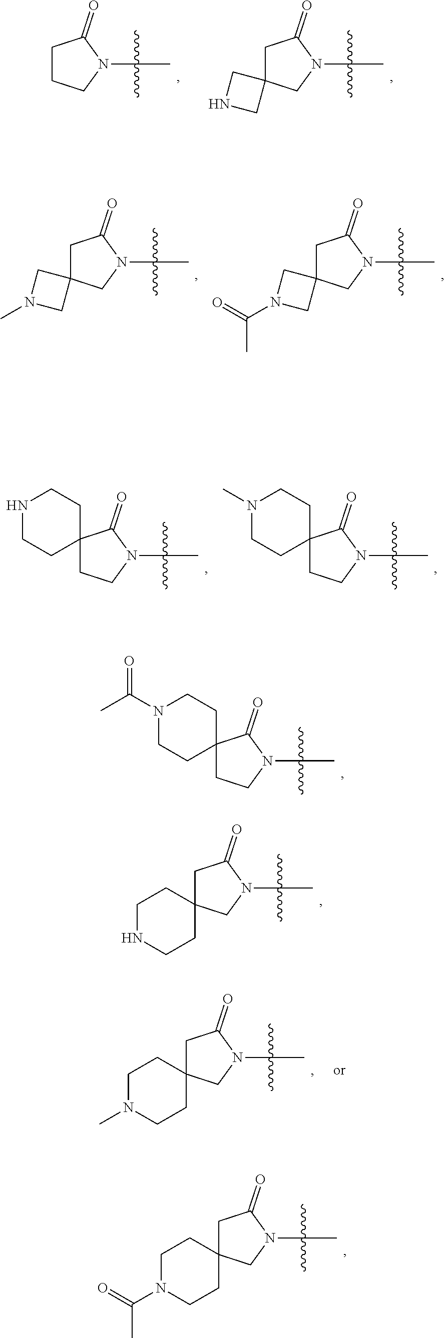

In some embodiments of the compound of Formula (I), Q is of Formula (i), and the compound is of Formula (III):

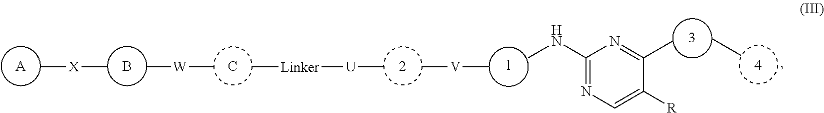

or a tautomer or stereoisomer thereof, or a pharmaceutically acceptable salt of any of the foregoing, wherein Ring A, X, Ring B, W, Ring C, Linker, U, Ring 2, V, Ring 1, Ring 3, and Ring 4 are as detailed herein for Formula (I). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), Ring A is selected from the group consisting of

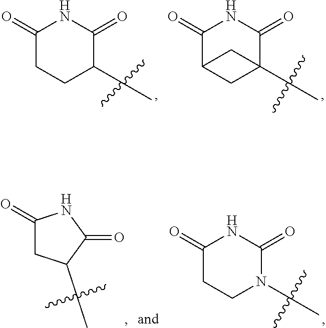 [SMILES: *C1CCC(=O)NC1=O ; *C12CC(C1)C(=O)NC2=O ; *C1CC(=O)NC1=O ; *N1CCC(=O)NC1=O]

each of which is optionally substituted with C₁₋₄alkyl or halo. In some embodiments, Ring A is

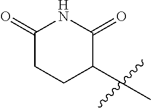 [SMILES: *C1CCC(=O)NC1=O]

optionally substituted with C₁₋₄alkyl or halo. In some embodiments, Ring A is

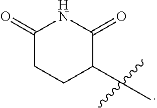 [SMILES: *C1CCC(=O)NC1=O]

In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), X is a bond, —O—, —NH—, or —NHC(O)—. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), Ring B is selected from the group consisting of

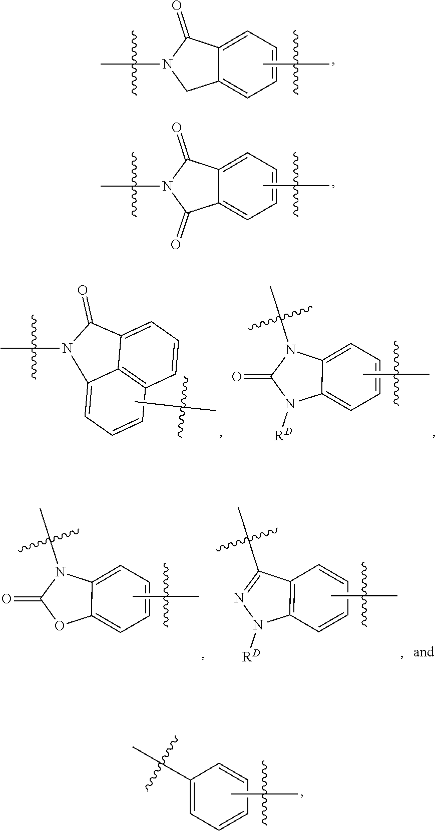

each of which is optionally substituted with one or more halo or OH, wherein R^{D} is H or C₁₋₄alkyl. In some embodiments, Ring B is selected from the group consisting of

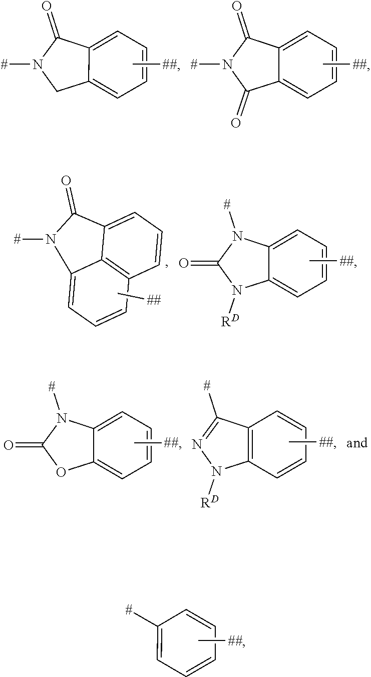

each of which is optionally substituted with one or more halo or OH, wherein R^{D} is H or C₁₋₄alkyl, # indicates attachment to X, and ## indicates attachment to W. In some embodiments, Ring B is

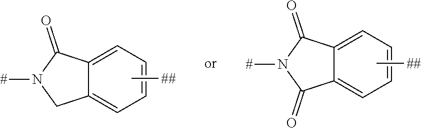

optionally substituted with one or more halo or OH. In some embodiments, Ring B is

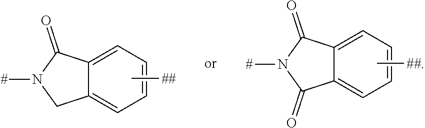

In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), W is a bond, —O—, —NH—, or —NHC(O)—. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), Ring C is absent. In some embodiments, Ring C is present and is 4- to 12-membered heterocyclyl optionally substituted with one or more R^{C}, wherein each R^{C} is independently halo, OH, C₁₋₄alkyl, or C₁₋₄haloalkyl. In some embodiments, Ring C is 4- to 12-membered nitrogen-containing heterocyclyl optionally substituted with one or more R^{C}. In some embodiments, Ring C is 5- to 6-membered heterocyclyl optionally substituted with one or more R^{C}. In some embodiments, Ring C is

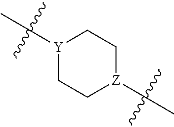

optionally substituted with one or more R^{C}, wherein Y and Z are independently CH or N. In some embodiments, Ring C is piperidinyl or piperazinyl, each of which is optionally substituted with one or more R^{C}. In some embodiments, Ring C is piperidinyl optionally substituted with one or more R^{C}. In some embodiments, Ring C is piperazinyl optionally substituted with one or more R^{C}. In some embodiments, Ring C is

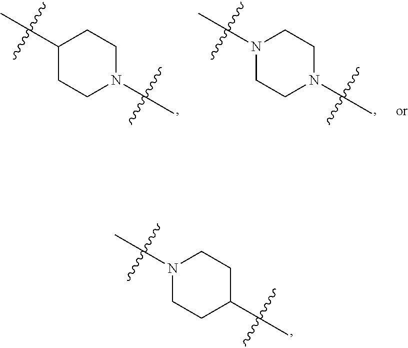 [SMILES: *C1CCN(*)CC1 ; *N1CCN(*)CC1 ; *C1CCN(*)CC1]

each of which is optionally substituted with one or more R^{C}. In some embodiments, Ring C is

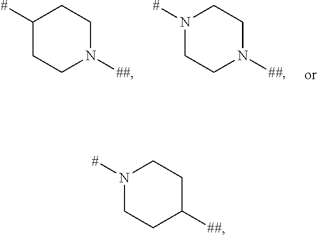

each of which is optionally substituted with one or more R^{C}, wherein # indicates attachment to W and ## indicates attachment to Linker. In some embodiments, Ring C is

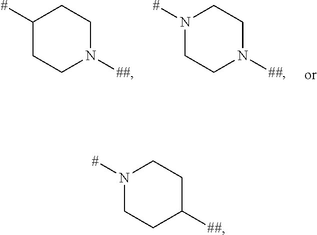

wherein # indicates attachment to W and ## indicates attachment to Linker. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein * indicates attachment to Ring C when Ring C is present, or attachment to W when Ring C is absent; and ** indicates attachment to U when Ring 2 is present, or attachment to V when Ring 2 is absent; L¹ is a bond or C₁₋₆ alkylene; L² is a 3- to 10-membered cycloalkyl or 4- to 12-membered heterocyclyl optionally substituted with one or more R^{L}, wherein each R^{L} is independently halo, OH, C₁₋₄alkyl, or C₁₋₄haloalkyl; and L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y}). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), L is a bond, C₁₋₁₀alkylene, C(O)—C₁₋₁₀alkylene, C₁₋₆alkylene-C(O)—C₁₋₁₀alkylene, Co-alkylene-C(O)N(R^{y})—C₁₋₁₀alkylene, or is of the formula *-L¹-L²-L³-**, wherein L¹ is a bond or C₁₋₆ alkylene, L² is a piperidinyl ring or piperazinyl ring, and L³ is C₁₋₁₀ alkylene. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) or (III), Ring 2 is absent. In some embodiments, Ring 2 is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b}, wherein each R^{2a} a is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo; and each R^{2b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x}. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments, Ring 2 is 4- to 12-membered heterocyclyl optionally substituted with one or more R^{2a}. In some embodiments, Ring 2 is 4- to 12-membered nitrogen-containing heterocyclyl optionally substituted with one or more R^{2a}. In some embodiments, Ring 2 is

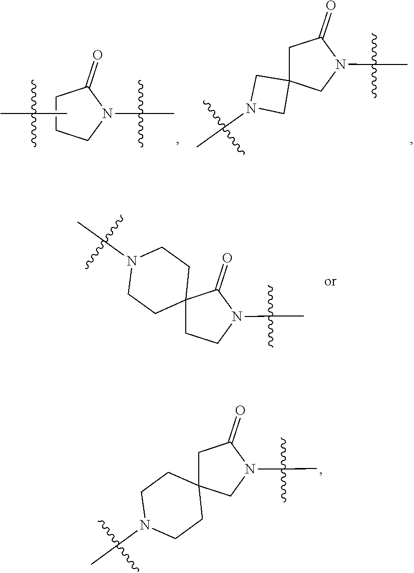

each of which is optionally substituted with one or more R^{2a}. In some embodiments, Ring 2 is

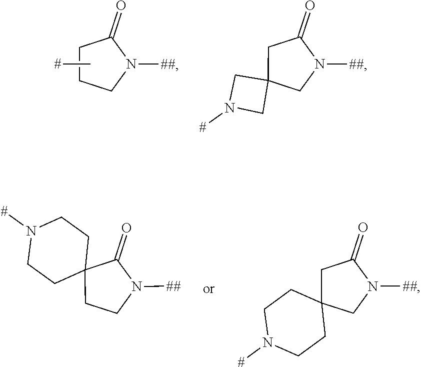

each of which is optionally substituted with one or more R^{2a}, wherein # indicates attachment to U and ** indicates attachment to V. In some embodiments, Ring 2 is

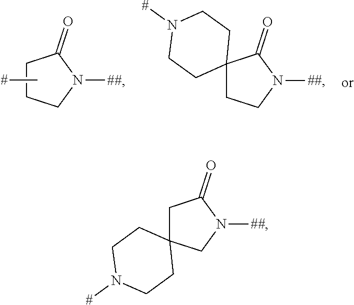

optionally substituted with one or more R^{2a}. In some embodiments, Ring 2 is

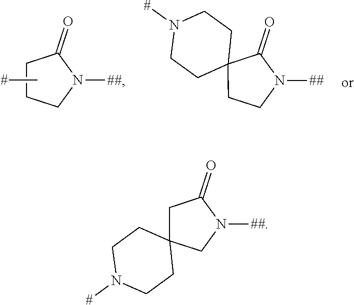

In some embodiments, V is a bond, —C(O)—, —C(O)NH—, or —NHC(O)—. In some embodiments, V is a bond, —C(O)—, #—C(O)NH—##, or #—NHC(O)—##, wherein: # indicates attachment to Ring 2 when Ring 2 is present or # indicates attachment to Linker when Ring 2 is absent; and ## indicates attachment to Ring 1. In some embodiments, V is a bond, —C(O)—, or #—C(O)NH—##. In some embodiments, V is a bond or #—C(O)NH—##.

In some embodiments, each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl; each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and each n is independently 0, 1, or 2.

It is to be understood that all embodiments of Formula (I) and sub-formulas thereof, also apply to Formulas (II) and (III), including all sub-formulas such as (II-a), (II-b), (II-c), (II-d), (II-e), (II-f), (II-g), (II-h), (III-a), (III-b), (III-c), (III-d), and (III-e), and variations thereof, as applicable. Further, the present disclosure contemplates combinations of features as disclosed herein, as if each were individually listed. Exemplary combinations of features are provided herein, but are not to be construed as limiting.

It is to be understood that all embodiments of Formula (I) and sub-formulas thereof, also apply to Formulas (I′), (II), and (III), including all sub-formulas such as (II-a), (II-b), (II-c), (II-d), (II-e), (II-f), (II-g), (II-h), (III-a), (III-b), (III-c), (III-d), and (III-e), and variations thereof, as applicable. Further, the present disclosure contemplates combinations of features as disclosed herein, as if each were individually listed. Exemplary combinations of features are provided herein, but are not to be construed as limiting.

In some embodiments of the compound of Formula (I), Ring 4 is present and the compound is of Formula (I-a),

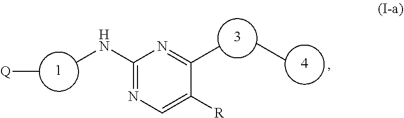

or a tautomer or stereoisomer thereof, or a pharmaceutically acceptable salt of any of the foregoing, wherein Q, R, Ring 1, Ring 3, and Ring 4 are as described herein for Formula (I). In some embodiments, Ring 1 is

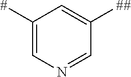

optionally substituted with one or more R¹, wherein # indicates attachment to Q and ## indicates attachment to the aminopyrimidine of the compound of Formula (I-a). In some embodiments, Ring 3 is

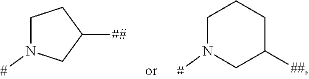

each of which is optionally substituted with one or more R^{3a}, wherein # indicates attachment to the pyrimidine ring of Formula (I-a), and ## indicates attachment to Ring 4. In some embodiments, Ring 3 is

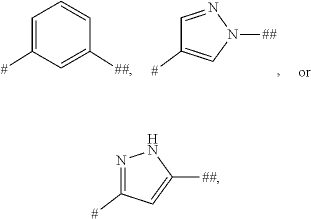

each of which is optionally substituted with one or more R^{3b}, wherein # indicates attachment to the pyrimidine ring of Formula (I-a), and ## indicates attachment to Ring 4. In some embodiments, either Ring 3 is phenyl optionally substituted with one or more R^{3b}, or Ring 4 is phenyl optionally substituted with one or more ^{4b}. In some embodiments, both Ring 3 is phenyl optionally substituted with one or more R^{3b}, and Ring 4 is phenyl optionally substituted with one or more ^{4b}. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Ring 3 is phenyl optionally substituted with one or more R^{3b}, and the compound is of Formula (I-b),

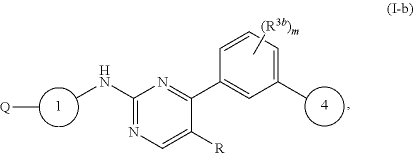

or a tautomer or stereoisomer thereof, or a pharmaceutically acceptable salt of any of the foregoing, wherein m may be an integer from 0-4, and Q, R, Ring 1, and Ring 4 are as described herein for Formula (I). In some embodiments, Ring 1 is

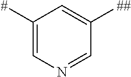

optionally substituted with one or more R¹, wherein # indicates attachment to Q and ## indicates attachment to the aminopyrimidine of the compound of Formula (I-b). In some embodiments, Ring 4 is phenyl optionally substituted with one or more R^{4b}. In some embodiments, Ring 4 is not phenyl optionally substituted with one or more R^{4b}. In some embodiments, Ring 4 is a monocyclic ring selected from the group consisting of 3- to 6-membered cycloalkyl optionally substituted with one or more R^{4a}, 4- to 6-membered heterocyclyl optionally substituted with one or more R^{4a}, and 5- to 6-membered heteroaryl optionally substituted with one or more R^{4b}. In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Ring 4 is phenyl optionally substituted with one or more R^{4b}, and the compound is of Formula (I-c),

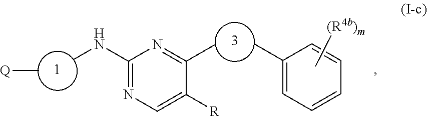

or a tautomer or stereoisomer thereof, or a pharmaceutically acceptable salt of any of the foregoing, wherein m may be an integer from 0-4, and Q, R, Ring 1, and Ring 3 are as described herein for Formula (I). In some embodiments, Ring 1 is

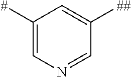

optionally substituted with one or more R¹, wherein # indicates attachment to Q and ## indicates attachment to the aminopyrimidine of the compound of Formula (I-c). In some embodiments, Ring 3 is

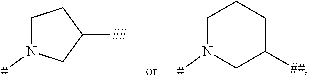

each of which is optionally substituted with one or more R^{3a}, wherein # indicates attachment to the pyrimidine ring of Formula (I-c), and ## indicates attachment to Ring 4. In some embodiments, Ring 3 is

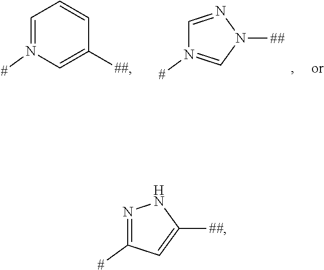

each of which is optionally substituted with one or more R^{3b}, wherein # indicates attachment to the pyrimidine ring of Formula (I-c), and ## indicates attachment to Ring 4. In some embodiments, Ring 3 is phenyl optionally substituted with one or more R^{3b}. In some embodiments, Ring 3 is not phenyl optionally substituted with one or more R^{3b}. In some embodiments, Ring 3 is

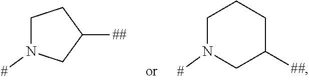

each of which is optionally substituted with one or more R^{3a}, or Ring 3 is

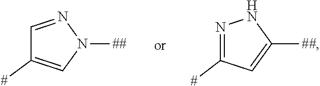

each of which is optionally substituted with one or more R^{3b}, wherein # indicates attachment to the pyrimidine ring of Formula (I-c). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Ring 3 is phenyl optionally substituted with one or more R^{3b}, Ring 4 is phenyl optionally substituted with one or more R^{4b}, and the compound is of Formula (I-d),

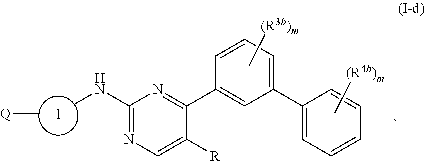

or a tautomer or stereoisomer thereof, or a pharmaceutically acceptable salt of any of the foregoing, wherein each m is an integer from 0-4, and Q, R, and Ring 1 are as described herein for Formula (I), provided that, when Ring 3 and Ring 4 are each unsubstituted phenyl and Q is Ring 5, Ring 5 is substituted with one or more R⁵. In some embodiments, Ring 1 is

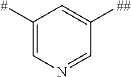

optionally substituted with one or more R¹, wherein # indicates attachment to Q and ## indicates attachment to the aminopyrimidine of the compound of Formula (I-d). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Ring 3 is phenyl, Ring 4 is phenyl, and the compound is of Formula (I-e),

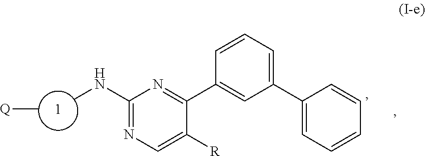

or a tautomer or stereoisomer thereof, or a pharmaceutically acceptable salt of any of the foregoing, wherein Q, R, and Ring 1 are as described herein for Formula (I), provided that, when Q is Ring 5, Ring 5 is substituted with one or more R⁵. In some embodiments, Ring 1 is

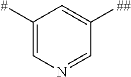

optionally substituted with one or more R¹, wherein # indicates attachment to Q and ## indicates attachment to the aminopyrimidine of the compound of Formula (I-e). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I), Ring 4 is absent and the compound is of Formula (I-f),

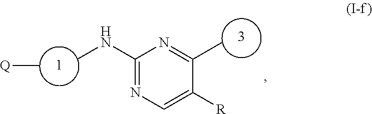

or a tautomer or stereoisomer thereof, or a pharmaceutically acceptable salt of any of the foregoing, wherein Q, R, Ring 1, and Ring 3 are as described herein for Formula (I). In some embodiments, Ring 1 is

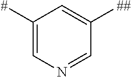

optionally substituted with one or more R¹, wherein # indicates attachment to Q and ## indicates attachment to the aminopyrimidine of the compound of Formula (I-f). In some embodiments, Ring 3 is

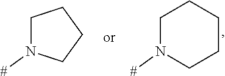

each of which is optionally substituted with one or more R^{3a}, wherein # indicates attachment to the pyrimidine ring of Formula (I-f). In some embodiments, Ring 3 is

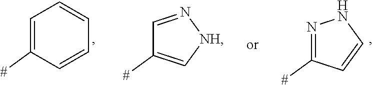

each of which is optionally substituted with one or more R^{3b}, wherein # indicates attachment to the pyrimidine ring of Formula (I-f). In some embodiments, Ring 3 is phenyl optionally substituted with one or more R^{3b}. In some embodiments, Ring 3 is not phenyl optionally substituted with one or more R^{3b}. In some embodiments, Ring 3 is

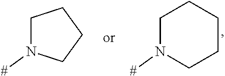

each of which is optionally substituted with one or more R^{3a}, or Ring 3 is

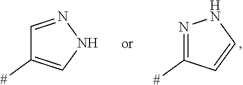

each of which is optionally substituted with one or more R^{3b}, wherein # indicates attachment to the pyrimidine ring of Formula (I-f). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (II), Q is Ring 5 and Ring 4 is present, and the compound is of Formula (II-a),

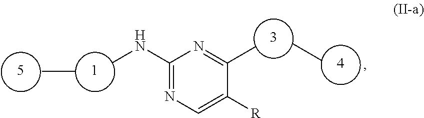

or a tautomer or stereoisomer thereof, or a pharmaceutically acceptable salt of any of the foregoing, wherein R, Ring 1, Ring 3, Ring 4, and Ring 5 are as described herein for Formula (I) and (II). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (II), Ring 5 is pyrrolidinone. In some embodiments, the compound is of Formula (II-b),

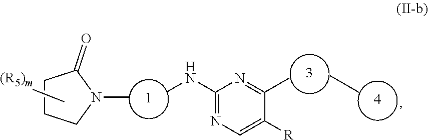

or a tautomer or stereoisomer thereof, or a pharmaceutically acceptable salt of any of the foregoing, wherein m is an integer from 0-6, and R, Ring 1, Ring 3, Ring 4, and R⁵ are as described herein for Formula (I) and (II). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (II), Ring 5 is imidazolidinone. In some embodiments, the compound is of Formula (II-c),

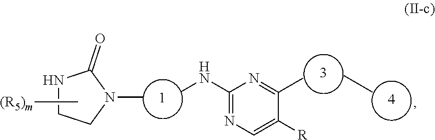

or a tautomer or stereoisomer thereof, or a pharmaceutically acceptable salt of any of the foregoing wherein m is an integer from 0-5, and R, Ring 1, Ring 3, Ring 4, and R⁵ are as described herein for Formula (I) and (II). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (II), Ring 5 is pyrrolidinone substituted with two R⁵, and is of formula (II-d) or (II-e),

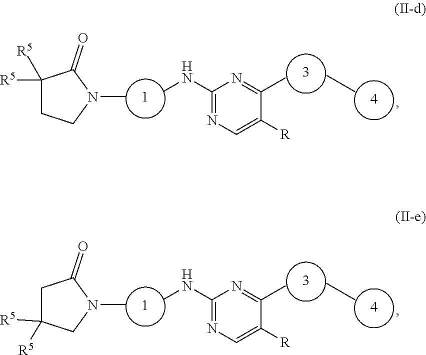

or a tautomer or stereoisomer thereof, or a pharmaceutically acceptable salt of any of the foregoing, wherein two R⁵ are taken together with the atom to which they are attached to form a four- to six-membered ring which is optionally substituted with C₁₋₄alkyl or C(O)C₁₋₄alkyl, and R, Ring 1, Ring 3, and Ring 4 are as described herein for Formula (I) and (II). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compounds of Formula (II-a), (II-b), (II-c), (II-d), (II-e), and (II-f), Ring 1 is

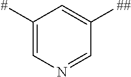

optionally substituted with one or more R¹, wherein # indicates attachment to the pyrrolidinone and ## indicates attachment to the aminopyrimidine of the compound of Formula (II-a), (II-b), (II-c), (II-d), and (II-e). In some embodiments, Ring 3 is

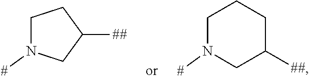

each of which is optionally substituted with one or more R^{3a}, wherein # indicates attachment to the pyrimidine ring of Formula (II-a), (II-b), (II-c), (II-d), and (II-e), and ## indicates attachment to Ring 4. In some embodiments, Ring 3 is

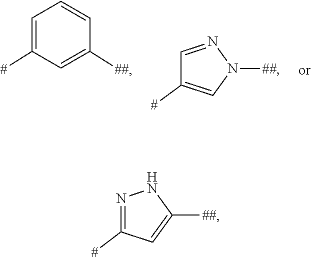

each of which is optionally substituted with one or more R^{3b}, wherein # indicates attachment to the pyrimidine ring of Formula (II-a), (II-b), (II-c), (II-d), and (II-e), and ## indicates attachment to Ring 4. In some embodiments, Ring 3 is phenyl optionally substituted with one or more R^{3b}. In some embodiments, Ring 3 is not phenyl optionally substituted with one or more R^{3b}. In some embodiments, Ring 3 is

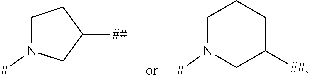

each of which is optionally substituted with one or more R^{3a}, or Ring 3 is

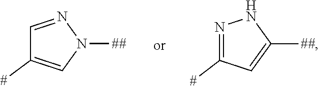

each of which is optionally substituted with one or more R^{3b}, wherein # indicates attachment to the pyrimidine ring of Formula (II-a), (II-b), (II-c), (II-d), and (II-e). In some embodiments, Ring 4 is phenyl optionally substituted with one or more R^{4b}. In some embodiments, Ring 4 is not phenyl optionally substituted with one or more R^{4b}. In some embodiments, Ring 4 is a monocyclic ring selected from the group consisting of 3- to 6-membered cycloalkyl optionally substituted with one or more R^{4a}, 4- to 6-membered heterocyclyl optionally substituted with one or more R^{4a}, and 5- to 6-membered heteroaryl optionally substituted with one or more R^{4b}.

In some embodiments of the compound of Formula (I and (II), Q is Ring 5 and Ring 4 is absent, and the compound is of Formula (II-f),

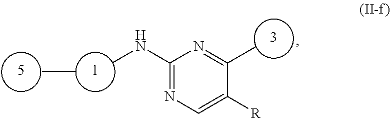

or a tautomer or stereoisomer thereof, or a pharmaceutically acceptable salt of any of the foregoing, wherein R, Ring 1, Ring 3, Ring 4, and Ring 5 are as described herein for Formula (I) and (II). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (II), Ring 5 is pyrrolidinone substituted with two R⁵, and is of formula (II-g) or (II-h),

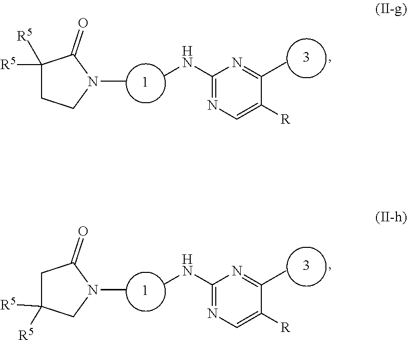

or a tautomer or stereoisomer thereof, or a pharmaceutically acceptable salt of any of the foregoing, wherein two R⁵ are taken together with the atom to which they are attached to form a four- to six-membered ring which is optionally substituted with C₁₋₄alkyl or C(O)C₁₋₄alkyl, and R, Ring 1, and Ring 3 are as described herein for Formula (I) and (II). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (II-f), (II-g), or (II-h), Ring 1 is

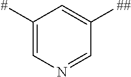

optionally substituted with one or more R¹, wherein # indicates attachment to the pyrrolidinone and ## indicates attachment to the aminopyrimidine of the compound of Formula (II-f), (II-g), or (II-h). In some embodiments, Ring 3 is

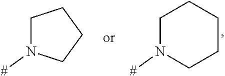

each of which is optionally substituted with one or more R^{3a}, wherein # indicates attachment to the pyrimidine ring of Formula (II-f), (II-g), or (II-h). In some embodiments, Ring 3 is

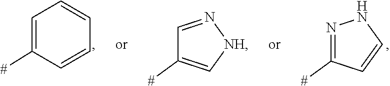

each of which is optionally substituted with one or more R^{3b}, wherein # indicates attachment to the pyrimidine ring of Formula (II-f), (II-g), or (II-h). In some embodiments, Ring 3 is phenyl optionally substituted with one or more R^{3b}. In some embodiments, Ring 3 is phenyl optionally substituted with one or more R^{3b}. In some embodiments, Ring 3 is not phenyl optionally substituted with one or more R^{3b}. In some embodiments, Ring 3 is

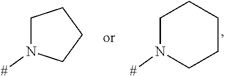

each of which is optionally substituted with one or more R^{3a}, or Ring 3 is

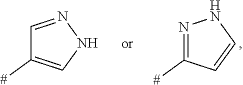

each of which is optionally substituted with one or more R^{3b}, wherein # indicates attachment to the pyrimidine ring of Formula (II-f), (II-g), or (II-h).

In some embodiments of the compound of Formula (I) and (III), Q is of Formula (i) and Ring C, Ring 2, and Ring 4 are each present, and the compound is of Formula (III-a),

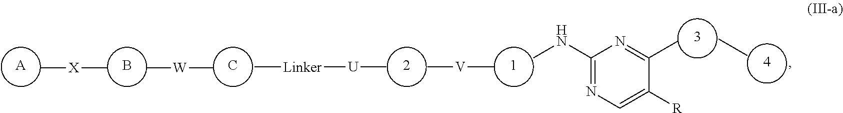

or a tautomer or stereoisomer thereof, or a pharmaceutically acceptable salt of any of the foregoing, wherein Ring A, X, Ring B, W, Ring C, Linker, U, Ring 2, V, R, Ring 1, Ring 3, and Ring 4 are as described herein for Formula (I) and (III). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (III), Q is of Formula (i) and Ring C is absent, and the compound is of Formula (III-b),

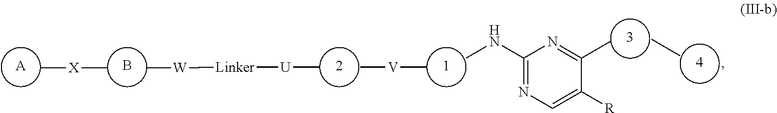

wherein Ring A, X, Ring B, W, Linker, U, Ring 2, V, R, Ring 1, Ring 3, and Ring 4 are as described herein for Formula (I) and (III). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (III), Q is of Formula (i) and Ring C and Ring 2 are absent, and the compound is of Formula (III-c),

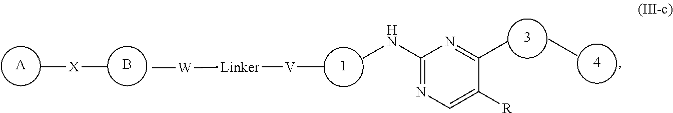

wherein Ring A, X, Ring B, W, Linker, V, R, Ring 1, Ring 3, and Ring 4 are as described herein for Formula (I) and (III). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (III-a), (III-b), and (III-c), Ring 1 is

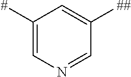

optionally substituted with one or more R¹, wherein # indicates attachment to V and ## indicates attachment to the aminopyrimidine of the compound of Formula (III-a), (III-b), and (III-c). In some embodiments, Ring 3 is

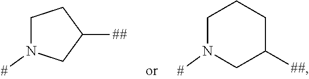

each of which is optionally substituted with one or more R^{3a}, wherein # indicates attachment to the pyrimidine ring of Formula (III-a), (III-b), and (III-c), and ## indicates attachment to Ring 4. In some embodiments, Ring 3 is

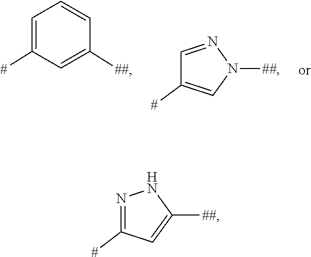

each of which is optionally substituted with one or more R^{3b}, wherein # indicates attachment to the pyrimidine ring of Formula (III-a), (III-b), and (III-c), and ## indicates attachment to Ring 4. In some embodiments, Ring 3 is phenyl optionally substituted with one or more R^{3b}. In some embodiments, Ring 3 is not phenyl optionally substituted with one or more R^{3b}. In some embodiments, Ring 3 is

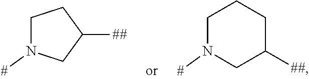

each of which is optionally substituted with one or more R^{3a}, or Ring 3 is

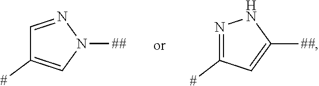

each of which is optionally substituted with one or more R^{3b}, wherein # indicates attachment to the pyrimidine ring of Formula (III-a), (III-b), and (III-c). In some embodiments, Ring 4 is phenyl optionally substituted with one or more R^{4b}. In some embodiments, Ring 4 is not phenyl optionally substituted with one or more R^{4b}. In some embodiments, Ring 4 is a monocyclic ring selected from the group consisting of 3- to 6-membered cycloalkyl optionally substituted with one or more R^{4a}, 4- to 6-membered heterocyclyl optionally substituted with one or more R^{4a}, and 5- to 6-membered heteroaryl optionally substituted with one or more R^{4b}.

In some embodiments of the compound of Formula (I) and (III), Q is of Formula (i) and Ring C and Ring 4 are each absent, and the compound is of Formula (III-d),

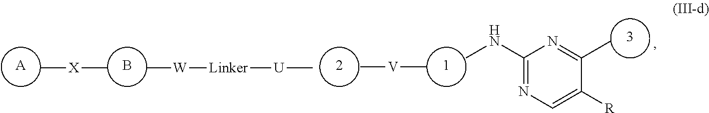

wherein Ring A, X, Ring B, W, Linker, U, Ring 2, V, R, Ring 1, and Ring 3 are as described herein for Formula (I) and (III). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (I) and (III), Q is of Formula (i) and Ring C, Ring 2, and Ring 4 are each absent, and the compound is of Formula (III-e),

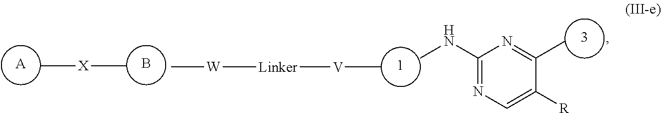

wherein Ring A, X, Ring B, W, Linker, V, R, Ring 1, and Ring 3 are as described herein for Formula (I) and (III). In some variations, the embodiments provided herein also apply to a compound of Formula (I′), or a stereoisomer or tautomer thereof, or a pharmaceutically acceptable salt of any of the foregoing, or any variation or embodiment thereof.

In some embodiments of the compound of Formula (III-d) and (III-e), Ring 1 is

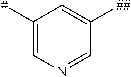

optionally substituted with one or more R¹, wherein # indicates attachment to V and ## indicates attachment to the aminopyrimidine of the compound of Formula (III-d) and (III-e). In some embodiments, Ring 3 is

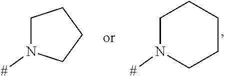

each of which is optionally substituted with one or more R^{3a}, wherein # indicates attachment to the pyrimidine ring of Formula (III-d) and (III-e). In some embodiments, Ring 3 is

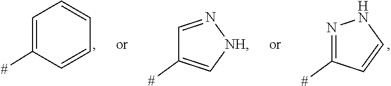

each of which is optionally substituted with one or more R^{3b}, wherein # indicates attachment to the pyrimidine ring of Formula (III-d) and (III-e). In some embodiments, Ring 3 is phenyl optionally substituted with one or more R^{3b}. In some embodiments, Ring 3 is phenyl optionally substituted with one or more R^{3b}. In some embodiments, Ring 3 is not phenyl optionally substituted with one or more R^{3b}. In some embodiments, Ring 3 is

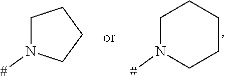

each of which is optionally substituted with one or more R^{3a}, or Ring 3 is

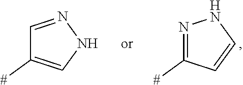

each of which is optionally substituted with one or more R^{3b}, wherein # indicates attachment to the pyrimidine ring of Formula (III-d) and (III-e).

In some embodiments of the foregoing formulae, including Formula (I), (III), and related formulas (III-a), (III-b), (III-c), (III-d), and (III-e), Ring A, X, Ring B, W, and C together are:

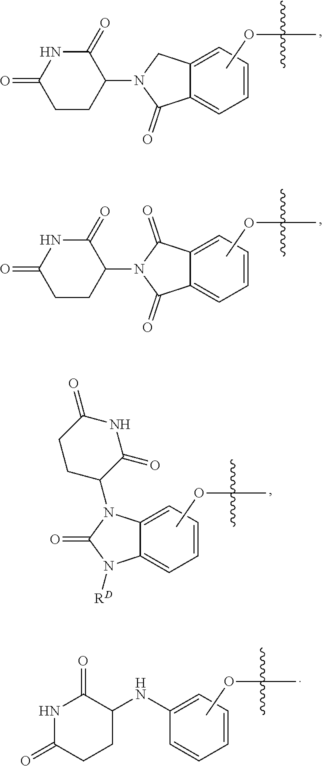

In some embodiments of the foregoing formulae, including Formula (I), (III), and related formulae, Ring A, X, Ring B, W, and C together are:

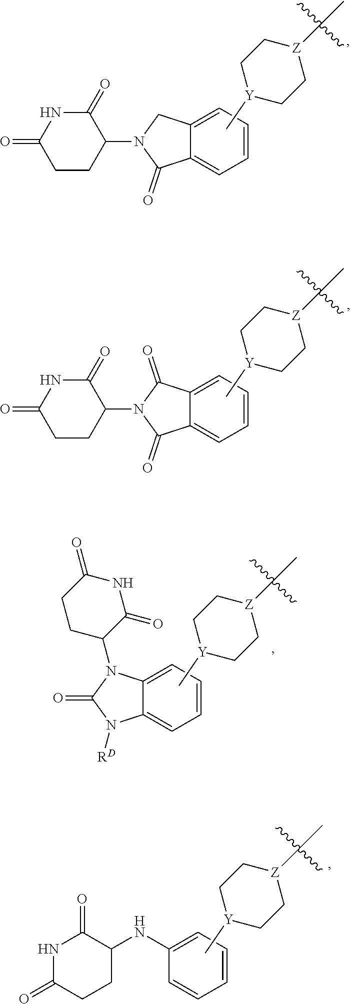

each of which is optionally substituted with one or more R^{C}, wherein Y and Z are independently CH or N. In some variations, the embodiments provided herein also apply to a compound of Formula (I′), or a stereoisomer or tautomer thereof, or a pharmaceutically acceptable salt of any of the foregoing, or any variation or embodiment thereof.

Representative compounds are listed in Tables 1 and 2.

In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1 and Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 1. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from Table 2.

In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-22, 24, 26-30, 32-50, 90, and 92-119 of Table 1, and compounds 51-56, 58-89, and 91 of Table 2. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 1-22, 24, 26-30, 32-50, 90, and 92-119 of Table 1. In some embodiments of a compound of Formula (I′) or (I), or any variation or embodiment thereof, or a stereoisomer or tautomer thereof, or a pharmaceutically acceptable salt of any of the foregoing, the compound, or a stereoisomer or tautomer thereof, or a pharmaceutically acceptable salt of any of the foregoing, is selected from compounds 51-56, 58-89, and 91 of Table 2.

TABLE 1
Representative Compounds of Formula (I′), (I), and (II)

| No. | Structure |
|---|---|
| 1 | 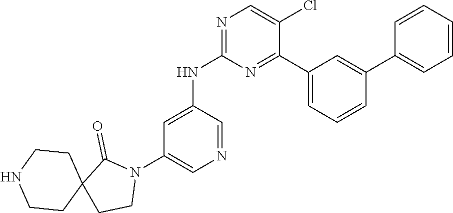 [SMILES: O=C1N(c2cncc(Nc3ncc(Cl)c(-c4cccc(-c5ccccc5)c4)n3)c2)CCC12CCNCC2] |
| 2 | 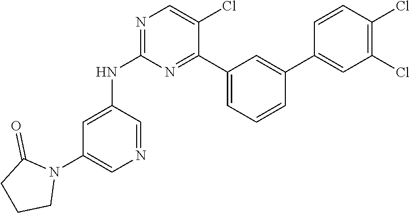 [SMILES: O=C1CCCN1c1cncc(Nc2ncc(Cl)c(-c3cccc(-c4ccc(Cl)c(Cl)c4)c3)n2)c1] |
| 3 | 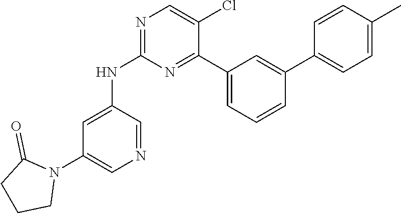 [SMILES: Cc1ccc(-c2cccc(-c3nc(Nc4cncc(N5CCCC5=O)c4)ncc3Cl)c2)cc1] |
| 4 | 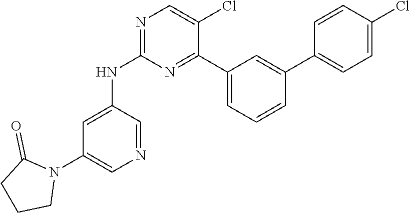 [SMILES: O=C1CCCN1c1cncc(Nc2ncc(Cl)c(-c3cccc(-c4ccc(Cl)cc4)c3)n2)c1] |
| 5 | 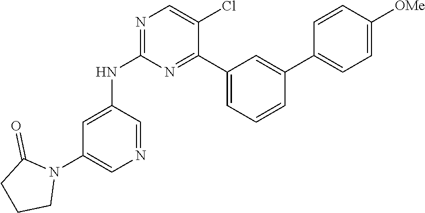 [SMILES: COc1ccc(-c2cccc(-c3nc(Nc4cncc(N5CCCC5=O)c4)ncc3Cl)c2)cc1] |
| 6 | 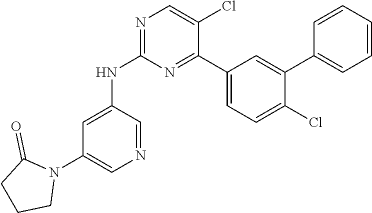 [SMILES: O=C1CCCN1c1cncc(Nc2ncc(Cl)c(-c3ccc(Cl)c(-c4ccccc4)c3)n2)c1] |
| 7 | 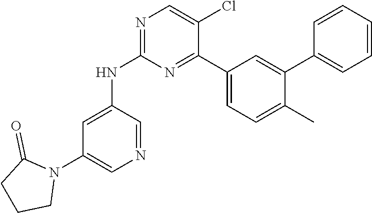 [SMILES: Cc1ccc(-c2nc(Nc3cncc(N4CCCC4=O)c3)ncc2Cl)cc1-c1ccccc1] |
| 8 | 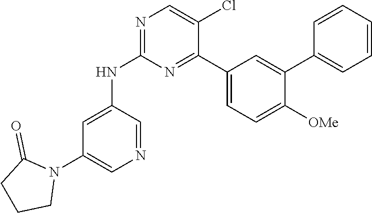 [SMILES: COc1ccc(-c2nc(Nc3cncc(N4CCCC4=O)c3)ncc2Cl)cc1-c1ccccc1] |
| 9 | 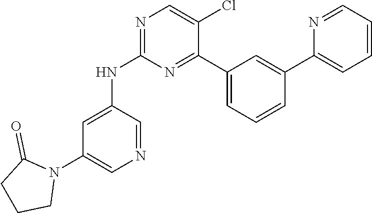 [SMILES: O=C1CCCN1c1cncc(Nc2ncc(Cl)c(-c3cccc(-c4ccccn4)c3)n2)c1] |
| 10 | 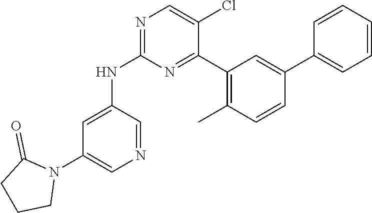 [SMILES: Cc1ccc(-c2ccccc2)cc1-c1nc(Nc2cncc(N3CCCC3=O)c2)ncc1Cl] |
| 11 | 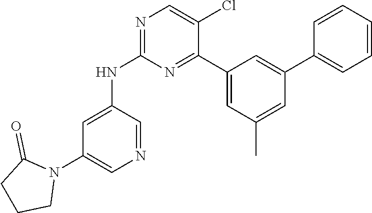 [SMILES: Cc1cc(-c2ccccc2)cc(-c2nc(Nc3cncc(N4CCCC4=O)c3)ncc2Cl)c1] |
| 12 | 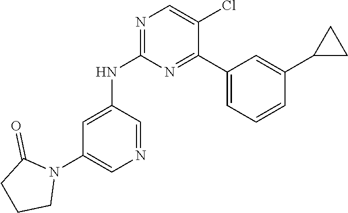 [SMILES: O=C1CCCN1c1cncc(Nc2ncc(Cl)c(-c3cccc(C4CC4)c3)n2)c1] |
| 13 | 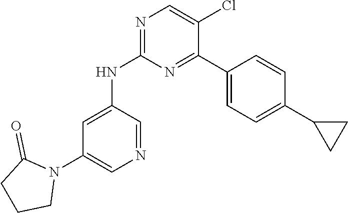 [SMILES: O=C1CCCN1c1cncc(Nc2ncc(Cl)c(-c3ccc(C4CC4)cc3)n2)c1] |
| 14 | 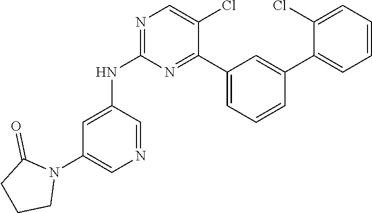 [SMILES: O=C1CCCN1c1cncc(Nc2ncc(Cl)c(-c3cccc(-c4ccccc4Cl)c3)n2)c1] |
| 15 | 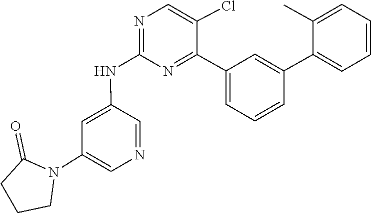 [SMILES: Cc1ccccc1-c1cccc(-c2nc(Nc3cncc(N4CCCC4=O)c3)ncc2Cl)c1] |
| 16 | 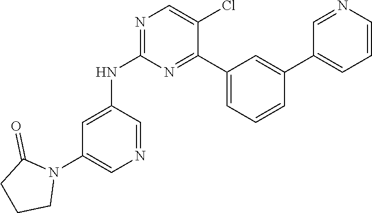 [SMILES: O=C1CCCN1c1cncc(Nc2ncc(Cl)c(-c3cccc(-c4cccnc4)c3)n2)c1] |
| 17 | 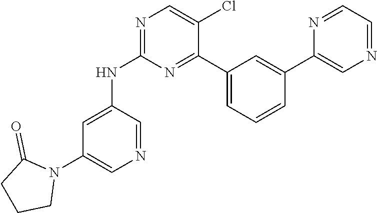 [SMILES: O=C1CCCN1c1cncc(Nc2ncc(Cl)c(-c3cccc(-c4cnccn4)c3)n2)c1] |
| 18 | 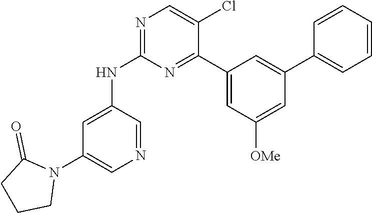 [SMILES: COc1cc(-c2ccccc2)cc(-c2nc(Nc3cncc(N4CCCC4=O)c3)ncc2Cl)c1] |
| 19 | 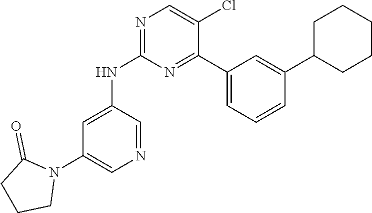 [SMILES: O=C1CCCN1c1cncc(Nc2ncc(Cl)c(-c3cccc(C4CCCCC4)c3)n2)c1] |
| 20 | 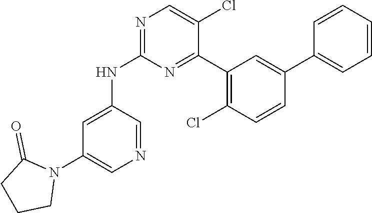 [SMILES: O=C1CCCN1c1cncc(Nc2ncc(Cl)c(-c3cc(-c4ccccc4)ccc3Cl)n2)c1] |
| 21 | 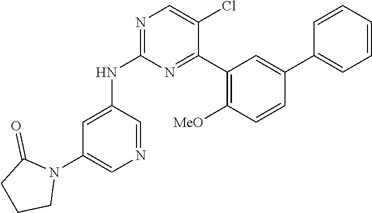 [SMILES: COc1ccc(-c2ccccc2)cc1-c1nc(Nc2cncc(N3CCCC3=O)c2)ncc1Cl] |
| 22 | 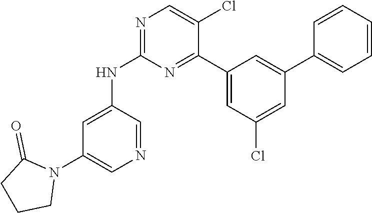 [SMILES: O=C1CCCN1c1cncc(Nc2ncc(Cl)c(-c3cc(Cl)cc(-c4ccccc4)c3)n2)c1] |
| 24 | 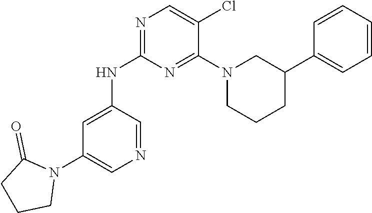 [SMILES: O=C1CCCN1c1cncc(Nc2ncc(Cl)c(N3CCCC(c4ccccc4)C3)n2)c1] |
| 26 | 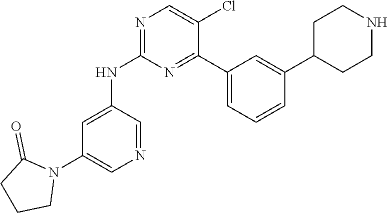 [SMILES: O=C1CCCN1c1cncc(Nc2ncc(Cl)c(-c3cccc(C4CCNCC4)c3)n2)c1] |
| 27 | 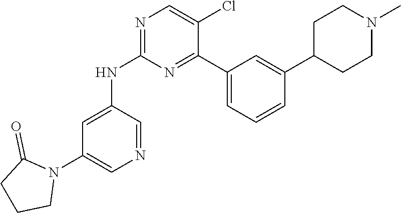 [SMILES: CN1CCC(c2cccc(-c3nc(Nc4cncc(N5CCCC5=O)c4)ncc3Cl)c2)CC1] |
| 28 | 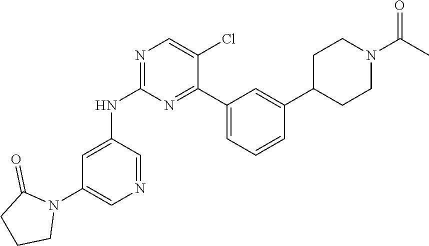 [SMILES: CC(=O)N1CCC(c2cccc(-c3nc(Nc4cncc(N5CCCC5=O)c4)ncc3Cl)c2)CC1] |
| 29, Enant A and Enant B | 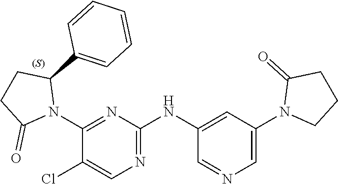 |
| | 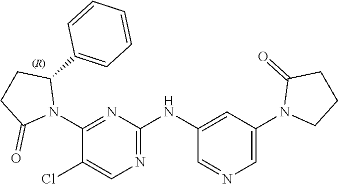 |
| 30 | 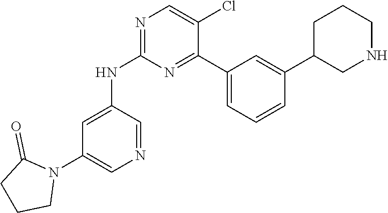 [SMILES: O=C1CCCN1c1cncc(Nc2ncc(Cl)c(-c3cccc(C4CCCNC4)c3)n2)c1] |
| 32 | 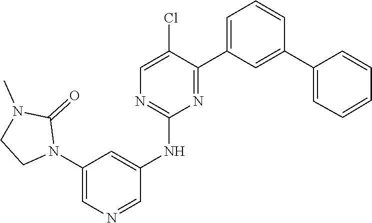 [SMILES: CN1CCN(c2cncc(Nc3ncc(Cl)c(-c4cccc(-c5ccccc5)c4)n3)c2)C1=O] |
| 33 | 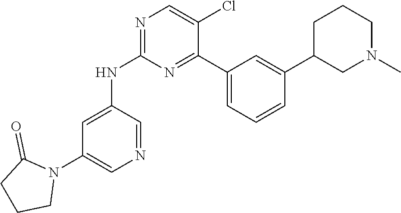 [SMILES: CN1CCCC(c2cccc(-c3nc(Nc4cncc(N5CCCC5=O)c4)ncc3Cl)c2)C1] |
| 34 | 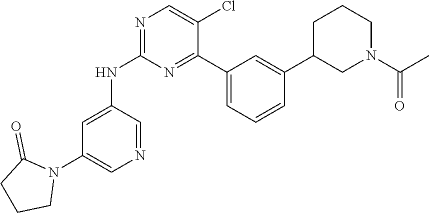 [SMILES: CC(=O)N1CCCC(c2cccc(-c3nc(Nc4cncc(N5CCCC5=O)c4)ncc3Cl)c2)C1] |
| 35 | 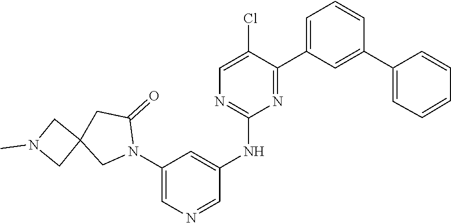 [SMILES: CN1CC2(CC(=O)N(c3cncc(Nc4ncc(Cl)c(-c5cccc(-c6ccccc6)c5)n4)c3)C2)C1] |
| 36 | 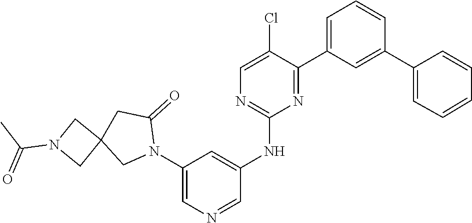 [SMILES: CC(=O)N1CC2(CC(=O)N(c3cncc(Nc4ncc(Cl)c(-c5cccc(-c6ccccc6)c5)n4)c3)C2)C1] |
| 37 | 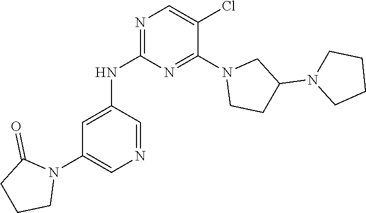 [SMILES: O=C1CCCN1c1cncc(Nc2ncc(Cl)c(N3CCC(N4CCCC4)C3)n2)c1] |
| 38 | 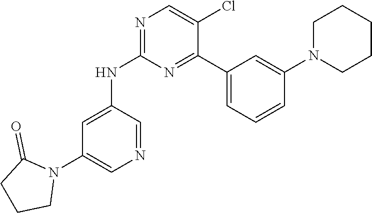 [SMILES: O=C1CCCN1c1cncc(Nc2ncc(Cl)c(-c3cccc(N4CCCCC4)c3)n2)c1] |
| 39 | 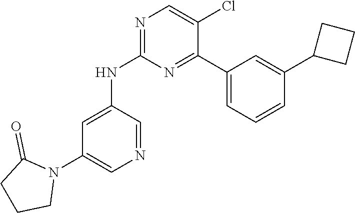 [SMILES: O=C1CCCN1c1cncc(Nc2ncc(Cl)c(-c3cccc(C4CCC4)c3)n2)c1] |
| 40 | 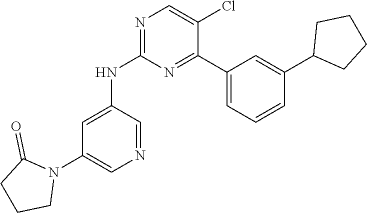 [SMILES: O=C1CCCN1c1cncc(Nc2ncc(Cl)c(-c3cccc(C4CCCC4)c3)n2)c1] |
| 41 | 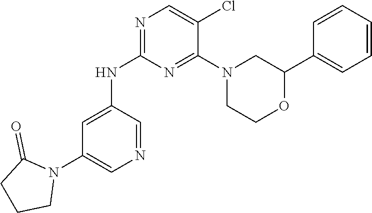 [SMILES: O=C1CCCN1c1cncc(Nc2ncc(Cl)c(N3CCOC(c4ccccc4)C3)n2)c1] |
| 42 | 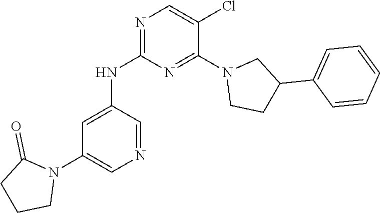 [SMILES: O=C1CCCN1c1cncc(Nc2ncc(Cl)c(N3CCC(c4ccccc4)C3)n2)c1] |
| 43 | 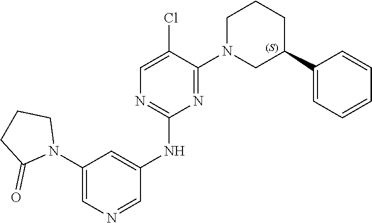 |
| 44 | 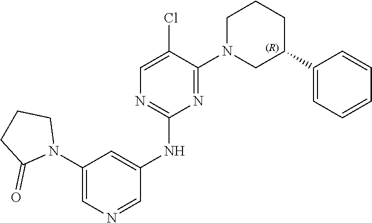 |
| 45 | 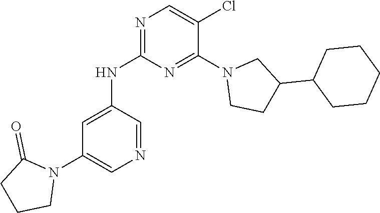 [SMILES: O=C1CCCN1c1cncc(Nc2ncc(Cl)c(N3CCC(C4CCCCC4)C3)n2)c1] |
| 46 | 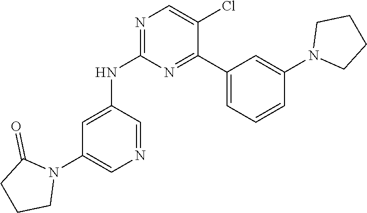 [SMILES: O=C1CCCN1c1cncc(Nc2ncc(Cl)c(-c3cccc(N4CCCC4)c3)n2)c1] |
| 47 | 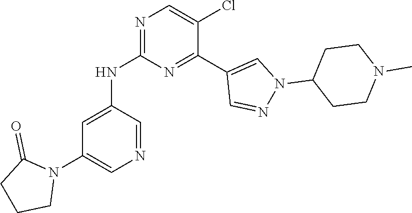 [SMILES: CN1CCC(n2cc(-c3nc(Nc4cncc(N5CCCC5=O)c4)ncc3Cl)cn2)CC1] |
| 48 | 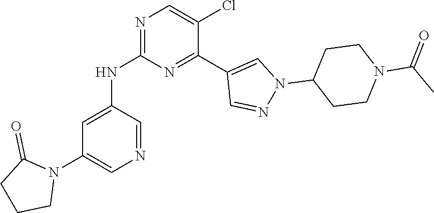 [SMILES: CC(=O)N1CCC(n2cc(-c3nc(Nc4cncc(N5CCCC5=O)c4)ncc3Cl)cn2)CC1] |
| 49 | 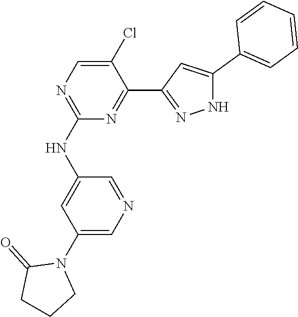 [SMILES: O=C1CCCN1c1cncc(Nc2ncc(Cl)c(-c3cc(-c4ccccc4)[nH]n3)n2)c1] |
| 50 | 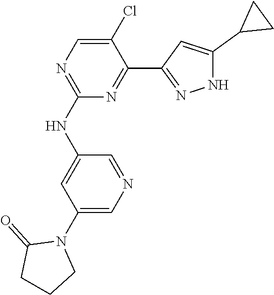 [SMILES: O=C1CCCN1c1cncc(Nc2ncc(Cl)c(-c3cc(C4CC4)[nH]n3)n2)c1] |
| 90 | 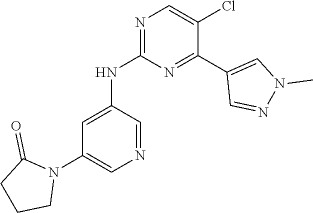 [SMILES: Cn1cc(-c2nc(Nc3cncc(N4CCCC4=O)c3)ncc2Cl)cn1] |
| 92 | 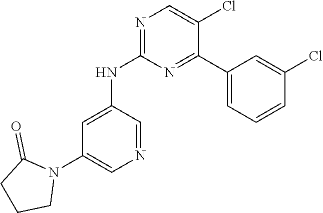 [SMILES: O=C1CCCN1c1cncc(Nc2ncc(Cl)c(-c3cccc(Cl)c3)n2)c1] |
| 93 | 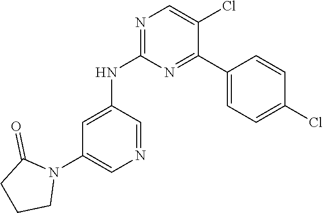 [SMILES: O=C1CCCN1c1cncc(Nc2ncc(Cl)c(-c3ccc(Cl)cc3)n2)c1] |
| 94 | 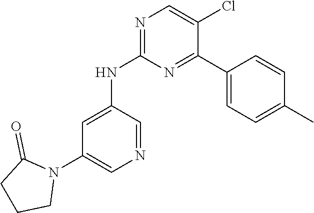 [SMILES: Cc1ccc(-c2nc(Nc3cncc(N4CCCC4=O)c3)ncc2Cl)cc1] |
| 95 | 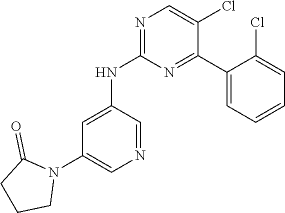 [SMILES: O=C1CCCN1c1cncc(Nc2ncc(Cl)c(-c3ccccc3Cl)n2)c1] |
| 96 | 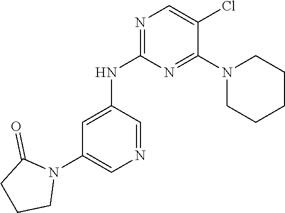 [SMILES: O=C1CCCN1c1cncc(Nc2ncc(Cl)c(N3CCCCC3)n2)c1] |
| 97 | 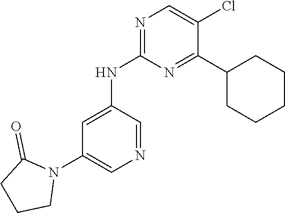 [SMILES: O=C1CCCN1c1cncc(Nc2ncc(Cl)c(C3CCCCC3)n2)c1] |
| 98 | 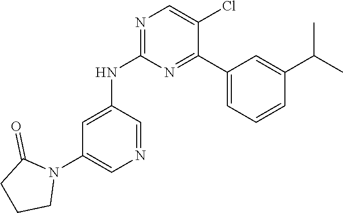 [SMILES: CC(C)c1cccc(-c2nc(Nc3cncc(N4CCCC4=O)c3)ncc2Cl)c1] |
| 99 | 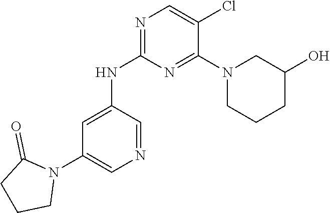 [SMILES: O=C1CCCN1c1cncc(Nc2ncc(Cl)c(N3CCCC(O)C3)n2)c1] |
| 100 | 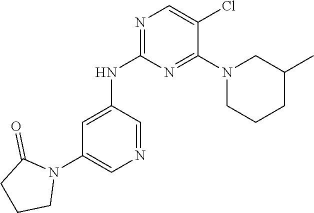 [SMILES: CC1CCCN(c2nc(Nc3cncc(N4CCCC4=O)c3)ncc2Cl)C1] |
| 101 | 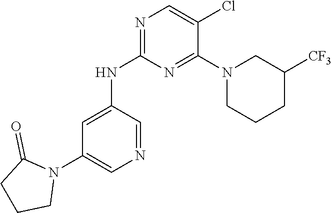 [SMILES: O=C1CCCN1c1cncc(Nc2ncc(Cl)c(N3CCCC(C(F)(F)F)C3)n2)c1] |
| 102 | 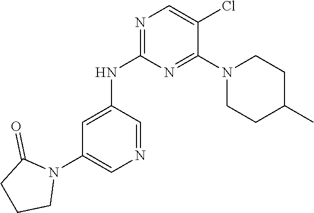 [SMILES: CC1CCN(c2nc(Nc3cncc(N4CCCC4=O)c3)ncc2Cl)CC1] |
| 103 | 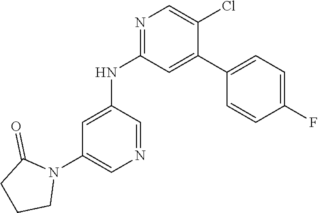 [SMILES: O=C1CCCN1c1cncc(Nc2cc(-c3ccc(F)cc3)c(Cl)cn2)c1] |
| 104 | 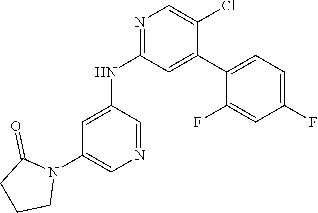 [SMILES: O=C1CCCN1c1cncc(Nc2cc(-c3ccc(F)cc3F)c(Cl)cn2)c1] |
| 105 | 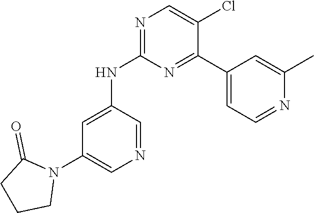 [SMILES: Cc1cc(-c2nc(Nc3cncc(N4CCCC4=O)c3)ncc2Cl)ccn1] |
| 106 | 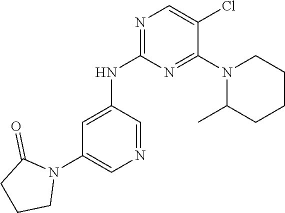 [SMILES: CC1CCCCN1c1nc(Nc2cncc(N3CCCC3=O)c2)ncc1Cl] |
| 107 | 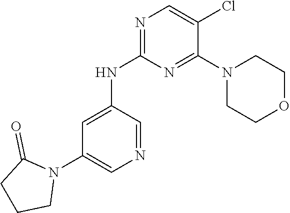 [SMILES: O=C1CCCN1c1cncc(Nc2ncc(Cl)c(N3CCOCC3)n2)c1] |
| 108 | 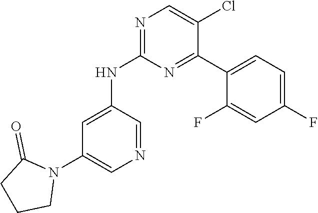 [SMILES: O=C1CCCN1c1cncc(Nc2ncc(Cl)c(-c3ccc(F)cc3F)n2)c1] |
| 109 | 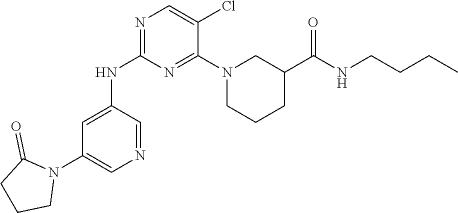 [SMILES: CCCCNC(=O)C1CCCN(c2nc(Nc3cncc(N4CCCC4=O)c3)ncc2Cl)C1] |
| 110 | 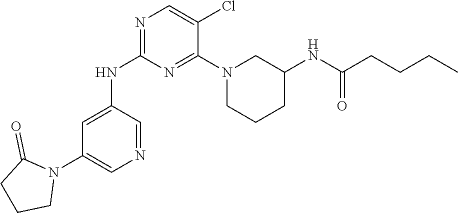 [SMILES: CCCCC(=O)NC1CCCN(c2nc(Nc3cncc(N4CCCC4=O)c3)ncc2Cl)C1] |
| 111 | 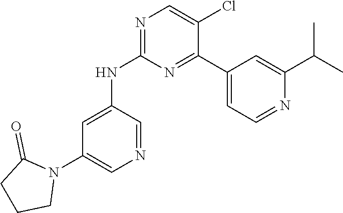 [SMILES: CC(C)c1cc(-c2nc(Nc3cncc(N4CCCC4=O)c3)ncc2Cl)ccn1] |
| 112 | 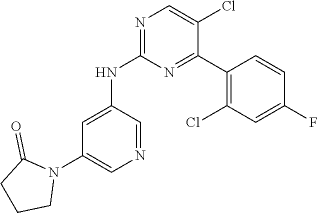 [SMILES: O=C1CCCN1c1cncc(Nc2ncc(Cl)c(-c3ccc(F)cc3Cl)n2)c1] |
| 113 | 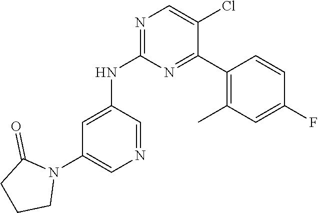 [SMILES: Cc1cc(F)ccc1-c1nc(Nc2cncc(N3CCCC3=O)c2)ncc1Cl] |
| 114 | 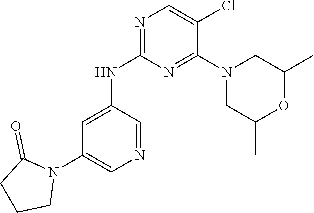 [SMILES: CC1CN(c2nc(Nc3cncc(N4CCCC4=O)c3)ncc2Cl)CC(C)O1] |
| 115 | 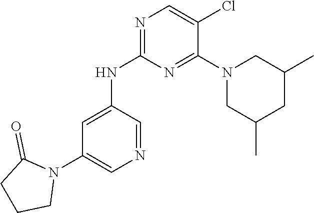 [SMILES: CC1CC(C)CN(c2nc(Nc3cncc(N4CCCC4=O)c3)ncc2Cl)C1] |
| 116 | 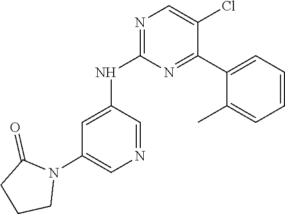 [SMILES: Cc1ccccc1-c1nc(Nc2cncc(N3CCCC3=O)c2)ncc1Cl] |
| 117 | 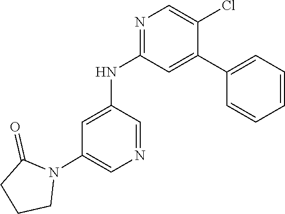 [SMILES: O=C1CCCN1c1cncc(Nc2cc(-c3ccccc3)c(Cl)cn2)c1] |
| 118 | 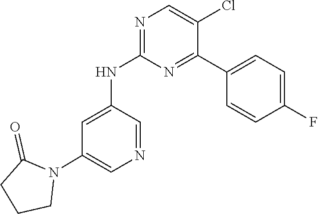 [SMILES: O=C1CCCN1c1cncc(Nc2ncc(Cl)c(-c3ccc(F)cc3)n2)c1] |
| 119 | 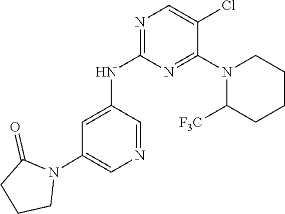 [SMILES: O=C1CCCN1c1cncc(Nc2ncc(Cl)c(N3CCCCC3C(F)(F)F)n2)c1] |
| 120 | 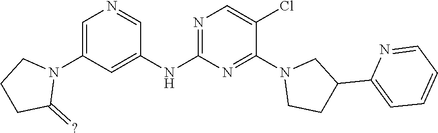 |
| 121 | 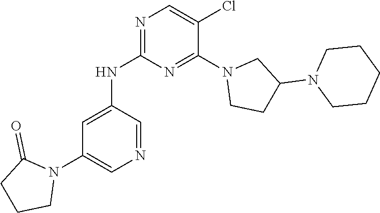 [SMILES: O=C1CCCN1c1cncc(Nc2ncc(Cl)c(N3CCC(N4CCCCC4)C3)n2)c1] |
| 122 | 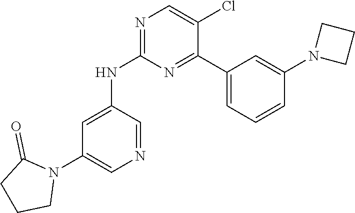 [SMILES: O=C1CCCN1c1cncc(Nc2ncc(Cl)c(-c3cccc(N4CCC4)c3)n2)c1] |
| 123 | 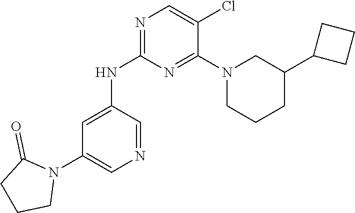 [SMILES: O=C1CCCN1c1cncc(Nc2ncc(Cl)c(N3CCCC(C4CCC4)C3)n2)c1] |
| 124 | 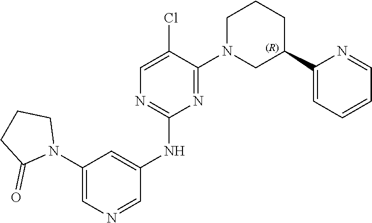 |
| 125 | 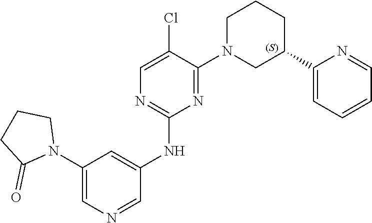 |
| 126 | 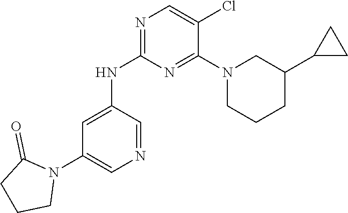 [SMILES: O=C1CCCN1c1cncc(Nc2ncc(Cl)c(N3CCCC(C4CC4)C3)n2)c1] |
| 127 | 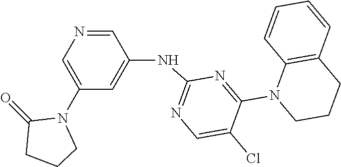 [SMILES: O=C1CCCN1c1cncc(Nc2ncc(Cl)c(N3CCCc4ccccc43)n2)c1] |
| 128 | 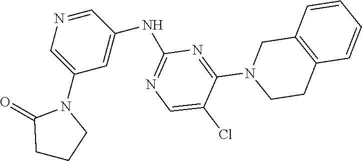 [SMILES: O=C1CCCN1c1cncc(Nc2ncc(Cl)c(N3CCc4ccccc4C3)n2)c1] |
| 129 | 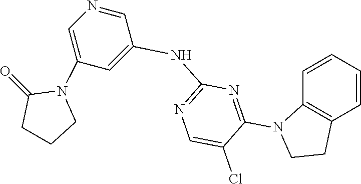 [SMILES: O=C1CCCN1c1cncc(Nc2ncc(Cl)c(N3CCc4ccccc43)n2)c1] |
| 130 | 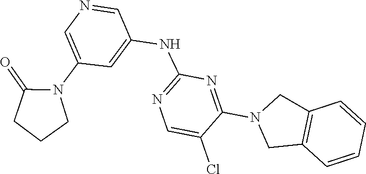 [SMILES: O=C1CCCN1c1cncc(Nc2ncc(Cl)c(N3Cc4ccccc4C3)n2)c1] |
| 131 | 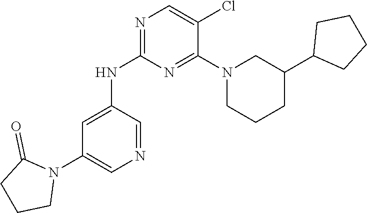 [SMILES: O=C1CCCN1c1cncc(Nc2ncc(Cl)c(N3CCCC(C4CCCC4)C3)n2)c1] |
| 132 | 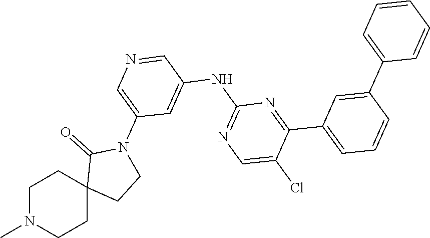 [SMILES: CN1CCC2(CC1)CCN(c1cncc(Nc3ncc(Cl)c(-c4cccc(-c5ccccc5)c4)n3)c1)C2=O] |
| 133 | 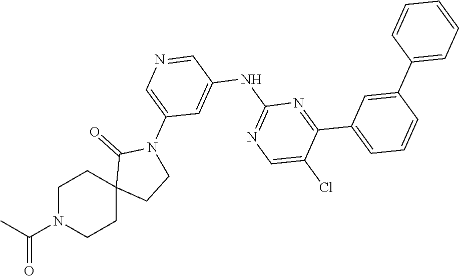 [SMILES: CC(=O)N1CCC2(CC1)CCN(c1cncc(Nc3ncc(Cl)c(-c4cccc(-c5ccccc5)c4)n3)c1)C2=O] |
| 134 | 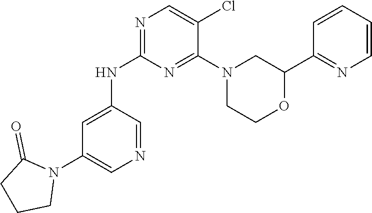 [SMILES: O=C1CCCN1c1cncc(Nc2ncc(Cl)c(N3CCOC(c4ccccn4)C3)n2)c1] |
| 135 | 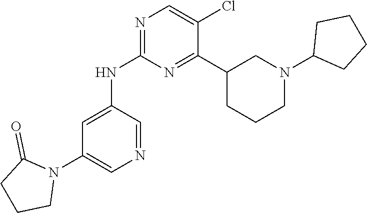 [SMILES: O=C1CCCN1c1cncc(Nc2ncc(Cl)c(C3CCCN(C4CCCC4)C3)n2)c1] |
| 136 | 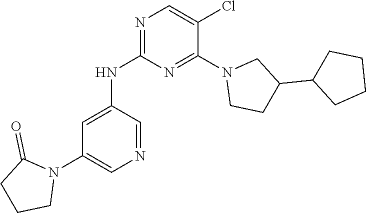 [SMILES: O=C1CCCN1c1cncc(Nc2ncc(Cl)c(N3CCC(C4CCCC4)C3)n2)c1] |
| 137 | 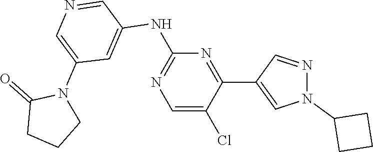 [SMILES: O=C1CCCN1c1cncc(Nc2ncc(Cl)c(-c3cnn(C4CCC4)c3)n2)c1] |
| 138 | 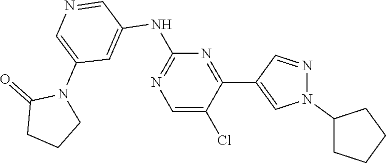 [SMILES: O=C1CCCN1c1cncc(Nc2ncc(Cl)c(-c3cnn(C4CCCC4)c3)n2)c1] |
| 139 | 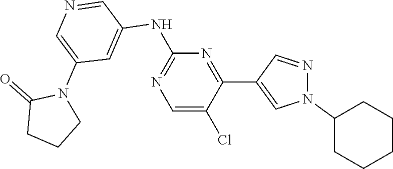 [SMILES: O=C1CCCN1c1cncc(Nc2ncc(Cl)c(-c3cnn(C4CCCCC4)c3)n2)c1] |
| 140 | 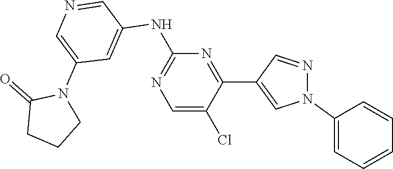 [SMILES: O=C1CCCN1c1cncc(Nc2ncc(Cl)c(-c3cnn(-c4ccccc4)c3)n2)c1] |
| 141 | 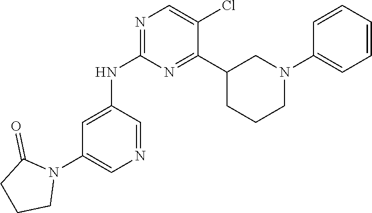 [SMILES: O=C1CCCN1c1cncc(Nc2ncc(Cl)c(C3CCCN(c4ccccc4)C3)n2)c1] |
| 142 | 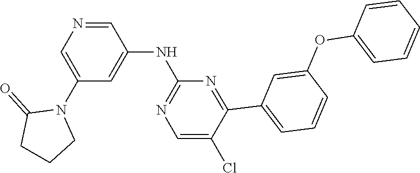 |
| 143 | 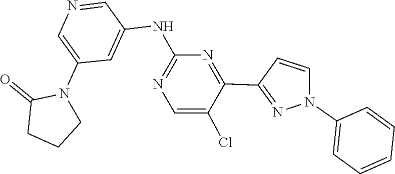 [SMILES: O=C1CCCN1c1cncc(Nc2ncc(Cl)c(-c3ccn(-c4ccccc4)n3)n2)c1] |
| 144 | 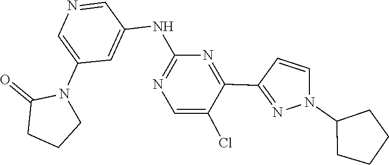 [SMILES: O=C1CCCN1c1cncc(Nc2ncc(Cl)c(-c3ccn(C4CCCC4)n3)n2)c1] |
| 145 | 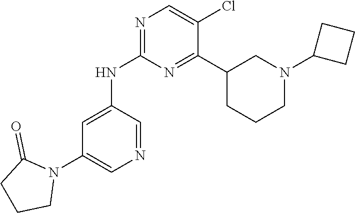 [SMILES: O=C1CCCN1c1cncc(Nc2ncc(Cl)c(C3CCCN(C4CCC4)C3)n2)c1] |
| 146 | 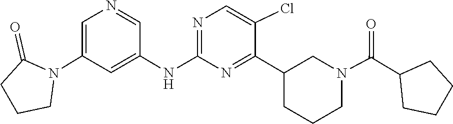 [SMILES: O=C(C1CCCC1)N1CCCC(c2nc(Nc3cncc(N4CCCC4=O)c3)ncc2Cl)C1] |
| 147 | 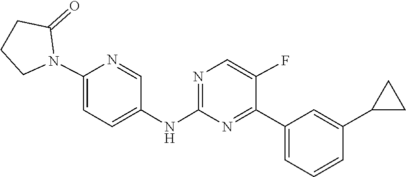 [SMILES: O=C1CCCN1c1ccc(Nc2ncc(F)c(-c3cccc(C4CC4)c3)n2)cn1] |
| 148 | 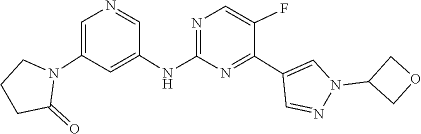 [SMILES: O=C1CCCN1c1cncc(Nc2ncc(F)c(-c3cnn(C4COC4)c3)n2)c1] |
| 149 | 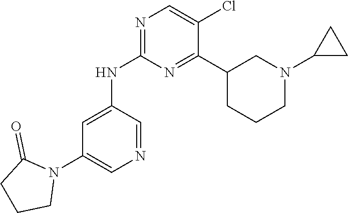 [SMILES: O=C1CCCN1c1cncc(Nc2ncc(Cl)c(C3CCCN(C4CC4)C3)n2)c1] |
| 150 | 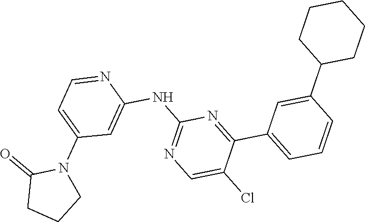 [SMILES: O=C1CCCN1c1ccnc(Nc2ncc(Cl)c(-c3cccc(C4CCCCC4)c3)n2)c1] |
| 151 | 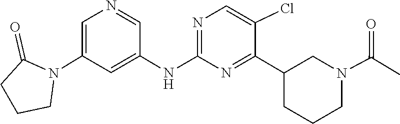 [SMILES: CC(=O)N1CCCC(c2nc(Nc3cncc(N4CCCC4=O)c3)ncc2Cl)C1] |
| 152 | 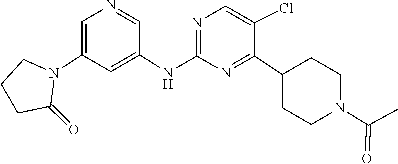 [SMILES: CC(=O)N1CCC(c2nc(Nc3cncc(N4CCCC4=O)c3)ncc2Cl)CC1] |
| 153 | 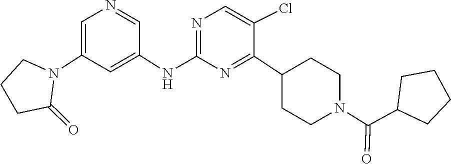 [SMILES: O=C(C1CCCC1)N1CCC(c2nc(Nc3cncc(N4CCCC4=O)c3)ncc2Cl)CC1] |
| 154 | 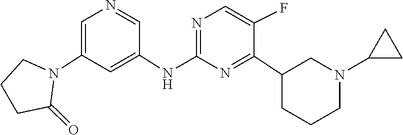 [SMILES: O=C1CCCN1c1cncc(Nc2ncc(F)c(C3CCCN(C4CC4)C3)n2)c1] |
| 155 | 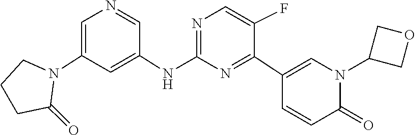 [SMILES: O=C1CCCN1c1cncc(Nc2ncc(F)c(-c3ccc(=O)n(C4COC4)c3)n2)c1] |
| 156 | 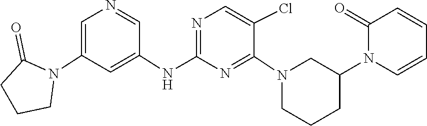 [SMILES: O=C1CCCN1c1cncc(Nc2ncc(Cl)c(N3CCCC(n4ccccc4=O)C3)n2)c1] |
| 157 | 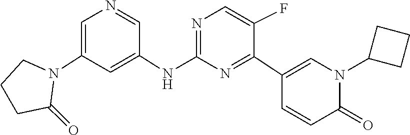 [SMILES: O=C1CCCN1c1cncc(Nc2ncc(F)c(-c3ccc(=O)n(C4CCC4)c3)n2)c1] |
| 158 | 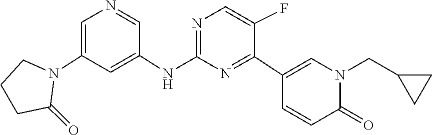 [SMILES: O=C1CCCN1c1cncc(Nc2ncc(F)c(-c3ccc(=O)n(CC4CC4)c3)n2)c1] |
| 159 | 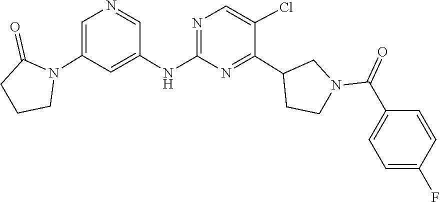 [SMILES: O=C(c1ccc(F)cc1)N1CCC(c2nc(Nc3cncc(N4CCCC4=O)c3)ncc2Cl)C1] |
| 160 | 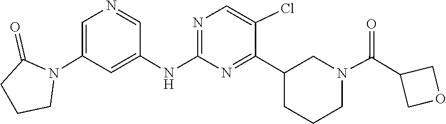 [SMILES: O=C(C1COC1)N1CCCC(c2nc(Nc3cncc(N4CCCC4=O)c3)ncc2Cl)C1] |
| 161 | 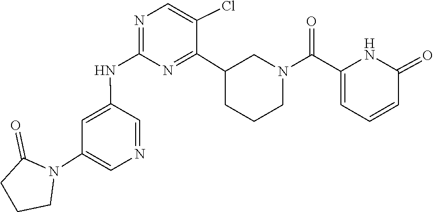 [SMILES: O=C(c1cccc(=O)[nH]1)N1CCCC(c2nc(Nc3cncc(N4CCCC4=O)c3)ncc2Cl)C1] |
| 162 | 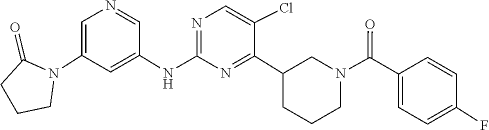 [SMILES: O=C(c1ccc(F)cc1)N1CCCC(c2nc(Nc3cncc(N4CCCC4=O)c3)ncc2Cl)C1] |
| 163 | 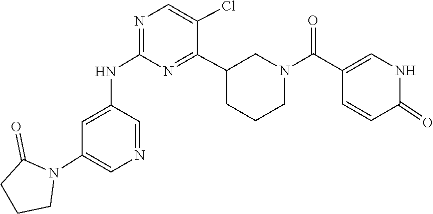 [SMILES: O=C(c1ccc(=O)[nH]c1)N1CCCC(c2nc(Nc3cncc(N4CCCC4=O)c3)ncc2Cl)C1] |
| 164 | 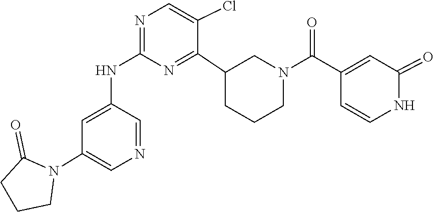 [SMILES: O=C(c1cc[nH]c(=O)c1)N1CCCC(c2nc(Nc3cncc(N4CCCC4=O)c3)ncc2Cl)C1] |
| 165 | 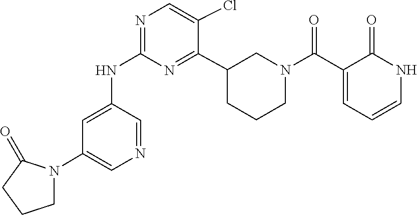 [SMILES: O=C(c1ccc[nH]c1=O)N1CCCC(c2nc(Nc3cncc(N4CCCC4=O)c3)ncc2Cl)C1] |

TABLE 2
Representative Compounds of Formula (I′), (I), and (III)

| Cmpd No. | Structure |
|---|---|
| 51 | 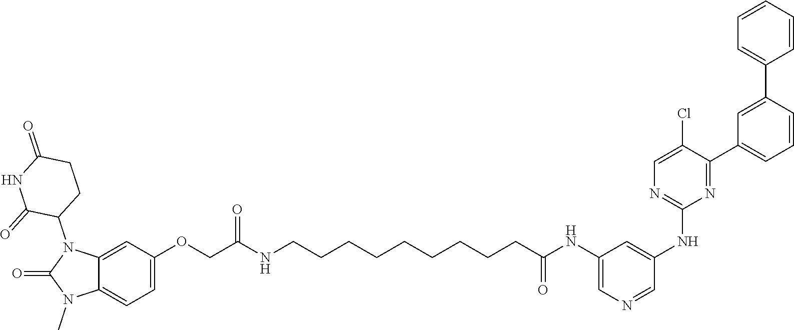 [SMILES: Cn1c(=O)n(C2CCC(=O)NC2=O)c2cc(OCC(=O)NCCCCCCCCCC(=O)Nc3cncc(Nc4ncc(Cl)c(-c5cccc(-c6ccccc6)c5)n4)c3)ccc21] |
| 52 | 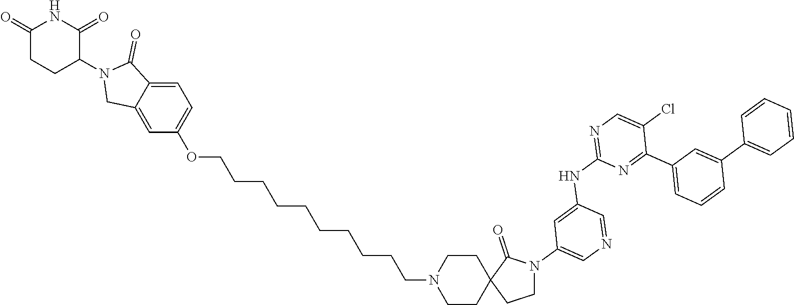 [SMILES: O=C1CCC(N2Cc3cc(OCCCCCCCCCCN4CCC5(CC4)CCN(c4cncc(Nc6ncc(Cl)c(-c7cccc(-c8ccccc8)c7)n6)c4)C5=O)ccc3C2=O)C(=O)N1] |
| 53 | 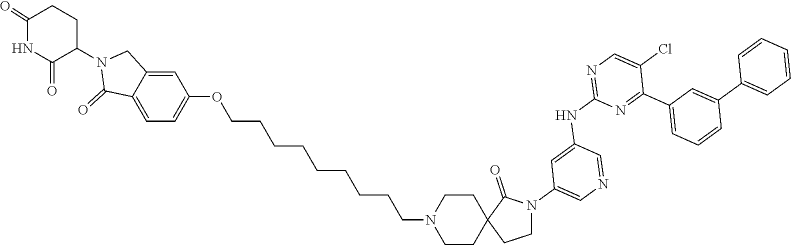 [SMILES: O=C1CCC(N2Cc3cc(OCCCCCCCCCN4CCC5(CC4)CCN(c4cncc(Nc6ncc(Cl)c(-c7cccc(-c8ccccc8)c7)n6)c4)C5=O)ccc3C2=O)C(=O)N1] |
| 54 | 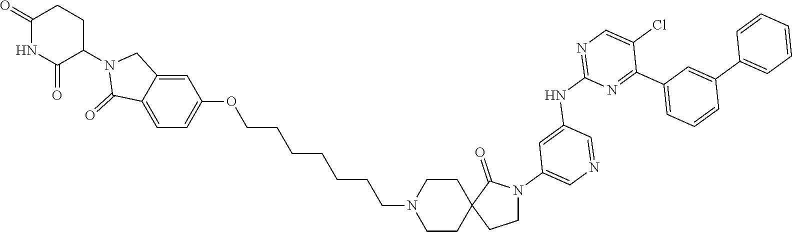 [SMILES: O=C1CCC(N2Cc3cc(OCCCCCCCN4CCC5(CC4)CCN(c4cncc(Nc6ncc(Cl)c(-c7cccc(-c8ccccc8)c7)n6)c4)C5=O)ccc3C2=O)C(=O)N1] |
| 55 | 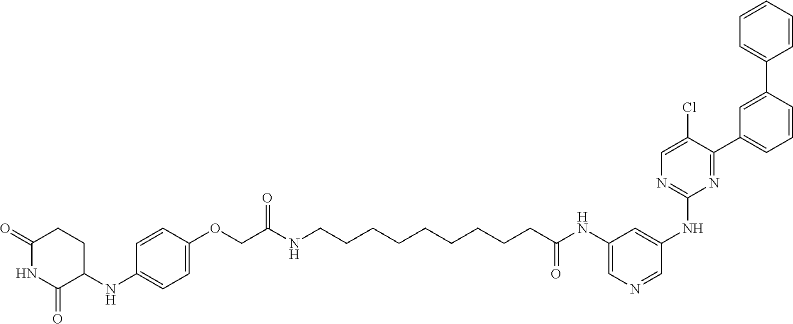 [SMILES: O=C(COc1ccc(NC2CCC(=O)NC2=O)cc1)NCCCCCCCCCC(=O)Nc1cncc(Nc2ncc(Cl)c(-c3cccc(-c4ccccc4)c3)n2)c1] |
| 56 | 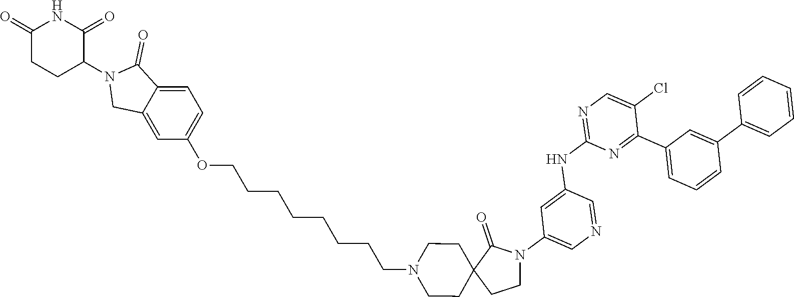 [SMILES: O=C1CCC(N2Cc3cc(OCCCCCCCCN4CCC5(CC4)CCN(c4cncc(Nc6ncc(Cl)c(-c7cccc(-c8ccccc8)c7)n6)c4)C5=O)ccc3C2=O)C(=O)N1] |
| 58 | 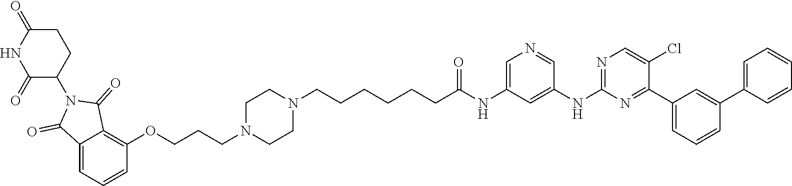 [SMILES: O=C1CCC(N2C(=O)c3cccc(OCCCN4CCN(CCCCCCC(=O)Nc5cncc(Nc6ncc(Cl)c(-c7cccc(-c8ccccc8)c7)n6)c5)CC4)c3C2=O)C(=O)N1] |
| 59 | 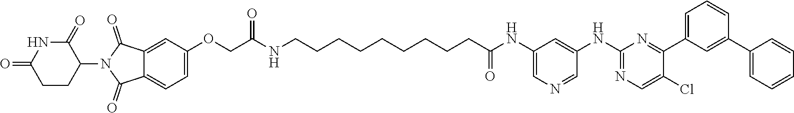 [SMILES: O=C(COc1ccc2c(c1)C(=O)N(C1CCC(=O)NC1=O)C2=O)NCCCCCCCCCC(=O)Nc1cncc(Nc2ncc(Cl)c(-c3cccc(-c4ccccc4)c3)n2)c1] |
| 60 | 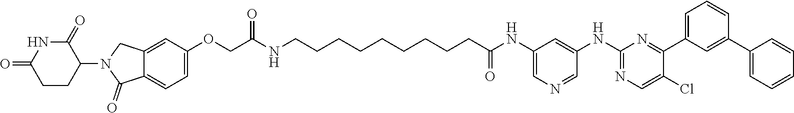 [SMILES: O=C(COc1ccc2c(c1)CN(C1CCC(=O)NC1=O)C2=O)NCCCCCCCCCC(=O)Nc1cncc(Nc2ncc(Cl)c(-c3cccc(-c4ccccc4)c3)n2)c1] |
| 61 | 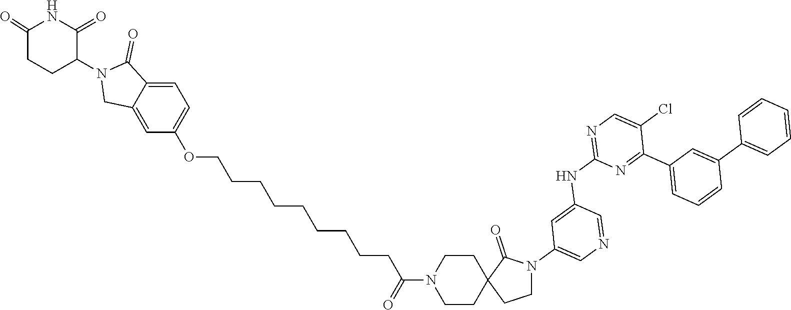 [SMILES: O=C1CCC(N2Cc3cc(OCCCCCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 62 | 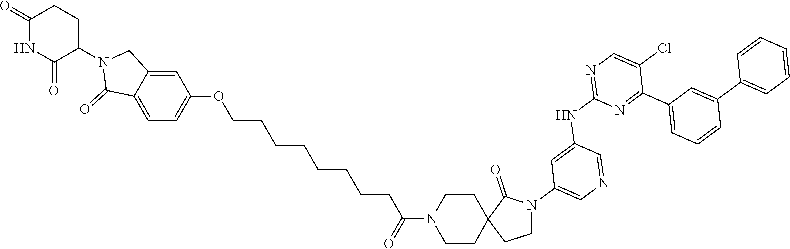 [SMILES: O=C1CCC(N2Cc3cc(OCCCCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 63 | 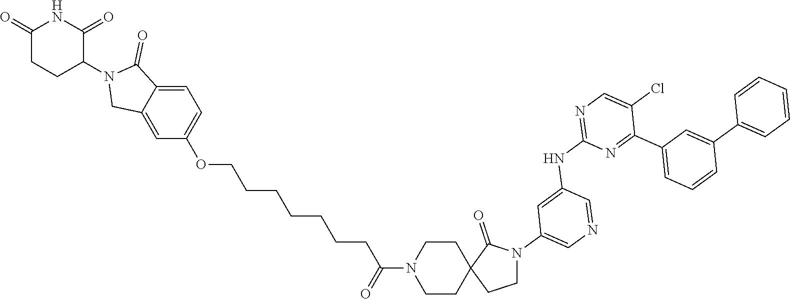 [SMILES: O=C1CCC(N2Cc3cc(OCCCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 64 | 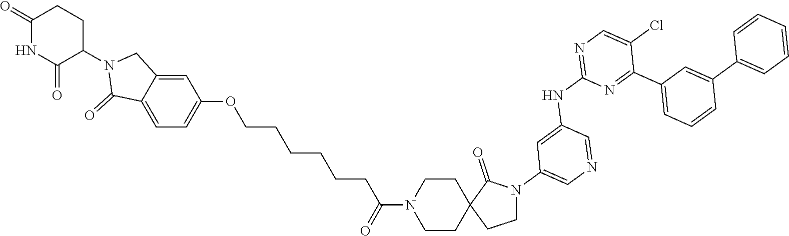 [SMILES: O=C1CCC(N2Cc3cc(OCCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 65 | 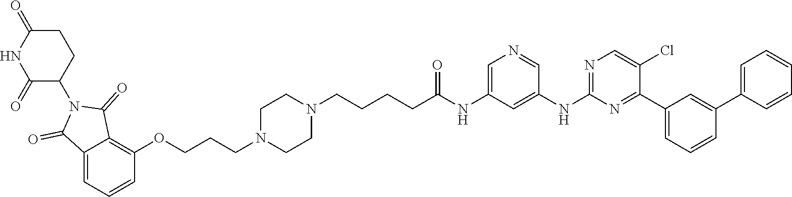 [SMILES: O=C1CCC(N2C(=O)c3cccc(OCCCN4CCN(CCCCC(=O)Nc5cncc(Nc6ncc(Cl)c(-c7cccc(-c8ccccc8)c7)n6)c5)CC4)c3C2=O)C(=O)N1] |
| 66 | 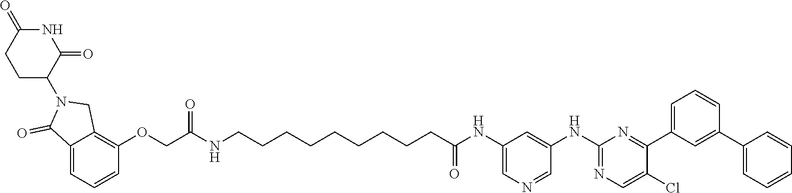 [SMILES: O=C(COc1cccc2c1CN(C1CCC(=O)NC1=O)C2=O)NCCCCCCCCCC(=O)Nc1cncc(Nc2ncc(Cl)c(-c3cccc(-c4ccccc4)c3)n2)c1] |
| 67 | 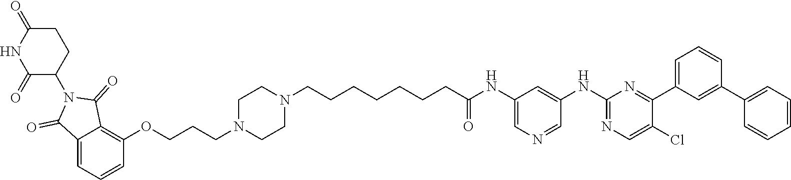 [SMILES: O=C1CCC(N2C(=O)c3cccc(OCCCN4CCN(CCCCCCCC(=O)Nc5cncc(Nc6ncc(Cl)c(-c7cccc(-c8ccccc8)c7)n6)c5)CC4)c3C2=O)C(=O)N1] |
| 68 | 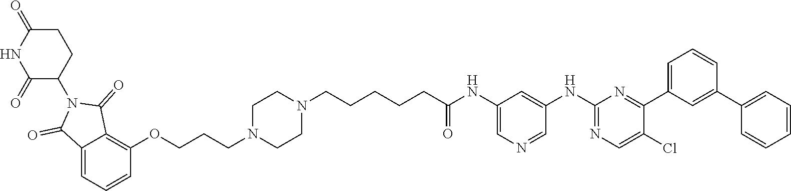 [SMILES: O=C1CCC(N2C(=O)c3cccc(OCCCN4CCN(CCCCCC(=O)Nc5cncc(Nc6ncc(Cl)c(-c7cccc(-c8ccccc8)c7)n6)c5)CC4)c3C2=O)C(=O)N1] |
| 69 | 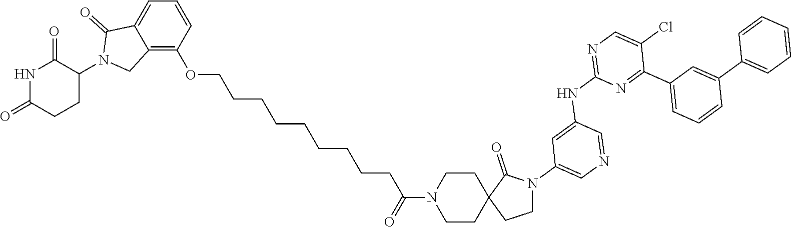 [SMILES: O=C1CCC(N2Cc3c(OCCCCCCCCCC(=O)N4CCC5(CC4)CCN(c4cncc(Nc6ncc(Cl)c(-c7cccc(-c8ccccc8)c7)n6)c4)C5=O)cccc3C2=O)C(=O)N1] |
| 70 | 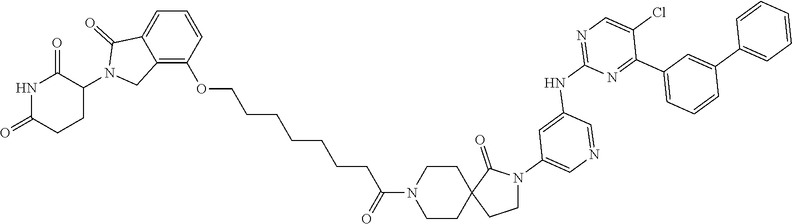 [SMILES: O=C1CCC(N2Cc3c(OCCCCCCCC(=O)N4CCC5(CC4)CCN(c4cncc(Nc6ncc(Cl)c(-c7cccc(-c8ccccc8)c7)n6)c4)C5=O)cccc3C2=O)C(=O)N1] |
| 71 | 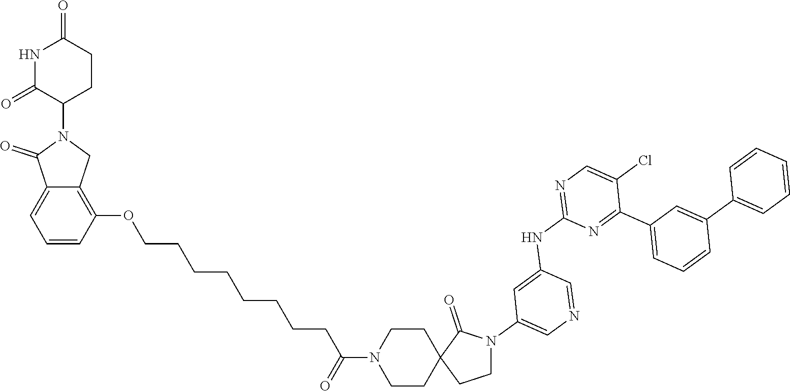 [SMILES: O=C1CCC(N2Cc3c(OCCCCCCCCC(=O)N4CCC5(CC4)CCN(c4cncc(Nc6ncc(Cl)c(-c7cccc(-c8ccccc8)c7)n6)c4)C5=O)cccc3C2=O)C(=O)N1] |
| 72 | 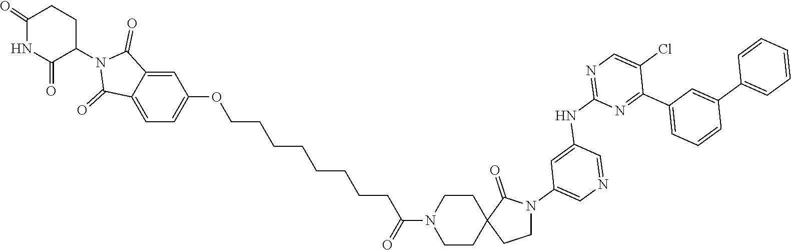 [SMILES: O=C1CCC(N2C(=O)c3ccc(OCCCCCCCCC(=O)N4CCC5(CC4)CCN(c4cncc(Nc6ncc(Cl)c(-c7cccc(-c8ccccc8)c7)n6)c4)C5=O)cc3C2=O)C(=O)N1] |
| 73 | 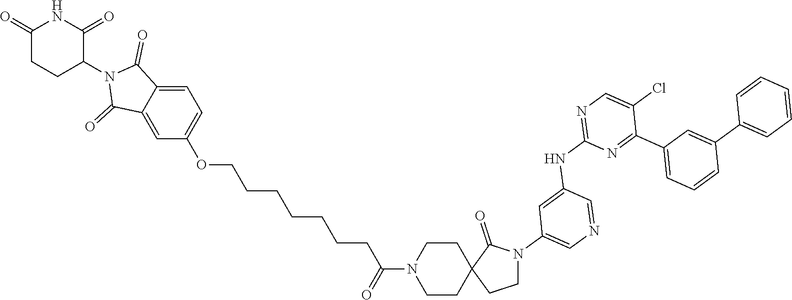 [SMILES: O=C1CCC(N2C(=O)c3ccc(OCCCCCCCC(=O)N4CCC5(CC4)CCN(c4cncc(Nc6ncc(Cl)c(-c7cccc(-c8ccccc8)c7)n6)c4)C5=O)cc3C2=O)C(=O)N1] |
| 74 | 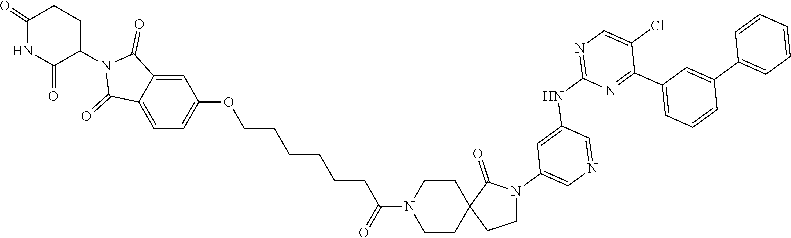 [SMILES: O=C1CCC(N2C(=O)c3ccc(OCCCCCCC(=O)N4CCC5(CC4)CCN(c4cncc(Nc6ncc(Cl)c(-c7cccc(-c8ccccc8)c7)n6)c4)C5=O)cc3C2=O)C(=O)N1] |
| 75 | 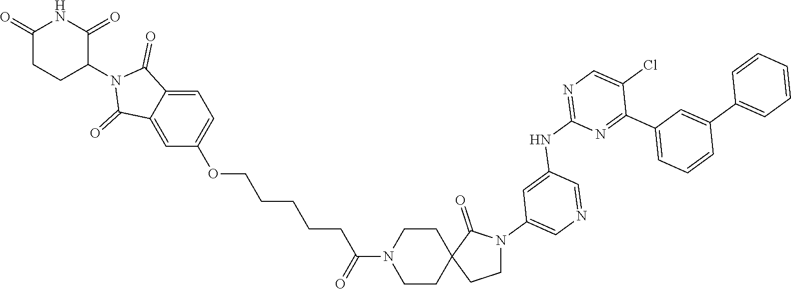 [SMILES: O=C1CCC(N2C(=O)c3ccc(OCCCCCC(=O)N4CCC5(CC4)CCN(c4cncc(Nc6ncc(Cl)c(-c7cccc(-c8ccccc8)c7)n6)c4)C5=O)cc3C2=O)C(=O)N1] |
| 76 | 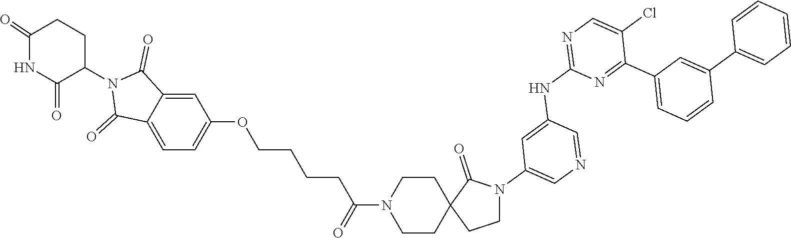 [SMILES: O=C1CCC(N2C(=O)c3ccc(OCCCCC(=O)N4CCC5(CC4)CCN(c4cncc(Nc6ncc(Cl)c(-c7cccc(-c8ccccc8)c7)n6)c4)C5=O)cc3C2=O)C(=O)N1] |
| 77 | 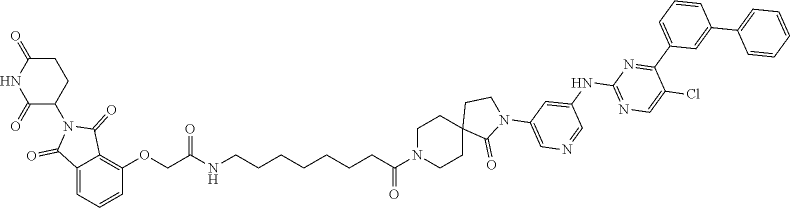 [SMILES: O=C(COc1cccc2c1C(=O)N(C1CCC(=O)NC1=O)C2=O)NCCCCCCCC(=O)N1CCC2(CC1)CCN(c1cncc(Nc3ncc(Cl)c(-c4cccc(-c5ccccc5)c4)n3)c1)C2=O] |
| 78 | 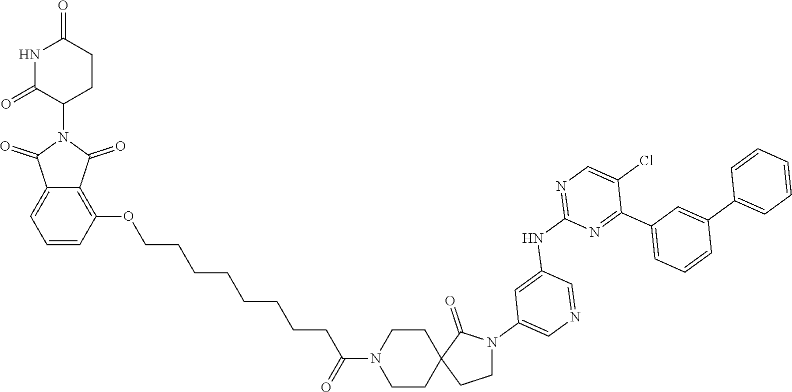 [SMILES: O=C1CCC(N2C(=O)c3cccc(OCCCCCCCCC(=O)N4CCC5(CC4)CCN(c4cncc(Nc6ncc(Cl)c(-c7cccc(-c8ccccc8)c7)n6)c4)C5=O)c3C2=O)C(=O)N1] |
| 79 | 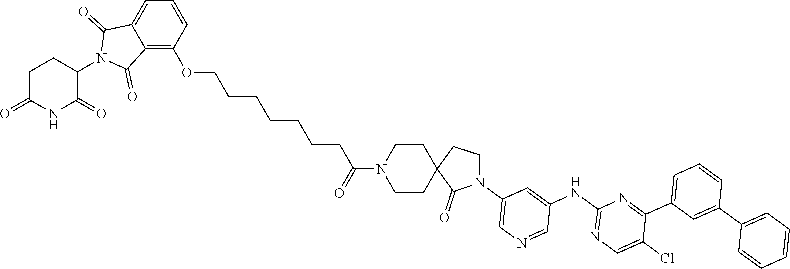 [SMILES: O=C1CCC(N2C(=O)c3cccc(OCCCCCCCC(=O)N4CCC5(CC4)CCN(c4cncc(Nc6ncc(Cl)c(-c7cccc(-c8ccccc8)c7)n6)c4)C5=O)c3C2=O)C(=O)N1] |
| 80 | 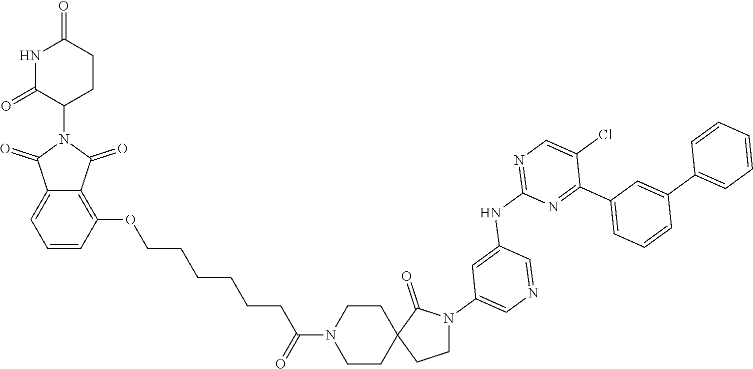 [SMILES: O=C1CCC(N2C(=O)c3cccc(OCCCCCCC(=O)N4CCC5(CC4)CCN(c4cncc(Nc6ncc(Cl)c(-c7cccc(-c8ccccc8)c7)n6)c4)C5=O)c3C2=O)C(=O)N1] |
| 81 | 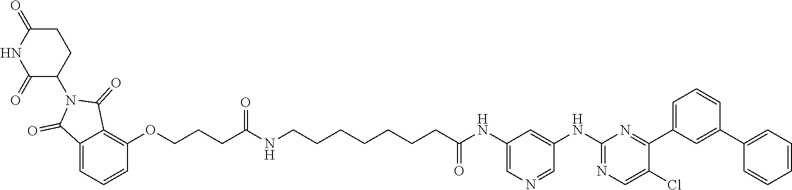 [SMILES: O=C(CCCOc1cccc2c1C(=O)N(C1CCC(=O)NC1=O)C2=O)NCCCCCCCC(=O)Nc1cncc(Nc2ncc(Cl)c(-c3cccc(-c4ccccc4)c3)n2)c1] |
| 82 | 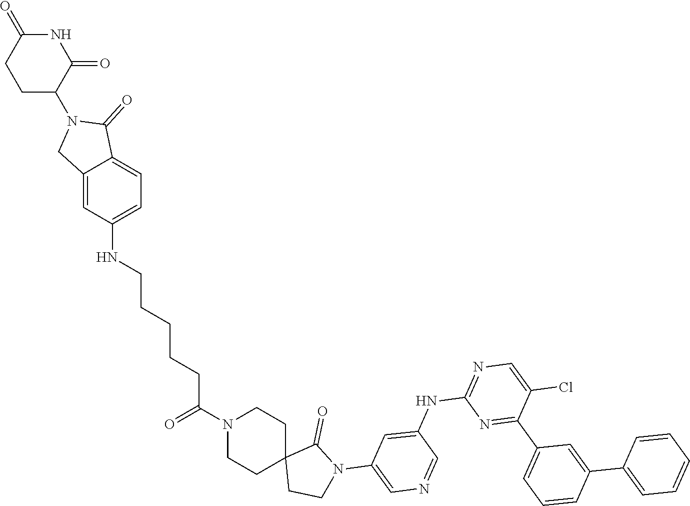 [SMILES: O=C1CCC(N2Cc3cc(NCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 83 | 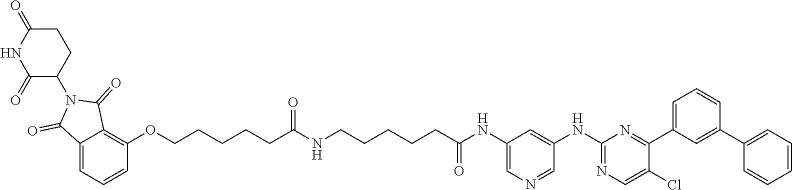 [SMILES: O=C(CCCCCOc1cccc2c1C(=O)N(C1CCC(=O)NC1=O)C2=O)NCCCCCC(=O)Nc1cncc(Nc2ncc(Cl)c(-c3cccc(-c4ccccc4)c3)n2)c1] |
| 84 | 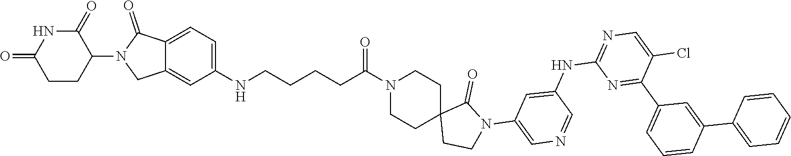 [SMILES: O=C1CCC(N2Cc3cc(NCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 85 | 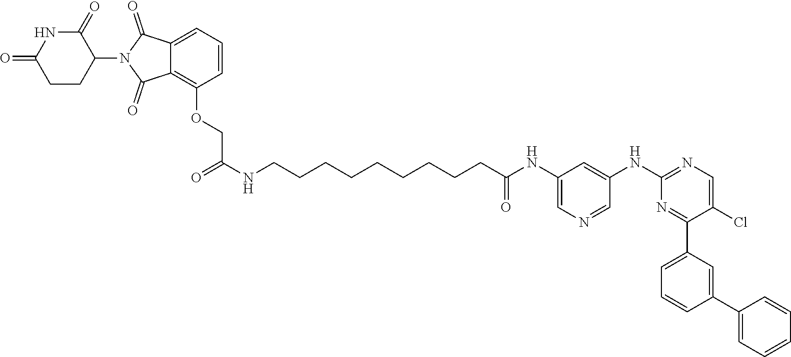 [SMILES: O=C(COc1cccc2c1C(=O)N(C1CCC(=O)NC1=O)C2=O)NCCCCCCCCCC(=O)Nc1cncc(Nc2ncc(Cl)c(-c3cccc(-c4ccccc4)c3)n2)c1] |
| 86 | 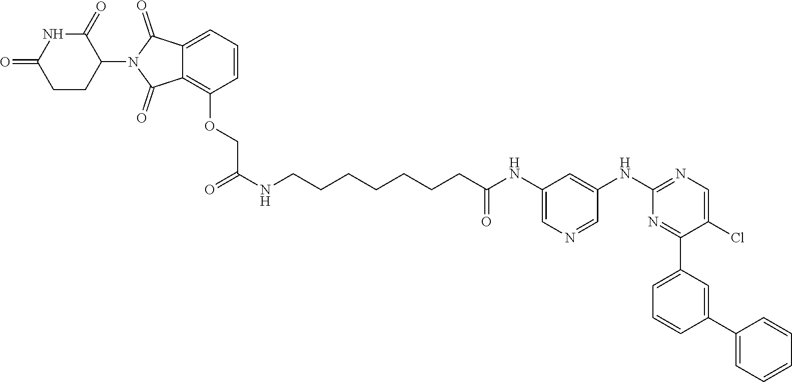 [SMILES: O=C(COc1cccc2c1C(=O)N(C1CCC(=O)NC1=O)C2=O)NCCCCCCCC(=O)Nc1cncc(Nc2ncc(Cl)c(-c3cccc(-c4ccccc4)c3)n2)c1] |
| 87 | 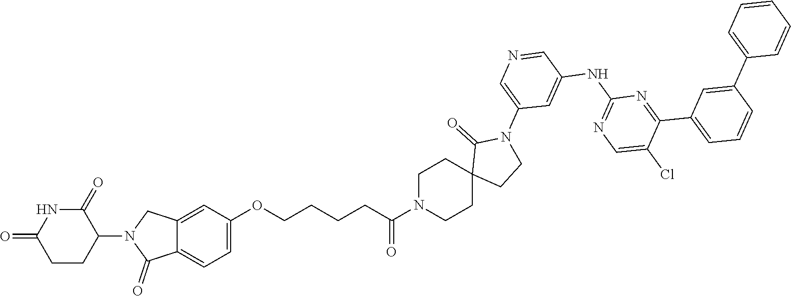 [SMILES: O=C1CCC(N2Cc3cc(OCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 88 | 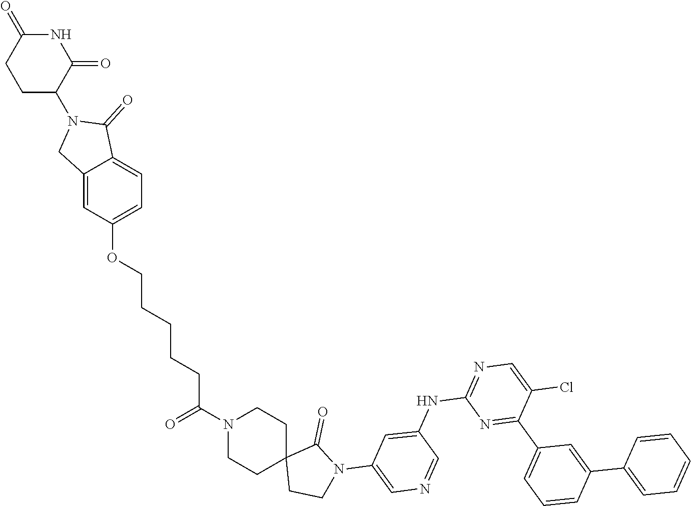 [SMILES: O=C1CCC(N2Cc3cc(OCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1] |
| 89 | 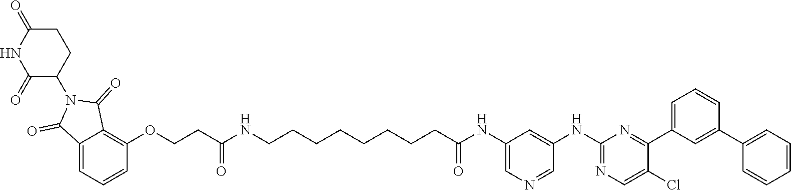 [SMILES: O=C(CCOc1cccc2c1C(=O)N(C1CCC(=O)NC1=O)C2=O)NCCCCCCCCC(=O)Nc1cncc(Nc2ncc(Cl)c(-c3cccc(-c4ccccc4)c3)n2)c1] |
| 91 | 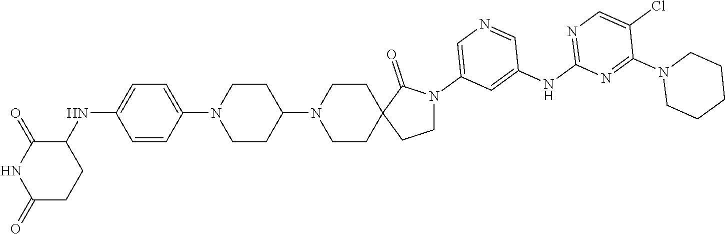 [SMILES: O=C1CCC(Nc2ccc(N3CCC(N4CCC5(CCN(c6cncc(Nc7ncc(Cl)c(N8CCCCC8)n7)c6)C5=O)CC4)CC3)cc2)C(=O)N1] |
| 166 | 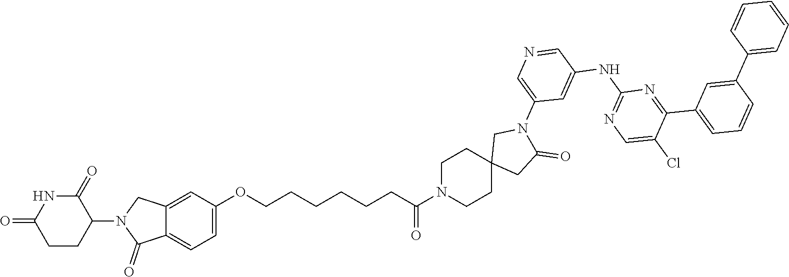 [SMILES: O=C1CCC(N2Cc3cc(OCCCCCCC(=O)N4CCC5(CC4)CC(=O)N(c4cncc(Nc6ncc(Cl)c(-c7cccc(-c8ccccc8)c7)n6)c4)C5)ccc3C2=O)C(=O)N1] |
| 167 | 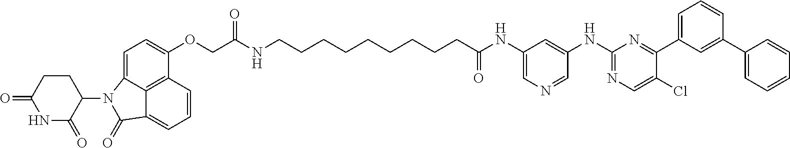 [SMILES: O=C(COc1ccc2c3c(cccc13)C(=O)N2C1CCC(=O)NC1=O)NCCCCCCCCCC(=O)Nc1cncc(Nc2ncc(Cl)c(-c3cccc(-c4ccccc4)c3)n2)c1] |
| 168 | 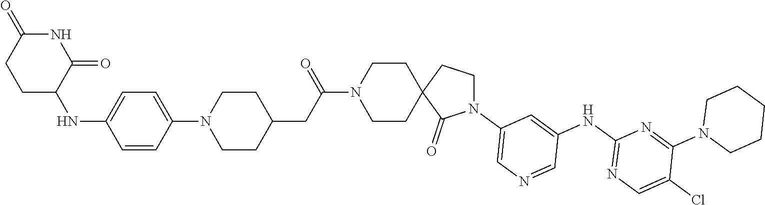 [SMILES: O=C1CCC(Nc2ccc(N3CCC(CC(=O)N4CCC5(CC4)CCN(c4cncc(Nc6ncc(Cl)c(N7CCCCC7)n6)c4)C5=O)CC3)cc2)C(=O)N1] |
| 169 | 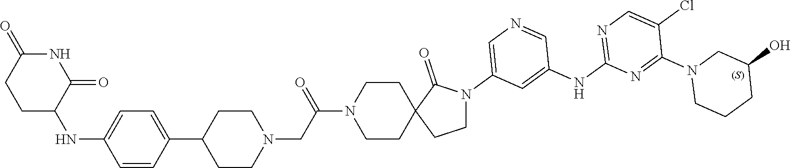 |
| 170 | 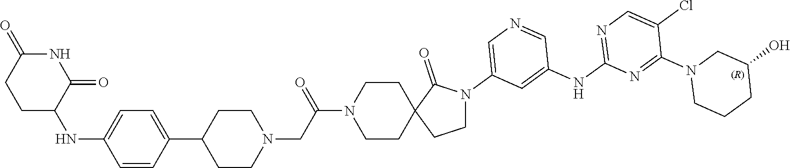 |
| 171 | 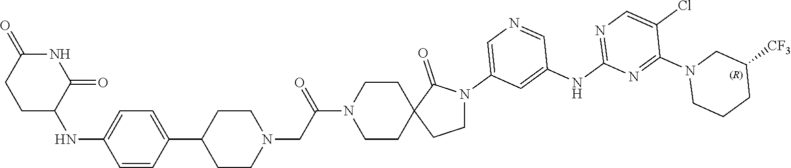 |
| 172 | 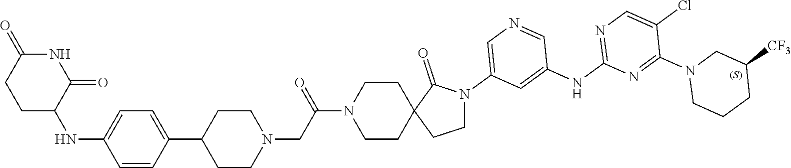 |
| 173 | 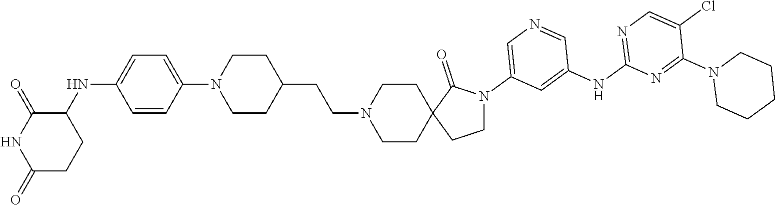 [SMILES: O=C1CCC(Nc2ccc(N3CCC(CCN4CCC5(CC4)CCN(c4cncc(Nc6ncc(Cl)c(N7CCCCC7)n6)c4)C5=O)CC3)cc2)C(=O)N1] |
| 174 | 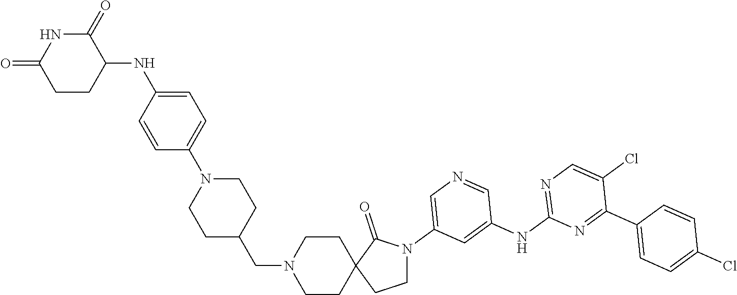 [SMILES: O=C1CCC(Nc2ccc(N3CCC(CN4CCC5(CC4)CCN(c4cncc(Nc6ncc(Cl)c(-c7ccc(Cl)cc7)n6)c4)C5=O)CC3)cc2)C(=O)N1] |
| 175 | 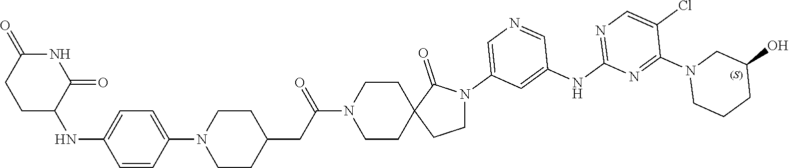 |
| 176 | 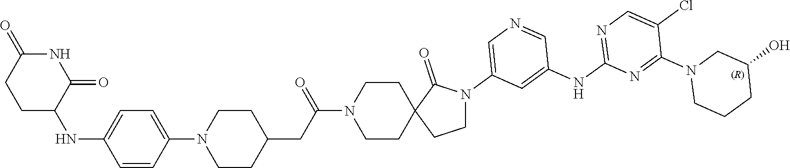 |
| 177 | 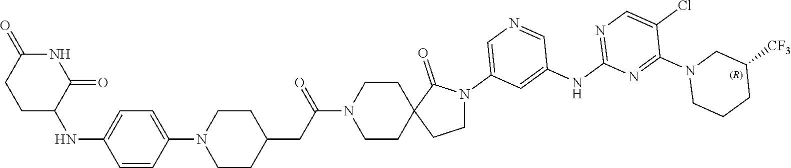 |
| 178 | 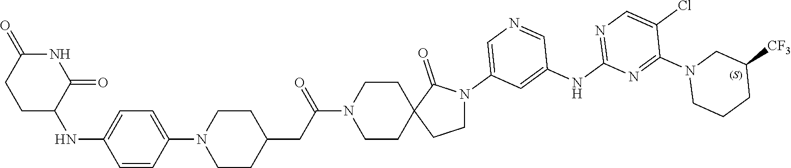 |
| 179 | 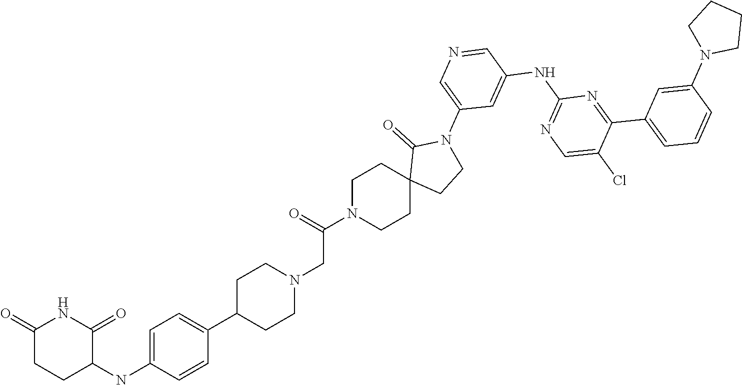 |
| 180 | 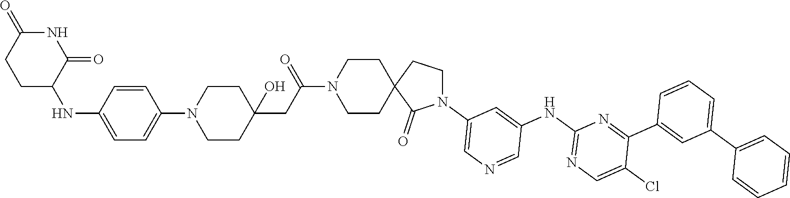 [SMILES: O=C1CCC(Nc2ccc(N3CCC(O)(CC(=O)N4CCC5(CC4)CCN(c4cncc(Nc6ncc(Cl)c(-c7cccc(-c8ccccc8)c7)n6)c4)C5=O)CC3)cc2)C(=O)N1] |
| 181 | 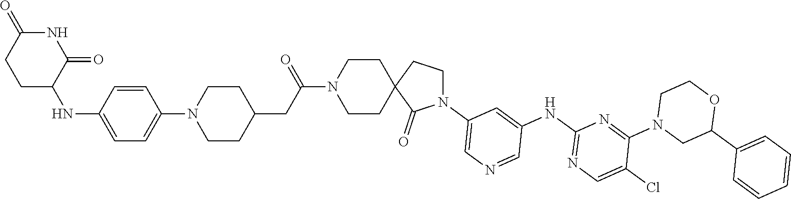 [SMILES: O=C1CCC(Nc2ccc(N3CCC(CC(=O)N4CCC5(CC4)CCN(c4cncc(Nc6ncc(Cl)c(N7CCOC(c8ccccc8)C7)n6)c4)C5=O)CC3)cc2)C(=O)N1] |
| 182 | 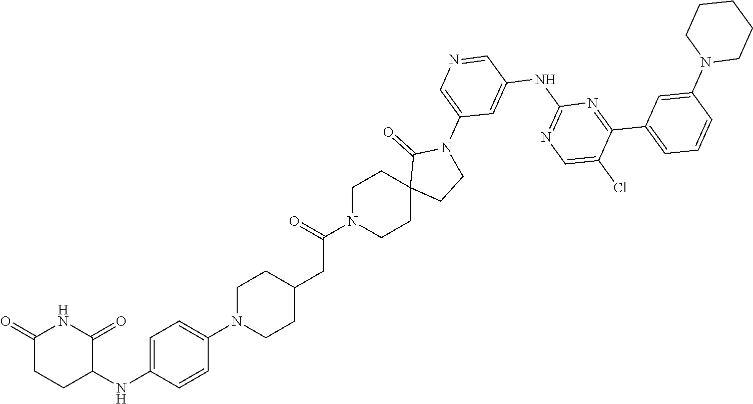 [SMILES: O=C1CCC(Nc2ccc(N3CCC(CC(=O)N4CCC5(CC4)CCN(c4cncc(Nc6ncc(Cl)c(-c7cccc(N8CCCCC8)c7)n6)c4)C5=O)CC3)cc2)C(=O)N1] |
| 183 | 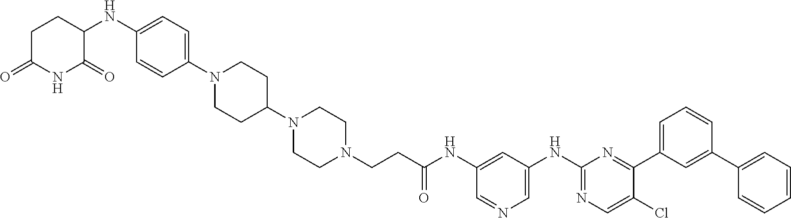 [SMILES: O=C1CCC(Nc2ccc(N3CCC(N4CCN(CCC(=O)Nc5cncc(Nc6ncc(Cl)c(-c7cccc(-c8ccccc8)c7)n6)c5)CC4)CC3)cc2)C(=O)N1] |
| 184 | 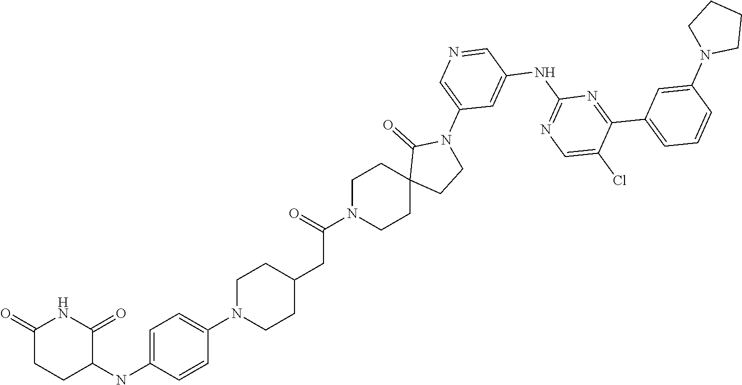 |
| 185 | 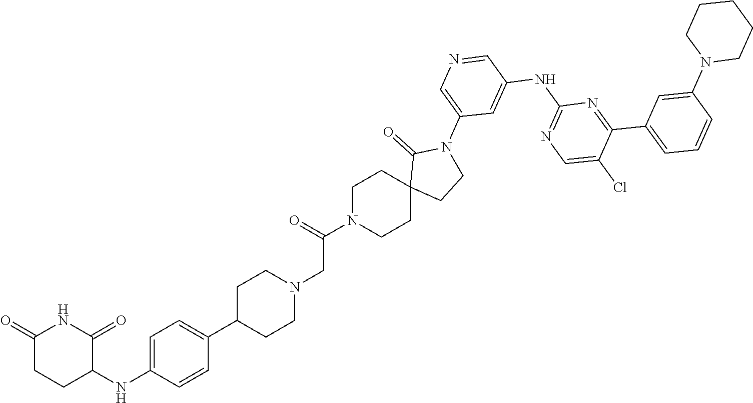 [SMILES: O=C1CCC(Nc2ccc(C3CCN(CC(=O)N4CCC5(CC4)CCN(c4cncc(Nc6ncc(Cl)c(-c7cccc(N8CCCCC8)c7)n6)c4)C5=O)CC3)cc2)C(=O)N1] |
| 186 | 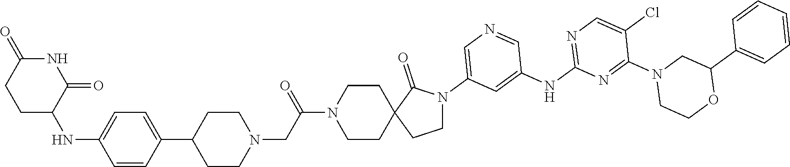 [SMILES: O=C1CCC(Nc2ccc(C3CCN(CC(=O)N4CCC5(CC4)CCN(c4cncc(Nc6ncc(Cl)c(N7CCOC(c8ccccc8)C7)n6)c4)C5=O)CC3)cc2)C(=O)N1] |
| 187 | 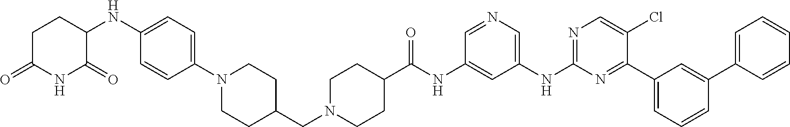 [SMILES: O=C1CCC(Nc2ccc(N3CCC(CN4CCC(C(=O)Nc5cncc(Nc6ncc(Cl)c(-c7cccc(-c8ccccc8)c7)n6)c5)CC4)CC3)cc2)C(=O)N1] |
| 188 | 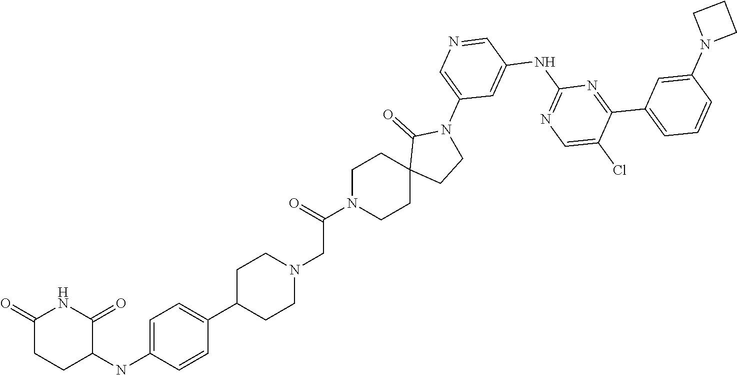 |
| 189 | 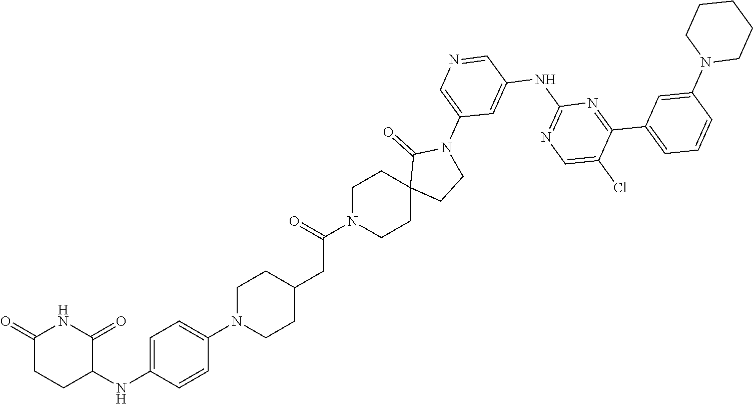 [SMILES: O=C1CCC(Nc2ccc(N3CCC(CC(=O)N4CCC5(CC4)CCN(c4cncc(Nc6ncc(Cl)c(-c7cccc(N8CCCCC8)c7)n6)c4)C5=O)CC3)cc2)C(=O)N1] |
| 190 | 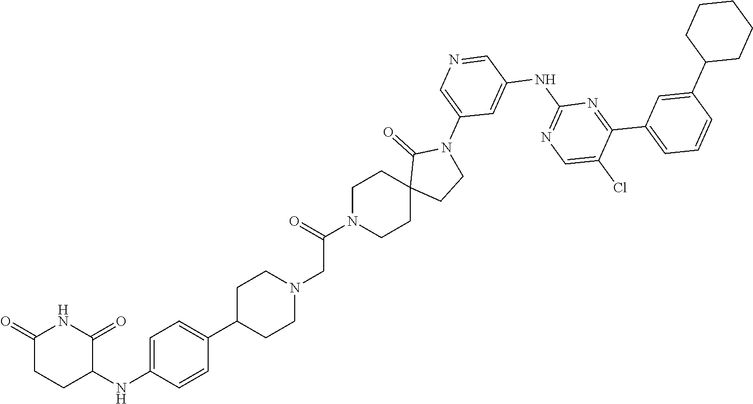 [SMILES: O=C1CCC(Nc2ccc(C3CCN(CC(=O)N4CCC5(CC4)CCN(c4cncc(Nc6ncc(Cl)c(-c7cccc(C8CCCCC8)c7)n6)c4)C5=O)CC3)cc2)C(=O)N1] |
| 191 | 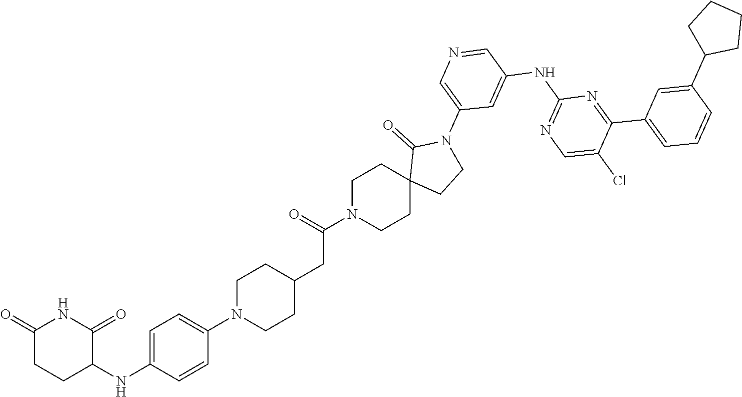 [SMILES: O=C1CCC(Nc2ccc(N3CCC(CC(=O)N4CCC5(CC4)CCN(c4cncc(Nc6ncc(Cl)c(-c7cccc(C8CCCC8)c7)n6)c4)C5=O)CC3)cc2)C(=O)N1] |
| 192 | 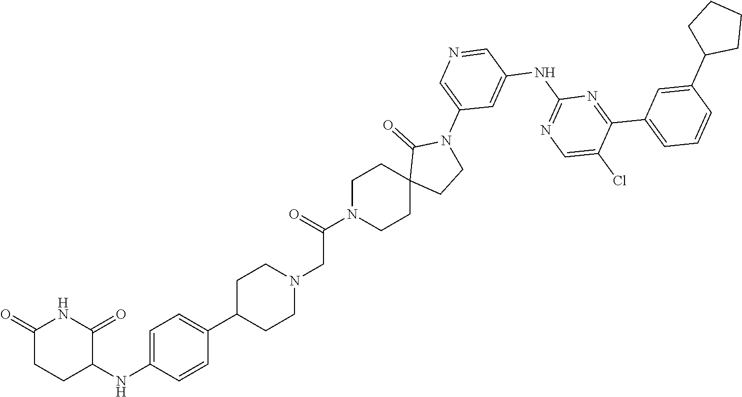 [SMILES: O=C1CCC(Nc2ccc(C3CCN(CC(=O)N4CCC5(CC4)CCN(c4cncc(Nc6ncc(Cl)c(-c7cccc(C8CCCC8)c7)n6)c4)C5=O)CC3)cc2)C(=O)N1] |
| 193 | 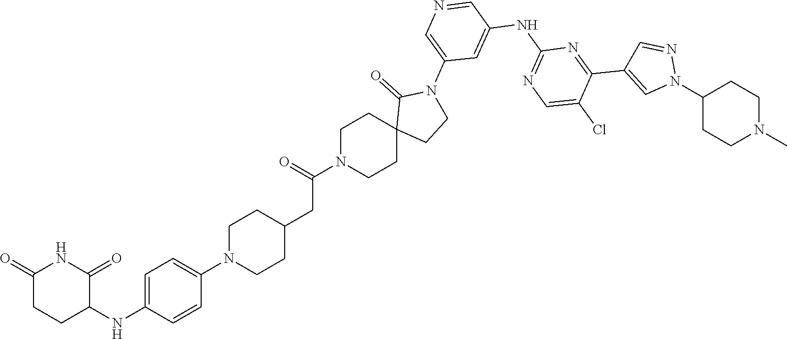 [SMILES: CN1CCC(n2cc(-c3nc(Nc4cncc(N5CCC6(CCN(C(=O)CC7CCN(c8ccc(NC9CCC(=O)NC9=O)cc8)CC7)CC6)C5=O)c4)ncc3Cl)cn2)CC1] |
| 194 | 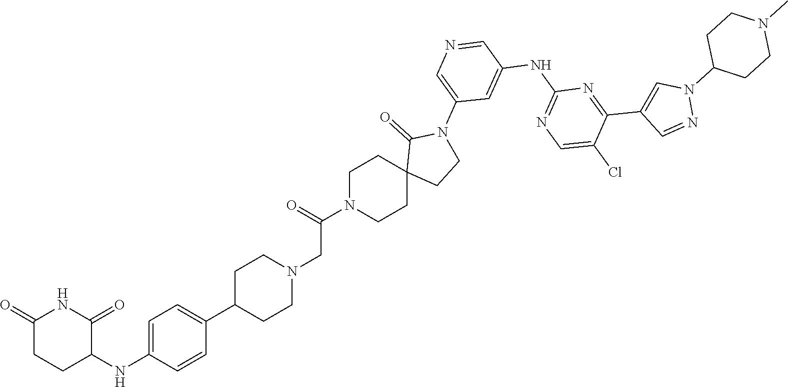 [SMILES: CN1CCC(n2cc(-c3nc(Nc4cncc(N5CCC6(CCN(C(=O)CN7CCC(c8ccc(NC9CCC(=O)NC9=O)cc8)CC7)CC6)C5=O)c4)ncc3Cl)cn2)CC1] |
| 195 | 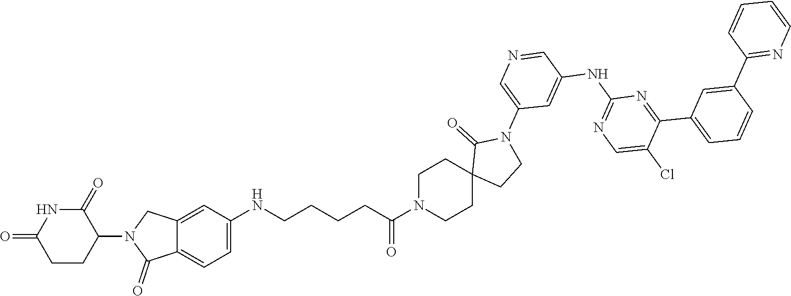 [SMILES: O=C1CCC(N2Cc3cc(NCCCCC(=O)N4CCC5(CC4)CCN(c4cncc(Nc6ncc(Cl)c(-c7cccc(-c8ccccn8)c7)n6)c4)C5=O)ccc3C2=O)C(=O)N1] |
| 196 | 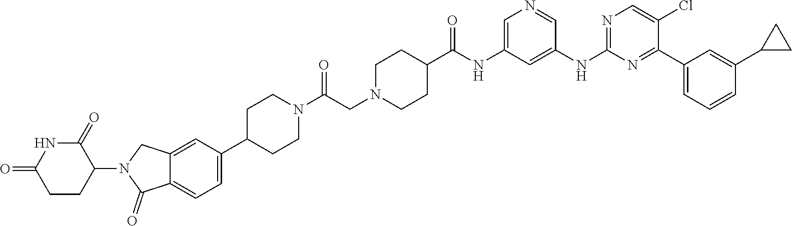 [SMILES: O=C1CCC(N2Cc3cc(C4CCN(C(=O)CN5CCC(C(=O)Nc6cncc(Nc7ncc(Cl)c(-c8cccc(C9CC9)c8)n7)c6)CC5)CC4)ccc3C2=O)C(=O)N1] |
| 197 | 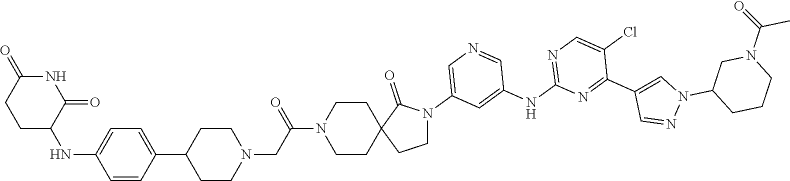 [SMILES: CC(=O)N1CCCC(n2cc(-c3nc(Nc4cncc(N5CCC6(CCN(C(=O)CN7CCC(c8ccc(NC9CCC(=O)NC9=O)cc8)CC7)CC6)C5=O)c4)ncc3Cl)cn2)C1] |
| 198 | 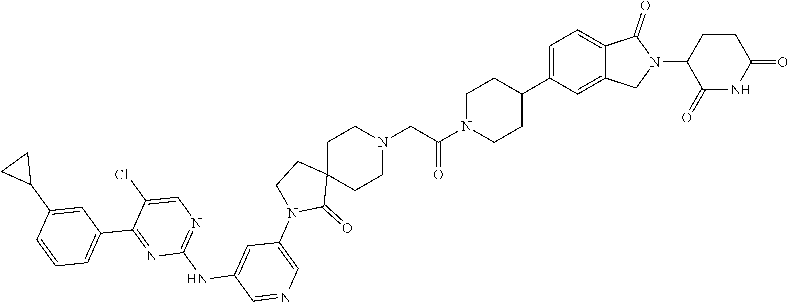 [SMILES: O=C1CCC(N2Cc3cc(C4CCN(C(=O)CN5CCC6(CC5)CCN(c5cncc(Nc7ncc(Cl)c(-c8cccc(C9CC9)c8)n7)c5)C6=O)CC4)ccc3C2=O)C(=O)N1] |
| 199 | 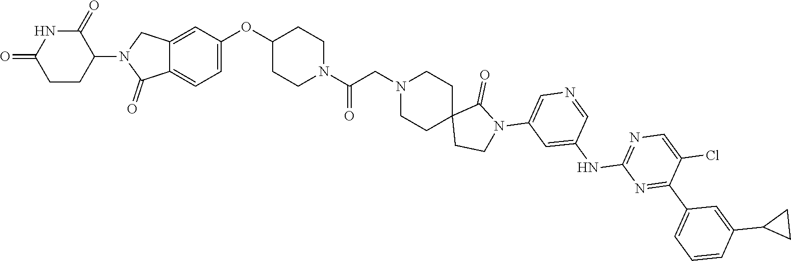 [SMILES: O=C1CCC(N2Cc3cc(OC4CCN(C(=O)CN5CCC6(CC5)CCN(c5cncc(Nc7ncc(Cl)c(-c8cccc(C9CC9)c8)n7)c5)C6=O)CC4)ccc3C2=O)C(=O)N1] |
| 200 | 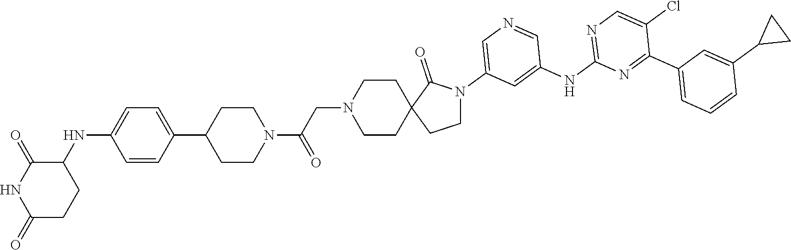 [SMILES: O=C1CCC(Nc2ccc(C3CCN(C(=O)CN4CCC5(CC4)CCN(c4cncc(Nc6ncc(Cl)c(-c7cccc(C8CC8)c7)n6)c4)C5=O)CC3)cc2)C(=O)N1] |
| 201 | 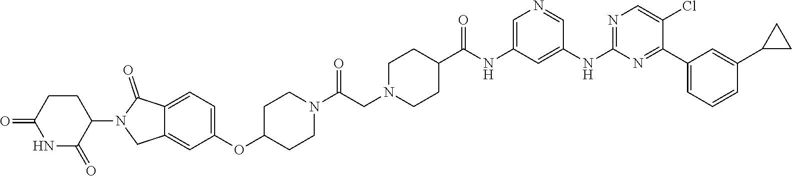 [SMILES: O=C1CCC(N2Cc3cc(OC4CCN(C(=O)CN5CCC(C(=O)Nc6cncc(Nc7ncc(Cl)c(-c8cccc(C9CC9)c8)n7)c6)CC5)CC4)ccc3C2=O)C(=O)N1] |
| 202 | 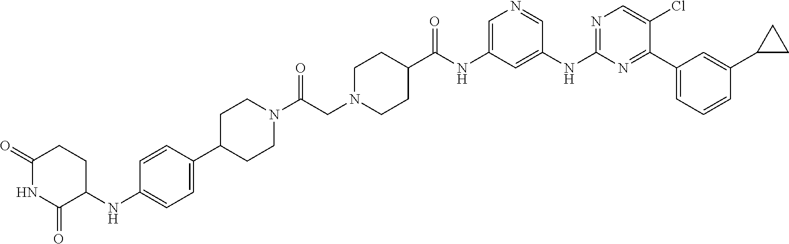 [SMILES: O=C1CCC(Nc2ccc(C3CCN(C(=O)CN4CCC(C(=O)Nc5cncc(Nc6ncc(Cl)c(-c7cccc(C8CC8)c7)n6)c5)CC4)CC3)cc2)C(=O)N1] |
| 203 | 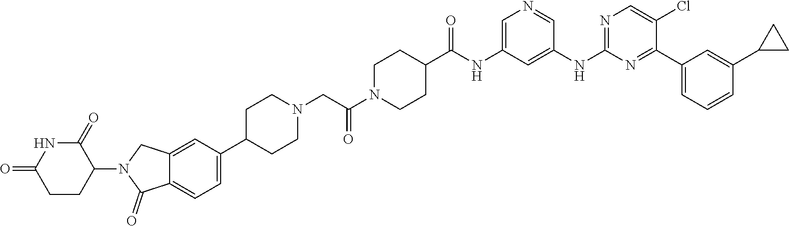 [SMILES: O=C1CCC(N2Cc3cc(C4CCN(CC(=O)N5CCC(C(=O)Nc6cncc(Nc7ncc(Cl)c(-c8cccc(C9CC9)c8)n7)c6)CC5)CC4)ccc3C2=O)C(=O)N1] |
| 204 | 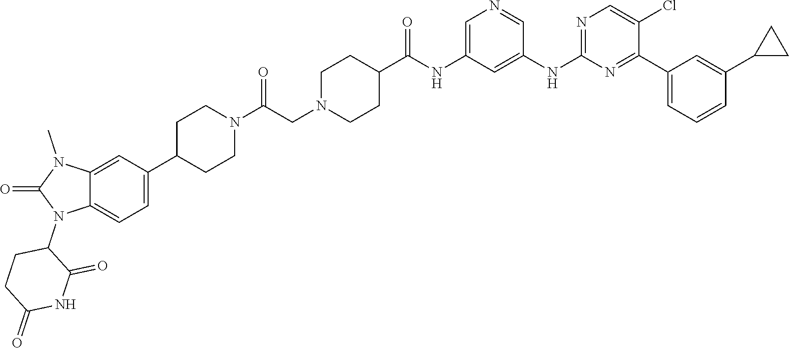 [SMILES: Cn1c(=O)n(C2CCC(=O)NC2=O)c2ccc(C3CCN(C(=O)CN4CCC(C(=O)Nc5cncc(Nc6ncc(Cl)c(-c7cccc(C8CC8)c7)n6)c5)CC4)CC3)cc21] |
| 205 | 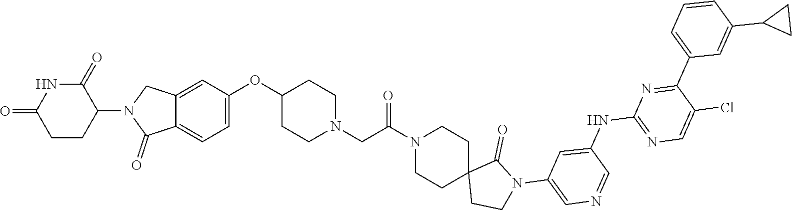 [SMILES: O=C1CCC(N2Cc3cc(OC4CCN(CC(=O)N5CCC6(CC5)CCN(c5cncc(Nc7ncc(Cl)c(-c8cccc(C9CC9)c8)n7)c5)C6=O)CC4)ccc3C2=O)C(=O)N1] |
| 206 | 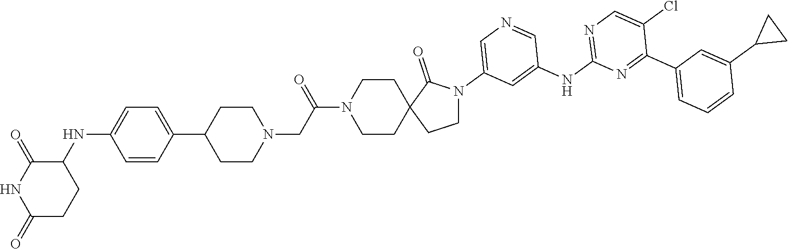 [SMILES: O=C1CCC(Nc2ccc(C3CCN(CC(=O)N4CCC5(CC4)CCN(c4cncc(Nc6ncc(Cl)c(-c7cccc(C8CC8)c7)n6)c4)C5=O)CC3)cc2)C(=O)N1] |
| 207 | 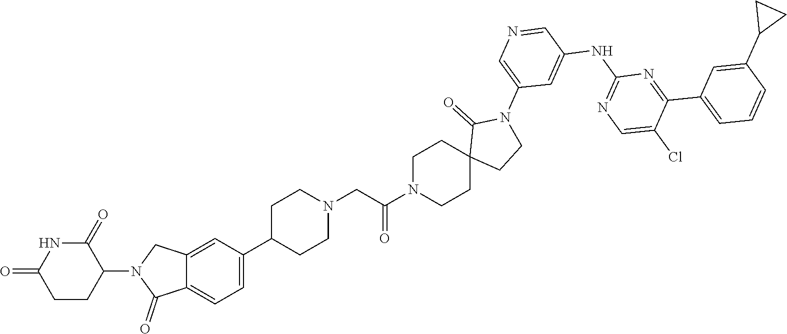 [SMILES: O=C1CCC(N2Cc3cc(C4CCN(CC(=O)N5CCC6(CC5)CCN(c5cncc(Nc7ncc(Cl)c(-c8cccc(C9CC9)c8)n7)c5)C6=O)CC4)ccc3C2=O)C(=O)N1] |
| 208 | 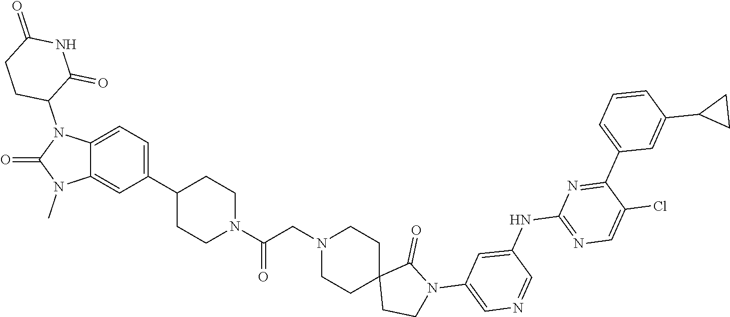 [SMILES: Cn1c(=O)n(C2CCC(=O)NC2=O)c2ccc(C3CCN(C(=O)CN4CCC5(CC4)CCN(c4cncc(Nc6ncc(Cl)c(-c7cccc(C8CC8)c7)n6)c4)C5=O)CC3)cc21] |
| 209 | 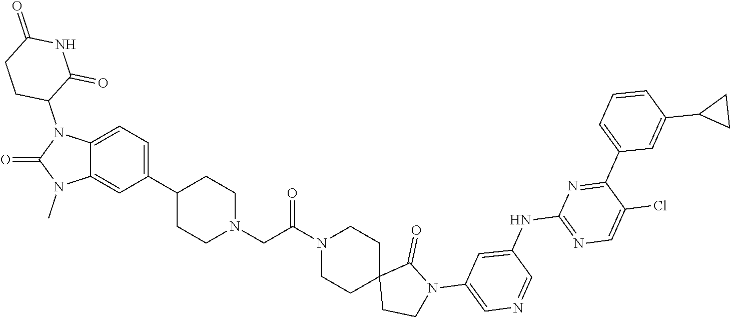 [SMILES: Cn1c(=O)n(C2CCC(=O)NC2=O)c2ccc(C3CCN(CC(=O)N4CCC5(CC4)CCN(c4cncc(Nc6ncc(Cl)c(-c7cccc(C8CC8)c7)n6)c4)C5=O)CC3)cc21] |
| 210 | 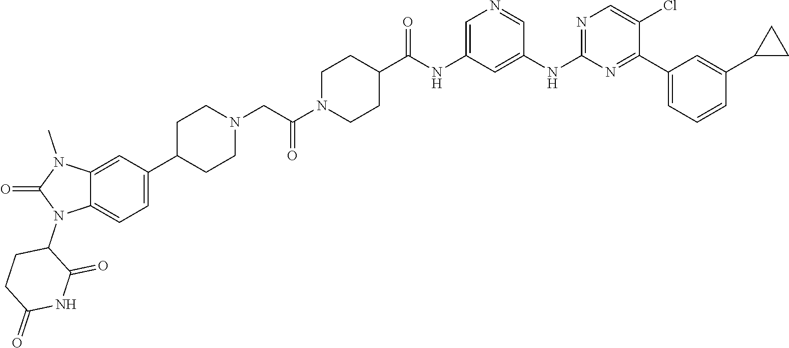 [SMILES: Cn1c(=O)n(C2CCC(=O)NC2=O)c2ccc(C3CCN(CC(=O)N4CCC(C(=O)Nc5cncc(Nc6ncc(Cl)c(-c7cccc(C8CC8)c7)n6)c5)CC4)CC3)cc21] |
| 211 | 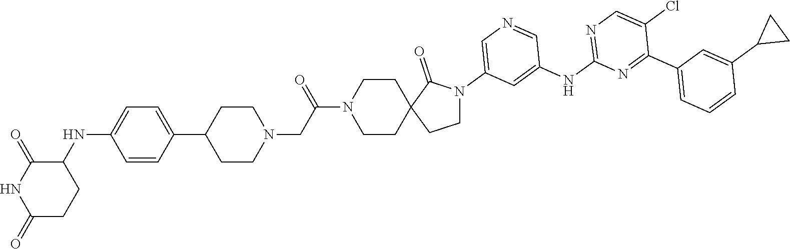 [SMILES: O=C1CCC(Nc2ccc(C3CCN(CC(=O)N4CCC5(CC4)CCN(c4cncc(Nc6ncc(Cl)c(-c7cccc(C8CC8)c7)n6)c4)C5=O)CC3)cc2)C(=O)N1] |
| 212 | 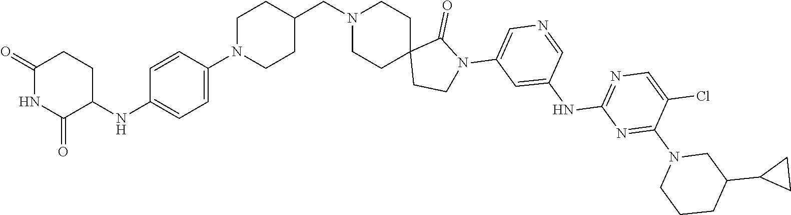 [SMILES: O=C1CCC(Nc2ccc(N3CCC(CN4CCC5(CC4)CCN(c4cncc(Nc6ncc(Cl)c(N7CCCC(C8CC8)C7)n6)c4)C5=O)CC3)cc2)C(=O)N1] |

### Pharmaceutical Compositions

Another aspect of the present invention is directed to a pharmaceutical composition that comprises a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. In some embodiments, the pharmaceutical composition comprises a therapeutically effective amount of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients. Representative pharmaceutically acceptable carriers are disclosed in, e.g., Remington: The Science and Practice of Pharmacy (20th ed.), ed. A. R. Gennaro, Lippincott Williams & Wilkins, 2000; Encyclopedia of Pharmaceutical Technology, eds. J. Swarbrick and J. C. Boylan, 1988-1999, Marcel Dekker, New York; and Handbook of Pharmaceutical Excipients (3rd ed.) ed. A. H. Kibbe, Amer. Pharm. Assoc., 2000; each of which is incorporated herein by reference in its entirety).

Compounds of Formula (I′) or (I) or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York, each of which is incorporated herein by reference in its entirety). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal).

In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of Formula (I′) or (I) or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions comprising liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of Formula (I′) or (I) or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of Formula (I′) or (I) or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of Formula (I′) or (I) or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated comprising a powder mix of the compound and a suitable powder base such as lactose or starch.

Compounds of Formula (I′) or (I) or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, e.g., Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997), each of which is incorporated herein by reference in its entirety. Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allantoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of Formula (I′) or (I), or any related formula such as (I-a), (I-b), (I-c), (J-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing; or a composition including a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, effective in producing the desired therapeutic response in a particular patient in need thereof. Therefore, the term “therapeutically effective amount” includes the amount of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer, autophagy-dependent disease (e.g., neurodegenerative disorder)) cells, or reduces the amount of at least one other of PIP4K2A, PIP4K2B, and PIP4K2C in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, e.g., Goodman and Gilman's The Pharmacological Basis of Therapeutics, 10th Edition, A. Gilman, J. Hardman and L. Limbird, eds., McGraw-Hill Press, 155-173, 2001), which is incorporated herein by reference in its entirety.

### Methods of Use

In some aspects, the present invention is directed to methods of treating diseases or disorders comprising reducing the level or activity of at least one of PIP4K2A, PIP4K2B, and PIP4K2C, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some aspects, the present invention is directed to methods of treating diseases or disorders comprising reducing the level or activity of at least one of PIP4K2A, PIP4K2B, and PIP4K2C, comprises administration of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to a subject in need thereof. In some aspects, the present invention is directed to methods of treating diseases or disorders comprising reducing the level or activity of at least one of PIP4K2A, PIP4K2B, and PIP4K2C, comprising administration of (a) a therapeutically effective amount of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a therapeutically effective amount of compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to a subject in need thereof. In some aspects, the present invention is directed to methods of treating diseases or disorders comprising reducing the level or activity of at least one of PIP4K2A, PIP4K2B, and PIP4K2C, comprising administration of a therapeutically effective amount of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to a subject in need thereof.

In some embodiments, the level or activity of at least one of PIP4K2A, PIP4K2B, and PIP4K2C in the subject is reduced by, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to an untreated control subject.

A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The term “subject” (or “patient”) as used herein includes all members of the animal kingdom prone to or suffering from the indicated disease or disorder. In some embodiments, the subject is a mammal, e.g., a human or a non-human mammal. In some embodiments, the subject is a human. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Degradation or depletion of PIP4K2A, PIP4K2B, and/or PIP4K2C proteins is useful for treating and/or diagnosing cancer. In some cases, the PIP4K2C protein is targeted because it is expressed at higher levels in immune cells than the PIP4K2A and PIP4K2B proteins. Degradation or depletion of the PI1P4K2A protein, the PIP4K2B protein, and/or particularly the PIP4K2C protein can enhance immune responses against cancer and tumors. Depletion or degradation of PIP4Ks, particularly PIP4K2B protein, is useful for treatment of insulin resistance.

In some embodiments, the compounds or compositions comprising such compounds may be used to reduce the level of PIP4K2B in a patient suffering from or suspected of suffering from insulin resistance. In some embodiments, the compounds may be used to decrease the level of PIP4K2C in a patient suffering from or suspected of suffering from cancer, immune deficiency, autoimmune disease, or infectious disease, or a combination thereof.

In some aspects, the present invention is directed to methods of enhancing immune function in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some aspects, the present invention is directed to methods of enhancing immune function in a subject in need thereof, comprising administering to a subject a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing. In some aspects, the present invention is directed to methods of enhancing immune function in a subject in need thereof, comprising administering to a subject a therapeutically effective amount of (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some aspects, the present invention is directed to methods of enhancing immune functions in a subject in need thereof, comprising administering to a subject a therapeutically effective amount of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing.

In some embodiments, immune functions in the subject are enhanced by, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to an untreated control subject.

In some aspects, the present invention is directed to methods of stimulating/activating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some aspects, the present invention is directed to methods of stimulating/activating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing. In some embodiments, the present invention is directed to methods of stimulating/activating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of stimulating/activating the immune system comprising reducing scaffolding or interaction of at least one of PIP4K2A, PIP4K2B, and PIP4K2C with at least one other of PIP4K2A, PIP4K2B, and PIP4K2C or phosphatidylinositol-4-phosphate 5-kinase (PIP5K) in a subject in need thereof, comprising administering to a subject a therapeutically effective amount of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing. The reduction in the scaffolding or interaction may stimulate the immune system.

In some embodiments, compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be useful in the treatment of viral infection. In some embodiments, the present invention is directed to methods of treating a viral infection in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating a viral infection in a subject in need thereof, comprising administering a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject. In some embodiments, compounds of Formula (I′) or (I), or any related formula such as (I-a), (I-b), (I-c), (J-d), (I-e), (I-f), (II), (II-a), (II-b), (II-c), (JJ-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be useful in the treatment of bacterial infection (e.g., necrotizing soft tissue infection). In some embodiments, the present invention is directed to methods of treating a bacterial infection in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating a bacterial infection in a subject in need thereof, comprising administering a compound of Formula (I′) or (I), or any related formula such as (I-a), (I-b), (I-c), (T-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject. In some embodiments, the bacterial infection comprises necrotizing soft tissue infection.

In some embodiments, compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be useful in the treatment of a disease or disorder associated with at least one of PIP4K2A, PIP4K2B, and PIP4K2C. In some embodiments, the present invention is directed to methods of treating a disease or disorder associated with at least one of PIP4K2A, PIP4K2B, and PIP4K2C in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating a disease or disorder associated with at least one of PIP4K2A, PIP4K2B, and PIP4K2C in a subject in need thereof, comprising administering a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject. In some embodiments, the disease or disorder is associated with PIP4K2C. In some embodiments, compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be useful in the treatment of a disease or disorder associated with PIP4K2C.

In some embodiments, compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be useful in the treatment of cell proliferative diseases and disorders (e.g., cancer or benign neoplasms). In some embodiments, the present invention is directed to methods of treating a cell proliferative disease or disorder in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating a cell proliferative disease or disorder in a subject in need thereof, comprising administering a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject. As used herein, the term “cell proliferative disease or disorder” refers to the conditions characterized by deregulated or abnormal cell growth, or both, including noncancerous conditions such as neoplasms, precancerous conditions, benign tumors, and cancer.

Exemplary types of non-cancerous (e.g., cell proliferative) diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, metabolic diseases, and allergic and genetic diseases.

In some embodiments, the compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be useful in the treatment of neurodegenerative diseases and disorders. In some embodiments, the present invention is directed to methods of treating a neurodegenerative disease or disorder in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating a neurodegenerative disease or disorder in a subject in need thereof, comprising administering a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the compounds of Formula (I′) or (I), or any related formula such as (I-a), (I-b), (I-c), (J-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be useful in the treatment of autoimmune diseases and disorders. In some embodiments, the present invention is directed to methods of treating an autoimmune disease or disorder in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating an autoimmune disease or disorder in a subject in need thereof, comprising administering a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject. As used herein, the term “autoimmune disease” refers to conditions where the immune system produces antibodies that attack normal body tissues. Representative examples of such diseases include autoimmune hematological disorders (e.g., hemolytic anemia, aplastic anemia, anhidrotic ectodermal dysplasia, pure red cell anemia and idiopathic thrombocytopenia), Sjogren's syndrome, Hashimoto thyroiditis, rheumatoid arthritis, juvenile (type 1) diabetes, polymyositis, scleroderma, Addison's disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, autoimmune uveoretinitis, lichen planus, bullous pemphigus, pemphigus vulgaris, pemphigus foliaceus, paraneoplastic pemphigus, myasthenia gravis, immunoglobulin A nephropathy, Wegener granulomatosis, autoimmune oophoritis, sarcoidosis, rheumatic carditis, ankylosing spondylitis, Grave's disease, autoimmune thrombocytopenic purpura, psoriasis, psoriatic arthritis, dermatitis herpetiformis, ulcerative colitis, and temporal arteritis.

In other embodiments, the methods are directed to treating subjects having cancer. Compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. In some embodiments, the present invention is directed to methods of treating a carcinoma, sarcoma, melanoma, or hematological cancer in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating carcinoma, sarcoma, melanoma, or hematological cancer in a subject in need thereof, comprising administering a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer (e.g., triple negative breast cancer), bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colorectal carcinoma, colorectal carcinoma with microsatellite instability (MSI), colorectal carcinoma that is microsatellite stable (MSS), colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., gastric cancer, stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, Merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, pancreatic ductal adenocarcinoma, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma (e.g., head and neck squamous cell carcinoma), testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer). In some embodiments, the present invention is directed to methods of treating a sarcoma in a subject in need thereof, comprising administering (a) a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, or (b) a pharmaceutical composition comprising a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, and a pharmaceutically acceptable carrier, to the subject. In some embodiments, the present invention is directed to methods of treating sarcoma in a subject in need thereof, comprising administering a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, to the subject.

In some embodiments, methods of the present invention entail treatment of subjects having cell proliferative diseases or disorders of the hematological system, liver, brain, lung, colon, pancreas, prostate, ovary, breast, skin and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, an compound may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the cancer is a hematological cancer e.g., leukemia, lymphoma or multiple myeloma.

The compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, the compounds may be administered to a patient who has received another therapy, such as chemotherapy, radioimmunotherapy, surgical therapy, immunotherapy, radiation therapy, targeted therapy or any combination thereof.

The methods of the present invention may entail administration of a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Combination Therapy

The compounds of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of Formula (I′) or (I), or any related formula such as (I-a), (I-b), (I-c), (J-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing, in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Therapeutics, 10th ed., McGraw-Hill, New York, 2001; Physician's Desk Reference, 60th ed., 2006, which is incorporated herein by reference in its entirety. For example, anti-cancer agents that may be suitable for use in combination with the compounds are known in the art. See, e.g., U.S. Pat. No. 9,101,622 (Section 5.2 thereof) and U.S. Pat. No. 9,345,705 B2 (Columns 12-18 thereof), each of which is incorporated herein by reference in its entirety. Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, a compound of Formula (I′) or (I), or any related formula such as (I-a), (I-b), (I-c), (J-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The compounds and their pharmaceutically acceptable salts and stereoisomers and/or compositions comprising them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutical composition thereof. The kits or pharmaceutical systems of the invention may also include printed instructions for using the compounds and compositions. In one aspect, a kit comprises a compound of the disclosure or a pharmaceutically acceptable salt thereof, and a label and/or instructions for use of the compound in the treatment of a disease or disorder described herein. The kit may comprise a unit dosage form of the compound.

### ENUMERATED EMBODIMENTS

The following enumerated embodiments are representative of some aspects of the invention.
Enumerated Embodiment 1. A compound of Formula (I)

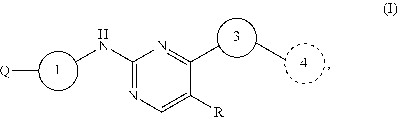

or a tautomer or stereoisomer thereof, or a pharmaceutically acceptable salt of any of the foregoing, wherein:
R is halo;
Ring 1 is pyridyl optionally substituted with one or more R¹;
Ring 3 is a monocyclic ring selected from the group consisting of 3- to 6-membered cycloalkyl optionally substituted with one or more R^{3a}, 4- to 6-membered heterocyclyl optionally substituted with one or more R^{3a}, phenyl optionally substituted with one or more R^{3b}, or 5- to 6-membered heteroaryl optionally substituted with one or more R^{3b};
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Q is Ring 5, wherein Ring 5 is a pyrrolidinone or imidazolidinone, each of which is optionally substituted with one or more R⁵, or
Q is of formula i)

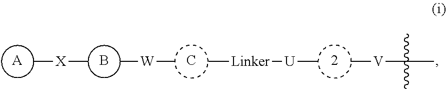

wherein:
Ring A is selected from the group consisting of

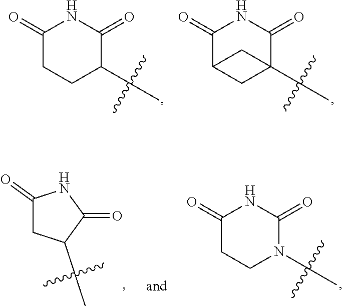 [SMILES: *C1CCC(=O)NC1=O ; *C12CC(C1)C(=O)NC2=O ; *C1CC(=O)NC1=O ; *N1CCC(=O)NC1=O]

each of which is optionally substituted with C₁₋₄alkyl or halo;
X is a bond, —O—, —NH—, or —NHC(O)—;
Ring B is selected from the group consisting of

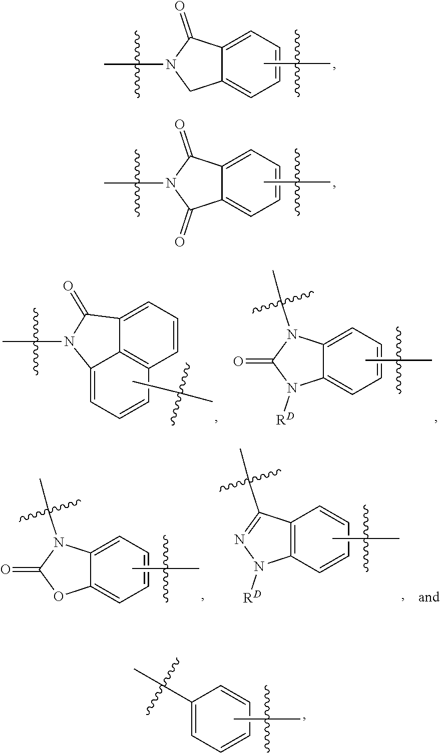

which is optionally substituted with one or more halo, C₁₋₄alkyl, C₁₋₄haloalkyl, or OH, wherein R^{D} is H or C₁₋₄alkyl;
W is a bond, —O—, —NH—, or —NHC(O)—;
Ring C is optional, and when present is 4- to 12-membered heterocyclyl optionally substituted with one or more R^{C};
Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
   * indicates attachment to Ring C when Ring C is present, and * indicates attachment to W when Ring C is absent;
   ** indicates attachment to U when Ring 2 is present, and ** indicates attachment to V when Ring 2 is absent;
   L¹ is a bond or C₁₋₆ alkylene;
   L² is a 3- to 10-membered cycloalkyl or 4- to 12-membered heterocyclyl optionally substituted with one or more R^{L}; and
   L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
U is absent when Ring 2 is absent, and is a bond or C(O) when Ring 2 is present;
Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
V is a bond, —NR^{V}—, —C(O)—, —C(O)NH—, or —NHC(O)—;
each R¹ is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R⁵ is independently C₁₋₄alkyl, or two R⁵ are taken together with the atom(s) to which they are attached to form a four- to six-membered ring which is optionally substituted with C₁₋₄alkyl or C(O)C₁₋₄alkyl;
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{V} is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
provided that, when Ring 3 and Ring 4 are each unsubstituted phenyl and Q is Ring 5, Ring 5 is substituted with one or more R⁵.
Enumerated Embodiment 2. The compound of Enumerated Embodiment 1, wherein R is chloro.
Enumerated Embodiment 3. The compound of Enumerated Embodiment 1, wherein R is fluoro.
Enumerated Embodiment 4. The compound of any one of Enumerated Embodiments 1-3, wherein Ring 1 is

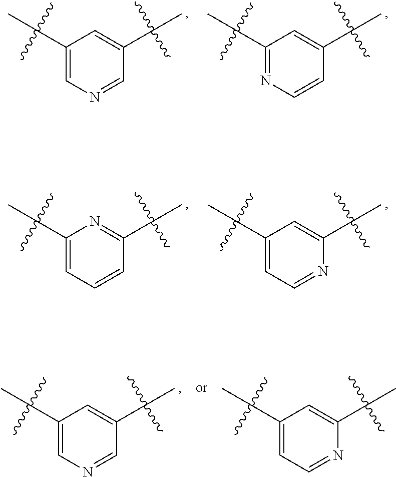 [SMILES: *c1cncc(*)c1 ; *c1ccnc(*)c1 ; *c1cccc(*)n1 ; *c1ccnc(*)c1 ; *c1cncc(*)c1 ; *c1ccnc(*)c1]

each of which is optionally substituted with one or more R¹.
Enumerated Embodiment 5. The compound of any one of Enumerated Embodiments 1-4, wherein Ring 1 is

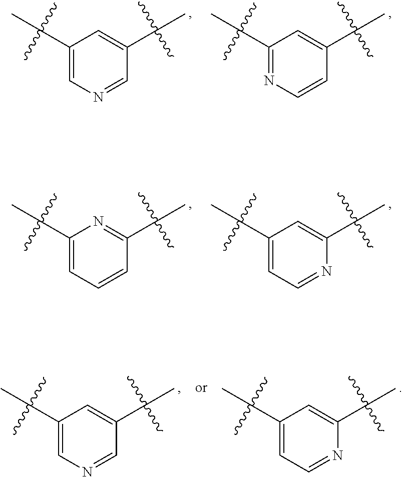 [SMILES: *c1cncc(*)c1 ; *c1ccnc(*)c1 ; *c1cccc(*)n1 ; *c1ccnc(*)c1 ; *c1cncc(*)c1 ; *c1ccnc(*)c1]

Enumerated Embodiment 6. The compound of any one of Enumerated Embodiments 1-5, wherein Ring 1 is

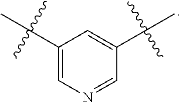 [SMILES: *c1cncc(*)c1]

Enumerated Embodiment 7. The compound of any one of Enumerated Embodiments 1-6, wherein Ring 3 is 4- to 6-membered heterocyclyl optionally substituted with one or more R^{3a}.
Enumerated Embodiment 8. The compound of Enumerated Embodiment 7, wherein Ring 3 is

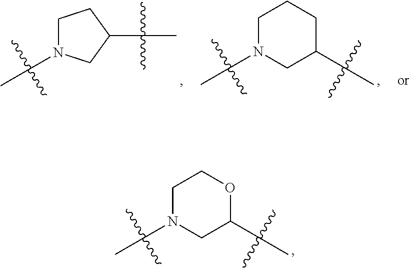 [SMILES: *C1CCN(*)C1 ; *C1CCCN(*)C1 ; *C1CN(*)CCO1]

each of which is optionally substituted with one or more R^{3a}.
Enumerated Embodiment 9. The compound of any one of Enumerated Embodiments 1-6, wherein Ring 3 is phenyl optionally substituted with one or more R^{3b}.
Enumerated Embodiment 10. The compound of any one of Enumerated Embodiments 1-6, wherein Ring 3 is 5- to 6-membered heteroaryl optionally substituted with one or more R^{3b}.
Enumerated Embodiment 11. The compound of Enumerated Embodiment 10, wherein Ring 3 is

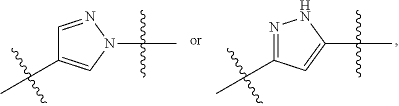 [SMILES: *c1cnn(*)c1 ; *c1cc(*)[nH]n1]

each of which is optionally substituted with one or more R^{3b}.
Enumerated Embodiment 12. The compound of any one of Enumerated Embodiments 1-11, wherein Ring 4 is absent.
Enumerated Embodiment 13. The compound of any one of Enumerated Embodiments 1-11, wherein Ring 4 is 3- to 6-membered cycloalkyl optionally substituted with one or more R^{4a}.
Enumerated Embodiment 14. The compound of any one of Enumerated Embodiments 1-11, wherein Ring 4 is 4- to 6-membered heterocyclyl optionally substituted with one or more R^{4a}.
Enumerated Embodiment 15. The compound of Enumerated Embodiment 14, wherein Ring 4 is piperidinyl or pyrrolidinyl, each of which is optionally substituted with one or more R^{4a}.
Enumerated Embodiment 16. The compound of any one of Enumerated Embodiments 1-11, wherein Ring 4 is phenyl optionally substituted with one or more R^{4b}.
Enumerated Embodiment 17. The compound of any one of Enumerated Embodiments 1-11, wherein Ring 4 is 5- to 6-membered heteroaryl optionally substituted with one or more R^{4b}.
Enumerated Embodiment 18. The compound of Enumerated Embodiment 17, wherein Ring 4 is pyridinyl or pyrazinyl, each of which is optionally substituted with one or more R^{4b}.
Enumerated Embodiment 19. The compound of any one of Enumerated Embodiments 1-18, wherein Q is Ring 5 and the compound is of Formula (II):

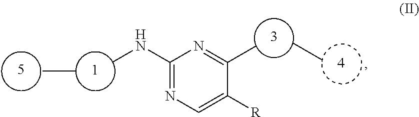

or a pharmaceutically acceptable salt thereof.
Enumerated Embodiment 20. The compound of Enumerated Embodiment 19, wherein Ring 5 is

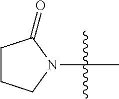 [SMILES: *N1CCCC1=O]

optionally substituted with one or more R⁵.
Enumerated Embodiment 21. The compound of Enumerated Embodiment 19 or 20, wherein Ring 5 is

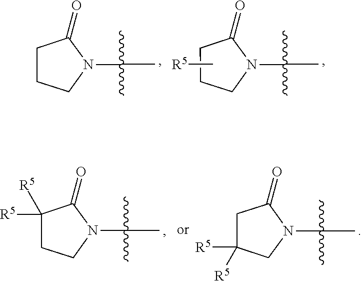

Enumerated Embodiment 22. The compound of Enumerated Embodiment 19, wherein Ring 5 is

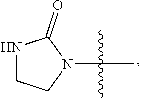 [SMILES: *N1CCNC1=O]

optionally substituted with one or more R⁵.
Enumerated Embodiment 23. The compound of any one of Enumerated Embodiments 19-22, wherein two R⁵ are taken together with the atom(s) to which they are attached to form a four- to six-membered ring which is optionally substituted with C₁₋₄alkyl or C(O)C₁₋₄alkyl.
Enumerated Embodiment 24. The compound of any one of Enumerated Embodiments 19-23, wherein two R⁵ are taken together with the atom(s) to which they are attached to form a four- to six-membered heterocyclyl ring which is optionally substituted with C₁₋₄alkyl or C(O)C₁₋₄alkyl.
Enumerated Embodiment 25. The compound of any one of Enumerated Embodiments 1-18, wherein Q is of Formula (i) and the compound is of Formula (III):

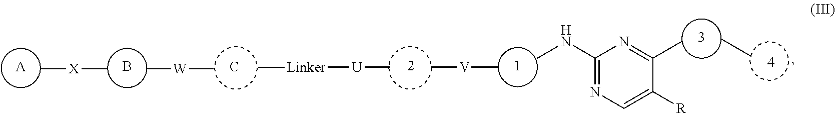

or a pharmaceutically acceptable salt thereof.
Enumerated Embodiment 26. The compound of Enumerated Embodiment 25, wherein Ring A is

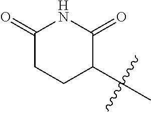 [SMILES: *C1CCC(=O)NC1=O]

optionally substituted with C₁₋₄alkyl or halo.
Enumerated Embodiment 27. The compound of Enumerated Embodiment 25 or 26, wherein Ring B is

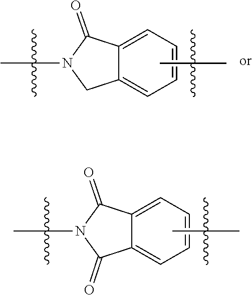

optionally substituted with one or more halo or OH.
Enumerated Embodiment 28. The compound of Enumerated Embodiment 25 or 26, wherein Ring B is

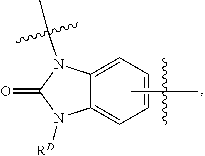

optionally substituted with one or more halo or OH, wherein R^{D} is H or C₁₋₄alkyl.
Enumerated Embodiment 29. The compound of Enumerated Embodiment 25 or 26, wherein Ring B is

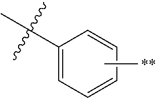

optionally substituted with one or more halo or OH.
Enumerated Embodiment 30. The compound of any one of Enumerated Embodiments 25-29, wherein Ring C is absent.
Enumerated Embodiment 31. The compound of any one of Enumerated Embodiments 25-29, wherein Ring C is piperidinyl optionally substituted with one or more R^{C}.
Enumerated Embodiment 32. The compound of any one of Enumerated Embodiments 25-29, wherein Ring C is piperazinyl optionally substituted with one or more R^{C}.
Enumerated Embodiment 33. The compound of Enumerated Embodiment 25, wherein Ring A is

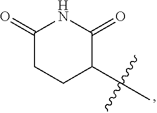 [SMILES: *C1CCC(=O)NC1=O ; ","]

X is a bond, Ring B is

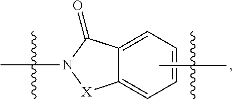

W is —O—, and Ring C is absent.
Enumerated Embodiment 34. The compound of Enumerated Embodiment 25, wherein Ring A is

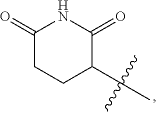 [SMILES: *C1CCC(=O)NC1=O ; ","]

X is a bond, Ring B is

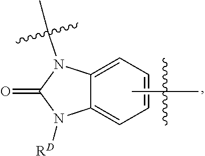

W is —O—, and Ring C is absent.
Enumerated Embodiment 35. The compound of Enumerated Embodiment 25, wherein Ring A is

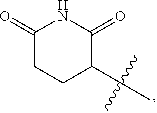 [SMILES: *C1CCC(=O)NC1=O ; ","]

X is —NH—, Ring B is

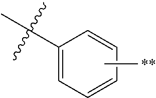

W is —O—, and Ring C is absent.
Enumerated Embodiment 36. The compound of any one of Enumerated Embodiments 25-35, wherein L is a bond, C₁₋₁₀alkylene, C(O)—C₁₋₁₀alkylene, C₁₋₆alkylene-C(O)—C₁₋₁₀alkylene, C₀₋₆alkylene-C(O)N(R^{y})—C₁₋₁₀alkylene, or L is L¹-L²-L³, wherein L¹ is a bond or C₁₋₆ alkylene, L² is a piperidinyl ring or piperazinyl ring, and L³ is C₁₋₁₀ alkylene.
Enumerated Embodiment 37. The compound of any one of Enumerated Embodiments 25-36, wherein Ring 2 is absent.
Enumerated Embodiment 38. The compound of any one of Enumerated Embodiments 25-36, wherein Ring 2 is 4- to 12-membered heterocyclyl optionally substituted with one or more R^{2a}.
Enumerated Embodiment 39. The compound of Enumerated Embodiment 38, wherein Ring 2 is

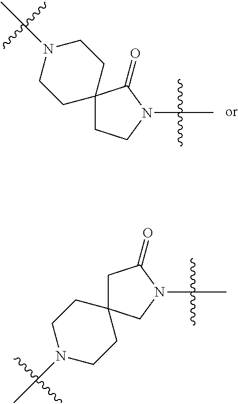 [SMILES: *N1CCC2(CC1)CCN(*)C2=O ; "or" ; *N1CCC2(CC1)CC(=O)N(*)C2]

optionally substituted with one or more R^{2a}.
Enumerated Embodiment 40. The compound of Enumerated Embodiment 1, wherein the compound is selected from the compounds provided in Table 1 and Table 2, a tautomer or stereoisomer thereof, or a pharmaceutically acceptable salt of any of the foregoing.
Enumerated Embodiment 41. The compound of Enumerated Embodiment 40, wherein the compound is selected from the compounds provided in Table 1, a tautomer or stereoisomer thereof, or a pharmaceutically acceptable salt of any of the foregoing.
Enumerated Embodiment 42. The compound of Enumerated Embodiment 40, wherein the compound is selected from the compounds provided in Table 2, a tautomer or stereoisomer thereof, or a pharmaceutically acceptable salt of any of the foregoing.
Enumerated Embodiment 43. A pharmaceutical composition, comprising a compound of any one of Enumerated Embodiments 1-42, a tautomer or stereoisomer thereof, or a pharmaceutically acceptable salt of any of the foregoing, and a pharmaceutically acceptable excipient.
Enumerated Embodiment 44. A method of treating a disease or disorder associated with PIP4K2C, comprising administering to a subject a compound of any of Enumerated Embodiments 1-42, or the pharmaceutical composition of Enumerated Embodiment 43.
Enumerated Embodiment 45. A method of treating a disease or disorder by modulating the level or activity of PIP4K2C, comprising administering to a subject a compound of any of Enumerated Embodiments 1-42, or a tautomer or stereoisomer thereof, or a pharmaceutically acceptable salt of any of the foregoing, or the pharmaceutical composition of Enumerated Embodiment 43.
Enumerated Embodiment 46. A method of treating a disease or disorder by modulating the activity of PIP4K2C, comprising administering to a subject a compound of any of Enumerated Embodiments 19-24 or 41, or a tautomer or stereoisomer thereof, or a pharmaceutically acceptable salt of any of the foregoing, or the pharmaceutical composition of Enumerated Embodiment 43.
Enumerated Embodiment 47. A method of treating a disease or disorder by modulating the level of PIP4K2C, comprising administering to a subject a compound of any of Enumerated Embodiments 25-39 or 42, or a tautomer or stereoisomer thereof, or a pharmaceutically acceptable salt of any of the foregoing, or the pharmaceutical composition of Enumerated Embodiment 43.
Enumerated Embodiment 48. The method of any one of Enumerated Embodiments 44-47, wherein the disease or disorder is a cancer, immune deficiency, autoimmune disease, infectious disease, or a combination thereof.
Enumerated Embodiment 49. The method of Enumerated Embodiment 48, wherein the cancer is bladder cancer, breast cancer, triple negative breast cancer, cervical cancer, colorectal carcinoma, colorectal carcinoma with MSI, colorectal carcinoma with MSS, gastric cancer, head and neck squamous cell carcinoma, hepatocellular carcinoma, leukemia, lung cancer, small cell lung cancer, non-small cell lung cancer, lymphoma, melanoma, Merkel cell carcinoma, multiple myeloma, pancreatic ductal carcinoma, or renal cell carcinoma.
Enumerated Embodiment 50. The method of Enumerated Embodiment 49, wherein the cancer is breast cancer, triple negative breast cancer, colorectal carcinoma, colorectal carcinoma with MSI, colorectal carcinoma with MSS, or non-small cell lung cancer.
Enumerated Embodiment 51. The method of any one of Enumerated Embodiments 44-47, wherein the disease or disorder is a neurodegenerative disease.
Enumerated Embodiment 52. A method of treating a viral infection by modulating the level of PIP4K2C, comprising administering to a subject a compound of any of Enumerated Embodiments 25-39 or 42, or a tautomer or stereoisomer thereof, or a pharmaceutically acceptable salt of any of the foregoing, or the pharmaceutical composition of Enumerated Embodiment 43.
Enumerated Embodiment 53. A method of preventing or treating necrotizing soft tissue infection (NSTI), comprising administering to a subject a compound of any of Enumerated Embodiments 25-39 or 42, or a tautomer or stereoisomer thereof, or a pharmaceutically acceptable salt of any of the foregoing, or the pharmaceutical composition of Enumerated Embodiment 43.
Enumerated Embodiment 54. A method of modulating the level or activity of at least one of PIP4K2A, PIP4K2B, and PIP4K2C, comprising administering to a subject a compound of any of Enumerated Embodiments 1-42, or a tautomer or stereoisomer thereof, or a pharmaceutically acceptable salt of any of the foregoing, or the pharmaceutical composition of Enumerated Embodiment 43.
Enumerated Embodiment 55. A method of modulating the activity of PIP4K2C, comprising administering to a subject a compound of any of Enumerated Embodiments 19-24 or 41, or a tautomer or stereoisomer thereof, or a pharmaceutically acceptable salt of any of the foregoing, or the pharmaceutical composition of Enumerated Embodiment 43.
Enumerated Embodiment 56. A method of treating a disease or disorder by modulating the level of PIP4K2C, comprising administering to a subject a compound of any of Enumerated Embodiments 25-39 or 42, or a tautomer or stereoisomer thereof, or a pharmaceutically acceptable salt of any of the foregoing, or the pharmaceutical composition of Enumerated Embodiment 43.
Enumerated Embodiment 57. A kit, comprising (i) a compound of any one of Enumerated Embodiments 1-42, or a stereoisomer or tautomer thereof, or a pharmaceutically acceptable salt of any of the foregoing, or the pharmaceutical composition of Enumerated Embodiment 43, and (ii) instructions for use in treating an PIP4K-mediated disease, disorder, or condition in an individual in need thereof.

### GENERAL SYNTHETIC METHODS AND INTERMEDIATES

The compounds of the present invention can be prepared by methods known to one of ordinary skill in the art and/or by reference to the schemes shown below and the synthetic examples that follow. Exemplary synthetic routes are set forth in Schemes 1, 1A, 2, 2A, 3, 3A, 4, 5, and 6, and in the Examples. One of ordinary skill in the art will recognize that numerous variations in reaction conditions including variations in solvent, reagents, catalysts, reaction temperatures and times are possible for each of the reactions described. Variation of order of synthetic steps and alternative synthetic routes are also possible.

In some embodiments, provided is a method of making a compound of Formula (I′) or (I), or any related formula such as (I-a), (I-b), (I-c), (I-d), (I-e), (I-f), (II), (II-a), (II-b), (II-c), (II-d), (II-e), (II-f), (II-g), (II-h), (III), (III-a), (III-b), (III-c), (III-d), or (III-e), or a tautomer or stereoisomer thereof, or a pharmaceutically acceptable salt of any of the foregoing. In some embodiments, the method is as shown in Scheme 1 or 1A. In some embodiments, the method comprises coupling a compound of formula

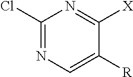

with a boronic acid of Intermediate 1,

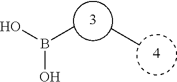

(Intermediate 1), to form Intermediate 2,

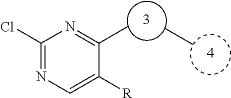

(Intermediate 2), wherein R, Ring 3, and Ring 4 are as defined for Formula (I′) or (I) and X is a halogen (e.g., chloro), wherein the reaction is optionally performed in the presence of one or more of a palladium catalyst (e.g., Pd(PPh₃)₄ or Pd(dppf)Cl₂), a base (e.g., Na₂CO₃ or KOAc), and/or a solvent (e.g., water, acetonitrile, dioxane, or a mixture thereof), wherein the reaction is optionally performed at elevated temperature (e.g., at about 80° C. or about 90° C.). In some embodiments, the compound of formula

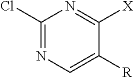

is 2,4,5-trichloropyrimidine. In some embodiments, the method further comprises coupling Intermediate 2 with Intermediate 3,

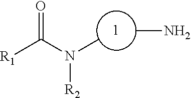

(Intermediate 3), to form a compound of Intermediate 4,

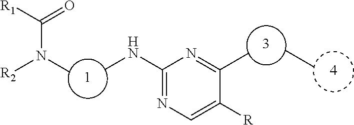

(Intermediate 4), wherein R, Ring 1, Ring 3, and Ring 4 are as defined for Formula (I′) or (I), and R¹ and R² may be taken together to form Ring 2, or C(O)R¹ is a protecting group and R² is hydrogen, C₁₋₄alkyl, or C₁₋₄haloalkyl, wherein the reaction is optionally performed in the presence of one or more of a palladium catalyst (e.g., Pd(OAc)₂), a ligand (e.g., Xantphos), a base (e.g., Cs₂CO₃), and/or a solvent (e.g., dioxane), wherein the reaction is optionally performed at elevated temperature (e.g., at about 100° C.). In some embodiments, C(O)R¹ is a protecting group and the method further comprises deprotecting Intermediate 4.

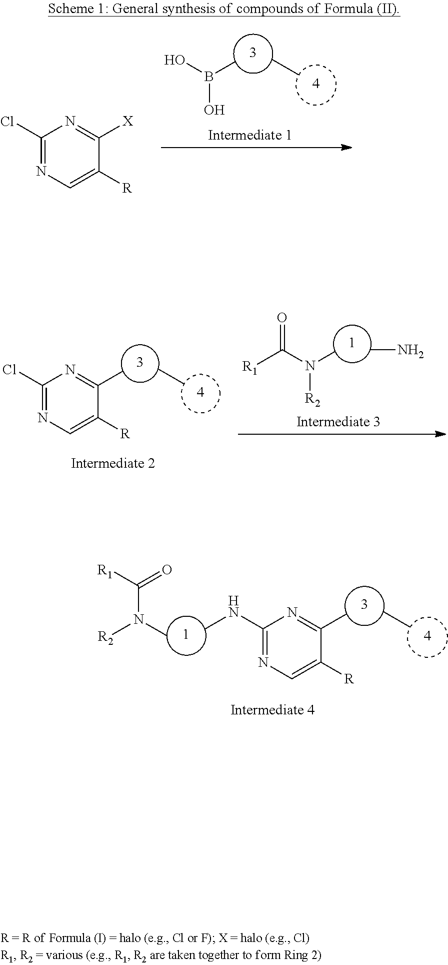

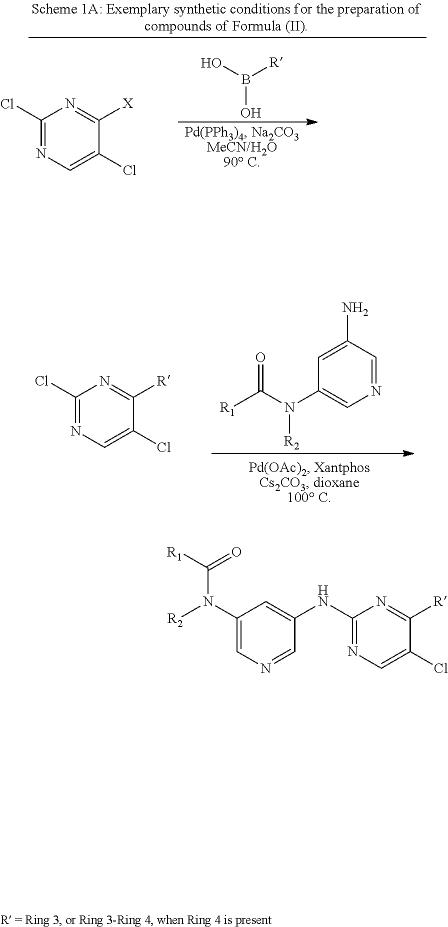

In some embodiments, provided is a method of making a compound of formula (III), or any related formula such as (III-a), (III-b), (III-c), (III-d), or (III-e), or a tautomer or stereoisomer thereof, or a pharmaceutically acceptable salt of any of the foregoing. In some embodiments, the method is as shown in Scheme 2, 2A, 3, 3A, 4, 5, or 6.

In some embodiments, the method is as shown in Scheme 2 or 2A. In some embodiments, the method comprises reacting a compound of Intermediate 5,

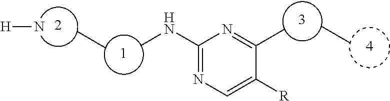

(Intermediate 5, which is an example of Intermediate 4, wherein R¹ and R² are taken together to form Ring 2), with a compound of Intermediate 6 (W″-Linker-U′) (wherein U′ is a functional group that upon reaction with the annular nitrogen of Ring 2 forms U; and W″ is a functional group that, upon reaction with another functional group W′, will form W) to form a compound of Intermediate 7,

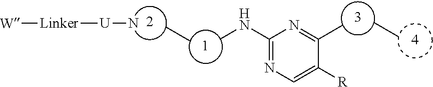

(Intermediate 7), wherein R, Linker, U, Ring 2, Ring 1 Ring 3, and Ring 4 are as defined in Formula (I′) or (I); wherein the reaction is optionally performed in the presence of one or more of a base (e.g., K₂CO₃) and a solvent (e.g., DMF), optionally at elevated temperature (e.g., 50° C.). In some embodiments, the method comprises reacting the compound of Intermediate 7 with a compound of Intermediate 8,

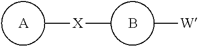

(Intermediate 8, wherein W′ is a functional group that, upon reaction with W″, forms W), to form a compound of Formula (III) wherein Ring 2 is present and Ring C is absent, wherein: Ring A, X, Ring B, and W are as defined in Formula (I′) or (I); wherein the reaction is optionally performed in the presence of one or more of a base (e.g., K₂CO₃) and a solvent (e.g., DMF), optionally at ambient temperature (e.g., 25° C.).

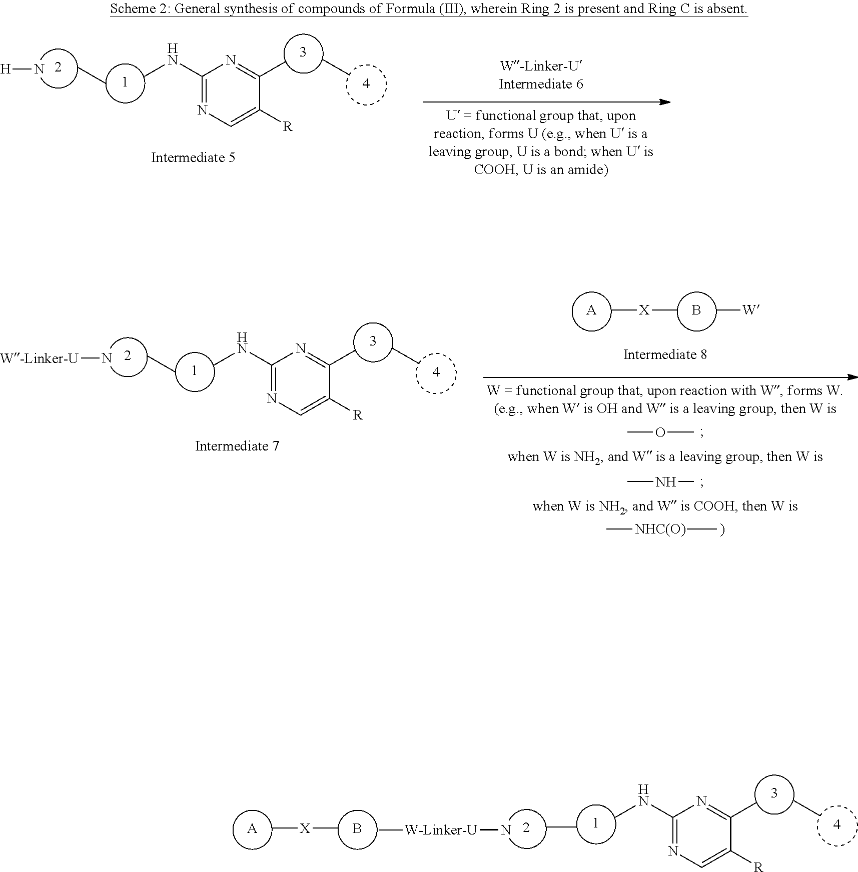

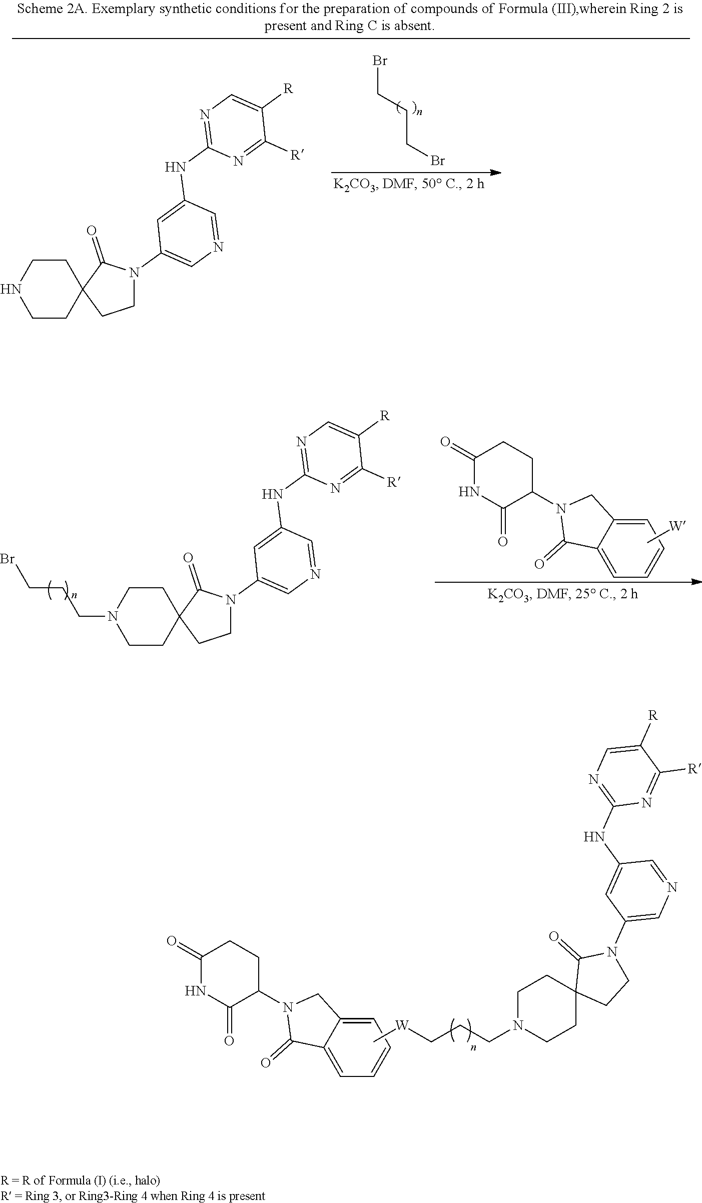

In some embodiments, provided is a method of making a compound of formula (III), or any related formula such as (III-a), (III-b), (III-c), (III-d), or (III-e), or a tautomer or stereoisomer thereof, or a pharmaceutically acceptable salt of any of the foregoing, as shown in Scheme 3 or 3A. In some embodiments, the method comprises reacting a compound of Intermediate 8 with a compound of Intermediate 6 to form a compound of Intermediate 9,

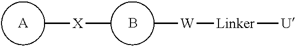

(Intermediate 9), wherein the reaction is optionally performed in the presence of one or more of a base (e.g., K₂CO₃) and a solvent (e.g., DMF), optionally at ambient temperature (e.g., 25° C.); wherein U′ is a functional group that, upon reaction with the annular nitrogen of Ring 2, forms U. In other embodiments, the method comprises reacting a compound of Intermediate 8 with a compound of Intermediate 6 to form a compound of Intermediate 9, wherein the reaction is optionally performed in the presence of one or more of a base (e.g., K₂CO₃) and a solvent (e.g., DMF), optionally at ambient temperature (e.g., 25° C.); wherein U′ is a protected functional group that, upon deprotection and reaction with the annular nitrogen of Ring 2, forms U. In some embodiments, the method comprises deprotecting U′ (e.g., when U′ is a carboxylic acid protected as a tert-butyl ester, the method comprises deprotecting the carboxylic acid, for instance, by reacting the carboxylic acid with an acid such as TFA in the presence of a solvent such as DCM). In some embodiments, the method comprising reacting the compound of Intermediate 9, or the deprotected compound of Intermediate 9), with Intermediate 5 to form a compound of Formula (III) wherein Ring 2 is present and Ring C is absent, wherein the reaction is optionally performed in the presence of one or more of an amide coupling reagent (e.g., N,N,N′,N′-tetramethylchloroformamidinium hexafluorophosphate (TCHF) and/or N-methyl imidazole (NMI)), a base (e.g., diisopropylethylamine, DIEA), and/or a solvent (e.g., DMF).

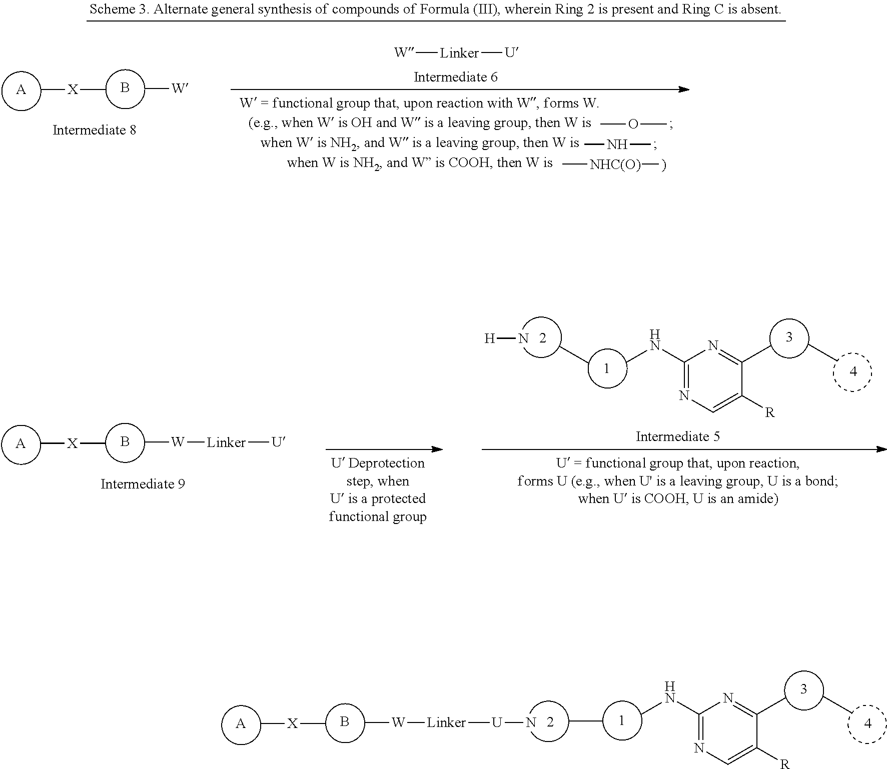

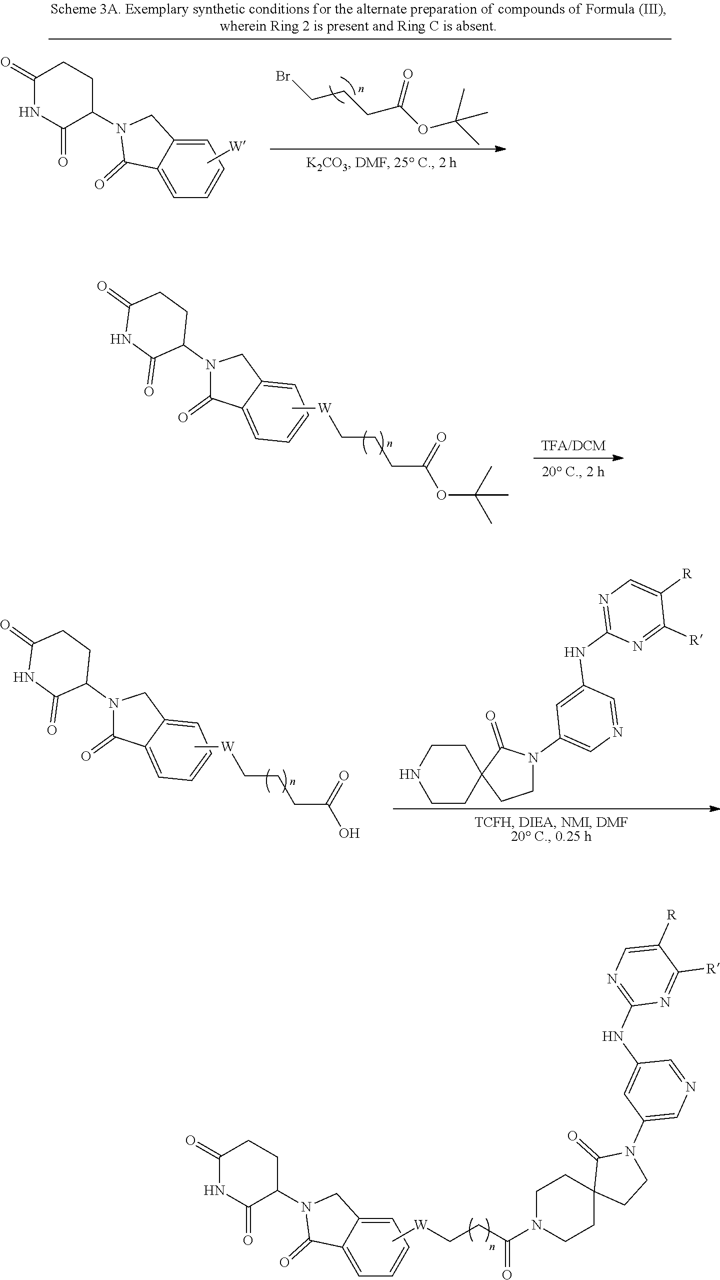

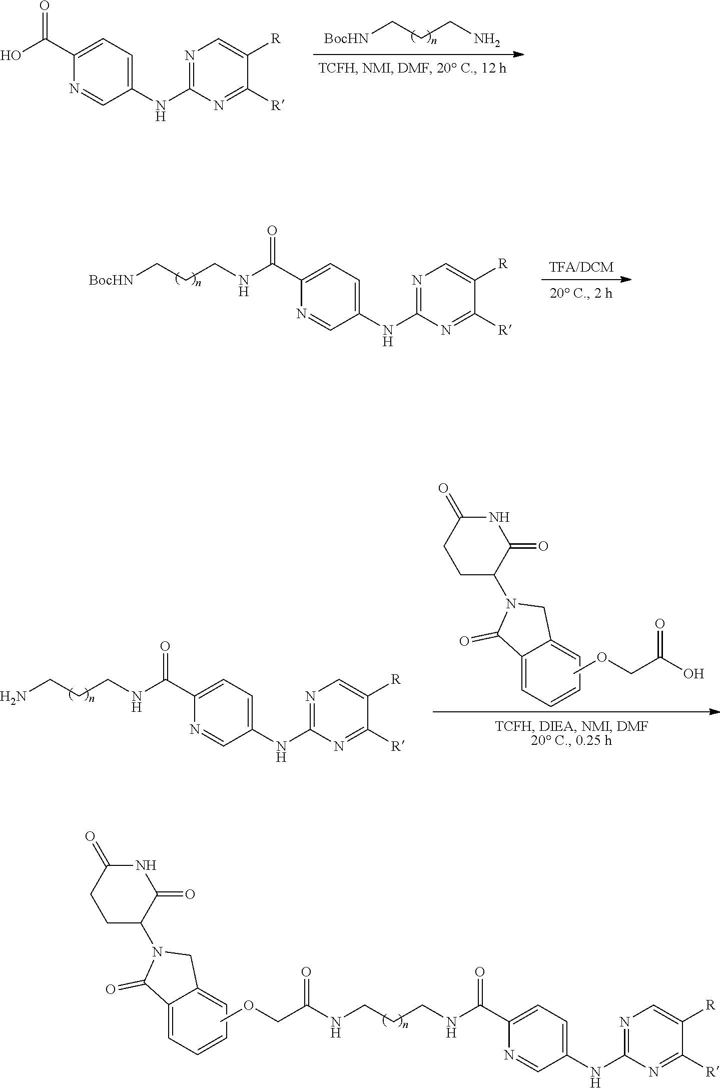

In some embodiments, provided is a method of making a compound of formula (III), or any related formula such as (III-a), (III-b), (III-c), (III-d), or (III-e), or a tautomer or stereoisomer thereof, or a pharmaceutically acceptable salt of any of the foregoing, as shown in Scheme 4. In some embodiments, the method comprises reacting a compound of Intermediate 5 with a compound of Intermediate 10 to form a compound of Intermediate 11,

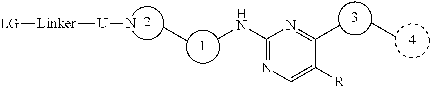

(Intermediate 11), wherein U′ is a functional group that, upon reaction with the annular nitrogen of Ring 2, forms U. In some embodiments, the reaction comprises reacting the compound of Intermediate 11 with the compound of Intermediate 12,

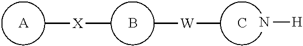

(Intermediate 12), to form a compound of Formula (III) wherein Ring 2 is present and Ring C is present, wherein LG is a leaving group.

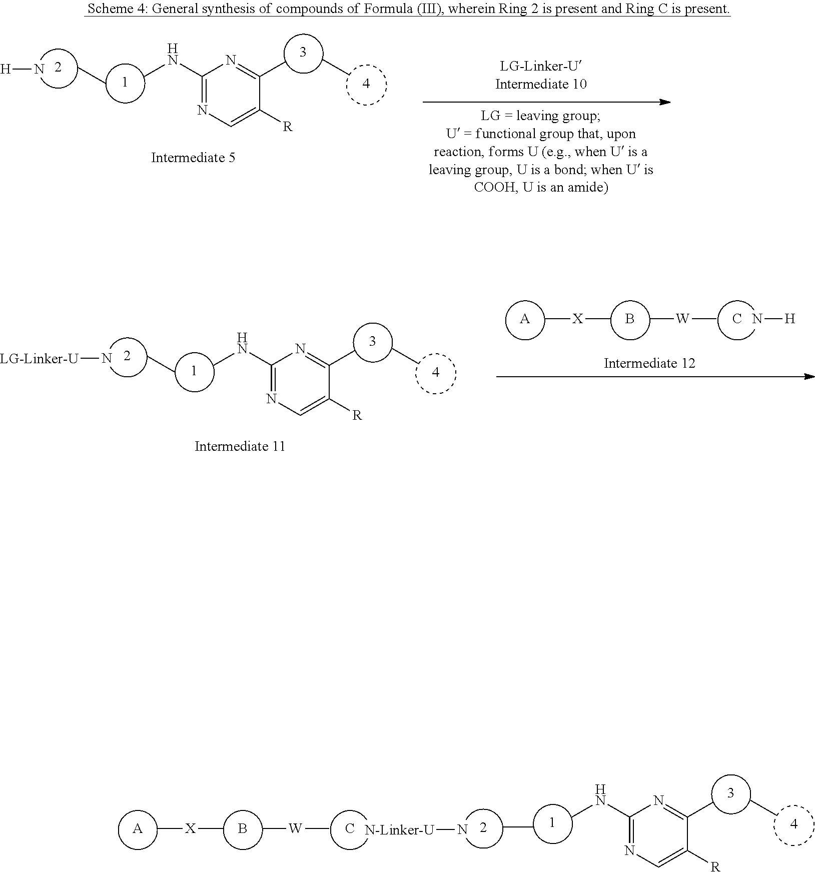

In some embodiments, provided is a method of making a compound of formula (III), or any related formula such as (III-a), (III-b), (III-c), (III-d), or (III-e), or a tautomer or stereoisomer thereof, or a pharmaceutically acceptable salt of any of the foregoing, as shown in Scheme 5. In some embodiments, the method comprises reacting a compound of Intermediate 13

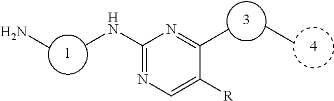

(Intermediate 13) with a compound of Intermediate 14 (W″-Linker-V′) to form a compound of Intermediate 15,

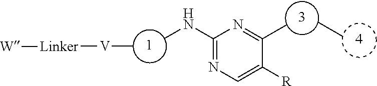

(Intermediate 15), wherein V′ is a functional group that, upon reaction with the primary amine of Intermediate 13, forms V. In some embodiments, the method comprises reacting a compound of Intermediate 15 with a compound of Intermediate 8 to form a compound of Formula (III), wherein Ring 2 is absent and Ring C is absent.

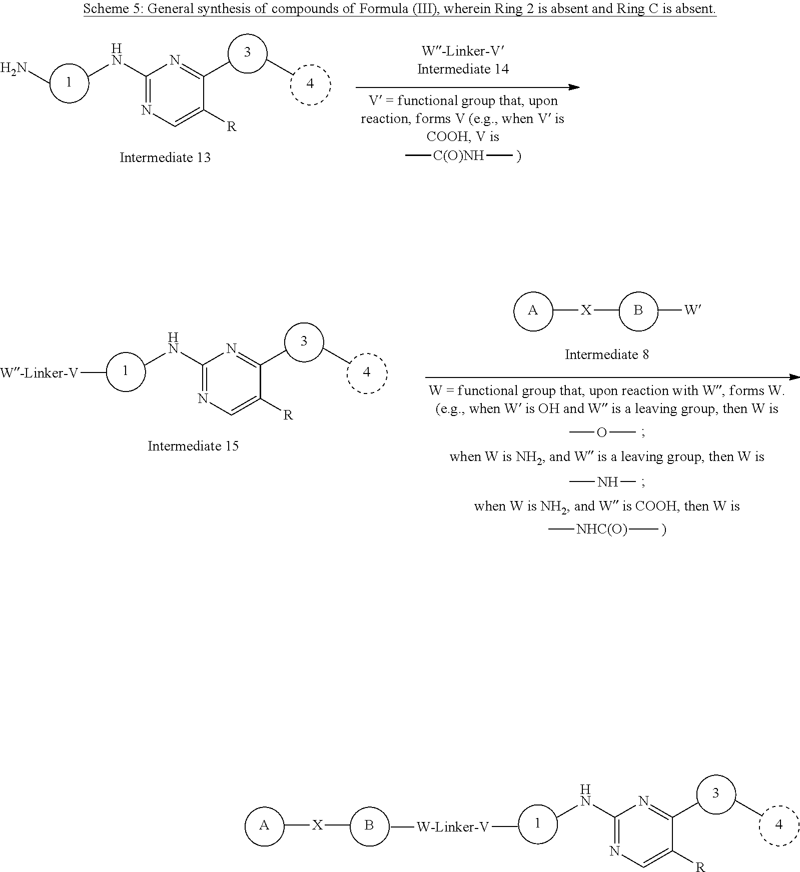

In some embodiments, provided is a method of making a compound of formula (III), or any related formula such as (III-a), (III-b), (III-c), (III-d), or (III-e), or a tautomer or stereoisomer thereof, or a pharmaceutically acceptable salt of any of the foregoing, as shown in Scheme 6. In some embodiments, the method comprises reacting a compound of Intermediate 13 with a compound of Intermediate 16 (LG-Linker-V′) to form a compound of Intermediate 17,

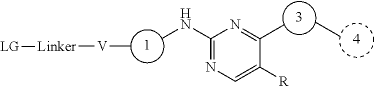

(Intermediate 17). In some embodiments, the method comprises reacting a compound of Intermediate 17 with a compound of Intermediate 12 to form a compound of Formula (III) wherein Ring 2 is absent and Ring C is present, wherein LG is a leaving group.

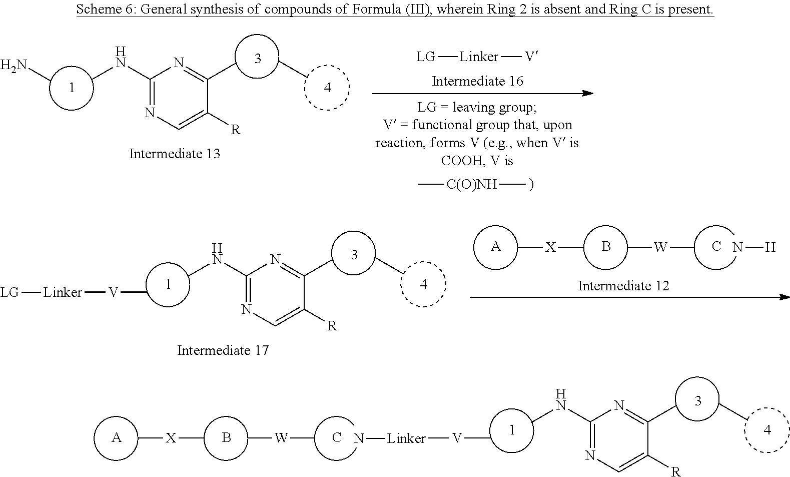

These and other aspects of the present invention will be further appreciated upon consideration of the following Examples, which are intended to illustrate certain particular embodiments of the invention but are not intended to limit its scope, as defined by the claims.

### EXAMPLES

### Synthetic Examples

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

TLC spots were visualized by UV light (254 and 220 nm). Purification by column and flash chromatography was carried out using silica gel (100-200 mesh). Solvent systems are reported as the ratio of solvents. NMR spectra were recorded on a Bruker 400 (400 MHz) spectrometer. ¹H chemical shifts are reported in 6 values in ppm with tetramethylsilane (TMS, =0.00 ppm) as the internal standard. LCMS spectra were obtained on an Agilent 1200 series 6110 or 6120 mass spectrometer with ESI (+) ionization mode. See, e.g., the data provided in Tables 3 and 4.

### Example I-1. Synthesis of tert-butyl 4-(2-(2,6-dioxopiperidin-3-yl)-1-oxoisoindolin-5-yl)piperidine-1-carboxylate (Intermediate A)

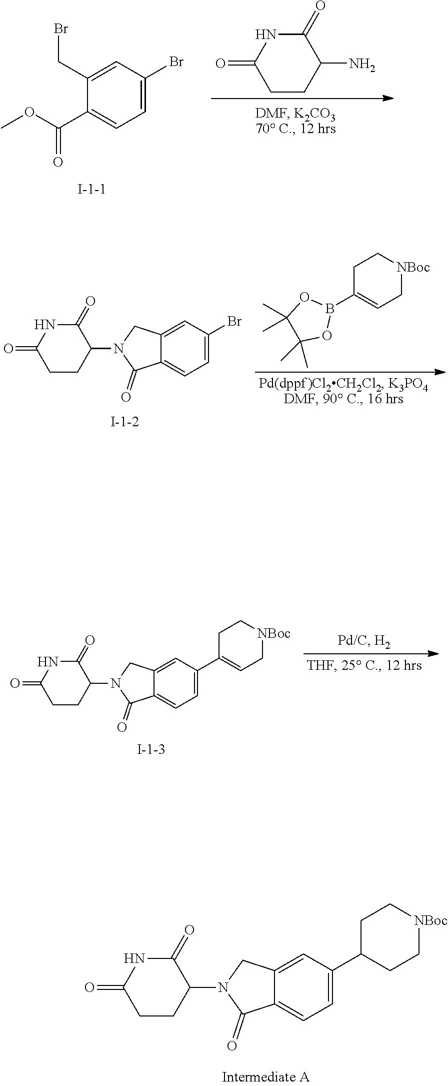

Synthesis of 3-(5-bromo-1-oxoisoindolin-2-yl)piperidine-2,6-dione (I-1-2). A mixture of methyl 4-bromo-2-(bromomethyl)benzoate (200 g, 649 mmol), 3-aminopiperidine-2,6-dione (128 g, 779 mmol, HCl), K₂CO₃ (273 g, 1948 mmol) in DMF (2500 mL) was degassed and purged with N₂ for 3 times, and then the mixture was stirred at 70° C. for 12 hrs under N₂ atmosphere. Crude LCMS indicated methyl 4-bromo-2-(bromomethyl)benzoate was consumed completely and the purity of product 2 was 81% (Rt=0.795 min, MS Calcd: 322.0; MS Found: 323.0 [M+H]⁺). The reaction mixture was poured into ice water (1.5 L) and stirred for another 30 mins. Lots of solid were precipitated. Then the suspension was filtered, and the filter cake was rinsed with EtOAc (500 mL×3), dried in vacuum to give the 3-(5-bromo-1-oxoisoindolin-2-yl)piperidine-2,6-dione (200 g, 95.30% yield) as a gray solid. LCMS (ES⁺): m/z 323.0 [M+H]⁺.

Synthesis of tert-butyl 4-(2-(2,6-dioxopiperidin-3-yl)-1-oxoisoindolin-5-yl)-3,6-dihydropyridine-1(2H)-carboxylate (I-1-3). A mixture of 3-(5-bromo-1-oxoisoindolin-2-yl)piperidine-2,6-dione (140 g, 433 mmol), tert-butyl 4-(4,4,5,5-tetramethyl-1,3,2-dioxaborolan-2-yl)-3,6-dihydro-2H-pyridine-1-carboxylate (174 g, 563 mmol), Pd(dppf)Cl₂·CH₂Cl₂ (17.69 g, 21.66 mmol), K₃PO₄ (110.36 g, 519 mmol) in DMF (1400 mL) was degassed and purged with N₂ for 3 times, and then the mixture was stirred at 90° C. for 16 hrs under N₂ atmosphere. Crude LCMS indicated 3-(5-bromo-1-oxoisoindolin-2-yl)piperidine-2,6-dione was consumed completely and the purity of product 3 was 49% (Rt=0.672 min, MS Calcd: 425.2; MS Found: 426.1 [M+H]⁺). The reaction mixture was diluted with H₂O 1000 mL and extracted with EA 3000 mL (1000 mL*3). The combined organic layers were washed with brine 3 L (1 L×3), dried over Na₂SO₄ filtered and concentrated under reduced pressure to give tert-butyl 4-(2-(2,6-dioxopiperidin-3-yl)-1-oxoisoindolin-5-yl)-3,6-dihydropyridine-1(2H)-carboxylate (160 g, 376.05 mmol) as a brown solid. LCMS (ES⁺): m/z 426.1 [M+H]⁺.

Synthesis of tert-butyl 4-(2-(2,6-dioxopiperidin-3-yl)-1-oxoisoindolin-5-yl)piperidine-1-carboxylate (Intermediate A). To a solution of tert-butyl 4-(2-(2,6-dioxopiperidin-3-yl)-1-oxoisoindolin-5-yl)-3,6-dihydropyridine-1(2H)-carboxylate (140 g, 329.04 mmol) in THF (1400 mL) was added Pd/C (10% purity, 140 g) under N₂ atmosphere. The suspension was degassed and purged with H₂ for 3 times. The mixture was stirred under H₂ (50 Psi) at 25° C. for 12 hr. Crude LCMS indicated tert-butyl 4-(2-(2,6-dioxopiperidin-3-yl)-1-oxoisoindolin-5-yl)-3,6-dihydropyridine-1(2H)-carboxylate was consumed completely and the purity of final product was 66% (Rt=0.865 mins, MS Calcd: 427.2; MS Found: 372.2 [M−56+H]⁺). The resultant mixture was filtered and the filter cake was rinsed with THE (200 mL×3). Then the combined filtrates were concentrated under reduced pressure to give the crude product. The crude was triturated with MTBE at 25° C. for 2 hrs. And then it was filtered and the filter cake was collected, dried in vacuum to afford the title product (Intermediate A) (50 g, 30.57% yield, 85% purity) as a gray solid. LCMS (ES⁺): m/z 372.2 [M+H−56]⁺. ¹H NMR (400 MHz, DMSO-d₆) δ ppm 1.42 (s, 9H) 1.47-1.62 (m, 2H) 1.78 (br d, J=12.01 Hz, 2H) 1.92-2.06 (m, 1H) 2.39 (br dd, J=13.2, 4.4 Hz, 1H) 2.60 (br d, J=17.2 Hz, 1H) 2.74-2.98 (m, 4H) 4.09 (br d, J=11.2 Hz, 2H) 4.23-4.50 (m, 2H) 5.10 (br dd, J=13.2, 5.2 Hz, 1H) 7.40 (d, J=7.6 Hz, 1H) 7.49 (s, 1H) 7.60-7.71 (m, 1H) 10.98 (s, 1H).

### Example I-2. Synthesis of tert-butyl 4-((2-(2,6-dioxopiperidin-3-yl)-1-oxoisoindolin-5-yl)oxy)piperidine-1-carboxylate (Intermediate B)

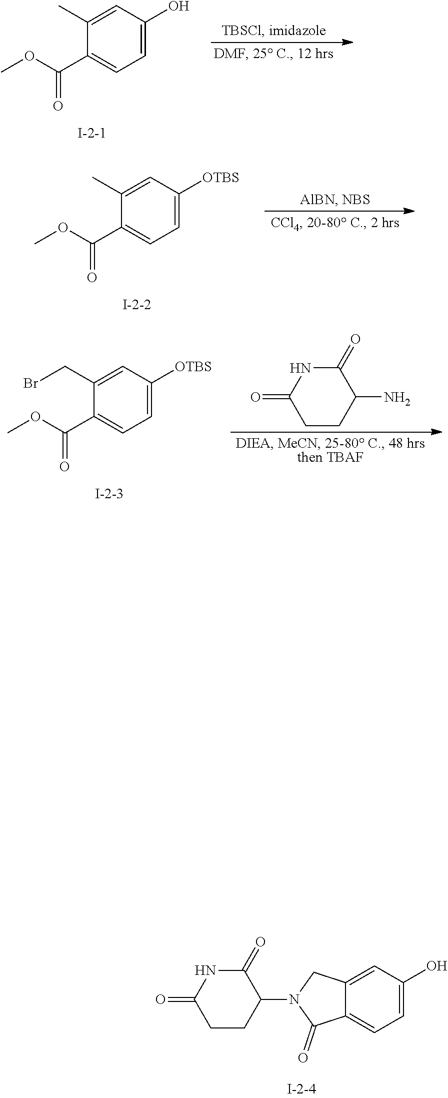

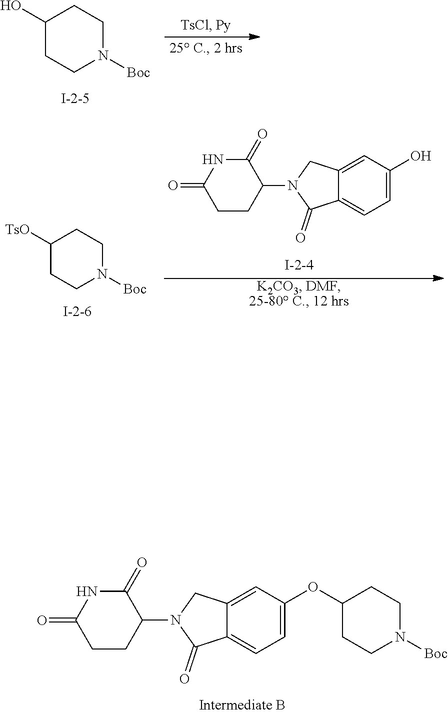

Synthesis of methyl 4-((tert-butyldimethylsilyl)oxy)-2-methylbenzoate (I-2-2). To a mixture of methyl 4-hydroxy-2-methylbenzoate (170 g, 1.02 μmol, 1.00 eq) in DMF (2000 mL) were added imidazole (209 g, 3.07 μmol, 3.00 eq) and TBSCl (231 g, 1.53 μmol, 188 mL, 1.50 eq) at 25° C. The reaction mixture was stirred at 25° C. for 12 hrs. Crude LCMS indicated methyl 4-hydroxy-2-methylbenzoate was consumed completely and the purity of methyl 4-((tert-butyldimethylsilyl)oxy)-2-methylbenzoate was 29.4% (Rt=1.011 min, MS Calcd: 280.1, MS found: 281.1 [M+H]⁺). H₂O (3000 mL) was added to the mixture, it was extracted with ethyl acetate (1500 mL*3). The combined organic layers were washed with brine (2000 mL), dried over Na₂SO₄, filtered and concentrated under reduced pressure to give methyl 4-((tert-butyldimethylsilyl)oxy)-2-methylbenzoate (360 g, crude) as a green solid. LCMS (ES⁺): m/z 281.1 [M+H]⁺.

Synthesis of methyl 2-(bromomethyl)-4-((tert-butyldimethylsilyl)oxy)benzoate (I-2-3). To a solution of methyl 4-((tert-butyldimethylsilyl)oxy)-2-methylbenzoate (200 g, 713 mmol, 1.00 eq) in CCl₄ (2000 mL) were added NBS (150 g, 843 mmol, 1.18 eq) and AIBN (30.00 g, 183 mmol, 2.56e-1.00 eq) at 20° C. The mixture was stirred at 80° C. for 2 h. Crude LCMS indicated methyl 4-((tert-butyldimethylsilyl)oxy)-2-methylbenzoate was consumed completely and the purity of methyl 2-(bromomethyl)-4-((tert-butyldimethylsilyl)oxy)benzoate was 58.7% (Rt=0.996 min, MS Calcd: 358.1, MS found: 359.0 [M+H]⁺). The mixture was filtered and the filtrate was concentrated under reduced pressure. The residue was purified by MPLC (SiO₂, Petroleum ether/Ethyl acetate=1:0 to 10:1) to give methyl 2-(bromomethyl)-4-((tert-butyldimethylsilyl)oxy)benzoate (275 g, crude) as a yellow solid. LCMS (ES⁺): m/z 359.1 [M+H]⁺.

Synthesis of 3-(5-hydroxy-1-oxoisoindolin-2-yl)piperidine-2,6-dione (I-2-4). To a solution of methyl 2-(bromomethyl)-4-((tert-butyldimethylsilyl)oxy)benzoate (220 g, 612 mmol, 1.00 eq) and 3-aminopiperidine-2,6-dione; hydrochloride (111 g, 673 mmol, 1.10 eq) in MeCN (1500 mL) was added DIEA (237 g, 1.84 μmol, 320 mL, 3.00 eq) at 25° C. The mixture was stirred at 80° C. for 48 h under N₂. TLC (SiO₂, Petroleum ether/Ethyl acetate=3:1) indicated methyl 2-(bromomethyl)-4-((tert-butyldimethylsilyl)oxy)benzoate was consumed completely and one new spot formed. Then tetrabutylammonium; fluoride; trihydrate (612 mL, 1.00 M in THF, 1.00 eq) was added to the mixture. The mixture was concentrated under reduced pressure. The residue was purified by MPLC (SiO₂, Petroleum ether/Ethyl acetate=1:0 to 0:1). The crude product was triturated with EtOAc (1500 mL) at 0° C. for 10 min to give 3-(5-hydroxy-1-oxoisoindolin-2-yl)piperidine-2,6-dione (120 g, 461.10 mmol, 75.31% yield) as a blue solid.

Synthesis of tert-butyl 4-(tosyloxy)piperidine-1-carboxylate (I-2-6). To a solution of tert-butyl 4-hydroxypiperidine-1-carboxylate (100 g, 497 mmol, 1.00 eq) in Py (2500 mL) was added 4-methylbenzenesulfonyl chloride (171 g, 894 mmol, 1.80 eq) at 25° C. The mixture was stirred at 25° C. for 2 h. Crude LCMS indicated the purity of Compound 2A was 26.7% (Rt=0.562 min, MS Calcd: 355.1; MS Found: 300.1 [M−56+H]⁺). H₂O (2000 mL) was added to the mixture. The mixture was filtered and the filter cake was dried under reduced pressure to give tert-butyl 4-(tosyloxy)piperidine-1-carboxylate (800 g, crude) as a white solid. LCMS (ES⁺): m/z 378.1 [M+Na]⁺.

Synthesis of tert-butyl 4-((2-(2,6-dioxopiperidin-3-yl)-1-oxoisoindolin-5-yl)oxy)piperidine-1-carboxylate (Intermediate B). To a mixture of 3-(5-hydroxy-1-oxoisoindolin-2-yl)piperidine-2,6-dione (20.0 g, 76.9 mmol, 1.00 eq) and tert-butyl 4-(tosyloxy)piperidine-1-carboxylate (95.6 g, 269 mmol, 3.50 eq) in DMF (1000 mL) was added K₂CO₃ (31.9 g, 230 mmol, 3.00 eq) at 25° C. under N₂ atmosphere. The reaction mixture was stirred at 80° C. for 12 h. Crude LCMS indicated 3-(5-hydroxy-1-oxoisoindolin-2-yl)piperidine-2,6-dione was consumed completely and the purity of Int. B was 9.3% (Rt=0.430 min, MS Calcd: 443.2; MS Found: 388.3 [M−56+H]⁺). H₂O (5000 mL) and EtOAc (500 mL) were added to the mixture, the mixture was filtered and the filter cake was dried under reduced pressure. The crude product was triturated with EtOAc (100 mL) at 0° C. for 10 min to give tert-butyl 4-((2-(2,6-dioxopiperidin-3-yl)-1-oxoisoindolin-5-yl)oxy)piperidine-1-carboxylate (Intermediate B) (22 g, 48.41 mmol, 62.99% yield, 97.58% purity) as a white solid. LCMS (ES⁺): m/z 388.2 [M+H−56]⁺. ¹H NMR (400 MHz, DMSO-d6) δ=10.98 (s, 1H), 7.63 (d, J=8.4 Hz, 1H), 7.23 (s, 1H), 7.10 (br d, J=8.3 Hz, 1H), 5.08 (br dd, J=13.3, 5.0 Hz, 1H), 4.70 (br s, 1H), 4.39 (br d, J=17.3 Hz, 1H), 4.27 (br d, J=17.1 Hz, 1H), 3.77-3.59 (m, 2H), 3.28-3.12 (m, 2H), 2.98-2.84 (m, 1H), 2.64-2.56 (m, 1H), 2.49-2.29 (m, 1H), 2.10-1.83 (m, 3H), 1.56 (br d, J=9.4 Hz, 2H), 1.42 (s, 9H).

### Example I-3. Synthesis of tert-butyl 4-(4-((2,6-dioxopiperidin-3-yl)amino)phenyl)piperidine-1-carboxylate (Intermediate C)

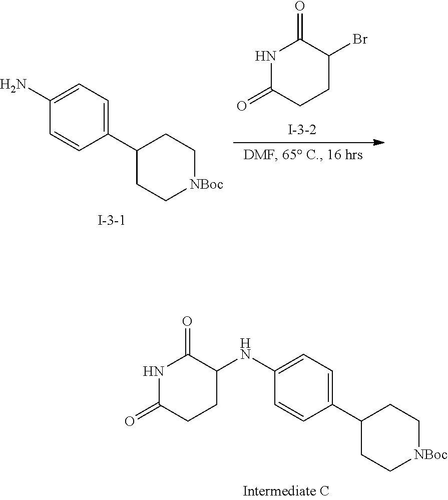 [SMILES: CC(C)(C)OC(=O)N1CCC(c2ccc(N)cc2)CC1 ; O=C1CCC(Br)C(=O)N1 ; CC(C)(C)OC(=O)N1CCC(c2ccc(NC3CCC(=O)NC3=O)cc2)CC1]

A mixture of tert-butyl 4-(4-aminophenyl)piperidine-1-carboxylate (104.22 g, 542 mmol), 3-bromopiperidine-2,6-dione (100 g, 361 mmol), NaHCO₃ (91.18 g, 1085 mmol) in DMF (1000 mL) was degassed and purged with N₂ for 3 times. Then the mixture was stirred at 65° C. for 16 hours under N₂ atmosphere. TLC indicated tert-butyl 4-(4-aminophenyl)piperidine-1-carboxylate was consumed completely and one new spot was observed. The reaction mixture was slowly poured into ice water (3 L) with stirring during 10 mins. Some solid were collected by filtration, rinsed with EtOAc (300 mL×3) and dried in vacuum to give desired tert-butyl 4-(4-((2,6-dioxopiperidin-3-yl)amino)phenyl)piperidine-1-carboxylate (Intermediate C) (101 g, 68.8% yield, 96.5% purity) as a white solid. LCMS (ES⁺): m/z 332.1 [M+H−56]⁺.

### Example I-4. Synthesis of tert-butyl 4-(1-(2,6-dioxopiperidin-3-yl)-3-methyl-2-oxo-2,3-dihydro-1H-benzo[d]imidazol-5-yl)piperidine-1-carboxylate (Intermediate D)

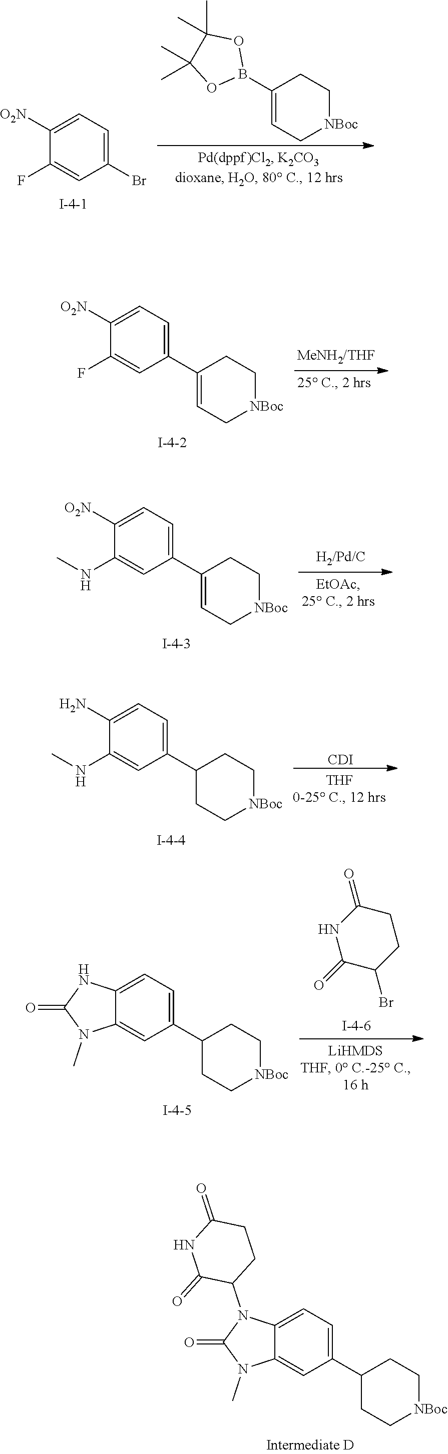

Synthesis of tert-butyl 4-(3-fluoro-4-nitrophenyl)-3,6-dihydropyridine-1(2H)-carboxylate (I-4-2). A mixture of 4-bromo-2-fluoro-1-nitrobenzene (355 g, 1617 mmol), tert-butyl 4-(4,4,5,5-tetramethyl-1,3,2-dioxaborolan-2-yl)-3, 6-dihydro-2H-pyridine-1-carboxylate (500 g, 1617 mmol), Pd(dppf)Cl₂ (47.32 g, 64.7 mmol), K₂CO₃ (167 g, 1.21 mol) in dioxane (5000 mL) and H₂O (500 mL) was degassed and purged with N₂ for 3 times, and then the mixture was stirred at 80° C. for 12 hrs under N₂ atmosphere. Crude LCMS indicated 4-bromo-2-fluoro-1-nitrobenzene was consumed completely and the purity of tert-butyl 4-(3-fluoro-4-nitrophenyl)-3,6-dihydropyridine-1(2H)-carboxylate was 92% (overlapped with catalyst peak; Rt=0.824 min, MS Calcd: 322.3, MS found: 223.0 (M−100+1). The reaction mixture was diluted with H₂O 4000 mL and extracted with ethyl acetate 15 L (5 L*3). The combined organic layers were washed with brine 9 L (3 L*3), dried over Na₂SO₄, filtered and concentrated under reduced pressure to give tert-butyl 4-(3-fluoro-4-nitrophenyl)-3,6-dihydropyridine-1(2H)-carboxylate (500 g, crude) as a brown solid. LCMS (ES⁺): m/z 223.0 [M+H−100]⁺.

Synthesis of tert-butyl 4-(3-(methylamino)-4-nitrophenyl)-3,6-dihydropyridine-1(2H)-carboxylate (I-4-3). Two batches of this reaction were conducted in parallel. To a solution of tert-butyl 4-(3-fluoro-4-nitrophenyl)-3,6-dihydropyridine-1(2H)-carboxylate (247 g, 766 mmol) in THF (1000 mL) was added MeNH₂ (2 M, 988 mL). The mixture was stirred at 20° C. for 2 hours. Crude LCMS indicated tert-butyl 4-(3-fluoro-4-nitrophenyl)-3,6-dihydropyridine-1(2H)-carboxylate was consumed completely and the purity of tert-butyl 4-(3-(methylamino)-4-nitrophenyl)-3,6-dihydropyridine-1(2H)-carboxylate was 56% (Rt=1.058 mins, MS Calcd: 333.2, MS found: 334.2 (M+1). The reaction mixture was quenched by addition NH₄Cl 2000 mL at 0° C., and then diluted with NaHCO₃2000 mL and extracted with EtOAc 6000 mL (2000 mL*3). The combined organic layers were washed with brine 6000 mL (2000 mL*3), dried over Na₂SO₄, filtered and concentrated under reduced pressure to give tert-butyl 4-(3-(methylamino)-4-nitrophenyl)-3,6-dihydropyridine-1(2H)-carboxylate (420 g, 1.26 μmol, 82.20% yield) as a yellow solid. The crude was used for next step without further purification. LCMS (ES⁺): m/z 334.2 [M+H]⁺. ¹H NMR (400 MHz, DMSO-d6) δ ppm 8.23 (br d, J=4.8 Hz, 1H) 7.96-8.04 (m, 1H) 6.76-6.87 (m, 2H) 6.39 (br s, 1H) 4.00-4.04 (m, 2H) 3.54 (br t, J=5.6 Hz, 2H) 2.99 (d, J=5.2 Hz, 3H) 2.51-2.53 (m, 1H) 2.44-2.49 (m, 1H) 1.42 (s, 9H).

Synthesis of tert-butyl 4-(4-amino-3-(methylamino)phenyl)piperidine-1-carboxylate (I-4-4). Three batches of this reaction were conducted in parallel. To a solution of tert-butyl 4-(3-(methylamino)-4-nitrophenyl)-3,6-dihydropyridine-1(2H)-carboxylate (140 g, 419.94 mmol, 1 eq) in EtOAc (2000 mL) was added Pd/C (10%, 15 g) under N₂ atmosphere. The suspension was degassed and purged with H₂ for 3 times. The mixture was stirred under H₂ (50 Psi) at 25° C. for 12 hrs. Crude LCMS indicated tert-butyl 4-(3-(methylamino)-4-nitrophenyl)-3,6-dihydropyridine-1(2H)-carboxylate was consumed completely and the purity of tert-butyl 4-(4-amino-3-(methylamino)phenyl)piperidine-1-carboxylate was 96% (Rt=0.952 min, MS Calcd: 305.2, MS found: 250.2 (M−56+1). The mixture was filtered through a Celite pad, and the filtrate was dried in vacuum to give a residue. The residue was triturated with MTBE (500 mL) at 25° C. for 60 mins. The solid was collected by filtration, rinsed with MTBE (500 mL×3) and dried in vacuum to give tert-butyl 4-(4-amino-3-(methylamino)phenyl)piperidine-1-carboxylate (310 g, 78.63% yield, 97.6% purity) as a purple solid. LCMS (ES⁺): m/z 250.2 [M+H−56]⁺. ¹H NMR (400 MHz, DMSO-d6) δ ppm 6.44 (d, J=7.6 Hz, 1H) 6.26 (dd, J=7.6, 1.6 Hz, 1H) 6.23 (s, 1H) 4.51 (q, J=4.8 Hz, 1H) 4.26 (s, 2H) 3.98-4.12 (m, 2H) 2.74 (br s, 2H) 2.69 (d, J=5.2 Hz, 3H) 2.39-2.48 (m, 1H) 1.68 (br d, J=12.4 Hz, 2H) 1.41 (s, 10H).

Synthesis of tert-butyl 4-(3-methyl-2-oxo-2,3-dihydro-1H-benzo[d]imidazol-5-yl)piperidine-1-carboxylate (I-4-5). Three batches of this reaction were conducted in parallel. To a solution of tert-butyl 4-(4-amino-3-(methylamino)phenyl)piperidine-1-carboxylate (103 g, 337 mmol) in THF (1500 mL) was added CDI (57.4 g, 354 mmol) at 0° C. under N₂. The mixture was stirred at 25° C. for 12 hrs. Crude LCMS indicated tert-butyl 4-(4-amino-3-(methylamino)phenyl)piperidine-1-carboxylate was consumed completely and the purity of tert-butyl 4-(3-methyl-2-oxo-2,3-dihydro-1H-benzo[d]imidazol-5-yl)piperidine-1-carboxylate was 98% (Rt=0.554 min, MS Calcd: 331.1, MS found: 332.3 (M+1). The reaction mixture was concentrated under reduced pressure to give residue. The residue was triturated with MTBE (1000 mL) at 25° C. for 2 hrs. The solid was collected by filtration, rinsed with MTBE 1800 mL (600 mL×3) and dried in vacuum to give tert-butyl 4-(3-methyl-2-oxo-2,3-dihydro-1H-benzo[d]imidazol-5-yl)piperidine-1-carboxylate (260 g, 77.54% yield) as a white solid. LCMS (ES⁺): m/z 332.3 [M+H]⁺. ¹H NMR (400 MHz, DMSO-d6) δ ppm 10.69 (s, 1H) 6.99 (s, 1H) 6.88 (s, 1H) 6.86 (s, 1H) 6.82-6.85 (m, 1H) 4.08 (br d, J=11.2 Hz, 2H) 3.25 (s, 3H) 2.72-3.03 (m, 2H) 2.67 (tt, J=12.0, 3.2 Hz, 1H) 1.74 (br d, J=13.2 Hz, 2H) 1.52 (qd, J=12.4, 4.4 Hz, 2H) 1.41 (s, 9H).

Synthesis of tert-butyl 4-(1-(2,6-dioxopiperidin-3-yl)-3-methyl-2-oxo-2,3-dihydro-1H-benzo[d]imidazol-5-yl)piperidine-1-carboxylate (Intermediate D). Five batches of this reaction were conducted in parallel. To a solution of tert-butyl 4-(3-methyl-2-oxo-2,3-dihydro-1H-benzo[d]imidazol-5-yl)piperidine-1-carboxylate (50 g, 150 mmol) in THF (1000 mL) was dropwise added LiHMDS (1 M, 452 mL) at 0° C. under N₂. After addition, the mixture was warmed to 25° C. slowly and stirred for another 1 hr. Then 3-bromopiperidine-2,6-dione (49.3 g, 256 mmol, 1.7 eq) was added at this temperature under N₂. The reaction mixture was stirred at 25° C. for another 12 hrs. TLC indicated tert-butyl 4-(3-methyl-2-oxo-2,3-dihydro-1H-benzo[d]imidazol-5-yl)piperidine-1-carboxylate was consumed completely and one new spot was observed. The reaction mixture was quenched by addition sat.aq.NH₄Cl 500 mL at 0° C., and then diluted with H₂O 500 mL and extracted with EtOAc 900 mL (300 mL*3). The combined organic layers were washed with brine 900 mL (300 mL*3), dried over Na₂SO₄ filtered and concentrated under reduced pressure to give a residue. The residue was purified by column chromatography (SiO₂, dichloromethane/Ethyl acetate=1/0 to 1/1) to give tert-butyl 4-(1-(2,6-dioxopiperidin-3-yl)-3-methyl-2-oxo-2,3-dihydro-1H-benzo[d]imidazol-5-yl)piperidine-1-carboxylate (Intermediate D) (100 g, 74.89% yield) as a white solid. LCMS (ES⁺): m/z 387.3 [M+H−56]⁺. ¹H NMR (400 MHz, DMSO-d6) δ ppm 11.08 (s, 1H) 7.10 (d, J=1.2 Hz, 1H) 6.98-7.04 (m, 1H) 6.91 (dd, J=8.4, 1.2 Hz, 1H) 5.33 (dd, J=12.8, 5.4 Hz, 1H) 4.02-4.14 (m, 2H) 3.33 (s, 3H) 2.58-2.93 (m, 6H) 1.94-2.05 (m, 1H) 1.86-1.93 (m, 1H) 1.75 (br d, J=12.4 Hz, 2H) 1.49-1.63 (m, 2H) 1.42 (s, 9H) 1.21-1.27 (m, 1H) 1.17 (t, J=7.2 Hz, 1H).

### Example 1. Synthesis of 2-(5-((4-([1,1′-biphenyl]-3-yl)-5-chloropyrimidin-2-yl)amino)pyridin-3-yl)-2,8-diazaspiro[4.5]decan-1-one (Compound 1)

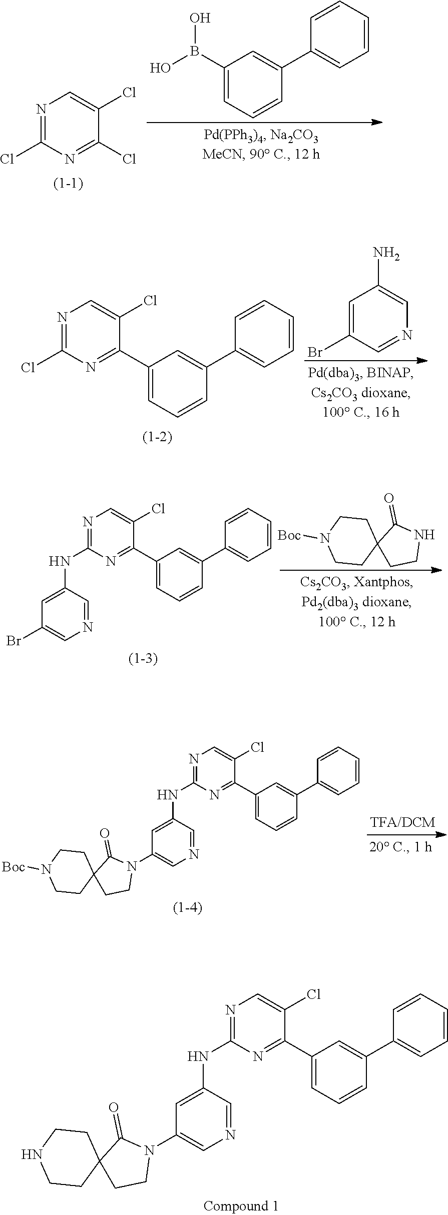

Synthesis of 4-([1,1′-biphenyl]-3-yl)-2,5-dichloropyrimidine (1-1). To a solution of (3-phenylphenyl)boronic acid (100 g, 505 mmol, 1.00 eq) and 2,4,5-trichloropyrimidine (1-1) (92.6 g, 505 mmol, 1.00 eq) in MeCN (1500 mL) were added Na₂CO₃ (107 g, 1.01 μmol, 2.00 eq) and Pd(PPh₃)₄ (5.84 g, 5.05 mmol, 0.0100 eq) at 25° C. The resulting mixture was stirred at 90° C. for 12 h under N₂. LCMS showed the desired mass was detected. The mixture was filtered, and the filtrate was concentrated under reduced pressure to give a residue. The crude product was triturated with MeOH (2 L) and H₂O (4 L) at 15° C. for 0.5 h and then filtered. The filter cake was dried under reduced pressure to give the title compound (1-2) (95.0 g, 315 mmol, 31.2% yield) as a white solid. LCMS (ES⁺): m/z 301.0 [M+H]⁺.

Synthesis of 4-([1,1′-biphenyl]-3-yl)-N-(5-bromopyridin-3-yl)-5-chloropyrimidin-2-amine (1-3). To a solution of 2,5-dichloro-4-(3-phenylphenyl)pyrimidine (1-1) (1.00 g, 3.32 mmol, 1.00 eq) in dioxane (10 mL) were added 5-bromopyridin-3-amine (632 mg, 3.65 mmol, 1.10 eq), BINAP (124 mg, 199 μmol, 0.0600 eq), Cs₂CO₃ (2.16 g, 6.64 mmol, 2.00 eq) and Pd₂(dba)₃ (122 mg, 133 μmol, 0.0400 eq) at 20° C. under N₂ atmosphere. The mixture was stirred at 100° C. for 12 h. LCMS showed ˜22% of 5-bromopyridin-3-amine and ˜20% of 2,5-dichloro-4-(3-phenylphenyl)pyrimidine were remaining (1-1), and ˜23% of the desired mass was detected. Then BINAP (62.0 mg), Pd₂(dba)₃ (61.0 mg) and Cs₂CO₃ (1.08 g) were added to the mixture at 20° C. The mixture was stirred at 100° C. for 4 h. LCMS showed ˜16% of 5-bromopyridin-3-amine was remaining, and ˜29% of the desired mass was detected. The reaction mixture was concentrated under reduced pressure to remove solvent. The residue was purified by column chromatography (SiO₂, Petroleum ether:Ethyl acetate=10:1 to 25:1) to give the title compound (1-3) (370 mg, 845 μmol, 25.5% yield) as a yellow solid. LCMS (ES⁺): m/z 437.0 [M+H]⁺.

Synthesis of tert-butyl 2-(5-((4-([1,1′-biphenyl]-3-yl)-5-chloropyrimidin-2-yl)amino)pyridin-3-yl)-1-oxo-2,8-diazaspiro[4.5]decane-8-carboxylate (1-4). To a solution of 4-([1,1′-biphenyl]-3-yl)-N-(5-bromopyridin-3-yl)-5-chloropyrimidin-2-amine (1-3) (240 mg, 548 μmol, 1.00 eq) in dioxane (7 mL) were added tert-butyl 1-oxo-2,8-diazaspiro[4.5]decane-8-carboxylate (69.7 mg, 274 μmol, 0.500 eq), Pd₂(dba)₃ (12.6 mg, 13.7 μmol, 0.0250 eq), Cs₂CO₃ (179 mg, 548 μmol, 1.00 eq) and (5-diphenylphosphanyl-9,9-dimethyl-xanthen-4-yl)-diphenyl-phosphane (23.8 mg, 41.1 μmol, 0.0750 eq) at 20° C. under N₂ atmosphere. The mixture was stirred at 100° C. for 12 h. LCMS showed 4-([1,1′-biphenyl]-3-yl)-N-(5-bromopyridin-3-yl)-5-chloropyrimidin-2-amine (1-3) was consumed completely and the desired mass was detected. The reaction mixture was concentrated under reduced pressure to remove solvent. The residue was purified by prep-TLC (SiO₂, Petroleum ether:Ethyl acetate=3:1) to give the title compound (1-4) (190 mg, 311 μmol, 56.7% yield) as a brown solid. LCMS (ES⁺): m/z 611.4 [M+H]⁺.

Synthesis of 2-(5-((4-([1,1′-biphenyl]-3-yl)-5-chloropyrimidin-2-yl)amino)pyridin-3-yl)-2,8-diazaspiro[4.5]decan-1-one (Compound 1). To a solution of tert-butyl 2-(5-((4-([1,1′-biphenyl]-3-yl)-5-chloropyrimidin-2-yl)amino)pyridin-3-yl)-1-oxo-2,8-diazaspiro[4.5]decane-8-carboxylate (1-4) (90.0 mg, 147 μmol, 1.00 eq) in DCM (3 mL) was added TFA (1.54 g, 13.5 mmol, 1.00 mL, 91.7 eq) at 20° C. The mixture was stirred at 20° C. for 1 h. LCMS showed tert-butyl 2-(5-((4-([1,1′-biphenyl]-3-yl)-5-chloropyrimidin-2-yl)amino)pyridin-3-yl)-1-oxo-2,8-diazaspiro[4.5]decane-8-carboxylate (1-4) was consumed completely and the desired mass was detected. The reaction mixture was concentrated under reduced pressure to remove solvent. The residue was purified by prep-HPLC (column: Phenomenex Luna C18 75*30 mm*3 um; mobile phase: [water (FA)-ACN]; B %: 15%-45%, 8 min) to give the title compound (Compound 1) (25.2 mg, 49.3 μmol, 33.5% yield, 100% purity) as a white solid. LCMS (ES⁺): m/z 511.1 [M+H]⁺.

### Example 2. Synthesis of 1-(5-((5-chloro-4-(3′,4′-dichloro-[1,1′-biphenyl]-3-yl)pyrimidin-2-yl)amino)pyridin-3-yl)pyrrolidin-2-one (Compound 2)

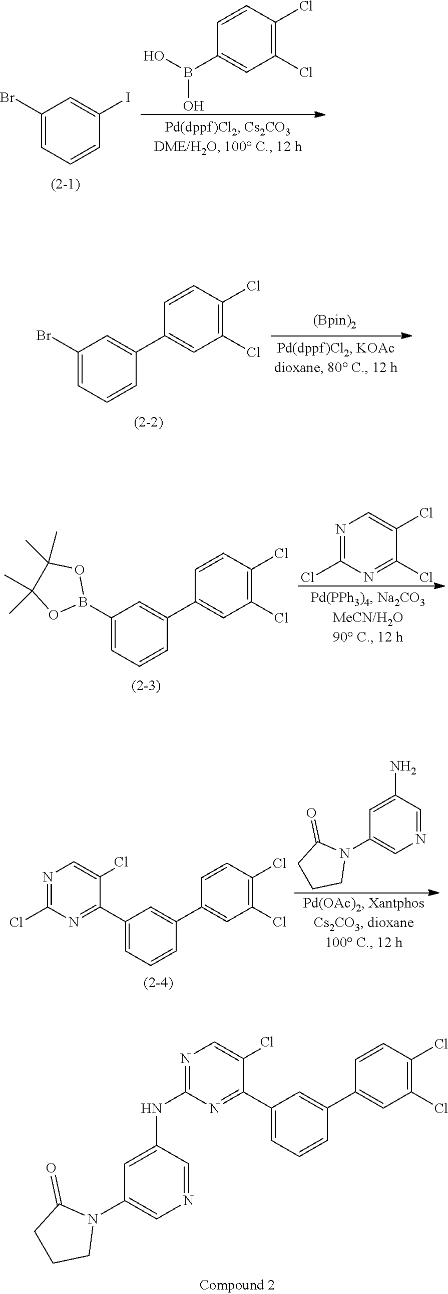

Synthesis of 3′-bromo-3,4-dichloro-1,1′-biphenyl (2-2). To a solution of 1-bromo-3-iodobenzene (2-1) (4.00 g, 14.1 mmol, 1.80 mL, 1.00 eq) in DME (30 mL) and H₂O (2.5 mL) were added (3,4-dichlorophenyl)boronic acid (2.70 g, 14.1 mmol, 1.00 eq), Cs₂CO₃ (13.8 g, 42.4 mmol, 3.00 eq) and Pd(dppf)Cl₂ (517 mg, 707 μmol, 0.0500 eq) at 20° C. under N₂. The mixture was stirred at 80° C. for 12 h under N₂ atmosphere. TLC (SiO₂, Petroleum ether:Ethyl acetate=1:0) showed 1-bromo-3-iodobenzene (2-1) was consumed completely and a new spot with a larger polarity formed. Then water (60 mL) was added to reaction, the aqueous phase was extracted with EtOAc (60 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 10:1) to give the title compound (2-2) (3.40 g, 11.3 mmol, 79.6% yield) as a white solid.

Synthesis of 2-(3′,4′-dichloro-[1,1′-biphenyl]-3-yl)-4,4,5,5-tetramethyl-1,3,2-dioxaborolane (2-3). To a solution of 3′-bromo-3,4-dichloro-1,1′-biphenyl (2-2) (3.00 g, 9.93 mmol, 1.00 eq) in dioxane (120 mL) were added (Bpin)₂ (3.03 g, 11.9 mmol, 1.20 eq), KOAc (1.95 g, 19.9 mmol, 2.00 eq) and Pd(dppf)Cl₂ (363 mg, 497 μmol, 0.0500 eq) at 20° C. under N₂. The mixture was stirred at 80° C. for 12 h under N₂ atmosphere. TLC (SiO₂, Petroleum ether:Ethyl acetate=20:1) indicated 3′-bromo-3,4-dichloro-1,1′-biphenyl (2-2) was consumed completely and a new spot with a little larger polarity was formed. Water (80 mL) was added to reaction, the aqueous phase was extracted with EtOAc (80 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 7:1) to give the title compound (2-3) (2.87 g, 8.22 mmol, 82.8% yield) as a green solid.

Synthesis of 2,5-dichloro-4-(3′,4′-dichloro-[1,1′-biphenyl]-3-yl)pyrimidine (2-4). To a solution of 2-(3′,4′-dichloro-[1,1′-biphenyl]-3-yl)-4,4,5,5-tetramethyl-1,3,2-dioxaborolane (2-3) (1.37 g, 3.92 mmol, 1.00 eq) in MeCN (8 mL) and H₂O (0.8 mL) were added 2,4,5-trichloropyrimidine (1.44 g, 7.85 mmol, 2.00 eq), Na₂CO₃ (832 mg, 7.85 mmol, 2.00 eq) and Pd(PPh₃)₄ (90.7 mg, 78.5 μmol, 0.0200 eq) at 20° C. under N₂. The mixture was stirred at 90° C. for 12 h under N₂ atmosphere. LCMS showed the desired mass was detected. Water (50 mL) was added to the reaction, the aqueous phase was extracted with EtOAc (50 mL*3), the combined organic layers were dried over Na₂SO₄, filtered and the filtrate was concentrated under reduced pressure to give a residue. The residue was purified by column chromatography (SiO₂, Petroleum ether:Ethyl acetate=1:0 to 8:1) to give the title compound (2-4) (1.40 g, 3.78 mmol, 96.3% yield) as a brown solid. LCMS (ES⁺): m/z 369.0 [M+H]⁺.

Synthesis of 1-(5-((5-chloro-4-(3′,4′-dichloro-[1,1′-biphenyl]-3-yl)pyrimidin-2-yl)amino)pyridin-3-yl)pyrrolidin-2-one (Compound 2). To a solution of 2,5-dichloro-4-(3′,4′-dichloro-[1,1′-biphenyl]-3-yl)pyrimidine (2-4) (340 mg, 919 μmol, 0.800 eq) in dioxane (15 mL) were added 1-(5-amino-3-pyridyl)pyrrolidin-2-one (448 mg, 1.26 mmol, 50.0% purity, 1.10 eq), Xantphos (19.9 mg, 34.5 μmol, 0.0300 eq), Cs₂CO₃ (748 mg, 2.30 mmol, 2.00 eq) and Pd(OAc)₂ (12.9 mg, 57.4 μmol, 0.0500 eq) at 20° C. under N₂. The mixture was stirred at 100° C. for 12 h under N₂ atmosphere. LCMS showed 2,5-dichloro-4-(3′,4′-dichloro-[1,1′-biphenyl]-3-yl)pyrimidine was consumed completely and the desired mass was detected. Water (8 mL) was added to reaction, the aqueous phase was extracted with EtOAc (8 mL*3), the combined organic layers were dried over Na₂SO₄, filtered and the filtrate was concentrated under reduced pressure. The residue was purified by prep-HPLC (column: Phenomenex Luna C18 75*30 mm*3 um; mobile phase: [water (FA)-ACN]; B %: 25%-65%, 8 min) to give the title compound (Compound 2) (35.8 mg, 69.2 μmol, 6.03% yield, 98.7% purity) as a white solid. LCMS (ES⁺): m/z 510.1 [M+H]⁺.

### Example 3. Synthesis of 1-(5-((5-chloro-4-(4′-methyl-[1,1′-biphenyl]-3-yl)pyrimidin-2-yl)amino)pyridin-3-yl)pyrrolidin-2-one (Compound 3)

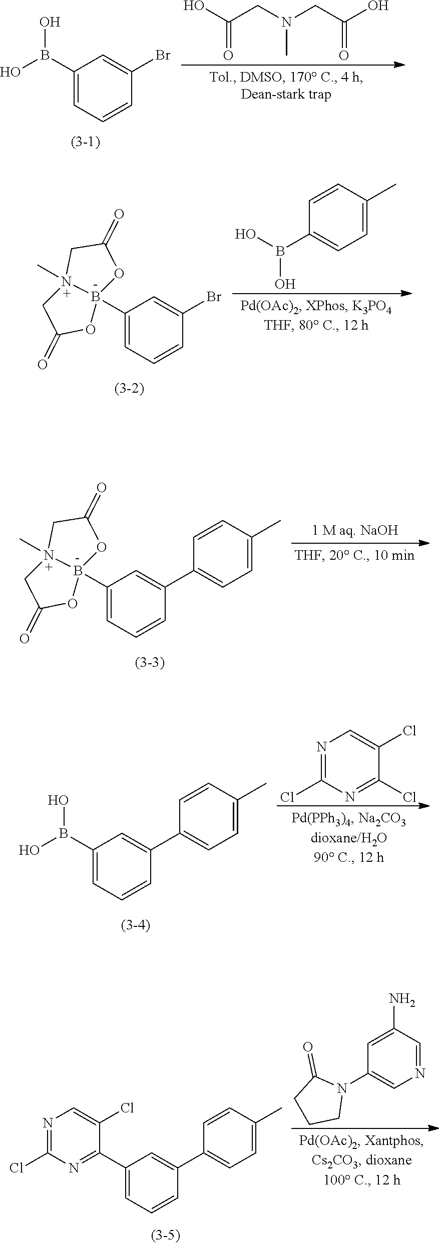

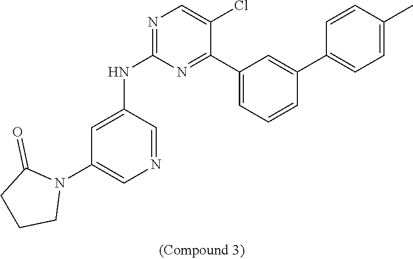 [SMILES: Cc1ccc(-c2cccc(-c3nc(Nc4cncc(N5CCCC5=O)c4)ncc3Cl)c2)cc1]

Synthesis of 8-(3-bromophenyl)-4-methyldihydro-414,814-[1,3,2]oxazaborolo[2,3-b][1,3,2]oxazaborole-2,6(3H,5H)-dione (3-2). To a solution of (3-bromophenyl)boronic acid (3-1) (3.00 g, 14.9 mmol, 1.00 eq) in toluene (130 mL) and DMSO (13 mL) was added 2-[carboxymethyl(methyl)amino]acetic acid (2.31 g, 15.7 mmol, 1.05 eq) at 20° C. The flask was fitted with a Dean-Stark trap and the Dean-Stark trap was fitted with a reflux condenser vented to ambient atmosphere. The stirred mixture was heated to 170° C. with azeotropic removal of water for 4 h. TLC (SiO₂, Petroleum ether:Ethyl acetate=1:1) indicated (3-bromophenyl)boronic acid (3-1) was consumed completely and one new spot with larger polarity than (3-bromophenyl)boronic acid (3-1) formed. The reaction mixture was concentrated under reduced pressure, adding water (30 mL) to the resulting residue, then filtered, the filter cake was dried under reduced pressure to give the title compound (3-2) (3.55 g, crude) as a brown solid.

Synthesis of 4-methyl-8-(4′-methyl-[1,1′-biphenyl]-3-yl)dihydro-414,814-[1,3,2]oxazaborolo[2,3-b][1,3,2]oxazaborole-2,6(3H,5H)-dione (3-3). To a solution of 8-(3-bromophenyl)-4-methyldihydro-414,814-[1,3,2]oxazaborolo[2,3-b][1,3,2]oxazaborole-2,6(3H,5H)-dione (3-2) (1.00 g, 3.21 mmol, 1.00 eq) in THF (100 mL) were added p-tolylboronic acid (654 mg, 4.81 mmol, 1.50 eq), K₃PO₄ (2.04 g, 9.62 mmol, 3.00 eq), XPhos (61.1 mg, 128 μmol, 0.0400 eq) and Pd(OAc)₂ (14.4 mg, 64.1 μmol, 0.0200 eq) at 20° C. under N₂. The mixture was stirred at 80° C. for 12 h under N₂ atmosphere. LCMS showed the desired mass was detected. Water (30 mL) was added to the reaction, the aqueous phase was extracted with EtOAc (30 mL*3), the combined organic layers were dried over Na₂SO₄, filtered and the filtrate was concentrated under reduced pressure to give a residue. Then to the residue was added MeOH (20 mL), filtered and the filter cake was dried under reduced pressure to give the title compound (3-3) (730 mg, 2.78 mmol, 86.6% yield) as a white solid. LCMS (ES⁺): m/z 324.0 [M+H]⁺.

Synthesis of (4′-methyl-[1,1′-biphenyl]-3-yl)boronic acid (3-4). To a solution of 4-methyl-8-(4′-methyl-[1,1′-biphenyl]-3-yl)dihydro-414,814-[1,3,2]oxazaborolo[2,3-b][1,3,2]oxazaborole-2,6(3H,5H)-dione (3-3) (730 mg, 2.78 mmol, 1.00 eq) in THF (10 mL) was added NaOH (1.00 M, 8.37 mL, 3.00 eq) at 20° C. The mixture was stirred at 20° C. for 10 min. TLC (SiO₂, Petroleum ether:Ethyl acetate=3:1) indicated 4-methyl-8-(4′-methyl-[1,1′-biphenyl]-3-yl)dihydro-414,814-[1,3,2]oxazaborolo[2,3-b][1,3,2]oxazaborole-2,6(3H,5H)-dione (3-3) was consumed completely. TLC (SiO₂, Petroleum ether:Ethyl acetate=10:1) indicated a new spot with lower polarity than 4-methyl-8-(4′-methyl-[1,1′-biphenyl]-3-yl)dihydro-414,814-[1,3,2]oxazaborolo[2,3-b][1,3,2]oxazaborole-2,6(3H,5H)-dione (3-3) formed. The reaction mixture was concentrated under reduced pressure. The residue was purified by prep-TLC (SiO₂, Petroleum ether:Ethyl acetate=3:1) to give the title compound (3-4) (245 mg, 1.16 mmol, 41.4% yield) as a white solid.

Synthesis of 2,5-dichloro-4-(4′-methyl-[1,1′-biphenyl]-3-yl)pyrimidine (3-5). To a solution of (4′-methyl-[1,1′-biphenyl]-3-yl)boronic acid (3-4) (245 mg, 1.16 mmol, 1.00 eq) in dioxane (8 mL) and H₂O (0.8 mL) were added 2,4,5-trichloropyrimidine (424 mg, 2.31 mmol, 2.00 eq), Pd(PPh₃)₄ (26.7 mg, 23.1 μmol, 0.0200 eq) and Na₂CO₃ (245 mg, 2.31 mmol, 2.00 eq) at 20° C. under N₂. The mixture was stirred at 90° C. for 12 h under N₂ atmosphere. LCMS showed the desired mass was detected. Water (5 mL) was added to reaction, the aqueous phase was extracted with EtOAc (5 mL*3), the combined organic layers were dried over Na₂SO₄, filtered and the filtrate was concentrated under reduced pressure. The residue was purified by prep-TLC (SiO₂, Petroleum ether:Ethyl acetate=10:1) to give the title compound (3-5) (200 mg, 635 μmol, 54.9% yield) as a white solid. LCMS (ES⁺): m/z 315.1 [M+H]⁺.

Synthesis of 1-(5-((5-chloro-4-(4′-methyl-[1,1′-biphenyl]-3-yl)pyrimidin-2-yl)amino)pyridin-3-yl)pyrrolidin-2-one (Compound 3). To a solution of 2,5-dichloro-4-(4′-methyl-[1,1′-biphenyl]-3-yl)pyrimidine (3-5) (200 mg, 635 μmol, 0.80 eq) in dioxane (8 mL) were added 1-(5-amino-3-pyridyl)pyrrolidin-2-one (309 mg, 872 μmol, 50.0% purity, 1.10 eq), Cs₂CO₃ (517 mg, 1.59 mmol, 2.00 eq), Pd(OAc)₂ (8.90 mg, 39.7 μmol, 0.0500 eq) and Xantphos (13.8 mg, 23.8 μmol, 0.0300 eq) at 20° C. under N₂. The mixture was stirred at 100° C. for 12 h under N₂ atmosphere. LCMS showed 2,5-dichloro-4-(4′-methyl-[1,1′-biphenyl]-3-yl)pyrimidine (3-5) was consumed completely and the desired mass was detected. The reaction mixture was concentrated under reduced pressure. The residue was purified by prep-HPLC (column: Phenomenex Luna C18 75*30 mm*3 um; mobile phase: [water (FA)-ACN]; B %: 40%-80%, 8 min) to give the title compound (Compound 3) (53.0 mg, 115 μmol, 14.5% yield, 99.0% purity) as a white solid. LCMS (ES⁺): m/z 456.1 [M+H]⁺.

### Example 4. Synthesis of 1-(5-((5-chloro-4-(4′-chloro-[1,1′-biphenyl]-3-yl)pyrimidin-2-yl)amino)pyridin-3-yl)pyrrolidin-2-one (Compound 4)

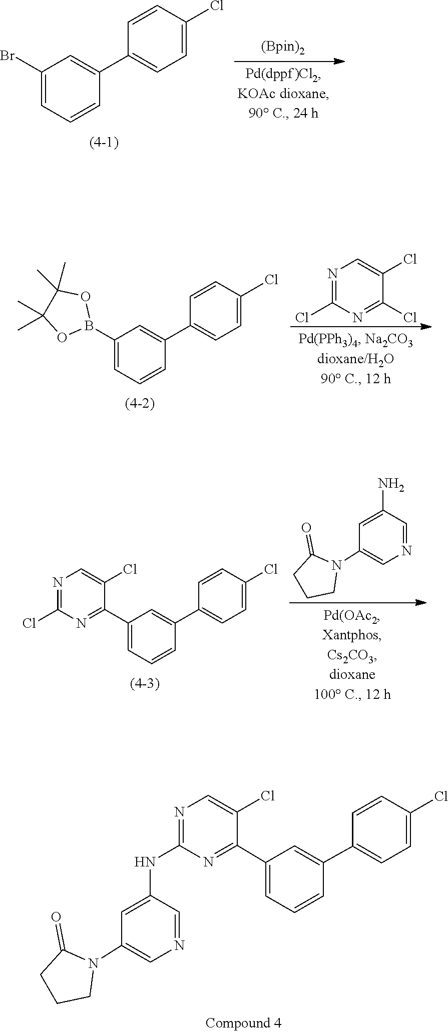

Synthesis of 2-(4′-chloro-[1,1′-biphenyl]-3-yl)-4,4,5,5-tetramethyl-1,3,2-dioxaborolane (4-2). To a solution of 3-bromo-4′-chloro-1,1′-biphenyl (4-1) (3.00 g, 11.2 mmol, 1.00 eq) in dioxane (150 mL) were added 4,4,5,5-tetramethyl-2-(4,4,5,5-tetramethyl-1,3,2-dioxaborolan-2-yl)-1,3,2-dioxaborolane (3.42 g, 13.5 mmol, 1.20 eq), KOAc (2.20 g, 22.4 mmol, 2.00 eq) and Pd(dppf)Cl₂ (410 mg, 561 μmol, 0.0500 eq) at 20° C. under N₂. The mixture was stirred at 90° C. for 24 h under N₂ atmosphere. TLC (SiO₂, Petroleum ether:Ethyl acetate=20:1) indicated 3-bromo-4′-chloro-1,1′-biphenyl (4-1) was consumed completely and a new spot with a little larger polarity than 3-bromo-4′-chloro-1,1′-biphenyl (4-1) formed. Water (100 mL) was added to reaction, the aqueous phase was extracted with EtOAc (100 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 8:1) to give the title compound (4-2) (2.84 g, 9.03 mmol, 80.5% yield) as a brown solid.

Synthesis of 2,5-dichloro-4-(4′-chloro-[1,1′-biphenyl]-3-yl)pyrimidine (4-3). To a solution of 2-(4′-chloro-[1,1′-biphenyl]-3-yl)-4,4,5,5-tetramethyl-1,3,2-dioxaborolane (4-2) (1.00 g, 3.18 mmol, 1.00 eq) in dioxane (50 mL) and H₂O (5 mL) were added 2,4,5-trichloropyrimidine (1.17 g, 6.36 mmol, 2.00 eq), Pd(PPh₃)₄ (73.5 mg, 63.6 μmol, 0.0200 eq) and Na₂CO₃ (674 mg, 6.36 mmol, 2.00 eq) at 20° C. The mixture was stirred at 90° C. for 12 h under N₂ atmosphere. LCMS showed 2-(4′-chloro-[1,1′-biphenyl]-3-yl)-4,4,5,5-tetramethyl-1,3,2-dioxaborolane (4-2) was consumed completely and the desired mass was detected. Water (30 mL) was added to reaction, the aqueous phase was extracted with EtOAc (30 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 10:1) to give the title compound (4-3) (780 mg, 2.32 mmol, 73.1% yield) as a white solid. LCMS (ES⁺): m/z 334.9 [M+H]⁺.

Synthesis of 1-(5-((5-chloro-4-(4′-chloro-[1,1′-biphenyl]-3-yl)pyrimidin-2-yl)amino)pyridin-3-yl)pyrrolidin-2-one (Compound 4). To a solution of 2,5-dichloro-4-(4′-chloro-[1,1′-biphenyl]-3-yl)pyrimidine (4-3) (400 mg, 1.19 mmol, 0.800 eq) in dioxane (15 mL) were added 1-(5-amino-3-pyridyl)pyrrolidin-2-one (581 mg, 1.64 mmol, 50.0% purity, 1.10 eq), Xantphos (25.9 mg, 44.7 μmol, 0.0300 eq), Cs₂CO₃ (971 mg, 2.98 mmol, 2.00 eq) and Pd(OAc)₂ (16.7 mg, 74.5 μmol, 0.0500 eq) at 20° C. under N₂. The mixture was stirred at 100° C. for 12 h under N₂ atmosphere. LCMS showed 2,5-dichloro-4-(4′-chloro-[1,1′-biphenyl]-3-yl)pyrimidine (4-3) was consumed completely and the desired mass was detected. Water (10 mL) was added to reaction, the aqueous phase was extracted with EtOAc (8 mL*3), the combined organic layers were dried over Na₂SO₄, filtered and the filtrate was concentrated under reduced pressure. The residue was purified by prep-HPLC (column: Phenomenex C18 75*30 mm*3 um; mobile phase: [water (HCl)-ACN]; B %: 60%-90%, 8 min) to give the title compound (Compound 4) (147 mg, 307 μmol, 20.6% yield, 99.6% purity) as a white solid. LCMS (ES⁺): m/z 476.0 [M+H]⁺.

### Example 5. Synthesis of 1-(5-((5-chloro-4-(4′-methoxy-[1,1′-biphenyl]-3-yl)pyrimidin-2-yl)amino)pyridin-3-yl)pyrrolidin-2-one (Compound 5)

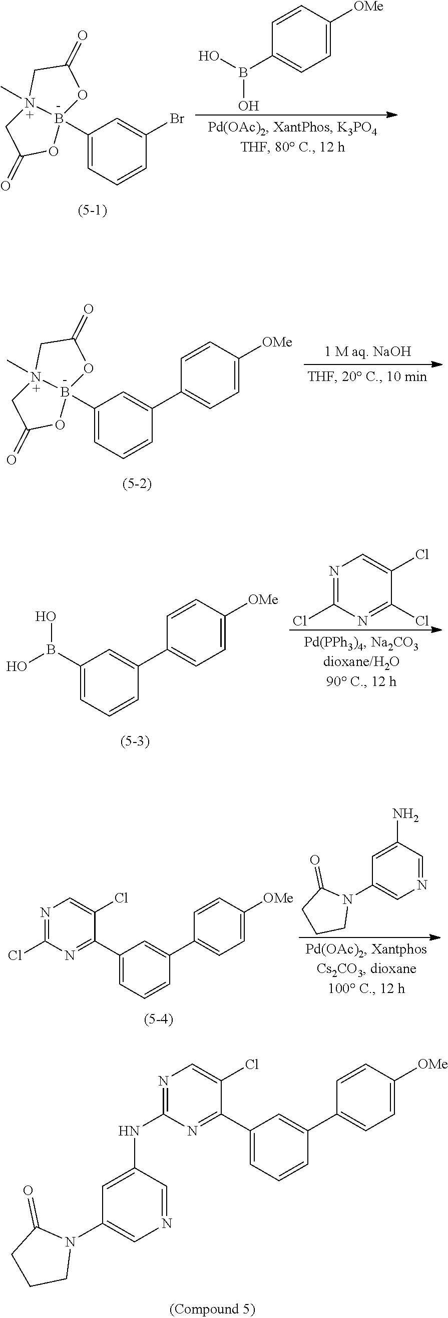

Synthesis of 8-(4′-methoxy-[1,1′-biphenyl]-3-yl)-4-methyldihydro-414,814-[1,3,2]oxazaborolo[2,3-b][1,3,2]oxazaborole-2,6(3H,5H)-dione (5-2). To a solution of 8-(3-bromophenyl)-4-methyldihydro-414,814-[1,3,2]oxazaborolo[2,3-b][1,3,2]oxazaborole-2,6(3H,5H)-dione (5-1) (which is prepared as described in Example 3) (4.20 g, 13.5 mmol, 1.00 eq) in THF (100 mL) were added (4-methoxyphenyl)boronic acid (3.07 g, 20.2 mmol, 1.50 eq), K₃PO₄ (8.57 g, 40.4 mmol, 3.00 eq), Pd(OAc)₂ (60.5 mg, 269 μmol, 0.0200 eq) and Xantphos (312 mg, 539 μmol, 0.0400 eq) at 20° C. under N₂. The mixture was stirred at 80° C. for 12 h under N₂ atmosphere. LCMS showed 8-(3-bromophenyl)-4-methyldihydro-414,814-[1,3,2]oxazaborolo[2,3-b][1,3,2]oxazaborole-2,6(3H,5H)-dione (5-1) was consumed completely and the desired mass was detected. Water (100 mL) was added to the reaction, the aqueous phase was extracted with EtOAc (100 mL*3), the combined organic layers were dried over Na₂SO₄, filtered and the filtrate was concentrated under reduced pressure to give a residue. Then to the residue was added MeOH (20 mL), filtered and the filter cake was dried under reduced pressure to give the title compound (5-2) as a white solid (730 mg). The filtrate was concentrated under reduced pressure to give a residue. (300 mg). The residue (300 mg) was purified by prep-TLC (SiO₂, Petroleum ether:Ethyl acetate=0:1) to give the title compound (5-2) (70.0 mg). Total the title compound (5-2) (800 mg, 2.35 mmol, 17.5% yield) was obtained as a white solid. LCMS (ES⁺): m/z 340.1 [M+H]⁺.

Synthesis of (4′-methoxy-[1,1′-biphenyl]-3-yl)boronic acid (5-3). To a solution of 8-(4′-methoxy-[1,1′-biphenyl]-3-yl)-4-methyldihydro-414,814-[1,3,2]oxazaborolo[2,3-b][1,3,2]oxazaborole-2,6(3H,5H)-dione (5-2) (800 mg, 2.35 mmol, 1.00 eq) in THF (10 mL) were added NaOH (1.00 M, 7.06 mL, 3.00 eq) at 20° C. The mixture was stirred at 20° C. for 10 min. TLC (SiO₂, Petroleum ether:Ethyl acetate=2:1) indicated 8-(4′-methoxy-[1,1′-biphenyl]-3-yl)-4-methyldihydro-414,814-[1,3,2]oxazaborolo[2,3-b][1,3,2]oxazaborole-2,6(3H,5H)-dione (5-2) was consumed completely and a new spot with lower polarity formed. The reaction mixture was concentrated under reduced pressure. The residue was purified by prep-TLC (SiO₂, Petroleum ether:Ethyl acetate=2:1) to give the title compound (5-3) (352 mg, 1.54 mmol, 65.6% yield) as a white solid.

Synthesis of 2,5-dichloro-4-(4′-methoxy-[1,1′-biphenyl]-3-yl)pyrimidine (5-4). To a solution of (4′-methoxy-[1,1′-biphenyl]-3-yl)boronic acid (5-3) (352 mg, 1.54 mmol, 1.00 eq) in dioxane (8 mL) and H₂O (0.8 mL) were added 2,4,5-trichloropyrimidine (566 mg, 3.09 mmol, 2.00 eq), Na₂CO₃ (327 mg, 3.09 mmol, 2.00 eq) and Pd(PPh₃)₄ (35.7 mg, 30.9 μmol, 0.0200 eq) at 20° C. under N₂. The mixture was stirred at 90° C. for 12 h under N₂ atmosphere. LCMS showed the desired mass was detected. The residue was purified by prep-TLC (SiO₂, Petroleum ether:Ethyl acetate=5:1) to give the title compound (5-4) (220 mg, 664 μmol, 43.0% yield) as a white solid. LCMS (ES⁺): m/z 331.2 [M+H]⁺.

Synthesis of 1-(5-((5-chloro-4-(4′-methoxy-[1,1′-biphenyl]-3-yl)pyrimidin-2-yl)amino)pyridin-3-yl)pyrrolidin-2-one (Compound 5). To a solution of 2,5-dichloro-4-(4′-methoxy-[1,1′-biphenyl]-3-yl)pyrimidine (5-4) (220 mg, 664 μmol, 0.800 eq) in dioxane (8 mL) were added 1-(5-amino-3-pyridyl)pyrrolidin-2-one (324 mg, 913 μmol, 50.0% purity, 1.10 eq), Cs₂CO₃ (541 mg, 1.66 mmol, 2.00 eq), Pd(OAc)₂ (9.32 mg, 41.5 μmol, 0.0500 eq) and Xantphos (14.4 mg, 24.9 μmol, 0.0300 eq) at 20° C. under N₂. The mixture was stirred at 100° C. for 12 h under N₂ atmosphere. LCMS showed 2,5-dichloro-4-(4′-methoxy-[1,1′-biphenyl]-3-yl)pyrimidine (5-4) was consumed completely and the desired mass was detected. The residue was purified by prep-HPLC (column: Phenomenex luna C18 100*40 mm*3 um; mobile phase: [water (FA)-ACN]; B %: 40%-60%, 8 min) to give the title compound (Compound 5) (39.2 mg, 82.8 μmol, 9.97% yield, 99.7% purity) as a white solid. LCMS (ES⁺): m/z 472.1 [M+H]⁺.

### Example 6. Synthesis of 1-(5-((5-chloro-4-(6-chloro-[1,1′-biphenyl]-3-yl)pyrimidin-2-yl)amino)pyridin-3-yl)pyrrolidin-2-one (Compound 6)

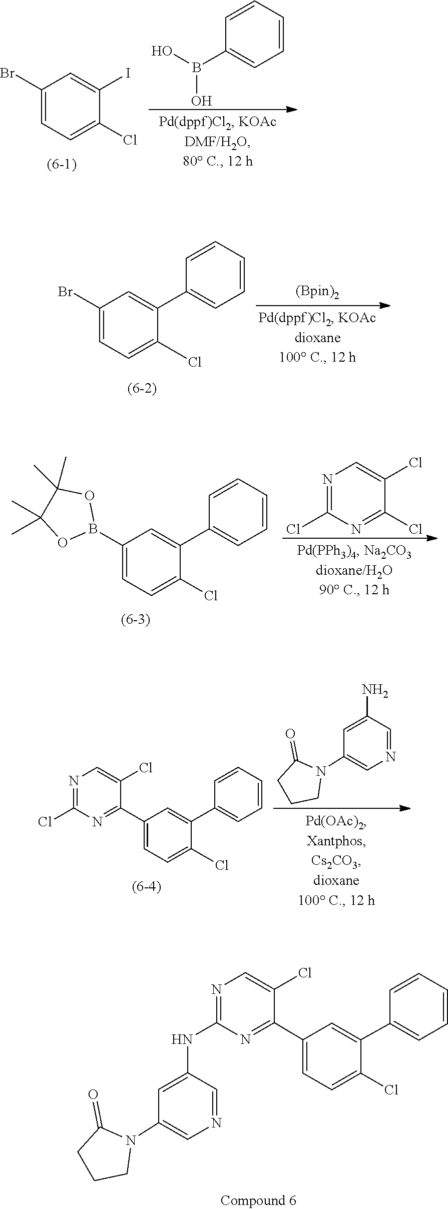

Synthesis of 5-bromo-2-chloro-1,1′-biphenyl (6-2). To a solution of 4-bromo-1-chloro-2-iodobenzene (6-1) (1.00 g, 3.15 mmol, 1.00 eq) in DMF (25 mL) and H₂O (2.5 mL) were added phenylboronic acid (576 mg, 4.73 mmol, 1.50 eq), KOAc (619 mg, 6.30 mmol, 2.00 eq) and Pd(dppf)Cl₂ (231 mg, 315 μmol, 0.100 eq) at 20° C. under N₂. The mixture was stirred at 80° C. for 12 h under N₂ atmosphere. TLC (SiO₂, Petroleum ether:Ethyl acetate=1:0) indicated 4-bromo-1-chloro-2-iodobenzene (6-1) was consumed completely and a new spot with a little larger polarity than 4-bromo-1-chloro-2-iodobenzene (6-1) formed. Water (25 mL) was added to the reaction, the aqueous phase was extracted with EtOAc (20 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 1:0) to give the title compound (6-2) (560 mg, 2.09 mmol, 66.4% yield) as a colorless oil.

Synthesis of 2-(6-chloro-[1,1′-biphenyl]-3-yl)-4,4,5,5-tetramethyl-1,3,2-dioxaborolane (6-3). To a solution of 5-bromo-2-chloro-1,1′-biphenyl (6-2) (560 mg, 2.09 mmol, 1.00 eq) in dioxane (15 mL) were added (Bpin)₂ (638 mg, 2.51 mmol, 1.20 eq), Pd(dppf)Cl₂ (76.6 mg, 105 μmol, 0.0500 eq) and KOAc (411 mg, 4.19 mmol, 2.00 eq) at 20° C. under N₂. The mixture was stirred at 80° C. for 12 h under N₂ atmosphere. TLC (SiO₂, Petroleum ether:Ethyl acetate=20:1) indicated 5-bromo-2-chloro-1,1′-biphenyl (6-2) remained and a new spot with larger polarity than 5-bromo-2-chloro-1,1′-biphenyl (6-2) formed. Water (10 mL) was added to the reaction, the aqueous phase was extracted with EtOAc (10 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 8:1) to give the title compound (6-3) (470 mg, 1.49 mmol, 71.4% yield) as a white solid.

Synthesis of 2,5-dichloro-4-(6-chloro-[1,1′-biphenyl]-3-yl)pyrimidine (6-4). To a solution of 2-(6-chloro-[1,1′-biphenyl]-3-yl)-4,4,5,5-tetramethyl-1,3,2-dioxaborolane (6-3) (470 mg, 1.49 mmol, 1.00 eq) in dioxane (15 mL) and H₂O (1.5 mL) were added 2,4,5-trichloropyrimidine (548 mg, 2.99 mmol, 2.00 eq), Na₂CO₃ (317 mg, 2.99 mmol, 2.00 eq) and Pd(PPh₃)₄ (34.5 mg, 29.9 μmol, 0.0200 eq) at 20° C. under N₂. The mixture was stirred at 90° C. for 12 h under N₂ atmosphere. LCMS showed 2-(6-chloro-[1,1′-biphenyl]-3-yl)-4,4,5,5-tetramethyl-1,3,2-dioxaborolane (6-3) was consumed completely and the desired mass was detected. The residue was purified by column chromatography (SiO₂, Petroleum ether:Ethyl acetate=1:0 to 8:1) to give the title compound (6-4) (260 mg, 775 μmol, 51.9% yield) as a white solid. LCMS (ES⁺): m/z 334.9 [M+H]⁺.

Synthesis of 1-(5-((5-chloro-4-(6-chloro-[1,1′-biphenyl]-3-yl)pyrimidin-2-yl)amino)pyridin-3-yl)pyrrolidin-2-one (Compound 6). To a solution of 2,5-dichloro-4-(6-chloro-[1,1′-biphenyl]-3-yl)pyrimidine (6-4) (260 mg, 775 μmol, 0.800 eq) in dioxane (8 mL) were added 1-(5-amino-3-pyridyl)pyrrolidin-2-one (378 mg, 1.07 mmol, 50.0% purity, 1.10 eq), Xantphos (16.8 mg, 29.1 μmol, 0.0300 eq), Cs₂CO₃ (631 mg, 1.94 mmol, 2.00 eq) and Pd(OAc)₂ (10.9 mg, 48.4 μmol, 0.0500 eq) at 20° C. under N₂. The mixture was stirred at 100° C. for 12 h under N₂ atmosphere. LCMS showed 2,5-dichloro-4-(6-chloro-[1,1′-biphenyl]-3-yl)pyrimidine (6-4) was consumed completely and the desired mass was detected. Water (5 mL) was added to reaction, the aqueous phase was extracted with EtOAc (5 mL*3), the combined organic layers were dried over Na₂SO₄, filtered and the filtrate was concentrated under reduced pressure. The residue was purified by prep-HPLC (column: Phenomenex Luna C18 75*30 mm*3 um; mobile phase: [water (FA)-ACN]; B %: 30%-80%, 8 min) to give the title compound (Compound 6) (85.8 mg, 177 μmol, 18.3% yield, 98.8% purity) as a yellow solid. LCMS (ES⁺): m/z 476.0 [M+H]⁺.

### Example 7. Synthesis of 1-(5-((5-chloro-4-(6-methyl-[1,1′-biphenyl]-3-yl)pyrimidin-2-yl)amino)pyridin-3-yl)pyrrolidin-2-one (Compound 7)

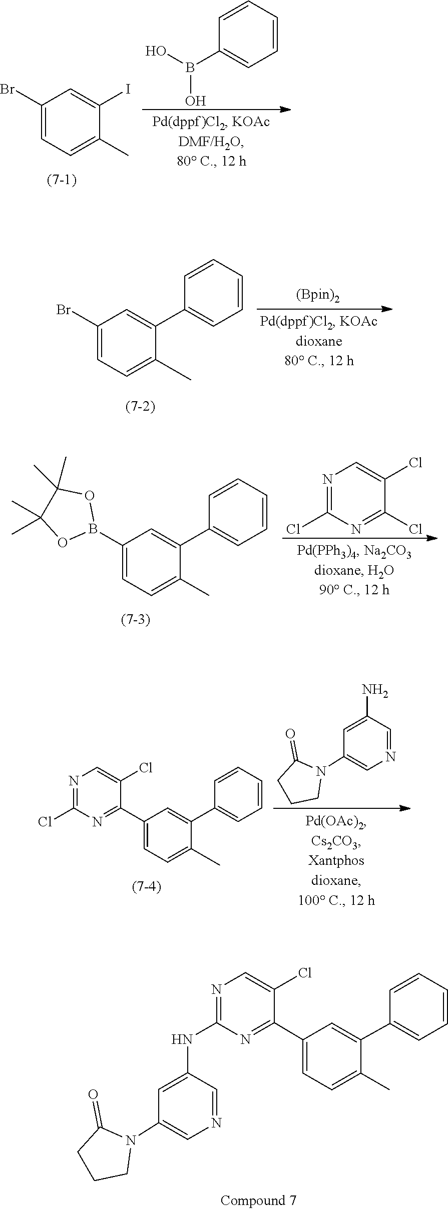

Synthesis of 5-bromo-2-methyl-1,1′-biphenyl (7-2). To a solution of 4-bromo-2-iodo-1-methylbenzene (7-1) (3.00 g, 10.1 mmol, 1.00 eq) in DMF (30 mL) and H₂O (3 mL) were added phenylboronic acid (1.85 g, 15.2 mmol, 1.50 eq), AcOK (1.98 g, 20.2 mmol, 2.00 eq) and Pd(dppf)Cl₂ (739 mg, 1.01 mmol, 0.100 eq) at 20° C. under N₂ atmosphere. The mixture was stirred at 80° C. for 12 h. No desired mass was detected by LCMS. TLC (SiO₂, Petroleum ether:Ethyl acetate=10:1) indicated 4-bromo-2-iodo-1-methylbenzene (7-1) was consumed completely and one new spot with a little larger polarity formed. Water (30 mL) was added to the mixture at 20° C. Then the reaction mixture was extracted with EtOAc (60 mL). The organic layer was dried over anhydrous sodium sulfate, filtered and the filtrate was concentrated under reduced pressure to give a residue. The residue was purified by column chromatography (SiO₂, Petroleum ether:Ethyl acetate=0:1 to 20:1) to give the title compound (7-2) (1.80 g, 7.28 mmol, 72.1% yield) as a yellow oil.

Synthesis of 4,4,5,5-tetramethyl-2-(6-methyl-[1,1′-biphenyl]-3-yl)-1,3,2-dioxaborolane (7-3). To a solution of 5-bromo-2-methyl-1,1′-biphenyl (7-2) (1.80 g, 7.28 mmol, 1.00 eq) in dioxane (40 mL) were added 4,4,5,5-tetramethyl-2-(4,4,5,5-tetramethyl-1,3,2-dioxaborolan-2-yl)-1,3,2-dioxaborolane (2.22 g, 8.74 mmol, 1.20 eq), KOAc (1.43 g, 14.6 mmol, 2.00 eq) and Pd(dppf)Cl₂ (266 mg, 364 μmol, 0.0500 eq) at 20° C. under atmosphere. The mixture was stirred at 80° C. for 12 h. LCMS showed 5-bromo-2-methyl-1,1′-biphenyl (7-2) was consumed completely and the desired mass was detected. The reaction mixture was concentrated under reduced pressure to remove solvent. The residue was purified by column chromatography (SiO₂, Petroleum ether:Ethyl acetate=1:0 to 10:1) to give the title compound (7-3) (2.00 g, 6.80 mmol, 93.3% yield) as a yellow oil. LCMS (ES⁺): m/z 295.1 [M+H]⁺.

Synthesis of 2,5-dichloro-4-(6-methyl-[1,1′-biphenyl]-3-yl)pyrimidine (7-4). To a solution of 4,4,5,5-tetramethyl-2-(6-methyl-[1,1′-biphenyl]-3-yl)-1,3,2-dioxaborolane (7-3) (2.00 g, 6.80 mmol, 1.00 eq) in dioxane (30 mL) and H₂O (3 mL) were added 2,4,5-trichloropyrimidine (2.49 g, 13.6 mmol, 2.00 eq), Pd(PPh₃)₄ (157 mg, 136 μmol, 0.0200 eq) and Na₂CO₃ (1.44 g, 13.6 mmol, 2.00 eq) at 20° C. under N₂ atmosphere. The mixture was stirred at 90° C. for 12 h. LCMS showed 4,4,5,5-tetramethyl-2-(6-methyl-[1,1′-biphenyl]-3-yl)-1,3,2-dioxaborolane (7-3) was consumed completely and the desired mass was detected. Water (30 mL) was added to the mixture at 20° C. Then the mixture was extracted with EtOAc (60 mL). The organic layer was dried over anhydrous sodium sulfate, filtered and the filtrate was concentrated under reduced pressure to give a residue. The residue was purified by column chromatography (SiO₂, Petroleum ether:Ethyl acetate=1:0 to 10:1) to give the title compound (7-4) (1.00 g, 3.17 mmol, 46.7% yield) as a white solid. LCMS (ES⁺): m/z 315.0 [M+H]⁺.

Synthesis of 1-(5-((5-chloro-4-(6-methyl-[1,1′-biphenyl]-3-yl)pyrimidin-2-yl)amino)pyridin-3-yl)pyrrolidin-2-one (Compound 7). To a solution of 2,5-dichloro-4-(6-methyl-[1,1′-biphenyl]-3-yl)pyrimidine (7-4) (400 mg, 1.27 mmol, 0.800 eq) in dioxane (5 mL) were added 1-(5-amino-3-pyridyl)pyrrolidin-2-one (618 mg, 1.74 mmol, 50.0% purity, 1.10 eq), Cs₂CO₃ (1.03 g, 3.17 mmol, 2.00 eq), Pd(OAc)₂ (17.8 mg, 79.3 μmol, 0.0500 eq) and Xantphos (27.5 mg, 47.6 μmol, 0.0300 eq) at 20° C. under N₂ atmosphere. The mixture was stirred at 100° C. for 12 h. LCMS showed 2,5-dichloro-4-(6-methyl-[1,1′-biphenyl]-3-yl)pyrimidine (7-4) was consumed completely and the desired mass was detected. The reaction mixture was concentrated under reduced pressure to remove solvent. The residue was purified by prep-HPLC (column: Phenomenex Luna C18 75*30 mm*3 um; mobile phase: [water (FA)-ACN]; B %: 40%-80%, 8 min) to give the title compound (Compound 7) (153 mg, 329 μmol, 20.7% yield, 98.1% purity) as a yellow solid. LCMS (ES⁺): m/z 456.0 [M+H]⁺.

### Example 8. Synthesis of 1-(5-((5-chloro-4-(6-methoxy-[1,1′-biphenyl]-3-yl)pyrimidin-2-yl)amino)pyridin-3-yl)pyrrolidin-2-one (Compound 8)

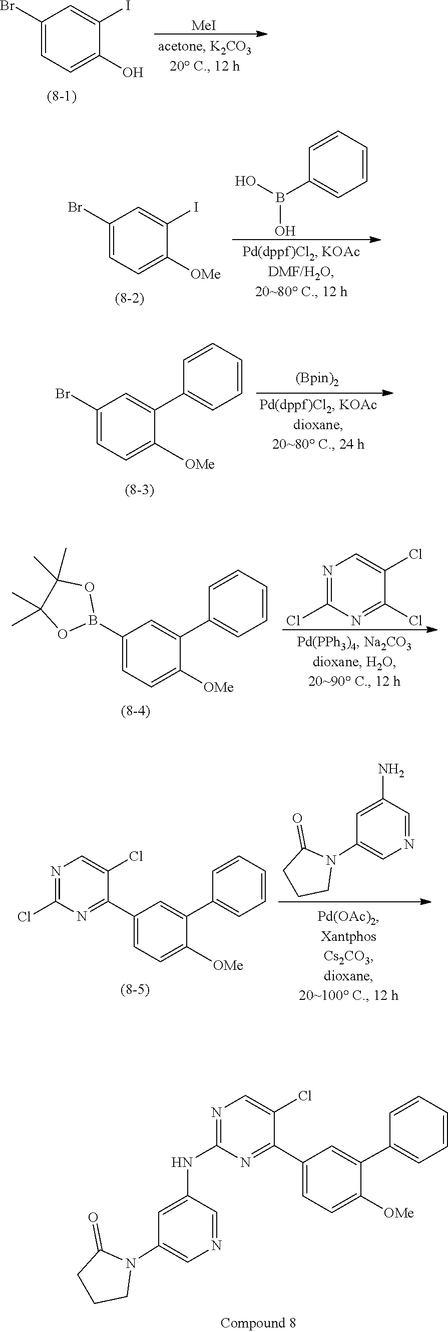

Synthesis of 4-bromo-2-iodo-1-methoxybenzene (8-2). To a solution of 4-bromo-2-iodophenol (8-1) (3.00 g, 10.0 mmol, 1.00 eq) in acetone (30 mL) were added K₂CO₃ (1.80 g, 13.0 mmol, 1.30 eq) and Mel (1.57 g, 11.0 mmol, 687 μL, 1.10 eq) at 20° C. The mixture was stirred at 20° C. for 12 h. TLC (SiO₂, Ethyl acetate:Petroleum ether=0:1) indicated ˜6% of 4-bromo-2-iodophenol (8-1) remained, and one major new spot with lower polarity was detected. The reaction mixture was concentrated under reduced pressure to remove solvent. The residue was purified by column chromatography (SiO₂, Ethyl acetate:Petroleum ether=0:1 to 1:99) to give the title compound (8-2) (2.34 g, 7.48 mmol, 74.5% yield) as a yellow oil.

Synthesis of 5-bromo-2-methoxy-1,1′-biphenyl (8-3). To a solution of 4-bromo-2-iodo-1-methoxybenzene (8-2) (2.30 g, 7.35 mmol, 1.00 eq) in DMF (30 mL) and H₂O (3 mL) were added KOAc (1.44 g, 14.7 mmol, 2.00 eq), phenylboronic acid (1.34 g, 11.0 mmol, 1.50 eq) and Pd(dppf)Cl₂ (538 mg, 735 μmol, 0.100 eq) at 20° C. under N₂ atmosphere. The mixture was stirred at 80° C. for 12 h. TLC (SiO₂, Ethyl acetate:Petroleum ether=1:20) indicated 4-bromo-2-iodo-1-methoxybenzene (8-2) was consumed completely and one new spot formed. Water (30 mL) was added to the mixture at 20° C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:0 to 20:1) to give the title compound (8-3) (1.45 g, 5.51 mmol, 75.0% yield) as a colorless oil.

Synthesis of 2-(6-methoxy-[1,1′-biphenyl]-3-yl)-4,4,5,5-tetramethyl-1,3,2-dioxaborolane (8-4). To a solution of 5-bromo-2-methoxy-1,1′-biphenyl (8-3) (1.40 g, 5.32 mmol, 1.00 eq) in dioxane (30 mL) were added KOAc (1.04 g, 10.6 mmol, 2.00 eq), 4,4,5,5-tetramethyl-2-(4,4,5,5-tetramethyl-1,3,2-dioxaborolan-2-yl)-1,3,2-dioxaborolane (1.62 g, 6.38 mmol, 1.20 eq) and Pd(dppf)Cl₂ (195 mg, 266 μmol, 0.0500 eq) at 20° C. under N₂ atmosphere. The mixture was stirred at 80° C. for 12 h. LCMS showed ˜32.3% of 5-bromo-2-methoxy-1,1′-biphenyl (8-3) was remaining and desired mass was detected. Then 4,4,5,5-tetramethyl-2-(4,4,5,5-tetramethyl-1,3,2-dioxaborolan-2-yl)-1,3,2-dioxaborolane (1.62 g), KOAc (502 mg) and Pd(dppf)Cl₂ (195 mg) were added to the mixture. The mixture was stirred at 80° C. for 12 h. LCMS showed ˜13.9% of 5-bromo-2-methoxy-1,1′-biphenyl (8-3) was remaining and desired mass was detected. The reaction mixture was concentrated under reduced pressure to remove solvent. The residue was purified by column chromatography (SiO₂, Petroleum ether:Ethyl acetate=1:0 to 20:1) to give the title compound (8-4) (1.70 g, 5.20 mmol, 97.8% yield, 94.9% purity) as a white solid. LCMS (ES⁺): m/z 311.4 [M+H]⁺.

Synthesis of 2,5-dichloro-4-(6-methoxy-[1,1′-biphenyl]-3-yl)pyrimidine (8-5). To a solution of 2-(6-methoxy-[1,1′-biphenyl]-3-yl)-4,4,5,5-tetramethyl-1,3,2-dioxaborolane (8-4) (700 mg, 2.26 mmol, 1.00 eq) in dioxane (15 mL) and H₂O (1.5 mL) were added Pd(PPh₃)₄ (52.2 mg, 45.1 μmol, 0.0200 eq), 2,4,5-trichloropyrimidine (828 mg, 4.51 mmol, 2.00 eq) and Na₂CO₃ (478 mg, 4.51 mmol, 2.00 eq) at 20° C. under N₂ atmosphere. The mixture was stirred at 90° C. for 12 h. LCMS showed 2-(6-methoxy-[1,1′-biphenyl]-3-yl)-4,4,5,5-tetramethyl-1,3,2-dioxaborolane (8-4) was consumed completely and the desired mass was detected. The reaction mixture was concentrated under reduced pressure to remove solvent. The residue was purified by prep-TLC (SiO₂, Petroleum ether:Ethyl acetate=10:1) to give the title compound (8-5) (530 mg, 1.60 mmol, 70.9% yield) as a white solid. LCMS (ES⁺): m/z 331.2 [M+H]⁺.

Synthesis of 1-(5-((5-chloro-4-(6-methoxy-[1,1′-biphenyl]-3-yl)pyrimidin-2-yl)amino)pyridin-3-yl)pyrrolidin-2-one (Compound 8). To a solution of 2,5-dichloro-4-(6-methoxy-[1,1′-biphenyl]-3-yl)pyrimidine (8-5) (200 mg, 604 μmol, 0.800 eq) in dioxane (4 mL) were added Pd(OAc)₂ (8.47 mg, 37.7 μmol, 0.0500 eq), Cs₂CO₃ (492 mg, 1.51 mmol, 2.00 eq), 1-(5-amino-3-pyridyl)pyrrolidin-2-one (294 mg, 830 μmol, 50.0% purity, 1.10 eq) and Xantphos (13.1 mg, 22.7 μmol, 0.0300 eq) at 20° C. under N₂ atmosphere. The mixture was stirred at 100° C. for 12 h. LCMS showed 2,5-dichloro-4-(6-methoxy-[1,1′-biphenyl]-3-yl)pyrimidine (8-5) was consumed completely and the desired mass was detected. The reaction mixture was concentrated under reduced pressure to remove solvent. The residue was purified by prep-HPLC (column: Waters Xbridge BEH C18 100*30 mm*10 um; mobile phase: [water (NH₄HCO₃)-ACN]; B %: 45%-75%, 8 min) to give the title compound (Compound 8) (27.4 mg, 56.7 μmol, 7.51% yield, 97.7% purity) as a white solid. LCMS (ES⁺): m/z 472.0 [M+H]⁺.

### Example 9. Synthesis of 1-(5-((5-chloro-4-(3-(pyridin-2-yl)phenyl)pyrimidin-2-yl)amino)pyridin-3-yl)pyrrolidin-2-one (Compound 9)

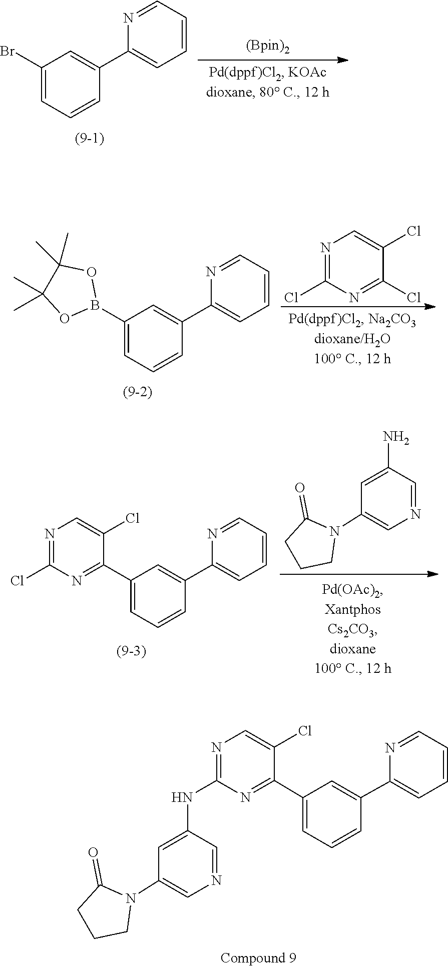

Synthesis of 2-(3-(4,4,5,5-tetramethyl-1,3,2-dioxaborolan-2-yl)phenyl)pyridine (9-2). To a solution of 2-(3-bromophenyl)pyridine (9-1) (500 mg, 2.14 mmol, 1.00 eq) in dioxane (5 mL) were added Pd(dppf)Cl₂ (156 mg, 214 μmol, 0.100 eq), KOAc (420 mg, 4.28 mmol, 2.00 eq) and 4,4,5,5-tetramethyl-2-(4,4,5,5-tetramethyl-1,3,2-dioxaborolan-2-yl)-1,3,2-dioxaborolane (815 mg, 3.21 mmol, 1.50 eq) at 25° C. under N₂. The mixture was stirred at 80° C. for 12 h under N₂ atmosphere. LCMS showed 2-(3-bromophenyl)pyridine (9-1) was consumed completely and the desired mass was detected. H₂O (15 mL) was added to the mixture and the aqueous was extracted with EtOAc (15 mL*3). The combined organic layers were dried over Na₂SO₄, filtered and the filtrate was concentrated under reduced pressure. The residue was purified by column chromatography (SiO₂, Petroleum ether:Ethyl acetate=1:0 to 3:1) to give the title compound (9-2) (600 mg, 2.13 mmol, 99.7% yield) as a white solid. LCMS (ES⁺): m/z 282.3 [M+H]⁺.

Synthesis of 2,5-dichloro-4-(3-(pyridin-2-yl)phenyl)pyrimidine (9-3). To a solution of 2-(3-(4,4,5,5-tetramethyl-1,3,2-dioxaborolan-2-yl)phenyl)pyridine (9-2) (560 mg, 1.99 mmol, 1.00 eq) in dioxane (1 mL) and H₂O (1 mL) were added Pd(dppf)Cl₂ (146 mg, 199 μmol, 0.100 eq), Na₂CO₃ (211 mg, 1.99 mmol, 1.00 eq) and 2,4,5-trichloropyrimidine (731 mg, 3.98 mmol, 2.00 eq) at 25° C. under N₂. The mixture was stirred at 100° C. for 12 h under N₂ atmosphere. LCMS showed 2-(3-(4,4,5,5-tetramethyl-1,3,2-dioxaborolan-2-yl)phenyl)pyridine (9-2) was consumed completely and the desired mass was detected. H₂O (10 mL) was added to the mixture and the aqueous phase was extracted with EtOAc (10 mL*3). The combined organic layers were dried over Na₂SO₄, filtered and the filtrate was concentrated under reduced pressure. The residue was purified by column chromatography (SiO₂, Petroleum ether:Ethyl acetate=1:0 to 3:1) to give the title compound (9-3) (400 mg, 1.32 mmol, 66.5% yield) as a black solid. LCMS (ES⁺): m/z 301.9 [M+H]⁺.

Synthesis of Compound 9. To a solution of 2,5-dichloro-4-(3-(pyridin-2-yl)phenyl)pyrimidine (9-3) (200 mg, 662 μmol, 1.00 eq) in dioxane (5 mL) were added Pd(OAc)₂ (14.9 mg, 66.2 μmol, 0.100 eq), Xantphos (38.3 mg, 66.2 μmol, 0.100 eq), Cs₂CO₃ (431 mg, 1.32 mmol, 2.00 eq) and 1-(5-amino-3-pyridyl)pyrrolidin-2-one (235 mg, 1.32 mmol, 2.00 eq) at 25° C. under N₂. The mixture was stirred at 100° C. for 12 h under N₂ atmosphere. LCMS showed 2,5-dichloro-4-(3-(pyridin-2-yl)phenyl)pyrimidine (9-3) was consumed completely and the desired mass was detected. H₂O (5 mL) was added to the mixture and the aqueous phase was extracted with EtOAc (5 mL*3). The combined organic layers were dried over Na₂SO₄, filtered and the filtrate was concentrated under reduced pressure. The residue was purified by prep-HPLC (column: Phenomenex Luna C18 75*30 mm*3 um; mobile phase: [water (FA)-ACN]; B %: 10%-50%, 8 min) to give the title compound (Compound 9) (74.1 mg, 167 μmol, 25.3% yield, 100% purity) as a white solid. LCMS (ES⁺): m/z 443.1 [M+H]⁺.

### Example 10. Synthesis of 1-(5-((5-chloro-4-(4-methyl-[1,1′-biphenyl]-3-yl)pyrimidin-2-yl)amino)pyridin-3-yl)pyrrolidin-2-one (Compound 10)

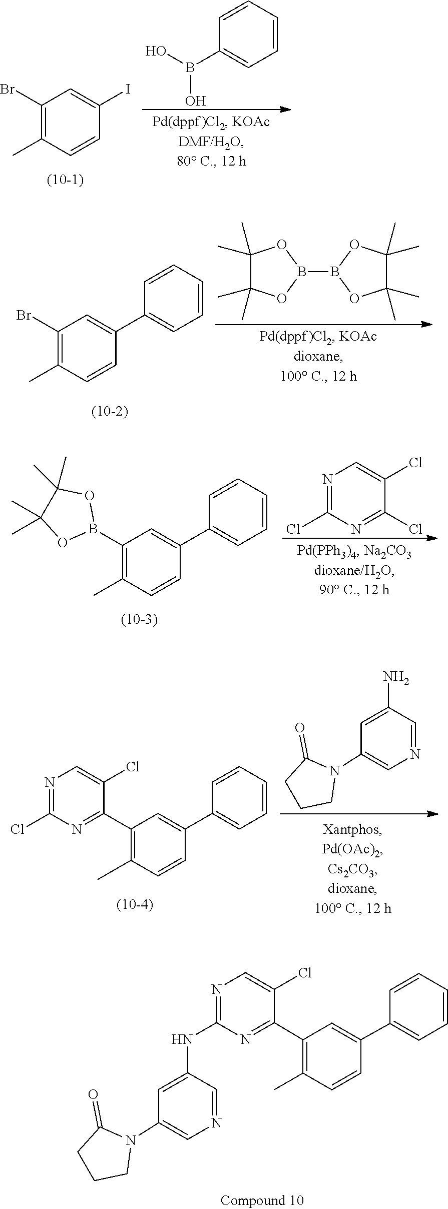

Synthesis of 3-bromo-4-methyl-1,1′-biphenyl (10-2). To a solution of 2-bromo-4-iodo-1-methylbenzene (10-1) (1.00 g, 3.37 mmol, 1.00 eq) and phenylboronic acid (411 mg, 3.37 mmol, 1.00 eq) in DMF (10 mL) and H₂O (1 mL) were added Pd(dppf)Cl₂ (246 mg, 337 μmol, 0.100 eq) and AcOK (661 mg, 6.74 mmol, 2.00 eq) at 20° C., then the solution was stirred at 80° C. for 12 h. TLC (SiO₂, Petroleum ether:Ethyl acetate=1:0) showed the 2-bromo-4-iodo-1-methylbenzene (10-1) was consumed, new spot was detected. Then H₂O (10 mL) was added to the solution, then the solution was extracted with EtOAc (20 mL*3), the organic layers was dried with Na₂SO₄, then concentrated. The residue was purified by MPLC (SiO₂, Petroleum ether:Ethyl acetate=1:0 to 0:1) to give the title compound (10-2) (520 mg, 2.10 mmol, 62.5% yield) as a white solid.

Synthesis of 4,4,5,5-tetramethyl-2-(4-methyl-[1,1′-biphenyl]-3-yl)-1,3,2-dioxaborolane (10-3). To a solution of 3-bromo-4-methyl-1,1′-biphenyl (10-2) (520 mg, 2.10 mmol, 1.00 eq) and 4,4,5,5-tetramethyl-2-(4,4,5,5-tetramethyl-1,3,2-dioxaborolan-2-yl)-1,3,2-dioxaborolane (641 mg, 2.52 mmol, 1.20 eq) in dioxane (10 mL) were added Pd(dppf)Cl₂ (77.0 mg, 105 μmol, 0.0500 eq) and KOAc (413 mg, 4.21 mmol, 2.00 eq) at 20° C., then the solution was stirred at 100° C. for 12 h. TLC (SiO₂, Petroleum ether:Ethyl acetate=5:1) showed 3-bromo-4-methyl-1,1′-biphenyl (10-2) was consumed, new spot formed. Then H₂O (20 mL) was added to the solution, then the solution was extracted with EtOAc (30 mL*3), the organic layers was dried with Na₂SO₄, then concentrated. The residue was purified by MPLC (SiO₂, Petroleum ether:Ethyl acetate=1:0 to 0:1) to give the title compound (10-3) (400 mg, 1.36 mmol, 64.6% yield) as a white solid.

Synthesis of 2,5-dichloro-4-(4-methyl-[1,1′-biphenyl]-3-yl)pyrimidine (10-4). To a solution of 4,4,5,5-tetramethyl-2-(4-methyl-[1,1′-biphenyl]-3-yl)-1,3,2-dioxaborolane (10-3) (300 mg, 1.02 mmol, 1.00 eq) in dioxane (10 mL) were added 2,4,5-trichloropyrimidine (374 mg, 2.04 mmol, 2.00 eq), Pd(PPh₃)₄ (23.6 mg, 20.4 μmol, 0.0200 eq), Na₂CO₃ (216 mg, 2.04 mmol, 2.00 eq) and H₂O (1 mL) at 25° C. under N₂ atmosphere. The mixture was stirred at 90° C. for 12 h. LCMS showed 4,4,5,5-tetramethyl-2-(4-methyl-[1,1′-biphenyl]-3-yl)-1,3,2-dioxaborolane (10-3) was consumed completely and the desired mass was detected. The residue was purified by prep-TLC (SiO₂, Petroleum ether:Ethyl acetate=10:1) to give the title compound (10-4) (134 mg, 424 μmol, 41.6% yield) as a white solid. LCMS (ES⁺): m/z 315.1 [M+H]⁺.

Synthesis of 1-(5-((5-chloro-4-(4-methyl-[1,1′-biphenyl]-3-yl)pyrimidin-2-yl)amino)pyridin-3-yl)pyrrolidin-2-one (Compound 10). To a solution of 2,5-dichloro-4-(4-methyl-[1,1′-biphenyl]-3-yl)pyrimidine (10-4) (134 mg, 424 μmol, 0.800 eq) in dioxane (5 mL) were added 1-(5-amino-3-pyridyl)pyrrolidin-2-one (144 mg, 583 μmol, 72.0% purity, 1.10 eq), Xantphos (9.20 mg, 15.9 μmol, 0.0300 eq), Cs₂CO₃ (346 mg, 1.06 mmol, 2.00 eq) and Pd(OAc)₂ (5.95 mg, 26.5 μmol, 0.0500 eq) at 25° C. under N₂ atmosphere. The mixture was stirred at 100° C. for 12 h. LCMS showed 2,5-dichloro-4-(4-methyl-[1,1′-biphenyl]-3-yl)pyrimidine (10-4) was consumed completely and the desired mass was detected. The residue was purified by prep-HPLC (column: Phenomenex luna C18 100*40 mm*3 um; mobile phase: [water (FA)-ACN]; B %: 30%-70%, 8 min) to give the title compound (Compound 10) (32.6 mg, 71.5 μmol, 13.5% yield, 100% purity) as a white solid. LCMS (ES⁺): m/z 456.1 [M+H]⁺.

### Example 11. Synthesis of 1-(5-((5-chloro-4-(5-methyl-[1,1′-biphenyl]-3-yl)pyrimidin-2-yl)amino)pyridin-3-yl)pyrrolidin-2-one (Compound 11)

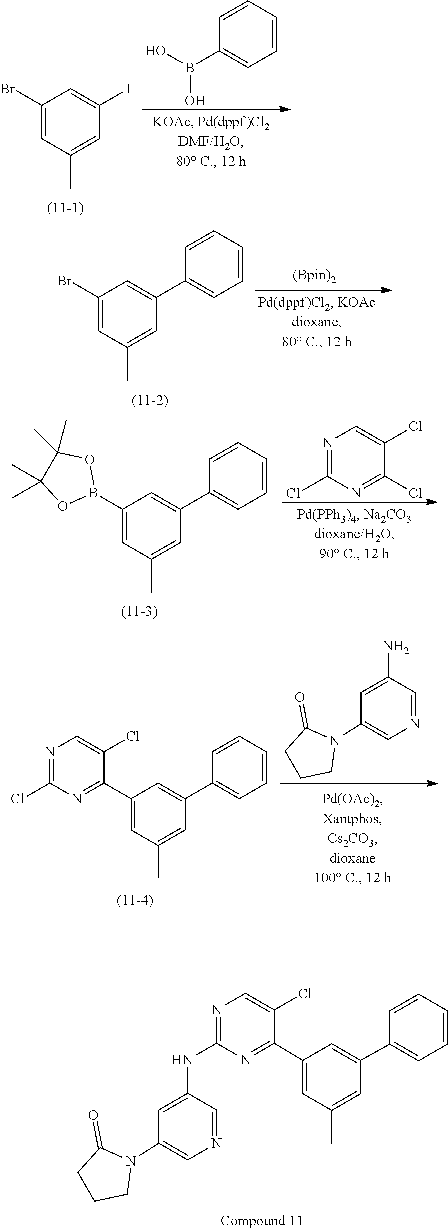

Synthesis of 3-bromo-5-methyl-1,1′-biphenyl (11-2). To a solution of 1-bromo-3-iodo-5-methylbenzene (11-1) (900 mg, 3.03 mmol, 1.00 eq) in DMF (12 mL) and H₂O (1.2 mL) were added KOAc (595 mg, 6.06 mmol, 2.00 eq), phenylboronic acid (554 mg, 4.55 mmol, 1.50 eq) and Pd(dppf)Cl₂ (222 mg, 303 μmol, 0.100 eq) at 20° C. under N₂ atmosphere. The mixture was stirred at 80° C. for 12 h. TLC (SiO₂, Ethyl acetate:Petroleum ether=0:1) indicated 1-bromo-3-iodo-5-methylbenzene (11-1) was consumed completely and one new spot formed. Water (12 mL) was added to the mixture at 20° C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:0 to 1:0) to give the title compound (11-2) (560 mg, 2.27 mmol, 74.8% yield) as a colorless oil.

Synthesis of 4,4,5,5-tetramethyl-2-(5-methyl-[1,1′-biphenyl]-3-yl)-1,3,2-dioxaborolane (11-3). To a solution of 3-bromo-5-methyl-1,1′-biphenyl (11-2) (560 mg, 2.27 mmol, 1.00 eq) in dioxane (10 mL) were added KOAc (445 mg, 4.53 mmol, 2.00 eq), 4,4,5,5-tetramethyl-2-(4,4,5,5-tetramethyl-1,3,2-dioxaborolan-2-yl)-1,3,2-dioxaborolane (691 mg, 2.72 mmol, 1.20 eq) and Pd(dppf)Cl₂ (82.9 mg, 113 μmol, 0.0500 eq) at 20° C. under N₂ atmosphere. The mixture was stirred at 80° C. for 12 h. LCMS showed 3-bromo-5-methyl-1,1′-biphenyl (11-2) was consumed completely and the desired mass was detected. The reaction mixture was concentrated under reduced pressure to remove solvent. The residue was purified by column chromatography (SiO₂, Petroleum ether:Ethyl acetate=1:0 to 20:1) to give the title compound (11-3) (580 mg, 1.97 mmol, 87.0% yield) as a colorless oil. LCMS (ES⁺): m/z 295.2 [M+H]⁺.

Synthesis of 2,5-dichloro-4-(5-methyl-[1,1′-biphenyl]-3-yl)pyrimidine (11-4). To a solution of 4,4,5,5-tetramethyl-2-(5-methyl-[1,1′-biphenyl]-3-yl)-1,3,2-dioxaborolane (11-3) (580 mg, 1.97 mmol, 1.00 eq) in dioxane (10 mL) and H₂O (1 mL) were added Pd(PPh₃)₄ (45.6 mg, 39.4 μmol, 0.0200 eq), 2,4,5-trichloropyrimidine (723 mg, 3.94 mmol, 2.00 eq) and Na₂CO₃ (418 mg, 3.94 mmol, 2.00 eq) at 20° C. under N₂ atmosphere. The mixture was stirred at 90° C. for 12 h. LCMS showed 4,4,5,5-tetramethyl-2-(5-methyl-[1,1′-biphenyl]-3-yl)-1,3,2-dioxaborolane (11-3) was consumed completely and the desired mass was detected. The reaction mixture was concentrated under reduced pressure to remove solvent. The residue was purified by prep-TLC (SiO₂, Petroleum ether:Ethyl acetate=10:1) to give the title compound (11-4) (416 mg, 1.32 mmol, 67.0% yield) as a colorless oil. LCMS (ES⁺): m/z 315.1 [M+H]⁺.

Synthesis of 1-(5-((5-chloro-4-(5-methyl-[1,1′-biphenyl]-3-yl)pyrimidin-2-yl)amino)pyridin-3-yl)pyrrolidin-2-one (Compound 11). To a solution of 2,5-dichloro-4-(5-methyl-[1,1′-biphenyl]-3-yl)pyrimidine (11-4) (200 mg, 635 μmol, 0.800 eq) in dioxane (6 mL) were added Pd(OAc)₂ (8.90 mg, 39.7 μmol, 0.0500 eq), 1-(5-amino-3-pyridyl)pyrrolidin-2-one (215 mg, 872 μmol, 72.0% purity, 1.10 eq), Cs₂CO₃ (517 mg, 1.59 mmol, 2.00 eq) and Xantphos (13.8 mg, 23.8 μmol, 0.0300 eq) at 20° C. under N₂ atmosphere. The mixture was stirred at 100° C. for 12 h. LCMS showed 2,5-dichloro-4-(5-methyl-[1,1′-biphenyl]-3-yl)pyrimidine (11-4) was consumed completely and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 40%-80%, 8 min) to give the title compound (Compound 11) (40.6 mg, 87.4 μmol, 11.0% yield, 98.1% purity) as a white solid. LCMS (ES⁺): m/z 456.1 [M+H]⁺.

### Example 12. Synthesis of 1-(5-((5-chloro-4-(3-cyclopropylphenyl)pyrimidin-2-yl)amino)pyridin-3-yl)pyrrolidin-2-one (Compound 12)

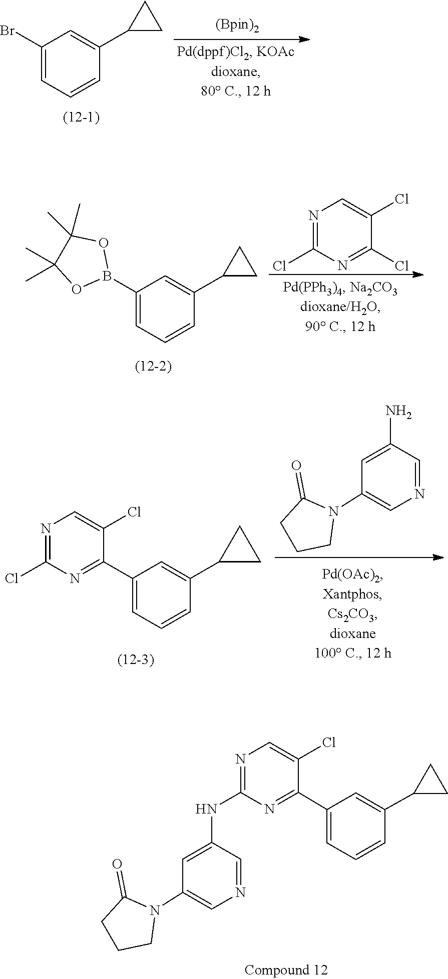

Synthesis of 2-(3-cyclopropylphenyl)-4,4,5,5-tetramethyl-1,3,2-dioxaborolane (12-2). To a solution of 1-bromo-3-cyclopropylbenzene (12-1) (300 mg, 1.52 mmol, 1.00 eq) in dioxane (10 mL) were added KOAc (299 mg, 3.04 mmol, 2.00 eq), 4,4,5,5-tetramethyl-2-(4,4,5,5-tetramethyl-1,3,2-dioxaborolan-2-yl)-1,3,2-dioxaborolane (464 mg, 1.83 mmol, 1.20 eq) and Pd(dppf)Cl₂ (55.7 mg, 76.1 μmol, 0.0500 eq) at 25° C. under N₂ atmosphere. The mixture was stirred at 80° C. for 12 h. LCMS showed 1-bromo-3-cyclopropylbenzene (12-1) was consumed completely and the desired mass was detected. The reaction mixture was concentrated under reduced pressure to remove solvent. The residue was purified by prep-TLC (SiO₂, Petroleum ether:Ethyl acetate=20:1) to give the title compound (12-2) (210 mg, 860 μmol, 56.5% yield) as a colorless oil. LCMS (ES⁺): m/z 245.1 [M+H]⁺.

Synthesis of 2,5-dichloro-4-(3-cyclopropylphenyl)pyrimidine (12-3). To a solution of 2-(3-cyclopropylphenyl)-4,4,5,5-tetramethyl-1,3,2-dioxaborolane (12-2) (210 mg, 860 μmol, 1.00 eq) in dioxane (6 mL) and H₂O (0.6 mL) were added Pd(PPh₃)₄ (19.9 mg, 17.2 μmol, 0.0200 eq), 2,4,5-trichloropyrimidine (316 mg, 1.72 mmol, 2.00 eq) and Na₂CO₃ (182 mg, 1.72 mmol, 2.00 eq) at 25° C. under N₂ atmosphere. The mixture was stirred at 90° C. for 12 h. LCMS showed 2-(3-cyclopropylphenyl)-4,4,5,5-tetramethyl-1,3,2-dioxaborolane (12-2) was consumed completely and the desired mass was detected. The reaction mixture was concentrated under reduced pressure to remove solvent. The residue was purified by prep-TLC (SiO₂, Petroleum ether Ethyl acetate=10:1) to give the title compound (12-3) (170 mg, 641 μmol, 74.5% yield) as a yellow oil. LCMS (ES⁺): m/z 265.0 [M+H]⁺.

Synthesis of 1-(5-((5-chloro-4-(3-cyclopropylphenyl)pyrimidin-2-yl)amino)pyridin-3-yl)pyrrolidin-2-one (Compound 12). To a solution of 1-(5-amino-3-pyridyl)pyrrolidin-2-one (203 mg, 882 μmol, 77.0% purity, 1.10 eq) in dioxane (6 mL) were added Pd(OAc)₂ (9.00 mg, 40.1 μmol, 0.0500 eq), 2,5-dichloro-4-(3-cyclopropylphenyl)pyrimidine (12-3) (170 mg, 641 μmol, 0.800 eq), Cs₂CO₃ (522 mg, 1.60 mmol, 2.00 eq) and Xantphos (13.9 mg, 24.0 μmol, 0.0300 eq) at 25° C. under N₂ atmosphere. The mixture was stirred at 100° C. for 12 h. LCMS showed 2,5-dichloro-4-(3-cyclopropylphenyl)pyrimidine (12-3) was consumed completely and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 45%-75%, 8 min) to give the title compound (Compound 12) (26.5 mg, 65.3 μmol, 8.15% yield, 100% purity) as a white solid. LCMS (ES⁺): m/z 406.1 [M+H]⁺.

### Example 13. Synthesis of 1-(5-((5-chloro-4-(4-cyclopropylphenyl)pyrimidin-2-yl)amino)pyridin-3-yl)pyrrolidin-2-one (Compound 13)

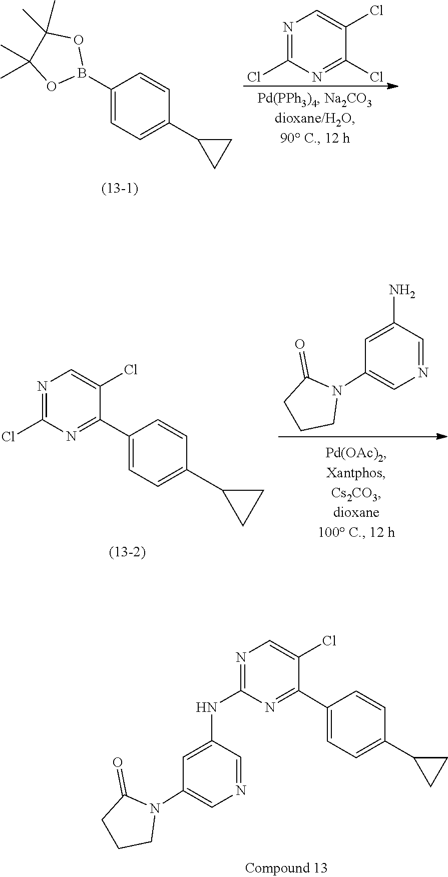 [SMILES: CC1(C)OB(c2ccc(C3CC3)cc2)OC1(C)C ; Clc1ncc(Cl)c(Cl)n1 ; Clc1ncc(Cl)c(-c2ccc(C3CC3)cc2)n1 ; Nc1cncc(N2CCCC2=O)c1 ; O=C1CCCN1c1cncc(Nc2ncc(Cl)c(-c3ccc(C4CC4)cc3)n2)c1]

Synthesis of 2,5-dichloro-4-(4-cyclopropylphenyl)pyrimidine (13-2). To a solution of 2,4,5-trichloropyrimidine (200 mg, 1.09 mmol, 1.00 eq) in dioxane (6 mL) and H₂O (0.6 mL) were added 2-(4-cyclopropylphenyl)-4,4,5,5-tetramethyl-1,3,2-dioxaborolane (13-1) (319 mg, 1.31 mmol, 1.20 eq), Pd(PPh₃)₄ (25.2 mg, 21.8 μmol, 0.0200 eq) and Na₂CO₃ (231 mg, 2.18 mmol, 2.00 eq) at 20° C. under N₂, the mixture was stirred at 90° C. for 12 h under N₂ atmosphere. LCMS showed 2-(4-cyclopropylphenyl)-4,4,5,5-tetramethyl-1,3,2-dioxaborolane (13-1) was consumed completely and the desired mass was detected. The reaction was concentrated under reduced pressure. MeOH (3 mL) was added to the mixture, then it was filtered and the filter cake was dried under reduced pressure to give the title compound (13-2) (120 mg, 453 μmol, 41.5% yield) as a yellow solid. LCMS (ES⁺): m/z 265.0 [M+H]⁺.

Synthesis of 1-(5-((5-chloro-4-(4-cyclopropylphenyl)pyrimidin-2-yl)amino)pyridin-3-yl)pyrrolidin-2-one (Compound 13). To a solution of 2,5-dichloro-4-(4-cyclopropylphenyl)pyrimidine (13-2) (115 mg, 434 μmol, 0.800 eq) in dioxane (4 mL) were added 1-(5-amino-3-pyridyl)pyrrolidin-2-one (147 mg, 596 μmol, 72.0% purity, 1.10 eq), Xantphos (9.41 mg, 16.3 μmol, 0.0300 eq), Cs₂CO₃ (353 mg, 1.08 mmol, 2.00 eq) and Pd(OAc)₂ (6.09 mg, 27.1 μmol, 0.0500 eq) at 20° C. under N₂, the mixture was stirred at 100° C. for 12 h under N₂ atmosphere. LCMS showed 2,5-dichloro-4-(4-cyclopropylphenyl)pyrimidine (13-2) was consumed completely and the desired mass was detected. The reaction was filtered and the filtrate was concentrated under reduced pressure. The residue was purified by prep-HPLC (column: Phenomenex luna C18 100*40 mm*3 um; mobile phase: [water (TFA)-ACN]; B %: 15%-60%, 8 min) to give the title compound (Compound 13) (50.0 mg, 123 μmol, 22.7% yield, 100% purity) as a light yellow solid. LCMS (ES⁺): m/z 406.1 [M+H]⁺.

### Example 14. Synthesis of 1-(5-((5-chloro-4-(2′-chloro-[1,1′-biphenyl]-3-yl)pyrimidin-2-yl)amino)pyridin-3-yl)pyrrolidin-2-one (Compound 14)

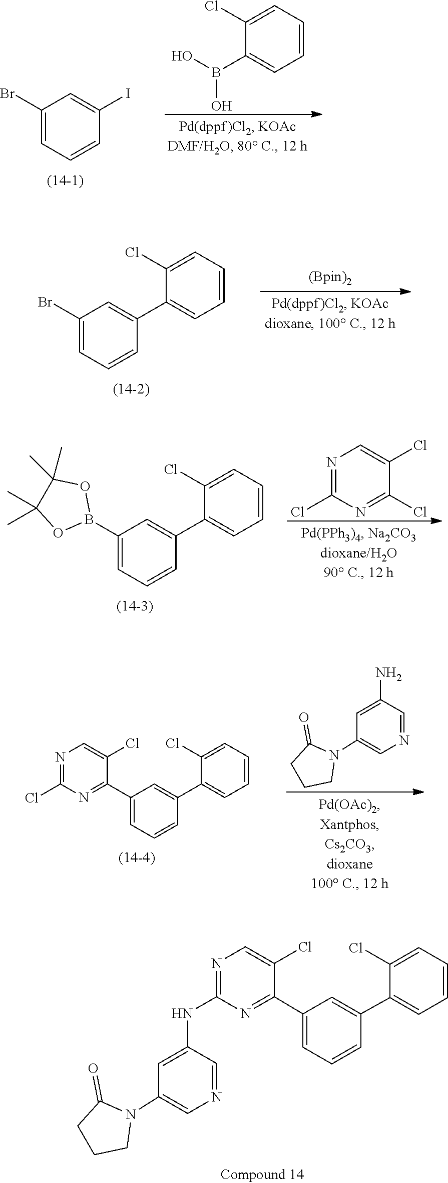

Synthesis of 3′-bromo-2-chloro-1,1′-biphenyl (14-2). To a solution of 1-bromo-3-iodobenzene (14-1) (500 mg, 1.77 mmol, 225 μL, 1.00 eq) and (2-chlorophenyl)boronic acid (276 mg, 1.77 mmol, 1.00 eq) in DMF (10 mL) and H₂O (1 mL) were added Pd(dppf)Cl₂ (129 mg, 177 μmol, 0.100 eq) and KOAc (347 mg, 3.53 mmol, 2.00 eq) at 20° C., then the solution was stirred at 80° C. for 12 h under N₂. TLC (Petroleum ether:Ethyl acetate=1:0) showed 1-bromo-3-iodobenzene (14-1) was consumed, new spot was detected. To the mixture was added H₂O (10 mL) and the mixture was extracted with EtOAc (10 mL*3). The combined organic layers were dried over Na₂SO₄ and then concentrated in vacuo. The mixture was purified by prep-TLC (SiO₂, PE:EtOAc=1:0) to give the title compound (14-2) (300 mg, 1.12 mmol, 63.4% yield) as colorless oil.

Synthesis of 2-(2′-chloro-[1,1′-biphenyl]-3-yl)-4,4,5,5-tetramethyl-1,3,2-dioxaborolane (14-3). To a solution of 3′-bromo-2-chloro-1,1′-biphenyl (14-2) (1.50 g, 5.61 mmol, 1.00 eq) and 4,4,5,5-tetramethyl-2-(4,4,5,5-tetramethyl-1,3,2-dioxaborolan-2-yl)-1,3,2-dioxaborolane (1.42 g, 5.61 mmol, 1.00 eq) in dioxane (5 mL) were added KOAc (1.10 g, 11.2 mmol, 2.00 eq) and Pd(dppf)Cl₂ (205 mg, 280 μmol, 0.0500 eq) at 20° C., then the mixture was stirred at 100° C. for 12 h under N₂. TLC (Petroleum ether:Ethyl acetate=5:1) showed 3′-bromo-2-chloro-1,1′-biphenyl (14-2) was consumed, new spot was detected. To the mixture was added H₂O (20 mL) and the mixture was extracted with EtOAc (10 mL*3). The combined organic layers were dried over Na₂SO₄ and then concentrated in vacuo to give the crude product. The mixture was purified by prep-TLC (SiO₂, Petroleum ether:Ethyl acetate=5:1) to give the title compound (14-3) (1.25 g, 3.97 mmol, 70.9% yield) as colorless oil.

Synthesis of 2,5-dichloro-4-(2′-chloro-[1,1′-biphenyl]-3-yl)pyrimidine (14-4). To a solution of 2-(2′-chloro-[1,1′-biphenyl]-3-yl)-4,4,5,5-tetramethyl-1,3,2-dioxaborolane (14-3) (1.20 g, 3.81 mmol, 1.00 eq) and 2,4,5-trichloropyrimidine (1.40 g, 7.63 mmol, 2.00 eq) in dioxane (20 mL) and H₂O (1 mL) were added Pd(PPh₃)₄ (88.2 mg, 76.3 μmol, 0.0200 eq) and Na₂CO₃ (809 mg, 7.63 mmol, 2.00 eq) at 20° C. under N₂ atmosphere. The mixture was stirred at 90° C. for 12 h. LCMS showed that 2-(2′-chloro-[1,1′-biphenyl]-3-yl)-4,4,5,5-tetramethyl-1,3,2-dioxaborolane (14-3) was consumed and the desired mass was detected. The reaction mixture was concentrated under reduced pressure to remove solvent. The mixture was purified by prep-TLC (SiO₂, PE:EtOAc=4:1) to give the title compound (14-4) (800 mg, 2.38 mmol, 62.5% yield) as colorless oil. LCMS (ES⁺): m/z 335.0 [M+H]⁺.

Synthesis of 1-(5-((5-chloro-4-(2′-chloro-[1,1′-biphenyl]-3-yl)pyrimidin-2-yl)amino)pyridin-3-yl)pyrrolidin-2-one (Compound 14). To a solution of 2,5-dichloro-4-(2′-chloro-[1,1′-biphenyl]-3-yl)pyrimidine (14-4) (200 mg, 596 μmol, 0.800 eq) and 1-(5-amino-3-pyridyl)pyrrolidin-2-one (201 mg, 819 μmol, 72.0% purity, 1.10 eq) in dioxane (3 mL) were added Pd(OAc)₂ (16.7 mg, 74.5 μmol, 0.100 eq), Cs₂CO₃ (485 mg, 1.49 mmol, 2.00 eq) and Xantphos (43.1 mg, 74.5 μmol, 0.100 eq) at 20° C., then the solution was stirred at 100° C. for 12 h under N₂. The mixture was concentrated in vacuo. The mixture was purified by prep-HPLC (column: Phenomenex Luna C18 75*30 mm*3 um; mobile phase: [water (FA)-ACN]; B %: 30%-70%, 8 min) to give the title compound (Compound 14) (48.7 mg, 95.8 μmol, 12.9% yield, 93.7% purity) as white solid. LCMS (ES⁺): m/z 476.1 [M+H]⁺.

### Example 15. Synthesis of 1-(5-((5-chloro-4-(2′-methyl-[1,1′-biphenyl]-3-yl)pyrimidin-2-yl)amino)pyridin-3-yl)pyrrolidin-2-one (Compound 15)

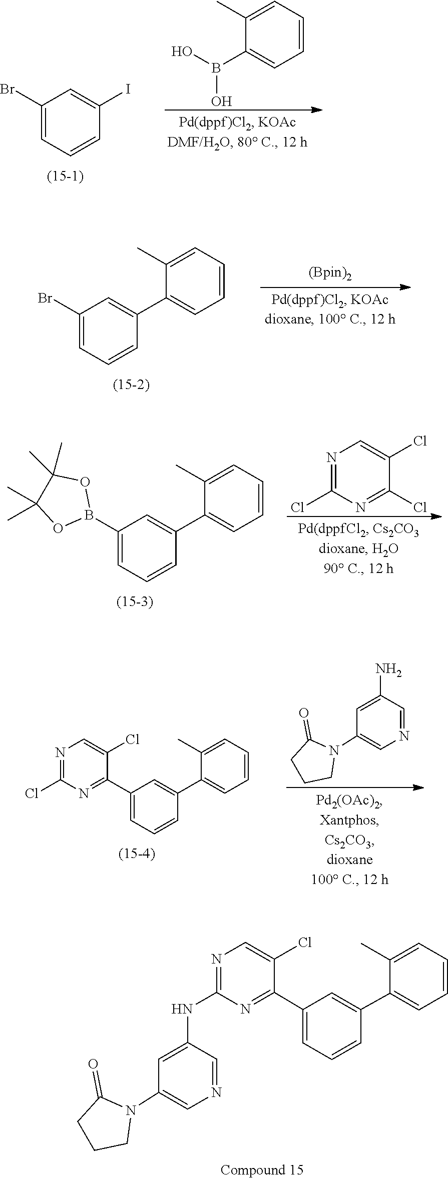

Synthesis of 3′-bromo-2-methyl-1,1′-biphenyl (15-2). To a solution of 1-bromo-3-iodobenzene (15-1) (1.00 g, 3.53 mmol, 450 μL, 1.00 eq) in DMF (10 mL) and H₂O (1 mL) were added o-tolylboronic acid (480 mg, 3.53 mmol, 1.00 eq), Pd(dppf)Cl₂ (258 mg, 353 μmol, 0.100 eq) and KOAc (693 mg, 7.06 mmol, 2.00 eq) at 20° C., then the solution was stirred at 80° C. for 12 h under N₂. TLC (SiO₂, Ethyl acetate:Petroleum ether=0:1) showed 1-bromo-3-iodobenzene (15-1) was consumed, a new spot was detected. To the mixture was added H₂O (10 mL) and the mixture was extracted with EtOAc (10 mL*3). The combined organic layers were dried over Na₂SO₄ and then concentrated in vacuo. The residue was purified by MPLC (SiO₂, Petroleum ether:Ethyl acetate=1:0 to 0:1) to give the title compound (15-2) (800 mg, 3.24 mmol, 91.7% yield) as a colorless oil.

Synthesis of 4,4,5,5-tetramethyl-2-(2′-methyl-[1,1′-biphenyl]-3-yl)-1,3,2-dioxaborolane (15-3). To a solution of 3′-bromo-2-methyl-1,1′-biphenyl (15-2) (800 mg, 3.24 mmol, 1.00 eq) in dioxane (20 mL) were added 4,4,5,5-tetramethyl-2-(4,4,5,5-tetramethyl-1,3,2-dioxaborolan-2-yl)-1,3,2-dioxaborolane (1.23 g, 4.86 mmol, 1.50 eq), Pd(dppf)Cl₂ (237 mg, 324 μmol, 0.100 eq) and KOAc (635 mg, 6.47 mmol, 2.00 eq) at 20° C., then the solution was stirred at 100° C. for 12 h under N₂. TLC (Petroleum ether:Ethyl acetate=5:1) showed 3′-bromo-2-methyl-1,1′-biphenyl (15-2) was consumed, new spot was detected. To the mixture was added H₂O (20 mL) and the mixture was extracted with EtOAc (20 mL*3). The combined organic layers were dried over Na₂SO₄ and then concentrated in vacuo to give the crude product. The mixture was purified by prep-TLC (SiO₂, PE:EtOAc=5:1) to give the title compound (15-3) (800 mg, 2.72 mmol, 84.0% yield) as a yellow solid.

Synthesis of 2,5-dichloro-4-(2′-methyl-[1,1′-biphenyl]-3-yl)pyrimidine (15-4). To a solution of 4,4,5,5-tetramethyl-2-(2′-methyl-[1,1′-biphenyl]-3-yl)-1,3,2-dioxaborolane (15-3) (770 mg, 2.62 mmol, 1.00 eq) in dioxane (15 mL) were added 2,4,5-trichloropyrimidine (1.44 g, 7.85 mmol, 3.00 eq), Pd(dppf)Cl₂ (192 mg, 262 μmol, 0.100 eq), Cs₂CO₃ (1.71 g, 5.23 mmol, 2.00 eq) and H₂O (1.5 mL) at 20° C., then the solution was stirred at 90° C. for 12 h under N₂. LCMS showed 4,4,5,5-tetramethyl-2-(2′-methyl-[1,1′-biphenyl]-3-yl)-1,3,2-dioxaborolane (15-3) was consumed completely and desired mass was detected. H₂O (20 mL) was added to the mixture, then the mixture was extracted with EtOAc (20 mL*3), then the combined organic layers were dried over Na₂SO₄, filtered and filtrate was concentrated to give a residue. The residue was purified by prep-TLC (SiO₂, Petroleum ether:Ethyl acetate=5:1) to give the title compound (15-4) (800 mg, 2.54 mmol, 97.0% yield) as a yellow solid. LCMS (ES⁺): m/z 315.0 [M+H]⁺.

Synthesis of 1-(5-((5-chloro-4-(2′-methyl-[1,1′-biphenyl]-3-yl)pyrimidin-2-yl)amino)pyridin-3-yl)pyrrolidin-2-one (Compound 15). To a solution of 2,5-dichloro-4-(2′-methyl-[1,1′-biphenyl]-3-yl)pyrimidine (15-4) (200 mg, 635 μmol, 0.800 eq) and 1-(5-aminopyridin-3-yl)pyrrolidin-2-one (215 mg, 872 μmol, 72.0% purity, 1.10 eq) in dioxane (2 mL) were added Pd(OAc)₂ (17.8 mg, 79.3 μmol, 0.100 eq), Cs₂CO₃ (517 mg, 1.59 mmol, 2.00 eq) and Xantphos (45.9 mg, 79.3 μmol, 0.100 eq) at 20° C., then the solution was stirred at 100° C. for 12 h under N₂. LCMS showed 2,5-dichloro-4-(2′-methyl-[1,1′-biphenyl]-3-yl)pyrimidine (15-4) was consumed completely and desired mass was detected. The mixture was concentrated in vacuo. The mixture was purified by prep-HPLC (column: Phenomenex luna C18 100*40 mm*3 um; mobile phase: [water (FA)-ACN]; B %: 30%-70%, 8 min) to give the title compound (Compound 15) (97.6 mg, 213 μmol, 26.9% yield, 99.6% purity) as white solid. LCMS (ES⁺): m/z 456.0 [M+H]⁺.

### Example 16. Synthesis of 1-(5-((5-chloro-4-(3-(pyridin-3-yl)phenyl)pyrimidin-2-yl)amino)pyridin-3-yl)pyrrolidin-2-one (Compound 16)

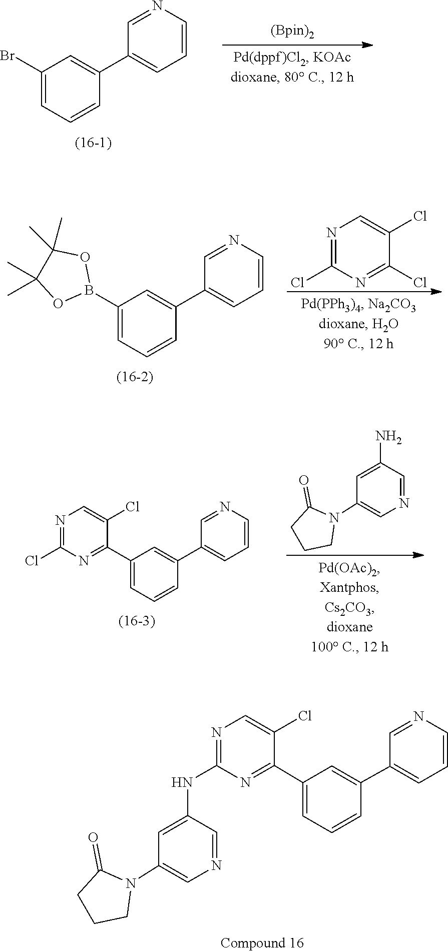

Synthesis of 3-(3-(4,4,5,5-tetramethyl-1,3,2-dioxaborolan-2-yl)phenyl)pyridine (16-2). To a solution of 3-(3-bromophenyl)pyridine (16-1) (600 mg, 2.56 mmol, 1.00 eq) and 4,4,5,5-tetramethyl-2-(4,4,5,5-tetramethyl-1,3,2-dioxaborolan-2-yl)-1,3,2-dioxaborolane (1.30 g, 5.13 mmol, 2.00 eq) in dioxane (7 mL) were added KOAc (503 mg, 5.13 mmol, 2.00 eq) and Pd(dppf)Cl₂ (188 mg, 256 μmol, 0.100 eq) at 20° C. under N₂. The mixture was stirred at 80° C. for 12 h. LCMS showed 3-(3-bromophenyl)pyridine (16-1) was consumed, desired target MS was detected. H₂O (10 mL) was added to the solution, then the mixture was extracted with EtOAc (10 mL*3), the combined organic layers were dried over Na₂SO₄, then concentrated in vacuo. The mixture was purified by prep-TLC (SiO₂, PE:EtOAc=1:1) to give the title compound (16-2) (500 mg, crude) as a yellow oil. LCMS (ES⁺): m/z 282.0 [M+H]⁺.

Synthesis of 2,5-dichloro-4-(3-(pyridin-3-yl)phenyl)pyrimidine (16-3). To a solution of 3-(3-(4,4,5,5-tetramethyl-1,3,2-dioxaborolan-2-yl)phenyl)pyridine (16-2) (300 mg, 1.07 mmol, 1.00 eq) and 2,4,5-trichloropyrimidine (391 mg, 2.13 mmol, 2.00 eq) in dioxane (7 mL) and H₂O (0.7 mL) were added Na₂CO₃ (226 mg, 2.13 mmol, 2.00 eq) and Pd(PPh₃)₄ (24.7 mg, 21.3 μmol, 0.0200 eq) at 20° C. The mixture was stirred at 90° C. for 12 h under N₂. LCMS showed 3-(3-(4,4,5,5-tetramethyl-1,3,2-dioxaborolan-2-yl)phenyl)pyridine (16-2) was consumed, desired target MS was detected. H₂O (5 mL) was added to the solution, then the mixture was extracted with EtOAc (5 mL*3), the combined organic layers were dried over Na₂SO₄, then concentrated in vacuo. The mixture was purified by prep-TLC (SiO₂, PE:EtOAc=3:1) to give the title compound (16-3) (225 mg, crude) as a yellow oil. LCMS (ES⁺): m/z 301.9 [M+H]⁺.

Synthesis of 1-(5-((5-chloro-4-(3-(pyridin-3-yl)phenyl)pyrimidin-2-yl)amino)pyridin-3-yl)pyrrolidin-2-one (Compound 16). To a stirred solution of 1-(5-aminopyridin-3-yl)pyrrolidin-2-one (179 mg, 728 μmol, 72.0% purity, 1.10 eq) and 2,5-dichloro-4-(3-(pyridin-3-yl)phenyl)pyrimidine (16-3) (200 mg, 662 μmol, 1.00 eq) in dioxane (4 mL) were added Cs₂CO₃ (431 mg, 1.32 mmol, 2.00 eq), Xantphos (38.3 mg, 66.2 μmol, 0.100 eq) and Pd(OAc)₂ (14.9 mg, 66.2 μmol, 0.100 eq) at 20° C. The resulting mixture was stirred at 100° C. for 12 h under N₂. LCMS showed 2,5-dichloro-4-(3-(pyridin-3-yl)phenyl)pyrimidine (16-3) was consumed, desired target MS was detected. H₂O (5 mL) was added to the solution, then the mixture was extracted with EtOAc (5 mL*3), the combined organic layers were dried over Na₂SO₄, then concentrated in vacuo. The residue was purified by prep-HPLC (column: Phenomenex Luna 80*30 mm*3 um; mobile phase: [water (FA)-ACN]; B %: 5%-45%, 8 min) to give the title compound (Compound 16) (48.2 mg, 100 μmol, 15.2% yield, 92.2% purity) as a white solid. LCMS (ES⁺): m/z 443.1 [M+H]⁺.

### Example 17. Synthesis of 1-(5-((5-chloro-4-(3-(pyrazin-2-yl)phenyl)pyrimidin-2-yl)amino)pyridin-3-yl)pyrrolidin-2-one (Compound 17)

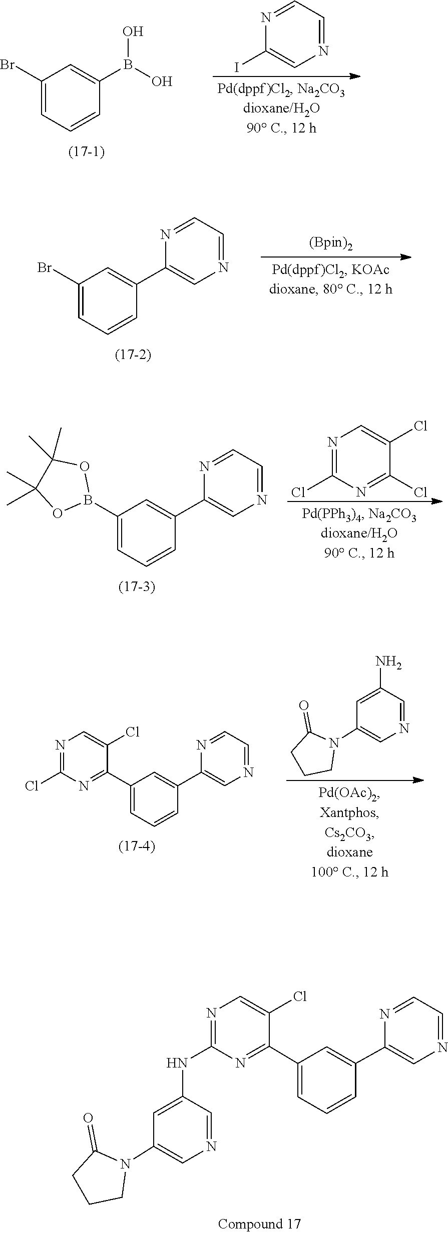

Synthesis of 2-(3-bromophenyl)pyrazine (17-2). To a solution of (3-bromophenyl)boronic acid (17-1) (2.92 g, 14.6 mmol, 1.50 eq) and 2-iodopyrazine (2.00 g, 9.71 mmol, 957 μL, 1.00 eq) in H₂O (3 mL) and dioxane (30 mL) were added Pd(dppf)Cl₂ (710 mg, 971 μmol, 0.100 eq) and Na₂CO₃ (2.06 g, 19.4 mmol, 2.00 eq) at 20° C. The mixture was stirred at 90° C. for 12 h under N₂. LCMS showed the reaction was complete, (3-bromophenyl)boronic acid (17-1) was consumed, desired target MS was detected. H₂O (30 mL) was added to the mixture and the aqueous was extracted with EtOAc (20 mL*3), the combined organic layers were dried over Na₂SO₄ and then concentrated in vacuo. The residue was purified by MPLC (SiO₂, PE:EtOAc=1:0 to 5:1) to give the title compound (17-2) (600 mg, crude) as a yellow solid. LCMS (ES⁺): m/z 235.0 [M+H]⁺.

Synthesis of 2-(3-(4,4,5,5-tetramethyl-1,3,2-dioxaborolan-2-yl)phenyl)pyrazine (17-3). To a solution of 2-(3-bromophenyl)pyrazine (17-2) (500 mg, 2.13 mmol, 1.00 eq) and 4,4,5,5-tetramethyl-2-(4,4,5,5-tetramethyl-1,3,2-dioxaborolan-2-yl)-1,3,2-dioxaborolane (1.08 g, 4.25 mmol, 2.00 eq) in dioxane (16 mL) were added KOAc (417 mg, 4.25 mmol, 2.00 eq) and Pd(dppf)Cl₂ (156 mg, 213 μmol, 0.100 eq) at 20° C. under N₂. The mixture was stirred at 80° C. for 12 h. LCMS showed 2-(3-bromophenyl)pyrazine (17-2) was consumed, desired target MS was detected. H₂O (15 mL) was added to the solution, then the mixture was extracted with EtOAc (10 mL*3), the combined organic layers were dried over Na₂SO₄, then concentrated in vacuo. The mixture was purified by MPLC (SiO₂, Petroleum ether:Ethyl acetate=1:0 to 1:1) to give the title compound (17-3) (680 mg, crude) as a yellow oil. LCMS (ES⁺): m/z 283.0 [M+H]⁺.

Synthesis of 2,5-dichloro-4-(3-(pyrazin-2-yl)phenyl)pyrimidine (17-4). To a solution of 2-(3-(4,4,5,5-tetramethyl-1,3,2-dioxaborolan-2-yl)phenyl)pyrazine (17-3) (330 mg, 1.17 mmol, 1.00 eq) and 2,4,5-trichloropyrimidine (429 mg, 2.34 mmol, 2.00 eq) in dioxane (10 mL) and H₂O (1 mL) were added Na₂CO₃ (248 mg, 2.34 mmol, 2.00 eq) and Pd(PPh₃)₄ (27.0 mg, 23.4 μmol, 0.0200 eq) at 20° C. Then the mixture was stirred at 90° C. for 12 h under N₂. LCMS showed 2-(3-(4,4,5,5-tetramethyl-1,3,2-dioxaborolan-2-yl)phenyl)pyrazine (17-3) was consumed, desired target MS was detected. H₂O (10 mL) was added to the solution, then the mixture was extracted with EtOAc (5 mL*3), the combined organic lays were dried over Na₂SO₄, then concentrated in vacuo. The mixture was purified by MPLC (SiO₂, Petroleum ether:Ethyl acetate=1:0 to 5:1) to give the title compound (17-4) (160 mg, 528 μmol, 45.1% yield) as a yellow oil. LCMS (ES⁺): m/z 303.0 [M+H]⁺.

Synthesis of 1-(5-((5-chloro-4-(3-(pyrazin-2-yl)phenyl)pyrimidin-2-yl)amino)pyridin-3-yl)pyrrolidin-2-one (Compound 17). To a stirred solution of 1-(5-aminopyridin-3-yl)pyrrolidin-2-one (125 mg, 508 μmol, 72.0% purity, 1.10 eq) and 2,5-dichloro-4-(3-(pyrazin-2-yl)phenyl)pyrimidine (17-4) (140 mg, 462 μmol, 1.00 eq) in dioxane (1 mL) were added Cs₂CO₃ (301 mg, 924 μmol, 2.00 eq), Xantphos (26.7 mg, 46.2 μmol, 0.100 eq) and Pd(OAc)₂ (10.4 mg, 46.2 μmol, 0.100 eq) at 20° C. The resulting mixture was stirred at 100° C. for 12 h. LCMS showed 2,5-dichloro-4-(3-(pyrazin-2-yl)phenyl)pyrimidine (17-4) was consumed, desired target MS was detected. H₂O (3 mL) was added to the solution, then the mixture was extracted with EtOAc (3 mL*3), the combined organic layers were dried over Na₂SO₄, then concentrated in vacuo. The residue was purified by prep-HPLC (column: Waters Xbridge BEH C18 100*30 mm*10 um; mobile phase: [water (NH₄HCO₃)-ACN]; B %: 25%-55%, 8 min) to give the title compound (Compound 17) (33.7 mg, 74.8 μmol, 16.2% yield, 98.6% purity) as a white solid. LCMS (ES⁺): m/z 444.1 [M+H]⁺.

### Example 18. Synthesis of 1-(5-((5-chloro-4-(5-methoxy-[1,1′-biphenyl]-3-yl)pyrimidin-2-yl)amino)pyridin-3-yl)pyrrolidin-2-one (Compound 18)

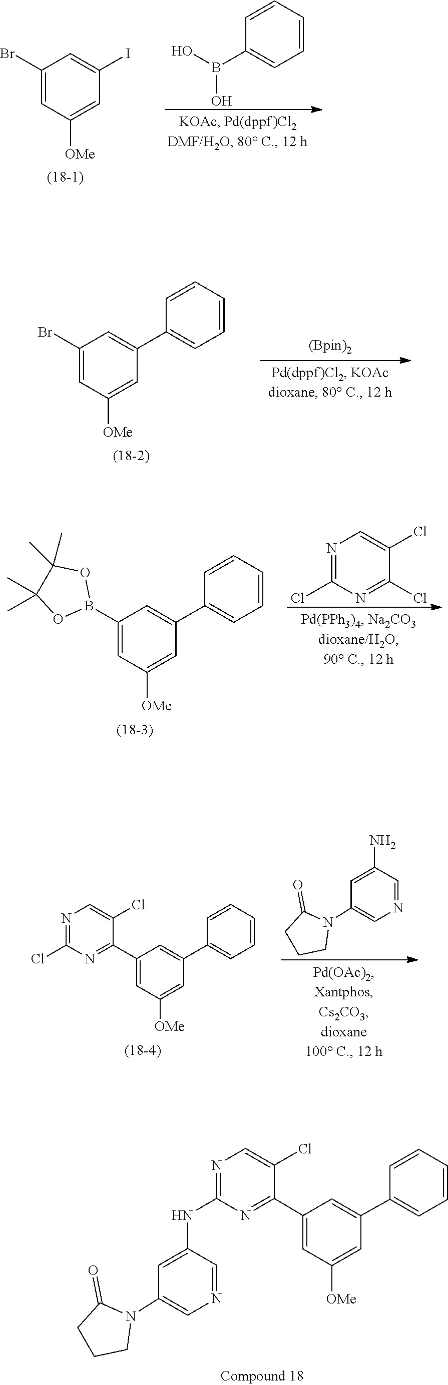

Synthesis of 3-bromo-5-methoxy-1,1′-biphenyl (18-2). To a solution of 1-bromo-3-iodo-5-methoxybenzene (18-1) (900 mg, 2.88 mmol, 1.00 eq) in DMF (12 mL) and H₂O (1.2 mL) were added KOAc (565 mg, 5.75 mmol, 2.00 eq), phenylboronic acid (526 mg, 4.31 mmol, 1.50 eq) and Pd(dppf)Cl₂ (210 mg, 288 μmol, 0.100 eq) at 20° C. under N₂ atmosphere. The mixture was stirred at 80° C. for 12 h. TLC (SiO₂, Ethyl acetate:Petroleum ether=0:1) indicated 1-bromo-3-iodo-5-methoxybenzene (18-1) was consumed completely and one new spot formed. Water (12 mL) was added to the mixture at 20° C. Then the reaction mixture was extracted with EtOAc (10 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:0 to 1:0) to give the title compound (18-2) (526 mg, 2.00 mmol, 69.5% yield) as a colorless oil.

Synthesis of 2-(5-methoxy-[1,1′-biphenyl]-3-yl)-4,4,5,5-tetramethyl-1,3,2-dioxaborolane (18-3). To a solution of 3-bromo-5-methoxy-1,1′-biphenyl (18-2) (525 mg, 2.00 mmol, 1.00 eq) in dioxane (10 mL) were added KOAc (392 mg, 3.99 mmol, 2.00 eq), 4,4,5,5-tetramethyl-2-(4,4,5,5-tetramethyl-1,3,2-dioxaborolan-2-yl)-1,3,2-dioxaborolane (608 mg, 2.39 mmol, 1.20 eq) and Pd(dppf)Cl₂ (73.0 mg, 99.8 μmol, 0.0500 eq) at 20° C. under N₂ atmosphere. The mixture was stirred at 80° C. for 12 h. LCMS showed 3-bromo-5-methoxy-1,1′-biphenyl (18-2) was consumed completely and the desired mass was detected. The reaction mixture was concentrated under reduced pressure to remove solvent. The residue was purified by prep-TLC (SiO₂, Petroleum ether:Ethyl acetate=20:1) to give the title compound (18-3) (500 mg, 1.61 mmol, 80.8% yield) as a colorless oil. LCMS (ES⁺): m/z 311.2 [M+H]⁺.

Synthesis of 2,5-dichloro-4-(5-methoxy-[1,1′-biphenyl]-3-yl)pyrimidine (18-4). To a solution of 2-(5-methoxy-[1,1′-biphenyl]-3-yl)-4,4,5,5-tetramethyl-1,3,2-dioxaborolane (18-3) (480 mg, 1.55 mmol, 1.00 eq) in dioxane (10 mL) and H₂O (1 mL) were added Pd(PPh₃)₄ (35.8 mg, 31.0 μmol, 0.0200 eq), 2,4,5-trichloropyrimidine (568 mg, 3.09 mmol, 2.00 eq) and Na₂CO₃ (328 mg, 3.09 mmol, 2.00 eq) at 20° C. under N₂ atmosphere. The mixture was stirred at 90° C. for 12 h. LCMS showed 2-(5-methoxy-[1,1′-biphenyl]-3-yl)-4,4,5,5-tetramethyl-1,3,2-dioxaborolane (18-3) was consumed completely and the desired mass was detected. The reaction mixture was concentrated under reduced pressure to remove solvent. The residue was purified by prep-TLC (SiO₂, Petroleum ether:Ethyl acetate=10:1) to give the title compound (18-4) (380 mg, 1.15 mmol, 74.2% yield) as a colorless oil. LCMS (ES⁺): m/z 331.1 [M+H]⁺.

Synthesis of 1-(5-((5-chloro-4-(5-methoxy-[1,1′-biphenyl]-3-yl)pyrimidin-2-yl)amino)pyridin-3-yl)pyrrolidin-2-one (Compound 18). To a solution of 2,5-dichloro-4-(5-methoxy-[1,1′-biphenyl]-3-yl)pyrimidine (18-4) (200 mg, 604 μmol, 0.800 eq) in dioxane (6 mL) were added Pd(OAc)₂ (8.47 mg, 37.7 μmol, 0.0500 eq), 1-(5-amino-3-pyridyl)pyrrolidin-2-one (204 mg, 830 μmol, 72.0% purity, 1.10 eq), Cs₂CO₃ (492 mg, 1.51 mmol, 2.00 eq) and Xantphos (13.1 mg, 22.7 μmol, 0.0300 eq) at 25° C. under N₂ atmosphere. The mixture was stirred at 100° C. for 12 h. LCMS showed 2,5-dichloro-4-(5-methoxy-[1,1′-biphenyl]-3-yl)pyrimidine (18-4) was consumed completely and the desired mass was detected. The reaction mixture was concentrated under reduced pressure to remove solvent. The residue was purified by prep-HPLC (column: Phenomenex Luna C18 75*30 mm*3 um; mobile phase: [water (FA)-MeOH]; B %: 50%-80%, 8 min) to give the title compound (Compound 18) (35.5 mg, 75.2 μmol, 9.97% yield, 100% purity) as a yellow solid. LCMS (ES⁺): m/z 472.1 [M+H]⁺.

### Example 19. Synthesis of 1-(5-((5-chloro-4-(3-cyclohexylphenyl) pyrimidin-2-yl)amino)pyridin-3-yl)pyrrolidin-2-one (Compound 19)

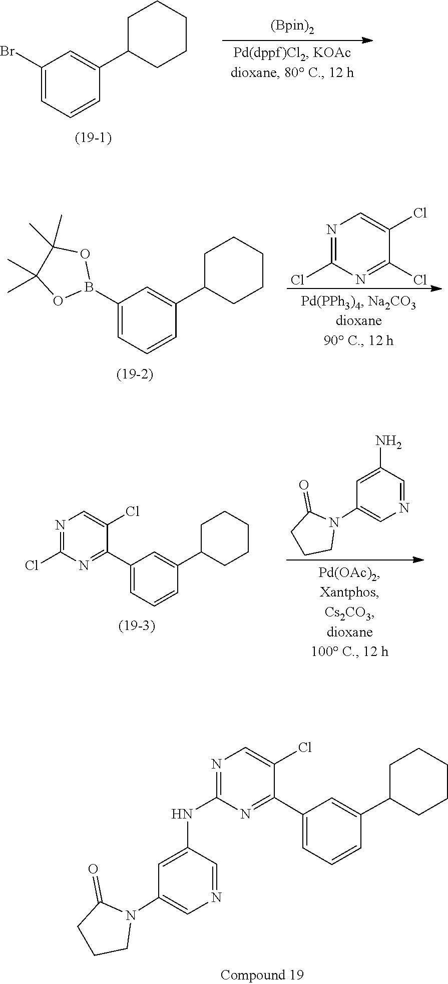

Synthesis of 2-(3-cyclohexylphenyl)-4,4,5,5-tetramethyl-1,3,2-dioxaborolane (19-2). To a stirred solution of 1-bromo-3-cyclohexylbenzene (19-1) (350 mg, 1.46 mmol, 1.00 eq) and 4,4,5,5-tetramethyl-2-(4,4,5,5-tetramethyl-1,3,2-dioxaborolan-2-yl)-1,3,2-dioxaborolane (743 mg, 2.93 mmol, 2.00 eq) in dioxane (11 mL) were added KOAc (287 mg, 2.93 mmol, 2.00 eq) and Pd(dppf)Cl₂ (107 mg, 146 μmol, 0.100 eq) at 20° C. The resulting mixture was stirred at 80° C. for 12 h under N₂. TLC (SiO₂, PE:EtOAc=5:1) showed 1-bromo-3-cyclohexylbenzene (19-1) was consumed and the desired mass was detected. The mixture was concentrated in vacuo. The mixture was purified by prep-TLC (SiO₂, PE:EtOAc=5:1) to give the title compound (19-2) (90.0 mg, 314 μmol, 21.5% yield) as a yellow oil.

Synthesis of 2,5-dichloro-4-(3-cyclohexylphenyl) pyrimidine (19-3). To a solution of 2-(3-cyclohexylphenyl)-4,4,5,5-tetramethyl-1,3,2-dioxaborolane (19-2) (90.0 mg, 314 μmol, 1.00 eq) and 2,4,5-trichloropyrimidine (115 mg, 629 μmol, 2.00 eq) in dioxane (2 mL) and H₂O (0.2 mL) were added Na₂CO₃ (66.7 mg, 629 μmol, 2.00 eq) and Pd(PPh₃)₄ (7.27 mg, 6.29 μmol, 0.0200 eq) at 20° C. Then the mixture was stirred at 90° C. for 12 h under N₂. LCMS showed 2-(3-cyclohexylphenyl)-4,4,5,5-tetramethyl-1,3,2-dioxaborolane (19-2) was consumed, desired target MS was detected. H₂O (3 mL) was added to the solution, then the mixture was extracted with EtOAc (3 mL*3), the combined organic layers were dried over Na₂SO₄, then concentrated in vacuo. The mixture was purified by prep-TLC (SiO₂, PE:EtOAc=5:1) to give the title compound (19-3) (70.0 mg, crude) as a yellow oil. LCMS (ES⁺): m/z 307.0 [M+H]⁺.

Synthesis of 1-(5-((5-chloro-4-(3-cyclohexylphenyl) pyrimidin-2-yl)amino)pyridin-3-yl)pyrrolidin-2-one (Compound 19). To a stirred solution of 1-(5-aminopyridin-3-yl)pyrrolidin-2-one (52.9 mg, 215 μmol, 72.0% purity, 1.10 eq) and 2,5-dichloro-4-(3-cyclohexylphenyl) pyrimidine (19-3) (60.0 mg, 195 μmol, 1.00 eq) in dioxane (3 mL) were added Cs₂CO₃ (127 mg, 391 μmol, 2.00 eq), Xantphos (11.3 mg, 19.5 μmol, 0.100 eq) and Pd(OAc)₂ (4.38 mg, 19.5 μmol, 0.100 eq) at 20° C. The resulting mixture was stirred at 100° C. for 12 h under N₂. LCMS showed 2,5-dichloro-4-(3-cyclohexylphenyl) pyrimidine (19-3) was consumed, desired target MS was detected. H₂O (3 mL) was added to the solution, then the mixture was extracted with EtOAc (4 mL*3), the combined organic layers were dried over Na₂SO₄, then concentrated in vacuo. The residue was purified by prep-HPLC (column: Phenomenex C18 75*30 mm*3 um; mobile phase: [water (NH₄HCO₃)-ACN]; B %: 45%-75%, 8 min) to give the title compound (Compound 19) (23.0 mg, 50.9 μmol, 26.1% yield, 99.2% purity) as a white solid. LCMS (ES⁺): m/z 448.2 [M+H]⁺.

### Example 20. Synthesis of 1-(5-((5-chloro-4-(4-chloro-[1,1′-biphenyl]-3-yl)pyrimidin-2-yl)amino)pyridin-3-yl)pyrrolidin-2-one (Compound 20)

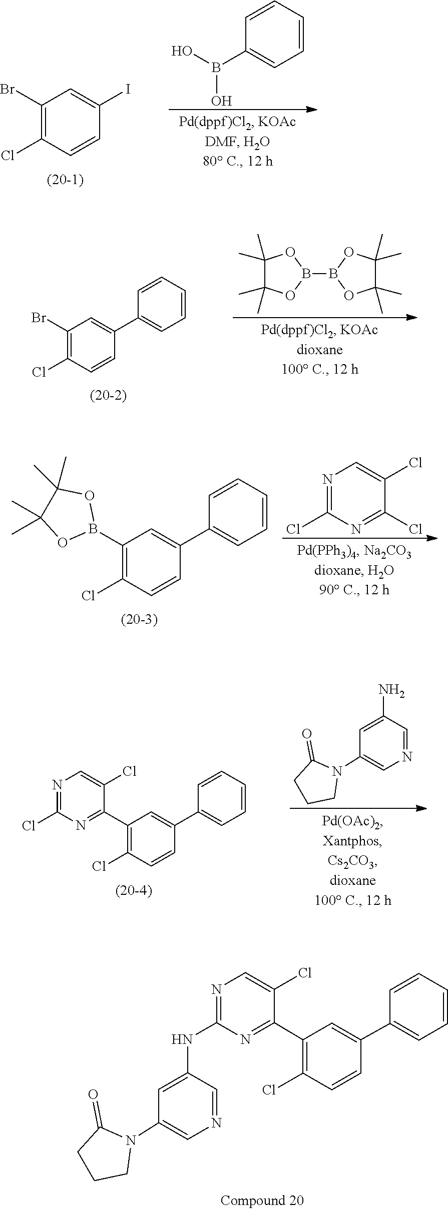

Synthesis of 3-bromo-4-chloro-1,1′-biphenyl (20-2). To a solution of 2-bromo-1-chloro-4-iodobenzene (20-1) (4.50 g, 14.2 mmol, 1.00 eq) in DMF (60 mL) and H₂O (6 mL) were added phenylboronic acid (2.07 g, 17.0 mmol, 1.20 eq), KOAc (2.78 g, 28.4 mmol, 2.00 eq), Pd(dppf)Cl₂ (1.04 g, 1.42 mmol, 0.100 eq) at 20° C. under N₂ atmosphere. The mixture was stirred at 80° C. for 12 h under N₂ atmosphere. LCMS showed 2-bromo-1-chloro-4-iodobenzene (20-1) was consumed completely and the desired mass was detected. The reaction mixture was concentrated under reduced pressure to remove solvent. The residue was purified by column chromatography (SiO₂, Petroleum ether:Ethyl acetate=1:0 to 1:0) to give the title compound (20-2) (2.65 g, 9.90 mmol, 69.9% yield) as a yellow solid.

Synthesis of 2-(4-chloro-[1,1′-biphenyl]-3-yl)-4,4,5,5-tetramethyl-1,3,2-dioxaborolane (20-3). To a solution of 3-bromo-4-chloro-1,1′-biphenyl (20-2) (2.65 g, 9.90 mmol, 1.00 eq) in dioxane (100 mL) were added 4,4,5,5-tetramethyl-2-(4,4,5,5-tetramethyl-1,3,2-dioxaborolan-2-yl)-1,3,2-dioxaborolane (3.77 g, 14.9 mmol, 1.50 eq), KOAc (1.94 g, 19.8 mmol, 2.00 eq) and Pd(dppf)Cl₂ (725 mg, 990 μmol, 0.100 eq) at 25° C. under N₂ atmosphere. The mixture was stirred at 100° C. for 12 h under N₂ atmosphere. TLC (SiO₂, Petroleum ether:Ethyl acetate=10:1) indicated 3-bromo-4-chloro-1,1′-biphenyl (20-2) was consumed completely and one new spots formed. The reaction was clean according to TLC. The reaction mixture was concentrated under reduced pressure to remove solvent. The residue was purified by column chromatography (SiO₂, Petroleum ether:Ethyl acetate=1:0 to 10:1) to give the title compound (20-3) (2.09 g, 6.64 mmol, 67.1% yield) as a yellow solid.

Synthesis of 2,5-dichloro-4-(4-chloro-[1,1′-biphenyl]-3-yl)pyrimidine (20-4). To a solution of 2-(4-chloro-[1,1′-biphenyl]-3-yl)-4,4,5,5-tetramethyl-1,3,2-dioxaborolane (20-3) (2.09 g, 6.64 mmol, 1.00 eq) in dioxane (90 mL) and H₂O (9 mL) were added 2,4,5-trichloropyrimidine (2.44 g, 13.3 mmol, 2.00 eq), Na₂CO₃ (1.41 g, 13.3 mmol, 2.00 eq) and Pd(PPh₃)₄ (154 mg, 133 μmol, 0.0200 eq) at 25° C. The mixture was stirred at 90° C. for 12 h under N₂ atmosphere. LCMS showed 2-(4-chloro-[1,1′-biphenyl]-3-yl)-4,4,5,5-tetramethyl-1,3,2-dioxaborolane (20-3) was consumed completely and the desired mass was detected. The reaction mixture was concentrated under reduced pressure to remove solvent. The residue was purified by column chromatography (SiO₂, Petroleum ether:Ethyl acetate=1:0 to 100:3) to give the title compound (20-4) (1.80 g, 5.36 mmol, 80.7% yield) as a yellow solid. LCMS (ES⁺): m/z 335.0 [M+H]⁺.

Synthesis of 1-(5-((5-chloro-4-(4-chloro-[1,1′-biphenyl]-3-yl)pyrimidin-2-yl)amino)pyridin-3-yl)pyrrolidin-2-one (Compound 20). To a solution of 2,5-dichloro-4-(4-chloro-[1,1′-biphenyl]-3-yl)pyrimidine (20-4) (200 mg, 596 μmol, 0.800 eq) in dioxane (3 mL) were added 1-(5-amino-3-pyridyl)pyrrolidin-2-one (85.7 mg, 372 μmol, 77.0% purity, 0.500 eq), Xantphos (12.9 mg, 22.4 μmol, 0.0300 eq), Cs₂CO₃ (485 mg, 1.49 mmol, 2.00 eq) and Pd(OAc)₂ (8.36 mg, 37.3 μmol, 0.0500 eq) at 25° C. under N₂ atmosphere. The mixture was stirred at 100° C. for 12 h under N₂ atmosphere. LCMS showed 2,5-dichloro-4-(4-chloro-[1,1′-biphenyl]-3-yl)pyrimidine (20-4) was consumed completely and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH₄HCO₃)-ACN]; B %: 50%-80%, 8 min) to give the title compound (Compound 20) (20.3 mg, 42.6 μmol, 5.72% yield, 100% purity) as a yellow solid. LCMS (ES⁺): m/z 476.0 [M+H]⁺.

### Example 21. Synthesis of 1-(5-((5-chloro-4-(4-methoxy-[1,1′-biphenyl]-3-yl)pyrimidin-2-yl)amino)pyridin-3-yl)pyrrolidin-2-one (Compound 21)

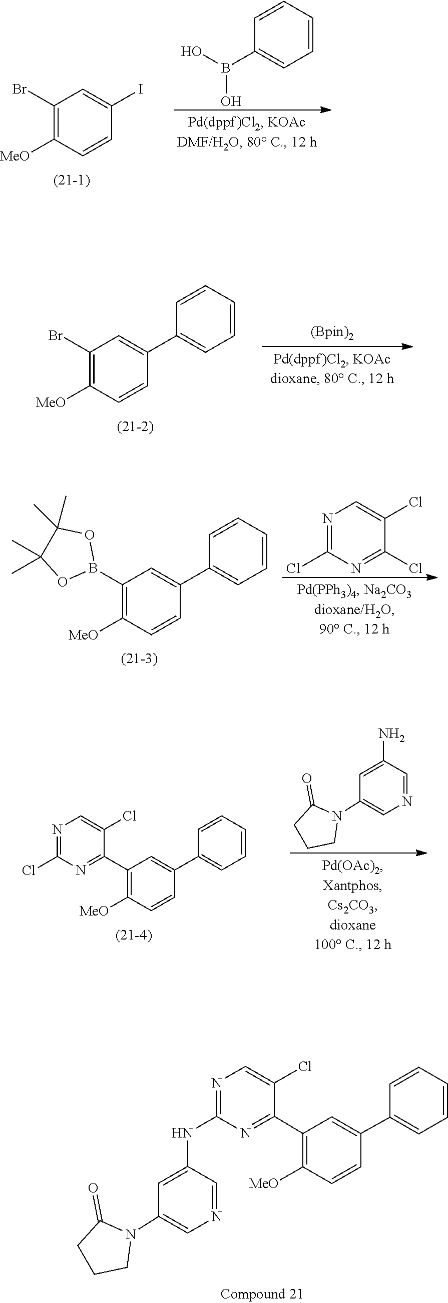

Synthesis of 3-bromo-4-methoxy-1,1′-biphenyl (21-2). To a solution of 2-bromo-4-iodo-1-methoxybenzene (21-1) (1.00 g, 3.20 mmol, 1.00 eq) in DMF (25 mL) and H₂O (2.5 mL) were added phenylboronic acid (584 mg, 4.79 mmol, 1.50 eq), KOAc (627 mg, 6.39 mmol, 2.00 eq) and Pd(dppf)Cl₂ (234 mg, 320 μmol, 0.100 eq) at 20° C. under N₂. The mixture was stirred at 80° C. for 12 h under N₂ atmosphere. TLC (SiO₂, Petroleum ether:Ethyl acetate=1:0) indicated 2-bromo-4-iodo-1-methoxybenzene (21-1) was consumed completely and a new spot with a little larger polarity than 2-bromo-4-iodo-1-methoxybenzene (21-1) formed. Then water (30 mL) was added to reaction, the aqueous phase was extracted with EtOAc (30 mL*3), the combined organic layers were dried over Na₂SO₄, filtered and the filtrate was concentrated under reduced pressure. The residue was purified by column chromatography (SiO₂, Petroleum ether:Ethyl acetate=1:0) to give the title compound (21-2) (400 mg, 1.52 mmol, 47.6% yield) as a colorless solid.

Synthesis of 2-(4-methoxy-[1,1′-biphenyl]-3-yl)-4,4,5,5-tetramethyl-1,3,2-dioxaborolane (21-3). To a solution of 3-bromo-4-methoxy-1,1′-biphenyl (21-2) (400 mg, 1.52 mmol, 1.00 eq) in dioxane (10 mL) were added (Bpin)₂ (463 mg, 1.82 mmol, 1.20 eq), Pd(dppf)Cl₂ (55.6 mg, 76.0 μmol, 0.0500 eq) and KOAc (298 mg, 3.04 mmol, 2.00 eq) at 20° C. under N₂. The mixture was stirred at 80° C. for 12 h under N₂ atmosphere. TLC (SiO₂, Petroleum ether:Ethyl acetate=20:1) indicated 3-bromo-4-methoxy-1,1′-biphenyl (21-2) was consumed completely and a new spot with larger polarity than 3-bromo-4-methoxy-1,1′-biphenyl (21-2) formed. Water (8 mL) was added to reaction, the aqueous phase was extracted with EtOAc (8 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 8:1) to give the title compound (21-3) (300 mg, 967 μmol, 63.6% yield) as a yellow oil.

Synthesis of 2,5-dichloro-4-(4-methoxy-[1,1′-biphenyl]-3-yl)pyrimidine (21-4). To a solution of 2-(4-methoxy-[1,1′-biphenyl]-3-yl)-4,4,5,5-tetramethyl-1,3,2-dioxaborolane (21-3) (300 mg, 967 μmol, 1.00 eq) in dioxane (6 mL) and H₂O (0.6 mL) were added 2,4,5-trichloropyrimidine (355 mg, 1.93 mmol, 2.00 eq), Na₂CO₃ (205 mg, 1.93 mmol, 2.00 eq) and Pd(PPh₃)₄ (22.4 mg, 19.3 μmol, 0.0200 eq) at 20° C. under N₂. The mixture was stirred at 90° C. for 12 h under N₂ atmosphere. LCMS showed 2-(4-methoxy-[1,1′-biphenyl]-3-yl)-4,4,5,5-tetramethyl-1,3,2-dioxaborolane (21-3) was consumed completely and the desired mass was detected. Water (8 mL) was added to reaction, the aqueous phase was extracted with EtOAc (6 mL*3), the combined organic layers were dried over Na₂SO₄, filtered and the filtrate was concentrated under reduced pressure. The residue was purified by prep-TLC (SiO₂, Petroleum ether:Ethyl acetate=20:1) to give the title compound (21-4) (100 mg, 302 μmol, 31.2% yield) as a white solid. LCMS (ES⁺): m/z 331.0 [M+H]⁺.

Synthesis of 1-(5-((5-chloro-4-(4-methoxy-[1,1′-biphenyl]-3-yl)pyrimidin-2-yl)amino)pyridin-3-yl)pyrrolidin-2-one (Compound 21). To a solution of 2,5-dichloro-4-(4-methoxy-[1,1′-biphenyl]-3-yl)pyrimidine (21-4) (100 mg, 302 μmol, 0.800 eq) in dioxane (5 mL) were added 1-(5-amino-3-pyridyl)pyrrolidin-2-one (102 mg, 415 μmol, 72.0% purity, 1.10 eq), Pd(OAc)₂ (4.24 mg, 18.9 μmol, 0.0500 eq), Cs₂CO₃ (246 mg, 755 μmol, 2.00 eq) and Xantphos (6.55 mg, 11.3 μmol, 0.0300 eq) at 20° C. under N₂. The mixture was stirred at 100° C. for 12 h under N₂ atmosphere. LCMS showed 2,5-dichloro-4-(4-methoxy-[1,1′-biphenyl]-3-yl)pyrimidine (21-4) was consumed completely and the desired mass was detected. The reaction was filtered and the filtrate was concentrated under reduced pressure. The residue was purified by prep-HPLC (column: Phenomenex Luna C18 75*30 mm*3 um; mobile phase: [water (TFA)-ACN]; B %: 35%-65%, 8 min) to give the title compound (Compound 21) (29.1 mg, 60.9 μmol, 16.1% yield, 98.8% purity) as a white solid. LCMS (ES⁺): m/z 472.0 [M+H]⁺.

### Example 22. Synthesis of 1-(5-((5-chloro-4-(5-chloro-[1,1′-biphenyl]-3-yl)pyrimidin-2-yl)amino)pyridin-3-yl)pyrrolidin-2-one (Compound 22)

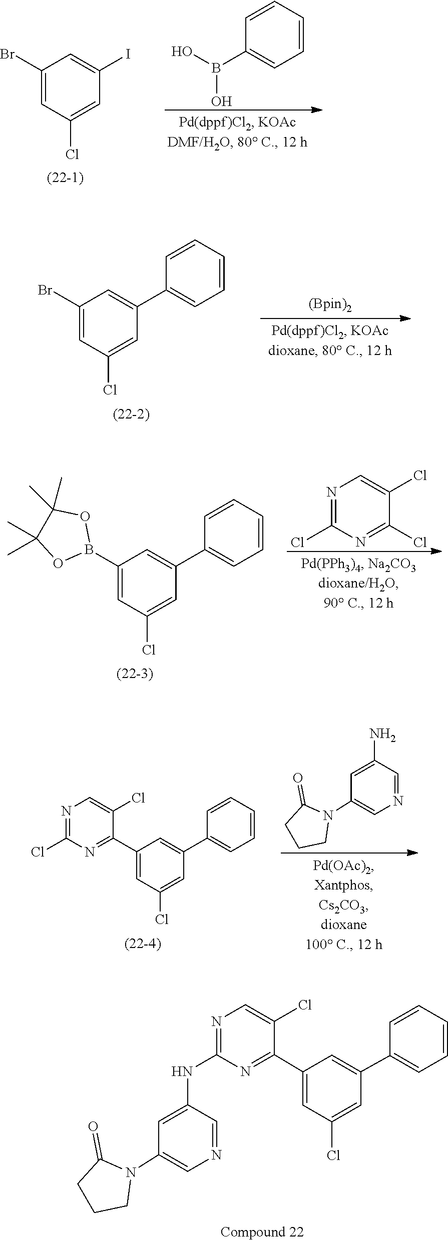

Synthesis of 3-bromo-5-chloro-1,1′-biphenyl (22-2). To a solution of 1-bromo-3-chloro-5-iodobenzene (22-1) (1.00 g, 3.15 mmol, 1.00 eq) in DMF (25 mL) and H₂O (2.5 mL) were added phenylboronic acid (576 mg, 4.73 mmol, 1.50 eq), KOAc (619 mg, 6.30 mmol, 2.00 eq) and Pd(dppf)Cl₂ (231 mg, 315 μmol, 0.100 eq) at 20° C. under N₂. The mixture was stirred at 80° C. for 12 h under N₂ atmosphere. TLC (SiO₂, Petroleum ether:Ethyl acetate=1:0) indicated 1-bromo-3-chloro-5-iodobenzene (22-1) was consumed completely and a new spot with a little larger polarity than 1-bromo-3-chloro-5-iodobenzene (22-1) formed. Then water (20 mL) was added to reaction, the aqueous phase was extracted with EtOAc (15 mL*3), the combined organic layers were dried over Na₂SO₄, filtered and the filtrate was concentrated under reduced pressure. The residue was purified by column chromatography (SiO₂, Petroleum ether:Ethyl acetate=1:0) to give the title compound (22-2) (410 mg, 1.53 mmol, 48.6% yield) as a colorless oil.

Synthesis of 2-(5-chloro-[1,1′-biphenyl]-3-yl)-4,4,5,5-tetramethyl-1,3,2-dioxaborolane (22-3). To a solution of 3-bromo-5-chloro-1,1′-biphenyl (22-2) (410 mg, 1.53 mmol, 1.00 eq) in dioxane (10 mL) were added (Bpin)₂ (467 mg, 1.84 mmol, 1.20 eq), Pd(dppf)Cl₂ (56.1 mg, 76.6 μmol, 0.0500 eq) and KOAc (301 mg, 3.06 mmol, 2.00 eq) at 20° C. under N₂. The mixture was stirred at 80° C. for 12 h under N₂ atmosphere. TLC (SiO₂, Petroleum ether:Ethyl acetate=20:1) indicated 3-bromo-5-chloro-1,1′-biphenyl (22-2) was consumed completely and a new spot with larger polarity than 3-bromo-5-chloro-1,1′-biphenyl (22-2) formed. Water (10 mL) was added to reaction, the aqueous phase was extracted with EtOAc (8 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 8:1) to give the title compound (22-3) (330 mg, 1.05 mmol, 68.5% yield) as a yellow oil.

Synthesis of 2,5-dichloro-4-(5-chloro-[1,1′-biphenyl]-3-yl)pyrimidine (22-4). To a solution of 2-(5-chloro-[1,1′-biphenyl]-3-yl)-4,4,5,5-tetramethyl-1,3,2-dioxaborolane (22-3) (330 mg, 1.05 mmol, 1.00 eq) in dioxane (6 mL) and H₂O (0.6 mL) were added 2,4,5-trichloropyrimidine (385 mg, 2.10 mmol, 2.00 eq), Na₂CO₃ (222 mg, 2.10 mmol, 2.00 eq) and Pd(PPh₃)₄ (24.2 mg, 21.0 μmol, 0.0200 eq) at 20° C. under N₂. The mixture was stirred at 90° C. for 12 h under N₂ atmosphere. LCMS showed 2-(5-chloro-[1,1′-biphenyl]-3-yl)-4,4,5,5-tetramethyl-1,3,2-dioxaborolane (22-3) was consumed completely and the desired mass was detected. Water (5 mL) was added to reaction, the aqueous phase was extracted with EtOAc (8 mL*3), the combined organic layers were dried over Na₂SO₄, filtered and the filtrate was concentrated under reduced pressure. To the residue was added MeOH (5 mL), filtered and the filter cake was dried under reduced pressure to give the title compound (22-4) (150 mg, 447 μmol, 42.6% yield) as a white solid. LCMS (ES⁺): m/z 335.0 [M+H]⁺.

Synthesis of 1-(5-((5-chloro-4-(5-chloro-[1,1′-biphenyl]-3-yl)pyrimidin-2-yl)amino)pyridin-3-yl)pyrrolidin-2-one (Compound 22). To a solution of 2,5-dichloro-4-(5-chloro-[1,1′-biphenyl]-3-yl)pyrimidine (22-4) (150 mg, 447 μmol, 0.800 eq) in dioxane (6 mL) were added 1-(5-amino-3-pyridyl)pyrrolidin-2-one (151 mg, 615 μmol, 72.0% purity, 1.10 eq), Pd(OAc)₂ (6.27 mg, 27.9 μmol, 0.0500 eq), Cs₂CO₃ (364 mg, 1.12 mmol, 2.00 eq) and Xantphos (9.70 mg, 16.8 μmol, 0.0300 eq) at 20° C. under N₂. The mixture was stirred at 100° C. for 12 h under N₂ atmosphere. LCMS showed 2,5-dichloro-4-(5-chloro-[1,1′-biphenyl]-3-yl)pyrimidine (22-4) was consumed completely and the desired mass was detected. Water (5 mL) was added to reaction, the aqueous phase was extracted with EtOAc (5 mL*3), the combined organic layers were dried over Na₂SO₄, filtered and the filtrate was concentrated under reduced pressure. The residue was purified by prep-HPLC (column: Phenomenex luna C18 100*40 mm*3 um; mobile phase: [water (FA)-ACN]; B %: 50%-90%, 8 min) to give the title compound (Compound 22) (31.8 mg, 66.3 μmol, 11.9% yield, 99.4% purity) as a white solid. LCMS (ES⁺): m/z 476.0 [M+H]⁺.

### Example 24. Synthesis of 1-(5-((5-chloro-4-(3-phenylpiperidin-1-yl)pyrimidin-2-yl)amino)pyridin-3-yl)pyrrolidin-2-one (Compound 24)

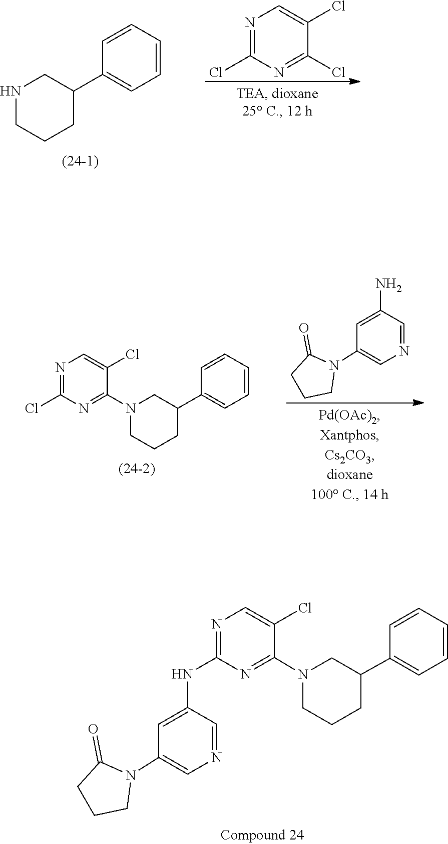

Synthesis of 2,5-dichloro-4-(3-phenylpiperidin-1-yl)pyrimidine (24-2) To a solution of 3-phenylpiperidine (24-1) (316 mg, 1.96 mmol, 1.20 eq) in dioxane (8 mL) were added TEA (182 mg, 1.80 mmol, 250 μL, 1.10 eq) and 2,4,5-trichloropyrimidine (300 mg, 1.64 mmol, 1.00 eq) at 25° C. The mixture was stirred at 25° C. for 12 h. LCMS showed 3-phenylpiperidine (24-1) was consumed, desired target MS was detected. The mixture was concentrated in vacuo. The mixture was purified by prep-TLC (SiO₂, PE:EtOAc=10:1) to give the title compound (24-2) (415 mg, crude) as a yellow oil. LCMS (ES⁺): m/z 308.0 [M+H]⁺.

Synthesis of 1-(5-((5-chloro-4-(3-phenylpiperidin-1-yl)pyrimidin-2-yl)amino)pyridin-3-yl)pyrrolidin-2-one (Compound 24). To a solution of 2,5-dichloro-4-(3-phenylpiperidin-1-yl)pyrimidine (24-2) (300 mg, 973 μmol, 0.800 eq) and 1-(5-amino-3-pyridyl)pyrrolidin-2-one (308 mg, 1.34 mmol, 77.0% purity, 1.10 eq) in dioxane (5 mL) were added Cs₂CO₃ (793 mg, 2.43 mmol, 2.00 eq), Pd(OAc)₂ (13.7 mg, 60.8 μmol, 0.0500 eq) and Xantphos (21.1 mg, 36.5 μmol, 0.0300 eq) at 20° C., then mixture was stirred at 100° C. for 12 h under N₂ atmosphere. LCMS showed 2,5-dichloro-4-(3-phenylpiperidin-1-yl)pyrimidine (24-2) remained, desired target MS was detected. Then Cs₂CO₃ (793 mg, 2.43 mmol, 2.00 eq), Pd(OAc)₂ (13.7 mg, 60.8 μmol, 0.0500 eq) and Xantphos (21.1 mg, 36.5 μmol, 0.0300 eq) were added to the mixture, the mixture was stirred at 100° C. for 2 h under N₂ atmosphere. LCMS showed 2,5-dichloro-4-(3-phenylpiperidin-1-yl)pyrimidine (24-2) was consumed, desired target MS was detected. H₂O (5 mL) was added to the solution, the mixture was extracted with EtOAc (5 mL*3), then the combined organic lays were dried over Na₂SO₄, then concentrated in vacuo to give the crude product. The residue was purified by prep-HPLC (column: Phenomenex Luna C18 75*30 mm*3 um; mobile phase: [water (FA)-ACN]; B %: 20%-60%, 8 min) to give the title compound (Compound 24) (60.0 mg, 129 μmol, 10.6% yield, 96.5% purity) as a white solid. LCMS (ES⁺): m/z 449.1 [M+H]⁺.

### Example 26. Synthesis of 1-(5-((5-chloro-4-(1-methyl-1H-pyrazol-4-yl)pyrimidin-2-yl)amino)pyridin-3-yl)pyrrolidin-2-one (Compound 90)

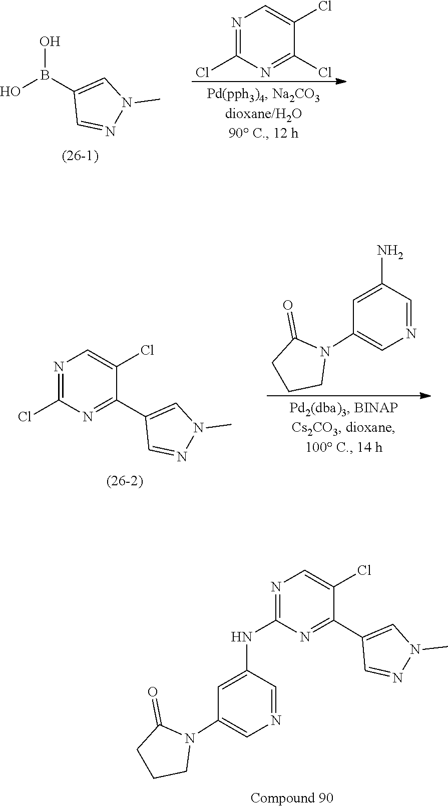

Synthesis of 2,5-dichloro-4-(1-methyl-1H-pyrazol-4-yl)pyrimidine (26-2). To a solution of 1-methyl-1H-pyrazol-4-yl)boronic acid (26-1) (268 mg, 2.13 mmol, 1.30 eq), 2,4,5-trichloropyrimidine (300 mg, 1.64 mmol, 1.00 eq) in dioxane (5 mL) and H₂O (0.5 mL) were added Pd(PPh₃)₄ (37.8 mg, 32.7 μmol, 0.0200 eq) and Na₂CO₃ (347 mg, 3.27 mmol, 2.00 eq) at 25° C., the mixture was stirred at 90° C. for 12 h under N₂ atmosphere. LCMS showed 1-methyl-1H-pyrazol-4-yl)boronic acid (26-1) was consumed, desired target MS was detected. H₂O (5 mL) was added to the solution, the mixture was extracted with EtOAc (5 mL*3), then the combined organic layers were dried over Na₂SO₄, then concentrated in vacuo to give the crude product. The mixture was purified by prep-TLC (SiO₂, PE:EtOAc=1:1) to give the title compound (26-2) (374 mg, crude) as a yellow oil. LCMS (ES⁺): m/z 228.9 [M+H]⁺.

Synthesis of 1-(5-((5-chloro-4-(1-methyl-1H-pyrazol-4-yl)pyrimidin-2-yl)amino)pyridin-3-yl)pyrrolidin-2-one (Compound 90). To a solution of 2,5-dichloro-4-(1-methyl-1H-pyrazol-4-yl)pyrimidine (26-2) (360 mg, 1.57 mmol, 1.00 eq) and 1-(5-aminopyridin-3-yl)pyrrolidin-2-one (398 mg, 1.73 mmol, 77.0% purity, 1.10 eq) in dioxane (5 mL) were added BINAP (58.7 mg, 94.3 μmol, 0.0600 eq), Pd₂(dba)₃ (57.6 mg, 62.9 μmol, 0.0400 eq) and Cs₂CO₃ (1.02 g, 3.14 mmol, 2.00 eq) at 25° C. The mixture was stirred at 100° C. for 12 h under N₂ atmosphere. LCMS showed 2,5-dichloro-4-(1-methyl-1H-pyrazol-4-yl)pyrimidine (26-2) remained, desired target MS was detected, but not well. Then BINAP (58.7 mg, 94.3 μmol, 0.0600 eq), Pd₂(dba)₃ (57.6 mg, 62.9 μmol, 0.0400 eq) and Cs₂CO₃ (1.02 g, 3.14 mmol, 2.00 eq) were added to the mixture, the mixture was stirred at 100° C. for 2 h under N₂ atmosphere. LCMS showed 2,5-dichloro-4-(1-methyl-1H-pyrazol-4-yl)pyrimidine (26-2) was consumed, desired target MS was detected. The mixture was concentrated in vacuo. The residue was purified by prep-HPLC (column: Phenomenex Luna C18 75*30 mm*3 um; mobile phase: [water (FA)-ACN]; B %: 20%-55%, 8 min) to give the title compound ( ) (102 mg, 271 μmol, 17.2% yield, 98.0% purity) as a white solid. LCMS (ES⁺): m/z 370.0 [M+H]⁺.

### Example 27. Synthesis of 1-(5-((5-chloro-4-(3-(piperidin-4-yl)phenyl)pyrimidin-2-yl)amino)pyridin-3-yl)pyrrolidin-2-one (Compound 26)

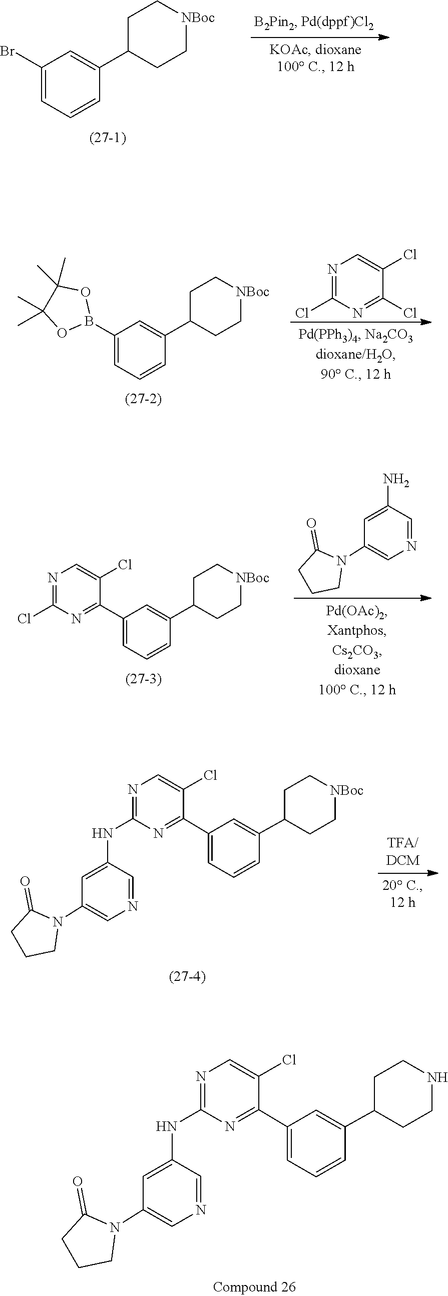

Synthesis of tert-butyl 4-(3-(4,4,5,5-tetramethyl-1,3,2-dioxaborolan-2-yl)phenyl)piperidine-1-carboxylate (27-2). To a solution of tert-butyl 4-(3-bromophenyl)piperidine-1-carboxylate (27-1) (3.00 g, 8.82 mmol, 1.00 eq) and B₂Pin₂ (2.68 g, 10.6 mmol, 1.20 eq) in dioxane (30 mL) were added Pd(dppf)Cl₂ (323 mg, 441 μmol, 0.0500 eq) and KOAc (1.73 g, 17.6 mmol, 2.00 eq) at 20° C. under N₂ atmosphere. The resulting mixture was stirred at 100° C. for 12 h under N₂. LCMS showed tert-butyl 4-(3-bromophenyl)piperidine-1-carboxylate (27-1) remained and the desired mass was detected. The mixture was diluted with H₂O (30 mL) and then extracted with EtOAc (30 mL*3), the combined organic layers were dried over Na₂SO₄, filtered and the filtrate was concentrated under reduced pressure. The mixture was purified by MPLC (SiO₂, Petroleum ether:Ethyl acetate=1:0 to 1:1) to give the title compound (27-2) (1.50 g, 3.87 mmol, 43.9% yield) as a yellow oil. LCMS (ES⁺): m/z 332.1 [M+H−56]⁺.

Synthesis of tert-butyl 4-(3-(2,5-dichloropyrimidin-4-yl)phenyl)piperidine-1-carboxylate (27-3). To a solution of tert-butyl 4-(3-(4,4,5,5-tetramethyl-1,3,2-dioxaborolan-2-yl)phenyl)piperidine-1-carboxylate (27-2) (1.50 g, 3.87 mmol, 1.00 eq) and 2,4,5-trichloropyrimidine (1.42 g, 7.74 mmol, 2.00 eq) in dioxane (50 mL) and H₂O (10 mL) were added Pd(PPh₃)₄ (89.5 mg, 77.5 μmol, 0.0200 eq) and Na₂CO₃ (821 mg, 7.74 mmol, 2.00 eq) at 20° C. under N₂ atmosphere. The resulting mixture was stirred at 90° C. for 12 h under N₂. LCMS showed tert-butyl 4-(3-(4,4,5,5-tetramethyl-1,3,2-dioxaborolan-2-yl)phenyl)piperidine-1-carboxylate (27-2) remained and the desired mass was detected. The mixture was diluted with H₂O (15 mL) and then extracted with EtOAc (15 mL*3), the combined organic layers were dried over Na₂SO₄, filtered and the filtrate was concentrated under reduced pressure. The mixture was purified by MPLC (SiO₂, Petroleum ether:Ethyl acetate=1:0 to 1:1) to give the title compound (27-3) (1.40 g, 3.43 mmol, 88.5% yield) as a yellow oil. LCMS (ES⁺): m/z 352.0 [M+H−56]⁺.

Synthesis of tert-butyl 4-(3-(5-chloro-2-((5-(2-oxopyrrolidin-1-yl)pyridin-3-yl)amino)pyrimidin-4-yl)phenyl)piperidine-1-carboxylate (27-4). To a solution of tert-butyl 4-(3-(2,5-dichloropyrimidin-4-yl)phenyl)piperidine-1-carboxylate (27-3) (1.40 g, 3.43 mmol, 1.00 eq) and 1-(5-amino-3-pyridyl)pyrrolidin-2-one (868 mg, 3.43 mmol, 70.0% purity, 1.00 eq) in dioxane (50 mL) were added Xantphos (198 mg, 343 μmol, 0.100 eq), Pd(OAc)₂ (77.0 mg, 343 μmol, 0.100 eq) and Cs₂CO₃ (1.12 g, 3.43 mmol, 1.00 eq) at 20° C. under N₂ atmosphere. The mixture was stirred at 100° C. for 12 h under N₂. LCMS showed tert-butyl 4-(3-(2,5-dichloropyrimidin-4-yl)phenyl)piperidine-1-carboxylate (27-3) was consumed and the desired mass was detected. The mixture was diluted with H₂O (10 mL) and then extracted with EtOAc (20 mL*3), the combined organic layers were dried over Na₂SO₄, filtered and the filtrate was concentrated under reduced pressure. The mixture was purified by MPLC (SiO₂, Petroleum ether:Ethyl acetate=1:0 to 0:1) to give the title compound (27-4) (1.80 g, 3.28 mmol, 95.6% yield) as a yellow oil. LCMS (ES⁺): m/z 549.2 [M+H]⁺.

Synthesis of 1-(5-((5-chloro-4-(3-(piperidin-4-yl)phenyl)pyrimidin-2-yl)amino)pyridin-3-yl)pyrrolidin-2-one (Compound 26). To a solution of tert-butyl 4-(3-(5-chloro-2-((5-(2-oxopyrrolidin-1-yl)pyridin-3-yl)amino)pyrimidin-4-yl)phenyl)piperidine-1-carboxylate (27-4) (1.00 g, 1.82 mmol, 1.00 eq) in DCM (10 mL) was added TFA (2 mL) at 20° C. The mixture was stirred at 20° C. for 12 h. LCMS showed tert-butyl 4-(3-(5-chloro-2-((5-(2-oxopyrrolidin-1-yl)pyridin-3-yl)amino)pyrimidin-4-yl)phenyl)piperidine-1-carboxylate (27-4) was consumed and the desired mass was detected. The mixture was concentrated under reduced pressure. 200 mg of it was purified by prep-HPLC (column: Phenomenex Luna C18 75*30 mm*3 um; mobile phase: [water (FA)-ACN]; B %: 1%-20%, 8 min) to give the title compound (Compound 26) (71.7 mg, 126 μmol, 6.91% yield, 98.9% purity) as a white solid. LCMS (ES⁺): m/z 449.1 [M+H]⁺.

### Example 28. Synthesis of 1-(5-((5-chloro-4-(3-(1-methylpiperidin-4-yl)phenyl)pyrimidin-2-yl)amino)pyridin-3-yl)pyrrolidin-2-one (Compound 27)

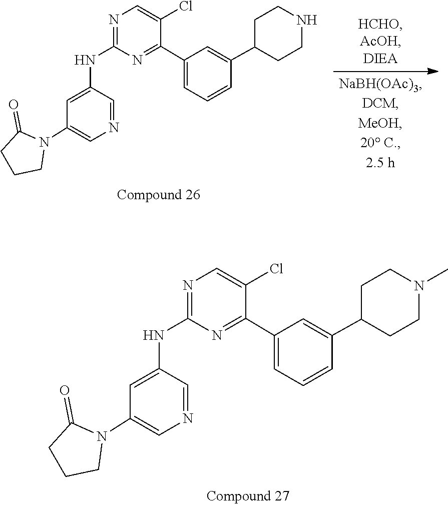 [SMILES: O=C1CCCN1c1cncc(Nc2ncc(Cl)c(-c3cccc(C4CCNCC4)c3)n2)c1 ; CN1CCC(c2cccc(-c3nc(Nc4cncc(N5CCCC5=O)c4)ncc3Cl)c2)CC1]

Synthesis of 1-(5-((5-chloro-4-(3-(I-methylpiperidin-4-yl)phenyl)pyrimidin-2-yl)amino)pyridin-3-yl)pyrrolidin-2-one (Compound 27). To a solution of 1-(5-((5-chloro-4-(3-(piperidin-4-yl)phenyl)pyrimidin-2-yl)amino)pyridin-3-yl)pyrrolidin-2-one (Compound 26) (300 mg, 668 μmol, 1.00 eq) in DCM (3 mL) and MeOH (1.5 mL) was added DIEA (86.4 mg, 668 μmol, 116 μL, 1.00 eq) to adjust pH to 8, then AcOH (4.01 mg, 66.8 μmol, 3.82 μL, 0.100 eq) and HCHO (922 mg, 11.4 mmol, 846 μL, 37.0% purity, 17.0 eq) were added to the mixture, the mixture was stirred at 20° C. for 0.5 h, then NaBH(OAc)₃ (170 mg, 802 μmol, 1.20 eq) was added to the mixture, the mixture was stirred at 20° C. for 2 h. LCMS showed 1-(5-((5-chloro-4-(3-(piperidin-4-yl)phenyl)pyrimidin-2-yl)amino)pyridin-3-yl)pyrrolidin-2-one (Compound 26) was consumed and the desired mass was detected. The mixture was concentrated under reduced pressure to give a residue. The residue was purified by prep-HPLC (column: Phenomenex Luna C18 75*30 mm*3 um; mobile phase: [water (FA)-ACN]; B %: 15%-50%, 8 min) to give the title compound (Compound 27) (203 mg, 433 μmol, 64.8% yield, 98.9% purity) as a white solid. LCMS (ES⁺): m/z 463.0 [M+H]⁺.

### Example 29. Synthesis of 1-(5-((4-(3-(1-acetylpiperidin-4-yl)phenyl)-5-chloropyrimidin-2-yl)amino)pyridin-3-yl)pyrrolidin-2-one (Compound 28)

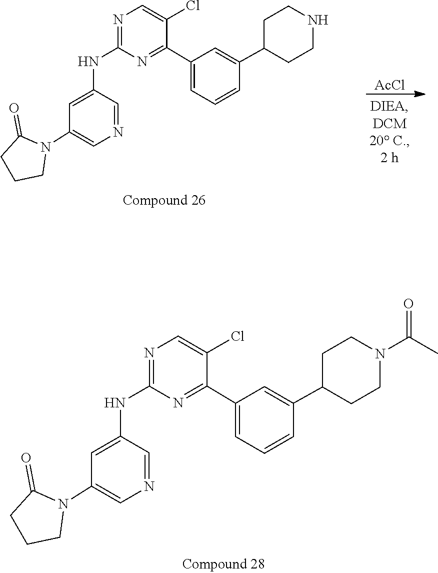 [SMILES: "AcCl" ; O=C1CCCN1c1cncc(Nc2ncc(Cl)c(-c3cccc(C4CCNCC4)c3)n2)c1 ; CC(=O)N1CCC(c2cccc(-c3nc(Nc4cncc(N5CCCC5=O)c4)ncc3Cl)c2)CC1]

Synthesis of 1-(5-((4-(3-(1-acetylpiperidin-4-yl)phenyl)-5-chloropyrimidin-2-yl)amino)pyridin-3-yl)pyrrolidin-2-one (Compound 28). To a solution of 1-(5-((5-chloro-4-(3-(piperidin-4-yl)phenyl)pyrimidin-2-yl)amino)pyridin-3-yl)pyrrolidin-2-one (Compound 26) (300 mg, 533 μmol, 1.00 eq, TFA) and DIEA (207 mg, 1.60 mmol, 278 μL, 3.00 eq) in DCM (3 mL) was added AcCl (37.7 mg, 480 μmol, 34.2 μL, 0.900 eq) at 0° C. The mixture was stirred at 20° C. for 2 h. LCMS showed that 1-(5-((5-chloro-4-(3-(piperidin-4-yl)phenyl)pyrimidin-2-yl)amino)pyridin-3-yl)pyrrolidin-2-one (Compound 26) was consumed and the desired mass was detected. The mixture was concentrated under reduced pressure. This mixture was purified by acidic prep-HPLC (column: Phenomenex Luna C18 75*30 mm*3 um; mobile phase: [water (FA)-ACN]; B %: 15%-35%, 8 min) to give the title compound (Compound 28) (80.1 mg, 163 μmol, 30.6% yield, 99.9% purity) as white solid. LCMS (ES⁺): m/z 491.0 [M+H]⁺.

### Example 30. Synthesis of (S)-1-(5-chloro-2-((5-(2-oxopyrrolidin-1-yl)pyridin-3-yl)amino)pyrimidin-4-yl)-5-phenylpyrrolidin-2-one and (R)-1-(5-chloro-2-((5-(2-oxopyrrolidin-1-yl)pyridin-3-yl)amino)pyrimidin-4-yl)-5-phenylpyrrolidin-2-one (Compound 29, Enantiomer A and Enantiomer B)

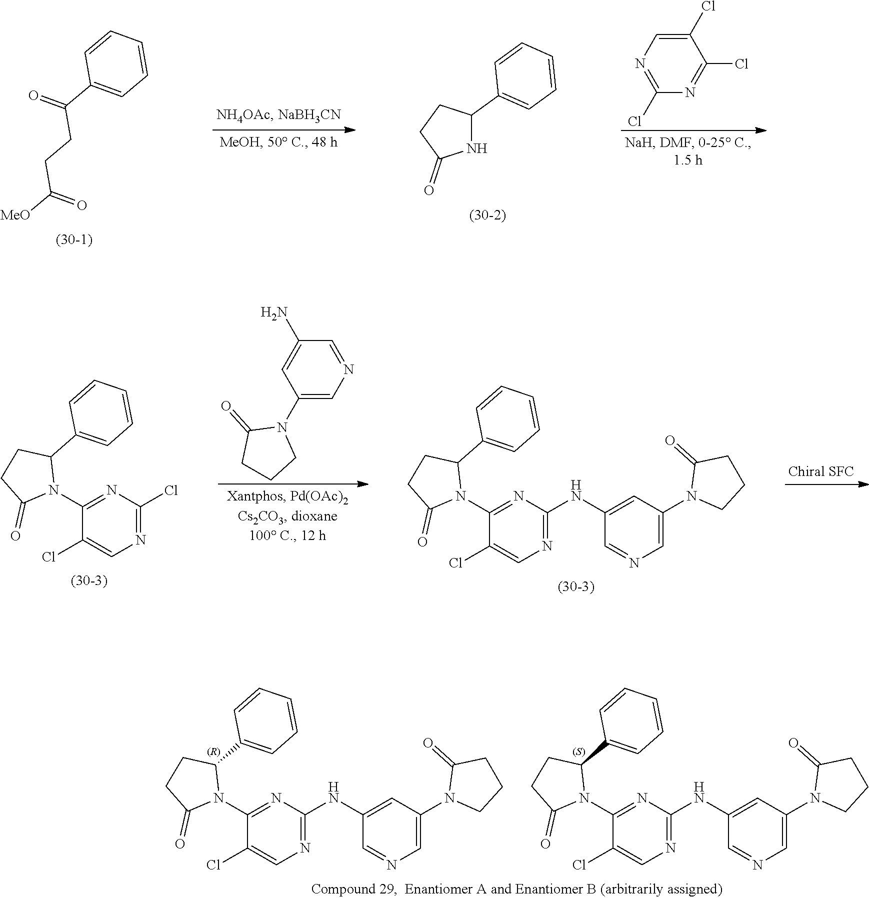

Synthesis of 5-phenylpyrrolidin-2-one (30-2). To a solution of methyl 4-oxo-4-phenylbutanoate 30-1 (10.0 g, 52.0 mmol, 1.00 eq) and acetic acid; ammonia (116 g, 1.51 μmol, 29.0 eq) in MeOH (400 mL) was added NaBH₃CN (16.4 g, 260 mmol, 5.00 eq). The mixture was stirred at 50° C. for 48 h. LCMS showed methyl 4-oxo-4-phenylbutanoate 30-1 was consumed completely and desired mass was detected. The reaction mixture was concentrated. The reaction mixture was partitioned between H₂O (100 mL) and EtOAc (100 mL). The organic phase was separated, dried over Na₂SO₄, filtered and concentrated under reduced pressure to give the title compound (30-2) (8.30 g, 51.5 mmol, 99.0% yield) as a brown solid. LCMS (ES⁺): m/z 162.1 [M+H]⁺.

Synthesis of 1-(2,5-dichloropyrimidin-4-yl)-5-phenylpyrrolidin-2-one (30-3). To a solution of 5-phenylpyrrolidin-2-one (30-2) (2.00 g, 12.4 mmol, 1.00 eq) in DMF (20 mL) was added NaH (992 mg, 24.8 mmol, 60.0% purity, 2.00 eq) at 0° C. The reaction mixture was stirred at 0° C. for 30 min. Then 2,4,5-trichloropyrimidine (2.05 g, 11.2 mmol, 0.900 eq) was added at 0° C. The mixture was stirred at 25° C. for 1 h. LCMS showed 5-phenylpyrrolidin-2-one (30-2) was consumed completely and desired mass was detected. The reaction was quenched by addition of saturated aqueous NH₄Cl (50 mL), extracted with EtOAc (60 mL*3). The combined organic layers were dried over Na₂SO₄, filtered and concentrated under reduced pressure. The residue was purified by flash silica gel chromatography (ISCO®; 40 g SepaFlash® Silica Flash Column, Eluent of 0-20% Ethyl acetate/Petroleum ether gradient @100 mL/min) to give the title compound (30-3) (500 mg, 1.22 mmol, 9.81% yield, 75.0% purity) as a white solid. LCMS (ES⁺): m/z 308.1 [M+H]⁺.

Synthesis of (S)-1-(5-chloro-2-((5-(2-oxopyrrolidin-1-yl)pyridin-3-yl)amino)pyrimidin-4-yl)-5-phenylpyrrolidin-2-one (Compound 29) and (R)-1-(5-chloro-2-((5-(2-oxopyrrolidin-1-yl)pyridin-3-yl)amino)pyrimidin-4-yl)-5-phenylpyrrolidin-2-one (Compound 31). To a solution of 1-(2,5-dichloropyrimidin-4-yl)-5-phenylpyrrolidin-2-one (30-3) (400 mg, 1.30 mmol, 1.00 eq), 1-(5-amino-3-pyridyl)pyrrolidin-2-one (253 mg, 1.43 mmol, 1.10 eq), Xantphos (150 mg, 260 μmol, 0.200 eq) and Cs₂CO₃ (845 mg, 2.60 mmol, 2.00 eq) in dioxane (5 mL) was added Pd(OAc)₂ (58.3 mg, 260 μmol, 0.200 eq). The mixture was stirred at 100° C. for 12 h under N₂. LCMS showed 1-(2,5-dichloropyrimidin-4-yl)-5-phenylpyrrolidin-2-one (30-3) was consumed completely and desired mass was detected. The reaction mixture was filtered and the filtrate was concentrated under reduced pressure to remove solvent. The residue was purified by prep-HPLC (column: Phenomenex luna C18 80*40 mm*3 um; mobile phase: [water (FA)-ACN]; B %: 10%-50%, 8 min). The racemate was separated by Chiral SFC (column: (s,s) WHELK-O1 (250 mm*30 mm, Sum); mobile phase: [0.1% NH₃H₂O MeOH]; B %: 50%-50%, 16 min) to give the title compound (Enantiomer A) (17.0 mg, 37.9 μmol, 2.92% yield, 100% purity) as a white solid and the title compound (Enantiomer B) (23.3 mg, 51.9 μmol, 4.00% yield, 100% purity) as a white solid. The two isomers were assigned arbitrarily. LCMS (ES⁺): m/z 449.1 [M+H]⁺.

### Example 31. Synthesis of 1-(5-((5-chloro-4-(3-(piperidin-3-yl)phenyl)pyrimidin-2-yl)amino)pyridin-3-yl)pyrrolidin-2-one (Compound 30)

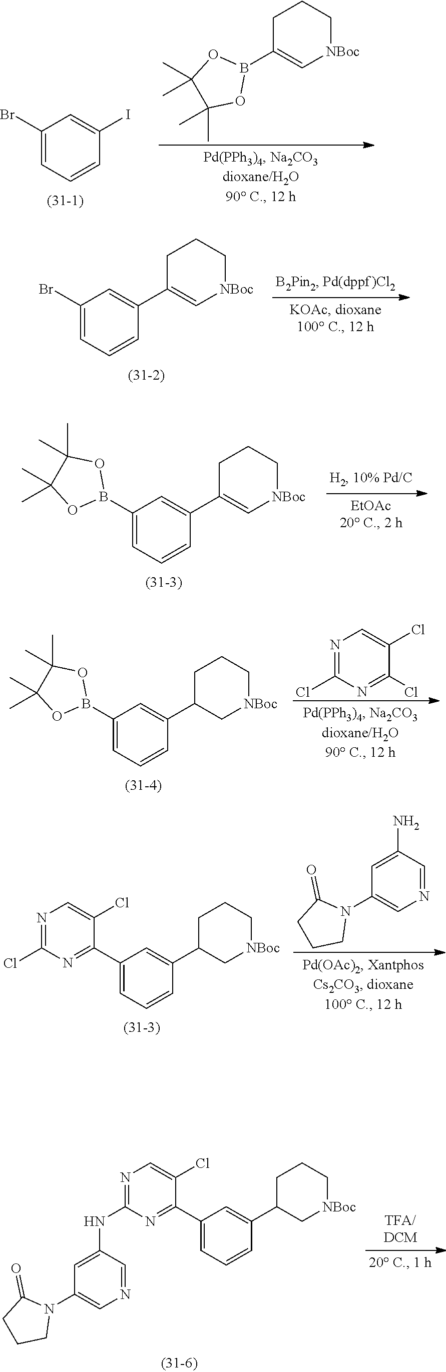

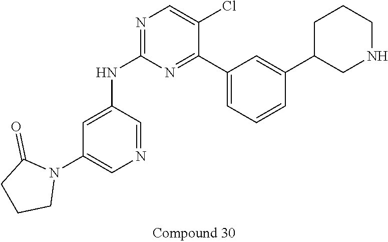 [SMILES: O=C1CCCN1c1cncc(Nc2ncc(Cl)c(-c3cccc(C4CCCNC4)c3)n2)c1]

Synthesis of tert-butyl 5-(3-bromophenyl)-3,4-dihydropyridine-1(2H)-carboxylate (31-2). To a solution of 1-bromo-3-iodobenzene (31-1) (549 mg, 1.94 mmol, 247 μL, 1.20 eq) and tert-butyl 5-(4,4,5,5-tetramethyl-1,3,2-dioxaborolan-2-yl)-3,4-dihydro-2H-pyridine-1-carboxylate (500 mg, 1.62 mmol, 1.00 eq) in dioxane (10 mL) and H₂O (2 mL) were added Na₂CO₃ (343 mg, 3.23 mmol, 2.00 eq) and Pd(PPh₃)₄ (37.4 mg, 32.3 μmol, 0.0200 eq) at 20° C. under N₂ atmosphere. The mixture was stirred at 90° C. for 12 h under N₂. LCMS showed that 31-1 was consumed and the desired mass was detected. To the mixture was added H₂O (20 mL) and the mixture was extracted with EtOAc (20 mL*3). The combined organic layers were dried over Na₂SO₄, filtered and the filtrate was concentrated under reduced pressure. The mixture was purified by prep-TLC (SiO₂, PE:EtOAc=10:1) to give the title compound (31-2) (350 mg, 1.03 mmol, 64.0% yield) as a colorless oil. LCMS (ES⁺): m/z 281.9 [M+H−56]⁺.

Synthesis of tert-butyl 5-(3-(4,4,5,5-tetramethyl-1,3,2-dioxaborolan-2-yl)phenyl)-3,4-dihydropyridine-1(2H)-carboxylate (31-3). To a solution of tert-butyl 5-(3-bromophenyl)-3,4-dihydropyridine-1(2H)-carboxylate (31-2) (350 mg, 1.03 μmol, 1.00 eq) and B₂Pin₂ (315 mg, 1.24 mmol, 1.20 eq) in dioxane (6 mL) were added KOAc (203 mg, 2.07 mmol, 2.00 eq), Pd(dppf)Cl₂ (7.57 mg, 10.4 μmol, 0.0100 eq) at 20° C. under N₂ atmosphere. The mixture was stirred at 100° C. for 12 h under N₂. LCMS showed that tert-butyl 5-(3-bromophenyl)-3,4-dihydropyridine-1(2H)-carboxylate (31-2) was consumed and the desired mass was detected. To the mixture was added H₂O (10 mL) and the mixture was extracted with EtOAc (10 mL*3). The combined organic layers were dried over Na₂SO₄, filtered and the filtrate was concentrated under reduced pressure. The mixture was purified by prep-TLC (SiO₂, PE:EtOAc=10:1) to give the title compound (31-3) (350 mg, 908 μmol, 87.8% yield) as a white solid. LCMS (ES⁺): m/z 286.1 [M+H−100]⁺.

Synthesis of tert-butyl 3-(3-(4,4,5,5-tetramethyl-1,3,2-dioxaborolan-2-yl)phenyl)piperidine-1-carboxylate (31-4). To a solution of tert-butyl 5-(3-(4,4,5,5-tetramethyl-1,3,2-dioxaborolan-2-yl)phenyl)-3,4-dihydropyridine-1(2H)-carboxylate (31-3) (150 mg, 389 μmol, 1.00 eq) in EtOAc (5 mL) was added 10% Pd/C (150 mg, 50.0% purity) at 20° C. The resulting mixture was stirred at 20° C. under H₂ (15 psi) for 2 h. LCMS showed tert-butyl 5-(3-(4,4,5,5-tetramethyl-1,3,2-dioxaborolan-2-yl)phenyl)-3,4-dihydropyridine-1(2H)-carboxylate (31-3) was consumed and the desired mass was detected. The mixture was filtered and the filtrate was concentrated under reduced pressure. The mixture was purified by prep-TLC (SiO₂, PE:EtOAc=1:2) to give the title compound (31-4) (170 mg, 395 μmol, 27.1% yield) as a yellow oil. LCMS (ES⁺): m/z 288.1 [M+H−100]⁺.

Synthesis of tert-butyl 3-(3-(2,5-dichloropyrimidin-4-yl)phenyl)piperidine-1-carboxylate (31-5). To a solution of tert-butyl 3-(3-(4,4,5,5-tetramethyl-1,3,2-dioxaborolan-2-yl)phenyl)piperidine-1-carboxylate (31-4) (290 mg, 749 μmol, 1.00 eq) and 2,4,5-trichloropyrimidine (275 mg, 1.50 mmol, 2.00 eq) in dioxane (5 mL) and H₂O (1 mL) were added Na₂CO₃ (159 mg, 1.50 mmol, 2.00 eq) and Pd(PPh₃)₄ (17.3 mg, 15.0 μmol, 0.0200 eq) at 20° C. under N₂ atmosphere. The mixture was stirred at 90° C. for 12 h under N₂. LCMS showed that tert-butyl 3-(3-(4,4,5,5-tetramethyl-1,3,2-dioxaborolan-2-yl)phenyl)piperidine-1-carboxylate (31-4) was consumed and the desired mass was detected. To the mixture was added H₂O (10 mL) and the mixture was extracted with EtOAc (10 mL*3), the combined organic layers were dried over Na₂SO₄, filtered and the filtrate was concentrated under reduced pressure. The mixture was purified by prep-TLC (SiO₂, PE:EtOAc=5:1) to give the title compound (31-5) (180 mg, 441 μmol, 58.9% yield) as a yellow oil. LCMS (ES⁺): m/z 352.0 [M+H−56]⁺.

Synthesis of tert-butyl 3-(3-(5-chloro-2-((5-(2-oxopyrrolidin-1-yl)pyridin-3-yl)amino)pyrimidin-4-yl)phenyl)piperidine-1-carboxylate (31-6). To a solution of tert-butyl 3-(3-(2,5-dichloropyrimidin-4-yl)phenyl)piperidine-1-carboxylate (31-5) (180 mg, 441 μmol, 0.800 eq) and 1-(5-amino-3-pyridyl)pyrrolidin-2-one (153 mg, 606 μmol, 70.0% purity, 1.10 eq) in dioxane (3 mL) were added Cs₂CO₃ (359 mg, 1.10 mmol, 2.00 eq), Xantphos (31.9 mg, 55.1 μmol, 0.100 eq) and Pd(OAc)₂ (12.4 mg, 55.1 μmol, 0.100 eq) at 20° C. under N₂ atmosphere, then the solution was stirred at 100° C. for 12 h under N₂. LCMS showed that tert-butyl 3-(3-(2,5-dichloropyrimidin-4-yl)phenyl)piperidine-1-carboxylate (31-5) was consumed and the desired mass was detected. To the mixture was added H₂O (10 mL) and the mixture was extracted with EtOAc (10 mL*3), the combined organic layers were dried over Na₂SO₄, filtered and the filtrate was concentrated under reduced pressure. The mixture was purified by prep-TLC (SiO₂, PE:EtOAc=0:1) to give the title compound (31-6) (130 mg, 237 μmol, 43.0% yield) as a white solid. LCMS (ES⁺): m/z 449.1 [M+H−100]⁺.

Synthesis of 1-(5-((5-chloro-4-(3-(piperidin-3-yl)phenyl)pyrimidin-2-yl)amino)pyridin-3-yl)pyrrolidin-2-one (Compound 30). A solution of tert-butyl 3-(3-(5-chloro-2-((5-(2-oxopyrrolidin-1-yl)pyridin-3-yl)amino)pyrimidin-4-yl)phenyl)piperidine-1-carboxylate (31-6) (130 mg, 237 μmol, 1.00 eq) in TFA (1 mL) and DCM (5 mL) was stirred at 20° C. for 1 h. LCMS showed that tert-butyl 3-(3-(5-chloro-2-((5-(2-oxopyrrolidin-1-yl)pyridin-3-yl)amino)pyrimidin-4-yl)phenyl)piperidine-1-carboxylate (31-6) was consumed and the desired mass was detected. The mixture was concentrated under reduced pressure. The mixture was purified by prep-HPLC (Phenomenex Luna C18 200*40 mm*10 um; mobile phase: [water (FA)-ACN]; B %: 15%-40%, 8 min) to give the title compound (Compound 30) (31.8 mg, 69.9 μmol, 29.5% yield, 98.7% purity) as a yellow solid. LCMS (ES⁺): m/z 449.2 [M+H]⁺.

### Example 32. Synthesis of 1-(5-((4-([1,1′-biphenyl]-3-yl)-5-chloropyrimidin-2-yl)amino)pyridin-3-yl)-3-methylimidazolidin-2-one (Compound 32)

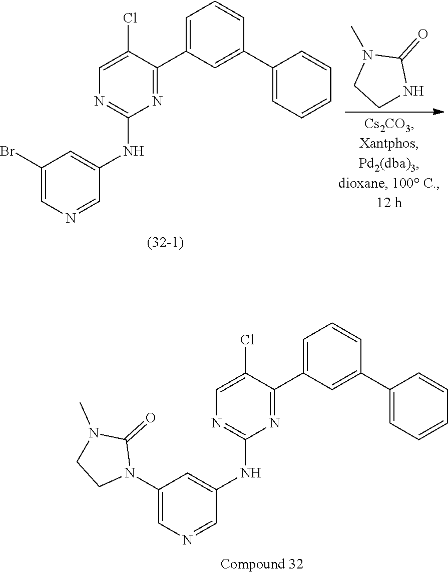

Synthesis of 1-(5-((4-([1,1′-biphenyl]-3-yl)-5-chloropyrimidin-2-yl)amino)pyridin-3-yl)-3-methylimidazolidin-2-one (Compound 32). To a solution of 4-([1,1′-biphenyl]-3-yl)-N-(5-bromopyridin-3-yl)-5-chloropyrimidin-2-amine (32-1) (prepared as described in Example 1) (100 mg, 228 μmol, 1.00 eq), 1-methylimidazolidin-2-one (25.2 mg, 251 μmol, 1.10 eq), Cs₂CO₃ (186 mg, 571 μmol, 2.50 eq) and Pd₂(dba)₃ (20.9 mg, 22.9 μmol, 0.100 eq) in dioxane (2 mL) was added Xantphos (26.4 mg, 45.7 μmol, 0.200 eq). The mixture was stirred at 100° C. for 12 h under N₂. LCMS showed 4-([1,1′-biphenyl]-3-yl)-N-(5-bromopyridin-3-yl)-5-chloropyrimidin-2-amine (32-1) was consumed completely and desired mass was detected. The reaction mixture was concentrated. The residue was purified by prep-HPLC (column: Phenomenex luna C18 80*40 mm*3 um; mobile phase: [water (FA)-ACN]; B %: 25%-65%, 8 min) to give the title compound (Compound 32) (27.0 mg, 57.7 μmol, 25.3% yield, 97.7% purity) as a white solid. LCMS (ES⁺): m/z 457.1 [M+H]⁺.

### Example 33. Synthesis of 1-(5-((5-chloro-4-(3-(1-methylpiperidin-3-yl)phenyl)pyrimidin-2-yl)amino)pyridin-3-yl)pyrrolidin-2-one (Compound 33)

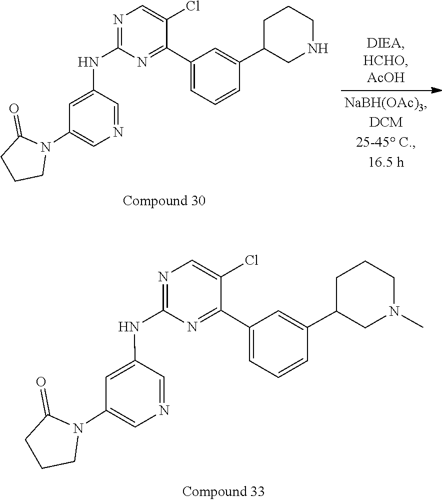 [SMILES: O=C1CCCN1c1cncc(Nc2ncc(Cl)c(-c3cccc(C4CCCNC4)c3)n2)c1 ; CN1CCCC(c2cccc(-c3nc(Nc4cncc(N5CCCC5=O)c4)ncc3Cl)c2)C1]

Synthesis of 1-(5-((5-chloro-4-(3-(I-methylpiperidin-3-yl)phenyl)pyrimidin-2-yl)amino)pyridin-3-yl)pyrrolidin-2-one (Compound 33). To a solution of 1-(5-((5-chloro-4-(3-(piperidin-3-yl)phenyl)pyrimidin-2-yl)amino)pyridin-3-yl)pyrrolidin-2-one (Compound 30) (crude, 180 mg, 320 μmol, 1.00 eq, TFA) in DCM (3 mL) and MeOH (1.5 mL) were added DIEA (41.3 mg, 320 μmol, 55.69 μL, 1.00 eq) to adjust pH to 8, then aq. HCHO (441 mg, 5.44 mmol, 405 μL, 37.0% purity, 17.0 eq) and AcOH (1.92 mg, 32.0 μmol, 1.83 μL, 0.100 eq) were added to the mixture at 25° C., the mixture was stirred at 25° C. for 0.5 h, then NaBH(OAc)₃ (81.3 mg, 384 μmol, 1.20 eq) was added to the mixture at 25° C., the mixture was stirred at 25° C. for 2 h. LCMS showed 1-(5-((5-chloro-4-(3-(piperidin-3-yl)phenyl)pyrimidin-2-yl)amino)pyridin-3-yl)pyrrolidin-2-one (Compound 30) remained, the desired mass was detected. Then aq. HCHO (441 mg, 5.44 mmol, 405 μL, 37.0% purity, 17.0 eq) was added to the mixture, the mixture was stirred at 25° C. for 2 h. Then NaBH(OAc)₃ (339 mg, 1.60 mmol, 5.00 eq) was added to the mixture, the mixture was stirred at 45° C. for 12 h. LCMS showed 1-(5-((5-chloro-4-(3-(piperidin-3-yl)phenyl)pyrimidin-2-yl)amino)pyridin-3-yl)pyrrolidin-2-one (Compound 30) was consumed, the desired mass was detected. The mixture was concentrated under reduced pressure to give a residue. The residue was purified by prep-HPLC (column: Phenomenex Luna C18 75*30 mm*3 um; mobile phase: [water (FA)-ACN]; B %: 1%-50%, 8 min) to give the title compound (Compound 33) (76.5 mg, 164 μmol, 51.2% yield, 99.2% purity) as a white solid. LCMS (ES⁺): m/z 463.1 [M+H]⁺.

### Example 34. Synthesis of 1-(5-((4-(3-(1-acetylpiperidin-3-yl)phenyl)-5-chloropyrimidin-2-yl)amino)pyridin-3-yl)pyrrolidin-2-one (Compound 34)

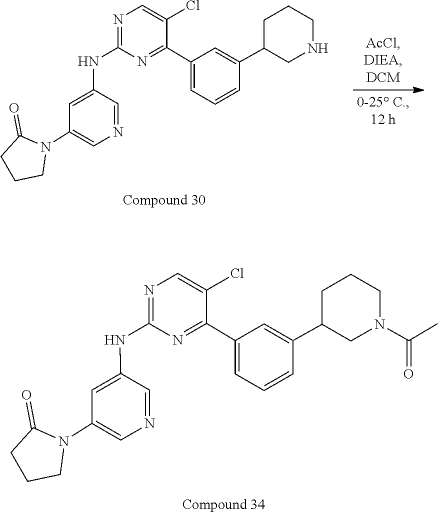 [SMILES: O=C1CCCN1c1cncc(Nc2ncc(Cl)c(-c3cccc(C4CCCNC4)c3)n2)c1 ; CC(=O)N1CCCC(c2cccc(-c3nc(Nc4cncc(N5CCCC5=O)c4)ncc3Cl)c2)C1]

Synthesis of 1-(5-((4-(3-(1-acetylpiperidin-3-yl)phenyl)-5-chloropyrimidin-2-yl)amino)pyridin-3-yl)pyrrolidin-2-one (Compound 34). To a solution of 1-(5-((5-chloro-4-(3-(piperidin-3-yl)phenyl)pyrimidin-2-yl)amino)pyridin-3-yl)pyrrolidin-2-one (Compound 30) (150 mg, 266 μmol, 1.00 eq, TFA) and DIEA (103 mg, 799 μmol, 139 μL, 3.00 eq) in DCM (2 mL) was added acetyl chloride (18.8 mg, 239 μmol, 17.1 μL, 0.900 eq) at 0° C. The mixture was stirred at 25° C. for 15 min. LCMS showed that 1-(5-((5-chloro-4-(3-(piperidin-3-yl)phenyl)pyrimidin-2-yl)amino)pyridin-3-yl)pyrrolidin-2-one (Compound 30) was consumed and the desired mass was detected. To the mixture was added H₂O (5 mL) and the mixture was extracted with EtOAc (5 mL*3). The combined organic layers were dried over Na₂SO₄, filtered and the filtrate was concentrated under reduced pressure. The residue was purified by acidic prep-HPLC (column: Phenomenex luna C18 80*40 mm*3 um; mobile phase: [water (FA)-ACN]; B %: 20%-60%, 8 min) to give the title compound (Compound 34) (37.9 mg, 77.1 μmol, 28.9% yield, 99.9% purity) as white solid. LCMS (ES⁺): m/z 491.1 [M+H]⁺.

### Example 35. Synthesis of 6-(5-((4-([1,1′-biphenyl]-3-yl)-5-chloropyrimidin-2-yl)amino)pyridin-3-yl)-2-methyl-2,6-diazaspiro[3.4]octan-7-one (Compound 35)

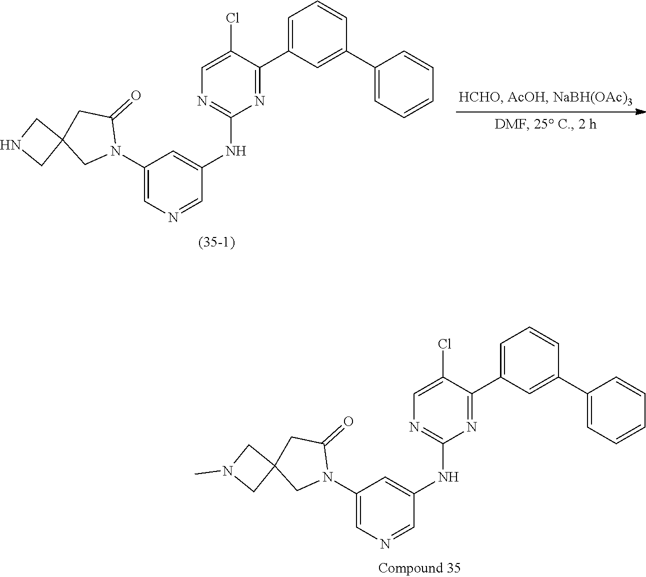 [SMILES: O=C1CC2(CNC2)CN1c1cncc(Nc2ncc(Cl)c(-c3cccc(-c4ccccc4)c3)n2)c1 ; CN1CC2(CC(=O)N(c3cncc(Nc4ncc(Cl)c(-c5cccc(-c6ccccc6)c5)n4)c3)C2)C1]

Synthesis of 6-(5-((4-([1,1′-biphenyl]-3-yl)-5-chloropyrimidin-2-yl)amino)pyridin-3-yl)-2-methyl-2,6-diazaspiro[3.4]octan-7-one (Compound 35). To a stirred solution of 6-(5-((4-([1,1′-biphenyl]-3-yl)-5-chloropyrimidin-2-yl)amino)pyridin-3-yl)-2,6-diazaspiro[3.4]octan-7-one (35-1) (prepared as described in Example 36) (200 mg, 414 μmol, 1.00 eq) in DMF (2 mL) were added aq. HCHO (209 mg, 2.57 mmol, 191 μL, 37.0% purity, 6.21 eq), AcOH (1.50 g, 25.0 mmol, 1.43 mL, 60.3 eq) and NaBH(OAc)₃ (439 mg, 2.07 mmol, 5.00 eq). The mixture was stirred at 25° C. for 2 h. LCMS showed the desired compound was detected. The reaction mixture was concentrated. The residue was purified by prep-HPLC (column: Waters Xbridge BEH C18 100*30 mm*10 um; mobile phase: [water (NH₄HCO₃)-ACN]; B %: 35%-65%, 8 min) to give the title compound (Compound 35) (19.8 mg, 39.8 μmol, 9.62% yield, 100% purity) as a white solid. LCMS (ES⁺): m/z 497.1 [M+H]⁺.

### Example 36. Synthesis of 6-(5-((4-([1, 1′-biphenyl]-3-yl)-5-chloropyrimidin-2-yl)amino)pyridin-3-yl)-2-acetyl-2,6-diazaspiro[3.4]octan-7-one (Compound 36)

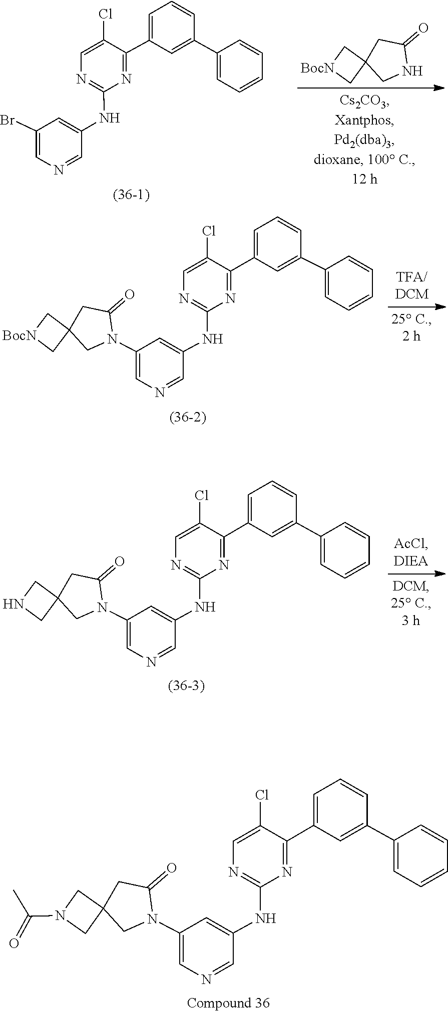 [SMILES: Clc1cnc(Nc2cncc(Br)c2)nc1-c1cccc(-c2ccccc2)c1 ; CC(C)(C)OC(=O)N1CC2(CNC(=O)C2)C1 ; CC(C)(C)OC(=O)N1CC2(CC(=O)N(c3cncc(Nc4ncc(Cl)c(-c5cccc(-c6ccccc6)c5)n4)c3)C2)C1 ; O=C1CC2(CNC2)CN1c1cncc(Nc2ncc(Cl)c(-c3cccc(-c4ccccc4)c3)n2)c1 ; CC(=O)N1CC2(CC(=O)N(c3cncc(Nc4ncc(Cl)c(-c5cccc(-c6ccccc6)c5)n4)c3)C2)C1]

Synthesis of tert-butyl 6-(5-((4-([1,1′-biphenyl]-3-yl)-5-chloropyrimidin-2-yl)amino)pyridin-3-yl)-7-oxo-2,6-diazaspiro[3.4]octane-2-carboxylate (36-2). To a stirred solution of 4-([1,1′-biphenyl]-3-yl)-N-(5-bromopyridin-3-yl)-5-chloropyrimidin-2-amine (36-1) (500 mg, 1.14 mmol, 1.00 eq) and tert-butyl 6-oxo-2,7-diazaspiro[3.4]octane-2-carboxylate (284 mg, 1.26 mmol, 1.10 eq) in dioxane (20 mL) were added Xantphos (132 mg, 228 μmol, 0.200 eq), Pd₂(dba)₃ (105 mg, 114 μmol, 0.100 eq) and Cs₂CO₃ (930 mg, 2.86 mmol, 2.50 eq). The mixture was stirred at 100° C. for 12 h under N₂. LCMS showed the desired compound was detected. The reaction mixture was concentrated. The residue was purified by column chromatography (SiO₂, Petroleum ether:Ethyl acetate=100:1 to 0:1) to give the title compound (36-2) (530 mg, 805 μmol, 70.5% yield, 88.5% purity) as an orange solid. LCMS (ES⁺): m/z 583.2 [M+H]⁺.

Synthesis of 6-(5-((4-([1,1′-biphenyl]-3-yl)-5-chloropyrimidin-2-yl)amino)pyridin-3-yl)-2,6-diazaspiro[3.4]octan-7-one (36-3). To a stirred solution of tert-butyl 6-(5-((4-([1,1′-biphenyl]-3-yl)-5-chloropyrimidin-2-yl)amino)pyridin-3-yl)-7-oxo-2,6-diazaspiro[3.4]octane-2-carboxylate (36-2) (520 mg, 892 μmol, 1.00 eq) in DCM (26 mL) was added TFA (8.01 g, 70.2 mmol, 5.20 mL, 78.8 eq). The mixture was stirred at 25° C. for 2 h. LCMS showed the desired compound was detected. The reaction mixture was concentrated to give the title compound (36-3) (500 mg, 834 μmol, 93.5% yield, 80.5% purity) as a brown oil, and it was used into the next step without further purification. LCMS (ES⁺): m/z 483.2 [M+H]⁺.

Synthesis of 6-(5-((4-([1,1′-biphenyl]-3-yl)-5-chloropyrimidin-2-yl)amino)pyridin-3-yl)-2-acetyl-2,6-diazaspiro[3.4]octan-7-one (Compound 36). To a stirred solution of 6-(5-((4-([1,1′-biphenyl]-3-yl)-5-chloropyrimidin-2-yl)amino)pyridin-3-yl)-2,6-diazaspiro[3.4]octan-7-one (36-3) (200 mg, 414 μmol, 1.00 eq) in DCM (2 mL) were added DIEA (107 mg, 828 μmol, 144 μL, 2.00 eq) and Acetyl Chloride (32.5 mg, 414 μmol, 29.6 μL, 1.00 eq). The mixture was stirred at 25° C. for 3 h. LCMS showed the desired compound was detected. The reaction mixture was concentrated. The residue was purified by prep-HPLC (column: Phenomenex luna C18 80*40 mm*3 um; mobile phase: [water (FA)-ACN]; B %: 20%-60%, 8 min) to give the title compound (Compound 36) (30.4 mg, 57.9 μmol, 14.0% yield, 100% purity) as a white solid. LCMS (ES⁺): m/z 525.1 [M+H]⁺.

### Example 37. Synthesis of 1-(5-((4-([1,3′-bipyrrolidin]-1′-yl)-5-chloropyrimidin-2-yl)amino) pyridin-3-yl)pyrrolidin-2-one (Compound 37)

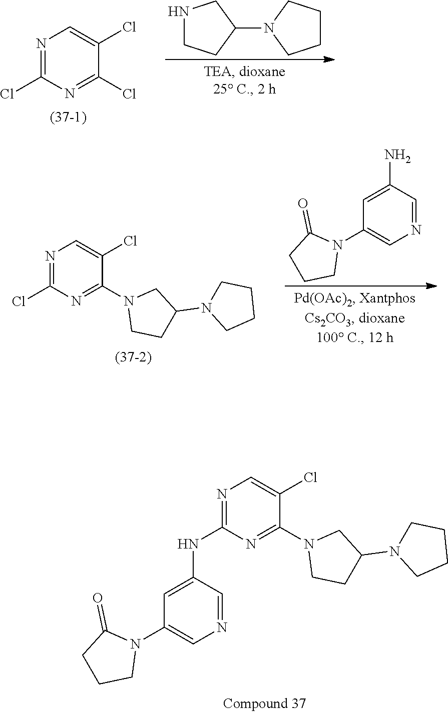 [SMILES: Clc1ncc(Cl)c(Cl)n1 ; C1CCN(C2CCNC2)C1 ; Clc1ncc(Cl)c(N2CCC(N3CCCC3)C2)n1 ; Nc1cncc(N2CCCC2=O)c1 ; O=C1CCCN1c1cncc(Nc2ncc(Cl)c(N3CCC(N4CCCC4)C3)n2)c1]

Synthesis of 1′-(2,5-dichloropyrimidin-4-yl)-1,3′-bipyrrolidine (37-2). To a solution of 2,4,5-trichloropyrimidine (37-1) (262 mg, 1.43 mmol, 1.00 eq) and TEA (433 mg, 4.28 mmol, 596 μL, 3.00 eq) in dioxane (3 mL) was added 1-pyrrolidin-3-ylpyrrolidine (200 mg, 1.43 mmol, 1.00 eq). The mixture was stirred at 25° C. for 2 h. LCMS showed 2,4,5-trichloropyrimidine (37-1) was consumed completely and desired mass was detected. The reaction mixture was concentrated under reduced pressure to get a residue. The residue was partitioned between H₂O (10 mL) and EtOAc (10 mL). The organic phase was separated, dried over Na₂SO₄, filtered and concentrated under reduced pressure to give the title compound (37-2) (300 mg, 1.04 mmol, 73.2% yield) as a yellow solid, and it was used into the next step without further purification. LCMS (ES⁺): m/z 287.0 [M+H]⁺.

Synthesis of 1-(5-((4-([1,3′-bipyrrolidin]-1′-yl)-5-chloropyrimidin-2-yl)amino)pyridin-3-yl)pyrrolidin-2-one (Compound 37). To a solution of 1′-(2,5-dichloropyrimidin-4-yl)-1,3′-bipyrrolidine (37-2) (150 mg, 522 μmol, 0.800 eq), 1-(5-amino-3-pyridyl)pyrrolidin-2-one (116 mg, 653 μmol, 1.00 eq), Cs₂CO₃ (638 mg, 1.96 mmol, 3.00 eq) and Xantphos (75.6 mg, 131 μmol, 0.200 eq) in dioxane (3 mL) was added Pd(OAc)₂ (14.7 mg, 65.3 μmol, 0.100 eq). The mixture was stirred at 100° C. for 0.5 h under N₂. LCMS showed 1′-(2,5-dichloropyrimidin-4-yl)-1,3′-bipyrrolidine (37-2) was consumed completely and desired mass was detected. The reaction mixture was filtered and the filtrate was concentrated under reduced pressure to remove solvent. The residue was purified by prep-HPLC (column: Phenomenex luna C18 80*40 mm*3 um; mobile phase: [water (FA)-ACN]; B %: 1%-35%, 8 min) to give the title compound (Compound 37) (17.2 mg, 40.2 μmol, 6.16% yield, 100% purity) as a white solid. LCMS (ES⁺): m/z 428.1 [M+H]⁺.

### Example 38. Synthesis of 1-(5-((5-chloro-4-(3-(piperidin-1-yl)phenyl)pyrimidin-2-yl)amino)pyridin-3-yl)pyrrolidin-2-one (Compound 38)

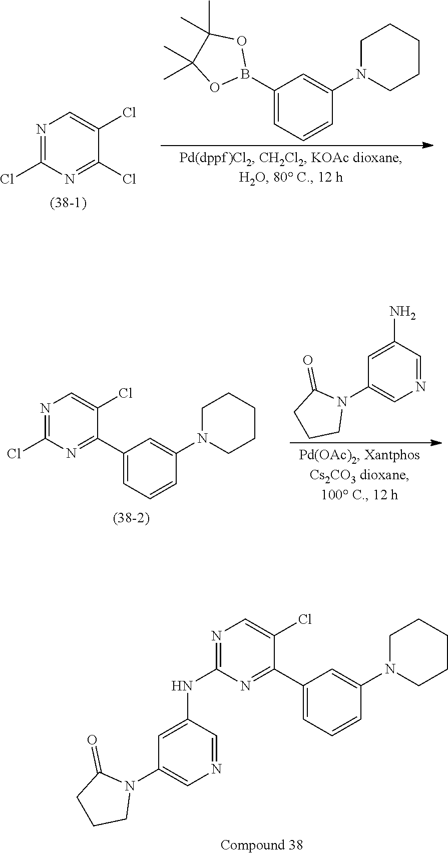

Synthesis of 2,5-dichloro-4-(3-(piperidin-1-yl)phenyl)pyrimidine (38-2). A mixture of 2,4,5-trichloropyrimidine (38-1) (250 mg, 1.36 mmol, 1.00 eq), 1-[3-(4,4,5,5-tetramethyl-1,3,2-dioxaborolan-2-yl)phenyl]piperidine (509 mg, 1.77 mmol, 1.30 eq), KOAc (401 mg, 4.09 mmol, 3.00 eq), Pd(dppf)Cl₂·CH₂Cl₂ (77.9 mg, 95.4 μmol, 0.0700 eq) in dioxane (9 mL) and H₂O (3 mL) was degassed and purged with N₂ for 3 times, and then the mixture was stirred at 80° C. for 12 h under N₂ atmosphere. LCMS showed several new peaks were shown on LCMS and desired compound was detected. The reaction mixture was concentrated under reduced pressure to remove solvent. The residue was diluted with H₂O (20 mL) and extracted with DCM (10 mL*3). The combined organic layers were dried over Na₂SO₄, filtered and concentrated under reduced pressure to give the title compound (38-2) (380 mg, crude) as a black oil. LCMS (ES⁺): m/z 308.1 [M+H]⁺.

Synthesis of 1-(5-((5-chloro-4-(3-(piperidin-1-yl)phenyl)pyrimidin-2-yl)amino)pyridin-3-yl)pyrrolidin-2-one (Compound 38). To a solution of 2,5-dichloro-4-(3-(piperidin-1-yl)phenyl)pyrimidine (38-2) (300 mg, 973 μmol, 1.00 eq) and 1-(5-amino-3-pyridyl)pyrrolidin-2-one (172 mg, 973 μmol, 1.00 eq) in dioxane (17 mL) were added Cs₂CO₃ (951 mg, 2.92 mmol, 3.00 eq), Pd(OAc)₂ (21.85 mg, 97.34 μmol, 0.100 eq) and Xantphos (113 mg, 195 μmol, 0.200 eq). The mixture was stirred at 100° C. for 12 h under N₂. LCMS showed several new peaks and the desired compound was detected. The reaction mixture was concentrated under reduced pressure to remove solvent to give a residue. The crude product was purified by prep-HPLC (column: Waters Xbridge Prep OBD C18 150*40 mm*10 um; mobile phase: [water (NH₄HCO₃)-ACN]; B %: 40%-70%, 8 min) to give the title compound (Compound 38) (42.5 mg, 94.7 μmol, 9.73% yield, 95.6% purity) as a white solid. LCMS (ES⁺): m/z 448.2 [M+H]⁺.

### Example 39. Synthesis of 1-(5-((5-chloro-4-(3-cyclobutylphenyl)pyrimidin-2-yl)amino)pyridin-3-yl)pyrrolidin-2-one (Compound 39)

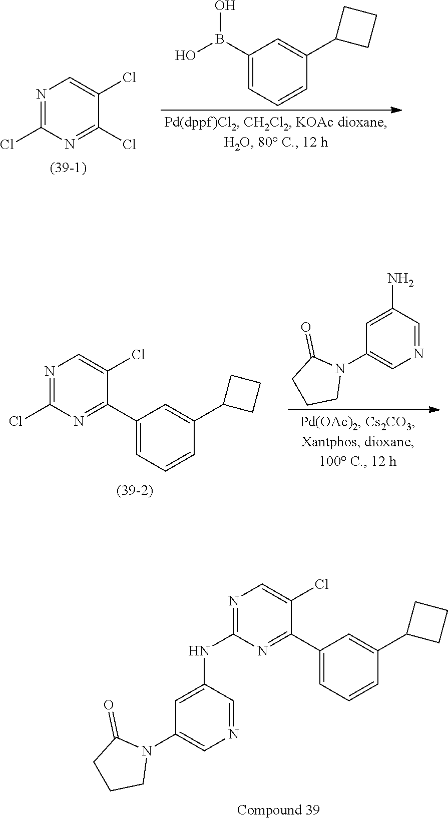

Synthesis of 2, 5-dichloro-4-(3-cyclobutylphenyl)pyrimidine (39-2). To a solution of 2,4,5-trichloropyrimidine (39-1) (240 mg, 1.31 mmol, 1.00 eq) and (3-cyclobutylphenyl)boronic acid (300 mg, 1.70 mmol, 1.30 eq) in dioxane (9 mL) and H₂O (3 mL) were added Pd(dppf)Cl₂·CH₂Cl₂ (75.0 mg, 91.8 μmol, 0.0700 eq) and KOAc (386 mg, 3.93 mmol, 3.00 eq). The mixture was stirred at 80° C. for 12 h under N₂. LCMS showed several new peaks and desired compound was detected. The reaction mixture was concentrated under reduced pressure to remove solvent to give the title compound (39-2) (320 mg, crude) as a white solid. LCMS (ES⁺): m/z 279.0 [M+H]⁺.

Synthesis of 1-(5-((5-chloro-4-(3-cyclobutylphenyl)pyrimidin-2-yl)amino)pyridin-3-yl)pyrrolidin-2-one (Compound 39). To a solution of 2,5-dichloro-4-(3-cyclobutylphenyl)pyrimidine (39-2) (300 mg, 1.07 mmol, 1.00 eq) and 1-(5-amino-3-pyridyl)pyrrolidin-2-one (190 mg, 1.07 mmol, 1.00 eq) in dioxane (17 mL) were added Cs₂CO₃ (1.05 g, 3.22 mmol, 3.00 eq), Pd(OAc)₂ (24.1 mg, 107 μmol, 0.100 eq) and Xantphos (124 mg, 215 μmol, 0.200 eq). The mixture was stirred at 100° C. for 12 h under N₂. LCMS showed several new peaks and desired compound was detected. The reaction mixture was concentrated under reduced pressure to remove solvent to give a residue. The crude product was purified by reversed-phase MPLC (0.1% FA condition) to give the title compound (Compound 39) (17.5 mg, 41.7 μmol, 3.88% yield) as a white solid. LCMS (ES⁺): m/z 420.2 [M+H]⁺.

### Example 40. Synthesis of 1-(5-((5-chloro-4-(3-cyclopentylphenyl)pyrimidin-2-yl)amino)pyridin-3-yl)pyrrolidin-2-one (Compound 40)

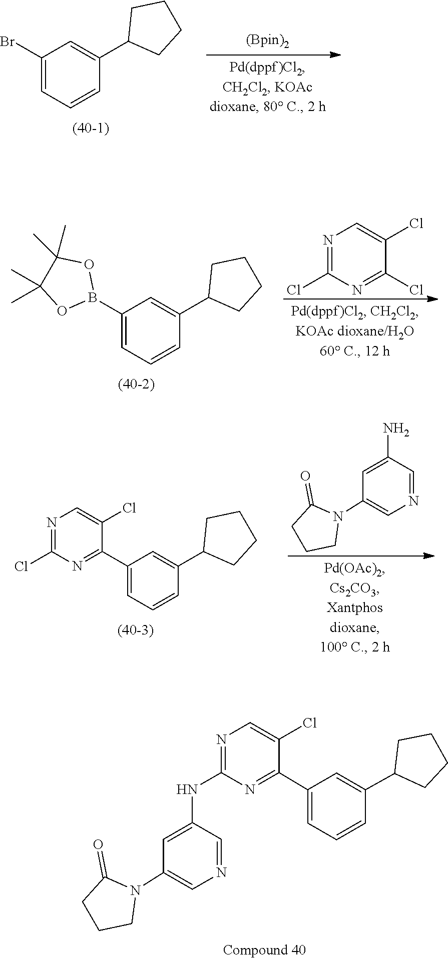

Synthesis of 2-(3-cyclopentylphenyl)-4,4,5,5-tetramethyl-1,3,2-dioxaborolane (40-2). To a stirred solution of 1-bromo-3-cyclopentylbenzene (40-1) (300 mg, 1.33 mmol, 1.00 eq) and 4,4,5,5-tetramethyl-2-(4,4,5,5-tetramethyl-1,3,2-dioxaborolan-2-yl)-1,3,2-dioxaborolane (508 mg, 2.00 mmol, 1.50 eq) in Dioxane (5 mL) were added Pd(dppf)Cl₂·CH₂Cl₂ (109 mg, 133 μmol, 0.100 eq) and KOAc (392 mg, 4.00 mmol, 3.00 eq). The mixture was stirred at 80° C. for 2 h. TLC (Petroleum ether:Ethyl acetate=10:1, Rf=0.2) indicated 1-bromo-3-cyclopentylbenzene (40-1) remained and one new spot 2-(3-cyclopentylphenyl)-4,4,5,5-tetramethyl-1,3,2-dioxaborolane (40-2) formed. The reaction mixture was concentrated to give the title compound (40-2) (550 mg, crude) as a dark brown oil, and it was used into the next step without further purification.

Synthesis of 2,5-dichloro-4-(3-cyclopentylphenyl)pyrimidine (40-3). To a stirred solution of 2-(3-cyclopentylphenyl)-4,4,5,5-tetramethyl-1,3,2-dioxaborolane (40-2) (350 mg, 1.29 mmol, 1.30 eq) and 2,4,5-trichloropyrimidine (181 mg, 989 μmol, 1.00 eq) in dioxane (4.5 mL) and water (1.5 mL) were added Pd(dppf)Cl₂·CH₂Cl₂ (56.5 mg, 69.2 μmol, 0.0700 eq) and KOAc (291 mg, 2.97 mmol, 3.00 eq). The mixture was stirred at 60° C. for 12 h. LCMS showed the desired compound was detected. The reaction mixture was concentrated under reduced pressure to remove solvent. The residue was diluted with H₂O (5 mL) and extracted with EtOAc (20 mL*3). The combined organic layers were washed with water (10 mL*3), dried over Na₂SO₄, filtered and concentrated under reduced pressure to give the title compound (40-3) (1.00 g, crude) as a dark brown oil, and it was used into the next step without further purification. LCMS (ES⁺): m/z 293.1 [M+H]⁺.

Synthesis of 1-(5-((5-chloro-4-(3-cyclopentylphenyl)pyrimidin-2-yl)amino)pyridin-3-yl)pyrrolidin-2-one (Compound 40). To a stirred solution of 2,5-dichloro-4-(3-cyclopentylphenyl)pyrimidine (40-3) (200 mg, 682 μmol, 1.00 eq) and 1-(5-amino-3-pyridyl)pyrrolidin-2-one (133 mg, 750 μmol, 1.10 eq) in dioxane (3 mL) were added Xantphos (78.9 mg, 136 μmol, 0.200 eq), Pd(OAc)₂ (15.3 mg, 68.2 μmol, 0.100 eq) and Cs₂CO₃ (556 mg, 1.71 mmol, 2.50 eq). The mixture was stirred at 100° C. for 2 h under N₂. LCMS showed the desired compound was detected. The reaction mixture was concentrated. The residue was purified by prep-HPLC (column: Waters Xbridge Prep OBD C18 150*40 mm*10 um; mobile phase: [water (NH₄HCO₃)-ACN]; B %: 45%-80%, 8 min) to give the title compound (Compound 40) (9.90 mg, 22.8 μmol, 3.34% yield, 100% purity) as a white solid. LCMS (ES⁺): m/z 432.2 [M−H]⁺

### Example 41. Synthesis of 1-[5-[[5-chloro-4-(2-phenylmorpholin-4-yl)pyrimidin-2-yl]amino]-3-pyridyl]pyrrolidin-2-one (Compound 41)

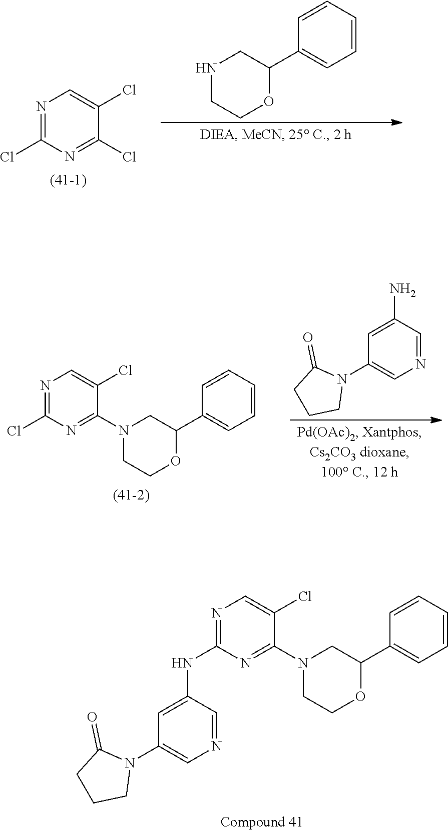

Synthesis of 4-(2,5-dichloropyrimidin-4-yl)-2-phenyl-morpholine (41-2). To a solution of 2,4,5-trichloropyrimidine (41-1) (1.00 g, 5.45 mmol, 1.00 eq) in MeCN (10 mL) were added DIEA (1.41 g, 10.9 mmol, 1.90 mL, 2.00 eq) and 2-phenylmorpholine (979 mg, 6.00 mmol, 1.10 eq) at 25° C. The mixture was stirred at 25° C. for 2 h. LCMS showed 2,4,5-trichloropyrimidine (41-1) was remaining and desired mass was detected. The reaction mixture was diluted with H₂O (10 mL) and extracted with EtOAc (20 mL*3). The combined organic layers were dried over Na₂SO₄, filtered and concentrated under reduced pressure. The residue was purified by column chromatography (SiO₂, Petroleum ether:Ethyl acetate=30:1 to 20:1) to give the title compound (41-2) (1.46 g, 4.66 mmol, 85.5% yield, 99.0% purity) as a white solid. LCMS (ES⁺): m/z 310.0 [M+H]⁺.

Synthesis of 1-[5-[[5-chloro-4-(2-phenylmorpholin-4-yl)pyrimidin-2-yl]amino]-3-pyridyl]pyrrolidin-2-one (Compound 41). A mixture of 4-(2,5-dichloropyrimidin-4-yl)-2-phenyl-morpholine (41-2) (200 mg, 645 μmol, 1.00 eq), 1-(5-amino-3-pyridyl)pyrrolidin-2-one (160 mg, 903 μmol, 1.40 eq), Xantphos (74.6 mg, 129 μmol, 0.200 eq), Pd(OAc)₂ (29.0 mg, 129 μmol, 0.200 eq) and Cs₂CO₃ (630 mg, 1.93 mmol, 3.00 eq) in dioxane (3 mL) was degassed and purged with N₂ for 3 times at 25° C., and then the mixture was stirred at 100° C. for 12 h under N₂ atmosphere. LCMS showed 4-(2,5-dichloropyrimidin-4-yl)-2-phenyl-morpholine (41-2) was consumed completely and desired mass was detected. The reaction mixture was diluted with H₂O (10 mL) and extracted with EtOAc (10 mL*3). The combined organic layers were dried over Na₂SO₄, filtered and concentrated under reduced pressure. The residue was purified by prep-HPLC (column: Phenomenex luna C18 80*40 mm*3 um; mobile phase: [water (FA)-ACN]; B %: 20%-60%, 8 min) to give the title compound (Compound 41) (58.5 mg, 126 μmol, 19.6% yield, 97.5% purity) as a yellow solid. LCMS (ES⁺): m/z 451.2 [M+H]⁺.

### Example 42. Synthesis of 1-(5-((5-chloro-4-(3-phenylpyrrolidin-1-yl)pyrimidin-2-yl)amino)pyridin-3-yl)pyrrolidin-2-one (Compound 42)

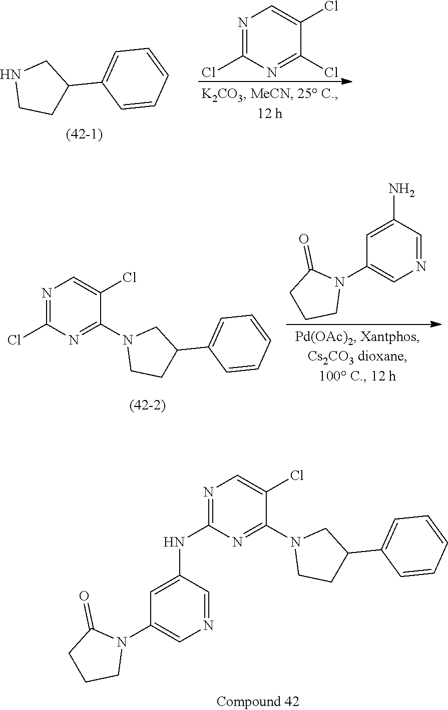

Synthesis of 2,5-dichloro-4-(3-phenylpyrrolidin-1-yl)pyrimidine 42-2. To a solution of 3-phenylpyrrolidine (42-1) (1.02 g, 5.56 mmol, 1.00 eq) and 2,4,5-trichloropyrimidine (900 mg, 6.11 mmol, 1.10 eq) in MeCN (20 mL) was added K₂CO₃ (2.30 g, 16.7 mmol, 3.00 eq) at 25° C. The mixture was stirred at 25° C. for 12 h. LC-MS showed desired mass was detected. The reaction mixture was diluted with H₂O (10 mL) and extracted with EtOAc (10 mL*3). The combined organic layers were dried over Na₂SO₄, filtered and concentrated under reduced pressure. The residue was purified by column chromatography (SiO₂, Petroleum ether:Ethyl acetate=1:0 to 5:1) to give the title compound (42-2) (1.40 g, 4.28 mmol, 77.1% yield, 90.0% purity) as a white solid. LCMS (ES⁺): m/z 294.1 [M+H]⁺.

Synthesis of 1-(5-((5-chloro-4-(3-phenylpyrrolidin-1-yl)pyrimidin-2-yl)amino)pyridin-3-yl)pyrrolidin-2-one (Compound 42). To a solution of 2,5-dichloro-4-(3-phenylpyrrolidin-1-yl)pyrimidine (42-2) (50.0 mg, 170 μmol, 1.00 eq) and 1-(5-amino-3-pyridyl)pyrrolidin-2-one (42.2 mg, 238 μmol, 1.40 eq) in dioxane (5 mL) were added Cs₂CO₃ (277 mg, 850 μmol, 5.00 eq) and Pd(OAc)₂ (7.63 mg, 34.0 μmol, 0.200 eq) at 25° C., then Xantphos (19.7 mg, 34.0 μmol, 0.200 eq) was added under N₂. The mixture was stirred at 100° C. for 12 h. LCMS showed desired mass was detected. The reaction mixture was diluted with H₂O (10 mL) and extracted with EtOAc (10 mL*3). The combined organic layers were dried over Na₂SO₄, filtered and concentrated under reduced pressure. The residue was purified by prep-HPLC (column: Phenomenex luna C18 80*40 mm*3 um; mobile phase: [water (FA)-ACN]; B %: 20%-60%, 8 min) to give the title compound (Compound 42) (50.2 mg, 114 μmol, 67.1% yield, 96.8% purity) as a white solid. LCMS (ES⁺): m/z 435.2 [M+H]⁺.

### Example 43. Synthesis of (S)-1-(5-((5-chloro-4-(3-phenylpiperidin-1-yl)pyrimidin-2-yl)amino)pyridin-3-yl)pyrrolidin-2-one (Compound 43)

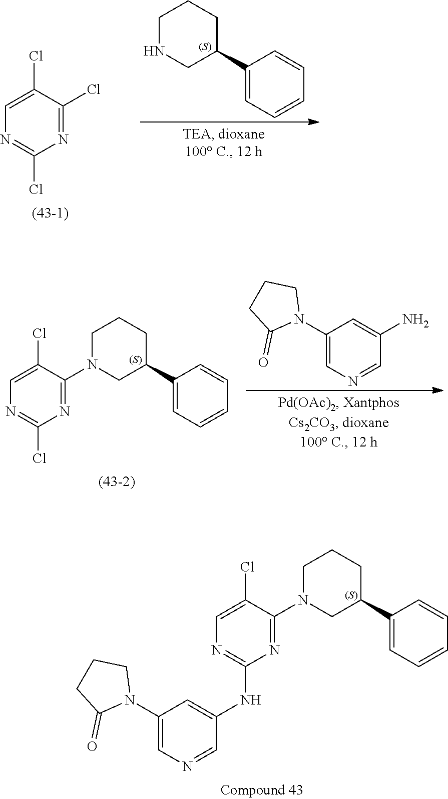

Synthesis of (S)-2,5-dichloro-4-(3-phenylpiperidin-1-yl)pyrimidine (43-2). To a solution of 2,4,5-trichloropyrimidine (43-1) (284 mg, 1.55 mmol, 1.00 eq) in dioxane (10 mL) were added TEA (173 mg, 1.71 mmol, 237 μL, 1.10 eq) and 3-phenylpiperidine (300 mg, 1.86 mmol, 1.20 eq) at 25° C. The mixture was stirred at 100° C. for 12 h. LCMS showed 2,4,5-trichloropyrimidine (43-1) was consumed completely and the desired mass was detected. The reaction mixture was concentrated under reduced pressure to remove solvent. The residue was purified by prep-TLC (SiO₂, Petroleum ether:Ethyl acetate=10:1) to give the title compound 43-2 (331 mg, 1.07 mmol, 69.3% yield) as a white solid. LCMS (ES⁺): m/z 308.1 [M+H]⁺.

Synthesis of (S)-1-(5-((5-chloro-4-(3-phenylpiperidin-1-yl)pyrimidin-2-yl)amino)pyridin-3-yl)pyrrolidin-2-one (Compound 43). To a solution of (S)-2,5-dichloro-4-(3-phenylpiperidin-1-yl)pyrimidine (43-2) (210 mg, 681 μmol, 1.00 eq) in dioxane (5 mL) were added 1-(5-amino-3-pyridyl)pyrrolidin-2-one (173 mg, 681 μmol, 70.0% purity, 1.00 eq), Xantphos (11.8 mg, 20.4 μmol, 0.0300 eq), Cs₂CO₃ (444 mg, 1.36 mmol, 2.00 eq) and Pd(OAc)₂ (7.65 mg, 34.1 μmol, 0.0500 eq) at 25° C. under N₂. The mixture was stirred at 100° C. for 12 h under N₂ atmosphere. LCMS showed (S)-2,5-dichloro-4-(3-phenylpiperidin-1-yl)pyrimidine (43-2) was consumed completely and the desired mass was detected. The reaction mixture was concentrated under reduced pressure to remove solvent. The residue was purified by prep-HPLC (column: Phenomenex Luna C18 75*30 mm*3 um; mobile phase: [water (TFA)-ACN]; B %: 25%-55%, 8 min) to give the title compound (Compound 43) (72.3 mg, 148 μmol, 21.8% yield, 92.2% purity) as a yellow solid. LCMS (ES⁺): m/z 449.1 [M+H]⁺.

### Example 44. Synthesis of (R)-1-(5-((5-chloro-4-(3-phenylpiperidin-1-yl)pyrimidin-2-yl)amino)pyridin-3yl)pyrrolidin-2-one (Compound 44)

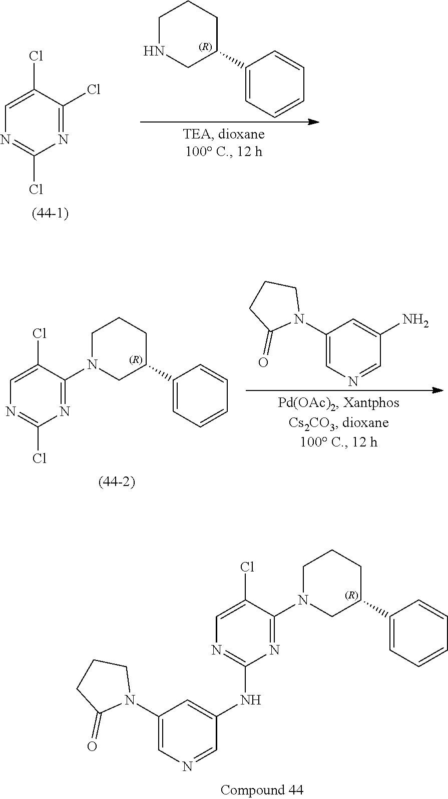

Synthesis of (R)-2,5-dichloro-4-(3-phenylpiperidin-1-yl)pyrimidine 44-2. To a solution of 2,4,5-trichloropyrimidine (44-1) (171 mg, 930 μmol, 1.00 eq) in dioxane (3 mL) were added (3R)-3-phenylpiperidine (150 mg, 930 μmol, 1.00 eq) and TEA (104 mg, 1.02 mmol, 142 μL, 1.10 eq) at 25° C. The mixture was stirred at 100° C. for 12 h. LCMS showed 2,4,5-trichloropyrimidine (44-1) was consumed completely and the desired mass was detected. The reaction mixture was concentrated under reduced pressure to remove solvent. The residue was purified by prep-TLC (SiO₂, Petroleum ether:Ethyl acetate=10:1) to give the title compound (44-2) (280 mg, 908 μmol, 97.7% yield) as a white solid. LCMS (ES⁺): m/z 308.1 [M+H]⁺.

Synthesis of (R)-1-(5-((5-chloro-4-(3-phenylpiperidin-1-yl)pyrimidin-2-yl)amino)pyridin-3yl)pyrrolidin-2-one (Compound 44). To a solution of 1-(5-aminopyridin-3-yl)pyrrolidin-2-one (115 mg, 649 μmol, 1.00 eq) in dioxane (5 mL) were added (R)-2,5-dichloro-4-(3-phenylpiperidin-1-yl)pyrimidine (44-2) (200 mg, 649 μmol, 1.00 eq), Xantphos (11.3 mg, 19.5 μmol, 0.0300 eq), Cs₂CO₃ (423 mg, 1.30 mmol, 2.00 eq) and Pd(OAc)₂ (7.28 mg, 32.5 μmol, 0.0500 eq) at 25° C. under N₂ atmosphere. The mixture was stirred at 110° C. for 12 h. LCMS showed (R)-2,5-dichloro-4-(3-phenylpiperidin-1-yl)pyrimidine (44-2) was consumed completely and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 44) (43.7 mg, 91.7 μmol, 14.1% yield, 94.2% purity) as a white solid. LCMS (ES⁺): m/z 449.0 [M+H]⁺.

### Example 45. Synthesis of 1-(5-((5-chloro-4-(3-cyclohexylpyrrolidin-1-yl)pyrimidin-2-yl)amino)pyridin-3-yl)pyrrolidin-2-one (Compound 45)

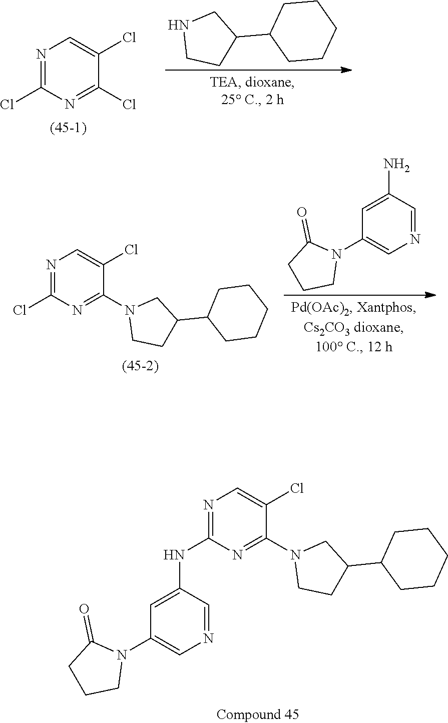

Synthesis of 2,5-dichloro-4-(3-cyclohexylpyrrolidin-1-yl)pyrimidine (45-2). To a solution of 2,4,5-trichloropyrimidine (45-1) (450 mg, 2.45 mmol, 1.60 eq) and TEA (465 mg, 4.60 mmol, 640 μL, 3.00 eq) in dioxane (3 mL) was added 3-cyclohexylpyrrolidine (235 mg, 1.53 mmol, 1.00 eq). The mixture was stirred at 25° C. for 2 h. LCMS showed 2,4,5-trichloropyrimidine 45-1 was consumed completely and desired mass was detected. The reaction mixture was concentrated under reduced pressure to get a residue. The residue was purified by flash silica gel chromatography (ISCO®; 10 g SepaFlash® Silica Flash Column, Eluent of 0-20% Ethyl acetate/Petroleum ether gradient @80 mL/min) to give the title compound (45-2) (460 mg, 1.53 mmol, 99.9% yield) as a white solid. LCMS (ES⁺): m/z 300.1 [M+H]⁺.

Synthesis of 1-(5-((5-chloro-4-(3-cyclohexylpyrrolidin-1-yl)pyrimidin-2-yl)amino)pyridin-3-yl)pyrrolidin-2-one (Compound 45). To a solution of 2,5-dichloro-4-(3-cyclohexylpyrrolidin-1-yl)pyrimidine (45-2) (203 mg, 677 μmol, 0.800 eq), 1-(5-amino-3-pyridyl) pyrrolidin-2-one (150 mg, 846 μmol, 1.00 eq), Cs₂CO₃ (827 mg, 2.54 mmol, 3.00 eq) and Xantphos (98.0 mg, 169 μmol, 0.200 eq) in dioxane (3 mL) was added Pd(OAc)₂ (19.0 mg, 84.7 μmol, 0.100 eq). The mixture was stirred at 100° C. for 12 h under N₂. LCMS showed 2,5-dichloro-4-(3-cyclohexylpyrrolidin-1-yl)pyrimidine (45-2) was consumed completely and desired mass was detected. The reaction mixture was filtered and the filtrate was concentrated under reduced pressure to remove solvent. The residue was purified by prep-HPLC (column: Phenomenex Luna 80*30 mm*3 um; mobile phase: [water (FA)-ACN]; B %: 10%-45%, 8 min) to give the title compound (Compound 45) (86.5 mg, 187 μmol, 22.1% yield, 95.3% purity) as a white solid. LCMS (ES⁺): m/z 441.1 [M+H]⁺.

### Example 46. Synthesis of 1-(5-((5-chloro-4-(3-(pyrrolidin-1-yl)phenyl)pyrimidin-2-yl)amino)pyridin-3-yl)pyrrolidin-2-one (Compound 46)

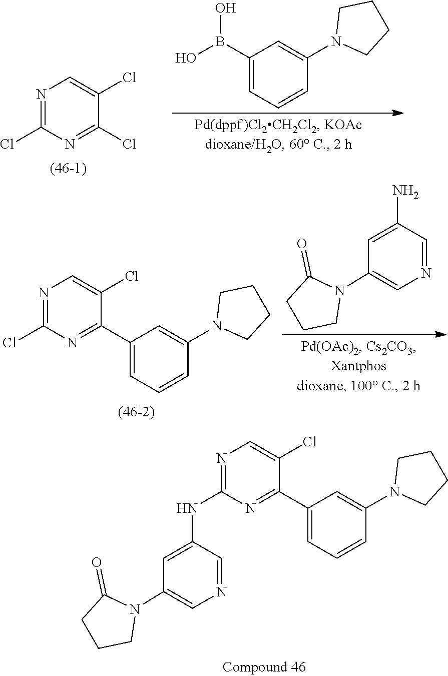

Synthesis of 2, 5-dichloro-4-(3-(pyrrolidin-1-yl)phenyl)pyrimidine (46-2). To a stirred solution of 2,4,5-trichloropyrimidine (46-1) (259 mg, 1.41 mmol, 1.00 eq) and (3-pyrrolidin-1-ylphenyl)boronic acid (350 mg, 1.83 mmol, 1.30 eq) in Dioxane (3 mL) and water (1 mL) were added Pd(dppf)Cl₂·CH₂Cl₂ (80.6 mg, 98.7 μmol, 0.0700 eq) and KOAc (415 mg, 4.23 mmol, 3.00 eq) at 20° C. The mixture was stirred at 60° C. for 2 h. LCMS showed the desired compound was detected. The reaction mixture was concentrated. The residue was purified by column chromatography (SiO₂, Petroleum ether:Ethyl acetate=30:1 to 1:1) to give the title compound (46-2) (380 mg, 1.13 mmol, 80.0% yield, 87.3% purity) as a yellow solid. LCMS (ES⁺): m/z 294.0 [M+H]⁺.

Synthesis of 1-(5-((5-chloro-4-(3-(pyrrolidin-1-yl)phenyl)pyrimidin-2-yl)amino)pyridin-3-yl)pyrrolidin-2-one (Compound 46). To a stirred solution of 2,5-dichloro-4-(3-(pyrrolidin-1-yl)phenyl)pyrimidine (46-2) (150 mg, 510 μmol, 1.00 eq) and 1-(5-amino-3-pyridyl)pyrrolidin-2-one (99.4 mg, 561 μmol, 1.10 eq) in Dioxane (3 mL) were added Xantphos (59.0 mg, 102 μmol, 0.200 eq), Pd(OAc)₂ (11.5 mg, 51.0 μmol, 0.100 eq) and Cs₂CO₃ (415 mg, 1.27 mmol, 2.50 eq) at 100° C. The mixture was stirred at 100° C. for 2 h. LCMS showed the desired compound was detected. The reaction mixture was concentrated. The residue was purified by prep-HPLC (column: Waters Xbridge BEH C18 100*30 mm*10 um; mobile phase: [water (NH₄HCO₃)-ACN]; B %: 45%-75%, 8 min) to give the title compound (Compound 46) (3.60 mg, 8.28 μmol, 1.62% yield, 100% purity) as a yellow solid. LCMS (ES⁺): m/z 433.2 [M−H]⁺.

### Example 47. Synthesis of 1-(5-((5-chloro-4-(1-(1-methylpiperidin-4-yl)-1H-pyrazol-4-yl)pyrimidin-2-yl)amino)pyridin-3-yl)pyrrolidin-2-one (Compound 47)

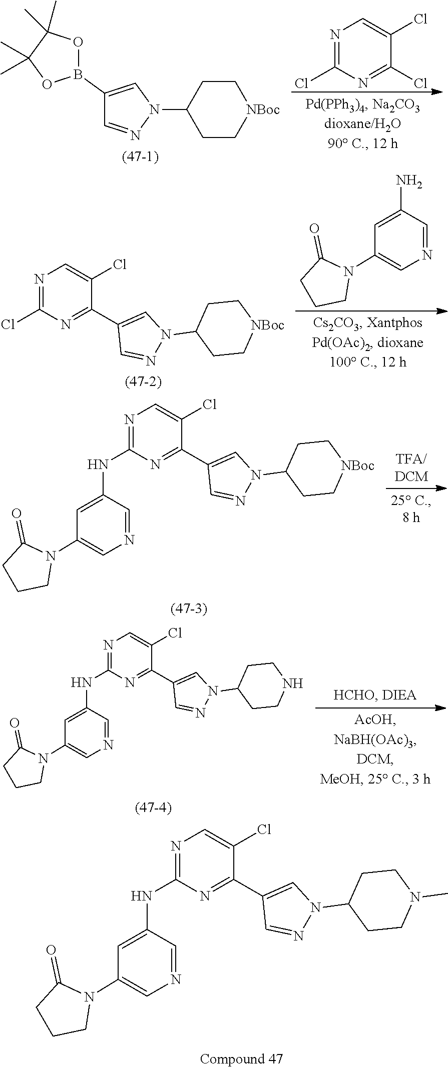

Synthesis of tert-butyl 4-(4-(2,5-dichloropyrimidin-4-yl)-1H-pyrazol-1-yl)piperidine-1-carboxylate (47-2). To a stirred solution of tert-butyl 4-(4-(4,4,5,5-tetramethyl-1,3,2-dioxaborolan-2-yl)-1H-pyrazol-1-yl)piperidine-1-carboxylate (47-1) (3.00 g, 7.95 mmol, 1.00 eq) in dioxane (60 mL) and H₂O (6 mL) were added 2,4,5-trichloropyrimidine (2.92 g, 15.9 mmol, 2.00 eq), Na₂CO₃ (1.69 g, 15.9 mmol, 2.00 eq) and Pd(PPh₃)₄ (184 mg, 159 μmol, 0.0200 eq) at 25° C. under N₂. The mixture was stirred at 90° C. for 12 h under N₂ atmosphere. LCMS showed that tert-butyl 4-(4-(4,4,5,5-tetramethyl-1,3,2-dioxaborolan-2-yl)-1H-pyrazol-1-yl)piperidine-1-carboxylate (47-1) was consumed completely and the desired mass was detected. Water (80 mL) was added to the reaction, the aqueous phase was extracted with EtOAc (80 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 0:1) to give the title compound (47-2) (2.70 g, 6.78 mmol, 85.3% yield) as a yellow solid. LCMS (ES⁺): m/z 342.1 [M+H−56]⁺.

Synthesis of tert-butyl 4-(4-(5-chloro-2-((5-(2-oxopyrrolidin-1-yl)pyridin-3-yl)amino)pyrimidin-4-yl)-1H-pyrazol-1-yl)piperidine-1-carboxylate (47-3). To a solution of tert-butyl 4-(4-(2,5-dichloropyrimidin-4-yl)-1H-pyrazol-1-yl)piperidine-1-carboxylate (47-2) (2.70 g, 6.78 mmol, 0.800 eq) in dioxane (60 mL) were added 1-(5-amino-3-pyridyl)pyrrolidin-2-one (2.36 g, 9.32 mmol, 70.0% purity, 1.10 eq), Pd(OAc)₂ (95.1 mg, 424 μmol, 0.0500 eq), Cs₂CO₃ (5.52 g, 17.0 mmol, 2.00 eq) and Xantphos (147 mg, 254 μmol, 0.0300 eq) at 25° C. under N₂. The mixture was stirred at 100° C. for 12 h under N₂ atmosphere. LCMS showed that tert-butyl 4-(4-(2,5-dichloropyrimidin-4-yl)-1H-pyrazol-1-yl)piperidine-1-carboxylate (47-2) was consumed completely and the desired mass was detected. Water (80 mL) was added to reaction, the aqueous phase was extracted with EtOAc (80 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 0:1) to give the title compound (47-3) (2.20 g, 4.08 mmol, 48.2% yield) as a pink solid. LCMS (ES⁺): m/z 539.2 [M+H]⁺.

Synthesis of 1-(5-((5-chloro-4-(1-(piperidin-4-yl)-1H-pyrazol-4-yl)pyrimidin-2-yl)amino)pyridin-3-yl)pyrrolidin-2-one (47-4). A solution of tert-butyl 4-(4-(5-chloro-2-((5-(2-oxopyrrolidin-1-yl)pyridin-3-yl)amino)pyrimidin-4-yl)-1H-pyrazol-1-yl)piperidine-1-carboxylate (47-3) (1.00 g, 1.86 mmol, 1.00 eq) in TFA (2 mL) and DCM (10 mL) was stirred at 25° C. for 8 h. LCMS showed that tert-butyl 4-(4-(5-chloro-2-((5-(2-oxopyrrolidin-1-yl)pyridin-3-yl)amino)pyrimidin-4-yl)-1H-pyrazol-1-yl)piperidine-1-carboxylate (47-3) was consumed completely and the desired mass was detected. The reaction mixture was concentrated under reduced pressure to give the title compound (47-4) (1.00 g, crude, TFA) as a yellow oil. LCMS (ES⁺): m/z 439.2 [M+H]⁺.

Synthesis of 1-(5-((5-chloro-4-(1-(1-methylpiperidin-4-yl)-1H-pyrazol-4-yl)pyrimidin-2-yl)amino)pyridin-3-yl)pyrrolidin-2-one (Compound 47). To a solution of 1-(5-((5-chloro-4-(1-(piperidin-4-yl)-1H-pyrazol-4-yl)pyrimidin-2-yl)amino)pyridin-3-yl)pyrrolidin-2-one (47-4) (300 mg, 543 μmol, 1.00 eq, TFA) in DCM (4 mL) and MeOH (2 mL) were added aq. HCHO (749 mg, 9.22 mmol, 687 μL, 37.0% purity, 17.0 eq) and DIEA (70.1 mg, 543 μmol, 94.5 μL, 1.00 eq) at 25° C. The mixture was stirred at 25° C. for 0.5 h. AcOH (32.6 mg, 543 μmol, 31.0 μL, 1.00 eq) was added to adjust pH to 5 at 25° C. The mixture was stirred at 25° C. for 0.5 h. NaBH(OAc)₃ (138 mg, 651 μmol, 1.20 eq) was added to the mixture at 25° C. The mixture was stirred at 25° C. for 2 h. LCMS showed that 1-(5-((5-chloro-4-(1-(piperidin-4-yl)-1H-pyrazol-4-yl)pyrimidin-2-yl)amino)pyridin-3-yl)pyrrolidin-2-one (47-4) was consumed completely and the desired mass was detected. The reaction mixture was concentrated under reduced pressure. The residue was purified by prep-HPLC (column: Phenomenex Luna C18 75*30 mm*3 um; mobile phase: [water (FA)-ACN]; B %: 10%-40%, 8 min) to give the title compound (Compound 47) (65.1 mg, 141 μmol, 25.9% yield, 97.8% purity) as a white solid. LCMS (ES⁺): m/z 453.1 [M+H]⁺.

### Example 48. Synthesis of 1-(5-((4-(1-(1-acetylpiperidin-4-yl)-1H-pyrazol-4-yl)-5-chloropyrimidin-2-yl)amino)pyridin-3-yl)pyrrolidin-2-one (Compound 48)

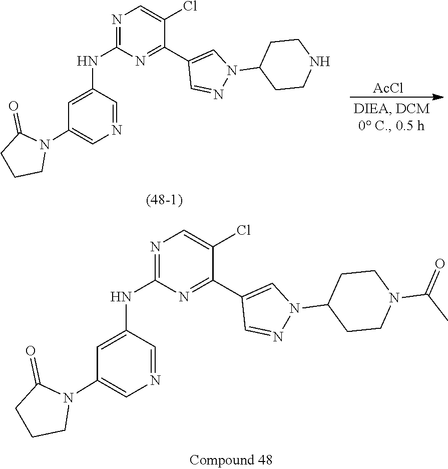 [SMILES: "AcCl" ; O=C1CCCN1c1cncc(Nc2ncc(Cl)c(-c3cnn(C4CCNCC4)c3)n2)c1 ; CC(=O)N1CCC(n2cc(-c3nc(Nc4cncc(N5CCCC5=O)c4)ncc3Cl)cn2)CC1]

Synthesis of 1-(5-((4-(1-(1-acetylpiperidin-4-yl)-1H-pyrazol-4-yl)-5-chloropyrimidin-2-yl)amino)pyridin-3-yl)pyrrolidin-2-one (Compound 48). To a solution of 1-(5-((5-chloro-4-(1-(piperidin-4-yl)-1H-pyrazol-4-yl)pyrimidin-2-yl)amino)pyridin-3-yl)pyrrolidin-2-one (48-1) (prepared as described in Example 47) (300 mg, 543 μmol, 1.00 eq, TFA) in DCM (8 mL) were added AcCl (51.1 mg, 651 μmol, 46.5 μL, 1.20 eq) and DIEA (210 mg, 1.63 mmol, 284 μL, 3.00 eq) at 0° C. The mixture was stirred at 0° C. for 0.5 h. LCMS showed that 1-(5-((5-chloro-4-(1-(piperidin-4-yl)-1H-pyrazol-4-yl)pyrimidin-2-yl)amino)pyridin-3-yl)pyrrolidin-2-one (48-1) was consumed completely and the desired mass was detected. The reaction mixture was concentrated under reduced pressure. The residue was purified by prep-HPLC (column: Phenomenex Luna C18 75*30 mm*3 um; mobile phase: [water (FA)-ACN]; B %: 15%-45%, 8 min) to give the title compound (Compound 48) (75.4 mg, 154 μmol, 28.5% yield, 98.5% purity) as a white solid. LCMS (ES⁺): m/z 481.1 [M+H]⁺.

### Example 49. Synthesis of 1-(5-((5-chloro-4-(5-phenyl-1H-pyrazol-3-yl)pyrimidin-2-yl)amino)pyridin-3-yl)pyrrolidin-2-one (Compound 49)

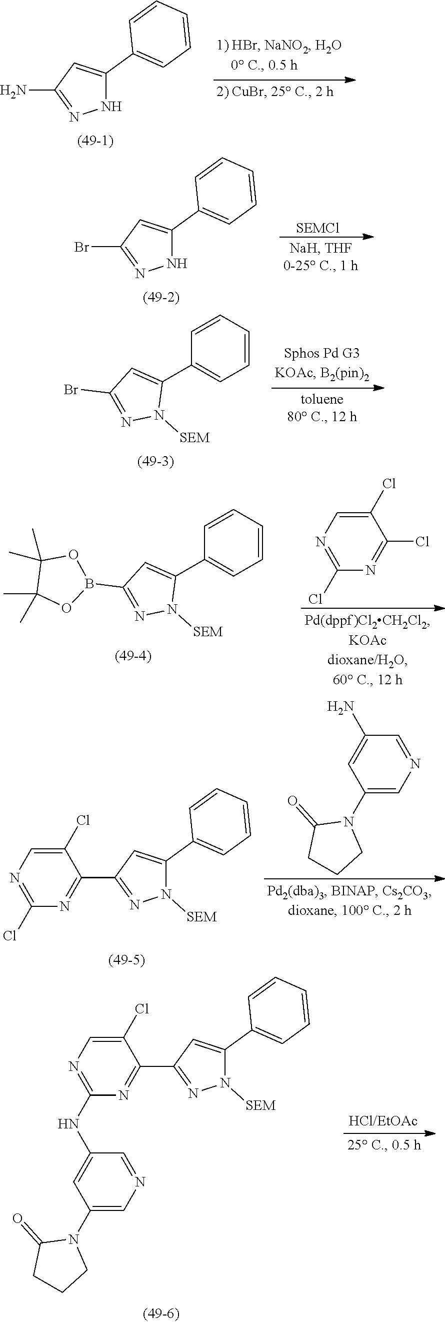

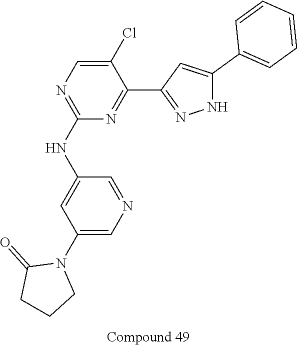 [SMILES: O=C1CCCN1c1cncc(Nc2ncc(Cl)c(-c3cc(-c4ccccc4)[nH]n3)n2)c1]

Synthesis of 3-bromo-5-phenyl-JH-pyrazole (49-2). To a solution of 5-phenyl-1H-pyrazol-3-amine (49-1) (10.0 g, 62.8 mmol, 1.00 eq) and aq. HBr (12.7 g, 62.8 mmol, 8.53 mL, 40.0% purity, 1.00 eq) in H₂O (80 mL) was added NaNO₂ (4.77 g, 69.1 mmol, 1.10 eq) at 0° C. and then stirred at 0° C. for 0.5 h. CuBr (9.91 g, 69.1 mmol, 2.1 mL, 1.10 eq) was added and the mixture was stirred at 25° C. for 2 h. LCMS showed 5-phenyl-1H-pyrazol-3-amine (49-1) was consumed completely and desired mass was detected. The reaction mixture was quenched by the addition of saturated aqueous KOH (10 mL). The reaction mixture was partitioned between H₂O (100 mL) and EtOAc (100 mL). The organic layer was dried over Na₂SO₄, filtered and concentrated in vacuum. The residue was purified by column chromatography (SiO₂, Petroleum ether:Ethyl acetate=10:1 to 1:1) to give the title compound (49-2) (8.10 g, 32.2 mmol, 51.2% yield, 88.6% purity) as a white solid. LCMS (ES⁺): m/z 223.0 [M+H]⁺.

Synthesis of 3-bromo-5-phenyl-1-((2-(trimethylsilyl)ethoxy)methyl)-JH-pyrazole (49-3). To a stirred solution of 3-bromo-5-phenyl-1H-pyrazole (49-2) (4.00 g, 17.9 mmol, 1.00 eq) in THF (40 mL) was added NaH (861 mg, 21.5 mmol, 60.0% purity, 1.20 eq) at 0° C., the mixture was stirred at 0° C. for 0.5 h. SEM-Cl (2.99 g, 17.9 mmol, 3.17 mL, 1.00 eq) was added dropwise at 0° C. The mixture was stirred at 25° C. for 0.5 h. LCMS showed the desired compound was detected. The reaction was quenched by water (30 mL) and extracted with EtOAc (20 mL*3). The combined organic layers were dried over Na₂SO₄, filtered and concentrated under reduced pressure to get a residue. The residue was purified by column chromatography (SiO₂, Petroleum ether:Ethyl acetate=10:1 to 1:1) to give the title compound (49-3) (6.16 g, 17.0 mmol, 95.0% yield, 97.7% purity) as a yellow oil. LCMS (ES⁺): m/z 353.0 [M+H]⁺.

Synthesis of 5-phenyl-3-(4,4,5,5-tetramethyl-1,3,2-dioxaborolan-2-yl)-1-((2-(trimethylsilyl)ethoxy)methyl)-1H-pyrazole (49-4). To a stirred solution of 3-bromo-5-phenyl-1-((2-(trimethylsilyl)ethoxy)methyl)-1H-pyrazole (49-3) (5.00 g, 14.15 mmol, 1.00 eq) and 4,4,5,5-tetramethyl-2-(4,4,5,5-tetramethyl-1,3,2-dioxaborolan-2-yl)-1,3,2-dioxaborolane (10.8 g, 42.5 mmol, 3.00 eq) in toluene (50 mL) were added SPhos Pd G3 (1.10 g, 1.42 mmol, 0.100 eq) and KOAc (2.78 g, 28.3 mmol, 2.00 eq). The mixture was stirred at 80° C. for 12 h under N₂. LCMS showed the desired compound was detected. The reaction mixture was concentrated. The residue was purified by prep-HPLC (column: Phenomenex luna C18 (250*70 mm, 15 um); mobile phase: [water (FA)-ACN]; B %: 50%-80%, 20 min) to give the title compound (49-4) (2.20 g, 5.49 mmol, 38.8% yield) as a yellow solid. LCMS (ES⁺): m/z 401.2 [M+H]⁺.

Synthesis of 2,5-dichloro-4-(5-phenyl-1-((2-(trimethylsilyl)ethoxy)methyl)-JH-pyrazol-3-yl)pyrimidine (49-5). To a stirred solution of 5-phenyl-3-(4,4,5,5-tetramethyl-1,3,2-dioxaborolan-2-yl)-1-((2-(trimethylsilyl)ethoxy)methyl)-1H-pyrazole (49-4) (2.00 g, 5.00 mmol, 1.30 eq) and 2,4,5-trichloropyrimidine (705 mg, 3.84 mmol, 1.00 eq) in dioxane (15 mL) and water (5 mL) were added KOAc (1.13 g, 11.5 mmol, 3.00 eq) and Pd(dppf)Cl₂·CH₂Cl₂ (220 mg, 269 μmol, 0.0700 eq). The mixture was stirred at 60° C. for 12 h under N₂. LCMS showed the desired compound was detected. The reaction mixture was concentrated. The residue was purified by column chromatography (SiO₂, Petroleum ether:Ethyl acetate=10:1 to 1:1) to give the title compound (49-5) (750 mg, 1.37 mmol, 35.8% yield, 77.2% purity) as a colorless oil. LCMS (ES⁺): m/z 421.1 [M+H]⁺.

Synthesis of 1-(5-((5-chloro-4-(5-phenyl-1-((2-(trimethylsilyl)ethoxy)methyl)-1H-pyrazol-3-yl)pyrimidin-2-yl)amino)pyridin-3-yl)pyrrolidin-2-one (49-6). To a stirred solution of 2,5-dichloro-4-(5-phenyl-1-((2-(trimethylsilyl)ethoxy)methyl)-1H-pyrazol-3-yl)pyrimidine (49-5) (250 mg, 593 μmol, 1.00 eq) and 1-(5-amino-3-pyridyl) pyrrolidin-2-one (116 mg, 653 μmol, 1.10 eq) in dioxane (3 mL) were added Cs₂CO₃ (387 mg, 1.19 mmol, 2.00 eq), BINAP (22.2 mg, 35.6 μmol, 0.0600 eq) and Pd₂(dba)₃ (21.7 mg, 23.7 μmol, 0.0400 eq). The mixture was stirred at 100° C. for 2 h under N₂. LCMS showed the desired compound was detected. The reaction mixture was concentrated under reduced pressure to remove solvent. The residue was diluted with H₂O (5 mL) and extracted with EtOAc (20 mL*3). The combined organic layers were washed with water (10 mL*3), dried over Na₂SO₄, filtered and concentrated under reduced pressure to give the title compound (49-6) (500 mg, crude) as a dark brown oil, and it was used into the next step without further purification. LCMS (ES⁺): m/z 584.2 [M+Na]⁺.

Synthesis of 1-(5-((5-chloro-4-(5-phenyl-1H-pyrazol-3-yl)pyrimidin-2-yl)amino)pyridin-3-yl)pyrrolidin-2-one (Compound 49). A solution of 1-(5-((5-chloro-4-(5-phenyl-1-((2-(trimethylsilyl)ethoxy)methyl)-1H-pyrazol-3-yl)pyrimidin-2-yl)amino)pyridin-3-yl)pyrrolidin-2-one (49-6) (500 mg, 890 μmol, 1.00 eq) in HCl/EtOAc (4.00 M, 5 mL) was stirred at 25° C. for 0.5 h. LCMS showed the desired compound was detected. The reaction mixture was concentrated. The residue was purified by prep-HPLC (column: Phenomenex Luna C18 200*40 mm*10 um; mobile phase: [water (FA)-ACN]; B %: 30%-60%, 8 min) to give the title compound (Compound 49) (37.3 mg, 83.5 μmol, 9.39% yield, 96.7% purity) as a white solid. LCMS (ES⁺): m/z 432.1 [M+H]⁺.

### Example 50. Synthesis of 1-(5-((5-chloro-4-(5-cyclopropyl-1H-pyrazol-3-yl)pyrimidin-2-yl)amino)pyridin-3-yl)pyrrolidin-2-one (Compound 50)

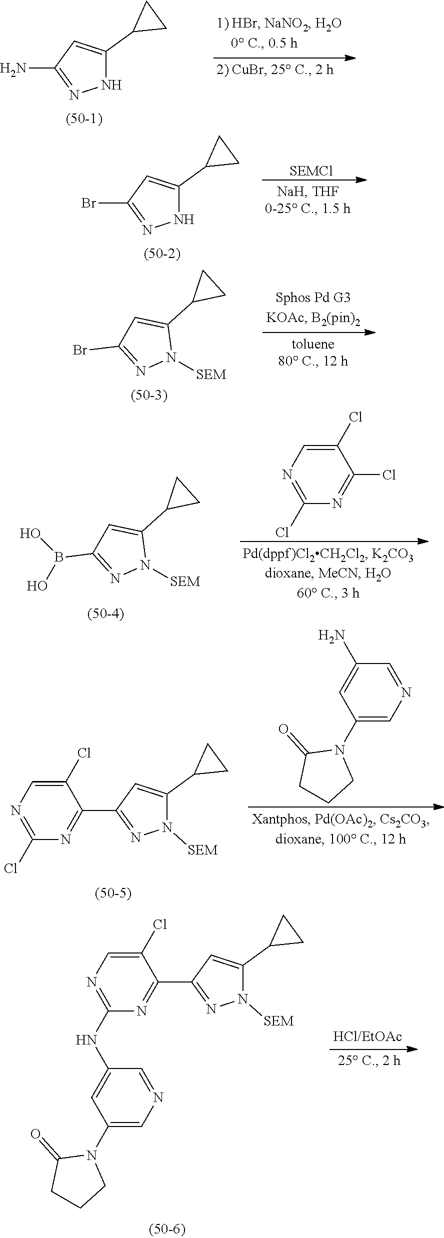

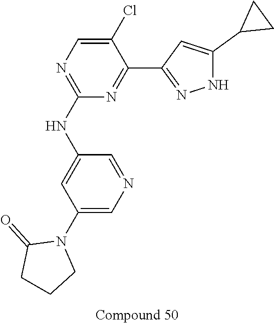 [SMILES: O=C1CCCN1c1cncc(Nc2ncc(Cl)c(-c3cc(C4CC4)[nH]n3)n2)c1]

Synthesis of 3-bromo-5-cyclopropyl-1H-pyrazole (50-2). To a solution of 5-cyclopropyl-1H-pyrazol-3-amine (50-1) (5.00 g, 40.6 mmol, 1.00 eq) and HBr (82.1 g, 406 mmol, 55.1 mL, 40.0% purity, 10.0 eq) in H₂O (30 mL) was added NaNO₂ (3.08 g, 44.7 mmol, 1.10 eq) at 0° C. and then stirred at 0° C. for 0.5 h. CuBr (6.41 g, 44.7 mmol, 1.36 mL, 1.10 eq) was added and the mixture was stirred at 25° C. for 2 h. LCMS showed 5-cyclopropyl-1H-pyrazol-3-amine (50-1) was consumed completely and desired mass was detected. The reaction mixture was quenched by the addition of saturated aqueous KOH (10 mL). The reaction mixture was partitioned between H₂O (100 mL) and EtOAc (100 mL). The organic phase was separated, dried over Na₂SO₄, filtered and concentrated under reduced pressure to give a residue. The residue was purified by flash silica gel chromatography (ISCO®; 40 g SepaFlash® Silica Flash Column, Eluent of 0-100% Ethyl acetate/Petroleum ether gradient @120 mL/min) to give the title compound (50-2) (2.00 g, 10.7 mmol, 26.3% yield) as a white solid. LCMS (ES⁺): m/z 187.0 [M+H]⁺.

Synthesis of 3-bromo-5-cyclopropyl-1-((2-(trimethylsilyl)ethoxy)methyl)-1H-pyrazole (50-3). To a solution of 3-bromo-5-cyclopropyl-1H-pyrazole (50-2) (2.00 g, 10.7 mmol, 1.00 eq) in THF (20 mL) added NaH (513 mg, 12.8 mmol, 60.0% purity, 1.20 eq) portionwise at 0° C. The mixture was stirred at 25° C. for 0.5 h. 2-(chloromethoxy)ethyl-trimethyl-silane (1.78 g, 10.7 mmol, 1.89 mL, 1.00 eq) was added dropwise at 0° C. The mixture was stirred at 25° C. for 1 h. LCMS showed 3-bromo-5-cyclopropyl-1H-pyrazole (50-2) was consumed completely and desired mass was detected. The reaction was quenched by water (30 mL) and extracted with EtOAc (20 mL*3). The combined organic layers were dried over Na₂SO₄, filtered and concentrated under reduced pressure to get a residue. The residue was purified by reversed-phase HPLC (column: Agela C18 330 g; mobile phase: [water (TFA)-ACN]; Gradient Separation: 35-65% 25 min, 65% 15 min) to give the title compound (50-3) (2.00 g, 6.30 mmol, 58.9% yield) as a yellow solid. LCMS (ES⁺): m/z 317.0 [M+H]⁺.

Synthesis of (5-cyclopropyl-1-((2-(trimethylsilyl)ethoxy)methyl)-1H-pyrazol-3-yl)boronic acid (50-4). To a solution of 3-bromo-5-cyclopropyl-1-((2-(trimethylsilyl)ethoxy)methyl)-1H-pyrazole (50-3) (1.90 g, 5.99 mmol, 1.00 eq) and 4,4,5,5-tetramethyl-2-(4,4,5,5-tetramethyl-1,3,2-dioxaborolan-2-yl)-1,3,2-dioxaborolane (4.56 g, 18.0 mmol, 3.00 eq) in toluene (15 mL) were added KOAc (1.18 g, 12.0 mmol, 2.00 eq) and SPhos Pd G3 (467 mg, 599 μmol, 0.100 eq). The mixture was stirred at 80° C. for 12 h under N₂ atmosphere. LCMS showed 3-bromo-5-cyclopropyl-1-((2-(trimethylsilyl)ethoxy)methyl)-1H-pyrazole (50-3) was consumed completely and desired mass was detected. The reaction mixture was concentrated under reduced pressure to get a residue. The residue was purified by prep-HPLC (column: Phenomenex luna C18 (250*70 mm, 15 um); mobile phase: [water (FA)-ACN]; B %: 45%-75%, 20 min) to give the title compound 50-4 (550 mg, 1.95 mmol, 32.6% yield) as a white solid. LCMS (ES⁺): m/z 283.1 [M+H]⁺.

Synthesis of 2,5-dichloro-4-(5-cyclopropyl-1-((2-(trimethylsilyl)ethoxy)methyl)-1H-pyrazol-3-yl)pyrimidine (50-5). To a solution of (5-cyclopropyl-1-((2-(trimethylsilyl)ethoxy)methyl)-1H-pyrazol-3-yl)boronic acid (50-4) (350 mg, 1.24 mmol, 1.00 eq), 2,4,5-trichloropyrimidine (455 mg, 2.48 mmol, 2.00 eq) and Pd(dppf)Cl₂·CH₂Cl₂ (101 mg, 124 μmol, 0.100 eq) in Dioxane (1.5 mL), water (0.5 mL) and MeCN (1.5 mL) was added K₂CO₃ (514 mg, 3.72 mmol, 3.00 eq). The mixture was stirred at 60° C. for 3 h. LCMS showed (5-cyclopropyl-1-((2-(trimethylsilyl)ethoxy)methyl)-1H-pyrazol-3-yl)boronic acid (50-4) was consumed completely and desired mass was detected. The reaction mixture was concentrated under reduced pressure to get a residue. The residue was purified by flash silica gel chromatography (ISCO®; 10 g SepaFlash® Silica Flash Column, Eluent of 0-20% Ethyl acetate/Petroleum ether gradient @100 mL/min) to give the title compound (50-5) (330 mg, 856 μmol, 69.1% yield) as a white solid. LCMS (ES⁺): m/z 385.0 [M+H]⁺.

Synthesis of 1-(5-((5-chloro-4-(5-cyclopropyl-1-((2-(trimethylsilyl)ethoxy)methyl)-1H-pyrazol-3-yl)pyrimidin-2-yl)amino)pyridin-3-yl)pyrrolidin-2-one (50-6). To a solution of 2,5-dichloro-4-(5-cyclopropyl-1-((2-(trimethylsilyl)ethoxy)methyl)-1H-pyrazol-3-yl)pyrimidine (50-5) (310 mg, 804 μmol, 0.800 eq), 1-(5-amino-3-pyridyl) pyrrolidin-2-one (178 mg, 1.01 mmol, 1.00 eq), Cs₂CO₃ (983 mg, 3.02 mmol, 3.00 eq) and Xantphos (116 mg, 201 μmol, 0.200 eq) in Dioxane (3 mL) was added Pd(OAc)₂ (22.6 mg, 101 μmol, 0.100 eq). The mixture was stirred at 100° C. for 12 h under nitrogen. LCMS showed 2, 5-dichloro-4-(5-cyclopropyl-1-((2-(trimethylsilyl)ethoxy)methyl)-1H-pyrazol-3-yl)pyrimidine (50-5) was consumed completely and desired mass was detected. The reaction mixture was concentrated under reduced pressure to get a residue. The reaction was clean according to TLC (Petroleum ether:Ethyl acetate=1:3, R_{f}=0.2). The suspension was filtered and the filter cake was washed with ethyl acetate (30 mL). The combined filtrates were concentrated to dryness to give the title compound (50-6) (160 mg, 259 μmol, 25.7% yield, 85.0% purity) as a white solid. LCMS (ES⁺): m/z 526.2 [M+H]⁺.

Synthesis of 1-(5-((5-chloro-4-(5-cyclopropyl-1H-pyrazol-3-yl)pyrimidin-2-yl)amino)pyridin-3-yl)pyrrolidin-2-one (Compound 50). A solution of 1-(5-((5-chloro-4-(5-cyclopropyl-1-((2-(trimethylsilyl)ethoxy)methyl)-1H-pyrazol-3-yl)pyrimidin-2-yl)amino)pyridin-3-yl)pyrrolidin-2-one (50-6) (150 mg, 285 μmol, 1.00 eq) in HCl/EtOAc (4.00 M, 5 mL) was stirred at 25° C. for 2 h. LCMS showed 1-(5-((5-chloro-4-(5-cyclopropyl-1-((2-(trimethylsilyl)ethoxy)methyl)-1H-pyrazol-3-yl)pyrimidin-2-yl)amino)pyridin-3-yl)pyrrolidin-2-one (50-6) was consumed completely and desired mass was detected. The reaction mixture was concentrated under reduced pressure to get a residue. The reaction mixture was partitioned between H₂O (20 mL) and EtOAc (20 mL). The organic phase was separated, washed with EtOAc (20 mL*3), dried over Na₂SO₄, filtered and concentrated under reduced pressure to give a residue. The residue was purified by prep-HPLC (column: Phenomenex Luna C18 75*30 mm*3 um; mobile phase: [water (FA)-ACN]; B %: 10%-45%, 8 min) to give the title compound (Compound 50) (34.4 mg, 85.6 μmol, 30.0% yield, 98.5% purity) as a white solid. LCMS (ES⁺): m/z 396.1 [M+H]⁺.

### Example 51. Synthesis of N-(5-((4-([1,1′-biphenyl]-3-yl)-5-chloropyrimidin-2-yl)amino)pyridin-3-yl)-10-(2-((3-(2,6-dioxopiperidin-3-yl)-1-methyl-2-oxo-2,3-dihydro-1H-benzo[d]imidazol-5-yl)oxy)acetamido)decanamide (Compound 51)

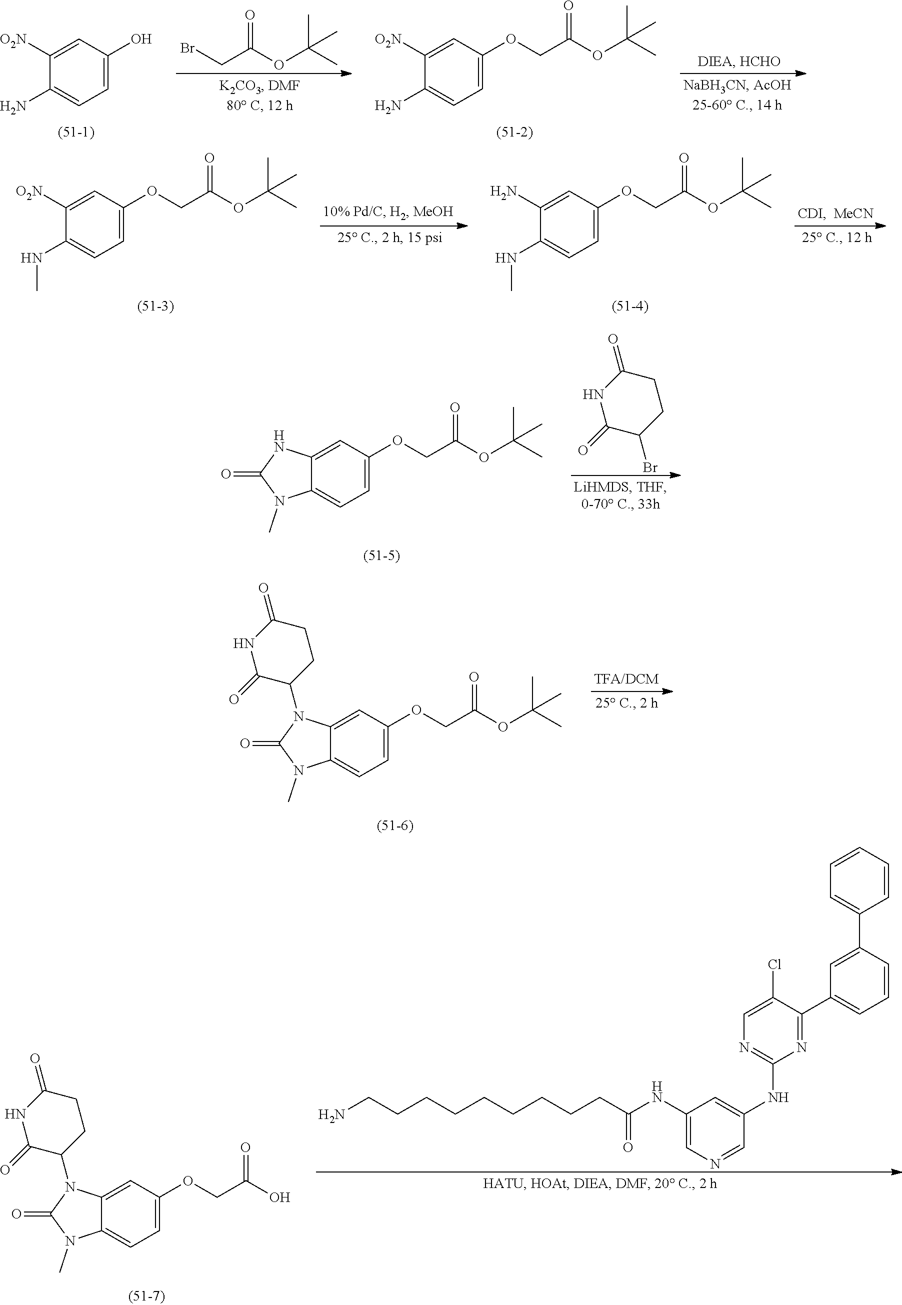

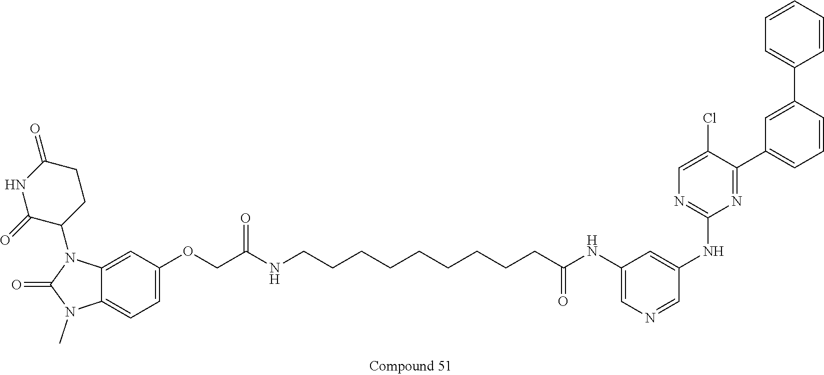 [SMILES: Cn1c(=O)n(C2CCC(=O)NC2=O)c2cc(OCC(=O)NCCCCCCCCCC(=O)Nc3cncc(Nc4ncc(Cl)c(-c5cccc(-c6ccccc6)c5)n4)c3)ccc21]

Synthesis of tert-butyl 2-(4-amino-3-nitrophenoxy)acetate (51-2). To a solution of 4-amino-3-nitrophenol (51-1) (5.00 g, 32.4 mmol, 1.00 eq) in DMF (80 mL) were added tert-butyl 2-bromoacetate (9.49 g, 48.7 mmol, 7.19 mL, 1.50 eq) and K₂CO₃ (8.97 g, 64.9 mmol, 2.00 eq) at 25° C. The mixture was stirred at 80° C. for 12 h. LCMS showed 4-amino-3-nitrophenol (51-1) was consumed completely and the desired mass was detected. Water (100 mL) was added to the reaction, the aqueous phase was extracted with EtOAc (100 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 3:1) to give the title compound (51-2) (6.10 g, 22.7 mmol, 70.1% yield) as a red solid. LCMS (ES⁺): m/z 269.1 [M+H]⁺.

Synthesis of tert-butyl 2-(4-(methylamino)-3-nitrophenoxy)acetate (51-3). To a solution of tert-butyl 2-(4-amino-3-nitrophenoxy)acetate (51-2) (2.00 g, 7.46 mmol, 1.00 eq) and aq. HCHO (3.03 g, 37.3 mmol, 2.78 mL, 37.0% purity, 5.00 eq) in DCM (10 mL) and MeOH (20 mL) was added AcOH (44.8 mg, 746 μmol, 42.6 μL, 0.100 eq) at 25° C. and the mixture was stirred at 40° C. for 0.5 h. Then to the mixture was added NaBH₃CN (2.34 g, 37.3 mmol, 5.00 eq) at 25° C. and the resulting mixture was stirred at 25° C. for 12 h. LCMS showed tert-butyl 2-(4-amino-3-nitrophenoxy)acetate (51-2) remained and the desired mass was detected. To the mixture were added aq. HCHO (3.03 g, 37.3 mmol, 2.78 mL, 37.0% purity, 5.00 eq) and NaBH₃CN (2.34 g, 37.3 mmol, 5.00 eq) at 25° C. The resulting mixture was stirred at 60° C. for 2 h. LCMS showed tert-butyl 2-(4-amino-3-nitrophenoxy)acetate (51-2) was consumed and the desired mass was detected. The mixture was diluted with H₂O (20 mL) and then extracted with EtOAc (10 mL*3). The combined organic layers were dried over Na₂SO₄, filtered and the filtrate was concentrated in vacuo. The mixture was purified by MPLC (SiO₂, Petroleum ether:Ethyl acetate=1:0 to 5:1) to give the title compound (51-3) (2.00 g, 7.08 mmol, 95.0% yield) as a red solid. LCMS (ES⁺): m/z 283.0 [M+H]⁺.

Synthesis of tert-butyl 2-(3-amino-4-(methylamino)phenoxy)acetate (51-4). To a mixture of 10% Pd/C (1.00 g, 50.0% purity, 1.00 eq) in MeOH (20 mL) was added tert-butyl 2-(4-(methylamino)-3-nitrophenoxy)acetate (51-3) (1.00 g, 3.54 mmol, 1.00 eq) at 25° C. The mixture was stirred at 25° C. for 2 h under H₂ (15 psi). LCMS showed tert-butyl 2-(4-(methylamino)-3-nitrophenoxy)acetate (51-3) was consumed and the desired mass was detected. The mixture was filtered, the filtrate was concentrate in vacuo to give the title compound (51-4) (1.00 g, crude) as a black solid. LCMS (ES⁺): m/z 253.3 [M+H]⁺.

Synthesis of tert-butyl 2-((2-oxo-2,3-dihydro-1H-benzo[d]imidazol-5-yl)oxy)acetate (51-5). To a solution of tert-butyl 2-(3-amino-4-(methylamino)phenoxy)acetate (51-4) (1.00 g, 3.96 mmol, 1.00 eq) in MeCN (15 mL) was added CDI (1.29 g, 7.92 mmol, 2.00 eq) at 25° C. The mixture was stirred at 25° C. for 12 h. LCMS showed tert-butyl 2-(3-amino-4-(methylamino)phenoxy)acetate (51-4) was consumed and the desired mass was detected. The mixture was diluted with H₂O (20 mL) and then extracted with EtOAc (15 mL*3). The combined organic layers were dried over Na₂SO₄, filtered and the filtrate was concentrated in vacuo. The mixture was purified by MPLC (SiO₂, Petroleum ether:Ethyl acetate=1:0 to 1:5) to give the title compound (51-5) (500 mg, 1.80 mmol, 45.4% yield) as a white solid. LCMS (ES⁺): m/z 279.1 [M+H]⁺.

Synthesis of tert-butyl 2-((3-(2,6-dioxopiperidin-3-yl)-1-methyl-2-oxo-2,3-dihydro-1H-benzo[d]imidazol-5-yl)oxy)acetate (51-6). To a solution of tert-butyl 2-((2-oxo-2,3-dihydro-1H-benzo[d]imidazol-5-yl)oxy)acetate (51-5) (200 mg, 719 μmol, 1.00 eq) in THF (4 mL) was added LiHMDS (1.00 M, 2.16 mL, 3.00 eq) at 0° C. and the mixture was stirred at 0° C. for 0.5 h. Then to the mixture was added a solution of 3-bromopiperidine-2,6-dione (152 mg, 791 μmol, 1.10 eq) in THF (2 mL) at 0° C. and the mixture was stirred at 70° C. for 12 h. LCMS showed tert-butyl 2-((2-oxo-2,3-dihydro-1H-benzo[d]imidazol-5-yl)oxy)acetate (51-5) remained and the desired mass was detected. Then the resulting mixture was stirred at 70° C. for 8 h. LCMS showed tert-butyl 2-((2-oxo-2,3-dihydro-1H-benzo[d]imidazol-5-yl)oxy)acetate (51-5) remained and the desired mass was detected. To the mixture was added LiHMDS (1.00 M, 1.44 mL, 2.00 eq) at 0° C. and the mixture was stirred at 0° C. for 0.5 h. To the mixture was added 3-bromopiperidine-2,6-dione (152 mg, 791 μmol, 1.10 eq) and THF (2 mL) at 25° C. The resulting mixture was stirred at 70° C. for 12 h. LCMS showed tert-butyl 2-((2-oxo-2,3-dihydro-1H-benzo[d]imidazol-5-yl)oxy)acetate (51-5) remained and the desired mass was detected. The mixture was diluted with H₂O (20 mL) and then extracted with EtOAc (15 mL*3). The combined organic layers were dried over Na₂SO₄, filtered and the filtrate was concentrated in vacuo. The mixture was purified by prep-TLC (SiO₂, PE:EtOAc=1:5) to give the title compound (51-6) (40.0 mg, 103 μmol, 14.3% yield) as a white solid. LCMS (ES⁺): m/z 390.0 [M+H]⁺.

Synthesis of (51-7). The solution of tert-butyl 2-((3-(2,6-dioxopiperidin-3-yl)-1-methyl-2-oxo-2,3-dihydro-1H-benzo[d]imidazol-5-yl)oxy)acetate (51-6) (40.0 mg, 103 μmol, 1.00 eq) in DCM (1.5 mL) and TFA (0.5 mL) was stirred at 25° C. for 2 h. LCMS showed tert-butyl 2-((3-(2,6-dioxopiperidin-3-yl)-1-methyl-2-oxo-2,3-dihydro-1H-benzo[d]imidazol-5-yl)oxy)acetate (51-6) was consumed and the desired mass was detected. The mixture was concentrated in vacuo to give the title compound (51-7) (33.0 mg, crude) as a yellow solid. LCMS (ES⁺): m/z 334.0 [M+H]⁺.

Synthesis of N-(5-((4-([1,1′-biphenyl]-3-yl)-5-chloropyrimidin-2-yl)amino)pyridin-3-yl)-10-(2-((3-(2,6-dioxopiperidin-3-yl)-1-methyl-2-oxo-2,3-dihydro-1H-benzo[d]imidazol-5-yl)oxy)acetamido)decanamide (Compound 51). To a solution of 2-((3-(2,6-dioxopiperidin-3-yl)-1-methyl-2-oxo-2,3-dihydro-1H-benzo[d]imidazol-5-yl)oxy)acetic acid (51-7) (30.0 mg, 67.1 μmol, 1.00 eq) in DMF (0.5 mL) were added HATU (38.3 mg, 101 μmol, 1.50 eq), HOAt (4.56 mg, 33.5 μmol, 4.69 μL, 0.500 eq) and DIEA (26.0 mg, 201 μmol, 35.1 μL, 3.00 eq), then N-(5-((4-([1,1′-biphenyl]-3-yl)-5-chloropyrimidin-2-yl)amino)pyridin-3-yl)-10-aminodecanamide (59.15 mg, 90.01 μmol, 1.00 eq, TFA) was added at 25° C. The mixture was stirred at 25° C. for 2 h. LCMS showed 2-((3-(2,6-dioxopiperidin-3-yl)-1-methyl-2-oxo-2,3-dihydro-1H-benzo[d]imidazol-5-yl)oxy)acetic acid (51-7) was consumed and the desired mass was detected. The mixture was diluted with H₂O (10 mL) and then extracted with EtOAc (5 mL*3). The mixture was concentrated in vacuo. The residue was purified by prep-HPLC (column: Phenomenex luna C18 80*40 mm*3 um; mobile phase: [water (FA)-ACN]; B %: 30%-70%, 8 min) to give the title compound (Compound 51) (5.30 mg, 6.15 μmol, 9.18% yield, 99.7% purity) as a white solid. LCMS (ES⁺): m/z 858.2 [M+H]⁺.

### Example 52. Synthesis of 3-(5-((10-(2-(5-((4-([1,1′-biphenyl]-3-yl)-5-chloropyrimidin-2-yl)amino)pyridin-3-yl)-1-oxo-2,8-diazaspiro[4.5]decan-8-yl)decyl)oxy)-1-oxoisoindolin-2-yl)piperidine-2,6-dione (Compound 52)

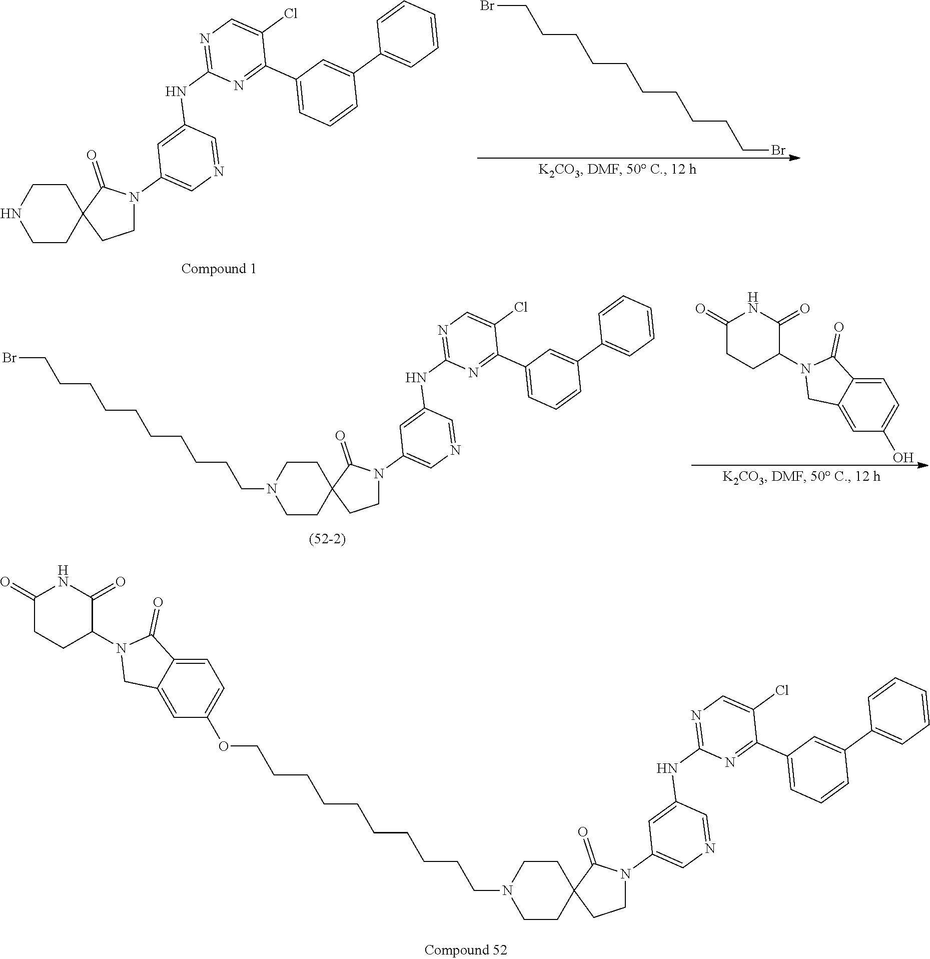 [SMILES: O=C1N(c2cncc(Nc3ncc(Cl)c(-c4cccc(-c5ccccc5)c4)n3)c2)CCC12CCNCC2 ; BrCCCCCCCCCCBr ; O=C1N(c2cncc(Nc3ncc(Cl)c(-c4cccc(-c5ccccc5)c4)n3)c2)CCC12CCN(CCCCCCCCCCBr)CC2 ; O=C1CCC(N2Cc3cc(O)ccc3C2=O)C(=O)N1 ; O=C1CCC(N2Cc3cc(OCCCCCCCCCCN4CCC5(CC4)CCN(c4cncc(Nc6ncc(Cl)c(-c7cccc(-c8ccccc8)c7)n6)c4)C5=O)ccc3C2=O)C(=O)N1]

Synthesis of 2-(5-((4-([1,1′-biphenyl]-3-yl)-5-chloropyrimidin-2-yl)amino)pyridin-3-yl)-8-(10-bromodecyl)-2,8-diazaspiro[4.5]decan-1-one (52-2). To a solution of 2-(5-((4-([1,1′-biphenyl]-3-yl)-5-chloropyrimidin-2-yl)amino)pyridin-3-yl)-2,8-diazaspiro[4.5]decan-1-one (Compound 1) (100 mg, 196 μmol, 1.00 eq) and 1,10-dibromodecane (64.6 mg, 215 μmol, 1.10 eq) in DMF (3 mL) was added K₂CO₃ (81.1 mg, 587 μmol, 3.00 eq). The mixture was stirred at 50° C. for 12 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) remained and the desired mass was detected. The reaction mixture was partitioned between EtOAc (30 mL) and H₂O (10 mL). The organic phase was separated, washed with brine (10 mL), dried over Na₂SO₄, filtered and concentrated under reduced pressure to give a residue. The residue was purified by prep-TLC (SiO₂, Ethyl acetate:MeOH=10:1) to give the title compound (52-2) (60.0 mg, 82.2 μmol, 42.0% yield) as a brown solid. LCMS (ES⁺): m/z 729.2 [M+H]⁺.

Synthesis of 3-(5-((10-(2-(5-((4-([1,1′-biphenyl]-3-yl)-5-chloropyrimidin-2-yl)amino)pyridin-3-yl)-1-oxo-2,8-diazaspiro[4.5]decan-8-yl)decyl)oxy)-1-oxoisoindolin-2-yl)piperidine-2,6-dione (Compound 52). To a solution of 3-(5-hydroxy-1-oxo-isoindolin-2-yl)piperidine-2,6-dione (21.4 mg, 82.2 μmol, 1.20 eq) and 2-(5-((4-([1,1′-biphenyl]-3-yl)-5-chloropyrimidin-2-yl)amino)pyridin-3-yl)-8-(10-bromodecyl)-2,8-diazaspiro[4.5]decan-1-one (52-2) (50.0 mg, 68.5 μmol, 1.00 eq) in DMF (2 mL) was added K₂CO₃ (28.4 mg, 205 μmol, 3.00 eq). The mixture was stirred at 50° C. for 12 h. LCMS showed 2-(5-((4-([1,1′-biphenyl]-3-yl)-5-chloropyrimidin-2-yl)amino)pyridin-3-yl)-8-(10-bromodecyl)-2,8-diazaspiro[4.5]decan-1-one (52-2) remained and desired mass was detected. The reaction mixture was concentrated under reduced pressure to remove solvent. The residue was purified by prep-HPLC (column: Phenomenex luna C18 80*40 mm*3 um; mobile phase: [water (FA)-ACN]; B %: 35%-40%, 8 min) to give the title compound (Compound 52) (2.40 mg, 2.64 μmol, 3.85% yield) as a white solid. LCMS (ES⁺): m/z 909.51 [M+H]⁺.

### Example 53. Synthesis of 3-(5-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)nonyl)oxy)-1-oxoisoindolin-2-yl)piperidine-2,6-dione (Compound 53)

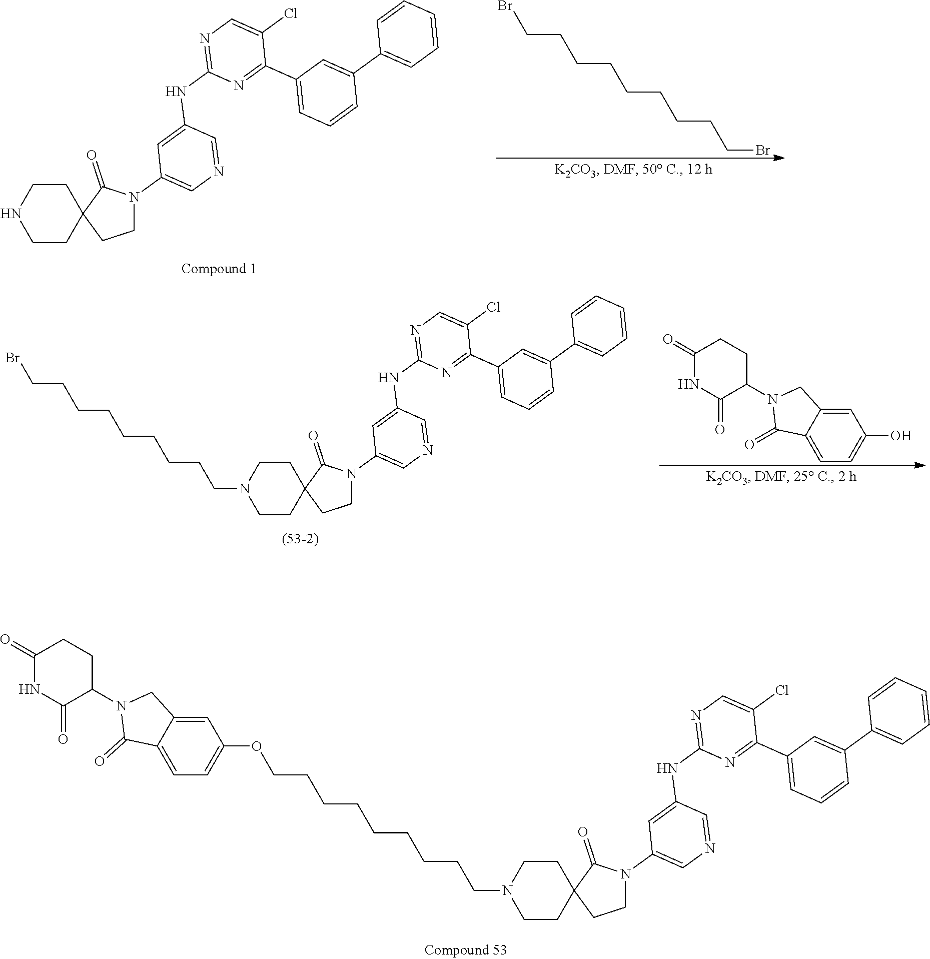

Synthesis of 2-(5-((4-([1,1′-biphenyl]-3-yl)-5-chloropyrimidin-2-yl)amino)pyridin-3-yl)-8-(9-bromononyl)-2,8-diazaspiro[4.5]decan-1-one (53-2). To a solution of 2-(5-((4-([1,1′-biphenyl]-3-yl)-5-chloropyrimidin-2-yl)amino)pyridin-3-yl)-2,8-diazaspiro[4.5]decan-1-one (Compound 1) (100 mg, 196 μmol, 1.00 eq) and 1,9-dibromononane (61.6 mg, 215 μmol, 1.10 eq) in DMF (3 mL) was added K₂CO₃ (81.1 mg, 587 μmol, 3.00 eq). The mixture was stirred at 50° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) remained and desired mass was detected. The reaction mixture was partitioned between EtOAc (30 mL) and H₂O (10 mL). The organic phase was separated, washed with brine (10 mL), dried over Na₂SO₄, filtered and concentrated under reduced pressure to give a residue. The residue was purified by column chromatography by prep-TLC (SiO₂, Ethyl acetate:MeOH=10:1) to give the title compound (53-2) (60.0 mg, 83.8 μmol, 42.8% yield) as a brown solid. LCMS (ES⁺): m/z 715.1 [M+H]⁺.

Synthesis of 3-(5-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)nonyl)oxy)-1-oxoisoindolin-2-yl)piperidine-2,6-dione (Compound 53). To a solution of 2-(5-((4-([1,1′-biphenyl]-3-yl)-5-chloropyrimidin-2-yl)amino)pyridin-3-yl)-8-(9-bromononyl)-2,8-diazaspiro[4.5]decan-1-one (53-2) (50.0 mg, 69.8 μmol, 1.00 eq) and 3-(5-hydroxy-1-oxo-isoindolin-2-yl)piperidine-2,6-dione (21.8 mg, 83.8 μmol, 1.20 eq) in DMF (2 mL) was added K₂CO₃ (29.0 mg, 209 μmol, 3.00 eq). The mixture was stirred at 25° C. for 2 h. LCMS showed 2-(5-((4-([1,1′-biphenyl]-3-yl)-5-chloropyrimidin-2-yl)amino)pyridin-3-yl)-8-(9-bromononyl)-2,8-diazaspiro[4.5]decan-1-one (53-2) remained and desired mass was detected. The reaction mixture was concentrated under reduced pressure. The residue was purified by prep-HPLC (column: Phenomenex luna C18 80*40 mm*3 um; mobile phase: [water (FA)-ACN]; B %: 35%-40%, 8 min) to give the title compound (Compound 53) (4.10 mg, 4.58 μmol, 6.56% yield, 100% purity) as a brown solid. LCMS (ES⁺): m/z 895.3 [M+H]⁺.

### Example 54. Synthesis of 3-(5-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)heptyl)oxy)-1-oxoisoindolin-2-yl)piperidine-2,6-dione (Compound 54)

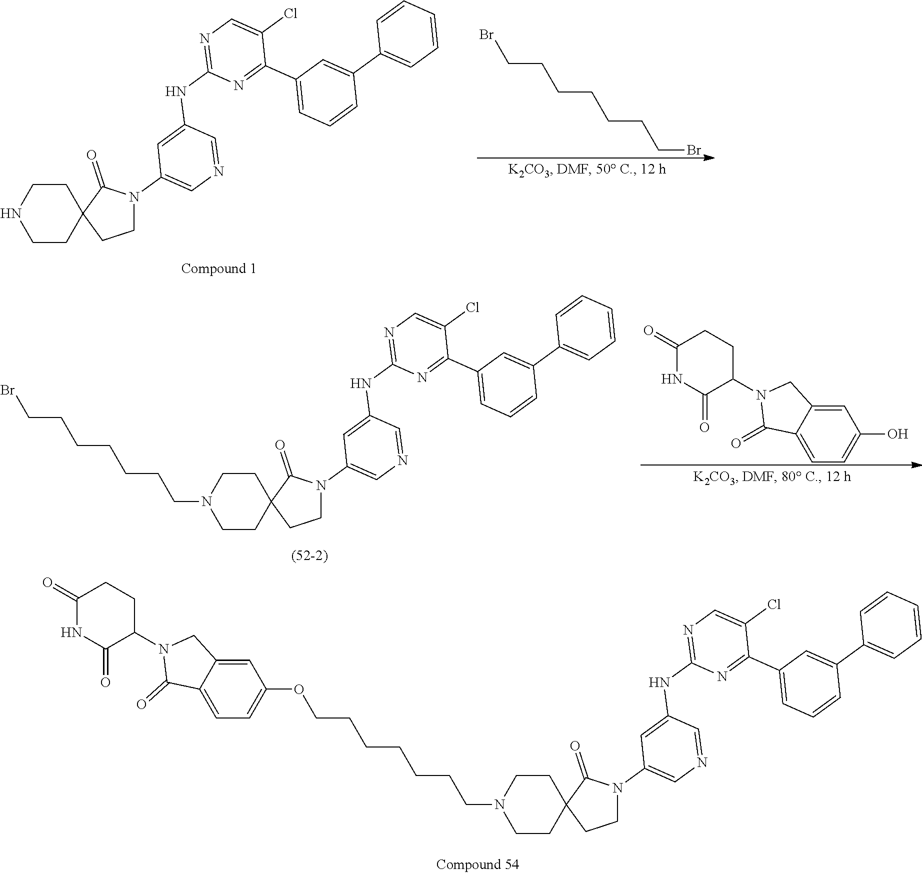

Synthesis of 2-(5-((4-([1,1′-biphenyl]-3-yl)-5-chloropyrimidin-2-yl)amino)pyridin-3-yl)-8-(7-bromoheptyl)-2,8-diazaspiro[4.5]decan-1-one (54-2). A mixture of 1,7-dibromoheptane (151 mg, 587 μmol, 36.2 μL, 1.50 eq), 2-(5-((4-([1,1′-biphenyl]-3-yl)-5-chloropyrimidin-2-yl)amino)pyridin-3-yl)-2,8-diazaspiro[4.5]decan-1-one (Compound 1) (200 mg, 391 μmol, 1.00 eq) and K₂CO₃ (162 mg, 1.17 mmol, 3.00 eq) in DMF (2 mL) was stirred at 50° C. for 12 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely. Several new peaks were shown on LCMS and 65% of desired compound was detected. The reaction mixture was diluted with H₂O (3 mL) and extracted with EtOAc (2 mL*3). The combined organic layers were dried over Na₂SO₄, filtered and concentrated under reduced pressure to give a residue. The residue was purified by prep-TLC (SiO₂, Ethyl acetate:MeOH=10:1) to give the title compound (54-2) (80.0 mg, 102 μmol, 26.1% yield, 88.0% purity) as a yellow solid. LCMS (ES⁺): m/z 687.2 [M+H]⁺.

Synthesis of 3-(5-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)heptyl)oxy)-1-oxoisoindolin-2-yl)piperidine-2,6-dione (Compound 54). A mixture of 3-(5-hydroxy-1-oxo-isoindolin-2-yl)piperidine-2,6-dione (22.7 mg, 87.2 μmol, 1.00 eq), 2-(5-((4-([1,1′-biphenyl]-3-yl)-5-chloropyrimidin-2-yl)amino)pyridin-3-yl)-8-(7-bromoheptyl)-2,8-diazaspiro[4.5]decan-1-one (54-2) (60.0 mg, 87.2 μmol, 1.00 eq) and K₂CO₃ (36.2 mg, 262 μmol, 3.00 eq) in DMF (1 mL) was stirred at 80° C. for 12 h. LCMS showed 2-(5-((4-([1,1′-biphenyl]-3-yl)-5-chloropyrimidin-2-yl)amino)pyridin-3-yl)-8-(7-bromoheptyl)-2,8-diazaspiro[4.5]decan-1-one (54-2) was consumed completely. Several new peaks were shown on LCMS and desired compound was detected. The reaction mixture was concentrated to give a residue. The residue was purified by prep-HPLC (column: Phenomenex luna C18 80*40 mm*3 um; mobile phase: [water (FA)-ACN]; B %: 35%-40%, 8 min) to give the title compound (Compound 54) (18.0 mg, 20.5 μmol, 23.5% yield, 98.8% purity) as a white solid. LCMS (ES⁺): m/z 867.4 [M+H]⁺.

### Example 55. Synthesis of N-(5-((4-([1,1′-biphenyl]-3-yl)-5-chloropyrimidin-2-yl)amino)pyridin-3-yl)-10-(2-(4-((2,6-dioxopiperidin-3-yl)amino)phenoxy)acetamido)decanamide (Compound 55)

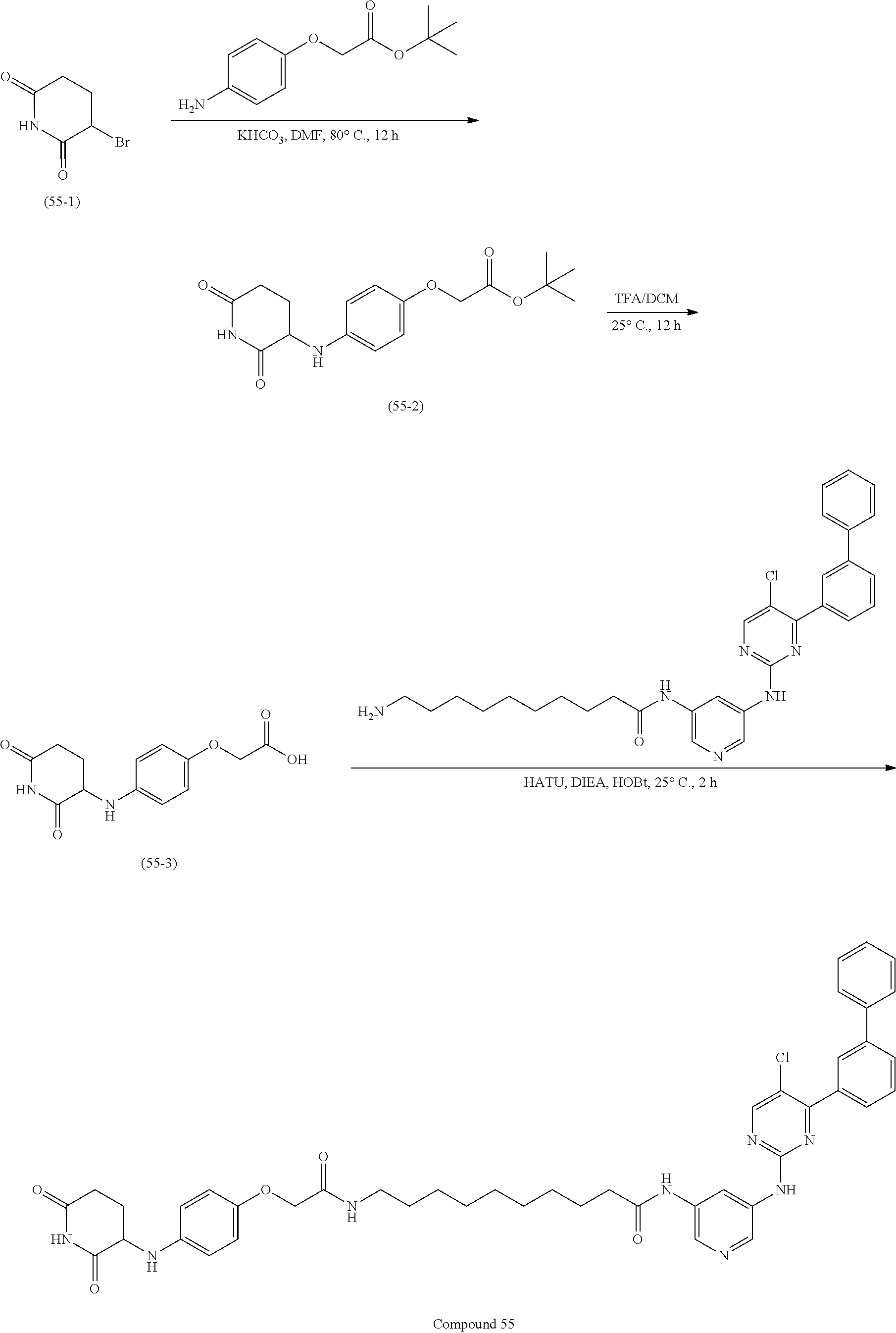 [SMILES: O=C1CCC(Br)C(=O)N1 ; CC(C)(C)OC(=O)COc1ccc(N)cc1 ; CC(C)(C)OC(=O)COc1ccc(NC2CCC(=O)NC2=O)cc1 ; O=C(O)COc1ccc(NC2CCC(=O)NC2=O)cc1 ; NCCCCCCCCCC(=O)Nc1cncc(Nc2ncc(Cl)c(-c3cccc(-c4ccccc4)c3)n2)c1 ; O=C(COc1ccc(NC2CCC(=O)NC2=O)cc1)NCCCCCCCCCC(=O)Nc1cncc(Nc2ncc(Cl)c(-c3cccc(-c4ccccc4)c3)n2)c1]

Synthesis of tert-butyl 2-(4-((2,6-dioxopiperidin-3-yl)amino)phenoxy)acetate (55-2). To a solution of 3-bromopiperidine-2,6-dione (55-1) (2.80 g, 12.5 mmol, 1.00 eq) in DMF (30 mL) were added KHCO₃ (3.77 g, 37.6 mmol, 3.00 eq) and tert-butyl 2-(4-aminophenoxy)acetate (3.61 g, 18.8 mmol, 1.50 eq) at 25° C. The mixture was stirred at 80° C. for 12 h under N₂ atmosphere. LCMS showed 3-bromopiperidine-2,6-dione (55-1) was consumed partly and the desired mass was detected. Water (20 mL) was added to the mixture at 25° C. Then the reaction mixture was extracted with EtOAc (6 mL*3). The combined organic layers were dried over anhydrous sodium sulfate, filtered and the filtrate was concentrated under reduced pressure to give a residue. The residue was purified by column chromatography (SiO₂, Petroleum ether:Ethyl acetate=1:0 to 1:1) to give the title compound (55-2) (1.40 g, crude) as a green solid. LCMS (ES⁺): m/z 335.2 [M+H]⁺.

Synthesis of 2-(4-((2,6-dioxopiperidin-3-yl)amino)phenoxy)acetic acid (55-3). A solution of tert-butyl 2-(4-((2,6-dioxopiperidin-3-yl)amino)phenoxy)acetate (55-2) (500 mg, 1.50 mmol, 1.00 eq) in TFA (2 mL) and DCM (10 mL) was stirred at 25° C. for 12 h. LCMS showed tert-butyl 2-(4-((2,6-dioxopiperidin-3-yl)amino)phenoxy)acetate (55-2) was consumed completely and the desired mass was detected. The reaction mixture was concentrated under reduced pressure to give the title compound (55-3) (560 mg, crude) as a white solid. LCMS (ES⁺): m/z 279.2 [M+H]⁺.

Synthesis of N-(5-((4-([1,1′-biphenyl]-3-yl)-5-chloropyrimidin-2-yl)amino)pyridin-3-yl)-10-(2-(4-((2,6-dioxopiperidin-3-yl)amino)phenoxy)acetamido)decanamide (Compound 55). To a solution of 2-(4-((2,6-dioxopiperidin-3-yl)amino)phenoxy)acetic acid (55-3) (80.5 mg, 289 μmol, 1.00 eq) in DMF (5 mL) were added N-(5-((4-([1,1′-biphenyl]-3-yl)-5-chloropyrimidin-2-yl)amino)pyridin-3-yl)-10-aminodecanamide (190 mg, 289 μmol, 1.00 eq, TFA) (as prepared in Example 66 or 85), HATU (165 mg, 434 μmol, 1.50 eq), DIEA (116 mg, 896 μmol, 156 μL, 3.10 eq) and HOBt (58.6 mg, 434 μmol, 1.50 eq) at 25° C. The mixture was stirred at 25° C. for 2 h. LCMS showed 2-(4-((2,6-dioxopiperidin-3-yl)amino)phenoxy)acetic acid (55-3) was consumed completely and the desired mass was detected. Water (5 mL) was added to reaction, the aqueous phase was extracted with EtOAc (3 mL*3), the combined organic layers were dried over Na₂SO₄, filtered and the filtrate was concentrated under reduced pressure. The residue was purified by prep-HPLC (column: Phenomenex Luna C18 200*40 mm*10 um; mobile phase: [water (FA)-ACN]; B %: 40%-80%, 8 min) to give the title compound (Compound 55) (42.1 mg, 51.0 μmol, 17.6% yield, 97.3% purity) as a brown solid. LCMS (ES⁺): m/z 803.3 [M+H]⁺.

### Example 56. Synthesis of 3-(5-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)octyl)oxy)-1-oxoisoindolin-2-yl)piperidine-2,6-dione (Compound 56)

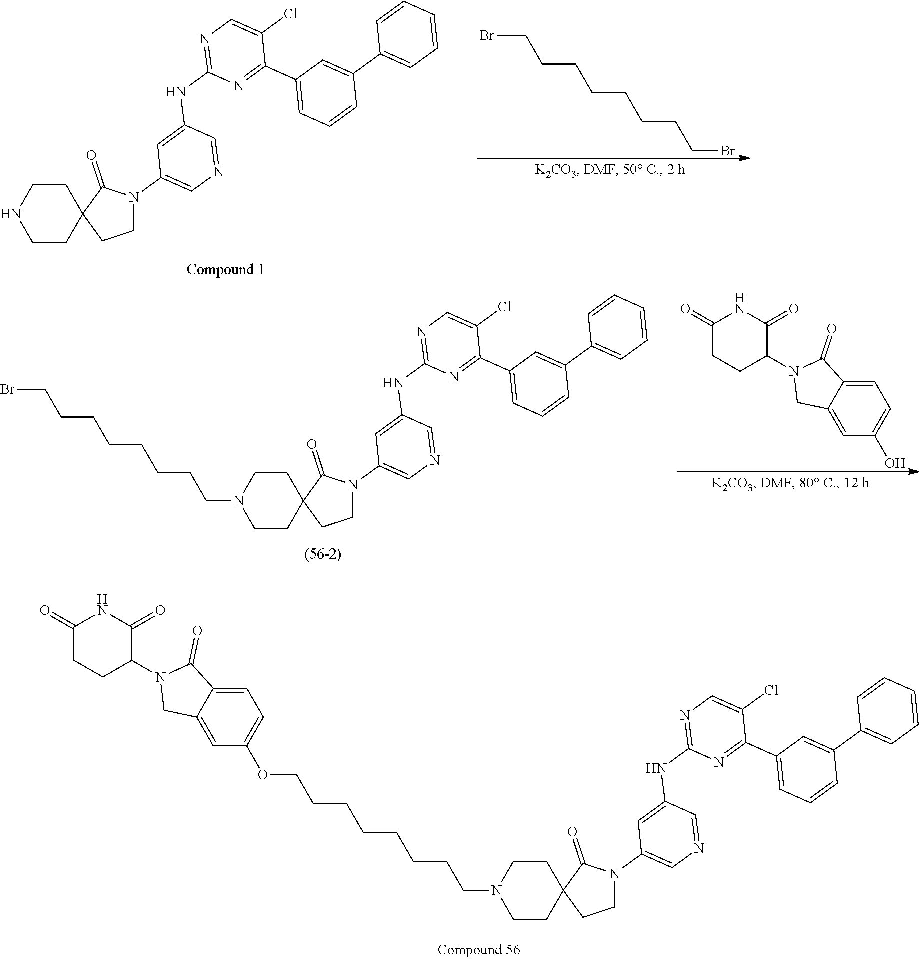

Synthesis of 2-(5-((4-([1,1′-biphenyl]-3-yl)-5-chloropyrimidin-2-yl)amino)pyridin-3-yl)-8-(8-bromooctyl)-2,8-diazaspiro[4.5]decan-1-one (56-2). A mixture of 1,8-dibromooctane (160 mg, 587 μmol, 109 μL, 1.50 eq), 2-(5-((4-([1,1′-biphenyl]-3-yl)-5-chloropyrimidin-2-yl)amino)pyridin-3-yl)-2,8-diazaspiro[4.5]decan-1-one (Compound 1) (200 mg, 391 μmol, 1.00 eq) and K₂CO₃ (162 mg, 1.17 mmol, 3.00 eq) in DMF (2 mL) was stirred at 50° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely. Several new peaks were shown on LCMS and 75% of desired compound was detected. The reaction mixture was diluted with H₂O (3 mL) and extracted with EtOAc (2 mL*3). The combined organic layers were dried over Na₂SO₄, filtered and concentrated under reduced pressure to give a residue. The residue was purified by prep-TLC (SiO₂, Ethyl acetate:MeOH=10:1) to give the title compound (56-2) (80.0 mg, 114 μmol, 29.1% yield) as a yellow solid. LCMS (ES⁺): m/z 701.2 [M+H]⁺.

Synthesis of 3-(5-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)octyl)oxy)-1-oxoisoindolin-2-yl)piperidine-2,6-dione (Compound 56). A mixture of 3-(5-hydroxy-1-oxo-isoindolin-2-yl)piperidine-2,6-dione (22.2 mg, 85.5 μmol, 1.00 eq), 2-(5-((4-([1,1′-biphenyl]-3-yl)-5-chloropyrimidin-2-yl)amino)pyridin-3-yl)-8-(8-bromooctyl)-2,8-diazaspiro[4.5]decan-1-one (56-2) (60.0 mg, 85.5 μmol, 1.00 eq) and K₂CO₃ (35.4 mg, 256 μmol, 3.00 eq) in DMF (1 mL) was stirred at 80° C. for 12 h. LCMS showed 2-(5-((4-([1,1′-biphenyl]-3-yl)-5-chloropyrimidin-2-yl)amino)pyridin-3-yl)-8-(8-bromooctyl)-2,8-diazaspiro[4.5]decan-1-one (56-2) was consumed completely. Several new peaks were shown on LCMS and desired compound was detected. The reaction mixture was concentrated to give a residue. The residue was purified by prep-HPLC (column: Phenomenex luna C18 80*40 mm*3 um; mobile phase: [water (FA)-ACN]; B %: 35%-40%, 8 min) to give the title compound (Compound 56) (10.9 mg, 12.2 μmol, 14.2% yield, 98.3% purity) as a white solid. LCMS (ES⁺): m/z 881.5 [M+H]⁺.

### Example 57. Synthesis of 3-((4-(4-(2-(5-((5-chloro-4-(piperidin-1-yl)pyrimidin-2-yl)amino)pyridin-3-yl)-1-oxo-2,8-diazaspiro[4.5]decan-8-yl)piperidin-1-yl)phenyl)amino)piperidine-2,6-dione (Compound 91)

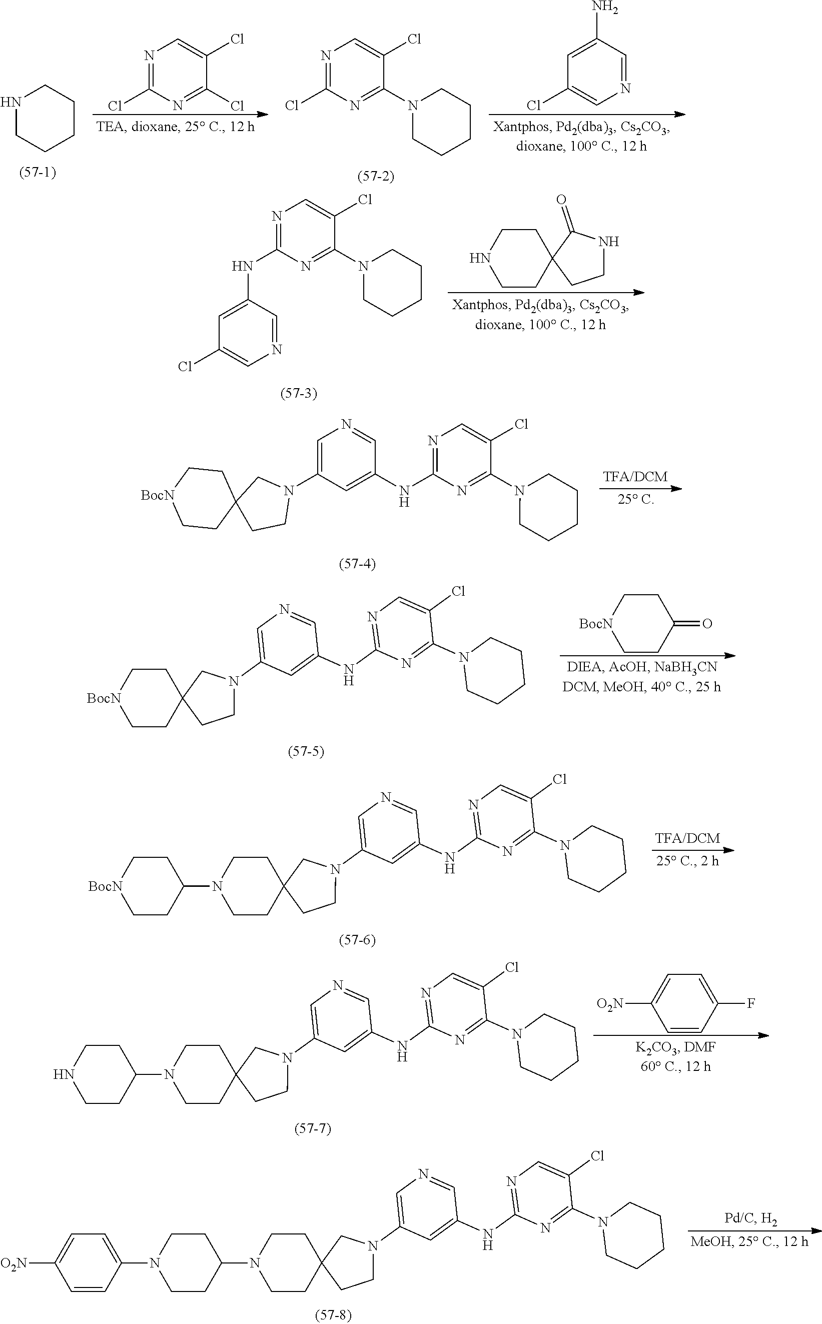

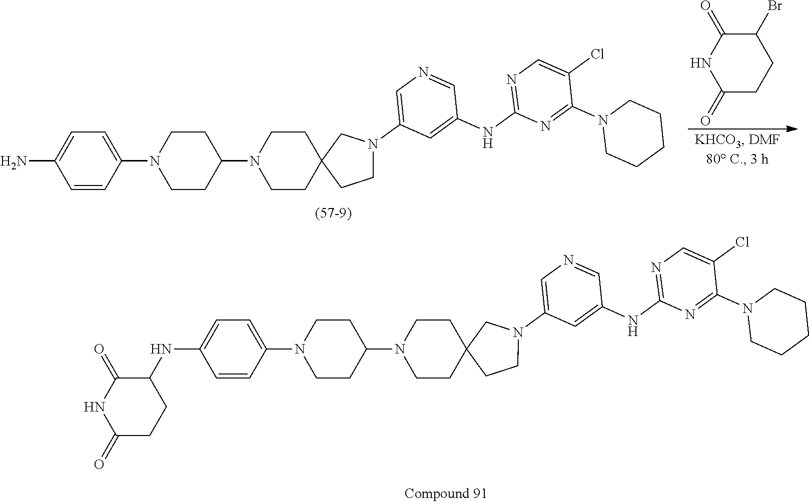

Synthesis of 2,5-dichloro-4-(piperidin-1-yl)pyrimidine (57-2). To a solution of piperidine (57-1) (306 mg, 3.60 mmol, 355 μL, 1.20 eq) in dioxane (6 mL) were added TEA (334 mg, 3.30 mmol, 459 μL, 1.10 eq) and 2,4,5-trichloropyrimidine (550 mg, 3.00 mmol, 1.00 eq) at 25° C. under N₂ atmosphere. The mixture was stirred at 25° C. for 12 h. LCMS showed piperidine 57-1 was consumed completely and the desired mass was detected. The reaction mixture was concentrated under reduced pressure to remove solvent. The residue was purified by prep-TLC (SiO₂, Petroleum ether:Ethyl acetate=10:1) to give the title compound (57-2) (300 mg, 1.29 mmol, 43.1% yield) as a white solid. LCMS (ES⁺): m/z 232.0 [M+H]⁺.

Synthesis of N-(5-bromopyridin-3-yl)-5-chloro-4-(piperidin-1-yl)pyrimidin-2-amine (57-3). To a solution of 2,5-dichloro-4-(1-piperidyl)pyrimidine (57-2) (20.0 g, 86.17 mmol, 1.00 eq) and 5-bromopyridin-3-amine (16.40 g, 94.78 mmol, 1.10 eq) in dioxane (800 mL) were added Cs₂CO₃ (56.15 g, 172.33 mmol, 2.00 eq), BINAP (5.37 g, 8.62 mmol, 0.100 eq) and Pd₂(dba)₃ (3.95 g, 4.31 mmol, 0.05 eq) at 20° C. under N₂ atmosphere. The mixture was stirred at 100° C. for 12 h under N₂ atmosphere. LCMS showed the desired mass. The mixture was concentrated in vacuo. The mixture was diluted with H₂O (1000 mL) and then extracted with EtOAc (150 mL*3). The combined organic layers were dried over Na₂SO₄, filtered and the filtrate was concentrated in vacuo. The residue was purified by column chromatography (SiO₂, Petroleum ether/Ethyl acetate=1/0 to 3/1) to give the title compound (57-3) (12.51 g, crude) as a brown solid. LCMS (ES⁺): m/z 368.1 [M+H]⁺.

Synthesis of tert-butyl 2-(5-((5-chloro-4-(piperidin-1-yl)pyrimidin-2-yl)amino)pyridin-3-yl)-1-oxo-2,8-diazaspiro[4.5]decane-8-carboxylate (57-4). To a solution of N-(5-bromo-3-pyridyl)-5-chloro-4-(1-piperidyl)pyrimidin-2-amine (57-3) (7.00 g, 18.99 mmol, 1.00 eq) and tert-butyl 1-oxo-2,8-diazaspiro[4.5]decane-8-carboxylate (3.86 g, 15.19 mmol, 0.800 eq) in dioxane (120 mL) were added Cs₂CO₃ (7.42 g, 22.79 mmol, 1.20 eq) Xantphos (988.80 mg, 1.71 mmol, 0.09 eq) and Pd₂(dba)₃ (521.62 mg, 569.63 μmol, 0.03 eq) at 25° C. under N₂ atmosphere. The mixture was stirred at 100° C. for 12 hr. LCMS showed the desired mass. The mixture was concentrated in vacuo. The mixture was diluted with H₂O (300 mL) and then extracted with EtOAc (60 mL*3). The combined organic layers were dried over Na₂SO₄, filtered and the filtrate was concentrated in vacuo. The residue was purified by column chromatography (SiO₂, Petroleum ether/Ethyl acetate=1/0 to 1/3) to give the title compound (57-4) (9.51 g, 17.54 mmol, 92.4% yield) as a white solid. LCMS (ES⁺): m/z 486.2 [M+H−56]⁺.

Synthesis of tert-butyl 4-(2-(5-((5-chloro-4-(piperidin-1-yl)pyrimidin-2-yl)amino)pyridin-3-yl)-1-oxo-2,8-diazaspiro[4.5]decan-8-yl)piperidine-1-carboxylate (57-6). To a solution of 2-(5-((5-chloro-4-(piperidin-1-yl)pyrimidin-2-yl)amino)pyridin-3-yl)-2,8-diazaspiro[4.5]decan-1-one (57-5) (3.00 g, 5.40 mmol, 1.00 eq, TFA) in DCM (20 mL) and MeOH (20 mL) were added DIEA (3.49 g, 27.0 mmol, 4.70 mL, 5.00 eq) to adjust pH to 8-9 at 25° C. The mixture was stirred at 25° C. for 0.5 h. Then tert-butyl 4-oxopiperidine-1-carboxylate (7.53 g, 37.8 mmol, 7.00 eq) and AcOH (32.4 mg, 540 μmol, 30.9 μL, 0.100 eq) were added to the mixture at 25° C., ensure pH to 5-6. The mixture was stirred at 25° C. for 0.5 h. Then NaBH₃CN (2.38 g, 37.8 mmol, 7.00 eq) was added to the mixture at 25° C. The mixture was stirred at 40° C. for 12 h. LCMS showed 2-(5-((5-chloro-4-(piperidin-1-yl)pyrimidin-2-yl)amino)pyridin-3-yl)-2,8-diazaspiro[4.5]decan-1-one (57-5) was remaining, but no desired mass was detected. Then tert-butyl 4-oxopiperidine-1-carboxylate (7.53 g) and CH₃COOH (1 mL) were added to the mixture at 40° C. The mixture was stirred at 40° C. for 12 h. LCMS showed ˜11% of 2-(5-((5-chloro-4-(piperidin-1-yl)pyrimidin-2-yl)amino)pyridin-3-yl)-2,8-diazaspiro[4.5]decan-1-one (57-5) was remaining, and ˜23% of the desired mass was detected. The reaction mixture was concentrated under reduced pressure to remove solvent. Water (20 mL) was added to the mixture at 25° C. Then the reaction mixture was extracted with DCM (30*3 mL). The organic layers were dried over anhydrous sodium sulfate, filtered and the filtrate was concentrated under reduced pressure to give a residue. The residue was purified by column chromatography (SiO₂, Petroleum ether:Ethyl acetate=10:1 to 1:1) to give the title compound (57-6) (5.00 g, 4.16 mmol, 77.0% yield, 52.0% purity) as a yellow oil. LCMS (ES⁺): m/z 625.4 [M+H]⁺.

Synthesis of 2-(5-((5-chloro-4-(piperidin-1-yl)pyrimidin-2-yl)amino)pyridin-3-yl)-8-(piperidin-4-yl)-2,8-diazaspiro[4.5]decan-1-one (57-7). To a solution of tert-butyl 4-(2-(5-((5-chloro-4-(piperidin-1-yl)pyrimidin-2-yl)amino)pyridin-3-yl)-1-oxo-2,8-diazaspiro[4.5]decan-8-yl)piperidine-1-carboxylate (57-6) (5.00 g, 8.00 mmol, 1.00 eq) in DCM (30 mL) was added TFA (30.0 g, 263 mmol, 10.0 mL, 32.9 eq) at 25° C. The mixture was stirred at 25° C. for 2 h. LCMS showed tert-butyl 4-(2-(5-((5-chloro-4-(piperidin-1-yl)pyrimidin-2-yl)amino)pyridin-3-yl)-1-oxo-2,8-diazaspiro[4.5]decan-8-yl)piperidine-1-carboxylate (57-6) was consumed completely and the desired mass was detected. The reaction mixture was concentrated under reduced pressure to remove solvent to give the title compound (57-7) (5.10 g, crude, TFA) as a brown oil. LCMS (ES⁺): m/z 525.3 [M+H]⁺.

Synthesis of 2-(5-((5-chloro-4-(piperidin-1-yl)pyrimidin-2-yl)amino)pyridin-3-yl)-8-(1-(4-nitrophenyl)piperidin-4-yl)-2,8-diazaspiro[4.5]decan-1-one (57-8). To a solution of 2-(5-((5-chloro-4-(piperidin-1-yl)pyrimidin-2-yl)amino)pyridin-3-yl)-8-(piperidin-4-yl)-2,8-diazaspiro[4.5]decan-1-one (57-7) (5.10 g, 7.98 mmol, 1.00 eq, TFA) in DMF (10 mL) were added 1-fluoro-4-nitro-benzene (1.13 g, 7.98 mmol, 847 μL, 1.00 eq) and K₂CO₃ (2.21 g, 16.0 mmol, 2.00 eq) at 25° C. The mixture was stirred at 60° C. for 12 h. LCMS showed 2-(5-((5-chloro-4-(piperidin-1-yl)pyrimidin-2-yl)amino)pyridin-3-yl)-8-(piperidin-4-yl)-2,8-diazaspiro[4.5]decan-1-one (57-7) was consumed completely and the desired mass was detected. Water (10 mL) was added to the mixture at 25° C. Then the reaction mixture was extracted with EtOAc (15*3 mL). The organic layers were dried over anhydrous sodium sulfate, filtered and the filtrate was concentrated under reduced pressure to give a residue. The residue was purified by column chromatography (SiO₂, Petroleum ether:Ethyl acetate=1:1 to 1:10) to give the title compound (57-8) (400 mg, 619 μmol, 7.76% yield) as a yellow solid. LCMS (ES⁺): m/z 646.4 [M+H]⁺.

Synthesis of 8-(1-(4-aminophenyl)piperidin-4-yl)-2-(5-((5-chloro-4-(piperidin-1-yl)pyrimidin-2-yl)amino)pyridin-3-yl)-2,8-diazaspiro[4.5]decan-1-one (57-9). 10% Pd/C (500 mg, 50% purity) was added to MeOH (5 mL) at 25° C. under N₂ atmosphere, then 2-(5-((5-chloro-4-(piperidin-1-yl)pyrimidin-2-yl)amino)pyridin-3-yl)-8-(1-(4-nitrophenyl)piperidin-4-yl)-2,8-diazaspiro[4.5]decan-1-one (57-8) (350 mg, 542 μmol, 1.00 eq) was added to the mixture at 25° C., the suspension was degassed and purged with H₂ for three times. The mixture was stirred at 25° C. for 12 h under H₂ (15 psi) atmosphere. LCMS showed 2-(5-((5-chloro-4-(piperidin-1-yl)pyrimidin-2-yl)amino)pyridin-3-yl)-8-(1-(4-nitrophenyl)piperidin-4-yl)-2,8-diazaspiro[4.5]decan-1-one (57-8) was consumed completely and the desired mass was detected. The reaction mixture was filtered and concentrated under reduced pressure to give a residue. The residue was purified by prep-HPLC (column: Phenomenex C18 80*40 mm*3 um; mobile phase: [water (NH₄HCO₃)-ACN]; B %: 40%-70%, 8 min) to give the title compound (57-9) (30.0 mg, 48.7 μmol, 8.99% yield) as a white solid. LCMS (ES⁺): m/z 616.4 [M+H]⁺.

Synthesis of 3-((4-(4-(2-(5-((5-chloro-4-(piperidin-1-yl)pyrimidin-2-yl)amino)pyridin-3-yl)-1-oxo-2,8-diazaspiro[4.5]decan-8-yl)piperidin-1-yl)phenyl)amino)piperidine-2,6-dione (Compound 91). To a solution of (3R)-3-bromopiperidine-2,6-dione (18.7 mg, 97.4 μmol, 2.00 eq) in DMF (2 mL) were added 8-(1-(4-aminophenyl)piperidin-4-yl)-2-(5-((5-chloro-4-(piperidin-1-yl)pyrimidin-2-yl)amino)pyridin-3-yl)-2,8-diazaspiro[4.5]decan-1-one (57-9) (30.0 mg, 48.7 μmol, 1.00 eq) and KHCO₃ (14.6 mg, 146 μmol, 3.00 eq) at 25° C. The mixture was stirred at 80° C. for 1 h. LC-MS showed 8-(1-(4-aminophenyl)piperidin-4-yl)-2-(5-((5-chloro-4-(piperidin-1-yl)pyrimidin-2-yl)amino)pyridin-3-yl)-2,8-diazaspiro[4.5]decan-1-one (57-9) was remaining, and the desired mass was detected. Then the mixture was stirred at 80° C. for 2 h. LCMS showed 8-(1-(4-aminophenyl)piperidin-4-yl)-2-(5-((5-chloro-4-(piperidin-1-yl)pyrimidin-2-yl)amino)pyridin-3-yl)-2,8-diazaspiro[4.5]decan-1-one (57-9) was consumed completely, and the desired mass was detected. The reaction mixture was filtered to give the filtrate. The filtrate was purified by prep-HPLC (column: Waters Xbridge Prep OBD C18 150*40 mm*10 um; mobile phase: [water (NH₄HCO₃)-ACN]; B %: 35%-65%, 8 min) to give the title compound (Compound 91) (8.50 mg, 11.4 μmol, 23.3% yield, 97.2% purity) as a white solid. LCMS (ES⁺): m/z 727.3 [M+H]⁺.

### Example 58. Synthesis of N-(5-((4-([1,1′-biphenyl]-3-yl)-5-chloropyrimidin-2-yl)amino)pyridin-3-yl)-7-(4-(3-((2-(2,6-dioxopiperidin-3-yl)-1,3-dioxoisoindolin-4-yl)oxy)propyl)piperazin-1-yl)heptanamide (Compound 58)

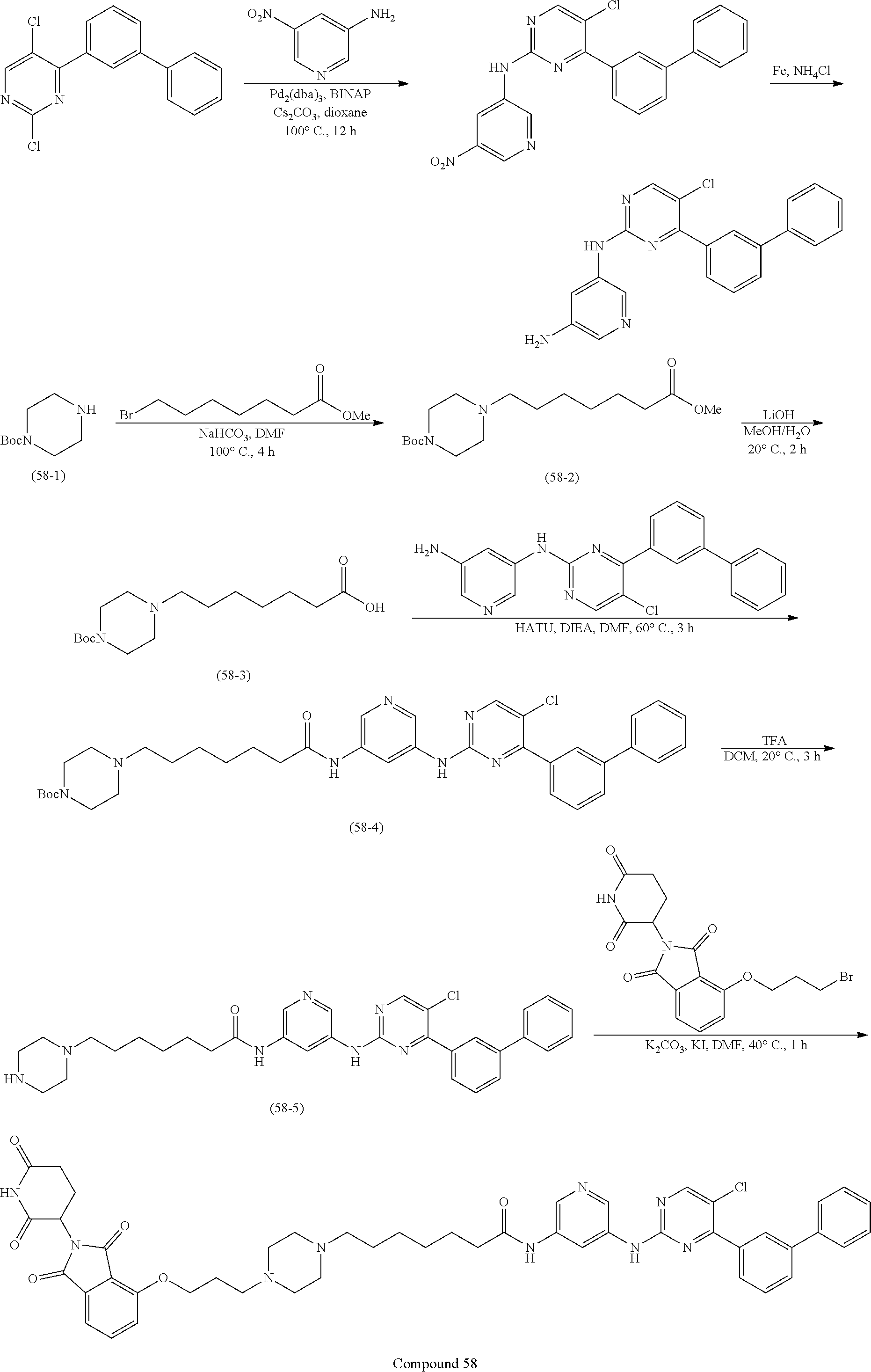

Synthesis of 4-([1,1′-biphenyl]-3-yl)-5-chloro-N-(5-nitropyridin-3-yl)pyrimidin-2-amine. To a solution of 5-nitropyridin-3-amine (20 g, 143.77 mmol, 1.1 eq) and 2,5-dichloro-4-(3-phenylphenyl)pyrimidine (39.36 g, 130.70 mmol, 1 eq) in dioxane (600 mL) was added BINAP (4.88 g, 7.84 mmol, 0.06 eq), Pd₂(dba)₃ (4.79 g, 5.23 mmol, 0.04 eq) and Cs₂CO₃ (85.17 g, 261.40 mmol, 2 eq). The mixture was stirred at 100° C. for 12 h. LC-MS showed the desired product mass. The reaction mixture was concentrated. The residue was dissolved in ethyl acetate (200 mL), and 200 g of silica gel was added. The resulting mixture was concentrated to give a dry flowing solid, and then it was loaded to Biotage using two 330 g flash silica gel columns (SiO₂, Petroleum ether:Ethyl acetate=1:0 to 0:1) to give the title compound (28 g, 69.34 mmol, 53.05% yield) as a pink solid. LCMS (ES⁺): m/z 404.3 [M+H]⁺.

Synthesis of N³-(4-([1,1′-biphenyl]-3-yl)-5-chloropyrimidin-2-yl)pyridine-3,5-diamine. To a solution of 5-chloro-N-(5-nitro-3-pyridyl)-4-(3-phenylphenyl)pyrimidin-2-amine (28 g, 69.34 mmol, 1 eq) in EtOH (350 mL) and Water (70 mL) was added Fe (19.36 g, 346.69 mmol, 5 eq) and NH₄Cl (37.09 g, 693.38 mmol, 10 eq). The mixture was stirred at 80° C. for 2 h. LC-MS showed the desired product mass. The reaction mixture was filtered and concentrated under reduced pressure to give the title compound (17.3 g, 46.28 mmol, 66.74% yield) as a red solid. It was used into the next step without further purification. LCMS (ES⁺): m/z 374.1 [M+H]⁺.

Synthesis of tert-butyl 4-(7-methoxy-7-oxoheptyl)piperazine-1-carboxylate (58-2). A mixture of tert-butyl piperazine-1-carboxylate (58-1) (3.71 g, 19.9 mmol, 1.00 eq), methyl 7-bromoheptanoate (5.00 g, 19.9 mmol, 1.00 eq), NaHCO₃ (5.02 g, 59.7 mmol, 2.32 mL, 3.00 eq) in DMF (40 mL) was stirred at 100° C. for 4 h. LCMS showed tert-butyl piperazine-1-carboxylate (58-1) was consumed and the desired MS was detected. The reaction mixture was diluted with H₂O (40 mL) and extracted with ethyl acetate (20 mL*3). The combined organic layers were dried over Na₂SO₄, filtered and concentrated under reduced pressure to give the title compound (58-2) (7.00 g, crude) as a yellow oil. LCMS (ES⁺): m/z 329.2 [M+H]⁺.

Synthesis of 7-(4-(tert-butoxycarbonyl)piperazin-1-yl)heptanoic acid (58-3). To a solution of tert-butyl 4-(7-methoxy-7-oxoheptyl)piperazine-1-carboxylate (58-2) (3.50 g, 9.82 mmol, 1.00 eq) in MeOH (25 mL) and H₂O (5 mL) was added LiOH (705 mg, 29.5 mmol, 3.00 eq). The mixture was stirred at 20° C. for 2 h. LCMS showed tert-butyl 4-(7-methoxy-7-oxoheptyl)piperazine-1-carboxylate (58-2) was consumed and the desired MS was detected. The reaction mixture was concentrated in vacuo to remove MeOH, the aqueous phase was diluted with H₂O (40 mL), acidified with 1 M of HCl to pH=5, and extracted with ethyl acetate (20 mL*3). The combined organic layers were dried over Na₂SO₄, filtered and concentrated under reduced pressure to give the title compound (58-3) (1.25 g, crude) as a white solid. LCMS (ES⁺): m/z 315.2 [M+H]⁺.

Synthesis of tert-butyl 4-(7-((5-((4-([1,1′-biphenyl]-3-yl)-5-chloropyrimidin-2-yl)amino)pyridin-3-yl)amino)-7-oxoheptyl)piperazine-1-carboxylate (58-4). To a solution of 7-(4-(tert-butoxycarbonyl)piperazin-1-yl)heptanoic acid (58-3) (500 mg, 1.52 mmol, 1.00 eq) and N³-(4-([1,1′-biphenyl]-3-yl)-5-chloropyrimidin-2-yl)pyridine-3,5-diamine (512 mg, 1.37 mmol, 0.900 eq) (prepared as described in Example 58) in DMF (10 mL) were added DIEA (590 mg, 4.57 mmol, 795 μL, 3.00 eq) and HATU (695 mg, 1.83 mmol, 1.20 eq). The mixture was stirred at 60° C. for 3 h. LCMS showed 7-(4-(tert-butoxycarbonyl)piperazin-1-yl)heptanoic acid (58-3) was consumed and desired MS was detected. The reaction mixture was diluted with H₂O (10 mL) and extracted with ethyl acetate (10 mL*3). The combined organic layers were dried over Na₂SO₄, filtered and concentrated under reduced pressure to give the title compound (58-4) (1.04 g, crude) as a brown oil. LCMS (ES⁺): m/z 670.3 [M+H]⁺.

Synthesis of N-(5-((4-([1,1′-biphenyl]-3-yl)-5-chloropyrimidin-2-yl)amino)pyridin-3-yl)-7-(piperazin-1-yl)heptanamide (58-5). To a solution of tert-butyl 4-(7-((5-((4-([1,1′-biphenyl]-3-yl)-5-chloropyrimidin-2-yl)amino)pyridin-3-yl)amino)-7-oxoheptyl)piperazine-1-carboxylate (58-4) (1.09 g, 1.66 mmol, 1.00 eq) in DCM (10 mL) was added TFA (3.08 g, 27.0 mmol, 2 mL, 16.3 eq). The mixture was stirred at 20° C. for 3 h. LCMS showed tert-butyl 4-(7-((5-((4-([1,1′-biphenyl]-3-yl)-5-chloropyrimidin-2-yl)amino)pyridin-3-yl)amino)-7-oxoheptyl)piperazine-1-carboxylate (58-4) was consumed and the desired MS was detected. The reaction mixture was concentrated under reduced pressure to give a residue. The residue was purified by prep-HPLC (column: Phenomenex Luna C18 75*30 mm*3 um; mobile phase: [water (FA)-ACN]; B %: 15%-45%, 8 min) to give the title compound (58-5) (600 mg, 1.08 mmol, 65.0% yield) as a white solid. LCMS (ES⁺): m/z 570.2 [M+H]⁺.

Synthesis of N-(5-((4-([1,1′-biphenyl]-3-yl)-5-chloropyrimidin-2-yl)amino)pyridin-3-yl)-7-(4-(3-((2-(2,6-dioxopiperidin-3-yl)-1,3-dioxoisoindolin-4-yl)oxy)propyl)piperazin-1-yl)heptanamide (Compound 58). To a solution of N-(5-((4-([1,1′-biphenyl]-3-yl)-5-chloropyrimidin-2-yl)amino)pyridin-3-yl)-7-(piperazin-1-yl)heptanamide (58-5) (80.0 mg, 140 μmol, 1.00 eq) and 4-(3-bromopropoxy)-2-(2,6-dioxo-3-piperidyl)isoindoline-1,3-dione (55.5 mg, 140 μmol, 1.00 eq) in DMF (2 mL) were added K₂CO₃ (58.2 mg, 421 μmol, 3.00 eq) and KI (11.7 mg, 70.2 μmol, 0.500 eq). The mixture was stirred at 40° C. for 1 h. LCMS showed N-(5-((4-([1,1′-biphenyl]-3-yl)-5-chloropyrimidin-2-yl)amino)pyridin-3-yl)-7-(piperazin-1-yl)heptanamide (58-5) was consumed and desired MS was detected. The reaction mixture was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 50%-90%, 8 min) to give the title compound (Compound 58) (7.00 mg, 7.78 μmol, 5.54% yield, 98.3% purity) as a white solid. LCMS (ES⁺): m/z 884.4 [M+H]⁺.

### Example 59. Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1,3-dioxoisoindolin-5-yl)oxy)acetamido)decanamide (Compound 59)

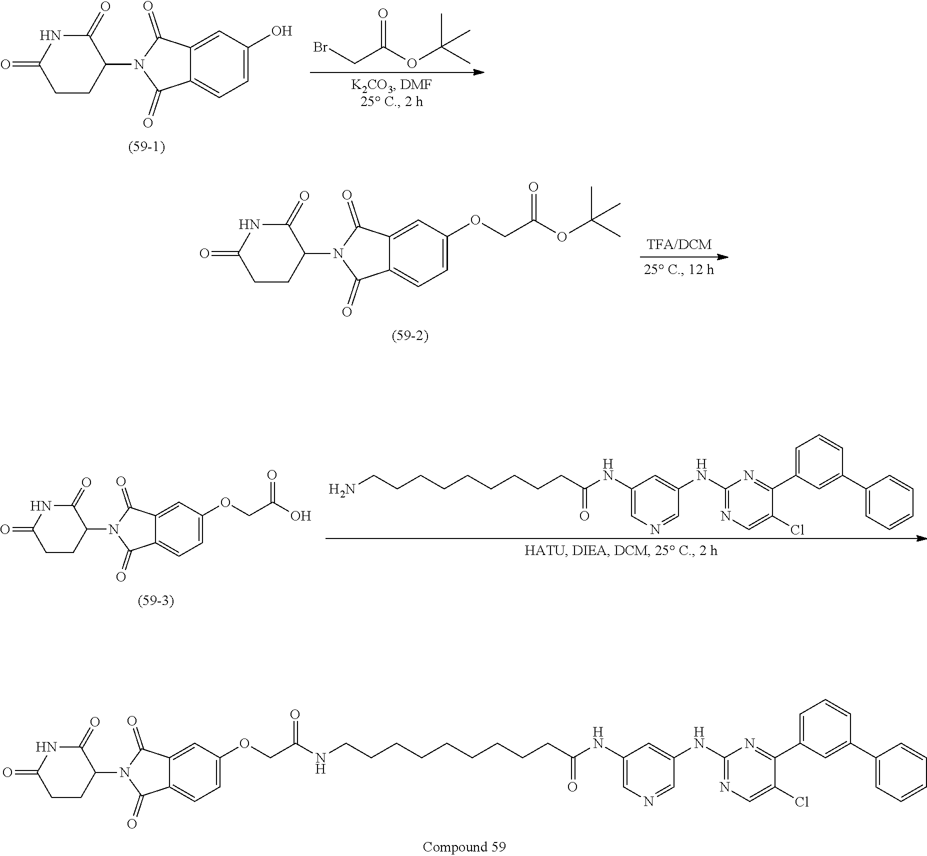

Synthesis of tert-butyl 2-((2-(2,6-dioxopiperidin-3-yl)-1,3-dioxoisoindolin-5-yl)oxy)acetate (59-2). To a solution of 2-(2,6-dioxopiperidin-3-yl)-5-hydroxyisoindoline-1,3-dione (59-1) (300 mg, 1.09 mmol, 1.00 eq) in DMF (5 mL) were added K₂CO₃ (454 mg, 3.28 mmol, 3.00 eq) and tert-butyl 2-bromoacetate (213 mg, 1.09 mmol, 162 μL, 1.00 eq) at 25° C. The mixture was stirred at 25° C. for 2 h. LCMS showed 2-(2,6-dioxopiperidin-3-yl)-5-hydroxyisoindoline-1,3-dione (59-1) was consumed completely and the desired mass was detected. Water (5 mL) was added to the mixture at 25° C. Then the mixture was extracted with EtOAc (4 mL*3). The organic layers were dried over anhydrous sodium sulfate, filtered and the filtrate was concentrated under reduced pressure to give residue. The residue was purified by prep-TLC (SiO₂, Ethyl acetate:Petroleum ether=3:1) to give the title compound (59-2) (380 mg, 978 μmol, 89.4% yield) as a white solid. LCMS (ES⁺): m/z 389.1 [M+H]⁺.

Synthesis of 2-((2-(2,6-dioxopiperidin-3-yl)-1,3-dioxoisoindolin-5-yl)oxy)acetic acid (59-3). To a solution of tert-butyl 2-((2-(2,6-dioxopiperidin-3-yl)-1,3-dioxoisoindolin-5-yl)oxy)acetate (59-2) (120 mg, 309 μmol, 1.00 eq) in DCM (2.5 mL) was added TFA (770 mg, 6.75 mmol, 0.500 mL, 21.9 eq) at 25° C. The mixture was stirred at 25° C. for 12 h. LCMS showed tert-butyl 2-((2-(2,6-dioxopiperidin-3-yl)-1,3-dioxoisoindolin-5-yl)oxy)acetate (59-2) was consumed completely and the desired mass was detected. The reaction mixture was concentrated under reduced pressure to remove solvent to give the title compound (59-3) (102 mg, crude) as a white solid. LCMS (ES⁺): m/z 333.1 [M+H]⁺.

Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1,3-dioxoisoindolin-5-yl)oxy)acetamido)decanamide (Compound 59). To a solution of 2-((2-(2,6-dioxopiperidin-3-yl)-1,3-dioxoisoindolin-5-yl)oxy)acetic acid (59-3) (101 mg, 304 μmol, 1.00 eq) in DCM (5 mL) were added HATU (127 mg, 335 μmol, 1.10 eq), 10-amino-N-[5-[[5-chloro-4-(3-phenylphenyl)pyrimidin-2-yl]amino]-3-pyridyl]decanamide (200 mg, 304 μmol, 1.00 eq, TFA) (as prepared in Example 66 or 85) and DIEA (118 mg, 913 μmol, 159 μL, 3.00 eq) at 25° C. The mixture was stirred at 25° C. for 2 h. LCMS showed 2-((2-(2,6-dioxopiperidin-3-yl)-1,3-dioxoisoindolin-5-yl)oxy)acetic acid (59-3) was consumed completely and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH₄HCO₃)-ACN]; B %: 55%-85%, 8 min) to give the title compound (Compound 59) (16.6 mg, 18.7 μmol, 6.14% yield, 96.5% purity) as a white solid LCMS (ES⁺): m/z 857.2 [M+H]⁺.

### Example 60. Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1-oxoisoindolin-5-yl)oxy)acetamido)decanamide (Compound 60)

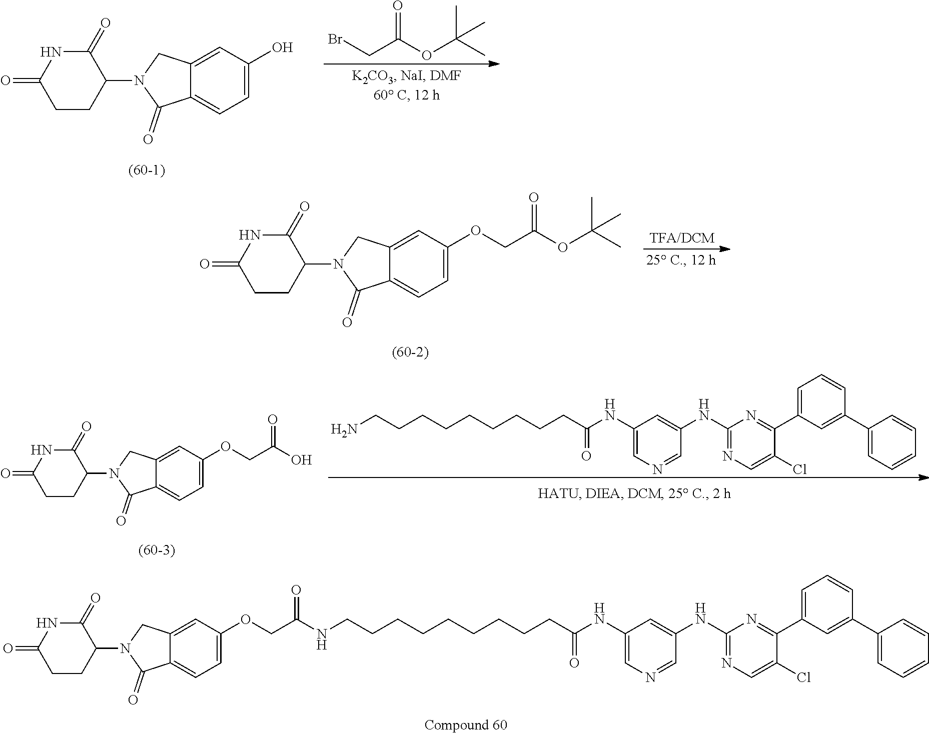

Synthesis of tert-butyl 2-((2-(2,6-dioxopiperidin-3-yl)-1-oxoisoindolin-5-yl)oxy)acetate (60-2). To a solution of 3-(5-hydroxy-1-oxoisoindolin-2-yl)piperidine-2,6-dione (60-1) (1.00 g, 3.84 mmol, 1.00 eq) and tert-butyl 2-bromoacetate (675 mg, 3.46 mmol, 511 μL, 0.900 eq) in DMF (20 mL) were added K₂CO₃ (1.06 g, 7.69 mmol, 2.00 eq) and NaI (115 mg, 769 μmol, 0.200 eq) at 25° C. The resulting mixture was stirred at 60° C. for 12 h. LCMS showed 3-(5-hydroxy-1-oxoisoindolin-2-yl)piperidine-2,6-dione (60-1) was consumed and the desired mass was detected. H₂O (20 mL) was added to the solution, the mixture was extracted with EtOAc (20 mL*3), then the combined organic layers were washed with brine (20 mL*3), dried over Na₂SO₄, then concentrated in vacuo to give the crude product. The mixture was purified by MPLC (SiO₂, Petroleum ether:Ethyl acetate=1:0 to 1:1) to give the title compound (60-2) (850 mg, 2.27 mmol, 59.1% yield) as a white solid. LCMS (ES⁺): m/z 375.0 [M+H]⁺.

Synthesis of 2-((2-(2,6-dioxopiperidin-3-yl)-1-oxoisoindolin-5-yl)oxy)acetic acid (60-3). To a solution of tert-butyl 2-((2-(2,6-dioxopiperidin-3-yl)-1-oxoisoindolin-5-yl)oxy)acetate (60-2) (850 mg, 2.27 mmol, 1.00 eq) in DCM (8 mL) was added TFA (1.6 mL) at 25° C. The resulting mixture was stirred at 25° C. for 12 h. LCMS showed tert-butyl 2-((2-(2,6-dioxopiperidin-3-yl)-1-oxoisoindolin-5-yl)oxy)acetate (60-2) was consumed and the desired mass was detected. The mixture was concentrated in vacuo to give the title compound (60-3) (510 mg, crude) as a yellow solid. LCMS (ES⁺): m/z 319.0 [M+H]⁺.

Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1-oxoisoindolin-5-yl)oxy)acetamido)decanamide (Compound 60). To a solution of 2-((2-(2,6-dioxopiperidin-3-yl)-1-oxoisoindolin-5-yl)oxy)acetic acid (60-3) (92.0 mg, 289 μmol, 1.00 eq) and N-(5-((4-([1,1′-biphenyl]-3-yl)-5-chloropyrimidin-2-yl)amino)pyridin-3-yl)-10-aminodecanamide (190 mg, 289 μmol, 1.00 eq, TFA) (as prepared in Example 66 or 85) in DCM (3 mL) were added HATU (165 mg, 434 μmol, 1.50 eq) and DIEA (112 mg, 867 μmol, 151 μL, 3.00 eq) at 25° C. The resulting mixture was stirred at 25° C. for 2 h. LCMS showed 2-((2-(2,6-dioxopiperidin-3-yl)-1-oxoisoindolin-5-yl)oxy)acetic acid (60-3) was consumed and the desired mass was detected. The mixture was concentrated in vacuo. The residue was purified by prep-HPLC (column: Phenomenex Luna C18 200*40 mm*10 um; mobile phase: [water (FA)-ACN]; B %: 30%-65%, 8 min) to give the title compound (Compound 60) (20.1 mg, 23.1 μmol, 7.98% yield, 96.8% purity) as a white solid. LCMS (ES⁺): m/z 843.2 [M+H]⁺.

### Example 61. Synthesis of 3-(5-((10-(2-(5-((4-([1,1′-biphenyl]-3-yl)-5-chloropyrimidin-2-yl)amino)pyridin-3-yl)-1-oxo-2,8-diazaspiro[4.5]decan-8-yl)-10-oxodecyl)oxy)-1-oxoisoindolin-2-yl)piperidine-2,6-dione (Compound 61)

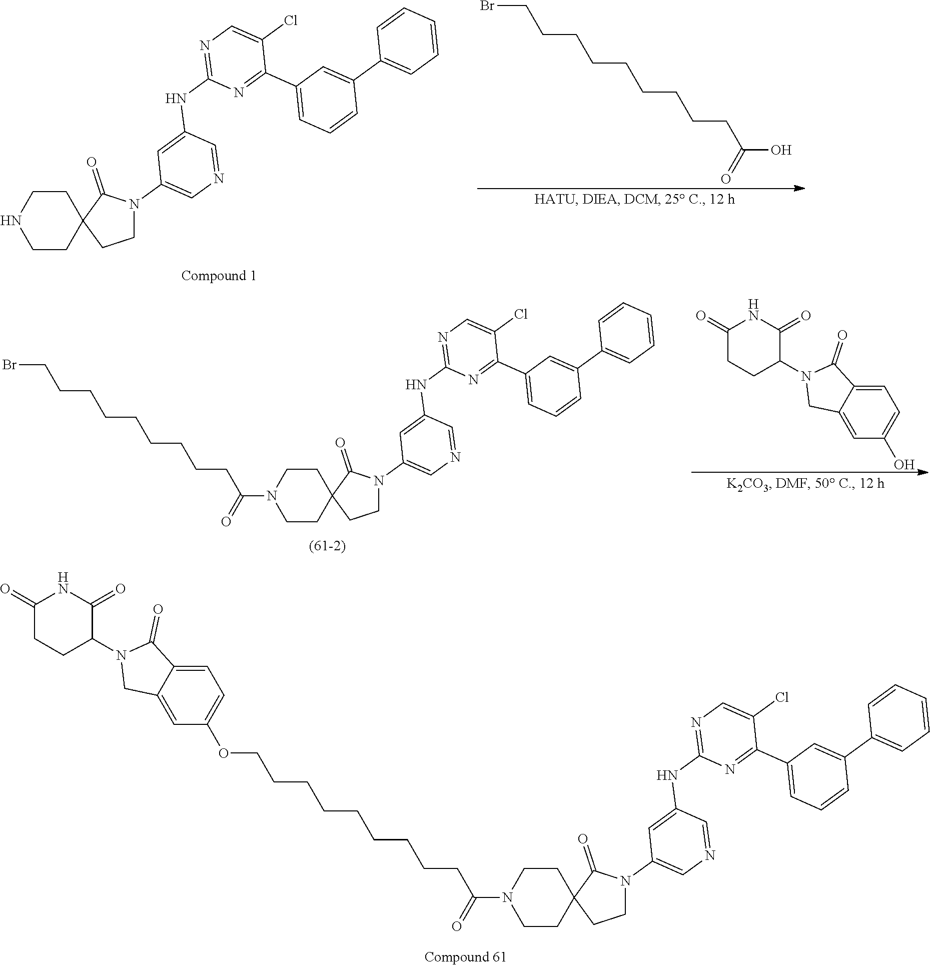 [SMILES: O=C1N(c2cncc(Nc3ncc(Cl)c(-c4cccc(-c5ccccc5)c4)n3)c2)CCC12CCNCC2 ; O=C(O)CCCCCCCCCBr ; O=C(CCCCCCCCCBr)N1CCC2(CC1)CCN(c1cncc(Nc3ncc(Cl)c(-c4cccc(-c5ccccc5)c4)n3)c1)C2=O ; O=C1CCC(N2Cc3cc(O)ccc3C2=O)C(=O)N1 ; O=C1CCC(N2Cc3cc(OCCCCCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1]

Synthesis of 2-(5-((4-([1,1′-biphenyl]-3-yl)-5-chloropyrimidin-2-yl)amino)pyridin-3-yl)-8-(10-bromodecanoyl)-2,8-diazaspiro[4.5]decan-1-one (61-2). A mixture of 10-bromodecanoic acid (108 mg, 431 μmol, 1.10 eq), 2-(5-((4-([1,1′-biphenyl]-3-yl)-5-chloropyrimidin-2-yl)amino)pyridin-3-yl)-2,8-diazaspiro[4.5]decan-1-one (Compound 1) (200 mg, 391 μmol, 1.00 eq), HATU (164 mg, 431 μmol, 1.10 eq) and DIEA (152 mg, 1.17 mmol, 205 μL, 3.00 eq) in DCM (3 mL) was stirred at 25° C. for 12 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely. Several new peaks were shown on LCMS and 54% of desired compound was detected. The reaction mixture was diluted with H₂O (2 mL) and extracted with EtOAc (2 mL*3). The combined organic layers were dried over Na₂SO₄, filtered and concentrated under reduced pressure to give a residue. The residue was purified by prep-TLC (SiO₂, Ethyl acetate:MeOH=10:1) to give the title compound (61-2) (220 mg, 278 μmol, 71.0% yield, 94.0% purity) as a white solid. LCMS (ES⁺): m/z 743.2 [M+H]⁺.

Synthesis of 3-(5-((10-(2-(5-((4-([1,1′-biphenyl]-3-yl)-5-chloropyrimidin-2-yl)amino)pyridin-3-yl)-1-oxo-2,8-diazaspiro[4.5]decan-8-yl)-10-oxodecyl)oxy)-1-oxoisoindolin-2-yl)piperidine-2,6-dione (Compound 61). A mixture of 3-(5-hydroxy-1-oxo-isoindolin-2-yl)piperidine-2,6-dione (90.9 mg, 349 μmol, 1.30 eq), 2-(5-((4-([1,1′-biphenyl]-3-yl)-5-chloropyrimidin-2-yl)amino)pyridin-3-yl)-8-(10-bromodecanoyl)-2,8-diazaspiro[4.5]decan-1-one (61-2) (200 mg, 269 μmol, 1.00 eq) and K₂CO₃ (111 mg, 806 μmol, 3.00 eq) in DMF (3 mL) was stirred at 50° C. for 12 h. LCMS showed 2-(5-((4-([1,1′-biphenyl]-3-yl)-5-chloropyrimidin-2-yl)amino)pyridin-3-yl)-8-(10-bromodecanoyl)-2,8-diazaspiro[4.5]decan-1-one (61-2) was consumed completely. Several new peaks were shown on LCMS and desired compound was detected. The reaction mixture was concentrated to give a residue. The residue was purified by prep-HPLC (column: Phenomenex luna C18 80*40 mm*3 um; mobile phase: [water (FA)-ACN]; B %: 30%-70%, 8 min) to give the title compound (Compound 61) (40.9 mg, 41.3 μmol, 15.4% yield, 93.2% purity) as a white solid. LCMS (ES⁺): m/z 923.3 [M+H]⁺.

### Example 62. Synthesis of 3-(5-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9-oxononyl)oxy)-1-oxoisoindolin-2-yl)piperidine-2,6-dione (Compound 62)

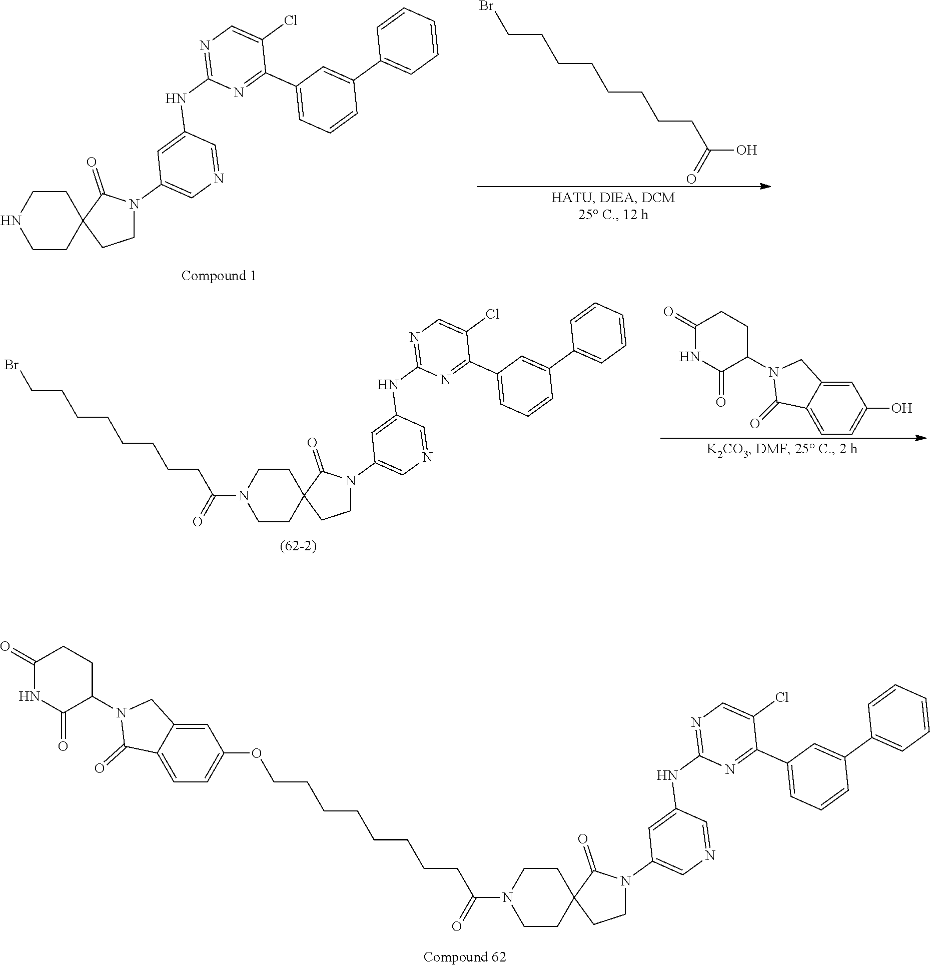

Synthesis of 2-(5-((4-([1,1′-biphenyl]-3-yl)-5-chloropyrimidin-2-yl)amino)pyridin-3-yl)-8-(9-bromopropanoyl)-2,8-diazaspiro[4.5]decan-1-one (62-2). A mixture of 9-bromononanoic acid (102 mg, 431 μmol, 1.10 eq), 2-(5-((4-([1,1′-biphenyl]-3-yl)-5-chloropyrimidin-2-yl)amino)pyridin-3-yl)-2,8-diazaspiro[4.5]decan-1-one (Compound 1) (200 mg, 391 μmol, 1.00 eq), HATU (164 mg, 431 μmol, 1.10 eq) and DIEA (152 mg, 1.17 mmol, 205 μL, 3.00 eq) in DCM (3 mL) was stirred at 25° C. for 12 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely. Several new peaks were shown on LCMS and 50% of desired compound was detected. The reaction mixture was diluted with H₂O (2 mL) and extracted with EtOAc (2 mL*3). The combined organic layers were dried over Na₂SO₄, filtered and concentrated under reduced pressure to give a residue. The residue was purified by prep-TLC (SiO₂, Ethyl acetate:MeOH=10:1) to give the title compound (62-2) (230 mg, 299 μmol, 76.5% yield, 95.0% purity) as a white solid. LCMS (ES⁺): m/z 729.3 [M+H]⁺.

Synthesis of 3-(5-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9-oxononyl)oxy)-1-oxoisoindolin-2-yl)piperidine-2,6-dione (Compound 62). A mixture of 3-(5-hydroxy-1-oxo-isoindolin-2-yl)piperidine-2,6-dione (102 mg, 392 μmol, 1.30 eq), 2-(5-((4-([1,1′-biphenyl]-3-yl)-5-chloropyrimidin-2-yl)amino)pyridin-3-yl)-8-(9-bromopropanoyl)-2,8-diazaspiro[4.5]decan-1-one (62-2) (220 mg, 301 μmol, 1.00 eq) and K₂CO₃ (125 mg, 904 μmol, 3.00 eq) in DMF (3 mL) was stirred at 50° C. for 12 h. LCMS showed 2-(5-((4-([1,1′-biphenyl]-3-yl)-5-chloropyrimidin-2-yl)amino)pyridin-3-yl)-8-(9-bromopropanoyl)-2,8-diazaspiro[4.5]decan-1-one (62-2) was consumed completely. Several new peaks were shown on LMS and desired compound was detected. The reaction mixture was concentrated to give a residue. The residue was purified by prep-HPLC (column: Phenomenex luna C18 80*40 mm*3 um; mobile phase: [water (FA)-ACN]; B %: 30%-70%, 8 min) to give the title compound (Compound 62) (40.9 mg, 41.9 μmol, 13.9% yield, 93.2% purity) as a white solid. LCMS (ES⁺): m/z 909.3 [M+H]⁺.

### Example 63. Synthesis of 3-(5-((8-(2-(5-((4-([1,1′-biphenyl]-3-y)-5-chloropyrimidin-2-yl)amino)pyridin-3-yl)-1-oxo-2,8-diazaspiro[4.5]decan-8-yl)-8-oxooctyl)oxy)-1-oxoisoindolin-2-yl)piperidine-2,6-dione (Compound 63)

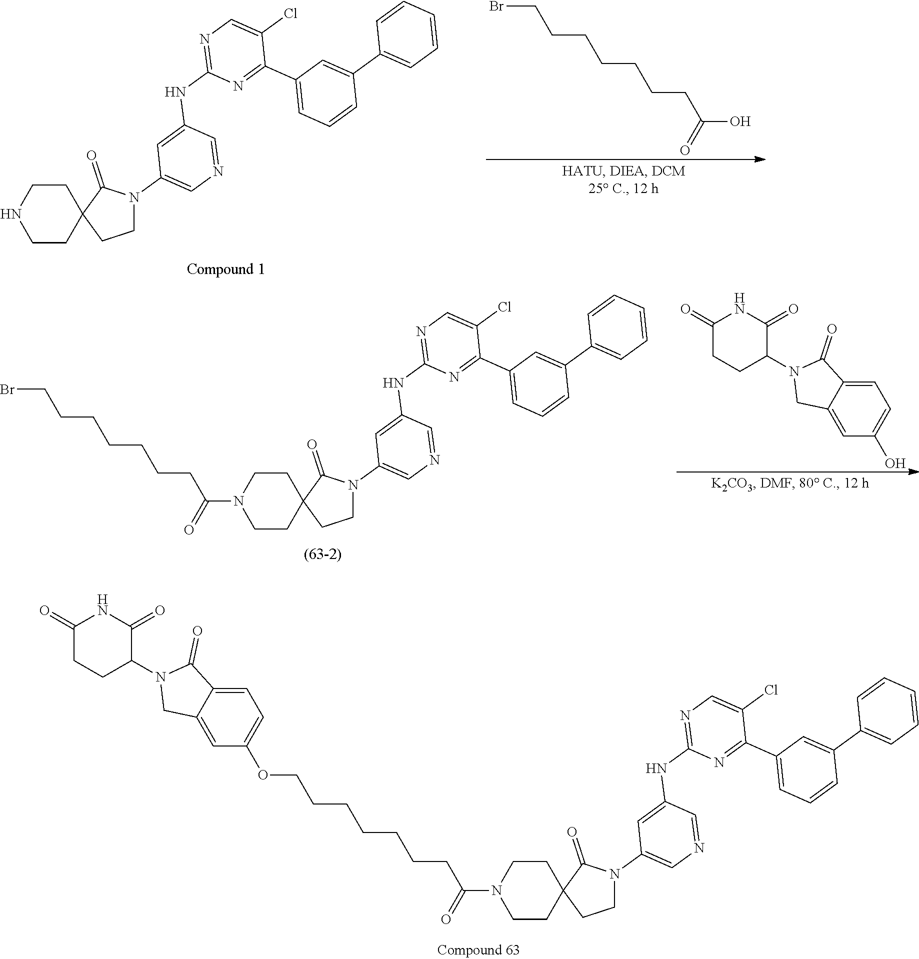

Synthesis of 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (63-2). A mixture of 8-bromooctanoic acid (96.1 mg, 431 μmol, 1.10 eq), 2-(5-((4-([1,1′-biphenyl]-3-yl)-5-chloropyrimidin-2-yl)amino)pyridin-3-yl)-2,8-diazaspiro[4.5]decan-1-one (Compound 1) (200 mg, 391 μmol, 1.00 eq), HATU (164 mg, 431 μmol, 1.10 eq) and DIEA (152 mg, 1.17 mmol, 205 μL, 3.00 eq) in DCM (3 mL) was stirred at 25° C. for 12 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely. Several new peaks were shown on LCMS and 57% of desired compound was detected. The reaction mixture was diluted with H₂O (2 mL) and extracted with EtOAc (2 mL*3). The combined organic layers were dried over Na₂SO₄, filtered and concentrated under reduced pressure to give a residue. The residue was purified by prep-TLC (SiO₂, Ethyl acetate:MeOH=10:1) to give the title compound (63-2) (250 mg, 342 μmol, 87.4% yield, 98.0% purity) as a white solid. LCMS (ES⁺): m/z 715.3 [M+H]⁺.

Synthesis of 3-(5-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)oxy)-1-oxoisoindolin-2-yl)piperidine-2,6-dione (Compound 63). A mixture of 3-(5-hydroxy-1-oxo-isoindolin-2-yl)piperidine-2,6-dione (142 mg, 545 μmol, 1.30 eq), 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (63-2) (300 mg, 419 μmol, 1.00 eq) and K₂CO₃ (174 mg, 1.26 mmol, 3.00 eq) in DMF (4 mL) was stirred at 80° C. for 12 h. LCMS showed 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (63-2) was consumed completely. Several new peaks were shown on LCMS and desired compound was detected. The reaction mixture was concentrated to give a residue. The residue was purified by prep-HPLC (column: Phenomenex luna C18 80*40 mm*3 um; mobile phase: [water (FA)-ACN]; B %: 30%-70%, 8 min) to give the title compound (Compound 63) (41.0 mg, 42.7 μmol, 10.2% yield, 93.2% purity) as a white solid. LCMS (ES⁺): m/z 895.3 [M+H]⁺.

### Example 64. Synthesis of 3-(5-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)-7-oxoheptyl)oxy)-1-oxoisoindolin-2-yl)piperidine-2,6-dione (Compound 64)

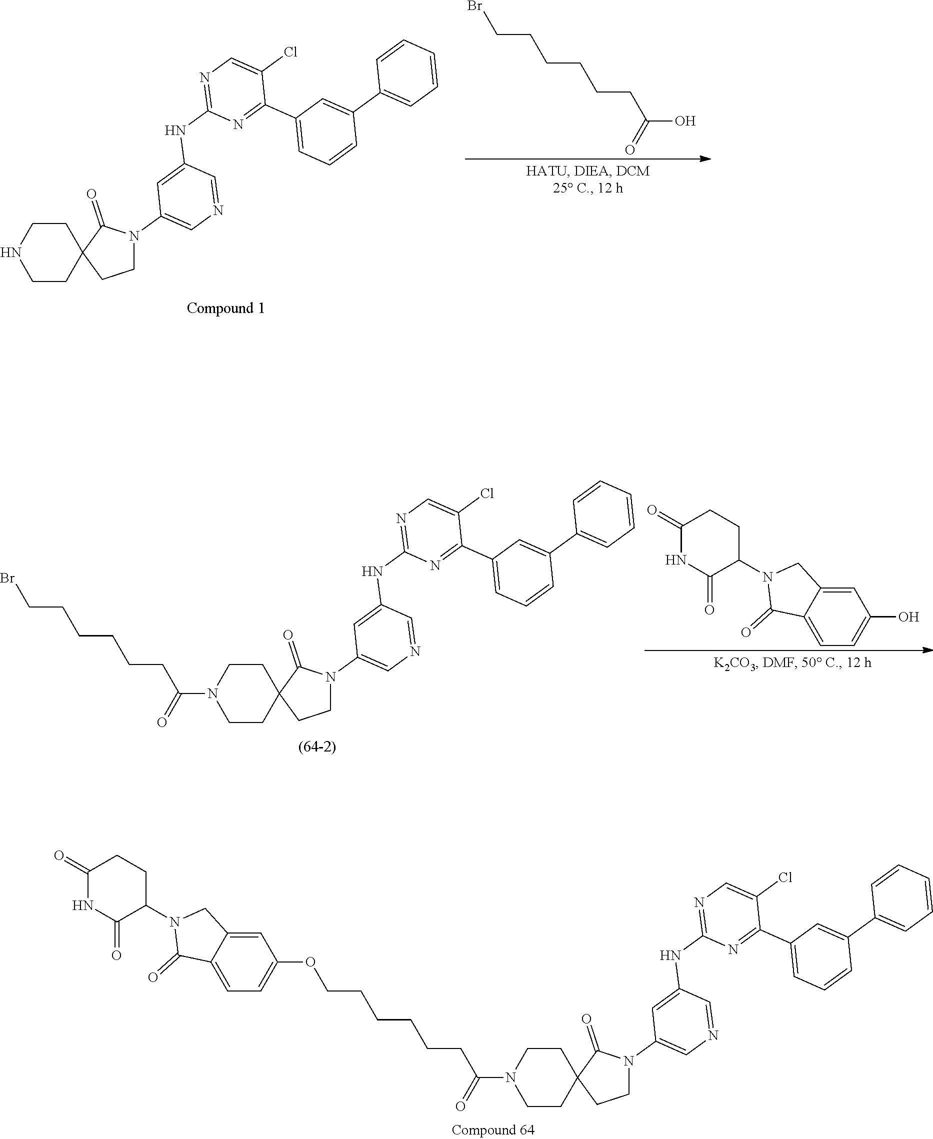

Synthesis of 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (64-2). A mixture of 7-bromoheptanoic acid (22.5 mg, 108 μmol, 1.10 eq), 2-(5-((4-([1,1′-biphenyl]-3-yl)-5-chloropyrimidin-2-yl)amino)pyridin-3-yl)-2,8-diazaspiro[4.5]decan-1-one (Compound 1 (50.0 mg, 97.8 μmol, 1.00 eq), HATU (40.9 mg, 108 μmol, 1.10 eq) and DIEA (37.9 mg, 294 μmol, 51.1 μL, 3.00 eq) in DMF (1 mL) was stirred at 25° C. for 12 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely. Several new peaks were shown on LCMS and 55% of desired compound was detected. The reaction mixture was diluted with H₂O (3 mL) and extracted with EtOAc (2 mL*3). The combined organic layers were dried over Na₂SO₄, filtered and concentrated under reduced pressure to give a residue. The residue was purified by prep-TLC (SiO₂, Ethyl acetate:MeOH=10:1) to give the title compound (64-2) (50.0 mg, 64.8 μmol, 66.2% yield, 91.0% purity) as a yellow oil. LCMS (ES⁺): m/z 701.1 [M+H]⁺.

Synthesis of 3-(5-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)-7-oxoheptyl)oxy)-1-oxoisoindolin-2-yl)piperidine-2,6-dione (Compound 64). A mixture of 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (64-2) (40.0 mg, 57.0 μmol, 1.00 eq), 3-(5-hydroxy-1-oxo-isoindolin-2-yl)piperidine-2,6-dione (14.8 mg, 57.0 μmol, 1.00 eq) and K₂CO₃ (23.6 mg, 171 μmol, 3.00 eq) in DMF (2 mL) was stirred at 50° C. for 12 h. LCMS showed 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (64-2) was consumed completely. Several new peaks were shown on LCMS and desired compound was detected. The reaction mixture was concentrated to give a residue. The residue was purified by prep-HPLC (column: Phenomenex luna C18 80*40 mm*3 um; mobile phase: [water (FA)-ACN]; B %: 30%-70%, 8 min) to give the title compound (Compound 64) (6.00 mg, 6.60 μmol, 11.6% yield, 97.0% purity) as a white solid. LCMS (ES⁺): m/z 881.2 [M+H]⁺.

### Example 65. Synthesis of N-(5-((4-([1,1′-biphenyl]-3-yl)-5-chloropyrimidin-2-yl)amino)pyridin-3-yl)-5-(4-(3-((2-(2,6-dioxopiperidin-3-yl)-1,3-dioxoisoindolin-4-yl)oxy)propyl)piperazin-1-yl)pentanamide (Compound 65)

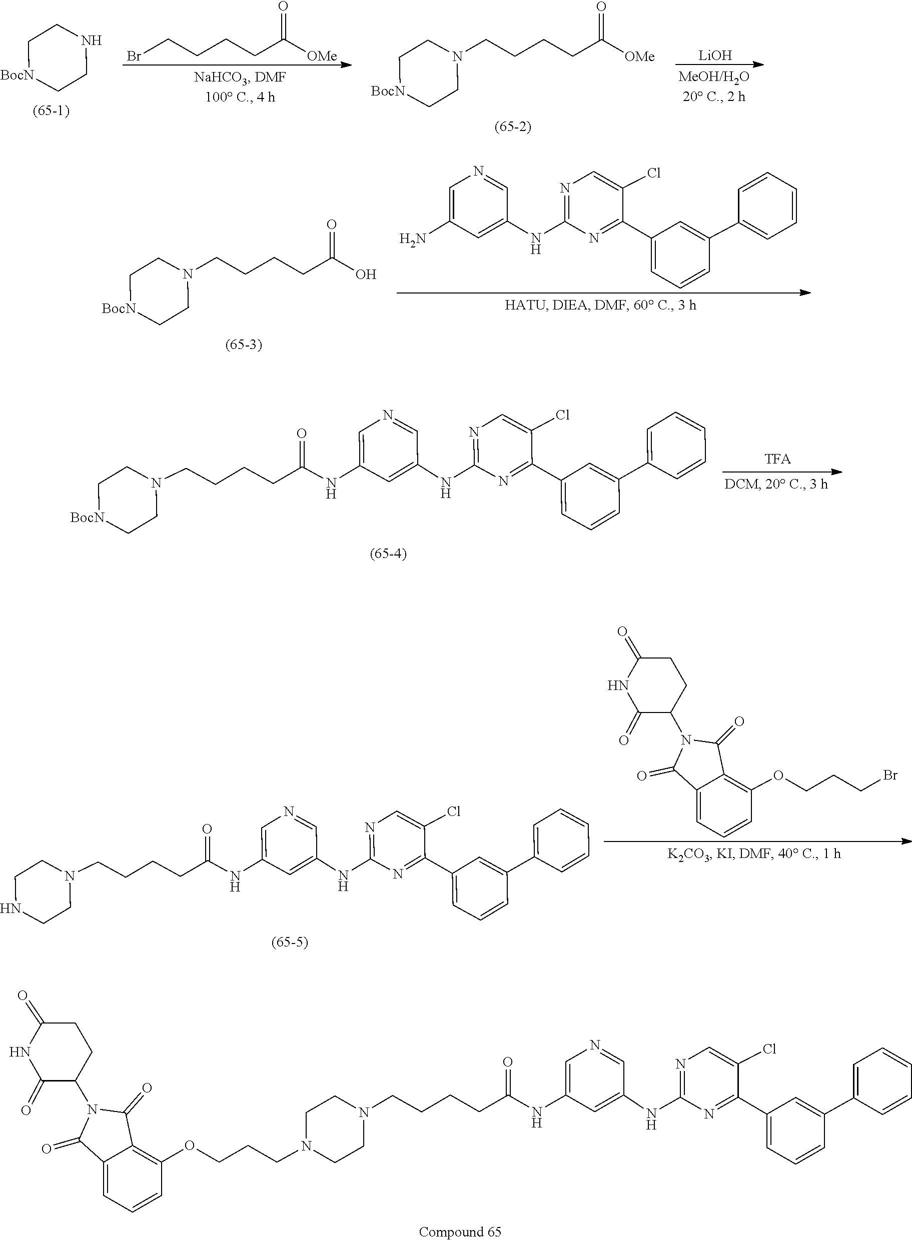

Synthesis of tert-butyl 4-(5-methoxy-5-oxopentyl)piperazine-1-carboxylate (65-2). A mixture of tert-butyl piperazine-1-carboxylate (65-1) (5.00 g, 26.9 mmol, 1.00 eq), methyl 5-bromopentanoate (5.24 g, 26.9 mmol, 3.85 mL, 1.00 eq) and NaHCO₃ (6.77 g, 80.5 mmol, 3.13 mL, 3.00 eq) in DMF (50 mL) was stirred at 100° C. for 4 h. LCMS showed desired m/z was detected. Water (5 mL) was added to the mixture at 20° C. The reaction mixture was diluted with H₂O (50 mL) and extracted with ethyl acetate (50 mL*3). The combined organic layers were dried over Na₂SO₄, filtered and concentrated under reduced pressure to give the title compound (65-2) (9.00 g, crude) as a brown oil. LCMS (ES⁺): m/z 301.2 [M+H]⁺.

Synthesis of 5-(4-(tert-butoxycarbonyl)piperazin-1-yl)pentanoic acid (65-3). A mixture of tert-butyl 4-(5-methoxy-5-oxopentyl)piperazine-1-carboxylate (65-2) (5.00 g, 16.6 mmol, 1.00 eq), LiOH (1.20 g, 49.9 mmol, 3.00 eq) in MeOH (25 mL) and H₂O (5 mL) was stirred at 20° C. for 2 h under N₂ atmosphere. LCMS showed desired m/z was detected. The reaction mixture was concentrated in vacuo to remove MeOH, the aqueous phase was diluted with H₂O (50 mL), acidified with 1 M of HCl to pH=5, and extracted with ethyl acetate (50 mL*3). The combined organic layers concentrated under reduced pressure to give the title compound (65-3) (5.00 g, crude) as a yellow solid. LCMS (ES⁺): m/z 287.1 [M+H]⁺.

Synthesis of tert-butyl 4-(5-((5-((4-([1,1′-biphenyl]-3-yl)-5-chloropyrimidin-2-yl)amino)pyridin-3-yl)amino)-5-oxopentyl)piperazine-1-carboxylate (65-4). To a solution of 5-(4-(tert-butoxycarbonyl)piperazin-1-yl)pentanoic acid (65-3) (500 mg, 1.75 mmol, 1.00 eq) and N3-[5-chloro-4-(3-phenylphenyl)pyrimidin-2-yl]pyridine-3,5-diamine (587 mg, 1.57 mmol, 0.900 eq) (prepared as described in Example 58) in DMF (10 mL) were added DIEA (677 mg, 5.24 mmol, 912 μL, 3.00 eq) and HATU (797 mg, 2.10 mmol, 1.20 eq). The mixture was stirred at 60° C. for 3 h. LCMS showed desired m/z was detected. The reaction mixture was diluted with H₂O (10 mL) and extracted with ethyl acetate (10 mL*3). The combined organic layers concentrated under reduced pressure to give the title compound (65-4) (1.12 g, crude) as a brown oil. LCMS (ES⁺): m/z 642.2 [M+H]⁺.

Synthesis of N-(5-((4-([1,1′-biphenyl]-3-yl)-5-chloropyrimidin-2-yl)amino)pyridin-3-yl)-5-(piperazin-1-yl)pentanamide (65-5). To a solution of tert-butyl 4-(5-((5-((4-([1,1′-biphenyl]-3-yl)-5-chloropyrimidin-2-yl)amino)pyridin-3-yl)amino)-5-oxopentyl)piperazine-1-carboxylate (65-4) (1.12 g, 1.74 mmol, 1.00 eq) in DCM (10 mL) was added TFA (3.08 g, 27.0 mmol, 2 mL, 15.5 eq). The mixture was stirred at 20° C. for 3 h. LCMS showed desired m/z was detected. The reaction mixture was concentrated under reduced pressure to give a residue. The residue was purified by prep-HPLC (column: Phenomenex Luna C18 75*30 mm*3 um; mobile phase: [water (FA)-ACN]; B %: 10%-40%, 8 min) to give the title compound (65-5) (400 mg, crude) as a white solid. LCMS (ES⁺): m/z 542.2 [M+H]⁺.

Synthesis of N-(5-((4-([1,1′-biphenyl]-3-yl)-5-chloropyrimidin-2-yl)amino)pyridin-3-yl)-5-(4-(3-((2-(2,6-dioxopiperidin-3-yl)-1,3-dioxoisoindolin-4-yl)oxy)propyl)piperazin-1-yl)pentanamide (Compound 65). To a solution of N-(5-((4-([1,1′-biphenyl]-3-yl)-5-chloropyrimidin-2-yl)amino)pyridin-3-yl)-5-(piperazin-1-yl)pentanamide (65-5) (200 mg, 369 μmol, 1.00 eq) and 4-(3-bromopropoxy)-2-(2,6-dioxo-3-piperidyl)isoindoline-1,3-dione (146 mg, 369 μmol, 1.00 eq) in DMF (3 mL) were added K₂CO₃ (153 mg, 1.11 mmol, 3.00 eq) and KI (30.6 mg, 184 μmol, 0.500 eq). The mixture was stirred at 40° C. for 1 h. LCMS showed desired m/z was detected. The reaction mixture was concentrated under reduced pressure to give a residue. The residue was purified by prep-HPLC (column: Phenomenex Luna C18 75*30 mm*3 um; mobile phase: [water (FA)-ACN]; B %: 20%-60%, 8 min) to give the title compound (Compound 65) (25.0 mg, 29.2 μmol, 7.91% yield) as a white solid. LCMS (ES⁺): m/z 856.3 [M+H]⁺.

### Example 66. Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1-oxoisoindolin-4-yl)oxy)acetamido)decanamide (Compound 66)

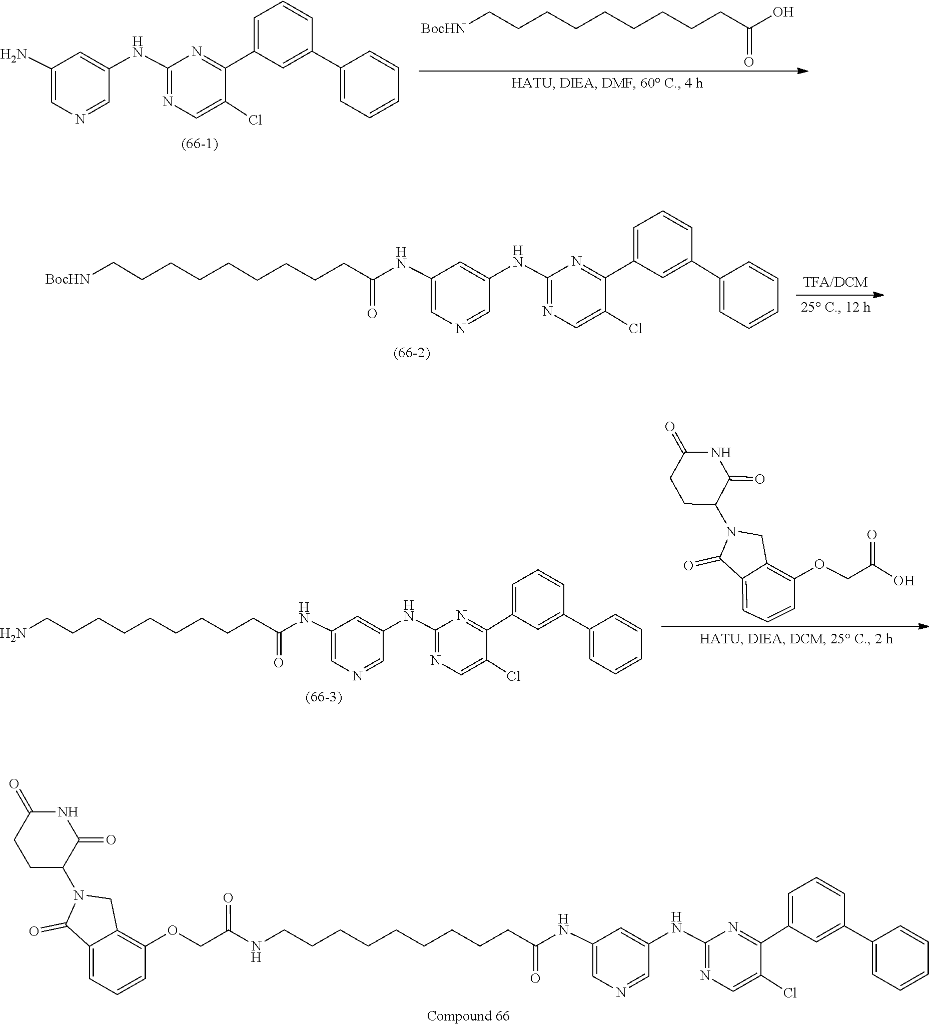 [SMILES: Nc1cncc(Nc2ncc(Cl)c(-c3cccc(-c4ccccc4)c3)n2)c1 ; CC(C)(C)OC(=O)NCCCCCCCCCC(=O)O ; CC(C)(C)OC(=O)NCCCCCCCCCC(=O)Nc1cncc(Nc2ncc(Cl)c(-c3cccc(-c4ccccc4)c3)n2)c1 ; NCCCCCCCCCC(=O)Nc1cncc(Nc2ncc(Cl)c(-c3cccc(-c4ccccc4)c3)n2)c1 ; O=C(O)COc1cccc2c1CN(C1CCC(=O)NC1=O)C2=O ; O=C(COc1cccc2c1CN(C1CCC(=O)NC1=O)C2=O)NCCCCCCCCCC(=O)Nc1cncc(Nc2ncc(Cl)c(-c3cccc(-c4ccccc4)c3)n2)c1]

Synthesis of tert-butyl (10-((5-((4-([1,1′-biphenyl]-3-yl)-5-chloropyrimidin-2-yl)amino)pyridin-3-yl)amino)-10-oxodecyl)carbamate (66-2). To a solution of N3-(4-([1,1′-biphenyl]-3-yl)-5-chloropyrimidin-2-yl)pyridine-3,5-diamine (66-1) (prepared as described in Example 58) (600 mg, 1.60 mmol, 1.00 eq) and 10-(tert-butoxycarbonylamino)decanoic acid (461 mg, 1.60 mmol, 1.00 eq) in DMF (10 mL) were added HATU (915 mg, 2.41 mmol, 1.50 eq) and DIEA (1.24 g, 9.63 mmol, 1.68 mL, 6.00 eq) at 25° C. The mixture was stirred at 60° C. for 2 h. LCMS showed ˜32.4% of N3-(4-([1,1′-biphenyl]-3-yl)-5-chloropyrimidin-2-yl)pyridine-3,5-diamine (66-1) was remaining and desired mass was detected. 10-(tert-butoxycarbonylamino)decanoic acid (460 mg) and HATU (915 mg) were added to the mixture at 25° C. Then the mixture was stirred at 60° C. for 2 h. LCMS showed N3-(4-([1,1′-biphenyl]-3-yl)-5-chloropyrimidin-2-yl)pyridine-3,5-diamine (66-1) was consumed completely and the desired mass was detected. Water (10 mL) was added to the mixture at 20° C. Then the reaction mixture was extracted with EtOAc (8 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:3 to 0:1) to give the title compound (66-2) (1.20 g, 1.42 mmol, 88.2% yield, 75.9% purity) as an orange oil. LCMS (ES⁺): m/z 643.3 [M+H]⁺.

Synthesis of N-(5-((4-([1,1′-biphenyl]-3-yl)-5-chloropyrimidin-2-yl)amino)pyridin-3-yl)-10-aminodecanamide (66-3). To a solution of tert-butyl (10-((5-((4-([1,1′-biphenyl]-3-yl)-5-chloropyrimidin-2-yl)amino)pyridin-3-yl)amino)-10-oxodecyl)carbamate (66-2) (300 mg, 466 μmol, 1.00 eq) in DCM (3 mL) was added TFA (1.54 g, 13.5 mmol, 1.00 mL, 29.0 eq) at 25° C. The mixture was stirred at 25° C. for 12 h. LCMS showed tert-butyl (10-((5-((4-([1,1′-biphenyl]-3-yl)-5-chloropyrimidin-2-yl)amino)pyridin-3-yl)amino)-10-oxodecyl)carbamate (66-2) was consumed completely and the desired mass was detected. The reaction mixture was concentrated under reduced pressure to remove solvent to give the title compound (66-3) (300 mg, crude, TFA) as a yellow oil. LCMS (ES⁺): m/z 543.3 [M+H]⁺.

Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1-oxoisoindolin-4-yl)oxy)acetamido)decanamide (Compound 66). To a solution of 2-[2-(2,6-dioxo-3-piperidyl)-1-oxo-isoindolin-4-yl]oxyacetic acid (140 mg, 441 μmol, 1.00 eq) in DCM (5 mL) were added HATU (185 mg, 485 μmol, 1.10 eq), N-(5-((4-([1,1′-biphenyl]-3-yl)-5-chloropyrimidin-2-yl)amino)pyridin-3-yl)-10-aminodecanamide (66-3) (290 mg, 441 μmol, 1.00 eq, TFA) and DIEA (171 mg, 1.32 mmol, 231 μL, 3.00 eq) at 25° C. The mixture was stirred at 25° C. for 2 h. LCMS showed N-(5-((4-([1,1′-biphenyl]-3-yl)-5-chloropyrimidin-2-yl)amino)pyridin-3-yl)-10-aminodecanamide (66-3) was consumed completely and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH₄HCO₃)-ACN]; B %: 50%-80%, 8 min) to give the title compound (Compound 66) (26.1 mg, 30.1 μmol, 6.81% yield, 97.2% purity) as a white solid. LCMS (ES⁺): m/z 843.3 [M+H]⁺.

### Example 67. Synthesis of N-(5-((4-([1,1′-biphenyl]-3-yl)-5-chloropyrimidin-2-yl)amino)pyridin-3-yl)-8-(4-(3-((2-(2,6-dioxopiperidin-3-yl)-1,3-dioxoisoindolin-4-yl)oxy)propyl)piperazin-1-yl)octanamide (Compound 67)

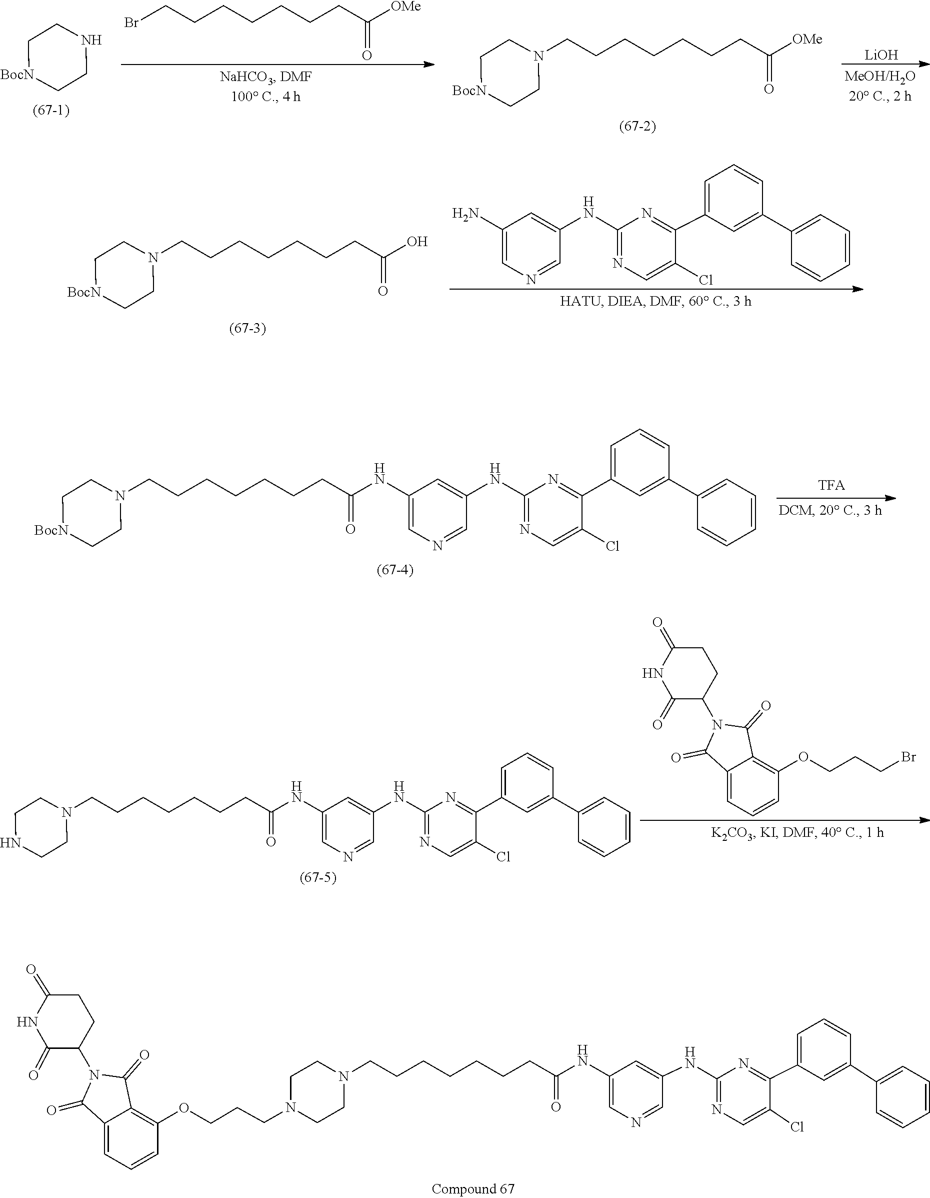

Synthesis of tert-butyl 4-(8-methoxy-8-oxooctyl)piperazine-1-carboxylate (67-2). A mixture of tert-butyl piperazine-1-carboxylate (67-1) (3.71 g, 19.9 mmol, 1.00 eq), ethyl 8-bromooctanoate (5.00 g, 19.9 mmol, 1.00 eq) and NaHCO₃ (5.02 g, 59.7 mmol, 2.32 mL, 3.00 eq) in DMF (40 mL) was stirred at 100° C. for 4 h. LCMS showed desired m/z was detected. The reaction mixture was diluted with H₂O (40 mL) and extracted with ethyl acetate (20 mL*3). The combined organic layers were dried over Na₂SO₄, filtered and concentrated under reduced pressure to give the title compound (67-2) (7.00 g, crude) as a yellow oil. LCMS (ES⁺): m/z 343.2 [M+H]⁺.

Synthesis of 8-(4-(tert-butoxycarbonyl)piperazin-1-yl)octanoic acid (67-3). To a solution of tert-butyl 4-(8-methoxy-8-oxooctyl)piperazine-1-carboxylate (67-2) (3.50 g, 9.82 mmol, 1.00 eq) in MeOH (25 mL) and H₂O (5 mL) was added LiOH (705 mg, 29.5 mmol, 3.00 eq). The mixture was stirred at 20° C. for 2 h. LCMS showed desired m/z was detected. The reaction mixture was concentrated in vacuo to remove MeOH, the aqueous phase was diluted with H₂O (40 mL), acidified with 1 M of HCl to pH=5, and extracted with ethyl acetate (20 mL*3). The combined organic layers were dried over Na₂SO₄, filtered and concentrated under reduced pressure to give the title compound (67-3) (1.25 g, crude) as a white solid. LCMS (ES⁺): m/z 329.2 [M+H]⁺.

Synthesis of tert-butyl 4-(8-((5-((4-([1,1′-biphenyl]-3-yl)-5-chloropyrimidin-2-yl)amino)pyridin-3-yl)amino)-8-oxooctyl)piperazine-1-carboxylate (67-4). To a solution of 8-(4-(tert-butoxycarbonyl)piperazin-1-yl)octanoic acid (67-3) (500 mg, 1.52 mmol, 1.00 eq) and N3-[5-chloro-4-(3-phenylphenyl)pyrimidin-2-yl]pyridine-3,5-diamine (512 mg, 1.37 mmol, 0.900 eq) (prepared as described in Example 58) in DMF (10 mL) were added DIEA (590 mg, 4.57 mmol, 795 μL, 3.00 eq) and HATU (695 mg, 1.83 mmol, 1.20 eq). The mixture was stirred at 60° C. for 3 h. LCMS showed desired m/z was detected. The reaction mixture was diluted with H₂O (10 mL) and extracted with ethyl acetate (10 mL*3). The combined organic layers were dried over Na₂SO₄, filtered and concentrated under reduced pressure to give the title compound (67-4) (1.04 g, crude) as a brown oil. LCMS (ES⁺): m/z 684.3 [M+H]⁺.

Synthesis of N-(5-((4-([1,1′-biphenyl]-3-yl)-5-chloropyrimidin-2-yl)amino)pyridin-3-yl)-8-(piperazin-1-yl)octanamide (67-5). To a solution of tert-butyl 4-(8-((5-((4-([1,1′-biphenyl]-3-yl)-5-chloropyrimidin-2-yl)amino)pyridin-3-yl)amino)-8-oxooctyl)piperazine-1-carboxylate (67-4) (1.09 g, 1.66 mmol, 1.00 eq) in DCM (10 mL) was added TFA (3.08 g, 27.0 mmol, 2.00 mL, 16.3 eq). The mixture was stirred at 20° C. for 3 h. LCMS showed desired m/z was detected. The reaction mixture was concentrated under reduced pressure to give a residue. The residue was purified by prep-HPLC (column: Phenomenex Luna C18 75*30 mm*3 um; mobile phase: [water (FA)-ACN]; B %: 15%-45%, 8 min to give the title compound (67-5) (600 mg, 1.08 mmol, 65.0% yield) as a white solid. LCMS (ES⁺): m/z 584.2 [M+H]⁺.

Synthesis of N-(5-((4-([1,1′-biphenyl]-3-yl)-5-chloropyrimidin-2-yl)amino)pyridin-3-yl)-8-(4-(3-((2-(2,6-dioxopiperidin-3-yl)-1,3-dioxoisoindolin-4-yl)oxy)propyl)piperazin-1-yl)octanamide (Compound 67). To a solution of N-(5-((4-([1,1′-biphenyl]-3-yl)-5-chloropyrimidin-2-yl)amino)pyridin-3-yl)-8-(piperazin-1-yl)octanamide (67-5) (200 mg, 342 μmol, 1.00 eq) and 5-(3-bromopropoxy)-2-(2,6-dioxo-3-piperidyl)isoindoline-1,3-dione (271 mg, 685 μmol, 2.00 eq) in DMF (10 mL) were added KI (28.4 mg, 171 μmol, 0.500 eq) and K₂CO₃ (142 mg, 1.03 mmol, 3.00 eq). The mixture was stirred at 40° C. for 1 h. LCMS showed desired m/z was detected. The reaction mixture was concentrated under reduced pressure to give a residue. The residue was purified by prep-HPLC (column: Phenomenex C18 75*30 mm*3 um; mobile phase: [water (FA)-ACN]; B %: 25%-45%, 8 min) to give the title compound (Compound 67) (25.0 mg, 27.8 μmol, 8.13% yield) as a white solid. LCMS (ES⁺): m/z 898.4 [M+H]⁺.

### Example 68. Synthesis of N-(5-((4-([1,1′-biphenyl]-3-yl)-5-chloropyrimidin-2-yl)amino)pyridin-3-yl)-6-(4-(3-((2-(2,6-dioxopiperidin-3-yl)-1,3-dioxoisoindolin-4-yl)oxy)propyl)piperazin-1-yl)hexanamide (Compound 68)

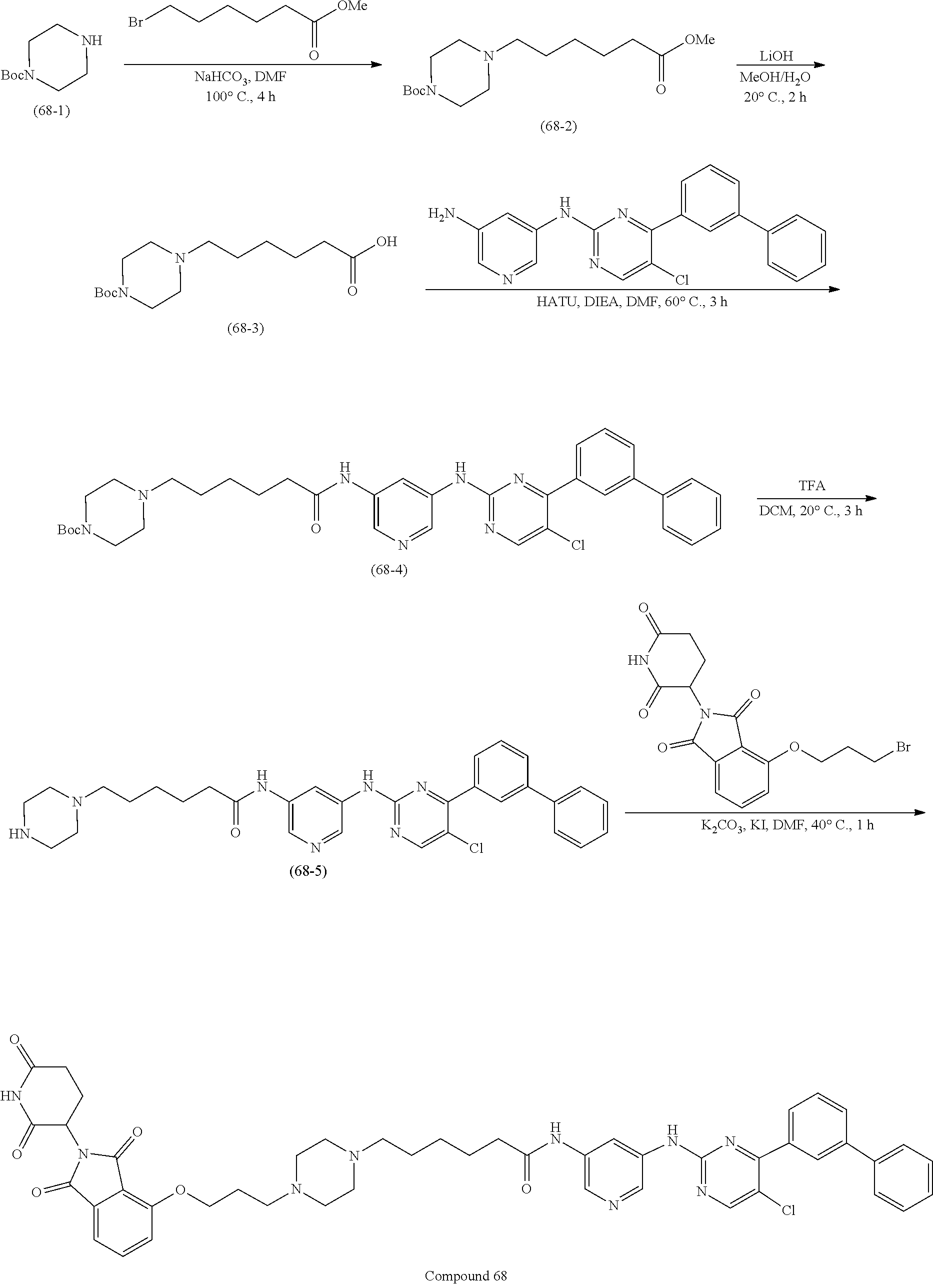

Synthesis of tert-butyl 4-(6-methoxy-6-oxohexyl)piperazine-1-carboxylate (68-2). A mixture of tert-butyl piperazine-1-carboxylate (68-1) (4.45 g, 23.9 mmol, 1.00 eq), methyl 6-bromohexanoate (5.00 g, 23.9 mmol, 1.00 eq) and NaHCO₃ (6.03 g, 71.7 mmol, 2.79 mL, 3.00 eq) in DMF (40 mL) was stirred at 100° C. for 4 h. LCMS showed desired m/z was detected. The reaction mixture was diluted with H₂O (40 mL) and extracted with ethyl acetate (20 mL*3). The combined organic layers were dried over Na₂SO₄, filtered and concentrated under reduced pressure to give the title compound (68-2) (7.00 g, crude) as a yellow oil. LCMS (ES⁺): m/z 315.2 [M+H]⁺.

Synthesis of 6-(4-(tert-butoxycarbonyl)piperazin-1-yl)hexanoic acid (68-3). To a mixture of tert-butyl 4-(6-methoxy-6-oxohexyl)piperazine-1-carboxylate (68-2) (3.49 g, 11.1 mmol, 1.00 eq) in MeOH (25 mL) and H₂O (5 mL) was added LiOH (797 mg, 33.3 mmol, 3.00 eq), and then the mixture was stirred at 20° C. for 2 h. LCMS showed desired m/z was detected. The reaction mixture was concentrated in vacuo to remove MeOH, the aqueous phase was diluted with H₂O (40 mL), acidified with 1 M of HCl to pH=5, and extracted with ethyl acetate (20 mL*3). The combined organic layers were dried over Na₂SO₄, filtered and concentrated under reduced pressure to give the title compound (68-3) (1.30 g, crude) as a white solid. LCMS (ES⁺): m/z 301.2 [M+H]⁺.

Synthesis of tert-butyl 4-(6-((5-((4-([1,1′-biphenyl]-3-yl)-5-chloropyrimidin-2-yl)amino)pyridin-3-yl)amino)-6-oxohexyl)piperazine-1-carboxylate (68-4). To a solution of 6-(4-(tert-butoxycarbonyl)piperazin-1-yl)hexanoic acid (68-3) (500 mg, 1.66 mmol, 1.00 eq) and N3-[5-chloro-4-(3-phenylphenyl)pyrimidin-2-yl]pyridine-3,5-diamine (560 mg, 1.50 mmol, 0.900 eq) (prepared as described in Example 58) in DMF (10 mL) were added DIEA (645 mg, 4.99 mmol, 870 μL, 3.00 eq) and HATU (759 mg, 2.00 mmol, 1.20 eq). The mixture was stirred at 60° C. for 3 h. LCMS showed desired m/z was detected. The reaction mixture was diluted with H₂O (10 mL) and extracted with ethyl acetate (10 mL*3). The combined organic layers were dried over Na₂SO₄, filtered and concentrated under reduced pressure to give the title compound (68-4) (1.09 g, crude) as a brown oil. LCMS (ES⁺): m/z 656.3 [M+H]⁺.

Synthesis of N-(5-((4-([1,1′-biphenyl]-3-yl)-5-chloropyrimidin-2-yl)amino)pyridin-3-yl)-6-(piperazin-1-yl)hexanamide (68-5). To a solution of tert-butyl 4-(6-((5-((4-([1,1′-biphenyl]-3-yl)-5-chloropyrimidin-2-yl)amino)pyridin-3-yl)amino)-6-oxohexyl)piperazine-1-carboxylate (68-4) (1.09 g, 1.66 mmol, 1.00 eq) in DCM (10 mL) was added TFA (3.08 g, 27.0 mmol, 2 mL, 16.3 eq). The mixture was stirred at 20° C. for 3 h. LCMS showed desired m/z was detected. The reaction mixture was concentrated under reduced pressure to give a residue. The residue was purified by prep-HPLC (column: Phenomenex Luna C18 75*30 mm*3 um; mobile phase: [water (FA)-ACN]; B %: 15%-45%, 8 min) to give the title compound (68-5) (600 mg, 1.08 mmol, 65.0% yield) as a white solid. LCMS (ES⁺): m/z 556.2 [M+H]⁺.

Synthesis of N-(5-((4-([1,1′-biphenyl]-3-yl)-5-chloropyrimidin-2-yl)amino)pyridin-3-yl)-6-(4-(3-((2-(2,6-dioxopiperidin-3-yl)-1,3-dioxoisoindolin-4-yl)oxy)propyl)piperazin-1-yl)hexanamide (Compound 68). To a solution of N-(5-((4-([1,1′-biphenyl]-3-yl)-5-chloropyrimidin-2-yl)amino)pyridin-3-yl)-6-(piperazin-1-yl)hexanamide (68-5) (200 mg, 360 μmol, 1.00 eq) and 5-(3-bromopropoxy)-2-(2,6-dioxo-3-piperidyl)isoindoline-1,3-dione (284 mg, 719 μmol, 2.00 eq) in DMF (10 mL) were added KI (29.9 mg, 180 μmol, 0.500 eq) and K₂CO₃ (149 mg, 1.08 mmol, 3.00 eq). The mixture was stirred at 40° C. for 1 h. LCMS showed desired m/z was detected. The reaction mixture was concentrated under reduced pressure to give a residue. The residue was purified by prep-HPLC (column: Phenomenex C18 75*30 mm*3 um; mobile phase: [water (FA)-ACN]; B %: 25%-45%, 8 min) to give the title compound (Compound 68) (25.0 mg, 28.7 μmol, 7.99% yield) as a white solid. LCMS (ES⁺): m/z 870.3 [M+H]⁺.

### Example 69. Synthesis of 3-(4-((10-(2-(5-((4-([1,1′-biphenyl]-3-yl)-5-chloropyrimidin-2-yl)amino)pyridin-3-yl)-1-oxo-2,8-diazaspiro[4.5]decan-8-yl)-10-oxodecyl)oxy)-1-oxoisoindolin-2-yl)piperidine-2,6-dione (Compound 69)

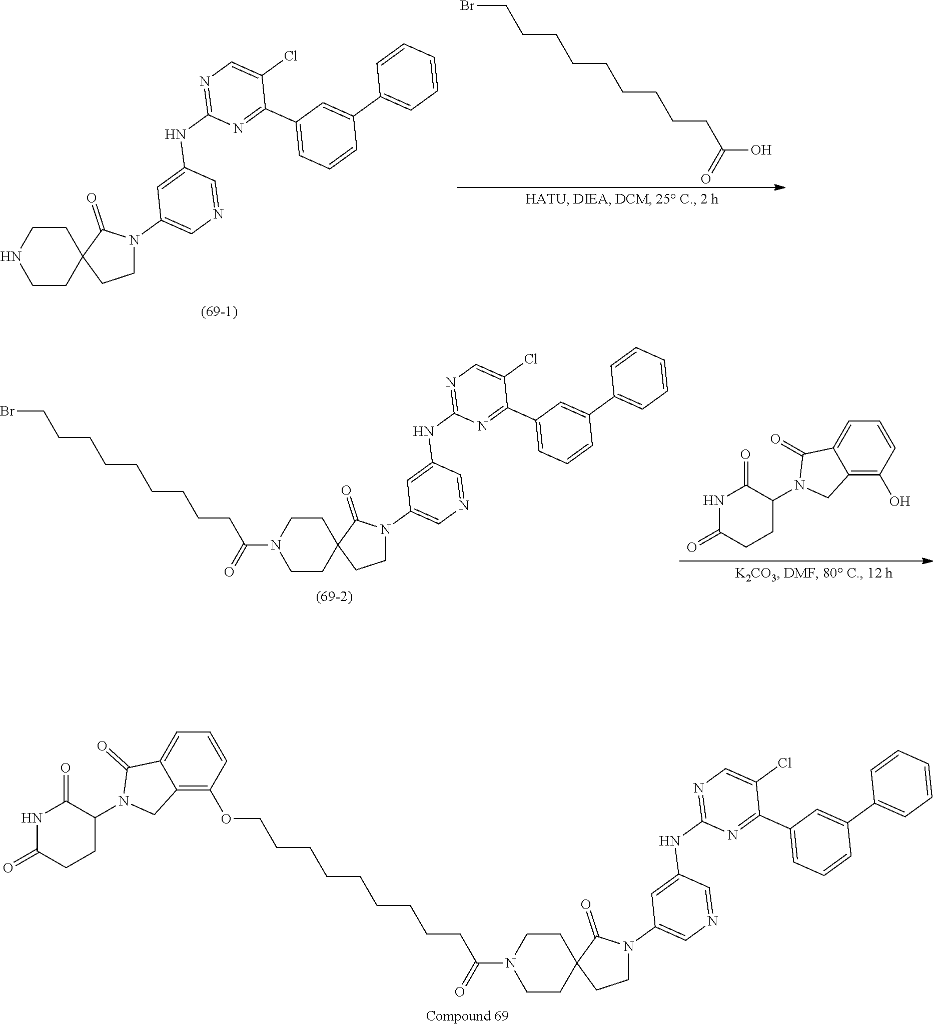 [SMILES: O=C1N(c2cncc(Nc3ncc(Cl)c(-c4cccc(-c5ccccc5)c4)n3)c2)CCC12CCNCC2 ; O=C(O)CCCCCCCCCBr ; O=C(CCCCCCCCCBr)N1CCC2(CC1)CCN(c1cncc(Nc3ncc(Cl)c(-c4cccc(-c5ccccc5)c4)n3)c1)C2=O ; O=C1CCC(N2Cc3c(O)cccc3C2=O)C(=O)N1 ; O=C1CCC(N2Cc3c(OCCCCCCCCCC(=O)N4CCC5(CC4)CCN(c4cncc(Nc6ncc(Cl)c(-c7cccc(-c8ccccc8)c7)n6)c4)C5=O)cccc3C2=O)C(=O)N1]

Synthesis of 2-(5-((4-([1,1′-biphenyl]-3-yl)-5-choropyrimidin-2-y)amino)pyridin-3-yl)-8-(10-bromodecanoyl)-2,8-diazaspiro[4.5]decan-1-one (69-2). To a solution of 2-(5-((4-([1,1′-biphenyl]-3-yl)-5-chloropyrimidin-2-yl)amino)pyridin-3-yl)-2,8-diazaspiro[4.5]decan-1-one (Compound 1) (300 mg, 480 μmol, 1.00 eq, TFA) in DCM (10 mL) were added HATU (201 mg, 528 μmol, 1.10 eq), DIEA (186 mg, 1.44 mmol, 251 μL, 3.00 eq) and 10-bromodecanoic acid (120 mg, 480 μmol, 1.00 eq) at 25° C. The mixture was stirred at 25° C. for 2 h. LCMS showed 2-(5-((4-([1,1′-biphenyl]-3-yl)-5-chloropyrimidin-2-yl)amino)pyridin-3-yl)-2,8-diazaspirop[4.5]decan-1-one (Compound 1) was consumed completely and the desired mass was detected. The reaction mixture was concentrated under reduced pressure to remove solvent. The residue was purified by prep-TLC (SiO₂, Ethyl acetate Methanol 10:1) to give the title compound (69-2) (200 mg, 269 μmol, 56.0% yield) as a colorless oil. LCMS (ES⁺): m/z 743.4 [M+H]⁺.

Synthesis of 3-(4-((0-(2-(5-((4-([1,1′-biphenyl]-3-yl)-5-chloropyrimidin-2-yl)amino)pyridin-3-yl)-1-oxo-2,8-diazaspiro[4.5]decan-8-yl)-10-oxodecyl)oxy)-1-oxoisoindolin-2-yl)piperidine-2,6-dione (Compound 69). To a solution of 2-(5-((4-([1,1′-biphenyl]-3-yl)-5-chloropyrimidin-2-yl)amino)pyridin-3-yl)-8-(10-bromodecanoyl)-2,8-diazaspiro[4.5]decan-1-one (69-2) (200 mg, 269 μmol, 1.00 eq) in DMF (5 mL) were added K₂C₃(111 mg, 806 μmol, 3.00 eq) and 3-(4-hydroxy-1-oxo-isoindolin-2-yl)piperidine-2,6-dione (69.9 mg, 269 μmol, 1.00 eq) at 25° C. The mixture was stirred at 80° C. for 12 h. LCMS showed 2-(5-((4-([1,1′-biphenyl]-3-yl)-5-chloropyrimidin-2-yl)amino)pyridin-3-yl)-8-(10-bromodecanoyl)-2,8-diazaspiro[4.5]decan-1-one (69-2) was consumed completely and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 69) (26.3 mg, 27.0 μmol, 10.0% yield, 94.7% purity) as a white solid. LCMS (ES⁺): m/z 923.5 [M+H]⁺.

### Example 70. Synthesis of 3-(4-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)oxy)-1-oxoisoindolin-2-yl)piperidine-2,6-dione (Compound 70)

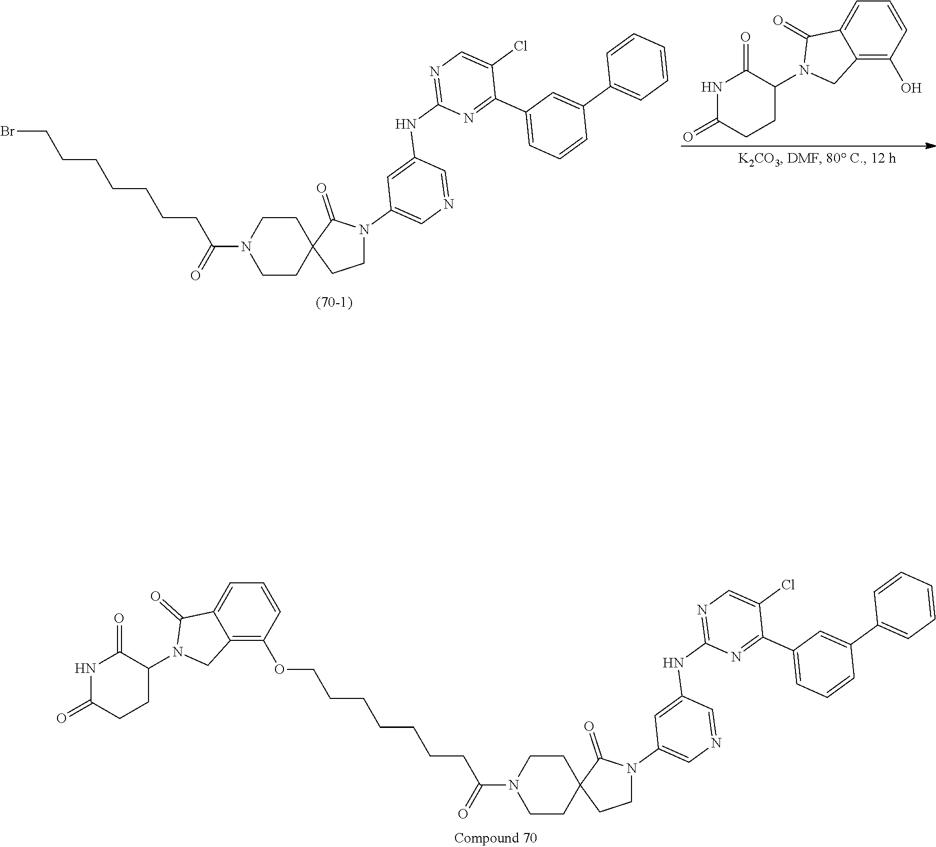 [SMILES: O=C(CCCCCCCBr)N1CCC2(CC1)CCN(c1cncc(Nc3ncc(Cl)c(-c4cccc(-c5ccccc5)c4)n3)c1)C2=O ; O=C1CCC(N2Cc3c(O)cccc3C2=O)C(=O)N1 ; O=C1CCC(N2Cc3c(OCCCCCCCC(=O)N4CCC5(CC4)CCN(c4cncc(Nc6ncc(Cl)c(-c7cccc(-c8ccccc8)c7)n6)c4)C5=O)cccc3C2=O)C(=O)N1]

Synthesis of 3-(4-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)oxy)-1-oxoisoindolin-2-yl)piperidine-2,6-dione (Compound 70). To a solution of 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (70-1) (as prepared in Example 63) (200 mg, 279 μmol, 1.00 eq) and 3-(4-hydroxy-1-oxoisoindolin-2-yl) piperidine-2, 6-dione (72.7 mg, 279 μmol, 1.00 eq) in DMF (2 mL) was added K₂CO₃ (116 mg, 838 μmol, 3.00 eq) at 15° C. The resulting mixture was stirred at 80° C. for 12 h. LCMS showed 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (70-1) was consumed and the desired mass was detected. The mixture was concentrated in vacuo. The mixture was purified by prep-HPLC (column: Phenomenex Luna C18 75*30 mm*3 um; mobile phase: [water (FA)-ACN]; B %: 30%-65%, 8 min) to give the title compound (Compound 70) (11.0 mg, 11.1 μmol, 3.99% yield, 90.62% purity) as a white solid. LCMS (ES⁺): m/z 895.4 [M+H]⁺.

### Example 71. Synthesis of 3-(4-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9oxononyl)oxy)-1-oxoisoindolin-2-yl)piperidine-2,6-dione (Compound 71)

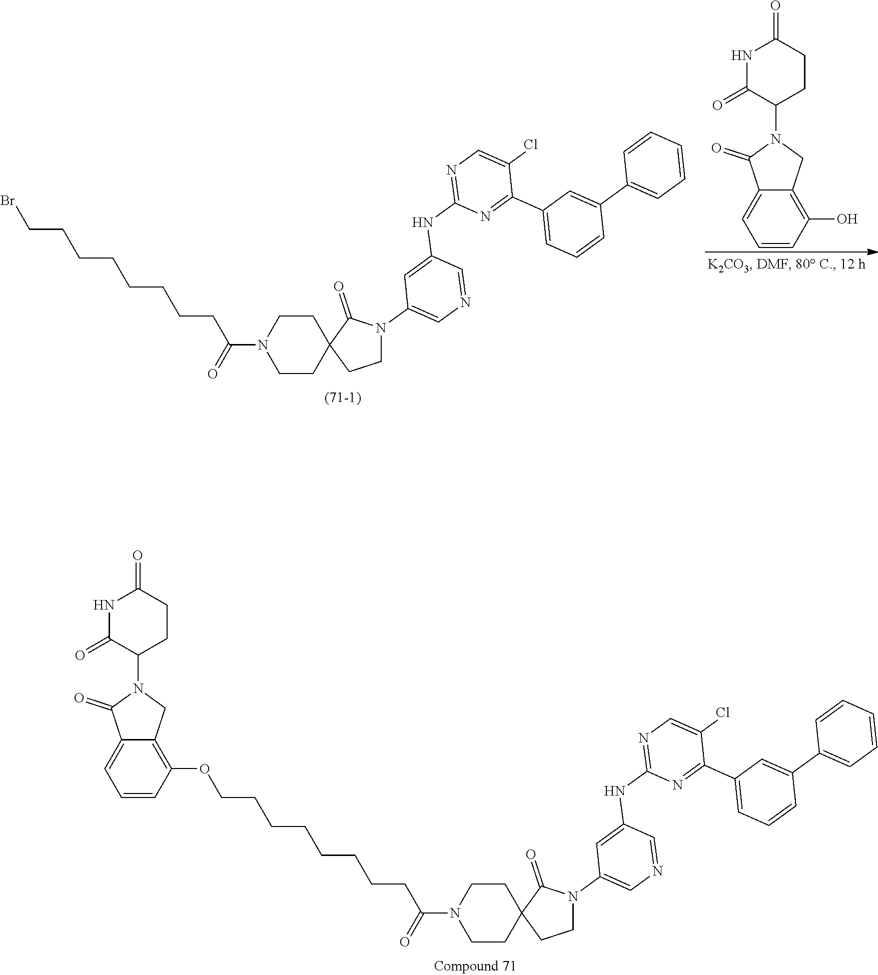

Synthesis of 3-(4-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9oxononyl)oxy)-1-oxoisoindolin-2-yl)piperidine-2,6-dione (Compound 71). To a solution of 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (71-1) (as prepared in Example 72) (240 mg, 329 μmol, 1.00 eq) in DMF (3 mL) were added 3-(4-hydroxy-1-oxoisoindolin-2-yl)piperidine-2,6-dione (85.5 mg, 329 μmol, 1.00 eq) and K₂CO₃ (136 mg, 986 μmol, 3.00 eq) at 20° C. The mixture was stirred at 80° C. for 12 h. LCMS showed that 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (71-1) was consumed completely and the desired mass was detected. The mixture was concentrated in vacuo. The residue was purified by prep-HPLC (column: Waters Xbridge BEH C18 100*30 mm*10 um; mobile phase: [water (NH₄HCO₃)-ACN]; B %: 45%-75%, 8 min) to give the title compound (Compound 71) (13.8 mg, 15.2 μmol, 4.62% yield, 100% purity) as a white solid. LCMS (ES⁺): m/z 909.2 [M+H]⁺.

### Example 72. Synthesis of 5-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9-oxononyl)oxy)-2-(2,6-dioxopiperidin-3-yl)isoindoline-1,3-dione (Compound 72)

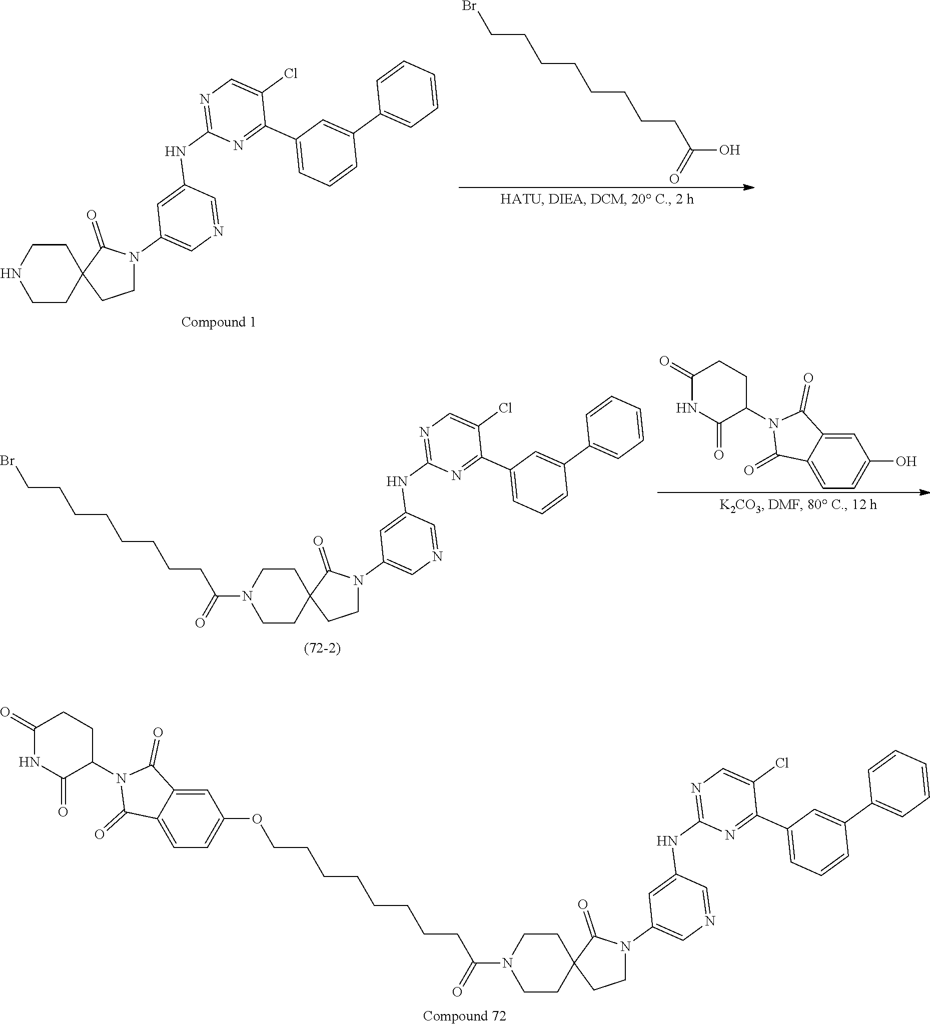 [SMILES: O=C1N(c2cncc(Nc3ncc(Cl)c(-c4cccc(-c5ccccc5)c4)n3)c2)CCC12CCNCC2 ; O=C(O)CCCCCCCCBr ; O=C(CCCCCCCCBr)N1CCC2(CC1)CCN(c1cncc(Nc3ncc(Cl)c(-c4cccc(-c5ccccc5)c4)n3)c1)C2=O ; O=C1CCC(N2C(=O)c3ccc(O)cc3C2=O)C(=O)N1 ; O=C1CCC(N2C(=O)c3ccc(OCCCCCCCCC(=O)N4CCC5(CC4)CCN(c4cncc(Nc6ncc(Cl)c(-c7cccc(-c8ccccc8)c7)n6)c4)C5=O)cc3C2=O)C(=O)N1]

Synthesis of 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (72-2). To a solution of 9-bromononanoic acid (75.9 mg, 320 μmol, 1.00 eq) in DCM (5 mL) were added HATU (134 mg, 352 μmol, 1.10 eq) and DIEA (128 mg, 992 μmol, 173 μL, 3.10 eq) at 20° C. Then 2-(5-((4-([1,1′-biphenyl]-3-yl)-5-chloropyrimidin-2-yl)amino)pyridin-3-yl)-2,8-diazaspiro[4.5]decan-1-one (Compound 1) (200 mg, 320 μmol, 1.00 eq, TFA) in DCM (2 mL) was added into the mixture. The resulting mixture was stirred at 20° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely and desired mass was detected. The mixture was concentrated in vacuo. The residue was purified by prep-TLC (SiO₂, EtOAc:MeOH=10:1) to give the title compound (72-2) (100 mg, 137 μmol, 14.3% yield) as a yellow solid. LCMS (ES⁺): m/z 729.2 [M+H]⁺.

Synthesis of 5-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9-oxononyl)oxy)-2-(2,6-dioxopiperidin-3-yl)isoindoline-1,3-dione (Compound 72). To a solution of 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (72-2) (100 mg, 137 μmol, 1.00 eq) in DMF (2 mL) were added 2-(2,6-dioxopiperidin-3-yl)-5-hydroxyisoindoline-1,3-dione (37.6 mg, 137 μmol, 1.00 eq) and K₂CO₃ (56.8 mg, 410 μmol, 3.00 eq) at 20° C. The mixture was stirred at 80° C. for 12 h. LCMS showed that 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (72-2) was consumed completely and the desired mass was detected. The mixture was concentrated in vacuo. The residue was purified by prep-HPLC (column: Phenomenex Luna C18 75*30 mm*3 um; mobile phase: [water (FA)-ACN]; B %: 50%-90%, 8 min) to give the title compound (Compound 72) (2.20 mg, 2.35 μmol, 1.72% yield, 98.7% purity) as a yellow solid. LCMS (ES⁺): m/z 923.3 [M+H]⁺.

### Example 73. Synthesis of 5-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)oxy)-2-(2,6-dioxopiperidin-3-yl)isoindoline-1,3-dione (Compound 73)

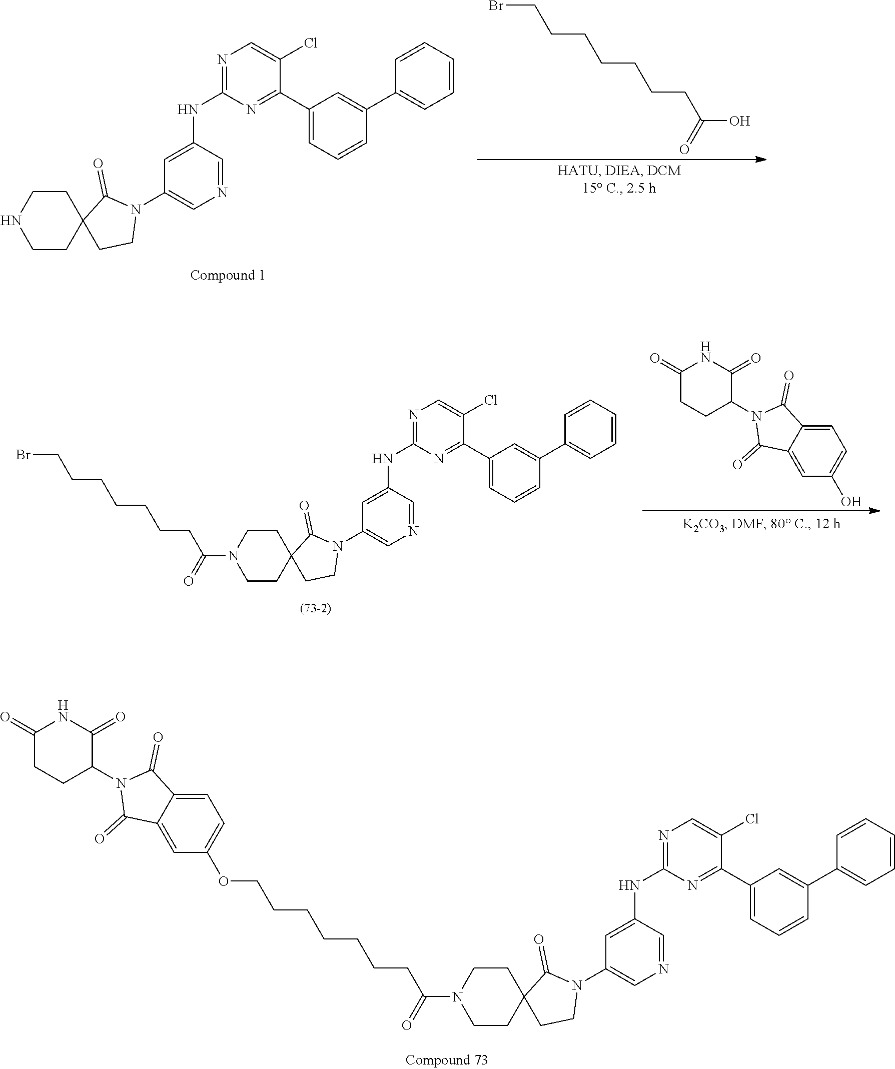 [SMILES: O=C1N(c2cncc(Nc3ncc(Cl)c(-c4cccc(-c5ccccc5)c4)n3)c2)CCC12CCNCC2 ; O=C(O)CCCCCCCBr ; O=C(CCCCCCCBr)N1CCC2(CC1)CCN(c1cncc(Nc3ncc(Cl)c(-c4cccc(-c5ccccc5)c4)n3)c1)C2=O ; O=C1CCC(N2C(=O)c3ccc(O)cc3C2=O)C(=O)N1 ; O=C1CCC(N2C(=O)c3ccc(OCCCCCCCC(=O)N4CCC5(CC4)CCN(c4cncc(Nc6ncc(Cl)c(-c7cccc(-c8ccccc8)c7)n6)c4)C5=O)cc3C2=O)C(=O)N1]

Synthesis of 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (73-2). To a solution of 8-bromooctanoic acid (107 mg, 480 μmol, 1.00 eq) in DCM (5 mL) were added HATU (201 mg, 528 μmol, 1.10 eq) and DIEA (187 mg, 1.44 mmol, 251 μL, 3.00 eq) at 15° C. After addition, the mixture was stirred at this temperature for 0.5 h, and then 2-(5-((4-([1,1′-biphenyl]-3-yl)-5-chloropyrimidin-2-yl)amino)pyridin-3-yl)-2,8-diazaspiro[4.5]decan-1-one (Compound 1) (300 mg, 479.97 μmol, 1.00 eq, TFA) in DCM (2 mL) was added to the mixture at 15° C. The resulting mixture was stirred at 15° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) remained and desired compound was detected. H₂O (3 mL) was added to the solution, then the mixture was extracted with DCM (3 mL*3), the combined organic layers were dried over Na₂SO₄, then concentrated in vacuo. The mixture was purified by prep-TLC (SiO₂, EtOAc:MeOH=10:1) to give the title compound (73-2) (166 mg, 232 μmol, 48.3% yield) as a yellow solid. LCMS (ES⁺): m/z 715.2 [M+H]⁺.

Synthesis of 5-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)oxy)-2-(2,6-dioxopiperidin-3-yl)isoindoline-1,3-dione (Compound 73). To a solution of 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (73-2) (150 mg, 209 μmol, 1.00 eq), 2-(2, 6-dioxopiperidin-3-yl)-5-hydroxyisoindoline-1,3-dione (57.4 mg, 209 μmol, 1.00 eq) in DMF (4 mL) was added K₂CO₃ (86.9 mg, 628 μmol, 3.00 eq) at 15° C. After addition, the mixture was stirred at 80° C. for 12 h. LCMS showed 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (73-2) was consumed completely and desired compound was detected. The mixture was concentrated in vacuo. The residue was purified by prep-HPLC (column: Phenomenex C18 80*40 mm*3 um; mobile phase: [water (NH₄HCO₃)-ACN]; B %: 45%-75%, 8 min) to give the title compound (Compound 73) (10.2 mg, 10.6 μmol, 5.06% yield, 94.5% purity) as white solid. LCMS (ES⁺): m/z 909.3 [M+H]⁺.

### Example 74. Synthesis of 5-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)-7-oxoheptyl)oxy)-2-(2,6-dioxopiperidin-3-yl)isoindoline-1,3-dione (Compound 74)

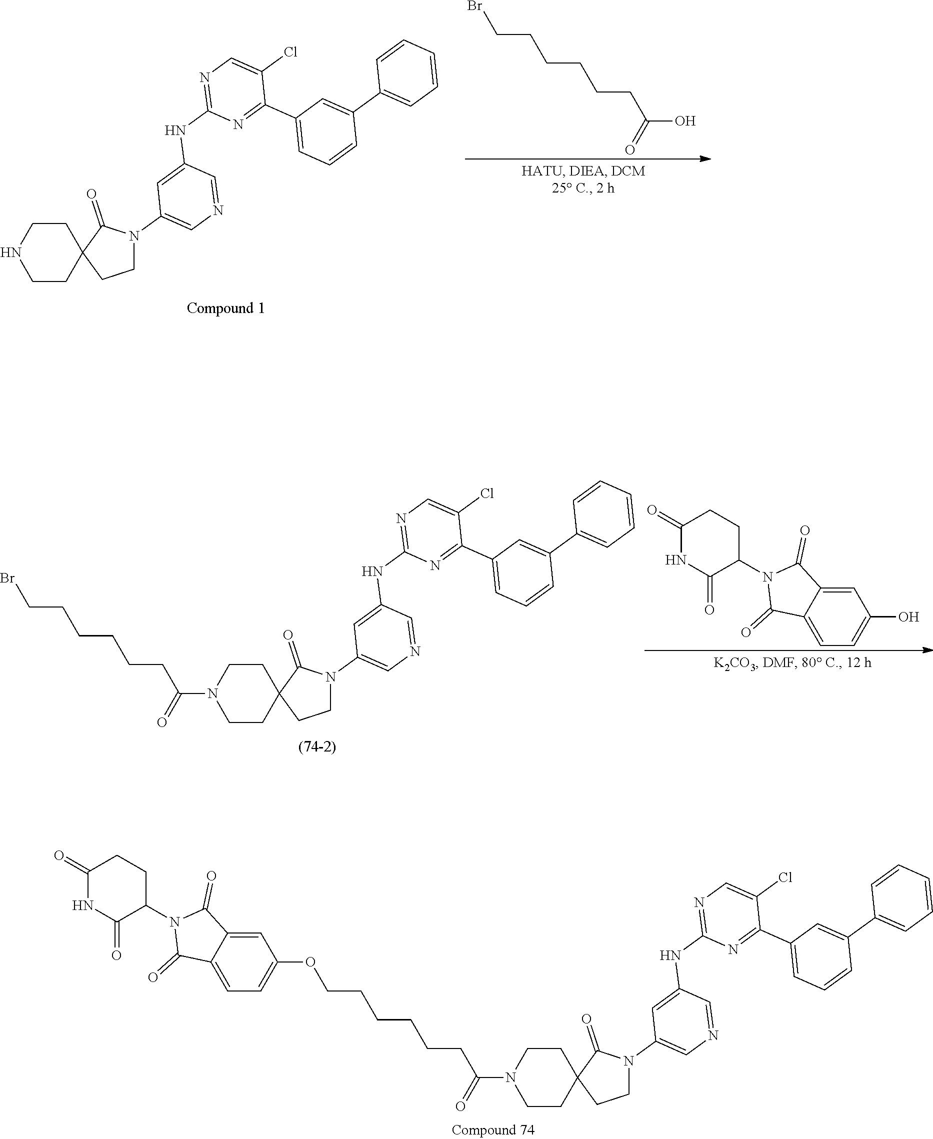

Synthesis of 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (74-2). To a solution of 7-bromoheptanoic acid (100 mg, 480 μmol, 1.00 eq) in DCM (10 mL) were added HATU (201 mg, 528 μmol, 1.10 eq), DIEA (186 mg, 1.44 mmol, 251 μL, 3.00 eq) and 2-(5-((4-([1,1′-biphenyl]-3-yl)-5-chloropyrimidin-2-yl)amino)pyridin-3-yl)-2,8-diazaspiro[4.5]decan-1-one (Compound 1) (300 mg, 480 μmol, 1.00 eq, TFA) at 25° C. The mixture was stirred at 25° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely and the desired mass was detected. The reaction mixture was concentrated under reduced pressure to remove solvent. The residue was purified by prep-TLC (SiO₂, Ethyl acetate:Methanol=10:1) to give the title compound (74-2) (174 mg, 247 μmol, 51.6% yield) as a colorless oil. LCMS (ES⁺): m/z 701.3 [M+H]⁺.

Synthesis of 5-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)-7-oxoheptyl)oxy)-2-(2,6-dioxopiperidin-3-yl)isoindoline-1,3-dione (Compound 74). To a solution of 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (74-2) (174 mg, 247 μmol, 1.00 eq) in DMF (5 mL) were added K₂CO₃ (103 mg, 742 μmol, 3.00 eq) and 2-(2,6-dioxo-3-piperidyl)-5-hydroxy-isoindoline-1,3-dione (67.9 mg, 247 μmol, 1.00 eq) at 25° C. The mixture was stirred at 80° C. for 12 h. LCMS showed 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (74-2) was consumed completely and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 74) (25.9 mg, 28.3 μmol, 11.4% yield, 97.9% purity) as a white solid. LCMS (ES⁺): m/z 895.5 [M+H]⁺.

### Example 75. Synthesis of 5-((6-(2-(5-((4-([1,1′-biphenyl]-3-yl)-5-chloropyrimidin-2-yl)amino)pyridin-3-yl)-1-oxo-2,8-diazaspiro[4.5]decan-8-yl)-6-oxohexyl)oxy)-2-(2,6-dioxopiperidin-3-yl)isoindoline-1,3-dione (Compound 75)

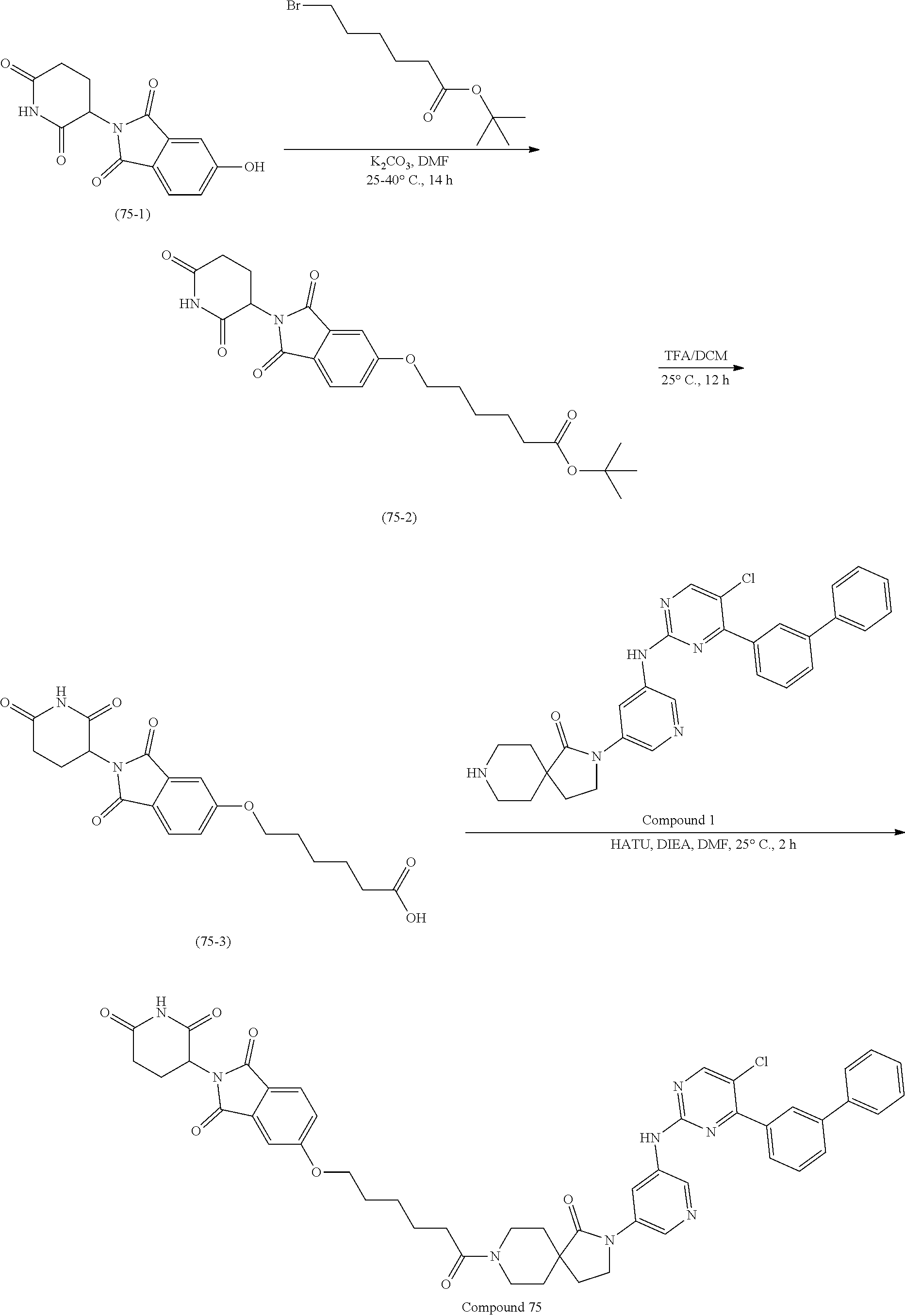 [SMILES: O=C1CCC(N2C(=O)c3ccc(O)cc3C2=O)C(=O)N1 ; CC(C)(C)OC(=O)CCCCCBr ; CC(C)(C)OC(=O)CCCCCOc1ccc2c(c1)C(=O)N(C1CCC(=O)NC1=O)C2=O ; O=C(O)CCCCCOc1ccc2c(c1)C(=O)N(C1CCC(=O)NC1=O)C2=O ; O=C1N(c2cncc(Nc3ncc(Cl)c(-c4cccc(-c5ccccc5)c4)n3)c2)CCC12CCNCC2 ; O=C1CCC(N2C(=O)c3ccc(OCCCCCC(=O)N4CCC5(CC4)CCN(c4cncc(Nc6ncc(Cl)c(-c7cccc(-c8ccccc8)c7)n6)c4)C5=O)cc3C2=O)C(=O)N1]

Synthesis of tert-butyl 6-((2-(2,6-dioxopiperidin-3-yl)-1,3-dioxoisoindolin-5-yl)oxy)hexanoate (75-2). Two batches in parallel were set up. To a stirred solution of 2-(2,6-dioxopiperidin-3-yl)-5-hydroxyisoindoline-1,3-dione (75-1) (250 mg, 912 μmol, 1.00 eq) and tert-butyl 6-bromohexanoate (229 mg, 912 μmol, 1.00 eq) in DMF (6 mL) was added K₂CO₃ (378 mg, 2.73 mmol, 3.00 eq) at 25° C. The resulting mixture was stirred at 25° C. for 12 h. LCMS showed 2-(2,6-dioxopiperidin-3-yl)-5-hydroxyisoindoline-1,3-dione (75-1) remained, desired target MS was detected. The mixture was stirred at 40° C. for 2 h. LCMS showed 2-(2,6-dioxopiperidin-3-yl)-5-hydroxyisoindoline-1,3-dione (75-1) was consumed, desired target MS was detected. Two batches in parallel were combined. The reaction was added to H₂O (8 mL) and then extracted with EtOAc (8 mL*3). The organic layers were combined and washed with brine (8 mL*3), dried over Na₂SO₄, filtered and filtrate was concentrated under reduced pressure to give the crude product. The mixture was purified by prep-TLC (SiO₂, PE:EtOAc=1:1) to give the title compound (75-2) (246 mg, 553 μmol, 30.4% yield) as a yellow oil. LCMS (ES⁺): m/z 445.1 [M+H]⁺.

Synthesis of 6-((2-(2,6-dioxopiperidin-3-yl)-1,3-dioxoisoindolin-5-yl)oxy)hexanoic acid (75-3). To a stirred solution of tert-butyl 6-((2-(2,6-dioxopiperidin-3-yl)-1,3-dioxoisoindolin-5-yl)oxy)hexanoate (75-2) (246 mg, 553 μmol, 1.00 eq) in DCM (3 mL) was added TFA (0.6 mL) at 25° C. The resulting mixture was stirred at 25° C. for 12 h. LCMS showed tert-butyl 6-((2-(2,6-dioxopiperidin-3-yl)-1,3-dioxoisoindolin-5-yl)oxy)hexanoate (75-2) was consumed, desired target MS was detected. The mixture was concentrated in vacuo to give the title compound (75-3) (210 mg, crude) as a colorless oil. LCMS (ES⁺): m/z 389.0 [M+H]⁺.

Synthesis of 5-((6-(2-(5-((4-([1,1′-biphenyl]-3-yl)-5-chloropyrimidin-2-yl)amino)pyridin-3-yl)-1-oxo-2,8-diazaspiro[4.5]decan-8-yl)-6-oxohexyl)oxy)-2-(2,6-dioxopiperidin-3-yl)isoindoline-1,3-dione (Compound 75). To a solution of 6-((2-(2,6-dioxopiperidin-3-yl)-1,3-dioxoisoindolin-5-yl)oxy)hexanoic acid (75-3) (200 mg, 515 μmol, 1.00 eq) in DCM (2 mL) were added HATU (196 mg, 515 μmol, 1.00 eq), HOAt (70.0 mg, 515 μmol, 72.0 μL, 1.00 eq) and DIEA (200 mg, 1.54 mmol, 269 μL, 3.00 eq) at 25° C., then 2-[5-[[5-chloro-4-(3-phenylphenyl)pyrimidin-2-yl]amino]-3-pyridyl]-2,8-diazaspiro[4.5]decan-1-one (Compound 1) (322 mg, 515 μmol, 1.00 eq, TFA) was added to the mixture at 25° C. and the resulting mixture was stirred at 25° C. for 2 h. LCMS showed 6-((2-(2,6-dioxopiperidin-3-yl)-1,3-dioxoisoindolin-5-yl)oxy)hexanoic acid (75-3) was consumed, desired target MS was detected. The organic layer was filtered and the filtrate was concentrated under reduced pressure to give a residue. The residue was purified by prep-HPLC (column: Phenomenex Luna C18 200*40 mm*10 um; mobile phase: [water (FA)-ACN]; B %: 45%-75%, 8 min) to give the title compound (Compound 75) (33.2 mg, 37.3 μmol, 7.24% yield, 98.9% purity) as a white solid. LCMS (ES⁺): m/z 881.2 [M+H]⁺.

### Example 76. Synthesis of 5-((5-(2-(5-((4-([1,1′-biphenyl]-3-yl)-5-chloropyrimidin-2-yl)amino)pyridin-3-yl)-1-oxo-2,8-diazaspiro[4.5]decan-8-yl)-5-oxopentyl)oxy)-2-(2,6-dioxopiperidin-3-yl)isoindoline-1,3-dione (Compound 76)

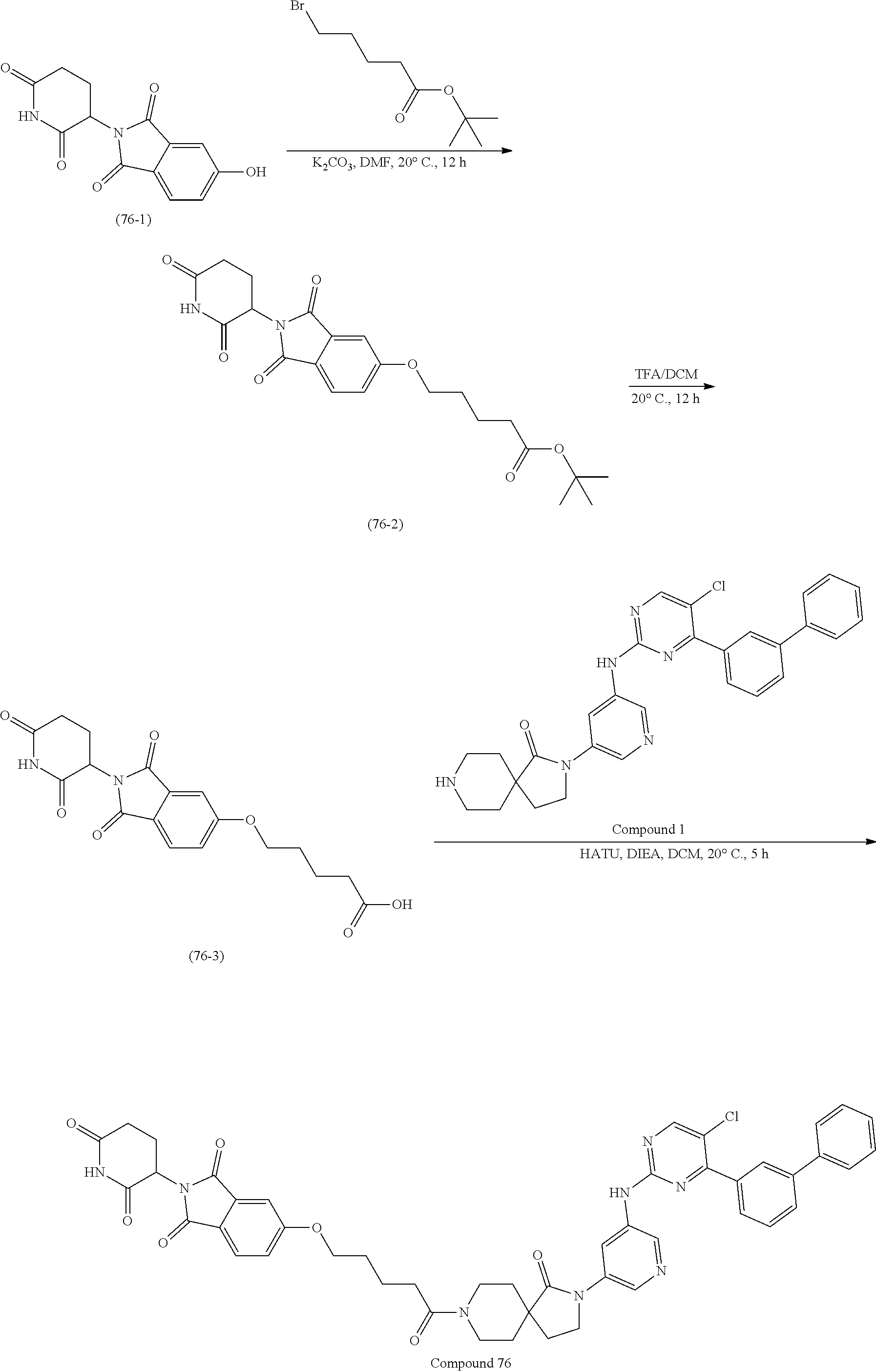 [SMILES: O=C1CCC(N2C(=O)c3ccc(O)cc3C2=O)C(=O)N1 ; CC(C)(C)OC(=O)CCCCBr ; CC(C)(C)OC(=O)CCCCOc1ccc2c(c1)C(=O)N(C1CCC(=O)NC1=O)C2=O ; O=C(O)CCCCOc1ccc2c(c1)C(=O)N(C1CCC(=O)NC1=O)C2=O ; O=C1N(c2cncc(Nc3ncc(Cl)c(-c4cccc(-c5ccccc5)c4)n3)c2)CCC12CCNCC2 ; O=C1CCC(N2C(=O)c3ccc(OCCCCC(=O)N4CCC5(CC4)CCN(c4cncc(Nc6ncc(Cl)c(-c7cccc(-c8ccccc8)c7)n6)c4)C5=O)cc3C2=O)C(=O)N1]

Synthesis of tert-butyl5-((2-(2,6-dioxopiperidin-3-yl)-1,3-dioxoisoindolin-5-yl)oxy)pentanoate (76-2). To a stirred solution of 2-(2,6-dioxopiperidin-3-yl)-5-hydroxyisoindoline-1,3-dione (76-1) (500 mg, 1.82 mmol, 1.00 eq) in DMF (5 mL) were added K₂CO₃ (756 mg, 5.47 mmol, 3.00 eq) and tert-butyl 5-bromopentanoate (432 mg, 1.82 mmol, 1.00 eq) at 20° C. The resulting mixture was stirred at 20° C. for 12 h. LCMS showed 2-(2,6-dioxopiperidin-3-yl)-5-hydroxyisoindoline-1,3-dione (76-1) remained and the desired mass was detected. The mixture was diluted with H₂O (5 mL) and extracted with EtOAc (5 mL*3). The combined organic layers were washed with saturated aqueous NaCl solution (10 mL*3), dried over Na₂SO₄, filtered and the filtrate was concentrated in vacuo. The mixture was purified by prep-TLC (SiO₂, PE:EtOAc=1:1) to give the title compound (76-2) (340 mg, 790 μmol, 43.3% yield) as a colorless oil. LCMS (ES⁺): m/z 431.2 [M+H]⁺.

Synthesis of 5-((2-(2,6-dioxopiperidin-3-yl)-1,3-dioxoisoindolin-5-yl)oxy)pentanoic acid (76-3). A solution of tert-butyl5-((2-(2,6-dioxopiperidin-3-yl)-1,3-dioxoisoindolin-5-yl)oxy)pentanoate (76-2) (340 mg, 790 μmol, 1.00 eq) in TFA (1 mL) and DCM (5 mL) was stirred at 20° C. for 12 h. LCMS showed that tert-butyl5-((2-(2,6-dioxopiperidin-3-yl)-1,3-dioxoisoindolin-5-yl)oxy)pentanoate (76-2) was consumed and the desired mass was detected. The mixture was concentrated in vacuo to give the title compound (76-3) (300 mg, crude) as a yellow oil. LCMS (ES⁺): m/z 375.1 [M+H]⁺.

Synthesis of 5-((5-(2-(5-((4-([1,1′-biphenyl]-3-yl)-5-chloropyrimidin-2-yl)amino)pyridin-3-yl)-1-oxo-2,8-diazaspiro[4.5]decan-8-yl)-5-oxopentyl)oxy)-2-(2,6-dioxopiperidin-3-yl)isoindoline-1,3-dione (Compound 76). To a solution of 5-((2-(2,6-dioxopiperidin-3-yl)-1,3-dioxoisoindolin-5-yl)oxy)pentanoic acid (76-3) (300 mg, 801 μmol, 1.00 eq) in DCM (3 mL) were added HATU (335 mg, 882 μmol, 1.10 eq) and DIEA (311 mg, 2.40 mmol, 419 μL, 3.00 eq) at 20° C., then 2-(5-((4-([1,1′-biphenyl]-3-yl)-5-chloropyrimidin-2-yl)amino)pyridin-3-yl)-2,8-diazaspiro[4.5]decan-1-one (Compound 1) (501 mg, 801 μmol, 1.00 eq, TFA) was added to the mixture at 20° C., and the mixture was stirred at 20° C. for 5 h. LCMS showed 5-((2-(2,6-dioxopiperidin-3-yl)-1,3-dioxoisoindolin-5-yl)oxy)pentanoic acid (76-3) was consumed and the desired mass was detected. The mixture was concentrated in vacuo. The mixture was purified by prep-HPLC (column: Phenomenex Luna C18 200*40 mm*10 um; mobile phase: [water (FA)-ACN]; B %: 35%-75%, 8 min) to give the title compound (Compound 76) (97.2 mg, 110 μmol, 13.8% yield, 98.4% purity) as a white solid. LCMS (ES⁺): m/z 867.3 [M+H]⁺.

### Example 77. Synthesis of N-(8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)-2-((2-(2,6-dioxopiperidin-3-yl)-1,3-dioxoisoindolin-4-yl)oxy)acetamide (Compound 77)

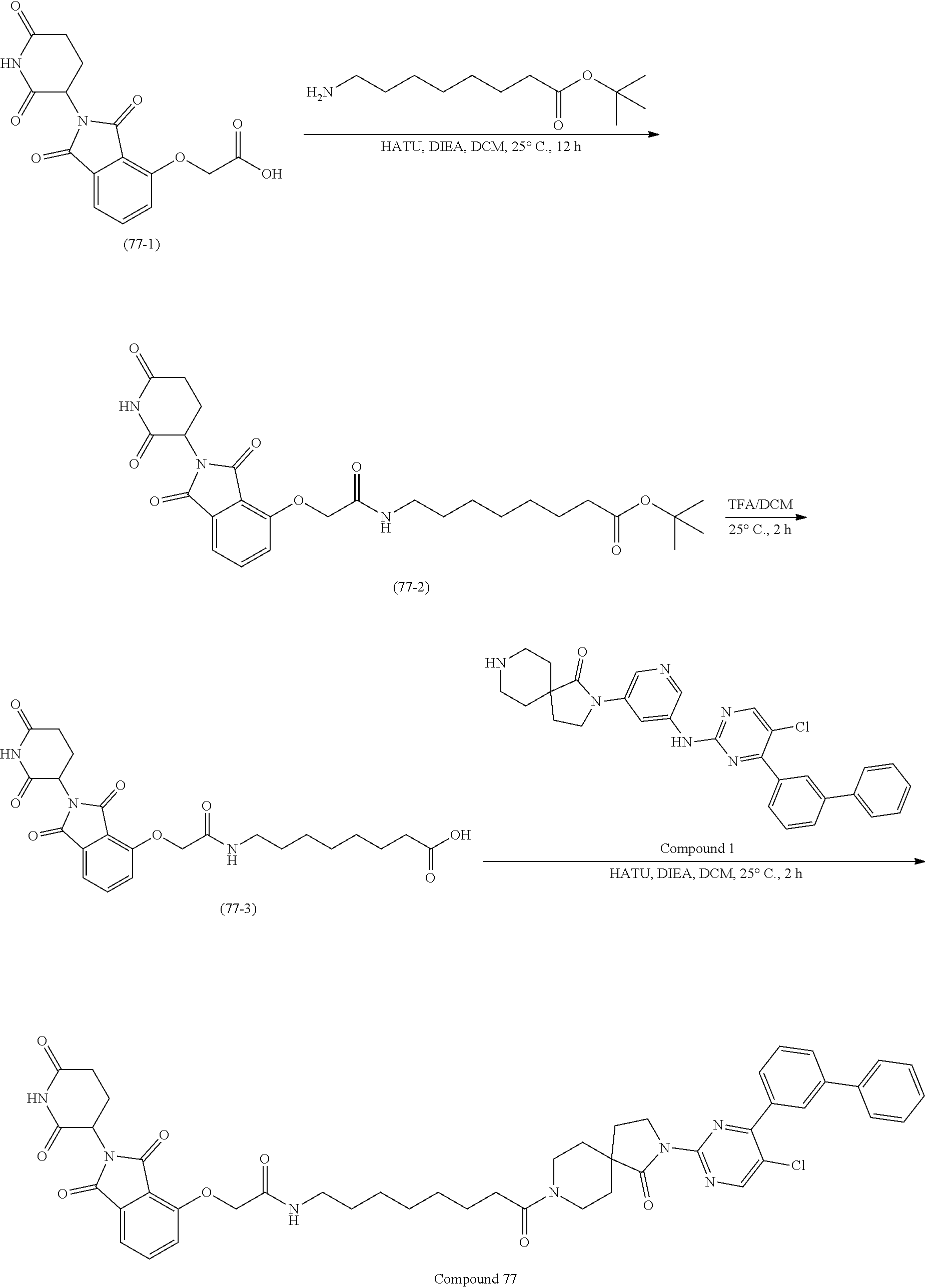 [SMILES: O=C(O)COc1cccc2c1C(=O)N(C1CCC(=O)NC1=O)C2=O ; CC(C)(C)OC(=O)CCCCCCCN ; CC(C)(C)OC(=O)CCCCCCCNC(=O)COc1cccc2c1C(=O)N(C1CCC(=O)NC1=O)C2=O ; O=C(O)CCCCCCCNC(=O)COc1cccc2c1C(=O)N(C1CCC(=O)NC1=O)C2=O ; O=C1N(c2cncc(Nc3ncc(Cl)c(-c4cccc(-c5ccccc5)c4)n3)c2)CCC12CCNCC2 ; O=C(COc1cccc2c1C(=O)N(C1CCC(=O)NC1=O)C2=O)NCCCCCCCC(=O)N1CCC2(CC1)CCN(c1ncc(Cl)c(-c3cccc(-c4ccccc4)c3)n1)C2=O]

Synthesis of tert-butyl 8-(2-((2-(2,6-dioxopiperidin-3-yl)-1,3-dioxoisoindolin-4-yl)oxy)acetamido)octanoate (77-2). To a solution of 2-((2-(2,6-dioxopiperidin-3-yl)-1,3-dioxoisoindolin-4-yl)oxy)acetic acid (77-1) (200 mg, 602 μmol, 1.00 eq) in DCM (5 mL) were added HATU (252 mg, 662 μmol, 1.10 eq), DIEA (233 mg, 1.81 mmol, 315 μL, 3.00 eq) and tert-butyl 8-aminooctanoate (130 mg, 602 μmol, 1.00 eq) at 25° C. The mixture was stirred at 25° C. for 12 h. LCMS showed 2-((2-(2,6-dioxopiperidin-3-yl)-1,3-dioxoisoindolin-4-yl)oxy)acetic acid (77-1) was consumed completely and the desired mass was detected. The reaction mixture was concentrated under reduced pressure to remove solvent. The residue was purified by prep-TLC (SiO₂, Petroleum ether:Ethyl acetate=0:1) to give the title compound (77-2) (135 mg, 254 μmol, 42.3% yield) as a colorless oil. LCMS (ES⁺): m/z 474.2 [M+H−56]⁺.

Synthesis of 8-(2-((2-(2,6-dioxopiperidin-3-yl)-1,3-dioxoisoindolin-4-yl)oxy)acetamido)octanoic acid (77-3). To a solution of tert-butyl 8-(2-((2-(2,6-dioxopiperidin-3-yl)-1,3-dioxoisoindolin-4-yl)oxy)acetamido)octanoate (77-2) (125 mg, 235 μmol, 1.00 eq) in DCM (10 mL) was added TFA (3.08 g, 27.0 mmol, 2.00 mL, 115 eq) at 25° C. The mixture was stirred at 25° C. for 2 h. LCMS showed tert-butyl 8-(2-((2-(2,6-dioxopiperidin-3-yl)-1,3-dioxoisoindolin-4-yl)oxy)acetamido)octanoate (77-2) was consumed completely and the desired mass was detected. The reaction mixture was concentrated under reduced pressure to give the title compound (77-3) (111 mg, crude) as a brown oil. LCMS (ES⁺): m/z 474.2 [M+H]⁺.

Synthesis of N-(8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)-2-((2-(2,6-dioxopiperidin-3-yl)-1,3-dioxoisoindolin-4-yl)oxy)acetamide (Compound 77). To a solution of 8-(2-((2-(2,6-dioxopiperidin-3-yl)-1,3-dioxoisoindolin-4-yl)oxy)acetamido)octanoic acid (77-3) (111 mg, 235 μmol, 1.00 eq) in DCM (5 mL) were added HATU (98.5 mg, 259 μmol, 1.10 eq), DIEA (91.3 mg, 706 μmol, 123 μL, 3.00 eq) and 2-[5-[[5-chloro-4-(3-phenylphenyl)pyrimidin-2-yl]amino]-3-pyridyl]-2,8-diazaspiro[4.5]decan-1-one (Compound 1) (147 mg, 235 μmol, 1.00 eq, TFA) at 25° C. The mixture was stirred at 25° C. for 2 h. LCMS showed 8-(2-((2-(2,6-dioxopiperidin-3-yl)-1,3-dioxoisoindolin-4-yl)oxy)acetamido)octanoic acid (77-3) was consumed completely and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 77) (13.6 mg, 13.7 μmol, 5.81% yield, 97.3% purity) as a white solid. LCMS (ES⁺): m/z 966.5 [M+H]⁺.

### Example 78. Synthesis of 4-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9-oxononyl)oxy)-2-(2,6-dioxopiperidin-3-yl)isoindoline-1,3-dione (Compound 78)

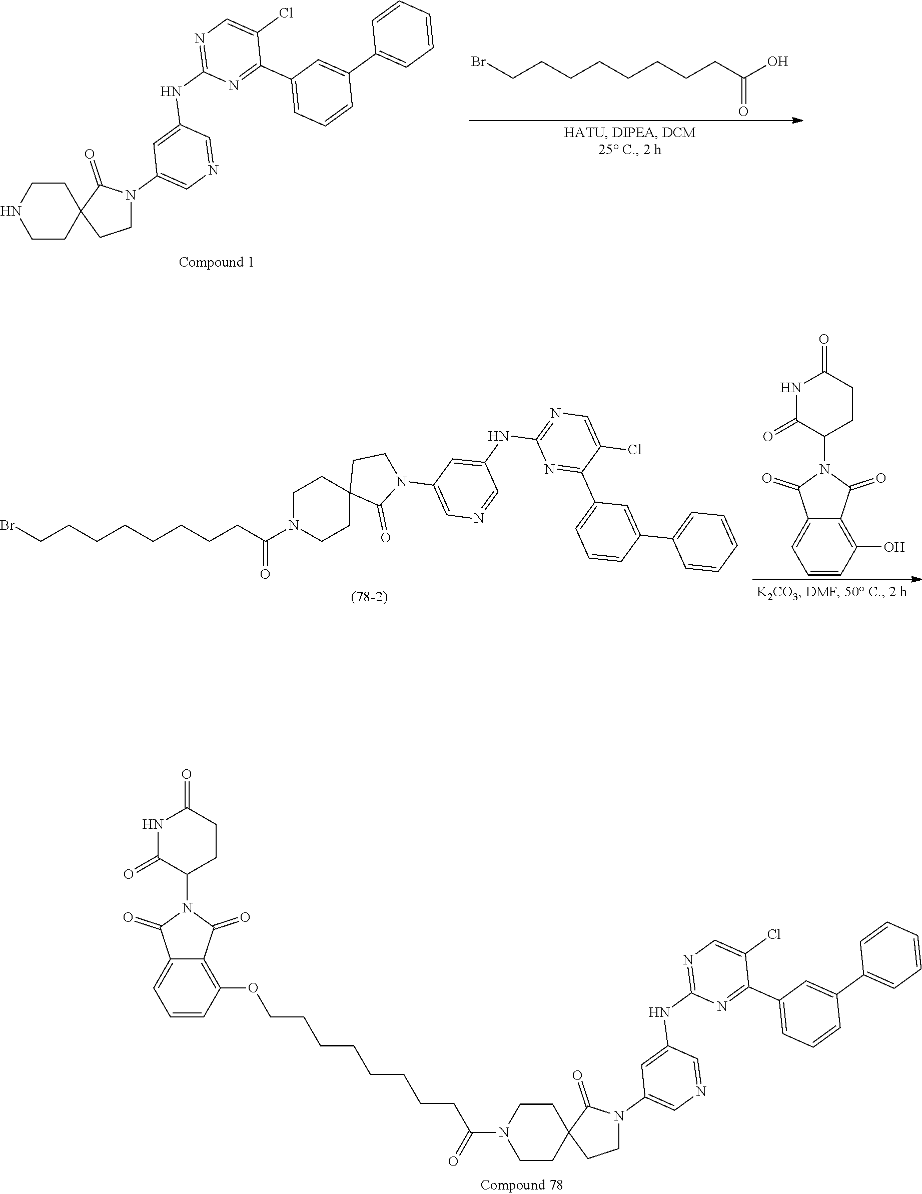 [SMILES: O=C1N(c2cncc(Nc3ncc(Cl)c(-c4cccc(-c5ccccc5)c4)n3)c2)CCC12CCNCC2 ; O=C(O)CCCCCCCCBr ; O=C(CCCCCCCCBr)N1CCC2(CC1)CCN(c1cncc(Nc3ncc(Cl)c(-c4cccc(-c5ccccc5)c4)n3)c1)C2=O ; O=C1CCC(N2C(=O)c3cccc(O)c3C2=O)C(=O)N1 ; O=C1CCC(N2C(=O)c3cccc(OCCCCCCCCC(=O)N4CCC5(CC4)CCN(c4cncc(Nc6ncc(Cl)c(-c7cccc(-c8ccccc8)c7)n6)c4)C5=O)c3C2=O)C(=O)N1]

Synthesis of 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (78-2). To a solution of 9-bromononanoic acid (114 mg, 480 μmol, 1.00 eq) in DCM (10 mL) were added HATU (201 mg, 528 μmol, 1.10 eq), DIEA (186 mg, 1.44 mmol, 251 μL, 3.00 eq) and 2-[5-[[5-chloro-4-(3-phenylphenyl)pyrimidin-2-yl]amino]-3-pyridyl]-2,8-diazaspiro[4.5]decan-1-one (Compound 1) (300 mg, 480 μmol, 1.00 eq, TFA) (78-1) at 25° C. The mixture was stirred at 25° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely and the desired mass was detected. The reaction mixture was concentrated under reduced pressure to remove solvent. The residue was purified by prep-TLC (SiO₂, Ethyl acetate:Methanol=20:1) to give the title compound (78-2) (200 mg, 274 μmol, 57.0% yield) as a pale yellow solid. LCMS (ES⁺): m/z 729.4 [M+H]⁺.

Synthesis of 4-((9-(2-(5-((4-([1,1′-biphenyl]-3-yl)-5-chloropyrimidin-2-yl)amino)pyridin-3-yl)-1-oxo-2,8-diazaspiro[4.5]decan-8-yl)-9-oxononyl)oxy)-2-(2,6-dioxopiperidin-3-yl)isoindoline-1,3-dione (Compound 78). To a solution of 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (78-2) (149 mg, 204 μmol, 1.00 eq) in DMF (10 mL) were added K₂CO₃ (84.6 mg, 612 μmol, 3.00 eq) and 2-(2,6-dioxo-3-piperidyl)-4-hydroxy-isoindoline-1,3-dione (56.0 mg, 204 μmol, 1.00 eq) at 25° C. The mixture was stirred at 50° C. for 2 h. LCMS showed 2-(5-((4-([1,1′-biphenyl]-3-yl)-5-chloropyrimidin-2-yl)amino)pyridin-3-yl)-8-(9-bromononanoyl)-2,8-diazaspiro[4.5]decan-1-one (78-2) was remaining and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 78) (12.7 mg, 13.3 μmol, 6.50% yield, 96.5% purity) as a white solid. LCMS (ES⁺): m/z 923.5 [M+H]⁺.

### Example 79. Synthesis of 4-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)oxy)-2-(2,6-dioxopiperidin-3-yl)isoindoline-1,3-dione (Compound 79)

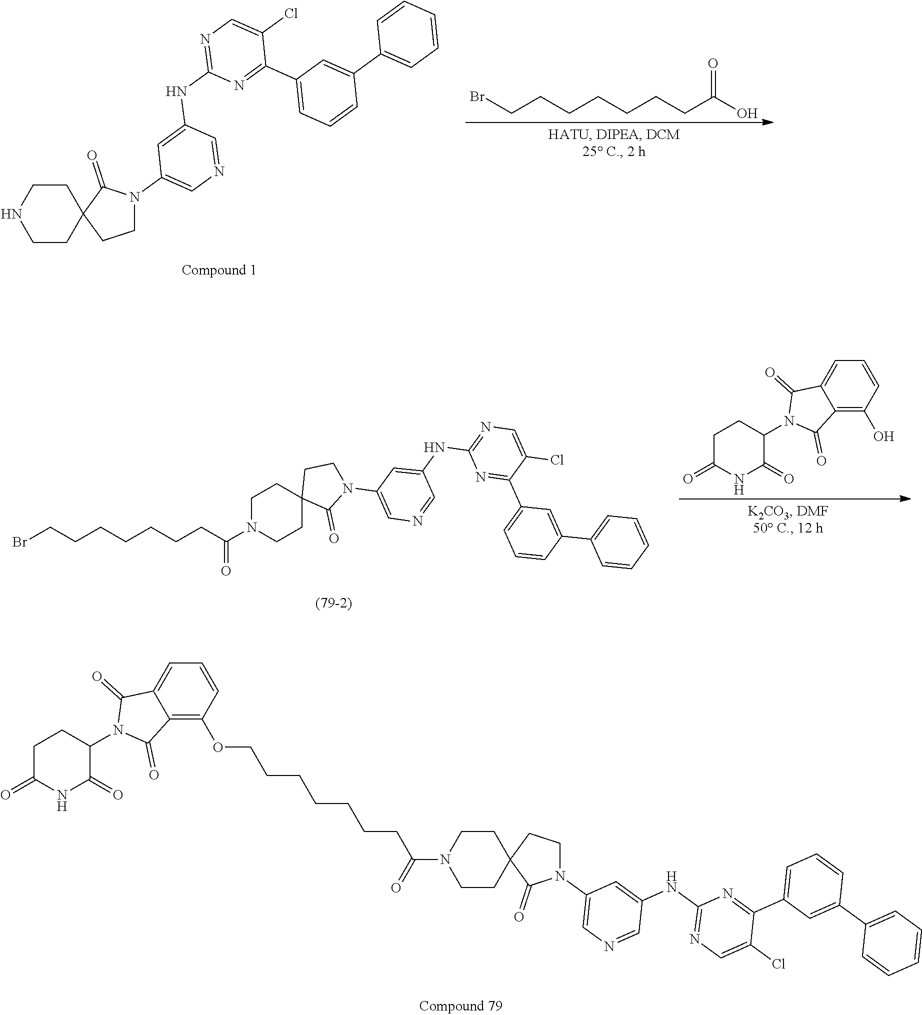 [SMILES: O=C1N(c2cncc(Nc3ncc(Cl)c(-c4cccc(-c5ccccc5)c4)n3)c2)CCC12CCNCC2 ; O=C(O)CCCCCCCBr ; O=C(CCCCCCCBr)N1CCC2(CC1)CCN(c1cncc(Nc3ncc(Cl)c(-c4cccc(-c5ccccc5)c4)n3)c1)C2=O ; O=C1CCC(N2C(=O)c3cccc(O)c3C2=O)C(=O)N1 ; O=C1CCC(N2C(=O)c3cccc(OCCCCCCCC(=O)N4CCC5(CC4)CCN(c4cncc(Nc6ncc(Cl)c(-c7cccc(-c8ccccc8)c7)n6)c4)C5=O)c3C2=O)C(=O)N1]

Synthesis of 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (79-2). To a solution of 8-bromooctanoic acid (107 mg, 480 μmol, 1.00 eq) in DCM (10 mL) were added HATU (201 mg, 528 μmol, 1.10 eq) and DIEA (186 mg, 1.44 mmol, 251 μL, 3.00 eq) at 25° C. Then 2-(5-((4-([1,1′-biphenyl]-3-yl)-5-chloropyrimidin-2-yl)amino)pyridin-3-yl)-2,8-diazaspiro[4.5]decan-1-one (Compound 1) (300 mg, 480 μmol, 1.00 eq, TFA) was added to the mixture at 25° C. The mixture was stirred at 25° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely and the desired mass was detected. The reaction mixture was concentrated under reduced pressure to remove solvent. The residue was purified by prep-TLC (SiO₂, Ethyl acetate:Methanol=20:1) to give the title compound (79-2) (140 mg, 196 μmol, 40.7% yield) as a yellow solid. LCMS (ES⁺): m/z 715.1 [M+H]⁺.

Synthesis of 4-((8-(2-(5-((4-([1,1′-biphenyl]-3-yl)-5-chloropyrimidin-2-yl)amino)pyridin-3-yl)-1-oxo-2,8-diazaspiro[4.5]decan-8-yl)-8-oxooctyl)oxy)-2-(2,6-dioxopiperidin-3-yl)isoindoline-1,3-dione (Compound 79). To a solution of 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (79-2) (130 mg, 181 μmol, 1.00 eq) in DMF (5 mL) were added 2-(2,6-dioxo-3-piperidyl)-4-hydroxy-isoindoline-1,3-dione (49.8 mg, 181 μmol, 1.00 eq), K₂CO₃ (75.3 mg, 545 μmol, 3.00 eq) at 25° C. The mixture was stirred at 50° C. for 12 h. LCMS showed 2-(5-((4-([1,1′-biphenyl]-3-yl)-5-chloropyrimidin-2-yl)amino)pyridin-3-yl)-8-(8-bromooctanoyl)-2,8-diazaspiro[4.5]decan-1-one (79-2) was consumed completely and the desired mass was detected. The reaction mixture was concentrated under reduced pressure to remove solvent. The residue was purified by prep-HPLC (column: Phenomenex C18 80*40 mm*3 um; mobile phase: [water (NH₄HCO₃)-ACN]; B %: 45%-75%, 8 min) to give the title compound (Compound 79) (7.30 mg, 7.94 μmol, 4.37% yield, 98.9% purity) as a white solid. LCMS (ES⁺): m/z 909.3 [M+H]⁺.

### Example 80. Synthesis of 4-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)-7-oxoheptyl)oxy)-2-(2,6-dioxopiperidin-3-yl)isoindoline-1,3-dione (Compound 80)

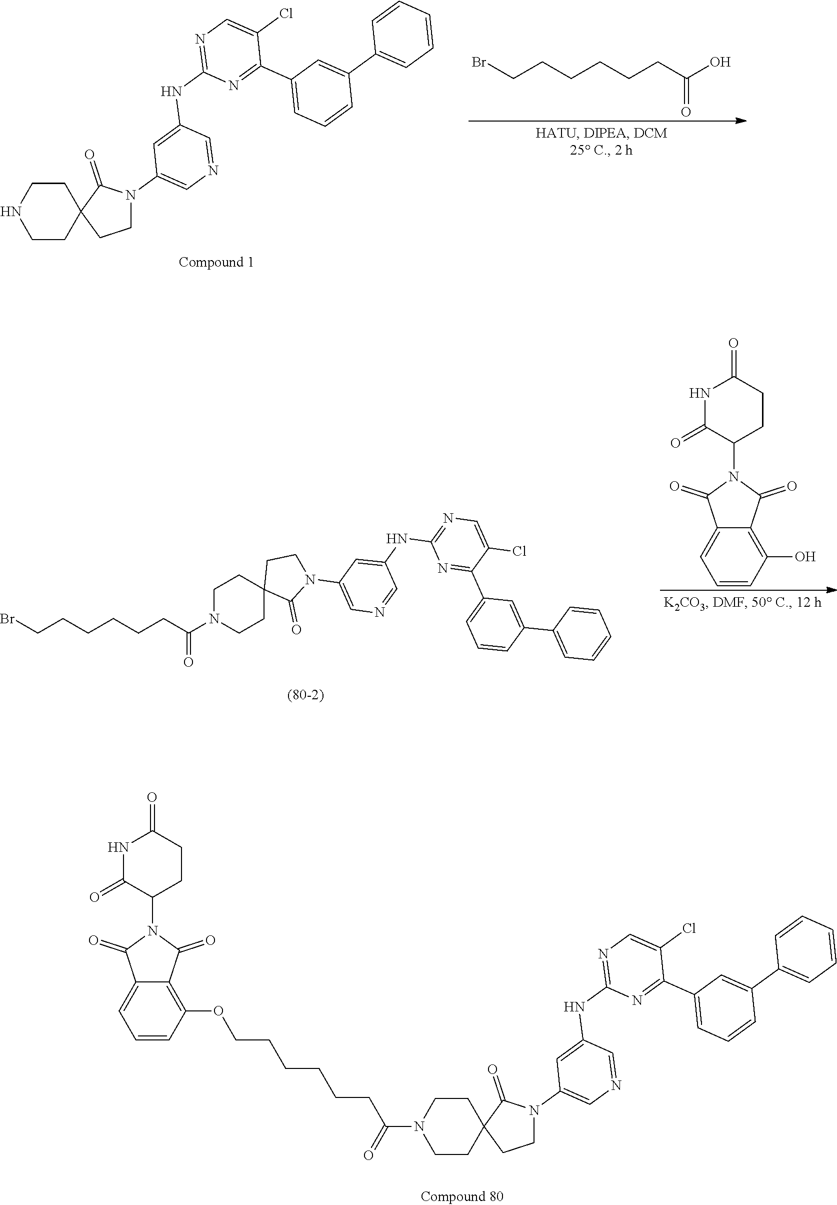 [SMILES: O=C1N(c2cncc(Nc3ncc(Cl)c(-c4cccc(-c5ccccc5)c4)n3)c2)CCC12CCNCC2 ; O=C(O)CCCCCCBr ; O=C(CCCCCCBr)N1CCC2(CC1)CCN(c1cncc(Nc3ncc(Cl)c(-c4cccc(-c5ccccc5)c4)n3)c1)C2=O ; O=C1CCC(N2C(=O)c3cccc(O)c3C2=O)C(=O)N1 ; O=C1CCC(N2C(=O)c3cccc(OCCCCCCC(=O)N4CCC5(CC4)CCN(c4cncc(Nc6ncc(Cl)c(-c7cccc(-c8ccccc8)c7)n6)c4)C5=O)c3C2=O)C(=O)N1]

Synthesis of 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (80-2). To a solution of 7-bromoheptanoic acid (33.5 mg, 160 μmol, 1.00 eq) in DCM (6 mL) were added HATU (66.9 mg, 176 μmol, 1.10 eq), 2-(5-((4-([1,1′-biphenyl]-3-yl)-5-chloropyrimidin-2-yl)amino)pyridin-3-yl)-2,8-diazaspiro[4.5]decan-1-one (Compound 1) (100 mg, 160 μmol, 1.00 eq, TFA) and DIEA (62.0 mg, 480 μmol, 83.6 μL, 3.00 eq) at 25° C. The mixture was stirred at 25° C. for 2 h. LCMS showed 2-(5-((4-([1,1′-biphenyl]-3-yl)-5-chloropyrimidin-2-yl)amino)pyridin-3-yl)-2,8-diazaspiro[4.5]decan-1-one (Compound 1) was consumed completely and the desired mass was detected. The reaction mixture was concentrated under reduced pressure to remove solvent. The residue was purified by prep-TLC (SiO₂, Ethyl acetate:Methanol=20:1) to give the title compound (80-2) (90.0 mg, 103 μmol, 64.1% yield, 80.0% purity) as a yellow solid. LCMS (ES⁺): m/z 701.2 [M+H]⁺.

Synthesis of 4-((7-(2-(5-((4-([1,1′-biphenyl]-3-yl)-5-chloropyrimidin-2-yl)amino)pyridin-3-yl)-1-oxo-2,8-diazaspiro[4.5]decan-8-yl)-7-oxoheptyl)oxy)-2-(2,6-dioxopiperidin-3-yl)isoindoline-1,3-dione (Compound 80). To a solution of 2-(2,6-dioxo-3-piperidyl)-4-hydroxy-isoindoline-1,3-dione (25.0 mg, 91.2 μmol, 1.00 eq) in DMF (3 mL) were added K₂CO₃ (37.8 mg, 273 μmol, 3.00 eq) and 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (80-2) (80.0 mg, 91.2 μmol, 80.0% purity, 1.00 eq) at 25° C. The mixture was stirred at 50° C. for 12 h. LCMS showed 2-(5-((4-([1,1′-biphenyl]-3-yl)-5-chloropyrimidin-2-yl)amino)pyridin-3-yl)-8-(7-bromoheptanoyl)-2,8-diazaspiro[4.5]decan-1-one (80-2) was consumed completely and the desired mass was detected. The organic layers were filtered and the filtrate was concentrated under reduced pressure to give a residue. The residue was purified by prep-HPLC (column: Phenomenex Luna C18 75*30 mm*3 um; mobile phase: [water (FA)-ACN]; B %: 45%-75%, 8 min) to give the title compound (Compound 80) (22.5 mg, 24.5 μmol, 26.9% yield, 97.6% purity) as a white solid. LCMS (ES⁺): m/z 895.3 [M+H]⁺.

### Example 81. Synthesis of N-(5-((4-([1,1′-biphenyl]-3-yl)-5-chloropyrimidin-2-yl)amino)pyridin-3-yl)-8-(4-((2-(2,6-dioxopiperidin-3-yl)-1,3-dioxoisoindolin-4-yl)oxy)butanamido)octanamide (Compound 81)

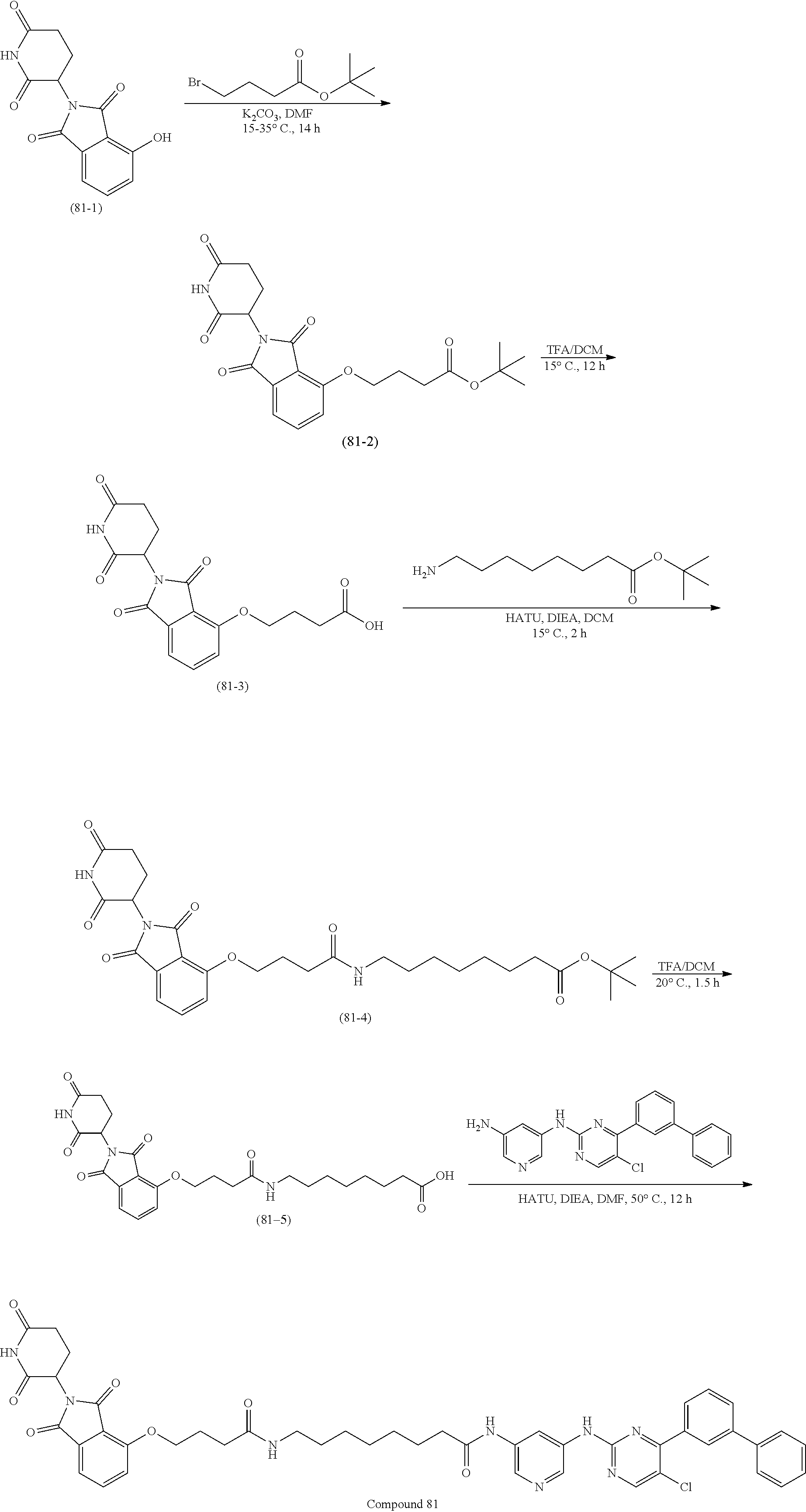

Synthesis of tert-butyl 4-((2-(2,6-dioxopiperidin-3-yl)-1,3-dioxoisoindolin-4-yl)oxy)butanoate (81-2). To a stirred solution of 2-(2,6-dioxopiperidin-3-yl)-4-hydroxyisoindoline-1,3-dione (81-1) (500 mg, 1.82 mmol, 1.00 eq) and tert-butyl 4-bromobutanoate (406 mg, 1.82 mmol, 1.00 eq) in DMF (7 mL) was added K₂CO₃ (755 mg, 5.46 mmol, 3.00 eq) at 15° C. The resulting mixture was stirred at 15° C. for 12 h. LCMS showed that 2-(2,6-dioxopiperidin-3-yl)-4-hydroxyisoindoline-1,3-dione (81-1) remained and the desired mass was detected. The resulting mixture was stirred at 35° C. for 2 h. LCMS showed that the 2-(2,6-dioxopiperidin-3-yl)-4-hydroxyisoindoline-1,3-dione (81-1) remained and the desired mass was detected. The reaction mixture was diluted with water (12 mL) and extracted with EtOAc (3 mL*3). The combined organic layers were washed with brine (2 mL*3), dried with anhydrous Na₂SO₄, filtered and concentrated in vacuo. The mixture was purified by prep-TLC (SiO₂, PE:EtOAc=2:1) to give the title compound (81-2) (240 mg, 576 μmol, 31.7% yield) as a yellow oil. LCMS (ES⁺): m/z 417.1 [M+H]⁺.

Synthesis of 4-((2-(2,6-dioxopiperidin-3-yl)-1,3-dioxoisoindolin-4-yl)oxy)butanoic acid (81-3). A solution of tert-butyl 4-((2-(2,6-dioxopiperidin-3-yl)-1,3-dioxoisoindolin-4-yl)oxy)butanoate (81-2) (240 mg, 576 μmol, 1.00 eq) in TFA (0.4 mL) and DCM (2 mL) was stirred at 15° C. for 12 h. LCMS showed tert-butyl 4-((2-(2,6-dioxopiperidin-3-yl)-1,3-dioxoisoindolin-4-yl)oxy)butanoate (81-2) was consumed, the desired mass was detected. The mixture was concentrate in vacuo to give the title compound (81-3) (200 mg, crude) as a yellow oil. LCMS (ES⁺): m/z 361.1 [M+H]⁺.

Synthesis of tert-butyl 8-(4-((2-(2,6-dioxopiperidin-3-yl)-1,3-dioxoisoindolin-4-yl)oxy)butanamido)octanoate (81-4). To a solution of 4-((2-(2,6-dioxopiperidin-3-yl)-1,3-dioxoisoindolin-4-yl)oxy)butanoic acid (81-3) (200 mg, 555 μmol, 1.00 eq) in DCM (4 mL) were added HATU (232 mg, 611 μmol, 1.10 eq) and DIEA (215 mg, 1.67 mmol, 290 μL, 3.00 eq) at 15° C. Then the tert-butyl 8-aminooctanoate (120 mg, 555 μmol, 1.00 eq) was added to the mixture and stirred at 15° C. for 2 h. LCMS showed that 4-((2-(2,6-dioxopiperidin-3-yl)-1,3-dioxoisoindolin-4-yl)oxy)butanoic acid (81-3) remained and the desired mass was detected. The mixture was concentrated in vacuo. The mixture was purified by prep-TLC (SiO₂, PE:EtOAc=10:1) to give the title compound (81-4) (170 mg, 305 μmol, 54.9% yield) as a white solid. LCMS (ES⁺): m/z 558.2 [M+H]⁺.

Synthesis of 8-(4-((2-(2,6-dioxopiperidin-3-yl)-1,3-dioxoisoindolin-4-yl)oxy)butanamido)octanoic acid (81-5). To a solution of tert-butyl 8-(4-((2-(2,6-dioxopiperidin-3-yl)-1,3-dioxoisoindolin-4-yl)oxy)butanamido)octanoate (81-4) (170 mg, 305 μmol, 1.00 eq) in DCM (3 mL) was added TFA (0.6 mL) at 20° C. The mixture was stirred at 20° C. for 1.5 h. LCMS showed tert-butyl 8-(4-((2-(2,6-dioxopiperidin-3-yl)-1,3-dioxoisoindolin-4-yl)oxy)butanamido)octanoate (81-4) was consumed, desired target MS was detected. The solution was concentrated in vacuo to give the title compound (81-5) (160 mg, crude) as a yellow oil. LCMS (ES⁺): m/z 502.1 [M+H]⁺.

Synthesis of N-(5-((4-([1,1′-biphenyl]-3-yl)-5-chloropyrimidin-2-yl)amino)pyridin-3-yl)-8-(4-((2-(2,6-dioxopiperidin-3-yl)-1,3-dioxoisoindolin-4-yl)oxy)butanamido)octanamide (Compound 81). To a solution of 8-(4-((2-(2,6-dioxopiperidin-3-yl)-1,3-dioxoisoindolin-4-yl)oxy)butanamido)octanoic acid (81-5) (150 mg, 299 μmol, 1.00 eq) in DMF (2 mL) were added HATU (171 mg, 449 μmol, 1.50 eq) and DIEA (232 mg, 1.79 mmol, 313 μL, 6.00 eq) at 20° C., then N³-(4-([1,1′-biphenyl]-3-yl)-5-chloropyrimidin-2-yl)pyridine-3,5-diamine (112 mg, 299 μmol, 1.00 eq) (prepared as described in Example 58) was added to the mixture, the mixture was stirred at 50° C. for 12 h. LCMS showed 8-(4-((2-(2,6-dioxopiperidin-3-yl)-1,3-dioxoisoindolin-4-yl)oxy)butanamido)octanoic acid (81-5) was consumed, desired target MS was detected. The mixture was concentrated in vacuo. The residue was purified by prep-HPLC (column: Phenomenex C18 75*30 mm*3 um; mobile phase: [water (NH₄HCO₃)-ACN]; B %: 45%-65%, 8 min) to give the title compound (Compound 81) (30.2 mg, 33.3 μmol, 11.1% yield, 94.6% purity) as a white solid. LCMS (ES⁺): m/z 857.2 [M+H]⁺.

### Example 82. Synthesis of 3-(5-((6-(2-(5-((4-([1,1′-biphenyl]-3-yl)-5-chloropyrimidin-2-yl)amino)pyridin-3-yl)-1-oxo-2,8-diazaspiro[4.5]decan-8-yl)-6-oxohexyl)amino)-1-oxoisoindolin-2-yl)piperidine-2,6-dione (Compound 82)

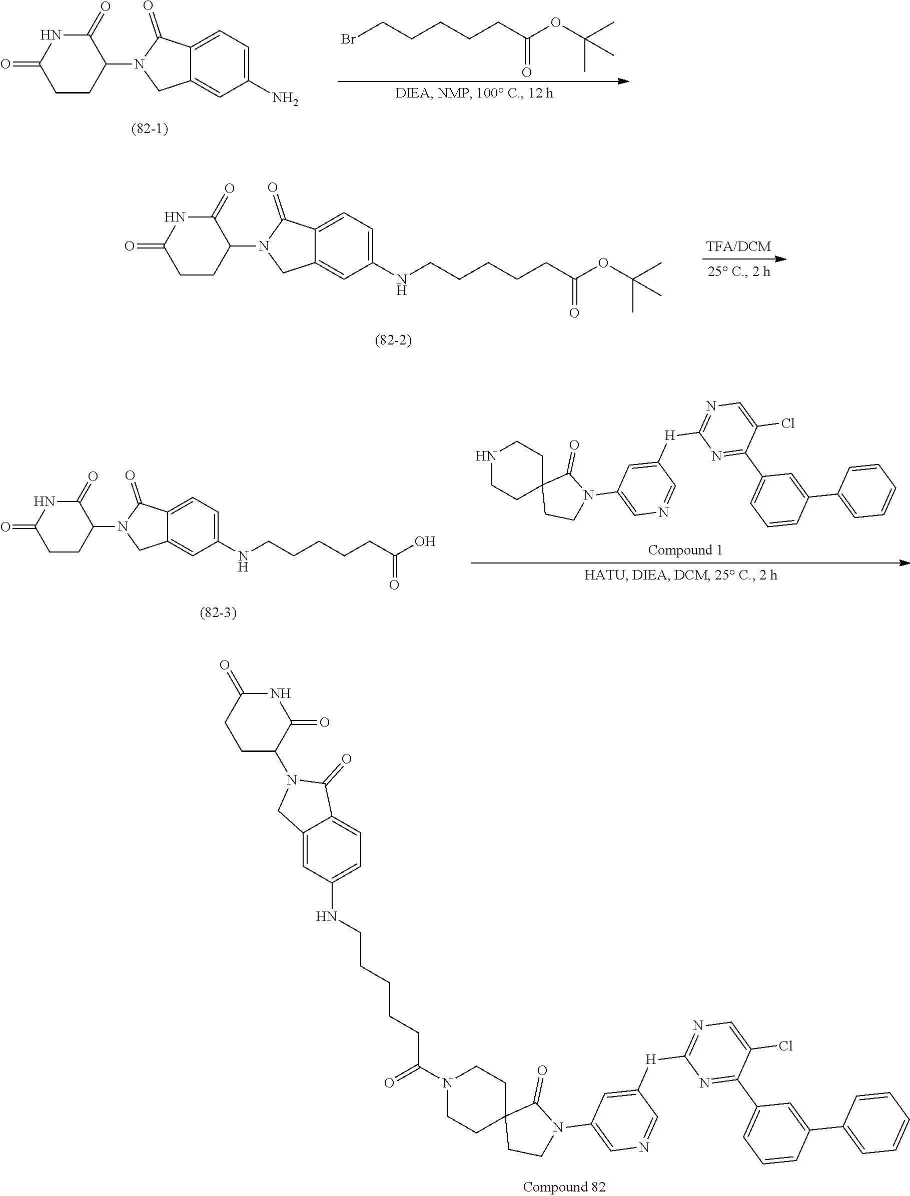

Synthesis of tert-butyl 6-((2-(2,6-dioxopiperidin-3-yl)-1-oxoisoindolin-5-yl)amino)hexanoate (82-2). To a solution of 3-(5-amino-1-oxoisoindolin-2-yl)piperidine-2,6-dione (82-1) (500 mg, 1.93 mmol, 1.00 eq) in NMP (10 mL) were added DIEA (1.25 g, 9.65 mmol, 1.68 mL, 5.00 eq) and tert-butyl 6-bromohexanoate (485 mg, 1.93 mmol, 1.00 eq) at 20° C. The mixture was stirred at 100° C. for 12 h. LCMS showed 3-(5-amino-1-oxoisoindolin-2-yl)piperidine-2,6-dione (82-1) was consumed completely and the desired mass was detected. Water (10 mL) was added to the mixture at 25° C. and extracted with EtOAc (5 mL*3). The combined organic layers were dried over anhydrous sodium sulfate, filtered and the filtrate was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 35%-55%, 8 min) to give the title compound (82-2) (60.0 mg, 140 μmol, 7.24% yield) as a white solid. LCMS (ES⁺): m/z 430.3 [M+H]⁺.

Synthesis of 6-((2-(2,6-dioxopiperidin-3-yl)-1-oxoisoindolin-5-yl)amino)hexanoic acid (82-3). To a solution of tert-butyl 6-((2-(2,6-dioxopiperidin-3-yl)-1-oxoisoindolin-5-yl)amino)hexanoate (82-2) (60.0 mg, 140 μmol, 1.00 eq) in DCM (2.5 mL) was added TFA (0.5 mL) at 25° C. The mixture was stirred at 25° C. for 2 h. LCMS showed tert-butyl 6-((2-(2,6-dioxopiperidin-3-yl)-1-oxoisoindolin-5-yl)amino)hexanoate (82-2) was consumed completely and the desired mass was detected. The reaction mixture was concentrated under reduced pressure to give the title compound (82-3) (52.0 mg, crude) as a brown oil. LCMS (ES⁺): m/z 374.2 [M+H]⁺.

Synthesis of 3-(5-((6-(2-(5-((4-([1,1′-biphenyl]-3-yl)-5-chloropyrimidin-2-yl)amino)pyridin-3-yl)-1-oxo-2,8-diazaspiro[4.5]decan-8-yl)-6-oxohexyl)amino)-1-oxoisoindolin-2-yl)piperidine-2,6-dione (Compound 82). To a solution of 6-((2-(2,6-dioxopiperidin-3-yl)-1-oxoisoindolin-5-yl)amino)hexanoic acid (82-3) (50.0 mg, 134 μmol, 1.00 eq) in DCM (5 mL) were added HATU (50.9 mg, 134 μmol, 1.00 eq) and DIEA (51.9 mg, 402 μmol, 70.0 μL, 3.00 eq) at 25° C. Then 2-[5-[[5-chloro-4-(3-phenylphenyl)pyrimidin-2-yl]amino]-3-pyridyl]-2,8-diazaspiro[4.5]decan-1-one (Compound 1) (83.7 mg, 134 μmol, 1.00 eq, TFA) was added to the mixture at 25° C. The mixture was stirred at 25° C. for 2 h. LCMS showed 6-((2-(2,6-dioxopiperidin-3-yl)-1-oxoisoindolin-5-yl)amino)hexanoic acid (82-3) was consumed completely and the desired mass was detected. The reaction mixture was concentrated under reduced pressure to remove solvent. The residue was purified by prep-HPLC (column: Waters Xbridge BEH C18 100*30 mm*10 um; mobile phase: [water (NH₄HCO₃)-ACN]; B %: 35%-65%, 8 min) to give the title compound (Compound 82) (32.7 mg, 36.0 μmol, 26.9% yield, 95.3% purity) as a white solid. LCMS (ES⁺): m/z 866.3 [M+H]⁺.

### Example 83. Synthesis of N-(5-((4-([1,1′-biphenyl]-3-yl)-5-chloropyrimidin-2-yl)amino)pyridin-3-yl)-6-(6-((2-(2,6-dioxopiperidin-3-yl)-1,3-dioxoisoindolin-4-yl)oxy)hexanamido)hexanamide (Compound 83)

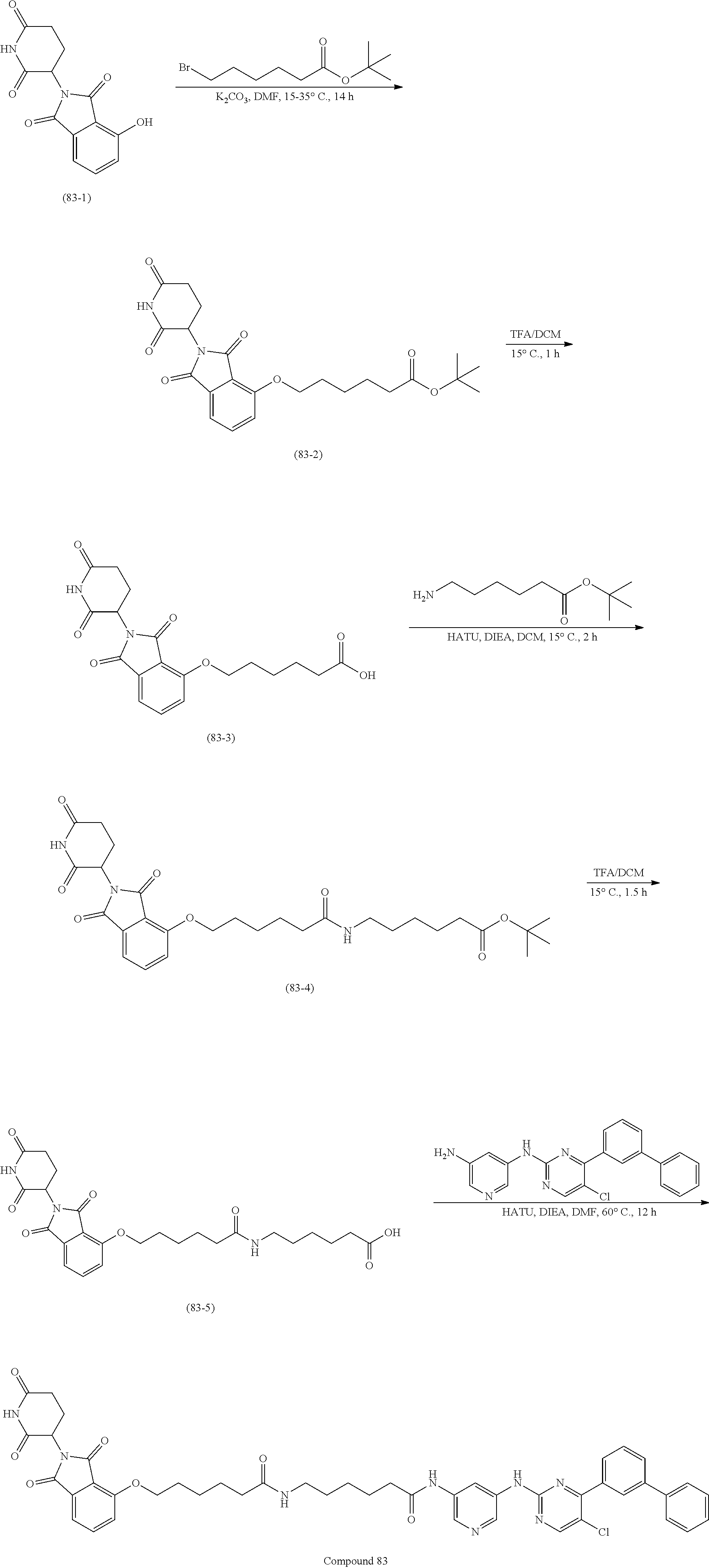

Synthesis of tert-butyl 6-((2-(2,6-dioxopiperidin-3-yl)-1,3-dioxoisoindolin-4-yl)oxy)hexanoate (83-2). To a stirred solution of 2-(2,6-dioxopiperidin-3-yl)-4-hydroxyisoindoline-1,3-dione (83-1) (500 mg, 1.82 mmol, 1.00 eq) and tert-butyl 6-bromohexanoate (458 mg, 1.82 mmol, 1.00 eq) in DMF (7 mL) was added K₂CO₃ (756 mg, 5.47 mmol, 3.00 eq) at 15° C. The resulting mixture was stirred at 15° C. for 12 h. LCMS showed that 2-(2,6-dioxopiperidin-3-yl)-4-hydroxyisoindoline-1,3-dione (83-1) remained and the desired mass was detected. The resulting mixture was stirred at 35° C. for 2 h. LCMS showed that 2-(2,6-dioxopiperidin-3-yl)-4-hydroxyisoindoline-1,3-dione (83-1) was consumed and the desired mass was detected. To the mixture was added H₂O (10 mL) and the mixture was extracted with ethyl acetate (5 mL*3). The combined organic phase were dried with anhydrous Na₂SO₄, filtered and concentrated in vacuo. The mixture was purified by prep-TLC (SiO₂, PE:EtOAc=2:1) to give the title compound (83-2) (260 mg, 585 μmol, 32.1% yield) as a yellow oil. LCMS (ES⁺): m/z 445.2 [M+H]⁺.

Synthesis of 6-((2-(2,6-dioxopiperidin-3-yl)-1,3-dioxoisoindolin-4-yl)oxy)hexanoic acid (83-3). A solution of tert-butyl 6-((2-(2,6-dioxopiperidin-3-yl)-1,3-dioxoisoindolin-4-yl)oxy)hexanoate (83-2) (260 mg, 585 μmol, 1.00 eq) in TFA (0.4 mL) and DCM (2 mL) was stirred at 15° C. for 1 h. LCMS showed that tert-butyl 6-((2-(2,6-dioxopiperidin-3-yl)-1,3-dioxoisoindolin-4-yl)oxy)hexanoate (83-2) was consumed, the desired mass was detected. The mixture was concentrated in vacuo to give the title compound (83-3) (220 mg, crude) as a yellow solid. LCMS (ES⁺): m/z 389.1 [M+H]⁺.

Synthesis of tert-butyl-6-(6-((2-(2,6-dioxopiperidin-3-yl)-1,3-dioxoisoindolin-4-yl)oxy)hexanamido)hexanoate (83-4). To a solution of 6-((2-(2,6-dioxopiperidin-3-yl)-1,3-dioxoisoindolin-4-yl)oxy)hexanoic acid (83-3) (220 mg, 566 μmol, 1.00 eq) in DCM (4 mL) were added HATU (237 mg, 623 μmol, 1.10 eq) and DIEA (172 mg, 1.70 mmol, 240 μL, 3.00 eq) at 15° C. To the mixture was added tert-butyl 6-aminohexanoate (106 mg, 566 μmol, 1.00 eq) and stirred at 15° C. for 2 h. LCMS showed that 6-((2-(2,6-dioxopiperidin-3-yl)-1,3-dioxoisoindolin-4-yl)oxy)hexanoic acid (83-3) was consumed and the desired mass was detected. The mixture was concentrated in vacuo. The mixture was purified by prep-TLC (SiO₂, PE:EtOAc=10:1) to give the title compound (83-4) (130 mg, 233 μmol, 41.2% yield) as a white solid. LCMS (ES⁺): m/z 558.3 [M+H]⁺.

Synthesis of 6-(6-((2-(2,6-dioxopiperidin-3-yl)-1,3-dioxoisoindolin-4-yl)oxy)hexanamido)hexanoic acid (83-5). To a solution of tert-butyl-6-(6-((2-(2,6-dioxopiperidin-3-yl)-1,3-dioxoisoindolin-4-yl)oxy)hexanamido)hexanoate (83-4) (130 mg, 233 μmol, 1.00 eq) in DCM (3 mL) was added TFA (0.60 mL) at 15° C. The mixture was stirred at 15° C. for 1.5 h. LCMS showed tert-butyl-6-(6-((2-(2,6-dioxopiperidin-3-yl)-1,3-dioxoisoindolin-4-yl)oxy)hexanamido)hexanoate (83-4) was consumed, desired target MS was detected. The solution was concentrated in vacuo to give the title compound (83-5) (140 mg, crude) as a yellow oil. LCMS (ES⁺): m/z 502.2 [M+H]⁺.

Synthesis of N-(5-((4-([1,1′-biphenyl]-3-yl)-5-chloropyrimidin-2-yl)amino)pyridin-3-yl)-6-(6-((2-(2,6-dioxopiperidin-3-yl)-1,3-dioxoisoindolin-4-yl)oxy)hexanamido)hexanamide (Compound 83). To a solution of 6-(6-((2-(2,6-dioxopiperidin-3-yl)-1,3-dioxoisoindolin-4-yl)oxy)hexanamido)hexanoic acid (83-5) (130 mg, 259 μmol, 1.00 eq), N³-(4-([1,1′-biphenyl]-3-yl)-5-chloropyrimidin-2-yl)pyridine-3,5-diamine (96.9 mg, 259 μmol, 1.00 eq) (prepared as described in Example 58) in DMF (2 mL) were added HATU (148 mg, 389 μmol, 1.50 eq) and DIEA (201 mg, 1.56 mmol, 271 μL, 6.00 eq) at 15° C. The mixture was stirred at 60° C. for 12 h. LCMS showed 6-(6-((2-(2,6-dioxopiperidin-3-yl)-1,3-dioxoisoindolin-4-yl)oxy)hexanamido)hexanoic acid (83-5) was consumed completely and desired mass was detected. The mixture was concentrated in vacuo. The residue was purified by neutral prep-HPLC (column: Phenomenex C18 75*30 mm*3 um; mobile phase: [water (NH₄HCO₃)-ACN]; B %: 40%-60%, 8 min) to give the title compound (Compound 83) (30.0 mg, 33.7 μmol, 13.0% yield, 96.3% purity) as a yellow oil. LCMS (ES⁺): m/z 857.3 [M+H]⁺.

### Example 84. Synthesis of 3-(5-((5-(2-(5-((4-([1,1′-biphenyl]-3-yl)-5-chloropyrimidin-2-yl)amino)pyridin-3-yl)-1-oxo-2,8-diazaspiro[4.5]decan-8-yl)-5-oxopentyl)amino)-1-oxoisoindolin-2-yl)piperidine-2,6-dione (Compound 84)

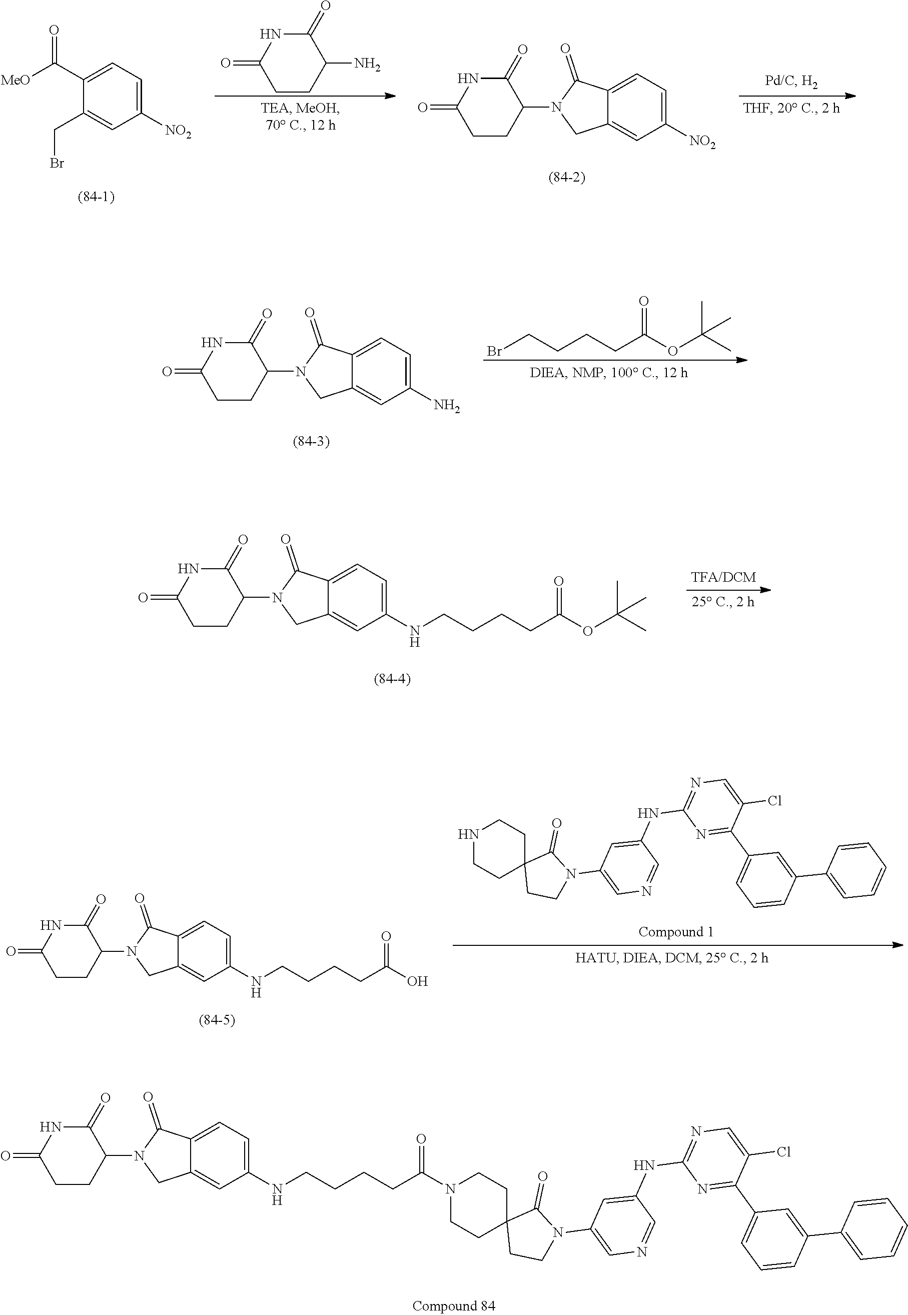

Synthesis of 3-(5-nitro-1-oxoisoindolin-2-yl)piperidine-2,6-dione (84-2). To a solution of methyl 2-(bromomethyl)-4-nitrobenzoate (84-1) (4.00 g, 14.6 mmol, 1.00 eq) in MeOH (50 mL) were added 3-aminopiperidine-2,6-dione; hydrochloride (4.80 g, 29.2 mmol, 2.00 eq) and TEA (3.10 g, 30.7 mmol, 4.27 mL, 2.10 eq) at 20° C. The mixture was stirred at 70° C. for 12 h. LCMS showed the desired mass was detected. TLC (SiO₂, Ethyl acetate:Petroleum ether=3:1) indicated methyl 2-(bromomethyl)-4-nitrobenzoate (84-1) was remaining, and many new spots were detected. The reaction mixture was concentrated under reduced pressure to remove solvent. Water (50 mL) was added to the mixture at 20° C., then the mixture was stirred at 20° C. for 10 min and filtered. Then the filter cake was triturated with PE (50 mL) at 20° C. for 1 h and filtered. The filter cake was dried under reduced pressure to give the title compound (84-2) (4.00 g, 13.8 mmol, 94.8% yield) as a green solid.

Synthesis of 3-(5-amino-1-oxoisoindolin-2-yl)piperidine-2,6-dione (84-3). 10% Pd/C (150 mg, 530 μmol, 50.0% purity) was added to THF (5 mL) at 20° C. under N₂ atmosphere, then 3-(5-nitro-1-oxoisoindolin-2-yl)piperidine-2,6-dione (84-2) (300 mg, 1.04 mmol, 1.00 eq) was added to the mixture at 20° C., the suspension was degassed and purged with H₂ for three times. The mixture was stirred at 20° C. for 2 h under H₂ (15 psi) atmosphere. LCMS showed 3-(5-nitro-1-oxoisoindolin-2-yl)piperidine-2,6-dione (84-2) was consumed completely and the desired mass was detected. The product was filtered and NMP (13 mL) was added at 20° C. Then the filtrate was concentrated under reduced pressure to give the title compound (84-3) (260 mg, crude) as a green liquid in NMP (13 mL). LCMS (ES⁺): m/z 260.1 [M+H]⁺.

Synthesis of tert-butyl 5-((2-(2,6-dioxopiperidin-3-yl)-1-oxoisoindolin-5-yl)amino)pentanoate (84-4). To a solution of 3-(5-amino-1-oxoisoindolin-2-yl)piperidine-2,6-dione (84-3) (260 mg, 1.00 mmol, 1.00 eq) in NMP (13 mL) were added DIEA (648 mg, 5.01 mmol, 873 μL, 5.00 eq) and tert-butyl 5-bromopentanoate (238 mg, 1.00 mmol, 1.00 eq) at 20° C. The mixture was stirred at 100° C. for 12 h. LCMS showed 3-(5-amino-1-oxoisoindolin-2-yl)piperidine-2,6-dione (84-3) was consumed completely and the desired mass was detected. Water (10 mL) was added to the mixture at 20° C. Then the mixture was extracted with EtOAc (5 mL*3). The combined organic layers were dried over anhydrous sodium sulfate, filtered and the filtrate was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 35%-65%, 8 min) to give the title compound (84-4) (50.0 mg, 120 μmol, 12.0% yield) as a white solid. LCMS (ES⁺): m/z 416.2 [M+H]⁺.

Synthesis of 5-((2-(2,6-dioxopiperidin-3-yl)-1-oxoisoindolin-5-yl)amino)pentanoic acid (84-5). To a solution of tert-butyl 5-((2-(2,6-dioxopiperidin-3-yl)-1-oxoisoindolin-5-yl)amino)pentanoate (84-4) (20.0 mg, 48.1 μmol, 1.00 eq) in DCM (2.5 mL) was added TFA (308 mg, 2.70 mmol, 200 μL, 56.1 eq) at 25° C. The mixture was stirred at 25° C. for 2 h. LCMS showed tert-butyl 5-((2-(2,6-dioxopiperidin-3-yl)-1-oxoisoindolin-5-yl)amino)pentanoate (84-4) was consumed completely and the desired mass was detected. The reaction mixture was concentrated under reduced pressure to give the title compound (84-5) (17.0 mg, crude) as a brown oil. LCMS (ES⁺): m/z 360.2 [M+H]⁺.

Synthesis of 3-(5-((5-(2-(5-((4-([1,1′-biphenyl]-3-yl)-5-chloropyrimidin-2-yl)amino)pyridin-3-yl)-1-oxo-2,8-diazaspiro[4.5]decan-8-yl)-5-oxopentyl)amino)-1-oxoisoindolin-2-yl)piperidine-2,6-dione (Compound 84). To a solution of 5-((2-(2,6-dioxopiperidin-3-yl)-1-oxoisoindolin-5-yl)amino)pentanoic acid (84-5) (50.0 mg, 139 μmol, 1.00 eq) in DCM (5 mL) were added HATU (52.9 mg, 139 μmol, 1.00 eq) and DIEA (53.9 mg, 417 μmol, 72.7 μL, 3.00 eq) at 25° C. Then 2-[5-[[5-chloro-4-(3-phenylphenyl)pyrimidin-2-yl]amino]-3-pyridyl]-2,8-diazaspiro[4.5]decan-1-one (Compound 1) (87.0 mg, 139 μmol, 1.00 eq, TFA) was added to the mixture at 25° C. The mixture was stirred at 25° C. for 2 h. LCMS showed 5-((2-(2,6-dioxopiperidin-3-yl)-1-oxoisoindolin-5-yl)amino)pentanoic acid (84-5) was consumed completely and the desired mass was detected. The reaction mixture was concentrated under reduced pressure to remove solvent. The residue was purified by prep-HPLC (column: Phenomenex C18 80*40 mm*3 um; mobile phase: [water (NH₄HCO₃)-ACN]; B %: 40%-60%, 8 min) to give the title compound (Compound 84) (15.5 mg, 17.9 μmol, 12.9% yield, 98.6% purity) as a white solid. LCMS (ES⁺): m/z 852.4 [M+H]⁺.

### Example 85. Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1,3-dioxoisoindolin-4-yl)oxy)acetamido)decanamide (Compound 85)

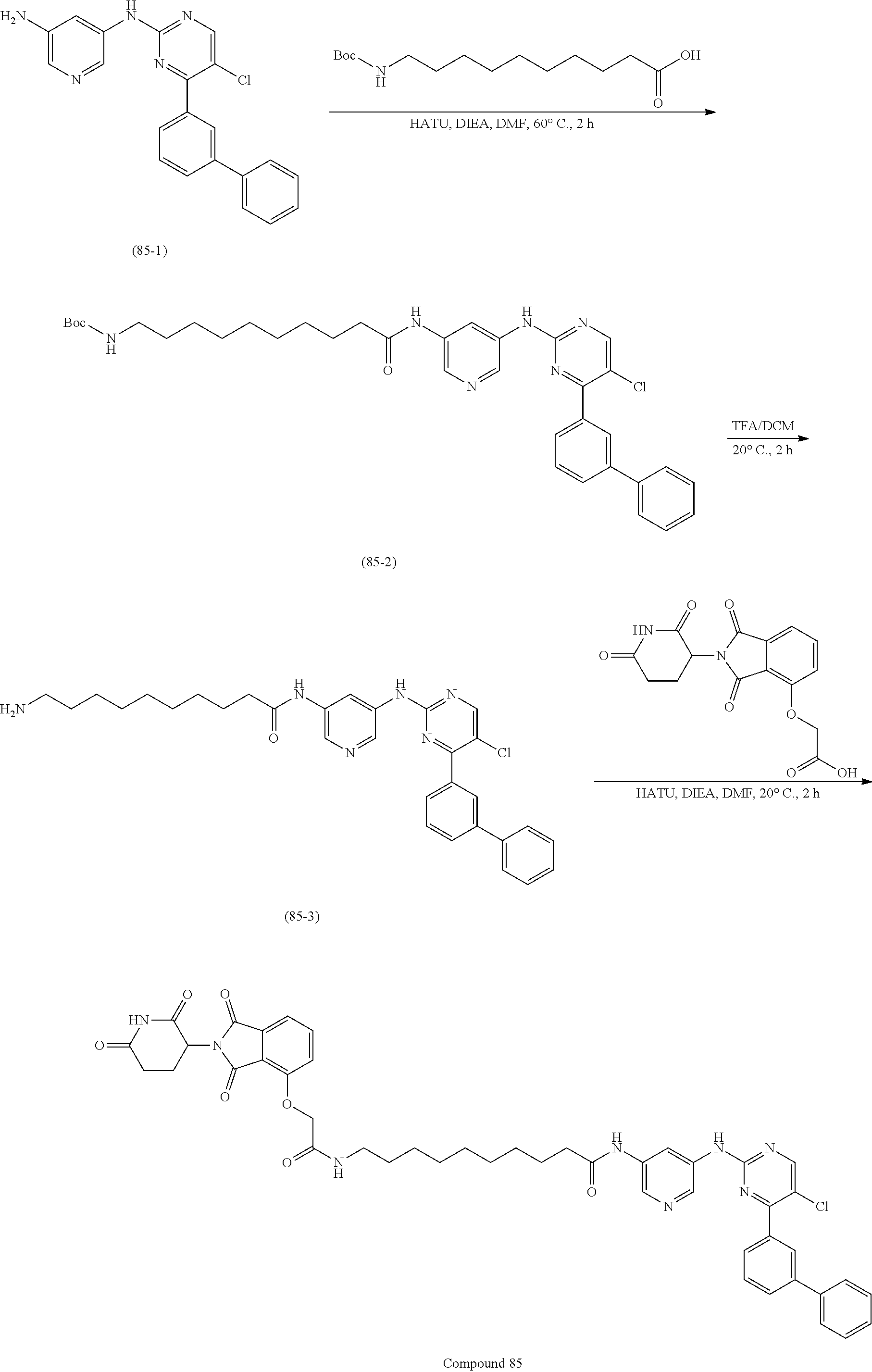 [SMILES: Nc1cncc(Nc2ncc(Cl)c(-c3cccc(-c4ccccc4)c3)n2)c1 ; CC(C)(C)OC(=O)NCCCCCCCCCC(=O)O ; CC(C)(C)OC(=O)NCCCCCCCCCC(=O)Nc1cncc(Nc2ncc(Cl)c(-c3cccc(-c4ccccc4)c3)n2)c1 ; NCCCCCCCCCC(=O)Nc1cncc(Nc2ncc(Cl)c(-c3cccc(-c4ccccc4)c3)n2)c1 ; O=C(O)COc1cccc2c1C(=O)N(C1CCC(=O)NC1=O)C2=O ; O=C(COc1cccc2c1C(=O)N(C1CCC(=O)NC1=O)C2=O)NCCCCCCCCCC(=O)Nc1cncc(Nc2ncc(Cl)c(-c3cccc(-c4ccccc4)c3)n2)c1]

Synthesis of tert-butyl (10-((5-((4-([1,1′-biphenyl]-3-yl)-5-chloropyrimidin-2-yl)amino)pyridin-3-yl)amino)-10-oxodecyl)carbamate (85-2). To a solution of N3-(4-([1,1′-biphenyl]-3-yl)-5-chloropyrimidin-2-yl)pyridine-3,5-diamine (85-1) (prepared as described in Example 58) (300 mg, 802 μmol, 1.00 eq) and 10-(tert-butoxycarbonylamino)decanoic acid (231 mg, 802 μmol, 1.00 eq) in DMF (5 mL) were added HATU (458 mg, 1.20 mmol, 1.50 eq) and DIEA (622 mg, 4.81 mmol, 839 μL, 6.00 eq) at 20° C. The mixture was stirred at 60° C. for 2 h. LCMS showed N3-(4-([1,1′-biphenyl]-3-yl)-5-chloropyrimidin-2-yl)pyridine-3,5-diamine (85-1) was consumed completely and the desired mass was detected. Water (6 mL) was added to the mixture at 20° C. Then the reaction mixture was extracted with EtOAc (4 mL*3). The organic layers were dried over anhydrous sodium sulfate, filtered and the filtrate was concentrated under reduced pressure to give a residue. The residue was purified by prep-TLC (SiO₂, Petroleum ether:Ethyl acetate=0:1) to give the title compound (85-2) (310 mg, 482 μmol, 60.1% yield) as a yellow oil. LCMS (ES⁺): m/z 643.5 [M+H]⁺.

Synthesis of N-(5-((4-([1,1′-biphenyl]-3-yl)-5-chloropyrimidin-2-yl)amino)pyridin-3-yl)-10-aminodecanamide (85-3). To a solution of tert-butyl (10-((5-((4-([1,1′-biphenyl]-3-yl)-5-chloropyrimidin-2-yl)amino)pyridin-3-yl)amino)-10-oxodecyl)carbamate (85-2) (300 mg, 466 μmol, 1.00 eq) in DCM (6 mL) was added TFA (3.08 g, 27.0 mmol, 2.00 mL, 57.9 eq) at 20° C. The mixture was stirred at 20° C. for 2 h. LCMS showed tert-butyl (10-((5-((4-([1,1′-biphenyl]-3-yl)-5-chloropyrimidin-2-yl)amino)pyridin-3-yl)amino)-10-oxodecyl)carbamate (85-2) was consumed completely and the desired mass was detected. The reaction mixture was concentrated under reduced pressure to remove solvent to give the title compound (85-3) (250 mg, crude) as a yellow oil. LCMS (ES⁺): m/z 543.4 [M+H]⁺.

Synthesis of N-(5-((4-([1,1′-biphenyl]-3-yl)-5-chloropyrimidin-2-yl)amino)pyridin-3-yl)-10-(2-((2-(2,6-dioxopiperidin-3-yl)-1,3-dioxoisoindolin-4-yl)oxy)acetamido)decanamide (Compound 85). To a solution of 2-[2-(2,6-dioxo-3-piperidyl)-1,3-dioxo-isoindolin-4-yl]oxyacetic acid (153 mg, 460 μmol, 1.00 eq) in DCM (5 mL) were added HATU (193 mg, 506 μmol, 1.10 eq), N-(5-((4-([1,1′-biphenyl]-3-yl)-5-chloropyrimidin-2-yl)amino)pyridin-3-yl)-10-aminodecanamide (85-3) (250 mg, 460 μmol, 1.00 eq) and DIEA (178 mg, 1.38 mmol, 241 μL, 3.00 eq) at 20° C. The mixture was stirred at 20° C. for 2 h. LCMS showed N-(5-((4-([1,1′-biphenyl]-3-yl)-5-chloropyrimidin-2-yl)amino)pyridin-3-yl)-10-aminodecanamide (85-3) was consumed completely and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH₄HCO₃)-ACN]; B %: 50%-80%, 8 min) to give the crude product. The crude product was purified by prep-HPLC (column: Phenomenex Luna C18 75*30 mm*3 um; mobile phase: [water (FA)-ACN]; B %: 30%-80%, 8 min) to give the title compound (Compound 85) (40.1 mg, 46.8 μmol, 10.2% yield, 100% purity) as a white solid. LCMS (ES⁺): m/z 857.3 [M+H]⁺.

### Example 86. Synthesis of N-(5-((4-([1,1′-biphenyl]-3-yl)-5-chloropyrimidin-2-yl)amino)pyridin-3-yl)-8-(2-((2-(2,6-dioxopiperidin-3-yl)-1,3-dioxoisoindolin-4-yl)oxy)acetamido)octanamide (Compound 86)

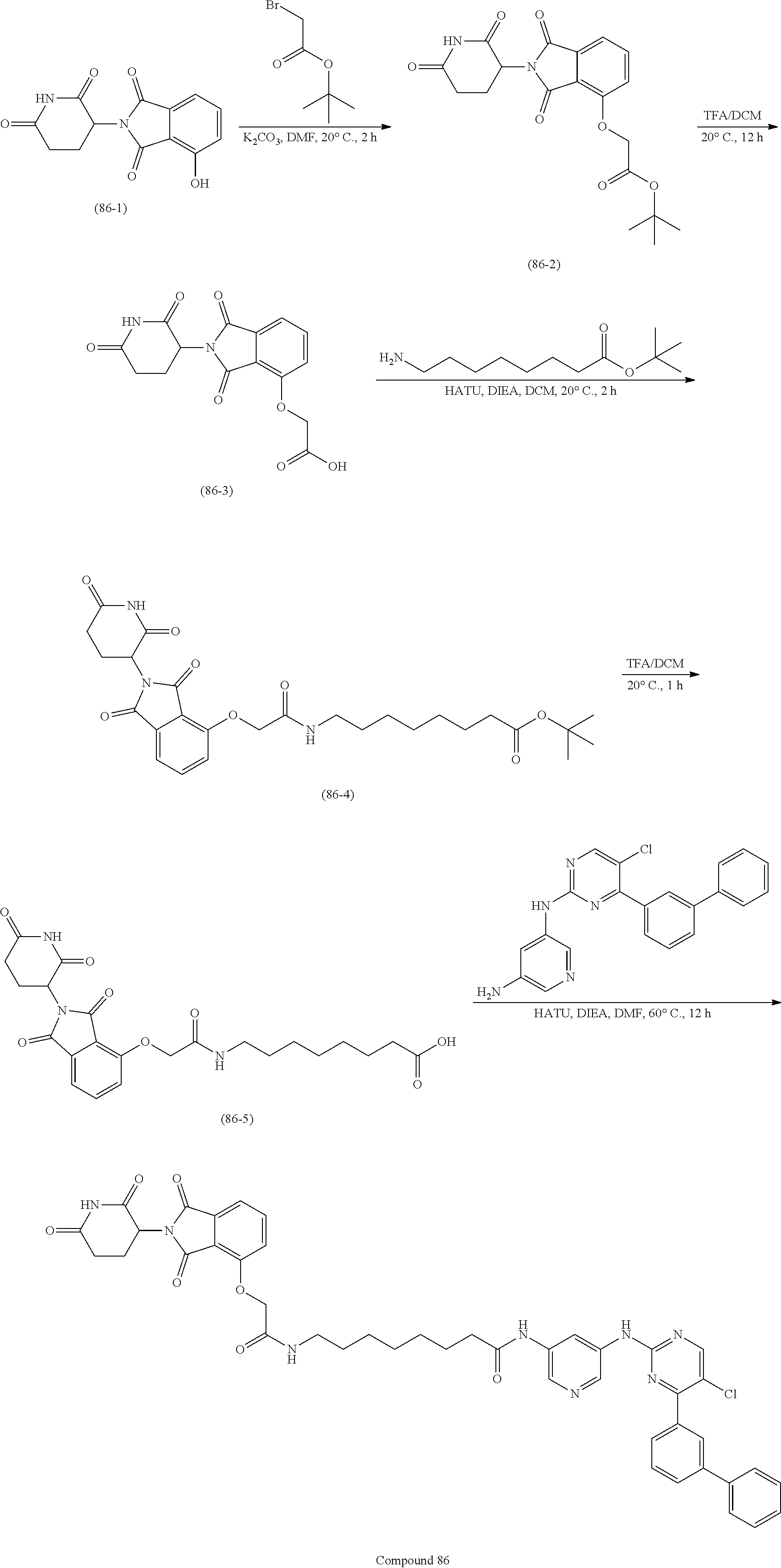

Synthesis of tert-butyl 2-((2-(2,6-dioxopiperidin-3-yl)-1,3-dioxoisoindolin-4-yl)oxy)acetate (86-2). To a solution of 2-(2,6-dioxopiperidin-3-yl)-4-hydroxyisoindoline-1,3-dione (86-1) (4.00 g, 14.6 mmol, 1.00 eq) in DMF (40 mL) were added K₂CO₃ (3.02 g, 21.9 mmol, 1.50 eq) and tert-butyl 2-bromoacetate (2.85 g, 14.6 mmol, 2.16 mL, 1.00 eq) at 20° C. The mixture was stirred at 20° C. for 2 h. LCMS showed ˜12.5% of 2-(2,6-dioxopiperidin-3-yl)-4-hydroxyisoindoline-1,3-dione (86-1) was remaining and desired mass was detected. Water (30 mL) was added to the mixture at 20° C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether:Ethyl acetate=1:0 to 1:3) to give the title compound (86-2) (5.40 g, 13.9 mmol, 95.3% yield) as a white solid. LCMS (ES⁺): m/z 411.1 [M+Na]⁺.

Synthesis of 2-((2-(2,6-dioxopiperidin-3-yl)-1,3-dioxoisoindolin-4-yl)oxy)acetic acid (86-3). To a solution of tert-butyl 2-((2-(2,6-dioxopiperidin-3-yl)-1,3-dioxoisoindolin-4-yl)oxy)acetate (86-2) (5.40 g, 13.9 mmol, 1.00 eq) in DCM (50 mL) was added TFA (15.4 g, 135 mmol, 10.0 mL, 9.71 eq) at 20° C. The mixture was stirred at 20° C. for 12 h. LCMS showed ˜3.3% of tert-butyl 2-((2-(2,6-dioxopiperidin-3-yl)-1,3-dioxoisoindolin-4-yl)oxy)acetate (86-2) was remaining and desired mass was detected. The reaction mixture was concentrated under reduced pressure to remove solvent to give the title compound (86-3) (4.60 g, crude) as a brown oil. LCMS (ES⁺): m/z 333.1 [M+H]⁺.

Synthesis of tert-butyl 8-(2-((2-(2,6-dioxopiperidin-3-yl)-1,3-dioxoisoindolin-4-yl)oxy)acetamido)octanoate (86-4). To a solution of 2-((2-(2,6-dioxopiperidin-3-yl)-1,3-dioxoisoindolin-4-yl)oxy)acetic acid (86-3) (250 mg, 752 μmol, 1.00 eq) in DCM (5 mL) were added HATU (315 mg, 828 μmol, 1.10 eq), DIEA (292 mg, 2.26 mmol, 393 μL, 3.00 eq) and tert-butyl 8-aminooctanoate (162 mg, 752 μmol, 1.00 eq) at 20° C. The mixture was stirred at 20° C. for 2 h. LCMS showed ˜12.1% of 2-((2-(2,6-dioxopiperidin-3-yl)-1,3-dioxoisoindolin-4-yl)oxy)acetic acid (86-3) was remaining and desired mass was detected. The reaction mixture was concentrated under reduced pressure to remove solvent. The residue was purified by prep-TLC (SiO₂, Ethyl acetate:Petroleum ether=3:1) to give the title compound (86-4) (200 mg, 378 μmol, 50.2% yield) as a colorless oil. LCMS (ES⁺): m/z 474.2 [M+H−56]⁺

Synthesis of 8-(2-((2-(2,6-dioxopiperidin-3-yl)-1,3-dioxoisoindolin-4-yl)oxy)acetamido)octanoic acid (86-5). To a solution of tert-butyl 8-(2-((2-(2,6-dioxopiperidin-3-yl)-1,3-dioxoisoindolin-4-yl)oxy)acetamido)octanoate (86-4) (200 mg, 378 μmol, 1.00 eq) in DCM (4 mL) was added TFA (1.23 g, 10.8 mmol, 0.800 mL, 28.6 eq) at 20° C. The mixture was stirred at 20° C. for 1 h. LCMS showed tert-butyl 8-(2-((2-(2,6-dioxopiperidin-3-yl)-1,3-dioxoisoindolin-4-yl)oxy)acetamido)octanoate (86-4) was consumed completely and the desired mass was detected. The reaction mixture was concentrated under reduced pressure to remove solvent to give the title compound (86-5) (178 mg, crude) as a yellow oil. LCMS (ES⁺): m/z 474.2 [M+H]⁺.

Synthesis of N-(5-((4-([1,1′-biphenyl]-3-yl)-5-chloropyrimidin-2-yl)amino)pyridin-3-yl)-8-(2-((2-(2,6-dioxopiperidin-3-yl)-1,3-dioxoisoindolin-4-yl)oxy)acetamido)octanamide (Compound 86). To a solution of 8-(2-((2-(2,6-dioxopiperidin-3-yl)-1,3-dioxoisoindolin-4-yl)oxy)acetamido)octanoic acid (86-5) (165 mg, 348 μmol, 1.00 eq) in DMF (4 mL) were added HATU (199 mg, 523 μmol, 1.50 eq), DIEA (270 mg, 2.09 mmol, 364 μL, 6.00 eq) and N3-[5-chloro-4-(3-phenylphenyl)pyrimidin-2-yl]pyridine-3,5-diamine (130 mg, 348 μmol, 1.00 eq) (prepared as described in Example 58) at 20° C. The mixture was stirred at 60° C. for 12 h. LCMS showed ˜1.5% of 8-(2-((2-(2,6-dioxopiperidin-3-yl)-1,3-dioxoisoindolin-4-yl)oxy)acetamido)octanoic acid (86-5) was remaining and desired mass was detected. The reaction mixture was concentrated under reduced pressure to give a residue. The residue was purified by prep-HPLC (column: Waters Xbridge 150*25 mm*5 um; mobile phase: [water (NH₄HCO₃)-ACN]; B %: 50%-80%, 8 min) to give the title compound (Compound 86) (60.2 mg, 69.8 μmol, 20.0% yield, 96.2% purity) as a pink solid. LCMS (ES⁺): m/z 829.2 [M+H]⁺.

### Example 87. Synthesis of 3-(5-((5-(2-(5-((4-([1,1′-biphenyl]-3-yl)-5-chloropyrimidin-2-yl)amino)pyridin-3-yl)-1-oxo-2,8-diazaspiro[4.5]decan-8-yl)-5-oxopentyl)oxy)-1-oxoisoindolin-2-yl)piperidine-2,6-dione (Compound 87)

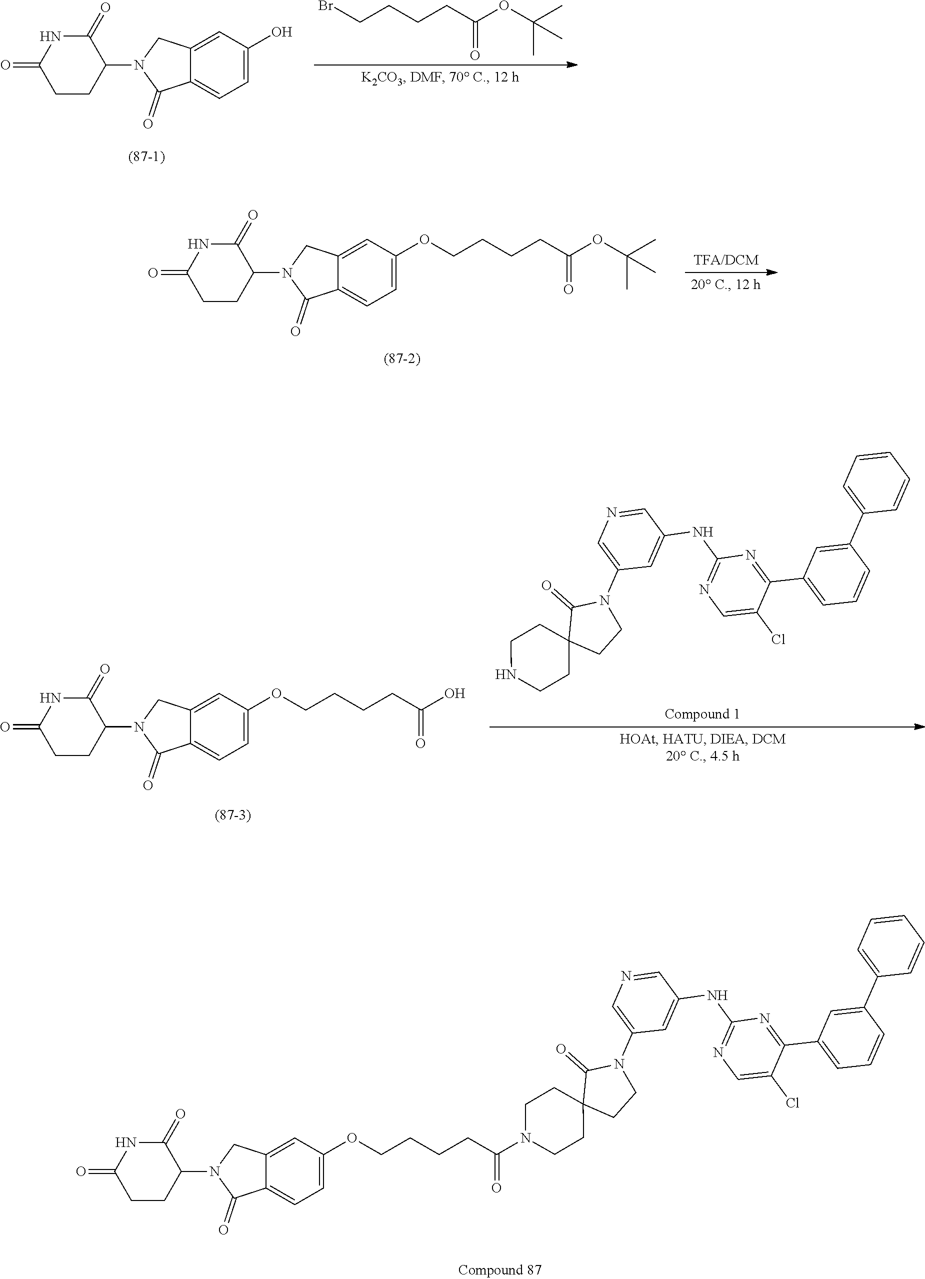 [SMILES: O=C1CCC(N2Cc3cc(O)ccc3C2=O)C(=O)N1 ; CC(C)(C)OC(=O)CCCCBr ; CC(C)(C)OC(=O)CCCCOc1ccc2c(c1)CN(C1CCC(=O)NC1=O)C2=O ; O=C(O)CCCCOc1ccc2c(c1)CN(C1CCC(=O)NC1=O)C2=O ; O=C1N(c2cncc(Nc3ncc(Cl)c(-c4cccc(-c5ccccc5)c4)n3)c2)CCC12CCNCC2 ; O=C1CCC(N2Cc3cc(OCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1]

Synthesis of tert-butyl 5-((2-(2,6-dioxopiperidin-3-yl)-1-oxoisoindolin-5-yl)oxy)pentanoate (87-2). To a solution of tert-butyl 5-bromopentanoate (364 mg, 1.54 mmol, 0.800 eq) in DMF (5 mL) were added K₂CO₃ (797 mg, 5.76 mmol, 3.00 eq) and 3-(5-hydroxy-1-oxoisoindolin-2-yl)piperidine-2,6-dione (87-1) (500 mg, 1.92 mmol, 1.00 eq) at 20° C. The mixture was stirred at 70° C. for 12 h. LCMS showed 3-(5-hydroxy-1-oxoisoindolin-2-yl)piperidine-2,6-dione (87-1) was consumed completely and the desired mass was detected. The reaction mixture was filtered and the filtrate was concentrated under reduced pressure to give a residue. The residue was purified by prep-HPLC (column: Phenomenex Luna C18 75*30 mm*3 um; mobile phase: [water (FA)-ACN]; B %: 5%-45%, 8 min) to give the title compound (87-2) (126 mg, 303 μmol, 15.8% yield) as a white solid. LCMS (ES⁺): m/z 439.3 [M+Na]⁺.

Synthesis of 5-((2-(2,6-dioxopiperidin-3-yl)-1-oxoisoindolin-5-yl)oxy)pentanoic acid (87-3). To a solution of tert-butyl 5-((2-(2,6-dioxopiperidin-3-yl)-1-oxoisoindolin-5-yl)oxy)pentanoate (87-2) (110 mg, 264 μmol, 1.00 eq) in DCM (4 mL) was added TFA (1.23 g, 10.8 mmol, 0.80 mL, 40.9 eq) at 20° C. The mixture was stirred at 20° C. for 12 h. LCMS showed tert-butyl 5-((2-(2,6-dioxopiperidin-3-yl)-1-oxoisoindolin-5-yl)oxy)pentanoate (87-2) was consumed completely and the desired mass was detected. The reaction mixture was concentrated under reduced pressure to remove solvent to give the title compound (87-3) (95.0 mg, crude) as a yellow oil. LCMS (ES⁺): m/z 361.2 [M+H]⁺.

Synthesis of 3-(5-((5-(2-(5-((4-([1,1′-biphenyl]-3-yl)-5-chloropyrimidin-2-yl)amino)pyridin-3-yl)-1-oxo-2,8-diazaspiro[4.5]decan-8-yl)-5-oxopentyl)oxy)-1-oxoisoindolin-2-yl)piperidine-2,6-dione (Compound 87). To a solution of 5-((2-(2,6-dioxopiperidin-3-yl)-1-oxoisoindolin-5-yl)oxy)pentanoic acid (87-3) (95.0 mg, 264 μmol, 1.00 eq) in DCM (6 mL) were added HATU (100 mg, 264 μmol, 1.00 eq), HOAt (35.9 mg, 264 μmol, 36.9 μL, 1.00 eq) and DIEA (102 mg, 791 μmol, 138 μL, 3.00 eq) at 20° C. The mixture was stirred at 20° C. for 0.5 h. 2-[5-[[5-chloro-4-(3-phenylphenyl)pyrimidin-2-yl]amino]-3-pyridyl]-2,8-diazaspiro[4.5]decan-1-one (Compound 1) (165 mg, 264 μmol, 1.00 eq, TFA) in DCM (2 mL) added to the mixture at 20° C. The mixture was stirred at 20° C. for 4 h. LCMS showed 5-((2-(2,6-dioxopiperidin-3-yl)-1-oxoisoindolin-5-yl)oxy)pentanoic acid (87-3) was consumed completely and the desired mass was detected. The reaction mixture was concentrated under reduced pressure to remove solvent. The residue was purified by prep-HPLC (column: Phenomenex Luna 80*30 mm*3 um; mobile phase: [water (FA)-ACN]; B %: 30%-60%, 8 min) to give the title compound (Compound 87) (88.2 mg, 101 μmol, 38.2% yield, 97.5% purity) as a pink solid. LCMS (ES⁺): m/z 853.3 [M+H]⁺.

### Example 88. Synthesis of 3-(5-((6-(2-(5-((4-([1,1′-biphenyl]-3-yl)-5-chloropyrimidin-2-yl)amino)pyridin-3-yl)-1-oxo-2,8-diazaspiro[4.5]decan-8-yl)-6-oxohexyl)oxy)-1-oxoisoindolin-2-yl)piperidine-2,6-dione (Compound 88)

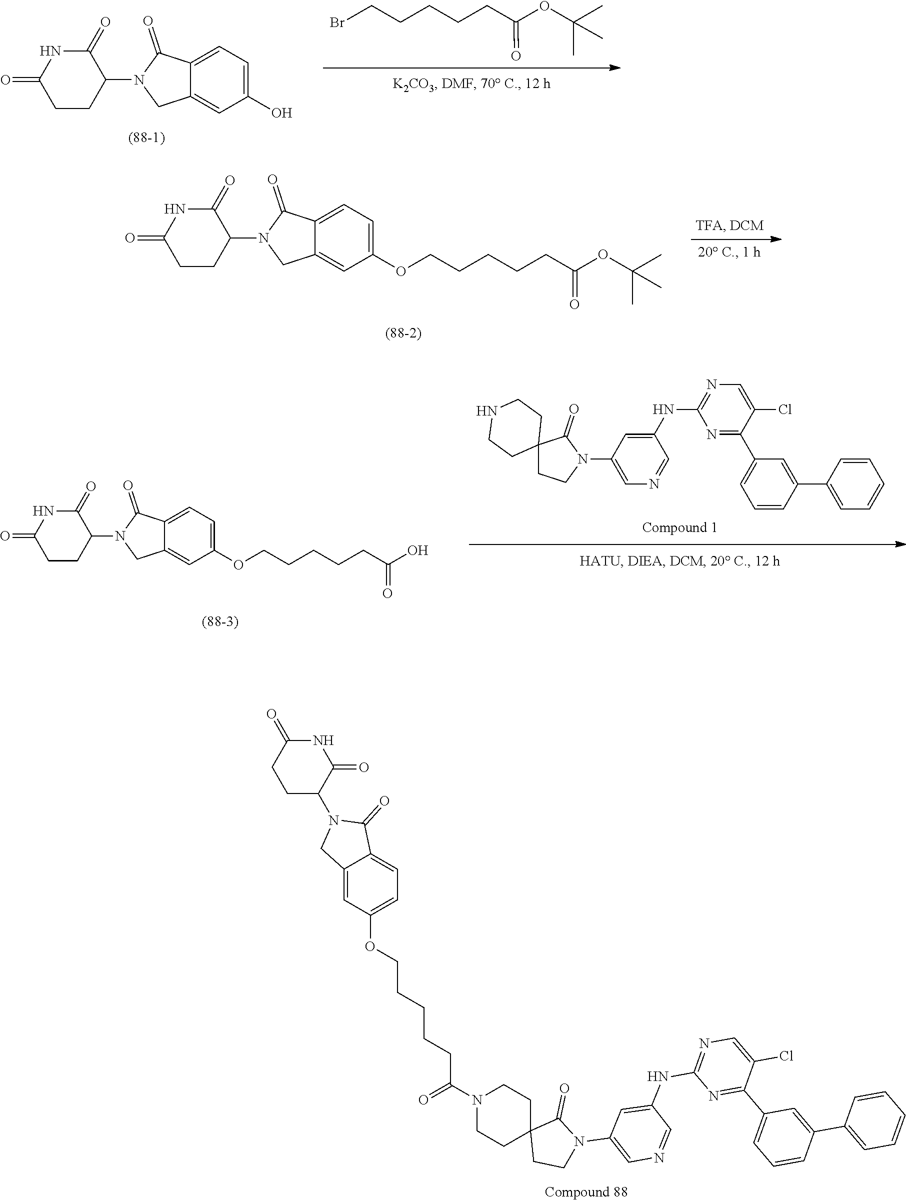 [SMILES: O=C1CCC(N2Cc3cc(O)ccc3C2=O)C(=O)N1 ; CC(C)(C)OC(=O)CCCCCBr ; CC(C)(C)OC(=O)CCCCCOc1ccc2c(c1)CN(C1CCC(=O)NC1=O)C2=O ; O=C(O)CCCCCOc1ccc2c(c1)CN(C1CCC(=O)NC1=O)C2=O ; O=C1N(c2cncc(Nc3ncc(Cl)c(-c4cccc(-c5ccccc5)c4)n3)c2)CCC12CCNCC2 ; O=C1CCC(N2Cc3cc(OCCCCCC(=O)N4CCC5(CC4)CCN(c4cncc(Nc6ncc(Cl)c(-c7cccc(-c8ccccc8)c7)n6)c4)C5=O)ccc3C2=O)C(=O)N1]

Synthesis of tert-butyl 6-((2-(2,6-dioxopiperidin-3-yl)-1-oxoisoindolin-5-yl)oxy)hexanoate (88-2). To a solution of 3-(5-hydroxy-1-oxoisoindolin-2-yl)piperidine-2,6-dione (88-1) (500 mg, 1.92 mmol, 1.00 eq) and tert-butyl 6-bromohexanoate (724 mg, 2.88 mmol, 1.50 eq) in DMF (5 mL) was added K₂CO₃ (797 mg, 5.76 mmol, 3.00 eq) at 20° C., the mixture was stirred at 70° C. for 12 h. LCMS showed 3-(5-hydroxy-1-oxoisoindolin-2-yl)piperidine-2,6-dione (88-1) remained and the desired mass was detected. H₂O (6 mL) was added to the mixture and the aqueous was extracted with DCM (3 mL*3), the combined organic layers were dried over Na₂SO₄ and then concentrated in vacuo. The residue was purified by prep-TLC (SiO₂, Petroleum ether:Ethyl acetate=0:1) to give the title compound (88-2) (250 mg, crude) as a white solid. LCMS (ES⁺): m/z 431.2 [M+H]⁺.

Synthesis of 6-((2-(2,6-dioxopiperidin-3-yl)-1-oxoisoindolin-5-yl)oxy)hexanoic acid (88-3). To a solution of tert-butyl 6-((2-(2,6-dioxopiperidin-3-yl)-1-oxoisoindolin-5-yl)oxy)hexanoate (88-2) (150 mg, 348 μmol, 1.00 eq) in DCM (5 mL) was added TFA (1.5 mL) at 20° C., the mixture was stirred at 20° C. for 1 h. LCMS showed the reaction was complete, tert-butyl 6-((2-(2,6-dioxopiperidin-3-yl)-1-oxoisoindolin-5-yl)oxy)hexanoate (88-2) was consumed, desired target MS was detected. The mixture was concentrated in vacuo to give the title compound (88-3) (260 mg, crude) as a colorless oil. LCMS (ES⁺): m/z 375.1 [M+H]⁺.

Synthesis of 3-(5-((6-(2-(5-((4-([1,1′-biphenyl]-3-yl)-5-chloropyrimidin-2-yl)amino)pyridin-3-yl)-1-oxo-2,8-diazaspiro[4.5]decan-8-yl)-6-oxohexyl)oxy)-1-oxoisoindolin-2-yl)piperidine-2,6-dione (Compound 88). To a solution of 6-((2-(2,6-dioxopiperidin-3-yl)-1-oxoisoindolin-5-yl)oxy)hexanoic acid (88-3) (130 mg, 346 μmol, 1.00 eq) and 2-(5-((4-([1,1′-biphenyl]-3-yl)-5-chloropyrimidin-2-yl)amino)pyridin-3-yl)-2,8-diazaspiro[4.5]decan-1-one (Compound 1) (393 mg, 346 μmol, 55.0% purity, 1.00 eq, TFA) in DCM (5 mL) were added HATU (263 mg, 693 μmol, 2.00 eq) and DIEA (268 mg, 2.08 mmol, 362 μL, 6.00 eq) at 20° C., the mixture was stirred at 20° C. for 12 h. LCMS showed the reaction was complete, 6-((2-(2,6-dioxopiperidin-3-yl)-1-oxoisoindolin-5-yl)oxy)hexanoic acid (88-3) was consumed, desired target MS was detected. The residue was purified by prep-HPLC (column: Phenomenex Luna C18 75*30 mm*3 um; mobile phase: [water (FA)-ACN]; B %: 30%-70%, 8 min) to give the crude product. The crude product was purified again by prep-HPLC (column: Waters Xbridge Prep OBD C18 150*40 mm*10 um; mobile phase: [water (NH₄HCO₃)-ACN]; B %: 55%-75%, 8 min) to give the title compound (Compound 88) (18.5 mg, 20.8 μmol, 7.84% yield, 97.6% purity) as a white solid. LCMS (ES⁺): m/z 867.3 [M+H]⁺.

### Example 89. Synthesis of N-(5-((4-([1,1′-biphenyl]-3-yl)-5-chloropyrimidin-2-yl)amino)pyridin-3-yl)-9-(3-((2-(2,6-dioxopiperidin-3-yl)-1,3-dioxoisoindolin-4-yl)oxy)propanamido)nonanamide (Compound 89)

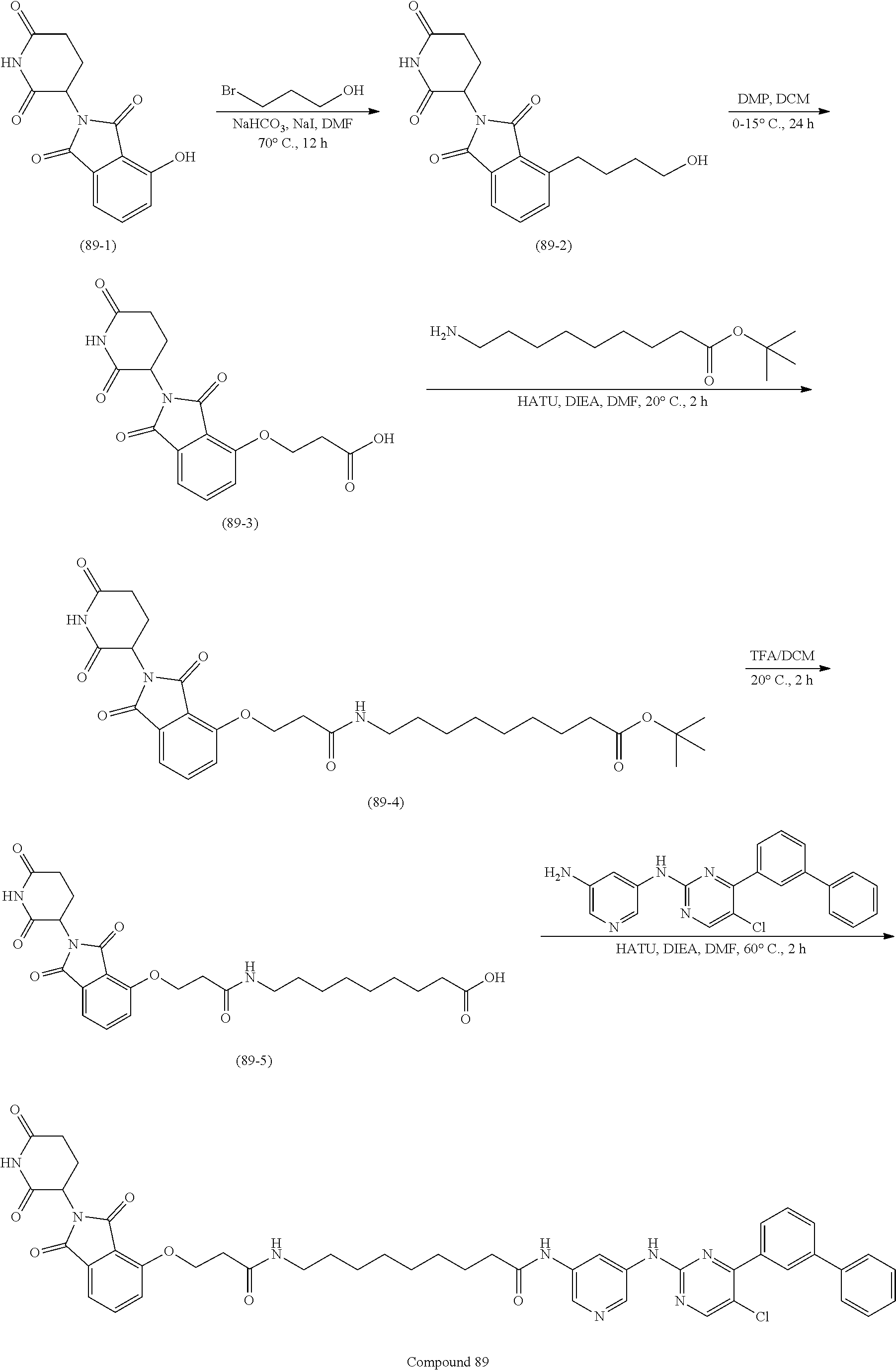

Synthesis of 2-(2,6-dioxopiperidin-3-yl)-4-(3-hydroxypropoxy)isoindoline-1,3-dione (89-2). To a stirred solution of 2-(2,6-dioxopiperidin-3-yl)-4-hydroxyisoindoline-1,3-dione (89-1) (3.00 g, 10.9 mmol, 1.00 eq) and 3-bromopropan-1-ol (4.56 g, 32.8 mmol, 2.96 mL, 3.00 eq) in DMF (30 mL) were added NaI (1.64 g, 10.9 mmol, 1.00 eq) and NaHCO₃ (1.84 g, 21.9 mmol, 851 μL, 2.00 eq) at 15° C. The resulting mixture was stirred at 70° C. for 12 h. TLC (SiO₂, PE:EtOAc=0:1) showed 2-(2,6-dioxopiperidin-3-yl)-4-hydroxyisoindoline-1,3-dione (89-1) was consumed and a new spot was formed. The mixture was diluted with H₂O (50 mL) and then extracted with EtOAc (30 mL*3). The combined organic layers were washed with saturated aqueous NaCl solution (50 mL), dried over Na₂SO₄, filtered and the filtrate was concentrated in vacuo. The crude product was triturated with DCM (10 mL) at 15° C. for 0.5 h and then filtered. The filter cake was dried in vacuo to give the title compound (89-2) (1.20 g, 3.61 mmol, 33.0% yield) as a white solid. LCMS (ES⁺): m/z 333.1 [M+H]⁺.

Synthesis of 3-((2-(2,6-dioxopiperidin-3-yl)-1,3-dioxoisoindolin-4-yl)oxy)propanoic acid (89-3). To a stirred solution of 2-(2,6-dioxopiperidin-3-yl)-4-(3-hydroxypropoxy)isoindoline-1,3-dione (89-2) (500 mg, 1.50 mmol, 1.00 eq) in DCM (10 mL) was added DMP (1.28 g, 3.01 mmol, 932 μL, 2.00 eq) at 0° C. The resulting mixture was stirred at 15° C. for 12 h. LCMS showed 2-(2,6-dioxopiperidin-3-yl)-4-(3-hydroxypropoxy)isoindoline-1,3-dione (89-2) remained and the desired mass was detected. To the mixture was added DMP (1.28 g, 3.01 mmol, 932 μL, 2.00 eq) at 0° C. The resulting mixture was stirred at 15° C. for 12 h. LCMS showed 2-(2,6-dioxopiperidin-3-yl)-4-(3-hydroxypropoxy)isoindoline-1,3-dione (89-2) was consumed completely and the desired mass was detected. The mixture was diluted with H₂O (30 mL) and then extracted with DCM (20 mL*3). The combined organic layers were dried over Na₂SO₄, filtered and the filtrate was concentrated in vacuo. The mixture was purified by prep-HPLC (column: Phenomenex Luna C18 75*30 mm*3 um; mobile phase: [water (TFA)-ACN]; B %: 1%-30%, 8 min) to give the title compound (89-3) (200 mg, 578 μmol, 38.4% yield) as a white solid. LCMS (ES⁺): m/z 347.0 [M+H]⁺.

Synthesis of tert-butyl 9-(3-((2-(2,6-dioxopiperidin-3-yl)-1,3-dioxoisoindolin-4-yl)oxy)propanamido)nonanoate (89-4). To a solution of 3-((2-(2,6-dioxopiperidin-3-yl)-1,3-dioxoisoindolin-4-yl)oxy)propanoic acid (89-3) (80.0 mg, 231 μmol, 1.00 eq) in DMF (2 mL) were added HATU (96.6 mg, 254 μmol, 1.10 eq) and DIEA (89.6 mg, 693 μmol, 121 μL, 3.00 eq) at 20° C. To the mixture was added tert-butyl 9-aminononanoate (53.0 mg, 231 μmol, 1.00 eq) at 20° C. and stirred at 20° C. for 2 h. LCMS showed 3-((2-(2,6-dioxopiperidin-3-yl)-1,3-dioxoisoindolin-4-yl)oxy)propanoic acid (89-3) was consumed and desired mass was detected. H₂O (3 mL) was added to the solution, then the mixture was extracted with EtOAc (3 mL*3). The combined organic layers were dried over Na₂SO₄, then concentrated in vacuo to give the crude product. The mixture was purified by prep-TLC (SiO₂, PE:EtOAc=0:1) to give the title compound (89-4) (40.0 mg, 71.7 μmol, 31.0% yield) as a white solid. LCMS (ES⁺): m/z 558.3 [M+H]⁺.

Synthesis of 9-(3-((2-(2,6-dioxopiperidin-3-yl)-1,3-dioxoisoindolin-4-yl)oxy)propanamido)nonanoic acid (89-5). A solution of tert-butyl 9-(3-((2-(2,6-dioxopiperidin-3-yl)-1,3-dioxoisoindolin-4-yl)oxy)propanamido)nonanoate (89-4) (40.0 mg, 71.7 μmol, 1.00 eq) in TFA (0.2 mL) and DCM (1 mL) was stirred at 20° C. for 2 h. LCMS showed tert-butyl 9-(3-((2-(2,6-dioxopiperidin-3-yl)-1,3-dioxoisoindolin-4-yl)oxy)propanamido)nonanoate (89-4) was consumed completely and desired compound was detected. The mixture was concentrated in vacuo to give the title compound (89-5) (40.0 mg, crude) as a yellow solid. LCMS (ES⁺): m/z 502.2 [M+H]⁺.

Synthesis of N-(5-((4-([1,1′-biphenyl]-3-yl)-5-chloropyrimidin-2-yl)amino)pyridin-3-yl)-9-(3-((2-(2,6-dioxopiperidin-3-yl)-1,3-dioxoisoindolin-4-yl)oxy)propanamido)nonanamide (Compound 89). To a solution of 9-(3-((2-(2,6-dioxopiperidin-3-yl)-1,3-dioxoisoindolin-4-yl)oxy)propanamido)nonanoic acid (89-5) (40.0 mg, 79.8 μmol, 1.00 eq) in DMF (1 mL) were added HATU (45.5 mg, 120 μmol, 1.50 eq) and DIEA (61.9 mg, 479 μmol, 83.4 μL, 6.00 eq) at 20° C. To the mixture was added N³-(4-([1,1′-biphenyl]-3-yl)-5-chloropyrimidin-2-yl)pyridine-3,5-diamine (29.8 mg, 79.8 μmol, 1.00 eq) (prepared as described in Example 58) at 20° C. and stirred at 60° C. for 2 h. LCMS showed 9-(3-((2-(2,6-dioxopiperidin-3-yl)-1,3-dioxoisoindolin-4-yl)oxy)propanamido)nonanoic acid (89-5) was consumed, and desired compound was detected. Water (3 mL) was added to the solution, then the mixture was extracted with EtOAc (3 mL*3), the combined organic layers were dried over Na₂SO₄, then concentrated in vacuo to give the crude product. The residue was purified by prep-HPLC (column: Phenomenex Luna C18 75*30 mm*3 um; mobile phase: [water (FA)-ACN]; B %: 30%-60%, 8 min) to give the title compound (Compound 89) (6.40 mg, 6.80 μmol, 8.52% yield, 91.0% purity) as a white solid. LCMS (ES⁺): m/z 857.3 [M+H]⁺.

### Example 90. Synthesis of Additional Compounds of Table 1

Compounds 92-119 may be prepared via methods analogous to those disclosed in the general synthetic schemes and examples.

Compounds 92, 93, 94, 95, 98, 103, 104, 105, 108, 111, 112, 113, 116, 117, and 118 may be prepared via methods analogous to those disclosed in the general synthetic schemes and examples, e.g., Examples 12, 13, 39, or 40.

Compounds 96, 99, 100, 101, 102, 106, 107, 109, 110, 114, 115, and 119 may be prepared via methods analogous to those disclosed in the general synthetic schemes and examples, e.g., Examples 41, 42, 43, or 44.

### Example 91. Synthesis of 1-(5-((5-chloro-4-(3-chlorophenyl)pyrimidin-2-yl)amino)pyridin-3-yl)pyrrolidin-2-one (Compound 92)

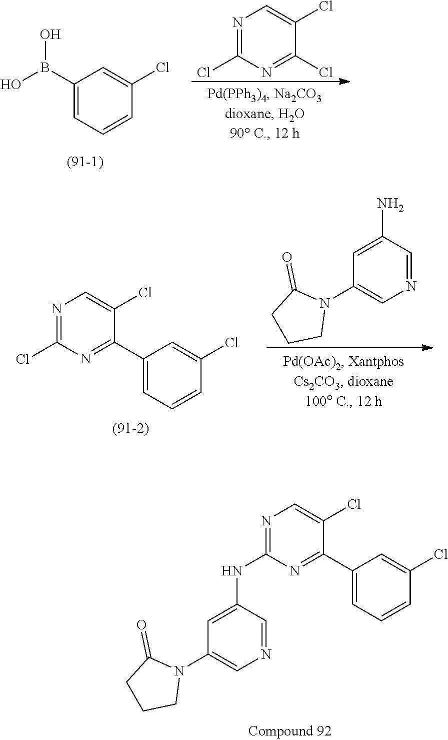 [SMILES: OB(O)c1cccc(Cl)c1 ; Clc1ncc(Cl)c(Cl)n1 ; Clc1cccc(-c2nc(Cl)ncc2Cl)c1 ; Nc1cncc(N2CCCC2=O)c1 ; O=C1CCCN1c1cncc(Nc2ncc(Cl)c(-c3cccc(Cl)c3)n2)c1]

Synthesis of 2,5-dichloro-4-(3-chlorophenyl)pyrimidine (91-2). To a solution of (3-chlorophenyl)boronic acid (91-1) (300 mg, 1.92 mmol, 1.00 eq) in dioxane (5 mL) and H₂O (0.5 mL) were added 2,4,5-trichloropyrimidine (704 mg, 3.84 mmol, 2.00 eq), Na₂CO₃ (407 mg, 3.84 mmol, 2.00 eq) and Pd(PPh₃)₄ (44.3 mg, 38.4 μmol, 0.0200 eq) at 20° C. under N₂. The mixture was stirred at 90° C. for 12 h under N₂ atmosphere. LCMS showed (3-chlorophenyl)boronic acid (91-1) was consumed and the desired mass was detected. Water (8 mL) was added to reaction, the aqueous phase was extracted with EtOAc (8 mL*3), the combined organic layers were dried over Na₂SO₄, filtered and the filtrate was concentrated under reduced pressure. The residue was purified by prep-TLC (SiO₂, Petroleum ether:Ethyl acetate=20:1) to give the title compound (91-2) (330 mg, 1.27 mmol, 66.3% yield) as a white solid. LCMS (ES⁺): m/z 258.9 [M+H]⁺.

Synthesis of 1-(5-((5-chloro-4-(3-chlorophenyl)pyrimidin-2-yl)amino)pyridin-3-yl)pyrrolidin-2-one (Compound 92). To a solution of 2,5-dichloro-4-(3-chlorophenyl)pyrimidine (91-2) (200 mg, 771 μmol, 0.800 eq) in dioxane (6 mL) were added 1-(5-amino-3-pyridyl)pyrrolidin-2-one (261 mg, 1.06 mmol, 72.0% purity, 1.10 eq), Pd(OAc)₂ (10.8 mg, 48.2 μmol, 0.0500 eq), Cs₂CO₃ (628 mg, 1.93 mmol, 2.00 eq) and Xantphos (16.7 mg, 28.9 μmol, 0.0300 eq) at 20° C. under N₂. The mixture was stirred at 100° C. for 12 h under N₂ atmosphere. LCMS showed 2,5-dichloro-4-(3-chlorophenyl)pyrimidine (91-2) was consumed and the desired mass was detected. Water (5 mL) was added to reaction, the aqueous phase was extracted with EtOAc (5 mL*3), the combined organic layers were dried over Na₂SO₄, filtered and the filtrate was concentrated under reduced pressure. The residue was purified by prep-HPLC (column: Phenomenex luna C18 100*40 mm*3 um; mobile phase: [water (FA)-ACN]; B %: 40%-80%, 8 min) to give the title compound (Compound 92) (65.6 mg, 163 μmol, 16.9% yield, 99.5% purity) as a white solid. LCMS (ES⁺): m/z 400.0 [M+H]⁺.

### Example 92. Synthesis of 1-(5-((5-chloro-4-(4-chlorophenyl)pyrimidin-2-yl)amino)pyridin-3-yl)pyrrolidin-2-one (Compound 93)

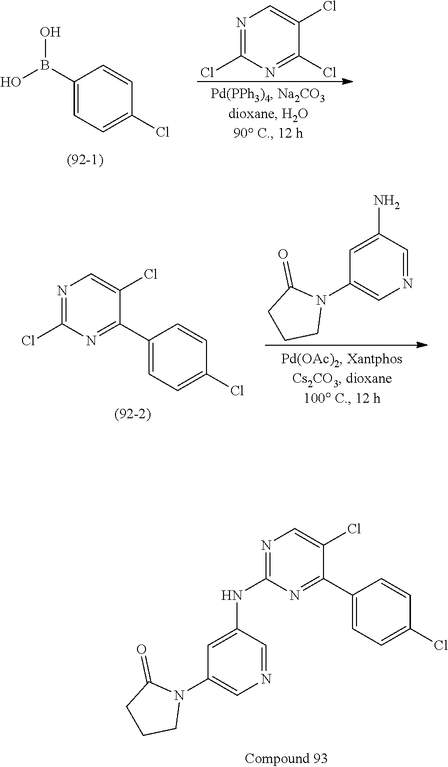 [SMILES: OB(O)c1ccc(Cl)cc1 ; Clc1ncc(Cl)c(Cl)n1 ; Clc1ccc(-c2nc(Cl)ncc2Cl)cc1 ; Nc1cncc(N2CCCC2=O)c1 ; O=C1CCCN1c1cncc(Nc2ncc(Cl)c(-c3ccc(Cl)cc3)n2)c1]

Synthesis of 2,5-dichloro-4-(4-chlorophenyl)pyrimidine (92-2). To a solution of (4-chlorophenyl)boronic acid (92-1) (300 mg, 1.92 mmol, 1.00 eq) in dioxane (6 mL) and H₂O (0.6 mL) were added 2,4,5-trichloropyrimidine (422 mg, 2.30 mmol, 1.20 eq), Na₂CO₃ (407 mg, 3.84 mmol, 2.00 eq) and Pd(PPh₃)₄ (44.3 mg, 38.4 μmol, 0.0200 eq) at 20° C. under N₂. The mixture was stirred at 90° C. for 12 h under N₂ atmosphere. LCMS showed (4-chlorophenyl)boronic acid (92-1) was consumed and the desired mass was detected. Water (8 mL) was added to reaction, the aqueous phase was extracted with EtOAc (8 mL*3), the combined organic layers were dried over Na₂SO₄, filtered and the filtrate was concentrated under reduced pressure. The residue was purified by prep-TLC (SiO₂, Petroleum ether:Ethyl acetate=20:1) to give the title compound (92-2) (342 mg, 1.32 mmol, 68.7% yield) as a white solid. LCMS (ES⁺): m/z 259.0 [M+H]⁺.

Synthesis of 1-(5-((5-chloro-4-(4-chlorophenyl)pyrimidin-2-yl)amino)pyridin-3-yl)pyrrolidin-2-one (Compound 93). To a solution of 2,5-dichloro-4-(4-chlorophenyl)pyrimidine (92-2) (200 mg, 771 μmol, 0.800 eq) in dioxane (6 mL) were added 1-(5-amino-3-pyridyl)pyrrolidin-2-one (261 mg, 1.06 mmol, 72.0% purity, 1.10 eq), Pd(OAc)₂ (10.8 mg, 48.2 μmol, 0.0500 eq), Cs₂CO₃ (628 mg, 1.93 mmol, 2.00 eq) and Xantphos (16.7 mg, 28.9 μmol, 0.0300 eq) at 20° C. under N₂. The mixture was stirred at 100° C. for 12 h under N₂ atmosphere. LCMS showed 2,5-dichloro-4-(4-chlorophenyl)pyrimidine (92-2) was consumed and the desired mass was detected. Water (5 mL) was added to reaction, the aqueous phase was extracted with EtOAc (5 mL*3), the combined organic layers were dried over Na₂SO₄, filtered and the filtrate was concentrated under reduced pressure. The residue was purified by prep-HPLC (column: Phenomenex luna C18 100*40 mm*3 um; mobile phase: [water (FA)-ACN]; B %: 40%-80%, 8 min) to give the title compound (Compound 93) (77.4 mg, 192 μmol, 19.9% yield, 99.1% purity) as a white solid. LCMS (ES⁺): m/z 400.0 [M+H]⁺.

### Example 93. Synthesis of 1-(5-((5-chloro-4-(p-tolyl)pyrimidin-2-yl)amino)pyridin-3-yl)pyrrolidin-2-one (Compound 94)

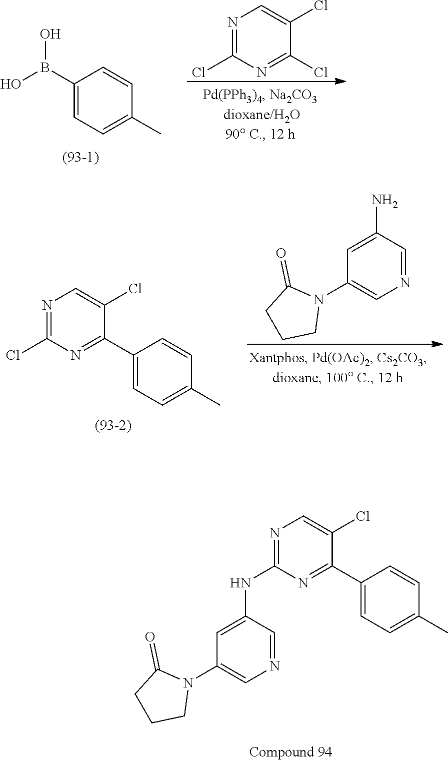

Synthesis of 2,5-dichloro-4-(p-tolyl)pyrimidine (93-2). To a solution of p-tolylboronic acid (93-1) (300 mg, 2.21 mmol, 1.00 eq) in dioxane (10 mL) and H₂O (1 mL) were added 2,4,5-trichloropyrimidine (809 mg, 4.41 mmol, 2.00 eq), Pd(PPh₃)₄ (51.0 mg, 44.1 μmol, 0.0200 eq) and Na₂CO₃ (468 mg, 4.41 mmol, 2.00 eq) at 25° C. under N₂ atmosphere. The mixture was stirred at 90° C. for 12 h. LCMS showed p-tolylboronic acid (93-1) was consumed and the desired mass was detected. The reaction mixture was concentrated under reduced pressure to remove solvent. The residue was purified by prep-TLC (SiO₂, Petroleum ether:Ethyl acetate=10:1) to give the title compound (93-2) (475 mg, 1.99 mmol, 90.0% yield) as a white solid. LCMS (ES⁺): m/z 239.0 [M+H]⁺.

Synthesis of 1-(5-((5-chloro-4-(p-tolyl)pyrimidin-2-yl)amino)pyridin-3-yl)pyrrolidin-2-one (Compound 94). To a solution of 2,5-dichloro-4-(p-tolyl)pyrimidine (93-2) (200 mg, 836 μmol, 0.800 eq) in dioxane (10 mL) were added 1-(5-amino-3-pyridyl)pyrrolidin-2-one (283 mg, 1.15 mmol, 72.0% purity, 1.10 eq), Xantphos (18.2 mg, 31.4 μmol, 0.0300 eq), Cs₂CO₃ (681 mg, 2.09 mmol, 2.00 eq) and Pd(OAc)₂ (11.7 mg, 52.3 μmol, 0.0500 eq) at 25° C. under N₂ atmosphere. The mixture was stirred at 100° C. for 12 h. LCMS showed 2,5-dichloro-4-(p-tolyl)pyrimidine (93-2) was consumed and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 94) (59.6 mg, 157 μmol, 15.0% yield, 100% purity) as a white solid. LCMS (ES⁺): m/z 380.2 [M+H]⁺.

### Example 94. Synthesis of 1-(5-((5-chloro-4-(2-chlorophenyl)pyrimidin-2-yl)amino)pyridin-3-yl)pyrrolidin-2-one (Compound 95)

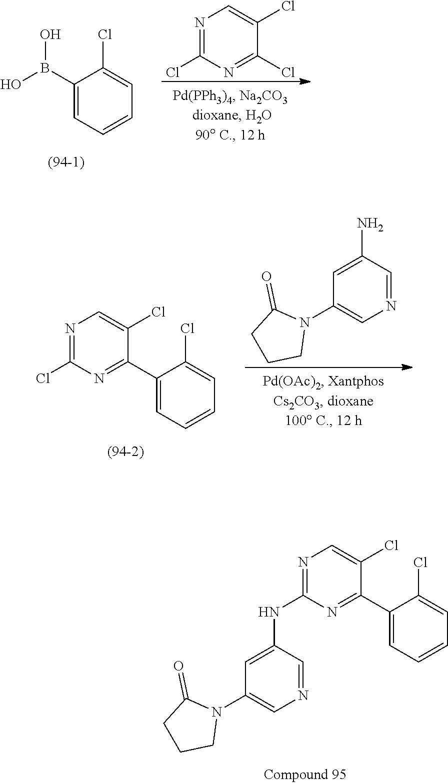

Synthesis of 2,5-dichloro-4-(2-chlorophenyl)pyrimidine (94-2). To a solution of (2-chlorophenyl)boronic acid (94-1) (300 mg, 1.92 mmol, 1.00 eq) in dioxane (6 mL) and H₂O (0.6 mL) were added 2,4,5-trichloropyrimidine (704 mg, 3.84 mmol, 2.00 eq), Na₂CO₃ (407 mg, 3.84 mmol, 2.00 eq) and Pd(PPh₃)₄ (44.3 mg, 38.4 μmol, 0.0200 eq) at 20° C. under N₂. The mixture was stirred at 90° C. for 12 h under N₂ atmosphere. LCMS showed (2-chlorophenyl)boronic acid (94-1) was consumed and the desired mass was detected. Water (15 mL) was added to reaction, the aqueous phase was extracted with EtOAc (15 mL*3), the combined organic layers were dried over Na₂SO₄, filtered and the filtrate was concentrated under reduced pressure. The residue was purified by prep-TLC (SiO₂, Petroleum ether:Ethyl acetate=20:1) to give the title compound (94-2) (240 mg, 925 μmol, 48.2% yield) as a white solid. LCMS (ES⁺): m/z 258.9 [M+H]⁺.

Synthesis of 1-(5-((5-chloro-4-(2-chlorophenyl)pyrimidin-2-yl)amino)pyridin-3-yl)pyrrolidin-2-one (Compound 95). To a solution of 2,5-dichloro-4-(2-chlorophenyl)pyrimidine (94-2) (240 mg, 925 μmol, 0.800 eq) in dioxane (8 mL) were added 1-(5-amino-3-pyridyl)pyrrolidin-2-one (313 mg, 1.27 mmol, 72.0% purity, 1.10 eq), Pd(OAc)₂ (13.0 mg, 57.8 μmol, 0.0500 eq), Cs₂CO₃ (753 mg, 2.31 mmol, 2.00 eq) and Xantphos (20.1 mg, 34.7 μmol, 0.0300 eq) at 20° C. under N₂. The mixture was stirred at 100° C. for 12 h under N₂ atmosphere. LCMS showed 2,5-dichloro-4-(2-chlorophenyl)pyrimidine (94-2) was consumed and the desired mass was detected. Water (5 mL) was added to reaction, the aqueous phase was extracted with EtOAc (5 mL*3), the combined organic layers were dried over Na₂SO₄, filtered and the filtrate was concentrated under reduced pressure. The residue was purified by prep-HPLC (column: Phenomenex luna C18 100*40 mm*3 um; mobile phase: [water (FA)-ACN]; B %: 30%-70%, 8 min) to give the title compound (Compound 95) (144 mg, 354 μmol, 30.6% yield, 98.3% purity) as a white solid. LCMS (ES⁺): m/z 400.0 [M+H]⁺.

### Example 95. Synthesis of 1-(5-((5-chloro-4-(piperidin-1-yl)pyrimidin-2-yl)amino)pyridin-3-yl)pyrrolidin-2-one (Compound 96)

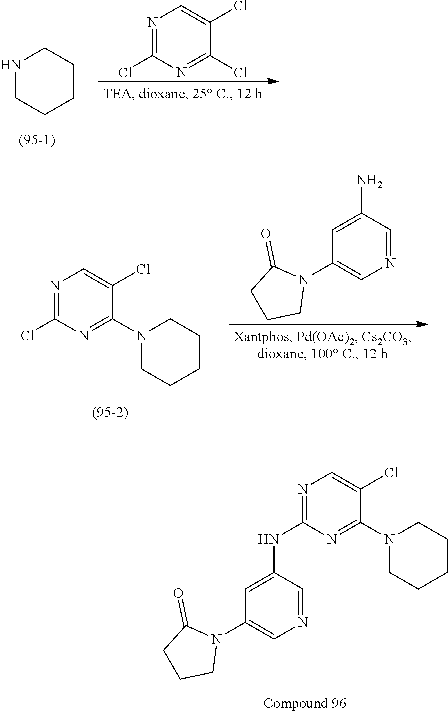 [SMILES: C1CCNCC1 ; Clc1ncc(Cl)c(Cl)n1 ; Clc1ncc(Cl)c(N2CCCCC2)n1 ; Nc1cncc(N2CCCC2=O)c1 ; O=C1CCCN1c1cncc(Nc2ncc(Cl)c(N3CCCCC3)n2)c1]

Synthesis of 2,5-dichloro-4-(piperidin-1-yl)pyrimidine (95-2). To a solution of piperidine (95-1) (306 mg, 3.60 mmol, 355 μL, 1.20 eq) in dioxane (6 mL) were added TEA (334 mg, 3.30 mmol, 459 μL, 1.10 eq) and 2,4,5-trichloropyrimidine (550 mg, 3.00 mmol, 1.00 eq) at 25° C. under N₂ atmosphere. The mixture was stirred at 25° C. for 12 h. LCMS showed piperidine 95-1 was consumed and the desired mass was detected. The reaction mixture was concentrated under reduced pressure to remove solvent. The residue was purified by prep-TLC (SiO₂, Petroleum ether:Ethyl acetate=10:1) to give the title compound (95-2) (300 mg, 1.29 mmol, 43.1% yield) as a white solid. LCMS (ES⁺): m/z 232.0 [M+H]⁺.

Synthesis of 1-(5-((5-chloro-4-(piperidin-1-yl)pyrimidin-2-yl)amino)pyridin-3-yl)pyrrolidin-2-one (Compound 96). To a solution of 2,5-dichloro-4-(piperidin-1-yl)pyrimidine (95-2) (200 mg, 862 μmol, 0.800 eq) in dioxane (5 mL) were added 1-(5-amino-3-pyridyl)pyrrolidin-2-one (292 mg, 1.18 mmol, 72.0% purity, 1.10 eq), Pd(OAc)₂ (12.1 mg, 53.9 μmol, 0.0500 eq), Cs₂CO₃ (702 mg, 2.15 mmol, 2.00 eq) and Xantphos (18.7 mg, 32.3 μmol, 0.0300 eq) at 25° C. under N₂ atmosphere. The mixture was stirred at 100° C. for 12 h. LCMS showed 2,5-dichloro-4-(piperidin-1-yl)pyrimidine (95-2) was consumed and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 96) (21.2 mg, 56.9 μmol, 5.28% yield, 100% purity) as a yellow solid. LCMS (ES⁺): m/z 373.1 [M+H]⁺.

### Example 96. Synthesis of 1-(5-((5-chloro-4-cyclohexylpyrimidin-2-yl)amino)pyridin-3-yl)pyrrolidin-2-one (Compound 97)

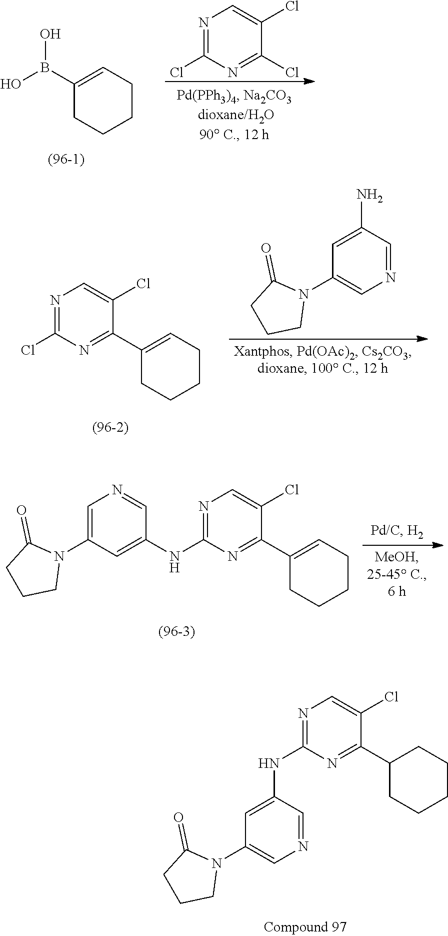

Synthesis of 2,5-dichloro-4-(cyclohex-1-en-1-yl)pyrimidine (96-2). To a solution of 2,4,5-trichloropyrimidine (874 mg, 4.76 mmol, 2.00 eq) in dioxane (10 mL) and H₂O (1 mL) were added cyclohex-1-en-1-ylboronic acid (96-1) (300 mg, 2.38 mmol, 1.00 eq), Pd(PPh₃)₄ (55.0 mg, 47.6 μmol, 0.0200 eq) and Na₂CO₃ (505 mg, 4.76 mmol, 2.00 eq) at 25° C. under N₂ atmosphere. The mixture was stirred at 90° C. for 12 h. LCMS showed cyclohex-1-en-1-ylboronic acid (96-1) was consumed and the desired mass was detected. Water (10 mL) was added to the mixture at 20° C. Then the mixture was extracted with EtOAc (15 mL). The organic layers were dried over anhydrous sodium sulfate, filtered and the filtrate was concentrated under reduced pressure to give a residue. The residue was purified by column chromatography (SiO₂, Petroleum ether:Ethyl acetate=1:0 to 10:1) to give the title compound (96-2) (300 mg, 1.31 mmol, 55.0% yield) as a colorless oil. LCMS (ES⁺): m/z 229.1 [M+H]⁺.

Synthesis of 1-(5-((5-chloro-4-(cyclohex-1-en-1-yl)pyrimidin-2-yl)amino)pyridin-3-yl)pyrrolidin-2-one (96-3). To a solution of 2,5-dichloro-4-(cyclohex-1-en-1-yl)pyrimidine (96-2) (181 mg, 786 μmol, 77.0% purity, 0.800 eq) in dioxane (5 mL) were added 2,5-dichloro-4-(cyclohexen-1-yl)pyrimidine (180 mg, 786 μmol, 0.800 eq), Pd(OAc)₂ (11.0 mg, 49.1 μmol, 0.0500 eq), Cs₂CO₃ (640 mg, 1.96 mmol, 2.00 eq) and Xantphos (17.1 mg, 29.5 μmol, 0.0300 eq) at 25° C. under N₂ atmosphere. The mixture was stirred at 100° C. for 12 h. LCMS showed 2,5-dichloro-4-(cyclohex-1-en-1-yl)pyrimidine (96-2) was consumed and the desired mass was detected. The reaction mixture was concentrated under reduced pressure to remove solvent. The residue was purified by prep-TLC (SiO₂, Ethyl acetate:Methanol=10:1) to give the title compound (96-3) (50.0 mg, 136 μmol, 13.8% yield) as a yellow solid. LCMS (ES⁺): m/z 370.2 [M+H]⁺.

Synthesis of 1-(5-((5-chloro-4-cyclohexylpyrimidin-2-yl)amino)pyridin-3-yl)pyrrolidin-2-one (Compound 97). 10% Pd/C (5.00 mg, 530 μmol, 50.0% purity) was added to MeOH (2 mL) at 25° C. under N₂ atmosphere, then 1-(5-((5-chloro-4-(cyclohex-1-en-1-yl)pyrimidin-2-yl)amino)pyridin-3-yl)pyrrolidin-2-one (96-3) (50.0 mg, 136 μmol, 1.00 eq) was added to the mixture at 25° C., the suspension was degassed and purged with H₂ for three times. The mixture was stirred at 25° C. for 1 h under H₂ (15 psi) atmosphere. LCMS showed 1-(5-((5-chloro-4-(cyclohex-1-en-1-yl)pyrimidin-2-yl)amino)pyridin-3-yl)pyrrolidin-2-one (96-3) remained, but no desired mass was detected. Then the mixture was stirred at 45° C. for 5 h. LCMS showed ˜18% of 1-(5-((5-chloro-4-(cyclohex-1-en-1-yl)pyrimidin-2-yl)amino)pyridin-3-yl)pyrrolidin-2-one (96-3) remained, and ˜13% of the desired mass was detected. The reaction mixture was filtered and the filtrate was concentrated under reduced pressure to give a residue. The residue was purified by prep-HPLC (column: Phenomenex Luna 80*30 mm*3 um; mobile phase: [water (FA)-ACN]; B %: 20%-45%, 8 min) to give the title compound (Compound 97) (2.00 mg, 5.05 μmol, 3.74% yield, 94.0% purity) as a white solid. LCMS (ES⁺): m/z 372.1 [M+H]⁺.

### Example 97. Synthesis of 1-(5-((5-chloro-4-(3-isopropylphenyl)pyrimidin-2-yl)amino)pyridin-3-yl)pyrrolidin-2-one (Compound 98)

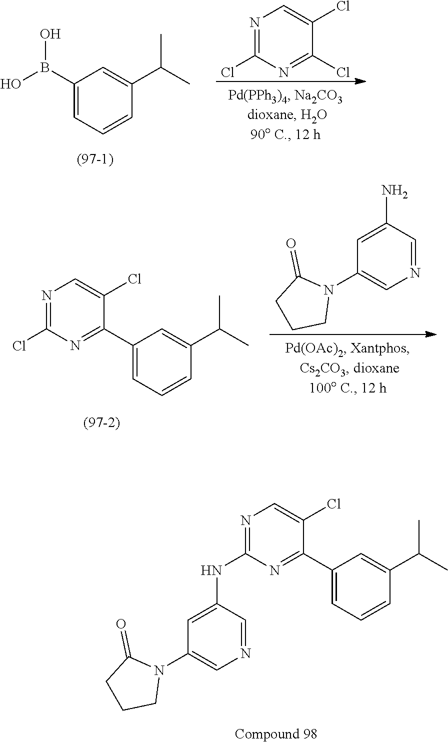

Synthesis of 2,5-dichloro-4-(3-isopropylphenyl) pyrimidine (97-2). To a solution of (3-isopropylphenyl)boronic acid (97-1) (300 mg, 1.83 mmol, 1.00 eq) and 2,4,5-trichloropyrimidine (671 mg, 3.66 mmol, 2.00 eq) in dioxane (5 mL) and H₂O (0.5 mL) were added Na₂CO₃ (388 mg, 3.66 mmol, 2.00 eq) and Pd(PPh₃)₄ (42.3 mg, 36.6 μmol, 0.0200 eq) at 15° C. The mixture was stirred at 90° C. for 12 h under N₂. LCMS showed (3-isopropylphenyl)boronic acid (97-1) was consumed and desired mass was detected. H₂O (3 mL) was added to the solution, then the mixture was extracted with EtOAc (3 mL*3), the combined organic layers were dried over Na₂SO₄, then concentrated in vacuo to give the crude product. The mixture was purified by prep-TLC (SiO₂, PE:EtOAc=15:1) to give the title compound (97-2) (100 mg, 374 μmol, 20.5% yield) as a yellow solid. LCMS (ES⁺): m/z 267.0 [M+H]⁺.

Synthesis of 1-(5-((5-chloro-4-(3-isopropylphenyl)pyrimidin-2-yl)amino)pyridin-3-yl)pyrrolidin-2-one (Compound 98). To a solution of 2,5-dichloro-4-(3-isopropylphenyl) pyrimidine (97-2) (100 mg, 374 μmol, 1.00 eq) and 1-(5-aminopyridin-3-yl)pyrrolidin-2-one (92.1 mg, 374 μmol, 72.0% purity, 1.00 eq) in dioxane (5 mL) were added Cs₂CO₃ (244 mg, 749 μmol, 2.00 eq), Pd(OAc)₂ (8.40 mg, 37.4 μmol, 0.100 eq) and Xantphos (21.7 mg, 37.4 μmol, 0.100 eq) at 15° C. The mixture was stirred at 100° C. for 12 h under N₂. LCMS showed 2,5-dichloro-4-(3-isopropylphenyl) pyrimidine (97-2) was consumed and desired mass was detected. H₂O (3 mL) was added to the solution, then the mixture was extracted with EtOAc (3 mL*3), the combined organic layers were dried over Na₂SO₄, then concentrated in vacuo to give the crude product. The residue was purified by prep-HPLC (column: Phenomenex Luna 80*30 mm*3 um; mobile phase: [water (TFA) ACN]; B %: 25%-60%, 8 min) to give the title compound (Compound 98) (16.0 mg, 36.1 μmol, 9.64% yield, 92.0% purity) as a white solid. LCMS (ES⁺): m/z 408.1 [M+H]⁺.

### Example 98. Synthesis of 1-(5-((5-chloro-4-(3-methoxypiperidin-1-yl)pyrimidin-2-yl)amino)pyridin-3-yl)pyrrolidin-2-one (Compound 99)

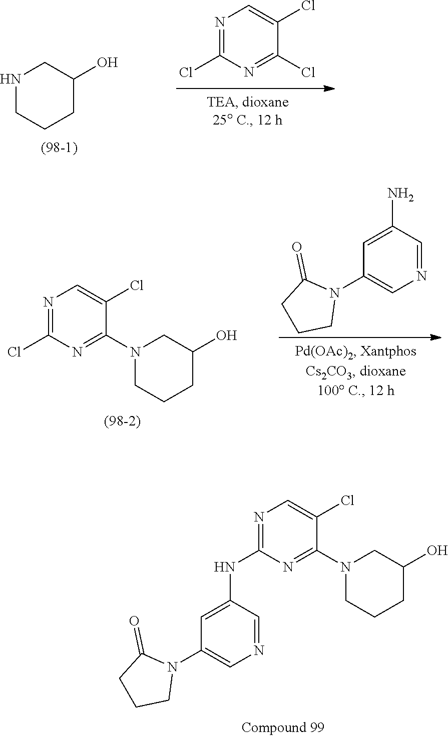 [SMILES: OC1CCCNC1 ; Clc1ncc(Cl)c(Cl)n1 ; OC1CCCN(c2nc(Cl)ncc2Cl)C1 ; Nc1cncc(N2CCCC2=O)c1 ; O=C1CCCN1c1cncc(Nc2ncc(Cl)c(N3CCCC(O)C3)n2)c1]

Synthesis of 1-(2,5-dichloropyrimidin-4-yl)piperidin-3-ol (98-2). To a stirred solution of piperidin-3-ol (98-1) (300 mg, 2.97 mmol, 1.20 eq) in dioxane (6 mL) were added TEA (275 mg, 2.72 mmol, 378 μL, 1.10 eq) and 2,4,5-trichloropyrimidine (453 mg, 2.47 mmol, 1.00 eq) at 25° C. The mixture was stirred at 25° C. for 12 h. LCMS showed that the desired mass was detected. The reaction mixture was concentrated under reduced pressure. The residue was purified by prep-TLC (SiO₂, Petroleum ether:Ethyl acetate=1:1) to give the title compound (98-1) (410 mg, crude) as a colorless oil. LCMS (ES⁺): m/z 248.0 [M+H]⁺.

Synthesis of 1-(5-((5-chloro-4-(3-methoxypiperidin-1-yl)pyrimidin-2-yl)amino)pyridin-3-yl)pyrrolidin-2-one (Compound 99). To a solution of 1-(2,5-dichloropyrimidin-4-yl)piperidin-3-ol (98-2) (410 mg, 1.65 mmol, 0.800 eq) in dioxane (10 mL) were added 1-(5-amino-3-pyridyl)pyrrolidin-2-one (523 mg, 2.27 mmol, 77.0% purity, 1.10 eq), Cs₂CO₃ (1.35 g, 4.13 mmol, 2.00 eq), Pd(OAc)₂ (23.2 mg, 103 μmol, 0.0500 eq) and Xantphos (35.9 mg, 62.0 μmol, 0.0300 eq) at 25° C. under N₂. The mixture was stirred at 100° C. for 12 h under N₂ atmosphere. LCMS showed 1-(2,5-dichloropyrimidin-4-yl)piperidin-3-ol (98-2) was consumed and the desired mass was detected. The reaction mixture was concentrated under reduced pressure. The residue was purified by prep-HPLC (column: Phenomenex luna C18 80*40 mm*3 um; mobile phase: [water (FA)-ACN]; B %: 15%-55%, 8 min) to give the title compound (Compound 99) (200 mg, 469 μmol, 22.7% yield, 91.1% purity) as a white solid. LCMS (ES⁺): m/z 389.0 [M+H]⁺.

### Example 99. Synthesis of 1-(5-((5-chloro-4-(3-methylpiperidin-1-yl)pyrimidin-2-yl)amino)pyridin-3-yl)pyrrolidin-2-one (Compound 100)

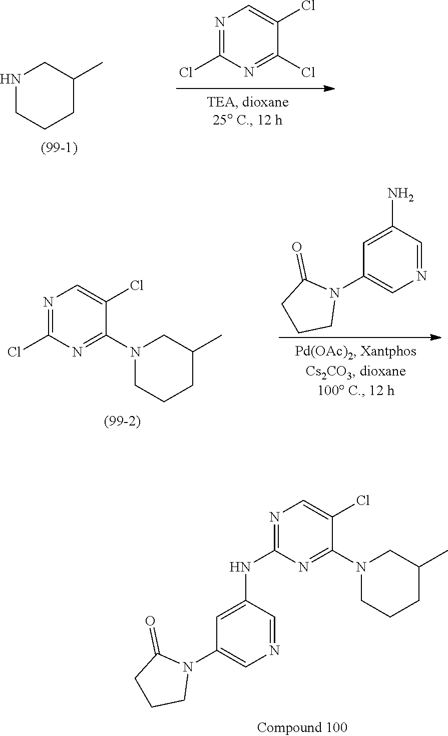

Synthesis of 2,5-dichloro-4-(3-methylpiperidin-1-yl)pyrimidine (99-2). To a solution of 3-methylpiperidine (99-1) (300 mg, 3.02 mmol, 355 μL, 1.20 eq) in dioxane (6 mL) were added TEA (280 mg, 2.77 mmol, 386 μL, 1.10 eq) and 2,4,5-trichloropyrimidine (462 mg, 2.52 mmol, 1.00 eq) at 25° C. The mixture was stirred at 25° C. for 12 h. LCMS showed 3-methylpiperidine (99-1) was consumed and the desired mass was detected. The reaction mixture was concentrated under reduced pressure to remove solvent. The residue was purified by prep-TLC (SiO₂, Petroleum ether:Ethyl acetate=10:1) to give the title compound (99-2) (435 mg, 1.77 mmol, 70.1% yield) as a white oil. LCMS (ES⁺): m/z 246.1 [M+H]⁺.

Synthesis of 1-(5-((5-chloro-4-(3-methylpiperidin-1-yl)pyrimidin-2-yl)amino)pyridin-3-yl)pyrrolidin-2-one (Compound 100). To a solution of 2,5-dichloro-4-(3-methylpiperidin-1-yl)pyrimidine (99-2) (200 mg, 737 μmol, 90.7% purity, 0.800 eq) in dioxane (4 mL) were added Cs₂CO₃ (600 mg, 1.84 mmol, 2.00 eq), Pd(OAc)₂ (10.3 mg, 46.1 μmol, 0.0500 eq), Xantphos (16.0 mg, 7.64 μmol, 0.0300 eq) and 1-(5-amino-3-pyridyl)pyrrolidin-2-one (233 mg, 1.01 mmol, 77.0% purity, 1.10 eq) at 25° C. under N₂ atmosphere. The mixture was stirred at 100° C. for 12 h. LCMS showed 2,5-dichloro-4-(3-methylpiperidin-1-yl)pyrimidine (99-2) was consumed and the desired mass was detected. The reaction mixture was concentrated under reduced pressure to remove solvent. The residue were purified by prep-HPLC (column: Phenomenex Luna C18 200*40 mm*10 um; mobile phase: [water (FA)-ACN]; B %: 25%-60%, 8 min) to give the title compound (Compound 100) (37.0 mg, 90.4 μmol, 9.81% yield, 94.5% purity) as a yellow solid. LCMS (ES⁺): m/z 387.1 [M+H]⁺.

### Example 100. Synthesis of 1-(5-((5-chloro-4-(3-(trifluoromethyl)piperidin-1-yl)pyrimidin-2-yl)amino)pyridin-3-yl)pyrrolidin-2-one (Compound 101)

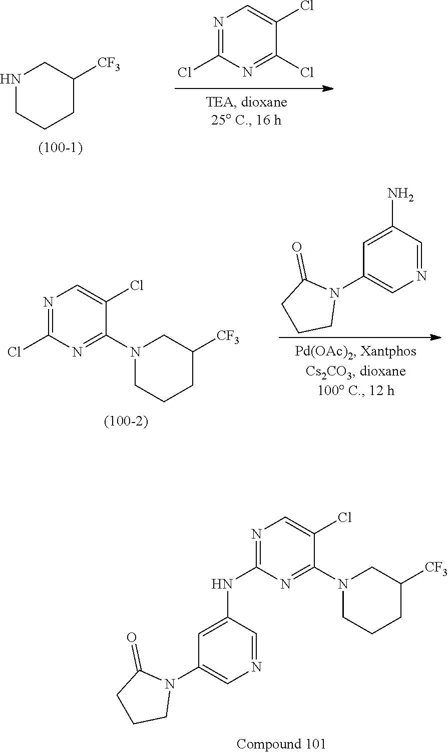 [SMILES: FC(F)(F)C1CCCNC1 ; Clc1ncc(Cl)c(Cl)n1 ; FC(F)(F)C1CCCN(c2nc(Cl)ncc2Cl)C1 ; Nc1cncc(N2CCCC2=O)c1 ; O=C1CCCN1c1cncc(Nc2ncc(Cl)c(N3CCCC(C(F)(F)F)C3)n2)c1]

Synthesis of 2,5-dichloro-4-(3-(trifluoromethyl)piperidin-1-yl)pyrimidine (100-2). To a solution of 3-(trifluoromethyl)piperidine (100-1) (300 mg, 1.33 mmol, 1.20 eq, HCl) in dioxane (6 mL) were added TEA (123 mg, 1.22 mmol, 169 μL, 1.10 eq) and 2,4,5-trichloropyrimidine (203 mg, 1.11 mmol, 1.00 eq) at 25° C. The mixture was stirred at 25° C. for 12 h. Then to the mixture was added TEA (123 mg, 1.22 mmol, 169 μL, 1.10 eq) at 25° C. The mixture was stirred at 25° C. for 4 h. LCMS showed ˜42.1% of 3-(trifluoromethyl)piperidine (100-1) was remaining and desired mass was detected. The reaction mixture was concentrated under reduced pressure to remove solvent. The residue was purified by prep-TLC (SiO₂, Petroleum ether:Ethyl acetate=20:1) to give the title compound (100-2) (435 mg, 1.77 mmol, 70.1% yield) as a colorless oil. LCMS (ES⁺): m/z 300.0 [M+H]⁺.

Synthesis of 1-(5-((5-chloro-4-(3-(trifluoromethyl)piperidin-1-yl)pyrimidin-2-yl)amino)pyridin-3-yl)pyrrolidin-2-one (Compound 101). To a solution of 2,5-dichloro-4-(3-(trifluoromethyl)piperidin-1-yl)pyrimidine (100-2) (139 mg, 605 μmol, 77.0% purity, 1.10 eq) in dioxane (4 mL) were added Cs₂CO₃ (358 mg, 1.10 mmol, 2.00 eq), Pd(OAc)₂ (6.17 mg, 27.5 μmol, 0.0500 eq), Xantphos (9.54 mg, 16.49 μmol, 0.0300 eq) and 2,5-dichloro-4-[3-(trifluoromethyl)-1-piperidyl]pyrimidine (132 mg, 440 μmol, 0.800 eq) at 25° C. under N₂ atmosphere. The mixture was stirred at 100° C. or 12 h. LCMS showed 2,5-dichloro-4-(3-(trifluoromethyl)piperidin-1-yl)pyrimidine (100-2) was consumed and the desired mass was detected. The reaction mixture was concentrated under reduced pressure to remove solvent. The residue were purified by prep-HPLC (column: Phenomenex C18 75*30 mm*3 um; mobile phase: [water (FA)-ACN]; B %: 20%-55%, 8 min) to give the title compound (Compound 101) (31.8 mg, 72.1 μmol, 13.1% yield, 100% purity) as a white solid. LCMS (ES⁺): m/z 441.1 [M+H]⁺.

### Example 101. Synthesis of 1-(5-((5-chloro-4-(4-methylpiperidin-1yl)pyrimidin-2-yl)amino)pyridin-3-yl)pyrrolidin-2-one (Compound 102)

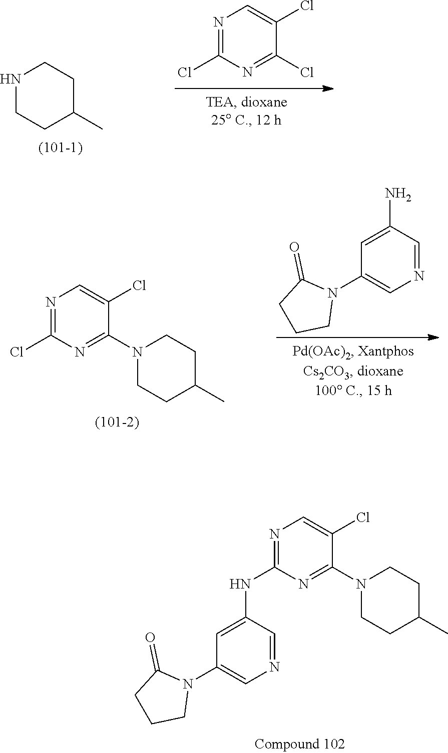

Synthesis of 2,5-dichloro-4-(4-methylpiperidin-1-yl)pyrimidine (101-2). To a solution of 4-methylpiperidine (101-1) (299 mg, 3.01 mmol, 356 μL, 1.20 eq) in dioxane (10 mL) were added TEA (279 mg, 2.76 mmol, 384 μL, 1.10 eq) and 2,4,5-trichloropyrimidine (460 mg, 2.51 mmol, 1.00 eq) at 25° C. The mixture was stirred at 25° C. for 12 h. LCMS showed 4-methylpiperidine (101-1) was consumed and the desired mass was detected. The reaction mixture was concentrated under reduced pressure to remove solvent. The residue was purified by prep-TLC (SiO₂, Petroleum ether:Ethyl acetate=10:1) to give the title compound (101-2) (596 mg, 2.42 mmol, 96.7% yield) as a white solid. LCMS (ES⁺): m/z 246.1 [M+H]⁺.

Synthesis of 1-(5-((5-chloro-4-(4-methylpiperidin-1yl)pyrimidin-2-yl)amino)pyridin-3-yl)pyrrolidin-2-one (Compound 102). To a solution of 2,5-dichloro-4-(4-methylpiperidin-1-yl)pyrimidine (101-2) (596 mg, 2.42 mmol, 0.800 eq) in dioxane (10 mL) were added Cs₂CO₃ (1.97 g, 6.05 mmol, 2.00 eq), 1-(5-amino-3-pyridyl)pyrrolidin-2-one (766 mg, 3.33 mmol, 77.0% purity, 1.10 eq), Xantphos (52.5 mg, 90.8 μmol, 0.0300 eq) and Pd(OAc)₂ (34.0 mg, 151 μmol, 0.0500 eq) at 25° C. under N₂ atmosphere. The mixture was stirred at 100° C. for 12 h. LCMS showed ˜53.0% of 2,5-dichloro-4-(4-methylpiperidin-1-yl)pyrimidine (101-2) was detected, ˜31.2% of 1-(5-amino-3-pyridyl)pyrrolidin-2-one were remaining and the desired mass was detected. Then Xantphos (52.5 mg, 90.8 μmol, 0.0300 eq), Cs₂CO₃ (1.97 g, 6.05 mmol, 2.00 eq) and Pd(OAc)₂ (34.0 mg, 151 μmol, 0.0500 eq) were added to the mixture at 25° C. under N₂ atmosphere. The mixture was stirred at 100° C. for 3 h. LCMS showed ˜17.9% of 2,5-dichloro-4-(4-methylpiperidin-1-yl)pyrimidine (101-2) was remaining and the desired mass was detected. The reaction mixture was concentrated under reduced pressure to remove solvent. The residue was purified by prep-HPLC (column: Waters Xbridge BEH C18 100*30 mm*10 um; mobile phase: [water (NH4HCO3)-ACN]; B %: 50%-80%, 8 min) to give the title compound (Compound 102) (162 mg, 413 μmol, 13.6% yield, 98.4% purity) as a yellow solid. LCMS (ES⁺): m/z 387.1 [M+H]⁺.

### Example 102. Synthesis of 1-(5-((5-chloro-4-(4-fluorophenyl)pyridin-2-yl)amino)pyridin-3-yl)pyrrolidin-2-one (Compound 103)

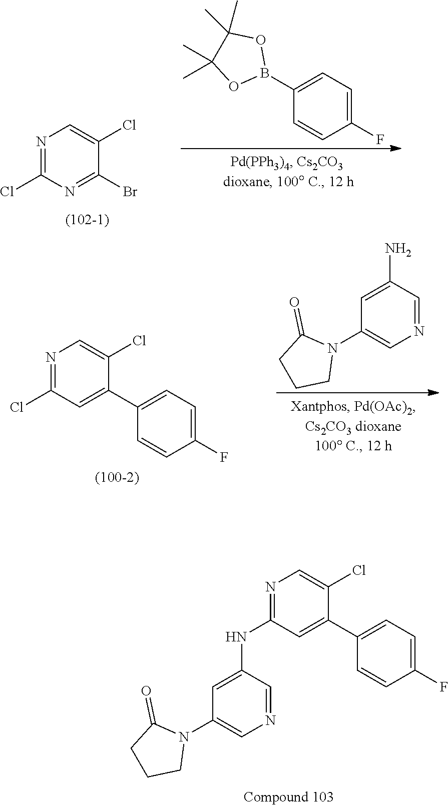 [SMILES: Clc1ncc(Cl)c(Br)n1 ; CC1(C)OB(c2ccc(F)cc2)OC1(C)C ; Fc1ccc(-c2cc(Cl)ncc2Cl)cc1 ; Nc1cncc(N2CCCC2=O)c1 ; O=C1CCCN1c1cncc(Nc2cc(-c3ccc(F)cc3)c(Cl)cn2)c1]

Synthesis of 2,5-dichloro-4-(4-fluorophenyl)pyridine (102-2). To a solution of 4-bromo-2,5-dichloropyridine (102-1) (1.00 g, 4.41 mmol, 1.00 eq), 2-(4-fluorophenyl)-4,4,5,5-tetramethyl-1,3,2-dioxaborolane (979 mg, 4.41 mmol, 1.00 eq) and Cs₂CO₃ (4.31 g, 13.2 mmol, 3.00 eq) in dioxane (10 mL) was added Pd(PPh₃)₄ (204 mg, 176 μmol, 0.0400 eq). The mixture was stirred at 100° C. for 12 h under N₂. LCMS showed 4-bromo-2, 5-dichloropyridine (102-1) was consumed and desired mass was detected. The reaction mixture was filtered and the filtrate was concentrated under reduced pressure to remove solvent. The residue was purified by flash silica gel chromatography (ISCO®; 10 g SepaFlash® Silica Flash Column, Eluent of 0-10% Ethyl acetate/Petroleum ether gradient @50 mL/min) to give the title compound (102-2) (600 mg, 2.48 mmol, 56.2% yield) as a yellow solid. LCMS (ES⁺): m/z 242.0 [M+H]⁺.

Synthesis of 1-(5-((5-chloro-4-(4-fluorophenyl)pyridin-2-yl)amino)pyridin-3-yl)pyrrolidin-2-one (Compound 103). To a solution of 2,5-dichloro-4-(4-fluorophenyl)pyridine (102-2) (200 mg, 826 μmol, 1.00 eq), 1-(5-amino-3-pyridyl)pyrrolidin-2-one (161 mg, 909 μmol, 1.10 eq), Cs₂CO₃ (673 mg, 2.07 mmol, 2.50 eq) and Xantphos (95.6 mg, 165 μmol, 0.200 eq) in dioxane (5 mL) was added Pd(OAc)₂ (18.6 mg, 82.6 μmol, 0.100 eq). The mixture was stirred at 100° C. for 12 h under N₂. LCMS showed 2, 5-dichloro-4-(4-fluorophenyl)pyridine (102-2) was consumed and desired mass was detected. The reaction mixture was concentrated. The residue was purified by prep-HPLC (column: Phenomenex Luna C18 75*30 mm*3 um; mobile phase: [water (FA)-ACN]; B %: 15%-55%, 8 min) to give the title compound (Compound 103) (26.3 mg, 65.5 μmol, 7.93% yield, 95.3% purity) as a white solid. LCMS (ES⁺): m/z 383.0 [M+H]⁺.

### Example 103. Synthesis of 1-(5-((5-chloro-4-(2, 4-difluorophenyl)pyridin-2-yl)amino)pyridin-3-yl)pyrrolidin-2-one (Compound 104)

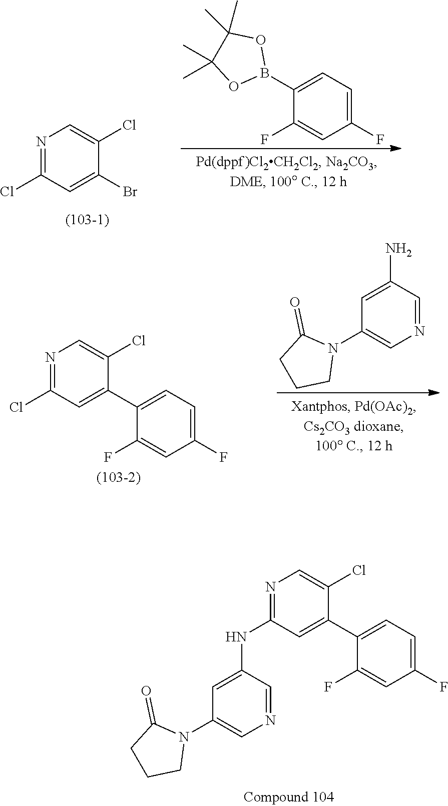 [SMILES: Clc1cc(Br)c(Cl)cn1 ; CC1(C)OB(c2ccc(F)cc2F)OC1(C)C ; Fc1ccc(-c2cc(Cl)ncc2Cl)c(F)c1 ; Nc1cncc(N2CCCC2=O)c1 ; O=C1CCCN1c1cncc(Nc2cc(-c3ccc(F)cc3F)c(Cl)cn2)c1]

Synthesis of 2, 5-dichloro-4-(2,4-difluorophenyl)pyridine (103-2). To a solution of 4-bromo-2,5-dichloropyridine (103-1) (1.00 g, 4.41 mmol, 1.00 eq) and 2-(2,4-difluorophenyl)-4,4,5,5-tetramethyl-1,3,2-dioxaborolane (1.06 g, 4.41 mmol, 1.00 eq) in DME (20 mL) were added Pd(dppf)Cl₂·CH₂Cl₂ (360 mg, 441 μmol, 0.100 eq) and Na₂CO₃ (1.40 g, 13.2 mmol, 3.00 eq). The mixture was stirred at 100° C. for 12 h under N₂. LCMS showed the desired mass was detected. The reaction mixture was filtered and the filtrate was concentrated under reduced pressure to give the title compound (103-2) (1.00 g, 3.85 mmol, 87.2% yield) as a yellow oil. LCMS (ES⁺): m/z 259.9 [M+H]⁺.

Synthesis of 1-(5-((5-chloro-4-(2,4-difluorophenyl)pyridin-2-yl)amino)pyridin-3-yl)pyrrolidin-2-one (Compound 104). To a solution of 2,5-dichloro-4-(2,4-difluorophenyl)pyridine (103-2) (300 mg, 1.15 mmol, 1.00 eq) and 1-(5-amino-3-pyridyl)pyrrolidin-2-one (307 mg, 1.73 mmol, 1.50 eq) in dioxane (5 mL) were added Cs₂CO₃ (1.13 g, 3.46 mmol, 3.00 eq), Xantphos (133 mg, 231 μmol, 0.200 eq) and Pd(OAc)₂ (25.9 mg, 115 μmol, 0.100 eq). The mixture was stirred at 100° C. for 12 h under N₂. LCMS showed 2,5-dichloro-4-(2,4-difluorophenyl)pyridine (103-2) remained and desired mass was detected. The reaction mixture was concentrated under reduced pressure to remove solvent. The residue was purified by prep-HPLC (column: Phenomenex Luna C18 75*30 mm*3 um; mobile phase: [water (FA)-ACN]; B %: 25%-65%, 8 min) to give the title compound (Compound 104) (25.0 mg, 58.2 μmol, 5.05% yield, 93.3% purity) as a yellow solid. LCMS (ES⁺): m/z 400.9 [M+H]⁺.

### Example 104. Synthesis of 1-(5-((5-chloro-4-(2-methylpyridin-4-yl)pyrimidin-2-yl)amino)pyridin-3-yl)pyrrolidin-2-one (Compound 105)

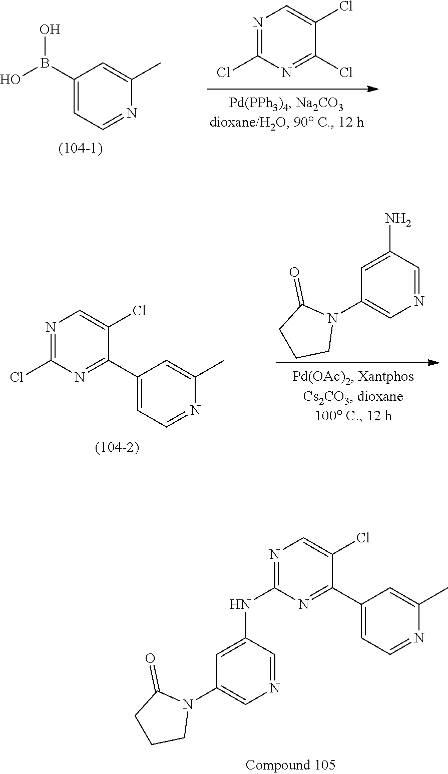

Synthesis of 2,5-dichloro-4-(2-methylpyridin-4-yl)pyrimidine (104-2). To a solution of (2-methylpyridin-4-yl)boronic acid (104-1) (300 mg, 2.19 mmol, 1.00 eq) in dioxane (6 mL) and H₂O (0.6 mL) were added Pd(PPh₃)₄ (50.6 mg, 43.8 μmol, 0.0200 eq), 2,4,5-trichloropyrimidine (803 mg, 4.38 mmol, 2.00 eq) and Na₂CO₃ (464 mg, 4.38 mmol, 2.00 eq) at 25° C. under N₂ atmosphere. The mixture was stirred at 90° C. for 12 h. LCMS showed (2-methylpyridin-4-yl)boronic acid (104-1) was consumed and the desired mass was detected. The reaction mixture was concentrated under reduced pressure to remove solvent. The residue was purified by prep-TLC (SiO₂, Petroleum ether:Ethyl acetate=2:1) to give the title compound (104-2) (350 mg, 1.46 mmol, 66.6% yield) as a brown solid. LCMS (ES⁺): m/z 240.0 [M+H]⁺.

Synthesis of 1-(5-((5-chloro-4-(2-methylpyridin-4-yl)pyrimidin-2-yl)amino)pyridin-3-yl)pyrrolidin-2-one (Compound 105). To a solution of 2,5-dichloro-4-(2-methylpyridin-4-yl)pyrimidine (104-2) (200 mg, 833 μmol, 0.800 eq) in dioxane (6 mL) were added Pd(OAc)₂ (11.7 mg, 52.1 μmol, 0.0500 eq), 1-(5-amino-3-pyridyl)pyrrolidin-2-one (264 mg, 1.15 mmol, 77.0% purity, 1.10 eq), Cs₂CO₃ (679 mg, 2.08 mmol, 2.00 eq) and Xantphos (18.1 mg, 31.2 μmol, 0.0300 eq) at 25° C. under N₂ atmosphere. The mixture was stirred at 100° C. for 12 h. LCMS showed 2,5-dichloro-4-(2-methylpyridin-4-yl)pyrimidine (104-2) was consumed and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH4HCO3)-ACN]; B %: 20%-55%, 8 min) to give the title compound (Compound 105) (23.6 mg, 58.9 μmol, 5.65% yield, 95.0% purity) as a brown solid. LCMS (ES⁺): m/z 381.0 [M+H]⁺.

### Example 105. Synthesis of 1-(5-((5-chloro-4-(2-methylpiperidin-1-yl)pyrimidin-2-yl)amino)pyridin-3-yl)pyrrolidin-2-one (Compound 106)

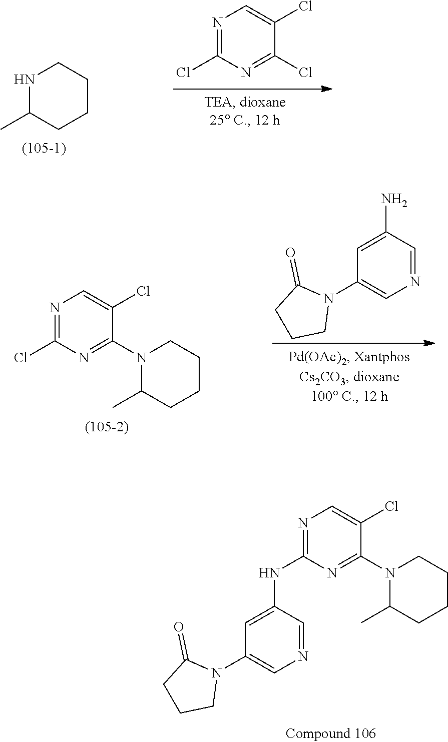

Synthesis of 2,5-dichloro-4-(2-methylpiperidin-1-yl)pyrimidine (105-2). To a solution of 2-methylpiperidine (105-1) (300 mg, 3.02 mmol, 358 μL, 1.20 eq) in dioxane (6 mL) were added TEA (281 mg, 2.77 mmol, 386 μL, 1.10 eq) and 2,4,5-trichloropyrimidine (462 mg, 2.52 mmol, 1.00 eq) at 25° C. The mixture was stirred at 25° C. for 12 h. LC-MS showed that the desired mass was detected. The reaction mixture was concentrated under reduced pressure to remove solvent. The residue was purified by prep-TLC (SiO₂, Petroleum ether:Ethyl acetate=20:1) to give the title compound (105-2) (450 mg, crude) as a white solid. LCMS (ES⁺): m/z 246.2 [M+H]⁺.

Synthesis of 1-(5-((5-chloro-4-(2-methylpiperidin-1-yl)pyrimidin-2-yl)amino)pyridin-3-yl)pyrrolidin-2-one (Compound 106). To a solution of 2,5-dichloro-4-(2-methylpiperidin-1-yl)pyrimidine (105-2) (250 mg, 1.02 mmol, 0.800 eq) in dioxane (10 mL) were added Cs₂CO₃ (827 mg, 2.54 mmol, 2.00 eq), Xantphos (22.0 mg, 38.1 μmol, 0.0300 eq), 1-(5-amino-3-pyridyl)pyrrolidin-2-one (321 mg, 1.40 mmol, 77.0% purity, 1.10 eq) and Pd(OAc)₂ (14.3 mg, 63.5 μmol, 0.0500 eq) at 25° C. under N₂. The mixture was stirred at 100° C. for 12 h under N₂ atmosphere. LCMS showed 2,5-dichloro-4-(2-methylpiperidin-1-yl)pyrimidine (105-2) was consumed mostly and the desired mass was detected. The reaction mixture was filtered and the filtrate was concentrated under reduced pressure. The residue was purified by prep-HPLC (column: Phenomenex Luna C18 75*30 mm*3 um; mobile phase: [water (FA)-ACN]; B %: 30%-60%, 8 min) to give the title compound (Compound 106) (123 mg, 313 μmol, 24.7% yield, 98.7% purity) as a white solid. LCMS (ES⁺): m/z 387.0 [M+H]⁺.

### Example 106. Synthesis of 1-(5-((5-chloro-4-morpholinopyrimidin-2-yl)amino)pyridin-3-yl)pyrrolidin-2-one (Compound 107)

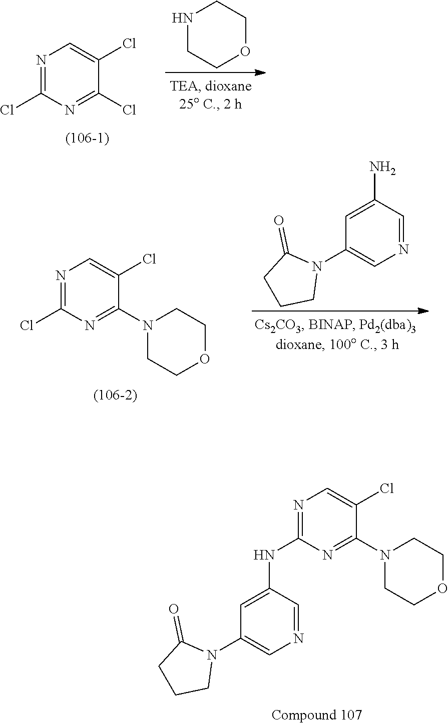 [SMILES: Clc1ncc(Cl)c(Cl)n1 ; C1COCCN1 ; Clc1ncc(Cl)c(N2CCOCC2)n1 ; Nc1cncc(N2CCCC2=O)c1 ; O=C1CCCN1c1cncc(Nc2ncc(Cl)c(N3CCOCC3)n2)c1]

Synthesis of 4-(2,5-dichloropyrimidin-4-yl)morpholine (106-2). To a stirred solution of 2,4,5-trichloropyrimidine (106-1) (500 mg, 2.73 mmol, 1.00 eq), morpholine (237 mg, 2.73 mmol, 240 μL, 1.00 eq) in dioxane (10 mL) was added TEA (828 mg, 8.18 mmol, 1.14 mL, 3.00 eq). The mixture was stirred at 25° C. for 2 h. LCMS showed the desired compound was detected. The reaction mixture was concentrated to give the title compound (106-2) (740 mg, 2.72 mmol, 100% yield, 86.2% purity) as a white solid, and it was used into the next step without further purification. LCMS (ES⁺): m/z 234.0 [M+H]⁺.

Synthesis of 1-(5-((5-chloro-4-morpholinopyrimidin-2-yl)amino)pyridin-3-yl)pyrrolidin-2-one (Compound 107). To a stirred solution of 4-(2,5-dichloropyrimidin-4-yl)morpholine (106-2) (300 mg, 1.28 mmol, 1.00 eq) and 1-(5-amino-3-pyridyl)pyrrolidin-2-one (250 mg, 1.41 mmol, 1.10 eq) in dioxane (10 mL) were added Cs₂CO₃ (836 mg, 2.57 mmol, 2.00 eq), BINAP (47.9 mg, 77.0 μmol, 0.0600 eq) and Pd₂(dba)₃ (47.0 mg, 51.3 μmol, 0.0400 eq). The mixture was stirred at 100° C. for 3 h under N₂. LCMS showed the desired compound was detected. The reaction mixture was concentrated. The residue was purified by prep-HPLC (column: Phenomenex luna C18 80*40 mm*3 um; mobile phase: [water (FA)-ACN]; B %: 1%-40%, 8 min) to give the title compound (Compound 107) (108 mg, 279 μmol, 21.8% yield, 97.2% purity) as a white solid. LCMS (ES⁺): m/z 375.1 [M+H]⁺.

### Example 107. Synthesis of 1-(5-((5-chloro-4-(2,4-difluorophenyl)pyrimidin-2-yl)amino)pyridin-3-yl)pyrrolidin-2-one (Compound 108)

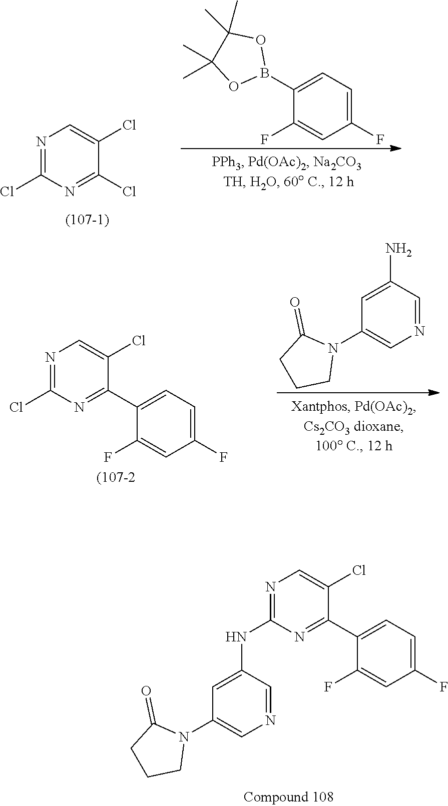 [SMILES: Clc1ncc(Cl)c(Cl)n1 ; CC1(C)OB(c2ccc(F)cc2F)OC1(C)C ; Fc1ccc(-c2nc(Cl)ncc2Cl)c(F)c1 ; Nc1cncc(N2CCCC2=O)c1 ; O=C1CCCN1c1cncc(Nc2ncc(Cl)c(-c3ccc(F)cc3F)n2)c1]

Synthesis of 2,5-dichloro-4-(2,4-difluorophenyl)pyrimidine (107-2). To a solution of 2,4,5-trichloropyrimidine (107-1) (1.00 g, 5.45 mmol, 1.00 eq) and 2-(2,4-difluorophenyl)-4,4,5,5-tetramethyl-1,3,2-dioxaborolane (1.31 g, 5.45 mmol, 1.00 eq) in THF (10 mL) and H₂O (2 mL) were added Na₂CO₃ (1.16 g, 10.9 mmol, 2.00 eq), PPh₃ (71.5 mg, 273 μmol, 0.0500 eq) and Pd(OAc)₂ (61.2 mg, 273 μmol, 0.0500 eq). The mixture was stirred at 60° C. for 12 h under N₂. LCMS showed desired MS was detected. The reaction mixture was diluted with H₂O (10 mL) and extracted with ethyl acetate (10 mL*3). The combined organic layers were dried over Na₂SO₄, filtered and concentrated under reduced pressure to give a residue. The residue was purified by column chromatography (SiO₂, Petroleum ether:Ethyl acetate=20:1 to 10:1) to give the title compound (107-2) (1.30 g, 4.98 mmol, 91.3% yield) as a white solid. LCMS (ES⁺): m/z 261.0 [M+H]⁺.

Synthesis of 1-(5-((5-chloro-4-(2,4-difluorophenyl)pyrimidin-2-yl)amino)pyridin-3-yl)pyrrolidin-2-one (Compound 108). To a solution of 2,5-dichloro-4-(2,4-difluorophenyl)pyrimidine (107-2) (100 mg, 383 μmol, 1.00 eq) and 1-(5-amino-3-pyridyl)pyrrolidin-2-one (74.7 mg, 421 μmol, 1.10 eq) in dioxane (10 mL) were added Cs₂CO₃ (250 mg, 766 μmol, 2.00 eq), Pd(OAc)₂ (4.30 mg, 19.2 μmol, 0.0500 eq) and Xantphos (22.2 mg, 38.3 μmol, 0.100 eq). The mixture was stirred at 100° C. for 12 h under N₂. LCMS showed desired MS was detected. The reaction mixture was concentrated under reduced pressure to give a residue. The residue was purified by prep-HPLC (column: 3_Phenomenex Luna C18 75*30 mm*3 um; mobile phase: [water (FA)-ACN]; B %: 15%-55%, 8 min) to give the title compound (Compound 108) (25.0 mg, 62.2 μmol, 16.2% yield, 100.00% purity) as a white solid. LCMS (ES⁺): m/z 401.8 [M+H]⁺.

### Example 108. Synthesis of N-butyl-1-(5-chloro-2-((5-(2-oxopyrrolidin-1-yl)pyridin-3-yl)amino)pyrimidin-4-yl)piperidine-3-carboxamide (Compound 109)

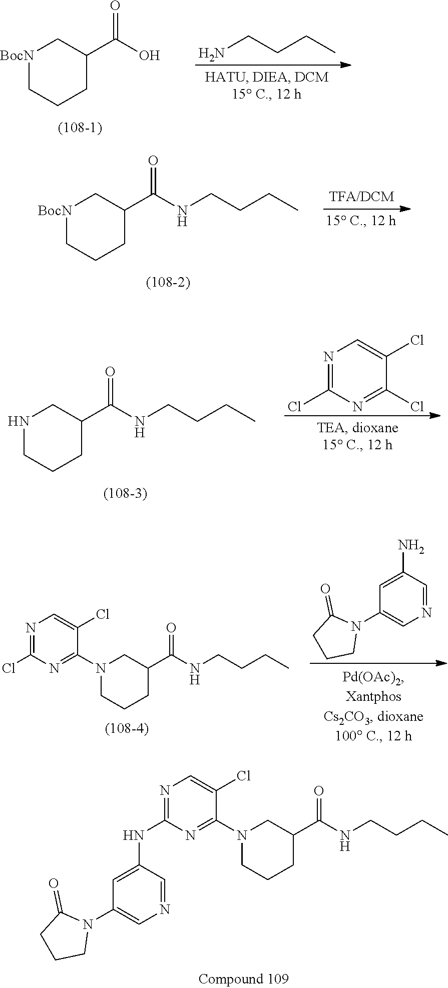

Synthesis of tert-butyl 3-(butylcarbamoyl)piperidine1-carboxylate (108-2). To a solution of 1-(tert-butoxycarbonyl)piperidine-3-carboxylic acid (108-1) (500 mg, 2.18 mmol, 1.00 eq) and DIEA (564 mg, 4.36 mmol, 760 μL, 2.00 eq) in DCM (6 mL) were added HATU (912 mg, 2.40 mmol, 1.10 eq) and butan-1-amine (191 mg, 2.62 mmol, 259 μL, 1.20 eq) at 15° C. The resulting mixture was stirred at 15° C. for 12 h. LCMS showed 1-(tert-butoxycarbonyl)piperidine-3-carboxylic acid (108-1) was consumed and the desired mass was detected. The mixture was concentrated in vacuo to give the crude product. The mixture was purified by prep-TLC (SiO₂, PE:EtOAc=1:1) to give the title compound (108-2) (500 mg, 1.76 mmol, 80.6% yield) as a yellow solid. LCMS (ES⁺): m/z 285.2 [M+H]⁺.

Synthesis of N-butylpiperidine-3carboxamide (108-3). The solution of tert-butyl 3-(butylcarbamoyl)piperidine1-carboxylate (108-2) (500 mg, 1.76 mmol, 1.00 eq) in DCM (6 mL) and TFA (2 mL) was stirred at 15° C. for 12 h. LCMS showed tert-butyl 3-(butylcarbamoyl)piperidine1-carboxylate (108-2) was consumed and the desired mass was detected. The mixture was concentrated in vacuo to give the title compound (108-3) (520 mg, crude, TFA) as a yellow oil. LCMS (ES⁺): m/z 185.2 [M+H]⁺.

Synthesis of N-butyl-1-(2,5-dichloropyrimidin-4yl)piperidine-3-carboxamide (108-4). To a solution of N-butylpiperidine-3carboxamide (108-3) (520 mg, 1.74 mmol, 1.00 eq, TFA) in dioxane (5 mL) were added TEA (264 mg, 2.61 mmol, 363 μL, 1.50 eq) and 2,4,5-trichloropyrimidine (319 mg, 1.74 mmol, 1.00 eq) at 15° C. The resulting mixture was stirred at 15° C. for 12 h. LCMS showed N-butylpiperidine-3carboxamide (108-3) was consumed and the desired mass was detected. The mixture was diluted with H₂O (10 mL) and then extracted with EtOAc (5 mL*3). The combined organic layers were dried over Na₂SO₄, filtered and the filtrate was concentrated in vacuo. The mixture was purified by prep-TLC (SiO₂, PE:EtOAc=10:1) to give the title compound 108-4 (300 mg, 906 μmol, 52.1% yield) as a yellow solid. LCMS (ES⁺): m/z 331.1 [M+H]⁺.

Synthesis of N-butyl-1-(5-chloro-2-((5-(2-oxopyrrolidin-1-yl)pyridin-3-yl)amino)pyrimidin-4-yl)piperidine-3-carboxamide (Compound 109). To a solution of N-butyl-1-(2,5-dichloropyrimidin-4yl)piperidine-3-carboxamide (108-4) (260 mg, 785 μmol, 1.00 eq) and 1-(5-aminopyridin-3-yl)pyrrolidin-2-one (199 mg, 785 μmol, 70.0% purity, 1.00 eq) in dioxane (5 mL) were added Xantphos (13.6 mg, 23.6 μmol, 0.0300 eq), Cs₂CO₃ (511 mg, 1.57 mmol, 2.00 eq) and Pd(OAc)₂ (8.81 mg, 39.3 μmol, 0.0500 eq) at 25° C. The resulting mixture was stirred at 100° C. for 12 h under N₂. LCMS showed N-butyl-1-(2,5-dichloropyrimidin-4yl)piperidine-3-carboxamide (108-4) was consumed and the desired mass was detected. The mixture was diluted with H₂O (10 mL) and then extracted with EtOAc (5 mL*3). The combined organic layers were dried over Na₂SO₄, filtered and the filtrate was concentrated in vacuo. The residue was purified by acidic prep-HPLC (column: Phenomenex Luna C18 200*40 mm*10 um; mobile phase: [water (FA)-ACN]; B %: 35%-70%, 8 min) to give the title compound (Compound 109) (47.3 mg, 99.1 μmol, 12.6% yield, 98.9% purity) as a white solid. LCMS (ES⁺): m/z 472.1 [M+H]⁺.

### Example 109. Synthesis of N-(1-(5-chloro-2-((5-(2-oxopyrrolidin-1-yl)pyridin-3-yl)amino)pyrimidin-4-yl)piperidin-3-yl)pentanamide (Compound 110)

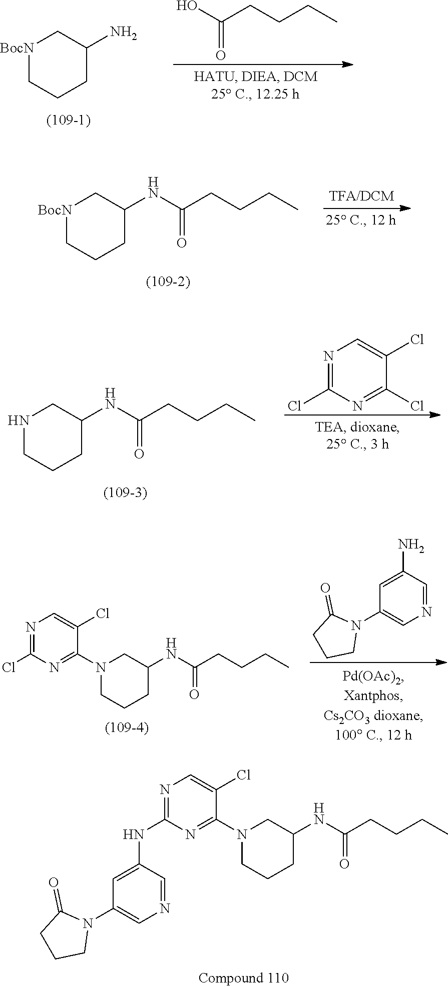

Synthesis of tert-butyl 3-pentanamidopiperidine-1-carboxylate (109-2). To a solution of pentanoic acid (1.02 g, 9.99 mmol, 1.09 mL, 1.00 eq) in DCM (30 mL) were added HATU (4.18 g, 11.0 mmol, 1.10 eq) and DIEA (5.16 g, 39.9 mmol, 6.96 mL, 4.00 eq) at 25° C. The mixture was stirred at 25° C. for 15 min. Then tert-butyl 3-aminopiperidine-1-carboxylate 109-1 (2.00 g, 9.99 mmol, 1.00 eq) was added to the mixture at 25° C. The mixture was stirred at 25° C. for 12 h. LCMS showed tert-butyl 3-aminopiperidine-1-carboxylate (109-1) was consumed and the desired mass was detected. The reaction mixture was concentrated under reduced pressure to remove solvent. The residue was purified by column chromatography (SiO₂, Petroleum ether:Ethyl acetate=20:1 to 1:1) to give the title compound (109-2) (2.84 g, 9.99 mmol, 100% yield) as an orange solid. LCMS (ES⁺): m/z 229.2 [M+H−56]⁺.

Synthesis of N-(piperidin-3-yl)pentanamide (109-3). To a solution of tert-butyl 3-pentanamidopiperidine-1-carboxylate (109-2) (400 mg, 1.41 mmol, 1.00 eq) in DCM (9 mL) was added TFA (4.62 g, 40.5 mmol, 3.00 mL, 28.8 eq) at 25° C. The mixture was stirred at 25° C. for 12 h. LCMS showed tert-butyl 3-pentanamidopiperidine-1-carboxylate (109-2) was consumed and the desired mass was detected. The reaction mixture was concentrated under reduced pressure to remove solvent to give the title compound (109-3) (419 mg, crude, TFA) as a brown oil. LCMS (ES⁺): m/z 185.2 [M+H]⁺.

Synthesis of N-(1-(2,5-dichloropyrimidin-4-yl)piperidin-3-yl)pentanamide (109-4). To a solution of N-(piperidin-3-yl)pentanamide (109-3) (418 mg, 1.40 mmol, 1.00 eq, TFA) in dioxane (15 mL) were added 2,4,5-trichloropyrimidine (257 mg, 1.40 mmol, 1.00 eq) and TEA (312 mg, 3.08 mmol, 430 μL, 2.20 eq) at 25° C. The mixture was stirred at 25° C. for 3 h. LCMS showed N-(piperidin-3-yl)pentanamide (109-3) was consumed and the desired mass was detected. The reaction mixture was concentrated under reduced pressure to remove solvent. The residue was purified by prep-TLC (SiO₂, Petroleum ether:Ethyl acetate=3:1) to give the title compound (109-4) (290 mg, 876 umol, 62.5% yield) as a white solid. LCMS (ES⁺): m/z 331.1 [M+H]⁺.

Synthesis of N-(1-(5-chloro-2-((5-(2-oxopyrrolidin-1-yl)pyridin-3-yl)amino)pyrimidin-4-yl)piperidin-3-yl)pentanamide (Compound 110). To a solution of N-(1-(2,5-dichloropyrimidin-4-yl)piperidin-3-yl)pentanamide (109-4) (200 mg, 604 umol, 1.00 eq) in dioxane (6 mL) were added 1-(5-amino-3-pyridyl)pyrrolidin-2-one (153 mg, 604 umol, 70.0% purity, 1.00 eq), Cs₂CO₃ (393 mg, 1.21 mmol, 2.00 eq), Pd(OAc)₂ (6.78 mg, 30.2 umol, 0.0500 eq) and Xantphos (10.5 mg, 18.1 umol, 0.0300 eq) at 25° C. under N₂ atmosphere. The mixture was stirred at 100° C. for 12 h. LCMS showed N-(1-(2,5-dichloropyrimidin-4-yl)piperidin-3-yl)pentanamide (109-4) was consumed and the desired mass was detected. The reaction mixture was concentrated under reduced pressure to remove solvent. The residue was purified by prep-HPLC (column: Phenomenex Luna C18 75*30 mm*3 um; mobile phase: [water (FA)-ACN]; B %: 15%-40%, 8 min) to give the title compound (Compound 110) (76.2 mg, 160 umol, 26.4% yield, 99.0% purity) as a white solid. LCMS (ES⁺): m/z 472.3 [M+H]⁺.

### Example 110. Synthesis of 1-(5-((5-chloro-4-(2-isopropylpyridin-4-yl)pyrimidin-2-yl)amino)pyridin-3-yl)pyrrolidin-2-one (Compound 111)

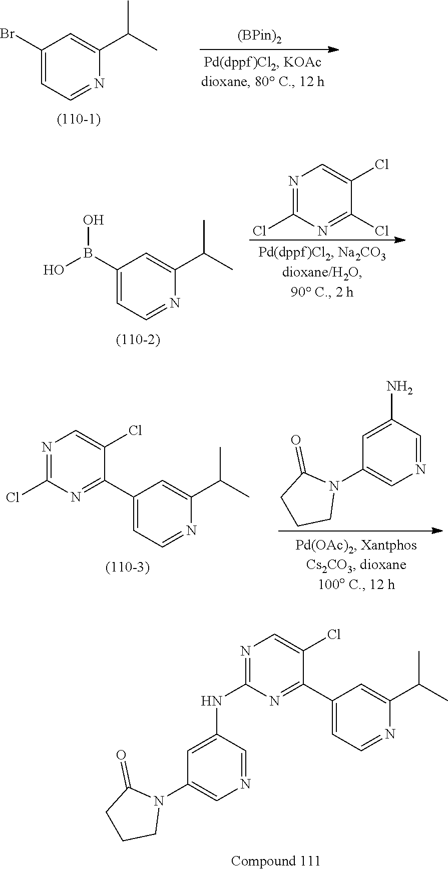

Synthesis of (2-isopropylpyridin-4-yl)boronic acid (110-2). To a solution of 4-bromo-2-isopropylpyridine (110-1) (900 mg, 4.50 mmol, 1.00 eq) in dioxane (20 mL) were added 4,4,5,5-tetramethyl-2-(4,4,5,5-tetramethyl-1,3,2-dioxaborolan-2-yl)-1,3,2-dioxaborolane (1.37 g, 5.40 mmol, 1.20 eq), KOAc (883 mg, 9.00 mmol, 2.00 eq) and Pd(dppf)Cl₂ (165 mg, 225 μmol, 0.0500 eq) at 25° C. under N₂ atmosphere. The mixture was stirred at 80° C. for 12 h. LCMS showed 4-bromo-2-isopropylpyridine (110-1) was consumed and the desired mass was detected. Water (10 mL) was added to the mixture at 25° C. Then the reaction mixture was extracted with EtOAc 15 mL. The organic layers were dried over anhydrous sodium sulfate, filtered and the filtrate was concentrated under reduced pressure to give a residue. The residue was purified by prep-HPLC (column: Phenomenex Luna C18 200*40 mm*10 um; mobile phase: [water (FA)-ACN]; B %: 1%-13%, 5 min) to give the title compound (110-2) as a white solid. LCMS (ES⁺): m/z 166.2 [M+H]⁺.

Synthesis of 2,5-dichloro-4-(2-isopropylpyridin-4-yl)pyrimidine (110-3). To a solution of (2-isopropylpyridin-4-yl)boronic acid (110-2) (200 mg, 1.21 mmol, 1.00 eq) in dioxane (10 mL) and H₂O (1 mL) were added 2,4,5-trichloropyrimidine (222 mg, 1.21 mmol, 1.00 eq), Na₂CO₃ (385 mg, 3.64 mmol, 121 μL, 3.00 eq) and Pd(PPh₃)₄ (42.0 mg, 36.4 μmol, 0.0300 eq) at 25° C. under N₂ atmosphere. The mixture was stirred at 90° C. for 2 h. LCMS showed (2-isopropylpyridin-4-yl)boronic acid (110-2) was consumed and the desired mass was detected. Water (10 mL) was added to the mixture at 25° C. Then the reaction mixture was extracted with EtOAc 15 mL. The organic layers were dried over anhydrous sodium sulfate, filtered and the filtrate was concentrated under reduced pressure to give a residue. The residue was purified by prep-TLC (SiO₂, Petroleum ether:Ethyl acetate=3:1) to give the title compound (110-3) (300 mg, 1.12 mmol, 92.3% yield) as a colorless oil. LCMS (ES⁺): m/z 268.1 [M+H]⁺.

Synthesis of 1-(5-((5-chloro-4-(2-isopropylpyridin-4-yl)pyrimidin-2-yl)amino)pyridin-3-yl)pyrrolidin-2-onem (Compound 111). To a solution of 1-(5-amino-3-pyridyl)pyrrolidin-2-one (189 mg, 746 μmol, 70% purity, 1.00 eq) in dioxane (6 mL) were added 2,5-dichloro-4-(2-isopropylpyridin-4-yl)pyrimidine (110-3) (200 mg, 746 μmol, 1.00 eq), Pd(OAc)₂ (8.37 mg, 37.3 μmol, 0.0500 eq), Cs₂CO₃ (486 mg, 1.49 mmol, 2.00 eq) and Xantphos (13.0 mg, 22.4 μmol, 0.0300 eq) at 25° C. under N₂ atmosphere. The mixture was stirred at 100° C. for 12 h. LCMS showed 2,5-dichloro-4-(2-isopropylpyridin-4-yl)pyrimidine (110-3) was consumed and the desired mass was detected. The reaction mixture was concentrated under reduced pressure to remove solvent. The residue was purified by prep-HPLC (column: Phenomenex luna C18 80*40 mm*3 um; mobile phase: [water (FA)-ACN]; B %: 1%-35%, 8 min) to give the title compound (Compound 111) (76.0 mg, 184 μmol, 24.7% yield, 99.0% purity) as a white solid. LCMS (ES⁺): m/z 409.1 [M+H]⁺.

### Example 111. Synthesis of 1-(5-((5-chloro-4-(2-chloro-4-fluorophenyl)pyrimidin-2-yl)amino)pyridin-3-yl)pyrrolidin-2-one (Compound 112)

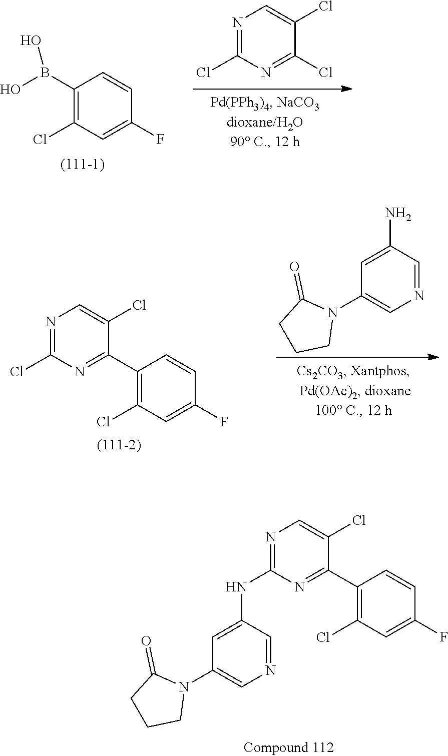 [SMILES: OB(O)c1ccc(F)cc1Cl ; Clc1ncc(Cl)c(Cl)n1 ; Fc1ccc(-c2nc(Cl)ncc2Cl)c(Cl)c1 ; Nc1cncc(N2CCCC2=O)c1 ; O=C1CCCN1c1cncc(Nc2ncc(Cl)c(-c3ccc(F)cc3Cl)n2)c1]

Synthesis of 2,5-dichloro-4-(2-chloro-4-fluorophenyl)pyrimidine (111-2). To a solution of 2,4,5-trichloropyrimidine (300 mg, 1.72 mmol, 1.00 eq) in dioxane (6 mL) and H₂O (0.6 mL) were added Pd(PPh₃)₄ (39.8 mg, 34.4 μmol, 0.0200 eq), Na₂CO₃ (365 mg, 3.44 mmol, 2.00 eq) and (2-chloro-4-fluorophenyl)boronic acid (111-1) (631 mg, 3.44 mmol, 2.00 eq) at 25° C. under N₂. The mixture was stirred at 90° C. for 12 h under N₂ atmosphere. LCMS showed that the desired mass was detected. Water (5 mL) was added to the mixture at 25° C. Then the reaction mixture was extracted with EtOAc (6 mL*3). The combined organic layers were dried over Na₂SO₄, filtered and the filtrate was concentrated under reduced pressure to give a residue. The residue was purified by prep-TLC (SiO₂, Petroleum ether:Ethyl acetate=5:1) to give the title compound (111-2) (450 mg, crude) as a colorless oil. LCMS (ES⁺): m/z 277.0 [M+H]⁺.

Synthesis of 1-(5-((5-chloro-4-(2-chloro-4-fluorophenyl)pyrimidin-2-yl)amino)pyridin-3-yl)pyrrolidin-2-one (Compound 112). To a solution of 2,5-dichloro-4-(2-chloro-4-fluorophenyl)pyrimidine (111-2) (250 mg, 901 μmol, 0.800 eq) in dioxane (10 mL) were added Cs₂CO₃ (734 mg, 2.25 mmol, 2.00 eq), Xantphos (19.6 mg, 33.8 μmol, 0.0300 eq), Pd(OAc)₂ (12.6 mg, 56.3 μmol, 0.0500 eq) and 1-(5-amino-3-pyridyl)pyrrolidin-2-one (314 mg, 1.24 mmol, 70.0% purity, 1.10 eq) at 25° C. under N₂. The mixture was stirred at 100° C. for 12 h under N₂ atmosphere. LCMS showed that 2,5-dichloro-4-(2-chloro-4-fluorophenyl)pyrimidine (111-2) was consumed and the desired mass was detected. Water (5 mL) was added to the mixture at 25° C. Then the reaction mixture was extracted with EtOAc (6 mL*3). The organic layers were dried over Na₂SO₄, filtered and the filtrate was concentrated under reduced pressure to give a residue. The residue was purified by prep-HPLC (column: Phenomenex luna C18 80*40 mm*3 um; mobile phase: [water (FA)-ACN]; B %: 25%-65%, 8 min) to give the title compound (Compound 112) (56.3 mg, 129 μmol, 11.5% yield, 96.1% purity) as a white solid. LCMS (ES⁺): m/z 417.9 [M+H]⁺.

### Example 112. Synthesis of 1-(5-((5-chloro-4-(4-fluoro-2methylphenyl)pyrimidin-2-yl)amino)pyridin-3-yl)pyrrolidin-2-one (Compound 113)

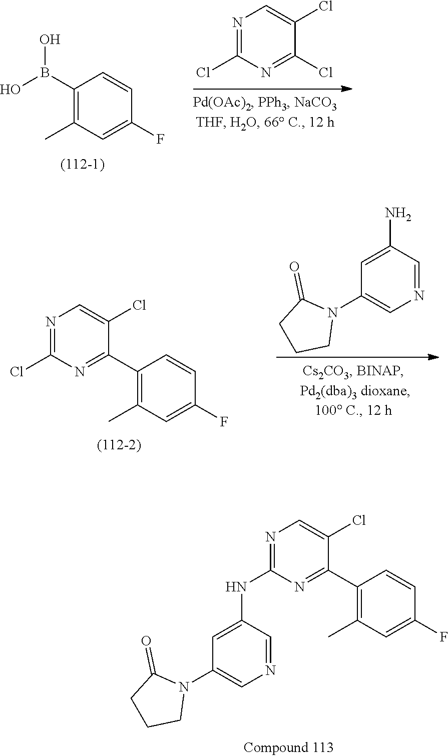 [SMILES: Cc1cc(F)ccc1B(O)O ; Clc1ncc(Cl)c(Cl)n1 ; Cc1cc(F)ccc1-c1nc(Cl)ncc1Cl ; Nc1cncc(N2CCCC2=O)c1 ; Cc1cc(F)ccc1-c1nc(Nc2cncc(N3CCCC3=O)c2)ncc1Cl]

Synthesis of 2,5-dichloro-4-(4-fluoro-2-methylphenyl)pyrimidine (112-2). To a solution of (4-fluoro-2-methylphenyl)boronic acid (112-1) (441 mg, 2.86 mmol, 1.05 eq) in THE (10 mL) were added 2,4,5-trichloropyrimidine (500 mg, 2.73 mmol, 1.00 eq), aq. Na₂CO₃ (1.00 M, 5.45 mL, 2.00 eq), PPh₃ (28.6 mg, 109 μmol, 0.0400 eq) and Pd(OAc)₂ (12.2 mg, 54.5 μmol, 0.0200 eq) at 25° C. under N₂ atmosphere. The mixture was stirred at 66° C. for 12 h. LCMS showed (4-fluoro-2-methylphenyl)boronic acid (112-1) was consumed and the desired mass was detected. The reaction mixture was concentrated under reduced pressure to remove solvent. The residue was purified by prep-TLC (SiO₂, Petroleum ether:Ethyl acetate=20:1) to give the title compound (112-2) (500 mg, 1.94 mmol, 71.4% yield) as a colorless oil. LCMS (ES⁺): m/z 257.1 [M+H]⁺.

Synthesis of 1-(5-((5-chloro-4-(4-fluoro-2methylphenyl)pyrimidin-2-yl)amino)pyridin-3-yl)pyrrolidin-2-one (Compound 113). To a solution of 2,5-dichloro-4-(4-fluoro-2-methylphenyl)pyrimidine (112-2) (470 mg, 1.83 mmol, 1.00 eq) in dioxane (15 mL) were added 1-(5-amino-3-pyridyl)pyrrolidin-2-one (421 mg, 1.83 mmol, 77.0% purity, 1.00 eq), Cs₂CO₃ (1.31 g, 4.02 mmol, 2.20 eq), BINAP (79.7 mg, 128 μmol, 0.0700 eq) and Pd₂(dba)₃ (83.7 mg, 91.4 μmol, 0.0500 eq) at 25° C. under N₂ atmosphere. The mixture was stirred at 100° C. for 12 h. LCMS showed 2,5-dichloro-4-(4-fluoro-2-methylphenyl)pyrimidine (112-2) was consumed and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH4HCO3)-ACN]; B %: 40%-70%, 8 min) to give the title compound (Compound 113) (145 mg, 364 μmol, 19.9% yield, 100% purity) as a white solid. LCMS (ES⁺): m/z 398.1 [M+H]⁺.

### Example 113. Synthesis of 1-(5-((5-chloro-4-(2,6-dimethylmorpholino)pyrimidin-2-yl)amino)pyridin-3-yl)pyrrolidin-2-one (Compound 114)

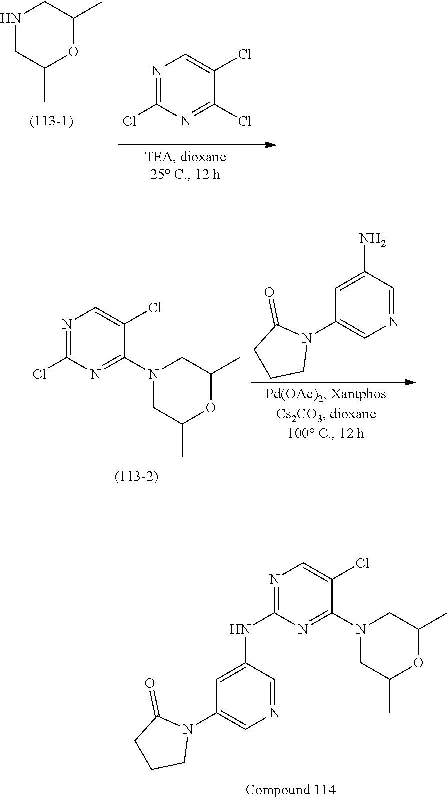

Synthesis of 4-(2,5-dichloropyrimidin-4-yl)-2,6-dimethylmorpholine (113-2). To a solution of 2,4,5-trichloropyrimidine (332 mg, 1.81 mmol, 1.00 eq) in dioxane (5 mL) were added TEA (201 mg, 1.99 mmol, 277 μL, 1.10 eq) and 2,6-dimethylmorpholine (113-1) (250 mg, 2.17 mmol, 267 μL, 1.20 eq) at 25° C. The mixture was stirred at 25° C. for 12 h. LCMS showed 2,6-dimethylmorpholine (113-1) was consumed and the desired mass was detected. The reaction mixture was concentrated under reduced pressure to remove solvent. The residue was purified by prep-TLC (SiO₂, Petroleum ether:Ethyl acetate=10:1) to give the title compound (113-2) (465 mg, 1.78 mmol, 98.1% yield) as a colorless oil. LCMS (ES⁺): m/z 262.1 [M+H]⁺.

Synthesis of 1-(5-((5-chloro-4-(2,6-dimethylmorpholino)pyrimidin-2-yl)amino)pyridin-3-yl)pyrrolidin-2-one (Compound 114). To a solution of 4-(2,5-dichloropyrimidin-4-yl)-2,6-dimethylmorpholine (113-2) (200 mg, 763 μmol, 1.00 eq) in dioxane (5 mL) were added 1-(5-amino-3-pyridyl)pyrrolidin-2-one (193 mg, 763 μmol, 70.0% purity, 1.00 eq), Cs₂CO₃ (497 mg, 1.53 mmol, 2.00 eq), Xantphos (13.2 mg, 22.9 μmol, 0.0300 eq) and Pd(OAc)₂ (8.56 mg, 38.1 μmol, 0.0500 eq) at 25° C. under N₂ atmosphere. The mixture was stirred at 100° C. for 12 h. LCMS showed 4-(2,5-dichloropyrimidin-4-yl)-2,6-dimethylmorpholine (113-2) was consumed and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH4HCO3)-ACN]; B %: 40%-65%, 8 min) to give the title compound (Compound 114) (26.7 mg, 66.3 μmol, 8.69% yield, 100% purity) as a yellow solid. LCMS (ES⁺): m/z 403.1 [M+H]⁺.

### Example 114. Synthesis of 1-(5-((5-chloro-4-(3,5-dimethylpiperidin-1-yl)pyrimidin-2-yl)amino)pyridin-3-yl)pyrrolidin-2-one (Compound 115)

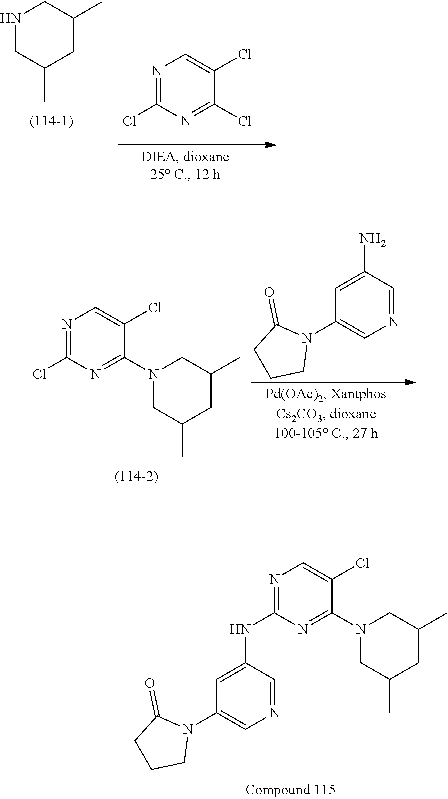

Synthesis of 2,5-dichloro-4-(3,5-dimethylpiperidin-1-yl)pyrimidine (114-2). To a solution of 3,5-dimethylpiperidine (114-1) (300 mg, 2.65 mmol, 352 μL, 1.20 eq) in dioxane (6 mL) were added DIEA (314 mg, 2.43 mmol, 423 μL, 1.10 eq) and 2,4,5-trichloropyrimidine (405 mg, 2.21 mmol, 1.00 eq) at 25° C. The mixture was stirred at 25° C. for 12 h. LCMS showed 3,5-dimethylpiperidine 114-1 was consumed and the desired mass was detected. The reaction mixture was concentrated under reduced pressure to remove solvent. The residue was purified by prep-TLC (SiO₂, Ethyl acetate:Petroleum ether=5:1) to give the title compound (114-2) (360 mg, 1.38 mmol, 62.7% yield) as a white solid. LCMS (ES⁺): m/z 260.1 [M+H]⁺.

Synthesis of 1-(5-((5-chloro-4-(3,5-dimethylpiperidin-1-yl)pyrimidin-2-yl)amino)pyridin-3-yl)pyrrolidin-2-one (Compound 115). To a solution of 2,5-dichloro-4-(3,5-dimethylpiperidin-1-yl)pyrimidine (114-2) (350 mg, 1.38 mmol, 70.0% purity, 1.00 eq) in dioxane (5 mL) were added 2,5-dichloro-4-(3,5-dimethyl-1-piperidyl)pyrimidine (360 mg, 1.38 mmol, 1.00 eq), Cs₂CO₃ (902 mg, 2.77 mmol, 2.00 eq), Xantphos (24.0 mg, 41.5 μmol, 0.0300 eq) and Pd(OAc)₂ (15.5 mg, 69.2 μmol, 0.0500 eq) at 25° C. under N₂ atmosphere. The mixture was stirred at 100° C. for 12 h. LCMS showed ˜52% of 2,5-dichloro-4-(3,5-dimethylpiperidin-1-yl)pyrimidine (114-2) was remaining and the desired mass was detected. Then to the mixture were added Cs₂CO₃ (902 mg, 2.77 mmol, 2.00 eq), Xantphos (24.0 mg, 41.5 μmol, 0.0300 eq) and Pd(OAc)₂ (15.5 mg, 69.2 μmol, 0.0500 eq) at 25° C. under N₂ atmosphere. The mixture was stirred at 100° C. for 3 h and LCMS showed ˜45% of 2,5-dichloro-4-(3,5-dimethylpiperidin-1-yl)pyrimidine (114-2) was remaining and the desired mass was detected. Then the mixture was stirred at 105° C. for 12 h. LC-MS showed ˜15% of 2,5-dichloro-4-(3,5-dimethylpiperidin-1-yl)pyrimidine (114-2) was remaining and the desired mass was detected. The reaction mixture was concentrated under reduced pressure to remove solvent. The residue was purified by prep-HPLC (column: Phenomenex luna C18 100*40 mm*3 um; mobile phase: [water (FA)-ACN]; B %: 30%-70%, 8 min) to give the title compound (Compound 115) (18.2 mg, 45.4 μmol, 3.28% yield, 100% purity) as a white solid. LCMS (ES⁺): m/z 401.3 [M+H]⁺.

### Example 115. Synthesis of 1-(5-((5-chloro-4-(o-tolyl)pyrimidin-2-yl)amino)pyridin-3-yl)pyrrolidin-2-one (Compound 116)

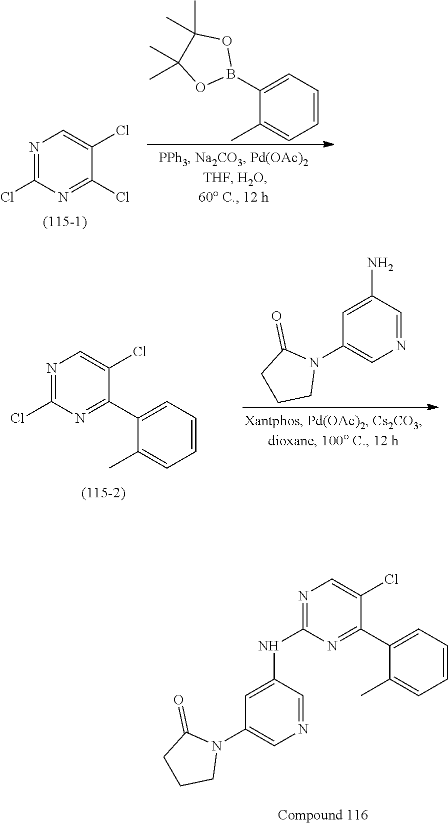 [SMILES: Clc1ncc(Cl)c(Cl)n1 ; Cc1ccccc1B1OC(C)(C)C(C)(C)O1 ; Cc1ccccc1-c1nc(Cl)ncc1Cl ; Nc1cncc(N2CCCC2=O)c1 ; Cc1ccccc1-c1nc(Nc2cncc(N3CCCC3=O)c2)ncc1Cl]

Synthesis of 2,5-dichloro-4-(o-tolyl)pyrimidine (115-2). To a stirred solution of 2,4,5-trichloropyrimidine (115-1) (1.00 g, 5.45 mmol, 1.00 eq) and 4,4,5,5-tetramethyl-2-(o-tolyl)-1,3,2-dioxaborolane (1.19 g, 5.45 mmol, 1.00 eq) in THF (2.5 mL) and water (0.5 mL) were added PPh₃ (71.5 mg, 273 μmol, 0.0500 eq), Pd(OAc)₂ (61.2 mg, 273 μmol, 0.0500 eq) and Na₂CO₃ (1.16 g, 10.9 mmol, 2.00 eq). The mixture was stirred at 60° C. for 12 h under N₂. LCMS showed the desired compound was detected. The reaction mixture was concentrated. The residue was purified by column chromatography (SiO₂, Petroleum ether:Ethyl acetate=10:1 to 1:1) to give the title compound (115-2) (1.10 g, 2.72 mmol, 49.8% yield, 59.0% purity) as a white solid. LCMS (ES⁺): m/z 239.0 [M+H]⁺.

Synthesis of 1-(5-((5-chloro-4-(o-tolyl)pyrimidin-2-yl)amino)pyridin-3-yl)pyrrolidin-2-one (Compound 116). To a stirred solution of 2,5-dichloro-4-(o-tolyl)pyrimidine (115-2) (200 mg, 836 μmol, 1.00 eq) and 1-(5-amino-3-pyridyl)pyrrolidin-2-one (163 mg, 920 μmol, 1.10 eq) in dioxane (6 mL) were added Cs₂CO₃(681 mg, 2.09 mmol, 2.50 eq), Pd(OAc)₂ (18.8 mg, 83.7 μmol, 0.100 eq) and Xantphos (96.8 mg, 167 μmol, 0.200 eq). The mixture was stirred at 100° C. for 12 h under N₂. LCMS showed the desired compound was detected. The reaction mixture was concentrated. The residue was purified by prep-HPLC (column: Waters Xbridge Prep OBD C18 150*40 mm*10 um; mobile phase: [water (NH4HCO3)-ACN]; B %: 30%-50%, 8 min) to give the title compound (Compound 116) (7.60 mg, 20.0 μmol, 2.39% yield, 100.00% purity) as a white solid. LCMS (ES⁺): m/z 380.1 [M+H]⁺.

### Example 116. Synthesis of 1-(5-((5-chloro-4-phenylpyridin-2-yl)amino)pyridin-3-yl)pyrrolidin-2-one (Compound 117)

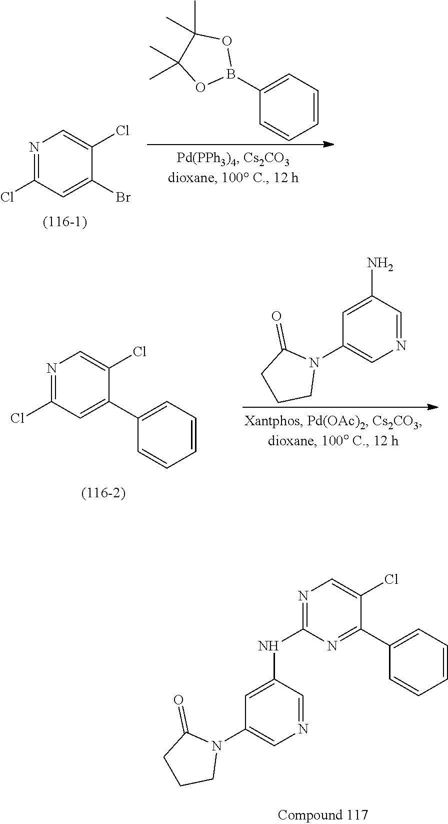 [SMILES: Clc1cc(Br)c(Cl)cn1 ; CC1(C)OB(c2ccccc2)OC1(C)C ; Clc1cc(-c2ccccc2)c(Cl)cn1 ; Nc1cncc(N2CCCC2=O)c1 ; O=C1CCCN1c1cncc(Nc2ncc(Cl)c(-c3ccccc3)n2)c1]

Synthesis of 2,5-dichloro-4-phenylpyridine (116-2). To a solution of 4-bromo-2,5-dichloropyridine (116-1) (1.00 g, 4.41 mmol, 1.00 eq), 4,4,5,5-tetramethyl-2-phenyl-1,3,2-dioxaborolane (899 mg, 4.41 mmol, 1.00 eq) and Cs₂CO₃(3.59 g, 11.0 mmol, 2.50 eq) in dioxane (10 mL) was added Pd(PPh₃)₄ (204 mg, 176 μmol, 0.0400 eq). The mixture was stirred at 100° C. for 12 h under N₂. LCMS showed 4-bromo-2, 5-dichloropyridine (116-1) was consumed and desired mass was detected. The reaction mixture was concentrated under reduced pressure to get a residue. The reaction mixture was partitioned between H₂O (10 mL) and EtOAc (10 mL). The organic phase was separated, dried over Na₂SO₄, filtered and concentrated under reduced pressure to give the title compound (116-2) (900 mg, 4.02 mmol, 91.1% yield) as a yellow solid. LCMS (ES⁺): m/z 223.9 [M+H]⁺.

Synthesis of 1-(5-((5-chloro-4-phenylpyridin-2-yl)amino)pyridin-3-yl)pyrrolidin-2-one (Compound 117). To a solution of 2,5-dichloro-4-phenylpyridine (116-2) (300 mg, 1.34 mmol, 1.00 eq), 1-(5-amino-3-pyridyl)pyrrolidin-2-one (261 mg, 1.47 mmol, 1.10 eq), Cs₂CO₃ (1.09 g, 3.35 mmol, 2.50 eq) and Xantphos (155 mg, 268 μmol, 0.200 eq) in dioxane (8 mL) was added Pd(OAc)₂ (30.06 mg, 133.88 μmol, 0.1 eq). The mixture was stirred at 100° C. for 12 h under N₂. LCMS showed 2, 5-dichloro-4-phenylpyridine (116-2) was consumed and desired mass was detected. The reaction mixture was concentrated. The residue was purified by prep-HPLC (column: Phenomenex luna C18 80*40 mm*3 um; mobile phase: [water (FA)-ACN]; B %: 15%-55%, 8 min) to give the title compound (Compound 117) (105 mg, 288 μmol, 21.5% yield, 100.00% purity) as a white solid. LCMS (ES⁺): m/z 365.0 [M+H]⁺.

### Example 117. Synthesis of 1-(5-((5-chloro-4-(4-fluorophenyl)pyrimidin-2-yl)amino)pyridin-3-yl)pyrrolidin-2-one (Compound 118)

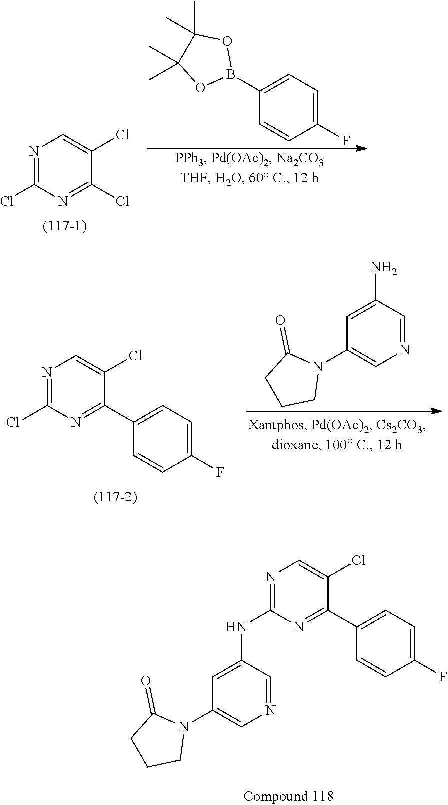 [SMILES: Clc1ncc(Cl)c(Cl)n1 ; CC1(C)OB(c2ccc(F)cc2)OC1(C)C ; Fc1ccc(-c2nc(Cl)ncc2Cl)cc1 ; Nc1cncc(N2CCCC2=O)c1 ; O=C1CCCN1c1cncc(Nc2ncc(Cl)c(-c3ccc(F)cc3)n2)c1]

Synthesis of 2,5-dichloro-4-(4-fluorophenyl)pyrimidine (117-2). To a solution of 2,4,5-trichloropyrimidine (117-1) (2.00 g, 10.9 mmol, 1.00 eq) and 2-(4-fluorophenyl)-4,4,5,5-tetramethyl-1,3,2-dioxaborolane (2.42 g, 10.9 mmol, 1.00 eq) in THF (20 mL) and H₂O (4 mL) were added Na₂CO₃ (2.31 g, 21.8 mmol, 2.00 eq), PPh₃ (143 mg, 545 μmol, 0.0500 eq) and Pd(OAc)₂ (122 mg, 545 μmol, 0.0500 eq). The mixture was stirred at 60° C. for 12 h under N₂. LCMS showed desired MS was detected. The reaction mixture was diluted with H₂O (10 mL) and extracted with ethyl acetate (10 mL*3). The combined organic layers were dried over Na₂SO₄, filtered and concentrated under reduced pressure to give a residue. The residue was purified by column chromatography (SiO₂, Petroleum ether:Ethyl acetate=20:1 to 10:1) to give the title compound (117-2) (2.40 g, crude) as a white solid. LCMS (ES⁺): m/z 242.9 [M+H]⁺.

Synthesis of 1-(5-((5-chloro-4-(4-fluorophenyl)pyrimidin-2-yl)amino)pyridin-3-yl)pyrrolidin-2-one (Compound 118). To a solution of 2,5-dichloro-4-(4-fluorophenyl)pyrimidine (117-2) (100 mg, 411 μmol, 1.00 eq) and 1-(5-amino-3-pyridyl)pyrrolidin-2-one (80.2 mg, 453 μmol, 1.10 eq) in dioxane (10 mL) were added Cs₂CO₃ (268 mg, 823 μmol, 2.00 eq), Pd(OAc)₂ (4.62 mg, 20.6 μmol, 0.0500 eq) and Xantphos (23.8 mg, 41.1 μmol, 0.100 eq). The mixture was stirred at 100° C. for 12 h under N₂. LCMS showed desired MS was detected. The reaction mixture was concentrated under reduced pressure to give a residue. The residue was purified by prep-HPLC (column: Phenomenex luna C18 80*40 mm*3 um; mobile phase: [water (FA)-ACN]; B %: 45%-75%, 8 min) to give the title compound (Compound 118) (5.00 mg, 13.0 μmol, 3.17% yield, 100.00% purity) as a white solid. LCMS (ES⁺): m/z 383.9 [M+H]⁺.

### Example 118. Synthesis of 1-(5-((5-chloro-4-(2-(trifluoromethyl)piperidin-1-yl)pyrimidin-2-yl)amino)pyridin-3-yl)pyrrolidin-2-one (Compound 119)

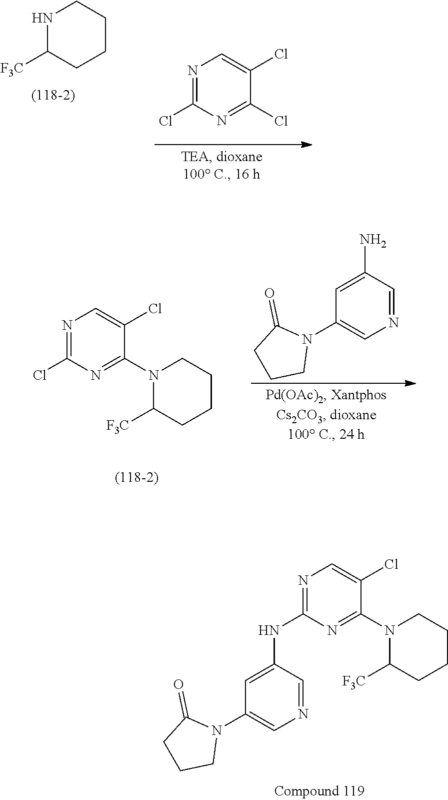 [SMILES: FC(F)(F)C1CCCCN1 ; Clc1ncc(Cl)c(Cl)n1 ; FC(F)(F)C1CCCCN1c1nc(Cl)ncc1Cl ; Nc1cncc(N2CCCC2=O)c1 ; O=C1CCCN1c1cncc(Nc2ncc(Cl)c(N3CCCCC3C(F)(F)F)n2)c1]

Synthesis of 2,5-dichloro-4-(2-(trifluoromethyl)piperidin-1-yl)pyrimidine (118-2). To a solution of 2-(trifluoromethyl)piperidine (118-1) (250 mg, 1.63 mmol, 1.20 eq) in dioxane (10 mL) were added TEA (151 mg, 1.50 mmol, 208 μL, 1.10 eq) and 2,4,5-trichloropyrimidine (250 mg, 1.36 mmol, 1.00 eq) at 25° C. The mixture was stirred at 100° C. for 12 h. LCMS showed ˜77.9% of 2-(trifluoromethyl)piperidine (118-1) was remaining and the desired mass was detected. Then the mixture was stirred at 100° C. for 4 h. LC-MS showed ˜74.0% of 2-(trifluoromethyl)piperidine (118-1) was remaining and the desired mass was detected. The reaction mixture was concentrated under reduced pressure to remove solvent. The residue was purified by prep-TLC (SiO₂, Petroleum ether:Ethyl acetate=10:1) to give the title compound (118-2) (70.0 mg, 233 μmol, 17.2% yield) as a white solid. LCMS (ES⁺): m/z 300.1 [M+H]⁺.

Synthesis of 1-(5-((5-chloro-4-(2-(trifluoromethyl)piperidin-1-yl)pyrimidin-2-yl)amino)pyridin-3-yl)pyrrolidin-2-one (Compound 119). To a solution of 2,5-dichloro-4-(2-(trifluoromethyl)piperidin-1-yl)pyrimidine (118-2) (70.0 mg, 233 μmol, 1.00 eq) in dioxane (5 mL) were added 1-(5-amino-3-pyridyl)pyrrolidin-2-one (59.1 mg, 233 μmol, 70.0% purity, 1.00 eq), Xantphos (4.05 mg, 7.00 μmol, 0.0300 eq), Cs₂CO₃ (152 mg, 467 μmol, 2.00 eq) and Pd(OAc)₂ (2.62 mg, 11.7 μmol, 0.0500 eq) at 25° C. under N₂ atmosphere. The mixture was stirred at 100° C. for 12 h. LCMS showed ˜31.1% of 2,5-dichloro-4-(2-(trifluoromethyl)piperidin-1-yl)pyrimidine (118-2) was remaining and the desired mass was detected. Then Xantphos (4.05 mg, 7.00 μmol, 0.0300 eq), Cs₂CO₃ (152 mg, 467 μmol, 2.00 eq) and Pd(OAc)₂ (2.62 mg, 11.7 μmol, 0.0500 eq) were added to the mixture and then stirred at 100° C. for another 12 h. LCMS showed 2,5-dichloro-4-(2-(trifluoromethyl)piperidin-1-yl)pyrimidine (118-2) was consumed and the desired mass was detected. The reaction mixture was concentrated under reduced pressure to remove solvent. The residue was purified by prep-HPLC (column: Phenomenex Luna C18 75*30 mm*3 um; mobile phase: [water (FA)-ACN]; B %: 15%-55%, 8 min). But the 1H NMR was messy, then the crude product was purified by prep-HPLC (column: Waters Xbridge BEH C18 100*30 mm*10 um; mobile phase: [water (NH4HCO3)-ACN]; B %: 40%-70%, 8 min) to give the title compound (Compound 119) (7.20 mg, 16.0 μmol, 6.85% yield, 98.0% purity) as a white solid. LCMS (ES⁺): m/z 441.1 [M+H]⁺.

### Example 119. Synthesis of 1-(5-((5-chloro-4-(3-(pyridin-2-yl)pyrrolidin-1-yl)pyrimidin-2-yl)amino)pyridin-3-yl)pyrrolidin-2-one (Compound 120)

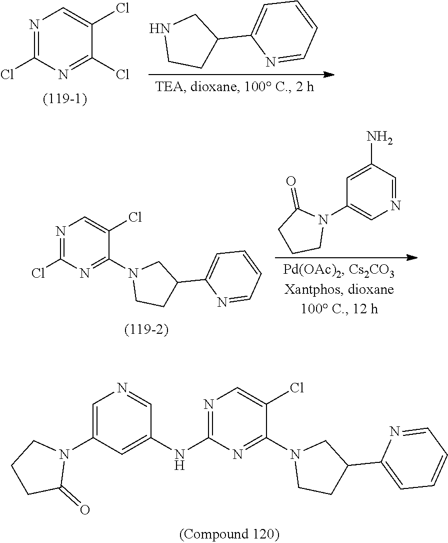

Synthesis of 2,5-dichloro-4-(3-(pyridin-2-yl)pyrrolidin-1-yl)pyrimidine (119-2). To a solution of 2,4,5-trichloropyrimidine (119-1) (297 mg, 1.62 mmol, 1.00 eq) and 2-pyrrolidin-3-ylpyridine (240 mg, 1.62 mmol, 1.00 eq) in dioxane (3 mL) was added TEA (492 mg, 4.86 mmol, 676 μL, 3.00 eq) at 25° C. The mixture was stirred at 100° C. for 2 h. LCMS showed that the desired compound was detected. The reaction mixture was concentrated under reduced pressure to give the title compound (119-2) (320 mg, crude) as a brown solid. LCMS (ES⁺): m/z 295.0 [M+H]⁺.

Synthesis of 1-(5-((5-chloro-4-(3-(pyridin-2-yl)pyrrolidin-1-yl)pyrimidin-2-yl)amino)pyridin-3-yl)pyrrolidin-2-one (Compound 120). To a solution of 2,5-dichloro-4-[3-(2-pyridyl)pyrrolidin-1-yl]pyrimidine (119-2) (100 mg, 339 umol, 0.800 eq), 1-(5-amino-3-pyridyl)pyrrolidin-2-one (75.0 mg, 423 umol, 1.00 eq) and Cs₂CO₃ (414 mg, 1.27 mmol, 3.00 eq) in dioxane (3 mL) were added Xantphos (49.0 mg, 84.7 umol, 0.200 eq) and Pd(OAc)₂ (9.51 mg, 42.4 umol, 0.100 eq) at 25° C. The mixture was stirred at 100° C. for 12 h under N₂ atmosphere. LCMS showed that 2,5-dichloro-4-[3-(2-pyridyl)pyrrolidin-1-yl]pyrimidine (119-2) was consumed completely and the desired mass was detected. The reaction mixture was concentrated under reduced pressure to give a residue. The residue was purified by prep-HPLC (column: Phenomenex Luna 80*30 mm*3 um; mobile phase: [water (TFA)-ACN]; B %: 5%-35%, 8 min) to give the title compound (Compound 120) (53.9 mg, 124 umol, 29.2% yield) as a brown solid. LCMS (ES⁺): m/z 436.2 [M+H]⁺.

### Example 120. Synthesis of 1-(5-((5-chloro-4-(3-(piperidin-1-yl)pyrrolidin-1-yl)pyrimidin-2-yl)amino)pyridin-3-yl)pyrrolidin-2-one (Compound 121)

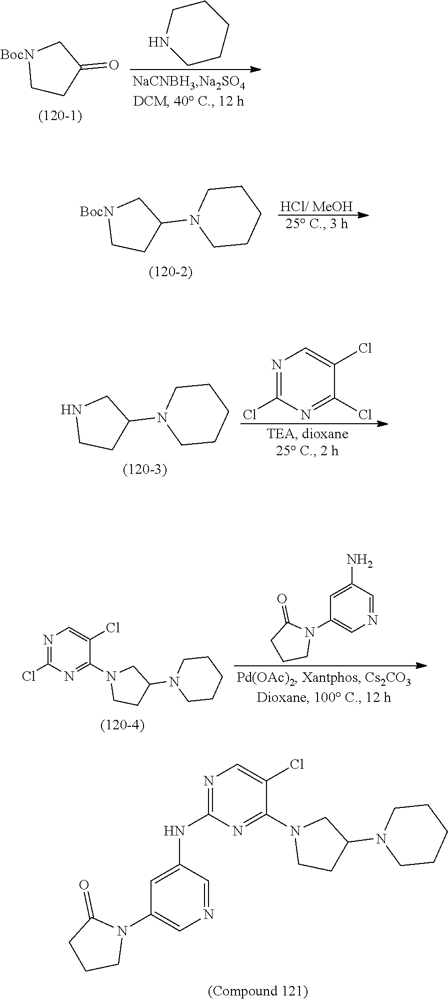

Synthesis of tert-butyl 3-(piperidin-1-yl)pyrrolidine-1-carboxylate (120-2). To a solution of piperidine (4.60 g, 54.0 mmol, 5.34 mL, 5.00 eq), NaBH₃CN (3.39 g, 54.0 mmol, 5 eq) and Na₂SO₄ (15.34 g, 107.98 mmol, 10.96 mL, 10 eq) in DCM (40 mL) was added tert-butyl 2-oxopyrrolidine-1-carboxylate (120-1) (2.00 g, 10.8 mmol, 1.83 mL, 1.00 eq) at 25° C. The mixture was stirred at 40° C. for 12 h. LCMS showed that the desired mass was detected. The reaction mixture was concentrated under reduced pressure. The residues was diluted with H₂O 50 mL and extracted with EtOAc (50 mL*3). The organic phase was separated, dried over Na₂SO₄, filtered and filtrate was concentrated under reduced pressure to give a residue. The residue was purified by flash silica gel chromatography (ISCO®; 20 g SepaFlash® Silica Flash Column, Eluent of 0-100% Ethyl acetate/Petroleum ether gradient @100 mL/min) to give the title compound (120-2) (2.00 g, 7.86 mmol, 72.8% yield) as a white solid. LCMS (ES⁺): m/z 255.1 [M+H]⁺

Synthesis of 1-(pyrrolidin-3-yl)piperidine (120-3). A solution of tert-butyl 3-(piperidin-1-yl)pyrrolidine-1-carboxylate (120-2) (1.40 g, 5.50 mmol, 1.00 eq) in HCl/MeOH (10 mL) was stirred at 25° C. for 3 h. LCMS showed that the desired mass was detected. The reaction mixture was concentrated under reduced pressure to give the title compound (120-3) (840 mg, 5.45 mmol, 98.9% yield) as a yellow solid. LCMS (ES⁺): m/z 155.0 [M+H]⁺

Synthesis of 2,5-dichloro-4-(3-(piperidin-1-yl)pyrrolidin-1-yl)pyrimidinef (120-4). To a solution of 2,4,5-trichloropyrimidine (1.52 g, 8.30 mmol, 1.60 eq) and TEA (1.57 g, 15.6 mmol, 2.17 mL, 3.00 eq) in dioxane (5 mL) was added 1-(pyrrolidin-3-yl)piperidine (120-3) (800 mg, 5.19 mmol, 1.00 eq) at 25° C. The mixture was stirred at 25° C. for 2 h. LCMS showed that the desired mass was detected. The reaction mixture was concentrated under reduced pressure to get a residue. The residue was purified by flash silica gel chromatography (ISCO®; 20 g SepaFlash® Silica Flash Column, Eluent of 0-100% Ethyl acetate/Petroleum ether gradient @100 mL/min) to give the title compound (120-4) (1.40 g, 3.90 mmol, 75.2% yield, 80.0% purity) as a white solid. LCMS (ES⁺): m/z 301.0 [M+H]⁺.

Synthesis of 1-(5-((5-chloro-4-(3-(piperidin-1-yl)pyrrolidin-1-yl)pyrimidin-2-yl)amino)pyridin-3-yl)pyrrolidin-2-one (Compound 121). To a solution of 2,5-dichloro-4-(3-(piperidin-1-yl)pyrrolidin-1-yl)pyrimidinef (120-4) (100 mg, 332 umol, 1.30 eq), 1-(5-amino-3-pyridyl)pyrrolidin-2-one (45.3 mg, 255 umol, 1.00 eq), Cs₂CO₃ (250 mg, 766 umol, 3.00 eq) and Xantphos (29.6 mg, 51.1 umol, 0.200 eq) in dioxane (2 mL) was added Pd(OAc)₂ (5.73 mg, 25.5 umol, 0.100 eq) at 25° C. The mixture was stirred at 100° C. for 12 h under N₂ atmosphere. LCMS showed that the desired mass was detected. The reaction mixture was filtered and the filtrate was concentrated under reduced pressure to remove solvent. The residue was purified by prep-HPLC (column: Phenomenex Luna C18 75*30 mm*3 um; mobile phase: [water (TFA)-ACN]; B %: 1%-30%, 8 min) to give the title compound (Compound 121) (24.5 mg, 50.1 umol, 19.6% yield, 90.4% purity) as a white solid. LCMS (ES⁺): m/z 442.2 [M+H]⁺.

### Example 121. 1-(5-((4-(3-(azetidin-1-yl)phenyl)-5-chloropyrimidin-2-yl)amino)pyridin-3-yl)pyrrolidin-2-one (Compound 122)

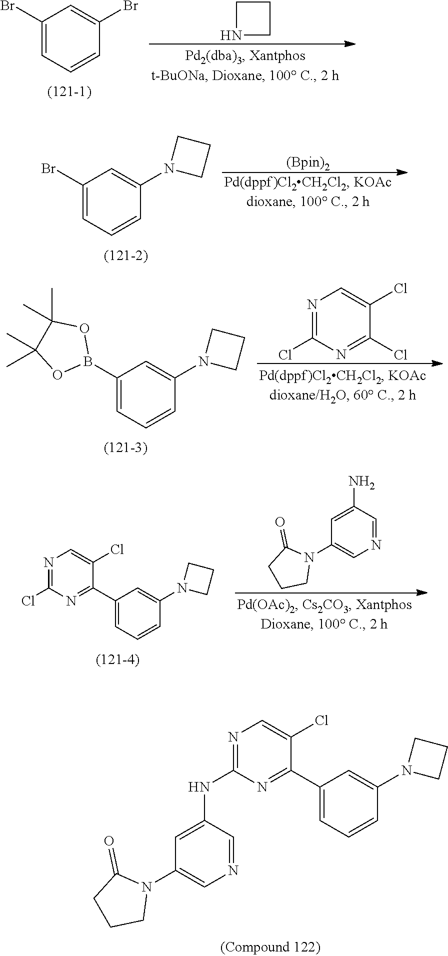

Synthesis of 1-(3-bromophenyl)azetidine (121-2). To a solution of 1,3-dibromobenzene (121-1) (12.4 g, 52.5 mmol, 6.32 mL, 1.50 eq) and azetidine (2.00 g, 35.0 mmol, 2.36 mL, 1.00 eq) in dioxane (20 mL) were added Pd₂(dba)₃ (1.60 g, 1.75 mmol, 0.0500 eq), Xantphos (2.03 g, 3.50 mmol, 0.100 eq) and t-BuONa (10.10 g, 105.09 mmol, 3 eq) at 25° C. The mixture was stirred at 100° C. for 2 h under N₂ atmosphere. LCMS showed the desired mass was detected. The reaction mixture was concentrated. The residue was purified by column chromatography (SiO₂, Petroleum ether/Ethyl acetate=10/1 to 1/1) to give the title compound (121-2) (2.90 g, 12.2 mmol, 34.7% yield, 88.9% purity) as a yellow oil. LCMS (ES⁺): m/z 212.0 [M+H]⁺.

Synthesis of 1-(3-(4,4,5,5-tetramethyl-1,3,2-dioxaborolan-2-yl)phenyl)azetidine (121-3). To a solution of 1-(3-bromophenyl)azetidine (121-2) (1.00 g, 4.72 mmol, 1.00 eq) and 4,4,5,5-tetramethyl-2-(4,4,5,5-tetramethyl-1,3,2-dioxaborolan-2-yl)-1,3,2-dioxaborolane (1.80 g, 7.07 mmol, 1.50 eq) in dioxane (10 mL) were added Pd(dppf)Cl₂·CH₂Cl₂ (385 mg, 472 umol, 0.100 eq), KOAc (1.39 g, 14.2 mmol, 3.00 eq) at 25° C. The mixture was stirred at 100° C. for 2 h under N₂ atmosphere. LCMS showed the desired mass was detected. The reaction mixture was concentrated. The residue was purified by column chromatography (SiO₂, Petroleum ether/Ethyl acetate=10/1 to 1/1) to give the title compound (121-3) (960 mg, 3.28 mmol, 69.5% yield, 88.5% purity) as a white solid. LCMS (ES⁺): m/z 260.2 [M+H]⁺.

Synthesis of 4-(3-(azetidin-1-yl)phenyl)-2,5-dichloropyrimidine (121-4). To a solution of 2,4,5-trichloropyrimidine (163 mg, 890 umol, 1.00 eq) and 1-(3-(4,4,5,5-tetramethyl-1,3,2-dioxaborolan-2-yl)phenyl)azetidine (121-3) (300 mg, 1.16 mmol, 1.30 eq) in dioxane (0.6 mL) and Water (0.2 mL) were added Pd(dppf)Cl₂·CH₂Cl₂ (50.9 mg, 62.3 umol, 0.0700 eq) and KOAc (262 mg, 2.67 mmol, 3.00 eq) at 25° C. The mixture was stirred at 60° C. for 2 h under N₂ atmosphere. LCMS showed the desired mass was detected. The reaction mixture was concentrated. The residue was purified by prep-TLC (SiO₂, Petroleum ether:Ethyl acetate=5:1) to give the title compound (121-4) (126 mg, 313 umol, 35.2% yield, 69.7% purity) as a yellow solid. LCMS (ES⁺): m/z 280.0 [M+H]⁺.

Synthesis of 1-(5-((4-(3-(azetidin-1-yl)phenyl)-5-chloropyrimidin-2-yl)amino)pyridin-3-yl)pyrrolidine-2-one (Compound 122). To a solution of 4-(3-(azetidin-1-yl)phenyl)-2,5-dichloropyrimidine (121-4) (100 mg, 357 umol, 1.00 eq) and 1-(5-amino-3-pyridyl)pyrrolidine-2-one (69.6 mg, 393 umol, 1.10 eq) in dioxane (2 mL) were added Xantphos (41.3 mg, 71.4 umol, 0.200 eq), Pd(OAc)₂ (8.01 mg, 35.7 umol, 0.100 eq) and Cs₂CO₃ (291 mg, 892 umol, 2.50 eq) at 25° C. The mixture was stirred at 100° C. for 2 h under N₂ atmosphere. LCMS showed the desired mass was detected. The reaction mixture was concentrated. The residue was purified by prep-HPLC (column: Waters Xbridge BEH C18 100*30 mm*10 um; mobile phase: [water (NH₄HCO₃)-ACN]; B %: 35%-75%, 8 min) to give the title compound (Compound 122) (5.80 mg, 12.3 umol, 3.44% yield, 89.1% purity) as a white solid. LCMS (ES⁺): m/z 421.2 [M+H]⁺.

### Example 122. Synthesis of 1-(5-((5-chloro-4-(3-cyclobutylpiperidin-1-yl)pyrimidin-2-yl)amino)pyridin-3-yl)pyrrolidin-2-one (Compound 123)

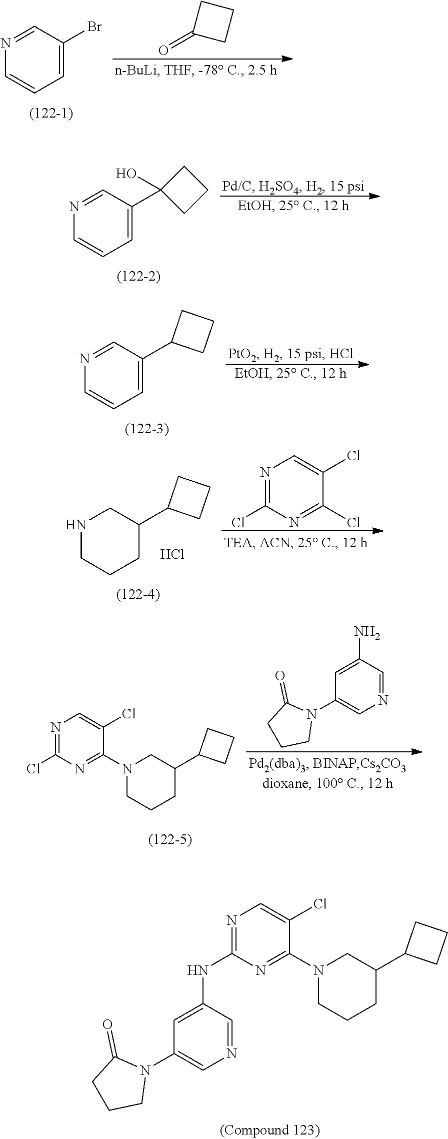

Synthesis of 1-(pyridin-3-yl)cyclobutan-1-ol (122-2). To a solution of 3-bromopyridine (122-1) (2.5 g, 15.8 mmol, 1.52 mL, 1.00 eq) in THF (50 mL) was added n-BuLi (2.5 M, 8.23 mL, 1.30 eq) and purged with N₂ at −78° C. for 3 times, the mixture was stirred at −78° C. for 0.5 h, then cyclobutanone (1.66 g, 23.7 mmol, 1.77 mL, 1.50 eq) was added to the mixture, the mixture was stirred at −78° C. for 2 hr under N₂ atmosphere. LC-MS showed 3-bromopyridine (122-1) was remained and desired mass was detected. The reaction mixture was quenched by addition H₂O 30 mL, and then diluted with EtOAc 30 mL and extracted with EtOAc (10 mL*3). The combined organic layers were washed with sat.aq. NaCl (10 mL), dried over Na₂SO₄, filtered and concentrated under reduced pressure. The residue was purified by column chromatography (SiO₂, Petroleum ether:Ethyl acetate=1:1, R_{f}=0.37). to give the title compound (122-2) (4.9 g, crude) as a yellow oil. LCMS (ES⁺): m/z 150.3 [M+H]⁺.

Synthesis of 3-cyclobutylpyridine (122-3). To a solution of 1-(pyridin-3-yl)cyclobutan-1-ol (122-2) (2 g, 13.4 mmol, 1.00 eq) in EtOH (10 mL) was added H₂SO₄ (1.58 g, 16.1 mmol, 857 μL, 1.20 eq) and Pd/C 10% (0.5 g, 1.34 mmol, 50% purity, 0.100 eq) at 0° C. under N₂ atmosphere. The suspension was degassed and purged with H₂ for 3 times. The mixture was stirred under H₂ (15 Psi) at 25° C. for 12 h. LC-MS showed 1-(pyridin-3-yl)cyclobutan-1-ol (122-2) was remained and the desired mass was detected. The reaction mixture was filtered and the filtrate concentrated under high vacuum. The residue was purified by column chromatography (SiO₂, Dichloromethane:Methanol=3:1, R_{f}=0.66) to give the title compound (122-3) (2 g, crude) was obtained as a yellow oil. LCMS (ES⁺): m/z 134.3 [M+H]⁺

Synthesis of 3-cyclobutylpiperidine (122-4). To a solution of 3-cyclobutylpyridine (122-3) (1.12 g, 8.39 mmol, 1.00 eq) in EtOH (30 mL) were added HCL (12 M, 1.05 mL, 1.50 eq) and PtO₂ (190 mg, 839 umol, 0.100 eq) under N₂ atmosphere. The suspension was degassed and purged with H₂ for 3 times. The mixture was stirred under H₂ (15 Psi) at 25° C. for 12 h. LC-MS showed 3-cyclobutylpyridine (122-3) was consumed completely and the desired mass was detected. The reaction mixture was filtered and the filtrate concentrated under high vacuum to give the title compound (122-4) (1 g, crude, HCl) as a yellow solid. LCMS (ES⁺): m/z 140.2 [M+H]⁺

Synthesis of 2,5-dichloro-4-(3-cyclobutylpiperidin-1-yl)pyrimidine (122-5). To a solution of 3-cyclobutylpiperidine (122-4) (450 mg, 2.45 mmol, 1.00 eq) in ACN (20 mL) was added TEA (744 mg, 7.36 mmol, 1.02 mL, 3.00 eq) and 2,4,5-trichloropyrimidine (512 mg, 3.68 mmol, 1.50 eq) at 25° C. The mixture was stirred at 25° C. for 12 h. LC-MS showed 3-cyclobutylpiperidine (122-4) was consumed completely and the desired mass was detected. The reaction mixture was filtered and the filtrate concentrated under high vacuum. The residue was purified by column chromatography (SiO₂, Petroleum ether:Ethyl acetate=3:1, R_{f}=0.68) to give the title compound (122-5) (530 mg, crude) as a yellow oil. LCMS (ES⁺): m/z 286.0 [M+H]⁺

Synthesis of 1-(5-((5-chloro-4-(3-cyclobutylpiperidin-1-yl)pyrimidin-2-yl)amino)pyridin-3-yl)pyrrolidin-2-one (Compound 123). To a solution of 2,5-dichloro-4-(3-cyclobutylpiperidin-1-yl)pyrimidine (122-5) (200 mg, 698 umol, 1.00 eq), 1-(5-aminopyridin-3-yl)pyrrolidin-2-one (185 mg, 1.05 mmol, 1.50 eq), Cs₂CO₃ (683 mg, 2.10 mmol, 3.00 eq), Pd₂(dba)₃ (63.9 mg, 69.8 umol, 0.100 eq) and BINAP (43.5 mg, 69.8 umol, 0.100 eq) in 1,4-dioxane (3 mL) was degassed at 25° C. and purged with N₂ for 3 times, and then the mixture was stirred at 100° C. for 12 h under N₂ atmosphere. LC-MS showed 2,5-dichloro-4-(3-cyclobutylpiperidin-1-yl)pyrimidine (122-5) was consumed completely and the desired mass was detected. The reaction mixture was quenched by addition H₂O 5 mL, and extracted with EtOAc (5 mL*3). The combined organic layers were washed with sat.aq. NaCl (2 mL), dried over Na₂SO₄, filtered and concentrated under reduced pressure. The residue was purified by prep-HPLC (Phenomenex Luna C18 75*30 mm*3 um; mobile phase: [water (FA)-ACN]; B %: 35%-65%, 8 min) to give the title compound (Compound 123) (8.9 mg, 20.85 umol, 2.98% yield, 100% purity) as a yellow solid. LCMS (ES⁺): m/z 427.1 [M+H]⁺.

### Example 123. Synthesis of (R)-1-(5-((5-chloro-4-(3-(pyridin-2-yl)piperidin-1-yl)pyrimidin-2-yl)amino)pyridin-3-yl)pyrrolidin-2-one (Compound 124)

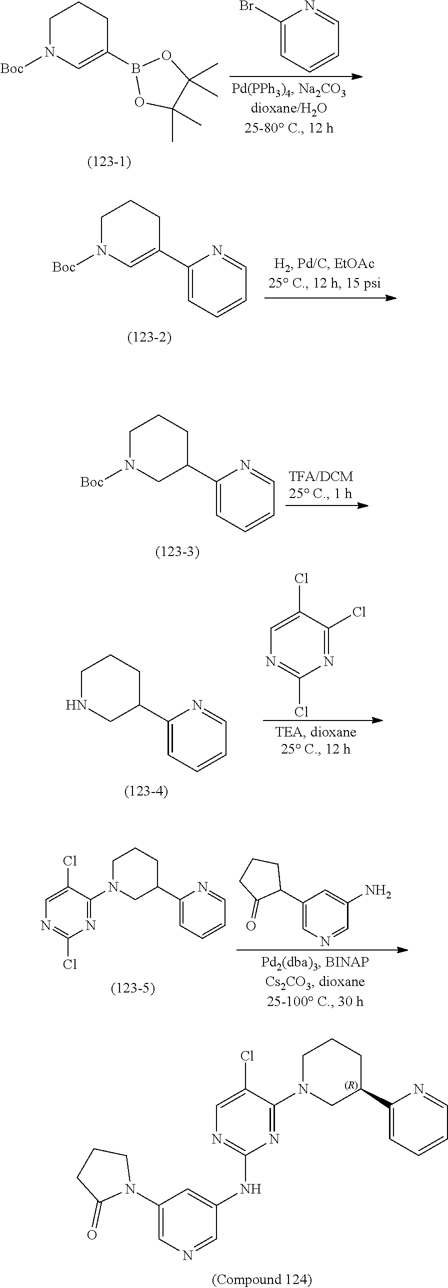

Synthesis of tert-butyl 5′,6′-dihydro-[2,3′-bipyridine]-1′(4′H)-carboxylate (123-2). The reaction were set up to two batches. To a solution of tert-butyl 5-(4,4,5,5-tetramethyl-1,3,2-dioxaborolan-2-yl)-3,4-dihydropyridine-1(2H)-carboxylate (123-1) (500 mg, 1.62 mmol, 1.00 eq) in H₂O (4 mL) and dioxane (12 mL) were added Pd(PPh₃)₄ (18.7 mg, 16.2 umol, 0.0100 eq), Na₂CO₃ (343 mg, 3.23 mmol, 2.00 eq), 2-bromopyridine (255 mg, 1.62 mmol, 154 μL, 1.00 eq) at 25° C. The mixture was stirred at 80° C. for 12 h under N₂ at atmosphere. LCMS showed tert-butyl 5-(4,4,5,5-tetramethyl-1,3,2-dioxaborolan-2-yl)-3,4-dihydropyridine-1(2H)-carboxylate (123-1) was consumed, desired target MS was detected. Two batches in parallel were combined. The mixture was concentrated in vacuo, H₂O (25 mL) was added to the solution, the mixture was extracted with EtOAc (25 mL*3), the combined organic layers were dried over Na₂SO₄, then concentrated in vacuo. The mixture was purified by prep-TLC (SiO₂, PE/EtOAc=10/1) to give the title compound (123-2) (360 mg, crude) as a yellow oil. LCMS (ES⁺): m/z 261.1 [M+H]⁺.

Synthesis of tert-butyl 3-(pyridin-2-yl)piperidine-1-carboxylate (123-3). To a solution of tert-butyl 5′,6′-dihydro-[2,3′-bipyridine]-1′(4′H)-carboxylate (123-2) (360 mg, 1.38 mmol, 1.00 eq) in EtOAc (10 mL) was added 10% Pd/C (360 mg, 50% purity) at 25° C., then the mixture was stirred at 25° C. for 12 h under H₂ at 15 Psi. LCMS showed tert-butyl 5′,6′-dihydro-[2,3′-bipyridine]-1′(4′H)-carboxylate (123-2) was consumed, desired target MS was detected. The mixture was filtered, the filtrate was concentrated in vacuo to give the title compound (123-3) (320 mg, crude) as a yellow oil. LCMS (ES⁺): m/z 263.1 [M+H]⁺.

Synthesis of 2-(piperidin-3-yl)pyridine (123-4). A solution of tert-butyl 3-(pyridin-2-yl)piperidine-1-carboxylate (123-3) (320 mg, 1.22 mmol, 1.00 eq) in DCM (5 mL) and TFA (1 mL) was stirred at 25° C. for 1 h. LCMS showed tert-butyl 3-(pyridin-2-yl)piperidine-1-carboxylate (123-3) was consumed, desired target MS was detected. The mixture was concentrated in vacuo to give the title compound (123-4) (337 mg, crude, TFA) as a yellow oil. LCMS (ES⁺): m/z 163.1 [M+H]⁺.

Synthesis of 2,5-dichloro-4-(3-(pyridin-2-yl)piperidin-1-yl)pyrimidine (123-5). To a solution of 2-(piperidin-3-yl)pyridine (123-4) (337 mg, 1.22 mmol, 1.00 eq, TFA) and 2,4,5-trichloropyrimidine (201 mg, 1.10 mmol, 0.900 eq) in dioxane (5 mL) was added TEA (247 mg, 2.44 mmol, 340 μL, 2.00 eq) at 25° C., the mixture was stirred at 25° C. for 12 h. LCMS showed 2-(piperidin-3-yl)pyridine (123-4) was consumed, desired target MS was detected. H₂O (15 mL) was added to the solution, the mixture was extracted with EtOAc (15 mL*3), the combined organic layers were dried over Na₂SO₄, then concentrated in vacuo. The mixture was purified by prep-TLC (SiO₂, PE/EtOAc=5/1) to give the title compound (123-5) (216 mg, crude) as a yellow oil. LCMS (ES⁺): m/z 309.0 [M+H]⁺.

Synthesis of (R)-1-(5-((5-chloro-4-(3-(pyridin-2-yl)piperidin-1-yl)pyrimidin-2-yl)amino)pyridin-3-yl)pyrrolidin-2-one (Compound 124). To a solution of 2,5-dichloro-4-(3-(pyridin-2-yl)piperidin-1-yl)pyrimidine (123-5) (190 mg, 614.50 umol, 1.00 eq) and 1-(5-amino-3-pyridyl)pyrrolidin-2-one (156 mg, 615 umol, 70% purity, 1.00 eq) in dioxane (10 mL) were added BINAP (23.0 mg, 36.9 umol, 0.0600 eq), Cs₂CO₃ (400 mg, 1.23 mmol, 2.00 eq), Pd₂(dba)₃ (22.5 mg, 24.6 umol, 0.0400 eq) at 25° C., the mixture was stirred at 100° C. for 12 h under N₂ atmosphere. LCMS showed 2,5-dichloro-4-(3-(pyridin-2-yl)piperidin-1-yl)pyrimidine (123-5) was remained, desired target MS was not detected. Then 1-(5-amino-3-pyridyl)pyrrolidin-2-one (109 mg, 615 umol, 1.00 eq), BINAP (23.0 mg, 36.9 umol, 0.0600 eq), Cs₂CO₃ (400 mg, 1.23 mmol, 2.00 eq), Pd₂(dba)₃ (22.5 mg, 24.6 umol, 0.0400 eq) was added to the mixture, the mixture was stirred 100° C. for 12 h under N₂ atmosphere. LCMS showed 2,5-dichloro-4-(3-(pyridin-2-yl)piperidin-1-yl)pyrimidine (123-5) was remained, desired target MS was detected, then 1-(5-amino-3-pyridyl)pyrrolidin-2-one (54.5 mg, 307 umol, 0.500 eq), BINAP (11.5 mg, 18.4 umol, 0.0300 eq), Cs₂CO₃ (200 mg, 614 umol, 1.00 eq), Pd₂(dba)₃ (11.3 mg, 12.3 umol, 0.0200 eq) was added to the mixture, the mixture was stirred 100° C. for 6 h under N₂ atmosphere. LCMS showed 2,5-dichloro-4-(3-(pyridin-2-yl)piperidin-1-yl)pyrimidine (123-5) was remained, desired target MS was detected. H₂O (10 mL) was added to the solution, the mixture was extracted with EtOAc (10 mL*3), the combined organic lays were dried over Na₂SO₄, then concentrated in vacuo to give the crude product. The mixture was purified by MPLC (SiO₂, Petroleum ether/Ethyl acetate=1/0 to 1/1) to give 120 mg isomer as a yellow solid. Then the isomer was separated by chiral SFC (Instrument: CAS-TJ-ANA-SFC-N (Waters UPCC with PDA); column: DAICEL CHIRALPAK IF (250 mm*30 mm, 10 um); mobile phase: [Heptane-(EtOH:ACN=4:1)]; B %: 50%-50%, 10 min(separate method from). Two enantiomers were assigned arbitrarily. The title compound (Compound 124) (31.9 mg, 70.5 umol, 11.5% yield, 99.5% purity, 100% ee %) (RT=3.946) was obtained as a white solid. LCMS (ES⁺): m/z 450.0 [M+H]⁺.

### Example 124. Synthesis of (S)-1-(5-((5-chloro-4-(3-(pyridin-2-yl)piperidin-1-yl)pyrimidin-2-yl)amino)pyridin-3-yl)pyrrolidin-2-one (Compound 125)

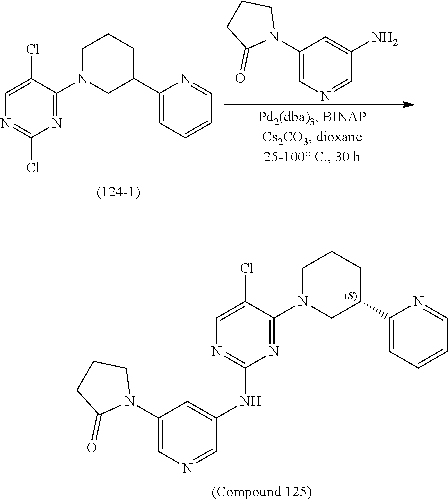

Synthesis of (S)-1-(5-((5-chloro-4-(3-(pyridin-2-yl)piperidin-1-yl)pyrimidin-2-yl)amino)pyridin-3-yl)pyrrolidin-2-one (Compound 125). To a solution of 2,5-dichloro-4-(3-(pyridin-2-yl)piperidin-1-yl)pyrimidine (124-1) (190 mg, 615 umol, 1.00 eq) and 1-(5-aminopyridin-3-yl)pyrrolidin-2-one (156 mg, 615 umol, 70.0% purity, 1.00 eq) in dioxane (10 mL) were added BINAP (23.0 mg, 36.9 umol, 0.0600 eq), Cs₂CO₃ (400 mg, 1.23 mmol, 2.00 eq), Pd₂(dba)₃ (22.5 mg, 24.6 umol, 0.0400 eq) at 25° C., the mixture was stirred at 100° C. for 12 h under N₂ atmosphere. LCMS showed 2,5-dichloro-4-(3-(pyridin-2-yl)piperidin-1-yl)pyrimidine (124-1) remained, desired target MS was not detected. Then 1-(5-aminopyridin-3-yl)pyrrolidin-2-one (156 mg, 615 umol, 70% purity, 1.00 eq), BINAP (23.0 mg, 36.9 umol, 0.0600 eq), Cs₂CO₃ (400 mg, 1.23 mmol, 2.00 eq), Pd₂(dba)₃ (22.5 mg, 24.6 umol, 0.0400 eq) was added to the mixture, the mixture was stirred 100° C. for 12 h under N₂ atmosphere. LCMS showed 2,5-dichloro-4-(3-(pyridin-2-yl)piperidin-1-yl)pyrimidine (124-1) was remained, desired target MS was detected, then 1-(5-aminopyridin-3-yl)pyrrolidin-2-one (54.5 mg, 307 umol, 0.500 eq), BINAP (11.5 mg, 18.4 umol, 0.0300 eq), Cs₂CO₃ (200 mg, 614 umol, 1.00 eq), Pd₂(dba)₃ (11.3 mg, 12.3 umol, 0.0200 eq) was added to the mixture, the mixture was stirred 100° C. for 6 h under N₂ atmosphere. LCMS showed 2,5-dichloro-4-(3-(pyridin-2-yl)piperidin-1-yl)pyrimidine (124-1) was remained, desired target MS was detected. H₂O (10 mL) was added to the solution, the mixture was extracted with EtOAc (10 mL*3), the combined organic lays were dried over Na₂SO₄, then concentrated in vacuo. The mixture was purified by MPLC (SiO₂, Petroleum ether/Ethyl acetate=1/0 to 1/1) to give 120 mg isomer as a yellow solid. Then the isomer was separated by chiral SFC (Instrument: CAS-TJ-ANA-SFC-N (Waters UPCC with PDA); column: DAICEL CHIRALPAK IF (250 mm*30 mm, 10 um); mobile phase: [Heptane-(EtOH:ACN=4:1)]; B %: 50%-50%, 10 min(separate method from). Two enantiomers were assigned arbitrarily. The title compound (Compound 125) (10.3 mg, 22.8 umol, 3.71% yield, 99.5% purity, 93.66% ee %) (RT=4.480) was obtained as a yellow solid. LCMS (ES⁺): m/z 450.0 [M+H]⁺.

### Example 125. of 1-(5-((5-chloro-4-(3-cyclopropylpiperidin-1-yl)pyrimidin-2-yl)amino)pyridin-3-yl)pyrrolidin-2-one (Compound 126)

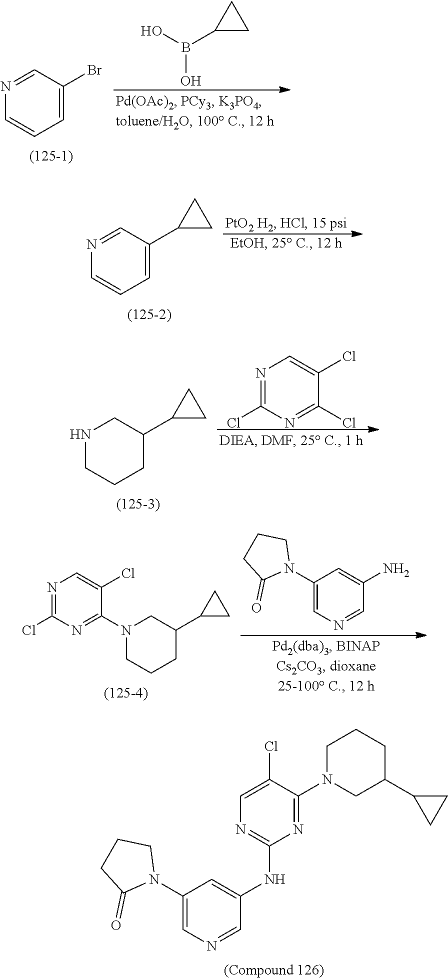

Synthesis of 3-cyclopropylpyridine (125-2). To a solution of 3-bromopyridine (125-1) (5 g, 31.6 mmol, 3.05 mL, 1.00 eq) and cyclopropylboronic acid (2.99 g, 34.8 mmol, 1.10 eq) in Tol. (50 mL) and H₂O (10 mL) were added Pd(OAc)₂ (710 mg, 3.16 mmol, 0.100 eq), K₃PO₄ (13.4 g, 63.2 mmol, 2.00 eq) and PCy₃ (887 mg, 3.16 mmol, 1.03 mL, 0.100 eq) at 25° C. The mixture was stirred at 100° C. for 12 h. LCMS showed the desired mass was detected. The reaction mixture was diluted with H₂O 50 mL and extracted with EtOAc (40 mL*3). The combined organic layers were dried over Na₂SO₄, filtered and concentrated under reduced pressure. The residue was purified by MPLC (SiO₂, PE/EtOAc=1/1) to give the title compound (125-2) (3.20 g, 14.5 mmol, 22.9% yield, 54.0% purity) as a yellow oil. LCMS (ES⁺): m/z 120.4 [M+H]⁺.

Synthesis of 3-cyclopropylpiperidine (125-3). To a solution of 3-cyclopropylpyridine (125-2) (1.00 g, 8.39 mmol, 1.00 eq) in EtOH (20.0 mL) was added HCl (12.0 M, 1.05 mL, 1.50 eq) at 25° C. The mixture was added PtO₂ (190 mg, 839 umol, 0.100 eq) under H₂ (15 Psi), then the mixture stirred at 25° C. for 12 h. LCMS showed the desired mass was detected. The reaction mixture was filtered and the filtrate was concentrated under reduced pressure to give the title compound (125-3)(1.4 g, crude) as a black oil. LCMS (ES⁺): m/z 126.4 [M+H]⁺.

Synthesis of 2,5-dichloro-4-(3-cyclopropylpiperidin-1-yl)pyrimidine (125-4) To a solution of 3-cyclopropylpiperidine (125-3) (650 mg, 5.19 mmol, 1.00 eq) and 2,4,5-trichloropyrimidine (1.05 g, 5.71 mmol, 1.10 eq) in DMF (10 mL) was added DIEA (670 mg, 5.19 mmol, 904 μL, 1.00 eq) at 25° C. The mixture was stirred at 25° C. for 1 h. LCMS showed the desired mass was detected. The reaction mixture was diluted with H₂O 10 mL and extracted with EtOAc (10 mL*3). The combined organic layers were dried over Na₂SO₄, filtered and concentrated under reduced pressure. The residue was purified by column chromatography (SiO₂, Petroleum ether/Ethyl acetate=20/1 to 5/1) to give the title compound (125-4) (1.30 g, 1.56 mmol, 15.0% yield, 32.6% purity) was obtained as a colourless oil. LCMS (ES⁺): m/z 272.2 [M+H]⁺.

Synthesis of 1-[5-[[5-chloro-4-(3-cyclopropyl-1-piperidyl)pyrimidin-2-yl]amino]-3-pyridyl]pyrrolidin-2-one (Compound 126). To a solution of 2,5-dichloro-4-(3-cyclopropylpiperidin-1-yl)pyrimidine (125-4) (150 mg, 551 umol, 1.00 eq) and 1-(5-amino-3-pyridyl)pyrrolidin-2-one (146 mg, 826 umol, 1.50 eq) in dioxane (3 mL) was added BINAP (34.3 mg, 55.1 umol, 0.100 eq) and Pd₂(dba)₃ (50.4 mg, 55.1 umol, 0.100 eq) and Cs₂CO₃ (538 mg, 1.65 mmol, 3.00 eq) under N₂ at 25° C., then The mixture was stirred for 12 h at 100° C. LCMS showed the desired mass was detected. The reaction mixture was filtered and concentrated under reduced pressure. The residue was purified by prep-HPLC (column: Phenomenex Luna C18 75*30 mm*3 um; mobile phase: [water (FA)-ACN]; B %: 35%-65%, 8 min) to give the title compound (Compound 126) (21.3 mg, 51.5 umol, 9.36% yield, 100% purity) as a white solid. LCMS (ES⁺): m/z 413.1 [M+H]⁺.

### Example 126. Synthesis of 1-(5-((5-chloro-4-(3-cyclopentylpiperidin-1-yl)pyrimidin-2-yl)amino)pyridin-3-yl)pyrrolidin-2-one (Compound 131)

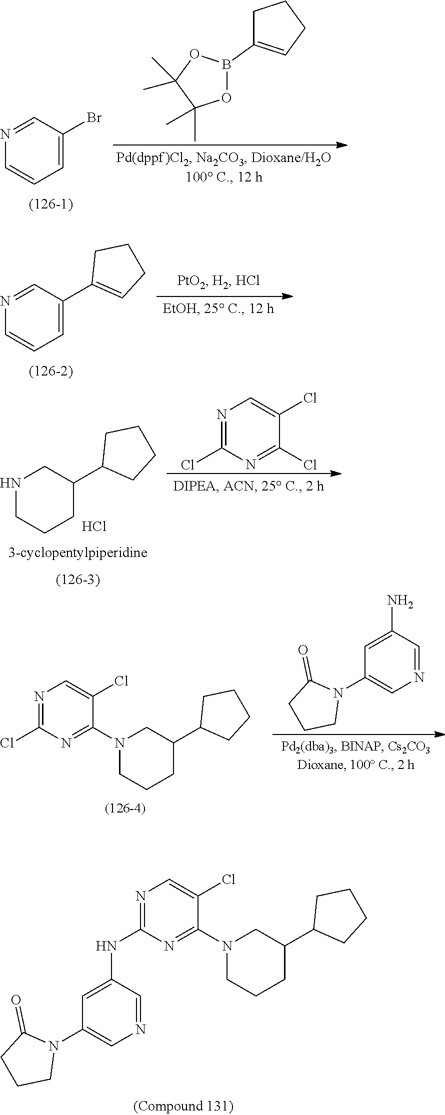

Synthesis of 3-(cyclopent-1-en-1-yl)pyridine (126-2). To a solution of 3-bromopyridine (126-1) (5.00 g, 31.6 mmol, 3.05 mL, 1.00 eq) and 2-(cyclopenten-1-yl)-4,4,5,5-tetramethyl-1,3,2-dioxaborolane (7.37 g, 37.9 mmol, 1.20 eq) in dioxane (100 mL) and H₂O (20.0 mL) was added Pd(dppf)Cl₂ (2.32 g, 3.16 mmol, 0.100 eq) and Na₂CO₃ (10.1 g, 94.9 mmol, 3.00 eq) at 25° C. The mixture was stirred at 100° C. for 12 h under N₂. LC-MS showed 3-bromopyridine (126-1) was consumed completely and the desired mass was detected. The reaction mixture was diluted with water 100 mL and extracted with EtOAc mL (100 mL*3). The combined organic layers were washed with brine (100 mL*3), dried over Na₂SO₄, filtered and concentrated under reduced pressure. The residue was purified by column chromatography (SiO₂, Petroleum ether/Ethyl acetate=1:1 to 1:2) to give the title compound (126-2) (6.5 g, crude) as a yellow oil.

Synthesis of 3-cyclopentylpiperidine (126-3). To a solution of PtO₂ (300 mg, 1.32 mmol, 1.28 eq) and HCl (12.0 M, 1.29 mL, 1.50 eq) in EtOH (10.0 mL) was added 3-(cyclopent-1-en-1-yl)pyridine (126-2) (1.50 g, 10.3 mmol, 1.00 eq) under N₂ atmosphere. The suspension was degassed and purged with H₂ for 5 times. The mixture was stirred under H₂ 15 Psi at 25° C. for 12 h. LC-MS showed 3-(cyclopent-1-en-1-yl)pyridine (126-2) was consumed completely and the desired mass was detected. The reaction mixture was filtered and concentrated under reduced pressure to give the title compound (126-3) (2.50 g, crude, HCl) as a white solid. LCMS (ES⁺): m/z 154.4 [M+H]⁺.

Synthesis of 2,5-dichloro-4-(3-cyclopentylpiperidin-1-yl)pyrimidine (126-4). To a solution of 3-cyclopentylpiperidine (126-4) (2.00 g, 13.0 mmol, 1.00 eq) in ACN (5.00 mL) was added DIEA (8.43 g, 65.2 mmol, 11.3 mL, 5.00 eq) and 2,4,5-trichloropyrimidine (2.39 g, 13.1 mmol, 1.00 eq) at 25° C. The mixture was stirred at 25° C. for 2 h. LC-MS showed 3-cyclopentylpiperidine (126-3) was consumed completely and the desired mass was detected. Water (50.0 mL) was added to the solution, the mixture was extracted with ethyl acetate (50.0 mL*3), the combined organic layers were dried over Na₂SO₄, and concentrated under reduced pressure. The residue was purified by column chromatography (SiO₂, Petroleum ether:Ethyl acetate=10:1 to 8:1) to give the title compound (126-4) (1.50 g, 4.45 mmol, 34.1% yield, 89.0% purity) as a white solid. LCMS (ES⁺): m/z 300.0 [M+H]⁺.

Synthesis of 1-(5-((5-chloro-4-(3-cyclopentylpiperidin-1-yl)pyrimidin-2-yl)amino)pyridin-3-yl)pyrrolidin-2-one (Compound 131). To a solution of 2,5-dichloro-4-(3-cyclopentylpiperidin-1-yl)pyrimidine (126-4) (200 mg, 666 umol, 1.00 eq) in dioxane (3.00 mL) was added Pd₂(dba)₃ (122 mg, 133 umol, 0.200 eq), 1-(5-amino-3-pyridyl)pyrrolidin-2-one (118 mg, 666 umol, 1.00 eq), Cs₂CO₃ (651 mg, 2.00 mmol, 3.00 eq) and BINAP (82.9 mg, 133 umol, 0.200 eq) in N₂ at 25° C. The mixture was stirred at 100° C. for 2 h. LC-MS showed 2,5-dichloro-4-(3-cyclopentylpiperidin-1-yl)pyrimidine (126-4) was consumed completely and the desired mass was detected. Water (50.0 mL) was added to the solution, the mixture was extracted with ethyl acetate (50.0 mL*3), the combined organic layers were dried over Na₂SO₄, and concentrated under reduced pressure. The residue was purified by prep-HPLC (column: Phenomenex Luna 80*30 mm*3 um; mobile phase: [water (TFA)-ACN]; B %: 50%-80%, 8 min) to give the title compound (Compound 131) (50.3 mg, 107 umol, 16.1% yield, 94.0% purity) as light yellow solid. LCMS (ES⁺): m/z 441.2 [M+H]⁺.

### Example 127. Synthesis of 2-[5-[[5-chloro-4-(3-phenylphenyl)pyrimidin-2-yl]amino]-3-pyridyl]-8-methyl-2,8-diazaspiro[4.5]decan-1-one (Compound 132)

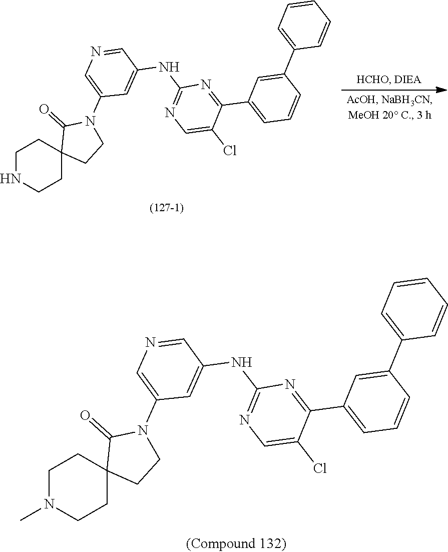 [SMILES: O=C1N(c2cncc(Nc3ncc(Cl)c(-c4cccc(-c5ccccc5)c4)n3)c2)CCC12CCNCC2 ; CN1CCC2(CC1)CCN(c1cncc(Nc3ncc(Cl)c(-c4cccc(-c5ccccc5)c4)n3)c1)C2=O]

Synthesis of 2-[5-[[5-chloro-4-(3-phenylphenyl)pyrimidin-2-yl]amino]-3-pyridyl]-8-methyl-2,8-diazaspiro[4.5]decan-1-one (Compound 132). To a solution of 2-(5-((4-([1,1′-biphenyl]-3-yl)-5-chloropyrimidin-2-yl)amino)pyridin-3-yl)-8-methyl-2,8-diazaspiro[4.5]decan-1-one (127-1) (200 mg, 320 umol, 1.00 eq, TFA) in MeOH (1 mL) were added DIEA (82.6 mg, 639 umol, 111 μL, 2.00 eq), AcOH (19.2 mg, 320 umol, 18.3 uL, 1.00 eq) and HCHO (9.65 mg, 321 umol, 8.85 uL, 1.00 eq) at 20° C. The mixture was stirred at 20° C. for 2 h. NaBH₃CN (60.3 mg, 960 umol, 3.00 eq) was added to the mixture at 20° C. The mixture was stirred at 20° C. for 1 h. LCMS showed the desired compound was detected. The mixture was concentrated under reduced pressure. The residue was purified by prep-HPLC (column: Phenomenex Luna C18 75*30 mm*3 um; mobile phase: [water (FA)-ACN]; B %: 20%-55%, 8 min) to give the title compound (Compound 132) (38.0 mg, 71.7 umol, 22.4% yield, 99.0% purity) as a white solid. LCMS (ES⁺): m/z 525.1 [M+H]⁺.

### Example 128. Synthesis of 8-acetyl-2-[5-[[5-chloro-4-(3-phenylphenyl)pyrimidin-2-yl]amino]-3-pyridyl]-2,8-diazaspiro[4.5]decan-1-one (Compound 133)

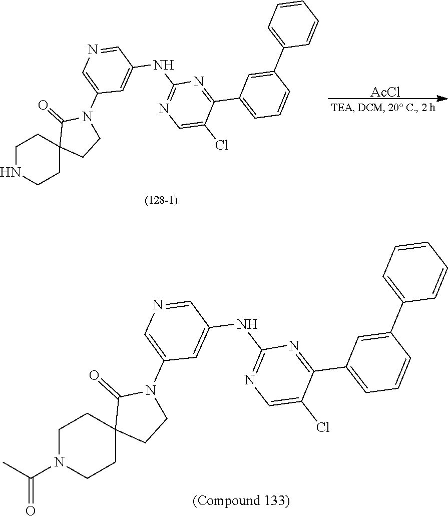 [SMILES: "AcCl" ; O=C1N(c2cncc(Nc3ncc(Cl)c(-c4cccc(-c5ccccc5)c4)n3)c2)CCC12CCNCC2 ; CC(=O)N1CCC2(CC1)CCN(c1cncc(Nc3ncc(Cl)c(-c4cccc(-c5ccccc5)c4)n3)c1)C2=O]

Synthesis of 8-acetyl-2-[5-[[5-chloro-4-(3-phenylphenyl)pyrimidin-2-yl]amino]-3-pyridyl]-2,8-diazaspiro[4.5]decan-1-one (Compound 133). To a solution of 2-[5-[[5-chloro-4-(3-phenylphenyl)pyrimidin-2-yl]amino]-3-pyridyl]-2,8-diazaspiro[4.5]decan-1-one (128-1) (200 mg, 391 umol, 1.00 eq) and acetyl chloride (9.22 mg, 117 umol, 8.38 uL, 0.300 eq) in DCM (1 mL) was added TEA (119 mg, 1.18 mmol, 164 μL, 3.00 eq) at 20° C. The mixture was stirred at 20° C. for 2 h. LCMS showed that the desired compound was detected. The reaction mixture was concentrated under reduced pressure. The residue was purified by prep-HPLC (column: Waters Xbridge BEH C18 100*30 mm*10 um; mobile phase: [water (NH₄HCO₃)-ACN]; B %: 50%-70%, 8 min) to give the title compound (Compound 133) (24.1 mg, 43.2 umol, 11.1% yield, 99.2% purity) as a white solid. LCMS (ES⁺): m/z 553.0 [M+H]⁺.

### Example 129. Synthesis of 1-[5-[[5-chloro-4-[2-(2-pyridyl)morpholin-4-yl]pyrimidin-2-yl]amino]-3-pyridyl]pyrrolidin-2-one (Compound 134)

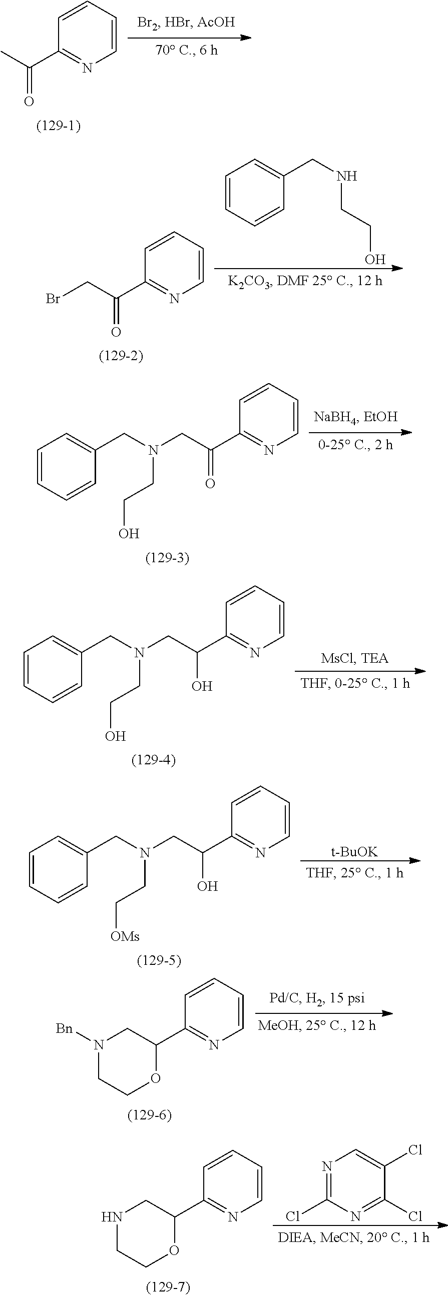

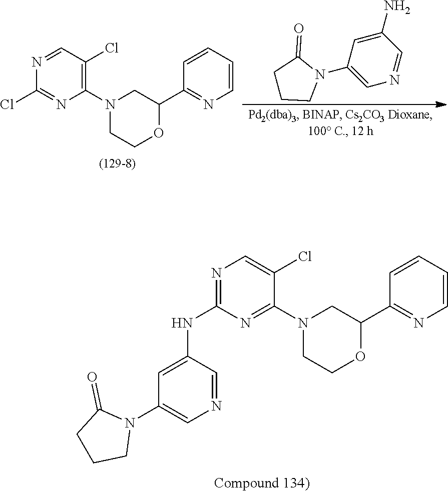

Synthesis of 2-bromo-1-(2-pyridyl)ethanone (129-2). These were two parallel reactions: To a mixture of 1-(2-pyridyl)ethanone (129-1) (10.0 g, 82.5 mmol, 9.26 mL, 1.00 eq) in AcOH (100 mL) was added HBr (16.7 g, 82.5 mmol, 11.2 mL, 40% purity, 1.00 eq) and Br₂ (19.7 g, 123 mmol, 6.38 mL, 1.50 eq) at 25° C. The mixture was stirred at 70° C. for 6 h. LCMS showed the desired mass was detected. The reaction mixture was quenched by addition H₂O 200 mL, and then diluted with H₂O 200 mL and extracted with DCM (200 mL*3). The combined organic layers were dried over Na₂SO₄, filtered and concentrated under reduced pressure. The residue was purified by column chromatography (SiO₂, Petroleum ether/Ethyl acetate=20/1 to 1/1) to give the title compound (129-2) (32.0 g, 159 mmol, 96.8% yield) as a colorless oil. LCMS (ES⁺): m/z 200.2 [M+H]⁺.

Synthesis of 2-[benzyl(2-hydroxyethyl)amino]-1-(2-pyridyl)ethanone (129-3). These were four parallel reactions. To a solution of 2-bromo-1-(2-pyridyl)ethanone (129-2) (7.00 g, 34.9 mmol, 1.00 eq) in DMF (80 mL) was added K₂CO₃ (8.13 g, 58.8 mmol, 1.68 eq) and 2-(benzylamino)ethanol (4.25 g, 28.1 mmol, 3.97 mL, 8.03e-1 eq) at 25° C. The mixture was stirred at 25° C. for 12 h. LCMS showed the desired mass was detected. H₂O 200 mL was added to the mixture, and extracted with DCM (200 mL*3). The combined organic layers were dried over Na₂SO₄, filtered and concentrated under reduced pressure. The residue was purified by column chromatography (SiO₂, Petroleum ether/Ethyl acetate=1/1) to give the title compound (129-3) (20.0 g, 73.9 mmol, 52.8% yield) as a yellow oil. LCMS (ES⁺): m/z 271.3 [M+H]⁺.

Synthesis of 2-[benzyl(2-hydroxyethyl)amino]-1-(2-pyridyl)ethanol (129-4). To a solution of 2-[benzyl(2-hydroxyethyl)amino]-1-(2-pyridyl)ethanone (129-3) (4.00 g, 14.8 mmol, 1.00 eq) in EtOH (40 mL) was added NaBH₄ (1.12 g, 29.6 mmol, 2.00 eq) at 0° C. The mixture was stirred at 25° C. for 2 h. LCMS showed the desired mass was detected. The reaction mixture was quenched by addition 0.5 N HCl solution 20 mL, the mixture was neutralized with 0.5 N NaOH (20 mL) and extracted with EtOAc (40 mL*3). The combined organic layers were washed with sat. aq. NaCl (10 mL), dried over Na₂SO₄, filtered and concentrated under reduced pressure. The residue was purified by column chromatography (SiO₂, Petroleum ether/Ethyl acetate=0/1) to give the title compound (129-4) (1.60 g, 5.87 mmol, 39.7% yield) as a yellow oil. LCMS (ES⁺): m/z 273.3 [M+H]⁺.

Synthesis of 2-[benzyl-[2-hydroxy-2-(2-pyridyl)ethyl]amino]ethyl methanesulfonate (129-5). To a solution of 2-[benzyl(2-hydroxyethyl)amino]-1-(2-pyridyl)ethanol (129-4) (600 mg, 2.20 mmol, 1.00 eq) in THE (3 mL) was added TEA (445 mg, 4.41 mmol, 613 μL, 2.00 eq) and methanesulfonyl chloride (201 mg, 1.76 mmol, 136 μL, 0.800 eq) at 0° C. The mixture was stirred at 25° C. for 1 h. TLC (SiO₂, PE/EtOAc=1/1) indicated one new spot formed. The reaction mixture was quenched by addition H₂O 10 mL, and extracted with EtOAc (10 mL*3). The combined organic layers were dried over Na₂SO₄, filtered and concentrated under reduced pressure to give the title compound (129-5) (700 mg, crude) as a yellow oil. LCMS (ES⁺): m/z 351.4 [M+H]⁺.

Synthesis of 4-benzyl-2-(2-pyridyl) morpholine (129-6). To a solution of 2-[benzyl-[2-hydroxy-2-(2-pyridyl) ethyl]amino]ethyl methanesulfonate (129-5) (700 mg, 2.00 mmol, 1.00 eq) in THE (6 mL) was added t-BuOK (224 mg, 2.00 mmol, 1.00 eq) at 25° C. The mixture was stirred at 25° C. for 1 h. LCMS showed the desired mass was detected. H₂O 10 mL was added to the mixture, and extracted with DCM (10 mL*3). The combined organic layers were dried over Na₂SO₄, filtered and concentrated under reduced pressure. The residue was purified by column chromatography (SiO₂, Petroleum ether/Ethyl acetate=0/1) to give the title compound (129-6) (300 mg, 1.18 mmol, 59.1% yield) as a yellow oil. LCMS (ES⁺): m/z 255.1 [M+H]⁺.

Synthesis of 2-(2-pyridyl) morpholine (129-7). To a solution of 4-benzyl-2-(2-pyridyl) morpholine (129-6) (200 mg, 786 umol, 1.00 eq) in MeOH (1 mL) was added 10% Pd/C (200 mg, 50% purity, 1.00 eq) was added at 25° C. The mixture degassed and purged with H₂ (15 psi) for 3 times, and then the mixture was stirred at 25° C. for 12 h under H₂ atmosphere. LCMS showed the desired mass was detected. The reaction mixture was filtered and the filtrate was concentrated under high vacuum to give the title compound (129-7) (120 mg, crude) as a yellow oil. LCMS (ES⁺): m/z 165.3 [M+H]⁺.

Synthesis of 4-(2,5-dichloropyrimidin-4-yl)-2-phenyl-morpholine (129-8). To a solution of 2-(2-pyridyl)morpholine (129-7) (120 mg, 730 umol, 1.00 eq) and 2,4,5-trichloropyrimidine (134 mg, 730 umol, 1.00 eq) in ACN (2 mL) was added DIEA (188.90 mg, 1.46 mmol, 254 μL, 2.00 eq) at 20° C. The mixture was stirred at 20° C. for 1 h. LCMS showed the desired mass was detected. H₂O 2 mL was added to the mixture, and extracted with DCM (10 mL*3). The combined organic layers were dried over Na₂SO₄, filtered and concentrated under reduced pressure. The residue was purified by prep-TLC (SiO₂, Petroleum ether/Ethyl acetate=2/1, R_{f}=0.36) to give the title compound (129-8) (100 mg, 322 umol, 44.1% yield) as a white solid. LCMS (ES⁺): m/z 311.2 [M+H]⁺.

Synthesis of 1-[5-[[5-chloro-4-[2-(2-pyridyl)morpholin-4-yl]pyrimidin-2-yl]amino]-3-pyridyl]pyrrolidin-2-one (Compound 134). To a solution of 4-(2,5-dichloropyrimidin-4-yl)-2-phenyl-morpholine (129-8) (50.0 mg, 161 umol, 1.00 eq), 1-(5-amino-3-pyridyl)pyrrolidin-2-one (39.9 mg, 225 umol, 1.40 eq), Cs₂CO₃ (131 mg, 402 umol, 2.50 eq), BINAP (10.0 mg, 16.1 umol, 0.100 eq) in dioxane (1 mL) and then the Pd₂(dba)₃ (14.7 mg, 16.1 umol, 0.100 eq) was added at 25° C. under N₂. The mixture was stirred at 100° C. for 12 h. LCMS showed the desired mass was detected. H₂O 10 mL was added to the mixture, and extracted with EtOAc (10 mL*3). The combined organic layers were dried over Na₂SO₄, filtered and concentrated under reduced pressure. The residue was purified by prep-HPLC (column: Phenomenex luna C18 80*40 mm*3 um; mobile phase: [water (FA)-ACN]; B %: 1%-40%, 8 min) to give the title compound (Compound 134) (7.7 mg, 16.4 umol, 10.1% yield, 96.4% purity) as a white solid. LCMS (ES⁺): m/z 452.1 [M+H]⁺.

### Example 130. Synthesis of 1-(5-((5-chloro-4-(1-cyclopentylpiperidin-3-yl)pyrimidin-2-yl)amino)pyridin-3-yl)pyrrolidin-2-one (Compound 135)

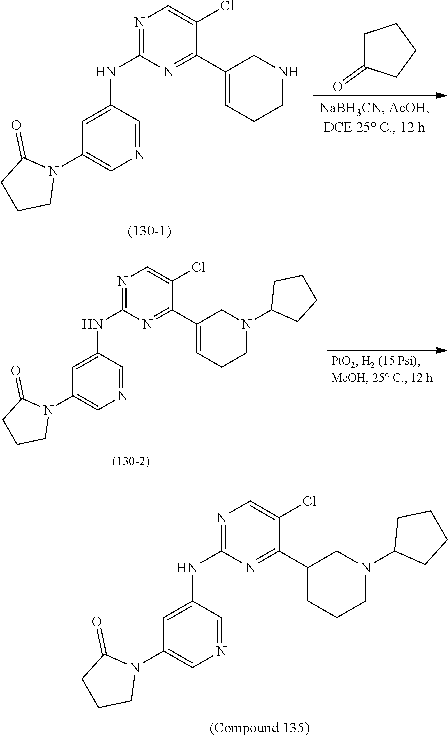 [SMILES: O=C1CCCN1c1cncc(Nc2ncc(Cl)c(C3=CCCNC3)n2)c1 ; O=C1CCCC1 ; O=C1CCCN1c1cncc(Nc2ncc(Cl)c(C3=CCCN(C4CCCC4)C3)n2)c1 ; O=C1CCCN1c1cncc(Nc2ncc(Cl)c(C3CCCN(C4CCCC4)C3)n2)c1]

Synthesis of 1-(5-((5-chloro-4-(1-cyclopentyl-1,2,5,6-tetrahydropyridin-3-yl)pyrimidin-2-yl)amino)pyridin-3-yl)pyrrolidin-2-one (130-2). To a solution of 1-(5-((5-chloro-4-(1,2,5,6-tetrahydropyridin-3-yl)pyrimidin-2-yl)amino)pyridin-3-yl)pyrrolidin-2-one (130-1) (500 mg, 1.35 mmol, 1.00 eq) in DCE (5 mL) was added cyclopentanone (226 mg, 2.70 mmol, 238 μL, 2.00 eq) and AcOH (809 mg, 13.4 mmol, 771 μL, 10.0 eq) at 25° C. the mixture was stirred at 25° C. for 0.5 h, then NaBH₃CN (169 mg, 2.70 mmol, 2.00 eq) was added to the mixture, The mixture was stirred at 25° C. for 11.5 h. LCMS showed the desired mass was detected. The reaction mixture was diluted with H₂O 10 mL and extracted with DCM (10 mL*3). The combined organic layers were dried over Na₂SO₄, filtered and concentrated under reduced pressure. The residue was purified by prep-TLC (SiO₂, Dichloromethane/Methanol=10/1, R_{f}=0.29) to give the title compound (130-2) (108 mg, crude) as a white solid. LCMS (ES⁺): m/z 439.2 [M+H]⁺.

Synthesis of 1-(5-((5-chloro-4-(1-cyclopentylpiperidin-3-yl)pyrimidin-2-yl)amino)pyridin-3-yl)pyrrolidin-2-one (Compound 135). To a solution of 1-(5-((5-chloro-4-(1-cyclopentyl-1,2,5,6-tetrahydropyridin-3-yl)pyrimidin-2-yl)amino)pyridin-3-yl)pyrrolidin-2-one (130-2) (108 mg, 246 umol, 1.00 eq) in MeOH (7 mL) was added PtO₂ (100 mg, 440 umol, 1.79 eq) at 25° C. under N₂ atmosphere. The suspension was degassed and purged with H₂ for 3 times. The mixture was stirred under H₂ (15 Psi) at 25° C. for 12 h. LCMS showed the desired mass was detected. The reaction mixture was filtered and the filtrate concentrated under high vacuum. The residue was purified by prep-HPLC (column: Phenomenex Luna C18 75*30 mm*3 um; mobile phase: [water (FA)-ACN]; B %: 1%-25%, 8 min) to give the title compound (Compound 135) (7.40 mg, 15.5 umol, 6.33% yield, 100% purity, 0.74 FA) as a white solid. LCMS (ES⁺): m/z 441.3 [M+H]⁺.

### Example 131. 1-(5-((5-chloro-4-(3-cyclopentylpyrrolidin-1-yl)pyrimidin-2-yl)amino)pyridin-3-yl)pyrrolidin-2-one (Compound 136)

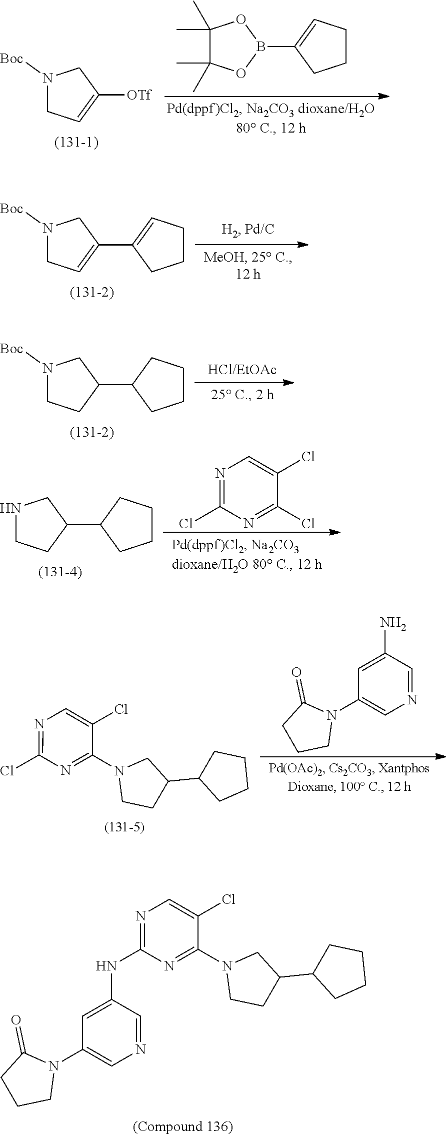

Synthesis of tert-butyl 3-(cyclopent-1-en-1-yl)-2,5-dihydro-1H-pyrrole-1-carboxylate (131-2). To a solution of tert-butyl 3-(trifluoromethylsulfonyloxy)-2,5-dihydropyrrole-1-carboxylate (131-1) (500 mg, 1.58 mmol, 1.00 eq) and 2-(cyclopenten-1-yl)-4,4,5,5-tetramethyl-1,3,2-dioxaborolane (306 mg, 1.58 mmol, 1.00 eq) in dioxane (5 mL) and H₂O (1.5 mL) were added Pd(dppf)Cl₂ (115 mg, 158 umol, 0.100 eq) and Na₂CO₃ (501 mg, 4.73 mmol, 3.00 eq) at 25° C. The mixture was stirred at 80° C. for 12 h under N₂ atmosphere. LCMS showed the desired mass was detected. The reaction mixture was partitioned between H₂O 10 mL and EtOAc 10 mL. The organic phase was separated, the aqueous was extracted with EtOAc (10 mL*3), the combined organic phase were dried over Na₂SO₄, filtered and the filtrate was concentrated under reduced pressure to give the title compound (131-2) (200 mg, 850 umol, 53.9% yield) as a white solid. LCMS (ES⁺): m/z 180.1 [M+H−56]⁺.

### Synthesis of tert-butyl 3-cyclopentylpyrrolidine-1-carboxylate (131-3)

To a solution of tert-butyl 3-(cyclopenten-1-yl)-2, 5-dihydropyrrole-1-carboxylate (131-2) (150 mg, 637 umol, 1.00 eq) in MeOH (3 mL) was added 10% Pd/C (150 mg, 50.0% purity) at 25° C. The mixture was stirred at 25° C. under H₂ (15 Psi) for 12 h. LCMS showed that tert-butyl 3-(cyclopenten-1-yl)-2, 5-dihydropyrrole-1-carboxylate (131-2) was consumed and the desired mass was detected. The reaction mixture was filtered and the filtrate was concentrated under reduced pressure to give the title compound (131-3) (150 mg, crude) as a white solid. LCMS (ES⁺): m/z 184.2 [M+H−56]⁺.

Synthesis of 3-cyclopentylpyrrolidine (131-4). A solution of tert-butyl 3-cyclopentylpyrrolidine-1-carboxylate (131-3) (150 mg, 626.69 umol, 1.00 eq) in HCl/EtOAc (10 mL, 4M) was stirred at 25° C. for 2 h. LCMS showed that tert-butyl 3-cyclopentylpyrrolidine-1-carboxylate was consumed and the desired mass was detected. The reaction mixture was concentrated under reduced pressure to give the title compound (131-4) (87.0 mg, crude) as a brown solid. LCMS (ES⁺): m/z 140.2 [M+H]⁺.

Synthesis of 2, 5-dichloro-4-(3-cyclopentylpyrrolidin-1-yl)pyrimidine (131-5). To a solution of 3-cyclopentylpyrrolidine (131-4) (87.0 mg, 625 umol, 1.00 eq) and 2, 4, 5-trichloropyrimidine (172 mg, 937 umol, 1.50 eq) in dioxane (10 mL) and H₂O (0.5 mL) were added Na₂CO₃ (199 mg, 1.87 mmol, 3.00 eq) and Pd(dppf)Cl₂ (45.7 mg, 62.5 umol, 0.100 eq) at 25° C. The mixture was stirred at 80° C. for 12 h under N₂ atmosphere. LCMS showed 3-cyclopentylpyrrolidine was consumed and the desired mass was detected. The reaction mixture was filtered and the filtrate was concentrated under reduced pressure to get a residue. The residue was purified by prep-TLC (SiO₂, Petroleum ether:Ethyl acetate=3:1) to give the title compound (131-5) (140 mg, 489 umol, 78.3% yield) as a white solid. LCMS (ES⁺): m/z 285.8 [M+H]⁺.

Synthesis of 1-(5-((5-chloro-4-(3-cyclopentylpyrrolidin-1-yl)pyrimidin-2-yl)amino)pyridin-3-yl)pyrrolidin-2-one (Compound 136). To a solution of 2,5-dichloro-4-(3-cyclopentylpyrrolidin-1-yl)pyrimidine (131-5) (110 mg, 384 umol, 1.00 eq) and 1-(5-amino-3-pyridyl)pyrrolidin-2-one (136 mg, 769 umol, 2.00 eq) in dioxane (6 mL) were added Pd(OAc)₂ (8.63 mg, 38.4 umol, 0.100 eq), Cs₂CO₃ (313 mg, 961 umol, 2.50 eq) and Xantphos (44.5 mg, 76.9 umol, 0.200 eq) at 25° C. The mixture was stirred at 100° C. for 12 h under N₂ atmosphere. LCMS showed 2,5-dichloro-4-(3-cyclopentylpyrrolidin-1-yl)pyrimidine was consumed and the desired mass was detected. The reaction mixture was filtered and the filtrate was concentrated under reduced pressure to remove solvent. The residue was purified by prep-HPLC (column: Phenomenex Luna 80*30 mm*3 um; mobile phase: [water (FA)-ACN]; B %: 30%-60%, 8 min) to give the title compound (Compound 136) (14.4 mg, 33.7 umol, 8.78% yield, 100% purity) as a white solid. LCMS (ES⁺): m/z 427.1 [M+H]⁺.

### Example 132 Synthesis of 1-(5-((5-chloro-4-(1-cyclobutyl-1H-pyrazol-4-yl)pyrimidin-2-yl)amino)pyridin-3-yl)pyrrolidin-2-one (Compound 137)

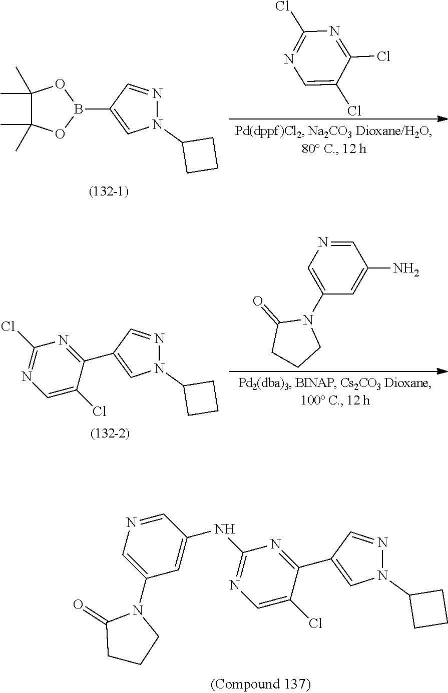 [SMILES: CC1(C)OB(c2cnn(C3CCC3)c2)OC1(C)C ; Clc1ncc(Cl)c(Cl)n1 ; Clc1ncc(Cl)c(-c2cnn(C3CCC3)c2)n1 ; Nc1cncc(N2CCCC2=O)c1 ; O=C1CCCN1c1cncc(Nc2ncc(Cl)c(-c3cnn(C4CCC4)c3)n2)c1]

Synthesis of 2,5-dichloro-4-(I-cyclobutyl-1H-pyrazol-4-yl)pyrimidine (132-2). To a solution of 1-cyclobutyl-4-(4,4,5,5-tetramethyl-1,3,2-dioxaborolan-2-yl)-1H-pyrazole (132-1) (400 mg, 1.61 mmol, 1.00 eq), 2,4,5-trichloropyrimidine (325 mg, 1.77 mmol, 1.10 eq), Pd(dppf)Cl₂ (117 mg, 161 umol, 0.100 eq) and Na₂CO₃ (512 mg, 4.84 mmol, 3.00 eq) in dioxane (5 mL) and H₂O (1 mL) was degassed and purged with N₂ at 25° C. for 3 times, and then the mixture was stirred at 80° C. for 12 h under N₂ atmosphere. LCMS showed 1-cyclobutyl-4-(4,4,5,5-tetramethyl-1,3,2-dioxaborolan-2-yl)-1H-pyrazole (132-1) was consumed completely and the desired mass was detected. H₂O 10 mL was added to the mixture, and then diluted with H₂O 10 mL and extracted with EtOAc (10 mL*3). The combined organic layers were washed with sat. aq. NaCl (10 mL), dried over Na₂SO₄, filtered and concentrated under reduced pressure. The residue was purified by prep-TLC (SiO₂, Petroleum ether/Ethyl acetate=3/1, Rf=0.31) to give the title compound (132-2) (350 mg, crude) as a yellow oil. LCMS (ES⁺): m/z 269.0 [M+H]⁺

Synthesis of 1-(5-((5-chloro-4-(1-cyclobutyl-1H-pyrazol-4-yl)pyrimidin-2-yl)amino)pyridin-3-yl)pyrrolidin-2-one (Compound 137). To a solution of 2,5-dichloro-4-(1-cyclobutyl-1H-pyrazol-4-yl)pyrimidine (132-2) (200 mg, 743 umol, 1.00 eq), 1-(5-aminopyridin-3-yl)pyrrolidin-2-one (131 mg, 743 umol, 1.00 eq), Pd₂(dba)₃ (68.1 mg, 74.3 umol, 0.100 eq), BINAP (46.3 mg, 74.3 umol, 0.100 eq) and Cs₂CO₃ (726 mg, 2.23 mmol, 3.00 eq) in dioxane (10 mL) was degassed and purged with N₂ at 25° C. for 3 times, and then the mixture was stirred at 100° C. for 12 h under N₂ atmosphere. LCMS showed 2,5-dichloro-4-(1-cyclobutyl-1H-pyrazol-4-yl)pyrimidine (132-2) was consumed completely and the desired mass was detected. H₂O 5 mL was added to the mixture, and then diluted with H₂O 5 mL and extracted with EtOAc (5 mL*3). The combined organic layers were washed with sat. aq. NaCl (10 mL), dried over Na₂SO₄, filtered and concentrated under reduced pressure. The residue was purified by prep-HPLC (Phenomenex Luna C18 200*40 mm*10 um; mobile phase: [water (FA)-ACN]; B %: 20%-50%, 8 min) to give the title compound (Compound 137) (14.4 mg, 33.4 umol, 4.50% yield, 95.2% purity) as a white solid. LCMS (ES⁺): m/z 410.1 [M+H]⁺

### Example 133. Synthesis of 1-[5-[[5-chloro-4-(1-cyclopentylpyrazol-4-yl)pyrimidin-2-yl]amino]-3-pyridyl]pyrrolidin-2-one (Compound 138)

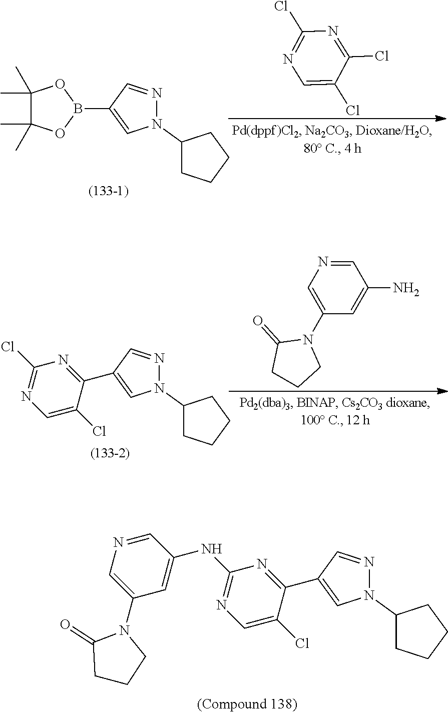 [SMILES: CC1(C)OB(c2cnn(C3CCCC3)c2)OC1(C)C ; Clc1ncc(Cl)c(Cl)n1 ; Clc1ncc(Cl)c(-c2cnn(C3CCCC3)c2)n1 ; Nc1cncc(N2CCCC2=O)c1 ; O=C1CCCN1c1cncc(Nc2ncc(Cl)c(-c3cnn(C4CCCC4)c3)n2)c1]

Synthesis of 2,5-dichloro-4-(1-cyclopentylpyrazol-4-yl)pyrimidine (133-2). To a solution of 1-cyclopentyl-4-(4,4,5,5-tetramethyl-1,3,2-dioxaborolan-2-yl)pyrazole (133-1) (400 mg, 1.53 mmol, 1.00 eq) and 2,4,5-trichloropyrimidine (307 mg, 1.68 mmol, 1.10 eq) in Dioxane (4 mL) and H₂O (0.8 mL) was added Na₂CO₃ (485 mg, 4.58 mmol, 3.00 eq) and Pd(dppf)Cl₂ (111 mg, 152 umol, 0.100 eq) at 25° C. under N₂. The mixture was stirred at 80° C. for 4 h. LCMS showed the desired mass was detected. H₂O 5 mL was added to the mixture, and extracted with EtOAc (5 mL*3). The combined organic layers were dried over Na₂SO₄, filtered and concentrated under reduced pressure. The residue was purified by prep-TLC (SiO₂, Petroleum ether/Ethyl acetate=5/1, R_{f}=0.35) to give the title compound (133-2) (250 mg, 882 umol, 57.8% yield) as a yellow oil. LCMS (ES⁺): m/z 283.0 [M+H]⁺.

Synthesis of 1-[5-[[5-chloro-4-(1-cyclopentylpyrazol-4-yl)pyrimidin-2-yl]amino]-3-pyridyl]pyrrolidin-2-one (Compound 138). A mixture of 2,5-dichloro-4-(1-cyclopentylpyrazol-4-yl)pyrimidine (133-2) (100 mg, 353 umol, 1.00 eq), 1-(5-amino-3-pyridyl)pyrrolidine-2-one (68.8 mg, 388 umol, 1.10 eq), Cs₂CO₃ (287 mg, 882 umol, 2.50 eq), BINAP (21.9 mg, 35.3 umol, 0.100 eq) in dioxane (2 mL) and then the Pd₂(dba)₃ (32.3 mg, 35.3 umol, 0.100 eq) was added at 25° C. under N₂. The mixture was stirred at 100° C. for 12 h. LCMS showed the desired mass was detected. H₂O 10 mL was added to the mixture, and extracted with EtOAc (10 mL*3). The combined organic layers were dried over Na₂SO₄, filtered and concentrated under reduced pressure. The residue was purified by prep-HPLC (column: Phenomenex luna C18 80*40 mm*3 um; mobile phase: [water (FA)-ACN]; B %: 20%-60%, 8 min) to give the title compound (Compound 138) (30.2 mg, 68.0 μmol, 19.2% yield, 95.4% purity) as a white solid. LCMS (ES⁺): m/z 424.1 [M+H]⁺.

### Example 134. Synthesis of 1-[5-[[5-chloro-4-(1-cyclohexylpyrazol-4-yl)pyrimidin-2-yl]amino]-3-pyridyl]pyrrolidin-2-one (Compound 139)

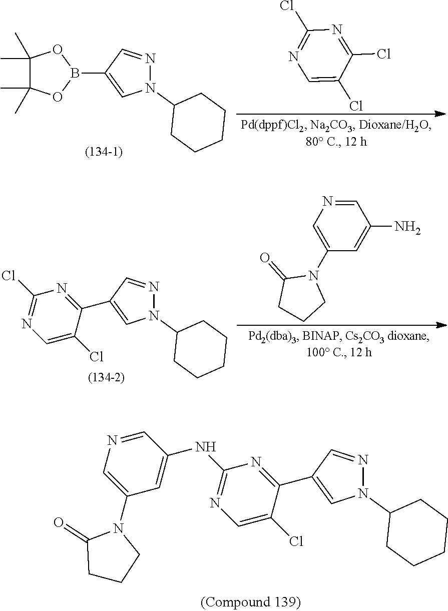 [SMILES: CC1(C)OB(c2cnn(C3CCCCC3)c2)OC1(C)C ; Clc1ncc(Cl)c(Cl)n1 ; Clc1ncc(Cl)c(-c2cnn(C3CCCCC3)c2)n1 ; Nc1cncc(N2CCCC2=O)c1 ; O=C1CCCN1c1cncc(Nc2ncc(Cl)c(-c3cnn(C4CCCCC4)c3)n2)c1]

Synthesis of 2,5-dichloro-4-(1-cyclohexylpyrazol-4-yl)pyrimidine (134-2). To a solution of 1-cyclohexyl-4-(4,4,5,5-tetramethyl-1,3,2-dioxaborolan-2-yl)pyrazole (134-1) (400 mg, 1.45 mmol, 1.00 eq) and 2,4,5-trichloropyrimidine (292 mg, 1.59 mmol, 1.10 eq) in Dioxane (4 mL) and H₂O (0.8 mL) was added Na₂CO₃ (460 mg, 4.34 mmol, 3.00 eq) and Pd(dppf)Cl₂ (105 mg, 144 μmol, 0.100 eq) at 25° C. The mixture was stirred at 80° C. for 12 h under N₂. LCMS showed the desired mass was detected. H₂O 5 mL was added to the mixture, and extracted with EtOAc (5 mL*3). The combined organic layers were dried over Na₂SO₄, filtered and concentrated under reduced pressure. The residue was purified by prep-TLC (SiO₂, Petroleum ether/Ethyl acetate=5/1, R_{f}=0.39) to give the title compound (134-2) (250 mg, 841 umol, 58.0% yield) as a yellow oil. LCMS (ES⁺): m/z 297.0 [M+H]⁺.

Synthesis of 1-[5-[[5-chloro-4-(1-cyclohexylpyrazol-4-yl)pyrimidin-2-yl]amino]-3-pyridyl]pyrrolidine-2-one (Compound 139). To a solution of 2,5-dichloro-4-(1-cyclohexylpyrazol-4-yl)pyrimidine (134-2) (100 mg, 336 umol, 1.00 eq), 1-(5-amino-3-pyridyl)pyrrolidine-2-one (65.6 mg, 370 umol, 1.10 eq), Cs₂CO₃ (274 mg, 841 umol, 2.50 eq), BINAP (20.9 mg, 33.6 umol, 0.100 eq) in dioxane (1 mL) was added Pd₂(dba)₃ (30.8 mg, 33.6 umol, 0.100 eq) at 25° C. under N₂. The mixture was stirred at 100° C. for 12 h. LCMS showed the desired mass was detected. H₂O 10 mL was added to the mixture, and extracted with EtOAc (10 mL*3). The combined organic layers were dried over Na₂SO₄, filtered and concentrated under reduced pressure. The residue was purified by prep-HPLC (column: Phenomenex luna C18 80*40 mm*3 um; mobile phase: [water (FA)-ACN]; B %: 25%-60%, 8 min) to give the title compound (Compound 139) (9.00 mg, 20.4 umol, 6.06% yield, 99.2% purity) as a white solid. LCMS (ES⁺): m/z 438.1 [M+H]⁺.

### Example 135. Synthesis of 1-(5-((5-chloro-4-(1-phenyl-1H-pyrazol-4-yl)pyrimidin-2-yl)amino)pyridin-3-yl)pyrrolidin-2-one (Compound 140)

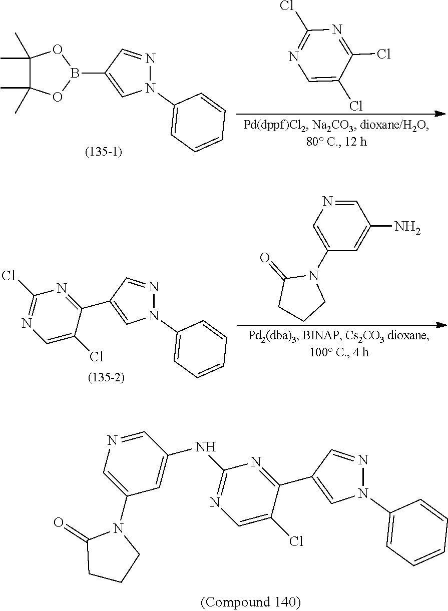 [SMILES: CC1(C)OB(c2cnn(-c3ccccc3)c2)OC1(C)C ; Clc1ncc(Cl)c(Cl)n1 ; Clc1ncc(Cl)c(-c2cnn(-c3ccccc3)c2)n1 ; Nc1cncc(N2CCCC2=O)c1 ; O=C1CCCN1c1cncc(Nc2ncc(Cl)c(-c3cnn(-c4ccccc4)c3)n2)c1]

Synthesis of 2,5-dichloro-4-(1-phenyl-1H-pyrazol-4-yl)pyrimidine (135-2). To a solution of 1-phenyl-4-(4,4,5,5-tetramethyl-1,3,2-dioxaborolan-2-yl)-1H-pyrazole (135-1)(400 mg, 1.48 mmol, 1.00 eq) and 2,4,5-trichloropyrimidine (271 mg, 1.48 mmol, 1.00 eq) in dioxane (1 mL) and H₂O (0.200 mL) were added Na₂CO₃ (470 mg, 4.44 mmol, 3.00 eq) and Pd(dppf)Cl₂ (216 mg, 296 umol, 0.200 eq) under N₂ at 25° C. The mixture was stirred at 80° C. for 12 h. LCMS showed the desired mass was detected. The reaction mixture was diluted with H₂O 10 mL and extracted with EtOAc (10 mL*3). The combined organic layers were dried over Na₂SO₄, filtered and concentrated under reduced pressure. The residue was purified by column chromatography (SiO₂, Petroleum ether/Ethyl acetate=20/1 to 1/1) to give the title compound (135-2) (110 mg, 315 umol, 21.3% yield, 83.5% purity) as a white solid. LCMS (ES⁺): m/z 291.2 [M+H]⁺.

Synthesis of 1-(5-((5-chloro-4-(1-phenyl-1H-pyrazol-4-yl)pyrimidin-2-yl)amino)pyridin-3-yl)pyrrolidin-2-one (Compound 140). To a solution of 2,5-dichloro-4-(1-phenyl-1H-pyrazol-4-yl)pyrimidine (135-2) (90.0 mg, 309 umol, 1.00 eq) and 1-(5-aminopyridin-3-yl)pyrrolidin-2-one (54.7 mg, 309 umol, 1.00 eq) in dioxane (5 mL) were added Cs₂CO₃ (251 mg, 772 umol, 2.50 eq) and BINAP (19.2 mg, 30.9 umol, 0.100 eq) and Pd₂(dba)₃ (28.3 mg, 30.9 umol, 0.100 eq) at 20° C. under N₂. The mixture was stirred at 100° C. for 4 h. LCMS showed the desired mass was detected. The residue was diluted with H₂O 2 mL and extracted with EtOAc (2 mL*3). The combined organic layers were dried over Na₂SO₄ filtered and concentrated under reduced pressure. The residue was purified by prep-HPLC (column: Phenomenex luna C18 80*40 mm*3 um; mobile phase: [water (FA)-ACN]; B %: 20%-60%, 8 min) to give the title compound (Compound 140) (45.3 mg, 98.8 umol, 31.9% yield, 94.2% purity) as a white solid. LCMS (ES⁺): m/z 432.1 [M+H]⁺.

### Example 136. Synthesis of 1-(5-((5-chloro-4-(1-phenylpiperidin-3-yl)pyrimidin-2-yl)amino)pyridin-3-yl)pyrrolidin-2-one (Compound 141)

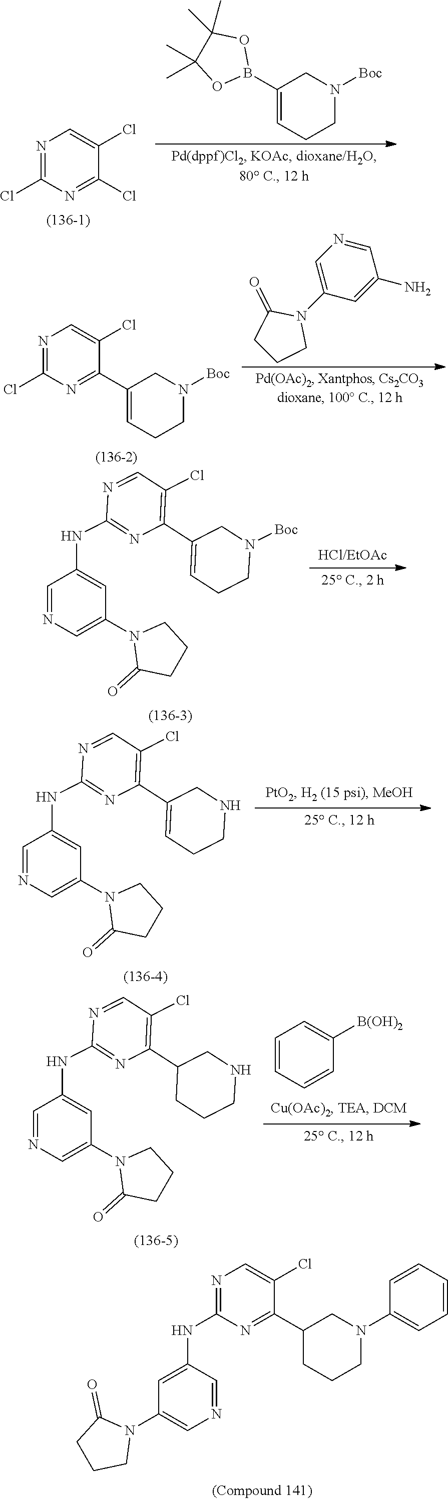

Synthesis of tert-butyl 5-(2,5-dichloropyrimidin-4-yl)-3,6-dihydropyridine-1(2H)-carboxylate (136-2). Two batches in parallel were setup. To a solution of 2,4,5-trichloropyrimidine (136-1) (5.00 g, 27.3 mmol, 1.00 eq) and tert-butyl 5-(4,4,5,5-tetramethyl-1,3,2-dioxaborolan-2-yl)-3,6-dihydropyridine-1(2H)-carboxylate (9.27 g, 29.9 mmol, 1.10 eq) in dioxane (50 mL) and H₂O (10 mL) was added Pd(dppf)Cl₂ (1.99 g, 2.73 mmol, 0.100 eq) and KOAc (8.03 g, 81.7 mmol, 3.00 eq) at 25° C., The mixture was stirred at 80° C. for 12 h under N₂. LCMS showed the desired mass was detected. Two batches in parallel were combined. The reaction mixture was diluted with water 50 mL and extracted with EtOAc (50 mL*3). The combined organic layers were washed with brine (50 mL*3), dried over Na₂SO₄, filtered and concentrated under reduced pressure. The residue was purified by column chromatography (SiO₂, Petroleum ether/Ethyl acetate=1/0 to 1/1) to give the title compound (136-2) (15.0 g, 29.3 mmol, 53.9% yield, 64.7% purity) as a yellow solid. LCMS (ES⁺): m/z 274.2 [M+H−56]⁺

Synthesis of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (136-3). A mixture of tert-butyl 5-(2,5-dichloropyrimidin-4-yl)-3,6-dihydropyridine-1(2H)-carboxylate (136-2) (6.00 g, 18.1 mmol, 1.00 eq), 1-(5-aminopyridin-3-yl)pyrrolidin-2-one (4.51 g, 25.4 mmol, 1.40 eq), Pd(OAc)₂ (407 mg, 1.82 mmol, 0.100 eq), Xantphos (1.05 g, 1.82 mmol, 0.100 eq) and Cs₂CO₃ (17.7 g, 54.5 mmol, 3.00 eq) in dioxane (20 mL) at 25° C. was degassed and purged with N₂ for 3 times, and then the mixture was stirred at 100° C. for 12 h under N₂ atmosphere. LCMS showed the desired mass was detected. The reaction mixture was diluted with H₂O 20 mL at 25° C., and extracted with EtOAc (15 mL*3). The combined organic layers were washed with brine (20 mL), dried over Na₂SO₄, filtered and concentrated under reduced pressure. The residue was purified by column chromatography (SiO₂, Petroleum ether/Ethyl acetate=1/0 to 0/1) to give the title compound (136-3) (3.65 g, 2.91 mmol, 16.0% yield, 37.5% purity) as a yellow solid. LCMS (ES⁺): m/z 471.3 [M+H]⁺.

Synthesis of 1-(5-((5-chloro-4-(1,2,5,6-tetrahydropyridin-3-yl)pyrimidin-2-yl)amino)pyridin-3-yl)pyrrolidin-2-one (136-4). A solution of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (136-3) (3.00 g, 6.37 mmol, 1.00 eq) in HCl/EtOAc (4 M, 20 mL) was stirred at 25° C. for 2 h. LCMS showed the desired mass was detected. The reaction mixture was filtered and the filtrate concentrated under high vacuum to give the title compound (136-4) (3.00 g, crude) as a yellow solid. LCMS (ES⁺): m/z 371.0 [M+H]⁺.

Synthesis of 1-(5-((5-chloro-4-(piperidin-3-yl)pyrimidin-2-yl)amino)pyridin-3-yl)pyrrolidin-2-one (136-5). A mixture of 1-(5-((5-chloro-4-(1,2,5,6-tetrahydropyridin-3-yl)pyrimidin-2-yl)amino)pyridin-3-yl)pyrrolidin-2-one (136-4) (100 mg, 269 umol, 1.00 eq) in MeOH (10 mL) was added PtO₂ (100 mg, 440 umol, 1.63 eq) at 25° C. under N₂ atmosphere. The suspension was degassed and purged with H₂ for 3 times. The mixture was stirred under H₂ (15 Psi) at 25° C. for 12 h. LCMS showed the desired mass was detected. The reaction mixture was filtered and the filtrate concentrated under high vacuum. The residue was purified by prep-HPLC (column: Waters Xbridge BEH C18 250*70 mm*10 um; mobile phase: [water (NH₄HCO₃)-ACN]; B %: 15%-45%, 20 min) to give the title compound (136-5) (143 mg, 147 umol, 10.9% yield, 38.5% purity) as a white solid. LCMS (ES⁺): m/z 373.3 [M+H]⁺.

Synthesis of 1-(5-((5-chloro-4-(1-phenylpiperidin-3-yl)pyrimidin-2-yl)amino)pyridin-3-yl)pyrrolidin-2-one (Compound 141). To a solution of 1-(5-((5-chloro-4-(piperidin-3-yl)pyrimidin-2-yl)amino)pyridin-3-yl)pyrrolidin-2-one (136-5) (68.0 mg, 182 umol, 1.00 eq) in DCM (3 mL) was added phenylboronic acid (22.2 mg, 182 umol, 1.00 eq), Cu(OAc)₂, (16.5 mg, 91.1 umol, 0.500 eq) and TEA (55.3 mg, 547 umol, 76.1 uL, 3.00 eq) at 25° C. The mixture was stirred at 25° C. for 12 h. LCMS showed the desired mass was detected. The reaction mixture was diluted with H₂O 3 ml at 25° C., and extracted with DCM (3 mL*3). The combined organic layers were washed with brine (3 mL), dried over Na₂SO₄, filtered and concentrated under reduced pressure. The residue was purified by prep-HPLC (column: Waters Xbridge Prep OBD C18 150*40 mm*10 um; mobile phase: [water (NH₃H₂O+NH₄HCO₃)-ACN]; B %: 40%-70%, 8 min) to give the title compound (Compound 141) (2.70 mg, 5.64 μmol, 3.09% yield, 93.7% purity) as a white solid. LCMS (ES⁺): m/z 449.3 [M+H]⁺.

### Example 137. Synthesis of 1-(5-((5-chloro-4-(1-phenyl-1H-pyrazol-3-yl)pyrimidin-2-yl)amino)pyridin-3-yl)pyrrolidine-2-one (Compound 143)

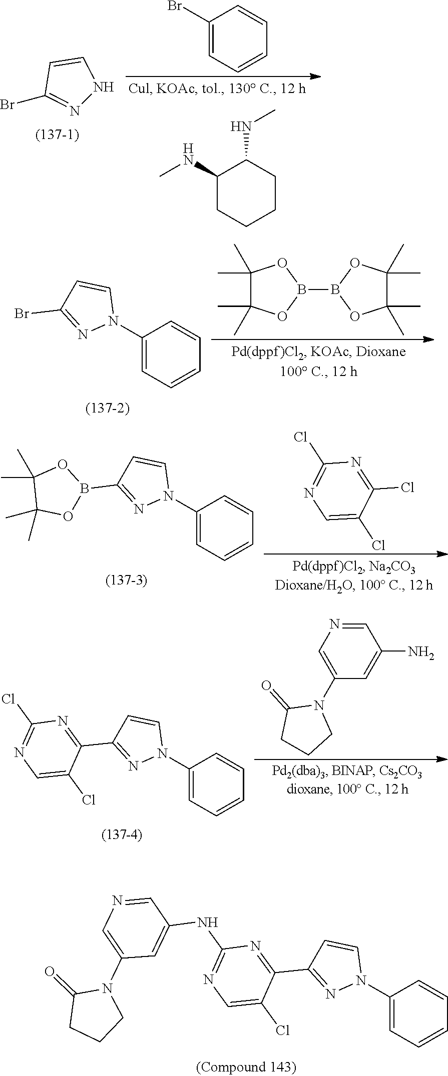

Synthesis of 3-bromo-1-phenyl-1H-pyrazole (137-2). To a solution of 3-bromo-1H-pyrazole (137-1) (2.00 g, 13.6 mmol, 1.00 eq) in toluene (30.0 mL) were added KOAc (5.34 g, 54.4 mmol, 4.00 eq), (1R,2R)—N₁,N₂-dimethylcyclohexane-1,2-diamine (387.1 mg, 2.72 mmol, 0.200 eq), bromobenzene (6.41 g, 40.8 mmol, 4.30 mL, 3.00 eq) and CuI (129 mg, 680 umol, 0.0500 eq) at 25° C. The mixture was stirred at 130° C. for 12 h. LCMS showed 3-bromo-1H-pyrazole (137-1) was consumed completely and the desired mass was detected. Water (50.0 mL) was added to the solution, the mixture was extracted with ethyl acetate (50.0 mL*3), the combined organic layers were dried over Na₂SO₄, and concentrated under reduced pressure. The residue was purified by column chromatography (SiO₂, Petroleum ether/Ethyl acetate=10/1 to 8/1) to give the title compound (137-2) (670 mg, 2.61 mmol, 19.2% yield, 86.8% purity) as a white solid. LCMS (ES⁺): m/z 223.2 [M+H]⁺.

Synthesis of 1-phenyl-3-(4,4,5,5-tetramethyl-1,3,2-dioxaborolan-2-yl)-1H-pyrazole (137-3). To a solution of 3-bromo-1-phenyl-1H-pyrazole (137-2) (500 mg, 2.24 mmol, 1.00 eq) in dioxane (10.0 mL) was added KOAc (329 mg, 3.36 mmol, 1.50 eq), 4,4,5,5-tetramethyl-2-(4,4,5,5-tetramethyl-1,3,2-dioxaborolan-2-yl)-1,3,2-dioxaborolane (1.14 g, 4.48 mmol, 2.00 eq) and Pd(dppf)Cl₂ (328 mg, 448 umol, 0.200 eq) under N₂ at 25° C. The mixture was stirred at 100° C. for 12 h. LCMS showed 3-bromo-1-phenyl-1H-pyrazole (137-2) was consumed completely and desired mass was detected. Water (50.0 mL) was added to the solution, the mixture was extracted with ethyl acetate (50.0 mL*3), the combined organic layers were dried over Na₂SO₄, and concentrated under reduced pressure. The residue was purified by column chromatography by prep-TLC (SiO₂, PE/EA=5/1) to give the title compound (137-3) (350 mg, 1.47 mmol, 65.7% yield, 79.1% purity) as a white solid. LCMS (ES⁺): m/z 189.3 [M+H−82]⁺ (boric acid)

Synthesis of 2,5-dichloro-4-(1-phenyl-1H-pyrazol-3-yl)pyrimidine (137-4). To a solution of 1-phenyl-3-(4,4,5,5-tetramethyl-1,3,2-dioxaborolan-2-yl)-1H-pyrazole (137-3) (300 mg, 1.60 mmol, 1.00 eq) in dioxane (5.00 mL) and H₂O (1.00 mL) was added Pd(dppf)Cl₂ (233 mg, 319 umol, 0.200 eq), 2,4,5-trichloropyrimidine (292 mg, 1.60 mmol, 1.00 eq) and Na₂CO₃ (253 mg, 2.39 mmol, 1.50 eq) at 25° C., The mixture was stirred at 100° C. for 12 h. LCMS showed 1-phenyl-3-(4,4,5,5-tetramethyl-1,3,2-dioxaborolan-2-yl)-1H-pyrazole (137-3) was consumed completely and the desired mass was detected. Water (50.0 mL) was added to the solution, the mixture was extracted with ethyl acetate (50.0 mL*3), the combined organic layers were dried over Na₂SO₄, and concentrated under reduced pressure. The residue was purified by column chromatography by prep-TLC (SiO₂, PE/EA=8/1) to give the title compound (137-4) (250 mg, 721 umol, 45.2% yield, 84.0% purity) as a white solid. LCMS (ES⁺): m/z 290.9 [M+H]⁺.

Synthesis of 1-(5-((5-chloro-4-(1-phenyl-JH-pyrazol-3-yl)pyrimidin-2-yl)amino)pyridin-3-yl)pyrrolidine-2-one (Compound 143). To a solution of 2,5-dichloro-4-(1-phenyl-1H-pyrazol-3-yl)pyrimidine (137-4) (121 mg, 686 umol, 1.00 eq) in dioxane (2.00 mL) was added BINAP (85.5 mg, 137 umol, 0.200 eq), 2,5-dichloro-4-(1-phenylpyrazol-3-yl)pyrimidine (200 mg, 686 umol, 1.00 eq), Pd₂(dba)₃ (125 mg, 137 umol, 0.200 eq) and Cs₂CO₃ (671 mg, 2.06 mmol, 3.00 eq) at 25° C. in N₂. The mixture was stirred at 100° C. for 12 h. LCMS showed 2,5-dichloro-4-(1-phenyl-1H-pyrazol-3-yl)pyrimidine (137-4) was consumed completely and the desired mass was detected. Water (50.0 mL) was added to the solution, the mixture was extracted with ethyl acetate (50.0 mL*3), the combined organic layers were dried over Na₂SO₄, and concentrated under reduced pressure. The residue was purified by prep-HPLC (column: Phenomenex Luna C18 200*40 mm*10 um; mobile phase: [water (FA)-ACN]; B %: 20%-50%, 8 min) to give the title compound (Compound 143) (28.2 mg, 63.3 μmol, 9.22% yield, 96.9% purity) as white solid. LCMS (ES⁺): m/z 432.1 [M+H]⁺.

### Example 138. Synthesis of 1-(5-((5-chloro-4-(1-cyclopentyl-1H-pyrazol-3-yl)pyrimidin-2-yl)amino)pyridin-3-yl)pyrrolidin-2-one (Compound 144)

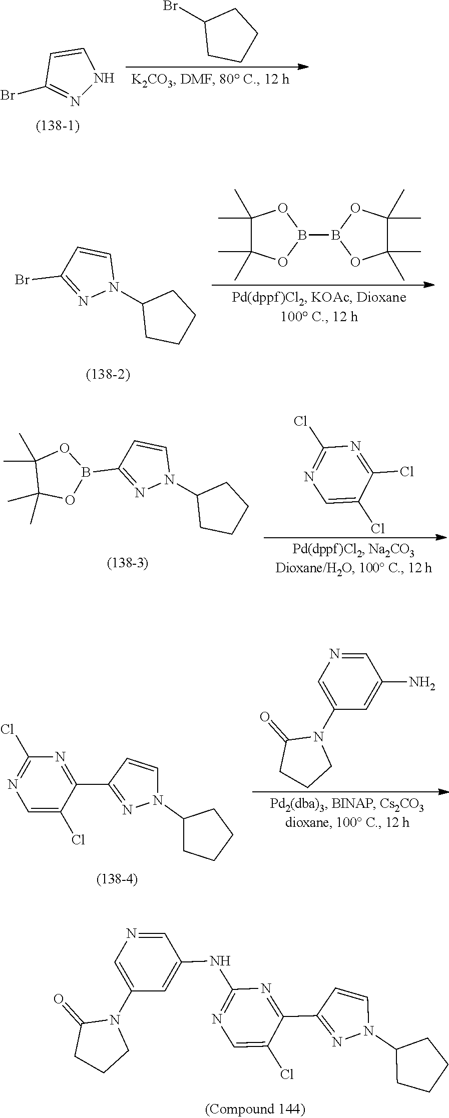

Synthesis of 3-bromo-1-cyclopentyl-1H-pyrazole (138-2). To a solution of 3-bromo-1H-pyrazole (138-1) (2.00 g, 13.6 mmol, 1.00 eq) in DMF (2 mL) were added bromocyclopentane (6.08 g, 40.8 mmol, 4.38 mL, 3.00 eq) and K₂CO₃ (3.76 g, 27.2 mmol, 2.00 eq) at 25° C. The mixture was stirred at 80° C. for 12 h. LCMS showed the desired mass was detected. The reaction mixture was diluted with H₂O 40 mL and extracted with EtOAc (30 mL*3). The combined organic layers were washed with aqueous NaCl 30 mL, dried over Na₂SO₄, filtered and concentrated under reduced pressure. The residue was purified by column chromatography (SiO₂, Petroleum ether/Ethyl acetate=10/1 to 5/1) to give the title compound (138-2) (800 mg, 3.46 mmol, 25.4% yield, 93.1% purity) as a yellow oil. LCMS (ES⁺): m/z 215.0 [M+H]⁺.

Synthesis of 1-cyclopentyl-3-(4,4,5,5-tetramethyl-1,3,2-dioxaborolan-2-yl)-1H-pyrazole (138-3). A mixture of 3-bromo-1-cyclopentyl-1H-pyrazole (138-2) (750 mg, 3.49 mmol, 1.00 eq), 4,4,5,5-tetramethyl-2-(4,4,5,5-tetramethyl-1,3,2-dioxaborolan-2-yl)-1,3,2-dioxaborolane (1.77 g, 6.97 mmol, 2.00 eq), KOAc (513 mg, 5.23 mmol, 1.50 eq) and Pd(dppf)Cl₂ (255 mg, 348 umol, 0.100 eq) in dioxane (10 mL) was degassed and purged with N₂ for 3 times, and then the mixture was stirred at 100° C. for 12 h under N₂ atmosphere. LCMS showed the desired mass was detected. The reaction mixture was diluted with H₂O 40 mL and extracted with EA (30 mL*3). The combined organic layers were washed with brine 30 mL, dried over Na₂SO₄, filtered and concentrated under reduced pressure. The residue was purified by prep-TLC (SiO₂, Dichloromethane/Methanol=10/1 to 5/1) to give the title compound (138-3) (1.10 g, 1.76 mmol, 50.6% yield, 42.0% purity) as a brown oil. LCMS (ES⁺): m/z 181.3 [M+H−82]⁺ (boric acid).

Synthesis of 2,5-dichloro-4-(1-cyclopentyl-1H-pyrazol-3-yl)pyrimidine (138-4). A mixture of 1-cyclopentyl-3-(4,4,5,5-tetramethyl-1,3,2-dioxaborolan-2-yl)-1H-pyrazole (138-3) (300 mg, 1.14 mmol, 1.00 eq), 2,4,5-trichloropyrimidine (209 mg, 1.14 mmol, 1.00 eq), Na₂CO₃ (242 mg, 2.29 mmol, 2.00 eq), Pd(dppf)Cl₂ (83.7 mg, 114 umol, 0.100 eq) in dioxane (5 mL) and H₂O (1 mL) was degassed and purged with N₂ for 3 times, and then the mixture was stirred at 100° C. for 12 h under N₂ atmosphere. LCMS showed the desired mass was detected. The reaction mixture was diluted with H₂O 40 mL and extracted with EA (30 mL*3). The combined organic layers were washed with brine 30 mL, dried over Na₂SO₄, filtered and concentrated under reduced pressure. The residue was purified by prep-TLC (SiO₂, Petroleum ether/Ethyl acetate=8/1) to give the title compound (138-4) (80.0 mg, 276 μmol, 24.1% yield, 97.7% purity) as a yellow solid. LCMS (ES⁺): m/z 283.0 [M+H]⁺.

Synthesis of 1-(5-((5-chloro-4-(I-cyclopentyl-1H-pyrazol-3-yl)pyrimidin-2-yl)amino)pyridin-3-yl)pyrrolidin-2-one (Compound 144). A mixture of 2,5-dichloro-4-(1-cyclopentyl-1H-pyrazol-3-yl)pyrimidine (138-4) (70.0 mg, 247 μmol, 1.00 eq), 1-(5-amino-3-pyridyl)pyrrolidin-2-one (56.9 mg, 321 μmol, 1.30 eq), Pd₂(dba)₃ (22.6 mg, 24.7 μmol, 0.100 eq), BINAP (15.3 mg, 24.7 μmol, 0.100 eq) and Cs₂CO₃ (241 mg, 741 μmol, 3.00 eq) in dioxane (2 mL) was degassed and purged with N₂ for 3 times at 25° C., and then the mixture was stirred at 100° C. for 12 h under N₂ atmosphere. LCMS showed the desired mass was detected. The reaction mixture was diluted with H₂O 10 mL and extracted with EtOAc (15 mL*3). The combined organic layers were washed with aqueous NaCl 10 mL, dried over Na₂SO₄, filtered and concentrated under reduced pressure. The residue was purified by prep-HPLC (column: Phenomenex Luna C18 75*30 mm*3 um; mobile phase: [water (FA)-ACN]; B %: 15%-55%, 8 min) to give the title compound (Compound 144) (21.0 mg, 47.6 μmol, 19.2% yield, 96.2% purity) as a white solid. LCMS (ES⁺): m/z 424.2 [M+H]⁺.

### Example 139. Synthesis of 1-(5-((5-chloro-4-(1-cyclobutylpiperidin-3-yl)pyrimidin-2-yl)amino)pyridin-3-yl)pyrrolidin-2-one (Compound 145)

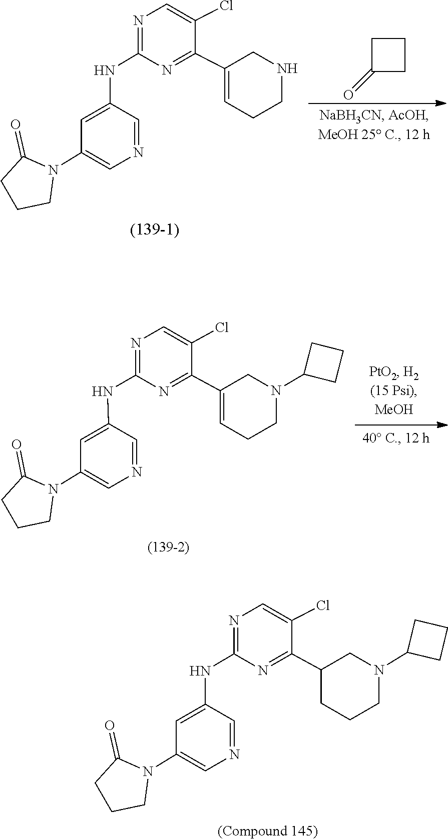 [SMILES: O=C1CCCN1c1cncc(Nc2ncc(Cl)c(C3=CCCNC3)n2)c1 ; O=C1CCC1 ; O=C1CCCN1c1cncc(Nc2ncc(Cl)c(C3=CCCN(C4CCC4)C3)n2)c1 ; O=C1CCCN1c1cncc(Nc2ncc(Cl)c(C3CCCN(C4CCC4)C3)n2)c1]

Synthesis of 1-(5-((5-chloro-4-(1-cyclobutyl-1,2,5,6-tetrahydropyridin-3-yl)pyrimidin-2-yl)amino)pyridin-3-yl)pyrrolidin-2-one (139-2). To a solution of 1-(5-((5-chloro-4-(1,2,5,6-tetrahydropyridin-3-yl)pyrimidin-2-yl)amino)pyridin-3-yl)pyrrolidin-2-one (139-1) (700 mg, 1.89 mmol, 1.00 eq) in MeOH (10 mL) was added cyclobutanone (264 mg, 3.78 mmol, 282 μL, 2.00 eq) and AcOH (1.13 g, 18.8 mmol, 1.08 mL, 10.0 eq) at 25° C., the mixture was stirred at 25° C. for 0.5 h, then NaBH₃CN (237 mg, 3.78 mmol, 2.00 eq) was added to the mixture at 25° C. The mixture was stirred at 25° C. for 11.5 h. LCMS showed the desired mass was detected. The reaction mixture was filtered and the filtrate concentrated under high vacuum. The residue was purified by prep-TLC (SiO₂, Dichloromethane/Methanol=10/1) to give the title compound (139-2) (313 mg, 642 μmol, 34.0% yield, 87.2% purity) as a white solid. LCMS (ES⁺): m/z 425.3 [M+H]⁺.

Synthesis of 1-(5-((5-chloro-4-(I-cyclobutylpiperidin-3-yl)pyrimidin-2-yl)amino)pyridin-3-yl)pyrrolidin-2-one (Compound 145). A mixture of 1-(5-((5-chloro-4-(1-cyclobutyl-1,2,5,6-tetrahydropyridin-3-yl)pyrimidin-2-yl)amino)pyridin-3-yl)pyrrolidin-2-one (139-2) (313 mg, 736 μmol, 1.00 eq) in MeOH (8 mL) was added PtO₂ (260 mg, 1.15 mmol, 1.56 eq) at 25° C. under N₂ atmosphere. The suspension was degassed and purged with H₂ for 3 times. The mixture was stirred under H₂ (15 Psi) at 40° C. for 12 h. LCMS showed the desired mass was detected. The reaction mixture was filtered and the filtrate was concentrated under high vacuum. The residue was purified by prep-HPLC (column: Waters Xbridge Prep OBD C18 150*40 mm*10 um; mobile phase: [water (NH₃H₂O+NH₄HCO₃)-ACN]; B %: 25%-45%, 8 min) to give the title compound (Compound 145) (17.1 mg, 40.0 μmol, 5.44% yield, 100% purity) as a white solid. LCMS (ES⁺): m/z 427.2 [M+H]⁺.

### Example 140 1-(5-((4-(3-cyclopropylphenyl)-5-fluoropyrimidin-2-yl)amino)pyridin-2-yl)pyrrolidin-2-one (Compound 147)

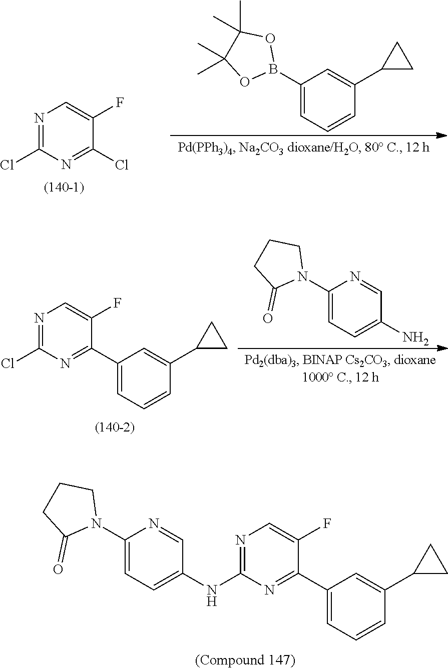 [SMILES: Fc1cnc(Cl)nc1Cl ; CC1(C)OB(c2cccc(C3CC3)c2)OC1(C)C ; Fc1cnc(Cl)nc1-c1cccc(C2CC2)c1 ; Nc1ccc(N2CCCC2=O)nc1 ; O=C1CCCN1c1ccc(Nc2ncc(F)c(-c3cccc(C4CC4)c3)n2)cn1]

Synthesis of 2-chloro-4-(3-cyclopropylphenyl)-5-fluoropyrimidine (140-2). To a solution of 2,4-dichloro-5-fluoro-pyrimidine (140-1) (400 mg, 2.40 mmol, 1.00 eq) and 2-(3-cyclopropylphenyl)-4,4,5,5-tetramethyl-1,3,2-dioxaborolane (702 mg, 2.87 mmol, 1.20 eq) in dioxane (5 mL) and H₂O (0.5 mL) were added Na₂CO₃ (508 mg, 4.79 mmol, 2.00 eq) and Pd(PPh₃)₄ (277 mg, 240 μmol, 0.100 eq) at 15° C. The mixture was stirred at 80° C. for 12 h under N₂. LCMS showed 2,4-dichloro-5-fluoro-pyrimidine (140-1) was consumed completely and the desired mass was detected. The mixture was concentrated under reduced pressure. The residue was purified by prep-TLC (SiO₂, Petroleum ether:Ethyl acetate=5:1) to give the title compound (140-2) (380 mg, 1.53 mmol, 63.8% yield) was obtained as a colourless oil. LCMS (ES⁺): m/z 249.0 [M+H]⁺.

Synthesis of 1-(5-((4-(3-cyclopropylphenyl)-5-fluoropyrimidin-2-yl)amino)pyridin-2-yl)pyrrolidin-2-one (Compound 147). To a solution of 2-chloro-4-(3-cyclopropylphenyl)-5-fluoro-pyrimidine (140-2) (150 mg, 603 μmol, 1.00 eq) and 1-(5-amino-2-pyridyl)pyrrolidin-2-one (107 mg, 603 μmol, 1.00 eq) in dioxane (4 mL) were added BINAP (37. 6 mg, 60.3 μmol, 0.100 eq), Pd₂(dba)₃ (55.2 mg, 60.3 μmol, 0.100 eq) and Cs₂CO₃ (393 mg, 1.21 mmol, 2.00 eq) at 15° C. The mixture was stirred at 100° C. for 12 h under N₂. LCMS showed 2-chloro-4-(3-cyclopropylphenyl)-5-fluoro-pyrimidine (140-2) was consumed completely and the desired mass was detected. The mixture was filtered and the filtrate was concentrated under reduced pressure. The residue was purified by prep-HPLC (column: Phenomenex Luna C18 200*40 mm*10 um; mobile phase: [water (FA)-ACN]; B %: 40%-70%, 8 min) to give title compound (Compound 147) (61.8 mg, 153 μmol, 25.3% yield, 96.2% purity) was obtained as a yellow solid. LCMS (ES⁺): m/z 390.0 [M+H]⁺.

### Example 141. Synthesis of 1-(5-((5-fluoro-4-(1-(oxetan-3-yl)-JH-pyrazol-4-yl)pyrimidin-2-yl)amino)pyridin-3-yl)pyrrolidin-2-one (Compound 148)

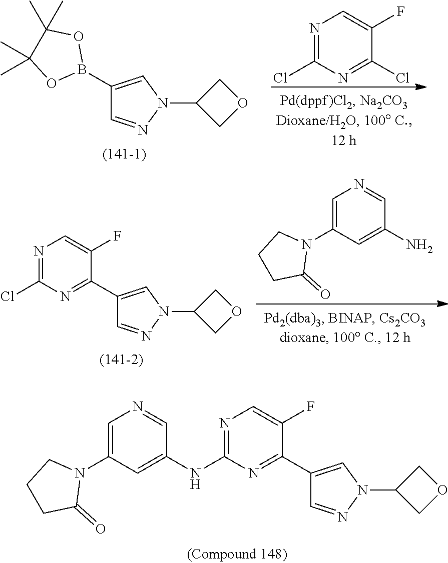 [SMILES: CC1(C)OB(c2cnn(C3COC3)c2)OC1(C)C ; Fc1cnc(Cl)nc1Cl ; Fc1cnc(Cl)nc1-c1cnn(C2COC2)c1 ; Nc1cncc(N2CCCC2=O)c1 ; O=C1CCCN1c1cncc(Nc2ncc(F)c(-c3cnn(C4COC4)c3)n2)c1]

Synthesis of 2-chloro-5-fluoro-4-(1-(oxetan-3-yl)-1H-pyrazol-4-yl)pyrimidine (141-2). To a solution of 1-(oxetan-3-yl)-4-(4,4,5,5-tetramethyl-1,3,2-dioxaborolan-2-yl)-1H-pyrazole (141-1) (1.00 g, 4.00 mmol, 1.00 eq) and 2,4-dichloro-5-fluoro-pyrimidine (801 mg, 4.80 mmol, 1.20 eq) in dioxane (20 mL) and H₂O (4 mL) was added Pd(dppf)Cl₂ (292 mg, 399 μmol, 0.100 eq) and Na₂CO₃ (1.27 g, 12.0 mmol, 3.00 eq) at 20° C. The mixture was stirred at 100° C. for 12 h under N₂ atmosphere. LCMS showed 1-(oxetan-3-yl)-4-(4,4,5,5-tetramethyl-1,3,2-dioxaborolan-2-yl)pyrazole (141-1) was consumed completely and the desired mass was detected. The reaction mixture was diluted with H₂O 10 mL, and extracted with EtOAc (10 mL*3). The combined organic layers were washed with sat. aq. NaCl (10 mL), dried over Na₂SO₄, filtered and concentrated under reduced pressure. The residue was purified by column chromatography (SiO₂, Petroleum ether/Ethyl acetate=0/1 to 0/1) to give the title compound (141-2) (680 mg, 2.56 mmol, 64.1% yield, 96.0% purity) as a yellow solid. LCMS (ES⁺): m/z 255.0 [M+H]⁺.

Synthesis of 1-(5-((5-fluoro-4-(1-(oxetan-3-yl)-1H-pyrazol-4-yl)pyrimidin-2-yl)amino)pyridin-3-yl)pyrrolidin-2-one (Compound 148). To a solution of 2-chloro-5-fluoro-4-[1-(oxetan-3-yl)pyrazol-4-yl]pyrimidine (141-2) (200 mg, 785 μmol, 1.00 eq) and 1-(5-amino-3-pyridyl)pyrrolidin-2-one (167 mg, 942 μmol, 1.20 eq) in dioxane (10 mL) was added BINAP (48.9 mg, 78.5 μmol, 0.100 eq) and Pd₂(dba)₃ (71.9 mg, 78.5 μmol, 0.100 eq) and Cs₂CO₃ (767 mg, 2.36 mmol, 3.00 eq) at 20° C. The mixture was stirred at 100° C. for 12 h under N₂ atmosphere. LCMS showed 2-chloro-5-fluoro-4-[1-(oxetan-3-yl)pyrazol-4-yl]pyrimidine (141-2) was consumed completely and the desired mass was detected. The reaction mixture was filtered and the filtrate was concentrated under high vacuum. The residue was purified by prep-HPLC. (Phenomenex Luna C18 200*40 mm*10 um; mobile phase: [water (FA)-ACN]; B %: 5%-35%, 8 min) to give the title compound (Compound 148) (32.1 mg, 81.1 μmol, 10.3% yield) as a white solid. LCMS (ES⁺): m/z 396.1 [M+H]⁺.

### Example 142. Synthesis of 1-(5-((5-chloro-4-(1-cyclopropylpiperidin-3-yl)pyrimidin-2-yl)amino)pyridin-3-yl)pyrrolidin-2-one (Compound 149)

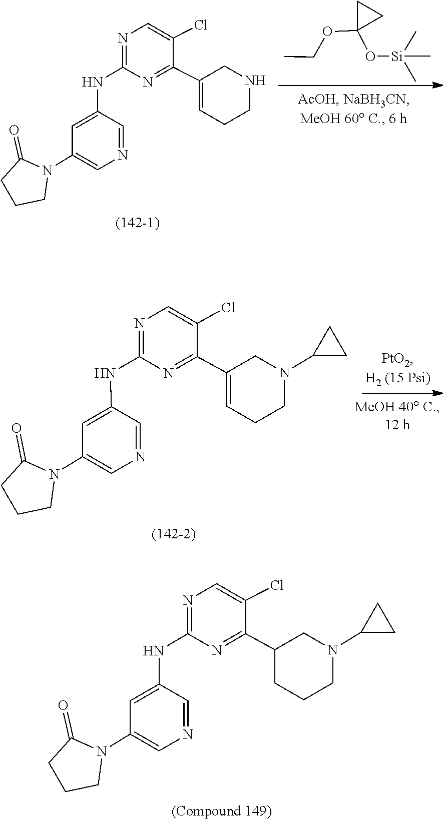

Synthesis of 1-(5-((5-chloro-4-(1-cyclopropyl-1,2,5,6-tetrahydropyridin-3-yl)pyrimidin-2-yl)amino)pyridin-3-yl)pyrrolidin-2-one (142-2). To a solution of 1-(5-((5-chloro-4-(1,2,5,6-tetrahydropyridin-3-yl)pyrimidin-2-yl)amino)pyridin-3-yl)pyrrolidin-2-one (142-1) (600 mg, 1.62 mmol, 1.00 eq) in MeOH (8 mL) were added (1-ethoxycyclopropoxy)trimethylsilane (1.23 g, 7.04 mmol, 1.42 mL, 4.35 eq), AcOH (844 mg, 14.0 mmol, 804 μL, 8.69 eq) at 25° C., the mixture was stirred at 25° C. for 0.5 h, then NaBH₃CN (472 mg, 7.52 mmol, 4.65 eq) was added to the mixture at 25° C. The mixture was stirred at 60° C. for 5.5 h. LC-MS showed the desired mass was detected. The reaction mixture was quenched by addition H₂O 10 ml at 25° C., and extracted with EtOAc (10 mL*3). The combined organic layers were washed with brine (10 mL*1), dried over Na₂SO₄, filtered and concentrated under reduced pressure. The residue was purified by prep-TLC (SiO₂, Dichloromethane/Methanol=10/1) to give the title compound (142-2) (214 mg, 476 μmol, 29.4% yield, 91.4% purity) as a white solid. LCMS (ES⁺): m/z 411.2 [M+H]⁺.

Synthesis of 1-(5-((5-chloro-4-(1-cyclopropylpiperidin-3-yl)pyrimidin-2-yl)amino)pyridin-3-yl)pyrrolidine-2-one (Compound 149). A mixture of 1-(5-((5-chloro-4-(1-cyclopropyl-1,2,5,6-tetrahydropyridin-3-yl)pyrimidin-2-yl)amino)pyridin-3-yl)pyrrolidine-2-one (142-2) (80.0 mg, 194 μmol, 1.00 eq) in MeOH (4 mL) was added PtO₂ (68.9 mg, 303 μmol, 1.56 eq) at 25° C. under N₂ atmosphere. The suspension was degassed and purged with H₂ for 3 times. The mixture was stirred under H₂ (15 Psi) at 40° C. for 12 h. LCMS showed the desired mass was detected. The reaction mixture was filtered and the filtrate concentrated under high vacuum. The residue was purified by prep-HPLC (column: Phenomenex Luna C18 200*40 mm*10 um; mobile phase: [water (FA)-ACN]; B %: 1%-35%, 8 min) to give the title compound (Compound 149) (5.10 mg, 11.1 μmol, 5.72% yield, 98.7% purity, 0.85FA) as a white solid. LCMS (ES⁺): m/z 413.1 [M+H]⁺.

### Example 143. 1-(2-((5-chloro-4-(3-cyclohexylphenyl)pyrimidin-2-yl)amino)pyridin-4-yl)pyrrolidin-2-one (Compound 150)

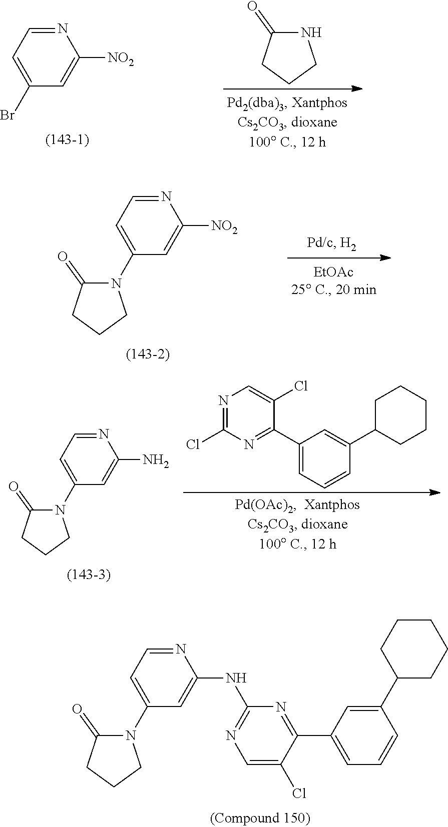 [SMILES: O=[N+]([O-])c1cc(Br)ccn1 ; O=C1CCCN1 ; O=C1CCCN1c1ccnc([N+](=O)[O-])c1 ; Nc1cc(N2CCCC2=O)ccn1 ; Clc1ncc(Cl)c(-c2cccc(C3CCCCC3)c2)n1 ; O=C1CCCN1c1ccnc(Nc2ncc(Cl)c(-c3cccc(C4CCCCC4)c3)n2)c1]

Synthesis of 1-(2-nitropyridin-4-yl)pyrrolidin-2-one (143-2). To a solution of 4-bromo-2-nitropyridine (143-1) (230 mg, 1.13 mmol, 1.00 eq) and pyrrolidin-2-one (116 mg, 1.36 mmol, 104 μL, 1.20 eq) in dioxane (4 mL) were added Cs₂CO₃ (738 mg, 2.27 mmol, 2.00 eq), Xantphos (65.6 mg, 113 μmol, 0.100 eq) and Pd₂(dba)₃ (31.1 mg, 34.0 μmol, 0.0300 eq) at 25° C. under N₂ atmosphere. The mixture was stirred at 100° C. for 12 h. LCMS showed that the desired mass was detected. Water (3 mL) was added to the mixture at 25° C. Then the reaction mixture was extracted with EtOAc (2 mL*3). The organic layers were dried over anhydrous sodium sulfate, filtered and the filtrate was concentrated under reduced pressure to give a residue. The residue was purified by prep-TLC (SiO₂, Dichloromethane/Methanol=5/1) to give the title compound (143-2) (185 mg, 593 μmol, 52.3% yield, 66.4% purity) as a yellow solid. LCMS (ES⁺): m/z 208.1 [M+H]⁺.

Synthesis of 1-(2-aminopyridin-4-yl)pyrrolidin-2-one (143-3). To a solution of 1-(2-nitropyridin-4-yl)pyrrolidin-2-one (143-2) (175 mg, 845 μmol, 1.00 eq) in EtOAc (4 mL) was added 10% Pd/C (175 mg, 50% purity) at 25° C. under H₂ (15 Psi) atmosphere. The mixture was stirred at 25° C. for 20 min. TLC (SiO₂, Dichloromethane/Methanol=5/1) indicated 1-(2-nitropyridin-4-yl)pyrrolidin-2-one (143-2) was consumed completely and one new spot formed. The reaction mixture was filtered and the filtrate was concentrated under reduced pressure to give a residue to give the title compound (143-3) (130 mg, crude) as a yellow oil. LCMS (ES⁺): m/z 178.1 [M+H]⁺.

Synthesis of 1-(2-((5-chloro-4-(3-cyclohexylphenyl)pyrimidin-2-yl)amino)pyridin-4-yl)pyrrolidin-2-one (Compound 150). To a solution of 2,5-dichloro-4-(3-cyclohexylphenyl)pyrimidine (105 mg, 342 μmol, 1.00 eq) and 1-(2-aminopyridin-4-yl)pyrrolidin-2-one (143-3) (129 mg, 728 μmol, 2.13 eq) in dioxane (3 mL) were added Xantphos (59.3 mg, 103 μmol, 0.30 eq), Pd(OAc)₂ (38.4 mg, 171 μmol, 0.50 eq) and Cs₂CO₃ (223 mg, 684 μmol, 2.00 eq) at 25° C. under N₂ atmosphere. The mixture was stirred at 100° C. for 12 h. LCMS showed that the desired mass was detected. The reaction mixture was concentrated under reduced pressure to remove solvent. The residue were purified by prep-HPLC (column: Phenomenex Luna 80*30 mm*3 um; mobile phase: [H₂O (0.1% TFA)-ACN]; gradient: 35%-65% B over 8.0 min) to give the title compound (Compound 150) (5.10 mg, 10.5 μmol, 3.06% yield, 91.8% purity) as a yellow solid. LCMS (ES⁺): m/z 448.1 [M+H]⁺.

### Example 144. Synthesis of 1-(5-((4-(1-acetylpiperidin-3-yl)-5-chloropyrimidin-2-yl)amino)pyridin-3-yl)pyrrolidine-2-one (Compound 151)

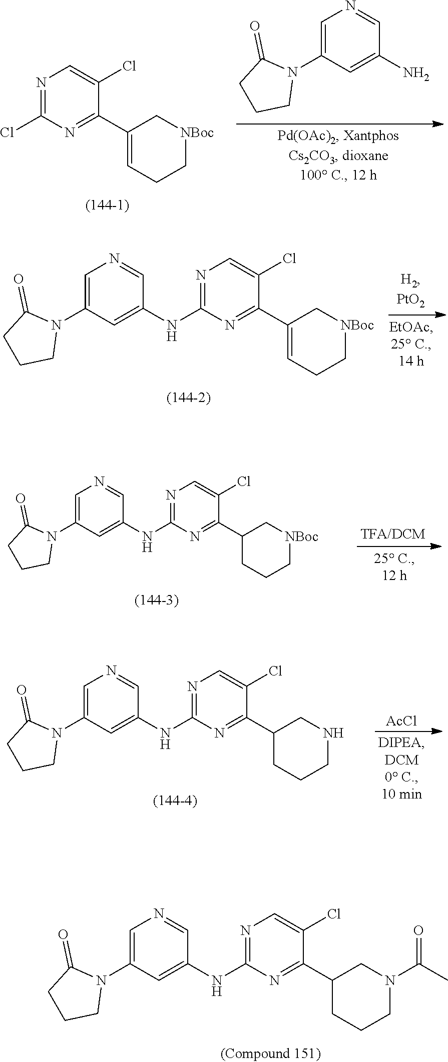

Synthesis of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (144-2). To a solution of 1-(5-amino-3-pyridyl)pyrrolidine-2-one (1.81 g, 8.18 mmol, 1.00 eq) and tert-butyl 5-(2,5-dichloropyrimidin-4-yl)-3,6-dihydropyridine-1(2H)-carboxylate (144-1) (2.70 g, 8.18 mmol, 1.00 eq) in dioxane (30 mL) were added Cs₂CO₃ (5.33 g, 16.4 mmol, 2.00 eq), Pd(OAc)₂ (91.8 mg, 409 μmol, 0.0500 eq) and Xantphos (142 mg, 245 μmol, 0.0300 eq) at 25° C. under N₂ atmosphere. The mixture was stirred at 100° C. for 12 h. LCMS showed that the desired mass was detected. Water (10 mL) was added to the mixture at 25° C. Then the reaction mixture was extracted with EtOAc (9 mL*3). The organic layers were dried over anhydrous sodium sulfate, filtered and the filtrate was concentrated under reduced pressure to give a residue. The residue was purified by prep-TLC (SiO₂, Dichloromethane/Methanol=20/1) to give the title compound (144-2) (1.50 g, 2.36 mmol, 28.9% yield, 74.2% purity) as a white solid. LCMS (ES⁺): m/z 471.3 [M+H]⁺.

Synthesis of tert-butyl 3-(5-chloro-2-((5-(2-oxopyrrolidin-1-yl)pyridin-3-yl)amino)pyrimidin-4-yl)piperidine-1-carboxylate (144-3). To a solution of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (144-2) (300 mg, 637 μmol, 1.00 eq) in EtOAc (5 mL) was added PtO2 (289 mg, 1.27 mmol, 2.00 eq) at 25° C. The mixture was stirred at 25° C. for 2 h under H₂ (15 psi). LCMS showed tert-butyl 5-(5-chloro-2-((5-(2-oxopyrrolidin-1-yl)pyridin-3-yl)amino)pyrimidin-4-yl)-3,6-dihydropyridine-1(2H)-carboxylate (144-2) was not consumed completely and the desired mass was detected. The mixture was added PtO2 (289 mg, 1.27 mmol, 2.00 eq) at 25° C. The mixture was stirred at 25° C. for 12 h under H₂ (15 psi). LCMS showed tert-butyl 5-(5-chloro-2-((5-(2-oxopyrrolidin-1-yl)pyridin-3-yl)amino)pyrimidin-4-yl)-3,6-dihydropyridine-1(2H)-carboxylate (144-2) was not consumed completely and the desired mass was detected. The reaction mixture was filtered and the filtrate was concentrated under reduced pressure to give a residue. The residue was purified by prep-TLC (SiO₂, Dichloromethane/Methanol=20/1) to give the title compound (144-3) (50.0 mg, 69.4 μmol, 10.9% yield, 65.6% purity) as a yellow oil. LCMS (ES⁺): m/z 473.2 [M+H]⁺.

Synthesis of 1-(5-((5-chloro-4-(piperidin-3-yl)pyrimidin-2-yl)amino)pyridin-3-yl)pyrrolidine-2-one (144-4). To a solution of tert-butyl 3-(5-chloro-2-((5-(2-oxopyrrolidin-1-yl)pyridin-3-yl)amino)pyrimidin-4-yl)piperidine-1-carboxylate (144-3) (50.0 mg, 106 μmol, 1.00 eq) in DCM (3 mL) was added TFA (0.5 mL) at 25° C. The mixture was stirred at 25° C. for 12 h. LCMS showed that the desired mass was detected. The reaction mixture was concentrated under reduced pressure to remove solvent to give the title compound (144-4) (50.0 mg, crude, TFA) as a colorless oil. LCMS (ES⁺): m/z 373.1 [M+H]⁺.

Synthesis of 1-(5-((4-(1-acetylpiperidin-3-yl)-5-chloropyrimidin-2-yl)amino)pyridin-3-yl)pyrrolidin-2-one (Compound 151). To a solution of 1-(5-((5-chloro-4-(piperidin-3-yl)pyrimidin-2-yl)amino)pyridin-3-yl)pyrrolidin-2-one (144-4) (39.4 mg, 106 μmol, 1.00 eq, TFA) in DCM (3 mL) were added DIPEA (13.7 mg, 106 μmol, 18.4 μL, 1.00 eq) and acetyl chloride (8.30 mg, 106 μmol, 7.52 μL, 1.00 eq) at 25° C. The mixture was stirred at 25° C. for 5 min. LCMS showed 1-(5-((5-chloro-4-(piperidin-3-yl)pyrimidin-2-yl)amino)pyridin-3-yl)pyrrolidin-2-one (144-4) was not consumed completely and the desired mass was detected. The mixture was stirred at 25° C. for 5 min. LCMS showed that the desired mass was detected. The reaction mixture was concentrated under reduced pressure to remove solvent. The residue were purified by prep-HPLC (column: Phenomenex Luna C18 75*30 mm*3 um; mobile phase: [H₂O (0.2% FA)-ACN]; gradient: 1%-40% B over 8.0 min) to give the title compound (Compound 151) (5.30 mg, 11.8 μmol, 11.1% yield, 92.0% purity) as a colorless oil. LCMS (ES⁺): m/z 415.0 [M+H]⁺.

### Example 145. Synthesis of 1-(5-((4-(1-acetylpiperidin-4-yl)-5-chloropyrimidin-2-yl)amino)pyridin-3-yl)pyrrolidin-2-one (Compound 152)

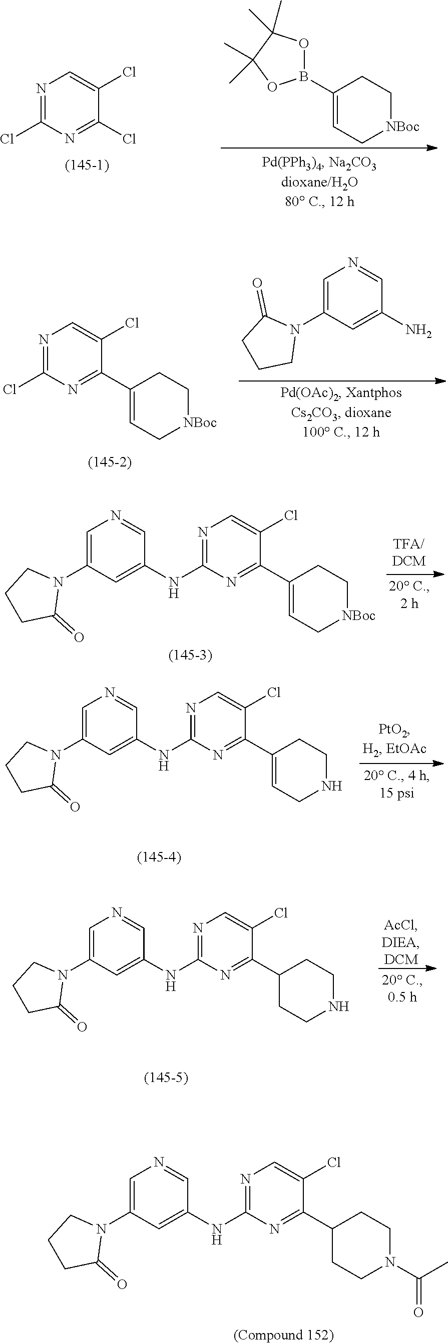

tert-butyl 4-(2,5-dichloropyrimidin-4-yl)-3,6-dihydropyridine-1(2H)-carboxylate (145-2). Two batches in parallel were set up. To a solution of tert-butyl 4-(4,4,5,5-tetramethyl-1,3,2-dioxaborolan-2-yl)-3,6-dihydro-2H-pyridine-1-carboxylate (2.60 g, 8.41 mmol, 0.800 eq) in dioxane (30 mL) and H₂O (8 mL) were added 2,4,5-trichloropyrimidine (145-1) (1.93 g, 10.5 mmol, 1.00 eq), Na₂CO₃ (2.23 g, 21.0 mmol, 2.00 eq) and Pd(PPh₃)₄ (243 mg, 210 μmol, 0.0200 eq) at 20° C. The mixture was stirred at 80° C. for 12 h under N₂ atmosphere. One batch in parallel was monitored in random. LCMS showed 2,4,5-trichloropyrimidine (145-1) was consumed and the desired compound was detected. Two batches in parallel were combined. H₂O (40 mL) was added to the solution, then the mixture was extracted with EtOAc (20 mL*3), the combined organic layers were dried over Na₂SO₄, then concentrated in vacuo. The mixture was purified by MPLC (SiO₂, Petroleum ether/Ethyl acetate=1/0 to 1/1) to give the title compound (145-2) (3.20 g, 9.69 mmol, 46.1% yield) as a black solid. LCMS (ES⁺): m/z 274.1 [M+H−56]⁺.

tert-butyl 4-(5-chloro-2-((5-(2-oxopyrrolidin-1-yl)pyridin-3-yl)amino)pyrimidin-4-yl)-3,6-dihydropyridine-1(2H)-carboxylate (145-3). To a solution of tert-butyl 4-(2,5-dichloropyrimidin-4-yl)-3,6-dihydro-2H-pyridine-1-carboxylate (145-2) (1.00 g, 2.42 mmol, 1.00 eq) and 1-(5-amino-3-pyridyl)pyrrolidin-2-one (537 mg, 2.42 mmol, 1.00 eq) in dioxane (10 mL) were added Xantphos (56.1 mg, 97.0 μmol, 0.0400 eq), Pd(OAc)₂ (32.6 mg, 145 μmol, 0.0600 eq) and Cs₂CO₃ (1.58 g, 4.85 mmol, 2.00 eq) at 20° C. The mixture was stirred at 100° C. for 12 h under N₂ atmosphere. LCMS showed tert-butyl 4-(2,5-dichloropyrimidin-4-yl)-3,6-dihydro-2H-pyridine-1-carboxylate (145-2) was consumed and the desired compound was detected. H₂O (10 mL) was added to the solution, then the mixture was extracted with EtOAc (10 mL*3), the combined organic lays were dried over Na₂SO₄, then concentrated in vacuo to give the crude product. The mixture was purified by MPLC (SiO₂, Petroleum ether/Ethyl acetate=1/0 to 1/1) to give the title compound (145-3) (1.00 g, 14.9 mmol, 87.6% yield) as a yellow solid. LCMS (ES⁺): m/z 471.2 [M+H]⁺.

1-(5-((5-chloro-4-(1,2,3,6-tetrahydropyridin-4-yl)pyrimidin-2-yl)amino)pyridin-3-yl)pyrrolidin-2-one (145-4). To a solution tert-butyl 4-[5-chloro-2-[[5-(2-oxopyrrolidin-1-yl)-3-pyridyl]amino]pyrimidin-4-yl]-3,6-dihydro-2H-pyridine-1-carboxylate (145-3) (200 mg, 424 μmol, 1.00 eq) in DCM (2 mL) was added TFA (0.4 mL) at 20° C. The mixture was stirred at 20° C. for 2 h. LCMS showed that tert-butyl 4-[5-chloro-2-[[5-(2-oxopyrrolidin-1-yl)-3-pyridyl]amino]pyrimidin-4-yl]-3,6-dihydro-2H-pyridine-1-carboxylate (145-3) was consumed and the desired mass was detected. The mixture was concentrated in vacuo to give the title compound (145-4) (200 mg, crude, TFA) as a yellow solid. LCMS (ES⁺): m/z 371.0 [M+H]⁺.

1-(5-((5-chloro-4-(piperidin-4-yl)pyrimidin-2-yl)amino)pyridin-3-yl)pyrrolidin-2-one (145-5). To a solution of 1-[5-[[5-chloro-4-(1,2,3,6-tetrahydropyridin-4-yl)pyrimidin-2-yl]amino]-3-pyridyl]pyrrolidin-2-one (145-4) (100 mg, 206 μmol, 1.00 eq, TFA) in EtOAc (2 mL) were added PtO₂ (50 mg) at 20° C. The mixture was stirred at 20° C. for 2 h under H₂ (15 Psi). LCMS showed that 1-[5-[[5-chloro-4-(1,2,3,6-tetrahydropyridin-4-yl)pyrimidin-2-yl]amino]-3-pyridyl]pyrrolidin-2-one (145-4) was consumed and the desired mass was detected. Then added PtO₂ (20.0 mg) at 20° C. The mixture was stirred at 20° C. for 2 h under H₂ (15 Psi). LC-MS showed that 1-[5-[[5-chloro-4-(1,2,3,6-tetrahydropyridin-4-yl)pyrimidin-2-yl]amino]-3-pyridyl]pyrrolidin-2-one (145-4) was consumed and the desired mass was detected. The mixture was filtered, the filtrate was concentrated in vacuo to give the title compound (145-5) (75.0 mg, 201 μmol, 97.5% yield) as a yellow solid. LCMS (ES⁺): m/z 373.1 [M+H]⁺.

1-(5-((4-(1-acetylpiperidin-4-yl)-5-chloropyrimidin-2-yl)amino)pyridin-3-yl)pyrrolidin-2-one (Compound 152). To a solution of 1-[5-[[5-chloro-4-(4-piperidyl)pyrimidin-2-yl]amino]-3-pyridyl]pyrrolidin-2-one (145-5) (75.0 mg, 154 μmol, 1.00 eq, TFA) in DCM (3 mL) was added DIEA (59.7 mg, 462 μmol, 80.5 μL, 3.00 eq) at 20° C., and added acetyl chloride (36.3 mg, 462 μmol, 32.9 μL, 3.00 eq) at 0° C. The mixture was stirred at 20° C. for 0.5 h. LCMS showed that the desired mass was detected. H₂O (3 mL) was added to the solution, then the mixture was extracted with EtOAc (3 mL*3), the combined organic layers were dried over Na₂SO₄, then concentrated in vacuo. The residue was purified by prep-HPLC (column: Phenomenex Luna C18 75*30 mm*3 um; mobile phase: [water (FA)-ACN]; B %: 5%-45%, 8 min) to give the title compound (Compound 152) (3.50 mg, 7.64 μmol, 4.96% yield, 90.6% purity) as a yellow solid. LCMS (ES⁺): m/z 415.1 [M+H]⁺.

### Example 146. Synthesis of 1-(5-((4-(1-cyclopropylpiperidin-3-yl)-5-fluoropyrimidin-2-yl)amino)pyridin-3-yl)pyrrolidine-2-one (Compound 154)

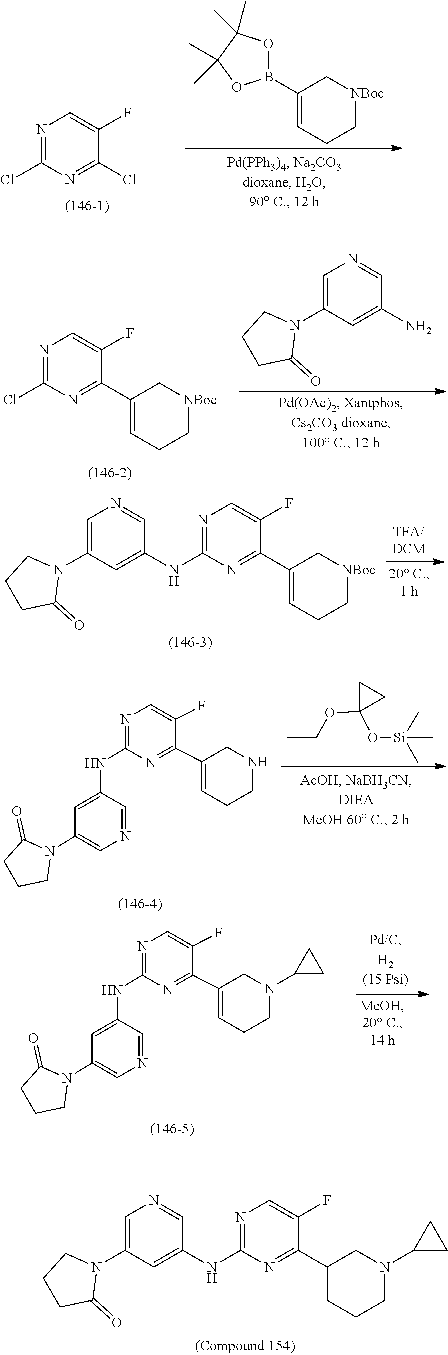

Synthesis of tert-butyl 5-(2-chloro-5-fluoropyrimidin-4-yl)-3,6-dihydropyridine-1(2H)-carboxylate (146-2). To a solution of 2,4-dichloro-5-fluoropyrimidine (146-1) (1.30 g, 7.76 mmol, 1.20 eq) in dioxane (30 mL) and H₂O (3 mL) were added Pd(PPh₃)₄ (149 mg, 129 μmol, 0.0200 eq), tert-butyl 5-(4,4,5,5-tetramethyl-1,3,2-dioxaborolan-2-yl)-3,6-dihydro-2H-pyridine-1-carboxylate (2.00 g, 6.47 mmol, 1.00 eq) and Na₂CO₃ (1.37 g, 12.9 mmol, 2.00 eq) at 20° C. under N₂ atmosphere. The mixture was stirred at 90° C. for 12 h. LCMS showed 2,4-dichloro-5-fluoropyrimidine (146-1) was consumed completely and the desired mass was detected. Water (25 mL) was added to the mixture at 20° C. Then the reaction mixture was extracted with EtOAc (20 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=1/0 to 10/1) to give the title compound (146-2) (1.80 g, 5.74 mmol, 88.7% yield) as a colorless oil. LCMS (ES⁺): m/z 257.9 [M+H−56]⁺

Synthesis of tert-butyl 5-(5-fluoro-2-((5-(2-oxopyrrolidin-1-yl)pyridin-3-yl)amino)pyrimidin-4-yl)-3,6-dihydropyridine-1(2H)-carboxylate (146-3). To a solution of tert-butyl 5-(2-chloro-5-fluoropyrimidin-4-yl)-3,6-dihydropyridine-1(2H)-carboxylate (146-2) (1.00 g, 3.19 mmol, 1.00 eq) in dioxane (20 mL) were added Pd(OAc)₂ (35.8 mg, 159 μmol, 0.0500 eq), Cs₂CO₃ (2.08 g, 6.37 mmol, 2.00 eq), 1-(5-amino-3-pyridyl)pyrrolidine-2-one (565 mg, 3.19 mmol, 1.00 eq) and Xantphos (55.3 mg, 95.6 μmol, 0.0300 eq) at 20° C. under N₂ atmosphere. The mixture was stirred at 100° C. for 12 h. LCMS showed tert-butyl 5-(2-chloro-5-fluoropyrimidin-4-yl)-3,6-dihydropyridine-1(2H)-carboxylate (146-2) was consumed completely and the desired mass was detected. Water (16 mL) was added to the mixture at 20° C. Then the reaction mixture was extracted with EtOAc (15 mL*3). The organic layers were dried over anhydrous sodium sulfate, filtered and the filtrate was concentrated under reduced pressure to give a residue. The residue was purified by column chromatography (SiO₂, Petroleum ether/Ethyl acetate=1/3 to 0/1) to give the title compound (146-3) (750 mg, 1.65 mmol, 51.8% yield) as a yellow oil. LCMS (ES⁺): m/z 455.1 [M+H]⁺.

Synthesis of 1-(5-((5-fluoro-4-(1,2,5,6-tetrahydropyridin-3-yl)pyrimidin-2-yl)amino)pyridin-3-yl)pyrrolidine-2-one (146-4). To a solution of tert-butyl 5-(5-fluoro-2-((5-(2-oxopyrrolidin-1-yl)pyridin-3-yl)amino)pyrimidin-4-yl)-3,6-dihydropyridine-1(2H)-carboxylate (146-3) (600 mg, 1.32 mmol, 1.00 eq) in DCM (6 mL) was added TFA (3.07 g, 26.9 mmol, 2 mL, 20.4 eq) at 20° C. The mixture was stirred at 20° C. for 1 h. LCMS showed tert-butyl 5-(5-fluoro-2-((5-(2-oxopyrrolidin-1-yl)pyridin-3-yl)amino)pyrimidin-4-yl)-3,6-dihydropyridine-1(2H)-carboxylate (146-3) was consumed completely and the desired mass was detected. The reaction mixture was concentrated under reduced pressure to remove solvent to give the title compound (146-4) (600 mg, crude, TFA) as a yellow oil. LCMS (ES⁺): m/z 355.1 [M+H]⁺.

Synthesis of 1-(5-((4-(I-cyclopropyl-1,2,5,6-tetrahydropyridin-3-yl)-5-fluoropyrimidin-2-yl)amino)pyridin-3-yl)pyrrolidin-2-one (146-5). To a solution of 1-(5-((5-fluoro-4-(1,2,5,6-tetrahydropyridin-3-yl)pyrimidin-2-yl)amino)pyridin-3-yl)pyrrolidin-2-one (146-4) (600 mg, 1.28 mmol, 1.00 eq, TFA) in MeOH (8 mL) was added DIEA (166 mg, 1.28 mmol, 223 μL, 1.00 eq) at 20° C. The mixture was stirred at 20° C. for 0.5 h. CH₃COOH (76.9 mg, 1.28 mmol, 73.3 μL, 1.00 eq) and (1-ethoxycyclopropoxy)-trimethyl-silane (971 mg, 5.57 mmol, 1.12 mL, 4.35 eq) were added to the mixture at 20° C. The mixture was stirred at 60° C. for 0.5 h. NaBH₃CN (241 mg, 3.84 mmol, 3.00 eq) was added to the mixture at 60° C. under N₂ atmosphere. The mixture was stirred at 60° C. for 30 min. LCMS showed ˜30.1% of 1-(5-((5-fluoro-4-(1,2,5,6-tetrahydropyridin-3-yl)pyrimidin-2-yl)amino)pyridin-3-yl)pyrrolidin-2-one (146-4) was remaining and desired mass was detected. Then the mixture was stirred at 60° C. for 0.5 h. LCMS showed 1-(5-((5-fluoro-4-(1,2,5,6-tetrahydropyridin-3-yl)pyrimidin-2-yl)amino)pyridin-3-yl)pyrrolidin-2-one (146-4) was consumed completely and the desired mass was detected. The reaction mixture was concentrated under reduced pressure to remove solvent. The residue was purified by column chromatography (SiO₂, Petroleum ether:Ethyl acetate=1:1 to 0:1) to give the title compound (146-5) (500 mg, 1.27 mmol, 99.0% yield) as a colorless oil. LCMS (ES⁺): m/z 395.1 [M+H]⁺.

Synthesis of 1-(5-((4-(1-cyclopropylpiperidin-3-yl)-5-fluoropyrimidin-2-yl)amino)pyridin-3-yl)pyrrolidin-2-one (Compound 154). To a solution of 1-(5-((4-(1-cyclopropyl-1,2,5,6-tetrahydropyridin-3-yl)-5-fluoropyrimidin-2-yl)amino)pyridin-3-yl)pyrrolidin-2-one (146-5) (450 mg, 1.14 mmol, 1.00 eq) in MeOH (5 mL) was added 10% Pd/C (450 mg, 50% purity) at 20° C. under H₂ (15 Psi) atmosphere. The mixture was stirred at 20° C. for 2 h. LCMS showed ˜75.0% of 1-(5-((4-(1-cyclopropyl-1,2,5,6-tetrahydropyridin-3-yl)-5-fluoropyrimidin-2-yl)amino)pyridin-3-yl)pyrrolidin-2-one (146-5) was remaining and desired mass was detected. Then the mixture was stirred at 20° C. under H₂ (15 Psi) atmosphere for 12 h. LCMS showed 1-(5-((4-(1-cyclopropyl-1,2,5,6-tetrahydropyridin-3-yl)-5-fluoropyrimidin-2-yl)amino)pyridin-3-yl)pyrrolidin-2-one (146-5) was consumed completely and the desired mass was detected. The reaction mixture was concentrated under reduced pressure to remove solvent. The residue was purified by prep-HPLC (column: Phenomenex Luna C18 200*40 mm*10 um; mobile phase: [H₂O (0.2% FA)-ACN]; gradient: 1%-25% B over 8.0 min) to give the title compound (Compound 154) (19.4 mg, 43.3 μmol, 3.79% yield, 98.7% purity, FA) as a white solid. LCMS (ES⁺): m/z 397.1 [M+H]⁺.

### Example 147 3-(5-((7-(2-(5-((4-([1,1′-biphenyl]-3-yl)-5-chloropyrimidin-2-yl)amino)pyridin-3-yl)-3-oxo-2,8-diazaspiro[4.5]decan-8-yl)-7-oxoheptyl)oxy)-1-oxoisoindolin-2-yl)piperidine-2,6-dione (Compound 166)

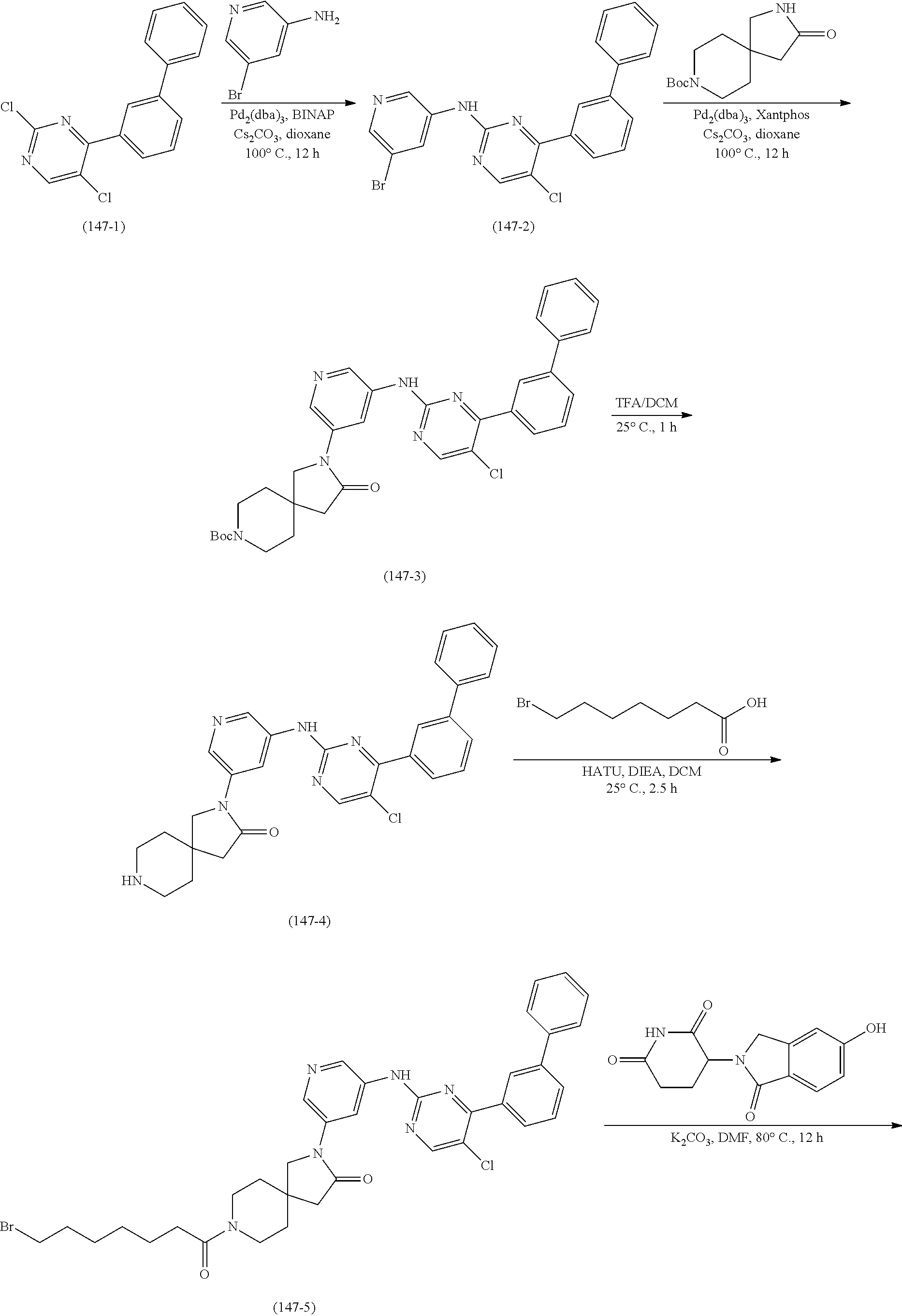

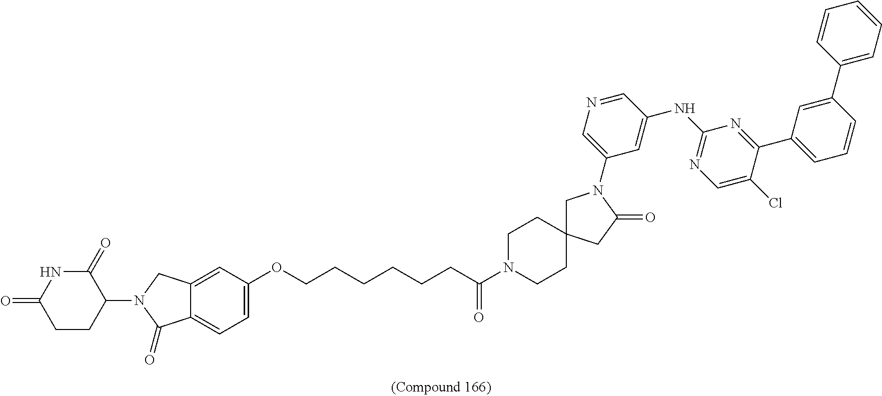 [SMILES: O=C1CCC(N2Cc3cc(OCCCCCCC(=O)N4CCC5(CC4)CC(=O)N(c4cncc(Nc6ncc(Cl)c(-c7cccc(-c8ccccc8)c7)n6)c4)C5)ccc3C2=O)C(=O)N1]

Synthesis of 4-([1,1′-biphenyl]-3-yl)-N-(5-bromopyridin-3-yl)-5-chloropyrimidin-2-amine (147-2). To a solution of 4-([1, 1′-biphenyl]-3-yl)-2, 5-dichloropyrimidine (147-1) (2.00 g, 6.64 mmol, 1.00 eq) and 5-bromopyridin-3-amine (1.15 g, 6.64 mmol, 1.00 eq) in dioxane (30 mL) were added BINAP (289 mg, 465 umol, 0.0700 eq), Cs₂CO₃ (4.33 g, 13.3 mmol, 2.00 eq) and Pd₂(dba)₃ (304 mg, 332 umol, 0.0500 eq) at 25° C. under N₂ atmosphere. The mixture was stirred at 100° C. for 12 h. LCMS showed 4-([1,1′-biphenyl]-3-yl)-2, 5-dichloropyrimidine (147-1) was consumed completely and the desired mass was detected. The mixture was diluted with H₂O (30 mL) and then extracted with EtOAc (20 mL*3). The combined organic layers were dried over Na₂SO₄, filtered and the filtrate was concentrated in vacuo. The mixture was purified by MPLC (SiO₂, Petroleum ether/Ethyl acetate=1/0 to 3/1) to give the title compound (147-2) (800 mg, 1.83 mmol, 27.5% yield) as a white solid. LCMS (ES⁺): m/z 437.2 [M+H]⁺.

Synthesis of tert-butyl 2-(5-((4-([1,1′-biphenyl]-3-yl)-5-chloropyrimidin-2-yl) amino) pyridin-3-yl)-3-oxo-2,8-diaz aspire [4.5]decane-8-carboxylate (147-3). To a solution of 4-([l, 1′-biphenyl]-3-yl)-N-(5-bromopyridin-3-yl)-5-chloropyrimidin-2-amine (147-2) (800 mg, 1.83 mmol, 1.00 eq) in dioxane (10.0 mL) were added tert-butyl 3-oxo-2,8-diazaspiro[4.5]decane-8-carboxylate (465 mg, 1.83 mmol, 1.00 eq), Pd₂(dba)₃ (50.3 mg, 54.9 umol, 0.0300 eq), Cs₂CO₃ (716 mg, 2.20 mmol, 1.20 eq) and Xantphos (95.3 mg, 165 umol, 0.0900 eq) at 25° C. under N₂ atmosphere. The mixture was stirred at 100° C. for 12 h. LCMS showed 4-([1,1′-biphenyl]-3-yl)-N-(5-bromopyridin-3-yl)-5-chloropyrimidin-2-amine (147-2) was consumed completely and the desired mass was detected. The mixture was diluted with H₂O (10 mL) and then extracted with EtOAc (5 mL*3). The combined organic layers were dried over Na₂SO₄, filtered and the filtrate was concentrated in vacuo. The mixture was purified by MPLC (SiO₂, Petroleum ether/Ethyl acetate=1/0 to 0/1) to give the title compound (147-3) (1.00 g, 1.64 mmol, 89.4% yield) as a white solid. LCMS (ES⁺): m/z 611.2 [M+H]⁺.

Synthesis of 2-(5-((4-([1,1′-biphenyl]-3-yl)-5-chloropyrimidin-2-yl) amino) pyridin-3-yl)-2,8-diazaspiro [4.5]decan-3-one (147-4). A solution of tert-butyl 2-(5-((4-([1,1′-biphenyl]-3-yl)-5-chloropyrimidin-2-yl) amino) pyridin-3-yl)-3-oxo-2,8-diaz aspire [4.5]decane-8-carboxylate (147-3) (500 mg, 818 umol, 1.00 eq) in DCM (6 mL) and TFA (2 mL) 25° C. The mixture was stirred at 25° C. for 1 h. LCMS showed tert-butyl 2-(5-((4-([1, 1′-biphenyl]-3-yl)-5-chloropyrimidin-2-yl) amino) pyridin-3-yl)-3-oxo-2,8-diaz aspire [4.5]decane-8-carboxylate (147-3) was consumed completely and the desired mass was detected. The mixture was concentrated in vacuo to give the title compound (147-4) (500 mg, crude, TFA) as a yellow oil. LCMS (ES⁺): m/z 511.2 [M+H]⁺.

Synthesis of 2-(5-((4-([1,1′-biphenyl]-3-yl)-5-chloropyrimidin-2-yl) amino) pyridin-3-yl)-8-(7-bromoheptanoyl)-2,8-diazaspiro [4.5]decan-3-one (147-5). To a stirred solution of 7-bromoheptanoic acid (115 mg, 549 μmol, 1.00 eq) and 2-(5-((4-([1, 1′-biphenyl]-3-yl)-5-chloropyrimidin-2-yl) amino) pyridin-3-yl)-2,8-diazaspiro [4.5]decan-3-one (147-4) (410 mg, 656 μmol, 1.20 eq, TFA) in DCM (8 mL) were added HATU (229 mg, 603 μmol, 1.10 eq), DIEA (213 mg, 1.65 mmol, 287 μL, 3.00 eq) at 25° C. The resulting mixture was stirred at 25° C. for 2 h. LCMS showed 7-bromoheptanoic acid (115 mg, 549 μmol, 1.00 eq) and 2-(5-((4-([1,1′-biphenyl]-3-yl)-5-chloropyrimidin-2-yl) amino) pyridin-3-yl)-2,8-diazaspiro [4.5]decan-3-one (147-4) was consumed completely and the desired mass was detected. The mixture was diluted with H₂O (5 mL) and then extracted with EtOAc (5 mL*3). The combined organic layers were dried over Na₂SO₄, filtered and the filtrate was concentrated in vacuo. The mixture was purified by MPLC (SiO₂, EtOAc/MeOH=1/0 to 10/1) to give the title compound (147-5) (380 mg, 541 μmol, 98.7% yield) as a yellow oil. LCMS (ES⁺): m/z 701.2 [M+H]⁺.

Synthesis of 3-(5-((7-(2-(5-((4-([1,1′-biphenyl]-3-yl)-5-chloropyrimidin-2-yl) amino) pyridin-3-yl)-3-oxo-2,8-diazaspiro[4.5]decan-8-yl)-7-oxoheptyl) oxy)-1-oxoisoindolin-2-yl) piperidine-2, 6-dione (Compound 166). To a solution of 2-(5-((4-([1,1′-biphenyl]-3-yl)-5-chloropyrimidin-2-yl) amino) pyridin-3-yl)-2,8-diazaspiro [4.5]decan-3-one (147-5) (300 mg, 427 umol, 1.00 eq), 3-(5-hydroxy-1-oxoisoindolin-2-yl)piperidine-2,6-dione (111 mg, 427 umol, 1.00 eq) in DMF (4.00 mL) was added K₂CO₃ (177 mg, 1.28 mmol, 3.00 eq) at 25° C. After addition, the mixture was stirred at 80° C. for 12 h. LCMS showed 2-(5-((4-([1, 1′-biphenyl]-3-yl)-5-chloropyrimidin-2-yl) amino) pyridin-3-yl)-2,8-diazaspiro [4.5]decan-3-one (147-5) was consumed completely and the desired mass was detected. H₂O (5 mL) was added to the solution, the mixture was extracted with EtOAc (5 ml*3), then the combined organic layers were washed with brine (10 mL*3), dried over Na₂SO₄, then concentrated in vacuo to give the crude product. The residue was purified by prep-HPLC (column: Waters Xbridge BEH C18 100*30 mm * 10 um; mobile phase: [water (NH₄HCO₃)-ACN]; B %: 40%) to give (Compound 166) (7.20 mg, 7.70 μmol, 1.80% yield, 94.3% purity). LCMS (ES⁺): m/z 881.2 [M+H]⁺.

### Example 148. N-(5-((4-([1,1′-biphenyl]-3-yl)-5-chloropyrimidin-2-yl)amino)pyridin-3-yl)-10-(2-((1-(2,6-dioxopiperidin-3-yl)-2-oxo-1,2-dihydrobenzo[cd]indol-6-yl)oxy)acetamido)decanamide (Compound 167)

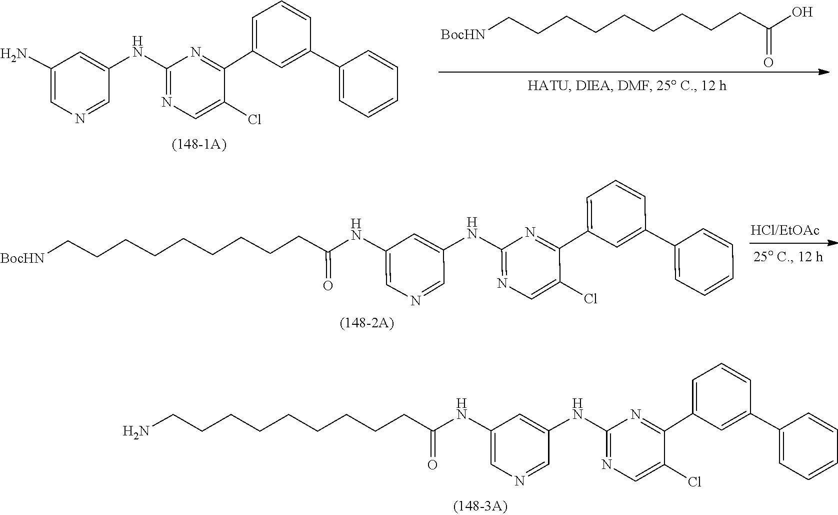 [SMILES: Nc1cncc(Nc2ncc(Cl)c(-c3cccc(-c4ccccc4)c3)n2)c1 ; CC(C)(C)OC(=O)NCCCCCCCCCC(=O)O ; CC(C)(C)OC(=O)NCCCCCCCCCC(=O)Nc1cncc(Nc2ncc(Cl)c(-c3cccc(-c4ccccc4)c3)n2)c1 ; NCCCCCCCCCC(=O)Nc1cncc(Nc2ncc(Cl)c(-c3cccc(-c4ccccc4)c3)n2)c1]

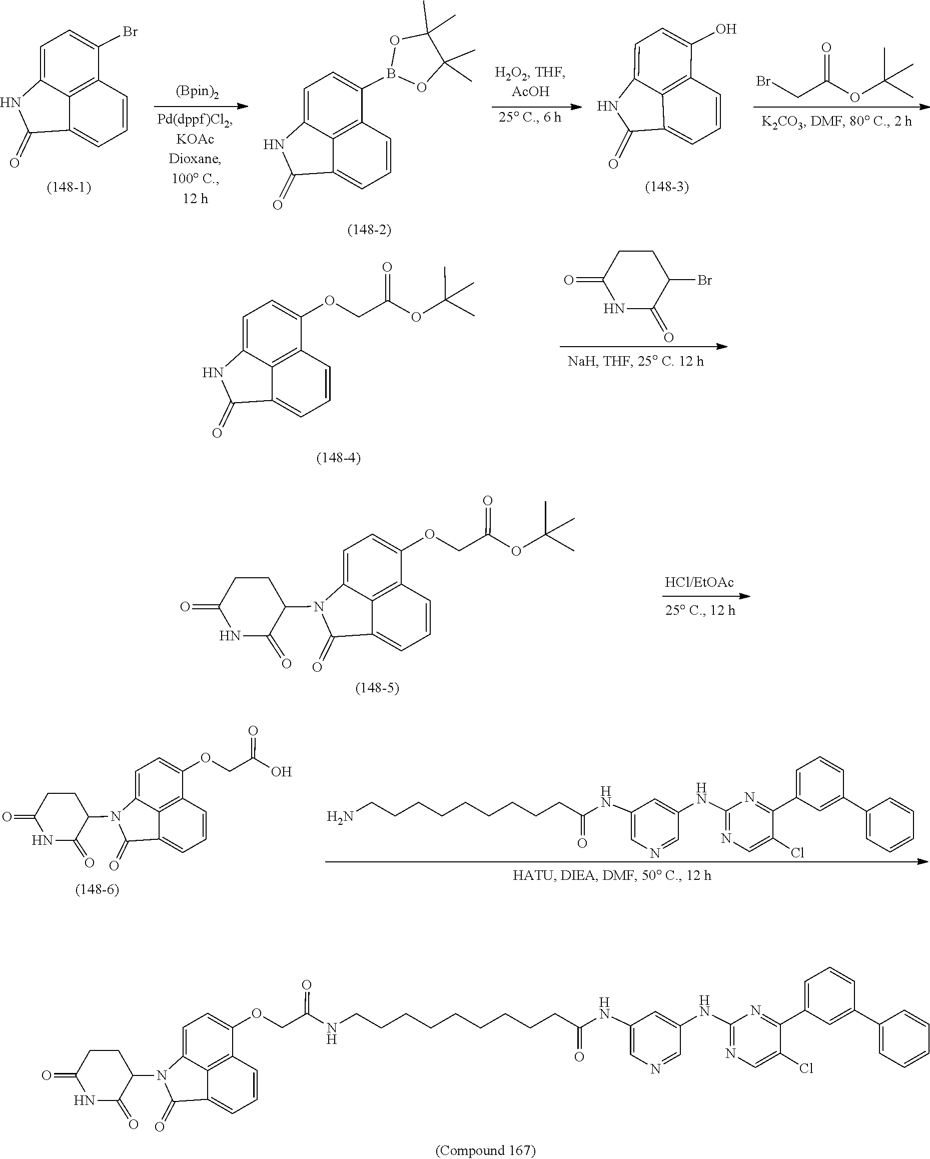

Synthesis of tert-butyl (10-((5-((4-([1,1′-biphenyl]-3-yl)-5-chloropyrimidin-2-yl)amino)pyridin-3-yl)amino)-10-oxodecyl)carbamate (148-2A). To a solution of 10-(tert-butoxycarbonylamino)decanoic acid (2.00 g, 6.96 mmol, 1.00 eq) and N³-[5-chloro-4-(3-phenylphenyl)pyrimidin-2-yl]pyridine-3,5-diamine (148-1A) (2.34 g, 6.26 mmol, 0.900 eq) in DMF (30 mL) were added DIEA (2.70 g, 20.9 mmol, 3.64 mL, 3.00 eq) and HATU (3.18 g, 8.35 mmol, 1.20 eq) at 25° C. The mixture was stirred at 25° C. for 12 h. LCMS showed that N3-(4-([1,1′-biphenyl]-3-yl)-5-chloropyrimidin-2-yl)pyridine-3,5-diamine (148-1A) was consumed completely and the desired mass was detected. The reaction mixture was diluted with H₂O 20 mL and extracted with ethyl acetate (10 mL*3). The combined organic layers were concentrated under reduced pressure. The residue was purified by column chromatography (SiO₂, Petroleum ether/Ethyl acetate=1/1 to 0/1) to give the title compound (148-2A) (1.00 g, 1.55 mmol, 22.3% yield) as a yellow solid. LCMS (ES⁺): m/z 643.3 [M+H]⁺.

Synthesis of N-(5-((4-([1,1′-biphenyl]-3-yl)-5-chloropyrimidin-2-yl)amino)pyridin-3-yl)-10-aminodecanamide (148-3A). A solution of tert-butyl (10-((5-((4-([1,1′-biphenyl]-3-yl)-5-chloropyrimidin-2-yl)amino)pyridin-3-yl)amino)-10-oxodecyl)carbamate (148-2A) (400 mg, 622 umol, 1.00 eq) in HCl/EtOAc (5 mL) was stirred at 25° C. for 12 h. LCMS showed that tert-butyl (10-((5-((4-([1,1′-biphenyl]-3-yl)-5-chloropyrimidin-2-yl)amino)pyridin-3-yl)amino)-10-oxodecyl)carbamate (148-2A) was consumed completely and the desired mass was detected. The reaction mixture was concentrated under reduced pressure to give the title compound (148-3A) (300 mg, crude) as a brown solid. LCMS (ES⁺): m/z 543.3 [M+H]⁺.

Synthesis of 6-(4,4,5,5-tetramethyl-1,3,2-dioxaborolan-2-yl)benzo[cd]indol-2(1H)-one (148-2). To a solution of 6-bromo-1H-benzo[cd]indol-2-one (148-1) (5.00 g, 20.2 mmol, 1.00 eq) and 4,4,5,5-tetramethyl-2-(4,4,5,5-tetramethyl-1,3,2-dioxaborolan-2-yl)-1,3,2-dioxaborolane (15.4 g, 60.5 mmol, 3.00 eq) in dioxane (50 mL) were added Pd(dppf)Cl₂·CH₂Cl₂ (1.65 g, 2.02 mmol, 0.100 eq) and KOAc (5.93 g, 60.5 mmol, 3.00 eq) at 20° C. The mixture was stirred at 100° C. for 12 h under N₂ atmosphere. LCMS showed that 6-bromo-1H-benzo[cd]indol-2-one (148-1) was remained and the desired mass was detected. The reaction mixture was partitioned between EtOAc (10 mL*3) and H₂O 10 mL. The organic phase was separated, washed with brine 10 mL, dried over Na₂SO₄, filtered and the filtrate was concentrated under reduced pressure to give the title compound (148-2) (4 g, crude) as a brown solid. LCMS (ES⁺): m/z 296.0 [M+H]⁺.

Synthesis of 6-hydroxybenzo[cd]indol-2(1H)-one (148-3). To a solution of 6-(4,4,5,5-tetramethyl-1,3,2-dioxaborolan-2-yl)-1H-benzo[cd]indol-2-one (1.00 g, 3.39 mmol, 1.00 eq) (148-2) in THE (20 mL) were added H₂O₂(1.69 g, 14.9 mmol, 1.43 mL, 30.0% purity, 4.40 eq) and AcOH (1.02 g, 16.9 mmol, 969 μL, 5.00 eq) at 25° C. The mixture was stirred at 25° C. for 6 h. LCMS showed that 6-(4,4,5,5-tetramethyl-1,3,2-dioxaborolan-2-yl)-1H-benzo[cd]indol-2-one (148-2) was remained and the desired mass was detected. The reaction mixture was partitioned between EtOAc (10 mL*3) and H₂O 10 mL. The organic phase was separated, washed with brine (10 mL), dried over Na₂SO₄, filtered and the filtrate was concentrated under reduced pressure to give the title compound (148-3) (400 mg, crude) as a brown solid. LCMS (ES⁺): m/z 185.9 [M+H]⁺.

Synthesis of tert-butyl 2-((2-oxo-1, 2-dihydrobenzo[cd]indol-6-yl)oxy)acetate (148-4). To a solution of 6-hydroxybenzo[cd]indol-2(1H)-one (110 mg, 594 umol, 1.00 eq) (148-3) and tert-butyl 2-bromoacetate (139 mg, 713 umol, 105 μL, 1.20 eq) in DMF (2 mL) was added K₂CO₃ (246 mg, 1.78 mmol, 3.00 eq) at 25° C. The mixture was stirred at 80° C. for 2 h. LCMS showed that 6-hydroxybenzo[cd]indol-2(1H)-one (148-3) remained and the desired mass was detected. The reaction mixture was concentrated under reduced pressure to give the title compound (148-4) (120 mg, 401 umol, 67.5% yield) as a brown solid. LCMS (ES⁺): m/z 300.1 [M+H]⁺.

Synthesis of tert-butyl 2-((1-(2, 6-dioxopiperidin-3-yl)-2-oxo-1,2-dihydrobenzo[cd]indol-6-yl)oxy)acetate (148-5). To a solution of tert-butyl 2-[(2-oxo-1H-benzo[cd]indol-6-yl)oxy]acetate (120 mg, 401 umol, 1.00 eq) (59-4) and 3-bromopiperidine-2,6-dione (385 mg, 2.00 mmol, 5.00 eq) in THF (2.5 mL) was added NaH (160 mg, 4.01 mmol, 60.0% purity, 10.0 eq) at 0° C. The mixture was stirred at 25° C. for 12 h. LCMS showed that tert-butyl 2-[(2-oxo-1H-benzo[cd]indol-6-yl)oxy]acetate (59-4) remained and the desired mass was detected. The reaction mixture was partitioned between EtOAc (10 mL*3) and H₂O 10 mL. The organic phase was separated, washed with brine (10 mL), dried over Na₂SO₄, filtered and the filtrate was concentrated under reduced pressure to give the title compound (148-5) (150 mg, crude) as a brown solid. LCMS (ES⁺): m/z 411.1 [M+H]⁺.

Synthesis of 2-((1-(2, 6-dioxopiperidin-3-yl)-2-oxo-1,2-dihydrobenzo[cd]indol-6-yl)oxy)acetic acid (148-6). A solution of tert-butyl 2-[1-[(3R)-2,6-dioxo-3-piperidyl]-2-oxo-benzo[cd]indol-6-yl]oxyacetate (50.0 mg, 122 umol, 1.00 eq) (148-5) in HCl/EtOAc (4 M, 1 mL) was stirred at 25° C. for 12 h. LCMS showed that tert-butyl 2-[1-[(3R)-2,6-dioxo-3-piperidyl]-2-oxo-benzo[cd]indol-6-yl]oxyacetate (148-5) remained a little and the desired mass was detected. The reaction mixture was concentrated under reduced pressure to give the title compound (148-6) (40.0 mg, 113 umol, 92.7% yield) as a brown solid. LCMS (ES⁺): m/z 355.3 [M+H]⁺.

Synthesis of N-(5-((4-([1,1′-biphenyl]-3-yl)-5-chloropyrimidin-2-yl)amino)pyridin-3-yl)-10-(2-((1-(2,6-dioxopiperidin-3-yl)-2-oxo-1,2-dihydrobenzo[cd]indol-6-yl)oxy)acetamido)decanamide (Compound 167). To a solution of 2-((1-(2,6-dioxopiperidin-3-yl)-2-oxo-1,2-dihydrobenzo[cd]indol-6-yl)oxy)acetic acid (40.0 mg, 113 umol, 1.00 eq) (148-6) and 10-amino-N-[5-[[5-chloro-4-(3-phenylphenyl)pyrimidin-2-yl]amino]-3-pyridyl]decanamide (61.3 mg, 113 umol, 1.00 eq) in DMF (2 mL) were added HATU (42.9 mg, 113 umol, 1.00 eq) and DIEA (43.8 mg, 339 umol, 59.0 uL, 3.00 eq) at 25° C. The mixture was stirred at 50° C. for 12 h. LCMS showed that 2-((1-(2,6-dioxopiperidin-3-yl)-2-oxo-1,2-dihydrobenzo[cd]indol-6-yl)oxy)acetic acid (148-6) remained and the desired mass was detected. The reaction mixture was filtered and the filtrate was purified by prep-HPLC (column: C18-6 100*30 mm*5 um; mobile phase: [water (FA)-ACN]; B %: 45%-75%, 8 min) to give the title compound (Compound 167) (6.20 mg, 7.05 umol, 6.25% yield) as a brown solid. LCMS (ES⁺): m/z 879.4 [M+H]⁺.

### Example 149. Synthesis of 3-((4-(4-(2-(2-(5-((5-chloro-4-(piperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 168)

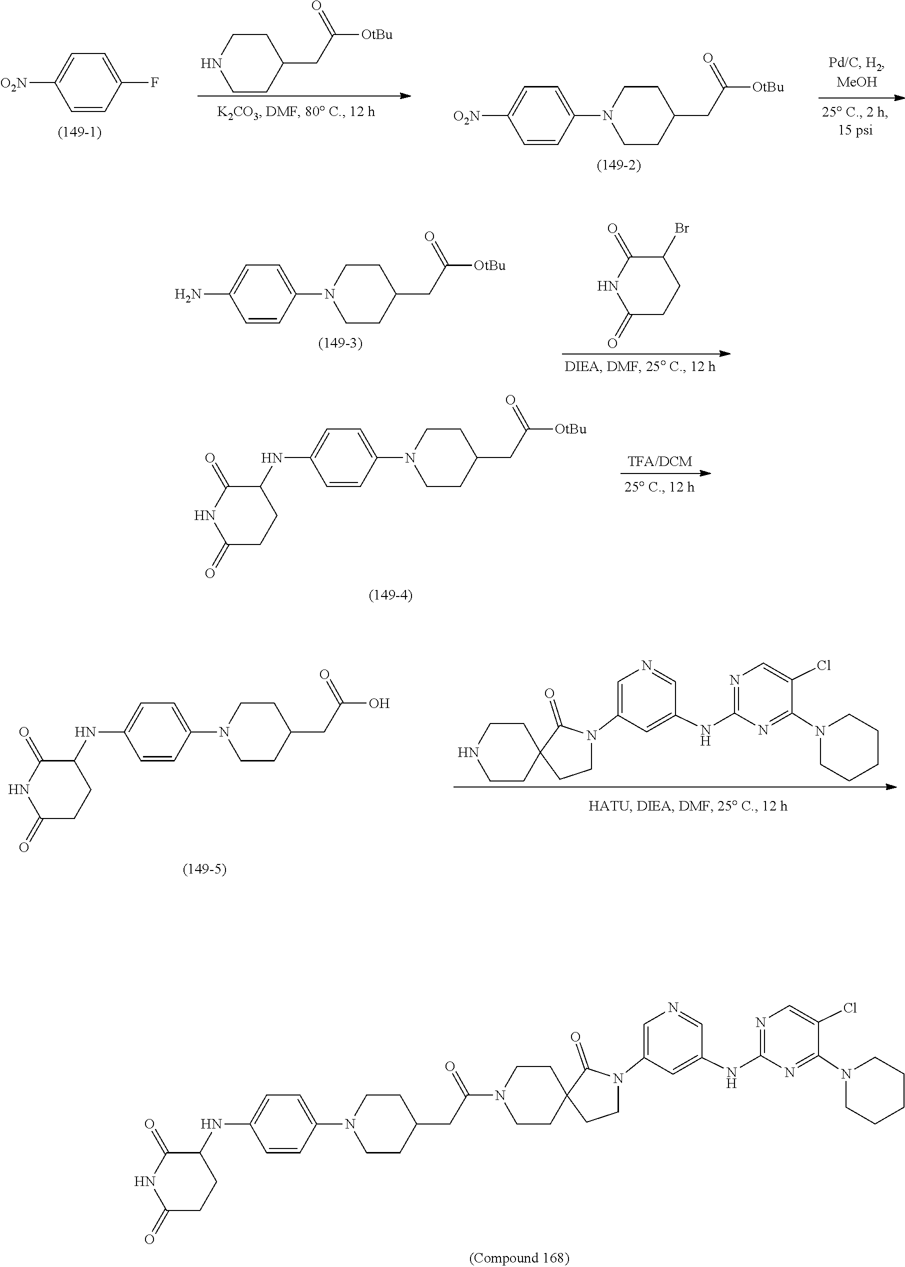

Synthesis of tert-butyl 2-(1-(4-nitrophenyl)piperidin-4-yl)acetate (149-2). To solution of 1-fluoro-4-nitrobenzene (149-1) (708 mg, 5.02 mmol, 532 μL, 1.00 eq) and tert-butyl 2-(piperidin-4-yl)acetate (1.00 g, 5.02 mmol, 1.00 eq) in DMF (10 mL) was added K₂CO₃ (832 mg, 6.02 mmol, 1.20 eq) at 25° C. The mixture was stirred at 80° C. for 12 h. LCMS showed that reaction was complete. The crude product was triturated with H₂O (20 mL*3), then the solid was dried under reduced pressure to give the title compound (149-2) (880 mg, crude) as a yellow solid. LCMS (ES⁺): m/z 321.2 [M+H]⁺.

Synthesis of tert-butyl 2-(1-(4-aminophenyl)piperidin-4-yl)acetate (149-3). To a mixture of tert-butyl 2-(1-(4-nitrophenyl)piperidin-4-yl)acetate (149-2) (780 mg, 2.43 mmol, 1.00 eq) in MeOH (10 mL) was added 10% Pd/C (780 mg, 50% purity) at 25° C. under N₂. The suspension was degassed under vacuum and purged with H₂ 4 times. The mixture was stirred under H₂ (15 psi) at 25° C. for 2 h. LCMS shows tert-butyl 2-(1-(4-nitrophenyl)piperidin-4-yl)acetate (149-2) was consumed and the desired mass was detected. The mixture was filtered and the filtrate was concentrated in vacuo to give the title compound (149-3) (582 mg, crude) was obtained as a brown solid. LCMS (ES⁺): m/z 291.3 [M+H]⁺.

Synthesis of tert-butyl 2-(1-(4-((2,6-dioxopiperidin-3-yl)amino)phenyl)piperidin-4-yl)acetate (149-4). To solution of tert-butyl 2-(1-(4-aminophenyl)piperidin-4-yl)acetate (149-3) (580 mg, 2.00 mmol, 1.00 eq) and 3-bromopiperidine-2,6-dione (959 mg, 4.99 mmol, 2.50 eq) in DMF (6 mL) was added DIEA (774 mg, 5.99 mmol, 1.04 mL, 3.00 eq). The mixture was stirred at 25° C. for 12 h. LCMS showed that reaction was complete. The residue was triturated with H₂O (35 mL), filtered and the filter cake was dried in vacuum to give the title compound (149-4) (613 mg, crude) as a brown solid. LCMS (ES⁺): m/z 402.2 [M+H]⁺.

Synthesis of 2-(1-(4-((2,6-dioxopiperidin-3-yl)amino)phenyl)piperidin-4-yl)acetic acid (149-5). To a solution of tert-butyl 2-(1-(4-((2,6-dioxopiperidin-3-yl)amino)phenyl)piperidin-4-yl)acetate (149-4) (610 mg, 1.52 mmol, 1.00 eq) in DCM (6 mL) was added TFA (2 mL) at 25° C. The mixture was stirred at 25° C. for 12 h. LCMS shows tert-butyl 2-(1-(4-((2,6-dioxopiperidin-3-yl)amino)phenyl)piperidin-4-yl)acetate (149-4) was consumed and the desired mass was detected. The reaction mixture was concentrated under reduced pressure to remove solvent to give the title compound (149-5) (530 mg, crude) as a brown oil. LCMS (ES⁺): m/z 346.1 [M+H]⁺.

Synthesis of 3-((4-(4-(2-(2-(5-((5-chloro-4-(piperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 168). To solution of 2-(1-(4-((2,6-dioxopiperidin-3-yl)amino)phenyl)piperidin-4-yl)acetic acid (149-5) (200 mg, 579 umol, 1.00 eq) and 2-(5-((5-chloro-4-(piperidin-1-yl)pyrimidin-2-yl)amino)pyridin-3-yl)-2,8-diazaspiro[4.5]decan-1-one (386 mg, 695 umol, 1.20 eq, TFA) in DMF (3 mL) were added HATU (330 mg, 869 umol, 1.50 eq), HOBt (39.1 mg, 290 umol, 0.500 eq), DIPEA (225 mg, 1.74 mmol, 303 μL, 3.00 eq) at 25° C. The mixture was stirred at 25° C. for 12 h. LCMS showed 2-(1-(4-((2,6-dioxopiperidin-3-yl)amino)phenyl)piperidin-4-yl)acetic acid (149-5) was consumed and the desired mass was detected. The mixture was concentrated in vacuo. The mixture was diluted with H₂O (5 mL) and then extracted with DCM (5 mL*3). The combined organic layers were dried over Na₂SO₄, filtered and the filtrate was concentrated in vacuo. The residue was purified by prep-HPLC (column: Waters Xbridge BEH C 18 100*30 mm*10 um; mobile phase: [water (NH₄HCO₃)-ACN]; B %: 35%-65%, 8 min) to give the title compound (Compound 168) (34.9 mg, 43.1 μmol, 9.90% yield, 95.0% purity) as a gray solid. LCMS (ES⁺): m/z 769.3 [M+H]⁺.

### Example 150. Synthesis of 3-((4-(1-(2-(2-(5-((5-chloro-4-((S)-3-hydroxypiperidin-1-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 169)

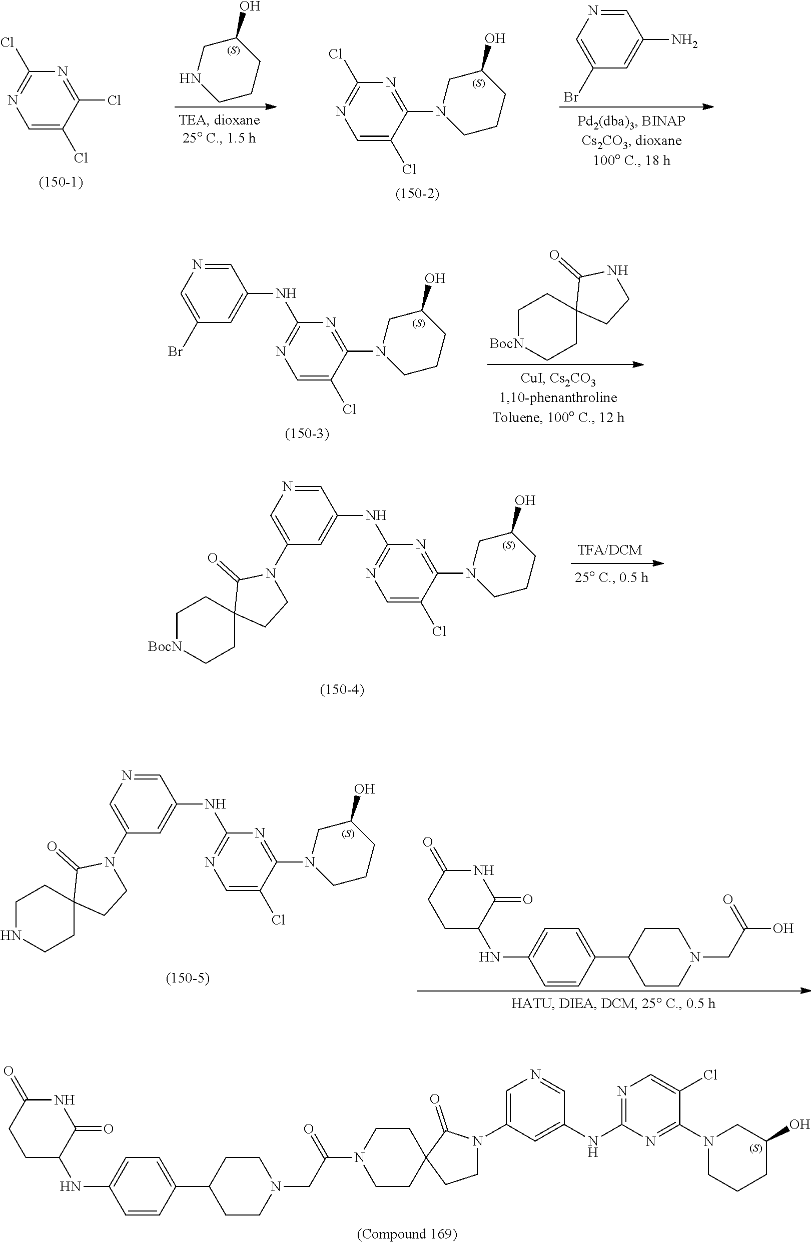

Synthesis of (S)-1-(2,5-dichloropyrimidin-4-yl)piperidin-3-ol (150-2). Two batches in parallel were set up. To a solution of 2,4,5-trichloropyrimidine (150-1) (816 mg, 4.45 mmol, 1.00 eq) and TEA (900 mg, 8.90 mmol, 1.24 mL, 2.00 eq) in dioxane (5 mL) was added (S)-piperidin-3-ol (450 mg, 4.45 mmol, 1.00 eq), the mixture was stirred at 25° C. for 1.5 h. LCMS showed 2,4,5-trichloropyrimidine (150-1) was consumed, desired target MS was detected. Two batches in parallel were combined. H₂O (10 mL) was added to the solution, the mixture was extracted with EtOAc (5 mL*3), then the combined organic layers were dried over Na₂SO₄, then concentrated in vacuo. The mixture was purified by MPLC (SiO₂, Petroleum ether/Ethyl acetate=1/0 to 1/1) to give title compound (150-2) (1.60 g, 6.45 mmol, 72.5% yield) as a yellow oil. LCMS (ES⁺): m/z 248.0 [M+H]⁺.

Synthesis of (S)-1-(2-((5-bromopyridin-3-yl)amino)-5-chloropyrimidin-4-yl)piperidin-3-ol (150-3). Two batches in parallel were set up. To a solution of (S)-1-(2,5-dichloropyrimidin-4-yl)piperidin-3-ol (150-2) (800 mg, 3.22 mmol, 1.00 eq) and 5-bromopyridin-3-amine (1.12 g, 6.45 mmol, 2.00 eq) in dioxane (10 mL) were added Cs₂CO₃ (2.10 g, 6.45 mmol, 2.00 eq), BINAP (201 mg, 322 umol, 0.100 eq) and Pd₂(dba)₃ (295 mg, 322 umol, 0.100 eq) at 25° C., the mixture was stirred at 100° C. for 6 h under N₂ atmosphere. LCMS showed (S)-1-(2,5-dichloropyrimidin-4-yl)piperidin-3-ol (150-2) was remained, desired target MS was detected. Then Cs₂CO₃ (735 mg, 2.26 mmol, 0.700 eq), BINAP (60.2 mg, 96.7 umol, 0.0300 eq) and Pd₂(dba)₃ (88.6 mg, 96.7 umol, 0.0300 eq) was added to the mixture, the mixture was stirred at 100° C. for 12 h under N₂ atmosphere, LCMS showed (S)-1-(2,5-dichloropyrimidin-4-yl)piperidin-3-ol (150-2) was consumed, desired target MS was detected. Two batches in parallel were combined. H₂O (10 mL) was added to the solution, the mixture was extracted with EtOAc (10 mL*3), then the combined organic layers were dried over Na₂SO₄, then concentrated in vacuo to give the crude product. The mixture was purified by MPLC (SiO₂, Petroleum ether/Ethyl acetate 1/0 to 1/0) to give the title compound (150-3) (724 mg, 1.88 mmol, 29.2% yield) as a yellow oil. LCMS (ES⁺): m/z 383.9 [M+H]⁺.

Synthesis of tert-butyl (S)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (150-4). To a solution of (S)-1-(2-((5-bromopyridin-3-yl)amino)-5-chloropyrimidin-4-yl)piperidin-3-ol (150-3) (195 mg, 507 μmol, 1.00 eq) and tert-butyl 1-oxo-2,8-diazaspiro[4.5]decane-8-carboxylate (155 mg, 608 umol, 1.20 eq) in Tol. (3 mL) was added Cs₂CO₃ (496 mg, 1.52 mmol, 3.00 eq), 1,10-PHENANTHROLINE (54.8 mg, 304 μmol, 0.600 eq) and CuI (29.0 mg, 152 umol, 0.300 eq) at 25° C., the mixture was stirred at 100° C. for 12 h under N₂ atmosphere. LCMS showed (S)-1-(2-((5-bromopyridin-3-yl)amino)-5-chloropyrimidin-4-yl)piperidin-3-ol (150-3) was consumed partly, desired target MS was detected. The mixture was concentrated to dryness under a stream of nitrogen, H₂O (5 mL) was added to the solution, the mixture was extracted with EtOAc (5 mL*3), then the combined organic lays were dried over Na₂SO₄, then concentrated in vacuo to give the crude product. The mixture was purified by prep-TLC (SiO₂, DCM/MeoH=10/1) to give the title compound (150-4) (95.0 mg, 170 umol, 33.6% yield) as a yellow solid. LCMS (ES⁺): m/z 558.2 [M+H]⁺.

Synthesis of (S)-2-(5-((5-chloro-4-(3-hydroxypiperidin-1-yl)pyrimidin-2-yl)amino)pyridin-3-yl)-2,8-diazaspiro[4.5]decan-1-one (150-5). A solution of tert-butyl (S)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (150-4) (95.0 mg, 170 μmol, 1.00 eq) in DCM (2 mL) and TFA (0.7 mL) was stirred at 25° C. for 0.5 h. LCMS showed tert-butyl (S)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (150-4) was consumed completely, desired target MS was detected. The mixture was concentrated in vacuo to give the title compound (150-5) (96.0 mg, crude, TFA) as a yellow oil. LCMS (ES⁺): m/z 458.2 [M+H]⁺.

Synthesis of (S)-2-(5-((5-chloro-4-(3-hydroxypiperidin-1-yl)pyrimidin-2-yl)amino)pyridin-3-yl)-2,8-diazaspiro[4.5]decan-1-one (Compound 169). To a solution of 2-(4-(4-((2,6-dioxopiperidin-3-yl)amino)phenyl)piperidin-1-yl)acetic acid (51.3 mg, 149 μmol, 1.00 eq) in DMF (1 mL) were added HATU (67.8 mg, 178 μmol, 1.20 eq), DIEA (96.0 mg, 743.0 μmol, 129 μL, 5.00 eq) at 25° C., then (S)-2-(5-((5-chloro-4-(3-hydroxypiperidin-1-yl)pyrimidin-2-yl)amino)pyridin-3-yl)-2,8-diazaspiro[4.5]decan-1-one (150-5) (85.0 mg, 149 μmol, 1.00 eq, TFA) was added to the mixture, the mixture was stirred at 25° C. for 0.5 h. LCMS showed (S)-2-(5-((5-chloro-4-(3-hydroxypiperidin-1-yl)pyrimidin-2-yl)amino)pyridin-3-yl)-2,8-diazaspiro[4.5]decan-1-one (150-5) was consumed, desired target MS was detected. The mixture was concentrated in vacuo. The residue was purified by prep-HPLC (column: Phenomenex Luna C18 200*40 mm*10 um; mobile phase: [water (FA)-ACN]; B %: 10%-45%, 8 min) to give the title compound (Compound 169) (15.2 mg, 17.9 μmol, 12.0% yield, 92.4% purity) as a white solid. LCMS (ES⁺): m/z 785.3 [M+H]⁺.

### Example 151. Synthesis of 3-((4-(1-(2-(2-(5-((5-chloro-4-((R)-3-hydroxypiperidin-1-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 170)

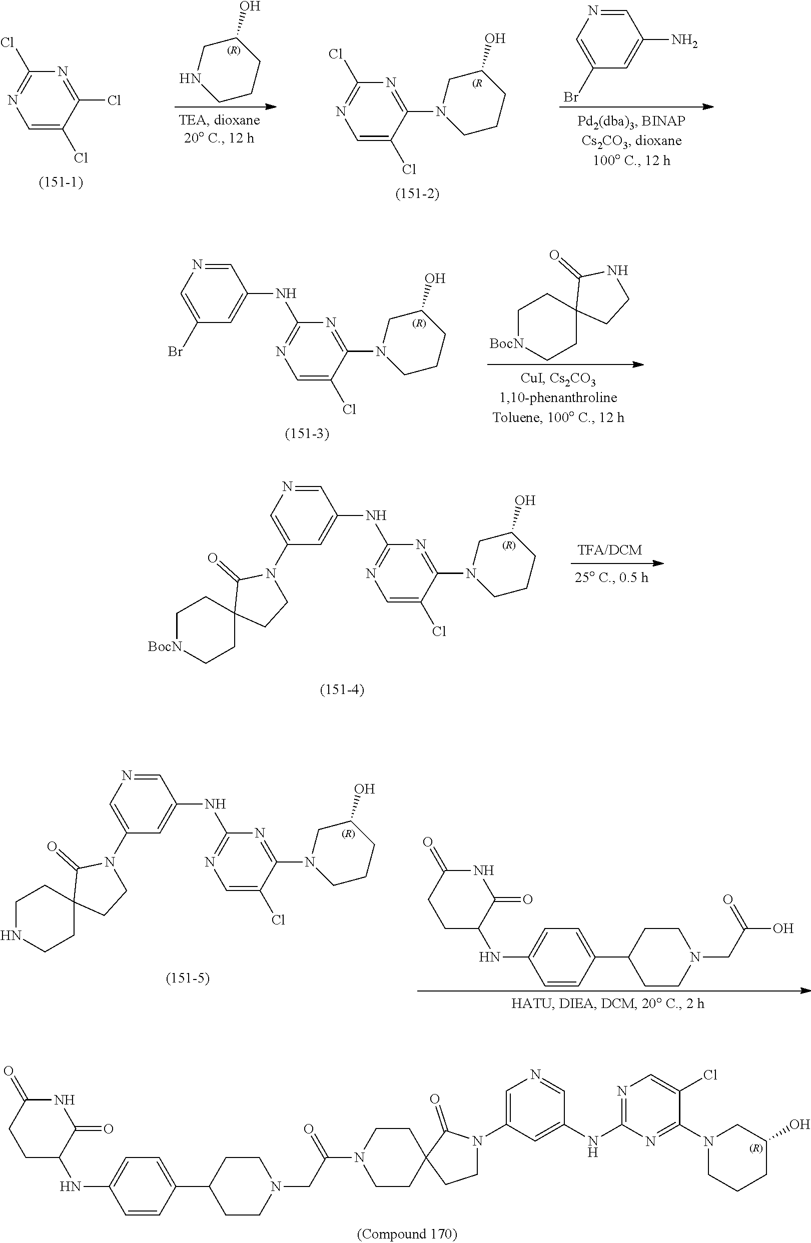

Synthesis of (R)-1-(2,5-dichloropyrimidin-4-yl)piperidin-3-ol (151-2). To a solution of 2,4,5-trichloropyrimidine (151-1) (1.63 g, 8.90 mmol, 1.00 eq) and (R)-piperidin-3-ol (900 mg, 8.90 mmol, 1.00 eq) in dioxane (5 mL) was added TEA (2.70 g, 26.7 mmol, 3.72 mL, 3.00 eq) at 20° C. The mixture was stirred at 20° C. for 12 h. LCMS showed 2,4,5-trichloropyrimidine (151-1) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in vacuo. The mixture was purified by prep-TLC (SiO₂, PE/EtOAc=1/1) to give the title compound (151-2) (1.80 g, 7.25 mmol, 81.5% yield) as a white solid. LCMS (ES⁺): m/z 248.0 [M+H]⁺.

Synthesis of (R)-1-(2-((5-bromopyridin-3-yl)amino)-5-chloropyrimidin-4-yl)piperidin-3-ol (151-3). To a solution of (R)-1-(2,5-dichloropyrimidin-4-yl)piperidin-3-ol (151-2) (1.80 g, 7.25 mmol, 1.00 eq) and 5-bromopyridin-3-amine (2.51 g, 14.5 mmol, 2.00 eq) in dioxane (8 mL) was added BINAP (452 mg, 725 umol, 0.100 eq), Cs₂CO₃ (4.73 g, 14.5 mmol, 2.00 eq) and Pd₂(dba)₃ (664 mg, 725 umol, 0.100 eq) at 20° C. The resulting mixture was stirred at 100° C. for 12 h under N₂. LCMS showed (R)-1-(2,5-dichloropyrimidin-4-yl)piperidin-3-ol (151-2) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in vacuo. The mixture was purified by MPLC (SiO₂, PE/EtOAc=0/1 to I/O) to give the title compound (151-3) (700 mg, 1.82 mmol, 25.1% yield) as a yellow solid. LCMS (ES⁺): m/z 384.0 [M+H]⁺.

Synthesis of tert-butyl (R)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (151-4). To a solution of (R)-1-(2-((5-bromopyridin-3-yl)amino)-5-chloropyrimidin-4-yl)piperidin-3-ol (151-3) (190 g, 494 umol, 1.00 eq) in Toluene (3 mL) was added CuI (28.2 mg, 148 umol, 0.300 eq) at 20° C., the mixture was stirred 15 min at 20° C., then the mixture was added tert-butyl 1-oxo-2,8-diazaspiro[4.5]decane-8-carboxylate (151 mg, 593 umol, 1.20 eq), 1,10-phenanthroline (53.4 mg, 296 umol, 0.600 eq) and Cs₂CO₃ (483 mg, 1.48 mmol, 3.00 eq) at 20° C. The mixture was stirred at 100° C. for 12 h under N₂ atmosphere. LCMS showed (R)-1-(2-((5-bromopyridin-3-yl)amino)-5-chloropyrimidin-4-yl)piperidin-3-ol (151-3) was consumed and the desired mass was detected. To the mixture was added H₂O (10 mL) and the mixture was extracted with EtOAc (10 mL*3), dried over Na₂SO₄, filtered and the filtrate was concentrated in vacuo. The mixture was purified by prep-TLC (SiO₂, PE/EtOAc=0/1) to give the title compound (151-4) (100 mg, 179 umol, 36.3% yield) as a yellow solid. LCMS (ES⁺): m/z 558.1 [M+H]⁺.

Synthesis of (R)-2-(5-((5-chloro-4-(3-hydroxypiperidin-1-yl)pyrimidin-2-yl)amino)pyridin-3-yl)-2,8-diazaspiro[4.5]decan-1-one (151-5). To a solution of tert-butyl (R)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (151-4) (100 mg, 179 umol, 1.00 eq) in DCM (2.5 mL) was added TFA (0.5 mL) at 20° C. The resulting mixture was stirred at 20° C. for 0.5 h. LC-MS showed tert-butyl (R)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (151-4) was consumed and the desired mass was detected. The mixture was concentrated in vacuo to give the title compound (151-5) (110 mg, crude, TFA) as a yellow oil. LCMS (ES⁺): m/z 458.1 [M+H]⁺.

Synthesis of 3-((4-(1-(2-(2-(5-((5-chloro-4-((R)-3-hydroxypiperidin-1-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 170). To a solution of 2-(4-(4-((2,6-dioxopiperidin-3-yl)amino)phenyl)piperidin-1-yl)acetic acid (60.4 mg, 175 umol, 1.00 eq) in DMF (2 mL) were added HATU (79.8 mg, 210 μmol, 1.20 eq) and DIEA (113 mg, 874 μmol, 152 μL, 5.00 eq) at 20° C., then (R)-2-(5-((5-chloro-4-(3-hydroxypiperidin-1-yl)pyrimidin-2-yl)amino)pyridin-3-yl)-2,8-diazaspiro[4.5]decan-1-one (151-5) (100 mg, 175 μmol, 1.00 eq, TFA) was added to the mixture. The mixture was stirred at 20° C. for 2 h. LCMS showed (R)-2-(5-((5-chloro-4-(3-hydroxypiperidin-1-yl)pyrimidin-2-yl)amino)pyridin-3-yl)-2,8-diazaspiro[4.5]decan-1-one (151-5) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in vacuo. The mixture was purified by acidic prep-HPLC (column: C18-6 100*30 mm*5 um; mobile phase: [water (FA)-ACN]; B %: 10%-40%, 8 min) to give the title compound (Compound 170) (18.6 mg, 22.2 umol, 12.7% yield, 99.1% purity, FA) as a white solid. LCMS (ES⁺): m/z 785.4 [M+H]⁺.

### Example 152. Synthesis of 3-((4-(1-(2-(2-(5-((5-chloro-4-((R)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 171)

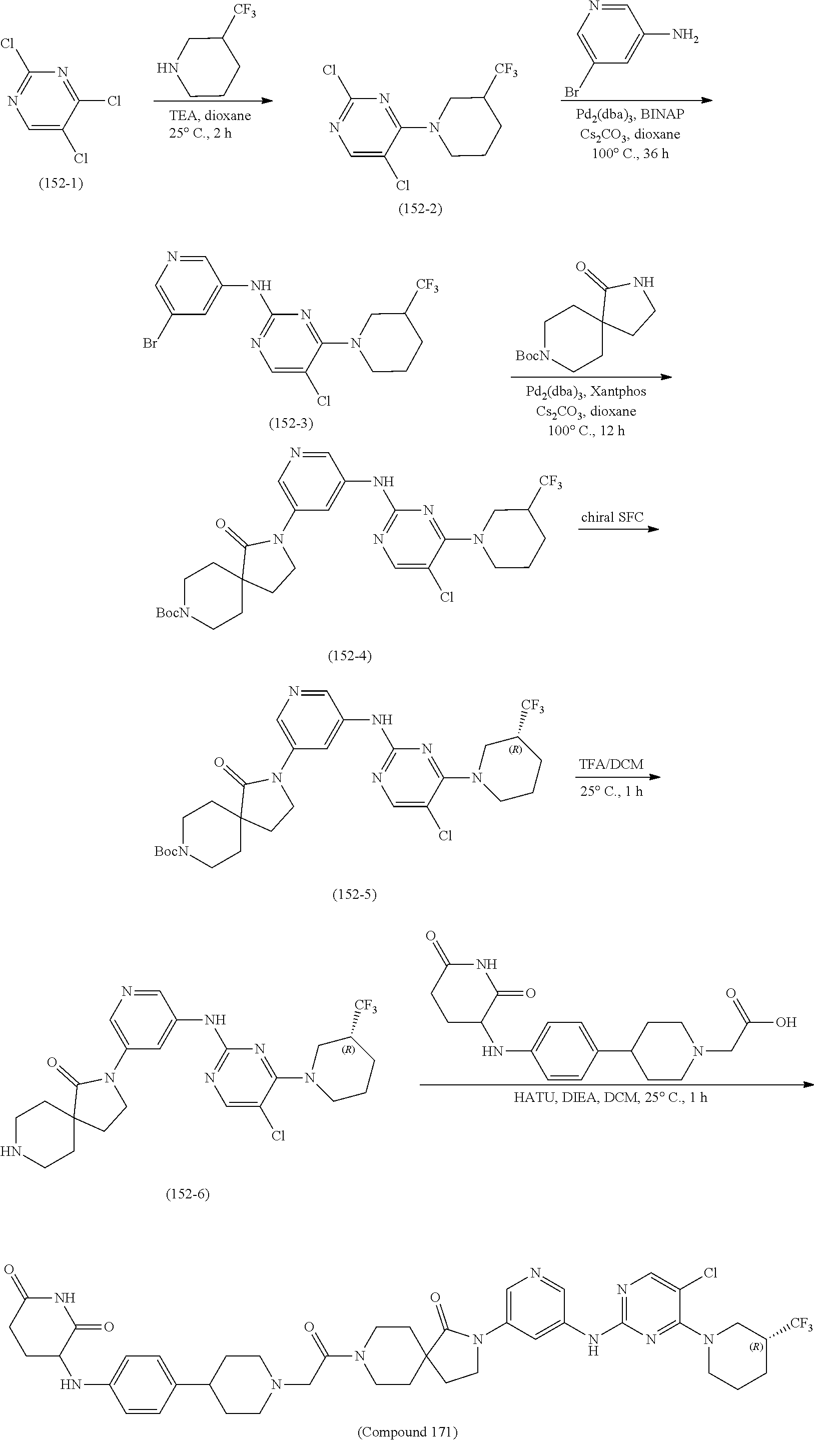

Synthesis of 2,5-dichloro-4-(3-(trifluoromethyl)piperidin-1-yl)pyrimidine (152-2). The reaction were set up two batches. To a solution of 2,4,5-trichloropyrimidine (152-1) (1.03 g, 5.45 mmol, 1.00 eq) in dioxane (15 mL) was added TEA (1.10 g, 10.9 mmol, 1.52 mL, 2.00 eq) at 25° C., then 3-(trifluoromethyl)piperidine (1.00 g, 5.45 mmol, 1.00 eq) was added to the mixture, the mixture was stirred at 25° C. for 2 h. LCMS showed 2,4,5-trichloropyrimidine (152-1) was consumed, desired target MS was detected. Two bathes in parallel were combined. The mixture was concentrated in vacuo, then H₂O (10 mL) was added to the mixture and the mixture was extracted with EtOAc (10 mL*3), the combined organic layers were washed with saturated aqueous NaCl solution (20 mL*3), dried over Na₂SO₄, filtered and the filtrate was concentrated in vacuo. The mixture was purified by MPLC (SiO₂, Petroleum ether/Ethyl acetate=1/0 to 5/1) to give the title compound (152-2) (2.20 g, 7.33 mmol, 67.2% yield) as a white solid. LCMS (ES⁺): m/z 299.9 [M+H]⁺.

Synthesis of N-(5-bromopyridin-3-yl)-5-chloro-4-(3-(trifluoromethyl)piperidin-1-yl)pyrimidin-2-amine (152-3). The reaction were set up to two parallels. To a solution of 2,5-dichloro-4-(3-(trifluoromethyl)piperidin-1-yl)pyrimidine (152-2) (1.00 g, 3.33 mmol, 1.00 eq) and 5-bromopyridin-3-amine (577 mg, 3.33 mmol, 1.00 eq) in dioxane (30 mL) were added BINAP (124 mg, 200 umol, 0.0600 eq), Pd₂(dba)₃ (122 mg, 133 umol, 0.0400 eq) and Cs₂CO₃ (2.17 g, 6.66 mmol, 2.00 eq) at 25° C. The mixture was stirred at 100° C. for 12 h under N₂ atmosphere. LCMS showed 2,5-dichloro-4-(3-(trifluoromethyl)piperidin-1-yl)pyrimidine (152-2) remained, desired target MS was detected. Then 5-bromopyridin-3-amine (577 mg, 3.33 mmol, 1.00 eq), BINAP (124 mg, 200 umol, 0.0600 eq), Pd₂(dba)₃ (122 mg, 133 umol, 0.0400 eq) was added to the mixture, the mixture was stirred at 100° C. for 12 h under N₂ atmosphere. LCMS showed 2,5-dichloro-4-(3-(trifluoromethyl)piperidin-1-yl)pyrimidine (152-2) was remained, desired target MS was detected. Then the mixture was stirred at 100° C. for 12 h under N₂ atmosphere. LCMS showed 2,5-dichloro-4-(3-(trifluoromethyl)piperidin-1-yl)pyrimidine (152-2) was consumed, desired target MS was detected. The two batches in parallel were combined. Then the combined mixture was concentrated in vacuo, then H₂O (20 mL) was added to the mixture and the mixture was extracted with EtOAc (30 mL*3), the combined organic layers were washed with saturated aqueous NaCl solution (30 mL*3), dried over Na₂SO₄, filtered and the filtrate was concentrated in vacuo to give the crude product. The crude product was purified by MPLC (SiO₂, PE/EtOAc=1/0 to 2/1) to give the title compound (152-3) (400 mg, 916 umol, 13.8% yield) as a yellow solid. LCMS (ES⁺): m/z 435.9 [M+H]⁺.

Synthesis of tert-butyl 2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (152-4). To a solution of N-(5-bromopyridin-3-yl)-5-chloro-4-(3-(trifluoromethyl)piperidin-1-yl)pyrimidin-2-amine (152-3) (330 mg, 756 μmol, 1.00 eq) and tert-butyl 1-oxo-2,8-diazaspiro[4.5]decane-8-carboxylate (192 mg, 756 μmol, 1.00 eq) in dioxane (5 mL) were added Pd₂(dba)₃ (20.8 mg, 22.7 μmol, 0.0300 eq), Cs₂CO₃ (492 mg, 1.51 mmol, 2.00 eq) and Xantphos (39.4 mg, 68.1 μmol, 0.0900 eq) at 25° C. The mixture was stirred at 100° C. for 12 h under N₂ atmosphere. LCMS showed N-(5-bromopyridin-3-yl)-5-chloro-4-(3-(trifluoromethyl)piperidin-1-yl)pyrimidin-2-amine (152-3) was consumed, desired target MS was detected. The mixture was concentrated in vacuo, then H₂O (5 mL) was added to the mixture and the mixture was extracted with EtOAc (5 mL*3), the combined organic layers were washed with saturated aqueous NaCl solution (5 mL*3), dried over Na₂SO₄, filtered and the filtrate was concentrated in vacuo. The crude product was purified by prep-TLC (SiO₂, EtOAc/MeOH=10/1) to give the title compound (152-4) (360 mg, crude) was obtained. LCMS (ES⁺): m/z 610.3 [M+H]⁺.

Synthesis of tert-butyl (R)-2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (152-5). 360 mg tert-butyl 2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (152-4) was separated by chiral SFC (CAS-TJ-ANA-Chiral HPLC-K (Waters Arc with 2998)); Column: Chiralpak IF-3, 100×4.6 mm I.D., 3 um; Mobile phase: A: Hexane B:EtOH+ACN (4:1)(0.1% IPAm, v/v); Gradient: A:B=50:50; Flow rate: 1.3 mL/min; Column temp.: 30° C.; Two enantiomers were assigned arbitrarily. The title compound (152-5) (123 mg, 175 μmol, 23.2% yield) was obtained as a yellow solid. LCMS (ES⁺): m/z 610.3 [M+H]⁺.

Synthesis of (R)-2-(5-((5-chloro-4-(3-(trifluoromethyl)piperidin-1-yl)pyrimidin-2-yl)amino)pyridin-3-yl)-2,8-diazaspiro[4.5]decan-1-one (152-6). A solution of tert-butyl (R)-2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (152-5) (60.0 mg, 98.4 μmol, 1.00 eq) in DCM (2 mL) and TFA (0.4 mL) was stirred at 25° C. for 1 h, LCMS showed tert-butyl (R)-2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (152-5) was consumed, desired target MS was detected. The mixture was concentrated in vacuo to give the title compound (152-6) (61.0 mg, crude, TFA) as a yellow oil. LCMS (ES⁺): m/z 510.4 [M+H]⁺.

Synthesis of 3-((4-(1-(2-(2-(5-((5-chloro-4-((R)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 171). To a solution of 2-(4-(4-((2,6-dioxopiperidin-3-yl)amino)phenyl)piperidin-1-yl)acetic acid (33.2 mg, 96.2 μmol, 1.00 eq) and (R)-2-(5-((5-chloro-4-(3-(trifluoromethyl)piperidin-1-yl)pyrimidin-2-yl)amino)pyridin-3-yl)-2,8-diazaspiro[4.5]decan-1-one (152-6) (60.0 mg, 96.2 μmol, 1.00 eq, TFA) in DMF (1 mL) were added HATU (43.9 mg, 115 μmol, 1.20 eq) and DIEA (62.1 mg, 481 μmol, 83.7 μL, 5.00 eq) at 25° C., the mixture was stirred at 25° C. for 1 h. LCMS showed 2-(4-(4-((2,6-dioxopiperidin-3-yl)amino)phenyl)piperidin-1-yl)acetic acid and (R)-2-(5-((5-chloro-4-(3-(trifluoromethyl)piperidin-1-yl)pyrimidin-2-yl)amino)pyridin-3-yl)-2,8-diazaspiro[4.5]decan-1-one (152-6) was consumed, desired target MS was detected, the mixture was concentrated in vacuo. The crude product was purified by prep-HPLC (column: Phenomenex Luna C18 75*30 mm*3 um; mobile phase: [water (FA)-ACN]; B %: 5%-40%, 8 min) to give the title compound (Compound 171) (28.5 mg, 34.0 μmol, 35.4% yield, 100% purity) as a white solid. LCMS (ES⁺): m/z 837.2 [M+H]⁺.

### Example 153. Synthesis of 3-((4-(1-(2-(2-(5-((5-chloro-4-((S)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 172)

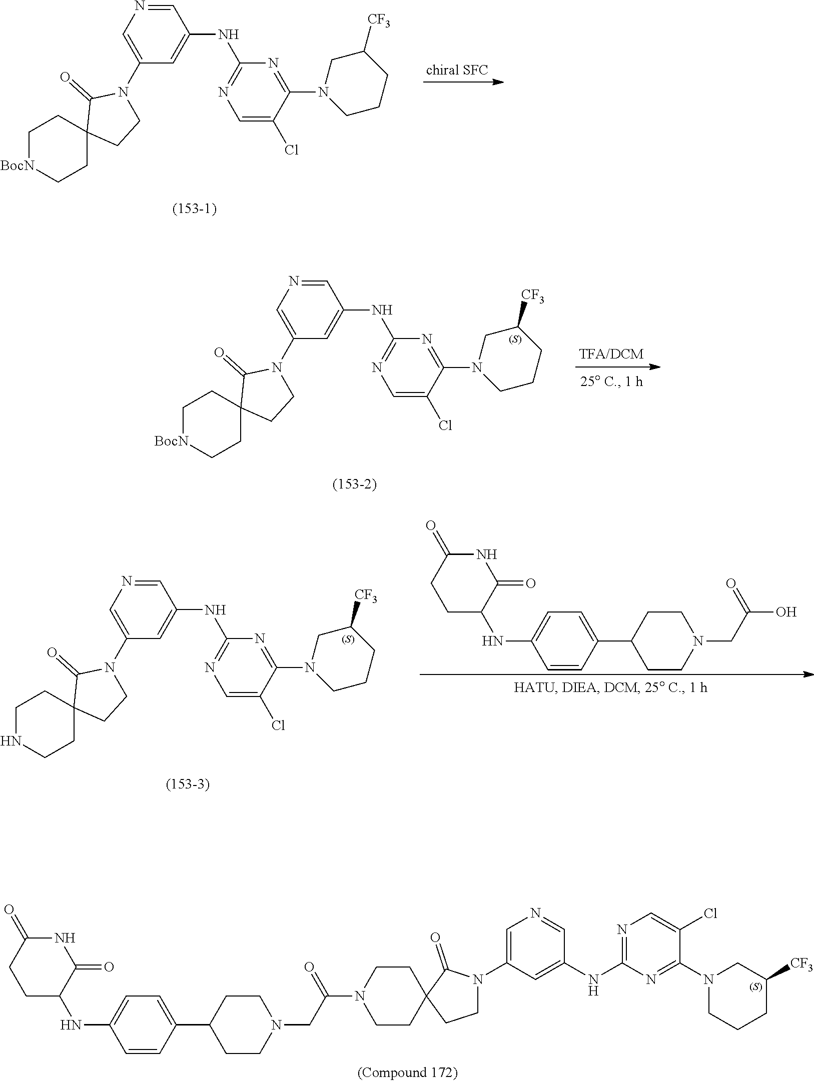

### Synthesis of tert-butyl (S)-2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (153-2)

360 mg tert-butyl 2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (153-1) was separated by chiral SFC (CAS-TJ-ANA-Chiral HPLC-K (Waters Arc with 2998)); Column: Chiralpak IF-3, 100×4.6 mm I.D., 3 um; Mobile phase: A: Hexane B:EtOH+ACN (4:1)(0.1% IPAm, v/v); Gradient: A:B=50:50; Flow rate: 1.3 mL/min; Column temp.: 30° C.; Two enantiomers were assigned arbitrarily. The title compound (153-2) (145 mg, 229 μmol, 30.3% yield) was obtained as a yellow solid. LCMS (ES⁺): m/z 610.4 [M+H]⁺.

Synthesis of (S)-2-(5-((5-chloro-4-(3-(trifluoromethyl)piperidin-1-yl)pyrimidin-2-yl)amino)pyridin-3-yl)-2,8-diazaspiro[4.5]decan-1-one (153-3). A solution of tert-butyl (S)-2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (153-2) (70 mg, 114.74 μmol, 1.00 eq) in DCM (2 mL) and TFA (0.4 mL) was stirred at 25° C. for 1 h, LCMS showed tert-butyl (S)-2-(5-((5-chloro-4-(3-(trifluoromethyl)piperidin-1-yl)pyrimidin-2-yl)amino)pyridin-3-yl)-1-oxo-2,8-diazaspiro[4.5]decane-8-carboxylate (153-2) was consumed, desired target MS was detected. The mixture was concentrated in vacuo to give the title compound (153-3) (70.0 mg, crude, TFA) as a yellow oil. LCMS (ES⁺): m/z 510.4 [M+H]⁺.

Synthesis of 3-((4-(1-(2-(2-(5-((5-chloro-4-((S)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 172). To a solution of 2-(4-(4-((2,6-dioxopiperidin-3-yl)amino)phenyl)piperidin-1-yl)acetic acid (33.2 mg, 96.2 μmol, 1.00 eq) and (S)-2-(5-((5-chloro-4-(3-(trifluoromethyl)piperidin-1-yl)pyrimidin-2-yl)amino)pyridin-3-yl)-2,8-diazaspiro[4.5]decan-1-one (153-3) (60 mg, 96.2 μmol, 1.00 eq, TFA) in DMF (1 mL) were added HATU (43.9 mg, 115 μmol, 1.20 eq) and DIEA (62.1 mg, 481 μmol, 83.7 μL, 5.00 eq) at 25° C., the mixture was stirred at 25° C. for 1 h. LCMS showed 2-(4-(4-((2,6-dioxopiperidin-3-yl)amino)phenyl)piperidin-1-yl)acetic acid and (S)-2-(5-((5-chloro-4-(3-(trifluoromethyl)piperidin-1-yl)pyrimidin-2-yl)amino)pyridin-3-yl)-2,8-diazaspiro[4.5]decan-1-one (153-3) was consumed, desired target MS was detected. The mixture was concentrated in vacuo. The crude product was purified by prep-HPLC (column: Phenomenex Luna C18 75*30 mm*3 um; mobile phase: [water (FA)-ACN]; B %: 5%-40%, 8 min) to give the title compound (Compound 172) (32.9 mg, 37.47 μmol, 39.0% yield, 95.4% purity) as a white solid. LCMS (ES⁺): m/z 837.3 [M+H]⁺.

### Example 154. Synthesis of 3-((4-(4-(2-(2-(5-((5-chloro-4-(piperidin-1-yl)pyrimidin-2-yl)amino)pyridin-3-yl)-1-oxo-2,8-diazaspiro[4.5]decan-8-yl)ethyl)piperidin-1-yl)phenyl)amino)piperidine-2,6-dione (Compound 173)

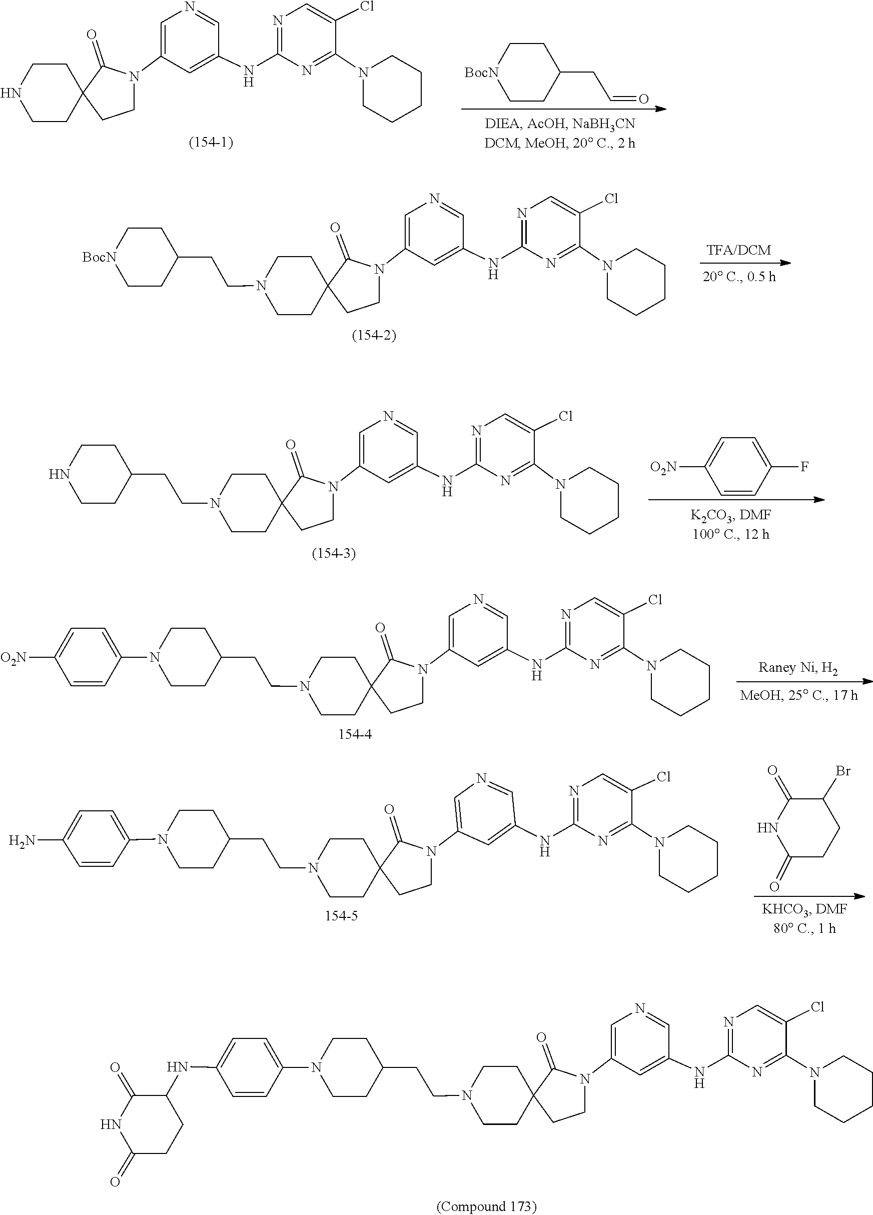

Synthesis of tert-butyl 4-(2-(2-(5-((5-chloro-4-(piperidin-1-yl)pyrimidin-2-yl)amino)pyridin-3-yl)-1-oxo-2,8-diazaspiro[4.5]decan-8-yl)ethyl)piperidine-1-carboxylate (154-2). To a solution of 2-(5-((5-chloro-4-(piperidin-1-yl)pyrimidin-2-yl)amino)pyridin-3-yl)-2,8-diazaspiro[4.5]decan-1-one (154-1) (2.00 g, 3.60 mmol, 1.00 eq, TFA) in DCM (20 mL) and MeOH (20 mL) was added DIEA (2.32 g, 18.0 mmol, 3.13 mL, 5.00 eq) at 20° C., the mixture was stirred at 20° C. for 0.5 h. Then tert-butyl 4-(2-oxoethyl)piperidine-1-carboxylate (2.45 g, 10.8 mmol, 3.00 eq) and AcOH (21.6 mg, 360 umol, 20.6 uL, 0.100 eq) were added to the mixture at 20° C., the mixture was stirred at 20° C. for 0.5 h. Then NaBH₃CN (678 mg, 10.8 mmol, 3.00 eq) was added to the mixture at 20° C., the mixture was stirred at 20° C. for 1 h. LCMS showed 2-(5-((5-chloro-4-(piperidin-1-yl)pyrimidin-2-yl)amino)pyridin-3-yl)-2,8-diazaspiro[4.5]decan-1-one (154-1) was consumed completely and the desired mass was detected. The reaction was concentrated under reduced pressure. Water (10 mL) was added to the mixture, then it was extracted with EtOAc (10 mL*3), the combined organic layers were dried over Na₂SO₄, filtered and the filtrate was concentrated under reduced pressure. The residue was purified by column chromatography (SiO₂, Ethyl acetate:Methanol=10:1) to give the title compound (154-2) (3.00 g, crude) as a yellow oil. LCMS (ES⁺): m/z 653.6 [M+H]⁺.

Synthesis of 2-(5-((5-chloro-4-(piperidin-1-yl)pyrimidin-2-yl)amino)pyridin-3-yl)-8-(2-(piperidin-4-yl)ethyl)-2,8-diazaspiro[4.5]decan-1-one (154-3). A solution of tert-butyl 4-(2-(2-(5-((5-chloro-4-(piperidin-1-yl)pyrimidin-2-yl)amino)pyridin-3-yl)-1-oxo-2,8-diazaspiro[4.5]decan-8-yl)ethyl)piperidine-1-carboxylate (154-2) (2.80 g, 4.29 mmol, 1.00 eq) in TFA (10 mL) and DCM (40 mL) was stirred at 20° C. for 0.5 h. LCMS showed tert-butyl 4-(2-(2-(5-((5-chloro-4-(piperidin-1-yl)pyrimidin-2-yl)amino)pyridin-3-yl)-1-oxo-2,8-diazaspiro[4.5]decan-8-yl)ethyl)piperidine-1-carboxylate (154-2) was consumed completely and the desired mass was detected. The reaction was concentrated under reduced pressure to give the title compound (154-3) (2.86 g, crude, TFA) as a yellow oil. LCMS (ES⁺): m/z 553.5 [M+H]⁺.

Synthesis of 2-(5-((5-chloro-4-(piperidin-1-yl)pyrimidin-2-yl)amino)pyridin-3-yl)-8-(2-(1-(4-nitrophenyl)piperidin-4-yl)ethyl)-2,8-diazaspiro[4.5]decan-1-one (154-4). To a solution of 2-(5-((5-chloro-4-(piperidin-1-yl)pyrimidin-2-yl)amino)pyridin-3-yl)-8-(2-(piperidin-4-yl)ethyl)-2,8-diazaspiro[4.5]decan-1-one (154-3) (2.66 g, 3.99 mmol, 1.00 eq, TFA) in DMF (50 mL) were added 1-fluoro-4-nitro-benzene (563 mg, 3.99 mmol, 423 μL, 1.00 eq) and K₂CO₃ (3.86 g, 27.9 mmol, 7.00 eq) at 20° C., the mixture was stirred at 100° C. for 12 h. LCMS showed 2-(5-((5-chloro-4-(piperidin-1-yl)pyrimidin-2-yl)amino)pyridin-3-yl)-8-(2-(piperidin-4-yl)ethyl)-2,8-diazaspiro[4.5]decan-1-one (154-3) was consumed completely and the desired mass was detected. Water (50 mL) was added to the mixture, then it was extracted with EtOAc (50 mL*3), the combined organic layers were dried over Na₂SO₄, filtered and the filtrate was concentrated under reduced pressure. The residue was purified by column chromatography (SiO₂, Petroleum ether:Ethyl acetate=1:0 to 0:1) to give the title compound (154-4) (1.40 g, 2.08 mmol, 52.1% yield) as a yellow solid. LCMS (ES⁺): m/z 674.6 [M+H]⁺.

Synthesis of 8-(2-(1-(4-aminophenyl)piperidin-4-yl)ethyl)-2-(5-((5-chloro-4-(piperidin-1-yl)pyrimidin-2-yl)amino)pyridin-3-yl)-2,8-diazaspiro[4.5]decan-1-one (154-5). To a solution of 2-(5-((5-chloro-4-(piperidin-1-yl)pyrimidin-2-yl)amino)pyridin-3-yl)-8-(2-(1-(4-nitrophenyl)piperidin-4-yl)ethyl)-2,8-diazaspiro[4.5]decan-1-one (154-4) (1.34 g, 1.99 mmol, 1.00 eq) in MeOH (20 mL) was added Raney-Ni (1.34 g) at 25° C. under H₂ atmosphere. The suspension was degassed and purged with H₂ for three times. The mixture was stirred under H₂ (15 Psi) at 25° C. for 5 h. LCMS showed 2-(5-((5-chloro-4-(piperidin-1-yl)pyrimidin-2-yl)amino)pyridin-3-yl)-8-(2-(1-(4-nitrophenyl)piperidin-4-yl)ethyl)-2,8-diazaspiro[4.5]decan-1-one (154-4) was remained mostly and the desired mass was detected. The mixture was stirred at 25° C. for 12 h. LCMS showed 2-(5-((5-chloro-4-(piperidin-1-yl)pyrimidin-2-yl)amino)pyridin-3-yl)-8-(2-(1-(4-nitrophenyl)piperidin-4-yl)ethyl)-2,8-diazaspiro[4.5]decan-1-one (154-4) was consumed completely and the desired mass was detected. The reaction mixture was filtered and the filtrated was concentrated under reduced pressure to give the title compound (154-5) (730 mg, crude) as a green solid. LCMS (ES⁺): m/z 644.3 [M+H]⁺.

Synthesis of 3-((4-(4-(2-(2-(5-((5-chloro-4-(piperidin-1-yl)pyrimidin-2-yl)amino)pyridin-3-yl)-1-oxo-2,8-diazaspiro[4.5]decan-8-yl)ethyl)piperidin-1-yl)phenyl)amino)piperidine-2,6-dione (Compound 173). To a solution of 3-bromopiperidine-2,6-dione (179 mg, 931 umol, 2.00 eq) in DMF (6 mL) were added KHCO₃ (140 mg, 1.40 mmol, 3.00 eq) and 8-(2-(1-(4-aminophenyl)piperidin-4-yl)ethyl)-2-(5-((5-chloro-4-(piperidin-1-yl)pyrimidin-2-yl)amino)pyridin-3-yl)-2,8-diazaspiro[4.5]decan-1-one (154-5) (300 mg, 466 umol, 1.00 eq) at 25° C. The mixture was stirred at 80° C. for 1 h. LCMS showed 8-(2-(1-(4-aminophenyl)piperidin-4-yl)ethyl)-2-(5-((5-chloro-4-(piperidin-1-yl)pyrimidin-2-yl)amino)pyridin-3-yl)-2,8-diazaspiro[4.5]decan-1-one (154-5) was consumed completely and the desired mass was detected. The organic layers were filtered and the filtrate was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 40%-70%, 8 min) to give the crude product. The crude product was purified by prep-HPLC (column: Waters Xbridge BEH C18 100*30 mm*10 um; mobile phase: [water (NH₄HCO₃)-ACN]; B %: 50%-80%, 8 min) to give the title compound (Compound 173) (3.30 mg, 4.21 μmol, 9.04e-1% yield, 96.4% purity) as a gray solid. LCMS (ES⁺): m/z 755.3 [M+H]⁺.

### Example 155. Synthesis of 3-((4-(4-((2-(5-((5-chloro-4-(4-chlorophenyl)pyrimidin-2-yl)amino)pyridin-3-yl)-1-oxo-2,8-diazaspiro[4.5]decan-8-yl)methyl)piperidin-1-yl)phenyl)amino)piperidine-2,6-dione (Compound 174)

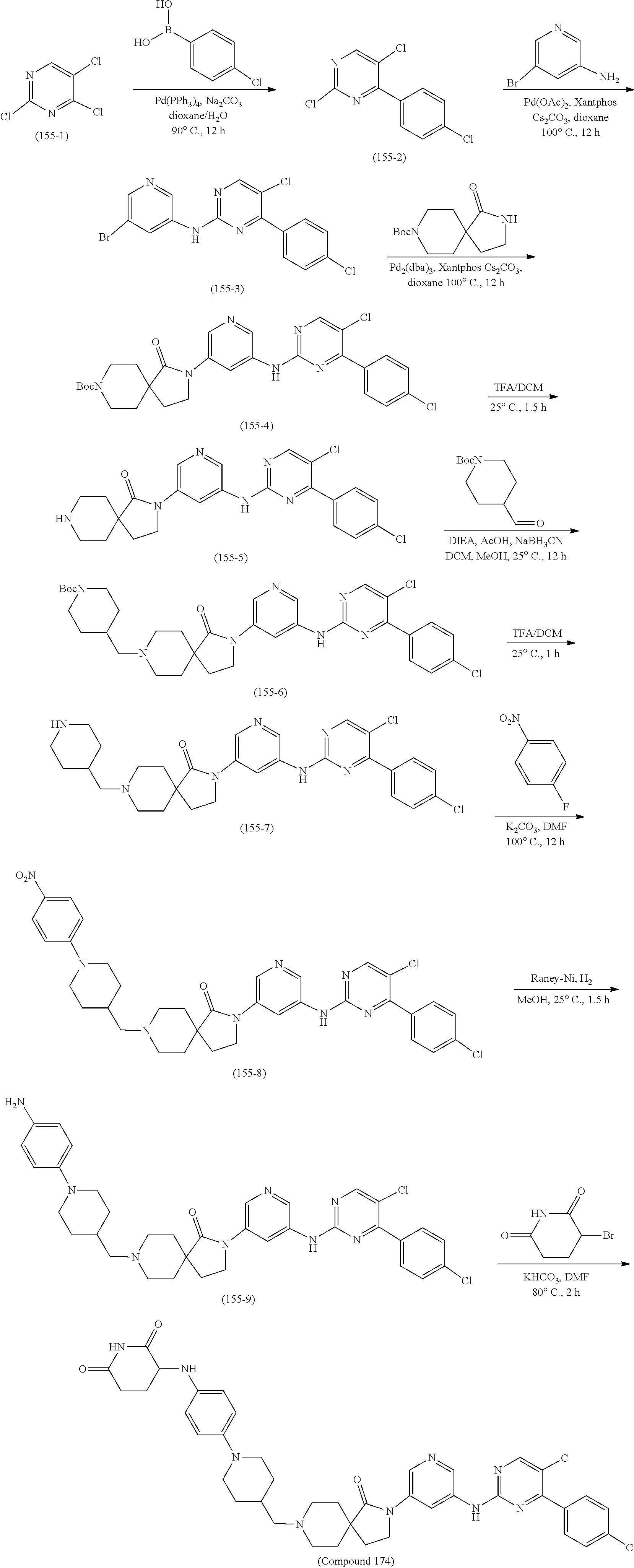

Synthesis of 2,5-dichloro-4-(4-chlorophenyl)pyrimidine (155-2). To a solution of 2,4,5-trichloropyrimidine (155-1) (70.4 g, 384 mmol, 1.20 eq) in dioxane (1000 mL) and H₂O (100 mL) were added Pd(PPh₃)₄ (7.39 g, 6.39 mmol, 0.0200 eq), (4-chlorophenyl)boronic acid (50.0 g, 320 mmol, 1.00 eq) and Na₂CO₃ (67.8 g, 640 mmol, 2.00 eq) at 25° C. under N₂ atmosphere. The mixture was stirred at 90° C. for 12 h under N₂ atmosphere. LCMS showed that the desired mass was detected. The reaction mixture was concentrated under reduced pressure to remove solvent. The residue was purified by column chromatography (SiO₂, Petroleum ether/Ethyl acetate=1/0 to 50/1) to give the title compound (155-2) (72.4 g, 243 mmol, 75.9% yield, 87.0% purity) as a white solid. LCMS (ES⁺): m/z 258.9 [M+H]⁺

Synthesis of N-(5-bromopyridin-3-yl)-5-chloro-4-(4-chlorophenyl)pyrimidin-2-amine (155-3). To a solution of 2,5-dichloro-4-(4-chlorophenyl)pyrimidine (155-2) (60.0 g, 201 mmol, 87.0% purity, 1.00 eq) in dioxane (1000 mL) were added Pd(OAc)₂ (3.16 g, 14.1 mmol, 0.0700 eq), Cs₂CO₃ (164 g, 503 mmol, 2.50 eq), 5-bromopyridin-3-amine (34.8 g, 201 mmol, 1.00 eq) and Xantphos (5.82 g, 10.1 mmol, 0.0500 eq) at 25° C. under N₂ atmosphere. The mixture was stirred at 100° C. for 12 h under N₂ atmosphere. LCMS showed that 2,5-dichloro-4-(4-chlorophenyl)pyrimidine (155-2) was consumed completely the desired mass was detected. Water (1000 mL) was added to the mixture at 25° C. Then the reaction mixture was extracted with EtOAc (800 mL*3). The combined organic layers were dried over Na₂SO₄, filtered and the filtrate was concentrated under reduced pressure to give a residue. The residue was purified by column chromatography (SiO₂, Petroleum ether/Ethyl acetate=10/1 to 3/1) to give the title compound (155-3) (45.0 g, 114 mmol, 56.5% yield) as a brown solid. LCMS (ES⁺): m/z 395.0 [M+H]⁺

Synthesis of tert-butyl 2-(5-((5-chloro-4-(4-chlorophenyl)pyrimidin-2-yl)amino)pyridin-3-yl)-1-oxo-2,8-diazaspiro[4.5]decane-8-carboxylate (155-4). To a solution of N-(5-bromo-3-pyridyl)-5-chloro-4-(4-chlorophenyl)pyrimidin-2-amine (155-3) (3.00 g, 7.57 mmol, 1.00 eq) and tert-butyl 1-oxo-2,8-diazaspiro[4.5]decane-8-carboxylate (1.54 g, 6.06 mmol, 0.800 eq) in dioxane (50 mL) were added Xantphos (394 mg, 682 umol, 0.0900 eq), Cs₂CO₃ (4.94 g, 15.2 mmol, 2.00 eq) and Pd₂(dba)₃ (208 mg, 227 umol, 0.0300 eq) at 25° C. under N₂ atmosphere. The resulting mixture was stirred at 100° C. for 12 h under N₂ atmosphere. LCMS showed that N-(5-bromo-3-pyridyl)-5-chloro-4-(4-chlorophenyl)pyrimidin-2-amine (155-3) was consumed completely and the desired mass was detected. The mixture was diluted with H₂O (40 mL) and then the aqueous was extracted with EtOAc (30 mL*3). The combined organic layers were dried over Na₂SO₄, filtered and the filtrate was concentrated under reduced pressure. The mixture was purified by column chromatography (SiO₂, PE/EtOAc=1/1 to I/O) to give the title compound (155-4) (2.40 g, 4.21 mmol, 55.6% yield) as a yellow solid. LCMS (ES⁺): m/z 513.1 [M+H−56]⁺

Synthesis of 2-(5-((5-chloro-4-(4-chlorophenyl)pyrimidin-2-yl)amino)pyridin-3-yl)-2,8-diazaspiro[4.5]decan-1-one (155-5). To a solution of tert-butyl 2-(5-((5-chloro-4-(4-chlorophenyl)pyrimidin-2-yl)amino)pyridin-3-yl)-1-oxo-2,8-diazaspiro[4.5]decane-8-carboxylate (155-4) (1.50 g, 2.63 mmol, 1.00 eq) in DCM (15 mL) was added TFA (7.70 g, 67.5 mmol, 5 mL, 25.6 eq) at 25° C. The mixture was stirred at 25° C. for 1.5 h. LCMS showed that tert-butyl 2-(5-((5-chloro-4-(4-chlorophenyl)pyrimidin-2-yl)amino)pyridin-3-yl)-1-oxo-2,8-diazaspiro[4.5]decane-8-carboxylate (155-4) was consumed completely and the desired mass was detected. The reaction mixture was concentrated under reduced pressure to remove solvent to give the title compound (155-5) (1.54 g, crude, TFA) as an orange red oil. LCMS (ES⁺): m/z 469.2 [M+H]⁺

Synthesis of tert-butyl 4-((2-(5-((5-chloro-4-(4-chlorophenyl)pyrimidin-2-yl)amino)pyridin-3-yl)-1-oxo-2,8-diazaspiro[4.5]decan-8-yl)methyl)piperidine-1-carboxylate (155-6). To a solution of 2-(5-((5-chloro-4-(4-chlorophenyl)pyrimidin-2-yl)amino)pyridin-3-yl)-2,8-diazaspiro[4.5]decan-1-one (155-5) (1.54 g, 2.64 mmol, 1.00 eq, TFA) in DCM (15 mL) and MeOH (15 mL) was added DIEA (1.71 g, 13.2 mmol, 2.30 mL, 5.00 eq) at 25° C., the mixture was stirred at 25° C. for 0.5 h. Then tert-butyl 4-formylpiperidine-1-carboxylate (1.69 g, 7.92 mmol, 3.00 eq) and AcOH (15.9 mg, 264 umol, 15.1 uL, 0.100 eq) were added to the mixture at 25° C., then the mixture was stirred at 25° C. for 0.5 h. Then NaBH₃CN (498 mg, 7.92 mmol, 3.00 eq) was added to the mixture at 25° C., the mixture was stirred at 25° C. for 11 h. LCMS showed 2-(5-((5-chloro-4-(4-chlorophenyl)pyrimidin-2-yl)amino)pyridin-3-yl)-2,8-diazaspiro[4.5]decan-1-one (155-5) was consumed completely and the desired mass was detected. H₂O (30 mL) was added to the mixture at 25° C. The reaction mixture was extracted with DCM (15 mL*3), the organic layers were dried over anhydrous sodium sulfate, filtered and the filtrate was concentrated under reduced to give a residue. The residue was purified by column chromatography (SiO₂, Petroleum ether/Ethyl acetate=10/1 to 5/1) to give the title compound (155-6) (1.18 g, 1.77 mmol, 67.1% yield) as a light yellow solid. LCMS (ES⁺): m/z 666.4 [M+H]⁺.

Synthesis of 2-(5-((5-chloro-4-(4-chlorophenyl)pyrimidin-2-yl)amino)pyridin-3-yl)-8-(piperidin-4-ylmethyl)-2,8-diazaspiro[4.5]decan-1-one (155-7). To a solution of tert-butyl 4-((2-(5-((5-chloro-4-(4-chlorophenyl)pyrimidin-2-yl)amino)pyridin-3-yl)-1-oxo-2,8-diazaspiro[4.5]decan-8-yl)methyl)piperidine-1-carboxylate (155-6) (500 mg, 750 umol, 1.00 eq) in DCM (12 mL) was added TFA (6.16 g, 54.0 mmol, 4.00 mL, 72.0 eq) at 25° C. The mixture was stirred at 25° C. for 1 h. LCMS showed tert-butyl 4-((2-(5-((5-chloro-4-(4-chlorophenyl)pyrimidin-2-yl)amino)pyridin-3-yl)-1-oxo-2,8-diazaspiro[4.5]decan-8-yl)methyl)piperidine-1-carboxylate (155-6) was consumed completely and the desired mass was detected. The reaction mixture was concentrated under reduced pressure to remove solvent to give the title compound (155-7) (510 mg, crude, TFA) as a yellow oil. LCMS (ES⁺): m/z 566.2 [M+H]⁺.

Synthesis of 2-(5-((5-chloro-4-(4-chlorophenyl)pyrimidin-2-yl)amino)pyridin-3-yl)-8-((1-(4-nitrophenyl)piperidin-4-yl)methyl)-2,8-diazaspiro[4.5]decan-1-one (155-8). To a solution of 2-(5-((5-chloro-4-(4-chlorophenyl)pyrimidin-2-yl)amino)pyridin-3-yl)-8-(piperidin-4-ylmethyl)-2,8-diazaspiro[4.5]decan-1-one (155-7) (510 mg, 750 umol, 1.00 eq, TFA) in DMF (6 mL) were added 1-fluoro-4-nitro-benzene (106 mg, 750 umol, 79.6 uL, 1.00 eq) and K₂CO₃ (207 mg, 1.50 mmol, 2.00 eq) at 25° C. The mixture was stirred at 100° C. for 12 h. LCMS showed ˜3% of 2-(5-((5-chloro-4-(4-chlorophenyl)pyrimidin-2-yl)amino)pyridin-3-yl)-8-(piperidin-4-ylmethyl)-2,8-diazaspiro[4.5]decan-1-one (155-7) was remaining, and ˜60% of the desired mass was detected. H₂O (10 mL) was added to the mixture at 25° C. The reaction mixture was extracted with EtOAc (10 mL*4). The organic layers were dried over anhydrous sodium sulfate, filtered and the filtrate was concentrated under reduced to give a residue. The residue was purified by prep-TLC (SiO₂, Ethyl acetate:Methanol=1:2) to give the title compound (155-8) (213 mg, 310 umol, 41.3% yield) as a yellow oil. LCMS (ES⁺): m/z: 687.2 [M+H]⁺.

Synthesis of 8-((1-(4-aminophenyl)piperidin-4-yl)methyl)-2-(5-((5-chloro-4-(4-chlorophenyl)pyrimidin-2-yl)amino)pyridin-3-yl)-2,8-diazaspiro[4.5]decan-1-one (155-9). To a solution of 2-(5-((5-chloro-4-(4-chlorophenyl)pyrimidin-2-yl)amino)pyridin-3-yl)-8-((1-(4-nitrophenyl)piperidin-4-yl)methyl)-2,8-diazaspiro[4.5]decan-1-one (155-8) (200 mg, 291 μmol, 1.00 eq) in MeOH (20 mL) was added Raney-Ni (250 mg) at 25° C., the suspension was degassed and purged with H₂ for three times. The mixture was stirred at 25° C. for 1.5 h under H₂ (15 psi) atmosphere. LCMS showed 2-(5-((5-chloro-4-(4-chlorophenyl)pyrimidin-2-yl)amino)pyridin-3-yl)-8-((1-(4-nitrophenyl)piperidin-4-yl)methyl)-2,8-diazaspiro[4.5]decan-1-one (155-8) was consumed completely and the desired mass was detected. The reaction mixture was filtered and the filtrate was concentrated under reduced to remove the solvent to give a residue. The residue was purified by prep-HPLC (column: Waters Xbridge Prep OBD C18 150*40 mm*10 um; mobile phase: [water (NH₄HCO₃)-ACN]; B %: 40%-75%, 8 min) to give the title compound (155-9) (20.0 mg, 30.4 μmol, 10.5% yield) as a brown solid. LCMS (ES⁺): m/z: 657.4 [M+H]⁺.

Synthesis of 3-((4-(4-((2-(5-((5-chloro-4-(4-chlorophenyl)pyrimidin-2-yl)amino)pyridin-3-yl)-1-oxo-2,8-diazaspiro[4.5]decan-8-yl)methyl)piperidin-1-yl)phenyl)amino)piperidine-2,6-dione (Compound 174). To a solution of 8-((1-(4-aminophenyl)piperidin-4-yl)methyl)-2-(5-((5-chloro-4-(4-chlorophenyl)pyrimidin-2-yl)amino)pyridin-3-yl)-2,8-diazaspiro[4.5]decan-1-one (155-9) (15.0 mg, 22.8 μmol, 1.00 eq) in DMF (1 mL) were added KHCO₃ (6.85 mg, 68.4 μmol, 3.00 eq) and 3-bromopiperidine-2,6-dione (8.76 mg, 45.6 μmol, 2.00 eq) at 25° C. The mixture was stirred at 80° C. for 1 h. LCMS showed ˜5% of 8-((1-(4-aminophenyl)piperidin-4-yl)methyl)-2-(5-((5-chloro-4-(4-chlorophenyl)pyrimidin-2-yl)amino)pyridin-3-yl)-2,8-diazaspiro[4.5]decan-1-one (155-9) was remaining, and ˜84% of the desired mass was detected. To the mixture were added KHCO₃ (6.85 mg, 68.4 μmol, 3.00 eq) and 3-bromopiperidine-2,6-dione (8.76 mg, 45.6 μmol, 2.00 eq) at 25° C. The mixture was stirred at 80° C. for another 1 h. LCMS showed 8-((1-(4-aminophenyl)piperidin-4-yl)methyl)-2-(5-((5-chloro-4-(4-chlorophenyl)pyrimidin-2-yl)amino)pyridin-3-yl)-2,8-diazaspiro[4.5]decan-1-one (155-9) was consumed completely and the desired mass was detected. The reaction mixture was concentrated under reduced pressure to remove solvent. The residue was purified by prep-HPLC (column: Waters Xbridge Prep OBD C18 150*40 mm*10 um; mobile phase: [water (NH₄HCO₃)-ACN]; B %: 40%-65%, 8 min) to give the title compound (Compound 174) (2.00 mg, 2.60 μmol, 11.4% yield, 100% purity) as a white solid. LCMS (ES⁺): m/z 768.2 [M+H]⁺.

### Example 156. Synthesis of 3-((4-(4-(2-(2-(5-((5-chloro-4-((S)-3-hydroxypiperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 175)

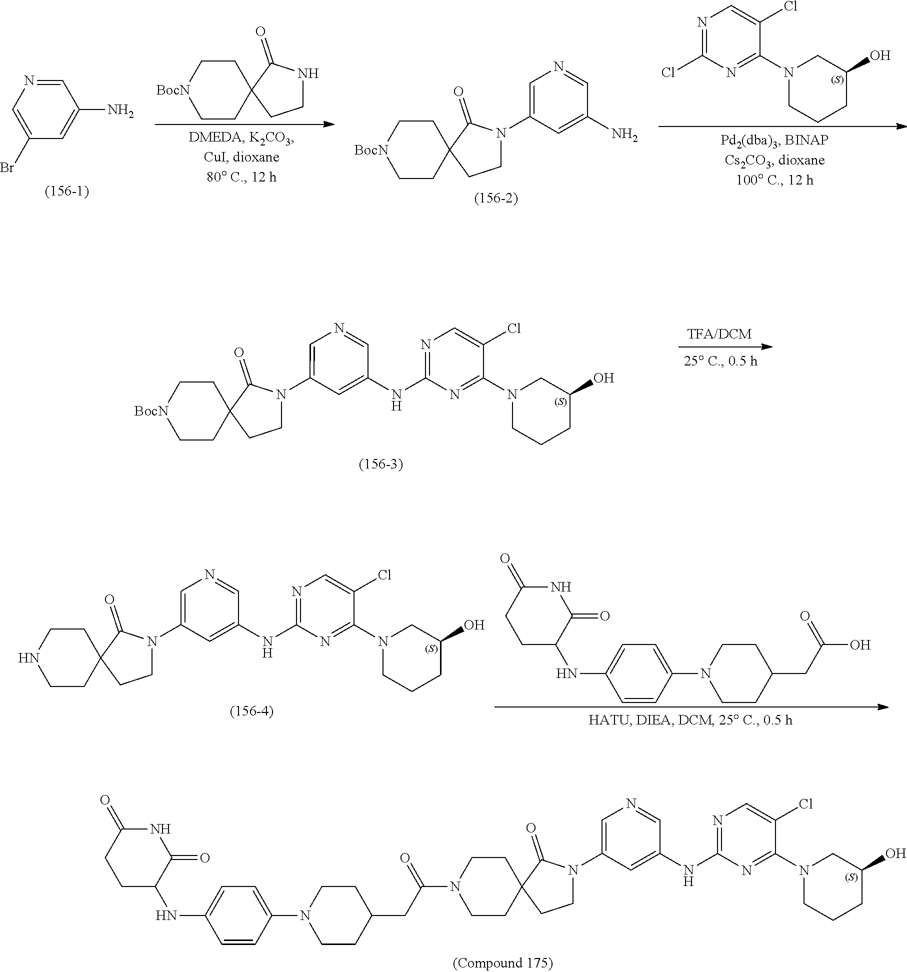

Synthesis of tert-butyl 2-(5-aminopyridin-3-yl)-1-oxo-2,8-diazaspiro[4.5]decane-8-carboxylate (156-2). Two batches in parallel were set up. To a solution of 5-bromopyridin-3-amine (156-1) (2.50 g, 14.5 mmol, 1.00 eq) and tert-butyl 1-oxo-2,8-diazaspiro[4.5]decane-8-carboxylate (3.67 g, 14.5 mmol, 1.00 eq) in dioxane (30 mL) was added CuI (275 mg, 1.44 mmol, 0.100 eq) and DMEDA (255 mg, 2.89 mmol, 311 μL, 0.200 eq) and K₂CO₃ (3.99 g, 28.9 mmol, 2.00 eq) at 25° C. The mixture was stirred at 80° C. for 12 h under N₂. LCMS showed 5-bromopyridin-3-amine (156-1) was consumed, desired target MS was detected. Two batches in parallel were combined. H₂O (50 mL) was added to the solution, the mixture was extracted with EtOAc (50 mL*3), then the combined organic layers were dried over Na₂SO₄, then concentrated in vacuo. The mixture was purified by MPLC (SiO₂, Petroleum ether:Ethyl acetate=1/0 to 0/1) to give the title compound (156-2) (1.60 g, 4.62 mmol, 16.0% yield) as a yellow solid. LCMS (ES⁺): m/z 347.1 [M+H]⁺.

Synthesis of tert-butyl (S)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (156-3). To a solution of (S)-1-(2,5-dichloropyrimidin-4-yl)piperidin-3-ol (500 mg, 2.02 mmol, 1.00 eq) and tert-butyl 2-(5-aminopyridin-3-yl)-1-oxo-2,8-diazaspiro[4.5]decane-8-carboxylate (156-2) (698 mg, 2.02 mmol, 1.00 eq) in dioxane (5 mL) were added Cs₂CO₃ (1.31 g, 4.03 mmol, 2.00 eq), BINAP (125 mg, 202 μmol, 0.100 eq) and Pd₂(dba)₃ (185 mg, 202 μmol, 0.100 eq) at 25° C., the mixture was stirred at 100° C. for 12 h under N₂ atmosphere. LCMS showed tert-butyl 2-(5-aminopyridin-3-yl)-1-oxo-2,8-diazaspiro[4.5]decane-8-carboxylate (156-2) was consumed mostly, desired target MS was detected. H₂O (5 mL) was added to the solution, the mixture was extracted with EtOAc (5 mL*3), then the combined organic lays were dried over Na₂SO₄, then concentrated in vacuo. The mixture was purified by prep-TLC (SiO₂, PE/EtOAc=0/1) to give the title compound (156-3) (1.06 g, 1.90 mmol, 94.3% yield) as a brown oil. LCMS (ES⁺): m/z 558.2 [M+H]⁺.

Synthesis of (S)-2-(5-((5-chloro-4-(3-hydroxypiperidin-1-yl)pyrimidin-2-yl)amino)pyridin-3-yl)-2,8-diazaspiro[4.5]decan-1-one (156-4). A solution of tert-butyl (S)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (156-3) (150 mg, 269 μmol, 1.00 eq) in TFA (0.7 mL) and DCM (2 mL) was stirred at 25° C. for 0.5 h. LCMS showed tert-butyl (S)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (156-3) was consumed completely, desired target MS was detected. The mixture was concentrated in vacuo to give the title compound (156-4) (153 mg, crude, TFA) as a yellow oil. LCMS (ES⁺): m/z 458.2 [M+H]⁺.

Synthesis of 3-((4-(4-(2-(2-(5-((5-chloro-4-((S)-3-hydroxypiperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 175). To a solution of 2-(1-(4-((2,6-dioxopiperidin-3-yl)amino)phenyl)piperidin-4-yl)acetic acid (115 mg, 2505 μmol, 1.00 eq, TFA) in DMF (1 mL) were added HATU (114 mg, 300 μmol, 1.20 eq), DIEA (162 mg, 1.25 mmol, 218 μL, 5.00 eq) at 25° C., then (S)-2-(5-((5-chloro-4-(3-hydroxypiperidin-1-yl)pyrimidin-2-yl)amino)pyridin-3-yl)-2,8-diazaspiro[4.5]decan-1-one (156-4) (143 mg, 250 μmol, 1.00 eq, TFA) was added to the mixture, the mixture was stirred at 25° C. for 0.5 h. LCMS showed (S)-2-(5-((5-chloro-4-(3-hydroxypiperidin-1-yl)pyrimidin-2-yl)amino)pyridin-3-yl)-2,8-diazaspiro[4.5]decan-1-one (156-4) was consumed, desired target MS was detected. The mixture was concentrated in vacuo. The residue was purified by prep-HPLC (column: Phenomenex Luna C18 200*40 mm*10 um; mobile phase: [water (FA)-ACN]; B %: 10%-40%, 8 min) to give the title compound (Compound 175) (50.4 mg, 60.5 μmol, 24.2% yield, 94.3% purity) as a white solid. LCMS (ES⁺): m/z 785.3 [M+H]⁺.

### Example 157. Synthesis of 3-((4-(4-(2-(2-(5-((5-chloro-4-((R)-3-hydroxypiperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 176)

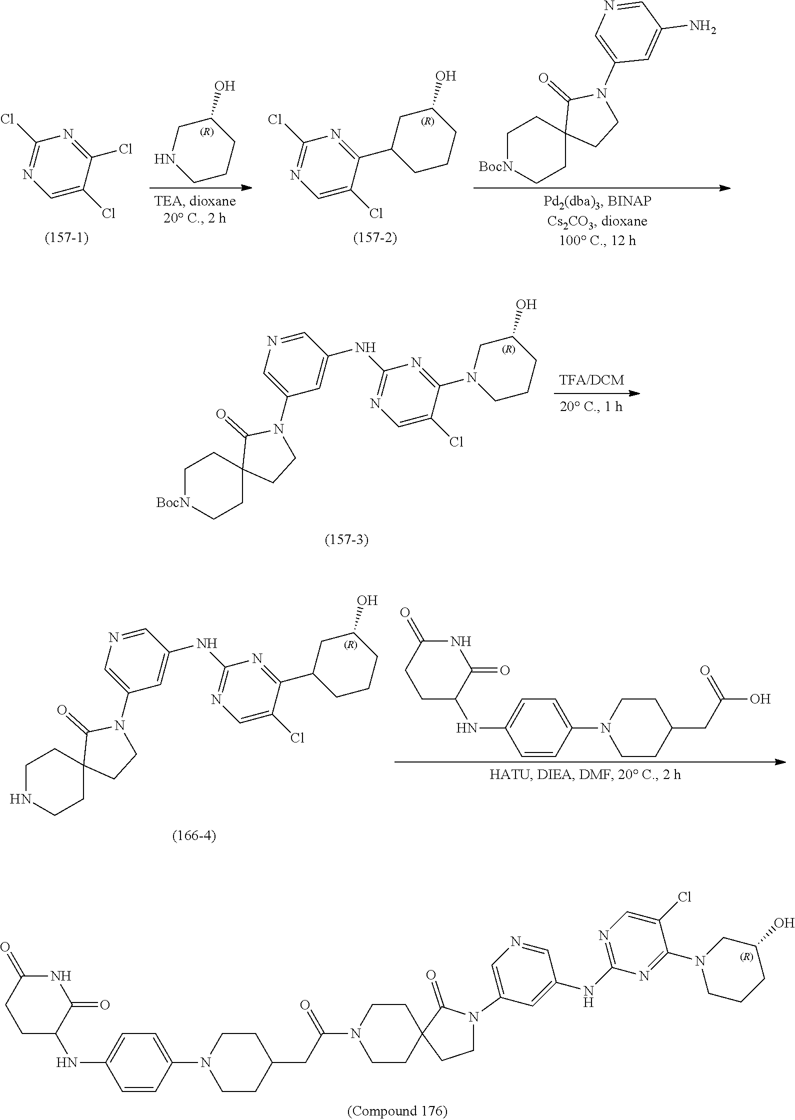

Synthesis of (R)-1-(2,5-dichloropyrimidin-4-yl)piperidin-3-ol (157-2). To a solution of 2,4,5-trichloropyrimidine (157-1) (1.81 g, 9.89 mmol, 1.00 eq) and (R)-piperidin-3-ol (1.00 g, 9.89 mmol, 1.00 eq) in dioxane (8 mL) and TEA (3.00 g, 29.7 mmol, 4.13 mL, 3.00 eq) at 20° C. The mixture was stirred at 20° C. for 2 h. LCMS showed 2,4,5-trichloropyrimidine (157-1) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in vacuo. The mixture was purified by MPLC (SiO₂, PE/EtOAc=1/0 to 1/1) to give the title compound (157-2) (1.79 g, 7.21 mmol, 73.0% yield) as a white solid. LCMS (ES⁺): m/z 248.0 [M+H]⁺.

Synthesis of tert-butyl (R)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (157-3). To a solution of (R)-1-(2,5-dichloropyrimidin-4-yl)piperidin-3-ol (157-2) (500 mg, 2.02 mmol, 1.00 eq) and tert-butyl 2-(5-aminopyridin-3-yl)-1-oxo-2,8-diazaspiro[4.5]decane-8-carboxylate (698 mg, 2.02 mmol, 1.00 eq) in dioxane (8 mL) were added BINAP (125 mg, 202 umol, 0.100 eq), Cs₂CO₃ (1.31 g, 4.03 mmol, 2.00 eq) and Pd₂(dba)₃ (185 mg, 202 umol, 0.100 eq) at 20° C. The resulting mixture was stirred at 100° C. for 12 h under N₂. LC-MS showed (R)-1-(2,5-dichloropyrimidin-4-yl)piperidin-3-ol (157-2) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in vacuo. The mixture was purified by MPLC (SiO₂, Dichloromethane/Methanol=10/1) to give the title compound (157-3) (420 mg, 753 umol, 37.3% yield) as a yellow solid. LCMS (ES⁺): m/z 558.2 [M+H]⁺.

Synthesis of (R)-2-(5-((5-chloro-4-(3-hydroxypiperidin-1-yl)pyrimidin-2-yl)amino)pyridin-3-yl)-2,8-diazaspiro[4.5]decan-1-one (157-4). To a solution of tert-butyl (R)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (157-3) (250 mg, 448 umol, 1.00 eq) in DCM (3 mL) was added TFA (0.6 mL) at 20° C. The resulting mixture was stirred at 20° C. for 1 h. LCMS showed tert-butyl (R)-2-(5-((5-chloro-4-(3-hydroxypiperidin-1-yl)pyrimidin-2-yl)amino)pyridin-3-yl)-1-oxo-2,8-diazaspiro[4.5]decane-8-carboxylate (157-3) was consumed and the desired mass was detected. The mixture was concentrated in vacuo to give the title compound (157-4) (240 mg, crude, TFA) as a yellow oil. LCMS (ES⁺): m/z 458.1 [M+H]⁺.

Synthesis of 3-((4-(4-(2-(2-(5-((5-chloro-4-((R)-3-hydroxypiperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 176). To a solution of 2-(1-(4-((2,6-dioxopiperidin-3-yl)amino)phenyl)piperidin-4-yl)acetic acid (145 mg, 420 umol, 1.00 eq) in DMF (3 mL) were added HATU (191 mg, 504 umol, 1.20 eq) and DIEA (271 mg, 2.10 mmol, 365 μL, 5.00 eq) at 20° C., then (R)-2-(5-((5-chloro-4-(3-hydroxypiperidin-1-yl)pyrimidin-2-yl)amino)pyridin-3-yl)-2,8-diazaspiro[4.5]decan-1-one (157-4) (240 mg, 420 umol, 1.00 eq, TFA) was added to the mixture. The mixture was stirred at 20° C. for 2 h. LCMS showed (R)-2-(5-((5-chloro-4-(3-hydroxypiperidin-1-yl)pyrimidin-2-yl)amino)pyridin-3-yl)-2,8-diazaspiro[4.5]decan-1-one (157-4) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in vacuo. The mixture was purified by acidic prep-HPLC (column: Phenomenex Luna 80*30 mm*3 um; mobile phase: [water (FA)-ACN]; B %: 10%-40%, 8 min) to give the title compound (Compound 176) (37.6 mg, 46.6 umol, 11.1% yield, 97.4% purity) as a gray solid. LCMS (ES⁺): m/z 785.3 [M+H]⁺.

### Example 158. Synthesis of 3-((4-(4-(2-(2-(5-((5-chloro-4-((R)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 177)

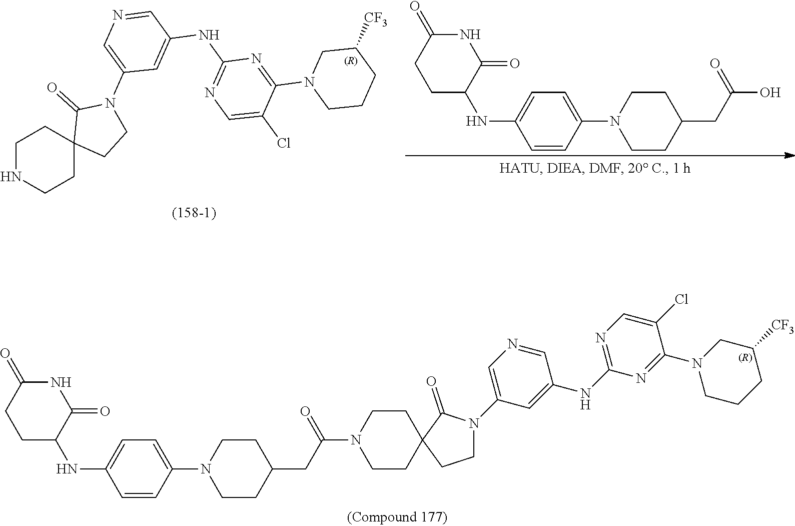

Synthesis of 3-((4-(4-(2-(2-(5-((5-chloro-4-((R)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 177). To a solution of 2-(1-(4-((2,6-dioxopiperidin-3-yl)amino)phenyl)piperidin-4-yl)acetic acid (33.2 mg, 96.2 μmol, 1.00 eq) and (R)-2-(5-((5-chloro-4-(3-(trifluoromethyl)piperidin-1-yl)pyrimidin-2-yl)amino)pyridin-3-yl)-2,8-diazaspiro[4.5]decan-1-one (158-1) (60.0 mg, 96.2 μmol, 1.00 eq, TFA) in DMF (1 mL) were added HATU (43.9 mg, 115 μmol, 1.20 eq) and DIEA (62.1 mg, 480 μmol, 83.7 μL, 5.00 eq) at 25° C., the mixture was stirred at 25° C. for 1 h. LCMS showed (R)-2-(5-((5-chloro-4-(3-(trifluoromethyl)piperidin-1-yl)pyrimidin-2-yl)amino)pyridin-3-yl)-2,8-diazaspiro[4.5]decan-1-one (158-1) was consumed, desired target MS was detected. The mixture was concentrated in vacuo. The crude product was purified by prep-HPLC (column: Phenomenex Luna 80*30 mm*3 um; mobile phase: [water (FA)-ACN]; B %: 10%-40%, 8 min) to give the title compound (Compound 177) (17.4 mg, 20.7 μmol, 21.6% yield, 99.8% purity) as a gray solid. LCMS (ES⁺): m/z 837.3 [M+H]⁺.

### Example 159. Synthesis of 3-((4-(4-(2-(2-(5-((5-chloro-4-((S)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 178)

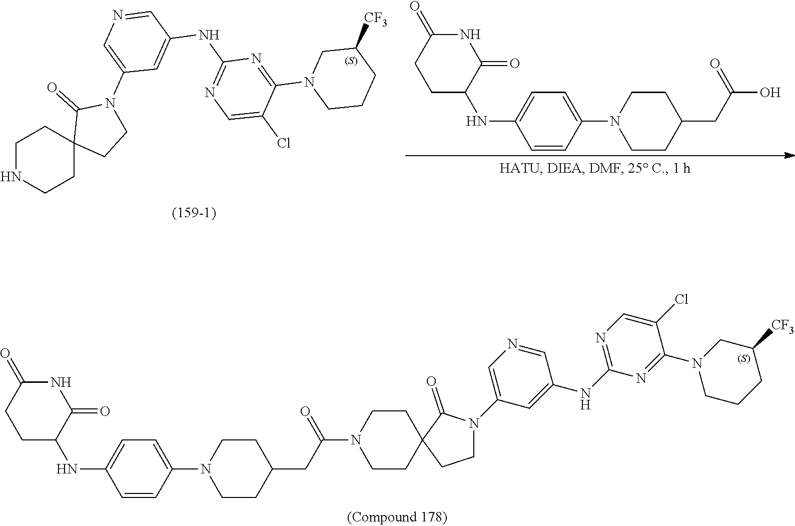

Synthesis of 3-((4-(4-(2-(2-(5-((5-chloro-4-((S)-3-(trifluoromethyl)piperidin-1-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 178). To a solution of 2-(1-(4-((2,6-dioxopiperidin-3-yl)amino)phenyl)piperidin-4-yl)acetic acid (33.2 mg, 72.3 μmol, 1.00 eq) and (S)-2-(5-((5-chloro-4-(3-(trifluoromethyl)piperidin-1-yl)pyrimidin-2-yl)amino)pyridin-3-yl)-2,8-diazaspiro[4.5]decan-1-one (159-1) (60.0 mg, 96.2 μmol, 1.00 eq, TFA) in DMF (1 mL) were added HATU (43.9 mg, 115 μmol, 1.20 eq) and DIEA (62.1 mg, 480 μmol, 83.7 μL, 5.00 eq) at 25° C., the mixture was stirred at 25° C. for 1 h. LCMS showed (S)-2-(5-((5-chloro-4-(3-(trifluoromethyl)piperidin-1-yl)pyrimidin-2-yl)amino)pyridin-3-yl)-2,8-diazaspiro[4.5]decan-1-one (159-1) was consumed, desired target MS was detected. The mixture was concentrated in vacuo. The residue was purified by prep-HPLC (column: Phenomenex Luna 80*30 mm*3 um; mobile phase: [water (FA)-ACN]; B %: 15%-45%, 8 min) to give the title compound (Compound 178) (2.60 mg, 3.11 μmol, 3.23% yield, 100% purity) as a gray solid. LCMS (ES⁺): m/z 837.3 [M+H]⁺.

### Example 160. Synthesis of 3-((4-(1-(2-(2-(5-((5-chloro-4-(3-(pyrrolidin-1-yl)phenyl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 179)

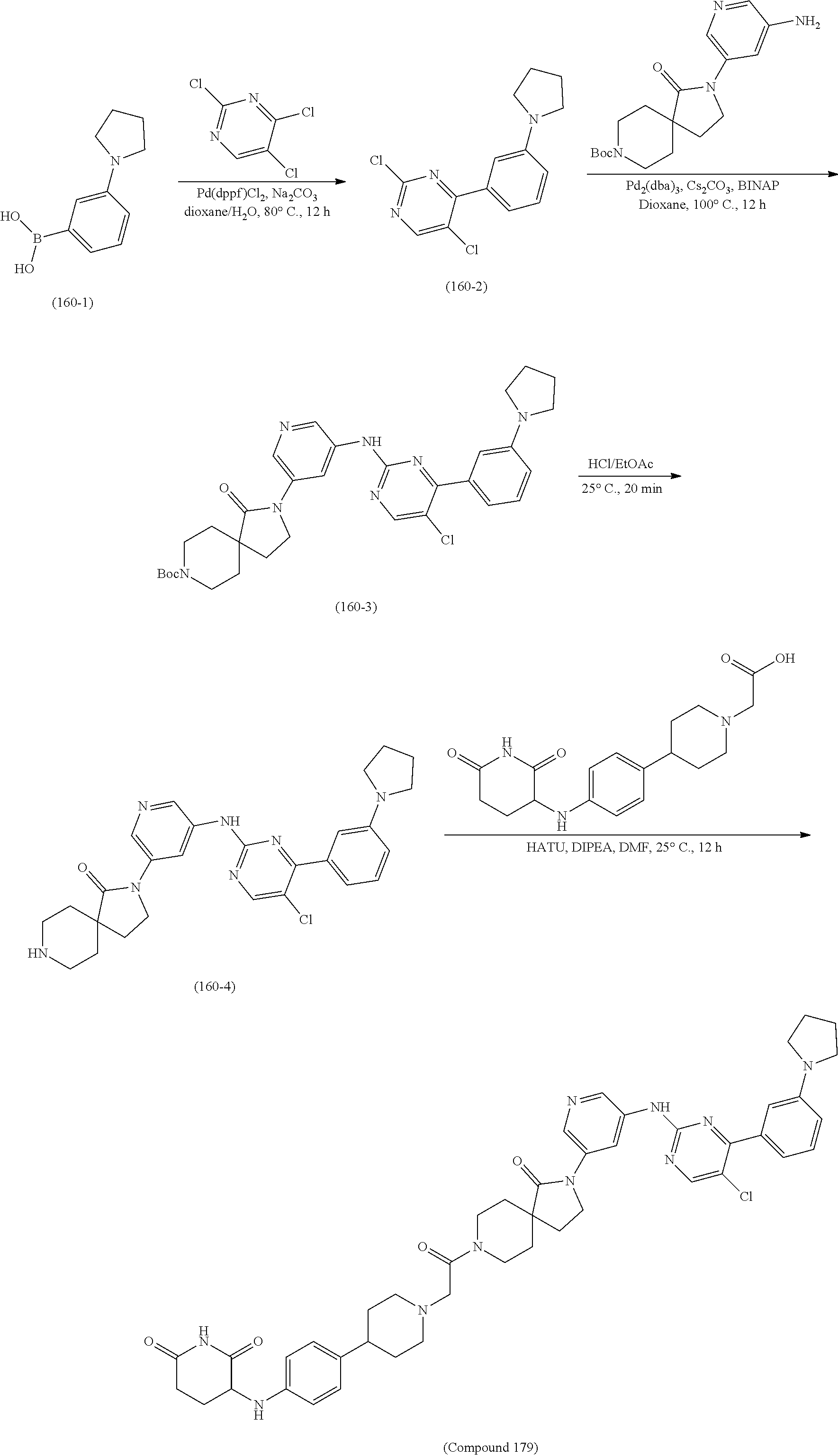

Synthesis of 2,5-dichloro-4-(3-(pyrrolidin-1-yl)phenyl)pyrimidine (160-2). To a solution of (3-(pyrrolidin-1-yl)phenyl)boronic acid (160-1) (2.00 g, 10.4 mmol, 1.00 eq), 2,4,5-trichloropyrimidine (2.88 g, 15.7 mmol, 1.50 eq), Na₂CO₃ (3.33 g, 31.4 mmol, 3.00 eq) and Pd(dppf)Cl₂ (766 mg, 1.05 mmol, 0.100 eq) in 1,4-Dioxane (10 mL) and H₂O (2 mL) was degassed and purged with N₂ at 25° C. for 3 times, and then the mixture was stirred at 80° C. for 12 h under N₂ atmosphere. LCMS showed (3-(pyrrolidin-1-yl)phenyl)boronic acid (160-1) was consumed completely and desired mass was detected. H₂O 10 mL was added to the mixture, and extracted with EtOAc (10 mL*3). The combined organic layers were washed with brine (10 mL), dried over Na₂SO₄, filtered and the filtrate was concentrated under reduced pressure. The residue was purified by prep-TLC (SiO₂, Petroleum ether/Ethyl acetate=5/1, R_{f}=0.6) to give the title compound (160-2)(1.3 g, crude) as a yellow oil. LCMS (ES⁺): m/z 294.0 [M+H]⁺.

Synthesis of tert-butyl 2-(5-((5-chloro-4-(3-(pyrrolidin-1-yl)phenyl)pyrimidin-2-yl)amino)pyridin-3-yl)-1-oxo-2,8-diazaspiro[4.5]decane-8-carboxylate (160-3). To a mixture of 2,5-dichloro-4-(3-(pyrrolidin-1-yl)phenyl)pyrimidine (160-2) (400 mg, 1.36 mmol, 1.00 eq), tert-butyl 2-(5-aminopyridin-3-yl)-1-oxo-2,8-diazaspiro[4.5]decane-8-carboxylate (565 mg, 1.63 mmol, 1.20 eq), Pd₂(dba)₃ (124 mg, 135 umol, 0.100 eq), BINAP (84.67 mg, 135.97 umol, 0.100 eq) and Cs₂CO₃ (1.33 g, 4.08 mmol, 3.00 eq) in dioxane (8 mL) at 25° C. was degassed and purged with N₂ for 3 times, and then the mixture was stirred at 100° C. for 12 h under N₂ atmosphere. LCMS showed 2,5-dichloro-4-(3-(pyrrolidin-1-yl)phenyl)pyrimidine (160-2) was consumed completely and the desired mass was detected. H₂O 10 mL was added to the mixture, and extracted with EtOAc (8 mL*3). The combined organic layers were washed with brine (10 mL*1), dried over Na₂SO₄, filtered and concentrated under reduced pressure. The residue was purified by column chromatography (SiO₂, Petroleum ether/Ethyl acetate=5/1, R_{f}=0.43) to give the title compound (160-3) (320 mg, 211 umol, 15.5% yield, 39.8% purity) as a white solid. LCMS (ES⁺): m/z 604.3 [M+H]⁺.

Synthesis of 2-(5-((5-chloro-4-(3-(pyrrolidin-1-yl)phenyl)pyrimidin-2-yl)amino)pyridin-3-yl)-2,8-diazaspiro[4.5]decan-1-one (160-4). A solution of tert-butyl 2-(5-((5-chloro-4-(3-(pyrrolidin-1-yl)phenyl)pyrimidin-2-yl)amino)pyridin-3-yl)-1-oxo-2,8-diazaspiro[4.5]decane-8-carboxylate (160-3) (150 mg, 248 μmol, 1.00 eq) in HCl/EtOAc (4 M, 5 mL) was stirred at 25° C. for 20 min. LCMS showed tert-butyl 2-(5-((5-chloro-4-(3-(pyrrolidin-1-yl)phenyl)pyrimidin-2-yl)amino)pyridin-3-yl)-1-oxo-2,8-diazaspiro[4.5]decane-8-carboxylate (160-3) was consumed completely and the desired mass was detected. The reaction mixture was concentrated under vacuum to give the title compound (160-4) (150 mg, crude) as a yellow solid. LCMS (ES⁺): m/z 504.3 [M+H]⁺.

Synthesis of 3-((4-(1-(2-(2-(5-((5-chloro-4-(3-(pyrrolidin-1-yl)phenyl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 179). To a solution of 2-(5-((5-chloro-4-(3-(pyrrolidin-1-yl)phenyl)pyrimidin-2-yl)amino)pyridin-3-yl)-2,8-diazaspiro[4.5]decan-1-one (160-4) (120 mg, 238 μmol, 1.00 eq) in DMF (1 mL) were added HATU (135 mg, 357 μmol, 1.50 eq), 2-(4-(4-((2,6-dioxopiperidin-3-yl)amino)phenyl)piperidin-1-yl)acetic acid (98.6 mg, 285 μmol, 1.20 eq) and DIEA (153 mg, 1.19 mmol, 207 μL, 5.00 eq) at 25° C. The mixture was stirred at 25° C. for 12 h. LCMS showed 2-(5-((5-chloro-4-(3-(pyrrolidin-1-yl)phenyl)pyrimidin-2-yl)amino)pyridin-3-yl)-2,8-diazaspiro[4.5]decan-1-one (160-4) was consumed completely and the desired mass was detected. The reaction mixture was filtered and the filtrate concentrated under high vacuum. The residue was purified by prep-HPLC (Phenomenex Luna C18 200*40 mm*10 um; mobile phase: [water (FA)-ACN]; B %: 10%-40%, 8 min) to give the title compound (Compound 179) (16.1 mg, 19.1 μmol, 8.04% yield, 100% purity, 0.2FA) as a yellow solid. LCMS (ES⁺): m/z 831.3 [M+H]⁺.

### Example 161. Synthesis of 3-[4-[4-[2-[2-[5-[[5-chloro-4-(3-phenylphenyl)pyrimidin-2-yl]amino]-3-pyridyl]-1-oxo-2,8-diazaspiro[4.5]decan-8-yl]-2-oxo-ethyl]-4-hydroxy-1-piperidyl]anilino]piperidine-2,6-dione (Compound 180)

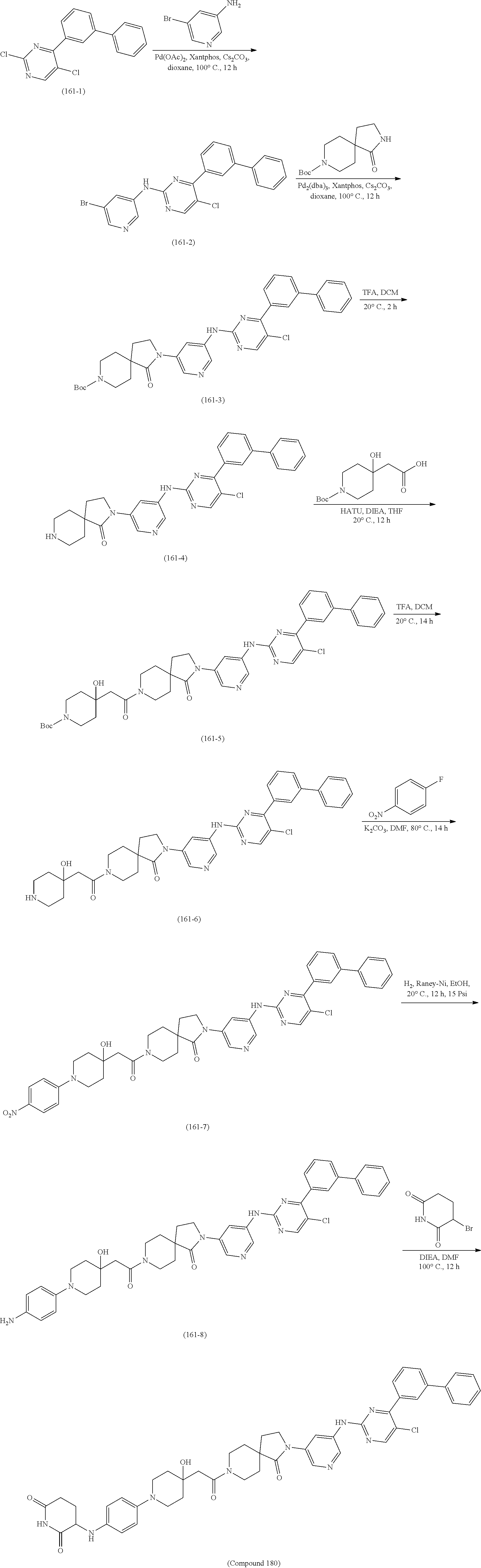

Synthesis of N-(5-bromo-3-pyridyl)-5-chloro-4-(3-phenylphenyl)pyrimidin-2-amine (161-2). The reaction was set up in 2 parallel batches. To a solution of 2,5-dichloro-4-(3-phenylphenyl)pyrimidine (161-1) (5.00 g, 16.6 mmol, 1.00 eq) in dioxane (100 mL) were added Pd(OAc)₂ (261 mg, 1.16 mmol, 0.0700 eq) and Xantphos (481 mg, 831 umol, 5.01e-2 eq), Cs₂CO₃ (13.5 g, 41.4 mmol, 2.50 eq), 5-bromopyridin-3-amine (2.87 g, 16.6 mmol, 9.99e-1 eq) at 20° C. under N₂. The mixture was stirred at 100° C. for 12 h. LCMS showed that 2,5-dichloro-4-(3-phenylphenyl)pyrimidine (161-1) was remained and one main peak with the desired mass was detected. 2 batches were combined to work up. The mixture was filtered and the filtrate was concentrated under reduced pressure. The residue was purified by column chromatography (SiO₂, Petroleum ether/Ethyl acetate=0/1 to 3/1) to give the title compound (161-2) (5.70 g, 13.0 mmol, 39.2% yield) as a brown solid. LCMS (ES⁺): m/z 436.9 [M+1]⁺.

Synthesis of tert-butyl 2-[5-[[5-chloro-4-(3-phenylphenyl)pyrimidin-2-yl]amino]-3-pyridyl]-1-oxo-2,8-diazaspiro[4.5]decane-8-carboxylate (161-3). To a solution of N-(5-bromo-3-pyridyl)-5-chloro-4-(3-phenylphenyl)pyrimidin-2-amine (161-2) (5.10 g, 11.7 mmol, 1.00 eq) in dioxane (100 mL) were added Xantphos (607 mg, 1.05 mmol, 0.0900 eq) and Cs₂CO₃ (4.55 g, 13.98 mmol, 1.20 eq), Pd₂(dba)₃ (320 mg, 349 umol, 0.0300 eq), tert-butyl 1-oxo-2,8-diazaspiro[4.5]decane-8-carboxylate (2.96 g, 12.0 mmol, 1.00 eq) at 20° C. The mixture was stirred at 100° C. for 12 h under N₂. LCMS showed N-(5-bromo-3-pyridyl)-5-chloro-4-(3-phenylphenyl)pyrimidin-2-amine (161-2) was consumed completely and one main peak with the desired mass was detected. The mixture was filtered and the filtrated was quenched by addition H₂O (200 ml) at 25° C., and then extracted with EtOAc (100 mL*5). The combined organic layers were washed with brine (50 mL), dried over Na₂SO₄, filtered and concentrated under reduced pressure to give a residue. The residue was purified by column chromatography (SiO₂, Petroleum ether/THF=1/0 to 1/1) to give the title compound (161-3) (6.85 g, 11.2 mmol, 96.2% yield) as a brown solid. LCMS (ES⁺): m/z 611.2 [M+H]⁺.

Synthesis of 2-[5-[[5-chloro-4-(3-phenylphenyl)pyrimidin-2-yl]amino]-3-pyridyl]-2,8-diazaspiro[4.5]decan-1-one (161-4). A solution of tert-butyl 2-[5-[[5-chloro-4-(3-phenylphenyl)pyrimidin-2-yl]amino]-3-pyridyl]-1-oxo-2,8-diazaspiro[4.5]decane-8-carboxylate (161-3) (5.85 g, 9.57 mmol, 1.00 eq) in DCM (60 mL) was added TFA (30.8 g, 270 mmol, 20.0 mL, 28.2 eq) at 20° C. The mixture was stirred at 20° C. for 2 h. LCMS showed that tert-butyl 2-[5-[[5-chloro-4-(3-phenylphenyl)pyrimidin-2-yl]amino]-3-pyridyl]-1-oxo-2,8-diazaspiro[4.5]decane-8-carboxylate (161-3) was consumed completely and one main peak with the desired mass was detected. The mixture was concentrated under reduced pressure to give the title compound (161-4) (8.50 g, crude, TFA) as a brown oil. LCMS (ES⁺): m/z 511.1 [M+H]⁺.

Synthesis of tert-butyl 4-[2-[2-[5-[[5-chloro-4-(3-phenylphenyl)pyrimidin-2-yl]amino]-3-pyridyl]-1-oxo-2,8-diazaspiro[4.5]decan-8-yl]-2-oxo-ethyl]-4-hydroxy-piperidine-1-carboxylate (161-5). To a solution of 2-[5-[[5-chloro-4-(3-phenylphenyl)pyrimidin-2-yl]amino]-3-pyridyl]-2,8-diazaspiro[4.5]decan-1-one (161-4) (1.00 g, 1.60 mmol, 1.00 eq, TFA) in THE (10 mL) were added HATU (1.82 g, 4.79 mmol, 3.00 eq), 2-(1-tert-butoxycarbonyl-4-hydroxy-4-piperidyl)acetic acid (415 mg, 1.60 mmol, 1.00 eq) and DIEA (1.04 g, 8.03 mmol, 1.40 mL, 5.02 eq) at 20° C. The mixture was stirred at 20° C. for 12 h. LCMS showed 2-[5-[[5-chloro-4-(3-phenylphenyl)pyrimidin-2-yl]amino]-3-pyridyl]-2,8-diazaspiro[4.5]decan-1-one (161-4) was consumed completely and one main peak with the desired mass was detected. The mixture was filtered and the filtrate was concentrated under reduced pressure. The residue was purified by column chromatography (SiO₂, Petroleum ether/THF=1/0 to 1/2) to give the title compound (161-5) (1.20 g, 1.60 mmol, 100% yield) as a yellow oil. LCMS (ES⁺): m/z 752.3 [M+H]⁺.

Synthesis of 2-[5-[[5-chloro-4-(3-phenylphenyl)pyrimidin-2-yl]amino]-3-pyridyl]-8-[2-(4-hydroxy-4-piperidyl)acetyl]-2,8-diazaspiro[4.5]decan-1-one (161-6). To a solution of tert-butyl 4-[2-[2-[5-[[5-chloro-4-(3-phenylphenyl)pyrimidin-2-yl]amino]-3-pyridyl]-1-oxo-2,8-diazaspiro[4.5]decan-8-yl]-2-oxo-ethyl]-4-hydroxy-piperidine-1-carboxylate (161-5) (1.20 g, 1.60 mmol, 1.00 eq) in DCM (10 mL) was added TFA (1.54 g, 13.5 mmol, 1 mL, 8.47 eq) at 20° C. The mixture was stirred at 20° C. for 12 h. LC-MS showed tert-butyl 4-[2-[2-[5-[[5-chloro-4-(3-phenylphenyl)pyrimidin-2-yl]amino]-3-pyridyl]-1-oxo-2,8-diazaspiro[4.5]decan-8-yl]-2-oxo-ethyl]-4-hydroxy-piperidine-1-carboxylate (161-5) remained and one main peak with the desired mass was detected. The mixture was stirred at 20° C. for 2 h. LCMS showed tert-butyl 4-[2-[2-[5-[[5-chloro-4-(3-phenylphenyl)pyrimidin-2-yl]amino]-3-pyridyl]-1-oxo-2,8-diazaspiro[4.5]decan-8-yl]-2-oxo-ethyl]-4-hydroxy-piperidine-1-carboxylate (161-5) was consumed completely and one main peak with the desired mass was detected. The mixture was concentrated under reduced pressure to give the title compound (161-6) (1.00 g, crude, TFA) as a yellow oil. LCMS (ES⁺): m/z 652.2 [M+H]⁺.

Synthesis of 2-[5-[[5-chloro-4-(3-phenylphenyl)pyrimidin-2-yl]amino]-3-pyridyl]-8-[2-[4-hydroxy-1-(4-nitrophenyl)-4-piperidyl]acetyl]-2,8-diazaspiro[4.5]decan-1-one (161-7). To a solution of 2-[5-[[5-chloro-4-(3-phenylphenyl)pyrimidin-2-yl]amino]-3-pyridyl]-8-[2-(4-hydroxy-4-piperidyl)acetyl]-2,8-diazaspiro[4.5]decan-1-one (161-6) (1.00 g, 1.31 mmol, 1.00 eq, TFA) in DMF (10 mL) was added K₂CO₃ (361 mg, 2.61 mmol, 2.00 eq) and 1-fluoro-4-nitro-benzene (184 mg, 1.30 mmol, 138 μL, 9.99e-1 eq) at 20° C. The mixture was stirred at 80° C. for 12 h. LC-MS showed 2-[5-[[5-chloro-4-(3-phenylphenyl)pyrimidin-2-yl]amino]-3-pyridyl]-8-[2-(4-hydroxy-4-piperidyl)acetyl]-2,8-diazaspiro[4.5]decan-1-one (161-6) was remained and no desired mass was detected. The mixture was stirred at 80° C. for 2 h. LCMS showed 2-[5-[[5-chloro-4-(3-phenylphenyl)pyrimidin-2-yl]amino]-3-pyridyl]-8-[2-(4-hydroxy-4-piperidyl)acetyl]-2,8-diazaspiro[4.5]decan-1-one (161-6) was remained and one main peak with the desired mass was detected. The reaction mixture was quenched by addition H₂O (50 ml) at 25° C., and filtered and filter cake was dried in a vacuum to give the title compound (161-7) (500 mg, crude) as a yellow solid. LCMS (ES⁺): m/z 773.2 [M+H]⁺.

Synthesis of 8-[2-[1-(4-aminophenyl)-4-hydroxy-4-piperidyl]acetyl]-2-[5-[[5-chloro-4-(3-phenylphenyl)pyrimidin-2-yl]amino]-3-pyridyl]-2,8-diazaspiro[4.5]decan-1-one (161-8). To a mixture of Raney-Ni (500 mg, 5.84 mmol, 9.03 eq) in EtOH (5 mL) was added 2-[5-[[5-chloro-4-(3-phenylphenyl)pyrimidin-2-yl]amino]-3-pyridyl]-8-[2-[4-hydroxy-1-(4-nitrophenyl)-4-piperidyl]acetyl]-2,8-diazaspiro[4.5]decan-1-one (161-7) (500 mg, 647 μmol, 1.00 eq) at 20° C. The mixture was stirred at 20° C. for 12 h under H₂ (15 Psi). LCMS showed 2-[5-[[5-chloro-4-(3-phenylphenyl)pyrimidin-2-yl]amino]-3-pyridyl]-8-[2-[4-hydroxy-1-(4-nitrophenyl)-4-piperidyl]acetyl]-2,8-diazaspiro[4.5]decan-1-one (161-7) was remained and one main peak with the desired mass was detected. The mixture was filtered and filtrate was concentrated under reduced pressure to give the title compound (161-8) (400 mg, crude) as a green solid. LCMS (ES⁺): m/z 743.3 [M+H]⁺.

Synthesis of 3-[4-[4-[2-[2-[5-[[5-chloro-4-(3-phenylphenyl)pyrimidin-2-yl]amino]-3-pyridyl]-1-oxo-2,8-diazaspiro[4.5]decan-8-yl]-2-oxo-ethyl]-4-hydroxy-1-piperidyl]anilino]piperidine-2,6-dione (Compound 180). To a solution of 8-[2-[1-(4-aminophenyl)-4-hydroxy-4-piperidyl]acetyl]-2-[5-[[5-chloro-4-(3-phenylphenyl)pyrimidin-2-yl]amino]-3-pyridyl]-2,8-diazaspiro[4.5]decan-1-one (161-8) (400 mg, 538 μmol, 1.00 eq) in DMF (4 mL) were added DIPEA (139 mg, 1.08 mmol, 187 μL, 2.00 eq) and 3-bromopiperidine-2,6-dione (104 mg, 542 μmol, 1.01 eq) at 20° C. The mixture was stirred at 100° C. for 12 h. LCMS showed 8-[2-[1-(4-aminophenyl)-4-hydroxy-4-piperidyl]acetyl]-2-[5-[[5-chloro-4-(3-phenylphenyl)pyrimidin-2-yl]amino]-3-pyridyl]-2,8-diazaspiro[4.5]decan-1-one (161-8) was consumed completely and one main peak with the desired mass was detected. The reaction mixture was quenched by addition H₂O (20 ml) at 25° C., and then extracted with EtOAc (10 mL*3). The combined organic layers were washed with brine (20 mL), dried over Na₂SO₄, filtered and concentrated under reduced pressure to give a residue. The residue was purified by prep-HPLC (column: Phenomenex Luna C18 75*30 mm*3 um; mobile phase: [water (FA)-ACN]; B %: 15%-50%, 8 min) to give the title compound (Compound 180) (59.0 mg, 64.6 μmol, 12.0% yield, 93.5% purity) as a gray solid. LCMS (ES⁺): m/z 854.3 [M+H]⁺.

### Example 162. 3-((4-(4-(2-(2-(5-((5-chloro-4-(2-phenylmorpholino)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 181)

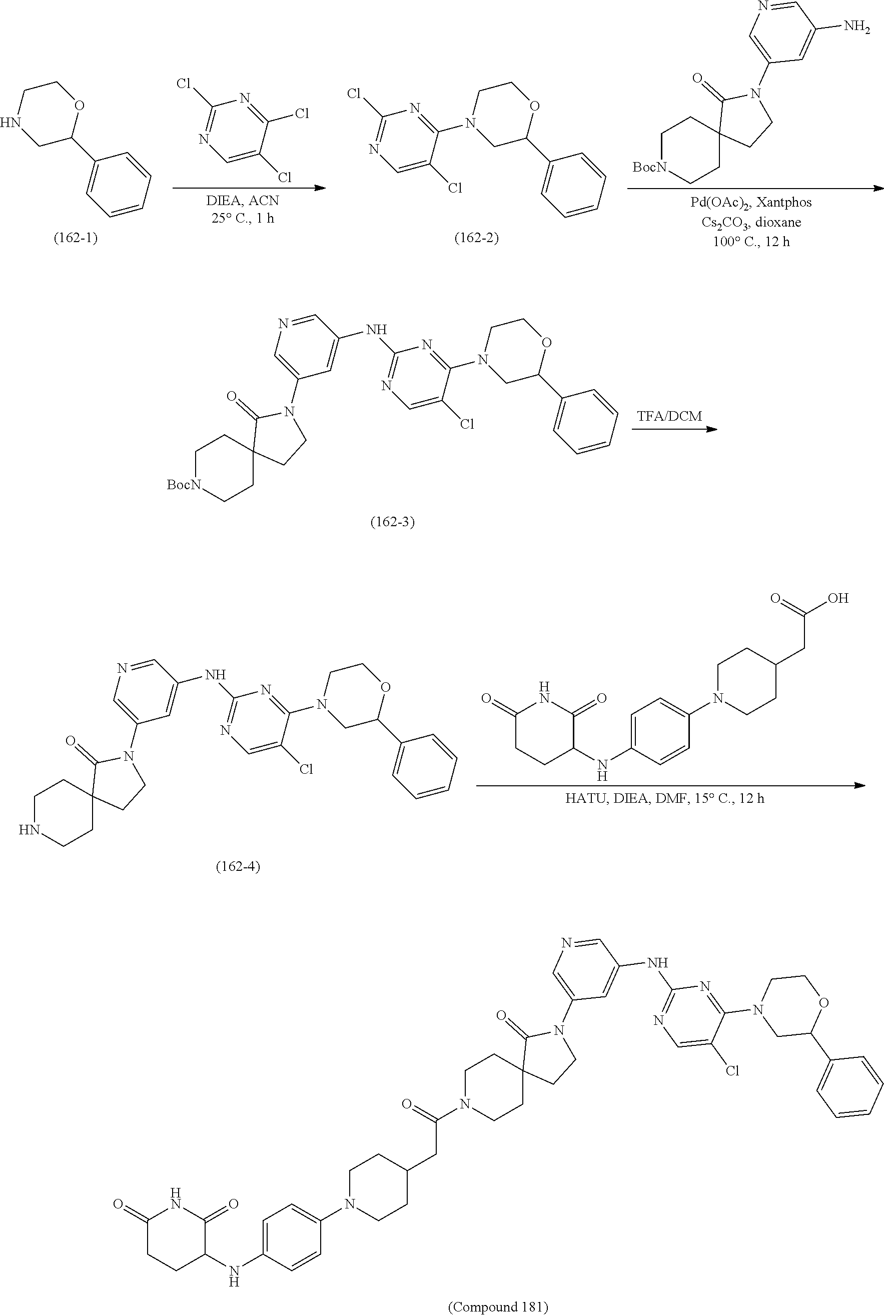

Synthesis of 4-(2,5-dichloropyrimidin-4-yl)-2-phenyl-morpholine (162-2). To a solution of 2-phenylmorpholine (162-1) (9.08 g, 45.4 mmol, 0.817 eq, HCl) in ACN (30 mL) were added DIEA (21.6 g, 167 mmol, 29.1 mL, 3.00 eq) and 2,4,5-trichloropyrimidine (10.2 g, 55.6 mmol, 1.00 eq) at 25° C. The mixture was stirred at 25° C. for 1 h. LCMS showed that 2-phenylmorpholine (162-1) was consumed completely and desired mass was detected. The reaction mixture was filtered and the filtrate concentrated under high vacuum. The residue was purified by column chromatography (SiO₂, Petroleum ether/Ethyl acetate=1/0 to 0/1) to give the title compound (162-2) (12.6 g, crude) as a yellow solid. LCMS (ES⁺): m/z 310.0 [M+H]⁺.

Synthesis of tert-butyl 2-[5-[[5-chloro-4-(2-phenylmorpholin-4-yl)pyrimidin-2-yl]amino]-3-pyridyl]-1-oxo-2,8-diazaspiro[4.5]decane-8-carboxylate (162-3). A mixture of 4-(2,5-dichloropyrimidin-4-yl)-2-phenyl-morpholine (162-2) (11.0 g, 35.5 mmol, 1.00 eq), tert-butyl 2-(5-amino-3-pyridyl)-1-oxo-2,8-diazaspiro[4.5]decane-8-carboxylate (13.5 g, 39.0 mmol, 1.10 eq), Cs₂CO₃ (34.7 g, 106 mmol, 3.00 eq), Pd(OAc)₂ (796 mg, 3.55 mmol, 0.100 eq) and Xantphos (2.05 g, 3.55 mmol, 0.100 eq) in dioxane (100 mL) was degassed and purged with N₂ at 25° C. for 3 times, and then the mixture was stirred at 100° C. for 12 h under N₂ atmosphere. LCMS showed that 4-(2,5-dichloropyrimidin-4-yl)-2-phenyl-morpholine (162-2) was remaining and the desired mass was detected. H₂O 50 mL was added to the mixture, and it was extracted with EtOAc (100 mL*3). The combined organic layers were washed with sat.aq. NaCl (50 mL), dried over Na₂SO₄, filtered and the filtrate was concentrated under reduced pressure to give a residue. The residue was purified by prep-HPLC (Phenomenex luna C18 (250*70 mm, 15 um); mobile phase: [water (FA)-ACN]; B %: 65%-95%, 20 min) to give the title compound (162-3) (7.10 g, 10.8 mmol, 30.6% yield, 94.7% purity) as a white solid. LCMS (ES⁺): m/z 620.2 [M+H]⁺.

Synthesis of 2-(5-((5-chloro-4-(2-phenylmorpholino)pyrimidin-2-yl)amino)pyridin-3-yl)-2,8-diazaspiro[4.5]decan-1-one (162-4). A solution of tert-butyl 2-[5-[[5-chloro-4-(2-phenylmorpholin-4-yl)pyrimidin-2-yl]amino]-3-pyridyl]-1-oxo-2,8-diazaspiro[4.5]decane-8-carboxylate (162-3) (410 mg, 661 μmol, 1.00 eq) in TFA (1 mL) and DCM (3 mL) was stirred at 15° C. for 12 h. LCMS showed that that tert-butyl 2-[5-[[5-chloro-4-(2-phenylmorpholin-4-yl)pyrimidin-2-yl]amino]-3-pyridyl]-1-oxo-2,8-diazaspiro[4.5]decane-8-carboxylate (162-3) was consumed completely and one main peak with desired mass was detected. The mixture was concentrated under reduced pressure to give the title compound (162-4) (930 mg, crude, TFA) as a brown oil. LCMS (ES⁺): m/z 520.2 [M+H]⁺.

Synthesis of 3-((4-(4-(2-(2-(5-((5-chloro-4-(2-phenylmorpholino)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 181). To a solution of 2-[1-[4-[(2,6-dioxo-3-piperidyl)amino]phenyl]-4-piperidyl]acetic acid (150 mg, 434 μmol, 1.00 eq) and 2-[5-[[5-chloro-4-(2-phenylmorpholin-4-yl)pyrimidin-2-yl]amino]-3-pyridyl]-2,8-diazaspiro[4.5]decan-1-one (162-4) (242 mg, 381 μmol, 8.78e-1 eq, TFA) in DMF (2 mL) were added HATU (248 mg, 651 μmol, 1.50 eq) and DIPEA (140 mg, 1.09 mmol, 189 μL, 2.50 eq) at 15° C. The mixture was stirred at 15° C. for 12 h. LCMS showed 2-[1-[4-[(2,6-dioxo-3-piperidyl)amino]phenyl]-4-piperidyl]acetic acid was consumed completely and the desired mass was detected. The mixture was diluted with H₂O (10 mL) and extracted with EtOAc (3 mL*3). The organic layers were washed with brine (15 mL) dried over Na₂SO₄, filtered and concentrated under reduced pressure. The residue was purified by prep-HPLC (column: Phenomenex Luna C18 75*30 mm*3 um; mobile phase: [water (FA)-ACN]; B %: 15%-50%, 8 min) to give the title compound (Compound 181) (23.0 mg, 27.1 μmol, 6.24% yield, 99.8% purity) was obtained as a gray solid. (LCMS (ES⁺): m/z 847.4 [M+H]⁺.

### Example 163. Synthesis of 3-((4-(4-(2-(2-(5-((5-chloro-4-(3-(piperidin-1-yl)phenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 182)

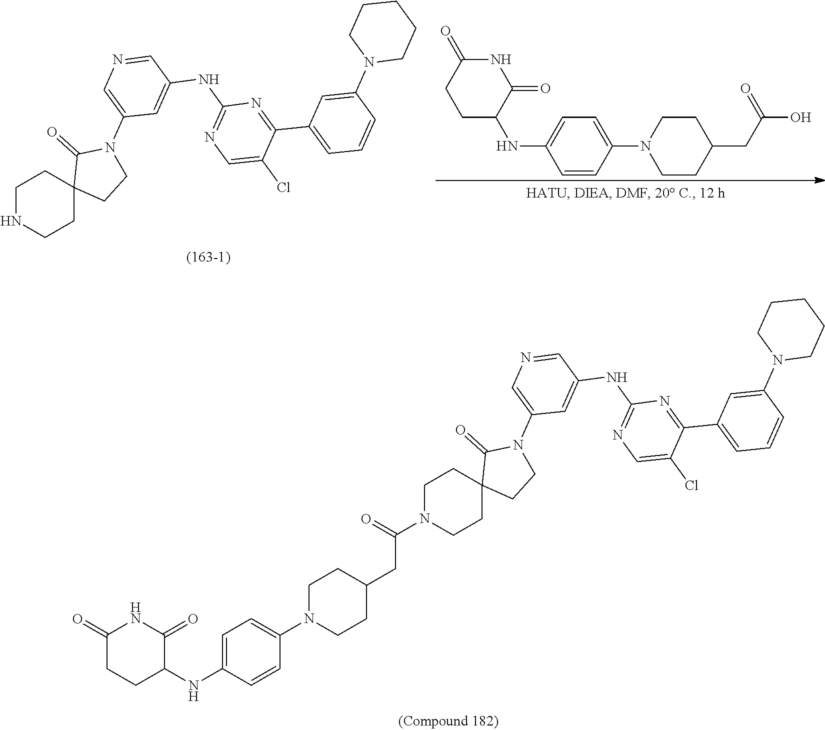

Synthesis of 3-((4-(4-(2-(2-(5-((5-chloro-4-(3-(piperidin-1-yl)phenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 182). To a solution of 2-(1-(4-((2,6-dioxopiperidin-3-yl)amino)phenyl)piperidin-4-yl)acetic acid (133 mg, 386 μmol, 1.00 eq) in DMF (2 mL) were added HATU (176 mg, 463 μmol, 1.20 eq), DIEA (249 mg, 1.93 mmol, 336 μL, 5.00 eq) at 20° C., then 2-(5-((5-chloro-4-(3-(piperidin-1-yl)phenyl)pyrimidin-2-yl)amino)pyridin-3-yl)-2,8-diazaspiro[4.5]decan-1-one (163-1) (200 mg, 386 μmol, 1.00 eq) was added to the mixture, the mixture was stirred at 20° C. for 2 h. LCMS showed that 2-(5-((5-chloro-4-(3-(piperidin-1-yl)phenyl)pyrimidin-2-yl)amino)pyridin-3-yl)-2,8-diazaspiro[4.5]decan-1-one (163-1) was consumed and the desired mass was detected. To the mixture was added H₂O (5 mL) and the mixture was extracted with EtOAc (3 mL*3). The combined organic layers were washed with saturated aqueous NaCl solution (5 mL*3), dried over Na₂SO₄, filtered and the filtrate was concentrated in vacuo. This mixture was purified by acidic prep-HPLC (column: Phenomenex luna C18 100*40 mm*3 um; mobile phase: [water (FA)-ACN]; B %: 15%-45%, 8 min) give the title compound (Compound 182) (7.50 mg, 8.77 μmol, 2.27% yield, 98.9% purity) as brown solid. LCMS (ES⁺): m/z 845.4 [M+H]⁺.

### Example 164. Synthesis of N-(5-((4-([1,1′-biphenyl]-3-yl)-5-chloropyrimidin-2-yl)amino)pyridin-3-yl)-3-(4-(1-(4-((2,6-dioxopiperidin-3-yl)amino)phenyl)piperidin-4-yl)piperazin-1-yl)propanamide (Compound 183)

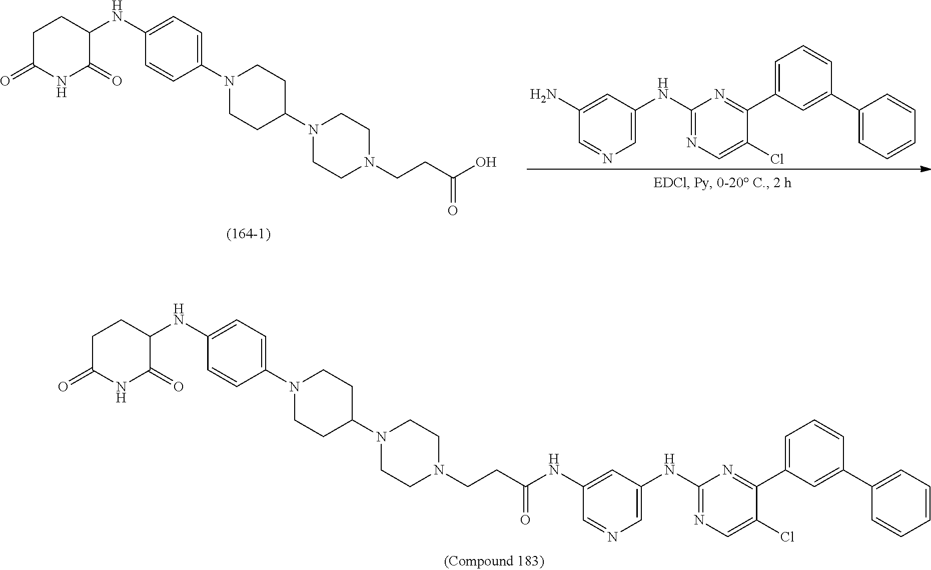 [SMILES: O=C(O)CCN1CCN(C2CCN(c3ccc(NC4CCC(=O)NC4=O)cc3)CC2)CC1 ; Nc1cncc(Nc2ncc(Cl)c(-c3cccc(-c4ccccc4)c3)n2)c1 ; O=C1CCC(Nc2ccc(N3CCC(N4CCN(CCC(=O)Nc5cncc(Nc6ncc(Cl)c(-c7cccc(-c8ccccc8)c7)n6)c5)CC4)CC3)cc2)C(=O)N1]

Synthesis of N-(5-((4-([1,1′-biphenyl]-3-yl)-5-chloropyrimidin-2-yl)amino)pyridin-3-yl)-3-(4-(1-(4-((2,6-dioxopiperidin-3-yl)amino)phenyl)piperidin-4-yl)piperazin-1-yl)propanamide (Compound 183). To a solution of 3-(4-(1-(4-((2,6-dioxopiperidin-3-yl)amino)phenyl)piperidin-4-yl)piperazin-1-yl)propanoic acid (164-1) (100 mg, 225 umol, 1.00 eq) and N3-(4-([1,1′-biphenyl]-3-yl)-5-chloropyrimidin-2-yl)pyridine-3,5-diamine (42.1 mg, 113 umol, 0.500 eq) in Py (1.5 mL) at 20° C., then EDCI (64.8 mg, 338 umol, 1.50 eq) was added to the mixture at 0° C. The mixture was stirred at 20° C. for 2 h. LCMS showed 3-(4-(1-(4-((2,6-dioxopiperidin-3-yl)amino)phenyl)piperidin-4-yl)piperazin-1-yl)propanoic acid (164-1) was consumed and the desired mass was detected. The mixture was concentrated in vacuo. This mixture was purified by acidic prep-HPLC (column: C18-6 100*30 mm*5 um; mobile phase: [water (FA)-ACN]; B %: 15%-50%, 8 min) to give the title compound (Compound 183) (14.4 mg, 17.3 umol, 9.65% yield, 96.1% purity) as a gray solid. LCMS (ES⁺): m/z 799.3 [M+H]⁺.

### Example 165. Synthesis of 3-((4-(4-(2-(2-(5-((5-chloro-4-(3-(pyrrolidin-1-yl)phenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 184)

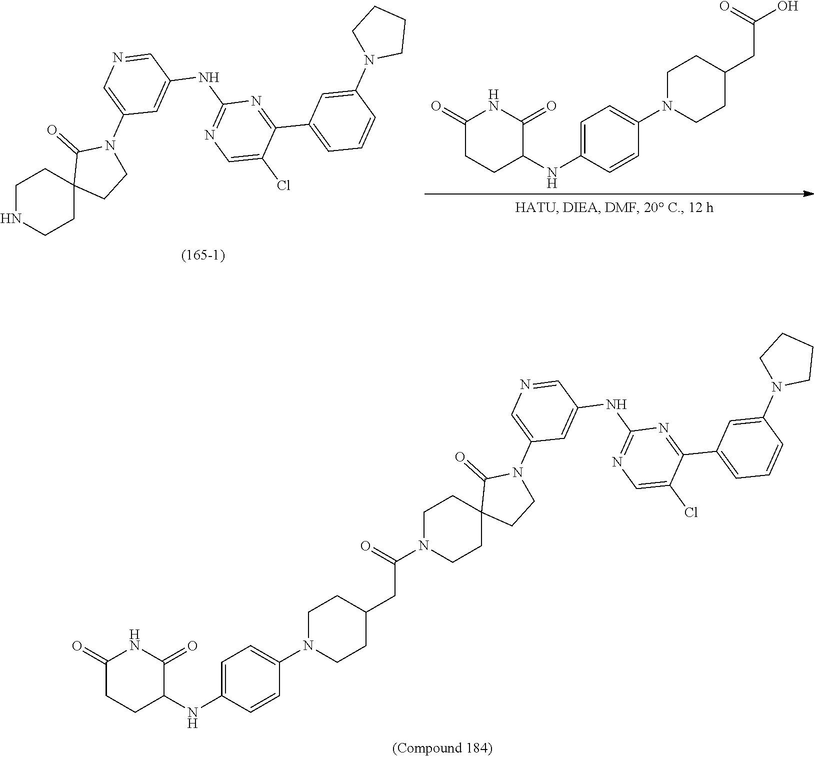 [SMILES: O=C1N(c2cncc(Nc3ncc(Cl)c(-c4cccc(N5CCCC5)c4)n3)c2)CCC12CCNCC2 ; O=C(O)CC1CCN(c2ccc(NC3CCC(=O)NC3=O)cc2)CC1 ; O=C1CCC(Nc2ccc(N3CCC(CC(=O)N4CCC5(CC4)CCN(c4cncc(Nc6ncc(Cl)c(-c7cccc(N8CCCC8)c7)n6)c4)C5=O)CC3)cc2)C(=O)N1]

Synthesis of 3-((4-(4-(2-(2-(5-((5-chloro-4-(3-(pyrrolidin-1-yl)phenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 184). To a solution of 2-[5-[[5-chloro-4-(3-pyrrolidin-1-ylphenyl)pyrimidin-2-yl]amino]-3-pyridyl]-2,8-diazaspiro[4.5]decan-1-one (165-1) (130 mg, 257 μmol, 1.00 eq) in DMF (3 mL) were added HATU (147 mg, 386 μmol, 1.50 eq), 2-[1-[4-[(2,6-dioxo-3-piperidyl)amino]phenyl]-4-piperidyl]acetic acid (106 mg, 309 μmol, 1.20 eq) and DIEA (166 mg, 1.29 mmol, 224 μL, 5.00 eq) at 20° C. The mixture was stirred at 20° C. for 12 h. LCMS showed 2-[5-[[5-chloro-4-(3-pyrrolidin-1-ylphenyl)pyrimidin-2-yl]amino]-3-pyridyl]-2,8-diazaspiro[4.5]decan-1-one (165-1) was consumed completely and the desired mass was detected. The reaction mixture was filtered and the filtrate concentrated under reduced pressure. The residue was purified by prep-HPLC (Phenomenex Luna C18 200*40 mm*10 um; mobile phase: [water (FA)-ACN]; B %: 15%-45%, 8 min) to give the title compound (Compound 184) (10.1 mg, 11.9 μmol, 4.65% yield, 99.8% purity) as a gray solid. LCMS (ES⁺): m/z 831.3 [M+H]⁺.

### Example 166. Synthesis of (3-[4-[l-[2-[2-[5-[[5-chloro-4-[3-(1-piperidyl)phenyl]pyrimidin-2-yl]amino]-3-pyridyl]-1-oxo-2,8-diazaspiro[4.5]decan-8-yl]-2-oxo-ethyl]-4-piperidyl]anilino]piperidine-2,6-dione (Compound 185)

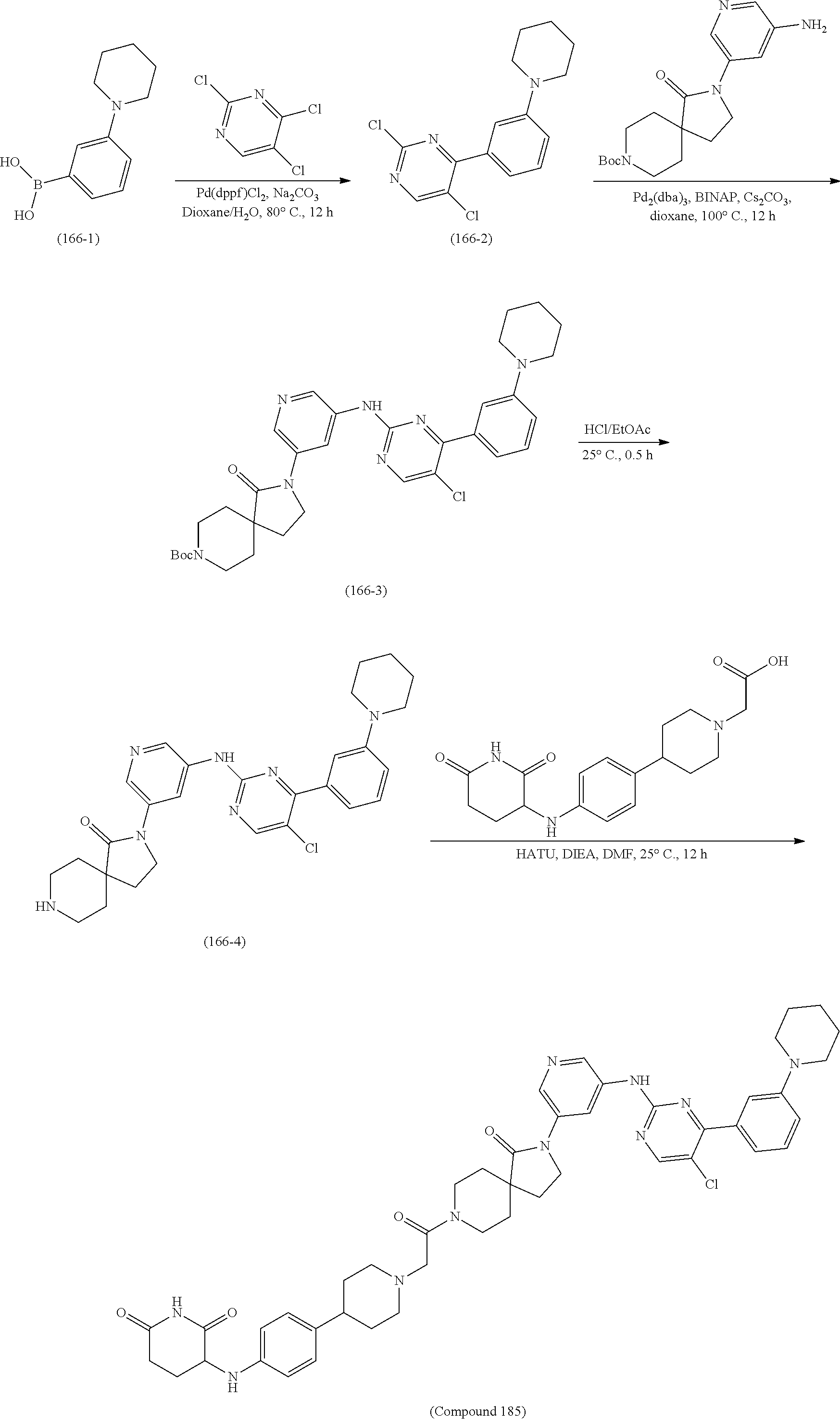

Synthesis of 2,5-dichloro-4-[3-(1-piperidyl)phenyl]pyrimidine (166-2) To a solution of [3-(1-piperidyl)phenyl]boronic acid (166-1) (3.00 g, 14.6 mmol, 1.00 eq) and 2,4,5-trichloropyrimidine (2.95 g, 16.1 mmol, 1.10 eq) in Dioxane (10 mL) and H₂O (2 mL) were added Na₂CO₃ (4.65 g, 43.8 mmol, 3.00 eq) and Pd(dppf)Cl₂ (2.14 g, 2.93 mmol, 0.200 eq) at 20° C. under N₂. The mixture was stirred at 80° C. for 12 h. LCMS showed the desired mass was detected. H₂O 20 mL was added to the mixture, and extracted with DCM (20 mL*3). The combined organic layers were dried over Na₂SO₄, filtered and concentrated under reduced pressure. The residue was purified by column chromatography (SiO₂, Petroleum ether/Ethyl acetate=1/1) to give the title compound (166-2) (2.30 g, 7.46 mmol, 51.0% yield) as a yellow solid. LCMS (ES⁺): m/z 308.0 [M+H]⁺.

Synthesis of tert-butyl 2-(5-((5-chloro-4-(3-(piperidin-1-yl)phenyl)pyrimidin-2-yl)amino)pyridin-3-yl)-1-oxo-2,8-diazaspiro[4.5]decane-8-carboxylate (166-3). To a solution of 2,5-dichloro-4-(3-(piperidin-1-yl)phenyl)pyrimidine (166-2) (1.00 g, 3.24 mmol, 1.00 eq), tert-butyl 2-(5-amino-3-pyridyl)-1-oxo-2,8-diazaspiro[4.5]decane-8-carboxylate (1.24 g, 3.57 mmol, 1.10 eq), dicesium; carbonate (2.64 g, 8.11 mmol, 2.50 eq) and BINAP (202 mg, 324 μmol, 0.100 eq) in dioxane (20 mL) was added the Pd₂(dba)₃ (297 mg, 324 μmol, 0.100 eq) at 25° C. under N₂. The mixture was stirred at 100° C. for 12 h under N₂. LCMS showed that the desired mass was detected. H₂O 100 mL was added to the mixture, and extracted with EtOAc (100 mL*3). The combined organic layers were dried over Na₂SO₄, filtered and the filtrate was concentrated under reduced pressure. The residue was purified by column chromatography (SiO₂, Petroleum ether/Ethyl acetate=3/1) to give the title compound (166-3) (1.30 g, crude) as a yellow solid. LCMS (ES⁺): m/z 618.5 [M+H]⁺.

Synthesis of 2-[5-[[5-chloro-4-[3-(1-piperidyl)phenyl]pyrimidin-2-yl]amino]-3-pyridyl]-2,8-diazaspiro[4.5]decan-1-one (166-4). A solution of tert-butyl 2-[5-[[5-chloro-4-[3-(1-piperidyl)phenyl]pyrimidin-2-yl]amino]-3-pyridyl]-1-oxo-2,8-diazaspiro[4.5]decane-8-carboxylate (166-3) (220 mg, 355 μmol, 1.00 eq) in HCl/EtOAc (4 M, 1 mL) was stirred at 25° C. for 0.5 h. LCMS showed the desired mass was detected. The reaction mixture was concentrated under reduced pressure to give the title compound (166-4) (180 mg, crude) as a yellow solid. LCMS (ES⁺): m/z 518.3 [M+H]⁺.

Synthesis of 3-[4-[1-[2-[2-[5-[[5-chloro-4-[3-(1-piperidyl)phenyl]pyrimidin-2-yl]amino]-3-pyridyl]-1-oxo-2,8-diazaspiro[4.5]decan-8-yl]-2-oxo-ethyl]-4-piperidyl]anilino]piperidine-2,6-dione (Compound 185). To a solution of 2-[5-[[5-chloro-4-[3-(1-piperidyl)phenyl]pyrimidin-2-yl]amino]-3-pyridyl]-2,8-diazaspiro[4.5]decan-1-one (166-4) (130 mg, 250 μmol, 1.00 eq) and 2-[4-[4-[(2,6-dioxo-3-piperidyl)amino]phenyl]-1-piperidyl]acetic acid (95.3 mg, 276 μmol, 1.10 eq) in DMF (2 mL) were added HATU (143 mg, 376 μmol, 1.50 eq) and DIEA (162 mg, 1.25 mmol, 218 μL, 5.00 eq) at 25° C. The mixture was stirred at 25° C. for 12 h. LCMS showed the desired mass was detected. H₂O 10 mL was added to the mixture, and extracted with EtOAc (10 mL*3). The combined organic layers were dried over Na₂SO₄, filtered and the filtrate was concentrated under reduced pressure. The residue was purified by prep-HPLC (column: Phenomenex luna C18 80*40 mm*3 um; mobile phase: [water (FA)-ACN]; B %: 1%-45%, 8 min) to give the title compound (Compound 185) (41.1 mg, 47.1 μmol, 18.8% yield, 97.5% purity, 0.1 FA) as a yellow solid LCMS (ES⁺): m/z 845.5[M+H]⁺.

### Example 167 Synthesis of 3-[4-[1-[2-[2-[5-[[5-chloro-4-(2-phenylmorpholin-4-yl)pyrimidin-2-yl]amino]-3-pyridyl]-1-oxo-2,8-diazaspiro[4.5]decan-8-yl]-2-oxo-ethyl]-4-piperidyl]anilino]piperidine-2,6-dione (Compound 186)

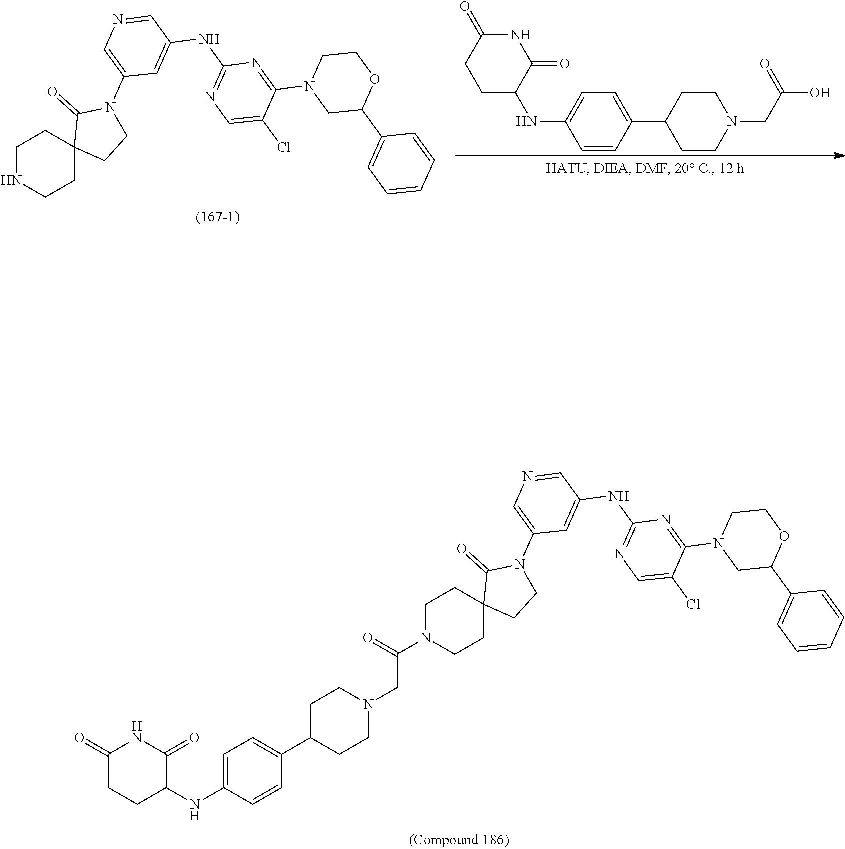 [SMILES: O=C1N(c2cncc(Nc3ncc(Cl)c(N4CCOC(c5ccccc5)C4)n3)c2)CCC12CCNCC2 ; O=C(O)CN1CCC(c2ccc(NC3CCC(=O)NC3=O)cc2)CC1 ; O=C1CCC(Nc2ccc(C3CCN(CC(=O)N4CCC5(CC4)CCN(c4cncc(Nc6ncc(Cl)c(N7CCOC(c8ccccc8)C7)n6)c4)C5=O)CC3)cc2)C(=O)N1]

Synthesis of 3-[4-[1-[2-[2-[5-[[5-chloro-4-(2-phenylmorpholin-4-yl)pyrimidin-2-yl]amino]-3-pyridyl]-1-oxo-2,8-diazaspiro[4.5]decan-8-yl]-2-oxo-ethyl]-4-piperidyl]anilino]piperidine-2,6-dione (Compound 186). To a solution of 2-[4-[4-[(2,6-dioxo-3-piperidyl)amino]phenyl]-1-piperidyl]acetic acid (150 mg, 326.50 μmol, 1.00 eq, TFA) in THF (5 mL) were added 2-[5-[[5-chloro-4-(2-phenylmorpholin-4-yl)pyrimidin-2-yl]amino]-3-pyridyl]-2,8-diazaspiro[4.5]decan-1-one (167-1) (170 mg, 268 μmol, 8.21e-1 eq, TFA) and HATU (373 mg, 981 μmol, 3.00 eq), DIPEA (211 mg, 1.63 mmol, 284 μL, 5.00 eq) at 20° C. The mixture was stirred at 20° C. for 12 h. LCMS showed that 2-(5-((5-chloro-4-(2-phenylmorpholino)pyrimidin-2-yl)amino)pyridin-3-yl)-2,8-diazaspiro[4.5]decan-1-one (167-1) was consumed completely and one main peak with the desired mass was detected. The mixture was filtered and the filtrate was concentrated under reduced pressure. The residue was purified by prep-HPLC (column: Phenomenex Luna C18 75*30 mm*3 um; mobile phase: [water (FA)-ACN]; B %: 30%-65%, 8 min) to give the title compound (Compound 186) (9.70 mg, 11.5 μmol, 2.64% yield, 100% purity) as a white solid. LCMS (ES⁺): m/z 847.4 [M+H]⁺.

### Example 168. Synthesis of N-(5-((4-([1,1′-biphenyl]-3-yl)-5-chloropyrimidin-2-yl)amino)pyridin-3-yl)-1-((1-(4-((2,6-dioxopiperidin-3-yl)amino)phenyl)piperidin-4-yl)methyl)piperidine-4-carboxamide (Compound 187)

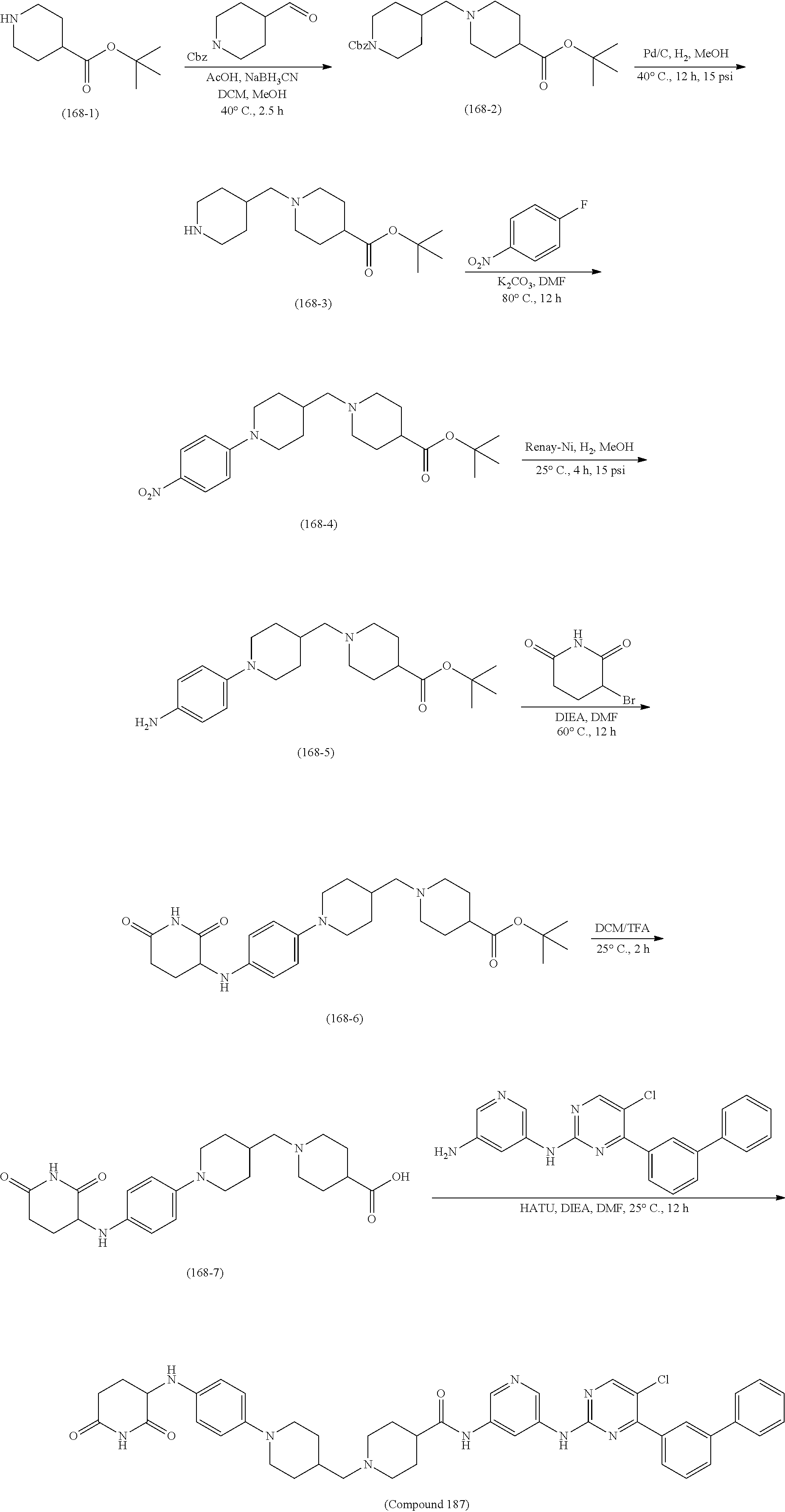

Synthesis of benzyl 4-((4-(tert-butoxycarbonyl)piperidin-1-yl)methyl)piperidine-1-carboxylate (168-2). To a solution of tert-butyl piperidine-4-carboxylate (168-1) (2.00 g, 9.02 mmol, 1.00 eq) in DCM (10 mL) and MeOH (10 mL) was added AcOH (54.2 mg, 902 umol, 51.6 uL, 0.100 eq) and benzyl 4-formylpiperidine-1-carboxylate (4.46 g, 18.0 mmol, 2.00 eq) at 25° C., the mixture was stirred at 40° C. for 0.5 hr. Then NaBH₃CN (1.13 g, 18.0 mmol, 2.00 eq) was added to the mixture at 40° C., the mixture was stirred at 40° C. for 2 hr. LCMS showed tert-butyl piperidine-4-carboxylate (168-1) was consumed and the desired mass was detected. The mixture was concentrated in vacuo. The mixture was purified by MPLC (SiO₂, Ethyl acetate/Methanol=1/0 to 10/1) to give the title compound (168-2) (3.00 g, 7.20 mmol, 79.8% yield) as yellow oil liquid. LCMS (ES⁺): m/z 417.3 [M+H]⁺.

Synthesis of tert-butyl 1-(piperidin-4-ylmethyl)piperidine-4-carboxylate (168-3). To a stirred solution of benzyl 4-((4-(tert-butoxycarbonyl)piperidin-1-yl)methyl)piperidine-1-carboxylate (168-2) (1.30 g, 3.12 mmol, 1.00 eq) in MeOH (25.0 mL) was added 10% Pd/C (1.30 g, 50% purity) at 25° C. The mixture was stirred at 40° C. for 12 h under H₂ (15 Psi). LCMS showed benzyl 4-((4-(tert-butoxycarbonyl)piperidin-1-yl)methyl)piperidine-1-carboxylate (168-2) was consumed completely and the desired mass was detected. The mixture was filtered, the filtrate was concentrate in vacuo to give the title compound (168-3) (870 mg, crude) as a white solid. LCMS (ES⁺): m/z 283.2 [M+H]⁺.

Synthesis of tert-butyl 1-((1-(4-nitrophenyl)piperidin-4-yl)methyl)piperidine-4-carboxylate (168-4). To solution of tert-butyl 1-(piperidin-4-ylmethyl)piperidine-4-carboxylate (168-3) (870 mg, 3.08 mmol, 1.00 eq) and 1-fluoro-4-nitro-benzene (434.7 mg, 3.08 mmol, 327 μL, 1.00 eq) in DMF (15.0 mL) was added K₂CO₃ (1.28 g, 9.24 mmol, 3.00 eq) at 25° C. The mixture was stirred at 80° C. for 12 h. LCMS showed tert-butyl 1-(piperidin-4-ylmethyl)piperidine-4-carboxylate (168-3) was consumed completely and the desired mass was detected. H₂O (30 mL) was added to the solution and the mixture was filtered, the filter cake was dried in vacuo to give the title compound (168-4) (740 mg, crude) as a yellow solid. LCMS (ES⁺): m/z 404.3 [M+H]⁺.

Synthesis of tert-butyl 1-((1-(4-aminophenyl)piperidin-4-yl)methyl)piperidine-4-carboxylate (168-5). To a stirred solution of tert-butyl 1-((1-(4-nitrophenyl)piperidin-4-yl)methyl)piperidine-4-carboxylate (168-4) (700 mg, 1.73 mmol, 1.00 eq) in MeOH (5.00 mL) was added Raney-Ni (900 mg) at 25° C. The mixture was stirred at 25° C. for 2 h under H₂ (15 Psi). LCMS showed tert-butyl 1-((1-(4-nitrophenyl)piperidin-4-yl)methyl)piperidine-4-carboxylate (168-4) remained and the desired mass was detected. Then the mixture was stirred at 25° C. for 2 h under H₂ (15 PSI). LCMS showed tert-butyl 1-((1-(4-nitrophenyl)piperidin-4-yl)methyl)piperidine-4-carboxylate (168-4) was consumed completely and the desired mass was detected. The mixture was filtered, the filtrate was concentrate in vacuo to give the title compound (168-5) (724 mg, crude) as a black solid. LCMS (ES⁺): m/z 374.3 [M+H]⁺.

Synthesis of tert-butyl 1-((1-(4-((2,6-dioxopiperidin-3-yl)amino)phenyl)piperidin-4-yl)methyl)piperidine-4-carboxylate (168-6). To solution of tert-butyl 1-((1-(4-aminophenyl)piperidin-4-yl)methyl)piperidine-4-carboxylate (168-5) (724 mg, 1.94 mmol, 1.00 eq) and 3-bromopiperidine-2,6-dione (930 mg, 4.85 mmol, 2.50 eq) in DMF (10 mL) was added DIEA (752 mg, 5.81 mmol, 1.01 mL, 3.00 eq) at 25° C., then the mixture was stirred at 60° C. for 2 h. LCMS showed tert-butyl 1-((1-(4-aminophenyl)piperidin-4-yl)methyl)piperidine-4-carboxylate (168-5) remained and the desired mass was detected. Then the mixture was stirred at 60° C. for 12 h. LCMS showed tert-butyl 1-((1-(4-aminophenyl)piperidin-4-yl)methyl)piperidine-4-carboxylate (168-5) was consumed completely and the desired mass was detected. The mixture was filtered and the filter cake was dried in vacuo to give the title compound (168-6) (650 mg, crude) as a black solid. LCMS (ES⁺): m/z 485.4 [M+H]⁺.

Synthesis of 1-((1-(4-((2,6-dioxopiperidin-3-yl)amino)phenyl)piperidin-4-yl)methyl)piperidine-4-carboxylic acid (168-7). To a solution of tert-butyl 1-((1-(4-((2,6-dioxopiperidin-3-yl)amino)phenyl)piperidin-4-yl)methyl)piperidine-4-carboxylate (168-6) (200 mg, 413 μmol, 1.00 eq) in DCM (1.50 mL) and TFA (0.50 mL) 25° C. The mixture was stirred at ° C. for 1 h. LCMS showed tert-butyl 1-((1-(4-((2,6-dioxopiperidin-3-yl)amino)phenyl)piperidin-4-yl)methyl)piperidine-4-carboxylate (168-6) remained and the desired mass was detected. Then the mixture was stirred at 25° C. for 1 h. LCMS showed tert-butyl 1-((1-(4-((2,6-dioxopiperidin-3-yl)amino)phenyl)piperidin-4-yl)methyl)piperidine-4-carboxylate (168-6) was consumed completely and the desired mass was detected. The mixture was concentrated in vacuo to give the title compound (168-7) (200 mg, crude) as a yellow oil. LCMS (ES⁺): m/z 429.2 [M+H]⁺.

Synthesis of N-(5-((4-([1,1′-biphenyl]-3-yl)-5-chloropyrimidin-2-yl) amino) pyridin-3-yl)-1-((1-(4-((2, 6-dioxopiperidin-3-yl) amino) phenyl) piperidin-4-yl) methyl) piperidine-4-carboxamide (Compound 187). To a solution of 1-((1-(4-((2,6-dioxopiperidin-3-yl)amino)phenyl)piperidin-4-yl)methyl)piperidine-4-carboxylic acid (168-7) (110 mg, 257 μmol, 1.00 eq) in DMF (6.00 mL) were added HATU (146 mg, 385 μmol, 1.50 eq), DIEA (99.5 mg, 770 μmol, 134 μL, 3.00 eq) at 25° C., and then N3-(4-([1,1′-biphenyl]-3-yl)-5-chloropyrimidin-2-yl)pyridine-3,5-diamine (76.8 mg, 205 μmol, 0.800 eq) was added to the mixture at 25° C. The mixture was stirred at 25° C. for 12 h. LCMS showed 1-((1-(4-((2,6-dioxopiperidin-3-yl)amino)phenyl)piperidin-4-yl)methyl)piperidine-4-carboxylic acid (168-7) was consumed completely and the desired mass was detected. H₂O (10 mL) was added to the solution, the mixture was extracted with EtOAc (10 mL*3), then the combined organic layers were washed with brine (10 mL*3), dried over Na₂SO₄, then concentrated in vacuo. The residue was purified by prep-HPLC (column: Phenomenex Luna C18 200 * 40 mm*10 um; mobile phase: [water (FA)-ACN]; gradient: 15%-55% B over 8 min) to give the title compound (Compound 187) (39.9 mg, 49.8 μmol, 24.6% yield, 97.9% purity) as a black solid. LCMS (ES⁺): m/z 784.2 [M+H]⁺.

### Example 169. Synthesis of 3-((4-(1-(2-(2-(5-((4-(3-(azetidin-1-yl)phenyl)-5-chloropyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 188)

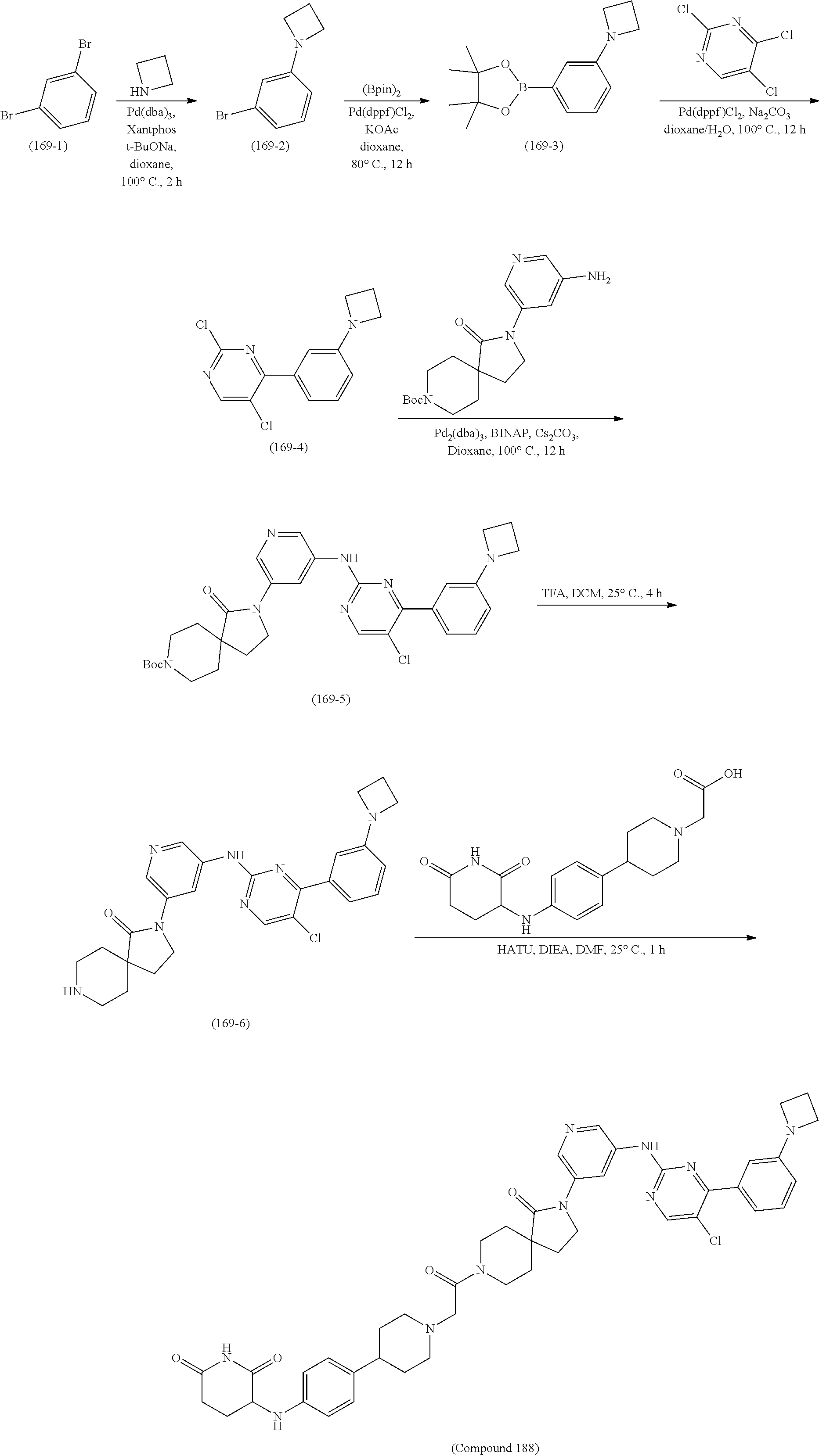

Synthesis of 1-(3-bromophenyl)azetidine (169-2). To a solution of 1,3-dibromobenzene (169-1) (10.0 g, 42.3 mmol, 5.10 mL, 1.00 eq) and azetidine (2.66 g, 46.6 mmol, 3.15 mL, 1.10 eq) in dioxane (100 mL) was added Pd₂(dba)₃ (3.88 g, 4.24 mmol, 0.100 eq) and t-BuONa (12.2 g, 127 mmol, 3.00 eq) and Xantphos (2.45 g, 4.24 mmol, 0.100 eq) at 25° C. The mixture was stirred at 100° C. for 2 h. LC-MS showed the desired mass was detected. H₂O (100 mL) was added to the mixture and the aqueous was extracted with Ethyl acetate (30 mL*3), the combined organic layers were dried over Na₂SO₄ and the mixture was filtered, and concentrated under reduced pressure. The residue was purified by column chromatography (SiO₂, Petroleum ether:Ethyl acetate=1:0 to 1:1) to give the title compound (169-2) (2.38 g, crude) as a white solid. LCMS (ES⁺): m/z 212.2 [M+H]⁺.

Synthesis of 1-[3-(4,4,5,5-tetramethyl-1,3,2-dioxaborolan-2-yl)phenyl]azetidine (169-3). To a solution of 1-(3-bromophenyl)azetidine (169-2) (1.10 g, 5.19 mmol, 1.00 eq) and 4,4,5,5-tetramethyl-2-(4,4,5,5-tetramethyl-1,3,2-dioxaborolan-2-yl)-1,3,2-dioxaborolane (1.58 g, 6.22 mmol, 1.20 eq) in dioxane (40 mL) was added KOAc (1.53 g, 15.5 mmol, 3.00 eq) and Pd(dppf)Cl₂ (379 mg, 518 umol, 0.100 eq) at 25° C. The mixture was stirred at 80° C. for 12 h under N₂. LC-MS showed the desired mass was detected. H₂O (100 mL) was added to the mixture and the aqueous was extracted with Ethyl acetate (30 mL*3), the combined organic layers were dried over Na₂SO₄ and the mixture was filtered, and concentrated under reduced pressure. The residue was purified by column chromatography (SiO₂, Petroleum ether:Ethyl acetate=1:0 to 1:1) to give the title compound (169-3) (2.69 g, crude) as a yellow solid. LCMS (ES⁺): m/z 260.2 [M+H]⁺.

Synthesis of 4-[3-(azetidin-1-yl)phenyl]-2,5-dichloro-pyrimidine (169-4). A mixture of 1-[3-(4,4,5,5-tetramethyl-1,3,2-dioxaborolan-2-yl)phenyl]azetidine (169-3) (2.39 g, 9.22 mmol, 1.00 eq), 2,4,5-trichloropyrimidine (2.03 g, 11.0 mmol, 1.20 eq), Pd(dppf)Cl₂ (674 mg, 922 μmol, 0.100 eq), Na₂CO₃ (1.95 g, 18.4 mmol, 2.00 eq) in dioxane (30 mL) and H₂O (10 mL) was degassed and purged with N₂ for 3 times at 25° C., and then the mixture was stirred at 100° C. for 12 h under N₂ atmosphere. LC-MS showed the desired mass was detected. The reaction mixture was diluted with H₂O 20 mL and extracted with Ethyl acetate (30 mL*3). The combined organic layers were washed with brine 10 mL, dried over Na₂SO₄, filtered and concentrated under reduced pressure. The residue was purified by column chromatography (SiO₂, Dichloromethane:Methanol=1:0 to 10:1) to give the title compound (169-4) (1.20 g, 2.36 mmol, 25.5% yield, 55.0% purity) as a yellow solid. LCMS (ES⁺): m/z 280.0 [M+H]⁺.

Synthesis of tert-butyl 2-[5-[[4-[3-(azetidin-1-yl)phenyl]-5-chloro-pyrimidin-2-yl]amino]-3-pyridyl]-1-oxo-2,8-diazaspiro[4.5]decane-8-carboxylate (169-5). A mixture of 4-[3-(azetidin-1-yl)phenyl]-2,5-dichloro-pyrimidine (169-4) (1.10 g, 3.93 mmol, 1.00 eq), tert-butyl 2-(5-amino-3-pyridyl)-1-oxo-2,8-diazaspiro[4.5]decane-8-carboxylate (1.63 g, 4.71 mmol, 1.20 eq), Pd₂(dba)₃ (359 mg, 392 μmol, 0.100 eq), BINAP (244 mg, 392 μmol, 0.100 eq) and Cs₂CO₃ (3.84 g, 11.7 mmol, 3.00 eq) in dioxane (20 mL) was degassed and purged with N₂ for 3 times at 25° C., and then the mixture was stirred at 100° C. for 12 h under N₂ atmosphere. LC-MS showed the desired mass was detected. The reaction mixture was diluted with H₂O 15 mL and extracted with Ethyl acetate (20 mL*3). The combined organic layers were washed with brine 10 mL, dried over Na₂SO₄, filtered and concentrated under reduced pressure. The residue was purified by column chromatography (SiO₂, Dichloromethane:Methanol=1:0 to 20:1) to give the title compound (169-5) (1.70 g, 1.86 mmol, 47.3% yield, 64.5% purity) as a brown solid. LCMS (ES⁺): m/z 590.3 [M+H]⁺.

Synthesis of 2-[5-[[4-[3-(azetidin-1-yl)phenyl]-5-chloro-pyrimidin-2-yl]amino]-3-pyridyl]-2,8-diazaspiro[4.5]decan-1-one (169-6). A solution of tert-butyl 2-[5-[[4-[3-(azetidin-1-yl)phenyl]-5-chloro-pyrimidin-2-yl]amino]-3-pyridyl]-1-oxo-2,8-diazaspiro[4.5]decane-8-carboxylate (169-5) (1.60 g, 2.71 mmol, 1.00 eq) in DCM (15 mL) was added TFA (4.61 g, 39.9 mmol, 3.00 mL, 99.0% purity, 14.7 eq) was stirred at 25° C. for 4 h. LC-MS showed the desired mass was detected. The reaction mixture was concentrated under reduced pressure to give the title compound (169-6) (1.30 g, TFA, crude) as a black oil. LCMS (ES⁺): m/z 490.2 [M+H]⁺.

Synthesis of 3-((4-(1-(2-(2-(5-((4-(3-(azetidin-1-yl)phenyl)-5-chloropyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 188). To a solution of 2-(5-((4-(3-(azetidin-1-yl)phenyl)-5-chloropyrimidin-2-yl)amino)pyridin-3-yl)-2,8-diazaspiro[4.5]decan-1-one (169-6) (100 mg, 204 μmol, 1.00 eq) in DMF (2 mL) was added DIEA until pH ˜7. To a solution of 2-[4-[4-[(2,6-dioxo-3-piperidyl)amino]phenyl]-1-piperidyl]acetic acid (77.5 mg, 224 μmol, 1.10 eq) in DMF (1 mL) was added DIEA until pH ˜-7. Then HATU (93.1 mg, 244 μmol, 1.20 eq) was added to the mixture of above two solutions at 25° C. The mixture was stirred at 25° C. for 1 h. LC-MS showed the desired mass was detected. The residue was purified by prep-HPLC (column: Phenomenex Luna C18 200*40 mm*10 um; mobile phase: [water (FA)-ACN]; B %: 15%-50%, 8 min) to give the title compound (Compound 188) (26.5 mg, 31.0 μmol, 15.2% yield, 98.0% purity, 0.4 FA) as a yellow solid. LCMS (ES⁺): m/z 817.3 [M+H]⁺.

### Example 170. Synthesis of 3-((4-(4-(2-(2-(5-((5-chloro-4-(3-cyclohexylphenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 189)

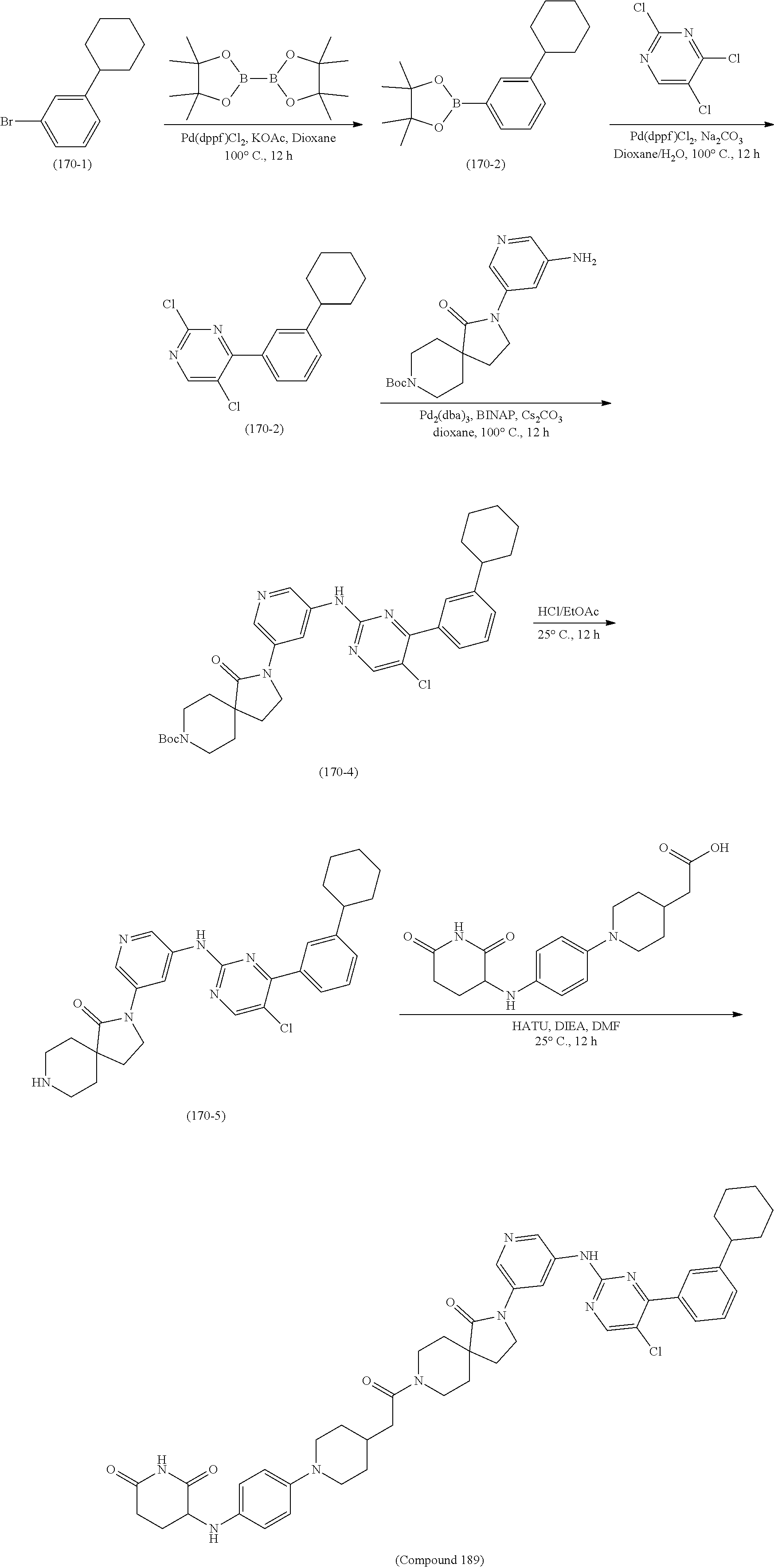

Synthesis of 2-(3-cyclohexylphenyl)-4,4,5,5-tetramethyl-1,3,2-dioxaborolane (170-2). A mixture of 1-bromo-3-cyclohexylbenzene (170-1) (1.00 g, 4.18 mmol, 1.00 eq), 4,4,4′,4′,5,5,5′,5′-octamethyl-2,2′-bi(1,3,2-dioxaborolane) (1.27 g, 5.02 mmol, 1.20 eq), Pd(dppf)Cl₂ (305 mg, 418 μmol, 0.100 eq) and KOAc (1.23 g, 12.5 mmol, 3.00 eq) in dioxane (10 mL) at 25° C. was degassed and purged with N₂ for 3 times, and then the mixture was stirred at 100° C. for 12 h under N₂ atmosphere. LCMS showed that the desired mass was detected. The reaction mixture was quenched by addition H₂O 10 ml at 25° C., and extracted with EtOAc (10 mL*3). The combined organic layers were washed with brine (10 mL*1), dried over Na₂SO₄, filtered and concentrated under reduced pressure. The residue was purified by column chromatography (SiO₂, Dichloromethane/Methanol=1/0 to 10/1) to give the title compound (170-2) (1.08 g, 2.61 mmol, 62.4% yield, 69.1% purity) as a green oil. LCMS (ES⁺): m/z 287.2 [M+H]⁺.

Synthesis of 2,5-dichloro-4-(3-cyclohexylphenyl)pyrimidine (170-3). A mixture of 2-(3-cyclohexylphenyl)-4,4,5,5-tetramethyl-1,3,2-dioxaborolane (170-2) (1.03 g, 3.60 mmol, 1.00 eq), 2,4,5-trichloropyrimidine (660 mg, 3.60 mmol, 1.00 eq), Pd(dppf)Cl₂ (263 mg, 359 μmol, 0.10 eq), Na₂CO₃ (1.14 g, 10.8 mmol, 3.00 eq) in dioxane (12 mL) and Water (4 mL) was degassed and purged with N₂ for 3 times, and then the mixture was stirred at 100° C. for 12 h under N₂ atmosphere. LCMS showed that the desired mass was detected. The reaction mixture was diluted with water 20 ml at 25° C., and extracted with EtOAc (20 mL*3). The combined organic layers were washed with brine 20 mL, dried over Na₂SO₄, filtered and the filtrate was concentrated under reduced pressure. The residue was purified by column chromatography (SiO₂, Petroleum ether/Ethyl acetate=1/0 to 5/1) to give the title compound (170-3) (747 mg, 1.59 mmol, 44.2% yield, 65.5% purity) as a green solid. LCMS (ES⁺): m/z 307.1 [M+H]⁺.

Synthesis of tert-butyl 2-(5-((5-chloro-4-(3-cyclohexylphenyl)pyrimidin-2-yl)amino)pyridin-3-yl)-1-oxo-2,8-diazaspiro[4.5]decane-8-carboxylate (170-4). A mixture of 2,5-dichloro-4-(3-cyclohexylphenyl)pyrimidine (170-3) (590 mg, 1.92 mmol, 1.00 eq), tert-butyl 2-(5-aminopyridin-3-yl)-1-oxo-2,8-diazaspiro[4.5]decane-8-carboxylate (665 mg, 1.92 mmol, 1.00 eq), Pd₂(dba)₃ (175 mg, 192 μmol, 0.100 eq), BINAP (119 mg, 192 μmol, 0.100 eq) and Cs₂CO₃ (1.88 g, 5.76 mmol, 3.00 eq) in dioxane (10 mL) at 25° C. was degassed and purged with N₂ for 3 times, and then the mixture was stirred at 100° C. for 12 h under N₂ atmosphere. LCMS showed the desired mass was detected. The reaction mixture was diluted with water 10 ml at 25° C., and extracted with EtOAc (10 mL*3). The combined organic layers were washed with brine (10 mL*1), dried over Na₂SO₄, filtered and the filtrate was concentrated under reduced pressure. The residue was purified by column chromatography (SiO₂, Petroleum ether/Ethyl acetate=1/0 to 1/1) to give the title compound (170-4) (564 mg, 747 μmol, 38.9% yield, 81.8% purity) as a yellow solid. LCMS (ES⁺): m/z 617.3 [M+H]⁺.

Synthesis of 2-(5-((5-chloro-4-(3-cyclohexylphenyl)pyrimidin-2-yl)amino)pyridin-3-yl)-2,8-diazaspiro[4.5]decan-1-one (170-5). A solution of 2-(5-((5-chloro-4-(3-cyclohexylphenyl)pyrimidin-2-yl)amino)pyridin-3-yl)-1-oxo-2,8-diazaspiro[4.5]decane-8-carboxylate (170-4) (564 mg, 913 μmol, 1.00 eq) in HCl/EtOAc (4 M, 8 mL) was stirred at 25° C. for 12 h. LCMS showed that the desired mass was detected. The reaction mixture was filtered and the filter cake was dried under high vacuum to give the title compound (170-5) (600 mg, crude) as a light yellow solid. LCMS (ES⁺): m/z 517.2 [M+H]⁺.

Synthesis of 3-((4-(4-(2-(2-(5-((5-chloro-4-(3-cyclohexylphenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 189). To a solution of 2-(5-((5-chloro-4-(3-cyclohexylphenyl)pyrimidin-2-yl)amino)pyridin-3-yl)-2,8-diazaspiro[4.5]decan-1-one (170-5) (100 mg, 193 μmol, 1.00 eq) in DMF (5 mL) were added 2-(1-(4-((2,6-dioxopiperidin-3-yl)amino)phenyl)piperidin-4-yl)acetic acid (66.8 mg, 193.4 μmol, 1.00 eq), HATU (110 mg, 290 μmol, 1.50 eq) and DIEA (74.9 mg, 580 μmol, 101 μL, 3.00 eq) at 25° C. The mixture was stirred at 25° C. for 12 h. LCMS showed that the desired mass was detected. The reaction mixture was filtered and the filtrate was concentrated under reduced pressure. The residue was purified by prep-HPLC (column: Phenomenex Luna C18 200*40 mm*10 um; mobile phase: [water (FA)-ACN]; gradient: 30%-60% B over 8 min) to give the title compound (Compound 189) (13.0 mg, 15.0 μmol, 7.77% yield, 97.5% purity) as a dark blue solid. LCMS (ES⁺): m/z 844.4 [M+H]⁺.

### Example 171. Synthesis of 3-((4-(1-(2-(2-(5-((5-chloro-4-(3-cyclohexylphenyl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 190)

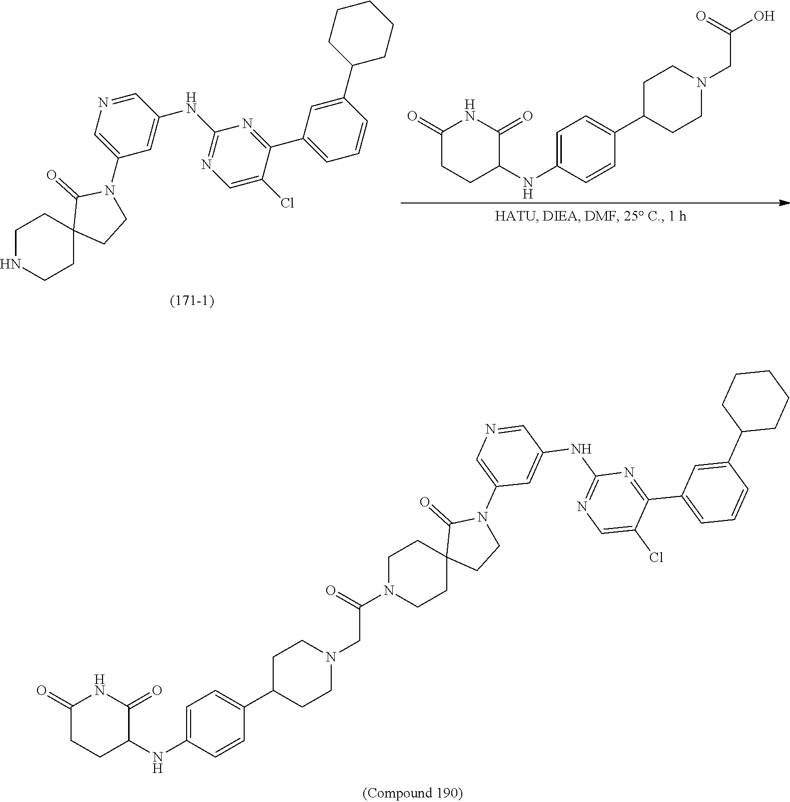

Synthesis of 3-((4-(1-(2-(2-(5-((5-chloro-4-(3-cyclohexylphenyl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 190). To a solution of 2-(5-((5-chloro-4-(3-cyclohexylphenyl)pyrimidin-2-yl)amino)pyridin-3-yl)-2,8-diazaspiro[4.5]decan-1-one (171-1) (100 mg, 193 μmol, 1.00 eq) in DMF (2 mL) were added 2-(4-(4-((2,6-dioxopiperidin-3-yl)amino)phenyl)piperidin-1-yl)acetic acid (66.8 mg, 193 μmol, 1.00 eq), HATU (110 mg, 290 μmol, 1.50 eq) and DIEA (74.9 mg, 580 μmol, 101 μL, 3.00 eq) at 25° C. The mixture was stirred at 25° C. for 1 h. LCMS showed that the desired mass was detected. The reaction mixture was filtered and the filtrate was concentrated under reduced pressure. The residue was purified by prep-HPLC (column: C18-6 100*30 mm*5 um; mobile phase: [water (FA)-ACN]; gradient: 25%-55% B over 8 min) to give the title compound (Compound 190) (45.2 mg, 52.0 μmol, 26.9% yield, 97.1% purity) as a light yellow solid. LCMS (ES⁺): m/z 844.4 [M+H]⁺.

### Example 172. Synthesis of 3-((4-(4-(2-(2-(5-((5-chloro-4-(3-cyclopentylphenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 191)

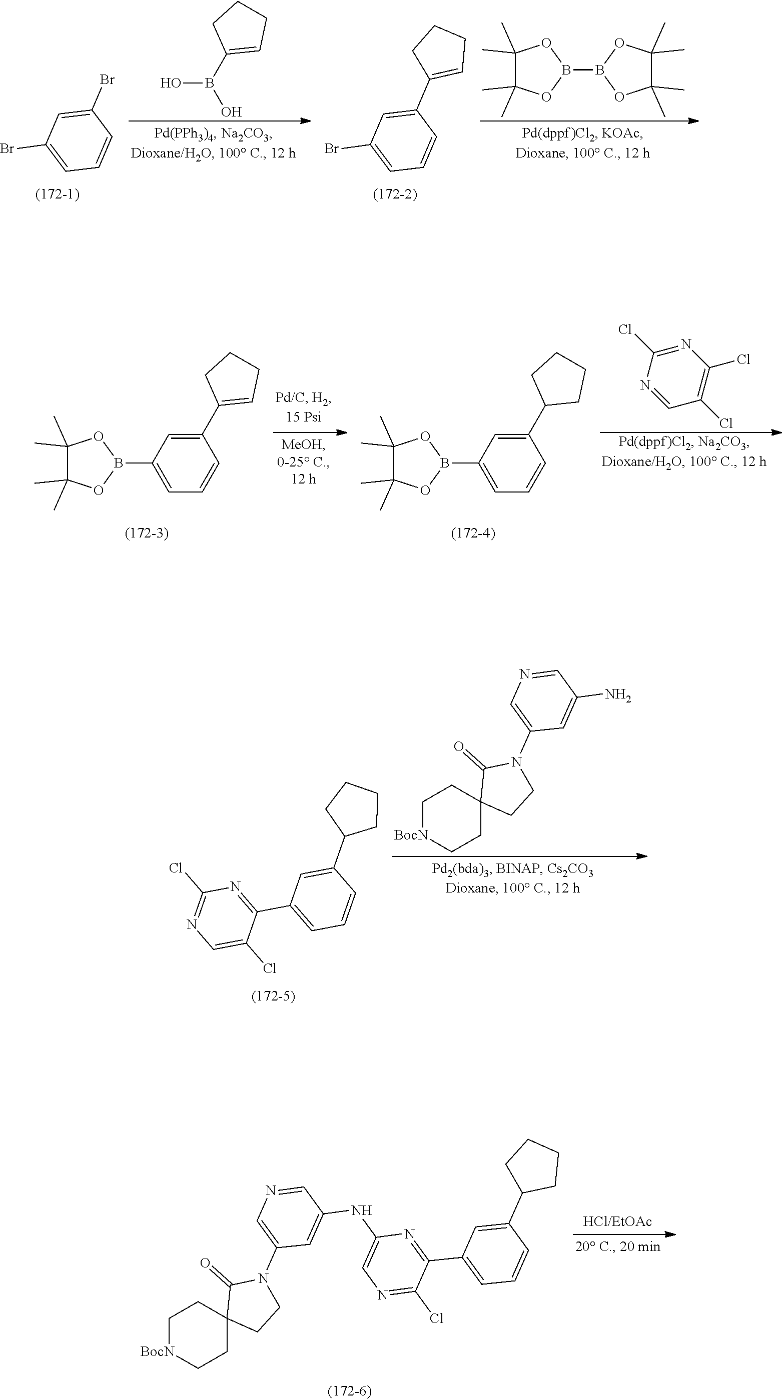

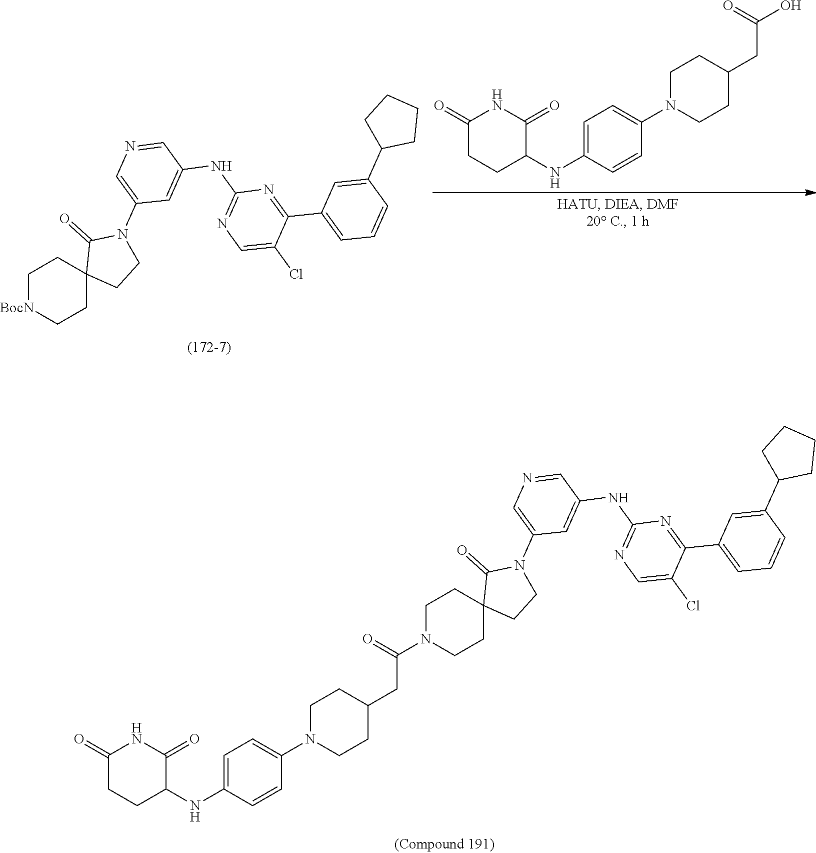

Synthesis of 1-bromo-3-(cyclopenten-1-yl) benzene (172-2). To a solution of 1,3-dibromobenzene (172-1) (5.00 g, 21.2 mmol, 2.56 mL, 1.00 eq), cyclopenten-1-ylboronic acid (2.37 g, 21.2 mmol, 1.00 eq), Na₂CO₃ (6.74 g, 63.6 mmol, 3.00 eq) and Pd(PPh₃)₄ (2.45 g, 2.12 mmol, 0.100 eq) in dioxane (50 mL) and H₂O (10 mL) was degassed and purged with N₂ at 20° C. for 3 times, and then the mixture was stirred at 100° C. for 12 h under N₂ atmosphere. TLC (Petroleum ether/Ethyl acetate=1/0, R_{f}=0.79) indicated 1,3-dibromobenzene (172-1) was consumed completely and one main new spot formed. H₂O 50 mL was added to the mixture, and extracted with EtOAc (50 mL*3). The combined organic layers were washed with brine (20 mL), dried over Na₂SO₄, filtered and the filtrate was concentrated under reduced pressure. The residue was purified by column chromatography (SiO₂, Petroleum ether/Ethyl acetate=0/1, R_{f}=0.79) to give the title compound (172-2) (9.20 g, crude) as a yellow oil. ¹H NMR (400 MHz, DMSO-d₆) δ=7.63-7.60 (m, 1H), 7.48-7.41 (m, 2H), 7.32-7.28 (m, 1H), 6.37 (br s, 1H), 2.76-2.57 (m, 3H), 1.99-1.86 (m, 3H).

Synthesis of 2-[3-(cyclopenten-1-yl)phenyl]-4,4,5,5-tetramethyl-1,3,2-dioxaborolane (172-3). To a solution of 1-bromo-3-(cyclopenten-1-yl)benzene (172-2) (4.00 g, 17.9 mmol, 1.00 eq), 4,4,5,5-tetramethyl-2-(4,4,5,5-tetramethyl-1,3,2-dioxaborolan-2-yl)-1,3,2-dioxaborolane (6.83 g, 26.9 mmol, 1.50 eq), KOAc (5.28 g, 53.7 mmol, 3.00 eq) and Pd(dppf)Cl₂ (1.31 g, 1.79 mmol, 0.100 eq) in dioxane (30 mL) was degassed and purged with N₂ at 20° C. for 3 times, and then the mixture was stirred at 100° C. for 12 h under N₂ atmosphere. LCMS the desired mass was detected. H₂O 20 mL was added to the mixture, and extracted with EtOAc (30 mL*3). The combined organic layers were washed with brine 10 mL, dried over Na₂SO₄, filtered and the filtrate was concentrated under reduced pressure. The residue was purified by column chromatography (SiO₂, Petroleum ether/Ethyl acetate=5/1, R_{f}=0.58) to give the title compound (172-3) (9.20 g, crude) as a white solid. LCMS (ES⁺): m/z 271.1 [M+H]⁺.

Synthesis of 2-(3-cyclopentylphenyl)-4,4,5,5-tetramethyl-1,3,2-dioxaborolane (172-4). To a solution of 2-[3-(cyclopenten-1-yl)phenyl]-4,4,5,5-tetramethyl-1,3,2-dioxaborolane (172-3) (3.20 g, 11.8 mmol, 1.00 eq) in MeOH (5 mL) was added 10% Pd/C (700 mg, 50.0% purity) at 0° C. under N₂ atmosphere. The suspension was degassed and purged with H₂ for 3 times. The mixture was stirred under H₂ (15 Psi) at 25° C. for 12 h. LCMS showed 2-[3-(cyclopenten-1-yl)phenyl]-4,4,5,5-tetramethyl-1,3,2-dioxaborolane (172-3) was consumed completely and the desired mass was detected. The reaction mixture was filtered and the filtrate was concentrated under reduce pressure to give the title compound (172-4) (2.20 g, 8.08 mmol, 68.2% yield) as a colourless oil. LCMS (ES⁺): m/z 273.0 [M+H]⁺.

Synthesis of 2,5-dichloro-4-(3-cyclopentylphenyl)pyrimidine (172-5). To a solution of 2-(3-cyclopentylphenyl)-4,4,5,5-tetramethyl-1,3,2-dioxaborolane (172-4)(1.00 g, 3.67 mmol, 1.00 eq), 2,4,5-trichloropyrimidine (741 mg, 4.04 mmol, 1.10 eq), Pd(dppf)Cl₂ (268 mg, 367 μmol, 0.100 eq) and Na₂CO₃ (1.17 g, 11.0 mmol, 3.00 eq) in dioxane (10 mL) and H₂O (2 mL) was degassed and purged with N₂ at 25° C. for 3 times, and then the mixture was stirred at 100° C. for 12 h under N₂ atmosphere. LCMS showed 2-(3-cyclopentylphenyl)-4,4,5,5-tetramethyl-1,3,2-dioxaborolane (172-4) was consumed completely and the desired mass was detected. H₂O 20 mL was added to the mixture, and extracted with EtOAc (20 mL*3). The combined organic layers were washed with brine 10 mL, dried over Na₂SO₄, filtered and the filtrate was concentrated under reduced pressure. The residue was purified by prep-TLC (SiO₂, Petroleum ether/Ethyl acetate=1/1, R_{f}=0.67) to give the title compound (172-5) (900 mg, 1.27 mmol, 34.6% yield, 41.4% purity) as a white solid. LCMS (ES⁺): m/z 293.0 [M+H]⁺.

Synthesis of tert-butyl 2-(5-((5-chloro-4-(3-cyclopentylphenyl)pyrimidin-2-yl)amino)pyridin-3-yl)-1-oxo-2,8-diazaspiro[4.5]decane-8-carboxylate (172-6). To a solution of 2,5-dichloro-4-(3-cyclopentylphenyl)pyrimidine (172-5) (700 mg, 2.39 mmol, 1.00 eq), tert-butyl 2-(5-amino-3-pyridyl)-1-oxo-2,8-diazaspiro[4.5]decane-8-carboxylate (827 mg, 2.39 mmol, 1.00 eq), Pd₂(dba)₃ (218 mg, 238 μmol, 0.100 eq), BINAP (148 mg, 238 μmol, 0.100 eq) and Cs₂CO₃ (1.94 g, 5.97 mmol, 2.50 eq) in 1,4-dioxane (8 mL) was degassed and purged with N₂ at 20° C. for 3 times, and then the mixture was stirred at 100° C. for 12 h under N₂ atmosphere. LCMS showed 2,5-dichloro-4-(3-cyclopentylphenyl)pyrimidine (172-5) was consumed completely and the desired mass was detected. H₂O 10 mL was added to the mixture, and extracted with EtOAc (10 mL*3). The combined organic layers were washed with brine (10 mL), dried over Na₂SO₄, filtered and the filtrate was concentrated under reduced pressure. The residue was purified by column chromatography (SiO₂, Petroleum ether/Ethyl acetate=0/1, R_{f}=0.27) to give the title compound (172-6) (500 mg, crude) as a brown solid. LCMS (ES⁺): m/z 603.2 [M+H]⁺.

Synthesis of 2-(5-((5-chloro-4-(3-cyclopentylphenyl)pyrimidin-2-yl)amino)pyridin-3-yl)-2,8-diazaspiro[4.5]decan-1-one (172-7). A solution of tert-butyl 2-[5-[[5-chloro-4-(3-pyrrolidin-1-ylphenyl)pyrimidin-2-yl]amino]-3-pyridyl]-1-oxo-2,8-diazaspiro[4.5]decane-8-carboxylate (172-6) (300 mg, 496 μmol, 1.00 eq) in HCl/EtOAc (4 M, 10.0 mL, 80.6 eq) was stirred at 20° C. for 20 min. LCMS showed tert-butyl 2-[5-[[5-chloro-4-(3-pyrrolidin-1-ylphenyl)pyrimidin-2-yl]amino]-3-pyridyl]-1-oxo-2,8-diazaspiro[4.5]decane-8-carboxylate (172-6) was consumed completely and the desired mass was detected. The reaction was concentrated under reduced pressure to give the title compound (172-7) (300 mg, crude) as a white solid. LCMS (ES⁺): m/z 503.2 [M+H]⁺.

Synthesis of 3-((4-(4-(2-(2-(5-((5-chloro-4-(3-cyclopentylphenyl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 191). To a solution of 2-[5-[[5-chloro-4-(3-cyclopentylphenyl)pyrimidin-2-yl]amino]-3-pyridyl]-2,8-diazaspiro[4.5]decan-1-one (172-7) (130 mg, 258 μmol, 1.00 eq) in DMF (3 mL) were added HATU (147 mg, 387 μmol, 1.50 eq), 2-[1-[4-[(2,6-dioxo-3-piperidyl)amino]phenyl]-4-piperidyl]acetic acid (107 mg, 310 μmol, 1.20 eq) and DIEA (167 mg, 1.29 mmol, 225 μL, 5.00 eq) at 20° C. The mixture was stirred at 20° C. for 1 h. LCMS showed 2-[5-[[5-chloro-4-(3-cyclopentylphenyl)pyrimidin-2-yl]amino]-3-pyridyl]-2,8-diazaspiro[4.5]decan-1-one (172-7) was consumed completely and the desired mass was detected. The reaction mixture was filtered and the filtrate concentrated under high vacuum. The residue was purified by prep-HPLC (Phenomenex Luna C18 75*30 mm*3 um; mobile phase: [H₂O (0.1% TFA)-ACN]; gradient: 20%-50% B over 8.0 min) to give the title compound (Compound 191) (9.3 mg, 11.2 μmol, 4.31% yield, 99.5% purity) as a green solid. LCMS (ES⁺): m/z 830.4 [M+H]⁺.

### Example 173. Synthesis of 3-((4-(1-(2-(2-(5-((5-chloro-4-(3-cyclopentylphenyl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 192)

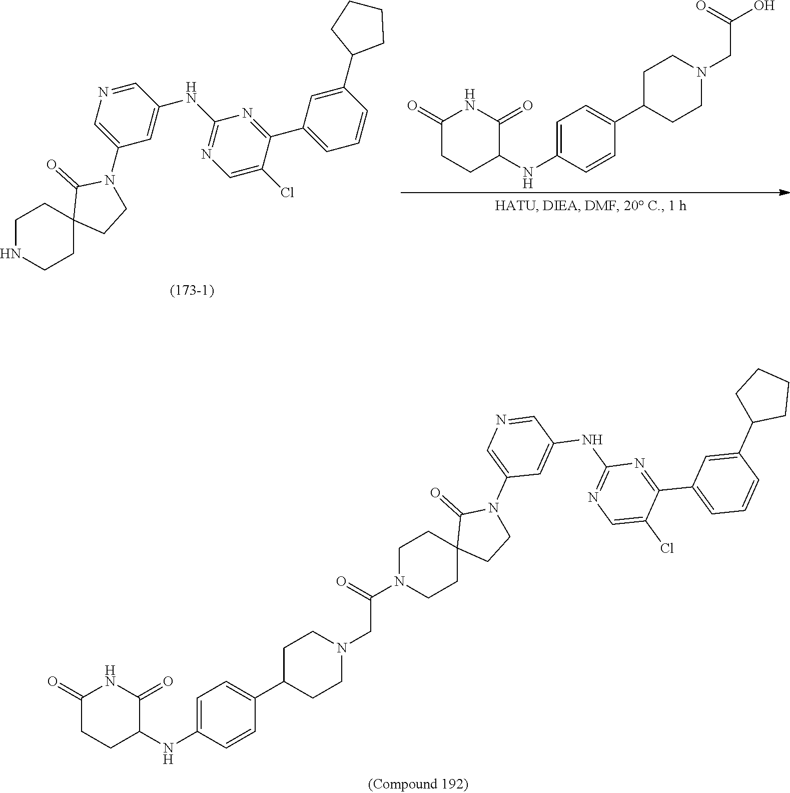

Synthesis of 3-((4-(1-(2-(2-(5-((5-chloro-4-(3-cyclopentylphenyl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 192). To a solution of 2-[5-[[5-chloro-4-(3-cyclopentylphenyl)pyrimidin-2-yl]amino]-3-pyridyl]-2,8-diazaspiro[4.5]decan-1-one (173-1) (130 mg, 258 μmol, 1.00 eq) in DMF (3 mL) were added HATU (147 mg, 387 μmol, 1.50 eq), 2-[4-[4-[(2,6-dioxo-3-piperidyl)amino]phenyl]-1-piperidyl]acetic acid (107 mg, 310 μmol, 1.20 eq) and DIEA (167 mg, 1.29 mmol, 225 μL, 5.00 eq) at 20° C. The mixture was stirred at 20° C. for 1 h. LCMS showed 2-[5-[[5-chloro-4-(3-cyclopentylphenyl)pyrimidin-2-yl]amino]-3-pyridyl]-2,8-diazaspiro[4.5]decan-1-one (173-1) was consumed completely and the desired mass was detected. The reaction mixture was filtered and the filtrate concentrated under high vacuum. The residue was purified by prep-HPLC (Phenomenex Luna C18 75*30 mm*3 um; mobile phase: [H2O (0.1% TFA)-ACN]; gradient: 20%-50% B over 8.0 min) to give the title compound (Compound 192) (51.1 mg, 60.5 μmol, 23.4% yield, 98.4% purity) as a gray solid. LCMS (ES⁺): m/z 830.4 [M+H]⁺.

### Example 174. Synthesis of 3-((4-(4-(2-(2-(5-((5-chloro-4-(1-(1-methylpiperidin-4-yl)-1H-pyrazol-4-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 193)

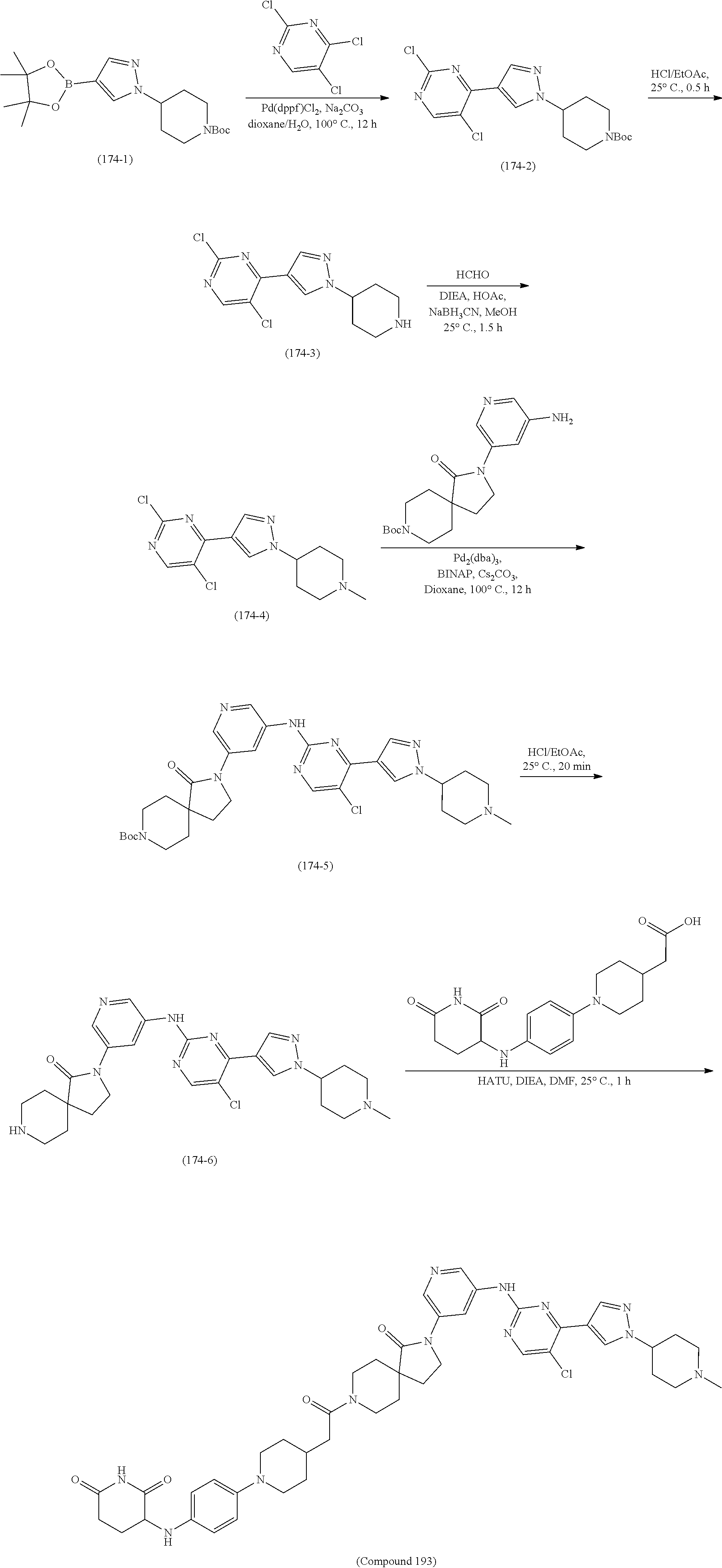

Synthesis of tert-butyl 4-(4-(2,5-dichloropyrimidin-4-yl)-1H-pyrazol-1-yl)piperidine-1-carboxylate (174-2). A mixture of tert-butyl 4-[4-(4,4,5,5-tetramethyl-1,3,2-dioxaborolan-2-yl)pyrazol-1-yl]piperidine-1-carboxylate (174-1) (5.00 g, 13.2 mmol, 1.00 eq), 2,4,5-trichloropyrimidine (2.92 g, 15.9 mmol, 1.20 eq), Pd(dppf)Cl₂ (969 mg, 1.33 mmol, 0.10 eq) and Na₂CO₃ (2.81 g, 26.5 mmol, 2.00 eq) in dioxane (45 mL) and H₂O (15 mL) was degassed and purged with N₂ for 3 times at 25° C., and then the mixture was stirred at 100° C. for 12 h under N₂ atmosphere. LCMS showed that the desired mass was detected. The reaction mixture was diluted with H₂O 100 mL and extracted with EtOAc (150 mL*3). The combined organic layers were washed with brine 10 mL, dried over Na₂SO₄, filtered and the filtrate was concentrated under reduced pressure. The residue was purified by column chromatography (SiO₂, Dichloromethane/Methanol=1/0 to 10/1) to give the title compound (174-2) (4.80 g, 11.8 mmol, 89.1% yield, 98.0% purity) as a brown oil. LCMS (ES⁺): m/z 342.0 [M−56+H]⁺.

Synthesis of 2,5-dichloro-4-[1-(4-piperidyl)pyrazol-4-yl]pyrimidine (174-3). A solution of tert-butyl 4-[4-(2,5-dichloropyrimidin-4-yl)pyrazol-1-yl]piperidine-1-carboxylate (174-2) (2.80 g, 7.03 mmol, 1.00 eq) in HCl/EtOAc (4 M, 30.0 mL) was stirred at 25° C. for 0.5 h. LCMS showed the desired mass was detected. The reaction mixture was concentrated under reduced pressure to give the title compound (174-3) (2.10 g, crude) as a yellow solid. LCMS (ES⁺): m/z 298.0 [M+H]⁺.

Synthesis of 2,5-dichloro-4-[1-(1-methyl-4-piperidyl)pyrazol-4-yl]pyrimidine (174-4). To a solution of formaldehyde (598 mg, 7.38 mmol, 549 μL, 1.10 eq) in MeOH (30 mL) was added DIEA (866 mg, 6.71 mmol, 1.17 mL, 1.00 eq) at 25° C. to adjust pH to 8. The mixture was stirred at 25° C. for 0.5 h. Then AcOH (402 mg, 6.71 mmol, 383 μL, 1.00 eq), 2,5-dichloro-4-[1-(4-piperidyl)pyrazol-4-yl]pyrimidine (174-3) (2.00 g, 6.71 mmol, 1.00 eq) and sodium; cyanoboranuide (1.26 g, 20.1 mmol, 3.00 eq) were added to the mixture at 25° C. The mixture was stirred at 25° C. for 1 h. LCMS showed the desired mass was detected. The reaction mixture was diluted with H₂O 20 mL and extracted with EtOAc (30 mL*3). The combined organic layers were dried over Na₂SO₄, filtered and concentrated under reduced pressure. The residue was purified by column chromatography (SiO₂, Dichloromethane/Methanol=1/0 to 10/1) to give the title compound (174-4) (1.10 g, 3.31 mmol, 49.3% yield, 94.0% purity) as a yellow oil. LCMS (ES⁺): m/z 312.1 [M+H]⁺.

Synthesis of tert-butyl 2-[5-[[5-chloro-4-[I-(1-methyl-4-piperidyl)pyrazol-4-yl]pyrimidin-2-yl]amino]-3-pyridyl]-1-oxo-2,8-diazaspiro[4.5]decane-8-carboxylate (174-5). A mixture of 2,5-dichloro-4-[1-(1-methyl-4-piperidyl)pyrazol-4-yl]pyrimidine (174-4) (600 mg, 1.92 mmol, 1.00 eq), tert-butyl 2-(5-amino-3-pyridyl)-1-oxo-2,8-diazaspiro[4.5]decane-8-carboxylate (798 mg, 2.31 mmol, 1.20 eq), Pd₂(dba)₃ (175 mg, 192 μmol, 0.100 eq), BINAP (119 mg, 192 μmol, 0.100 eq) and Cs₂CO₃ (1.88 g, 5.77 mmol, 3.00 eq) in dioxane (10 mL) was degassed and purged with N₂ for 3 times at 25° C., and then the mixture was stirred at 100° C. for 12 h under N₂ atmosphere. LCMS showed that the desired mass was detected. The reaction mixture was diluted with H₂O 20 mL and extracted with EtOAc (30 mL*3). The combined organic layers were dried over Na₂SO₄, filtered and concentrated under reduced pressure. The residue was purified by column chromatography (SiO₂, Dichloromethane/Methanol=1/0 to 5/1) to give the title compound (174-5) (530 mg, 681 μmol, 35.4% yield, 80.0% purity) as a brown oil. LCMS (ES⁺): m/z 622.4 [M+H]⁺.

Synthesis of 2-[5-[[5-chloro-4-[1-(1-methyl-4-piperidyl) pyrazol-4-yl]pyrimidin-2-yl]amino]-3-pyridyl]-2,8-diazaspiro[4.5]decan-1-one (174-6). A solution of tert-butyl 2-[5-[[5-chloro-4-[1-(1-methyl-4-piperidyl)pyrazol-4-yl]pyrimidin-2-yl]amino]-3-pyridyl]-1-oxo-2,8-diazaspiro[4.5]decane-8-carboxylate (174-5) (500 mg, 803 μmol, 1.00 eq) in HCl/EtOAc (4 M, 3 mL) was stirred at 25° C. for 20 min. LCMS showed that the desired mass was detected. The reaction mixture was concentrated under reduced pressure to give the title compound (174-6) (580 mg, crude) as a yellow solid. LCMS (ES⁺): m/z 522.3 [M+H]⁺.

Synthesis of 3-((4-(4-(2-(2-(5-((5-chloro-4-(1-(1-methylpiperidin-4-yl)-1H-pyrazol-4-yl)pyrimidin-2-yl)amino)pyridin-3-yl)-1-oxo-2,8-diazaspiro[4.5]decan-8-yl)-2-oxoethyl)piperidin-1-yl)phenyl)amino)piperidine-2,6-dione (Compound 193). To a solution of 2-[5-[[5-chloro-4-[1-(1-methyl-4-piperidyl)pyrazol-4-yl]pyrimidin-2-yl]amino]-3-pyridyl]-2,8-diazaspiro[4.5]decan-1-one (174-6) (150 mg, 287 μmol, 1.00 eq) in DMF (2 mL) was added DIEA (40.8 mg, 316 μmol, 55.0 μL, 1.10 eq) to adjust pH to 7-8. Then 2-[1-[4-[(2,6-dioxo-3-piperidyl)amino]phenyl]-4-piperidyl]acetic acid (109 mg, 316 μmol, 1.10 eq), DIEA (37.1 mg, 287 μmol, 50.0 μL, 1.00 eq) and HATU (131 mg, 344 μmol, 1.20 eq) was added to the above mixture at 25° C. The mixture was stirred at 25° C. for 1 h. LCMS showed that the desired mass was detected. The residue was purified by prep-HPLC (column: Phenomenex Luna C18 75*30 mm*3 um; mobile phase: [water (FA)-ACN]; gradient: 1%-35% B over 8 min) to give the title compound (Compound 193) (25.8 mg, 29.9 μmol, 10.4% yield, 99.3% purity, 0.14 FA) as a blue solid. LCMS (ES⁺): m/z 849.4 [M+H]⁺.

### Example 175. Synthesis of 3-((4-(1-(2-(2-(5-((5-chloro-4-(1-(1-methylpiperidin-4-yl)-1H-pyrazol-4-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 194)

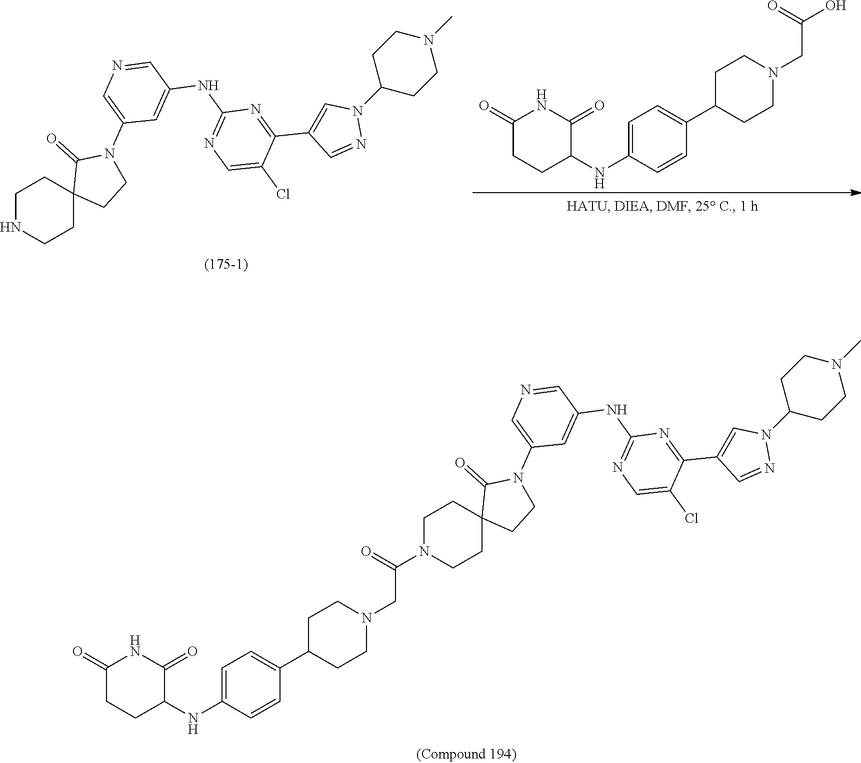 [SMILES: CN1CCC(n2cc(-c3nc(Nc4cncc(N5CCC6(CCNCC6)C5=O)c4)ncc3Cl)cn2)CC1 ; O=C(O)CN1CCC(c2ccc(NC3CCC(=O)NC3=O)cc2)CC1 ; CN1CCC(n2cc(-c3nc(Nc4cncc(N5CCC6(CCN(C(=O)CN7CCC(c8ccc(NC9CCC(=O)NC9=O)cc8)CC7)CC6)C5=O)c4)ncc3Cl)cn2)CC1]

Synthesis of 3-((4-(1-(2-(2-(5-((5-chloro-4-(1-(1-methylpiperidin-4-yl)-1H-pyrazol-4-yl)pyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 194). To a solution of 2-[5-[[5-chloro-4-[1-(1-methyl-4-piperidyl)pyrazol-4-yl]pyrimidin-2-yl]amino]-3-pyridyl]-2,8-diazaspiro[4.5]decan-1-one (175-1) (150 mg, 287 μmol, 1.00 eq) in DMF (2 mL) was added DIEA (40.8 mg, 316 μmol, 55.0 μL, 1.10 eq) to adjust pH to 7-8. Then 2-(4-(4-((2,6-dioxopiperidin-3-yl)amino)phenyl)piperidin-1-yl)acetic acid (109 mg, 316 μmol, 1.10 eq) and HATU (131 mg, 344 μmol, 1.20 eq) were added to the above mixture. The mixture was stirred at 25° C. for 1 h. LCMS showed that 2-[5-[[5-chloro-4-[1-(1-methyl-4-piperidyl)pyrazol-4-yl]pyrimidin-2-yl]amino]-3-pyridyl]-2,8-diazaspiro[4.5]decan-1-one (175-1) was consumed completely and the desired mass was detected. The residue was purified by prep-HPLC (column: Phenomenex C18 75*30 mm*3 um; mobile phase: [water (NH₃H₂O+NH₄HCO₃)-ACN]; gradient: 35%-55% B over 8 min) to give the title compound (Compound 194) (32.5 mg, 36.6 μmol, 12.7% yield, 95.8% purity) as a white solid. LCMS (ES⁺): m/z 849.4 [M+H]⁺.

### Example 176. Synthesis of 3-(5-((5-(2-(5-((5-chloro-4-(3-(pyridin-2-yl)phenyl)pyrimidin-2-yl)amino)pyridin-3-yl)-1-oxo-2,8-diazaspiro[4.5]decan-8-yl)-5-oxopentyl)amino)-1-oxoisoindolin-2-yl)piperidine-2,6-dione (Compound 195)

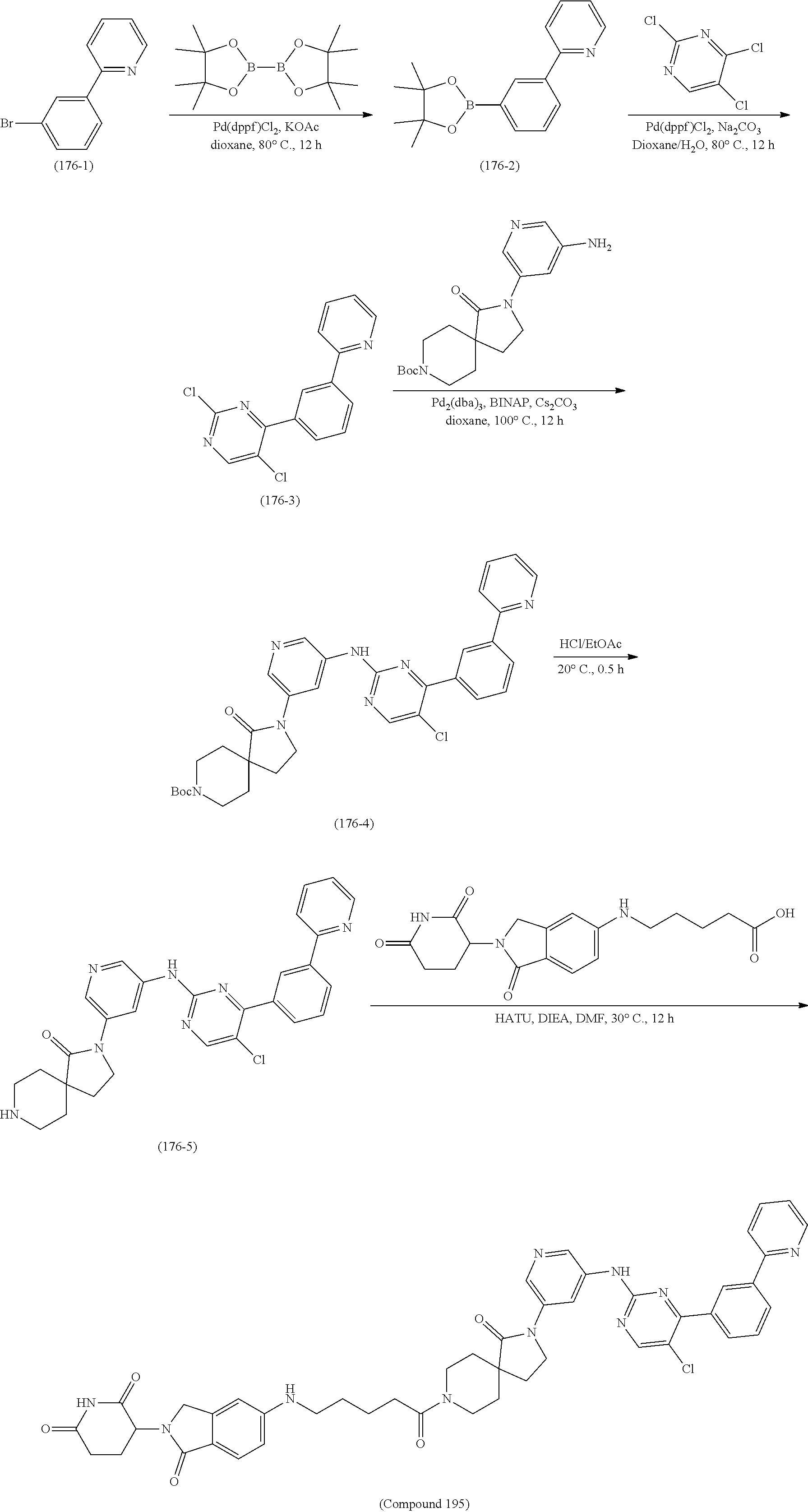

Synthesis of 2-(3-(4,4,5,5-tetramethyl-1,3,2-dioxaborolan-2-yl)phenyl)pyridine (176-2). To a solution of 2-(3-bromophenyl)pyridine (176-1) (2.00 g, 8.54 mmol, 1.00 eq) and 4,4,5,5-tetramethyl-2-(4,4,5,5-tetramethyl-1,3,2-dioxaborolan-2-yl)-1,3,2-dioxaborolane (3.25 g, 12.8 mmol, 1.50 eq) in dioxane (50.0 mL) were added Pd(dppf)Cl₂ (1.25 g, 1.71 mmol, 0.200 eq) and KOAc (2.52 g, 25.6 mmol, 3.00 eq) at 20° C. The mixture was stirred at 80° C. for 12 h. LCMS showed that 2-(3-bromophenyl)pyridine (176-1) was consumed completely and the desired mass was detected. H₂O 30 mL was added to the mixture, and then extracted with EtOAc (30 mL*3). The combined organic layers were washed with brine 30 mL, dried over Na₂SO₄, filtered and concentrated under reduced pressure. The residue was purified by column chromatography (SiO₂, Petroleum ether/Ethyl acetate=5/1 to 5/1) to give the title compound (176-2) (2.40 g, 7.34 mmol, 85.9% yield, 86.0% purity) as a white solid. LCMS (ES⁺): m/z 281.9 [M+H]⁺.

Synthesis of 2,5-dichloro-4-(3-(pyridin-2-yl)phenyl)pyrimidine (176-3). To a solution of 2-[3-(4,4,5,5-tetramethyl-1,3,2-dioxaborolan-2-yl)phenyl]pyridine (176-2) (1.40 g, 4.98 mmol, 1.00 eq) and 2,4,5-trichloropyrimidine (1.00 g, 5.48 mmol, 1.10 eq) in dioxane (50.00 mL) and H₂O (10.00 mL) were added Pd(dppf)Cl₂ (728 mg, 995 μmol, 0.200 eq) and Na₂CO₃ (1.58 g, 14.9 mmol, 3.00 eq) at 20° C. The mixture was stirred at 80° C. for 12 h. LCMS showed 2-[3-(4,4,5,5-tetramethyl-1,3,2-dioxaborolan-2-yl)phenyl]pyridine (176-2) was consumed completely and the desired mass was detected. H₂O 10 mL was added to the mixture, and then extracted with EtOAc (10 mL*3). The combined organic layers were washed with brine 10 mL, dried over Na₂SO₄, filtered and concentrated under reduced pressure. The residue was purified by column chromatography (SiO₂, Petroleum ether/Ethyl acetate=3/1 to 3/1) to give the title compound (176-3) (800 mg, 2.55 mmol, 51.2% yield, 96.3% purity) as a white solid. LCMS (ES⁺): m/z 301.9 [M+H]⁺.

Synthesis of tert-butyl 2-(5-((5-chloro-4-(3-(pyridin-2-yl)phenyl)pyrimidin-2-yl)amino)pyridin-3-yl)-1-oxo-2,8-diazaspiro[4.5]decane-8-carboxylate (176-4). To a solution of 2,5-dichloro-4-[3-(2-pyridyl)phenyl]pyrimidine (176-3) (400 mg, 1.32 mmol, 1.00 eq) and tert-butyl 2-(5-amino-3-pyridyl)-1-oxo-2,8-diazaspiro[4.5]decane-8-carboxylate (458 mg, 1.32 mmol, 1.00 eq) in dioxane (5 mL) were added BINAP (82.4 mg, 132 μmol, 0.100 eq), Cs₂CO₃ (1.29 g, 3.97 mmol, 3.00 eq) and Pd₂(dba)₃ (121 mg, 132 μmol, 0.100 eq) at 20° C. The mixture was stirred at 100° C. for 12 h under N₂ atmosphere. LCMS showed 2,5-dichloro-4-[3-(2-pyridyl)phenyl]pyrimidine (176-3) was consumed completely and the desired mass was detected. H₂O 10 mL was added to the mixture, and extracted with EtOAc (10 mL*3). The combined organic layers were washed with brine 10 mL, dried over Na₂SO₄, filtered and concentrated under reduced pressure. The residue was purified by prep-TLC (SiO₂, Petroleum ether/Ethyl acetate=5/1) to give the title compound (176-4) (490 mg, 558 μmol, 42.2% yield, 69.8% purity) as a white solid. LCMS (ES⁺): m/z 612.4 [M+H]⁺.

Synthesis of 2-(5-((5-chloro-4-(3-(pyridin-2-yl)phenyl)pyrimidin-2-yl)amino)pyridin-3-yl)-2,8-diazaspiro[4.5]decan-1-one (176-5). A solution of tert-butyl 2-[5-[[5-chloro-4-[3-(2-pyridyl)phenyl]pyrimidin-2-yl]amino]-3-pyridyl]-1-oxo-2,8-diazaspiro[4.5]decane-8-carboxylate (176-4) (200 mg, 326 μmol, 1.00 eq) in HCl/EtOAc (10 mL) was stirred at 20° C. for 0.5 h. LCMS showed tert-butyl 2-[5-[[5-chloro-4-[3-(2-pyridyl)phenyl]pyrimidin-2-yl]amino]-3-pyridyl]-1-oxo-2,8-diazaspiro[4.5]decane-8-carboxylate (176-4) was consumed completely and the desired mass was detected. The reaction mixture was filtered and the filtrate concentrated under high vacuum to give the title compound (176-5) (200 mg, crude) as a white solid. LCMS (ES⁺): m/z 512.3 [M+H]⁺.

Synthesis of 3-(5-((5-(2-(5-((5-chloro-4-(3-(pyridin-2-yl)phenyl)pyrimidin-2-yl)amino)pyridin-3-yl)-1-oxo-2,8-diazaspiro[4.5]decan-8-yl)-5-oxopentyl)amino)-1-oxoisoindolin-2-yl)piperidine-2,6-dione (Compound 195). To a solution of 2-[5-[[5-chloro-4-[3-(2-pyridyl)phenyl]pyrimidin-2-yl]amino]-3-pyridyl]-2,8-diazaspiro[4.5]decan-1-one (176-5) (200 mg, 390 μmol, 1.00 eq) and 5-[[2-(2,6-dioxo-3-piperidyl)-1-oxo-isoindolin-5-yl]amino]pentanoic acid (140 mg, 390 μmol, 1.00 eq) in DMF (5 mL) were added HATU (222.79 mg, 585 μmol, 1.50 eq) and DIEA (151 mg, 1.17 mmol, 204 μL, 3.00 eq) at 20° C. The mixture was stirred at 30° C. for 12 h. TLC (Petroleum ether/Ethyl acetate=5/1) indicated that one new spot formed. H₂O 10 mL was added to the mixture, and then extracted with EtOAc (10 mL*3). The combined organic layers were washed with brine 10 mL, dried over Na₂SO₄, filtered and concentrated under reduced pressure. The residue was purified by prep-HPLC to give the title compound (Compound 195) (35.1 mg, 38.0 μmol, 9.74% yield, 92.4% purity) as a brown solid. LCMS (ES⁺): m/z 853.3 [M+H]⁺.

### Example 177. Synthesis of 1-(5-((5-chloro-4-(3,4-dihydroquinolin-1(2H)-yl)pyrimidin-2-yl)amino)pyridin-3-yl)pyrrolidin-2-one (Compound 127)

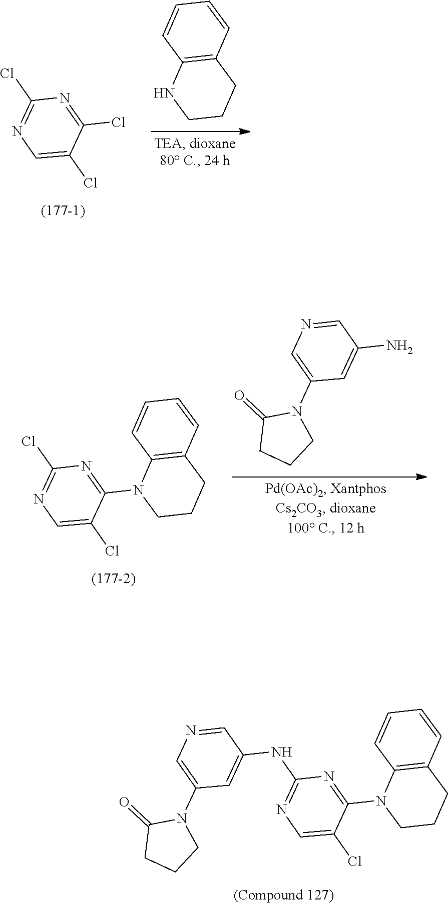 [SMILES: Clc1ncc(Cl)c(Cl)n1 ; c1ccc2c(c1)CCCN2 ; Clc1ncc(Cl)c(N2CCCc3ccccc32)n1 ; Nc1cncc(N2CCCC2=O)c1 ; O=C1CCCN1c1cncc(Nc2ncc(Cl)c(N3CCCc4ccccc43)n2)c1]

Synthesis of 1-(2,5-dichloropyrimidin-4-yl)-1,2,3,4-tetrahydroquinoline (177-2). To a solution of 2,4,5-trichloropyrimidine (177-1) (300 mg, 1.64 mmol, 1.00 eq) in dioxane (4 mL) were added 1,2,3,4-tetrahydroquinoline (218 mg, 1.64 mmol, 1.00 eq) and TEA (332 mg, 3.28 mmol, 457 μL, 2.00 eq) at 25° C., the mixture was stirred at 25° C. for 12 h. LCMS showed 1,2,3,4-tetrahydroquinoline was remaining, but no desired mass was detected. Then the mixture was stirred at 80° C. for 12 h. LCMS showed that the desired mass was detected. The reaction mixture was concentrated under reduced pressure to remove solvent. The residue was purified by prep-TLC (SiO₂, Petroleum ether/Ethyl acetate=10/1) to give the title compound (177-2) (320 mg, 571 umol, 34.8% yield, 50.0% purity) as a white solid. LCMS (ES⁺): m/z 280.1 [M+H]⁺

Synthesis of 1-(5-((5-chloro-4-(3,4-dihydroquinolin-1(2H)-yl)pyrimidin-2-yl)amino)pyridin-3-yl)pyrrolidin-2-one (Compound 127). To a solution of 1-(5-amino-3-pyridyl)pyrrolidine-2-one (145 mg, 571 umol, 70.0% purity, 1.00 eq) in dioxane (2 mL) were added 1-(2,5-dichloropyrimidin-4-yl)-1,2,3,4-tetrahydroquinoline (177-2) (320 mg, 571 umol, 50.0% purity, 1.00 eq), Xantphos (9.91 mg, 17.1 umol, 0.0300 eq), Cs₂CO₃ (372 mg, 1.14 mmol, 2.00 eq) and Pd(OAc)₂ (6.41 mg, 28.6 umol, 0.0500 eq) at 20° C. under N₂ atmosphere, the mixture was stirred at 100° C. for 12 h. LCMS showed 1-(2,5-dichloropyrimidin-4-yl)-1,2,3,4-tetrahydroquinoline (177-2) was consumed completely and the desired mass was detected. The reaction mixture was concentrated under reduced pressure to remove solvent. The residue was purified by prep-HPLC (column: Phenomenex Luna C18 75*30 mm*3 um; mobile phase: [water (FA)-ACN]; B %: 20%-60%, 8 min) to give the title compound (Compound 127) (31.7 mg, 68.9 umol, 12.1% yield, 91.5% purity) as a white solid. LCMS (ES⁺): m/z 421.1 [M+H]⁺.

### Example 178. Synthesis of 1-(5-((5-chloro-4-(3,4-dihydroisoquinolin-2(1H)-yl)pyrimidin-2-yl)amino)pyridin-3-yl)pyrrolidin-2-one (Compound 128)

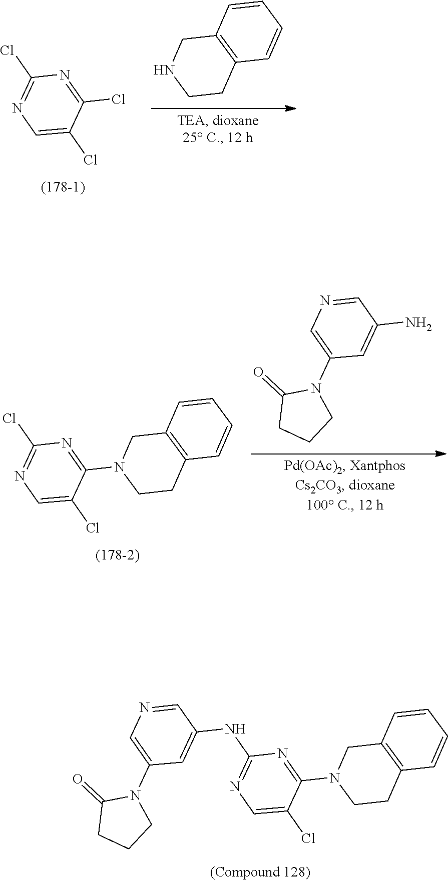 [SMILES: Clc1ncc(Cl)c(Cl)n1 ; c1ccc2c(c1)CCNC2 ; Clc1ncc(Cl)c(N2CCc3ccccc3C2)n1 ; Nc1cncc(N2CCCC2=O)c1 ; O=C1CCCN1c1cncc(Nc2ncc(Cl)c(N3CCc4ccccc4C3)n2)c1]

Synthesis of 2-(2,5-dichloropyrimidin-4-yl)-1,2,3,4-tetrahydroisoquinoline (178-2). To a solution of 2,4,5-trichloropyrimidine (178-1) (300 mg, 1.64 mmol, 1.00 eq) in dioxane (4 mL) were added 1,2,3,4-tetrahydroisoquinoline (218 mg, 1.64 mmol, 206 μL, 1.00 eq) and TEA (331 mg, 3.27 mmol, 455 μL, 2.00 eq) at 25° C., the mixture was stirred at 25° C. for 12 h. LCMS showed the desired mass was detected. The reaction mixture was concentrated under reduced pressure to remove solvent. The residue was purified by prep-TLC (SiO₂, Petroleum ether:Ethyl acetate=10:1) to give the title compound (178-2) (380 mg, 1.36 mmol, 82.9% yield) as a white solid. LCMS (ES⁺): m/z 280.1 [M+H]⁺.

Synthesis of 1-(5-((5-chloro-4-(3,4-dihydroisoquinolin-2(1H)-yl)pyrimidin-2-yl)amino)pyridin-3-yl)pyrrolidine-2-one (Compound 128). To a solution of 2-(2,5-dichloropyrimidin-4-yl)-1,2,3,4-tetrahydroisoquinoline (178-2) (380 mg, 1.36 mmol, 1.00 eq) in dioxane (5 mL) were added 1-(5-amino-3-pyridyl)pyrrolidine-2-one (240 mg, 1.36 mmol, 1.00 eq), Xantphos (23.6 mg, 40.7 umol, 0.0300 eq), Cs₂CO₃ (884 mg, 2.71 mmol, 2.00 eq) and Pd(OAc)₂ (15.2 mg, 67.8 umol, 0.0500 eq) at 25° C. under N₂ atmosphere, the mixture was stirred at 100° C. for 12 h. LCMS showed (2,5-dichloropyrimidin-4-yl)-1,2,3,4-tetrahydroisoquinoline (178-2) was consumed completely and the desired mass was detected. The reaction mixture was concentrated under reduced pressure to remove solvent. The residue was purified by prep-HPLC (column: Phenomenex Luna C18 75*30 mm*3 um; mobile phase: [water (TFA)-ACN]; B %: 25%-55%, 8 min) to give the title compound (Compound 128) (198 mg, 461 umol, 34.0% yield, 97.9% purity) as a white solid. LCMS (ES⁺): m/z 421.1 [M+H]⁺.

### Example 179. Synthesis of 1-(5-((5-chloro-4-(indolin-1-yl)pyrimidin-2-yl)amino)pyridin-3-yl)pyrrolidin-2-one (Compound 129)

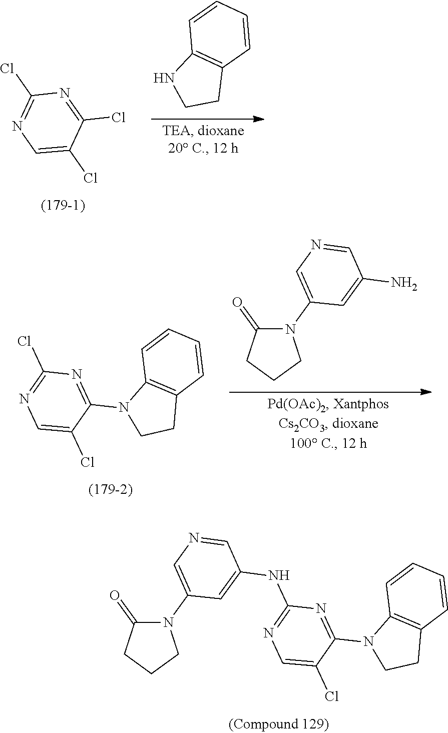

Synthesis of 1-(2,5-dichloropyrimidin-4-yl)indoline (179-2). To a solution of 2,4,5-trichloropyrimidine (179-1) (300 mg, 1.64 mmol, 1.00 eq) and indoline (195 mg, 1.64 mmol, 184 μL, 1.00 eq) in dioxane (3 mL) was added TEA (331 mg, 3.27 mmol, 455 μL, 2.00 eq) at 20° C., then the mixture was stirred at 20° C. for 12 h. LCMS showed the desired target MS was detected. The mixture was concentrated in vacuo, H₂O (6 mL) was added to the mixture and the mixture was extracted with EtOAc (6 mL*3), the combined organic layers were washed with saturated aqueous NaCl solution (6 mL*3), dried over Na₂SO₄, filtered and the filtrate was concentrated in vacuo to give the crude product. The crude product was purified by prep-TLC (SiO₂, PE/EtOAc=3/1) to give the title compound (179-2) (260 mg, 977 umol, 59.7% yield) as a yellow solid. LCMS (ES⁺): m/z 266.0 [M+H]⁺.

Synthesis of 1-(5-((5-chloro-4-(indolin-1-yl)pyrimidin-2-yl)amino)pyridin-3-yl)pyrrolidin-2-one (Compound 129). To a solution of 1-(2,5-dichloropyrimidin-4-yl)indoline (179-2) (130 mg, 488 umol, 1.00 eq) and 1-(5-aminopyridin-3-yl)pyrrolidin-2-one (124 mg, 488 umol, 70% purity, 1.00 eq) in dioxane (2 mL) were added Cs₂CO₃ (318 mg, 977 umol, 2.00 eq), Xantphos (8.48 mg, 14.7 umol, 0.0300 eq) and Pd(OAc)₂ (5.48 mg, 24.4 umol, 0.0500 eq) at 20° C. The mixture was stirred at 100° C. for 12 h under N₂ atmosphere. LCMS showed 1-(2,5-dichloropyrimidin-4-yl)indoline (179-2) was consumed, desired target MS was detected. H₂O (2 mL) was added to the mixture and the mixture was extracted with EtOAc (2 mL*3), the combined organic layers were washed with saturated aqueous NaCl solution (2 mL*3), dried over Na₂SO₄, filtered and the filtrate was concentrated in vacuo. The residue was purified by prep-HPLC (column: Phenomenex Luna C18 75*30 mm*3 um; mobile phase: [water (FA)-ACN]; B %: 25%-50%, 8 min) to give the title compound (Compound 129) (32.6 mg, 78.5 umol, 16.1% yield, 98.0% purity) as a white solid. LCMS (ES⁺): m/z 407.0 [M+H]⁺.

### Example 180. Synthesis of 1-(5-((5-chloro-4-(isoindolin-2-yl)pyrimidin-2-yl)amino)pyridin-3-yl)pyrrolidin-2-one (Compound 130)

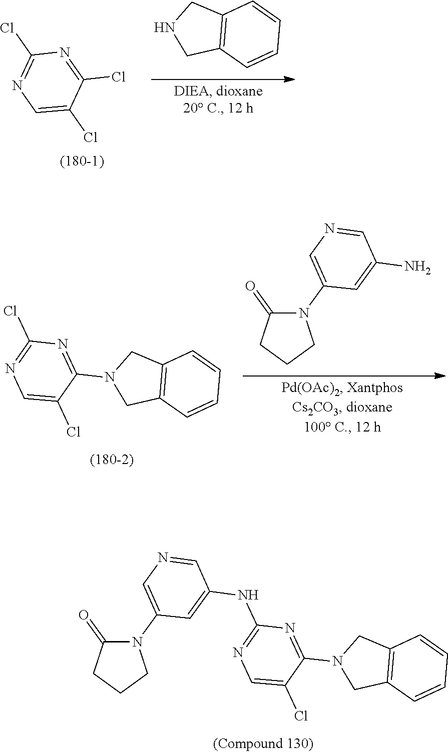 [SMILES: Clc1ncc(Cl)c(Cl)n1 ; c1ccc2c(c1)CNC2 ; Clc1ncc(Cl)c(N2Cc3ccccc3C2)n1 ; Nc1cncc(N2CCCC2=O)c1 ; O=C1CCCN1c1cncc(Nc2ncc(Cl)c(N3Cc4ccccc4C3)n2)c1]

Synthesis of 2-(2,5-dichloropyrimidin-4-yl)isoindoline (180-2). To a solution of 2,4,5-trichloropyrimidine (180-1) (300 mg, 1.64 mmol, 1.00 eq) and isoindoline (156 mg, 1.31 mmol, 148 μL, 0.800 eq) in dioxane (3 mL) was added TEA (331 mg, 3.27 mmol, 455 μL, 2.00 eq) at 20° C., then the mixture was stirred at 20° C. for 12 h. LCMS showed that the desired mass was detected. To the mixture was added H₂O (5 mL) and the mixture was extracted with EtOAc (5 mL*3). The combined organic layers were washed with saturated aqueous NaCl solution (5 mL*3), dried over Na₂SO₄, filtered and the filtrate was concentrated in vacuo. The mixture was purified by prep-TLC (SiO₂, Petroleum ether/Ethyl acetate=1/5) to give the title compound (180-2) (200 mg, 752 umol, 46.0% yield) as a white solid. LCMS (ES⁺): m/z 266.0 [M+H]⁺.

Synthesis of 1-(5-((5-chloro-4-(isoindolin-2-yl)pyrimidin-2-yl)amino)pyridin-3-yl)pyrrolidin-2-one (Compound 130). To a solution of 2-(2,5-dichloropyrimidin-4-yl)isoindoline (180-2) (160 mg, 601 umol, 1.00 eq) and 1-(5-amino-3-pyridyl)pyrrolidin-2-one (152 mg, 601 umol, 70% purity, 1.00 eq) in dioxane (3 mL) were added Cs₂CO₃ (392 mg, 1.20 mmol, 2.00 eq), Xantphos (17.4 mg, 30.1 umol, 0.0500 eq) and Pd(OAc)₂ (6.75 mg, 30.1 umol, 0.0500 eq) at 20° C. The mixture was stirred at 100° C. for 12 h under N₂ atmosphere. LCMS showed that 2-(2,5-dichloropyrimidin-4-yl)isoindoline (180-2) was consumed and the desired mass was detected. To the mixture was added H₂O (5 mL) and the mixture was extracted with EtOAc (5 mL*3). The combined organic layers were washed with saturated aqueous NaCl solution (10 mL*3), dried over Na₂SO₄, filtered and the filtrate was concentrated in vacuo. This mixture was purified by acidic prep-HPLC (column: Phenomenex Luna C18 200*40 mm*10 um; mobile phase: [water (FA)-ACN]; B %: 20%-60%, 8 min) to give the title compound (Compound 130) (3.80 mg, 9.21 umol, 1.53% yield, 98.6% purity) as white solid. LCMS (ES⁺): m/z 407.0 [M+H]⁺.

### Example 181. Synthesis of 1-(5-((5-chloro-4-(3-phenoxyphenyl)pyrimidin-2-yl)amino)pyridin-3-yl)pyrrolidin-2-one (Compound 142)

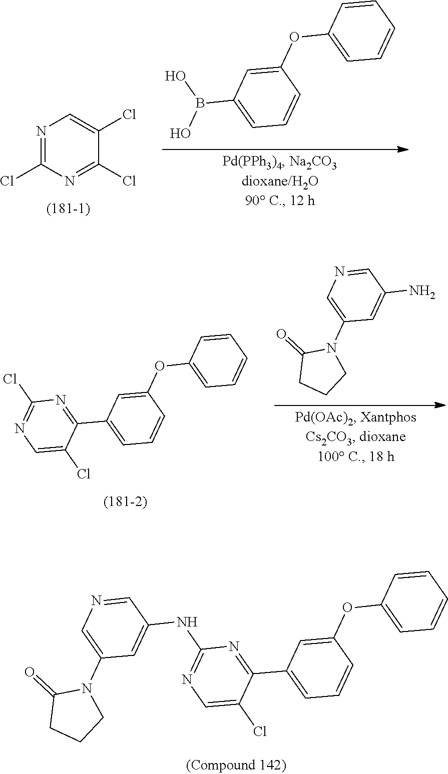

Synthesis of 2,5-dichloro-4-(3-phenoxyphenyl)pyrimidine (181-2). To a solution of (3-phenoxyphenyl)boronic acid (300 mg, 1.40 mmol, 326 μL, 1.00 eq) in dioxane (8 mL) and H₂O (0.8 mL) were added 2,4,5-trichloropyrimidine (181-1) (514 mg, 2.80 mmol, 2.00 eq), Na₂CO₃ (297 mg, 2.80 mmol, 2.00 eq) and Pd(PPh₃)₄ (32.4 mg, 28.0 umol, 0.0200 eq) at 25° C. under N₂ atmosphere. The mixture was stirred at 90° C. for 12 h. LCMS showed the desired mass was detected. Then H₂O (8 mL) was added to the mixture at 25° C. The reaction mixture was extracted with EtOAc (5 mL*3). The organic layers were dried over anhydrous sodium sulfate, filtered and the filtrate was concentrated under reduced to give a residue. The residue was purified by prep-TLC (SiO₂, Petroleum ether:Ethyl acetate=5:1) to give the title compound (181-2) (376 mg, 1.19 mmol, 84.6% yield) as a yellow oil. LCMS (ES⁺): m/z 317.1 [M+H]⁺.

Synthesis of 1-(5-((5-chloro-4-(3-phenoxyphenyl)pyrimidin-2-yl)amino)pyridin-3-yl)pyrrolidine-2-one (Compound 142). To a solution of 2,5-dichloro-4-(3-phenoxyphenyl)pyrimidine (181-2) (150 mg, 473 umol, 1.00 eq) in dioxane (5 mL) were added 1-(5-amino-3-pyridyl)pyrrolidine-2-one (92.2 mg, 520 umol, 1.10 eq), Xantphos (8.21 mg, 14.2 umol, 0.0300 eq), Cs₂CO₃ (308 mg, 946 umol, 2.00 eq) and Pd(OAc)₂ (5.31 mg, 23.7 umol, 0.0500 eq) at 25° C. under N₂ atmosphere. The mixture was stirred at 100° C. for 18 h. LCMS showed 2,5-dichloro-4-(3-phenoxyphenyl)pyrimidine (181-2) was consumed completely and the desired mass was detected. Then H₂O (8 mL) was added to the mixture at 25° C. The reaction mixture was extracted with EtOAc (5 mL*4). The organic layers were dried over anhydrous sodium sulfate, filtered and the filtrate was concentrated under reduced to give a residue. The residue was purified by prep-HPLC (column: Phenomenex Luna C18 75*30 mm*3 um; mobile phase: [water (FA)-ACN]; B %: 20%-60%, 8 min) to give the title compound (Compound 142) (36.0 mg, 77.4 μmol, 16.4% yield, 98.5% purity) as a white solid. LCMS (ES⁺): m/z 458.0 [M+H]⁺.

### Example 182. Synthesis of 1-(5-((5-chloro-4-(1-(cyclopentanecarbonyl)piperidin-3-yl)pyrimidin-2-yl)amino)pyridin-3-yl)pyrrolidin-2-one (Compound 146)

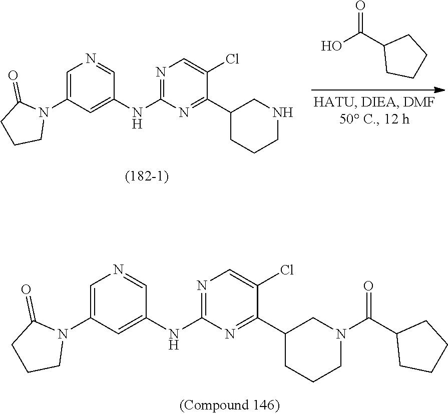 [SMILES: O=C1CCCN1c1cncc(Nc2ncc(Cl)c(C3CCCNC3)n2)c1 ; O=C(O)C1CCCC1 ; O=C(C1CCCC1)N1CCCC(c2nc(Nc3cncc(N4CCCC4=O)c3)ncc2Cl)C1]

Synthesis of 1-(5-((5-chloro-4-(1-(cyclopentanecarbonyl)piperidin-3-yl)pyrimidin-2-yl)amino)pyridin-3-yl)pyrrolidin-2-one (Compound 146). To a solution of cyclopentanecarboxylic acid (42.2 mg, 370 μmol, 40.1 μL, 2.00 eq) in DMF (2 mL) were added DIEA (23.9 mg, 185 μmol, 32.2 μL, 1.00 eq), HATU (84.3 mg, 222 μmol, 1.20 eq) and 1-(5-((5-chloro-4-(piperidin-3-yl)pyrimidin-2-yl)amino)pyridin-3-yl)pyrrolidin-2-one (182-1) (90.0 mg, 185 μmol, 1.00 eq, TFA) at 25° C. The mixture was stirred at 50° C. for 12 h. LCMS showed that the desired mass was detected. The reaction mixture was filtered and the filtrate was concentrated under reduced pressure to give a residue. The residue were purified by prep-HPLC (column: Phenomenex Luna C18 200*40 mm*10 um; mobile phase: [water (FA)-ACN]; B %: 20%-50%, 8 min) to give the title compound (Compound 146) (12.2 mg, 24.4 μmol, 13.2% yield, 93.7% purity) as a white solid. LCMS (ES⁺): m/z 469.1 [M+H]⁺.

### Example 183. Synthesis of 1-(5-((5-chloro-4-(1-(cyclopentanecarbonyl)piperidin-4-yl)pyrimidin-2-yl)amino)pyridin-3-yl)pyrrolidin-2-one (Compound 153)

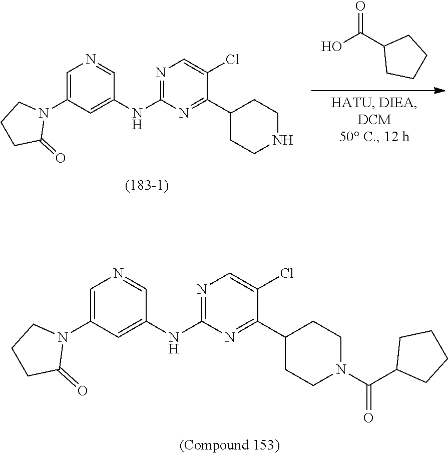 [SMILES: O=C1CCCN1c1cncc(Nc2ncc(Cl)c(C3CCNCC3)n2)c1 ; O=C(O)C1CCCC1 ; O=C(C1CCCC1)N1CCC(c2nc(Nc3cncc(N4CCCC4=O)c3)ncc2Cl)CC1]

Synthesis of 1-(5-((5-chloro-4-(1-(cyclopentanecarbonyl)piperidin-4-yl)pyrimidin-2-yl)amino)pyridin-3-yl)pyrrolidin-2-one (Compound 153). To a solution of 1-[5-[[5-chloro-4-(4-piperidyl)pyrimidin-2-yl]amino]-3-pyridyl]pyrrolidin-2-one (183-1) (200 mg, 536 μmol, 1 eq) in DCM (7 mL) added HATU (306 mg, 805 μmol, 1.50 eq), DIPEA (208 mg, 1.61 mmol, 280 μL, 3.00 eq) at 25° C. After addition, the mixture was stirred at 25° C. for 0.5 h, and then cyclopentanecarboxylic acid (122 mg, 1.07 mmol, 116 μL, 2.00 eq) in DCM (7 mL) was added to the mixture at 25° C. The resulting mixture was stirred at 40° C. for 12 h. LCMS showed that 1-[5-[[5-chloro-4-(4-piperidyl)pyrimidin-2-yl]amino]-3-pyridyl]pyrrolidin-2-one (183-1) was consumed and the desired mass was detected. H₂O (3 mL) was added to the solution, then the mixture was extracted with EtOAc (3 mL*3), the combined organic lays were dried over Na₂SO₄, then concentrated in vacuo to give the crude product. The residue was purified by prep-HPLC (column: Phenomenex Luna C18 75*30 mm*3 um; mobile phase: [water (FA)-ACN]; gradient: 15%-50% B over 8 min) to give the title compound (Compound 153) (5.40 mg, 11.0 μmol, 2.67% yield, 95.2% purity) as a yellow solid. LCMS (ES⁺): m/z 469.3 [M+H]⁺.

### Example 184. Synthesis of 5-(5-fluoro-2-((5-(2-oxopyrrolidin-1-yl)pyridin-3-yl)amino)pyrimidin-4-yl)-1-(oxetan-3-yl)pyridin-2(1H)-one (Compound 155)

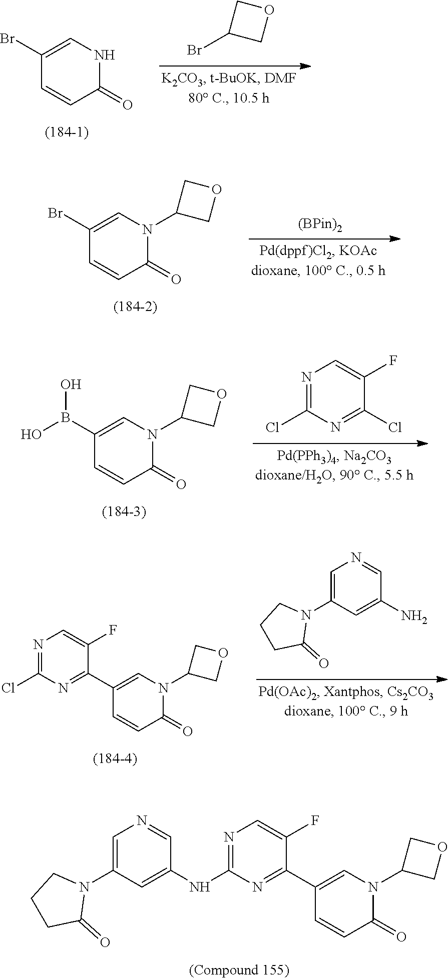

Synthesis of 5-bromo-1-(oxetan-3-yl)pyridin-2(1H)-one (184-2). To a solution of 5-bromopyridin-2(1H)-one (184-1) (6.35 g, 36.5 mmol, 1.00 eq) in DMF (100 mL) were added potassium 2-methylpropan-2-olate (4.10 g, 1.00 eq, 1.00 M, 36.5 mL, 1.00 eq) at 0° C. The mixture was stirred at 25° C. for 30 min. The mixture were added K₂CO₃ (3.53 g, 25.6 mmol, 0.700 eq) and 3-bromooxetane (10.0 g, 73.0 mmol, 2.00 eq) at 25° C. The mixture was stirred at 80° C. for 10 h. LC-MS showed that 5-bromopyridin-2(1H)-one (184-1) was consumed completely and the desired mass was detected. Water (130 mL) was added to the mixture at 25° C. The reaction mixture was extracted with EtOAc (80 mL*4). The organic layers were dried over anhydrous sodium sulfate, filtered and the filtrate was concentrated under reduced to give a residue. The residue was purified by column chromatography (SiO₂, Petroleum ether/Ethyl acetate=1/2 to 1/3) to give the title compound (184-2) (1.35 g, 5.87 mmol, 16.1% yield) as a yellow solid. LCMS (ES⁺): m/z 230.0 [M+H]⁺.

Synthesis of (1-(oxetan-3-yl)-6-oxo-1,6-dihydropyridin-3-yl)boronic acid (184-3). To a solution of 5-bromo-1-(oxetan-3-yl)pyridin-2(1H)-one (184-2) (700 mg, 3.04 mmol, 1.00 eq) in dioxane (17 mL) were added 4,4,5,5-tetramethyl-2-(4,4,5,5-tetramethyl-1,3,2-dioxaborolan-2-yl)-1,3,2-dioxaborolane (1.08 g, 4.26 mmol, 1.40 eq), KOAc (597 mg, 6.09 mmol, 2.00 eq) and Pd(dppf)Cl₂ (111 mg, 152 μmol, 0.0500 eq) at 25° C. under N₂ atmosphere. The mixture was stirred at 100° C. for 0.5 h. LC-MS showed ˜3% of 5-bromo-1-(oxetan-3-yl)pyridin-2(1H)-one (184-2) was remaining, and ˜22% of the desired mass was detected. The reaction mixture was filtered and the filtrate concentrated under reduced pressure to give a residue. The residue was purified by prep-HPLC (column: Phenomenex luna C18 100*40 mm*5 um; mobile phase: [H₂O (0.2% FA)-ACN]; gradient: 20%-60% B over 8.0 min) to give the title compound (184-3) (280 mg, 1.44 mmol, 47.2% yield) as a white solid. LCMS (ES⁺): m/z 196.3 [M+H]⁺.

Synthesis of 5-(2-chloro-5-fluoropyrimidin-4-yl)-1-(oxetan-3-yl)pyridin-2(1H)-one (184-4). To a solution of (1-(oxetan-3-yl)-6-oxo-1,6-dihydropyridin-3-yl)boronic acid (184-3) (200 mg, 1.03 mmol, 1.00 eq) in dioxane (4 mL) and H₂O (0.4 mL) were added 2,4-dichloro-5-fluoropyrimidine (206 mg, 1.23 mmol, 1.20 eq), Na₂CO₃ (217 mg, 2.05 mmol, 2.00 eq) and Pd(PPh₃)₄ (23.7 mg, 20.5 μmol, 0.0200 eq) at 25° C. under N₂ atmosphere. The mixture was stirred at 90° C. for 2 h. LC-MS showed ˜16% of (1-(oxetan-3-yl)-6-oxo-1,6-dihydropyridin-3-yl)boronic acid (184-3) was remaining and it came out on the same peak as the desired mass. The mixture were added 2,4-dichloro-5-fluoropyrimidine (70.0 mg), Na₂CO₃ (70.0 mg) and Pd(PPh₃)₄ (23.7 mg) at 25° C. under N₂ atmosphere. The mixture was stirred at 90° C. for 3.5 h. LC-MS showed (1-(oxetan-3-yl)-6-oxo-1,6-dihydropyridin-3-yl)boronic acid (184-3) was consumed completely and the desired mass was detected. The reaction mixture was filtered with THF (200 mL) and the filtrate concentrated under reduced pressure to give a residue. The residue was purified by prep-HPLC (column: Phenomenex luna C18 100*40 mm*5 um; mobile phase: [H₂O (0.2% FA)-ACN]; gradient: 10%-45% B over 8.0 min) to give the title compound (184-4) (63.0 mg, 179 μmol, 17.4% yield, 80.0% purity) as a white solid. LCMS (ES⁺): m/z 282.0 [M+H]⁺.

Synthesis of 5-(5-fluoro-2-((5-(2-oxopyrrolidin-1-yl)pyridin-3-yl)amino)pyrimidin-4-yl)-1-(oxetan-3-yl)pyridin-2(1H)-one (Compound 155). To a solution of 5-(2-chloro-5-fluoropyrimidin-4-yl)-1-(oxetan-3-yl)pyridin-2(1H)-one (184-4) (60.0 mg, 213 μmol, 1.00 eq) in dioxane (1.2 mL) were added 1-(5-aminopyridin-3-yl)pyrrolidin-2-one (41.5 mg, 234 μmol, 1.10 eq), Xantphos (3.70 mg, 6.39 μmol, 0.0300 eq), Cs₂CO₃ (139 mg, 426 μmol, 2.00 eq) and Pd(OAc)₂ (2.39 mg, 10.7 μmol, 0.0500 eq) at 25° C. under N₂ atmosphere. The mixture was stirred at 100° C. for 9 h. LC-MS showed 5-(2-chloro-5-fluoropyrimidin-4-yl)-1-(oxetan-3-yl)pyridin-2(1H)-one (184-4) was consumed completely and the desired mass was detected. The reaction mixture was filtered and the filtrate concentrated under reduced pressure to give a residue. The residue was purified by prep-HPLC (column: Phenomenex luna C18 100*40 mm*5 um; mobile phase: [H₂O (0.2% FA)-ACN]; gradient: 20%-65% B over 8.0 min) to give the title compound (Compound 155) (14.0 mg, 33.1 μmol, 15.6% yield, 100% purity) as a yellow solid. LCMS (ES⁺): m/z 423.1 [M+H]⁺.

### Example 185. Synthesis of 1-(5-((5-chloro-4-(1-(4-fluorobenzoyl)pyrrolidin-3-yl)pyrimidin-2-yl)amino)pyridin-3-yl)pyrrolidin-2-one (Compound 159)

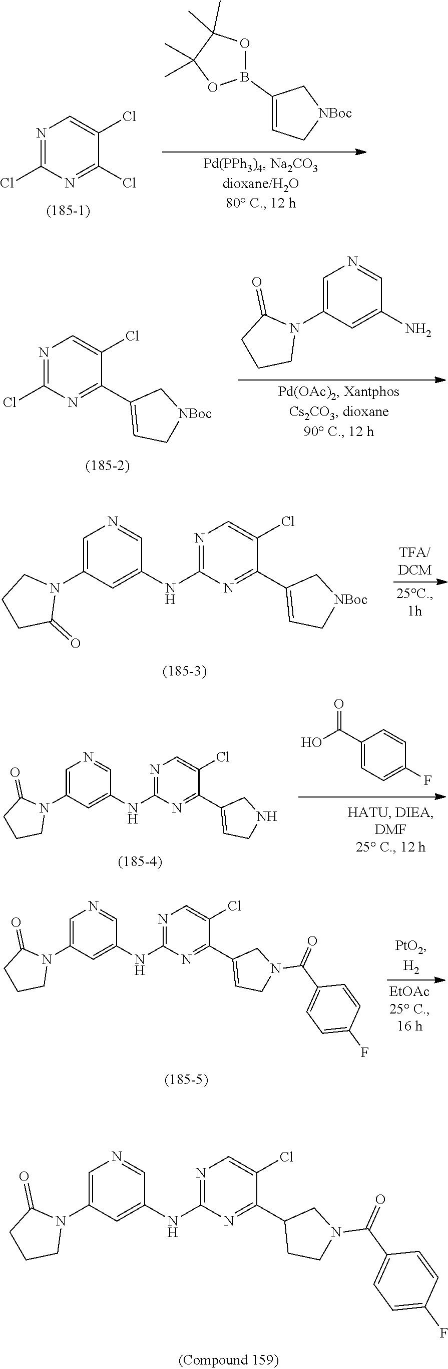

Synthesis of tert-butyl 3-(2,5-dichloropyrimidin-4-yl)-2,5-dihydro-1H-pyrrole-1-carboxylate (185-2). To a solution of tert-butyl 3-(4,4,5,5-tetramethyl-1,3,2-dioxaborolan-2-yl)-2,5-dihydropyrrole-1-carboxylate (10.0 g, 33.9 mmol, 1.00 eq) and 2,4,5-trichloropyrimidine (185-1) (7.46 g, 40.7 mmol, 1.20 eq) in dioxane (100 mL) and H₂O (10 mL) were added Na₂CO₃ (7.18 g, 67.8 mmol, 2.00 eq) and Pd(PPh₃)₄ (1.96 g, 1.69 mmol, 0.0500 eq) at 25° C. under N₂ atmosphere. The mixture was stirred at 80° C. for 12 h under N₂ atmosphere. LCMS showed the desired mass was detected. The mixture was concentrated in vacuo. The mixture was diluted with H₂O (300 mL) and then extracted with EtOAc (70 mL*3). The combined organic layers were dried over Na₂SO₄, filtered and the filtrate was concentrated in vacuo. The residue was purified by column chromatography (SiO₂, Petroleum ether:Ethyl acetate=1:0 to 5:1) to give the title compound (185-2) (7.69 g, 24.3 mmol, 71.8% yield) as a white solid. LCMS (ES⁺): m/z 260.0 [M+H−56]⁺.

Synthesis of tert-butyl 3-(5-chloro-2-((5-(2-oxopyrrolidin-1-yl)pyridin-3-yl)amino)pyrimidin-4-yl)-2,5-dihydro-1H-pyrrole-1-carboxylate (185-3). To a solution of tert-butyl 3-(2,5-dichloropyrimidin-4-yl)-2,5-dihydro-1H-pyrrole-1-carboxylate (185-2) (1.00 g, 3.16 mmol, 1.00 eq) in dioxane (10 mL) were added 1-(5-amino-3-pyridyl)pyrrolidin-2-one (560 mg, 3.16 mmol, 1.00 eq), Cs₂CO₃ (2.06 g, 6.33 mmol, 2.00 eq), Xantphos (54.9 mg, 94.9 μmol, 0.0300 eq) and Pd(OAc)₂ (35.5 mg, 158 μmol, 0.0500 eq) at 25° C. under N₂ atmosphere. The mixture was stirred at 90° C. for 12 h. LCMS showed tert-butyl 3-(2,5-dichloropyrimidin-4-yl)-2,5-dihydro-1H-pyrrole-1-carboxylate (185-2) was consumed completely and the desired mass was detected. Water (9 mL) was added to the mixture at 25° C. Then the reaction mixture was extracted with EtOAc (8 mL*3). The organic layers were dried over anhydrous sodium sulfate, filtered and the filtrate was concentrated under reduced pressure to give a residue. The residue was purified by column chromatography (SiO₂, Ethyl acetate:Methanol=1:0 to 20:1) to give the title compound (185-3) (300 mg, 657 μmol, 20.8% yield) as a yellow solid. LCMS (ES⁺): m/z 457.2 [M+H]⁺.

Synthesis of 1-(5-((5-chloro-4-(2,5-dihydro-1H-pyrrol-3-yl)pyrimidin-2-yl)amino)pyridin-3-yl)pyrrolidin-2-one (185-4). To a solution of tert-butyl 3-(5-chloro-2-((5-(2-oxopyrrolidin-1-yl)pyridin-3-yl)amino)pyrimidin-4-yl)-2,5-dihydro-1H-pyrrole-1-carboxylate (185-3) (300 mg, 657 μmol, 1.00 eq) in DCM (3 mL) was added TFA (1.54 g, 13.5 mmol, 1.00 mL, 20.5 eq) at 25° C. The mixture was stirred at 25° C. for 1 h. LCMS showed tert-butyl 3-(5-chloro-2-((5-(2-oxopyrrolidin-1-yl)pyridin-3-yl)amino)pyrimidin-4-yl)-2,5-dihydro-1H-pyrrole-1-carboxylate (185-3) was consumed completely and the desired mass was detected. The reaction mixture was concentrated under reduced pressure to remove solvent to give the title compound (185-4) (300 mg, crude, TFA) as a yellow oil. LCMS (ES⁺): m/z 357.3 [M+H]⁺.

Synthesis of 1-(5-((5-chloro-4-(1-(4-fluorobenzoyl)-2,5-dihydro-1H-pyrrol-3-yl)pyrimidin-2-yl)amino)pyridin-3-yl)pyrrolidin-2-one (185-5). To a solution of 4-fluorobenzoic acid (80.4 mg, 573 μmol, 1.00 eq) and 1-(5-((5-chloro-4-(2,5-dihydro-1H-pyrrol-3-yl)pyrimidin-2-yl)amino)pyridin-3-yl)pyrrolidin-2-one (185-4) (270 mg, 573 μmol, 1.00 eq, TFA) in DMF (6 mL) were added HATU (327 mg, 860 μmol, 1.50 eq) and DIPEA (222 mg, 1.72 mmol, 300 μL, 3.00 eq) at 25° C. The mixture was stirred at 25° C. for 12 h. LCMS showed 1-(5-((5-chloro-4-(2,5-dihydro-1H-pyrrol-3-yl)pyrimidin-2-yl)amino)pyridin-3-yl)pyrrolidin-2-one (185-4) was consumed completely and the desired mass was detected. Water (5 mL) was added to the mixture at 25° C. Then the reaction mixture was extracted with EtOAc (4 mL*3). The organic layers were dried over anhydrous sodium sulfate, filtered and the filtrate was concentrated under reduced pressure to give a residue. The residue was purified by prep-TLC (SiO₂, Ethyl acetate:Methanol=20:1) to give the title compound (185-5) (205 mg, 428 μmol, 74.7% yield) as a yellow solid. LCMS (ES⁺): m/z 479.3 [M+H]⁺.

Synthesis of 1-(5-((5-chloro-4-(1-(4-fluorobenzoyl)pyrrolidin-3-yl)pyrimidin-2-yl)amino)pyridin-3-yl)pyrrolidin-2-one (Compound 159). To a solution of 1-(5-((5-chloro-4-(1-(4-fluorobenzoyl)-2,5-dihydro-1H-pyrrol-3-yl)pyrimidin-2-yl)amino)pyridin-3-yl)pyrrolidin-2-one (185-5) (100 mg, 209 μmol, 1.00 eq) in EtOAc (4 mL) were added H₂ and PtO₂ (100 mg, 440 μmol, 2.11 eq) at 25° C. The mixture was stirred at 25° C. for 4 h under H₂ (15 psi). LCMS showed 1-(5-((5-chloro-4-(1-(4-fluorobenzoyl)-2,5-dihydro-1H-pyrrol-3-yl)pyrimidin-2-yl)amino)pyridin-3-yl)pyrrolidin-2-one (185-5) was not consumed completely and the desired mass was detected. The mixture was stirred at 25° C. for 12 h under H₂ (15 psi). LCMS showed 1-(5-((5-chloro-4-(1-(4-fluorobenzoyl)-2,5-dihydro-1H-pyrrol-3-yl)pyrimidin-2-yl)amino)pyridin-3-yl)pyrrolidin-2-one (185-5) was not consumed completely and the desired mass was detected. The reaction mixture was filtered and the filtrate was concentrated under reduced pressure to give a residue. The residue were purified by prep-HPLC (column: Phenomenex Luna C18 100*30 mm*3 um; mobile phase: [H₂O (0.2% FA)-ACN]; gradient: 5%-45% B over 8.0 min) to give the title compound (Compound 159) (11.2 mg, 22.1 μmol, 10.6% yield, 94.9% purity) as a white solid. LCMS (ES⁺): m/z 481.0 [M+H]⁺.

### Example 186. 1-(5-((5-chloro-4-(1-(oxetane-3-carbonyl)piperidin-3-yl)pyrimidin-2-yl)amino)pyridin-3-yl)pyrrolidin-2-one (Compound 160)

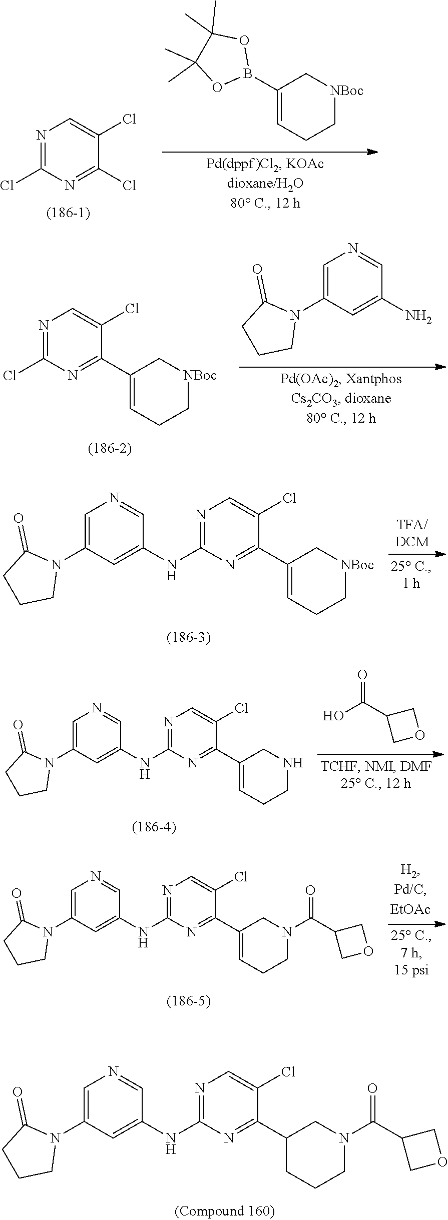

Synthesis of tert-butyl 5-(2,5-dichloropyrimidin-4-yl)-3,6-dihydropyridine-1(2H)-carboxylate (186-2). Two batches in parallel were set up. A mixture of 2,4,5-trichloropyrimidine (186-1) (5.00 g, 16.2 mmol, 1.00 eq), tert-butyl 5-(4,4,5,5-tetramethyl-1,3,2-dioxaborolan-2-yl)-3,6-dihydropyridine-1(2H)-carboxylate (2.97 g, 16.2 mmol, 1.00 eq), Pd(dppf)Cl₂ (1.18 g, 1.62 mmol, 0.100 eq), KOAc (4.76 g, 48.5 mmol, 3.00 eq) in dioxane (60 mL) and H₂O (15 mL) was degassed and purged with N₂ for 3 times at 25° C., and then the mixture was stirred at 80° C. for 12 h under N₂ atmosphere. LCMS showed the desired mass was detected. Two batches in parallel were combined. The reaction mixture was diluted with H₂O (150 mL) and extracted with EtOAc (150 mL*3). The combined organic layers dried over Na₂SO₄, filtered and concentrated under reduced pressure. The residue was purified by column chromatography (SiO₂, PE/EtOAc=1/0 to 1/1) to give the title compound (186-2) (17.8 g, crude) as a yellow solid. LCMS (ES⁺): m/z 274.1 [M+H−56]⁺.

Synthesis of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (186-3). A mixture of tert-butyl 5-(2,5-dichloropyrimidin-4-yl)-3,6-dihydropyridine-1(2H)-carboxylate (186-2) (10.0 g, 30.3 mmol, 1.00 eq), 1-(5-aminopyridin-3-yl)pyrrolidin-2-one (5.37 g, 30.3 mmol, 1.00 eq), Pd(OAc)₂ (680 mg, 3.03 mmol, 0.100 eq), Xantphos (1.75 g, 3.03 mmol, 0.100 eq) and Cs₂CO₃ (29.6 g, 90.9 mmol, 3.00 eq) in dioxane (100 mL) was degassed and purged with N₂ for 3 times at 25° C., and then the mixture was stirred at 80° C. for 12 h under N₂ atmosphere. LCMS showed tert-butyl 5-(2,5-dichloropyrimidin-4-yl)-3,6-dihydropyridine-1(2H)-carboxylate (186-2) was consumed completely and desired mass was detected. The reaction mixture was diluted with H₂O (100 mL) and extracted with EtOAc (100 mL*3). The combined organic layers were dried anhydrous Na₂SO₄, filtered and concentrated under reduced pressure. The residue was purified by column chromatography (SiO₂, PE/EtOAc=1/0 to 1/1) to give the title compound (186-3) (12.5 g, 20.8 mmol, 68.5% yield, 78.5% purity) as a yellow solid. LCMS (ES⁺): m/z 471.2 [M+H]⁺.

Synthesis of 1-(5-((5-chloro-4-(1,2,5,6-tetrahydropyridin-3-yl)pyrimidin-2-yl)amino)pyridin-3-yl)pyrrolidin-2-one (186-4). To a solution of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (186-3) (500 mg, 1.06 mmol, 1.00 eq) in DCM (6 mL) was added TFA (2 mL) at 25° C., the mixture was stirred at 25° C. for 1 h. LCMS showed tert-butyl 5-(5-chloro-2-((5-(2-oxopyrrolidin-1-yl)pyridin-3-yl)amino)pyrimidin-4-yl)-3,6-dihydropyridine-1(2H)-carboxylate (186-3) was consumed completely and the desired mass was detected. The mixture was concentrated under reduced pressure to give the title compound (186-4) (500 mg, crude, TFA) as a yellow oil. LCMS (ES⁺): m/z 371.2 [M+H]⁺.

Synthesis of 1-(5-((5-chloro-4-(1-(oxetane-3-carbonyl)-1,2,5,6-tetrahydropyridin-3-yl)pyrimidin-2-yl)amino)pyridin-3-yl)pyrrolidin-2-one (186-5). To a solution of oxetane-3-carboxylic acid (73.7 mg, 722 μmol, 1.00 eq) in DMF (5.00 mL) were added TCFH (243 mg, 866 μmol, 1.20 eq) and NMI (178 mg, 2.17 mmol, 173 μL, 3.00 eq) at 25° C., then 1-(5-((5-chloro-4-(1,2,5,6-tetrahydropyridin-3-yl)pyrimidin-2-yl)amino)pyridin-3-yl)pyrrolidin-2-one (186-4) (350 mg, 722 μmol, 1.00 eq, TFA) was added to the mixture, the mixture was stirred at 25° C. for 12 h. LCMS showed 1-(5-((5-chloro-4-(1,2,5,6-tetrahydropyridin-3-yl)pyrimidin-2-yl)amino)pyridin-3-yl)pyrrolidin-2-one (186-4) was consumed completely and the desired mass was detected. H₂O (10 mL) was added to the solution, the mixture was extracted with EtOAc (5 mL*3), then the combined organic layers were washed with brine (5 mL*3), the organic layer was dried over Na₂SO₄, then concentrated under reduced pressure. The mixture was purified by prep-TLC (SiO₂, EtOAc/MeOH=5/1) to give the title compound (186-5) (310 mg, 681 μmol, 94.4% yield) as a yellow oil. LCMS (ES⁺): m/z 455.2 [M+H]⁺.

Synthesis of 1-(5-((5-chloro-4-(1-(oxetane-3-carbonyl)piperidin-3-yl)pyrimidin-2-yl)amino)pyridin-3-yl)pyrrolidin-2-one (Compound 160). To a solution of 1-(5-((5-chloro-4-(1-(oxetane-3-carbonyl)-1,2,5,6-tetrahydropyridin-3-yl)pyrimidin-2-yl)amino)pyridin-3-yl)pyrrolidin-2-one (186-5) (310 mg, 681 μmol, 1.00 eq) in EtOAc (10.0 mL) was added 10% Pd/C (620 mg, 50% purity) at 25° C. under N₂. The mixture was stirred at 25° C. under H₂ (15 PSI) for 4 h. LCMS showed 1-(5-((5-chloro-4-(1-(oxetane-3-carbonyl)-1,2,5,6-tetrahydropyridin-3-yl)pyrimidin-2-yl)amino)pyridin-3-yl)pyrrolidin-2-one (186-5) remained partly and the desired mass was detected, then 10% Pd/C (310 mg, 50% purity) was added to the mixture, the mixture was stirred at 25° C. under H₂ (15 PSI) for 3 h. LCMS showed 1-(5-((5-chloro-4-(1-(oxetane-3-carbonyl)-1,2,5,6-tetrahydropyridin-3-yl)pyrimidin-2-yl)amino)pyridin-3-yl)pyrrolidin-2-one (186-5) was consumed completely and the desired mass was detected. The mixture was filtered, the filtrate was concentrate under reduced pressure. The residue was purified by neutral prep-HPLC (column: Waters Xbridge BEH C18 100*25 mm*10 um; mobile phase: [H₂O (10 mM NH₄HCO₃)-ACN]; gradient: 20%-50% B over 8.0 min) to give the title compound (Compound 160) (31.5 mg, 68.2 μmol, 10.0% yield, 99.0% purity) as a white solid. LCMS (ES⁺): m/z 457.2 [M+H].

### Example 187. Synthesis of 1-(5-((5-chloro-4-(1-(4-fluorobenzoyl)piperidin-3-yl)pyrimidin-2-yl)amino)pyridin-3-yl)pyrrolidin-2-one (Compound 162)

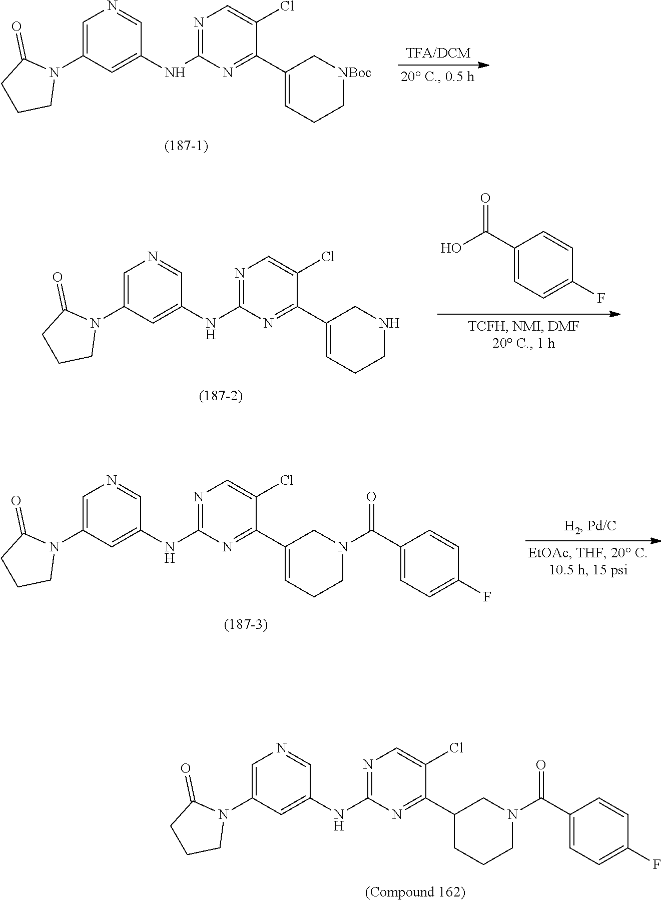 [SMILES: CC(C)(C)OC(=O)N1CCC=C(c2nc(Nc3cncc(N4CCCC4=O)c3)ncc2Cl)C1 ; O=C1CCCN1c1cncc(Nc2ncc(Cl)c(C3=CCCNC3)n2)c1 ; O=C(O)c1ccc(F)cc1 ; O=C(c1ccc(F)cc1)N1CCC=C(c2nc(Nc3cncc(N4CCCC4=O)c3)ncc2Cl)C1 ; O=C(c1ccc(F)cc1)N1CCCC(c2nc(Nc3cncc(N4CCCC4=O)c3)ncc2Cl)C1]

Synthesis of 1-(5-((5-chloro-4-(1,2,5,6-tetrahydropyridin-3-yl)pyrimidin-2-yl)amino)pyridin-3-yl)pyrrolidin-2-one (187-2). A solution of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (187-1) (500 mg, 1.06 mmol, 1.00 eq) in DCM (6 mL) and TFA (2 mL) was stirred at 20° C. for 0.5 h. LCMS showed tert-butyl 5-(5-chloro-2-((5-(2-oxopyrrolidin-1-yl)pyridin-3-yl)amino)pyrimidin-4-yl)-3,6-dihydropyridine-1(2H)-carboxylate (187-1) was consumed, the desired target MS was detected. The mixture was concentrated in vacuo to give the title compound (187-2) (510 mg, crude, TFA) as a yellow oil. LCMS (ES⁺): m/z 371.1 [M+H]⁺.

Synthesis of 1-(5-((5-chloro-4-(1-(4-fluorobenzoyl)-1,2,5,6-tetrahydropyridin-3-yl)pyrimidin-2-yl)amino)pyridin-3-yl)pyrrolidin-2-one (187-3). To a solution of 1-(5-((5-chloro-4-(1,2,5,6-tetrahydropyridin-3-yl)pyrimidin-2-yl)amino)pyridin-3-yl)pyrrolidin-2-one (187-2) (450 mg, 928 μmol, 1.00 eq, TFA) and 4-fluorobenzoic acid (143 mg, 1.02 mmol, 1.10 eq) in DMF (3 mL) was added TCFH (521 mg, 1.86 mmol, 2.00 eq) and NMI (228.6 mg, 2.78 mmol, 222 μL, 3.00 eq) at 20° C., then the mixture was stirred at 20° C. for 1 h. LCMS showed 1-(5-((5-chloro-4-(1,2,5,6-tetrahydropyridin-3-yl)pyrimidin-2-yl)amino)pyridin-3-yl)pyrrolidin-2-one (187-2) was consumed, desired target MS was detected. The mixture was concentrated in vacuo, H₂O (3 mL) was added to the mixture and the mixture was extracted with EtOAc (3 mL*3), the combined organic layers were washed with saturated aqueous NaCl solution (3 mL*3), dried over Na₂SO₄, filtered and the filtrate was concentrated in vacuo to give a residue. The residue was purified by prep-TLC (SiO₂, EtoAC/MeOH=10/1) to give the title compound (187-3) (400 mg, 811 μmol, 87.4% yield) as a green solid. LCMS (ES⁺): m/z 493.3 [M+H]⁺.

Synthesis of 1-(5-((5-chloro-4-(1-(4-fluorobenzoyl)piperidin-3-yl)pyrimidin-2-yl)amino)pyridin-3-yl)pyrrolidin-2-one (Compound 162) To a mixture of 1-(5-((5-chloro-4-(1-(4-fluorobenzoyl)-1,2,5,6-tetrahydropyridin-3-yl)pyrimidin-2-yl)amino)pyridin-3-yl)pyrrolidin-2-one (187-3) (350 mg, 710 μmol, 1.00 eq) in EtOAc (1 mL) and THF (1 mL) was added 10% Pd/C (50 mg, 50% purity) at 20° C., then the mixture was stirred at 20° C. for 0.5 h under H₂ at 15 Psi. LCMS showed 1-(5-((5-chloro-4-(1-(4-fluorobenzoyl)-1,2,5,6-tetrahydropyridin-3-yl)pyrimidin-2-yl)amino)pyridin-3-yl)pyrrolidin-2-one (187-3) was remained, desired target MS was detected, then the mixture was stirred at 20° C. for 10 h under H₂ at 15 Psi. LCMS showed 1-(5-((5-chloro-4-(1-(4-fluorobenzoyl)-1,2,5,6-tetrahydropyridin-3-yl)pyrimidin-2-yl)amino)pyridin-3-yl)pyrrolidin-2-one (187-3) was consumed, desired target MS was detected The mixture was filtered and the filtrate was concentrated under reduced pressure to give a residue. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 162) (18.0 mg, 36.1 μmol, 5.09% yield, 99.3% purity) as a white solid. LCMS (ES⁺): m/z 495.1 [M+H]⁺.

### Example 188. Synthesis of 5-[3-[5-chloro-2-[[5-(2-oxopyrrolidin-1-yl)-3-pyridyl]amino]pyrimidin-4-yl]piperidine-1-carbonyl]-1H-pyridin-2-one (Compound 163)

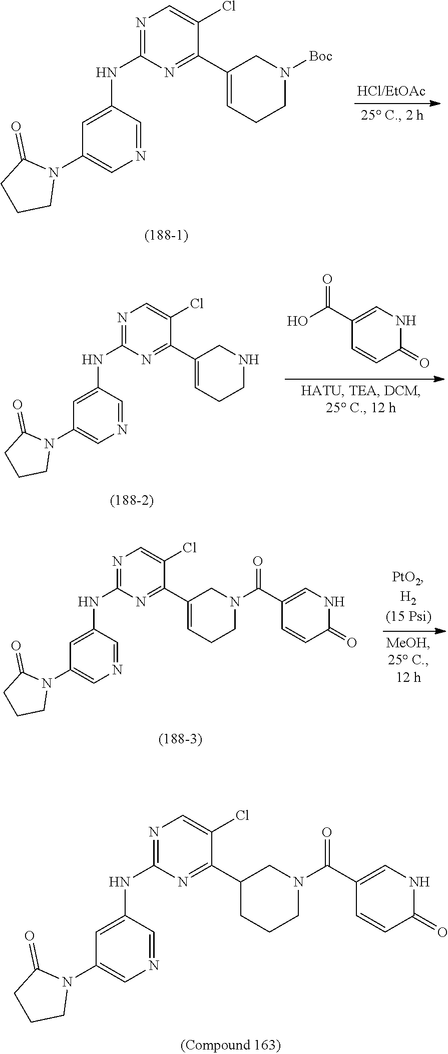 [SMILES: CC(C)(C)OC(=O)N1CCC=C(c2nc(Nc3cncc(N4CCCC4=O)c3)ncc2Cl)C1 ; O=C1CCCN1c1cncc(Nc2ncc(Cl)c(C3=CCCNC3)n2)c1 ; O=C(O)c1ccc(=O)[nH]c1 ; O=C(c1ccc(=O)[nH]c1)N1CCC=C(c2nc(Nc3cncc(N4CCCC4=O)c3)ncc2Cl)C1 ; O=C(c1ccc(=O)[nH]c1)N1CCCC(c2nc(Nc3cncc(N4CCCC4=O)c3)ncc2Cl)C1]

Synthesis of 1-(5-((5-chloro-4-(1,2,5,6-tetrahydropyridin-3-yl)pyrimidin-2-yl)amino)pyridin-3-yl)pyrrolidin-2-one (188-2). To a solution of tert-butyl 5-[5-chloro-2-[[5-(2-oxopyrrolidin-1-yl)-3-pyridyl]amino]pyrimidin-4-yl]-3,6-dihydro-2H-pyridine-1-carboxylate (188-1) (500 mg, 1.06 mmol, 1.00 eq) in DCM (4 mL) was added TFA (1 mL) at 25° C., the mixture was stirred at 25° C. for 1 h. LCMS showed tert-butyl 5-[5-chloro-2-[[5-(2-oxopyrrolidin-1-yl)-3-pyridyl]amino]pyrimidin-4-yl]-3,6-dihydro-2H-pyridine-1-carboxylate (188-1) was consumed and the desired mass was detected. The mixture was concentrated in reduced pressure to give title compound (188-2) (510 mg, crude, TFA) as brown oil liquid. LCMS (ES⁺): m/z 371.2 [M+H]⁺.

Synthesis of 5-[5-[5-chloro-2-[[5-(2-oxopyrrolidin-1-yl)-3-pyridyl]amino]pyrimidin-4-yl]-3,6-dihydro-2H-pyridine-1-carbonyl]-1H-pyridin-2-one (188-3). To a solution of 6-oxo-1H-pyridine-3-carboxylic acid (146 mg, 1.05 mmol, 1.00 eq) in DMF (5 mL) was added NMI (259 mg, 3.16 mmol, 251 μL, 3.00 eq) to adjust pH to 8 at 25° C., then TCFH (590 mg, 2.10 mmol, 2.00 eq) was added at 25° C., the mixture was stirred at 25° C. for 15 min. 1-[5-[[5-chloro-4-(1,2,3,6-tetrahydropyridin-5-yl)pyrimidin-2-yl]amino]-3-pyridyl]pyrrolidin-2-one (510 mg, 1.05 mmol, 1.00 eq, TFA) (188-2) was added at 25° C., the mixture was stirred at 60° C. for 1 h. LCMS showed that the 1-[5-[[5-chloro-4-(1,2,3,6-tetrahydropyridin-5-yl)pyrimidin-2-yl]amino]-3-pyridyl]pyrrolidin-2-one (188-2) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in reduced pressure to give the crude product. The mixture was purified by prep-TLC (SiO₂, PE/EtOAc=5/1) to give title compound (188-3) (300 mg, 610 μmol, 58.0% yield) as white solid. LCMS (ES⁺): m/z 492.3 [M+H]⁺.

Synthesis of 5-[3-[5-chloro-2-[[5-(2-oxopyrrolidin-1-yl)-3-pyridyl]amino]pyrimidin-4-yl]piperidine-1-carbonyl]-1H-pyridin-2-one (Compound 163). To a solution of 5-[5-[5-chloro-2-[[5-(2-oxopyrrolidin-1-yl)-3-pyridyl]amino]pyrimidin-4-yl]-3,6-dihydro-2H-pyridine-1-carbonyl]-1H-pyridin-2-one (188-3) (300 mg, 610 μmol, 1.00 eq) in MeOH (20 mL) was added 10% Pd/C (400 mg, 50% purity) at 25° C., the mixture was stirred at 25° C. for 4 h under H₂ (15 psi). LCMS showed 5-[5-[5-chloro-2-[[5-(2-oxopyrrolidin-1-yl)-3-pyridyl]amino]pyrimidin-4-yl]-3,6-dihydro-2H-pyridine-1-carbonyl]-1H-pyridin-2-one (188-3) was consumed and the desired mass was detected. The mixture was filtered and the filtrate was concentrated in reduced pressure. This mixture was purified by acidic prep-HPLC (FA) (column: Phenomenex Luna C18 100*30 mm*3 um; mobile phase: [H₂O (0.2% FA)-ACN]; gradient: 1%-40% B over 8.0 min) to give title compound (Compound 163) (4.40 mg, 8.91 μmol, 1.46% yield, 100% purity). LCMS (ES⁺): m/z 494.0 [M+H]⁺.

### Example 189. Synthesis of 3-(3-(5-chloro-2-((5-(2-oxopyrrolidin-1-yl)pyridin-3-yl)amino)pyrimidin-4-yl)piperidine-1-carbonyl)pyridin-2(1H)-one (Compound 165)

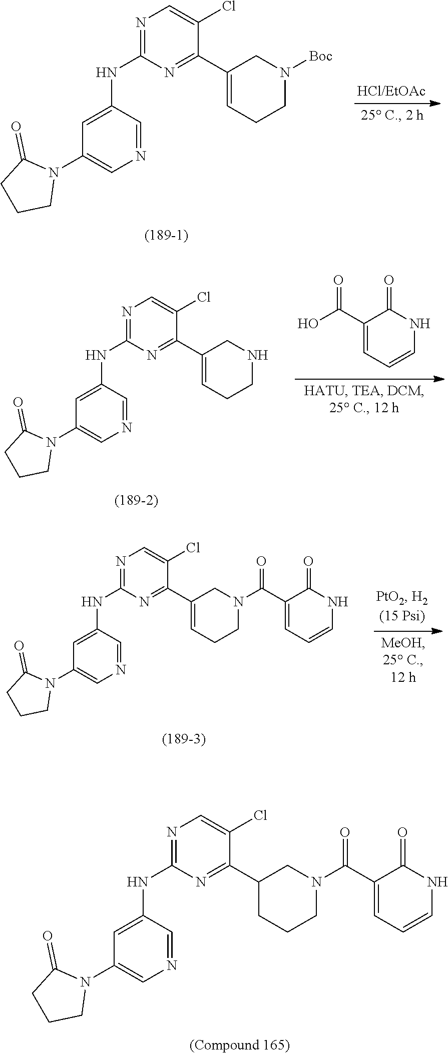 [SMILES: CC(C)(C)OC(=O)N1CCC=C(c2nc(Nc3cncc(N4CCCC4=O)c3)ncc2Cl)C1 ; O=C1CCCN1c1cncc(Nc2ncc(Cl)c(C3=CCCNC3)n2)c1 ; O=C(O)c1ccc[nH]c1=O ; O=C(c1ccc[nH]c1=O)N1CCC=C(c2nc(Nc3cncc(N4CCCC4=O)c3)ncc2Cl)C1 ; O=C(c1ccc[nH]c1=O)N1CCCC(c2nc(Nc3cncc(N4CCCC4=O)c3)ncc2Cl)C1]

Synthesis of 1-[5-[[5-chloro-4-(1,2,3,6-tetrahydropyridin-5-yl)pyrimidin-2-yl]amino]-3-pyridyl]pyrrolidin-2-one (189-2). To a solution of tert-butyl 5-[5-chloro-2-[[5-(2-oxopyrrolidin-1-yl)-3-pyridyl]amino]pyrimidin-4-yl]-3,6-dihydro-2H-pyridine-1-carboxylate (189-1) (2.00 g, 4.25 mmol, 1.00 eq) was added HCl/EtOAc (4.00 M, 15.0 mL, 14.1 eq) at 25° C. The mixture was stirred at 25° C. for 2 h. LC-MS showed tert-butyl 5-[5-chloro-2-[[5-(2-oxopyrrolidin-1-yl)-3-pyridyl]amino]pyrimidin-4-yl]-3,6-dihydro-2H-pyridine-1-carboxylate (189-1) was consumed completely and the desired mass was detected. The reaction mixture was concentrated under reduced pressure to give the title compound (189-2) (2.10 g, crude, HCl) as a brown oil. LCMS (ES⁺): m/z 371.1 [M+H]⁺.

Synthesis of 3-[5-[5-chloro-2-[[5-(2-oxopyrrolidin-1-yl)-3-pyridyl]amino]pyrimidin-4-yl]-3,6-dihydro-2H-pyridine-1-carbonyl]-1H-pyridin-2-one (189-3). To a solution of 1-[5-[[5-chloro-4-(1,2,3,6-tetrahydropyridin-5-yl)pyrimidin-2-yl]amino]-3-pyridyl]pyrrolidin-2-one (189-2) (500 mg, 1.23 mmol, 1.00 eq, HCl) in DCM (15.0 mL) was added HATU (256 mg, 674 μmol, 0.500 eq), 2-oxo-1H-pyridine-3-carboxylic acid (225 mg, 1.62 mmol, 1.20 eq) and TEA (204 mg, 2.02 mmol, 281 μL, 1.50 eq) at 25° C. The mixture was stirred at 25° C. for 12 h. LC-MS showed 1-[5-[[5-chloro-4-(1,2,3,6-tetrahydropyridin-5-yl)pyrimidin-2-yl]amino]-3-pyridyl]pyrrolidin-2-one (189-2) was consumed completely and the desired mass was detected. The reaction diluted with H₂O 20.0 mL and combined organic layers were extracted with EtOAc (50.0 mL*3) concentrated under reduced pressure. The residue was purified by column chromatography (SiO₂, DCM:MeOH=1:0 to 5:1) to give the title compound (189-3) (1.20 g, 2.37 mmol, 43.8% yield, 97.0% purity) as a brown solid. LCMS (ES⁺): m/z 492.2 [M+H]⁺.

Synthesis of 3-(3-(5-chloro-2-((5-(2-oxopyrrolidin-1-yl)pyridin-3-yl)amino)pyrimidin-4-yl)piperidine-1-carbonyl)pyridin-2(1H)-one (Compound 165). The reaction was set up in two parallel batches. A mixture of 3-[5-[5-chloro-2-[[5-(2-oxopyrrolidin-1-yl)-3-pyridyl]amino]pyrimidin-4-yl]-3,6-dihydro-2H-pyridine-1-carbonyl]-1H-pyridin-2-one (189-3) (200 mg, 406 μmol, 1.00 eq) and PtO₂ (138 mg, 609 μmol, 1.50 eq) in MeOH (10.0 mL) at 25° C. was degassed and purged with H₂ for 3 times, and then the mixture was stirred at 25° C. for 12 h under H₂ (15 psi) atmosphere. One batch in parallel was monitored in random, LC-MS showed 3-[5-[5-chloro-2-[[5-(2-oxopyrrolidin-1-yl)-3-pyridyl]amino]pyrimidin-4-yl]-3,6-dihydro-2H-pyridine-1-carbonyl]-1H-pyridin-2-one (189-3) was consumed completely and the desired mass was detected. The two batches were combined. The reaction concentrated under reduced pressure. The residue was purified by prep-HPLC (column: Phenomenex luna C18 100*40 mm*5 um; mobile phase: [H₂O (0.2% FA)-ACN]; gradient: 1%-40% B over 8.0 min) to give the title compound (Compound 165) (32.9 mg, 63.6 μmol, 15.6% yield, 95.6% purity) as a white solid. LCMS (ES⁺): m/z 494.1 [M+H]⁺.

### Example 190. Synthesis of 1-cyclobutyl-5-(5-fluoro-2-((5-(2-oxopyrrolidin-1-yl)pyridin-3-yl)amino)pyrimidin-4-yl)pyridin-2(1H)-one (Compound 157) and 1-(cyclopropylmethyl)-5-(5-fluoro-2-((5-(2-oxopyrrolidin-1-yl)pyridin-3-yl)amino)pyrimidin-4-yl)pyridin-2(1H)-one (Compound 158)

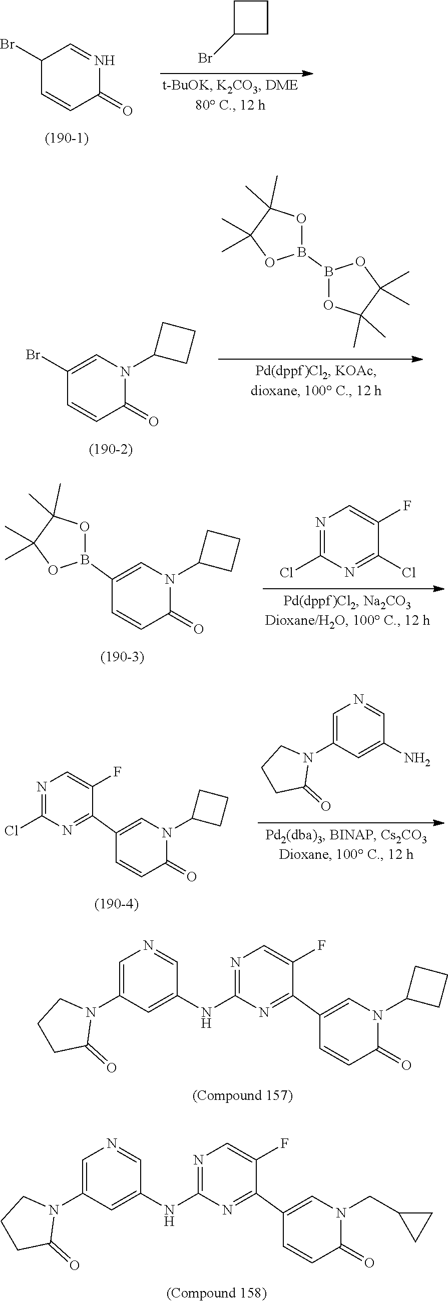

Synthesis of 5-bromo-1-cyclobutylpyridin-2(1H)-one (190-2). To a solution of 5-bromopyridin-2(1H)-one (190-1) (5.00 g, 28.7 mmol, 1.00 eq) and bromocyclobutane (7.76 g, 57.4 mmol, 5.41 mL, 2.00 eq) in DME (100 mL) was added K₂CO₃ (2.78 g, 20.1 mmol, 0.700 eq) and t-BuOK (9.67 g, 86.2 mmol, 3.00 eq) at 25° C. The mixture was stirred at 80° C. for 12 h. LC-MS showed desired mass was detected. The reaction mixture was quenched by addition water 50 mL at 0° C., and extracted with EtOAc (50 mL*3), the combined organic layers were washed with brine (50 mL*3), dried over Na₂SO₄, filtered and concentrated under reduced pressure. The residue was purified by column chromatography (SiO₂, Petroleum ether/Ethyl acetate=1:0 to 1:1) to give the title compound (190-2) (1.96 g, 8.59 mmol, 39.2% yield) as a yellow oil. LCMS (ES⁺): m/z 227.9, 229.9 [M+H]⁺.

Synthesis of 1-cyclobutyl-5-(4,4,5,5-tetramethyl-1,3,2-dioxaborolan-2-yl)pyridin-2(1H)-one (190-3). To a solution of 5-bromo-1-cyclobutylpyridin-2(1H)-one (190-2) (650 mg, 2.85 mmol, 1.00 eq) and 4,4,4′4′,5,5,5′,5′-octamethyl-2,2′-bi(1,3,2-dioxaborolane) (796 mg, 3.13 mmol, 1.10 eq) in dioxane (30 mL) was added KOAc (839 mg, 8.55 mmol, 3.00 eq) at 20° C. Then Pd(dppf)Cl₂ (417 mg, 569 μmol, 0.200 eq) was added to the mixture under N₂. The mixture was stirred at 100° C. for 12 h. LC-MS showed the desired mass was detected. The reaction mixture was diluted with H₂O 50 mL and extracted with EtOAc 40 mL*3. The combined organic layers were dried over Na₂SO₄, filtered and concentrated under reduced pressure. The residue was purified by column chromatography (SiO₂, Petroleum ether/Ethyl acetate=1:0 to 1:1) to give the title compound (190-3) (1.9 g, crude) as a yellow oil. LCMS (ES⁺): m/z 276.2 [M+H]⁺.

Synthesis of 5-(2-chloro-5-fluoro-pyrimidin-4-yl)-1-cyclobutyl-pyridin-2-one (190-4). To a solution of 1-cyclobutyl-5-(4,4,5,5-tetramethyl-1,3,2-dioxaborolan-2-yl)pyridin-2(1H)-one (190-3) (900 mg, 3.27 mmol, 1.00 eq) and 2,4-dichloro-5-fluoropyrimidine (600 mg, 3.60 mmol, 1.10 eq) in dioxane (10 mL) and H₂O (2 mL) was added Na₂CO₃ (1.04 g, 9.81 mmol, 3.00 eq), Pd(dppf)Cl₂ (239 mg, 327 μmol, 0.100 eq) at 20° C. under N₂. The mixture was stirred at 100° C. for 12 h. LC-MS showed the desired mass was detected. The reaction mixture was diluted with H₂O 10 mL and extracted with EtOAc 40 mL*3. The combined organic layers were dried over Na₂SO₄, filtered and concentrated under reduced pressure. The residue was purified by column chromatography (SiO₂, Petroleum ether:Ethyl acetate=1:0 to 0:1) to give the title compound (190-4) (650 mg, crude) as a white solid. LCMS (ES⁺): m/z 280.1 [M+H]⁺.

Synthesis of 1-cyclobutyl-5-(5-fluoro-2-((5-(2-oxopyrrolidin-1-yl)pyridin-3-yl)amino)pyrimidin-4-yl)pyridin-2(1H)-one (Compound 157) and 1-(cyclopropylmethyl)-5-(5-fluoro-2-((5-(2-oxopyrrolidin-1-yl)pyridin-3-yl)amino)pyrimidin-4-yl)pyridin-2(1H)-one (Compound 158). To a solution of 5-(2-chloro-5-fluoro-pyrimidin-4-yl)-1-cyclobutyl-pyridin-2-one (190-4) (300 mg, 1.07 mmol, 1.00 eq) and 1-(5-amino-3-pyridyl)pyrrolidin-2-one (209 mg, 1.18 mmol, 1.10 eq) in dioxane (10 mL) was added BINAP (133 mg, 214 μmol, 0.200 eq), Cs₂CO₃ (1.05 g, 3.22 mmol, 3.00 eq) and Pd₂(dba)₃ (98.2 mg, 107 μmol, 0.100 eq) at 20° C. under N₂. The mixture was stirred at 100° C. for 12 h. LC-MS showed the desired mass was detected. The reaction mixture was filtered and the filtrate was concentrated under reduced pressure. The residue was purified by prep-HPLC (column: Phenomenex luna C18 (250*70 mm, 15 um); mobile phase: [H₂O (0.2% FA)-ACN]; gradient: 25%-55% B over 20.0 min) to give the crude compound, the crude compound was further separated by SFC (SFC: column: DAICEL CHIRALPAK AD (250 mm*30 mm, 10 um); mobile phase: [CO₂-EtOH (0.1% NH₃H₂O)]; B %: 60%, isocratic elution mode) to give the title compound (Compound 157) (24.8 mg, 56.7 μmol, 2.64% yield, 96.1% purity) as a white solid. LCMS (ES⁺): m/z 421.0 [M+H]⁺ and the title compound (Compound 158) (24.8 mg, 56.7 μmol, 2.64% yield, 96.1% purity) as a white solid. LCMS (ES⁺): m/z 421.0 [M+H]⁺.

### Example 191. Synthesis of 6-(3-(5-chloro-2-((5-(2-oxopyrrolidin-1-yl)pyridin-3-yl)amino)pyrimidin-4-yl)piperidine-1-carbonyl)pyridin-2(1H)-one (Compound 161)

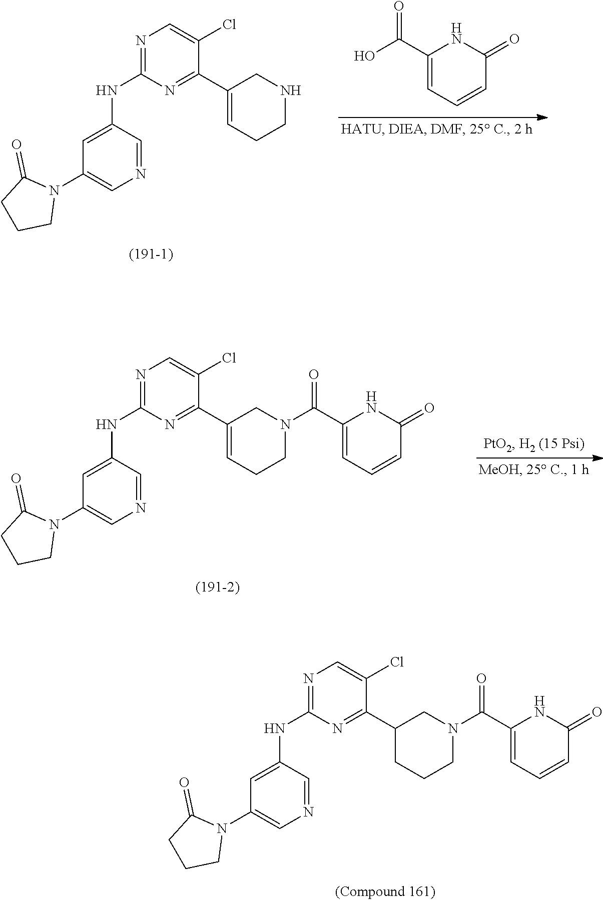 [SMILES: O=C1CCCN1c1cncc(Nc2ncc(Cl)c(C3=CCCNC3)n2)c1 ; O=C(O)c1cccc(=O)[nH]1 ; O=C(c1cccc(=O)[nH]1)N1CCC=C(c2nc(Nc3cncc(N4CCCC4=O)c3)ncc2Cl)C1 ; O=C(c1cccc(=O)[nH]1)N1CCCC(c2nc(Nc3cncc(N4CCCC4=O)c3)ncc2Cl)C1]

Synthesis of 6-(5-(5-chloro-2-((5-(2-oxopyrrolidin-1-yl)pyridin-3-yl)amino)pyrimidin-4-yl)-1,2,3,6-tetrahydropyridine-1-carbonyl)pyridin-2(1H)-one (191-2). To a solution of 6-oxo-1H-pyridine-2-carboxylic acid (450 mg, 3.24 mmol, 1.20 eq) in DMF (5 mL) was added DIEA (1.39 g, 10.7 mmol, 1.88 mL, 4.00 eq) and HATU (1.08 g, 2.83 mmol, 1.05 eq) at 25° C. The mixture was stirred at 25° C. for 0.5 h. To a solution of 1-(5-((5-chloro-4-(1,2,5,6-tetrahydropyridin-3-yl)pyrimidin-2-yl)amino)pyridin-3-yl)pyrrolidin-2-one (191-1) (1.00 g, 2.70 mmol, 1.00 eq) in DMF (10 mL) was added DIEA (348 mg, 2.70 mmol, 469 μL, 1.00 eq) until pH ˜7. Then the two solutions were mixed together and the mixture was stirred at 25° C. for 1.5 h. LC-MS showed the desired mass was detected. The reaction mixture was diluted with water 10 mL and extracted with DCM (20 mL*3). The combined organic layers were washed with brine (50 mL*3), dried over Na₂SO₄, filtered and concentrated under reduced pressure. The residue was purified by column chromatography (SiO₂, Dichloromethane:Methanol=1:0 to 6:1) to give the title compound (191-2) (180 mg, 329 μmol, 12.2% yield, 90.0% purity) as a yellow solid. LCMS (ES⁺): m/z 492.1 [M+H]⁺.

Synthesis of 6-(3-(5-chloro-2-((5-(2-oxopyrrolidin-1-yl)pyridin-3-yl)amino)pyrimidin-4-yl)piperidine-1-carbonyl)pyridin-2(1H)-one (Compound 161). To a solution of 6-(5-(5-chloro-2-((5-(2-oxopyrrolidin-1-yl)pyridin-3-yl)amino)pyrimidin-4-yl)-1,2,3,6-tetrahydropyridine-1-carbonyl)pyridin-2(1H)-one (191-2) (160 mg, 325 μmol, 1.00 eq) in MeOH (3 mL) was added PtO₂ (160 mg, 704 μmol, 2.17 eq) under N₂ atmosphere at 25° C. The suspension was degassed and purged with H₂ for 3 times. The mixture was stirred under H₂ (15 Psi) at 25° C. for 1 h. LC-MS showed the desired mass was detected. The reaction mixture was filtered and concentrated under reduced pressure. The residue was purified by prep-HPLC (column: Phenomenex luna C18 100 * 40 mm*3 um; mobile phase: [H₂O (0.2% FA)-ACN]; gradient: 1%-30% B over 8.0 min) to give the title compound (Compound 161) (12.1 mg, 24.3 μmol, 7.48% yield, 99.3% purity) as a white solid. LCMS (ES⁺): m/z 494.1 [M+H]⁺.

### Example 192 Synthesis of 1-(1-(5-chloro-2-((5-(2-oxopyrrolidin-1-yl)pyridin-3-yl)amino)pyrimidin-4-yl)piperidin-3-yl)pyridin-2(1H)-one (Compound 156)

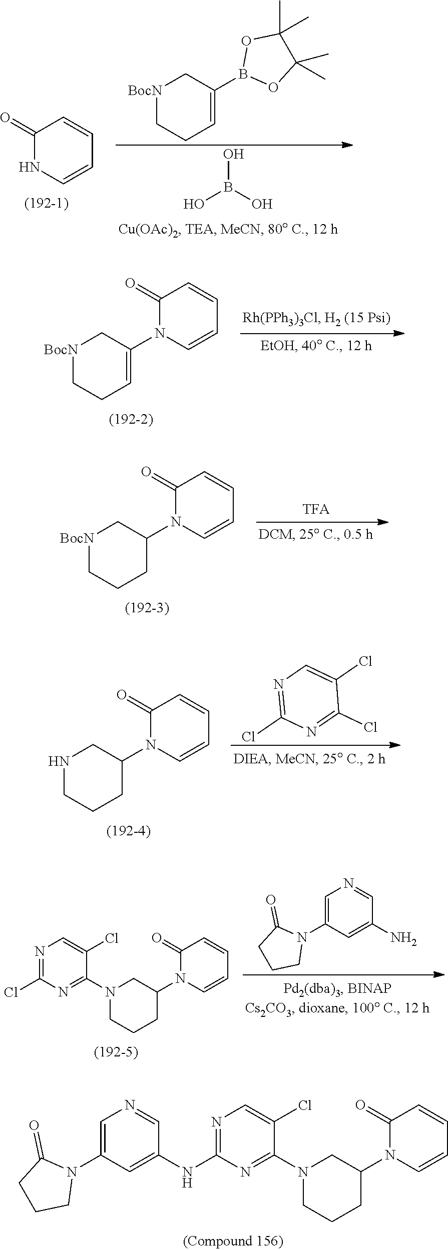

Synthesis of tert-butyl 2-oxo-5′,6′-dihydro-2H-[1,3′-bipyridine]-1′(2′H)-carboxylate (192-2). A mixture of tert-butyl 5-(4,4,5,5-tetramethyl-1,3,2-dioxaborolan-2-yl)-3,6-dihydropyridine-1(2H)-carboxylate (16.2 g, 52.5 mmol, 1.00 eq) and pyridin-2(1H)-one (192-1) (5.00 g, 52.5 mmol, 1.00 eq), boric acid (6.50 g, 105 mmol, 2.00 eq) in MeCN (500 mL) was added Cu(OAc)₂ (9.55 g, 52.5 mmol, 1.00 eq) and TEA (15.96 g, 157 mmol, 21.9 mL, 3 eq) at 25° C. The mixture was stirred at 80° C. for 12 h under air. LC-MS showed pyridin-2(1H)-one (192-1) was consumed completely and the desired mass was detected. The reaction mixture was quenched by addition H₂O 500 mL, and extracted with EtOAc (1000 mL*3). The combined organic layers were dried over Na₂SO₄, filtered and concentrated under reduced pressure. The residue was purified by column chromatography (SiO₂, Petroleum ether:Ethyl acetate=20:1 to 0:1) to give the title compound (192-2) (5.00 g, crude) as a yellow solid. LCMS (ES⁺): m/z 277.3 [M+H]⁺.

Synthesis of tert-butyl 3-(2-oxopyridin-1(2H)-yl)piperidine-1-carboxylate (192-3). A mixture of 2-oxo-5′,6′-dihydro-2H-[1,3′-bipyridine]-1′(2′H)-carboxylate (192-2) (750 mg, 2.71 mmol, 1.00 eq), chlororhodium; triphenylphosphane (502 mg, 542 μmol, 0.20 eq) in EtOH (10 mL) was degassed and purged with H₂ (15 Psi) for 3 times at 20° C., and then the mixture was stirred at 40° C. for 12 h under H₂ atmosphere. LC-MS showed the desired mass was detected. The reaction mixture was filtered and concentrated under reduced pressure to give the title compound (192-3) (750 mg, crude) as a yellow oil. LCMS (ES⁺): m/z 279.1 [M+H]⁺.

Synthesis of 1-(piperidin-3-yl)pyridin-2(1H)-one (192-4). A mixture of tert-butyl 3-(2-oxopyridin-1(2H)-yl)piperidine-1-carboxylate (192-3) (750 mg, 2.69 mmol, 1.00 eq) in DCM (1 mL) was added TFA (767 mg, 6.73 mmol, 0.50 mL, 2.50 eq) and then the mixture was stirred at 25° C. for 0.5 h. LC-MS showed the desired mass was detected. The reaction mixture was concentrated under reduced pressure to give the title compound (192-4) (480 mg, crude) as a yellow solid. LCMS (ES⁺): m/z 179.1 [M+H]⁺.

Synthesis of 1-(1-(2,5-dichloropyrimidin-4-yl)piperidin-3-yl)pyridin-2(1H)-one (192-5). To a solution of 1-(piperidin-3-yl)pyridin-2(1H)-one (192-4) (480 mg, 2.69 mmol, 1.00 eq) in MeCN (2 mL) was added DIEA (1.04 g, 8.08 mmol, 1.41 mL, 3.00 eq) and 2,4,5-trichloropyrimidine (543 mg, 2.96 mmol, 1.10 eq) at 25° C. The mixture was stirred at 25° C. for 2 h. LC-MS showed the desired mass was detected. The reaction mixture was quenched by addition H₂O 50 mL and extracted with EtOAc (50 mL*3). The combined organic layers were dried over Na₂SO₄, filtered and concentrated under reduced pressure. The residue was purified by column chromatography (SiO₂, Petroleum ether:Ethyl acetate=20:1 to 0:1) to give the title compound (192-5) (800 mg, crude) as a yellow oil. LCMS (ES⁺): m/z 325.1 [M+H]⁺.

Synthesis of 1-(1-(5-chloro-2-((5-(2-oxopyrrolidin-1-yl)pyridin-3-yl)amino)pyrimidin-4-yl)piperidin-3-yl)pyridin-2(1H)-one (Compound 156). A mixture of 1-(1-(2,5-dichloropyrimidin-4-yl)piperidin-3-yl)pyridin-2(1H)-one (192-5) (300 mg, 922 μmol, 1.00 eq), 1-(5-amino-3-pyridyl)pyrrolidin-2-one (245 mg, 1.38 mmol, 1.50 eq), Cs₂CO₃ (901 mg, 2.77 mmol, 3.00 eq), BINAP (57.4 mg, 92.2 μmol, 0.10 eq) and Pd₂(dba)₃ (84.4 mg, 92.2 μmol, 0.10 eq) in dioxane (4 mL) was degassed and purged with N₂ for 3 times at 25° C., and then the mixture was stirred at 100° C. for 12 h under N₂ atmosphere. LC-MS showed the desired mass was detected. The reaction mixture was quenched by addition H₂O 10 mL, and extracted with EtOAc (10 mL*3). The combined organic layers were dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 15%-55% B over 8.0 min] to give the title compound (Compound 156) (43.2 mg, 88.9 μmol, 9.63% yield, 95.9% purity) as an off-white solid. LCMS (ES⁺): m/z 466.0 [M+H]⁺.

### Example 193. Synthesis of 4-(3-(5-chloro-2-((5-(2-oxopyrrolidin-1-yl)pyridin-3-yl)amino)pyrimidin-4-yl)piperidine-1-carbonyl)pyridin-2(1H)-one (Compound 164)

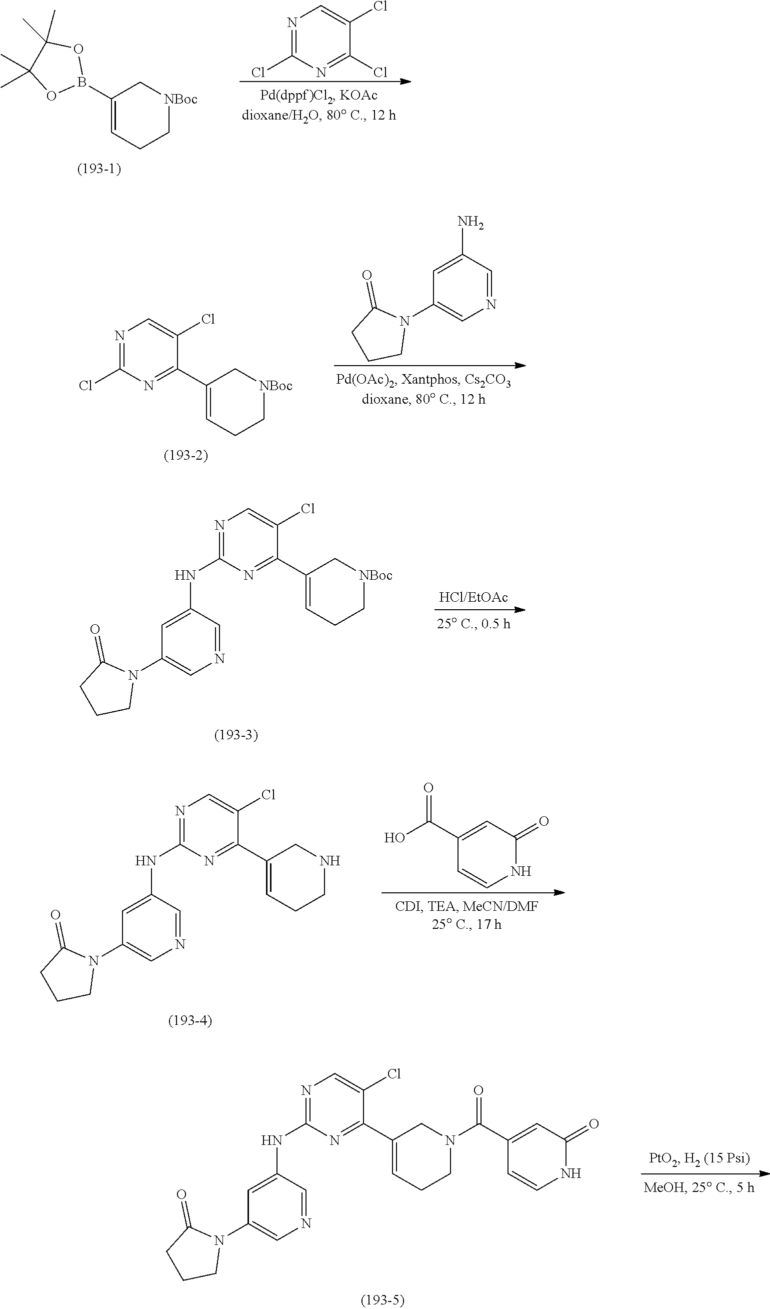

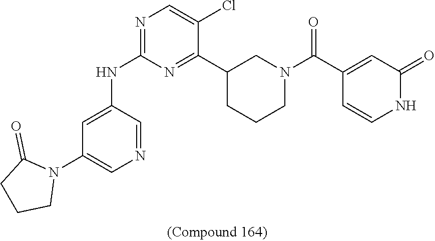 [SMILES: O=C(c1cc[nH]c(=O)c1)N1CCCC(c2nc(Nc3cncc(N4CCCC4=O)c3)ncc2Cl)C1]

Synthesis of tert-butyl 5-(2,5-dichloropyrimidin-4-yl)-3,6-dihydropyridine-1(2H)-carboxylate (193-2). A mixture of tert-butyl 5-(4,4,5,5-tetramethyl-1,3,2-dioxaborolan-2-yl)-3,6-dihydro-2H-pyridine-1-carboxylate (193-1) (5.00 g, 16.1 mmol, 1.00 eq), 2,4,5-trichloropyrimidine (2.97 g, 16.1 mmol, 1.00 eq), Pd(dppf)Cl₂ (1.18 g, 1.62 mmol, 0.100 eq), KOAc (4.76 g, 48.5 mmol, 3.00 eq) in dioxane (120 mL) and H₂O (30 mL) at 25° C. was degassed and purged with N₂ for 3 times, and then the mixture was stirred at 80° C. for 12 h under N₂ atmosphere. LC-MS showed 5-(4,4,5,5-tetramethyl-1,3,2-dioxaborolan-2-yl)-3,6-dihydro-2H-pyridine-1-carboxylate (193-1) was consumed completely and the desired mass was detected. The reaction mixture was diluted with water 150 mL and extracted with EtOAc (150 mL*3). The combined organic layers dried over Na₂SO₄, filtered and concentrated under reduced pressure. The residue was purified by column chromatography (SiO₂, Petroleum ether:Ethyl acetate=1:0 to 1:1) to give the title compound (193-2) (17.7 g, crude) as a yellow solid. LCMS (ES⁺): m/z 330.1 [M+H]⁺.

Synthesis of tert-butyl 5-(5-chloro-2-((5-(2-oxopyrrolidin-1-yl)pyridin-3-yl)amino)pyrimidin-4-yl)-3,6-dihydropyridine-1(2H)-carboxylate (193-3). A mixture of tert-butyl 5-(2,5-dichloropyrimidin-4-yl)-3,6-dihydro-2H-pyridine-1-carboxylate (193-2) (10.0 g, 30.2 mmol, 1.00 eq), 1-(5-amino-3-pyridyl)pyrrolidin-2-one (5.37 g, 30.2 mmol, 1.00 eq), Pd(OAc)₂ (679 mg, 3.03 mmol, 0.100 eq), Xantphos (1.75 g, 3.03 mmol, 0.100 eq) and Cs₂CO₃ (29.6 g, 90.8 mmol, 3.00 eq) in dioxane (100 mL) at 25° C. was degassed and purged with N₂ for 3 times, and then the mixture was stirred at 80° C. for 12 h under N₂ atmosphere. LC-MS showed tert-butyl 5-(2,5-dichloropyrimidin-4-yl)-3,6-dihydro-2H-pyridine-1-carboxylate (193-2) was consumed completely and the desired mass was detected. The reaction mixture was diluted with water 100 mL and extracted with EtOAc (100 mL*3). The combined organic layers dried over Na₂SO₄, filtered and concentrated under reduced pressure. The residue was purified by column chromatography (SiO₂, Petroleum ether:Ethyl acetate=1:0 to 1:1) to give the title compound (193-3) (12.4 g, 20.7 mmol, 68.5% yield, 78.4% purity) as a yellow solid. LCMS (ES⁺): m/z 471.2 [M+H]⁺.

Synthesis of 1-(5-((5-chloro-4-(1,2,5,6-tetrahydropyridin-3-yl)pyrimidin-2-yl)amino)pyridin-3-yl)pyrrolidin-2-one (193-4). A solution of tert-butyl 5-[5-chloro-2-[[5-(2-oxopyrrolidin-1-yl)-3-pyridyl]amino]pyrimidin-4-yl]-3,6-dihydro-2H-pyridine-1-carboxylate (193-3) (3.00 g, 6.37 mmol, 1.00 eq) in HCl/EtOAc (4 M, 40 mL, 25.1 eq) was stirred at 25° C. for 0.5 h. LC-MS showed tert-butyl 5-[5-chloro-2-[[5-(2-oxopyrrolidin-1-yl)-3-pyridyl]amino]pyrimidin-4-yl]-3,6-dihydro-2H-pyridine-1-carboxylate (193-3) was consumed completely and the desired mass was detected. The reaction mixture was concentrated under reduced pressure to give the title compound (193-4) (4.16 g, crude, HCl) as a yellow solid. LCMS (ES⁺): m/z 371.1 [M+H]⁺.

Synthesis of 4-(5-(5-chloro-2-((5-(2-oxopyrrolidin-1-yl)pyridin-3-yl)amino)pyrimidin-4-yl)-1,2,3,6-tetrahydropyridine-1-carbonyl)pyridin-2(1H)-one (193-5). 2-oxo-1,2-dihydropyridine-4-carboxylic acid (450 mg, 3.24 mmol, 1.20 eq) were suspended in a mixture of DMF (4 mL), MeCN (30 mL) and CDI (459 mg, 2.83 mmol, 1.05 eq) was added. The suspension was stirred at 25° C. for 12 h, then 1-(5-((5-chloro-4-(1,2,5,6-tetrahydropyridin-3-yl)pyrimidin-2-yl)amino)pyridin-3-yl)pyrrolidin-2-one (193-4) (1.00 g, 2.70 mmol, 1.00 eq, HCl) was added. After 3 h, TEA (136 mg, 1.35 mmol, 187 μL, 0.500 eq) was added at 25° C. The mixture was stirred at 25° C. for 2 h. LC-MS showed 1-(5-((5-chloro-4-(1,2,5,6-tetrahydropyridin-3-yl)pyrimidin-2-yl)amino)pyridin-3-yl)pyrrolidin-2-one (193-4) was consumed completely and the desired mass was detected. The reaction mixture was diluted with water 30 mL and extracted with EtOAc (30 mL*3). The combined organic layers dried over Na₂SO₄, filtered and concentrated under reduced pressure. The residue was purified by column chromatography (SiO₂, Dichloromethane:Methanol=1:0 to 1:1) to give the title compound (193-5) (391 mg, 772 μmol, 28.6% yield, 97.2% purity) as a yellow solid. LCMS (ES⁺): m/z 492.2 [M+H]⁺.

Synthesis of 4-(3-(5-chloro-2-((5-(2-oxopyrrolidin-1-yl)pyridin-3-yl)amino)pyrimidin-4-yl)piperidine-1-carbonyl)pyridin-2(1H)-one (Compound 164). To a solution of PtO₂ (152 mg, 672 μmol, 1.73 eq) in MeOH (8 mL) was added 4-(5-(5-chloro-2-((5-(2-oxopyrrolidin-1-yl)pyridin-3-yl)amino)pyrimidin-4-yl)-1,2,3,6-tetrahydropyridine-1-carbonyl)pyridin-2(1H)-one (193-5) (191 mg, 388 μmol, 1.00 eq) at 25° C. under N₂ atmosphere. The suspension was degassed and purged with H₂ for 3 times. The mixture was stirred under H₂ (15 Psi) at 25° C. for 5 h. LC-MS showed 4-(5-(5-chloro-2-((5-(2-oxopyrrolidin-1-yl)pyridin-3-yl)amino)pyrimidin-4-yl)-1,2,3,6-tetrahydropyridine-1-carbonyl)pyridin-2(1H)-one (193-5) was consumed completely and the desired mass was detected. The reaction mixture was filtered and the filtrate was concentrated under high vacuum. The residue was purified by prep-HPLC (column: 3_Phenomenex Luna C18 75*30 mm*3 um; mobile phase: [H₂O (0.1% TFA)-ACN]; gradient: 1%-30% B over 8.0 min) to give the title compound (Compound 164) (7.10 mg, 14.2 μmol, 3.67% yield, 99.0% purity) as a white solid. LCMS (ES⁺): m/z 494.1 [M+H]⁺.

### Example 194. Synthesis of N-(5-((5-chloro-4-(3-cyclopropylphenyl)pyrimidin-2-yl)amino)pyridin-3-yl)-1-(2-(4-(2-(2,6-dioxopiperidin-3-yl)-1-oxoisoindolin-5-yl)piperidin-1-yl)acetyl)piperidine-4-carboxamide (Compound 203)

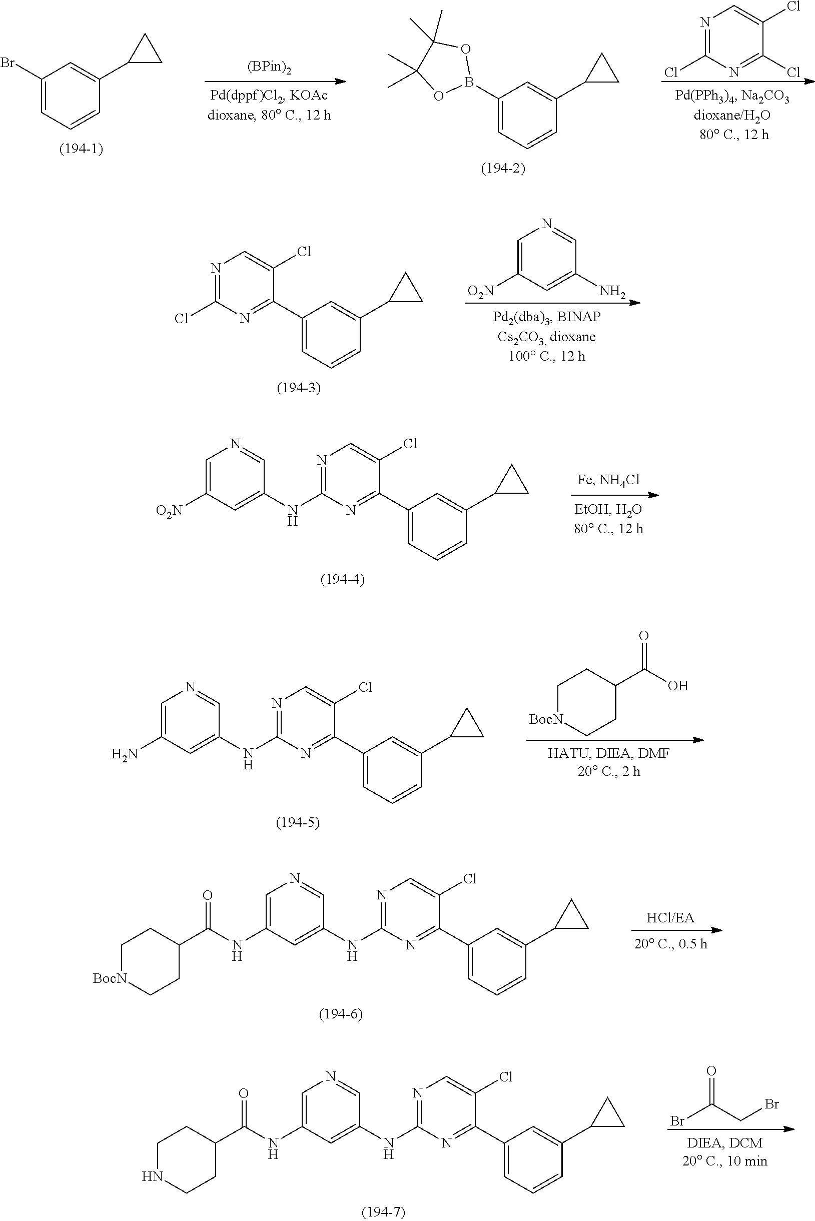

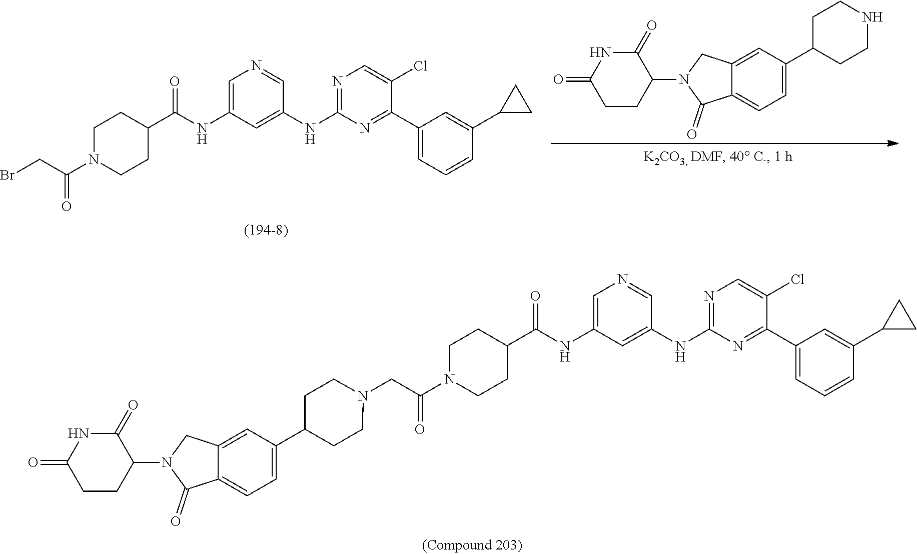

Synthesis of 2-(3-cyclopropylphenyl)-4,4,5,5-tetramethyl-1,3,2-dioxaborolane (194-2). To a solution of 1-bromo-3-cyclopropylbenzene (194-1) (7.00 g, 35.5 mmol, 1.00 eq) in dioxane (100 mL) were added 4,4,5,5-tetramethyl-2-(4,4,5,5-tetramethyl-1,3,2-dioxaborolan-2-yl)-1,3,2-dioxaborolane (10.8 g, 42.6 mmol, 1.20 eq), KOAc (6.97 g, 71.0 mmol, 2.00 eq) and Pd(dppf)Cl₂ (1.30 g, 1.78 mmol, 0.0500 eq) at 20° C. under N₂ atmosphere. The mixture was stirred at 80° C. for 12 h. TLC (SiO₂, Petroleum ether:Ethyl acetate=1:0) indicated 1-bromo-3-cyclopropylbenzene (194-1) was consumed completely and one new spot formed. Water (90 mL) was added to the mixture at 20° C. Then the reaction mixture was extracted with EtOAc (80 mL*3). The organic layers were dried over anhydrous sodium sulfate, filtered and the filtrate was concentrated under reduced pressure to give a residue. The residue was purified by MPLC (SiO₂, Petroleum ether/Ethyl acetate=1/Oto 0/1) to give the title compound (194-2) (16.3 g, 66.77 mmol, 62.7% yield) as a white oil.

Synthesis of 2,5-dichloro-4-(3-cyclopropylphenyl)pyrimidine (194-3). To a solution of 2,4,5-trichloropyrimidine (12.3 g, 66.8 mmol, 1.00 eq) in dioxane (10 mL) and H₂O (2 mL) were added 2-(3-cyclopropylphenyl)-4,4,5,5-tetramethyl-1,3,2-dioxaborolane (194-2) (16.3 g, 66.8 mmol, 1.00 eq), Pd(PPh₃)₄ (1.54 g, 1.34 mmol, 0.0200 eq), Na₂CO₃ (14.2 g, 134 mmol, 2.00 eq) at 20° C. under N₂ atmosphere. The mixture was stirred at 80° C. for 12 h. LCMS showed 2-(3-cyclopropylphenyl)-4,4,5,5-tetramethyl-1,3,2-dioxaborolane (194-2) was consumed completely and the desired mass was detected. Water (9 mL) was added to the mixture at 20° C. Then the reaction mixture was extracted with EtOAc (8 mL*3). The organic layers were dried over anhydrous sodium sulfate, filtered and the filtrate was concentrated under reduced pressure to give a residue. The mixture was purified by MPLC (SiO₂, Petroleum ether/Ethyl acetate=1/0 to 0/1) to give the title compound (194-3) (18.3 g, 40.3 mmol, 60.37% yield, 58.4% purity) as a white oil. The title compound (194-3) (10.0 g, 37.7 mmol, 1.00 eq) was purified by prep-HPLC (column: Welch Xtimate C18 250 * 100 mm #10 um; mobile phase: [H₂O (10 mM NH₄HCO₃)-ACN]; gradient: 50%-80% B over 18.0 min) to give the title compound (194-3) (4.20 g, 15.8 mmol, 42.0% yield) as a yellow oil. LCMS (ES⁺): m/z 264.9 [M+H]⁺

Synthesis of 5-chloro-4-(3-cyclopropylphenyl)-N-(5-nitropyridin-3-yl)pyrimidin-2-amine (194-4). To a stirred solution of 2,5-dichloro-4-(3-cyclopropylphenyl)pyrimidine (194-3) (4.00 g, 15.1 mmol, 1.00 eq) and 5-nitropyridin-3-amine (2.31 g, 16.6 mmol, 1.10 eq) in dioxane (60 mL) was added Cs₂CO₃ (9.83 g, 30.2 mmol, 2.00 eq), BINAP (564 mg, 905 μmol, 0.0600 eq) and Pd₂(dba)₃ (553 mg, 603 μmol, 0.0400 eq) at 20° C. The resulting mixture was stirred at 100° C. for 12 h under N₂. LCMS showed 2,5-dichloro-4-(3-cyclopropylphenyl)pyrimidine (194-3) was consumed and the desired mass was detected. The mixture was concentrated in vacuo. The mixture was purified by MPLC (SiO₂, PE/EtOAc=1/0 to 0/1) to give the title compound (194-4) (3.10 g, 8.43 mmol, 55.9% yield) as a yellow solid. LCMS (ES⁺): m/z 368.0 [M+H]⁺

Synthesis of N3-(5-chloro-4-(3-cyclopropylphenyl)pyrimidin-2-yl)pyridine-3,5-diamine (194-5). To a stirred solution of 5-chloro-4-(3-cyclopropylphenyl)-N-(5-nitropyridin-3-yl)pyrimidin-2-amine (194-4) (3.10 g, 8.43 mmol, 1.00 eq) in EtOH (40 mL) and H₂O (10 mL) was added Fe (2.35 g, 42.1 mmol, 5.00 eq) and NH₄Cl (1.35 g, 25.3 mmol, 3.00 eq) at 20° C. The resulting mixture was stirred at 80° C. for 12 h. LCMS showed 5-chloro-4-(3-cyclopropylphenyl)-N-(5-nitropyridin-3-yl)pyrimidin-2-amine (194-4) was consumed and the desired mass was detected. The mixture was filtered and the filtrate was concentrated in vacuo. The mixture was purified by MPLC (SiO₂, PE/EtOAc=1/1 to 0/1) to give the title compound (194-5) (2.50 g, 7.40 mmol, 87.80% yield) as a brown solid. LCMS (ES⁺): m/z 338.0 [M+H]⁺

Synthesis of tert-butyl 4-((5-((5-chloro-4-(3-cyclopropylphenyl)pyrimidin-2-yl)amino)pyridin-3-yl)carbamoyl)piperidine-1-carboxylate (194-6). To a stirred solution of 1-(tert-butoxycarbonyl)piperidine-4-carboxylic acid (194-5) (2.04 g, 8.88 mmol, 1.20 eq) in DMF (20 mL) was added HATU (4.22 g, 11.1 mmol, 1.50 eq), DIEA (1.91 g, 14.8 mmol, 2.58 mL, 2.00 eq) and N3-[5-chloro-4-(3-cyclopropylphenyl)pyrimidin-2-yl]pyridine-3,5-diamine (2.50 g, 7.40 mmol, 1.00 eq) at 20° C. The resulting mixture was stirred at 20° C. for 2 h. LCMS showed 1-(tert-butoxycarbonyl)piperidine-4-carboxylic acid (194-5) was consumed completely and the desired mass was detected. The mixture was diluted with H₂O (40 mL) and then extracted with EtOAc (20 mL*3). The combined organic layers were dried over Na₂SO₄, filtered and the filtrate was concentrated in vacuo. The mixture was purified by MPLC (SiO₂, PE/EtOAC=1/0 to 0/1) to give the title compound (194-6) (2.80 g, 5.10 mmol, 68.9% yield) as a yellow solid. LCMS (ES⁺): m/z 549.2 [M+H]⁺

Synthesis of N-(5-((5-chloro-4-(3-cyclopropylphenyl)pyrimidin-2-yl)amino)pyridin-3-yl)piperidine-4-carboxamide (194-7). A solution of tert-butyl 4-((5-((5-chloro-4-(3-cyclopropylphenyl)pyrimidin-2-yl)amino)pyridin-3-yl)carbamoyl)piperidine-1-carboxylate (194-6) (1.3 g, 2.37 mmol, 1.00 eq) in HCl/EtOAc (20 mL) was stirred at 20° C. for 0.5 h. LCMS showed tert-butyl 4-((5-((5-chloro-4-(3-cyclopropylphenyl)pyrimidin-2-yl)amino)pyridin-3-yl)carbamoyl)piperidine-1-carboxylate (194-6) was consumed, desired target MS was detected. The mixture was concentrated in vacuo to give the title compound (194-7) (950 mg, crude) as a white solid. LCMS (ES⁺): m/z 449.2 [M+H]⁺

Synthesis of 1-(2-bromoacetyl)-N-(5-((5-chloro-4-(3-cyclopropylphenyl)pyrimidin-2-yl)amino)pyridin-3-yl)piperidine-4-carboxamide (194-8). To a solution of N-(5-((5-chloro-4-(3-cyclopropylphenyl)pyrimidin-2-yl)amino)pyridin-3-yl)piperidine-4-carboxamide (194-7) (250 mg, 557 μmol, 1.00 eq) in DCM (2 mL) were added DIEA (216 mg, 1.67 mmol, 291 μL, 3.00 eq) at 20° C. Then 2-bromoacetyl bromide (135 mg, 668 μmol, 58.2 μL, 1.20 eq) was added to the mixture at 0° C., the mixture was stirred at 0° C. for 10 min. LCMS showed N-(5-((5-chloro-4-(3-cyclopropylphenyl)pyrimidin-2-yl)amino)pyridin-3-yl)piperidine-4-carboxamide (194-7) was consumed, desired target MS was detected. The mixture was concentrated to dryness under a stream of nitrogen to give the title compound (194-8) (317 mg, crude) as a brown oil. LCMS (ES⁺): m/z 569.2 [M+H]⁺

Synthesis of N-(5-((5-chloro-4-(3-cyclopropylphenyl)pyrimidin-2-yl)amino)pyridin-3-yl)-1-(2-(4-(2-(2,6-dioxopiperidin-3-yl)-1-oxoisoindolin-5-yl)piperidin-1-yl)acetyl)piperidine-4-carboxamide (Compound 203). To a solution of 3-(1-oxo-5-(piperidin-4-yl)isoindolin-2-yl)piperidine-2,6-dione (161 mg, 491 μmol, 1.00 eq) and 1-(2-bromoacetyl)-N-(5-((5-chloro-4-(3-cyclopropylphenyl)pyrimidin-2-yl)amino)pyridin-3-yl)piperidine-4-carboxamide (194-8) (280 mg, 491 μmol, 1.00 eq) in DMF (2 mL) was added K₂CO₃ (204 mg, 1.47 mmol, 3.00 eq) at 20° C., then the mixture was stirred at 40° C. for 1 h. LCMS showed 1-(2-bromoacetyl)-N-(5-((5-chloro-4-(3-cyclopropylphenyl)pyrimidin-2-yl)amino)pyridin-3-yl)piperidine-4-carboxamide (194-8) was consumed, desired target MS was detected. The mixture was concentrated in vacuo to give a residue. The residue was purified by prep-HPLC (column: Phenomenex luna C18 100*40 mm*5 um; mobile phase: [H₂O (0.2% FA)-ACN]; gradient: 15%-45% B over 8.0 min) to give the title compound (Compound 203) (45.0 mg, 54.1 μmol, 11.0% yield, 98.1% purity) as a yellow solid. LCMS (ES⁺): m/z 816.3 [M+H]⁺.

### Example 195. Synthesis of N-(5-((5-chloro-4-(3-cyclopropylphenyl)pyrimidin-2-yl)amino)pyridin-3-yl)-1-(2-(4-(2-(2,6-dioxopiperidin-3-yl)-1-oxoisoindolin-5-yl)piperidin-1-yl)-2-oxoethyl)piperidine-4-carboxamide (Compound 196)

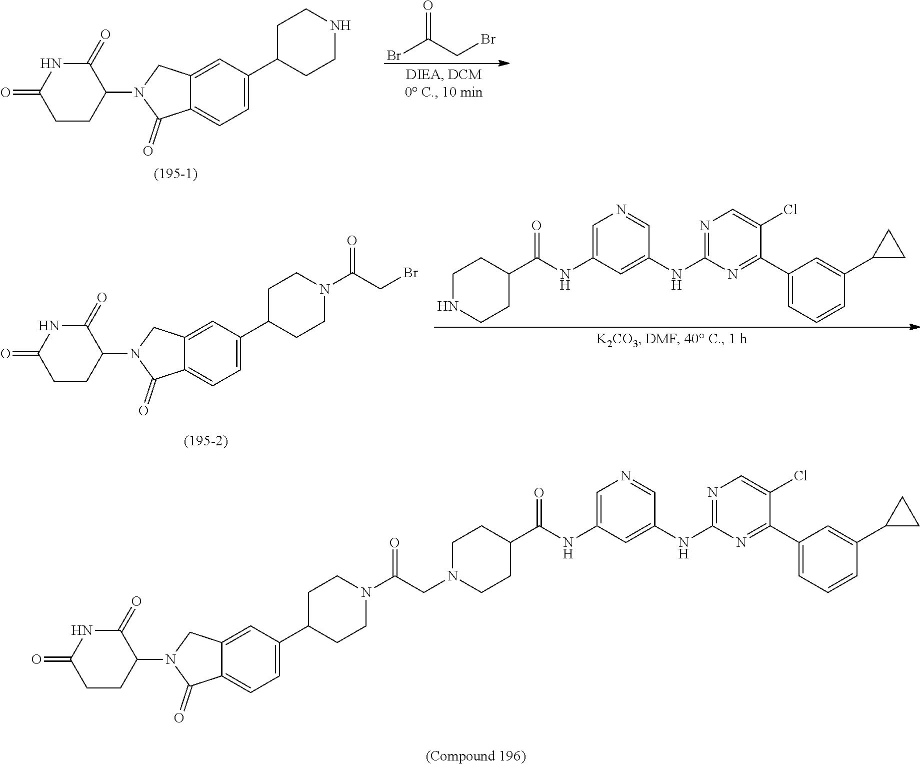

Synthesis of 3-(5-(1-(2-bromoacetyl)piperidin-4-yl)-1-oxoisoindolin-2-yl)piperidine-2,6-dione (195-2). To a solution of 3-(1-oxo-5-(piperidin-4-yl)isoindolin-2-yl)piperidine-2,6-dione (195-1) (400.0 mg, 906 μmol, 1.00 eq, TFA) in DCM (12 mL) were added DIEA (351 mg, 2.72 mmol, 474 μL, 3.00 eq) at 20° C. Then 2-bromoacetyl bromide (220 mg, 1.09 mmol, 94.7 L, 1.20 eq) was added to the mixture at 0° C., the mixture was stirred at 0° C. for 10 min. LCMS showed 3-(1-oxo-5-(piperidin-4-yl)isoindolin-2-yl)piperidine-2,6-dione (195-1) was consumed, desired target MS was detected. H₂O (8 mL) was added to the mixture and the mixture was extracted with DCM (8 mL*3), the combined organic layers were washed with saturated aqueous NaCl solution (8 mL*3), dried over Na₂SO₄, filtered and the filtrate was concentrated in vacuo to give a residue. The residue was purified by MPLC (SiO₂, Petroleum ether:Ethyl acetate=1/0 to 1/3) to give the title compound (195-2) (510 mg, crude) as a gray solid. LCMS (ES⁺): m/z 448.1 [M+H]⁺.

Synthesis of N-(5-((5-chloro-4-(3-cyclopropylphenyl)pyrimidin-2-yl)amino)pyridin-3-yl)-1-(2-(4-(2-(2,6-dioxopiperidin-3-yl)-1-oxoisoindolin-5-yl)piperidin-1-yl)-2-oxoethyl)piperidine-4-carboxamide (Compound 196) To a solution of 3-(5-(1-(2-bromoacetyl)piperidin-4-yl)-1-oxoisoindolin-2-yl)piperidine-2,6-dione (195-2) (65.0 mg, 145 μmol, 1.00 eq) and N-(5-((5-chloro-4-(3-cyclopropylphenyl)pyrimidin-2-yl)amino)pyridin-3-yl)piperidine-4-carboxamide (81.6 mg, 145 μmol, 1.00 eq, TFA) in DMF (0.2 mL) was added K₂CO₃ (60.1 mg, 435 μmol, 3.00 eq) at 20° C., then the mixture was stirred at 40° C. for 1 h. LCMS showed 3-(5-(1-(2-bromoacetyl)piperidin-4-yl)-1-oxoisoindolin-2-yl)piperidine-2,6-dione (195-2) was consumed, desired target MS was detected. The mixture was concentrated in vacuo to give a residue. The residue was purified by prep-HPLC (column: Phenomenex Luna C18 100*30 mm*3 um; mobile phase: [H₂O (0.2% FA)-ACN]; gradient: 15%-50% B over 8.0 min) to give the title compound (Compound 196) (6.30 mg, 7.63 μmol, 5.26% yield, 98.9% purity) as a white solid. LCMS (ES⁺): m/z 816.3 [M+H]⁺.

### Example 196. Synthesis of N-(5-((5-chloro-4-(3-cyclopropylphenyl)pyrimidin-2-yl)amino)pyridin-3-yl)-1-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperidine-4-carboxamide (Compound 204)

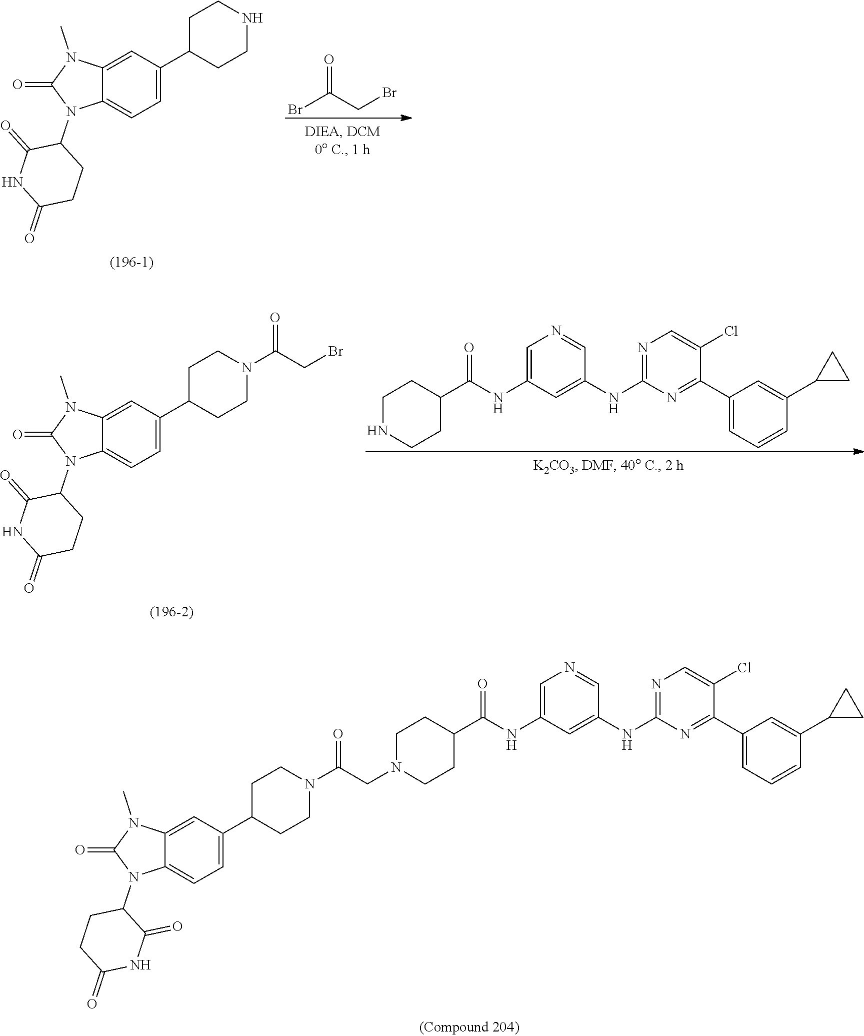 [SMILES: Cn1c(=O)n(C2CCC(=O)NC2=O)c2ccc(C3CCNCC3)cc21 ; O=C(Br)CBr ; Cn1c(=O)n(C2CCC(=O)NC2=O)c2ccc(C3CCN(C(=O)CBr)CC3)cc21 ; O=C(Nc1cncc(Nc2ncc(Cl)c(-c3cccc(C4CC4)c3)n2)c1)C1CCNCC1 ; Cn1c(=O)n(C2CCC(=O)NC2=O)c2ccc(C3CCN(C(=O)CN4CCC(C(=O)Nc5cncc(Nc6ncc(Cl)c(-c7cccc(C8CC8)c7)n6)c5)CC4)CC3)cc21]

Synthesis of 3-[5-[1-(2-bromoacetyl)-4-piperidyl]-3-methyl-2-oxo-benzimidazol-1-yl]piperidine-2,6-dione (196-2). To a solution of 3-(3-methyl-2-oxo-5-(piperidin-4-yl)-2,3-dihydro-1H-benzo[d]imidazol-1-yl)piperidine-2,6-dione (196-1) (200 mg, 438 μmol, 1.00 eq, TFA) in DCM (5 mL) were added 2-bromoacetyl bromide (106 mg, 526 μmol, 45.8 μL, 1.20 eq) and DIEA (170 mg, 1.31 mmol, 229 μL, 3.00 eq) at 0° C. The mixture was stirred at 20° C. for 1 h. LCMS showed 3-(3-methyl-2-oxo-5-(piperidin-4-yl)-2,3-dihydro-1H-benzo[d]imidazol-1-yl)piperidine-2,6-dione (196-1) was consumed, desired target MS was detected. H₂O (5 mL) was added to the solution, then the mixture was extracted with DCM (5 mL*3), the combined organic lays were dried over Na₂SO₄, then concentrated in vacuo to give a residue. The residue was purified by MPLC (SiO₂, PE:EtOAc=1:0 to 1:1) to give the title compound (196-2) (300 mg, 647 μmol, 73.9% yield) as a yellow oil. LCMS (ES⁺): m/z 463.0 [M+H]⁺.

Synthesis of N-(5-((5-chloro-4-(3-cyclopropylphenyl)pyrimidin-2-yl)amino)pyridin-3-yl)-1-(2-(4-(1-(2,6-dioxopiperidin-3-yl)-3-methyl-2-oxo-2,3-dihydro-1H-benzo[d]imidazol-5-yl)piperidin-1-yl)-2-oxoethyl)piperidine-4-carboxamide (Compound 204). To a solution of 3-(5-(1-(2-bromoacetyl)piperidin-4-yl)-3-methyl-2-oxo-2,3-dihydro-1H-benzo[d]imidazol-1-yl)piperidine-2,6-dione (196-2) (198 mg, 426 μmol, 1.50 eq) and N-[5-[[5-chloro-4-(3-cyclopropylphenyl)pyrimidin-2-yl]amino]-3-pyridyl]piperidine-4-carboxamide (160 mg, 284 μmol, 1.00 eq, TFA) in DMF (2 mL) were added K₂CO₃ (118 mg, 853 μmol, 3.00 eq) at 20° C. The mixture was stirred at 40° C. for 2 h. LCMS showed 3-(5-(1-(2-bromoacetyl)piperidin-4-yl)-3-methyl-2-oxo-2,3-dihydro-1H-benzo[d]imidazol-1-yl)piperidine-2,6-dione (196-2) was remained, desired target MS was detected. The mixture was filtered give a residue. The residue was purified by prep-HPLC (column: Waters Xbridge BEH C18 100*30 mm*10 um; mobile phase: [H₂O (10 mM NH₄HCO₃)-ACN]; gradient: 35%-65% B over 8.0 min) to give the title compound (Compound 204) (14.2 mg, 17.1 μmol, 6.01% yield, 100% purity) as a white solid. LCMS (ES⁺): m/z 831.3 [M+H]⁺.

### Example 197. Synthesis of N-(5-((5-chloro-4-(3-cyclopropylphenyl)pyrimidin-2-yl)amino)pyridin-3-yl)-1-(2-(4-(1-(2,6-dioxopiperidin-3-yl)-3-methyl-2-oxo-2,3-dihydro-1H-benzo[d]imidazol-5-yl)piperidin-1-yl)acetyl)piperidine-4-carboxamide (Compound 210)

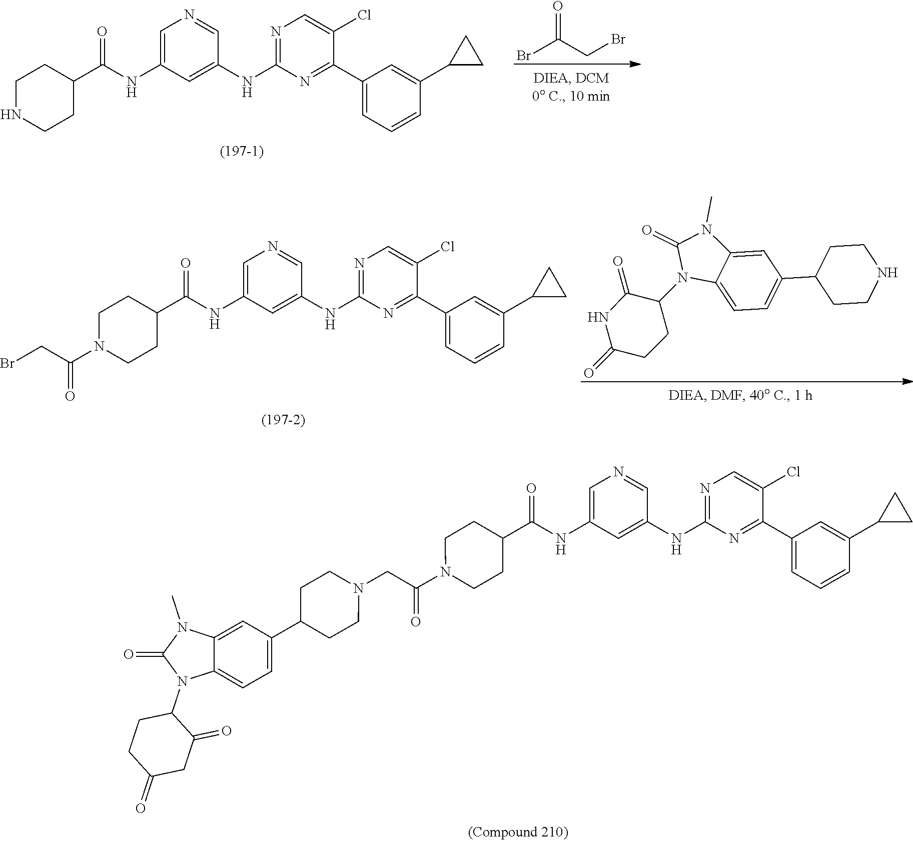

Synthesis of 1-(2-bromoacetyl)-N-(5-((5-chloro-4-(3-cyclopropylphenyl)pyrimidin-2-yl)amino)pyridin-3-yl)piperidine-4-carboxamide (197-2). To a solution of N-(5-((5-chloro-4-(3-cyclopropylphenyl)pyrimidin-2-yl)amino)pyridin-3-yl)piperidine-4-carboxamide (197-1) (460 mg, 1.02 mmol, 1.00 eq) in DCM (6 mL) were added DIEA (397 mg, 3.07 mmol, 535 μL, 3.00 eq) at 20° C. Then 2-bromoacetyl bromide (248 mg, 1.23 mmol, 107 μL, 1.20 eq) was added to the mixture at 0° C., the mixture was stirred at 0° C. for 10 min. LCMS showed N-(5-((5-chloro-4-(3-cyclopropylphenyl)pyrimidin-2-yl)amino)pyridin-3-yl)piperidine-4-carboxamide (197-1) was consumed, desired target MS was detected. The mixture was not worked up and used to next step directly. The title compound (197-2) (570 mg, crude) in DCM (6 mL) was obtained as a brown liquid. LCMS (ES⁺): m/z 569.1 [M+H]⁺

Synthesis of N-(5-((5-chloro-4-(3-cyclopropylphenyl)pyrimidin-2-yl)amino)pyridin-3-yl)-1-(2-(4-(1-(2,6-dioxopiperidin-3-yl)-3-methyl-2-oxo-2,3-dihydro-1H-benzo[d]imidazol-5-yl)piperidin-1-yl)acetyl)piperidine-4-carboxamide (Compound 210). To a solution of 1-(2-bromoacetyl)-N-(5-((5-chloro-4-(3-cyclopropylphenyl)pyrimidin-2-yl)amino)pyridin-3-yl)piperidine-4-carboxamide (197-2) (114 mg, 333 μmol, 1.00 eq) in DMF (0.7 mL) was added DIEA (86.2 mg, 667 μmol, 116 μL, 2.00 eq) at 20° C. to adjust pH to 8. Then the mixture was added to 3-(3-methyl-2-oxo-5-(piperidin-4-yl)-2,3-dihydro-1H-benzo[d]imidazol-1-yl)piperidine-2,6-dione (190 mg, 333 μmol, 1.00 eq) in DCM (2 mL), then the mixture was stirred at 40° C. for 1 h. LCMS showed 1-(2-bromoacetyl)-N-(5-((5-chloro-4-(3-cyclopropylphenyl)pyrimidin-2-yl)amino)pyridin-3-yl)piperidine-4-carboxamide (197-2) was consumed, desired target MS was detected. The mixture was concentrated in vacuo to give a residue. The residue was purified by prep-HPLC (column: Phenomenex luna C18 100 * 40 mm * 5 um; mobile phase: [H₂O (0.2% FA)-ACN]; gradient: 30%-60% B over 8.0 min) to give the title compound (Compound 210) (28.6 mg, 32.1 μmol, 9.64% yield, 98.6% purity, FA) as a white solid. LCMS (ES⁺): m/z 831.3 [M+H]⁺

### Example 198. Synthesis of 3-((4-(4-((2-(5-((5-chloro-4-(3-cyclopropylpiperidin-1-yl)pyrimidin-2-yl)amino)pyridin-3-yl)-1-oxo-2,8-diazaspiro[4.5]decan-8-yl)methyl)piperidin-1-yl)phenyl)amino)piperidine-2,6-dione (Compound 212)

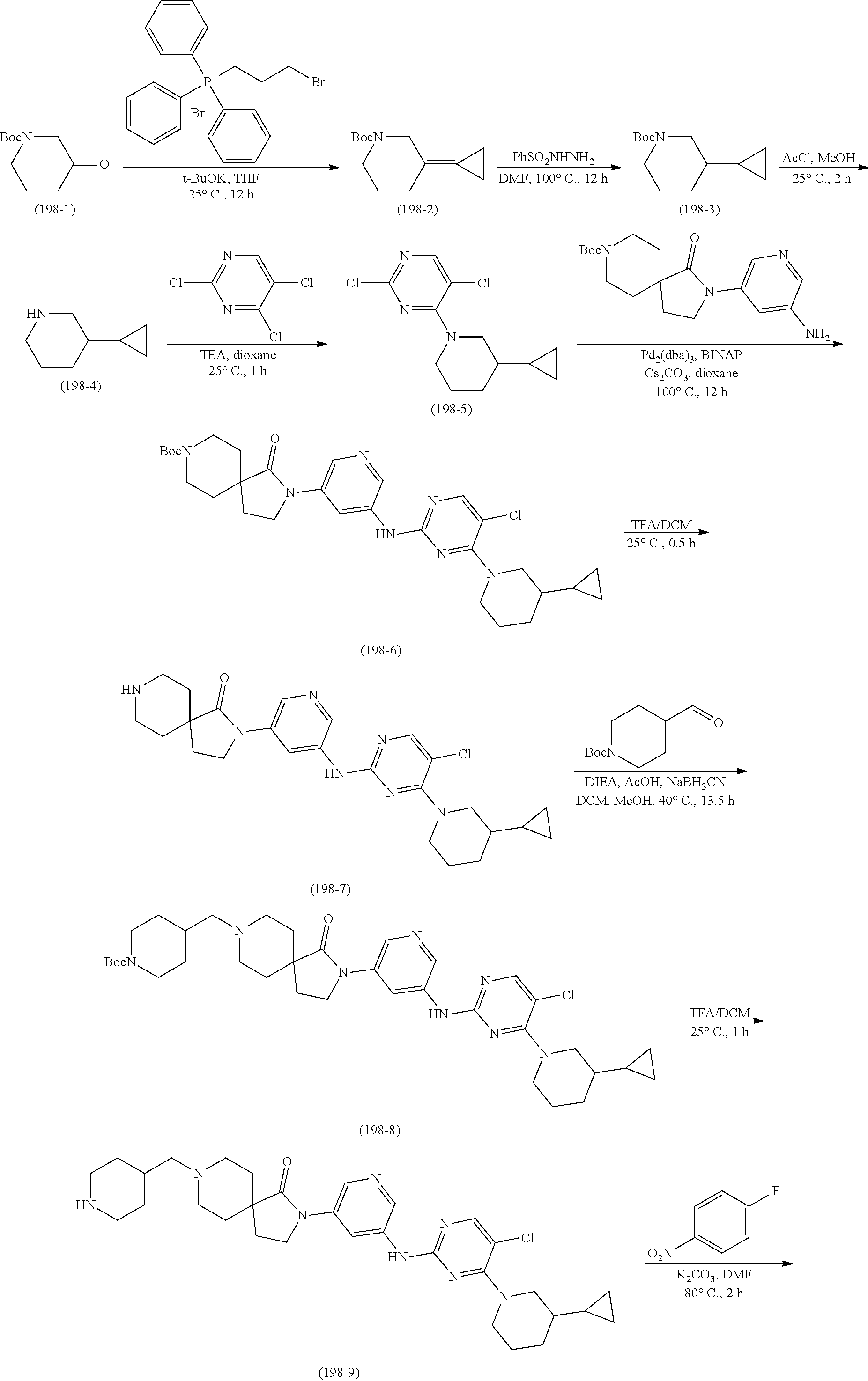

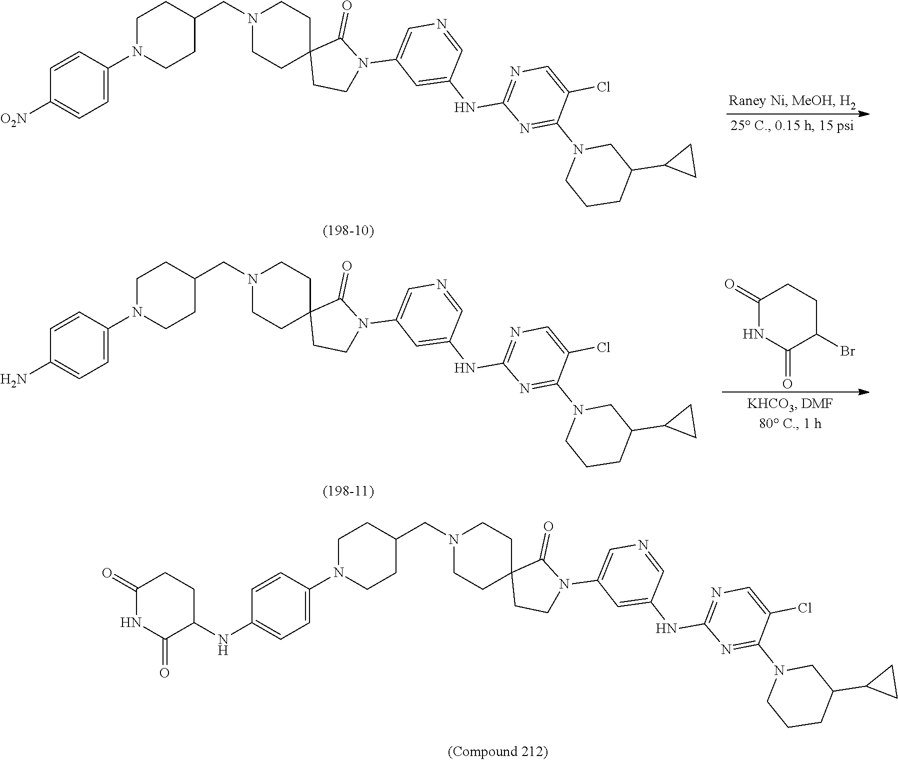 [SMILES: O=C1N(c2cncc(Nc3ncc(Cl)c(N4CCCC(C5CC5)C4)n3)c2)CCC12CCN(CC1CCN(c3ccc([N+](=O)[O-])cc3)CC1)CC2 ; Nc1ccc(N2CCC(CN3CCC4(CC3)CCN(c3cncc(Nc5ncc(Cl)c(N6CCCC(C7CC7)C6)n5)c3)C4=O)CC2)cc1 ; O=C1CCC(Br)C(=O)N1 ; O=C1CCC(Nc2ccc(N3CCC(CN4CCC5(CC4)CCN(c4cncc(Nc6ncc(Cl)c(N7CCCC(C8CC8)C7)n6)c4)C5=O)CC3)cc2)C(=O)N1]

Synthesis of tert-butyl 3-cyclopropylidenepiperidine-1-carboxylate (198-2). To a stirred solution of (3-bromopropyl)triphenylphosphonium bromide (11.2 g, 24.1 mmol, 1.20 eq) in THF (120 mL) was added t-BuOK (5.41 g, 48.2 mmol, 2.40 eq) at 0° C. and the mixture was stirred at 0° C. for 0.75 h. Then to the mixture was added tert-butyl 3-oxopiperidine-1-carboxylate (198-1) (4.00 g, 20.1 mmol, 1.00 eq) in THF (12 mL) at 0° C. and the resulting mixture was stirred at 25° C. for 12 h. TLC (SiO₂, PE/EtOAc=1/1) showed tert-butyl 3-oxopiperidine-1-carboxylate (198-1) was consumed completely and new spots were formed. The mixture was filtered and the filtrate was concentrated under reduced pressure. The mixture was triturated with hexane (60 mL) and the mixture was filtered and the filtrated was concentrated under reduced pressure to give the title compound (198-2) (3.55 g, crude) as a yellow oil.

Synthesis of tert-butyl 3-cyclopropylpiperidine-1-carboxylate (198-3). To a stirred solution of tert-butyl 3-cyclopropylidenepiperidine-1-carboxylate (198-2) (3.55 g, 15.9 mmol, 1.00 eq) in DMF (50 mL) was added benzenesulfonohydrazide (10.3 g, 59.6 mmol, 3.75 eq) at 25° C. The resulting mixture was stirred at 100° C. for 12 h. LCMS showed the desired mass was detected. The mixture was concentrated under reduced pressure and the mixture was triturated with hexane (30 mL). The mixture was filtered and then the filtrated was washed with H₂O (30 mL*3), saturated aqueous NaHCO₃ solution (30 mL*3). The organic layer was dried over Na₂SO₄, filtered and the filtrate was concentrated under reduced pressure. The mixture was purified by MPLC (SiO₂, PE/EtOAc=1/0 to 10/1) to give the title compound (198-3) (2.80 g, 12.4 mmol, 78.2% yield) as a yellow oil. LCMS (ES⁺): m/z 170.0 [M+H−56]⁺.

Synthesis of 3-cyclopropylpiperidine (198-4). To a stirred solution of tert-butyl 3-cyclopropylpiperidine-1-carboxylate (198-3) (2.80 g, 12.4 mmol, 1.00 eq) in MeOH (30 mL) was added acetyl chloride (1.63 g, 20.8 mmol, 1.48 mL, 1.67 eq) at 0° C. The resulting mixture was stirred at 25° C. for 2 h. LCMS showed the desired mass was detected. The mixture was concentrated under reduced pressure to give the title compound (198-4) (2.00 g, crude, HCl) as a yellow oil. LCMS (ES⁺): m/z 126.2 [M+H]⁺.

Synthesis of 2,5-dichloro-4-(3-cyclopropylpiperidin-1-yl)pyrimidine (198-5). To a stirred 3-cyclopropylpiperidine (198-4) (1.00 g, 6.19 mmol, 1.00 eq, HCl) in dioxane (15 mL) were added TEA (1.88 g, 18.6 mmol, 2.58 mL, 3.00 eq) and 2,4,5-trichloropyrimidine (908 mg, 4.95 mmol, 0.800 eq) at 25° C. The resulting mixture was stirred at 25° C. for 1 h. LCMS showed 3-cyclopropylpiperidine (198-4) was consumed completely and the desired mass was detected. The mixture was diluted with H₂O (20 mL) and then extracted with EtOAc (10 mL*3). The combined organic layers were dried over Na₂SO₄, filtered and the filtrate was concentrated under reduced pressure. The mixture was purified by MPLC (SiO₂, PE/EtOAC=1/0 to 5/1) to give the title compound (198-5) (1.35 g, 4.96 mmol, 40.1% yield) as a yellow oil. LCMS (ES⁺): m/z 272.0 [M+H]⁺.

Synthesis of tert-butyl 2-(5-((5-chloro-4-(3-cyclopropylpiperidin-1-yl)pyrimidin-2-yl)amino)pyridin-3-yl)-1-oxo-2,8-diazaspiro[4.5]decane-8-carboxylate (198-6). To a solution of 2,5-dichloro-4-(3-cyclopropylpiperidin-1-yl)pyrimidine (198-5) (870 mg, 3.20 mmol, 1.00 eq) and tert-butyl 2-(5-aminopyridin-3-yl)-1-oxo-2,8-diazaspiro[4.5]decane-8-carboxylate (1.11 g, 3.20 mmol, 1.00 eq) in dioxane (15 mL) were added Cs₂CO₃(2.08 g, 6.39 mmol, 2.00 eq), BINAP (119 mg, 192 μmol, 0.0600 eq) and Pd₂(dba)₃ (117 mg, 128 μmol, 0.0400 eq) at 25° C., the mixture was stirred at 80° C. for 12 h under N₂. LCMS showed 2,5-dichloro-4-(3-cyclopropylpiperidin-1-yl)pyrimidine (198-5) was consumed, desired target MS was detected. H₂O (15 mL) was added to the solution, the mixture was extracted with EtOAc (15 mL*3), then the combined organic layers were dried over Na₂SO₄, then concentrated under reduced pressure. The mixture was purified by MPLC (SiO₂, PE/EtOAc=1/0 to 1/1) to give the title compound (198-6) (1.80 g, 3.09 mmol, 96.7% yield) as a yellow solid. LCMS (ES⁺): m/z 582.3 [M+H]⁺.

Synthesis of 2-(5-((5-chloro-4-(3-cyclopropylpiperidin-1-yl)pyrimidin-2-yl)amino)pyridin-3-yl)-2,8-diazaspiro[4.5]decan-1-one (198-7). A solution of tert-butyl 2-(5-((5-chloro-4-(3-cyclopropylpiperidin-1-yl)pyrimidin-2-yl)amino)pyridin-3-yl)-1-oxo-2,8-diazaspiro[4.5]decane-8-carboxylate (198-6) (1.70 g, 2.92 mmol, 1.00 eq) in DCM (12 mL) and TFA (4 mL) was stirred at 25° C. for 0.5 h. LCMS showed tert-butyl 2-(5-((5-chloro-4-(3-cyclopropylpiperidin-1-yl)pyrimidin-2-yl)amino)pyridin-3-yl)-1-oxo-2,8-diazaspiro[4.5]decane-8-carboxylate (198-6) was consumed, desired target MS was detected. The mixture was concentrated under reduced pressure to give the title compound (198-7) (1.70 g, crude, TFA) as a yellow oil. LCMS (ES⁺): m/z 482.2 [M+H]⁺.

Synthesis of tert-butyl 4-((2-(5-((5-chloro-4-(3-cyclopropylpiperidin-1-yl)pyrimidin-2-yl)amino)pyridin-3-yl)-1-oxo-2,8-diazaspiro[4.5]decan-8-yl)methyl)piperidine-1-carboxylate (198-8). To a solution of 2-(5-((5-chloro-4-(3-cyclopropylpiperidin-1-yl)pyrimidin-2-yl)amino)pyridin-3-yl)-2,8-diazaspiro[4.5]decan-1-one (198-7) (1.70 g, 2.85 mmol, 1.00 eq, TFA) in DCM (10 mL) and MeOH (10 mL) was added DIEA (369 mg, 2.85 mmol, 497 μL, 1.00 eq) to adjust pH to 8 at 25° C. The mixture was stirred at 25° C. for 1 h. To the reaction was added AcOH (856 mg, 14.3 mmol, 816 μL, 5.00 eq) to adjust pH to 4 at 25° C. Then to the reaction mixture was added tert-butyl 4-formylpiperidine-1-carboxylate (2.43 g, 11.4 mmol, 4.00 eq) at 25° C. The mixture was stirred at 25° C. for 0.5 h. Then the reaction mixture was added NaBH₃CN (1.25 g, 20.0 mmol, 7.00 eq) at 25° C. The resulting mixture was stirred at 40° C. for 12 h. LCMS showed 2-(5-((5-chloro-4-(3-cyclopropylpiperidin-1-yl)pyrimidin-2-yl)amino)pyridin-3-yl)-2,8-diazaspiro[4.5]decan-1-one (198-7) was consumed, desired target MS was detected. The mixture was concentrated under reduced pressure. H₂O (20 mL) was added to the solution, the mixture was extracted with EtOAc (20 mL*3), then the combined organic layers were dried over Na₂SO₄, then concentrated under reduced pressure. The mixture was purified by MPLC (SiO₂, DCM/MeOH=1/0 to 1/1) to give the title compound (198-8) (1.50 g, 2.21 mmol, 77.4 yield) as a white solid. LCMS (ES⁺): m/z 679.3 [M+H]⁺.

Synthesis of 2-(5-((5-chloro-4-(3-cyclopropylpiperidin-1-yl)pyrimidin-2-yl)amino)pyridin-3-yl)-8-(piperidin-4-ylmethyl)-2,8-diazaspiro[4.5]decan-1-one (198-9). A solution of tert-butyl 4-((2-(5-((5-chloro-4-(3-cyclopropylpiperidin-1-yl)pyrimidin-2-yl)amino)pyridin-3-yl)-1-oxo-2,8-diazaspiro[4.5]decan-8-yl)methyl)piperidine-1-carboxylate (198-8) (1.50 g, 2.21 mmol, 1.00 eq) in DCM (8 mL) and TFA (4 mL) was stirred at 25° C. for 1 h. LCMS showed 4-((2-(5-((5-chloro-4-(3-cyclopropylpiperidin-1-yl)pyrimidin-2-yl)amino)pyridin-3-yl)-1-oxo-2,8-diazaspiro[4.5]decan-8-yl)methyl)piperidine-1-carboxylate (198-8) was consumed and the desired mass was detected. The mixture was concentrated under reduced pressure to give the title compound (198-9) (1.53 g, crude, TFA) as a yellow oil. LCMS (ES⁺): m/z 579.3 [M+H]⁺.

Synthesis of 2-(5-((5-chloro-4-(3-cyclopropylpiperidin-1-yl)pyrimidin-2-yl)amino)pyridin-3-yl)-8-((1-(4-nitrophenyl)piperidin-4-yl)methyl)-2,8-diazaspiro[4.5]decan-1-one (198-10). To a stirred solution of 2-(5-((5-chloro-4-(3-cyclopropylpiperidin-1-yl)pyrimidin-2-yl)amino)pyridin-3-yl)-8-(piperidin-4-ylmethyl)-2,8-diazaspiro[4.5]decan-1-one (198-9) (1.53 g, 2.21 mmol, 1.00 eq, TFA) in DMF (10 mL) were added K₂CO₃ (915 mg, 6.62 mmol, 3.00 eq) and 1-fluoro-4-nitrobenzene (311 mg, 2.21 mmol, 234 μL, 1.00 eq) at 15° C. The resulting mixture was stirred at 80° C. for 2 h. LCMS showed 2-(5-((5-chloro-4-(3-cyclopropylpiperidin-1-yl)pyrimidin-2-yl)amino)pyridin-3-yl)-8-(piperidin-4-ylmethyl)-2,8-diazaspiro[4.5]decan-1-one (198-9) was consumed completely and the desired mass was detected. The mixture was diluted with H₂O (10 mL) and then filtered. The filter cake was dried under reduced pressure to give the title compound (198-10) (1.10 g, crude) as a yellow solid. LCMS (ES⁺): m/z 700.4 [M+H]⁺.

Synthesis of 8-((1-(4-aminophenyl)piperidin-4-yl)methyl)-2-(5-((5-chloro-4-(3-cyclopropylpiperidin-1-yl)pyrimidin-2-yl)amino)pyridin-3-yl)-2,8-diazaspiro[4.5]decan-1-one (198-11). To a stirred solution of 2-(5-((5-chloro-4-(3-cyclopropylpiperidin-1-yl)pyrimidin-2-yl)amino)pyridin-3-yl)-8-((1-(4-nitrophenyl)piperidin-4-yl)methyl)-2,8-diazaspiro[4.5]decan-1-one (198-10) (200 mg, 285.60 μmol, 1.00 eq) in MeOH (3 mL) was added Raney-Ni (80.0 mg) at 25° C. under N₂. The mixture was degassed and purged with H₂ for 3 times and the resulting mixture was stirred at 25° C. for 0.15 h under H₂ (15 psi). LCMS showed 2-(5-((5-chloro-4-(3-cyclopropylpiperidin-1-yl)pyrimidin-2-yl)amino)pyridin-3-yl)-8-((1-(4-nitrophenyl)piperidin-4-yl)methyl)-2,8-diazaspiro[4.5]decan-1-one (198-10) was consumed completely and the desired mass was detected. The mixture was filtered and the filtrate was concentrated under reduced pressure to give the title compound (198-11) (120 mg, crude) as a brown oil. LCMS (ES⁺): m/z 670.4 [M+H]⁺.

Synthesis of 3-((4-(4-((2-(5-((5-chloro-4-(3-cyclopropylpiperidin-1-yl)pyrimidin-2-yl)amino)pyridin-3-yl)-1-oxo-2,8-diazaspiro[4.5]decan-8-yl)methyl)piperidin-1-yl)phenyl)amino)piperidine-2,6-dione (Compound 212). To a stirred solution of 8-((1-(4-aminophenyl)piperidin-4-yl)methyl)-2-(5-((5-chloro-4-(3-cyclopropylpiperidin-1-yl)pyrimidin-2-yl)amino)pyridin-3-yl)-2,8-diazaspiro[4.5]decan-1-one (198-11) (120 mg, 179 μmol, 1.00 eq) and 3-bromopiperidine-2,6-dione (103 mg, 537 μmol, 3.00 eq) in DMF (2 mL) were added KHCO₃ (53.8 mg, 537 μmol, 3.00 eq) at 15° C. The resulting mixture was stirred at 80° C. for 1 h. LCMS showed 8-((1-(4-aminophenyl)piperidin-4-yl)methyl)-2-(5-((5-chloro-4-(3-cyclopropylpiperidin-1-yl)pyrimidin-2-yl)amino)pyridin-3-yl)-2,8-diazaspiro[4.5]decan-1-one (198-11) was consumed and the desired mass was detected. The mixture was concentrated under reduced pressure. The mixture was purified by prep-HPLC (column: Waters Xbridge BEH C18 100*30 mm*10 um; mobile phase: [H₂O (10 mM NH₄HCO₃)-ACN]; gradient: 45%-70% B over 8.0 min) to give the title compound (Compound 212) (21.2 mg, 27.1 μmol, 15.2% yield, 100% purity) as a gray solid. LCMS (ES⁺): m/z 781.4 [M+H]⁺.

### Example 199. Synthesis of 3-((4-(1-(2-(2-(5-((4-(1-(1-acetylpiperidin-3-yl)-1H-pyrazol-4-yl)-5-chloropyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 197)

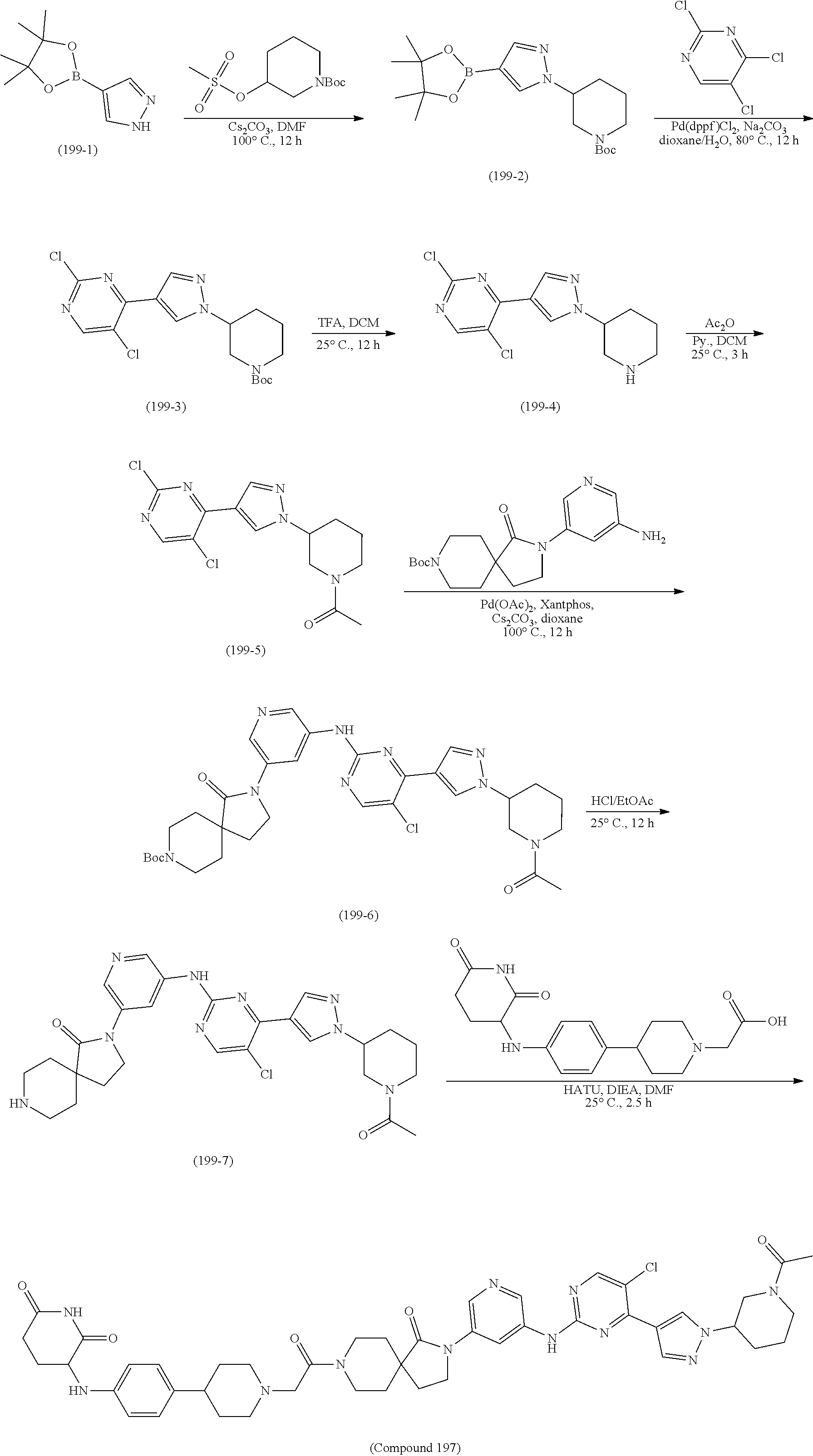

Synthesis of tert-butyl 3-(4-(4,4,5,5-tetramethyl-1,3,2-dioxaborolan-2-yl)-1H-pyrazol-1-yl)piperidine-1-carboxylate (199-2). To a solution of 4-(4,4,5,5-tetramethyl-1,3,2-dioxaborolan-2-yl)-1H-pyrazole (199-1) (8.13 g, 41.9 mmol, 1.30 eq) and tert-butyl 3-methylsulfonyloxypiperidine-1-carboxylate (9 g, 32.2 mmol, 1.00 eq) in DMF (80 mL) was added Cs₂CO₃ (26.24 g, 80.54 mmol, 2.5 eq) at 20° C. under N₂ atmosphere. The mixture was stirred at 100° C. for 12 hr under N₂ atmosphere. LC-MS showed 4-(4,4,5,5-tetramethyl-1,3,2-dioxaborolan-2-yl)-1H-pyrazole (199-1) was consumed completely and desired mass was detected. The reaction mixture was partitioned between H₂O 200 mL and EtOAc 200 mL. The organic phase was separated, the aqueous phase was washed with EtOAc (100 mL*3), dried over Na₂SO₄, filtered and concentrated under reduced pressure to give a residue. The residue was purified by prep-HPLC (column: Welch Xtimate C18 250*100 mm #10 um; mobile phase: [water (NH₄HCO₃)-ACN]; B %: 40%-75%, 20 min) to give the title compound (199-2) (2.6 g, 6.89 mmol, 21.4% yield) as a white solid. LCMS (ES⁺): m/z 378.2 [M+H]⁺.

Synthesis of tert-butyl 3-(4-(2,5-dichloropyrimidin-4-yl)-1H-pyrazol-1-yl)piperidine-1-carboxylate (199-3). To a solution of tert-butyl 3-(4-(4,4,5,5-tetramethyl-1,3,2-dioxaborolan-2-yl)-1H-pyrazol-1-yl)piperidine-1-carboxylate (199-2) (2.50 g, 6.63 mmol, 1.00 eq), 2,4,5-trichloropyrimidine (1.70 g, 9.28 mmol, 1.40 eq) and Na₂CO₃ (1.76 g, 16.57 mmol, 2.50 eq) in H₂O (10 mL) and dioxane (30 mL) was added Pd(dppf)Cl₂·CH₂Cl₂ (541.13 mg, 662.63 umol, 0.1 eq) at 20° C. under N₂ atmosphere. The mixture was stirred at 80° C. for 12 hr under N₂ atmosphere. LC-MS showed the desired mass was detected. The reaction mixture was filtered and the filtrate was concentrated under reduced pressure to remove solvent. The reaction mixture was partitioned between H₂O 60 mL and EtOAc 60 mL. The organic phase was separated, the aqueous phase was washed with EtOAc (60 mL*3), dried over Na₂SO₄, filtered and concentrated under reduced pressure to the title compound (199-3) (3 g, crude) as a brown solid, and it was used into the next step without further purification. LCMS (ES⁺): m/z 398.1 [M+H]⁺.

Synthesis of 2,5-dichloro-4-(1-(piperidin-3-yl)-1H-pyrazol-4-yl)pyrimidine (199-4). A solution of tert-butyl 3-(4-(2,5-dichloropyrimidin-4-yl)-1H-pyrazol-1-yl)piperidine-1-carboxylate (199-3) (3.00 g, 7.53 mmol, 1.00 eq) in DCM (30 mL) and TFA (6 mL) was stirred at 25° C. for 12 hr. LC-MS showed the desired mass was detected. The reaction mixture was concentrated under reduced pressure to give the title compound (199-4) (2.20 g, 7.38 mmol, 97.96% yield) as a yellow solid, and it was used into the next step without further purification. LCMS (ES⁺): m/z 298.0 [M+H]⁺.

Synthesis of 1-(3-(4-(2,5-dichloropyrimidin-4-yl)-1H-pyrazol-1-yl)piperidin-1-yl)ethan-1-one (199-5). To a solution of 2,5-dichloro-4-(1-(piperidin-3-yl)-1H-pyrazol-4-yl)pyrimidine (199-4) (2.20 g, 7.38 mmol, 1.00 eq) in DCM (20 mL) were added Py (1.17 g, 14.8 mmol, 1.19 mL, 2.00 eq) and Ac₂O (1.51 g, 14.8 mmol, 1.38 mL, 2.00 eq) at 25° C. The mixture was stirred at 25° C. for 3 hr. LC-MS showed the desired mass was detected. The reaction mixture was partitioned between H₂O 80 mL and EtOAc 80 mL. The organic phase was separated, the aqueous phase was washed with EtOAc (80 mL*3), dried over Na₂SO₄, filtered and concentrated under reduced pressure to give the title compound (199-5) (3.00 g, crude) as a white solid. LCMS (ES⁺): m/z 340.1 [M+H]⁺.

Synthesis of tert-butyl 2-(5-((4-(1-(1-acetylpiperidin-3-yl)-1H-pyrazol-4-yl)-5-chloropyrimidin-2-yl)amino)pyridin-3-yl)-1-oxo-2,8-diazaspiro[4.5]decane-8-carboxylate (199-6). A solution of 1-(3-(4-(2,5-dichloropyrimidin-4-yl)-1H-pyrazol-1-yl)piperidin-1-yl)ethan-1-one (199-5) (1.00 g, 2.94 mmol, 1.00 eq), tert-butyl 2-(5-amino-3-pyridyl)-1-oxo-2,8-diazaspiro[4.5]decane-8-carboxylate (916.44 mg, 2.65 mmol, 0.9 eq), Cs₂CO₃ (1.92 g, 5.88 mmol, 2 eq) and Xantphos (340.16 mg, 587.88 μmol, 0.2 eq) in dioxane (15 mL) was added Pd(OAc)₂ (65.99 mg, 293.94 μmol, 0.1 eq) at 20° C. under N₂ atmosphere. The mixture was stirred at 100° C. for 12 hr under N₂ atmosphere. LC-MS showed the desired mass was detected. The reaction mixture was filtered and the filterate was concentrated under reduced pressure to remove solvent. The residue was purified by prep-HPLC (column: Waters Xbridge BEH C18 250*50 mm*10 um; mobile phase: [water (NH₄HCO₃)-ACN]; B %: 35%-55%, 10 min) to give the title compound (199-6) (170 mg, 222.25 μmol, 7.56% yield, 85% purity) as a yellow solid. LCMS (ES⁺): m/z 650.2 [M+H]⁺.

Synthesis of 2-(5-((4-(1-(1-acetylpiperidin-3-yl)-1H-pyrazol-4-yl)-5-chloropyrimidin-2-yl)amino)pyridin-3-yl)-2,8-diazaspiro[4.5]decan-1-one (199-7). A solution of tert-butyl 2-(5-((4-(1-(1-acetylpiperidin-3-yl)-1H-pyrazol-4-yl)-5-chloropyrimidin-2-yl)amino)pyridin-3-yl)-1-oxo-2,8-diazaspiro[4.5]decane-8-carboxylate (199-6) (165 mg, 253.78 μmol, 1.00 eq) in HCl/EtOAc (5 mL) was stirred at 25° C. for 12 hr. LC-MS showed the desired mass was detected. The reaction mixture was concentrated under reduced pressure to give the title compound (199-7) (170 mg, crude) as a yellow solid, and it was used into the next step without further purification. LCMS (ES⁺): m/z 550.2 [M+H]⁺.

Synthesis of 3-((4-(1-(2-(2-(5-((4-(1-(1-acetylpiperidin-3-yl)-1H-pyrazol-4-yl)-5-chloropyrimidin-2-yl)amino)pyridin-3-yl)-1-oxo-2,8-diazaspiro[4.5]decan-8-yl)-2-oxoethyl)piperidin-4-yl)phenyl)amino)piperidine-2,6-dione (Compound 197). To a solution of 2-[4-[4-[[rac-(3S)-2,6-dioxo-3-piperidyl]amino]phenyl]-1-piperidyl]acetic acid (75 mg, 217.14 μmol, 1.3 eq), DIEA (86.35 mg, 668.14 μmol, 116.38 μL, 4 eq) and HATU (82.56 mg, 217.14 mol, 1.3 eq) in DMF (2 mL). After stirring 30 min at 25° C., 2-(5-((4-(1-(1-acetylpiperidin-3-yl)-1H-pyrazol-4-yl)-5-chloropyrimidin-2-yl)amino)pyridin-3-yl)-2,8-diazaspiro[4.5]decan-1-one (199-7) (91.88 mg, 167.03 μmol, 1 eq) was added to the reaction mixture at 25° C. The mixture was stirred at 25° C. for 2 hr. LC-MS showed the desired mass was detected. The reaction mixture was divided between H₂O 10 mL and EtOAc 10 mL, the organic phase was separated, the aqueous phase was washed with EtOAc 15 mL (5 mL*3), and the residue was dried on the anhydrous Na₂SO₄, filtered, and concentrated under pressure. The residue was purified by prep-HPLC (column: Phenomenex Luna C18 100*30 mm*3 um; mobile phase: [H₂O (0.2% FA)-ACN]; gradient: 10%-40% B over 8.0 min) to give the title compound (Compound 197) (33.9 mg, 36.02 μmol, 21.57% yield, 93.24% purity) as a gray solid. LCMS (ES⁺): m/z 877.1 [M+H]⁺.

### Example 200. Synthesis of N-(5-((5-chloro-4-(3-cyclopropylphenyl)pyrimidin-2-yl)amino)pyridin-3-yl)-1-(2-(4-((2-(2,6-dioxopiperidin-3-yl)-1-oxoisoindolin-5-yl)oxy)piperidin-1-yl)-2-oxoethyl)piperidine-4-carboxamide (Compound 201)

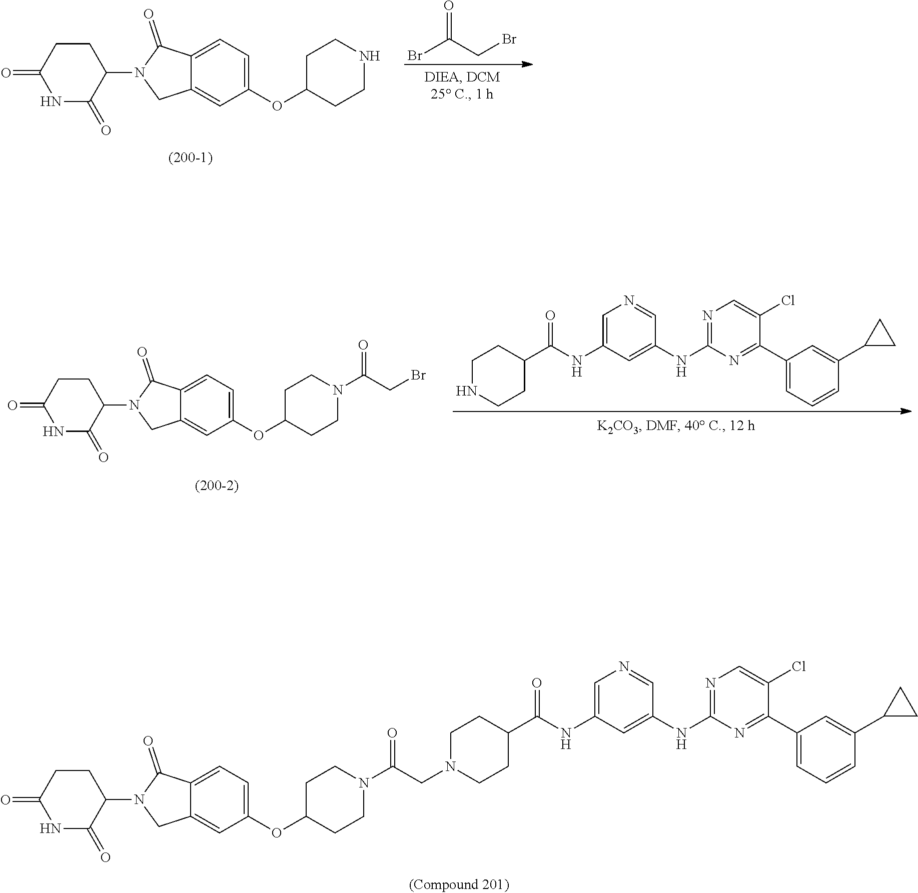 [SMILES: O=C1CCC(N2Cc3cc(OC4CCNCC4)ccc3C2=O)C(=O)N1 ; O=C(Br)CBr ; O=C1CCC(N2Cc3cc(OC4CCN(C(=O)CBr)CC4)ccc3C2=O)C(=O)N1 ; O=C(Nc1cncc(Nc2ncc(Cl)c(-c3cccc(C4CC4)c3)n2)c1)C1CCNCC1 ; O=C1CCC(N2Cc3cc(OC4CCN(C(=O)CN5CCC(C(=O)Nc6cncc(Nc7ncc(Cl)c(-c8cccc(C9CC9)c8)n7)c6)CC5)CC4)ccc3C2=O)C(=O)N1]

Synthesis of 3-(5-((1-(2-bromoacetyl)piperidin-4-yl)oxy)-1-oxoisoindolin-2-yl)piperidine-2,6-dione (200-2). To a solution of 3-(1-oxo-5-(piperidin-4-yloxy)isoindolin-2-yl)piperidine-2,6-dione (200-1) (500 mg, 1.09 mmol, 1.00 eq, TFA) in DCM (10 mL) was added DIEA (141 mg, 1.09 mmol, 190 μL, 1.00 eq) to adjust pH to 8 at 25° C. Then 2-bromoacetyl bromide (265 mg, 1.31 mmol, 114 μL, 1.20 eq) was added at 0° C., the mixture was stirred at 25° C. for 1 h. LCMS showed 3-(1-oxo-5-(piperidin-4-yloxy)isoindolin-2-yl)piperidine-2,6-dione (200-1) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in reduced pressure. The mixture was purified by prep-TLC (SiO₂, DCM/MeOH=20/1) to give the title compound (200-2) (347 mg, 747 μmol, 68.4% yield) as white solid. LCMS (ES⁺): m/z 464.1 [M+H]⁺.

Synthesis of N-(5-((5-chloro-4-(3-cyclopropylphenyl)pyrimidin-2-yl)amino)pyridin-3-yl)-1-(2-(4-((2-(2,6-dioxopiperidin-3-yl)-1-oxoisoindolin-5-yl)oxy)piperidin-1-yl)-2-oxoethyl)piperidine-4-carboxamide (Compound 201). To a solution of 3-(5-((1-(2-bromoacetyl)piperidin-4-yl)oxy)-1-oxoisoindolin-2-yl)piperidine-2,6-dione (200-2) (82.5 mg, 178 μmol, 1.00 eq) and N-(5-((5-chloro-4-(3-cyclopropylphenyl)pyrimidin-2-yl)amino)pyridin-3-yl)piperidine-4-carboxamide (100 mg, 178 μmol, 1.00 eq, TFA) in DMF (1 mL) was added K₂CO₃ (73.7 mg, 533 μmol, 3.00 eq) at 25° C., the mixture was stirred at 40° C. for 12 h. LCMS showed 3-(5-((1-(2-bromoacetyl)piperidin-4-yl)oxy)-1-oxoisoindolin-2-yl)piperidine-2,6-dione (200-2) was consumed and the desired mass was detected. To the mixture was added H₂O (10 mL) and the mixture was extracted with EtOAc (10 mL*3). The combined organic layers were washed with saturated aqueous NaCl solution (10 mL*3), dried over Na₂SO₄, filtered and the filtrate was concentrated in reduced pressure to give the crude product. The crude product was purified by neutral prep-HPLC (column: 2_Phenomenex Gemini C18 75*40 mm*3 um; mobile phase: [H₂O (10 mM NH₄HCO₃)-ACN]; gradient: 15%-45% B over 8.0 min) to give the title compound (Compound 201) (25.3 mg, 29.2 μmol, 16.4% yield, 96.0% purity) as white solid. LCMS (ES⁺): m/z 832.3 [M+H]⁺.

### Example 201. Synthesis of N-(5-((5-chloro-4-(3-cyclopropylphenyl)pyrimidin-2-yl)amino)pyridin-3-yl)-1-(2-(4-(4-((2,6-dioxopiperidin-3-yl)amino)phenyl)piperidin-1-yl)-2-oxoethyl)piperidine-4-carboxamide (Compound 202)

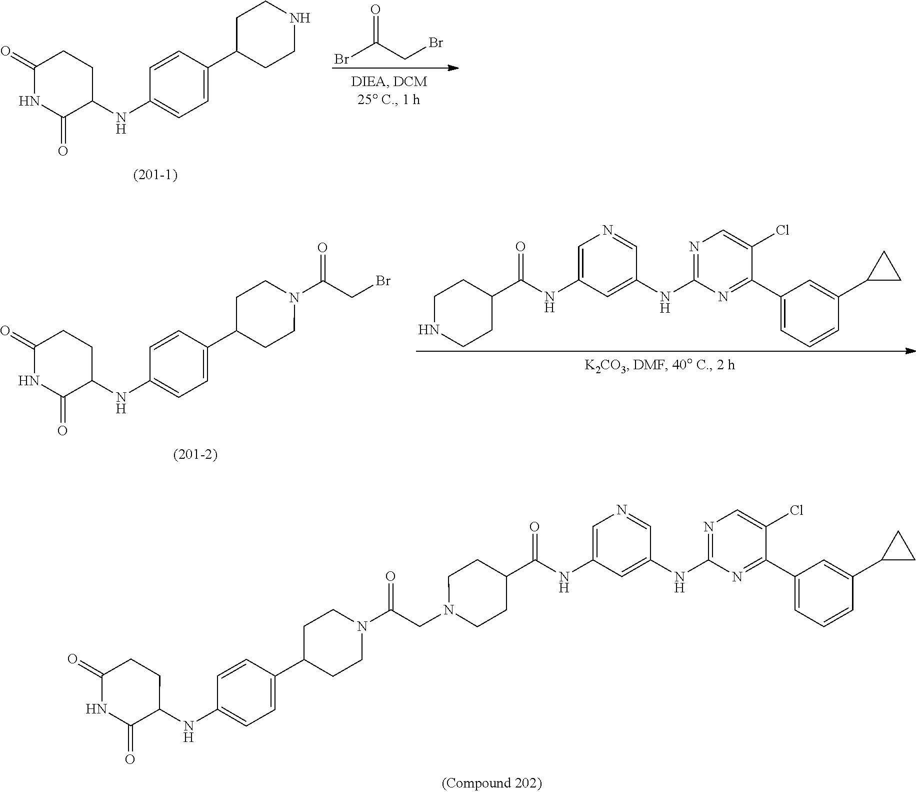 [SMILES: O=C1CCC(Nc2ccc(C3CCNCC3)cc2)C(=O)N1 ; O=C(Br)CBr ; O=C1CCC(Nc2ccc(C3CCN(C(=O)CBr)CC3)cc2)C(=O)N1 ; O=C(Nc1cncc(Nc2ncc(Cl)c(-c3cccc(C4CC4)c3)n2)c1)C1CCNCC1 ; O=C1CCC(Nc2ccc(C3CCN(C(=O)CN4CCC(C(=O)Nc5cncc(Nc6ncc(Cl)c(-c7cccc(C8CC8)c7)n6)c5)CC4)CC3)cc2)C(=O)N1]

Synthesis of 3-((4-(1-(2-bromoacetyl)piperidin-4-yl)phenyl)amino)piperidine-2,6-dione (201-2). To a solution of 3-((4-(piperidin-4-yl)phenyl)amino)piperidine-2,6-dione (201-1) (520 mg, 1.30 mmol, 1.00 eq, TFA) in DCM (6 mL) were added DIEA (502 mg, 3.89 mmol, 677 μL, 3.00 eq) and 2-bromoacetyl bromide (314 mg, 1.55 mmol, 135 μL, 1.20 eq) at 0° C. The mixture was stirred at 25° C. for 1 h. LCMS showed that 3-((4-(piperidin-4-yl)phenyl)amino)piperidine-2,6-dione (201-1) was consumed completely. The mixture was concentrated in reduced pressure. The mixture was diluted with H₂O (20 mL) and then extracted with DCM (5 mL*3). The combined organic layers were dried over Na₂SO₄, filtered and the filtrate was concentrated in reduced pressure. The residue was purified by column chromatography (SiO₂, Petroleum ether/Ethyl acetate=1/0 to 1/2) to give the title compound (201-2) (306 mg, crude) as a green solid. LCMS (ES⁺): m/z 408.3 [M+H]⁺.

Synthesis of N-(5-((5-chloro-4-(3-cyclopropylphenyl)pyrimidin-2-yl)amino)pyridin-3-yl)-1-(2-(4-(4-((2,6-dioxopiperidin-3-yl)amino)phenyl)piperidin-1-yl)-2-oxoethyl)piperidine-4-carboxamide (Compound 202). To a solution of 3-((4-(1-(2-bromoacetyl)piperidin-4-yl)phenyl)amino)piperidine-2,6-dione (201-2) (300 mg, 735 μmol, 1.50 eq) and N-(5-((5-chloro-4-(3-cyclopropylphenyl)pyrimidin-2-yl)amino)pyridin-3-yl)piperidine-4-carboxamide (276 mg, 490 μmol, 1.00 eq, TFA) in DMF (4 mL) was added K₂CO₃ (203 mg, 1.47 mmol, 3.00 eq) at 25° C. The mixture was stirred at 40° C. for 2 h. LCMS showed 3-((4-(1-(2-bromoacetyl)piperidin-4-yl)phenyl)amino)piperidine-2,6-dione (201-2) remained and desired mass was detected. H₂O (6 mL) was added to the solution, then the mixture was extracted with EtOAc (6 mL*3) and extracted with saturated NaCl solution (6 mL*3), the combined organic layers were dried over Na₂SO₄, then concentrated in reduced pressure to give the crude product. The residue was purified by FA prep-HPLC (column: Waters X bridge Prep OBD C18 150*40 mm*10 um; mobile phase: [H₂O (10 mM NH₄HCO₃)-ACN]; gradient: 40%-70% B over 8.0 min) to give the title compound (Compound 202) (21.3 mg, 25.8 μmol, 5.27% yield, 94.1% purity) as a white solid. LCMS (ES⁺): m/z 776.3 [M+H]⁺.

### Example 202. Synthesis of 3-((4-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)acetyl)piperidin-4-yl)phenyl)amino)piperidine-2,6-dione (Compound 200)

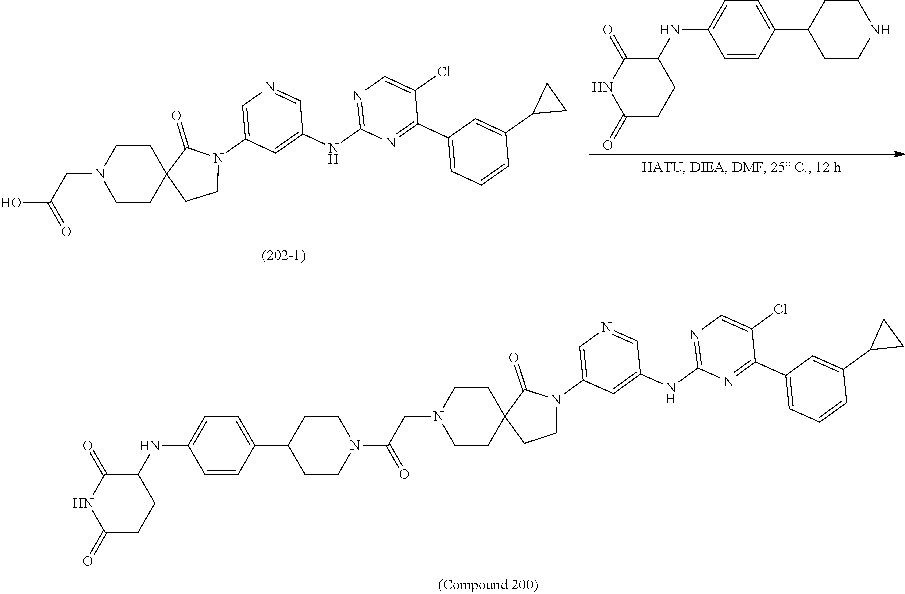 [SMILES: O=C(O)CN1CCC2(CC1)CCN(c1cncc(Nc3ncc(Cl)c(-c4cccc(C5CC5)c4)n3)c1)C2=O ; O=C1CCC(Nc2ccc(C3CCNCC3)cc2)C(=O)N1 ; O=C1CCC(Nc2ccc(C3CCN(C(=O)CN4CCC5(CC4)CCN(c4cncc(Nc6ncc(Cl)c(-c7cccc(C8CC8)c7)n6)c4)C5=O)CC3)cc2)C(=O)N1]

Synthesis of 3-((4-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)acetyl)piperidin-4-yl)phenyl)amino)piperidine-2,6-dione (Compound 200). To a solution of 3-[4-(4-piperidyl)anilino]piperidine-2,6-dione (64.7 mg, 225 μmol, 1.20 eq) and 2-[2-[5-[[5-chloro-4-(3-cyclopropylphenyl)pyrimidin-2-yl]amino]-3-pyridyl]-1-oxo-2,8-diazaspiro[4.5]decan-8-yl]acetic acid (202-1) (100 mg, 187 μmol, 1.00 eq) in DMF (3.00 mL) was added DIEA (72.7 mg, 562 μmol, 98.0 μL, 3.00 eq) and HATU (107 mg, 281 μmol, 1.50 eq) at 25° C. The mixture was stirred at 25° C. for 12 h. LC-MS showed (202-1) was consumed completely and the desired mass was detected. The reaction mixture was filtered and concentrated under reduced pressure. The residue was purified by prep-HPLC (column: Phenomenex Luna C18 75*30 mm*3 um; mobile phase: [H₂O (0.2% FA)-ACN]; gradient: 15%-55% B over 8.0 min) to give the title compound (Compound 200) (64.2 mg, 79.1 μmol, 42.1% yield, 98.9% purity) as a white solid. LCMS (ES⁺): m/z 802.3 [M+H]⁺.

### Example 203. Synthesis of 3-[5-[[1-[2-[2-[5-[[5-chloro-4-(3-cyclopropylphenyl)pyrimidin-2-yl]amino]-3-pyridyl]-1-oxo-2,8-diazaspiro[4.5]decan-8-yl]-2-oxo-ethyl]-4-piperidyl]oxy]-1-oxo-isoindolin-2-yl]piperidine-2,6-dione (Compound 205)

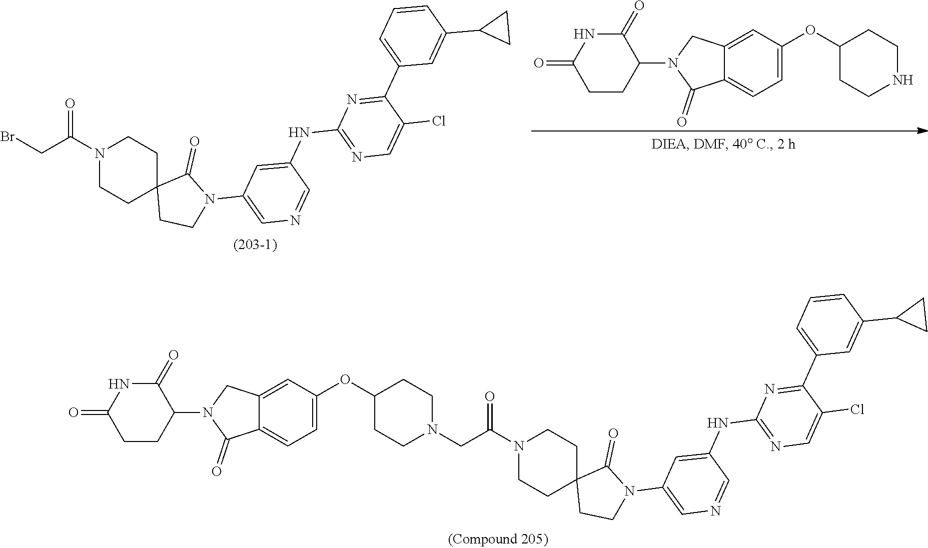

Synthesis of 3-[5-[[1-[2-[2-[5-[[5-chloro-4-(3-cyclopropylphenyl)pyrimidin-2-yl]amino]-3-pyridyl]-1-oxo-2,8-diazaspiro[4.5]decan-8-yl]-2-oxo-ethyl]-4-piperidyl]oxy]-1-oxo-isoindolin-2-yl]piperidine-2,6-dione (Compound 205). To a solution of 8-(2-bromoacetyl)-2-[5-[[5-chloro-4-(3-cyclopropylphenyl)pyrimidin-2-yl]amino]-3-pyridyl]-2,8-diazaspiro[4.5]decan-1-one (203-1) (150 mg, 251 μmol, 1.00 eq) and 3-[1-oxo-5-(4-piperidyloxy)isoindolin-2-yl]piperidine-2,6-dione (86.4 mg, 188 μmol, 0.75 eq, TFA) in DMF (3 mL) was added DIEA (97.6 mg, 755 μmol, 131 μL, 3.00 eq) at 25° C. The mixture was stirred at 40° C. for 2 h. LC-MS showed (203-1) was consumed completely and the desired mass was detected. The reaction mixture was concentrated under reduced pressure. The residue was purified by prep-HPLC (column: Phenomenex luna C18 100*40 mm*5 um; mobile phase: [H₂O (0.2% FA)-ACN]; gradient: 10%-45% B over 8.0 min) to give the title compound (Compound 205) (114.4 mg, 129 μmol, 51.4% yield, 99.4% purity, 0.44FA) as a white solid. LCMS (ES⁺): m/z 858.3 [M+H]⁺.

### Example 204. Synthesis of 3-[4-[1-[2-[2-[5-[[5-chloro-4-(3-cyclopropylphenyl)pyrimidin-2-yl]amino]-3-pyridyl]-1-oxo-2,8-diazaspiro[4.5]decan-8-yl]-2-oxo-ethyl]-4-piperidyl]anilino]piperidine-2,6-dione (Compound 206)

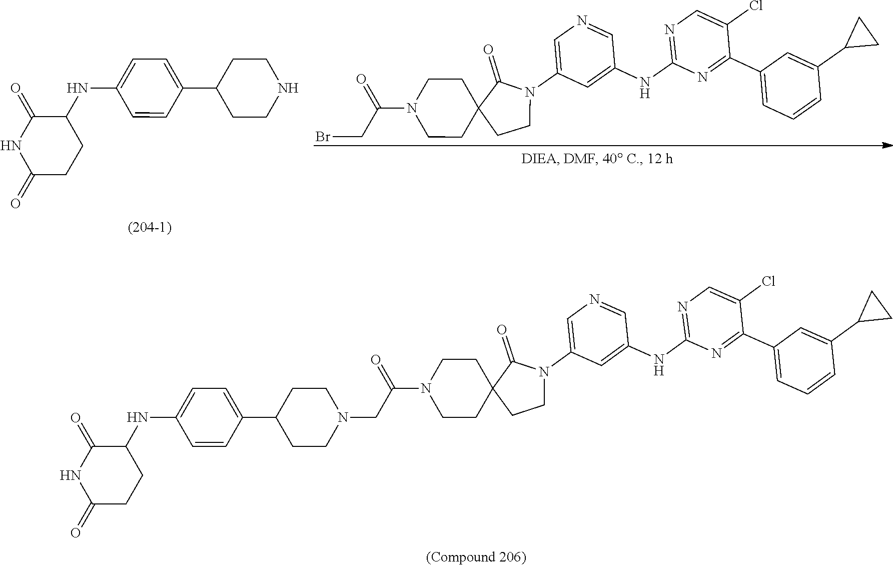 [SMILES: O=C1CCC(Nc2ccc(C3CCNCC3)cc2)C(=O)N1 ; O=C(CBr)N1CCC2(CC1)CCN(c1cncc(Nc3ncc(Cl)c(-c4cccc(C5CC5)c4)n3)c1)C2=O ; O=C1CCC(Nc2ccc(C3CCN(CC(=O)N4CCC5(CC4)CCN(c4cncc(Nc6ncc(Cl)c(-c7cccc(C8CC8)c7)n6)c4)C5=O)CC3)cc2)C(=O)N1]

Synthesis of 3-[4-[1-[2-[2-[5-[[5-chloro-4-(3-cyclopropylphenyl)pyrimidin-2-yl]amino]-3-pyridyl]-1-oxo-2,8-diazaspiro[4.5]decan-8-yl]-2-oxo-ethyl]-4-piperidyl]anilino]piperidine-2,6-dione (Compound 206). To a solution of 8-(2-bromoacetyl)-2-[5-[[5-chloro-4-(3-cyclopropylphenyl)pyrimidin-2-yl]amino]-3-pyridyl]-2,8-diazaspiro[4.5]decan-1-one (204-1) (150 mg, 251 μmol, 1.00 eq) and 3-[4-(4-piperidyl)anilino]piperidine-2,6-dione (72.3 mg, 251 μmol, 1.00 eq) in DMF (3 mL) was added DIEA (32.5 mg, 251 μmol, 43.8 μL, 1.00 eq) at 25° C. The mixture was stirred at 40° C. for 12 h. LC-MS showed (204-1) was consumed completely and the desired mass was detected. The reaction mixture was filtered and concentrated under reduced pressure. The residue was purified by prep-HPLC (column: Phenomenex Luna C18 75*30 mm*3 um; mobile phase: [H₂O (0.1% TFA)-ACN]; gradient: 20%-45% B over 8.0 min) to give the title compound (Compound 206) (145.4 mg, 177 μmol, 70.5% yield, 97.9% purity) as a white solid. LCMS (ES⁺): m/z 802.3 [M+H]⁺.

### Example 205. Synthesis of 3-[5-[1-[2-[2-[5-[[5-chloro-4-(3-cyclopropylphenyl)pyrimidin-2-yl]amino]-3-pyridyl]-1-oxo-2,8-diazaspiro[4.5]decan-8-yl]acetyl]-4-piperidyl]-3-methyl-2-oxo-benzimidazol-1-yl]piperidine-2,6-dione (Compound 208)

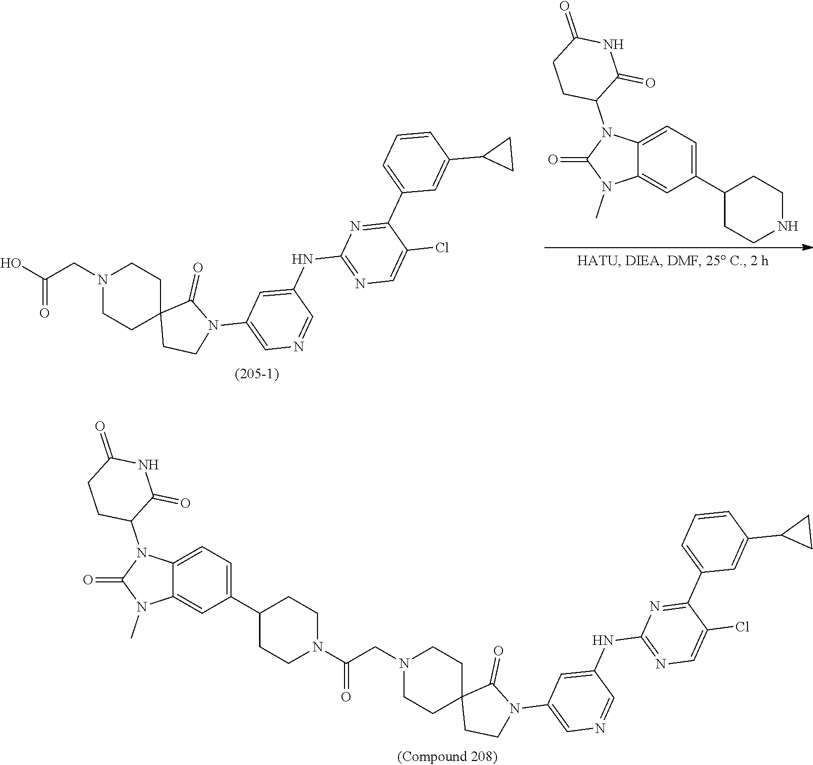

Synthesis of 3-[5-[1-[2-[2-[5-[[5-chloro-4-(3-cyclopropylphenyl)pyrimidin-2-yl]amino]-3-pyridyl]-1-oxo-2,8-diazaspiro[4.5]decan-8-yl]acetyl]-4-piperidyl]-3-methyl-2-oxo-benzimidazol-1-yl]piperidine-2,6-dione (Compound 208). To a solution of 3-[3-methyl-2-oxo-5-(4-piperidyl)benzimidazol-1-yl]piperidine-2,6-dione (77.1 mg, 225 μmol, 1.20 eq) and 2-[2-[5-[[5-chloro-4-(3-cyclopropylphenyl)pyrimidin-2-yl]amino]-3-pyridyl]-1-oxo-2,8-diazaspiro[4.5]decan-8-yl]acetic acid (205-1) (100 mg, 187 μmol, 1.00 eq) in DMF (3 mL) was added HATU (107 mg, 281 μmol, 1.50 eq) and DIPEA (72.7 mg, 562 μmol, 98.0 μL, 3.00 eq) at 25° C. The mixture was stirred at 25° C. for 2 h. LC-MS showed (205-1) was consumed completely and the desired mass was detected. The reaction mixture was concentrated under reduced pressure. The residue was purified by prep-HPLC (column: Phenomenex Luna C18 75*30 mm*3 um; mobile phase: [H₂O (0.2% FA)-ACN]; gradient: 20%-55% B over 8.0 min) to give the title compound (Compound 208) (32.4 mg, 37.2 μmol, 19.8% yield, 98.4% purity) was obtained as a white solid. LCMS (ES⁺): m/z 857.3 [M+H]⁺.

### Example 206. Synthesis of 3-(5-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)-2-oxoethyl)piperidin-4-yl)-3-methyl-2-oxo-2,3-dihydro-1H-benzo[d]imidazol-1-yl)piperidine-2,6-dione (Compound 209)

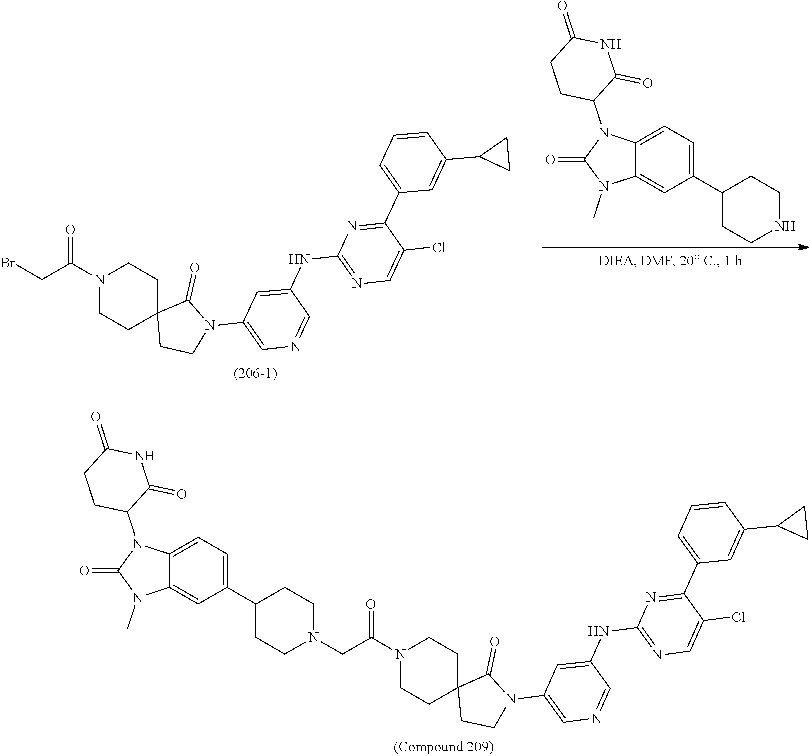

Synthesis of 3-(5-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)-2-oxoethyl)piperidin-4-yl)-3-methyl-2-oxo-2,3-dihydro-1H-benzo[d]imidazol-1-yl)piperidine-2,6-dione (Compound 209). To a solution of 8-(2-bromoacetyl)-2-(5-((5-chloro-4-(3-cyclopropylphenyl)pyrimidin-2-yl)amino)pyridin-3-yl)-2,8-diazaspiro[4.5]decan-1-one (206-1) (200 mg, 335 μmol, 1.00 eq) in DMF (3 mL) was added DIEA (130 mg, 1.01 mmol, 175 μL, 3.00 eq) and 3-(3-methyl-2-oxo-5-(piperidin-4-yl)-2,3-dihydro-1H-benzo[d]imidazol-1-yl)piperidine-2,6-dione (114 mg, 335 μmol, 1.00 eq) at 20° C. The mixture was stirred at 20° C. for 1 h. LC-MS showed 8-(2-bromoacetyl)-2-(5-((5-chloro-4-(3-cyclopropylphenyl)pyrimidin-2-yl)amino)pyridin-3-yl)-2,8-diazaspiro[4.5]decan-1-one (206-1) was consumed completely and the desired mass was detected. The reaction mixture was filtered and the filtrate concentrated under high vacuum. The residue was purified by prep-HPLC (Phenomenex luna C18 100*40 mm*5 um; mobile phase: [H₂O (0.2% FA)-ACN]; gradient: 10%-45% B over 8.0 min) to give the title compound (Compound 209) (167 mg, 188 μmol, 56.2% yield, 96.3% purity) as a white solid. LCMS (ES⁺): m/z 857.4 [M+H]⁺.

### Example 207. Synthesis of 3-(5-((1-(2-(2-(5-((5-chloro-4-(3-cyclopropylphenyl)pyrimidin-2-yl)amino)pyridin-3-yl)-1-oxo-2,8-diazaspiro[4.5]decan-8-yl)acetyl)piperidin-4-yl)oxy)-1-oxoisoindolin-2-yl)piperidine-2,6-dione (Compound 199)

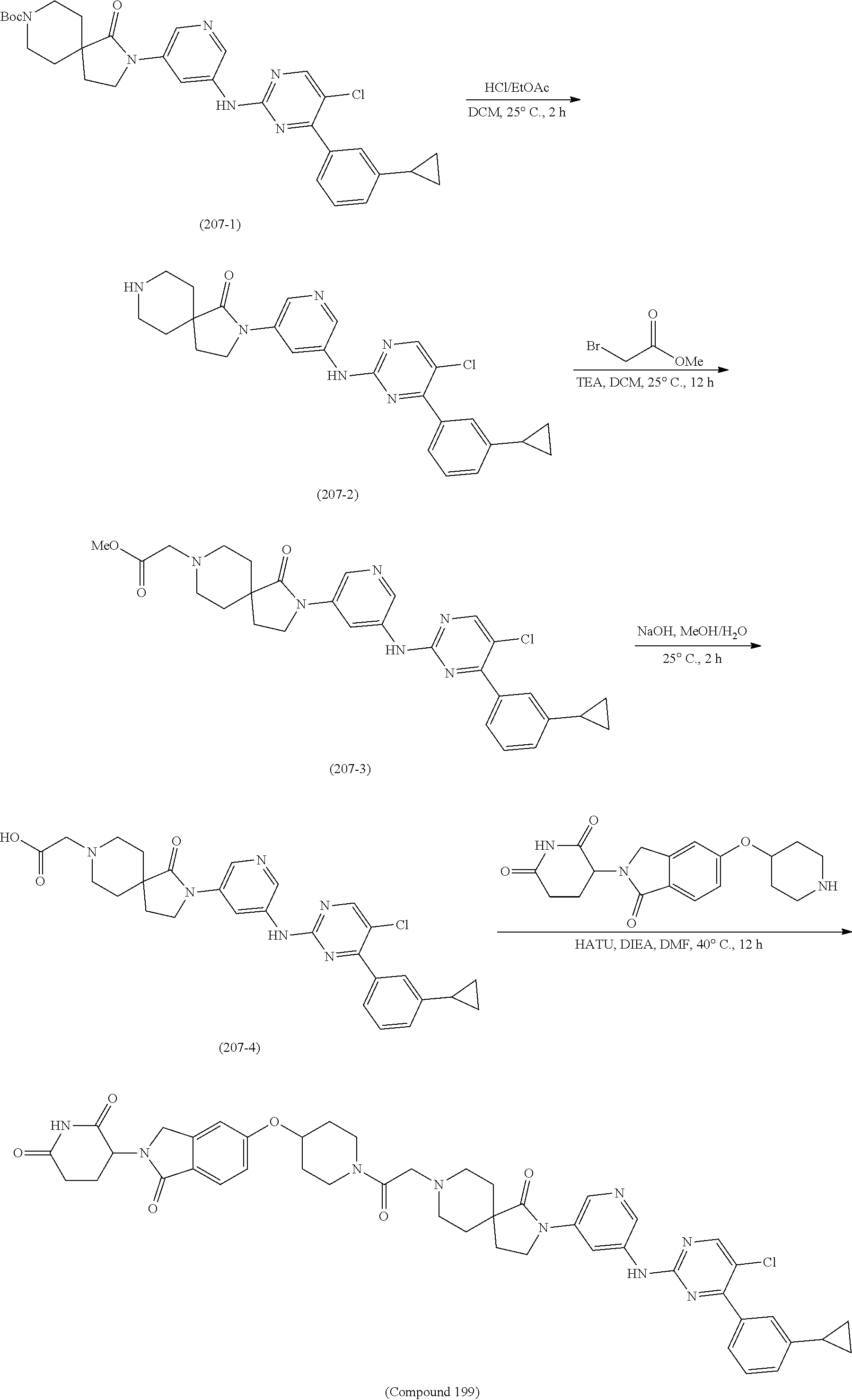

Synthesis of 2-[5-[[5-chloro-4-(3-cyclopropylphenyl)pyrimidin-2-yl]amino]-3-pyridyl]-2,8-diazaspiro[4.5]decan-1-one (207-2). To a solution of tert-butyl 2-[5-[[5-chloro-4-(3-cyclopropylphenyl)pyrimidin-2-yl]amino]-3-pyridyl]-1-oxo-2,8-diazaspiro[4.5]decane-8-carboxylate (207-1) (3.00 g, 5.22 mmol, 1.00 eq) in DCM (2 mL) was added HCl/EtOAc (4.00 M, 3.91 mL, 3.00 eq) at 25° C. The mixture was stirred at 25° C. for 2 h. LC-MS showed tert-butyl 2-(5-((5-chloro-4-(3-cyclopropylphenyl)pyrimidin-2-yl)amino)pyridin-3-yl)-1-oxo-2,8-diazaspiro[4.5]decane-8-carboxylate (207-1) was consumed completely and desired mass was detected. The reaction concentrated under reduced pressure to give the title compound (207-2) (3.00 g, crude) as a white solid. LCMS (ES⁺): m/z 475.2 [M+H]⁺.

Synthesis of methyl 2-[2-[5-[[5-chloro-4-(3-cyclopropylphenyl)pyrimidin-2-yl]amino]-3-pyridyl]-1-oxo-2,8-diazaspiro[4.5]decan-8-yl]acetate (207-3). To a solution of 2-[5-[[5-chloro-4-(3-cyclopropylphenyl)pyrimidin-2-yl]amino]-3-pyridyl]-2,8-diazaspiro[4.5]decan-1-one (207-2) (1.20 g, 2.53 mmol, 1.00 eq) in DCM (10 mL) was added TEA (766 mg, 7.58 mmol, 1.05 mL, 3.00 eq) and methyl 2-bromoacetate (579 mg, 3.79 mmol, 358 μL, 1.50 eq) at 25° C. The mixture was stirred at 25° C. for 12 h. LC-MS showed 2-[5-[[5-chloro-4-(3-cyclopropylphenyl)pyrimidin-2-yl]amino]-3-pyridyl]-2,8-diazaspiro[4.5]decan-1-one (207-2) was consumed completely and the desired mass was detected. The reaction diluted with H₂O 50 mL and combined organic layers were extracted with EtOAc (100 mL*3), dried over Na₂SO₄, filtered and concentrated under reduced pressure. The residue was purified by column chromatography (SiO₂, Petroleum ether:Ethyl acetate=1:0 to 5:1) to give the title compound (207-3) (900 mg, 1.22 mmol, 48.1% yield, 73.8% purity) as a brown solid. LCMS (ES⁺): m/z 547.2 [M+H]⁺.

Synthesis of 2-[2-[5-[[5-chloro-4-(3-cyclopropylphenyl)pyrimidin-2-yl]amino]-3-pyridyl]-1-oxo-2,8-diazaspiro[4.5]decan-8-yl]acetic acid (207-4). To a solution of methyl 2-[2-[5-[[5-chloro-4-(3-cyclopropylphenyl)pyrimidin-2-yl]amino]-3-pyridyl]-1-oxo-2,8-diazaspiro[4.5]decan-8-yl]acetate (207-3) (800 mg, 1.46 mmol, 2.00 eq) in MeOH (15 mL) and H₂O (3 mL) was added NaOH (58.4 mg, 1.46 mmol, 2.00 eq) at 25° C. The mixture was stirred at 25° C. for 2 h. LC-MS showed methyl 2-[2-[5-[[5-chloro-4-(3-cyclopropylphenyl)pyrimidin-2-yl]amino]-3-pyridyl]-1-oxo-2,8-diazaspiro[4.5]decan-8-yl]acetate (207-3) was consumed completely and desired mass was detected. The reaction diluted with H₂O 50 mL and combined organic layers were extracted with EtOAc (100 mL*3), dried over Na₂SO₄, filtered and concentrated under reduced pressure to give the title compound (207-4) (600 mg, crude) as a white solid. LCMS (ES⁺): m/z 533.3 [M+H]⁺.

Synthesis of 3-(5-((1-(2-(2-(5-((5-chloro-4-(3-cyclopropylphenyl)pyrimidin-2-yl)amino)pyridin-3-yl)-1-oxo-2,8-diazaspiro[4.5]decan-8-yl)acetyl)piperidin-4-yl)oxy)-1-oxoisoindolin-2-yl)piperidine-2,6-dione (Compound 199). To a solution of 2-[2-[5-[[5-chloro-4-(3-cyclopropylphenyl)pyrimidin-2-yl]amino]-3-pyridyl]-1-oxo-2,8-diazaspiro[4.5]decan-8-yl]acetic acid (207-4) (100 mg, 187 μmol, 1.00 eq) in DMF (2 mL) was added DIEA (72.7 mg, 562 μmol, 98.0 μL, 3.00 eq), 3-[1-oxo-5-(4-piperidyloxy)isoindolin-2-yl]piperidine-2,6-dione (64.4 mg, 187 μmol, 1.00 eq) and HATU (107 mg, 281 μmol, 1.50 eq) at 25° C. The mixture was stirred at 40° C. for 12 h. LC-MS showed 2-[2-[5-[[5-chloro-4-(3-cyclopropylphenyl)pyrimidin-2-yl]amino]-3-pyridyl]-1-oxo-2,8-diazaspiro[4.5]decan-8-yl]acetic acid (207-4) was consumed completely and the desired mass was detected. The reaction concentrated under reduced pressure. The residue was purified by prep-HPLC (column: Phenomenex Luna C18 75*30 mm*3 um; mobile phase: [H₂O (0.2% FA)-ACN]; gradient: 15%-50% B over 8.0 min) to give the title compound (Compound 199) (99.1 mg, 112 μmol, 60.2% yield, 99.4% purity, 0.300 FA) as a white solid. LCMS (ES⁺): m/z 858.3 [M+H]⁺.

### Example 208. 3-(5-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)acetyl)piperidin-4-yl)-1-oxoisoindolin-2-yl)piperidine-2,6-dione (Compound 198)

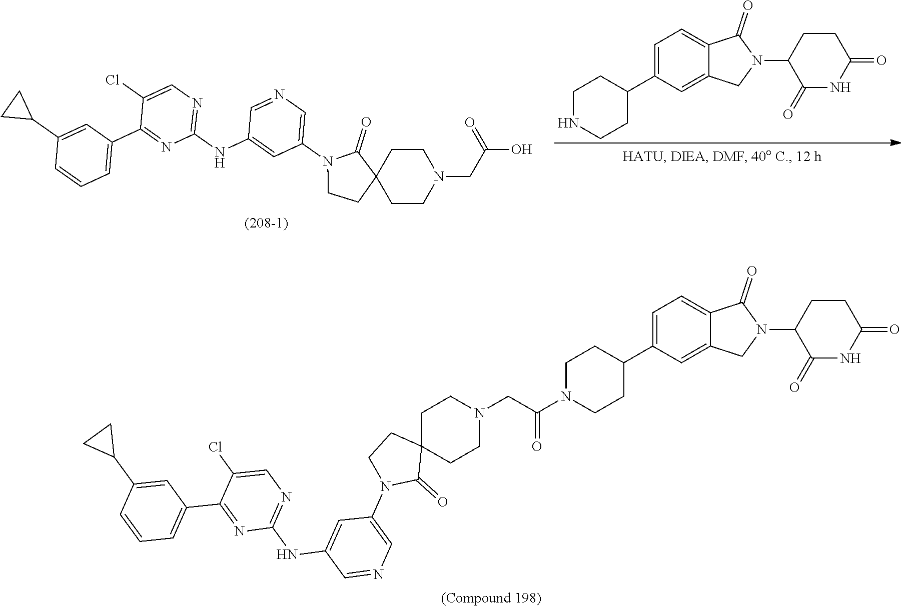 [SMILES: O=C(O)CN1CCC2(CC1)CCN(c1cncc(Nc3ncc(Cl)c(-c4cccc(C5CC5)c4)n3)c1)C2=O ; O=C1CCC(N2Cc3cc(C4CCNCC4)ccc3C2=O)C(=O)N1 ; O=C1CCC(N2Cc3cc(C4CCN(C(=O)CN5CCC6(CC5)CCN(c5cncc(Nc7ncc(Cl)c(-c8cccc(C9CC9)c8)n7)c5)C6=O)CC4)ccc3C2=O)C(=O)N1]

Synthesis of 3-(5-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)acetyl)piperidin-4-yl)-1-oxoisoindolin-2-yl)piperidine-2,6-dione (Compound 198). To a solution of 2-[2-[5-[[5-chloro-4-(3-cyclopropylphenyl)pyrimidin-2-yl]amino]-3-pyridyl]-1-oxo-2,8-diazaspiro[4.5]decan-8-yl]acetic acid (208-1) (100 mg, 187 μmol, 1.00 eq) in DMF (2 mL) was added HATU (71.3 mg, 187 μmol, 1.00 eq), 3-[1-oxo-5-(4-piperidyl)isoindolin-2-yl]piperidine-2,6-dione (61.4 mg, 187 μmol, 1.00 eq) and DIEA (24.2 mg, 187 μmol, 32.6 μL, 1.00 eq) at 25° C. The mixture was stirred at 40° C. for 12 h. LC-MS showed 2-[2-[5-[[5-chloro-4-(3-cyclopropylphenyl)pyrimidin-2-yl]amino]-3-pyridyl]-1-oxo-2,8-diazaspiro[4.5]decan-8-yl]acetic acid (208-1) was consumed completely and desired mass was detected. The reaction concentrated under reduced pressure. The residue was purified by prep-HPLC (column: Phenomenex luna C18 100*40 mm*5 um; mobile phase: [H₂O (0.2% FA)-ACN]; gradient: 15%-55% B over 8.0 min) to give the title compound (Compound 198) (62.1 mg, 72.8 μmol, 38.8% yield, 99.7% purity, 0.17 FA) as a white solid. LCMS (ES⁺): m/z 842.3 [M+H]⁺.

### Example 209. Synthesis of 3-(5-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)-2-oxoethyl)piperidin-4-yl)-1-oxoisoindolin-2-yl)piperidine-2,6-dione (Compound 207)

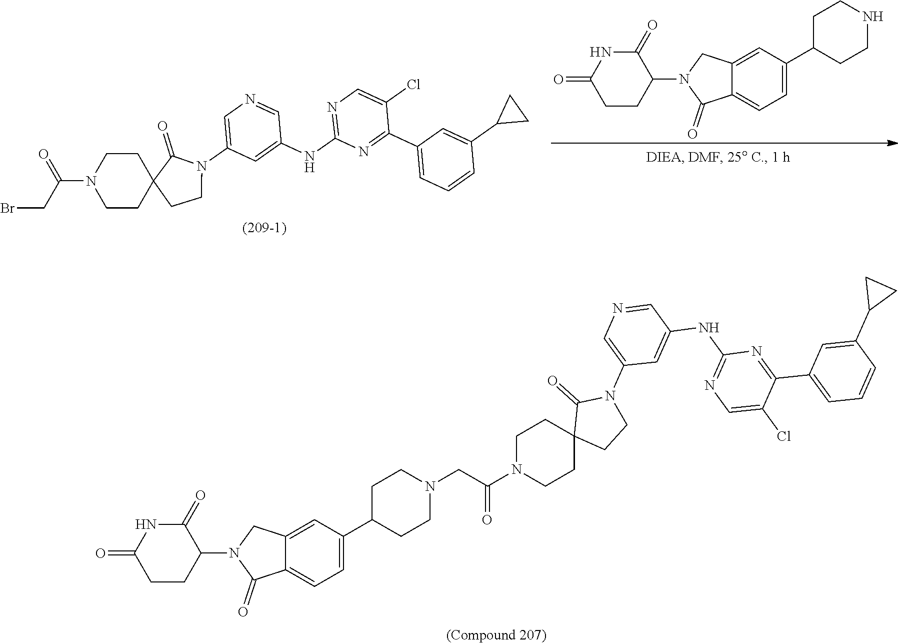 [SMILES: O=C(CBr)N1CCC2(CC1)CCN(c1cncc(Nc3ncc(Cl)c(-c4cccc(C5CC5)c4)n3)c1)C2=O ; O=C1CCC(N2Cc3cc(C4CCNCC4)ccc3C2=O)C(=O)N1 ; O=C1CCC(N2Cc3cc(C4CCN(CC(=O)N5CCC6(CC5)CCN(c5cncc(Nc7ncc(Cl)c(-c8cccc(C9CC9)c8)n7)c5)C6=O)CC4)ccc3C2=O)C(=O)N1]

Synthesis of 3-(5-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)-2-oxoethyl)piperidin-4-yl)-1-oxoisoindolin-2-yl)piperidine-2,6-dione (Compound 207). A mixture of 3-[1-oxo-5-(4-piperidyl)isoindolin-2-yl]piperidine-2,6-dione (90.6 mg, 276 μmol, 1.10 eq), 8-(2-bromoacetyl)-2-[5-[[5-chloro-4-(3-cyclopropylphenyl)pyrimidin-2-yl]amino]-3-pyridyl]-2,8-diazaspiro[4.5]decan-1-one (209-1) (150 mg, 251 μmol, 1.00 eq) in DMF (1 mL) was added DIEA (65.1 mg, 503 μmol, 87.69 μL, 2.00 eq) at 25° C. The mixture was stirred at 25° C. for 1 h. LC-MS showed 8-(2-bromoacetyl)-2-[5-[[5-chloro-4-(3-cyclopropylphenyl)pyrimidin-2-yl]amino]-3-pyridyl]-2,8-diazaspiro[4.5]decan-1-one (209-1) was consumed completely and the desired mass was detected. The reaction mixture was quenched by addition H₂O 10 mL, and then diluted with EtOAc 10 mL and extracted with EtOAc (10 mL*3). The combined organic layers were dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 20%-50% B over 8.0 min] to give the title compound (Compound 207) (43.5 mg, 50.8 μmol, 20.18% yield, 100.00% purity, 0.3FA) as a yellow solid. LCMS (ES⁺): m/z 842.3 [M+H]⁺.

### Example 210. Synthesis of 3-((4-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)ethyl)piperidin-4-yl)phenyl)amino)piperidine-2,6-dione (Compound 211)

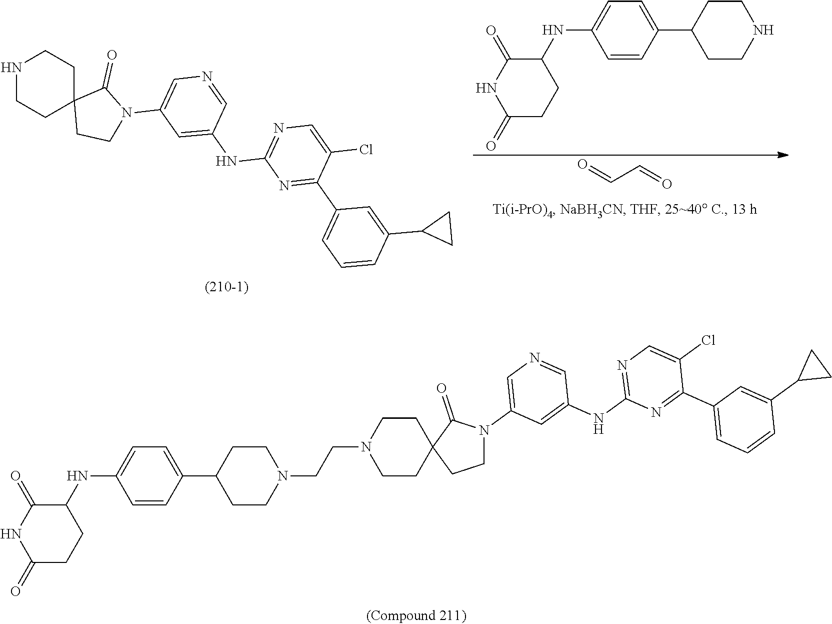

Synthesis of 3-((4-(1-(2-(2-(5-((5-chloro-4-(3-cyclopropylphenyl)pyrimidin-2-yl)amino)pyridin-3-yl)-1-oxo-2,8-diazaspiro[4.5]decan-8-yl)ethyl)piperidin-4-yl)phenyl)amino)piperidine-2,6-dione (Compound 211). To a solution of 2-[5-[[5-chloro-4-(3-cyclopropylphenyl)pyrimidin-2-yl]amino]-3-pyridyl]-2,8-diazaspiro[4.5]decan-1-one (210-1) (100 mg, 210 μmol, 1.00 eq) and 3-[4-(4-piperidyl)anilino]piperidine-2,6-dione (60.5 mg, 210 μmol, 1.00 eq) in THF (5 mL) was added DIEA (81.6 mg, 631 μmol, 110 μL, 3.00 eq) at 25° C., the mixture was stirred at 25° C. for 0.5 h. Then Ti(i-PrO)₄ (299 mg, 1.05 mmol, 310 μL, 5.00 eq) and oxaldehyde (12.2 mg, 210 μmol, 10.9 μL, 1.00 eq) was added at 25° C., and the mixture was stirred at 25° C. for 0.5 h. Then NaBH₃CN (66.1 mg, 1.05 mmol, 5.00 eq) was added at 25° C., the mixture was stirred at 40° C. for 12 h. LC-MS showed the desired mass was detected. The reaction mixture was diluted with H₂O 10 mL, and extracted with EtOAc (20 mL*3). The combined organic layers were dried over Na₂SO₄, filtered and concentrated under reduced pressure. The residue was purified by prep-HPLC (column: Phenomenex luna C18 100*40 mm*5 um; mobile phase: [H₂O (0.2% FA)-ACN]; gradient: 5%-40% B over 8.0 min) to give the title compound (Compound 211) (2.80 mg, 3.34 μmol, 1.59% yield, 97.4% purity, 0.6FA) as a white solid. LCMS (ES⁺): m/z 788.3 [M+H]⁺.

TABLE 3
Spectrometric data for compounds of Table 1

| Cmpd No | MS calc | MS found | ¹H NMR Data (400 MHz, DMSO-d₆) |
|---|---|---|---|
| 1 | 510 | 511 | δ 8.76-8.69 (m, 2H), 8.63 (d, J = 2.2 Hz, 2H), 8.34 (s, 1H), 8.16 (s, |
| | | | 1H), 7.89 (dd, J = 12.9, 8.1 Hz, 2H), 7.76 (d, J = 7.3 Hz, 2H), 7.69- |
| | | | 7.63 (m, 1H), 7.55-7.50 (m, 2H), 7.46-7.39 (m, 1H), 3.72-3.64 |
| | | | (m, 2H), 3.10-2.98 (m, 2H), 2.77-2.65 (m, 2H), 1.95-1.86 (m, |
| | | | 2H), 1.78-1.67 (m, 2H), 1.45 (br d, J = 12.6 Hz, 2H) |
| 2 | 509 | 510 | δ 10.24 (s, 1H), 8.73 (s, 1H), 8.68 (s, 1H), 8.62 (d, J = 2.0 Hz, 1H), |
| | | | 8.55 (d, J = 2.3 Hz, 1H), 8.19 (s, 1H), 8.05 (s, 1H), 7.92 (d, J = 7.5 |
| | | | Hz, 2H), 7.77 (s, 2H), 7.71-7.64 (m, 1H), 3.74 (t, J = 7.0 Hz, 2H), |
| | | | 2.41 (t, J = 8.0 Hz, 2H), 1.92 (quin, J = 7.5 Hz, 2H) |
| 3 | 455 | 456 | δ 10.23 (s, 1H), 8.72 (s, 1H), 8.65-8.62 (m, 2H), 8.58 (d, J = 1.9 Hz, |
| | | | 1H), 8.12 (s, 1H), 7.85 (t, J = 8.1 Hz, 2H), 7.64 (d, J = 7.9 Hz, 3H), |
| | | | 7.32 (d, J = 7.9 Hz, 2H), 3.73 (t, J = 6.9 Hz, 2H), 2.42 (t, J = 8.1 Hz, |
| | | | 2H), 2.37 (s, 3H), 1.91 (quin, J = 7.3 Hz, 2H) |
| 4 | 475 | 476 | δ 10.73 (s, 1H), 8.89 (br d, J = 7.3 Hz, 2H), 8.84-8.78 (m, 2H), 8.15 |
| | | | (s, 1H), 7.93-7.86 (m, 2H), 7.79 (d, J = 8.5 Hz, 2H), 7.72-7.63 (m, |
| | | | 1H), 7.57 (d, J = 8.5 Hz, 2H), 3.79 (br t, J = 7.0 Hz, 2H), 2.48 (br s, |
| | | | 2H), 1.97 (quin, J = 7.4 Hz, 2H) |
| 5 | 471 | 472 | δ 10.23 (s, 1H), 8.72 (s, 1H), 8.64 (br s, 2H), 8.58 (s, 1H), 8.10 (s, |
| | | | 1H), 7.85-7.80 (m, 2H), 7.70 (d, J = 8.5 Hz, 2H), 7.65-7.58 (m, |
| | | | 1H), 7.07 (d, J = 8.5 Hz, 2H), 3.82 (s, 3H), 3.74 (br t, J = 6.9 Hz, 2H), |
| | | | 2.43 (t, J = 8.0 Hz, 2H), 1.92 (quin, J = 7.3 Hz, 2H) |
| 6 | 475 | 476 | δ 10.22 (s, 1H), 8.71 (s, 1H), 8.66-8.58 (m, 2H), 8.56-8.48 (m, |
| | | | 1H), 7.98-7.88 (m, 2H), 7.77 (d, J = 8.3 Hz, 1H), 7.54-7.42 (m, |
| | | | 5H), 3.76 (t, J = 7.0 Hz, 2H), 2.46 (t, J = 8.0 Hz, 2H), 2.02 (quin, J = |
| | | | 7.5 Hz, 2H) |
| 7 | 455 | 456 | δ 10.16 (s, 1H), 8.68-8.63 (m, 2H), 8.60-8.56 (m, 1H), 8.54 (d, J = |
| | | | 2.1 Hz, 1H), 8.15 (s, 1H), 7.85 (dd, J = 7.9, 1.7 Hz, 1H), 7.74 (d, J = |
| | | | 1.7 Hz, 1H), 7.52-7.46 (m, 3H), 7.42 (d, J = 7.4 Hz, 3H), 3.76 (t, J = |
| | | | 7.0 Hz, 2H), 2.46 (t, J = 8.0 Hz, 2H), 2.33 (s, 3H), 2.06 (br s, 2H) |
| 8 | 471 | 472 | δ 10.14 (s, 1H), 10.17-10.11 (m, 1H), 8.67-8.64 (m, 2H), 8.60 (t, |
| | | | J = 2.0 Hz, 1H), 8.55 (d, J = 2.0 Hz, 1H), 8.00 (m, 1H), 7.90 (d, J = 2.3 |
| | | | Hz, 1H), 7.55 (d, J = 7.3 Hz, 2H), 7.45 (t, J = 7.5 Hz, 2H), 7.40-7.35 |
| | | | (m, 1H), 7.30 (d, J = 8.8 Hz, 1H), 3.88 (s, 3H), 3.74 (t, J = 7.0 Hz, |
| | | | 2H), 3.32 (s, 3H), 2.44 (t, J = 8.1 Hz, 2H), 1.98 (m, 2H) |
| 9 | 442 | 443 | δ 10.26 (s, 1H), 8.76-8.69 (m, 2H), 8.68-8.57 (m, 4H), 8.28 (d, J = |
| | | | 8.0 Hz, 1H), 8.07 (d, J = 8.0 Hz, 1H), 7.99-7.90 (m, 2H), 7.69 (t, J = |
| | | | 7.8 Hz, 1H), 7.42 (dd, J = 7.1, 5.1 Hz, 1H), 3.76 (t, J = 7.0 Hz, 2H), |
| | | | 2.43 (t, J = 8.1 Hz, 2H), 1.90 (br t, J = 7.4 Hz, 2H) |
| 10 | 455 | 456 | δ 10.22 (s, 1H), 8.73 (s, 1H), 8.64 (d, J = 2.1 Hz, 1H), 8.55-8.50 (m, |
| | | | 2H), 7.75-7.69 (m, 4H), 7.49-7.45 (m, 3H), 7.40-7.35 (m, 1H), |
| | | | 3.75 (t, J = 7.0 Hz, 2H), 2.45 (t, J = 8.0 Hz, 2H), 2.25 (s, 3H), 2.00- |
| | | | 1.92 (m, 2H) |
| 11 | 455 | 456 | δ 8.77 (t, J = 2.3 Hz, 1H), 8.51 (d, J = 2.1 Hz, 1H), 8.46-8.41 (m, |
| | | | 2H), 7.82 (s, 2H), 7.61 (s, 1H), 7.57-7.53 (m, 2 H), 7.47 (s, 1H), |
| | | | 7.38 (t, J = 7.5 Hz, 2H), 7.32-7.27 (m, 1H), 3.68 (t, J = 7.0 Hz, 2H), |
| | | | 2.50-2.41 (m, 5H), 1.93-1.90 (m, 2H) |
| 12 | 405 | 406 | δ 10.17 (s, 1H), 8.68-8.62 (m, 3H), 8.55 (d, J = 2.1 Hz, 1H), 7.63 (d, |
| | | | J = 7.9 Hz, 1H), 7.56 (s, 1H), 7.42 (t, J = 7.7 Hz, 1H), 7.24 (d, J = 7.8 |
| | | | Hz, 1H), 3.84 (t, J = 7.0 Hz, 2H), 3.31 (s, 5H), 2.54 (br s, 1H), 2.15- |
| | | | 2.00 (m, 4H), 1.04-0.98 (m, 2H), 0.76-0.71 (m, 2H) |
| 13 | 405 | 406 | δ 10.48 (s, 1H), 8.84 (d, J = 1.9 Hz, 1H), 8.77 (t, J = 1.9 Hz, 1H), |
| | | | 8.74 (d, J = 2.0 Hz, 1H), 8.70 (s, 1H), 7.81 (d, J = 8.3 Hz, 2H), 7.25 |
| | | | (d, J = 8.3 Hz, 2H), 3.86 (t, J = 7.0 Hz, 2H), 2.57 (t, J = 8.1 Hz, 2H), |
| | | | 2.12 (quin, J = 7.5 Hz, 2H), 2.07-1.99 (m, 1H), 1.09-1.01 (m, 2H), |
| | | | 0.81-0.75 (m, 2H) |
| 14 | 475 | 476 | δ 10.20 (s, 1H), 8.71 (s, 1H), 8.66 (d, J = 2.1 Hz, 1H), 8.61 (t, J = 2.1 |
| | | | Hz, 1H), 8.53 (d, J = 2.3 Hz, 1H), 7.98-7.92 (m, 2H), 7.71-7.59 (m, |
| | | | 3H), 7.52-7.42 (m, 3H), 3.78 (t, J = 6.9 Hz, 2H), 2.45 (t, J = 8.1 Hz, |
| | | | 2H), 2.01 (quin, J = 7.5 Hz, 2H) |
| 15 | 455 | 456 | δ 10.22 (s, 1H), 8.72 (s, 1H), 8.67 (s, 1H), 8.62 (s, 1H), 8.56 (s, 1H), |
| | | | 7.93 (br d, J = 7.6 Hz, 1H), 7.84 (s, 1H), 7.65 (t, J = 7.7 Hz, 1H), 7.56 |
| | | | (br d, J = 7.7 Hz, 1H), 7.39-7.26 (m, 4H), 3.79 (br t, J = 6.9 Hz, 2H), |
| | | | 2.47 (br t, J = 8.0 Hz, 2H), 2.31 (s, 3H), 2.02 (quin, J = 7.5 Hz, 2H) |
| 16 | 442 | 443 | δ 10.31 (s, 1H), 9.01 (s, 1H), 8.79-8.71 (m, 2H), 8.70-8.54 (m, |
| | | | 3H), 8.28-8.19 (m, 2H), 7.96 (dd, J = 7.9, 1.4 Hz, 2H), 7.72 (t, J = |
| | | | 7.7 Hz, 1H), 7.59 (dd, J = 7.7, 5.0 Hz, 1H), 3.76 (br t, J = 6.8 Hz, |
| | | | 2H), 2.43 (t, J = 8.1 Hz, 2H), 1.97-1.87 (m, 2H) |
| 17 | 443 | 444 | δ 10.27 (s, 1H), 9.36 (d, J = 1.2 Hz, 1H), 8.79-8.77 (m, 1H), 8.74 (s, |
| | | | 1H), 8.69 (t, J = 2.5 Hz, 2H), 8.63 (s, 2H), 8.57 (d, J = 2.3 Hz, 1H), |
| | | | 8.34 (d, J = 7.9 Hz, 1H), 8.02 (d, J = 7.7 Hz, 1H), 7.74 (t, J = 7.8 Hz, |
| | | | 1H), 3.77 (t, J = 7.0 Hz, 2H), 2.42 (t, J = 8.0 Hz, 2H), 1.99-1.88 (m, 2H) |
| 18 | 471 | 472 | δ 10.23 (s, 1 H), 8.72 (s, 1H), 8.66-8.58 (m, 3H), 7.78-7.70 (m, |
| | | | 3H), 7.54-7.47 (m, 2H), 7.46-7.38 (m, 3H), 3.92 (s, 3 H), 3.74 (br |
| | | | t, J = 6.8 Hz, 2H), 2.42 (br t, J = 8.0 Hz, 2H), 1.86-1.97 (m, 2H), |
| | | | 1.24 (br s, 1H) |
| 19 | 447 | 448 | δ 10.19 (s, 1H), 8.70-8.67 (m, 2H), 8.65 (d, J = 2.0 Hz, 1H), 8.54 (d, |
| | | | J = 2.1 Hz, 1H), 7.72-7.67 (m, 2H), 7.50-7.41 (m, 2H), 3.85 (t, J = |
| | | | 7.0 Hz, 2H), 2.67-2.58 (m, 1H), 2.55 (br s, 2H), 2.11 (quin, J = 7.5 |
| | | | Hz, 2H), 1.84 (br t, J = 12.0 Hz, 4H), 1.73 (br d, J = 13.0 Hz, 1H), |
| | | | 1.51-1.37 (m, 4H), 1.32-1.24 (m, 1H) |
| 20 | 475 | 476 | δ 10.31 (s, 1H), 8.77 (s, 1H), 8.63 (d, J = 2.1 Hz, 1H), 8.57-8.50 (m, |
| | | | 2H), 7.94-7.85 (m, 2H), 7.78-7.71 (m, 3H), 7.53-7.48 (m, 2H), |
| | | | 7.43 (d, J = 7.3 Hz, 1H), 3.80-3.71 (m, 2H), 2.45 (t, J = 8.0 Hz, 2H), |
| | | | 1.96 (br t, J = 7.4 Hz, 2H) |
| 21 | 471 | 472 | δ 10.65 (br s, 1H), 8.87-8.68 (m, 3H), 7.88 (dd, J = 8.6, 2.1 Hz, 1H), |
| | | | 7.81 (s, 1H), 7.73 (br d, J = 7.6 Hz, 2H), 7.48 (br t, J = 7.6 Hz, 2H), |
| | | | 7.43-7.25 (m, 3H), 3.88 (s, 3H), 3.76 (br t, J = 6.2 Hz, 2H), 2.49 (br |
| | | | t, J = 8.0 Hz, 2H), 1.92 (br s, 2H) |
| 22 | 475 | 476 | δ 10.31 (s, 1H), 8.77 (s, 1H), 8.68 (br s, 1H), 8.61 (br d, J = 6.1 Hz, |
| | | | 2H), 8.13 (s, 1H), 7.94 (br d, J = 12.3 Hz, 2H), 7.80 (br d, J = 7.5 Hz, |
| | | | 2H), 7.57-7.50 (m, 2H), 7.48 (br d, J = 7.1 Hz, 1H), 3.77 (br t, J = |
| | | | 6.8 Hz, 2H), 2.44 (br t, J = 7.9 Hz, 2H), 2.00-1.91 (m, 2H) |
| 24 | 448 | 449 | δ 9.66 (s, 1H), 8.57 (d, J = 1.6 Hz, 1H), 8.50-8.45 (m, 2H), 8.15 (s, |
| | | | 1H), 4.49-4.35 (m, 2H), 3.79-3.66 (m, 2H), 3.09-2.97 (m, 2H), |
| | | | 2.90-2.81 (m, 1H), 2.42 (t, J = 8.1 Hz, 2H), 2.03-1.93 (m, 3H), |
| | | | 1.89-1.66 (m, 3H) |
| 26 | 448 | 449 | δ 10.21 (s, 1H), 8.71-8.60 (m, 3H), 8.52 (d, J = 2.1 Hz, 1H), 8.41 (s, |
| | | | 1H), 7.76-7.69 (m, 2H), 7.56-7.47 (m, 1H), 7.46-7.40 (m, 1H), |
| | | | 3.84 (br t, J = 7.0 Hz, 4H), 3.26 (br d, J = 12.0 Hz, 2H), 2.94-2.81 |
| | | | (m, 3H), 2.09 (br t, J = 7.5 Hz, 2H), 1.95-1.86 (m, 2H), 1.85-1.72 |
| | | | (m, 2H) |
| 27 | 462 | 463 | δ 10.20 (s, 1H), 8.71-8.62 (m, 3H), 8.51 (d, J = 2.3 Hz, 1H), 7.75- |
| | | | 7.71 (m, 2H), 7.55-7.49 (m, 1H), 7.47-7.42 (m, 1H), 3.84 (t, J = |
| | | | 7.0 Hz, 2H), 3.28-3.23 (m, 2H), 2.87-2.69 (m, 3H), 2.62 (s, 3H), |
| | | | 2.55-2.51 (m, 2H), 2.09 (quin, J = 7.5 Hz, 2H), 2.02-1.94 (m, 2H), |
| | | | 1.90-1.77 (m, 2H) |
| 28 | 490 | 49 | δ 10.19 (s, 1H), 8.67 (d, J = 6.5 Hz, 3H), 8.52 (d, J = 1.8 Hz, 1H), |
| | | | 7.74-7.70 (m, 2H), 7.52-7.43 (m, 2H), 4.56 (br d, J = 13.0 Hz, 1H), |
| | | | 3.95 (br d, J = 13.4 Hz, 1H), 3.84 (t, J = 7.0 Hz, 2H), 3.20-3.12 (m, |
| | | | 1H), 2.94-2.84 (m, 1H), 2.65-2.57 (m, 1H), 2.52 (d, J = 2.0 Hz, |
| | | | 2H), 2.14-2.07 (m, 2H), 2.04 (s, 3H), 1.85 (br t, J = 12.4 Hz, 2H), |
| | | | 1.71-1.59 (m, 1H), 1.55-1.42 (m, 1H) |
| 29, enant A | 448 | 449 | δ 10.07 (s, 1H), 8.66-8.48 (m, 3H), 8.41 (d, J = 1.8 Hz, 1H), 8.35 (br |
| | | | s, 1H), 7.33-7.23 (m, 4H), 7.22-7.16 (m, 1H), 5.68 (br t, J = 7.5 |
| | | | Hz, 1H), 3.95-3.83 (m, 2H), 2.75-2.60 (m, 3H), 2.55 (t, J = 8.1 Hz, |
| | | | 2H), 2.16-2.07 (m, 2H), 2.04-1.91 (m, 1H) |
| 29, enant B | 448 | 449 | δ 8.68 (t, J = 2.2 Hz, 1H), 8.56 (d, J = 2.3 Hz, 1H), 8.47-8.38 (m, |
| | | | 2H), 7.38-7.30 (m, 2H), 7.28-7.15 (m, 3H), 5.75 (dd, J = 8.9, 7.0 |
| | | | Hz, 1H), 3.99 (t, J = 7.1 Hz, 2H), 2.80-2.63 (m, 5 H), 2.25 (quin, J = |
| | | | 7.6 Hz, 2H), 2.19-2.07 (m, 1H) |
| 30 | 448 | 449 | δ 10.20 (s, 1H), 8.73-8.59 (m, 3H), 8.51 (d, J = 2.0 Hz, 1H), 8.35 (s, |
| | | | 1H), 7.82-7.65 (m, 2H), 7.62-7.35 (m, 2H), 3.84 (br t, J = 7.0 Hz, |
| | | | 2H), 3.18 (br d, J = 10.5 Hz, 1H), 3.12 (br d, J = 11.4 Hz, 1H), 2.89 |
| | | | (br d, J = 1.5 Hz, 1H), 2.81 (br d, J = 11.5 Hz, 1H), 2.67 (br d, J = |
| | | | 10.4 Hz, 1H), 2.55-2.52 (m, 2H), 2.10 (td, J = 14.9, 7.5 Hz, 2H), |
| | | | 1.98-1.91 (m, 1H), 1.79 (br d, J = 10.1 Hz, 1H), 1.74-1.59 (m, 2H) |
| 32 | 456 | 457 | δ 10.14 (s, 1H), 8.69 (s, 1H), 8.51 (s, 3H), 8.14 (s, 1H), 7.87 (br dd, |
| | | | J = 16.1, 7.8 Hz, 2H), 7.74 (d, J = 7.4 Hz, 2H), 7.68-7.61 (m, 1H), |
| | | | 7.50 (t, J = 7.6 Hz, 2H), 7.45-7.38 (m, 1H), 3.68 (br t, J = 7.8 Hz, |
| | | | 2H), 3.29-3.25 (m, 2H), 2.71 (s, 3H) |
| 33 | 462 | 463 | δ 10.21 (s, 1H), 8.69 (d, J = 7.1 Hz, 3H), 8.53 (d, J = 1.8 Hz, 1H), |
| | | | 7.78-7.71 (m, 2H), 7.52-7.45 (m, 2H), 3.86 (t, J = 7.0 Hz, 2H), |
| | | | 2.95-2.84 (m, 3H), 2.57-2.53 (m, 2H), 2.26 (s, 3H), 2.16-2.07 (m, |
| | | | 3H), 2.06-1.97 (m, 1H), 1.88 (br d, J = 12.8 Hz, 1H), 1.80-1.72 (m, |
| | | | 1H), 1.71-1.59 (m, 1H), 1.54-1.40 (m, 1H) |
| 34 | 490 | 491 | δ 9.92 (s, 1H), 8.69 (d, J = 1.7 Hz, 1H), 8.63 (s, 1H), 8.58 (t, J = 2.1 |
| | | | Hz, 1H), 8.53 (d, J = 1.7 Hz, 1H), 7.81-7.72 (m, 2H), 7.54-7.45 (m, |
| | | | 2H), 4.59-4.36 (m, 1H), 3.85 (t, J = 7.0 Hz, 3H), 2.86-2.62 (m, |
| | | | 2H), 2.55-2.52 (m, 2H), 2.12 (quin, J = 7.5 Hz, 2H), 2.07-1.98 (m, |
| | | | 5H), 1.79 (br t, J = 10.6 Hz, 2H), 1.55 (br s, 1H) |
| 35 | 496 | 497 | δ 8.79 (br s, 1H), 8.64 (br s, 1H), 8.52 (s, 1H), 8.43 (br s, 1H), 8.12 |
| | | | (br s, 1H), 7.89 (br d, J = 7.4 Hz, 1H), 7.77-7.68 (m, 2H), 7.65 (br d, |
| | | | J = 7.4 Hz, 2H), 7.61-7.55 (m, 1H), 7.47 (br t, J = 7.3 Hz, 2H), 7.42- |
| | | | 7.35 (m, 1H), 3.99 (s, 2H), 3.40-3.15 (m, 4H), 2.76 (s, 2H), 2.34 |
| | | | (s, 3H) |
| 36 | 524 | 525 | δ 8.84 (s, 1H), 8.59 (s, 1H), 8.54 (s, 1H), 8.46 (s, 1H), 8.14 (s, 1H), |
| | | | 7.90 (d, J = 7.8 Hz, 1H), 7.77 (d, J = 7.9 Hz, 1H), 7.67 (d, J = 7.3 Hz, |
| | | | 2H), 7.62-7.58 (m, 1H), 7.57-7.55 (m, 1H), 7.48 (t, J = 7.6 Hz, |
| | | | 2H), 7.43-7.37 (m, 1H), 4.14-4.05 (m, 2H), 4.05-3.94 (m, 4H), |
| | | | 2.80 (s, 2H), 1.89 (s, 3H) |
| 37 | 427 | 428 | δ 9.50 (s, 1H), 8.56 (br d, J = 8.4 Hz, 2H), 8.40 (s, 1H), 8.21 (s, 1H), |
| | | | 7.98 (s, 1H), 4.03-3.69 (m, 6H), 3.55 (br dd, J = 10.4, 7.4 Hz, 2H), |
| | | | 2.76 (dt, J = 13.3, 6.5 Hz, 1H), 2.57-2.51 (m, 2H), 2.46 (br s, 2H), |
| | | | 2.13-2.02 (m, 3H), 1.84 (br dd, J = 11.6, 8.4 Hz, 1H), 1.70 (br s, 4H) |
| 38 | 448 | 449 | δ 8.72-8.68 (m, 2H), 8.63 (d, J = 2.3 Hz, 1H), 8.56 (s, 1H), 7.47 (s, |
| | | | 1H), 7.42-7.34 (m, 2H), 7.16 (dd, J = 7.6, 1.9 Hz, 1H), 3.94 (t, J = |
| | | | 7.1 Hz, 2H), 3.33 (dt, J = 3.1, 1.5 Hz, 2H), 3.26-3.22 (m, 4H), 2.64 |
| | | | (t, J = 8.1 Hz, 2H), 2.27-2.19 (m, 2H), 1.76 (quin, J = 5.6 Hz, 4H), |
| | | | 1.67-1.61 (m, 2H) |
| 39 | 419 | 420 | δ 10.17 (s, 1H), 8.67 (s, 1H), 8.66 (br s, 1H), 8.62 (t, J = 2.1 Hz, 1H), |
| | | | 8.53 (d, J = 2.1 Hz, 1H), 7.71-7.64 (m, 2H), 7.50-7.40 (m, 2H), |
| | | | 3.83 (t, J = 7.0 Hz, 2H), 3.61 (br dd, J = 17.7, 8.8 Hz, 2H), 2.54 (br s, |
| | | | 1H), 2.39-2.19 (m, 2H), 2.20-1.95 (m, 5H), 1.89-1.79 (m, 1H) |
| 40 | 433 | 432 | δ 8.79 (t, J = 2.3 Hz, 1H), 8.65 (d, J = 2.3 Hz, 1H), 8.49 (s, 1H), 8.46 |
| | | | (d, J = 2.3 Hz, 1H), 7.76 (s, 1H), 7.71 (dt, J = 7.1, 1.6 Hz, 1H), 7.47- |
| | | | 7.41 (m, 1H), 7.40-7.37 (m, 2H), 3.90 (t, J = 7.1 Hz, 2H), 3.16- |
| | | | 3.04 (m, 1H), 2.65 (t, J = 8.1 Hz, 2H), 2.27-2.18 (m, 2H), 2.17- |
| | | | 2.08 (m, 2H), 1.89-1.80 (m, 2H), 1.76-1.71 (m, 2H), 1.67-1.60 |
| | | | (m, 2H) |
| 41 | 450 | 45 | δ 9.74 (s, 1H), 8.57-8.48 (m, 2H), 8.43 (d, J = 2.0 Hz, 1H), 8.19 (s, |
| | | | 1H), 7.44-7.29 (m, 5H), 4.62 (dd, J = 10.4, 1.7 Hz, 1H), 4.31 (br d, |
| | | | J = 13.0 Hz, 2H), 4.07 (dd, J = 11.5, 2.0 Hz, 1H), 3.82-3.65 (m, 3H), |
| | | | 3.24-3.15 (m, 1H), 2.99 (dd, J = 13.0, 10.8 Hz, 1H), 2.44-2.31 (m, |
| | | | 2H), 2.05-1.87 (m, 2H) |
| 42 | 434 | 435 | δ 9.56 (s, 1H), 8.55-8.51 (m, 2H), 8.43 (d, J = 1.8 Hz, 1H), 8.02 (s, |
| | | | 1H), 7.38-7.34 (m, 4H), 7.30-7.22 (m, 1H), 4.24-4.17 (m, 1H), |
| | | | 4.10-4.00 (m, 1H), 3.94-3.86 (m, 1H), 3.72-3.66 (m, 3H), 2.38- |
| | | | 2.27 (m, 4H), 2.15-2.03 (m, 1H), 1.90-1.81 (m, 2H) |
| 43 | 448 | 449 | δ 9.64 (s, 1H), 8.56 (d, J = 1.8 Hz, 1H), 8.46 (q, J = 2.3 Hz, 2H), 8.13 |
| | | | (s, 1H), 7.52-7.46 (m, 1H), 7.40-7.37 (m, 2H), 7.32 (br d, J = 7.0 |
| | | | Hz, 2H), 4.48-4.33 (m, 2H), 3.72 (dt, J = 11.4, 6.9 Hz, 2H), 3.07- |
| | | | 2.96 (m, 2H), 2.89-2.79 (m, 1H), 2.41 (t, J = 8.1 Hz, 2H), 2.02- |
| | | | 1.91 (m, 3H), 1.88-1.68 (m, 3H) |
| 44 | 448 | 449 | δ 9.64 (s, 1H), 8.56 (d, J = 1.8 Hz, 1H), 8.46 (q, J = 2.3 Hz, 2H), 8.13 |
| | | | (s, 1H), 7.52-7.46 (m, 1H), 7.40-7.37 (m, 2H), 7.32 (br d, J = 7.0 |
| | | | Hz, 2H), 4.48-4.33 (m, 2H), 3.72 (dt, J = 11.4, 6.9 Hz, 2H), 3.07- |
| | | | 2.96 (m, 2H), 2.89-2.79 (m, 1H), 2.41 (t, J = 8.1 Hz, 2H), 2.02- |
| | | | 1.91 (m, 3H), 1.88-1.68 (m, 3H) |
| 45 | 440 | 441 | δ 9.99 (s, 1H), 8.84 (s, 1H), 8.76-8.63 (m, 2H), 4.01-3.89 (m, 2H), |
| | | | 3.87 (t, J = 7.1 Hz, 2H), 3.74-3.65 (m, 1H), 3.35 (br t, J = 10.5 Hz, |
| | | | 1H), 2.55 (t, J = 8.1 Hz, 2H), 2.52-2.18 (m, 1H), 2.16-2.04 (m, |
| | | | 2H), 1.96-1.84 (m, 1H), 1.78 (br d, J = 12.6 Hz, 1H), 1.72-1.60 (m, |
| | | | 4H), 1.56-1.45 (m, 1H), 1.27-1.13 (m, 4H), 1.00 (quin, J = 11.7 |
| | | | Hz, 2H) |
| 46 | 434 | 433 | δ 10.14 (s, 1H), 8.72 (s, 1H), 8.64 (s, 1H), 8.55 (s, 2H), 7.31 (t, J = |
| | | | 7.9 Hz, 1H), 7.03 (d, J = 7.6 Hz, 1H), 6.9 (s, 1H), 6.70 (dd, J = 8.1, |
| | | | 2.1 Hz, 1H), 3.82 (t, J = 7.0 Hz, 2H), 3.30-3.24 (m, 6H), 2.12-2.04 |
| | | | (m, 2H), 2.02-1.93 (m, 4H) |
| 47 | 452 | 453 | δ 10.02 (s, 1H), 9.02-8.98 (m, 1H), 8.79 (s, 1H), 8.56-8.53 (m, |
| | | | 2H), 8.38 (s, 1H), 8.32 (d, J = 2.1 Hz, 1H), 8.19-8.16 (m, 1H), 4.61- |
| | | | 4.45 (m, 2H), 3.92-3.88 (m, 2H), 3.36-3.30 (m, 2H), 2.78 (br s, |
| | | | 2H), 2.64 (s, 3H), 2.52-2.49 (m, 1H), 2.22 (br d, J = 3.0 Hz, 4H), |
| | | | 2.13-2.08 (m, 2H) |
| 48 | 480 | 481 | δ 10.02 (s, 1H), 9.08-9.02 (m, 1H), 8.80 (s, 1H), 8.56 (s, 1H), 8.54- |
| | | | 8.52 (m, 1H), 8.37 (s, 1H), 8.31 (d, J = 2.3 Hz, 1H), 4.61-4.51 (m, |
| | | | 2H), 4.00-3.95 (m, 1H), 3.91 (t, J = 7.0 Hz, 2H), 3.26-3.17 (m, |
| | | | 1H), 2.76-2.67 (m, 1H), 2.54 (s, 2H), 2.12 (s, 4H), 2.06 (s, 3H), 2.04- |
| | | | 1.96 (m, 1H), 1.90-1.82 (m, 1H) |
| 49 | 431 | 432 | δ 9.81-9.57 (m, 1H), 8.43-8.31 (m, 1H), 8.30-8.18 (m, 1H), 8.04 |
| | | | (br s, 1H), 7.99-7.84 (m, 2H), 7.71-7.57 (m, 1H), 7.41 (br s, 2H), |
| | | | 7.34 (br d, J = 6.4 Hz, 1H), 4.04-3.77 (m, 2H), 2.82-2.62 (m, 2H), |
| | | | 2.32-2.03 (m, 2H) |
| 50 | 395 | 396 | δ 13.21 (br s, 1H), 10.09 (br s, 1H), 8.85 (br s, 1H), 8.60 (br s, 2H), |
| | | | 8.47 (br s, 1H), 6.77 (br s, 1H), 3.90 (t, J = 7.0 Hz, 2H), 2.58-2.53 |
| | | | (m, 2H), 2.12 (quin, J = 7.5 Hz, 2H), 2.04-1.92 (m, 1 H), 0.98 (br s, |
| | | | 2H), 0.78 (br s, 2H) |
| 90 | 369 | 370 | δ 10.02 (s, 1H), 8.93 (br s, 1H), 8.77 (s, 1H), 8.58 (d, J = 2.3 Hz, 1H), |
| | | | 8.56 (s, 1H), 8.37 (d, J = 2.1 Hz, 1H), 8.32 (s, 1H), 3.97 (s, 3H), 3.91 |
| | | | (t, J = 7.0 Hz, 2H), 2.57 (t, J = 8.1 Hz, 2H), 2.12 (quin, J = 7.5 Hz, 2H) |
| 92 | 399 | 400 | δ 10.27 (s, 1H), 8.72 (s, 1H), 8.66 (d, J = 2.0 Hz, 1H), 8.61 (d, J = 2.0 |
| | | | Hz, 1H), 8.58 (d, J = 2.1 Hz, 1H), 7.92 (d, J = 1.6 Hz, 1H), 7.86 (d, |
| | | | J = 7.5 Hz, 1H), 7.67-7.63 (m, 1H), 7.63-7.57 (m, 1H), 3.86 (t, J = |
| | | | 7.0 Hz, 2H), 2.55-2.53 (m, 2H), 2.11 (t, J = 7.5 Hz, 2H) |
| 93 | 399 | 400 | δ 10.24 (s, 1H), 8.71 (s, 1H), 8.66 (s, 2H), 8.52 (d, J = 1.0 Hz, 1H), |
| | | | 7.96-7.91 (m, 2H), 7.64 (d, J = 8.5 Hz, 2H), 3.85 (t, J = 7.0 Hz, 2H), |
| | | | 2.54 (br s, 2H), 2.11 (t, J = 7.5 Hz, 2H) |
| 94 | 379 | 380 | δ 10.17 (s, 1H), 8.69-8.66 (m, 2H), 8.63 (t, J = 2.2 Hz, 1H), 8.54 (d, |
| | | | J = 2.3 Hz, 1H), 7.82 (d, J = 8.2 Hz, 2H), 7.37 (d, J = 8.0 Hz, 2H), |
| | | | 3.85 (t, J = 7.0 Hz, 2H), 2.56-2.52 (m, 2H), 2.41 (s, 3H), 2.11 (quin, |
| | | | J = 7.5 Hz, 2H) |
| 95 | 399 | 400 | δ 10.34 (s, 1H), 8.77 (s, 1H), 8.71 (s, 1H), 8.57 (d, J = 1.8 Hz, 1H), |
| | | | 8.51 (d, J = 1.8 Hz, 1H), 7.69-7.66 (m, 1H), 7.62-7.59 (m, 2H), |
| | | | 7.58-7.56 (m, 1H), 3.84 (t, J = 7.0 Hz, 2H), 2.56 (br s, 2H), 2.11 (t, |
| | | | J = 7.5 Hz, 2H) |
| 96 | 372 | 373 | δ 9.61 (s, 1H), 8.58 (d, J = 2.2 Hz, 1H), 8.48-8.46 (m, 1H), 8.43 (d, |
| | | | J = 2.4 Hz, 1H), 8.08 (s, 1H), 3.83-3.80 (m, 2H), 3.61-3.57 (m, |
| | | | 4H), 2.11 (s, 3H), 1.60 (br s, 7H) |
| 97 | 371 | 372 | δ 9.99 (s, 1H), 8.65-8.60 (m, 2H), 8.55 (d, J = 2.3 Hz, 1H), 8.49 (d, |
| | | | J = 0.8 Hz, 1H), 3.88 (t, J = 7.0 Hz, 2H), 3.08-2.98 (m, 1H), 2.16- |
| | | | 2.07 (m, 3H), 1.83 (br s, 5H), 1.74 (br d, J = 12.3 Hz, 1H), 1.67-1.55 |
| | | | (m, 3H), 1.44-1.35 (m, 2H) |
| 98 | 407 | 408 | δ 10.44 (br s, 1H), 8.82-8.74 (m, 3H), 8.67 (s, 1H), 7.76-7.70 (m, |
| | | | 2H), 7.53-7.47 (m, 2H), 3.89 (t, J = 7.0 Hz, 2H), 3.08-2.99 (m, |
| | | | 1H), 2.60-2.56 (m, 2H), 2.14 (quin, J = 7.5 Hz, 2H), 1.29 (d, J = 6.8 |
| | | | Hz, 6H) |
| 99 | 388 | 389 | δ 9.63 (s, 1H), 8.60 (d, J = 2.1 Hz, 1H), 8.51-8.43 (m, 2H), 8.10 (s, |
| | | | 1H), 4.89 (br d, J = 3.4 Hz, 1H), 4.12 (br dd, J = 12.6, 3.6 Hz, 1H), |
| | | | 3.96 (br d, J = 12.8 Hz, 1H), 3.86 (t, J = 7.4 Hz, 2H), 3.66-3.40 (m, |
| | | | 2H), 3.12 (ddd, J = 12.9, 10.3, 2.7 Hz, 1H), 3.00 (dd, J = 12.6, 8.8 |
| | | | Hz, 1H), 2.09 (quin, J = 7.5 Hz, 2H), 1.96-1.88 (m, 1H), 1.85-1.78 |
| | | | (m, 1H), 1.58-1.46 (m, 1H), 1.45-1.35 (m, 1H) |
| 100 | 386 | 387 | δ 9.64-9.60 (m, 1H), 8.62-8.58 (m, 1H), 8.49 (t, J = 2.2 Hz, 1H), |
| | | | 8.45 (d, J = 2.2 Hz, 1H), 8.10 (s, 1H), 4.27-4.13 (m, 2H), 3.85 (t, J = |
| | | | 7.0 Hz, 2H), 2.96-2.88 (m, 1H), 2.64 (dd, J = 12.8, 10.7 Hz, 1H), |
| | | | 2.48 (br s, 1H), 2.11-2.04 (m, 2H), 1.82-1.79 (m, 1H), 1.78 (br d, |
| | | | J = 12.6 Hz, 1H), 1.74-1.67 (m, 1H), 1.61-1.53 (m, 1H), 1.23-1.13 |
| | | | (m, 1H), 0.90 (d, J = 6.7 Hz, 3H), |
| 101 | 440 | 441 | δ 9.73-9.68 (m, 1H), 8.58 (s, 1H), 8.52 (s, 1H), 8.45 (d, J = 1.8 Hz, |
| | | | 1H), 8.18 (s, 1H), 4.39 (br d, J = 12.2 Hz, 1H), 4.27 (br d, J = 13.2 |
| | | | Hz, 1H), 3.85 (t, J = 6.9 Hz, 2H), 3.07-2.97 (m, 2H), 2.70-2.57 (m, |
| | | | 2H), 2.48 (s, 1H), 2.05-2.12 (m, 2H), 2.00 (br d, J = 10.4 Hz, 1H), |
| | | | 1.86-1.79 (m, 1H), 1.66-1.53 (m, 2H) |
| 102 | 386 | 387 | δ 9.63 (s, 1H), 8.61 (d, J = 2.2 Hz, 1H), 8.49 (t, J = 2.3 Hz, 1H), 8.45 |
| | | | (d, J = 2.3 Hz, 1H), 8.10 (s, 1H), 4.31 (br d, J = 13.0 Hz, 2H), 3.87- |
| | | | 3.83 (m, 2H), 2.94 (dt, J = 12.6, 1.8 Hz, 2H), 2.10 (quin, J = 7.5 Hz, |
| | | | 3H), 1.75-1.60 (m, 4H), 1.26-1.16 (m, 2H), 0.94 (d, J = 6.4 Hz, 3H) |
| 103 | 382 | 383 | δ 9.56 (s, 1H), 8.71 (d, J = 1.8 Hz, 1H), 8.52 (s, 1H), 8.37 (d, J = 1.8 |
| | | | Hz, 1H), 8.30 (s, 1H), 8.16 (s, 1H), 7.57 (br dd, J = 8.5, 5.5 Hz, 2H), |
| | | | 7.36 (br t, J = 8.8 Hz, 2H), 6.89 (s, 1H), 3.87 (br t, J = 7.0 Hz, 2H), |
| | | | 2.56-2.51 (m, 2H), 2.10 (quin, J = 7.5 Hz, 2H) |
| 104 | 400 | 401 | δ 9.59 (s, 1H), 8.72 (d, J = 2.1 Hz, 1H), 8.51 (t, J = 2.3 Hz, 1H), 8.38 |
| | | | (d, J = 2.3 Hz, 1H), 8.34 (s, 1H), 7.54 (td, J = 8.5, 6.6 Hz, 1H), 7.46 |
| | | | (td, J = 9.8, 2.5 Hz, 1H), 7.27 (td, J = 8.4, 2.3 Hz, 1H), 6.88 (s, 1H), |
| | | | 3.87 (t, J = 7.1 Hz, 2H), 2.57-2.51 (m, 2H), 2.10 (quin, J = 7.6 Hz, 2H) |
| 105 | 380 | 381 | δ 10.31 (s, 1H), 8.75 (s, 2H), 8.64 (d, J = 5.1 Hz, 1H), 8.60 (d, J = 2.1 |
| | | | Hz, 1H), 8.52 (d, J = 2.1 Hz, 1H), 7.70 (s, 1H), 7.62 (d, J = 5.0 Hz, |
| | | | 1H), 3.85 (t, J = 7.1 Hz, 2H), 2.59 (s, 3H), 2.56-2.52 (m, 2H), 2.15- |
| | | | 2.14 (m, 1H), 2.10-2.09 (m, 1H) |
| 106 | 386 | 387 | δ 9.58 (s, 1H), 8.63 (d, J = 2.1 Hz, 1H), 8.48-8.42 (m, 2H), 8.09 (s, |
| | | | 1H), 4.65 (br d, J = 3.1 Hz, 1H), 4.11-4.03 (m, 1H), 3.85 (t, J = 7.0 |
| | | | Hz, 2H), 3.14 (br s, 1H), 2.53-2.52 (m, 1 H), 2.50-2.48 (m, 1H), |
| | | | 2.10 (t, J = 7.4 Hz, 2H), 1.69 (br s, 3H), 1.56 (br s, 3H), 1.25 (d, J = |
| | | | 6.8 Hz, 3H) |
| 107 | 374 | 375 | δ 8.73 (t, J = 2.3 Hz, 1H), 8.59 (d, J = 2.1 Hz, 1H), 8.28 (d, J = 2.0 |
| | | | Hz, 1H), 8.06 (s, 1H), 8.04-8.03 (m, 1H), 7.23 (s, 1H), 3.92 (t, J = |
| | | | 7.1 Hz, 2H), 3.85-3.80 (m, 4H), 3.78-3.73 (m, 4H), 2.63 (t, J = 8.1 |
| | | | Hz, 2H), 2.30-2.18 (m, 2H) |
| 108 | 401 | 402 | δ = 10.28 (s, 1H), 8.73 (s, 1H), 8.65 (d, J = 1.5 Hz, 1H), 8.53 (s, 2H), |
| | | | 7.78-7.70 (m, 1H), 7.48 (dt, J = 9.9, 2.3 Hz, 1H), 7.30 (dt, J = 8.3, |
| | | | 2.3 Hz, 1H), 3.82 (t, J = 7.0 Hz, 2H), 2.53 (s, 2H), 2.09 (t, J = 7.6 Hz, 2H) |
| 109 | 471 | 472 | δ 9.66 (s, 1H), 8.59 (d, J = 2.1 Hz, 1H), 8.50 (d, J = 2.3 Hz, 1H), 8.45 |
| | | | (t, J = 2.3 Hz, 1H), 8.13 (s, 1H), 7.80 (t, J = 5.6 Hz, 1H), 4.27 (br d, |
| | | | J = 13.0 Hz, 2H), 3.83 (t, J = 7.0 Hz, 2H), 3.10-2.87 (m, 5H), 2.45- |
| | | | 2.36 (m, 1H), 2.10 (quin, J = 7.5 Hz, 2H), 1.92-1.84 (m, 1H), 1.76 |
| | | | (br d, J = 12.0 Hz, 1H), 1.70-1.49 (m, 2H), 1.42-1.19 (m, 5H), 0.86 |
| | | | (t, J = 7.3 Hz, 3H) |
| 110 | 471 | 472 | δ 9.65 (s, 1H), 8.61 (d, J = 1.5 Hz, 1H), 8.50-8.45 (m, 2H), 8.12 (s, |
| | | | 1H), 7.78 (br d, J = 7.3 Hz, 1H), 4.05-3.99 (m, 1H), 3.92 (br d, J = |
| | | | 12.8 Hz, 1H), 3.88-3.82 (m, 2H), 3.78-3.71 (m, 1H), 3.22-3.07 |
| | | | (m, 2H), 2.54 (br s, 1H), 2.15-2.01 (m, 5H), 1.85 (br s, 2H), 1.68- |
| | | | 1.56 (m, 1H), 1.50-1.40 (m, 3H), 1.23 (qd, J = 14.8, 7.3 Hz, 2H), |
| | | | 0.85 (t, J = 7.3 Hz, 3H) |
| 111 | 408 | 409 | δ 8.81 (t, J = 2.3 Hz, 1H), 8.63 (d, J = 4.8 Hz, 1H), 8.56 (d, J = 1.9 |
| | | | Hz, 1H), 8.46 (s, 1H), 8.30 (d, J = 1.9 Hz, 1H), 7.58 (s, 1H), 7.53 (dd, |
| | | | J = 5.1, 1.6 Hz, 1H), 7.44 (s, 1H), 3.83 (t, J = 7.1 Hz, 2H), 3.12 (td, |
| | | | J = 13.9, 6.9 Hz, 1H), 2.57 (t, J = 8.1 Hz, 2H), 2.14 (quin, J = 7.6 Hz, |
| | | | 2H), 1.30 (d, J = 6.9 Hz, 6H) |
| 112 | 417 | 418 | δ 2.13 (t, J = 7.6 Hz, 2H), 2.57 (t, J = 8.1 Hz, 2H), 3.81 (t, J = 7.1 Hz, |
| | | | 2H), 7.03-7.09 (m, 1H), 7.18 (d, J = 2.5 Hz, 1H), 7.20 (d, J = 2.6 |
| | | | Hz, 1H), 7.34 (dd, J = 8.6, 5.9 Hz, 1H), 7.43 (s, 1H), 8.34 (d, J = 2.1 |
| | | | Hz, 1H), 8.45 (s, 1H), 8.60 (d, J = 2.1 Hz, 1H), 8.66 (t, J = 2.3 Hz, 1H) |
| 113 | 397 | 398 | δ 8.72 (t, J = 2.1 Hz, 1H), 8.65 (d, J = 2.0 Hz, 1H), 8.49 (s, 1H), 8.37 |
| | | | (d, J = 2.0 Hz, 1H), 7.40 (s, 1H), 7.29-7.26 (m, 1H), 7.02-6.95 (m, |
| | | | 2H), 3.86 (t, J = 7.0 Hz, 2H), 2.62 (t, J = 8.1 Hz, 2H), 2.25 (s, 3H), |
| | | | 2.18 (quin, J = 7.5 Hz, 2H) |
| 114 | 402 | 403 | δ 8.77 (t, J = 2.3 Hz, 1H), 8.55 (d, J = 2.3 Hz, 1H), 8.33 (d, J = 2.3 |
| | | | Hz, 1H), 8.06 (s, 1H), 7.10 (s, 1H), 4.32 (br d, J = 13.0 Hz, 2H), 3.94 |
| | | | (t, J = 7.1 Hz, 2H), 3.83-3.73 (m, 2H), 2.75-2.69 (m, 2H), 2.68- |
| | | | 2.63 (m, 2H), 2.24 (quin, J = 7.6 Hz, 2H), 1.26 (d, J = 6.3 Hz, 6H) |
| 115 | 400 | 401 | δ 9.65 (s, 1H), 8.60 (d, J = 2.0 Hz, 1H), 8.51 (t, J = 2.2 Hz, 1H), 8.45 |
| | | | (d, J = 2.2 Hz, 1H), 8.11 (s, 1H), 4.28 (br dd, J = 11.3, 1.3 Hz, 2H), |
| | | | 3.87 (t, J = 7.0 Hz, 2H), 2.49-2.39 (m, 4H), 2.09 (quin, J = 7.51 Hz, |
| | | | 3H), 1.81 (br d, J = 12.3 Hz, 1H), 1.73-1.65 (m, 2H), 0.90 (s, 3H), |
| | | | 0.88 (s, 3H) |
| 116 | 379 | 380 | δ 8.75-8.71 (m, 1H), 8.67 (d, J = 2.0 Hz, 1H), 8.51 (s, 1H), 8.43 (d, |
| | | | J = 2.0 Hz, 1H), 7.42-7.35 (m, 2H), 7.34-7.28 (m, 3H), 3.88 (t, J = |
| | | | 7.0 Hz, 2H), 2.64 (t, J = 8.1 Hz, 2H), 2.27 (s, 3H), 2.24-2.15 (m, 2H) |
| 117 | 364 | 365 | δ 9.54 (s, 1H), 8.72 (d, J = 2.1 Hz, 1H), 8.53 (t, J = 2.2 Hz, 1H), 8.37 |
| | | | (d, J = 2.1 Hz, 1H), 8.31 (s, 1H), 7.56-7.46 (m, 5H), 6.90 (s, 1H), |
| | | | 3.87 (t, J = 7.1 Hz, 2H), 2.55-2.51 (m, 2H), 2.10 (quin, J = 7.5 Hz, 2H) |
| 118 | 383 | 384 | δ = 10.22 (s, 1H), 8.69 (s, 1H), 8.66-8.64 (m, 2H), 8.52 (d, J = 2.0 |
| | | | Hz, 1H), 7.97 (dd, J = 8.6, 5.4 Hz, 2H), 7.40 (t, J = 8.9 Hz, 2H), 3.84 |
| | | | (t, J = 7.1 Hz, 2H), 2.55-2.53 (m, 2H), 2.10 (t, J = 7.7 Hz, 2H) |
| 119 | 440 | 44 | δ 8.56 (t, J = 2.3 Hz, 1H), 8.50 (d, J = 2.2 Hz, 1H), 8.27 (d, J = 2.2 |
| | | | Hz, 1H), 8.02 (s, 1H), 7.03 (s, 1H), 5.27-5.17 (m, 1H), 4.06 (br d, |
| | | | J = 13.1 Hz, 1H), 3.84 (t, J = 7.0 Hz, 2H), 3.32 (br t, J = 12.8 Hz, 1H), |
| | | | 2.56 (t, J = 8.1 Hz, 2H), 2.15 (quin, J = 7.5 Hz, 2H), 2.03 (br d, J = |
| | | | 14.2 Hz, 1H), 1.91-1.80 (m, 1H), 1.78-1.65 (m, 4H) |
| 120 | 435 | 436 | δ 10.08 (br s, 1 H), 8.84 (s, 1 H), 8.70 (br d, J = 9.9 Hz, 2 H), 8.63 (br |
| | | | d, J = 4.9 Hz, 1 H), 8.10 (s, 1 H), 7.95 (br t, J = 7.7 Hz, 1 H), 7.56 (d, |
| | | | J = 7.9 Hz, 1 H), 7.47-7.39 (m, 1 H), 4.27 (br dd, J = 10.6, 7.9 Hz, 1 |
| | | | H), 4.10-3.88 (m, 3 H), 3.80 (br t, J = 7.0 Hz, 2 H), 3.74-3.65 (m, |
| | | | 1 H), 2.47 (br d, J = 8.4 Hz, 2 H), 2.38 (ddq, J = 12.1, 6.14, 3.19, |
| | | | 3.19, 3.19 Hz, 1 H), 2.30-2.13 (m, 1 H), 2.00 (quin, J = 7.5 Hz, 2 H) |
| 121 | 441 | 442 | δ 8.75-8.67 (m, 2 H), 8.48 (d, J = 1.4 Hz, 1 H), 8.10 (s, 1 H) 4.26- |
| | | | 4.15 (m, 1 H), 4.08-4.00 (m, 1 H), 3.99-3.91 (m, 2 H), 3.90-3.84 |
| | | | (m, 2 H), 3.83-3.75 (m, 1 H), 3.50 (br d, J = 12.3 Hz, 1 H), 3.44- |
| | | | 3.39 (m, 1 H), 3.09-2.95 (m, 2 H), 2.56-2.51 (m, 2 H), 2.47-2.37 |
| | | | (m, 1 H), 2.22-2.05 (m, 3 H), 1.87 (br d, J = 13.0 Hz, 2 H), 1.76- |
| | | | 1.61 (m, 3 H), 1.51-1.37 (m, 1 H) |
| 122 | 420 | 421 | δ 10.15 (s, 1 H), 8.69 (d, J = 1.5 Hz, 1 H), 8.64 (s, 1 H), 8.55 (s, 2 H), |
| | | | 7.32 (t, J = 7.8 Hz, 1 H), 7.13 (d, J = 7.7 Hz, 1 H), 6.81 (t, J = 1.8 Hz, |
| | | | 1 H), 6.58 (dd, J = 8.1, 1.56 Hz, 1 H), 3.84 (q, J = 7.3 Hz, 6 H), 2.54- |
| | | | 2.51 (m, 2 H), 2.33 (quin, J = 7.2 Hz, 2 H), 2.14-2.06 (m, 2 H) |
| 124 | 449 | 450 | δ 9.64 (s, 1H), 8.57 (s, 1H), 8.53 (br d, J = 4.0 Hz, 1H), 8.46 (s, 2H), |
| | | | 8.13 (s, 1H), 7.74 (dt, J = 7.7, 1.8 Hz, 1H), 7.31 (d, J = 7.9 Hz, 1H), |
| | | | 7.28-7.22 (m, 1H), 4.55-4.39 (m, 2H), 3.85-3.67 (m, 2H), 3.22- |
| | | | 3.13 (m, 1H), 3.06-2.95 (m, 2H), 2.43 (t, J = 8.0 Hz, 2H), 2.06- |
| | | | 1.97 (m, 3H), 1.93-1.79 (m, 2H), 1.78-1.66 (m, 1H) |
| 125 | 449 | 450 | δ 9.67 (s, 1H), 8.61 (s, 1H), 8.57 (br d, J = 4.3 Hz, 1H), 8.50 (s, 2H), |
| | | | 8.17 (s, 1H), 7.81-7.75 (m, 1H), 7.35 (d, J = 7.9 Hz, 1H), 7.30 (dd, |
| | | | J = 6.8, 5.0 Hz, 1H), 4.56-4.45 (m, 2H), 3.82-3.74 (m, 2H), 3.25- |
| | | | 3.18 (m, 1H), 3.08-3.02 (m, 2H), 2.49-2.44 (m, 2H), 2.08-2.02 |
| | | | (m, 3H), 1.93-1.84 (m, 2H), 1.81-1.72 (m, 1H) |
| 126 | 412 | 413 | δ 9.61 (s, 1H), 8.60 (d, J = 2.0 Hz, 1H), 8.50-8.45 (m, 2H), 8.09 (s, |
| | | | 1H), 4.28-4.15 (m, 2H), 3.85 (t, J = 7.0 Hz, 2H), 3.01-2.82 (m, |
| | | | 2H), 2.52 (br s, 1H), 2.48 (br s, 1H), 2.08 (quin, J = 7.5 Hz, 2H), 1.92- |
| | | | 1.70 (m, 2H), 1.58-1.26 (m, 2H), 0.89-0.50 (m, 2H), 0.45-0.32 |
| | | | (m, 2H), 0.18-0.04 (m, 2H) |
| 127 | 420 | 421 | δ 9.88 (s, 1H), 8.58 (d, J = 2.1 Hz, 1H), 8.53-8.48 (m, 2H), 8.32 (s, |
| | | | 1H), 7.20-7.14 (m, 1H), 7.08-7.02 (m, 1H), 6.98-6.92 (m, 1H), |
| | | | 6.76 (dd, J = 8.1, 0.8 Hz, 1H), 3.85 (t, J = 6.2 Hz, 2H), 3.72 (t, J = 7.0 |
| | | | Hz, 2H), 2.79 (t, J = 6.6 Hz, 2H), 2.48 (s, 2H), 2.11-2.05 (m, 2H), |
| | | | 1.98 (quin, J = 6.4 Hz, 2H) |
| 128 | 420 | 421 | δ 10.17 (s, 1H), 8.91-8.87 (m, 1H), 8.83-8.80 (m, 1H), 8.72 (d, J = |
| | | | 2.1 Hz, 1H), 8.25 (s, 1H), 7.26-7.22 (m, 4H), 4.85 (s, 2H), 3.94 (t, |
| | | | J = 6.7 Hz, 4H), 3.02 (t, J = 5.7 Hz, 2H), 2.61 (t, J = 8.1 Hz, 2H), 2.21- |
| | | | 2.12 (m, 2H) |
| 129 | 406 | 407 | δ 9.79 (s, 1H), 8.59 (d, J = 2.3 Hz, 1H), 8.50 (d, J = 2.3 Hz, 1H), 8.40 |
| | | | (t, J = 2.3 Hz, 1H), 8.35 (s, 1H), 7.34 (br d, J = 8.1 Hz, 1H), 7.29 (d, |
| | | | J = 7.3 Hz, 1H), 7.08 (t, J = 7.5 Hz, 1H), 6.99-6.94 (m, 1H), 4.31 (t, |
| | | | J = 8.3 Hz, 2H), 3.62 (t, J = 6.9 Hz, 2H), 3.16 (t, J = 8.1 Hz, 2H), 2.50- |
| | | | 2.45 (m, 2H), 2.09-1.97 (m, 2H) |
| 130 | 406 | 407 | δ 9.66 (s, 1H), 8.77 (s, 1H), 8.55 (d, J = 2.0 Hz, 1H), 8.45 (d, J = 2.1 |
| | | | Hz, 1H), 8.09 (s, 1H), 7.43-7.39 (m, 2H), 7.38-7.34 (m, 2H), 5.25 |
| | | | (s, 4H), 3.95 (t, J = 7.0 Hz, 2H), 2.62-2.55 (m, 2H), 2.16 (br t, J = |
| | | | 7.5 Hz, 2H) |
| 131 | 440 | 441 | δ10.05 (s, 1H), 8.90 (br s, 1H), 8.79-8.59 (m, 2H), 8.17 (s, 1H), 4.40- |
| | | | 4.20 (m, 2H), 3.88 (t, J = 7.1 Hz, 2H), 3.03-2.88 (m, 1H), 2.71 (dd, |
| | | | J = 12.9, 10.6 Hz, 1H), 2.62-2.52 (m, 2H), 2.13 (quin, J = 7.5 Hz, |
| | | | 2H), 1.97-1.83 (m, 1H), 1.83-1.67 (m, 3H), 1.62-1.43 (m, 6H), |
| | | | 1.36 (br d, J = 9.6 Hz, 1H), 1.27-1.07 (m, 3H) |
| 132 | 524 | 525 | δ 8.85 (t, J = 2.2 Hz, 1H), 8.60 (d, J = 2.4 Hz, 1H), 8.55-8.49 (m, |
| | | | 2H), 8.13 (s, 1H), 7.93 (d, J = 7.8 Hz, 1H), 7.76 (d, J = 7.8 Hz, 1H), |
| | | | 7.66 (d, J = 7.3 Hz, 2H), 7.63-7.57 (m, 1H), 7.52-7.46 (m, 2H), |
| | | | 7.44-7.38 (m, 1H), 7.36 (s, 1H), 3.70 (t, J = 6.9 Hz, 2H), 3.06 (br s, |
| | | | 2H), 2.56-2.42 (m, 5H), 2.06-2.00 (m, 2H), 1.91 (br t, J = 6.9 Hz, |
| | | | 2H), 1.76-1.65 (m, 2H) |
| 133 | 528 | 529 | δ 8.89 (s, 1H), 8.60 (d, J = 2.0 Hz, 1H), 8.54-8.52 (m, 2H), 8.14 (s, |
| | | | 1H), 7.93 (br d, J = 7.7 Hz, 1H), 7.76 (br d, J = 7.7 Hz, 1H), 7.67 (d, |
| | | | J = 7.3 Hz, 2H), 7.61-7.57 (m, 1H), 7.49 (t, J = 7.5 Hz, 2H), 7.42- |
| | | | 7.38 (m, 2H), 4.30-4.25 (m, 1H), 3.88-3.84 (m, 1H), 3.72 (br t, J = |
| | | | 6.9 Hz, 2H), 3.29-3.22 (m, 1H), 3.16-3.09 (m, 1H), 2.13 (s, 3H), |
| | | | 1.99-1.82 (m, 5H), 1.51-1.45 (m, 2H) |
| 134 | 451 | 452 | δ 9.73 (s, 1H), 8.57-8.51 (m, 3H), 8.44 (d, J = 1.5 Hz, 1H), 8.20 (s, |
| | | | 1H), 7.85 (dt, J = 7.7, 1.8 Hz, 1H), 7.52 (d, J = 7.9 Hz, 1H), 7.39- |
| | | | 7.32 (m, 1H), 4.72-4.61 (m, 2H), 4.30 (br d, J = 13.1 Hz, 1H), 4.13 |
| | | | (dd, J = 11.4, 1.6 Hz, 1H), 3.86 (dt, J = 11.7, 2.3 Hz, 1H), 3.82-3.70 |
| | | | (m, 2H), 3.24-3.15 (m, 1H), 3.05 (dd, J = 12.4, 9.9 Hz, 1H), 2.46- |
| | | | 2.37 (m, 2H), 1.98 (br t, J = 7.1 Hz, 2H) |
| 135 | 440 | 441 | δ 10.04 (s, 1H), 8.66 (s, 1H), 8.59 (br s, 1H), 8.51 (s, 2H), 8.18 (br s, |
| | | | 1H), 3.88 (br t, J = 7.0 Hz, 2H), 3.33-3.19 (m, 2H), 3.10 (br dd, J = |
| | | | 18.7, 11.3 Hz, 2H), 2.80-2.60 (m, 1H), 2.38 (br t, J = 11.0 Hz, 1H), |
| | | | 2.25-2.05 (m, 3H), 1.92-1.73 (m, 5H), 1.62 (br d, J = 8.4 Hz, 4H), |
| | | | 1.52-1.29 (m, 4H) |
| 136 | 426 | 427 | δ 9.90 (s, 1 H), 8.79 (s, 1 H), 8.69 (s, 1 H), 8.63 (s, 1 H), 8.04 (s, 1 |
| | | | H), 3.94-3.90 (m, 1 H), 3.94-3.87 (m, 5 H), 3.37 (br s, 2 H), 2.59- |
| | | | 2.52 (m, 2 H), 2.19-1.91 (m, 4 H), 1.86-1.42 (m, 8 H), 1.30-1.11 |
| | | | (m, 2 H) |
| 137 | 409 | 410 | δ 10.02 (s, 1H), 9.00 (br s, 1H), 8.80 (s, 1H), 8.56 (s, 1H), 8.54 (d, J = |
| | | | 2.3 Hz, 1H), 8.37 (s, 1H), 8.33 (d, J = 2.3 Hz, 1H), 4.99 (quin, J = 8.4 |
| | | | Hz, 1H), 3.91 (t, J = 7.1 Hz, 2H), 2.60-2.54 (m, 4H), 2.46-2.38 (m, |
| | | | 2H), 2.16-2.07 (m, 2H), 1.87-1.77 (m, 2H) |
| 138 | 423 | 424 | δ 10.01 (s, 1H), 8.97 (br s, 1H), 8.74 (s, 1H), 8.57-8.53 (m, 2H), |
| | | | 8.35 (s, 2H), 4.85 (quin, J = 7.2 Hz, 1H), 3.91 (t, J = 7.0 Hz, 2H), |
| | | | 2.55 (t, J = 8.1 Hz, 2H), 2.18-2.08 (m, 4H), 2.06-1.95 (m, 2H), |
| | | | 1.90-1.79 (m, 2H), 1.68 (br dd, J = 6.7, 4.5 Hz, 2H) |
| 139 | 437 | 438 | δ 10.02 (s, 1H), 9.04 (br s, 1H), 8.76 (s, 1H), 8.56 (s, 1H), 8.53 (d, J = |
| | | | 2.1 Hz, 1H), 8.34 (s, 1H), 8.33 (d, J = 2.1 Hz, 1H), 4.34-4.25 (m, |
| | | | 1H), 3.92 (t, J = 7.0 Hz, 2H), 2.56 (s, 2H), 2.12 (br t, J = 7.5 Hz, 2H), |
| | | | 2.09-2.03 (m, 2H), 1.88-1.77 (m, 4H), 1.74-1.66 (m, 1H), 1.48- |
| | | | 1.37 (m, 2H), 1.31-1.20 (m, 1H) |
| 140 | 431 | 432 | δ 10.11 (s, 1H), 9.33 (s, 1H), 9.22 (br s, 1H), 8.63 (s, 2H), 8.51 (s, |
| | | | 1H), 8.30 (s, 1H), 8.03 (br d, J = 7.9 Hz, 2H), 7.57 (br t, J = 7.8 Hz, |
| | | | 2H), 7.46-7.37 (m, 1H), 3.93 (br t, J = 6.9 Hz, 2H), 2.56 (s, 2H), |
| | | | 2.10 (br t, J = 7.4 Hz, 2H) |
| 141 | 448 | 449 | δ 10.06 (s, 1H), 8.69 (s, 1H), 8.60 (d, J = 2.0 Hz, 1H), 8.54 (s, 1H), |
| | | | 8.50 (d, J = 2.3 Hz, 1H), 7.33-7.12 (m, 2H), 6.98 (d, J = 8.3 Hz, |
| | | | 2H), 6.85-6.68 (m, 1H), 3.96-3.70 (m, 4H), 3.08 (br d, J = 12.0 Hz, |
| | | | 2H), 2.86-2.78 (m, 2H), 2.11-1.89 (m, 4H), 1.88-1.76 (m, 2H), |
| | | | 1.71 (br d, J = 12.1 Hz, 1H), 1.33-1.17 (m, 3H) |
| 142 | 457 | 458 | δ 8.74 (t, J = 2.2 Hz, 1H), 8.57 (d, J = 1.8 Hz, 1H), 8.44 (d, J = 1.8 |
| | | | Hz, 1H), 8.42 (s, 1H), 7.60 (d, J = 7.9 Hz, 1H), 7.51 (d, J = 1.8 Hz, |
| | | | 1H), 7.40 (t, J = 8.0 Hz, 1H), 7.34-7.30 (m, 2H), 7.29 (s, 1H), 7.12- |
| | | | 7.04 (m, 2H), 7.01 (d, J = 7.8 Hz, 2H), 3.84 (t, J = 7.1 Hz, 2H), 2.61- |
| | | | 2.49 (m, 2H), 2.13 (quin, J = 7.6 Hz, 2H) |
| 143 | 431 | 432 | δ10.19 (s, 1H), 8.84-8.65 (m, 4H), 8.53 (d, J = 2.3 Hz, 1H), 7.97 (d, |
| | | | J = 7.6 Hz, 2H), 7.58 (t, J = 8.0 Hz, 2H), 7.46-7.31 (m, 2H), 3.87 (t, |
| | | | J = 7.0 Hz, 2H), 2.50 (br s, 2H), 2.02 (quin, J = 7.5 Hz, 2H) |
| 144 | 423 | 424 | δ 10.07 (s, 1H), 8.78-8.68 (m, 1H), 8.68-8.63 (m, 1H), 8.62-8.58 |
| | | | (m, 1H), 8.52 (br s, 1H), 7.95 (d, J = 2.4 Hz, 1H), 7.05 (d, J = 2.3 Hz, |
| | | | 1H), 4.81 (quin, J = 6.8 Hz, 1H), 3.98-3.80 (m, 2H), 2.63-2.51 (m, |
| | | | 2H), 2.19-2.05 (m, 4H), 2.05-1.93 (m, 2H), 1.93-1.76 (m, 2H), |
| | | | 1.75 (br s, 2H) |
| 145 | 426 | 427 | δ 10.02 (s, 1H), 8.59 (d, J = 1.8 Hz, 1H), 8.57-8.53 (m, 2H), 8.50 (s, |
| | | | 1H), 3.88 (t, J = 7.0 Hz, 2H), 3.25-3.09 (m, 1H), 2.97-2.80 (m, |
| | | | 2H), 2.79-2.62 (m, 2H), 2.54 (br s, 1H), 2.11 (quin, J = 7.5 Hz, 2H), |
| | | | 2.04-1.90 (m, 3H), 1.89-1.67 (m, 5H), 1.66-1.46 (m, 4H) |
| 146 | 468 | 469 | δ 10.23 (br s, 1H), 8.87 8.79 (m, 1H), 8.66 (br d, J = 5.0 Hz, 1H), |
| | | | 8.60-8.51 (m, 2H), 4.62-4.46 (m, 1H), 4.18-4.04 (m, 1H), 3.91 (br |
| | | | t, J = 6.5 Hz, 2H), 3.71-3.64 (m, 1H), 3.59-3.47 (m, 2H), 3.37 (br |
| | | | d, J = 12.8 Hz, 1H), 3.15-3.08 (m, 1H), 3.03 (br d, J = 8.1 Hz, 1H), |
| | | | 2.12 (br t, J = 7.4 Hz, 2H), 1.96-2.03 (m, 1H), 1.89-1.77 (m, 3H), |
| | | | 1.72-1.50 (m, 8H) |
| 147 | 389 | 390 | δ 9.88 (s, 1H), 8.73 (d, J = 2.0 Hz, 1H), 8.63 (d, J = 3.5 Hz, 1H), 8.26- |
| | | | 8.15 (m, 2H), 7.80-7.71 (m, 2H), 7.45 (t, J = 7.7 Hz, 1H), 7.30 (d, |
| | | | J = 7.7 Hz, 1H), 3.98 (t, J = 7.2 Hz, 2H), 2.55 (t, J = 8.0 Hz, 2H), |
| | | | 2.09-1.98 (m, 3H), 1.09-0.95 (m, 2H), 0.78-0.67 (m, 2H) |
| 148 | 395 | 396 | δ 10.04 (s, 1H), 9.16 (s, 1H), 8.77 (s, 1H), 8.65-8.54 (m, 2H), 8.35 |
| | | | (s, 2H), 5.74 (t, J = 7.0 Hz, 1H), 5.05-4.91 (m, 4H), 3.93 (t, J = 7.0 |
| | | | Hz, 2H), 2.59 (t, J = 8.0 Hz, 2H), 2.13 (quin, J = 7.5 Hz, 2H) |
| 149 | 412 | 413 | δ 10.02 (s, 1H), 8.57 (d, J = 5102.3 Hz, 4H), 8.15 (s, 1H), 3.97-3.79 |
| | | | (m, 3H), 3.16-2.95 (m, 4H), 2.47-2.42 (m, 1H), 2.26-2.04 (m, |
| | | | 3H), 1.87-1.46 (m, 5H), 0.53-0.23 (m, 4H) |
| 150 | 447 | 448 | δ 8.83-8.81 (m, 1H), 8.32 (d, J = 6.5 Hz, 1H), 8.22 (s, 1H), 7.74- |
| | | | 7.71 (m, 2H), 7.66-7.63 (m, 1H), 7.53-7.45 (m, 3H), 3.89 (s, 1H), |
| | | | 2.13 (br d, J = 6.9 Hz, 2H), 1.88-1.81 (m, 5H), 1.72 (br d, J = 1.4 |
| | | | Hz, 1H), 1.51-1.38 (m, 6H), 1.32-1.23 (m, 2H) |
| 151 | 414 | 415 | δ 10.10 (br d, J = 5.1 Hz, 1H), 8.83-8.76 (m, 1H), 8.62-8.57 (m, |
| | | | 1H), 8.56-8.53 (m, 1H), 8.45 (dd, J = 8.3, 2.1 Hz, 1H), 4.57-4.39 |
| | | | (m, 2H), 3.92-3.87 (m, 3H), 3.20-3.09 (m, 2H), 3.03 (ddd, J = 11.2, |
| | | | 8.0, 3.6 Hz, 1H), 2.14-2.03 (m, 7H), 1.89-1.75 (m, 2H), 1.63-1.40 |
| | | | (m, 1H) |
| 152 | 414 | 415 | δ 10.12-10.02 (m, 1H), 8.72-8.62 (m, 1H), 8.60-8.44 (m, 3H), |
| | | | 4.54 (br d, J = 13.3 Hz, 1H), 4.26-4.14 (m, 1H), 3.99-3.79 (m, 3H), |
| | | | 3.70-3.57 (m, 1H), 3.24-3.08 (m, 2H), 2.71-2.59 (m, 1H), 2.15- |
| | | | 2.01 (m, 5H), 1.87-1.73 (m, 3H), 1.69-1.55 (m, 1H) |
| 153 | 468 | 469 | δ 10.52-10.36 (m, 1H), 8.81-8.71 (m, 3H), 8.61 (s, 1H), 4.63-4.55 |
| | | | (m, 1H), 4.13 (br d, J = 12.6 Hz, 1H), 3.87 (br t, J = 7.1 Hz, 3H), 3.34 |
| | | | (ddd, J = 15.4, 7.8, 3.6 Hz, 2H), 3.24-3.13 (m, 2H), 3.01 (td, J = |
| | | | 15.6, 8.0 Hz, 1H), 2.76-2.63 (m, 2H), 2.61-2.53 (m, 3H), 2.13 |
| | | | (quin, J = 7.5 Hz, 2H), 1.73-1.56 (m, 7H) |
| 154 | 396 | 397 | δ 9.91 (s, 1H), 8.63-8.59 (m, 2H), 8.53 (d, J = 2.1 Hz, 1H), 8.49 (d, |
| | | | J = 2.0 Hz, 1H), 8.15 (s, 1H), 3.90 (t, J = 7.1 Hz, 2H), 3.10-2.96 (m, |
| | | | 4H), 2.27-2.04 (m, 4H), 1.89-1.81 (m, 1H), 1.77-1.47 (m, 5H), |
| | | | 0.48-0.30 (m, 4H) |
| 155 | 422 | 423 | δ 9.03 (t, J = 2.2 Hz, 1H), 8.57 (d, J = 2.1 Hz, 1H), 8.46 (d, J = 2.0 |
| | | | Hz, 1H), 8.29 (d, J = 3.8 Hz, 1H), 8.21 (d, J = 2.5 Hz, 1H), 8.19 (d, |
| | | | J = 2.0 Hz, 1H), 8.14 (d, J = 1.9 Hz, 1H), 6.60 (d, J = 9.8 Hz, 1H), 5.72 |
| | | | (s, 1H), 5.08-5.04 (m, 2H), 4.80 (s, 2H), 3.88 (t, J = 7.1 Hz, 2H), |
| | | | 2.58 (t, J = 8.1 Hz, 2H), 2.16 (s, 2H) |
| 156 | 465 | 466 | δ 9.71 (s, 1H), 8.56-8.47 (m, 2H), 8.40 (d, J = 2.0 Hz, 1H), 8.17 (s, |
| | | | 1H), 7.82-7.74 (m, 1H), 7.41 (ddd, J = 1.9, 6.8, 8.9 Hz, 1H), 6.40 (d, |
| | | | J = 9.1 Hz, 1H), 6.31-6.25 (m, 1H), 4.89-4.77 (m, 1H), 4.33 (br d, |
| | | | J = 12.4 Hz, 1H), 4.24 (br d, J = 9.0 Hz, 1H), 3.78-3.72 (m, 2H), |
| | | | 3.24 (br t, J = 11.6 Hz, 1H), 2.95 (br t, J = 11.9 Hz, 1H), 2.46-2.39 |
| | | | (m, 2H), 2.08-1.96 (m, 3H), 1.89 (br s, 2H), 1.83-1.68 (m, 1H) |
| 157 | 420 | 421 | δ 9.96 (s, 1H), 8.70 (s, 1H), 8.65 (d, J = 2.0 Hz, 1H), 8.60 (d, J = 3.7 |
| | | | Hz, 1H), 8.51-8.36 (m, 2H), 8.15 (dd, J = 2.1, 9.5 Hz, 1H), 6.53 (d, |
| | | | J = 9.7 Hz, 1H), 5.06-4.86 (m, 1H), 3.87 (t, J = 7.0 Hz, 2H), 2.61- |
| | | | 2.51 (m, 2H), 2.45-2.35 (m, 2H), 2.27 (br t, J = 10.4 Hz, 2H), 2.10 |
| | | | (br t, J = 7.5 Hz, 2H), 1.88-1.74 (m, 2H) |
| 158 | 420 | 421 | δ 9.99 (br s, 1H), 8.87 (br s, 1H), 8.71-8.52 (m, 3H), 8.36 (d, J = 1.8 |
| | | | Hz, 1H), 8.25-8.01 (m, 1H), 6.58 (d, J = 9.5 Hz, 1H), 3.92 (br d, J = |
| | | | 7.3 Hz, 2H), 3.88 (br t, J = 7.2 Hz, 2H), 2.59-2.53 (m, 2H), 2.10 (br |
| | | | t, J = 7.3 Hz, 2H), 1.33-1.18 (m, 1H), 0.58-0.37 (m, 4H) |
| 159 | 480 | 481 | δ 10.08 (br d, J = 19.89 Hz, 1H), 8.58-8.68 (m, 2H), 8.50-8.58 (m, |
| | | | 1H), 8.48 (br s, 1H), 7.55-7.63 (m, 2H), 7.22-7.30 (m, 2H), 3.68- |
| | | | 3.94 (m, 6H), 3.60 (br t, J = 6.88 Hz, 1H), 2.45-2.49 (m, 1H), 2.20- |
| | | | 2.41 (m, 2H), 2.01-2.19 (m, 3H) |
| 160 | 456 | 457 | δ 10.11 (s, 1H), 8.87-8.76 (m, 1H), 8.62-8.52 (m, 2H), 8.49-8.39 |
| | | | (m, 1H), 4.78-4.70 (m, 3H), 4.69-4.60 (m, 2H), 4.60-4.45 (m, |
| | | | 1H), 4.25-4.07 (m, 1H), 3.90 (t, J = 7.0 Hz, 2H), 3.55-3.36 (m, |
| | | | 2H), 3.16-2.89 (m, 2H), 2.74-2.63 (m, 1H), 2.18-2.04 (m, 2H), |
| | | | 2.02-1.71 (m, 3H), 1.58-1.40 (m, 1H) |
| 161 | 493 | 494 | δ 12.10-11.29 (m, 1H), 10.19-9.88 (m, 1H), 8.83-8.71 (m, 1H), |
| | | | 8.66-8.50 (m, 2H), 8.48-8.40 (m, 1H), 7.72-7.41 (m, 1H), 6.72- |
| | | | 6.19 (m, 2H), 4.63-4.41 (m, 1H), 3.89 (br t, J = 6.4 Hz, 3H), 3.75- |
| | | | 3.57 (m, 1H), 3.26-3.07 (m, 2H), 2.96-2.76 (m, 1H), 2.20-2.08 |
| | | | (m, 2H), 2.00 (br s, 2H), 1.91-1.40 (m, 3H) |
| 162 | 494 | 495 | δ 10.37-10.20 (m, 1H), 8.84 (br s, 1H), 8.70 (br s, 1H), 8.63-8.44 |
| | | | (m, 2H), 7.50 (br s, 2H), 7.28 (br t, J = 8.3 Hz, 2H), 4.70-4.46 (m, |
| | | | 1H), 3.90 (br t, J = 6.9 Hz, 2H), 3.77-3.57 (m, 1H), 3.23 (br s, 2H), |
| | | | 2.59-2.53 (m, 2H), 2.16-2.09 (m, 2H), 2.01 (br s, 2H), 1.92-1.71 |
| | | | (m, 2H), 1.68-1.54 (m, 1H) |
| 163 | 493 | 494 | δ 11.95-11.80 (m, 1H), 10.11 (s, 1H), 8.78 (br s, 1H), 8.59 (d, J = |
| | | | 2.1 Hz, 1H), 8.54 (s, 1H), 8.46 (d, J = 2.2 Hz, 1H), 7.63 (s, 1H), 7.53 |
| | | | (dd, J = 2.6, 9.4 Hz, 1H), 6.36 (d, J = 9.4 Hz, 1H), 3.90 (t, J = 7.0 Hz, |
| | | | 3H), 3.26-3.18 (m, 2H), 3.13-2.95 (m, 1H), 2.49-2.44 (m, 1H), |
| | | | 2.12 (quin, J = 7.5 Hz, 3H), 2.05-1.88 (m, 3H), 1.85-1.80 (m, 1H), |
| | | | 1.67-1.58 (m, 1H) |
| 164 | 493 | 494 | δ 12.07-11.35 (m, 1H), 10.49-10.15 (m, 1H), 8.84 (br d, J = 11.8 |
| | | | Hz, 1H), 8.69 (br d, J = 18.4 Hz, 1H), 8.63-8.41 (m, 2H), 7.45 (br |
| | | | dd, J = 6.5, 14.0 Hz, 1H), 6.29 (br d, J = 6.4 Hz, 1H), 6.15 (br dd, J = |
| | | | 6.3, 19.3 Hz, 1H), 4.63-4.46 (m, 1H), 4.00-3.82 (m, 2H), 3.79- |
| | | | 3.55 (m, 1H), 3.44-3.04 (m, 3H), 2.96-2.65 (m, 1H), 2.58 (s, 1H), |
| | | | 2.13 (quin, J = 7.3 Hz, 2H), 2.06-1.71 (m, 3H), 1.71-1.48 (m, 1H) |
| 165 | 493 | 494 | δ 11.89 (br s, 1H), 10.11-9.98 (m, 1H), 8.79-8.68 (m, 1H), 8.62- |
| | | | 8.42 (m, 3H), 7.55-7.38 (m, 2H), 6.23 (td, J = 6.5, 12.9 Hz, 1H), |
| | | | 4.65-4.48 (m, 1H), 3.88 (q, J = 6.8 Hz, 2H), 3.62-3.44 (m, 1H), |
| | | | 3.18-3.00 (m, 2H), 2.79 (br s, 1H), 2.57-2.52 (m, 2H), 2.11 (quin, |
| | | | J = 7.5 Hz, 2H), 2.03-1.93 (m, 1H), 1.85 (br d, J = 12.0 Hz, 1H), 1.76- |
| | | | 1.50 (m, 2H) |

TABLE 4
Spectrometric data for compounds of Table 2

| Cmpd No | MS calc | MS found | ¹H NMR Data (400 MHz, DMSO-d₆) |
|---|---|---|---|
| 51 | 857 | 858 | δ 11.08 (s, 1H), 10.29 (br s, 1H), 10.19 (br s, 1H), 8.72 (s, 2H), 8.58 |
| | | | (s, 1H), 8.46 (s, 1H), 8.14-8.09 (m, 1H), 7.99 (t, J = 6.0 Hz, 1H), |
| | | | 7.93-7.81 (m, 2H), 7.77-7.69 (m, 2H), 7.68-7.62 (m, 1H), 7.55- |
| | | | 7.48 (m, 2H), 7.45-7.39 (m, 1H), 7.08 (d, J = 8.5 Hz, 1H), 6.86 (d, |
| | | | J = 1.6 Hz, 1H), 6.76-6.70 (m, 1H), 5.40-5.27 (m, 1H), 4.42 (d, J = |
| | | | 3.8 Hz, 2H), 3.17-3.08 (m, 3H), 2.95-2.84 (m, 1H), 2.76-2.59 (m, |
| | | | 2H), 2.37-2.27 (m, 3H), 2.05-1.93 (m, 1H), 1.62-1.53 (m, 2H), |
| | | | 1.46-1.37 (m, 2H), 1.31-1.18 (m, 11H), 0.86 (t, J = 7.3 Hz, 3H) |
| 52 | 908 | 909 | δ 10.96 (br s, 1H), 10.23 (s, 1H), 8.76-8.66 (m, 2H), 8.61 (dd, J = |
| | | | 16.2, 1.9 Hz, 2H), 8.15 (s, 1H), 7.92-7.82 (m, 2H), 7.79-7.69 (m, |
| | | | 2H), 7.69-7.56 (m, 2H), 7.55-7.45 (m, 2H), 7.45-7.37 (m, 1H), |
| | | | 7.14 (s, 1H), 7.03 (dd, J = 8.4, 1.9 Hz, 1H), 5.14-4.96 (m, 1H), 4.47- |
| | | | 4.19 (m, 2H), 4.05 (br t, J = 6.4 Hz, 2H), 2.98-2.82 (m, 2H), 2.74 |
| | | | (br d, J = 11.4 Hz, 2H), 2.60 (br d, J = 2.3 Hz, 2H), 2.40-2.31 (m, |
| | | | 1H), 2.29-2.18 (m, 2H), 2.02-1.83 (m, 3H), 1.82-1.62 (m, 6H), |
| | | | 1.32-1.31 (m, 1H), 1.47-1.17 (m, 15H) |
| 53 | 894 | 895 | δ 10.96 (s, 1H), 10.23 (s, 1H), 8.74-8.68 (m, 2H), 8.62 (dd, J = |
| | | | 15.0, 2.1 Hz, 2H), 8.15 (s, 1H), 7.88 (br t, J = 9.4 Hz, 2H), 7.76 (d, |
| | | | J = 7.3 Hz, 2H), 7.67-7.59 (m, 2H), 7.54-7.48 (m, 2H), 7.45-7.36 |
| | | | (m, 1H), 7.15 (d, J = 1.7 Hz, 1H), 7.03 (dd, J = 8.4, 2.1 Hz, 1H), 5.06 |
| | | | (dd, J = 13.3, 5.1 Hz, 1H), 4.44-4.33 (m, 1H), 4.29-4.21 (m, 1H), |
| | | | 4.11-4.01 (m, 2H), 3.64 (br t, J = 6.7 Hz, 2H), 2.96-2.83 (m, 1H), |
| | | | 2.75 (br d, J = 11.5 Hz, 2H), 2.58 (br dd, J = 15.3, 1.8 Hz, 1H), 2.44- |
| | | | 2.31 (m, 1H), 2.26 (br t, J = 7.3 Hz, 2H), 2.03-1.86 (m, 3H), 1.82- |
| | | | 1.60 (m, 6H), 1.48-1.18 (m, 14H) |
| 54 | 866 | 867 | δ 10.96 (s, 1H), 10.23 (s, 1H), 8.72 (s, 1H), 8.70 (s, 1H), 8.62 (dd, |
| | | | J = 12.4, 1.9 Hz, 2H), 8.17 (d, J = 9.1 Hz, 2H), 7.88 (br t, J = 9.4 Hz, |
| | | | 2H), 7.76 (d, J = 7.4 Hz, 2H), 7.69-7.59 (m, 2H), 7.55-7.47 (m, |
| | | | 2H), 7.45-7.39 (m, 1H), 7.16 (d, J = 1.6 Hz, 1H), 7.04 (dd, J = 8.4, |
| | | | 2.1 Hz, 1H), 5.06 (dd, J = 13.3, 5.1 Hz, 1H), 4.40-4.22 (m, 2H), |
| | | | 4.06 (br t, J = 6.4 Hz, 2H), 3.67-3.64 (m, 2H), 2.93-2.87 (m, 1H), |
| | | | 2.79 (br d, J = 9.1 Hz, 2H), 2.70-2.51 (m, 2H), 2.40-2.30 (m, 3H), |
| | | | 2.02-1.94 (m, 3H), 1.82-1.68 (m, 6H), 1.48-1.41 (m, 4H), 1.37- |
| | | | 1.26 (m, 5H) |
| 55 | 802 | 803 | δ 11.15-11.03 (m, 1H), 10.24 (s, 1H), 8.73 (s, 2H), 8.62 (dd, J = |
| | | | 5.8, 1.9 Hz, 2H), 8.16 (s, 1H), 7.88 (br dd, J = 11.6, 8.1 Hz, 2H), |
| | | | 7.83-7.73 (m, 3H), 7.65 (t, J = 7.7 Hz, 1H), 7.55-7.48 (m, 3H), |
| | | | 7.46-7.39 (m, 2H), 5.12-5.05 (m, 1H), 4.20 (br t, J = 6.3 Hz, 3H), |
| | | | 3.85-3.77 (m, 1H), 3.71 (br t, J = 6.6 Hz, 2H), 3.18-3.09 (m, 1H), |
| | | | 2.95-2.73 (m, 2H), 2.35-2.27 (m, 3H), 2.08-1.99 (m, 1H), 1.93 |
| | | | (br t, J = 6.1 Hz, 2H), 1.80-1.71 (m, 2H), 1.67-1.58 (m, 1H), 1.55- |
| | | | 1.42 (m, 7H), 1.38-1.26 (m, 9H) |
| 56 | 880 | 881 | δ 10.96 (s, 1H), 10.23 (s, 1H), 8.73 (s, 1H), 8.69 (s, 1H), 8.62 (dd, |
| | | | J = 13.6, 1.9 Hz, 2H), 8.21-8.14 (m, 2H), 7.88 (t, J = 9.3 Hz, 2H), |
| | | | 7.76 (d, J = 7.4 Hz, 2H), 7.68-7.59 (m, 2H), 7.51 (t, J = 7.6 Hz, |
| | | | 2H), 7.45-7.38 (m, 1H), 7.16 (s, 1H), 7.04 (dd, J = 8.4, 2.0 Hz, 1H), |
| | | | 5.06 (dd, J = 13.3, 5.1 Hz, 1H), 4.42-4.20 (m, 2H), 4.06 (t, J = 6.4 |
| | | | Hz, 2H), 3.65 (br t, J = 6.7 Hz, 2H), 2.95-2.85 (m, 1H), 2.81-2.75 |
| | | | (m, 2H), 2.67-2.53 (m, 2H), 2.43-2.28 (m, 3H), 2.01-1.90 (m, |
| | | | 3H), 1.82-1.65 (m, 6H), 1.42 (br d, J = 13.6 Hz, 5H), 1.36-1.27 |
| | | | (m, 6H) |
| 57 | 856 | 857 | δ 11.19-11.06 (m, 1H), 10.53-10.41 (m, 1H), 9.06-8.98 (m, 1H), |
| | | | 8.83-8.72 (m, 1H), 8.64-8.50 (m, 1H), 8.38-8.30 (m, 1H), 8.15- |
| | | | 8.07 (m, 1H), 7.97 (br d, J = 8.6 Hz, 1H), 7.93-7.78 (m, 4H), 7.74 |
| | | | (br d, J = 7.5 Hz, 2H), 7.70-7.64 (m, 1H), 7.55-7.47 (m, 3H), 7.45- |
| | | | 7.36 (m, 2H), 5.16-5.07 (m, 1H), 4.81-4.71 (m, 2H), 3.26 (br |
| | | | d, J = 6.1 Hz, 2H), 3.13 (br d, J = 5.9 Hz, 2H), 2.92-2.82 (m, 2H), |
| | | | 2.57 (br s, 2H), 2.13-1.95 (m, 2H), 1.50 (br s, 2H), 1.42 (br s, 2H), |
| | | | 1.24 (br s, 8H) |
| 58 | 883 | 884 | δ 10.14 (s, 1H), 10.05 (s, 1H), 8.69 (s, 1H), 8.61 (d, J = 2.2 Hz, 1H), |
| | | | 8.56 (s, 1H), 8.35 (d, J = 1.5 Hz, 1H), 8.16 (s, 1H), 8.11 (s, 1H), 7.90- |
| | | | 7.78 (m, 4H), 7.75-7.71 (m, 2H), 7.67-7.62 (m, 1H), 7.51 (d, J = |
| | | | 8.2 Hz, 3H), 7.46-7.40 (m, 2H), 5.11-5.05 (m, 1H), 4.30 (q, J = |
| | | | 5.7 Hz, 2H), 4.26-4.20 (m, 2H), 2.68-2.55 (m, 5H), 2.38-2.20 (m, |
| | | | 11H), 1.92-1.85 (m, 2H), 1.60-1.53 (m, 2H), 1.43-1.36 (m, 2H), |
| | | | 1.29 (br s, 4H) |
| 59 | 856 | 857 | δ 11.12 (br s, 1H), 10.14 (s, 1H), 10.06-10.03 (m, 1H), 8.70 (s, 1H), |
| | | | 8.63 (d, J = 2.3 Hz, 1H), 8.55 (s, 1H), 8.36 (d, J = 1.9 Hz, 1H), 8.18 |
| | | | (t, J = 5.6 Hz, 1H), 8.13-8.10 (m, 1H), 7.91-7.83 (m, 3H), 7.73 (d, |
| | | | J = 7.2 Hz, 2H), 7.68-7.62 (m, 1H), 7.54-7.47 (m, 2H), 7.45-7.36 |
| | | | (m, 3H), 5.12 (m, 1H), 4.71 (s, 2H), 3.12 (m, 2H), 2.94-2.83 (m, |
| | | | 1H), 2.61 (br d, J = 2.8 Hz, 1H), 2.59-2.53 (m, 1H), 2.30 (t, J = 7.4 |
| | | | Hz, 2H), 2.09-2.00 (m, 1H), 1.61-1.52 (m, 2H), 1.47-1.38 (m, |
| | | | 2H), 1.28-1.22 (m, 10H) |
| 60 | 842 | 843 | δ 10.97 (s, 1H), 10.54-10.21 (m, 2H), 8.75 (br d, J = 1.3 Hz, 2H), |
| | | | 8.63-8.58 (m, 1H), 8.56-8.41 (m, 1H), 8.13-8.09 (m, 2H), 7.87 (t, |
| | | | J = 6.9 Hz, 2H), 7.74 (d, J = 7.3 Hz, 2H), 7.70-7.63 (m, 2H), 7.54- |
| | | | 7.48 (m, 2H), 7.44-7.39 (m, 1H), 7.16 (d, J = 1.9 Hz, 1H), 7.09 (dd, |
| | | | J = 8.5, 2.1 Hz, 1H), 5.12-5.04 (m, 1H), 4.57 (s, 2H), 4.44-4.34 |
| | | | (m, 1H), 4.30-4.22 (m, 1H), 3.13-3.09 (m, 2H), 2.96-2.85 (m, |
| | | | 1H), 2.69-2.55 (m, 1H), 2.35-2.32 (m, 2H), 2.03-1.93 (m, 1H), |
| | | | 1.60-1.54 (m, 2H), 1.46-1.38 (m, 2H), 1.29-1.22 (m, 11H) |
| 61 | 922 | 923 | δ 10.96 (s, 1H), 10.24 (s, 1H), 8.76-8.69 (m, 2H), 8.61 (dd, J = 4.7, |
| | | | 2.1 Hz, 2H), 8.15 (s, 1H), 7.88 (dd, J = 12.5, 8.1 Hz, 2H), 7.75 (d, |
| | | | J = 7.4 Hz, 2H), 7.68-7.58 (m, 2H), 7.55-7.47 (m, 2H), 7.45-7.38 |
| | | | (m, 1H), 7.14 (d, J = 1.7 Hz, 1H), 7.03 (dd, J = 8.3, 1.9 Hz, 1H), 5.06 |
| | | | (dd, J = 13.2, 5.0 Hz, 1H), 4.42-4.16 (m, 3H), 4.04 (t, J = 6.4 Hz, |
| | | | 2H), 3.86-3.75 (m, 1H), 3.71 (br t, J = 6.8 Hz, 2H), 3.16-3.08 (m, |
| | | | 1H), 2.97-2.83 (m, 1H), 2.83-2.72 (m, 1H), 2.65-2.54 (m, 1H), |
| | | | 2.39-2.28 (m, 3H), 2.01-1.89 (m, 3H), 1.81-1.69 (m, 2H), 1.68- |
| | | | 1.59 (m, 1H), 1.53-1.39 (m, 7H), 1.37-1.23 (m, 9H) |
| 62 | 908 | 909 | δ 10.96 (s, 1H), 10.36 (br s, 1H), 8.78 (br s, 1H), 8.75 (s, 1H), 8.67 |
| | | | (br s, 2H), 8.15 (s, 1H), 7.88 (t, J = 8.8 Hz, 2H), 7.76 (d, J = 7.5 Hz, |
| | | | 2H), 7.69-7.58 (m, 2H), 7.55-7.47 (m, 2H), 7.45-7.38 (m, 1H), |
| | | | 7.15 (s, 1H), 7.03 (dd, J = 8.5, 1.6 Hz, 1H), 5.06 (dd, J = 13.1, 4.8 |
| | | | Hz, 1H), 4.41-4.17 (m, 3H), 4.05 (br t, J = 6.4 Hz, 2H), 3.83-3.77 |
| | | | (m, 1H), 3.72 (br t, J = 6.6 Hz, 2H), 3.13 (br t, J = 11.9 Hz, 1H), 2.96- |
| | | | 2.85 (m, 1H), 2.83-2.75 (m, 1H), 2.58 (br d, J = 17.4 Hz, 1H), |
| | | | 2.44-2.27 (m, 3H), 2.05-1.87 (m, 3H), 1.83-1.69 (m, 2H), 1.68- |
| | | | 1.58 (m, 1H), 1.55-1.38 (m, 7H), 1.37-1.21 (m, 6H) |
| 63 | 894 | 895 | δ 10.96 (s, 1H), 10.43 (br d, J = 1.1 Hz, 1H), 8.85-8.78 (m, 1H), |
| | | | 8.76 (s, 1H), 8.71 (br s, 2H), 8.15 (s, 1H), 7.88 (t, J = 8.0 Hz, 2H), |
| | | | 7.79-7.72 (m, 2H), 7.69-7.58 (m, 2H), 7.55-7.47 (m, 2H), 7.45- |
| | | | 7.37 (m, 1H), 7.15 (s, 1H), 7.03 (dd, J = 8.4, 2.1 Hz, 1H), 5.06 (dd, |
| | | | J = 13.2, 5.1 Hz, 1H), 4.41-4.18 (m, 3H), 4.06 (t, J = 6.4 Hz, 2H), |
| | | | 3.84-3.78 (m, 1H), 3.73 (br t, J = 6.7 Hz, 2H), 3.17-3.09 (m, 1H), |
| | | | 2.94-2.85 (m, 1H), 2.83-2.74 (m, 1H), 2.58 (br d, J = 17.2 Hz, |
| | | | 1H), 2.45-2.29 (m, 3H), 2.03-1.89 (m, 3H), 1.81-1.68 (m, 2H), |
| | | | 1.68-1.59 (m, 1H), 1.55-1.39 (m, 7H), 1.38-1.26 (m, 4H) |
| 64 | 880 | 881 | δ 10.95 (s, 1H), 10.24 (s, 1H), 8.72 (s, 2H), 8.61 (dd, J = 5.1, 2.1 Hz, |
| | | | 2H), 8.15 (s, 1H), 7.94-7.82 (m, 2H), 7.75 (d, J = 7.4 Hz, 2H), 7.68- |
| | | | 7.59 (m, 2H), 7.55-7.47 (m, 2H), 7.45-7.39 (m, 1H), 7.16 (s, |
| | | | 1H), 7.04 (dd, J = 8.4, 2.0 Hz, 1H), 5.06 (dd, J = 13.2, 5.1 Hz, 1H), |
| | | | 4.44-4.16 (m, 3H), 4.06 (t, J = 6.4 Hz, 2H), 3.87-3.76 (m, 1H), |
| | | | 3.71 (br t, J = 6.7 Hz, 2H), 3.13 (br t, J = 11.9 Hz, 2H), 2.97-2.84 |
| | | | (m, 1H), 2.78 (br t, J = 11.2 Hz, 1H), 2.69-2.53 (m, 2H), 2.43-2.26 |
| | | | (m, 4H), 2.07-1.87 (m, 3H), 1.75 (quin, J = 6.8 Hz, 2H), 1.68-1.30 |
| | | | (m, 11H) |
| 65 | 855 | 856 | δ = 10.14 (s, 1H), 10.05 (s, 1H), 8.69 (s, 1H), 8.62 (d, J = 2.1 Hz, |
| | | | 1H), 8.55 (s, 1H), 8.35 (s, 1H), 8.15 (s, 1H), 8.11 (s, 1H), 7.90-7.78 |
| | | | (m, 4H), 7.73 (br d, J = 7.2 Hz, 2H), 7.67-7.63 (m, 1H), 7.52-7.48 |
| | | | (m, 3H), 7.43 (dd, J = 10.2, 7.4 Hz, 2H), 4.23 (br t, J = 5.9 Hz, 3H), |
| | | | 2.94-2.81 (m, 2H), 2.73-2.66 (m, 1H), 2.60 (br d, J = 2.3 Hz, 1H), |
| | | | 2.56 (br s, 2H), 2.34-2.25 (m, 7H), 2.06-1.99 (m, 1H), 1.90 (quin, |
| | | | J = 6.4 Hz, 3H), 1.60-1.54 (m, 3H), 1.49-1.40 (m, 3H) |
| 66 | 842 | 843 | δ 11.03-10.99 (m, 1H), 10.16-10.12 (m, 1H), 10.05 (s, 1H), 8.70 |
| | | | (s, 1H), 8.63 (d, J = 2.2 Hz, 1H), 8.55 (s, 1H), 8.36 (d, J = 1.7 Hz, |
| | | | 1H), 8.13-8.03 (m, 2H), 7.87 (m, 2H), 7.76-7.71 (m, 2H), 7.68- |
| | | | 7.62 (m, 1H), 7.53-7.39 (m, 4H), 7.35 (d, J = 7.5 Hz, 1H), 7.12 (d, |
| | | | J = 8.1 Hz, 1H), 5.14 (m, 1H), 4.62 (s, 2H), 4.49-4.42 (m, 1H), 4.39- |
| | | | 4.31 (m, 1H), 3.11 (m, 2H), 2.99-2.87 (m, 1H), 2.65-2.57 (m, 1H), |
| | | | 2.42 (m, 1H), 2.34-2.26 (m, 2H), 2.05-1.97 (m, 1H), 1.52-1.51 |
| | | | (m, 2H), 1.45-1.36 (m, 2H), 1.29-1.21 (m, 10H) |
| 67 | 897 | 898 | δ = 8.73 (s, 1H), 8.66 (d, J = 2.1 Hz, 1H), 8.62 (br s, 1H), 8.55 (s, |
| | | | 1H), 8.50 (s, 1H), 8.39 (s, 1H), 8.12 (s, 1H), 7.91 (d, J = 7.9 Hz, 1H), |
| | | | 7.73 (d, J = 7.6 Hz, 1H), 7.69 (s, 1H), 7.68-7.64 (m, 2H), 7.61- |
| | | | 7.56 (m, 1H), 7.49-7.46 (m, 2H), 7.40-7.35 (m, 1H), 7.21 (d, J = |
| | | | 8.6 Hz, 1H), 4.94 (dd, J = 12.2, 5.5 Hz, 1H), 4.29-4.14 (m, 2H), |
| | | | 2.90-2.79 (m, 4H), 2.77-2.65 (m, 8H), 2.35 (br t, J = 7.3 Hz, 3H), |
| | | | 2.31-2.19 (m, 5H), 2.15-2.08 (m, 2H), 2.08-2.02 (m, 2H), 1.77- |
| | | | 1.58 (m, 4H), 1.35 (br s, 4H) |
| 68 | 869 | 870 | δ = 11.10 (s, 1H), 10.14 (s, 1H), 10.05 (s, 1H), 8.69 (s, 1H), 8.62 (d, |
| | | | J = 2.1 Hz, 1H), 8.55 (s, 1H), 8.35 (d, J = 1.8 Hz, 1H), 8.15-8.10 |
| | | | (m, 1H), 7.91-7.77 (m, 3H), 7.75-7.70 (m, 2H), 7.67-7.61 (m, |
| | | | 1H), 7.53-7.47 (m, 3H), 7.46-7.38 (m, 2H), 5.08 (dd, J = 12.8, 5.3 |
| | | | Hz, 1H), 4.23 (br t, J = 6.2 Hz, 2H), 2.88 (ddd, J = 17.2, 13.8, 5.1 |
| | | | Hz, 1H), 2.62-2.52 (m, 3H), 2.46 (br t, J = 7.0 Hz, 3H), 2.39 (br d, |
| | | | J = 1.8 Hz, 4H), 2.33-2.25 (m, 5H), 2.15-1.98 (m, 2H), 1.94-1.85 |
| | | | (m, 2H), 1.58 (quin, J = 7.2 Hz, 2H), 1.48-1.39 (m, 2H), 1.34-1.24 |
| | | | (m, 2H) |
| 69 | 922 | 923 | δ 10.99-10.95 (m, 1H), 10.25 (s, 1H), 8.73 (s, 2H), 8.62 (dd, J = |
| | | | 5.0, 2.2 Hz, 2H), 8.18 (s, 1H), 7.92-7.84 (m, 2H), 7.76 (d, J = 7.6 |
| | | | Hz, 2H), 7.67-7.62 (m, 1H), 7.54-7.49 (m, 2H), 7.48-7.40 (m, |
| | | | 2H), 7.30 (d, J = 7.4 Hz, 1H), 7.23 (d, J = 8.3 Hz, 1H), 5.15-5.07 |
| | | | (m, 1H), 4.40-4.34 (m, 1H), 4.27-4.18 (m, 2H), 4.14-4.08 (m, |
| | | | 2H), 3.84-3.77 (m, 1H), 3.76-3.68 (m, 2H), 3.17-3.07 (m, 1H), |
| | | | 2.97 (s, 1H), 2.83-2.72 (m, 1H), 2.35-2.29 (m, 2H), 2.03-1.89 |
| | | | (m, 3H), 1.74 (quin, J = 6.9 Hz, 2H), 1.68-1.60 (m, 1H), 1.54-1.40 |
| | | | (m, 8H), 1.36-1.26 (m, 9H) |
| 70 | 894 | 895 | δ 10.97 (s, 1H), 10.32 (s, 1H), 8.78-8.72 (m, 2H), 8.66 (br d, J = 2.1 |
| | | | Hz, 2H), 8.30-8.04 (m, 1H), 7.89 (t, J = 9.2 Hz, 2H), 7.76 (d, J = |
| | | | 7.6 Hz, 2H), 7.69-7.62 (m, 1H), 7.57-7.37 (m, 4H), 7.30 (d, J = |
| | | | 7.5 Hz, 1H), 7.24 (d, J = 8.2 Hz, 1H), 5.11 (dd, J = 13.3, 5.0 Hz, |
| | | | 1H), 4.42-4.32 (m, 1H), 4.29-4.16 (m, 2H), 4.12 (t, J = 6.3 Hz, |
| | | | 2H), 3.81 (br d, J = 13.1 Hz, 1H), 3.72 (br t, J = 6.7 Hz, 2H), 3.13 (br |
| | | | t, J = 11.7 Hz, 1H), 2.97-2.84 (m, 1H), 2.79 (br t, J = 11.2 Hz, 1H), |
| | | | 2.66-2.54 (m, 1H), 2.47-2.37 (m, 1H), 2.36-2.27 (m, 2H), 2.04- |
| | | | 1.87 (m, 3H), 1.82-1.69 (m, 2H), 1.68-1.59 (m, 1H), 1.56-1.42 |
| | | | (m, 7H), 1.35 (br d, J = 2.3 Hz, 4H) |
| 71 | 908 | 909 | δ 10.97 (s, 1H), 10.25 (s, 1H), 8.72 (s, 2H), 8.62 (dd, J = 4.7, 2.1 Hz, |
| | | | 2H), 8.16 (s, 1H), 7.88 (br dd, J = 12.3, 7.9 Hz, 2H), 7.76 (d, J = 7.4 |
| | | | Hz, 2H), 7.68-7.62 (m, 1H), 7.54-7.48 (m, 2H), 7.47-7.39 (m, |
| | | | 2H), 7.30 (d, J = 7.5 Hz, 1H), 7.23 (d, J = 8.1 Hz, 1H), 5.11 (dd, J = |
| | | | 13.3, 5.1 Hz, 1H), 4.41-4.32 (m, 1H), 4.27-4.19 (m, 2H), 4.11 (br |
| | | | t, J = 6.4 Hz, 2H), 3.80 (br d, J = 13.4 Hz, 1H), 3.71 (br t, J = 6.8 Hz, |
| | | | 2H), 3.12 (br t, J = 11.6 Hz, 2H), 2.98-2.84 (m, 1H), 2.78 (br t, J = |
| | | | 10.8 Hz, 1H), 2.58 (br d, J = 17.4 Hz, 1H), 2.46 (br dd, J = 13.1, 4.4 |
| | | | Hz, 1H), 2.32 (q, J = 7.1 Hz, 2H), 2.03-1.96 (m, 1H), 1.92 (br t, J = |
| | | | 6.3 Hz, 2H), 1.78-1.70 (m, 2H), 1.66-1.58 (m, 1H), 1.52-1.42 (m, |
| | | | 6H), 1.32 (br s, 6H) |
| 72 | 922 | 923 | δ 11.11 (s, 1H), 10.24 (s, 1H), 8.73 (s, 2H), 8.64-8.57 (m, 2H), 8.16 |
| | | | (s, 1H), 7.88 (br dd, J = 11.6, 7.8 Hz, 2H), 7.82 (d, J = 8.4 Hz, 1H), |
| | | | 7.76 (br d, J = 7.8 Hz, 2H), 7.65 (t, J = 7.8 Hz, 1H), 7.55-7.49 (m, |
| | | | 2H), 7.45-7.39 (m, 2H), 7.34 (dd, J = 8.4, 2.1 Hz, 1H), 5.12 (dd, J = |
| | | | 12.6, 5.4 Hz, 1H), 4.25-4.14 (m, 3H), 3.84-3.78 (m, 1H), 3.71 (br |
| | | | d, J = 7.1 Hz, 2H), 3.14 (br s, 2H), 2.94-2.74 (m, 4H), 2.57 (br s, |
| | | | 1H), 2.33 (br d, J = 6.3 Hz, 2H), 2.08-2.02 (m, 1H), 1.92 (br d, J = |
| | | | 5.5 Hz, 2H), 1.78-1.73 (m, 2H), 1.62 (br d, J = 10.8 Hz, 1H), 1.53- |
| | | | 1.47 (m, 3H), 1.44 (br s, 2H), 1.33 (br s, 6H) |
| 73 | 908 | 909 | δ 11.11 (s, 1H), 10.25 (s, 1H), 8.73 (s, 2H), 8.62 (dd, J = 6.3, 2.1 Hz, |
| | | | 2H), 8.16 (s, 1H), 7.92-7.85 (m, 2H), 7.82 (d, J = 8.3 Hz, 1H), 7.76 |
| | | | (d, J = 7.2 Hz, 2H), 7.65 (t, J = 7.8 Hz, 1H), 7.55-7.48 (m, 2H), |
| | | | 7.45-7.39 (m, 2H), 7.35 (dd, J = 8.4, 2.3 Hz, 1H), 5.12 (dd, J = |
| | | | 13.0, 5.4 Hz, 1H), 4.22-4.17 (m, 2H), 3.85-3.67 (m, 3H), 3.19- |
| | | | 3.07 (m, 1H), 2.94-2.75 (m, 2H), 2.63-2.53 (m, 2H), 2.38-2.29 |
| | | | (m, 2H), 2.09-2.00 (m, 1H), 1.93 (br t, J = 6.4 Hz, 2H), 1.80-1.73 |
| | | | (m, 2H), 1.58-1.26 (m, 13H) |
| 74 | 894 | 895 | δ 11.11 (br s, 1H), 10.25 (s, 1H), 8.75-8.72 (m, 2H), 8.62 (dd, J = |
| | | | 6.6, 2.0 Hz, 2H), 8.16 (s, 1H), 7.92-7.81 (m, 3H), 7.76 (d, J = 7.3 |
| | | | Hz, 2H), 7.68-7.63 (m, 1H), 7.54-7.49 (m, 2H), 7.45-7.40 (m, |
| | | | 2H), 7.37-7.33 (m, 1H), 5.08 (s, 1H), 4.26-4.16 (m, 3H), 3.85- |
| | | | 3.78 (m, 1H), 3.76-3.69 (m, 2H), 3.19-3.10 (m, 1H), 2.95-2.84 |
| | | | (m, 1H), 2.83-2.73 (m, 1H), 2.37-2.32 (m, 2H), 2.08-2.00 (m, |
| | | | 1H), 1.96-1.90 (m, 2H), 1.81-1.74 (m, 2H), 1.68-1.60 (m, 1H), |
| | | | 1.58-1.35 (m, 11H) |
| 75 | 880 | 88 | δ 11.14 (s, 1H), 10.43 (s, 1H), 8.82 (s, 1H), 8.78 (s, 1H), 8.72 (s, |
| | | | 2H), 8.18 (s, 1H), 7.90 (t, J = 7.8 Hz, 2H), 7.85 (d, J = 8.3 Hz, 1H), |
| | | | 7.78 (d, J = 7.2 Hz, 2H), 7.67 (t, J = 7.8 Hz, 1H), 7.57-7.49 (m, |
| | | | 2H), 7.48-7.41 (m, 2H), 7.37 (dd, J = 8.3, 2.2 Hz, 1H), 5.14 (dd, J = |
| | | | 12.8, 5.3 Hz, 1H), 4.19 (br s, 3H), 3.84 (br d, J = 13.6 Hz, 1H), 3.75 |
| | | | (br t, J = 6.5 Hz, 2H), 3.16 (br t, J = 11.6 Hz, 1H), 2.79 (br s, 2H), |
| | | | 2.67-2.55 (m, 2H), 2.43-2.34 (m, 2H), 2.10-2.02 (m, 1H), 1.95 |
| | | | (br s, 2H), 1.85-1.77 (m, 2H), 1.67-1.57 (m, 3H), 1.54-1.44 (m, 5H) |
| 76 | 866 | 867 | δ 11.12 (s, 1H), 10.43 (br s, 1H), 8.81 (br s, 1H), 8.76 (s, 1H), 8.71 |
| | | | (br s, 2H), 8.16 (s, 1H), 7.92-7.81 (m, 3H), 7.76 (d, J = 7.4 Hz, 2H), |
| | | | 7.70-7.62 (m, 1H), 7.55-7.48 (m, 2H), 7.48-7.40 (m, 2H), 7.36 |
| | | | (dd, J = 8.3, 2.1 Hz, 1H), 5.12 (dd, J = 12.9, 5.3 Hz, 1H), 4.22 (br t, |
| | | | J = 6.1 Hz, 3H), 3.84 (br d, J = 14.3 Hz, 1H), 3.74 (br t, J = 6.4 Hz, |
| | | | 2H), 3.15 (br t, J = 11.8 Hz, 1H), 2.97-2.76 (m, 2H), 2.66-2.54 (m, |
| | | | 2H), 2.44 (br d, J = 3.1 Hz, 2H), 2.10-2.01 (m, 1H), 1.95 (br t, J = |
| | | | 6.1 Hz, 2H), 1.86-1.75 (m, 2H), 1.74-1.60 (m, 3H), 1.59-1.40 (m, 3H) |
| 77 | 965 | 966 | δ 11.11 (s, 1H), 10.24 (s, 1H), 8.75-8.70 (m, 2H), 8.64-8.60 (m, |
| | | | 2H), 8.16 (s, 1H), 7.94-7.86 (m, 3H), 7.82 (s, 1H), 7.76 (br d, J = |
| | | | 7.3 Hz, 2H), 7.65 (t, J = 7.7 Hz, 1H), 7.54-7.49 (m, 3H), 7.43-7.38 |
| | | | (m, 2H), 5.17-5.08 (m, 1H), 4.77 (s, 2H), 4.25-4.16 (m, 1H), 3.85- |
| | | | 3.77 (m, 1H), 3.72 (br t, J = 6.6 Hz, 2H), 3.18-3.11 (m, 3H), 2.96- |
| | | | 2.84 (m, 1H), 2.07-2.00 (m, 1H), 1.93 (br t, J = 6.1 Hz, 2H), 1.69- |
| | | | 1.58 (m, 2H), 1.52-1.40 (m, 8H), 1.33-1.25 (m, 7H) |
| 78 | 922 | 923 | δ 10.24 (s, 1H), 8.73 (s, 2H), 8.62 (dd, J = 8.3, 2.0 Hz, 2H), 8.16 (s, |
| | | | 1H), 7.91-7.85 (m, 2H), 7.82-7.74 (m, 4H), 7.65 (t, J = 7.8 Hz, |
| | | | 1H), 7.51 (d, J = 8.0 Hz, 3H), 7.44-7.40 (m, 2H), 5.09-5.00 (m, |
| | | | 1H), 4.21 (br t, J = 6.3 Hz, 3H), 3.86-3.77 (m, 1H), 3.72 (br t, J = |
| | | | 6.7 Hz, 2H), 3.17-3.09 (m, 1H), 2.88-2.74 (m, 2H), 2.35-2.32 (m, |
| | | | 3H), 2.07-1.97 (m, 2H), 1.95-1.90 (m, 2H), 1.82-1.72 (m, 3H), |
| | | | 1.66-1.60 (m, 1H), 1.48 (br dd, J = 15.0, 11.1 Hz, 7H), 1.33 (br s, 5H) |
| 79 | 908 | 909 | δ 11.10-11.06 (m, 1H), 10.24 (s, 1H), 8.73 (s, 2H), 8.63-8.58 (m, |
| | | | 2H), 8.16 (s, 1H), 7.93-7.85 (m, 2H), 7.83-7.73 (m, 3H), 7.65 (t, |
| | | | J = 7.7 Hz, 1H), 7.54-7.49 (m, 3H), 7.46-7.39 (m, 2H), 5.11-5.00 |
| | | | (m, 1H), 4.25-4.17 (m, 3H), 3.85-3.77 (m, 1H), 3.72 (br t, J = 6.7 |
| | | | Hz, 2H), 2.87-2.75 (m, 5H), 2.07-1.99 (m, 1H), 1.93 (br t, J = 6.1 |
| | | | Hz, 2H), 1.82-1.73 (m, 2H), 1.70-1.59 (m, 2H), 1.57-1.41 (m, |
| | | | 8H), 1.39-1.30 (m, 4H) |
| 80 | 894 | 895 | δ 11.10 (s, 1H), 10.25 (s, 1H), 8.73 (s, 2H), 8.63 (br d, J = 8.3 Hz, |
| | | | 2H), 8.17-8.13 (m, 1H), 7.92-7.84 (m, 2H), 7.83-7.73 (m, 3H), |
| | | | 7.65 (t, J = 7.8 Hz, 1H), 7.55-7.48 (m, 3H), 7.46-7.39 (m, 2H), |
| | | | 5.12-5.05 (m, 1H), 4.21 (br t, J = 6.3 Hz, 3H), 3.71 (br t, J = 6.5 Hz, |
| | | | 2H), 3.13 (br t, J = 11.6 Hz, 1H), 2.94-2.74 (m, 2H), 2.62-2.54 (m, |
| | | | 2H), 2.38-2.29 (m, 2H), 2.07-1.98 (m, 1H), 1.93 (br t, J = 6.3 Hz, |
| | | | 2H), 1.81-1.73 (m, 2H), 1.69-1.59 (m, 1H), 1.58-1.34 (m, 10H) |
| 81 | 856 | 857 | δ 11.11 (br s, 1H), 10.14 (s, 1H), 10.06 (s, 1H), 8.70 (s, 1H), 8.64 (d, |
| | | | J = 2.3 Hz, 1H), 8.56-8.53 (m, 1H), 8.37 (d, J = 2.0 Hz, 1H), 8.14- |
| | | | 8.10 (m, 1H), 7.91-7.78 (m, 4H), 7.76-7.70 (m, 2H), 7.68-7.62 |
| | | | (m, 1H), 7.54-7.48 (m, 3H), 7.47-7.38 (m, 2H), 5.08 (dd, J = 12.8, |
| | | | 5.4 Hz, 1H), 4.20 (t, J = 6.4 Hz, 2H), 3.04 (q, J = 6.6 Hz, 2H), 2.94- |
| | | | 2.83 (m, 1H), 2.63-2.55 (m, 1H), 2.38-2.20 (m, 5H), 2.11-1.92 |
| | | | (m, 3H), 1.61-1.52 (m, 2H), 1.45-1.34 (m, 2H), 1.27 (br s, 6H) |
| 82 | 865 | 866 | δ 10.92 (s, 1H), 10.25 (s, 1H), 8.73 (s, 2H), 8.62 (dd, J = 5.6, 2.0 Hz, |
| | | | 2H), 8.16 (s, 1H), 7.87 (br d, J = 9.3 Hz, 2H), 7.76 (d, J = 7.3 Hz, |
| | | | 2H), 7.65 (t, J = 7.8 Hz, 1H), 7.54-7.49 (m, 2H), 7.47-7.36 (m, |
| | | | 2H), 6.68-6.61 (m, 2H), 6.40-6.32 (m, 1H), 5.01 (dd, J = 13.1, 5.2 |
| | | | Hz, 1H), 4.30-4.20 (m, 2H), 4.17-4.11 (m, 1H), 3.87-3.78 (m, |
| | | | 1H), 3.72 (br t, J = 6.7 Hz, 2H), 3.18-3.05 (m, 3H), 2.95-2.76 (m, |
| | | | 2H), 2.38-2.33 (m, 3H), 1.94 (br d, J = 7.1 Hz, 3H), 1.66-1.52 (m, |
| | | | 6H), 1.49-1.37 (m, 5H) |
| 83 | 856 | 857 | δ 11.12-11.06 (m, 1H), 11.01-10.99 (m, 1H), 10.14 (s, 1H), 10.05 |
| | | | (s, 1H), 8.69 (s, 1H), 8.63 (d, J = 2.1 Hz, 2H), 8.55 (br d, J = 2.1 Hz, |
| | | | 1H), 8.36 (d, J = 1.9 Hz, 1H), 8.13-8.09 (m, 2H), 7.90-7.85 (m, |
| | | | 2H), 7.67-7.63 (m, 2H), 7.52-7.47 (m, 5H), 7.44-7.41 (m, 3H), |
| | | | 5.06 (d, J = 5.4 Hz, 1H), 4.18 (s, 2H), 3.04-3.00 (m, 2H), 2.93- |
| | | | 2.83 (m, 2H), 2.59-2.54 (m, 1H), 2.31-2.28 (m, 2H), 2.10-2.05 |
| | | | (m, 3H), 1.75 (br d, J = 7.6 Hz, 2H), 1.55 (br d, J = 6.8 Hz, 3H), 1.43- |
| | | | 1.40 (m, 4H), 1.31-1.27 (m, 2H) |
| 84 | 851 | 852 | δ 10.92 (s, 1H), 10.26 (s, 1H), 8.76-8.72 (m, 2H), 8.65-8.61 (m, |
| | | | 2H), 8.20-8.16 (m, 1H), 7.93-7.86 (m, 2H), 7.80-7.76 (m, 2H), |
| | | | 7.71-7.63 (m, 1H), 7.55-7.50 (m, 2H), 7.47-7.38 (m, 2H), 6.71- |
| | | | 6.64 (m, 2H), 6.40 (t, J = 5.4 Hz, 1H), 5.03 (dd, J = 13.3, 5.1 Hz, |
| | | | 1H), 4.33-4.21 (m, 2H), 4.18-4.13 (m, 1H), 3.84 (br d, J = 14.3 |
| | | | Hz, 1H), 3.74 (br t, J = 6.8 Hz, 2H), 3.19-3.09 (m, 3H), 2.97-2.79 |
| | | | (m, 2H), 2.43-2.31 (m, 4H), 1.95 (br t, J = 6.5 Hz, 3H), 1.63 (br s, |
| | | | 4H), 1.57-1.40 (m, 4H) |
| 85 | 856 | 857 | δ 11.12 (s, 1H), 10.13 (s, 1H), 10.04 (s, 1H), 8.69 (s, 1H), 8.63 (d, |
| | | | J = 2.3 Hz, 1H), 8.57-8.53 (m, 1H), 8.36 (d, J = 1.9 Hz, 1H), 8.13- |
| | | | 8.10 (m, 1H), 7.94-7.78 (m, 4H), 7.75-7.70 (m, 2H), 7.68-7.62 |
| | | | (m, 1H), 7.53-7.47 (m, 3H), 7.44-7.37 (m, 2H), 5.12 (m, 1H), 4.77 |
| | | | (s, 2H), 3.14 (m, 2H), 2.95-2.85 (m, 1H), 2.62 (br d, J = 2.6 Hz, |
| | | | 1H), 2.59-2.53 (m, 1H), 2.34-2.27 (m, 2H), 2.08-2.01 (m, 1H), |
| | | | 1.57 (br t, J = 6.9 Hz, 2H), 1.47-1.39 (m, 2H), 1.26 (br s, 10H) |
| 86 | 828 | 829 | δ 11.12 (s, 1H), 10.14 (s, 1H), 10.05 (s, 1H), 8.69 (s, 1H), 8.63 (d, |
| | | | J = 2.1 Hz, 1H), 8.54 (s, 1H), 8.36 (d, J = 1.5 Hz, 1H), 8.11 (t, J = 1.6 |
| | | | Hz, 1H), 7.94 (t, J = 5.7 Hz, 1H), 7.91-7.83 (m, 2H), 7.80 (m, 1H), |
| | | | 7.75-7.71 (m, 2H), 7.68-7.62 (m, 1H), 7.53-7.47 (m, 3H), 7.44- |
| | | | 7.37 (m, 2H), 5.12 (m, 1H), 4.77 (s, 2H), 3.15 (m, 2H), 2.95-2.84 |
| | | | (m, 1H), 2.69-2.53 (m, 2H), 2.34-2.27 (m, 2H), 2.07-2.00 (m, |
| | | | 1H), 1.61-1.53 (m, 2H), 1.48-1.39 (m, 2H), 1.28 (br s, 6H) |
| 87 | 852 | 853 | δ 10.96 (s, 1H), 10.36 (s, 1H), 8.80-8.74 (m, 2H), 8.68 (br s, 2H), |
| | | | 8.16 (s, 1H), 7.89 (t, J = 8.5 Hz, 2H), 7.76 (d, J = 7.4 Hz, 2H), 7.68- |
| | | | 7.60 (m, 2H), 7.54-7.48 (m, 2H), 7.46-7.40 (m, 1H), 7.17 (s, 1H), |
| | | | 7.06 (m, 1H), 5.07 (m, 1H), 4.41 (s, 1H), 4.42-4.35 (m, 1H), 4.29- |
| | | | 4.18 (m, 2H), 4.10 (t, J = 6.3 Hz, 2H), 3.83 (br d, J = 13.7 Hz, 1H), |
| | | | 3.73 (br t, J = 6.7 Hz, 3H), 2.96-2.76 (m, 2H), 2.59 (br d, J = 16.7 |
| | | | Hz, 1H), 2.46-2.31 (m, 3H), 2.02-1.91 (m, 3H), 1.83-1.75 (m, |
| | | | 2H), 1.74-1.59 (m, 3H), 1.57-1.40 (m, 3H) |
| 88 | 866 | 867 | δ10.97 (br d, J = 1.6 Hz, 1H), 10.25 (s, 1H), 8.73 (s, 2H), 8.63 (dd, |
| | | | J = 3.8, 2.1 Hz, 2H), 8.16 (s, 1H), 7.89 (br t, J = 9.3 Hz, 2H), 7.77 (d, |
| | | | J = 7.5 Hz, 2H), 7.67-7.60 (m, 2H), 7.55-7.49 (m, 2H), 7.44 (d, J = |
| | | | 7.4 Hz, 1H), 7.17 (s, 1H), 7.05 (dd, J = 8.4, 1.9 Hz, 1H), 5.08 (dd, |
| | | | J = 13.1, 5.1 Hz, 1H), 4.44-4.34 (m, 1H), 4.26 (br d, J = 17.1 Hz, |
| | | | 2H), 4.08 (br t, J = 6.4 Hz, 2H), 3.88-3.78 (m, 1H), 3.72 (br t, J = |
| | | | 6.3 Hz, 2H), 3.14 (br s, 1H), 2.93-2.70 (m, 3H), 2.44-2.34 (m, |
| | | | 3H), 1.99-1.87 (m, 3H), 1.84-1.73 (m, 2H), 1.70-1.54 (m, 4H), |
| | | | 1.52-1.44 (m, 4H) |
| 89 | 856 | 857 | δ 11.10 (s, 1H), 10.14 (s, 1H), 10.05 (s, 1H), 8.70 (s, 1H), 8.64 (br s, |
| | | | 1H), 8.55 (br s, 1H), 8.37 (br s, 1H), 8.12 (s, 1H), 7.95 (br t, J = 5.4 |
| | | | Hz, 1H), 7.87 (br dd, J = 13.8, 8.0 Hz, 2H), 7.83-7.78 (m, 1H), 7.73 |
| | | | (d, J = 7.3 Hz, 2H), 7.65 (t, J = 7.8 Hz, 1H), 7.55-7.49 (m, 3H), |
| | | | 7.47-7.39 (m, 2H), 5.07 (dd, J = 12.7, 5.2 Hz, 1H), 4.41 (br t, J = |
| | | | 6.1 Hz, 2H), 3.10-3.02 (m, 2H), 2.93-2.83 (m, 1H), 2.60 (br t, J = |
| | | | 6.0 Hz, 3H), 2.29 (br t, J = 7.3 Hz, 2H), 2.06-1.98 (m, 1H), 1.56 (br |
| | | | d, J = 6.4 Hz, 2H), 1.44-1.36 (m, 2H), 1.25 (br s, 9H) |
| 91 | 726 | 727 | δ 10.76 (s, 1H), 9.64 (s, 1H), 8.68-8.63 (m, 1H), 8.58 (d, J = 2.0 |
| | | | Hz, 1H), 8.43 (d, J = 2.1 Hz, 1H), 8.11 (s, 1H), 6.77 (d, J = 8.8 Hz, |
| | | | 2H), 6.60 (d, J = 9.0 Hz, 2H), 5.37 (d, J = 7.3 Hz, 1H), 4.19 (ddd, J = |
| | | | 11.3, 6.8, 5.0 Hz, 1H), 3.81 (t, J = 6.9 Hz, 2H), 3.61 (br s, 4H), 3.44 |
| | | | (br d, J = 11.3 Hz, 2H), 2.91-2.84 (m, 2H), 2.76-2.68 (m, 1H), |
| | | | 2.60 (br t, J = 4.4 Hz, 1H), 2.34-2.25 (m, 3H), 2.14-2.04 (m, 3H), |
| | | | 1.89-1.71 (m, 6H), 1.64-1.49 (m, 11H) |
| 166 | 880 | 881 | δ 10.23 (s, 1H), 8.73 (s, 1H), 8.69-8.64 (m, 2H), 8.52 (d, J = 2.1 |
| | | | Hz, 1H), 8.12 (s, 1H), 7.87 (t, J = 8.4 Hz, 2H), 7.76-7.72 (m, 2H), |
| | | | 7.69-7.57 (m, 2H), 7.56-7.46 (m, 3H), 7.45-7.38 (m, 1H), 7.16 |
| | | | (d, J = 1.8 Hz, 1H), 7.04 (dd, J = 8.2, 2.2 Hz, 1H), 5.17-4.98 (m, |
| | | | 1H), 4.46-4.32 (m, 1H), 4.30-4.22 (m, 1H), 4.07 (t, J = 6.4 Hz, |
| | | | 2H), 3.65 (s, 2H), 3.52-3.41 (m, 1H), 2.98-2.80 (m, 1H), 2.36- |
| | | | 2.25 (m, 4H), 2.08-1.90 (m, 2H), 1.84-1.64 (m, 3H), 1.60-1.33 |
| | | | (m, 13H) |
| 167 | 878 | 879 | δ 11.10 (s, 1 H) 10.65-10.34 (m, 2 H) 8.92-8.71 (m, 2 H) |
| | | | 8.66-8.50 (m, 3 H) 8.26-8.04 (m, 2 H) 7.93-7.79 (m, 3 H) |
| | | | 7.73 (br d, J = 7.5 Hz, 2 H) 7.69-7.62 (m, 1 H) 7.50 (t, J = 7.6 |
| | | | Hz, 2 H) 7.45-7.36 (m, 1 H) 7.02 (d, J = 7.9 Hz, 1 H) 6.83 (d, |
| | | | J = 8.00 Hz, 1 H) 5.48-5.36 (m, 1 H) 4.64 (s, 2 H) 3.17 (br d, |
| | | | J = 6.8 Hz, 2 H) 3.04-2.87 (m, 2 H) 2.81-2.59 (m, 2 H) 2.33 |
| | | | (br t, J = 7.1 Hz, 2 H) 1.57 (br s, 2 H) 1.51-1.39 (m, 2 H) 1.25 |
| | | | (br s, 10 H) |
| 168 | 768 | 769 | δ 10.76 (br s, 1H), 9.66 (s, 1H), 8.64 (t, J = 2.1 Hz, 1H), 8.57 (d, J = |
| | | | 2.1 Hz, 1H), 8.43 (d, J = 2.3 Hz, 1H), 8.10 (s, 1H), 6.75 (d, J = 8.9 |
| | | | Hz, 2H), 6.59 (d, J = 9.0 Hz, 2H), 5.36 (d, J = 7.3 Hz, 1H), 4.31- |
| | | | 4.22 (m, 1H), 4.18 (ddd, J = 11.3, 6.9, 4.7 Hz, 1H), 3.90 (br d, J = |
| | | | 4.6 Hz, 1H), 3.85 (br t, J = 6.9 Hz, 2H), 3.59 (br s, 5H), 3.27-3.19 |
| | | | (m, 1H), 2.96-2.86 (m, 2H), 2.78-2.66 (m, 1H), 2.47-2.38 (m, |
| | | | 1H), 2.31 (br t, J = 5.9 Hz, 2H), 2.17-2.08 (m, 3H), 1.79-1.67 (m, |
| | | | 5H), 1.65-1.51 (m, 11H), 1.38-1.26 (m, 2H) |
| 169 | 784 | 785 | δ 10.77 (s, 1H), 9.65 (s, 1H), 8.62-8.55 (m, 2H), 8.50 (d, J = 2.0 |
| | | | Hz, 1H), 8.19 (s, 1H), 8.10 (s, 1H), 6.95 (d, J = 8.5 Hz, 2H), 6.61 (d, |
| | | | J = 8.6 Hz, 2H), 5.65 (br d, J = 7.3 Hz, 1H), 5.15-4.67 (m, 1H), |
| | | | 4.30-4.19 (m, 2H), 4.15-4.03 (m, 2H), 4.00-3.92 (m, 1H), 3.87 |
| | | | (br s, 2H), 3.59 (dt, J = 8.4, 4.5 Hz, 1H), 3.27-3.19 (m, 2H), 3.12 |
| | | | (br d, J = 12.4 Hz, 2H), 2.99 (br dd, J = 12.2, 9.1 Hz, 1H), 2.92 (br d, |
| | | | J = 11.0 Hz, 3H), 2.79-2.67 (m, 1H), 2.60 (br t, J = 4.1 Hz, 1H), |
| | | | 2.38-2.29 (m, 1H), 2.17 (br t, J = 6.9 Hz, 2H), 2.14-2.06 (m, 3H), |
| | | | 1.94-1.86 (m, 2H), 1.84-1.77 (m, 2H), 1.71 (br d, J = 1.1 Hz, 2H), |
| | | | 1.57 (br s, 6H), 1.45-1.38 (m, 1H) |
| 170 | 784 | 785 | δ 10.82-10.77 (m, 1H), 9.71-9.65 (m, 1H), 8.63-8.55 (m, 2H), |
| | | | 8.54-8.47 (m, 1H), 8.15 (s, 1H), 8.12 (s, 1H), 7.02-6.92 (m, 2H), |
| | | | 6.58 (s, 2H), 5.75-5.65 (m, 1H), 4.85 (s, 1H), 4.35-4.19 (m, 2H), |
| | | | 4.18-4.06 (m, 1H), 4.04-3.91 (m, 2H), 3.91-3.81 (m, 2H), 3.69- |
| | | | 3.46 (m, 2H), 3.30-3.19 (m, 2H), 3.18-3.07 (m, 2H), 3.06-2.85 |
| | | | (m, 3H), 2.85-2.66 (m, 2H), 2.64-2.58 (m, 1H), 2.40-2.30 (m, |
| | | | 1H), 2.23-2.16 (m, 2H), 2.05 (br d, J = 3.8 Hz, 2H), 1.98-1.71 (m, |
| | | | 7H), 1.69-1.48 (m, 5H), 1.47-1.35 (m, 1H) |
| 171 | 836 | 837 | δ 10.80 (s, 1H), 9.74 (s, 1H), 8.61 (br d, J = 6.2 Hz, 2H), 8.48 (s, |
| | | | 1H), 8.20-8.18 (m, 1H), 8.16 (s, 1H), 6.96 (d, J = 8.3 Hz, 2H), 6.62 |
| | | | (d, J = 8.3 Hz, 2H), 5.68 (d, J = 7.5 Hz, 1H), 4.46-4.37 (m, 1H), |
| | | | 4.32-4.22 (m, 3H), 4.04 (br d, J = 12.3 Hz, 1H), 3.87 (br t, J = 6.8 |
| | | | Hz, 2H), 3.30-3.20 (m, 3H), 3.08-3.01 (m, 2H), 2.99 (br s, 2H), |
| | | | 2.98-2.87 (m, 2H), 2.81-2.70 (m, 1H), 2.67-2.56 (m, 2H), 2.43- |
| | | | 2.34 (m, 1H), 2.26-2.16 (m, 4H), 2.15-2.07 (m, 1H), 2.03-1.95 |
| | | | (m, 1H), 1.92-1.79 (m, 3H), 1.74 (br s, 2H), 1.65-1.56 (m, 6H) |
| 172 | 836 | 837 | δ 10.80 (s, 1H), 9.74 (s, 1H), 8.61 (br d, J = 6.4 Hz, 2H), 8.48 (s, |
| | | | 1H), 8.19 (s, 1H), 8.17 (s, 1H), 6.96 (br d, J = 8.2 Hz, 2H), 6.62 (br |
| | | | d, J = 8.3 Hz, 2H), 5.68 (br d, J = 7.2 Hz, 1H), 4.41 (br d, J = 12.3 |
| | | | Hz, 1H), 4.30-4.22 (m, 3H), 4.05 (br d, J = 10.9 Hz, 1H), 3.87 (br t, |
| | | | J = 6.7 Hz, 2H), 3.39 (br d, J = 13.2 Hz, 1H), 3.27-3.19 (m, 2H), |
| | | | 3.08-2.99 (m, 3H), 2.97 (br s, 2H), 2.94-2.87 (m, 1H), 2.80-2.70 |
| | | | (m, 1H), 2.65-2.56 (m, 2H), 2.42-2.32 (m, 1H), 2.18 (br t, J = 6.6 |
| | | | Hz, 4H), 2.14-2.07 (m, 1H), 1.99 (br d, J = 10.1 Hz, 1H), 1.91- |
| | | | 1.79 (m, 3H), 1.74 (br s, 2H), 1.64-1.55 (m, 6H) |
| 173 | 754 | 755 | δ 10.76 (s, 1H), 9.64 (s, 1H), 8.63 (t, J = 2.2 Hz, 1H), 8.57 (d, J = 2.1 |
| | | | Hz, 1H), 8.43 (d, J = 2.1 Hz, 1H), 8.18-8.14 (m, 1H), 8.11 (s, 1H), |
| | | | 6.75 (d, J = 9.0 Hz, 2H), 6.60 (d, J = 8.9 Hz, 2H), 5.36 (d, J = 7.3 |
| | | | Hz, 1H), 4.23-4.15 (m, 1H), 3.81 (br t, J = 6.9 Hz, 2H), 3.61 (br s, |
| | | | 4H), 3.52-3.48 (m, 1H), 2.82 (br d, J = 11.3 Hz, 2H), 2.74-2.66 |
| | | | (m, 1H), 2.62-2.53 (m, 2H), 2.37-2.32 (m, 2H), 2.10-1.98 (m, |
| | | | 6H), 1.75 (br d, J = 12.1 Hz, 4H), 1.63 (br s, 6H), 1.50 (br d, J = 12.0 |
| | | | Hz, 4H), 1.45-1.39 (m, 2H), 1.32-1.25 (m, 2H) |
| 174 | 767 | 768 | δ 10.83-10.68 (m, 1H), 10.26-10.21 (m, 1H), 8.80-8.75 (m, 1H), |
| | | | 8.73-8.69 (m, 1H), 8.63 (d, J = 1.3 Hz, 1H), 8.54-8.48 (m, 1H), |
| | | | 7.95 (br d, J = 8.2 Hz, 2H), 7.66-7.59 (m, 2H), 6.79-6.72 (m, 2H), |
| | | | 6.65-6.57 (m, 2H), 5.39-5.33 (m, 1H), 4.24-4.14 (m, 1H), 3.85- |
| | | | 3.77 (m, 2H), 2.84-2.78 (m, 2H), 2.66 (br d, J = 1.5 Hz, 2H), 2.41- |
| | | | 2.32 (m, 1H), 2.23 (br s, 1H), 2.23-2.22 (m, 1H), 2.24-2.17 (m, |
| | | | 1H), 2.15-2.12 (m, 1H), 2.11-2.09 (m, 1H), 2.11-2.01 (m, 5H), |
| | | | 1.91-1.75 (m, 6H), 1.65-1.56 (m, 1H), 1.55-1.48 (m, 2H), 1.30- |
| | | | 1.16 (m, 3H) |
| 175 | 784 | 785 | δ 10.77 (br s, 1H), 9.65 (s, 1H), 8.59 (d, J = 2.0 Hz, 1H), 8.56 (t, J = |
| | | | 2.2 Hz, 1H), 8.50 (d, J = 2.0 Hz, 1H), 8.11 (s, 1H), 6.99-6.71 (m, |
| | | | 2H), 6.65 (br d, J = 1.9 Hz, 2H), 5.60-5.17 (m, 1H), 4.89 (br d, J = |
| | | | 4.0 Hz, 1H), 4.27 (br d, J = 12.9 Hz, 2H), 4.12 (br dd, J = 13.0, 3.4 |
| | | | Hz, 1H), 4.00-3.91 (m, 2H), 3.88 (br d, J = 4.0 Hz, 2H), 3.60 (dt, |
| | | | J = 8.4, 4.4 Hz, 1H), 3.40 (br s, 2H), 3.25 (br d, J = 12.4 Hz, 2H), 3.16- |
| | | | 3.09 (m, 1H), 2.99 (dd, J = 12.5, 8.8 Hz, 1H), 2.94-2.86 (m, 1H), |
| | | | 2.77-2.69 (m, 1H), 2.61 (br s, 1H), 2.34 (br d, J = 2.0 Hz, 2H), 2.16 |
| | | | (br t, J = 6.9 Hz, 3H), 2.11 (br dd, J = 4.9, 3.4 Hz, 1H), 1.96-1.89 |
| | | | (m, 2H), 1.80 (br dd, J = 9.1, 4.8 Hz, 2H), 1.76-1.67 (m, 2H), 1.64- |
| | | | 1.47 (m, 6H), 1.43-1.39 (m, 1H) |
| 176 | 784 | 785 | δ 10.91-10.79 (m, 1H), 10.66-10.41 (m, 1H), 9.97-9.75 (m, 1H), |
| | | | 8.70 (s, 1H), 8.63 (br s, 2H), 8.20-8.09 (m, 1H), 7.42 (br d, J = 8.1 |
| | | | Hz, 2H), 6.78 (br d, J = 9.0 Hz, 2H), 6.56-6.17 (m, 1H), 4.45-4.37 |
| | | | (m, 1H), 4.29-4.23 (m, 1H), 4.18-4.05 (m, 2H), 4.00-3.93 (m, |
| | | | 2H), 3.91-3.85 (m, 3H), 3.62-3.57 (m, 4H), 3.21-3.11 (m, 3H), |
| | | | 3.08-2.99 (m, 2H), 2.97-2.87 (m, 2H), 2.82-2.70 (m, 2H), 2.45- |
| | | | 2.38 (m, 2H), 2.19 (br t, J = 6.4 Hz, 3H), 1.92 (br dd, J = 12.1, 4.3 |
| | | | Hz, 2H), 1.86-1.77 (m, 2H), 1.59 (br d, J = 4.9 Hz, 4H), 1.48-1.36 |
| | | | (m, 2H) |
| 177 | 836 | 837 | δ 8.82 (br d, J = 7.9 Hz, 1H), 8.68 (br s, 1H), 8.54 (d, J = 2.0 Hz, |
| | | | 1H), 8.39 (br d, J = 4.0 Hz, 1H), 8.10 (s, 1H), 7.40 (br s, 1H), 6.99- |
| | | | 6.91 (m, 2H), 6.69 (br d, J = 8.7 Hz, 2H), 4.58 (br d, J = 13.0 Hz, |
| | | | 1H), 4.46 (br d, J = 13.0 Hz, 1H), 4.37 (br d, J = 13.3 Hz, 1H), 4.04 |
| | | | (br dd, J = 12.3, 4.6 Hz, 1H), 3.99-3.86 (m, 3H), 3.76-3.57 (m, |
| | | | 1H), 3.50 (br d, J = 10.5 Hz, 2H), 3.34 (br t, J = 10.8 Hz, 1H), 3.22 |
| | | | (br t, J = 10.8 Hz, 1H), 2.97-2.85 (m, 3H), 2.80-2.68 (m, 3H), 2.58- |
| | | | 2.47 (m, 2H), 2.36 (br t, J = 7.3 Hz, 2H), 2.20 (br dd, J = 11.1, 6.7 |
| | | | Hz, 2H), 2.13 (br d, J = 11.5 Hz, 2H), 2.03-1.97 (m, 2H), 1.95- |
| | | | 1.89 (m, 4H), 1.66-1.54 (m, 5H) |
| 178 | 836 | 837 | δ 8.73 (br d, J = 7.6 Hz, 1H), 8.43 (br s, 1H), 8.30 (br d, J = 4.5 Hz, |
| | | | 1H), 8.25-8.18 (m, 1H), 8.00 (s, 1H), 7.31 (br d, J = 8.6 Hz, 1H), |
| | | | 7.13 (br s, 1H), 6.91-6.82 (m, 2H), 6.60 (br d, J = 8.6 Hz, 2H), 4.48 |
| | | | (br d, J = 12.5 Hz, 1H), 4.41-4.32 (m, 1H), 4.31-4.23 (m, 1H), |
| | | | 3.95 (br dd, J = 12.1, 4.3 Hz, 1H), 3.90-3.78 (m, 3H), 3.62-3.53 |
| | | | (m, 1H), 3.42 (br d, J = 10.1 Hz, 2H), 3.25 (br t, J = 11.6 Hz, 1H), |
| | | | 3.13 (br t, J = 10.6 Hz, 1H), 2.89-2.76 (m, 3H), 2.71-2.60 (m, 3H), |
| | | | 2.50-2.38 (m, 2H), 2.29-2.22 (m, 2H), 2.11 (br dd, J = 11.1, 6.2 |
| | | | Hz, 2H), 2.07-2.00 (m, 2H), 1.90 (br d, J = 11.9 Hz, 2H), 1.83 (br |
| | | | d, J = 12.8 Hz, 4H), 1.57-1.48 (m, 5H) |
| 179 | 830 | 831 | δ 10.84-10.70 (m, 1H), 10.15 (s, 1H), 8.73 (d, J = 2.1 Hz, 1H), 8.68- |
| | | | 8.62 (m, 2H), 8.57-8.53 (m, 1H), 7.31 (t, J = 7.9 Hz, 1H), 7.05 (d, |
| | | | J = 8.0 Hz, 1H), 6.94 (td, J = 2.0, 4.3 Hz, 3H), 6.69 (dd, J = 2.1, 8.2 |
| | | | Hz, 1H), 6.60 (d, J = 8.5 Hz, 2H), 5.59 (s, 1H), 4.33-4.16 (m, 2H), |
| | | | 4.11-3.93 (m, 1H), 3.90-3.77 (m, 2H), 3.29-3.25 (m, 5H), 3.07- |
| | | | 2.86 (m, 3H), 2.79-2.65 (m, 2H), 2.63-2.53 (m, 2H), 2.46-2.31 |
| | | | (m, 2H), 2.22-2.03 (m, 4H), 2.02-1.93 (m, 4H), 1.90-1.51 (m, 9H) |
| 180 | 853 | 854 | δ ppm 10.75 (s, 1H), 10.24 (s, 1H), 8.73 (s, 2H), 8.62 (dd, J = 2.1, |
| | | | 5.8 Hz, 2H), 8.16 (s, 1H), 7.88 (dd, J = 8.0, 13.3 Hz, 2H), 7.77-7.73 |
| | | | (m, 2H), 7.65 (t, J = 7.8 Hz, 1H), 7.54-7.48 (m, 2H), 7.45-7.40 (m, |
| | | | 1H), 6.76 (d, J = 8.9 Hz, 2H), 6.60 (d, J = 8.9 Hz, 2H), 5.33 (br d, J = |
| | | | 7.1 Hz, 1H), 4.92 (s, 1H), 4.27 (br d, J = 13.6 Hz, 1H), 4.21-4.12 |
| | | | (m, 1H), 3.97 (br d, J = 13.1 Hz, 1H), 3.72 (br t, J = 6.7 Hz, 2H), |
| | | | 3.18 (br t, J = 11.8 Hz, 2H), 3.09 (br d, J = 11.1 Hz, 2H), 2.96-2.87 |
| | | | (m, 2H), 2.86-2.80 (m, 1H), 2.77-2.66 (m, 1H), 2.62-2.57 (m, |
| | | | 1H), 2.56-2.52 (m, 2H), 2.15-2.06 (m, 1H), 1.99-1.90 (m, 2H), |
| | | | 1.88-1.78 (m, 1H), 1.77-1.70 (m, 2H), 1.69-1.62 (m, 3H), 1.60- |
| | | | 1.51 (m, 1H), 1.50-1.41 (m, 2H) |
| 181 | 846 | 847 | δ 8.83 (br s, 1H), 8.50 (s, 1H), 8.36 (dd, J = 4.6, 2.0 Hz, 1H), 8.12- |
| | | | 8.04 (m, 2H), 7.39 (br d, J = 5.2 Hz, 5H), 7.16-7.08 (m, 1H), 7.06- |
| | | | 6.99 (m, 1H), 6.72-6.63 (m, 2H), 4.65 (br d, J = 8.7 Hz, 1H), 4.57- |
| | | | 4.50 (m, 1H), 4.42 (br d, J = 13.0 Hz, 1H), 4.37-4.25 (m, 1H), 4.19- |
| | | | 4.10 (m, 1H), 4.07-3.99 (m, 1H), 3.96-3.87 (m, 2H), 3.86-3.77 |
| | | | (m, 2H), 3.58-3.44 (m, 2H), 3.37-3.25 (m, 2H), 3.23-3.13 (m, |
| | | | 1H), 3.09-2.99 (m, 1H), 2.93-2.68 (m, 4H), 2.61-2.50 (m, 1H), |
| | | | 2.43-2.28 (m, 2H), 2.18-1.99 (m, 4H), 1.98-1.82 (m, 6H), 1.51 |
| | | | (br dd, J = 7.0, 1.7 Hz, 2H) |
| 182 | 844 | 845 | δ 10.82 (br s, 1H), 10.18 (s, 1H), 8.71 (s, 1H), 8.68-8.63 (m, 2H), |
| | | | 8.60 (s, 1H), 7.37-7.35 (m, 1H), 7.34 (s, 1H), 7.24 (s, 1H), 7.22 (s, |
| | | | 1H), 7.13 (br s, 1H), 7.12-7.08 (m, 1H), 6.82-6.64 (m, 2H), 4.45- |
| | | | 4.18 (m, 2H), 3.85 (br t, J = 6.7 Hz, 5H), 3.21(br s, 7H), 2.90 (br d, |
| | | | J = 6.7 Hz, 2H), 2.17 (br t, J = 6.5 Hz, 3H), 2.05-1.85 (m, 2H), 1.83- |
| | | | 1.69 (m, 5H), 1.63 (br d, J = 4.9 Hz, 8H), 1.57 (br d, J = 3.7 Hz, 4H) |
| 183 | 798 | 799 | δ 10.82-10.70 (m, 1H), 10.37-10.25 (m, 1H), 10.20 (s, 1H), 8.73 |
| | | | (br d, J = 1.8 Hz, 2H), 8.55-8.48 (m, 1H), 8.45-8.36 (m, 1H), 8.13 |
| | | | (d, J = 8.5 Hz, 1H), 7.93 (br d, J = 2.8 Hz, 2H), 7.74 (d, J = 7.4 Hz, |
| | | | 2H), 7.70-7.63 (m, 1H), 7.52 (t, J = 7.7 Hz, 2H), 7.46-7.38 (m, |
| | | | 1H), 6.80 (s, 2H), 6.60 (d, J = 8.9 Hz, 2H), 5.43-5.33 (m, 1H), 4.13 |
| | | | (s, 1H), 3.43 (br d, J = 10.5 Hz, 2H), 2.72 (br d, J = 5.4 Hz, 2H), 2.67 |
| | | | (br d, J = 6.4 Hz, 2H), 2.60 (br d, J = 4.9 Hz, 2H), 2.56 (br s, 4H), |
| | | | 2.47 (br s, 4H), 2.34 (br s, 1H), 2.18-2.05 (m, 2H), 1.97-1.68 (m, |
| | | | 4H), 1.62-1.43 (m, 2H) |
| 184 | 830 | 831 | δ 10.90-10.59 (m, 1H), 10.15 (s, 1H), 8.72 (d, J = 2.1 Hz, 1H), 8.67 |
| | | | (s, 1H), 8.65 (s, 1H), 8.55 (d, J = 2.1 Hz, 1H), 7.30 (t, J = 7.9 Hz, |
| | | | 1H), 7.04 (d, J = 7.6 Hz, 1H), 6.95 (s, 1H), 6.92-6.49 (m, 5H), 5.59- |
| | | | 5.15 (m, 1H), 4.34-4.15 (m, 2H), 3.93-3.78 (m, 3H), 3.39 (br d, |
| | | | J = 6.8 Hz, 2H), 3.30-3.19 (m, 5H), 2.89 (br t, J = 10.2 Hz, 1H), 2.79- |
| | | | 2.53 (m, 4H), 2.33 (br s, 2H), 2.19-2.06 (m, 3H), 2.00-1.94 (m, |
| | | | 4H), 1.91-1.64 (m, 5H), 1.63-1.27 (m, 5H) |
| 185 | 844 | 845 | δ 10.77 (s, 1H), 10.18-10.14 (m, 1H), 8.72 (s, 1H), 8.67-8.61 (m, |
| | | | 2H), 8.60-8.55 (m, 1H), 7.39-7.30 (m, 2H), 7.23 (br d, J = 7.4 Hz, |
| | | | 1H), 7.10 (dd, J = 1.5, 8.3 Hz, 1H), 6.97 (br d, J = 8.3 Hz, 2H), 6.62 |
| | | | (br d, J = 8.5 Hz, 2H), 5.70 (br d, J = 7.4 Hz, 1H), 4.34-4.16 (m, |
| | | | 2H), 3.91-3.75 (m, 4H), 3.23-3.15 (m, 6H), 3.07-2.81 (m, 2H), |
| | | | 2.79-2.66 (m, 2H), 2.58 (td, J = 4.1, 17.5 Hz, 2H), 2.22-2.05 (m, |
| | | | 3H), 2.05-1.99 (m, 1H), 1.92-1.74 (m, 6H), 1.62 (br d, J = 5.0 Hz, |
| | | | 7H), 1.55 (br d, J = 4.6 Hz, 3H) |
| 186 | 846 | 847 | δ 10.77 (s, 1H), 9.73 (s, 1H), 8.61 (br s, 1H), 8.55 (d, J = 1.8 Hz, |
| | | | 1H), 8.47 (d, J = 2.0 Hz, 1H), 8.19-8.12 (m, 1H), 7.43-7.28 (m, |
| | | | 5H), 6.96 (br d, J = 8.1 Hz, 2H), 6.62 (br d, J = 8.4 Hz, 2H), 5.69 (br |
| | | | d, J = 7.3 Hz, 1H), 4.66-4.57 (m, 1H), 4.39-4.18 (m, 4H), 4.06 (br |
| | | | d, J = 11.3 Hz, 1H), 3.93-3.84 (m, 1H), 3.82-3.71 (m, 4H), 3.27- |
| | | | 3.12 (m, 5H), 3.04-2.86 (m, 3H), 2.79-2.65 (m, 2H), 2.61-2.55 |
| | | | (m, 2H), 2.13-2.00 (m, 3H), 1.91-1.69 (m, 6H), 1.62-1.41 (m, 3H) |
| 187 | 783 | 784 | δ 10.76 (s, 1H), 10.17 (s, 2H), 8.71 (s, 1H), 8.65 (d, J = 2.0 Hz, 1H), |
| | | | 8.57 (s, 1H), 8.40 (d, J = 1.3 Hz, 1H), 8.12 (s, 1H), 7.88 (dd, J = |
| | | | 15.1, 7.8 Hz, 2H), 7.76-7.72 (m, 2H), 7.69-7.63 (m, 1H), 7.52 (t, |
| | | | J = 7.6 Hz, 2H), 7.45-7.39 (m, 1H), 6.78 (br d, J = 8.5 Hz, 2H), 6.61 |
| | | | (br d, J = 8.9 Hz, 2H), 5.45-5.32 (m, 1H), 4.27-4.13 (m, 1H), 3.48- |
| | | | 3.38 (m, 3H), 2.81-2.65 (m, 3H), 2.61 (br t, J = 4.1 Hz, 2H), 2.37- |
| | | | 2.30 (m, 1H), 2.17-2.04 (m, 2H), 1.96-1.67 (m, 10H), 1.39-1.13 |
| | | | (m, 3H) |
| 188 | 816 | 817 | δ 10.76 (s, 1H), 10.16 (s, 1H), 8.93-8.60 (m, 3H), 8.56 (d, J = 1.9 |
| | | | Hz, 1H), 8.16 (s, 1H), 7.43-7.26 (m, 1H), 7.15 (br d, J = 7.5 Hz, |
| | | | 1H), 6.94 (br d, J = 8.5 Hz, 2H), 6.82 (br d, J = 1.6 Hz, 1H), 6.69- |
| | | | 6.48 (m, 3H), 5.64 (br d, J = 7.4 Hz, 1H), 4.31-4.17 (m, 2H), 4.12- |
| | | | 4.00 (m, 1H), 3.96-3.77 (m, 6H), 3.14 (br d, J = 13.3 Hz, 1H), 2.96- |
| | | | 2.86 (m, 3H), 2.82-2.64 (m, 2H), 2.61-2.55 (m, 1H), 2.38-2.29 |
| | | | (m, 3H), 2.25-1.99 (m, 6H), 1.89-1.78 (m, 2H), 1.70 (br s, 2H), |
| | | | 1.65-1.51 (m, 5H) |
| 189 | 843 | 844 | δ 10.74 (br s, 1H), 10.17 (s, 1H), 8.90-8.51 (m, 3H), 7.76-7.61 (m, |
| | | | 2H), 7.57-7.31 (m, 3H), 6.76 (br d, J = 8.0 Hz, 2H), 6.60 (br d, J = |
| | | | 8.1 Hz, 2H), 5.35 (br d, J = 2.4 Hz, 1H), 4.36-4.12 (m, 2H), 3.93- |
| | | | 3.81 (m, 3H), 3.28-3.17 (m, 2H), 2.95-2.83 (m, 1H), 2.78-2.66 |
| | | | (m, 1H), 2.65-2.56 (m, 2H), 2.35-2.28 (m, 2H), 2.18-2.08 (m, |
| | | | 3H), 1.90-1.64 (m, 12H), 1.63-1.53 (m, 3H), 1.47-1.22 (m, 8H) |
| 190 | 843 | 844 | δ 10.78 (s, 1H), 10.19 (s, 1H), 8.70 (d, J = 2.1 Hz, 1H), 8.67 (s, 1H), |
| | | | 8.65 (t, J = 2.2 Hz, 1H), 8.59 (d, J = 2.3 Hz, 1H), 7.78-7.61 (m, |
| | | | 2H), 7.53-7.32 (m, 2H), 6.97 (br d, J = 7.8 Hz, 2H), 6.65 (d, J = 8.6 |
| | | | Hz, 2H), 5.76 (br d, J = 7.5 Hz, 1H), 4.64-4.10 (m, 4H), 3.96-3.77 |
| | | | (m, 2H), 3.76-3.41 (m, 3H), 3.29-2.96 (m, 4H), 2.83-2.54 (m, |
| | | | 4H), 2.18 (br t, J = 6.8 Hz, 2H), 2.14-2.05 (m, 1H), 2.04-1.76 (m, |
| | | | 10H), 1.75-1.57 (m, 4H), 1.55-1.15 (m, 5H) |
| 191 | 829 | 830 | δ 10.83 (s, 1H), 10.66-10.54 (m, 1H), 10.35-10.26 (m, 1H), 8.75- |
| | | | 8.62 (m, 4H), 7.73-7.65 (m, 2H), 7.48-7.35 (m, 4H), 6.77 (d, J = |
| | | | 9.0 Hz, 2H), 4.43-4.23 (m, 2H), 3.94-3.82 (m, 4H), 3.24-3.04 (m, |
| | | | 3H), 2.95-2.86 (m, 1H), 2.80-2.59 (m, 2H), 2.42-2.31 (m, 3H), |
| | | | 2.17 (br t, J = 6.7 Hz, 3H), 2.10-1.92 (m, 6H), 1.83-1.57 (m, 13H) |
| 192 | 829 | 830 | δ 10.78 (s, 1H), 10.32 (s, 1H), 9.52-9.27 (m, 1H), 8.88-8.42 (m, |
| | | | 4H), 7.74-7.64 (m, 2H), 7.48-7.41 (m, 2H), 7.09-6.94 (m, 2H), |
| | | | 6.64 (br d, J = 8.5 Hz, 2H), 4.48-4.27 (m, 5H), 3.88 (br d, J = 6.9 |
| | | | Hz, 2H), 3.57 (br d, J = 10.4 Hz, 2H), 3.36-3.19 (m, 2H), 3.14- |
| | | | 2.96 (m, 4H), 2.79-2.59 (m, 3H), 2.19 (br t, J = 6.8 Hz, 2H), 2.12- |
| | | | 2.02 (m, 3H), 1.94 (br t, J = 11.8 Hz, 3H), 1.87-1.74 (m, 4H), 1.72- |
| | | | 1.53 (m, 7H) |
| 193 | 848 | 849 | δ 10.76 (s, 1H), 10.04 (s, 1H), 9.03 (br s, 1H), 8.83 (s, 1H), 8.64- |
| | | | 8.53 (m, 2H), 8.40 (s, 1H), 8.36 (d, J = 2.1 Hz, 1H), 6.77 (br d, J = |
| | | | 8.5 Hz, 2H), 6.60 (br d, J = 8.8 Hz, 2H), 5.39 (br d, J = 5.3 Hz, 1H), |
| | | | 4.67-4.48 (m, 1H), 4.42-4.27 (m, 1H), 4.25-4.12 (m, 1H), 4.08- |
| | | | 3.75 (m, 4H), 2.95-2.81 (m, 3H), 2.80-2.64 (m, 6H), 2.59 (br d, |
| | | | J = 4.1 Hz, 2H), 2.43-2.30 (m, 3H), 2.29 (br s, 8H), 1.90-1.74 (m, |
| | | | 4H), 1.72-1.58 (m, 4H), 1.41-1.21 (m, 3H) |
| 194 | 848 | 849 | δ 11.01-10.57 (m, 1H), 10.02 (s, 1H), 9.04 (br s, 1H), 8.80 (s, 1H), |
| | | | 8.65-8.47 (m, 2H), 8.42-8.31 (m, 2H), 6.92 (d, J = 8.4 Hz, 2H), |
| | | | 6.58 (d, J = 8.5 Hz, 2H), 5.63 (d, J = 7.4 Hz, 1H), 4.39-4.17 (m, |
| | | | 3H), 4.16-4.02 (m, 1H), 3.92 (br t, J = 6.9 Hz, 2H), 3.23-3.14 (m, |
| | | | 2H), 2.95-2.81 (m, 5H), 2.76-2.67 (m, 1H), 2.61-2.53 (m, 2H), |
| | | | 2.35-2.29 (m, 1H), 2.26-2.15 (m, 5H), 2.13-1.95 (m, 9H), 1.90- |
| | | | 1.77 (m, 2H), 1.75-1.46 (m, 7H) |
| 195 | 852 | 853 | δ 10.92 (s, 1H), 10.54 (br s, 1H), 8.88 (br s, 1H), 8.82-8.68 (m, |
| | | | 4H), 8.61 (s, 1H), 8.32-8.24 (m, 1H), 8.09 (br d, J = 8.0 Hz, 1H), |
| | | | 8.00-7.91 (m, 2H), 7.70 (t, J = 7.7 Hz, 1H), 7.46-7.34 (m, 2H), |
| | | | 6.75-6.60 (m, 2H), 5.01 (br dd, J = 5.0, 13.3 Hz, 1H), 4.36-4.07 |
| | | | (m, 4H), 3.80-3.75 (m, 3H), 3.21-3.04 (m, 3H), 2.96-2.74 (m, |
| | | | 2H), 2.41-2.26 (m, 3H), 2.09-1.89 (m, 3H), 1.62 (br s, 5H), 1.55- |
| | | | 1.39 (m, 4H) |
| 196 | 815 | 816 | δ 10.98 (s, 1H), 10.11 (br d, J = 5.3 Hz, 2H), 8.66 (s, 1H), 8.59 (br s, |
| | | | 2H), 8.36 (br s, 1H), 8.20 (s, 1H), 7.69-7.62 (m, 2H), 7.55 (s, 1H), |
| | | | 7.50 (s, 1H), 7.41 (br d, J = 8.1 Hz, 2H), 7.25-7.16 (m, 1H), 5.10 |
| | | | (br dd, J = 13.1, 5.1 Hz, 1H), 4.59-4.50 (m, 1H), 4.47-4.37 (m, |
| | | | 1H), 4.34-4.21 (m, 2H), 3.13-3.06 (m, 2H), 2.97-2.89 (m, 4H), |
| | | | 2.69-2.59 (m, 2H), 2.44-2.31 (m, 3H), 2.08-1.96 (m, 4H), 1.89- |
| | | | 1.77 (m, 4H), 1.72-1.62 (m, 3H), 1.57-1.47 (m, 1H), 1.02-0.96 |
| | | | (m, 2H), 0.75-0.70 (m, 2H) |
| 197 | 876 | 877 | δ 10.76 (s, 1 H), 10.03 (s, 1 H), 9.03-8.73 (m, 2 H), 8.56 (br d, J = |
| | | | 2.6 Hz, 2 H), 8.46-8.34 (m, 2 H), 8.13 (s, 1 H), 6.95 (br d, J = 8.0 |
| | | | Hz, 2 H), 6.61 (br d, J = 8.0 Hz, 2 H), 5.66 (br d, J = 7.1 Hz, 1 H), |
| | | | 4.50 (br d, J = 11.6 Hz, 1 H), 4.34-4.16 (m, 3 H), 4.03-3.76 (m, 4 |
| | | | H). 3.52 (br dd, J = 12.6, 10.8 Hz, 1 H), 3.26-3.04 (m, 6 H) 2.92 (br |
| | | | d, J = 9.5 Hz, 1 H) 2.84-2.64 (m, 2 H), 2.60-2.54 (m, 1 H), 2.46- |
| | | | 2.31 (m, 2 H), 2.21-2.01 (m, 8 H), 1.92-1.72 (m, 6 H), 1.65 (br d, |
| | | | J = 15.4 Hz, 5 H) |
| 198 | 841 | 842 | δ 10.97 (s, 1H), 10.19 (s, 1H), 8.75-8.53 (m, 4H), 8.14 (s, 0.17H), |
| | | | 7.72-7.37 (m, 6H), 7.23 (s, 1H), 5.16-5.06 (m, 1H), 4.54 (br d, J = |
| | | | 13.0 Hz, 1H), 4.47-4.38 (m, 1H), 4.33-4.12 (m, 2H), 3.81 (br t, J = |
| | | | 6.9 Hz, 2H), 3.15 (br d, J = 12.4 Hz, 2H), 3.01-2.78 (m, 4H), 2.73- |
| | | | 2.55 (m, 3H), 2.48-2.29 (m, 2H), 2.28-2.15 (m, 1H), 2.13-1.94 |
| | | | (m, 4H), 1.93-1.77 (m, 4H), 1.76-1.62 (m, 1H), 1.61-1.44 (m, |
| | | | 3H), 1.06-0.97 (m, 2H), 0.78-0.68 (m, 2H) |
| 199 | 857 | 858 | δ 10.97 (s, 1H), 10.19 (s, 1H), 8.76-8.53 (m, 4H), 8.14 (s, 0.3H), |
| | | | 7.70-7.52 (m, 3H), 7.46-7.38 (m, 1H), 7.25 (br d, J = 1.5 Hz, 2H), |
| | | | 7.13 (s, 1H), 5.13-5.04 (m, 1H), 4.79 (br s, 1H), 4.44-4.35 (m, |
| | | | 1H), 4.31-4.23 (m, 1H), 3.82 (br s, 4H), 2.99-2.81 (m, 3H), 2.71- |
| | | | 2.56 (m, 2H), 2.46-2.31 (m, 3H), 2.15-1.94 (m, 8H), 1.93-1.81 |
| | | | (m, 2H), 1.79-1.66 (m, 2H), 1.65-1.49 (m, 3H), 1.04-0.94 (m, |
| | | | 2H), 0.78-0.70 (m, 2H) |
| 200 | 801 | 802 | δ 10.78 (s, 1H), 10.21 (s, 1H), 8.82-8.51 (m, 4H), 7.65 (br d, J = 7.4 |
| | | | Hz, 1H), 7.57 (s, 1H), 7.43 (t, J = 7.7 Hz, 1H), 7.24 (br d, J = 7.6 Hz, |
| | | | 1H), 6.96 (d, J = 8.4 Hz, 2H), 6.63 (d, J = 8.4 Hz, 2H), 5.70 (br d, J = |
| | | | 7.4 Hz, 1H), 4.51 (br d, J = 12.5 Hz, 1H), 4.27 (ddd, J = 4.9, 6.9, |
| | | | 11.3 Hz, 2H), 3.84 (br s, 3H), 3.21-3.04 (m, 3H), 2.83-2.59 (m, |
| | | | 5H), 2.29-1.94 (m, 7H), 1.93-1.69 (m, 5H), 1.67-1.30 (m, 3H), |
| | | | 1.01 (br dd, J = 1.9, 8.4 Hz, 2H), 0.74 (dd, J = 1.6, 4.9 Hz, 2H) |
| 201 | 831 | 832 | δ 10.10 (br d, J = 9.9 Hz, 2H), 8.66 (s, 1H), 8.59 (s, 2H), 8.36 (s, |
| | | | 1H), 7.63 (d, J = 8.3 Hz, 2H), 7.55 (s, 1H), 7.44-7.38 (m, 1H), 7.27- |
| | | | 7.20 (m, 2H), 7.14-7.09 (m, 1H), 5.08 (dd, J = 13.3, 5.4 Hz, 1H), |
| | | | 4.83-4.75 (m, 1H), 4.43-4.36 (m, 1H), 4.31-4.23 (m, 1H), 4.07- |
| | | | 4.00 (m, 1H), 3.95-3.86 (m, 2H), 3.26-3.20 (m, 2H), 3.15-3.08 |
| | | | (m, 1H), 2.90 (br d, J = 11.1 Hz, 2H), 2.42-2.32 (m, 3H), 2.09- |
| | | | 1.97 (m, 6H), 1.84-1.76 (m, 2H), 1.74-1.60 (m, 4H), 1.57-1.48 |
| | | | (m, 1H), 1.18 (t, J = 7.1 Hz, 1H), 1.03-0.97 (m, 2H), 0.76-0.71 (m, 2H) |
| 202 | 775 | 776 | δ 10.81-10.73 (m, 1H), 10.09 (br d, J = 6.6 Hz, 2H), 8.70-8.53 (m, |
| | | | 3H), 8.42-8.33 (m, 1H), 7.67-7.59 (m, 1H), 7.55 (s, 1H), 7.44- |
| | | | 7.36 (m, 1H), 7.22 (br d, J = 7.5 Hz, 1H), 7.01-6.90 (m, 2H), 6.62 |
| | | | (br d, J = 8.1 Hz, 2H), 5.67 (br d, J = 7.0 Hz, 1H), 4.55-4.45 (m, |
| | | | 1H), 4.32-4.14 (m, 2H), 3.10-3.00 (m, 2H), 2.97-2.87 (m, 2H), |
| | | | 2.65-2.58 (m, 3H), 2.40-2.31 (m, 2H), 2.10-1.98 (m, 4H), 1.88- |
| | | | 1.62 (m, 8H), 1.59-1.48 (m, 1H), 1.43-1.29 (m, 1H), 1.03-0.97 |
| | | | (m, 2H), 0.77-0.71 (m, 2H) |
| 203 | 815 | 816 | δ 11.00 (s, 1H), 10.19 (s, 1H), 10.14 (s, 1H), 8.68-8.64 (m, 1H), |
| | | | 8.62-8.60 (m, 2H), 8.38 (s, 1H), 8.16 (s, 1H), 7.65 (br d, J = 7.8 Hz, |
| | | | 2H), 7.56 (s, 1H), 7.50 (s, 1H), 7.43-7.39 (m, 2H), 7.23 (br d, J = |
| | | | 7.6 Hz, 1H), 5.11 (br dd, J = 13.2, 4.8 Hz, 1H), 4.46-4.38 (m, 2H), |
| | | | 4.31-4.16 (m, 2H), 3.14-2.87 (m, 6H), 2.70-2.66 (m, 2H), 2.42- |
| | | | 2.34 (m, 1H), 2.28-2.17 (m, 2H), 2.08-1.95 (m, 3H), 1.92-1.79 |
| | | | (m, 5H), 1.77-1.66 (m, 3H), 1.52-1.42 (m, 1H), 1.00 (dd, J = 8.2, |
| | | | 2.0 Hz, 2H), 0.76-0.72 (m, 2H) |
| 204 | 830 | 831 | δ 10.12 (d, J = 7.1 Hz, 2H), 8.67 (s, 1H), 8.62-8.56 (m, 2H), 8.37 |
| | | | (br s, 1H), 7.64 (br d, J = 7.7 Hz, 1H), 7.56 (s, 1H), 7.43-7.37 (m, |
| | | | 1H), 7.23-7.18 (m, 1H), 7.11-7.08 (m, 1H), 7.06-7.01 (m, 1H), |
| | | | 6.99-6.87 (m, 2H), 5.39-5.30 (m, 1H), 4.60-4.49 (m, 1H), 4.29- |
| | | | 4.17 (m, 1H), 3.33 (s, 3H), 3.14-3.06 (m, 2H), 2.98-2.90 (m, 3H), |
| | | | 2.88-2.81 (m, 1H), 2.74-2.58 (m, 4H), 2.36 (br dd, J = 11.5, 2.0 |
| | | | Hz, 1H), 2.12-1.96 (m, 5H), 1.87-1.79 (m, 4H), 1.74-1.66 (m, |
| | | | 3H), 1.00 (dd, J = 8.1, 2.0 Hz, 2H), 0.74 (br dd, J = 4.8, 1.5 Hz, 2H) |
| 205 | 857 | 858 | δ 10.99 (s, 1H), 10.21 (s, 1H), 8.74 (s, 1H), 8.71-8.66 (m, 2H), 8.57 |
| | | | (d, J = 2.1 Hz, 1H), 8.17 (s, 0.44H), 7.68-7.54 (m, 3H), 7.42 (t, J = |
| | | | 7.8 Hz, 1H), 7.30-7.17 (m, 2H), 7.07 (br d, J = 8.4 Hz, 1H), 5.09 |
| | | | (dd, J = 5.1, 13.3 Hz, 1H), 4.60-4.49 (m, 1H), 4.45-4.35 (m, 1H), |
| | | | 4.31-4.21 (m, 2H), 4.04 (br d, J = 13.4 Hz, 1H), 3.86 (br t, J = 6.9 |
| | | | Hz, 2H), 3.25 (br d, J = 13.1 Hz, 2H), 3.12 (br d, J = 13.2 Hz, 1H), |
| | | | 2.99-2.82 (m, 2H), 2.81-2.68 (m, 2H), 2.60 (br d, J = 16.7 Hz, |
| | | | 1H), 2.45-2.27 (m, 3H), 2.18 (br t, J = 6.7 Hz, 2H), 2.09-1.92 (m, |
| | | | 4H), 1.83 (br t, J = 10.5 Hz, 1H), 1.75-1.51 (m, 5H), 1.05-0.94 (m, |
| | | | 2H), 0.80-0.72 (m, 2H) |
| 206 | 801 | 802 | δ 10.81 (s, 1H), 10.40 (s, 1H), 9.64-9.33 (m, 1H), 8.87-8.60 (m, |
| | | | 4H), 7.64 (d, J = 7.8 Hz, 1H), 7.58 (s, 1H), 7.43 (t, J = 7.7 Hz, 1H), |
| | | | 7.25 (d, J = 7.7 Hz, 1H), 7.11-6.93 (m, 2H), 6.66 (br d, J = 8.4 Hz, |
| | | | 2H), 4.44 (br d, J = 13.2 Hz, 1H), 4.36-4.20 (m, 3H), 3.88 (br t, J = |
| | | | 6.4 Hz, 2H), 3.73-3.51 (m, 2H), 3.40-3.21 (m, 2H), 3.18-2.97 (m, |
| | | | 3H), 2.83-2.54 (m, 3H), 2.21 (br t, J = 6.4 Hz, 2H), 2.14-1.76 (m, |
| | | | 8H), 1.74-1.55 (m, 3H), 1.10-0.95 (m, 2H), 0.90-0.68 (m, 2H) |
| 207 | 841 | 842 | 10.98 (s, 1H), 10.19 (s, 1H), 8.74 (br s, 1H), 8.69-8.62 (m, 2H), |
| | | | 8.56 (d, J = 1.9 Hz, 1H), 7.63 (br d, J = 7.9 Hz, 2H), 7.56 (s, 1H), |
| | | | 7.47 (s, 1H), 7.44-7.35 (m, 2H), 7.21 (br d, J = 7.9 Hz, 1H), 5.09 |
| | | | (dd, J = 5.1, 13.2 Hz, 1H), 4.44-4.34 (m, 1H), 4.31-4.18 (m, 2H), |
| | | | 4.08 (br d, J = 13.6 Hz, 1H), 3.85 (br t, J = 6.9 Hz, 2H), 3.19-3.08 |
| | | | (m, 2H), 2.99-2.85 (m, 4H), 2.68-2.55 (m, 2H), 2.40-2.30 (m, |
| | | | 1H), 2.24-2.09 (m, 4H), 2.06-1.95 (m, 2H), 1.87-1.50 (m, 8H), |
| | | | 1.21 (br t, J = 7.1 Hz, 1H), 1.04-0.92 (m, 2H), 0.79-0.65 (m, 2H) |
| 208 | 856 | 857 | δ 11.11 (s, 1H), 10.23 (s, 1H), 8.81-8.46 (m, 4H), 7.70-7.54 (m, |
| | | | 2H), 7.43 (t, J = 7.7 Hz, 1H), 7.25 (br d, J = 7.9 Hz, 1H), 7.14-7.01 |
| | | | (m, 2H), 6.93 (br d, J = 7.9 Hz, 1H), 5.36 (br dd, J = 5.2, 12.4 Hz, |
| | | | 1H), 4.73-4.12 (m, 3H), 3.85 (br s, 3H), 3.66-3.52 (m, 2H), 3.24- |
| | | | 3.08 (m, 3H), 2.99-2.82 (m, 3H), 2.80-2.62 (m, 4H), 2.31-1.77 |
| | | | (m, 13H), 1.19-0.91 (m, 2H), 0.82-0.64 (m, 2H) |
| 209 | 856 | 857 | δ 11.08 (s, 1H), 10.17 (s, 1H), 8.81-8.44 (m, 4H), 7.66-7.51 (m, |
| | | | 2H), 7.40 (t, J = 7.7 Hz, 1H), 7.21 (br d, J = 7.8 Hz, 1H), 7.07-6.96 |
| | | | (m, 2H), 6.94-6.85 (m, 1H), 5.31 (dd, J = 5.3, 12.8 Hz, 1H), 4.28- |
| | | | 4.01 (m, 2H), 3.85 (br t, J = 6.8 Hz, 2H), 3.29 (s, 3H), 3.24-3.14 |
| | | | (m, 3H), 3.00-2.85 (m, 4H), 2.74-2.54 (m, 3H), 2.17 (br t, J = 6.6 |
| | | | Hz, 3H), 2.07-1.92 (m, 3H), 1.86-1.51 (m, 8H), 1.03-0.92 (m, |
| | | | 2H), 0.74-0.66 (m, 2H) |
| 210 | 830 | 831 | δ 11.11 (s, 1H), 10.21-10.17 (m, 1H), 10.17-10.11 (m, 1H), 8.69- |
| | | | 8.65 (m, 1H), 8.64-8.57 (m, 2H), 8.38 (s, 1H), 8.16 (s, 1H), 7.64 (br |
| | | | d, J = 7.6 Hz, 1H), 7.56 (s, 1H), 7.45-7.38 (m, 1H), 7.24 (br d, J = |
| | | | 7.8 Hz, 1H), 7.15-7.07 (m, 1H), 7.03 (br d, J = 8.1 Hz, 1H), 6.97- |
| | | | 6.87 (m, 1H), 5.43-5.26 (m, 1H), 4.44 (br d, J = 11.7 Hz, 1H), 4.24- |
| | | | 4.02 (m, 1H), 3.36 (br s, 4H), 3.25 (s, 1H), 3.13-3.01 (m, 3H), |
| | | | 2.96-2.86 (m, 1H), 2.72-2.60 (m, 4H), 2.36-2.22 (m, 2H), 2.06- |
| | | | 1.97 (m, 2H), 1.89-1.66 (m, 8H), 1.53-1.42 (m, 1H), 1.03-0.98 |
| | | | (m, 2H), 0.76-0.72 (m, 2H) |
| 211 | 787 | 788 | δ 10.67 (s, 1H), 10.08 (s, 1H), 8.69-8.40 (m, 4H), 8.10 (s, 0.6H), |
| | | | 7.61-7.45 (m, 2H), 7.32 (t, J = 7.8 Hz, 1H), 7.14 (d, J = 7.8 Hz, |
| | | | 1H), 6.86 (d, J = 8.4 Hz, 2H), 6.51 (d, J = 8.5 Hz, 2H), 5.56 (d, J = |
| | | | 7.4 Hz, 1H), 4.17 (ddd, J = 4.9, 7.0, 11.4 Hz, 1H), 3.71 (br t, J = 6.8 |
| | | | Hz, 3H), 2.94 (br d, J = 10.9 Hz, 3H), 2.78 (br d, J = 11.3 Hz, 2H), |
| | | | 2.70-2.56 (m, 1H), 2.53-2.46 (m, 1H), 2.31-2.20 (m, 2H), 2.06- |
| | | | 1.92 (m, 8H), 1.82-1.38 (m, 10H), 0.95-0.87 (m, 2H), 0.69-0.59 |
| | | | (m, 2H) |
| 212 | 780 | 781 | δ 10.66 (s, 1H), 9.53 (s, 1H), 8.53-8.43 (m, 2H), 8.38 (d, J = 2.0 |
| | | | Hz, 1H), 7.99 (s, 1H), 6.64 (d, J = 8.9 Hz, 2H), 6.49 (d, J = 8.9 Hz, |
| | | | 2H), 5.26 (d, J = 7.5 Hz, 1H), 4.22-4.02 (m, 3H), 3.70 (br t, J = 6.8 |
| | | | Hz, 2H), 3.28 (br s, 1H), 2.91-2.74 (m, 2H), 2.70-2.56 (m, 3H), |
| | | | 2.51-2.43 (m, 1H), 2.06 (br d, J = 7.0 Hz, 2H), 2.03-1.85 (m, 6H), |
| | | | 1.82-1.72 (m, 2H), 1.72-1.59 (m, 6H), 1.55-1.32 (m, 5H), 1.29- |
| | | | 1.19 (m, 1H), 1.15-1.06 (m, 2H), 0.76-0.62 (m, 1H), 0.45 (dq, J = |
| | | | 8.5, 4.6 Hz, 1H), 0.33-0.22 (m, 2H), 0.01 (q, J = 4.9 Hz, 2H) |

### BIOLOGICAL EXAMPLES

### Example B-1: Enzyme Binding Assays (KINOMEscan)

PIP4K2C binding constants disclosed herein were determined using the proprietary Human PIP4K2C binding assay (Catalog number 87-0007-1094, Eurofins DiscoverX, San Diego, CA). This assay measures active site-directed competition binding to an immobilized ligand (see https://doi.org/10.1038/nbt1068, herein incorporated by reference in its entirety). Experiments were conducted using an 11-point dose response curve, in duplicate, with a top starting concentration of 5 micromolar; the Levenberg-Marquardt algorithm was used to fit a Hill equation to dose-response data to calculate IC50 values (see Table 5).

TABLE 5
Legend: A = <500 nM, B = 500 nM-1 μM and C = 1 μM-5 μM

| Cmpd No. | Pip4k2c Kd: IC50 (nM) |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 24 | A |
| 26 | A |
| 27 | A |
| 28 | A |
| 29, Enant B | B |
| 29, Enant A | A |
| 30 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | B |
| 46 | A |
| 47 | A |
| 48 | A |
| 49 | A |
| 50 | A |
| 90 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | B |
| 105 | B |
| 106 | A |
| 107 | A |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | B |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |

### Example B-2: HiBiT PIP4K2C Degradation Assay

Materials: MOLT-4, a human T lymphoblast cell line (see https://www.atcc.org/products/crl-1582, herein incorporated by reference in its entirety), was genetically engineered using CRISPR-Cas9 by Synthego Corporation (Redwood City, CA) to C-terminally tag endogenous PIP4K2C with HiBiT peptide. Following clonal selection, genotype was confirmed by Sanger sequencing. MOLT-4 were maintained using RPMI-1640 medium from Gibco (Grand Island, NY, catalog number 11875-085) supplemented with 10% fetal bovine serum (FBS) from VWR (Philadelphia, PA, catalog number 97068-085). For HiBiT assays the cells were resuspended in RPMI-1640 medium without phenol read from Gibco (Grand Island, NY, catalog number 11835-030) supplemented with 10% FBS. Nano-Glo® HiBiT Lytic Assay System was purchased from Promega (Madison, WI, Catalog number N3030). Cell culture flasks and 384-well microplates were purchased from Corning (Glendale, AZ).

To study PIP4K2C degradation, 384-well plates were plated with a 10-point dose response curve, 3-fold dilution, in duplicate with a top starting concentration of 10 micromolar. Subsequently 22,500 cells were added to each well and incubated at 37C in 5% CO₂ for 24 hours. At the end of the incubation time, cells were treated with the Nano-Glo® HiBiT Lytic Detection System following the manufacturer's instructions. Luminescence was measured with a PHERAstar FSX plate reader (BMG Labtech, Cary, NC). The Levenberg-Marquardt algorithm was used to fit a Hill equation to dose-response data to calculate DC50 values (see Table 6).

TABLE 6
Legend: A = <500 nM, B = 500 nM-1 μM and C = 1 μM-5 μM

| | HiBIT-degradation MOLT4 |
|---|---|
| Cmpd No. | PIP4k2C 24 hours DC50 (nM) |
| 51 | C |
| 52 | B |
| 53 | A |
| 54 | A |
| 55 | B |
| 56 | A |
| 58 | A |
| 59 | B |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | B |
| 79 | A |
| 80 | A |
| 89 | A |
| 81 | A |
| 82 | A |
| 83 | C |
| 84 | A |
| 85 | B |
| 86 | A |
| 87 | A |
| 88 | A |
| 91 | C |
| 166 | A |
| 167 | A |
| 168 | A |
| 169 | A |
| 170 | A |
| 171 | A |
| 172 | A |
| 173 | A |
| 174 | C |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | B |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |

What is claimed is:

1. A compound of Formula (I′) 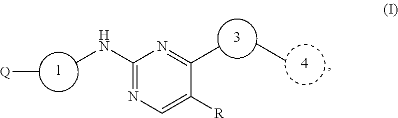
or a tautomer or stereoisomer thereof, or a pharmaceutically acceptable salt of any of the foregoing, wherein:
R is halo;
Ring 1 is pyridyl optionally substituted with one or more R¹;
Ring 3 is a ring selected from the group consisting of 3- to 10-membered cycloalkyl optionally substituted with one or more R^{3a}, 4- to 10-membered heterocyclyl optionally substituted with one or more R^{3a}, 6- to 10-membered aryl optionally substituted with one or more R^{3b}, or 5- to 10-membered heteroaryl optionally substituted with one or more R^{3b};
Ring 4 is optional, and when present is a ring selected from the group consisting of 3- to 10-membered cycloalkyl optionally substituted with one or more R^{4a}, 4- to 10-membered heterocyclyl optionally substituted with one or more R^{4a}, 6- to 10-membered aryl optionally substituted with one or more R^{4b}, and 5- to 10-membered heteroaryl optionally substituted with one or more R^{4b};
Q is Ring 5, wherein Ring 5 is a pyrrolidinone or imidazolidinone, each of which is optionally substituted with one or more R⁵, or
Q is of formula (i) 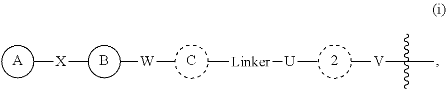
wherein:
Ring A is selected from the group consisting of 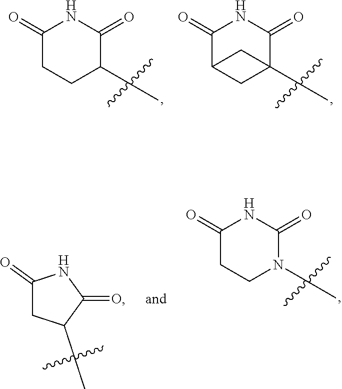 [SMILES: *C1CCC(=O)NC1=O ; *C12CC(C1)C(=O)NC2=O ; *C1CC(=O)NC1=O ; *N1CCC(=O)NC1=O]
each of which is optionally substituted with C₁₋₄alkyl or halo;
X is a bond, —O—, —NH—, or —NHC(O)—;
Ring B is selected from the group consisting of 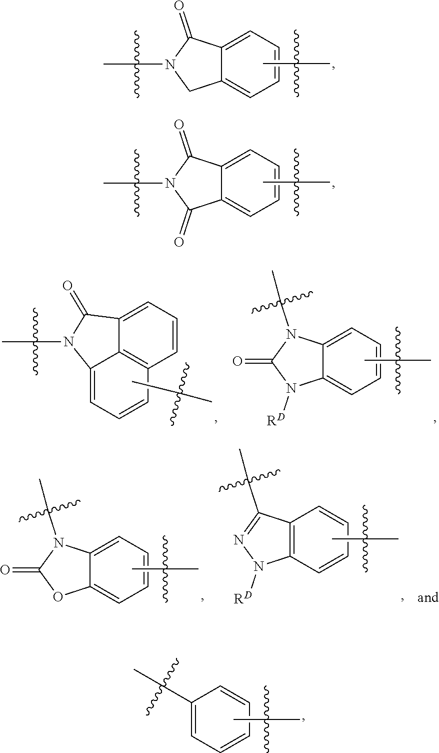
each of which is optionally substituted with one or more halo, C₁₋₄alkyl, C₁₋₄haloalkyl, or OH, wherein R^{D} is H or C₁₋₄alkyl;
W is a bond, —O—, —NH—, or —NHC(O)—;
Ring C is optional, and when present is 4- to 12-membered heterocyclyl optionally substituted with one or more R^{C};
Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
* indicates attachment to Ring C when Ring C is present, and * indicates attachment to W when Ring C is absent;
** indicates attachment to U when Ring 2 is present, and ** indicates attachment to V when Ring 2 is absent;
L¹ is a bond or C₁₋₆ alkylene;
L² is a 3- to 10-membered cycloalkyl or 4- to 12-membered heterocyclyl optionally substituted with one or more R^{L}; and
L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
U is absent when Ring 2 is absent, and is a bond or C(O) when Ring 2 is present;
Ring 2 is optional, and when present is selected from the group consisting of 3- to 10-membered cycloalkyl optionally substituted with one or more R^{2a}, 4- to 12-membered heterocyclyl optionally substituted with one or more R^{2a}, 6- to 12-membered aryl optionally substituted with one or more R^{2b}, and 5- to 12-membered heteroaryl optionally substituted with one or more R^{2b};
V is a bond, —NR^{V}—, —C(O)—, —C(O)NH—, or —NHC(O)—;
each R¹ is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)R^{w}, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-(3- to 6-membered cycloalkyl), C₁₋₄alkylene-N(R^{y})—C₁₋₄alkyl, C₁₋₄alkylene-S(O)ₙR^{x}, or oxo;
each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, O-phenyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R⁵ is independently C₁₋₄alkyl, or two R⁵ are taken together with the atom(s) to which they are attached to form a four- to six-membered ring which is optionally substituted with C₁₋₄alkyl or C(O)C₁₋₄alkyl;
each R^{C} is independently halo, OH, C₁₋₄alkyl, or C₁₋₄haloalkyl;
each R^{L} is independently halo, OH, C₁₋₄alkyl, or C₁₋₄haloalkyl;
each R^{V} is independently halo, OH, C₁₋₄alkyl, or C₁₋₄haloalkyl;
each R^{w} is independently 3- to 10-membered cycloalkyl, phenyl optionally substituted with halo, or 4- to 12-membered heterocyclyl optionally substituted with oxo;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
provided that, when Ring 3 and Ring 4 are each unsubstituted phenyl and Q is Ring 5, Ring 5 is substituted with one or more R⁵.

2. The compound of claim 1, wherein the compound of Formula (I′) is a compound of Formula (I) 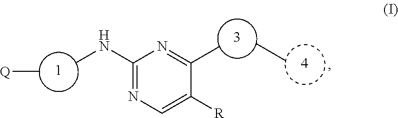
or a tautomer or stereoisomer thereof, or a pharmaceutically acceptable salt of any of the foregoing, wherein:
R is halo;
Ring 1 is pyridyl optionally substituted with one or more R¹;
Ring 3 is a monocyclic ring selected from the group consisting of 3- to 6-membered cycloalkyl optionally substituted with one or more R^{3a}, 4- to 6-membered heterocyclyl optionally substituted with one or more R^{3a}, phenyl optionally substituted with one or more R^{3b}, or 5- to 6-membered heteroaryl optionally substituted with one or more R^{3b};
Ring 4 is optional, and when present is a monocyclic ring selected from the group consisting of 3- to 6-membered cycloalkyl optionally substituted with one or more R^{4a}, 4- to 6-membered heterocyclyl optionally substituted with one or more R^{4a}, phenyl optionally substituted with one or more R^{4b}, and 5- to 6-membered heteroaryl optionally substituted with one or more R^{4b};
Q is Ring 5, wherein Ring 5 is a pyrrolidinone or imidazolidinone, each of which is optionally substituted with one or more R⁵, or
Q is of formula (i) 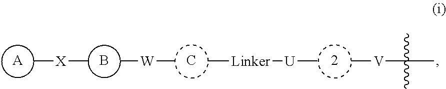
wherein:
Ring A is selected from the group consisting of 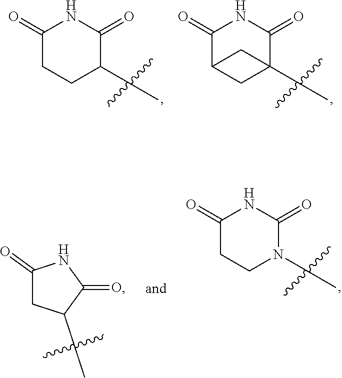 [SMILES: *C1CCC(=O)NC1=O ; *C12CC(C1)C(=O)NC2=O ; *C1CC(=O)NC1=O ; *N1CCC(=O)NC1=O]
each of which is optionally substituted with C₁₋₄alkyl or halo;
X is a bond, —O—, —NH—, or —NHC(O)—;
Ring B is selected from the group consisting of 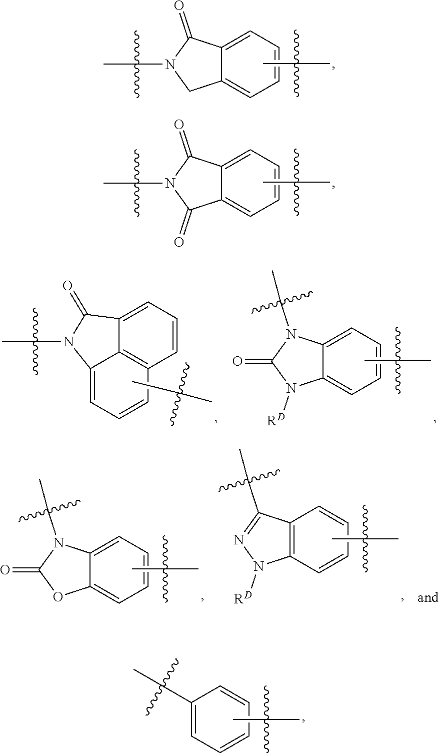
each of which is optionally substituted with one or more halo, C₁₋₄alkyl, C₁₋₄haloalkyl, or OH, wherein R^{D} is H or C₁₋₄alkyl;
W is a bond, —O—, —NH—, or —NHC(O)—;
Ring C is optional, and when present is 4- to 12-membered heterocyclyl optionally substituted with one or more R^{C};
Linker is a bond, C₁₋₁₀alkylene, C₁₋₁₀alkylene-N(R^{y}), C(O)C₁₋₁₀alkylene, C(O)C₁₋₁₀alkylene-N(R^{y}), C(O)N(R^{y})C₁₋₁₀alkylene, C(O)N(R^{y})C₁₋₁₀alkylene-N(R^{y}), C₁₋₆alkylene-C(O)C₁₋₁₀alkylene, C₁₋₆alkylene-C(O)C₁₋₁₀alkylene-N(R^{y}), C₁₋₆alkylene-C(O)N(R^{y})—C₁₋₁₀alkylene, C₁₋₆alkylene-C(O)N(R^{y})—C₁₋₁₀alkylene-N(R^{y}), or is of the formula *-L¹-L²-L³-**, wherein
* indicates attachment to Ring C when Ring C is present, and * indicates attachment to W when Ring C is absent;
** indicates attachment to U when Ring 2 is present, and ** indicates attachment to V when Ring 2 is absent;
L¹ is a bond or C₁₋₆ alkylene;
L² is a 3- to 10-membered cycloalkyl or 4- to 12-membered heterocyclyl optionally substituted with one or more R^{L}; and
L³ is C₁₋₁₀ alkylene, C₁₋₁₀ alkylene-N(R^{y}), C(O)—C₁₋₁₀alkylene, C(O)—C₁₋₁₀alkylene-N(R^{y}), C(O)N(R^{y})—C₁₋₁₀alkylene, or C(O)N(R^{y})—C₁₋₁₀alkylene-N(R^{y});
U is absent when Ring 2 is absent, and is a bond or C(O) when Ring 2 is present;
Ring 2 is optional, and when present is selected from the group consisting of 3- to 10-membered cycloalkyl optionally substituted with one or more R^{2a}, 4- to 12-membered heterocyclyl optionally substituted with one or more R², 6- to 12-membered aryl optionally substituted with one or more R^{2b}, and 5- to 12-membered heteroaryl optionally substituted with one or more R^{2b};
V is a bond, —NR^{V}—, —C(O)—, —C(O)NH—, or —NHC(O)—;
each R¹ is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R′), CN, N(R^{y})C(O)C₁₋₄alkyl, N(R^{y})C(O)C₁₋₄haloalkyl, SO₂R^{x}, SO₂N(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{2a}, R^{3a}, and R^{4a} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, C₁₋₄alkylene-S(O)ₙR^{x}, or oxo;
each R^{2b}, R^{3b}, and R^{4b} is independently halo, C₁₋₄alkyl, C₁₋₄haloalkyl, OH, C₁₋₄alkoxy, C₁₋₄haloalkoxy, C(O)C₁₋₄alkyl, C(O)N(R^{y})(R^{z}), CN, N(R^{y})C(O)C₁₋₄alkyl, N(R^{y})C(O)C₁₋₄haloalkyl, S(O)ₙR^{x}, S(O)ₙN(R^{y})(R^{z}), C₁₋₄alkylene-O—C₁₋₄alkyl, C₁₋₄alkylene-N(R^{y})—C₁₋₄alkyl, or C₁₋₄alkylene-S(O)ₙR^{x};
each R⁵ is independently C₁₋₄alkyl, or two R⁵ are taken together with the atom(s) to which they are attached to form a four- to six-membered ring which is optionally substituted with C₁₋₄alkyl or C(O)C₁₋₄alkyl;
each R^{C} is independently halo, OH, C₁₋₄alkyl, or C₁₋₄haloalkyl;
each R^{L} is independently halo, OH, C₁₋₄alkyl, or C₁₋₄haloalkyl;
each RV is independently halo, OH, C₁₋₄alkyl, or C₁₋₄haloalkyl;
each R^{x} is independently C₁₋₄alkyl, C₁₋₄haloalkyl, C₁₋₄alkylene-O—C₁₋₄alkyl, C₁₋₄alkylene-N(R^{y})—C₁₋₄alkyl, or C₁₋₄alkylene-S(O)ₙ—C₁₋₄alkyl;
each R^{y} and R^{z} is independently H, C₁₋₄alkyl, C₁₋₄haloalkyl, C₁₋₄alkylene-O—C₁₋₄alkyl, C₁₋₄alkylene-NH—C₁₋₄alkyl, C₁₋₄alkylene-N(C₁₋₄alkyl)-C₁₋₄alkyl, or C₁₋₄alkylene-S(O)ₙ—C₁₋₄alkyl; and
each n is independently 0, 1, or 2;
provided that, when Ring 3 and Ring 4 are each unsubstituted phenyl and Q is Ring 5, Ring 5 is substituted with one or more R⁵.

3. The compound of any of claim 1 or 2, wherein R is chloro.

4. The compound of any of claim 1 or 2, wherein R is fluoro.

5. The compound of any of claims 1-4, wherein Ring 1 is 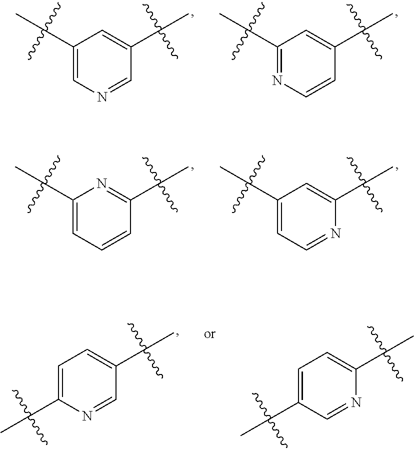 [SMILES: *c1cncc(*)c1 ; *c1ccnc(*)c1 ; *c1cccc(*)n1 ; *c1ccnc(*)c1 ; *c1ccc(*)nc1 ; *c1ccc(*)nc1]
each of which is optionally substituted with one or more R¹.

6. The compound of any of claims 1-5, wherein Ring 1 is

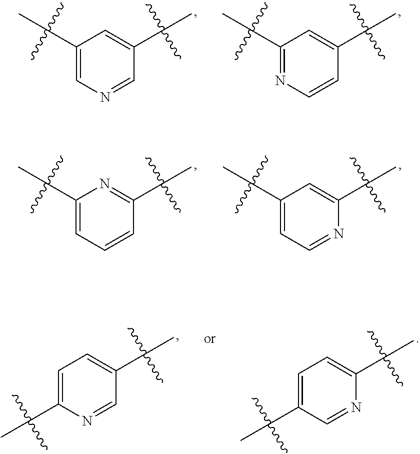 [SMILES: *c1cncc(*)c1 ; *c1ccnc(*)c1 ; *c1cccc(*)n1 ; *c1ccnc(*)c1 ; *c1ccc(*)nc1 ; *c1ccc(*)nc1]

7. The compound of any of claims 1-6, wherein Ring 1 is

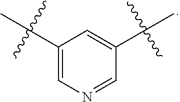 [SMILES: *c1cncc(*)c1]

8. The compound of any of claims 1-7, wherein Ring 3 is 4- to 10-membered heterocyclyl optionally substituted with one or more R^{3a}.

9. The compound of claim 8, wherein Ring 3 is 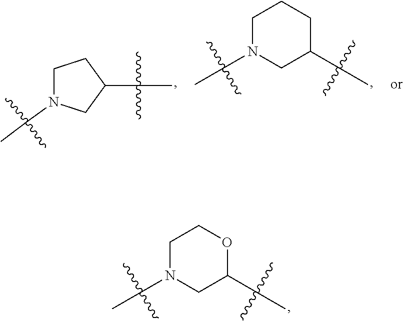 [SMILES: *C1CCN(*)C1 ; *C1CCCN(*)C1 ; *C1CN(*)CCO1]
each of which is optionally substituted with one or more R^{3a}.

10. The compound of any of claims 1-7, wherein Ring 3 is phenyl optionally substituted with one or more R^{3b}.

11. The compound of any of claims 1-7, wherein Ring 3 is 5- to 10-membered heteroaryl optionally substituted with one or more R^{3b}.

12. The compound of claim 11, wherein Ring 3 is 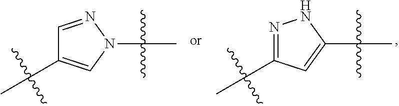 [SMILES: *c1cnn(*)c1 ; *c1cc(*)[nH]n1]
each of which is optionally substituted with one or more R^{3b}.

13. The compound of any of claims 1-12, wherein Ring 4 is absent.

14. The compound of any of claims 1-12, wherein Ring 4 is 3- to 10-membered cycloalkyl optionally substituted with one or more R^{4a}.

15. The compound of any of claims 1-12, wherein Ring 4 is 4- to 10-membered heterocyclyl optionally substituted with one or more R^{4a}.

16. The compound of claim 15, wherein Ring 4 is piperidinyl or pyrrolidinyl, each of which is optionally substituted with one or more R^{4a}.

17. The compound of any of claims 1-12, wherein Ring 4 is phenyl optionally substituted with one or more R^{4b}.

18. The compound of any of claims 1-12, wherein Ring 4 is 5- to 6-membered heteroaryl optionally substituted with one or more R^{4b}.

19. The compound of claim 18, wherein Ring 4 is pyridinyl or pyrazinyl, each of which is optionally substituted with one or more R^{4b}.

20. The compound of any of claims 1-19, wherein Q is Ring 5 and the compound is of Formula (II): 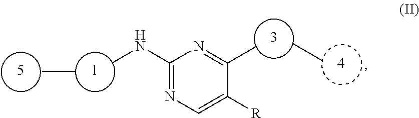
or a pharmaceutically acceptable salt thereof.

21. The compound of claim 20, wherein Ring 5 is 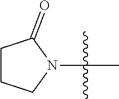 [SMILES: *N1CCCC1=O]
optionally substituted with one or more R⁵.

22. The compound of any of claim 20 or 21, wherein Ring 5 is

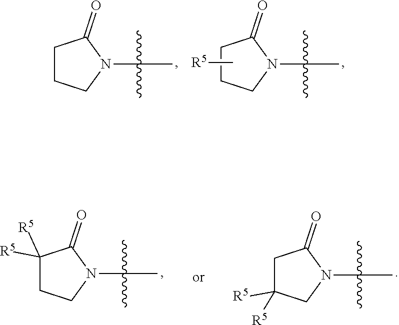

23. The compound of claim 20, wherein Ring 5 is 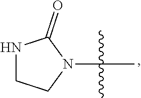 [SMILES: *N1CCNC1=O]
optionally substituted with one or more R⁵.

24. The compound of any of claims 20-23, wherein two R⁵ are taken together with the atom(s) to which they are attached to form a four- to six-membered ring which is optionally substituted with C₁₋₄alkyl or C(O)C₁₋₄alkyl.

25. The compound of any of claims 20-24, wherein two R⁵ are taken together with the atom(s) to which they are attached to form a four- to six-membered heterocyclyl ring which is optionally substituted with C₁₋₄alkyl or C(O)C₁₋₄alkyl.

26. The compound of any of claims 1-19, wherein Q is of Formula (i) and the compound is of Formula (III): 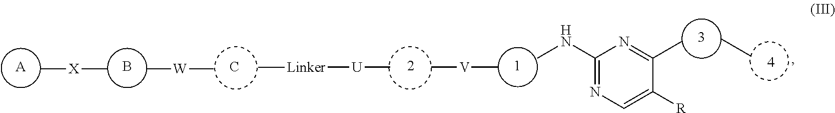
or a pharmaceutically acceptable salt thereof.

27. The compound of claim 26, wherein Ring A is 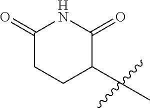 [SMILES: *C1CCC(=O)NC1=O]
optionally substituted with C₁₋₄alkyl or halo.

28. The compound of any of claim 26 or 27, wherein Ring B is 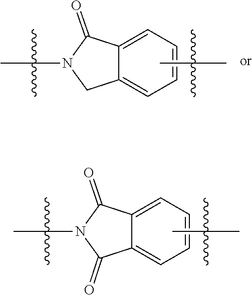
optionally substituted with one or more halo or OH.

29. The compound of any of claim 26 or 27, wherein Ring B is 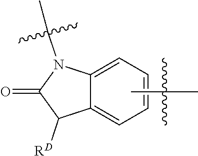
optionally substituted with one or more halo or OH, wherein R^{D} is H or C₁₋₄alkyl.

30. The compound of any of claim 26 or 27, wherein Ring B is 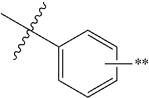
optionally substituted with one or more halo or OH.

31. The compound of any of claims 26-30, wherein Ring C is absent.

32. The compound of any of claims 26-30, wherein Ring C is piperidinyl optionally substituted with one or more R^{C}.

33. The compound of any of claims 26-30, wherein Ring C is piperazinyl optionally substituted with one or more R^{C}.

34. The compound of claim 26, wherein Ring A is 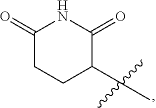 [SMILES: *C1CCC(=O)NC1=O]
X is a bond, Ring B is 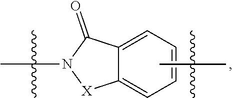
W is —O—, and Ring C is absent.

35. The compound of claim 26, wherein Ring A is 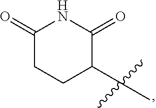 [SMILES: *C1CCC(=O)NC1=O]
X is a bond, Ring B is 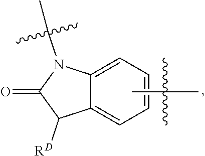
W is —O—, and Ring C is absent.

36. The compound of claim 26, wherein Ring A is 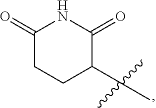 [SMILES: *C1CCC(=O)NC1=O]
X is —NH—, Ring B is 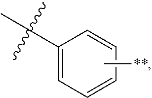
W is —O—, and Ring C is absent.

37. The compound of any of claims 26-36, wherein L is a bond, C₁₋₁₀alkylene, C(O)—C₁₋₁₀alkylene, C₁₋₆alkylene-C(O)—C₁₋₁₀alkylene, C₀₋₆alkylene-C(O)N(R^{y})—C₁₋₁₀alkylene, or L is L¹-L²-L³, wherein L¹ is a bond or C₁₋₆ alkylene, L² is a piperidinyl ring or piperazinyl ring, and L³ is C₁₋₁₀ alkylene.

38. The compound of any of claims 26-37, wherein Ring 2 is absent.

39. The compound of any of claims 26-37, wherein Ring 2 is 4- to 12-membered heterocyclyl optionally substituted with one or more R^{2a}.

40. The compound of claim 39, wherein Ring 2 is 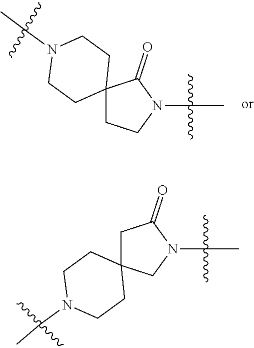 [SMILES: *N1CCC2(CC1)CCN(*)C2=O ; *N1CCC2(CC1)CC(=O)N(*)C2]
optionally substituted with one or more R^{2a}.

41. The compound of claim 1, wherein the compound is selected from the compounds provided in Table 1 and Table 2, a tautomer or stereoisomer thereof, or a pharmaceutically acceptable salt of any of the foregoing.

42. The compound of claim 41, wherein the compound is selected from the compounds provided in Table 1, a tautomer or stereoisomer thereof, or a pharmaceutically acceptable salt of any of the foregoing.

43. The compound of claim 41, wherein the compound is selected from the compounds provided in Table 2, a tautomer or stereoisomer thereof, or a pharmaceutically acceptable salt of any of the foregoing.

44. A pharmaceutical composition, comprising a compound of any of claims 1-43, a tautomer or stereoisomer thereof, or a pharmaceutically acceptable salt of any of the foregoing, and a pharmaceutically acceptable excipient.

45. A method of treating a disease or disorder associated with PIP4K2C, comprising administering to a subject a compound of any of claims 1-43, or the pharmaceutical composition of claim 44.

46. A method of treating a disease or disorder by modulating the level or activity of PIP4K2C, comprising administering to a subject a compound of any of claims 1-43, or a tautomer or stereoisomer thereof, or a pharmaceutically acceptable salt of any of the foregoing, or the pharmaceutical composition of claim 44.

47. A method of treating a disease or disorder by modulating the activity of PIP4K2C, comprising administering to a subject a compound of any of claims 20-25 or 42, or a tautomer or stereoisomer thereof, or a pharmaceutically acceptable salt of any of the foregoing, or the pharmaceutical composition of claim 44.

48. A method of treating a disease or disorder by modulating the level of PIP4K2C, comprising administering to a subject a compound of any of claims 26-40 or 43, or a tautomer or stereoisomer thereof, or a pharmaceutically acceptable salt of any of the foregoing, or the pharmaceutical composition of claim 44.

49. The method of any of claims 45-48, wherein the disease or disorder is a cancer, immune deficiency, autoimmune disease, infectious disease, or a combination thereof.

50. The method of claim 49, wherein the cancer is bladder cancer, breast cancer, triple negative breast cancer, cervical cancer, colorectal carcinoma, colorectal carcinoma with MSI, colorectal carcinoma with MSS, gastric cancer, head and neck squamous cell carcinoma, hepatocellular carcinoma, leukemia, lung cancer, small cell lung cancer, non-small cell lung cancer, lymphoma, melanoma, Merkel cell carcinoma, multiple myeloma, pancreatic ductal carcinoma, or renal cell carcinoma.

51. The method of claim 50, wherein the cancer is breast cancer, triple negative breast cancer, colorectal carcinoma, colorectal carcinoma with MSI, colorectal carcinoma with MSS, or non-small cell lung cancer.

52. The method of any of claims 45-48, wherein the disease or disorder is a neurodegenerative disease.

53. A method of treating a viral infection by modulating the level of PIP4K2C, comprising administering to a subject a compound of any of claims 26-40 or 43, or a tautomer or stereoisomer thereof, or a pharmaceutically acceptable salt of any of the foregoing, or the pharmaceutical composition of claim 44.

54. A method of preventing or treating necrotizing soft tissue infection (NSTI), comprising administering to a subject a compound of any of claims 26-40 or 43, or a tautomer or stereoisomer thereof, or a pharmaceutically acceptable salt of any of the foregoing, or the pharmaceutical composition of claim 44.

55. A method of modulating the level or activity of at least one of PIP4K2A, PIP4K2B, and PIP4K2C, comprising administering to a subject a compound of any of claims 1-43, or a tautomer or stereoisomer thereof, or a pharmaceutically acceptable salt of any of the foregoing, or the pharmaceutical composition of claim 44.

56. A method of modulating the activity of PIP4K2C, comprising administering to a subject a compound of any of claims 20-25 or 42, or a tautomer or stereoisomer thereof, or a pharmaceutically acceptable salt of any of the foregoing, or the pharmaceutical composition of claim 44.

57. A method of treating a disease or disorder by modulating the level of PIP4K2C, comprising administering to a subject a compound of any of claims 26-40 or 43, or a tautomer or stereoisomer thereof, or a pharmaceutically acceptable salt of any of the foregoing, or the pharmaceutical composition of claim 44.

58. A kit, comprising (i) a compound of any of claims 1-43, or a stereoisomer or tautomer thereof, or a pharmaceutically acceptable salt of any of the foregoing, or the pharmaceutical composition of claim 44, and (ii) instructions for use in treating an PIP4K-mediated disease, disorder, or condition in an individual in need thereof.
